SEQUENCE LISTING (21 sequences)

SEQ ID NO: 1 - protein, 25 aa, synthetic construct
GGGGSGGGGSGGGGSGGGGSGGGGS

SEQ ID NO: 2 - protein, 330 aa, synthetic construct [sequence omitted]

SEQ ID NO: 3 - protein, 107 aa, synthetic construct [sequence omitted]

SEQ ID NO: 4 - protein, 6 aa, synthetic construct
HHHHHH

SEQ ID NO: 5 - protein, 5 aa, synthetic construct
LPXTG

SEQ ID NO: 6 - protein, 116 aa, synthetic construct
EVQLVESGGGLVQPGGSLRLSCAASGFTFSDYGLHWVRQAPGKGLEWVSYISSGSSTVYFADTLKGRFTISRDNAKNSLYLQMNSLRAEDTAVYYCTRGTWYFDVWGRGTLVTVSS

SEQ ID NO: 7 - protein, 107 aa, synthetic construct
DIQMTQSPSSLSASVGDRVTITCRTSENIHSYLAWYQQKPGKSPQLLVYNAKTLADGVPSRFSGSGSGTDYTLTISSLQPEDFGTFYCQHFWTTSRTFGGGTKVEIK

SEQ ID NO: 8 - protein, 5 aa, synthetic construct
DYGLH

SEQ ID NO: 9 - protein, 17 aa, synthetic construct
YISSGSSTVYFADTLKG

SEQ ID NO: 10 - protein, 7 aa, synthetic construct
GTWYFDV

SEQ ID NO: 11 - protein, 11 aa, synthetic construct
RTSENIHSYLA

SEQ ID NO: 12 - protein, 7 aa, synthetic construct
NAKTLAD

SEQ ID NO: 13 - protein, 9 aa, synthetic construct
QHFWTTSRT

SEQ ID NO: 14 - protein, 120 aa, synthetic construct
QVQLVESGGGVVQPGRSLRLSCAASGFTFSSYGMHWVRQAPGKGLEWVAVIWYDGSNQYYADSVKGRFTISRDNSKNTLFLQMHSLRAEDTAVYYCARGLTSGRYGMDVWGQGTTVTVSS

SEQ ID NO: 15 - protein, 112 aa, synthetic construct
DIVMTQSPLSLPVTPGEPASISCRSSQSLLLSHGFNYLDWYLQKPGQSPQLLIYLGSSRASGVPDRFSGSGSGTDFTLKISRVEAEDVGLYYCMQPLQIPWTFGQGTKVEIK

SEQ ID NO: 16 - protein, 10 aa, synthetic construct
GFTFSSYGMH

SEQ ID NO: 17 - protein, 19 aa, synthetic construct
VAVIWYDGSNQYYADSVKG

SEQ ID NO: 18 - protein, 13 aa, synthetic construct
ARGLTSGRYGMDV

SEQ ID NO: 19 - protein, 16 aa, synthetic construct
RSSQSLLLSHGFNYLD

SEQ ID NO: 20 - protein, 6 aa, synthetic construct
LGSSRA

SEQ ID NO: 21 - protein, 9 aa, synthetic construct
MQPLQIPWT

(19) United States
(12) Patent Application Publication
(10) Pub. No.: US 2026/0034237 A1
(43) Pub. Date: Feb. 5, 2026

(54) LINKERS, DRUG LINKERS AND CONJUGATES THEREOF AND METHODS OF USING THE SAME

(71) Applicant: GENMAB A/S, Valby (DK)

(72) Inventors: Haidong LIU, Suzhou, Jiangsu (CN); Guobao WANG, Suzhou, Jiangsu (CN); Xiaolong JIANG, Suzhou, Jiangsu (CN); Xiao SHANG, Woodinville, WA (US)

(21) Appl. No.: 19/146,796

(22) PCT Filed: Jan. 11, 2024
(86) PCT No.: PCT/US2024/011307

§ 371 Date: Jul. 9, 2025

(30) Foreign Application Priority Data
Jan. 11, 2023 (WO) PCT/CN2023/071781

(51) Int. Cl. A61K 47/68; A61P 35/00

(52) U.S. Cl. CPC A61K 47/6889; A61K 47/68031; A61K 47/68037; A61P 35/00

(57) ABSTRACT

Embodiments of the present invention provide polar groups, linker compounds, linker drugs and conjugates thereof.

### CROSS REFERENCE TO RELATED APPLICATIONS

This application claims priority to International Application No. PCT/CN2023/071781 filed on Jan. 11, 2023, the entire contents of which are hereby incorporated by reference.

### BACKGROUND

A great deal of interest has surrounded the use of monoclonal antibodies (mAbs) for the targeted delivery of cytotoxic agents to cells associated with disease, such as cancer cells and other cells, in the form of antibody drug conjugates (or ADCs). The design of antibody drug conjugates, by attaching a cytotoxic agent, immune modulatory agent or other agent (collectively a “drug”) to an antibody, typically via a linker, involves consideration of a variety of factors. These factors include the identity and location of the chemical group for attachment of the drug, the mechanism of drug release, the structural element(s) (if any) providing release of the drug, and structural modification of the released free drug, if any. If the drug is released in the extracellular environment, the released form of the drug must able to reach its target. If the drug is to be released after antibody internalization, the structural elements and mechanism of drug release must be consonant with the intracellular trafficking of the conjugate.

Another important factor in the design of antibody drug conjugates is the amount of drug that can be delivered per targeting agent (i.e., the number of drugs attached to each targeting agent (e.g., an antibody), referred to as the drug load or drug loading). Historically, assumptions were that higher drugs loads were superior to lower drug loads (e.g., 8-loads vs 4-loads). The rationale was that higher loaded conjugates would deliver more drug (e.g., cytotoxic agent) to the target cells. This rationale was supported by the observations that conjugates with higher drug loadings were more active against cell lines in vitro. Certain later studies revealed, however, that this assumption was not confirmed in animal models. Conjugates having drug loads of 4 or 8 of certain auristatins were observed to have similar activities in mouse models. See, e.g., Hamblett et al., Clinical Cancer Res. 10:7063-70 (2004). Hamblett et al. further reported that the higher loaded ADCs were cleared more quickly from circulation in animal models. This faster clearance suggested a PK liability for higher loaded species as compared to lower loaded species. See Hamblett et al. In addition, higher loaded conjugates had lower maximum tolerated doses (MTDs) in mice, and as a result had narrower reported therapeutic indices. Id. In contrast, ADCs with a drug loading of 2 at engineered sites in a monoclonal antibody were reported to have the same or better PK and therapeutic indices as compared to certain 4-loaded ADCs. For example, see Junutula et al., Clinical Cancer Res. 16:4769 (2010). Thus, recent trends are to develop ADCs with low drug loadings.

There is a need, therefore, for antibody drug conjugate formats (and more generally for formats for other conjugates), that allow for higher drug loading, but that maintain other characteristics of lower loaded conjugates, such as favorable PK properties. Surprisingly, the present invention addresses those needs.

### SUMMARY OF THE INVENTION

Provided herein are Linkers having hydrophilic characteristics that maintain the intrinsic properties of antibodies conjugated with the Linkers and drugs. In particular, the Linkers aid in maintaining the hydrophilic properties of the antibodies when conjugated at higher drug loading and/or to hydrophobic drugs and other agents. Also provided are Drug-Linkers and conjugates comprising the Linkers, as well as methods of using such conjugates for the treatment of cancer and other diseases.

In some embodiments, provided is a Linker compound, comprising:
(a) a Linker unit having from 1 to 4 attachment sites for a Drug unit;
(b) an Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit, wherein the Polymer unit comprises the formula:

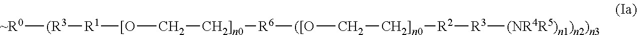

or a stereoisomer or salt thereof, wherein:
   R⁰ is a functional group for attachment to a subunit of the Amino Acid unit;
   each R¹ and R² are independently a bond or C₁-C₆ alkylene;
   each R³ is independently selected from a bond, C₁-C₁₂ alkylene, —C(O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —C₁-C₁₂ alkylene-NR^{a}—C(O)—, —C₁-C₁₂ alkylene-C(O)—NR^{a}—C₁-C₁₂ alkylene-, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, —NR^{a}—C(O)—C₁-C₁₂ alkylene-C(O)—, —C(O)—NR^{a}—C₁-C₁₂ alkylene-(CH(OH))₁₋₈—C₁-C₁₂ alkylene-, —O—CH₂—CH₂, —O—C(O)—NR^{a}—C₁-C₁₂ alkylene, —O—CH₂—CH(OH)—C(O)—, —O—CH₂—CH(OH)—C(O)—NR^{a}—C₁-C₁₂ alkylene-, —CH(OH)—, —CH(OH)—C₁-C₁₂ alkylene-, C₁-C₁₂ alkylene-CH(OH)—, —CH(OH)—C(O)—, —CH(OH)—C(O)—NR^{a}—C₁-C₁₂ alkylene-, —CH(OH)—C₁-C₁₂ alkylene-NR^{a}—C(O)—C₁-C₁₂ alkylene-C(O)—NR^{a}—C₁-C₁₂ alkylene-, —NR^{a}—C(O)—C₁-C₁₂ alkylene-C(O)—NR^{a}—C₁-C₁₂ alkylene-, —CH(OH)—NR^{a}—C₁-C₁₂ alkylene-, —[C(O)—(CH₂)₁₋₈—NR^{a}]₁₋₈—, triazolyl, —C₁-C₁₂ alkylene-triazolyl-, —N(polyhydroxyl group)-, and —C(O)NR⁷R⁸, wherein one of R⁷ and R⁸ is H or C₁-C₁₂ alkylene and the other is C₁-C₁₂ alkylene, each R^{a} is independently selected from H, C₁₋₆ alkyl, and wherein any of the above alkylene groups may be substituted with —SO₃H;
   each R⁴ and R⁵ are independently H, a polyhydroxyl group, a carboxyl-containing moiety, a substituted polyhydroxyl group, a —C(O)-polyhydroxyl group, a substituted —C(O)— polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
   each R⁶ is selected from:

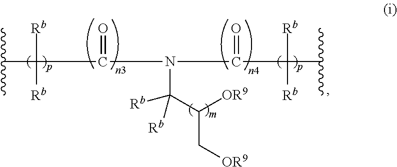

wherein:
   each n³ and n⁴ are independently 0-1,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6,
   m is 1-4,
   each v is independently 1-6, and
   n² is 1;

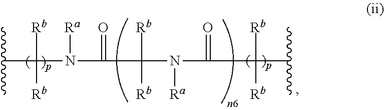

wherein:
   each R^{a} is independently H or C₁₋₆ alkyl,
   each R^{b} is independently H or C₁₋₆ alkyl,
   n⁶ is 1-10,
   each p is independently 0-6, and
   n² is 1;

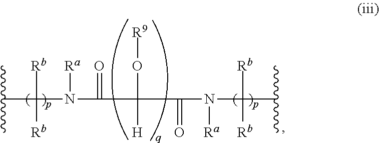

wherein:
   each R^{a} is independently H or C₁₋₆ alkyl,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6,
   q is 1-8,
   each v is independently 1-6, and
   n² is 1;

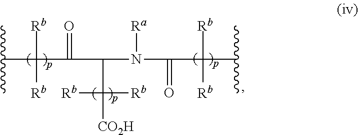

wherein:
   each R^{a} is independently H or C₁₋₆ alkyl,
   each R^{b} is independently H or C₁₋₆ alkyl,
   each p is independently 0-6, and
   n² is 1;
(v) —R¹⁰—[O—CH₂—CH₂]₁₋₈—R¹⁰—, wherein:
   each R^{b} is independently H or C₁₋₆ alkyl,
   each R¹⁰ is independently

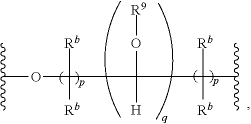

each p is independently 1-6,
each R⁹ is independently H, acetyl, —P(═O)(OH)₂, or —(CH₂)ᵥ—O—S(═O)₂(OH), and
q is 1-8;
n² is 1; and
(vi) —N—(R¹—X—R²—)₂, wherein:
   each X is independently —NR^{a}—C(O)— or —C(O)NR^{a}—, and
   n² is 2; and
the wavy line (˜) indicates the attachment site of the Amino Acid unit to R⁰;
each n⁰ is independently 2-26;
each n¹ is independently 1-6; and
n³ is 1-6.

In some embodiments, provided is a Linker compound, comprising:
(a) a Linker unit having from 1 to 4 attachment sites for a Drug unit;
(b) an Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit, wherein said Polymer unit comprises the formula:

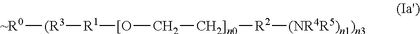

or a stereoisomer or salt thereof, wherein:
   R⁰ is a functional group for attachment to a subunit of the Amino Acid unit;
   each R¹ and R² are independently a bond or C₁-C₆ alkylene;
   each R³ is independently —N(polyhydroxyl group)-, triazolyl, —C₁-C₁₂ alkylene-triazolyl-,

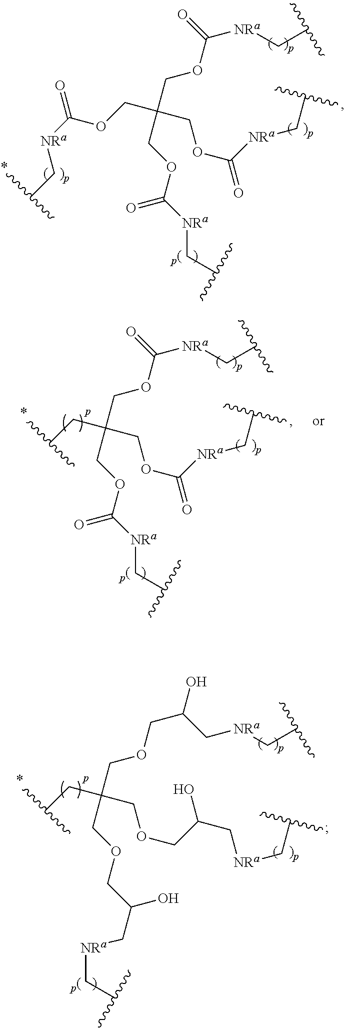

each R⁴ and R⁵ are independently H, a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
each R^{a} is independently H or C₁₋₆ alkyl;

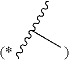

indicates the attachment site of R³ to R⁰
the wavy line

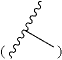

indicates the attachment site of the R³ to R¹;
each p is 1-6;
each n⁰ is independently 2-8;
each n¹ is independently 1-6; and
n³ is 1-6.

In some embodiments, provided is a Linker compound, comprising:
(a) a Linker unit having from 1 to 4 attachment sites for a Drug unit;
(b) an Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit, wherein said Polymer unit comprises the formula:

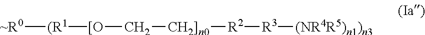

or a stereoisomer or salt thereof, wherein:
   (i) R⁰ is a functional group for attachment to a subunit of the Amino Acid unit;
      each R¹ and R² are independently a bond or C₁-C₆ alkylene;
      R³ is —C(O)—;
      R⁴ is H;
      R⁵ is independently a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate;
      the wavy line (˜) indicates the attachment site of the Amino Acid unit to R⁰;
      n⁰ is independently 2-26;
      n¹ is 1-6; and
      n³ is 1-6;
   (ii) R⁰ is —C(O)—;
      R¹, R², and R³ are each a bond;
      R⁴ and R⁵ are each independently H, a polyhydroxyl group, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
      the wavy line (˜) indicates the attachment site of the Amino Acid unit to R⁰;
      n⁰ is 6;
      n¹ is 1-6; and
      n³ is 1;
   (iii) R⁰ is a functional group for attachment to a subunit of the Amino Acid unit;
      R¹ and R² are each, independently, a bond or C₁-C₆ alkylene;
      R³ is —NRₐ—C(O)—C₁-C₁₂ alkylene-C(O)—, wherein the alkylene is substituted with —SO₃H;
      R^{a} is H or C₁₋₆ alkyl;
      R⁴ and R⁵ are each independently H, a carboxyl-containing moiety, a polyhydroxyl group, a substituted polyhydroxyl group, a —C(O)-polyhydroxyl group, a substituted —C(O)— polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
      the wavy line (˜) indicates the attachment site of the Amino Acid unit to R⁰;
      each n⁰ is independently 1-26;
      n¹ is 1-6; and
      n³ is 1-6; or
   (iv) R⁰ is

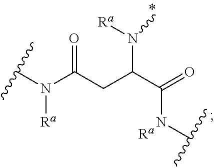

each R¹ is independently a bond or C₁-C₆ alkylene;
R² and R³ are each a bond;
R⁴ and R⁵ are each independently H, a polyhydroxyl group, a carboxyl-containing moiety, a substituted polyhydroxyl group, a —C(O)-polyhydroxyl group, a substituted —C(O)— polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
each R^{a} is independently H or C₁₋₆ alkyl;
the wavy line

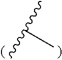

indicates the attachment site of R⁰ to the remainder of the Polymer unit;
the wavy line (˜*) indicates the attachment site of the Amino Acid unit to R⁰;
n⁰ is 1-8;
n¹ is 1-6; and
n³ is 2.

In some embodiments, provided is a Linker compound, comprising:
(a) a Linker unit having from 1 to 4 attachment sites for a Drug unit, said Linker unit comprising a moiety of formula:

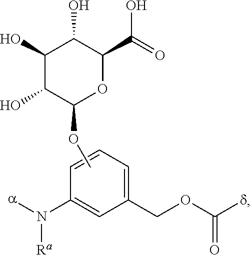

or a stereoisomer or salt thereof, wherein:
   α—represents a direct or indirect attachment site to an Amino Acid unit;
   δ—represents an attachment site to at least one of the Drug units or for a linking group attached to the at least one of the Drug units; and
R^{a} is H or C₁₋₆ alkyl;
(b) the Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit.

In some embodiments, provided is a Linker compound, comprising:
(a) a Linker unit having from 1 to 4 attachment sites for a Drug unit;
(b) an Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit, wherein said Polymer unit comprises:
   (i) a polyamide comprising the formula

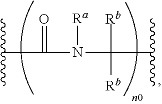

or a stereoisomer thereof, wherein each R^{a} is independently H or C₁₋₆ alkyl and each R^{b} is independently H or C₁₋₆ alkyl, and n⁰ is independently 2-26;
(ii) a polyether comprising the formula

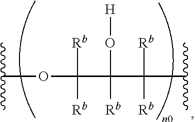

or a stereoisomer thereof, wherein each R^{b} is independently H or C₁₋₆ alkyl, and n⁰ is independently 2-26; or
(iii) combinations thereof.

In some embodiments, provided is a Linker compound, wherein at least one Polar group attached to the Amino Acid unit comprises the formula:

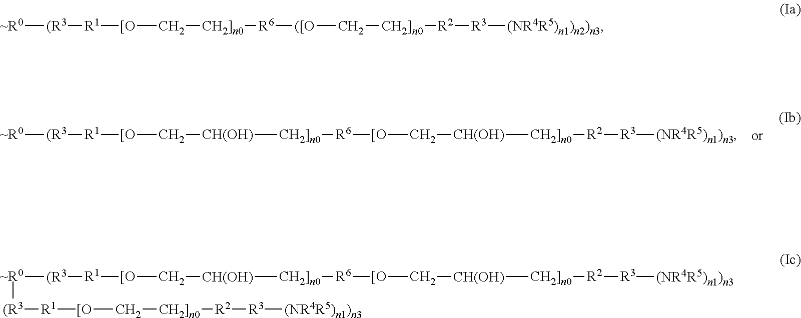

or a stereoisomer or salt thereof, wherein:
   R⁰ is a functional group for attachment to a subunit of the Amino Acid unit;
   each R¹ and R² are independently a bond or C₁-C₆ alkylene;
   each R³ is independently selected from a bond, C₁-C₁₂ alkylene, —C(O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —C₁-C₁₂ alkylene-NR^{a}—C(O)—, —C₁-C₁₂ alkylene-C(O)—NR^{a}—C₁-C₁₂ alkylene-, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, —NR^{a}—C(O)—C₁-C₁₂ alkylene-C(O)—, —C(O)—NR^{a}—C₁-C₁₂ alkylene-(CH(OH))₁₋₈—C₁-C₁₂ alkylene-, —O—CH₂—CH₂, —O—C(O)—NR^{a}—C₁-C₁₂ alkylene, —O—CH₂—CH(OH)—C(O)—, —O—CH₂—CH(OH)—C(O)—NR^{a}—C₁-C₁₂ alkylene-, —CH(OH)—, —CH(OH)—C₁-C₁₂ alkylene-, C₁-C₁₂ alkylene-CH(OH)—, —CH(OH)—C(O)—, —CH(OH)—C(O)—NR^{a}—C₁-C₁₂ alkylene-, —CH(OH)—C₁-C₁₂ alkylene-NR^{a}—C(O)—C₁-C₁₂ alkylene-C(O)—NR^{a}—C₁-C₁₂ alkylene-, —NR^{a}—C(O)—C₁-C₁₂ alkylene-C(O)—NR^{a}—C₁-C₁₂ alkylene-, —CH(OH)—NR^{a}—C₁-C₁₂ alkylene-, —[C(O)—(CH₂)₁₋₈—NR^{a}]₁₋₈—, triazolyl, —C₁-C₁₂ alkylene-triazolyl-, and —C(O)NR⁷R⁸, wherein one of R⁷ and R⁸ is H or C₁-C₁₂ alkylene and the other is C₁-C₁₂ alkylene, each R^{a} is independently selected from H, C₁₋₆ alkyl, and wherein any of the above alkylene groups may be substituted with —SO₃H;
   each R⁴ and R⁵ are independently H, a polyhydroxyl group, a carboxyl-containing moiety, a substituted polyhydroxyl group, a —C(O)-polyhydroxyl group, a substituted —C(O)— polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
   each R⁶ is independently a bond or selected from:

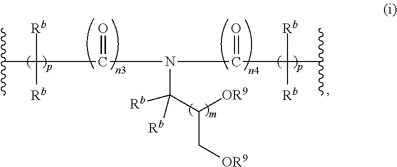

wherein:
   each n³ and n⁴ are independently 0-1,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6,
   m is 1-4, and
   each v is independently 1-6, and
   n² is 1;

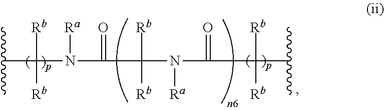

wherein:
   each R^{a} is independently H or C₁₋₆ alkyl,
   each R^{b} is independently H or C₁₋₆ alkyl,
   n⁶ is 1-10, and
   each p is independently 0-6, and
   n² is 1;

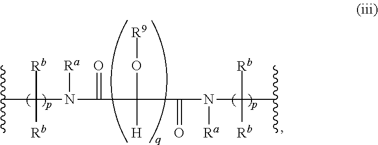

wherein:
   each R^{a} is independently H or C₁₋₆ alkyl,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6, and
   q is 1-8,
   each v is independently 1-6, and
   n² is 1;

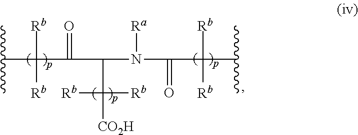

wherein:
   each R^{a} is independently H or C₁₋₆ alkyl,
   each R^{b} is independently H or C₁₋₆ alkyl, and
   each p is independently 0-6, and
   n² is 1;
   (v) —R¹⁰—[O—CH₂—CH₂]₁₋₈—R¹⁰—, wherein:
   each R^{b} is independently H or C₁₋₆ alkyl,
   each R¹⁰ is independently

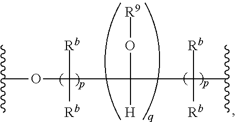

each p is independently 1-6, and
q is 1-8; and
(vi) —N—(R¹—X—R²—[O—CH₂—CH₂]ₙ₀—R²—R³—(NR⁴R⁵)ₙ₁)₂, wherein:
each X is independently —NR^{a}—C(O)— or —C(O)NR^{a}—, and
n² is 2; and
the wavy line (˜) indicates the attachment site of the Amino Acid unit to R⁰;
each n⁰ is independently 2-26;
n¹ is 0-6, and when n¹ is 0 then R³ is —OH or —C(O)OR, wherein R^{b} is independently H or C₁₋₆ alkyl; and
n³ is 1-6.

In some embodiments, provided are Drug-Linker compounds, comprising a Linker compound described herein with at least one Drug unit attached.

In some embodiments, provided are Conjugates comprising a Targeting unit attached to a Drug-Linker compound described herein.

In some embodiments, provided are pharmaceutical compositions comprising a Conjugate described herein and a pharmaceutically acceptable carrier.

In some embodiments, provided are methods of treating a subject in need thereof, comprising administering to the subject a Conjugate described herein or a pharmaceutical composition described herein, wherein the subject has cancer or an autoimmune disease and the Conjugate binds to a target molecule, such as a target antigen associated with the cancer or autoimmune disease.

These and other aspects of the present invention may be more fully understood by reference to the following detailed description, non-limiting examples of specific embodiments and the appended drawings.

### BRIEF DESCRIPTION OF THE DRAWINGS

The present disclosure is further described in terms of exemplary embodiments. These exemplary embodiments are described in detail with reference to the drawings. It should be noted that the drawings are not to scale. These embodiments are non-limiting exemplary embodiments, in which like reference numerals represent similar structures throughout the several views of the drawings, and wherein:

FIG. 1 is a graph illustrating in vitro cell cytotoxicity of mAb1-LD328 (8) and mAb2-vedotin (4) on cell line SW780;

FIG. 2 is a graph illustrating in vitro cell cytotoxicity of mAb1-LD328 (8) and mAb2-vedotin (4) on cell line CHP-212;

FIG. 3 is a graph illustrating in vivo efficacy of mAb1-LD328 (8) and mAb2-vedotin (4) in SW780 xenograft model; and

FIG. 4 is a graph illustrating in vivo efficacy of mAb1-LD328 (8) and mAb2-vedotin (4) in RT4 xenograft model.

### DEFINITIONS

For convenience, certain terms in the specification, examples and claims are defined here. Unless stated otherwise, or implicit from context, the following terms and phrases have the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context requires otherwise, throughout the description and the claims, the words “comprise”, “comprising”, and the like are to be construed in an inclusive sense as opposed to an exclusive or exhaustive sense; that is to say, in the sense of “including, but not limited to”.

The terms “decreased,” “reduce,” “reduced”, “reduction”, “decrease,” and “inhibit” are all used herein generally to mean a decrease by a statistically significant amount relative to a reference.

The terms “increased”, “increase” or “enhance” or “activate” are all used herein to generally mean an increase by a statically significant amount relative to a reference.

As used herein, the terms “protein” and “polypeptide” are used interchangeably herein to designate a series of amino acid residues each connected to each other by peptide bonds between the alpha-amino and carboxyl groups of adjacent residues. The terms “protein” and “polypeptide” also refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to an encoded gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, an “epitope” refers to the amino acids conventionally bound by an immunoglobulin VH/VL pair, such as the antibodies, antigen binding portions thereof and other binding agents described herein. Other binding agents comprise non-antibody scaffolds. An epitope can be formed on a polypeptide from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An epitope defines the minimum binding site for an antibody, antigen binding portions thereof and other binding agent, and thus represents the target of specificity of an antibody, antigen binding portion thereof or other immunoglobulin-based binding agent. In the case of a single domain antibody, an epitope represents the unit of structure bound by a variable domain in isolation.

As used herein, “specifically binds” refers to the ability of a binding agent (e.g., an antibody or antigen binding portion thereof) described herein to bind to a target with a KD of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. “Specifically binds” as stated herein also refers to the ability of a molecule (e.g., an antibody or antigen binding portion thereof or non-antibody scaffold) described herein to bind to a target with a KD of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the antibody, antigen binding portion or other binding agent and the concentration of target polypeptide. A person of ordinary skill in the art can determine appropriate conditions under which antibodies, antigen binding portions and other binding agents described herein selectively bind to a target molecule using any suitable methods, such as titration of an antibody or a binding agent in a suitable cell binding assay. A binding agent specifically bound to a target molecule is not displaced by a non-similar competitor. In certain embodiments, an antibody or antigen-binding portion thereof or other binding agent is said to specifically bind to a target molecule when it preferentially recognizes its target molecule in a complex mixture of proteins and/or macromolecules. Specific binding can be influenced by, for example, the affinity and avidity of the antibody, antigen binding portion or non-antibody scaffold and the concentration of target polypeptide. A person of ordinary skill in the art can determine appropriate conditions under which antibodies, antigen binding portions and non-antibody scaffolds described herein selectively bind to a target molecule using any suitable methods, such as titration of an antibody or a non-antibody scaffold in a suitable cell binding assay. A molecule specifically bound to a target molecule is not displaced by a non-similar competitor. In certain embodiments, an antibody or antigen-binding portion thereof or non-antibody scaffold is said to specifically bind to a target molecule when it preferentially recognizes its target molecule in a complex mixture of proteins and/or macromolecules.

Unless otherwise indicated, the term “alkyl” by itself or as part of another term refers to a substituted or unsubstituted straight chain or branched, saturated hydrocarbon having the indicated number of carbon atoms (e.g., “—C₁-C₅ alkyl”, “—C₁-C₈ alkyl” or “—C₁-C₁₀” alkyl refer to an alkyl group having from 1 to 5, 1 to 8, or 1 to 10 carbon atoms, respectively). Examples include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-I-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃.

Unless otherwise indicated, “alkenyl” by itself or as part of another term refers to a C₂-C₈ substituted or unsubstituted straight chain or branched, hydrocarbon with at least one site of unsaturation (i.e., a carbon-carbon, sp² double bond). Examples include, but are not limited to: ethylene or vinyl (—CH═CH₂), allyl (—CH₂CH═CH₂), cyclopentenyl (—C₅H₇), and 5-hexenyl (—CH₂CH₂CH₂CH₂CH═CH₂).

Unless otherwise indicated, “alkynyl” by itself or as part of another term refers to a refers to C₂-C₈, substituted or unsubstituted straight chain or branched, hydrocarbon with at least one site of unsaturation (i.e., a carbon-carbon, sp triple bond. Examples include, but are not limited to: acetylenic and propargyl.

Unless other indicated, “alkylene” refers to a saturated, branched or straight chain or hydrocarbon radical of 1-8 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like.

Unless otherwise indicated, “alkenylene” refers to an unsaturated, branched or straight chain hydrocarbon radical of 2-8 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

Unless otherwise indicated, “alkynylene” refers to an unsaturated, branched or straight chain or cyclic hydrocarbon radical of 2-8 carbon atoms, and having two monovalent radical centers derived by the removal of two hydrogen atoms from the same or two different carbon atoms of a parent alkyne. Typical alkynylene radicals include, but are not limited to: acetylene, propargyl, and 4-pentynyl.

Unless otherwise indicated, the term “heteroalkyl,” by itself or in combination with another term, refers to a substituted or unsubstituted stable straight or branched chain hydrocarbon, or combinations thereof, saturated and from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group (i.e., as part of the main chain) or at the position at which the alkyl group is attached to the remainder of the molecule. The heteroatom Si may be placed at any position of the heteroalkyl group, including the position at which the alkyl group is attached to the remainder of the molecule. Examples of heteroalkyl include the following: —CH₂CH₂OCH₃, —CH₂CH₂NHCH₃, —CH₂CH₂N(CH₃)CH₃, —CH₂SCH₂CH₃, CH₂CH₂S(O)CH₃, —CH₂CH₂S(O)₂CH₃, and —Si(CH₃)₃, —. Up to two heteroatoms may be consecutive, such as, for example, —CH₂NHOCH₃ and CH₂OSi(CH₃)₃. In some embodiments, a C₁ to C₄ heteroalkyl has 1 to 4 carbon atoms and 1 or 2 heteroatoms and a C₁ to C₃ heteroalkyl has 1 to 3 carbon atoms and 1 or 2 heteroatoms.

Unless otherwise indicated, the terms “heteroalkenyl” and “heteroalkynyl” by themselves or in combination with another term, refers to a substituted or unsubstituted stable straight or branched chain alkenyl or alkynyl having from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of a heteroalkenyl or heteroalkynyl group (i.e., as part of the main chain) or at the position at which the alkyl group is attached to the remainder of the molecule. The heteroatom Si may be placed at any position of a heteroalkenyl or heteroalkynyl group, including the position at which the alkyl group is attached to the remainder of the molecule.

Unless otherwise indicated, the term “heteroalkylene” by itself or as part of another substituent refers to a substituted or unsubstituted divalent group derived from a heteroalkyl (as discussed above), as exemplified by —CH₂CH₂SCH₂CH₂— and —CH₂SCH₂CH₂NHCH₂—. In some embodiments, a C₁ to C₄ heteroalkylene has 1 to 4 carbon atoms and 1 or 2 heteroatoms and a C₁ to C₃ heteroalkylene has 1 to 3 carbon atoms and 1 or 2 heteroatoms. For heteroalkylene groups, heteroatoms can also occupy either or both of the chain termini. Still further, for alkylene and heteroalkylene, no orientation is implied.

Unless otherwise indicated, the terms “heteroalkenylene” and “heteroalkynylene” by themselves or as part of another substituent refers to a substituted or unsubstituted divalent group derived from an heteroalkenyl or heteroalkynyl (as discussed above). In some embodiments, a C₂ to C₄ heteroalkenylene or heteroalkynylene has 1 to 4 carbon atoms. For heteroalkenylene and heteroalkynylene groups, heteroatoms can also occupy either or both of the chain termini. Still further, for alkylene and heteroalkenylene and heteroalkynylene, no orientation is implied.

Unless otherwise indicated, a “C₃-C₈ carbocycle,” by itself or as part of another term, refers to a substituted or unsubstituted 3-, 4-, 5-, 6-, 7- or 8-membered monovalent, substituted or unsubstituted, saturated or unsaturated non-aromatic monocyclic or bicyclic carbocyclic ring derived by the removal of one hydrogen atom from a ring atom of a parent ring system. Representative —C₃-C₈ carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl.

Unless otherwise indicated, a “C₃-C₈ carbocyclo”, by itself or as part of another term, refers to a substituted or unsubstituted C₃-C₈ carbocycle group defined above wherein another of the carbocycle groups' hydrogen atoms is replaced with a bond (i.e., it is divalent).

Unless otherwise indicated, a “C₃-C₁₀ carbocycle,” by itself or as part of another term, refers to a substituted or unsubstituted 3-, 4-, 5-, 6-, 7-, 8-, 9- or 10-membered monovalent, substituted or unsubstituted, saturated or unsaturated non-aromatic monocyclic, bicyclic or tricyclic carbocyclic ring derived by the removal of one hydrogen atom from a ring atom of a parent ring system. Representative —C₃-C₁₀ carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl. —C₃-C₁₀ carbocycles can further include fused cyclooctyne carbocycles, such as the fused cyclooctyne compounds disclosed in International Publication Number WO2011/136645 (the disclosure of which is incorporated by reference herein), including BCN (bicyclo[6.1.0]nonyne) and DBCO (Dibenzocyclooctyne).

Unless otherwise indicated, a “C₃-C₈ heterocycle,” by itself or as part of another term, refers to a substituted or unsubstituted monovalent substituted or unsubstituted aromatic or non-aromatic monocyclic or bicyclic ring system having from 3 to 8 carbon atoms (also referred to as ring members) and one to four heteroatom ring members independently selected from N, O, P or S, and derived by removal of one hydrogen atom from a ring atom of a parent ring system. One or more N, C or S atoms in the heterocycle can be oxidized. The ring that includes the heteroatom can be aromatic or nonaromatic. Unless otherwise noted, the heterocycle is attached to its pendant group at any heteroatom or carbon atom that results in a stable structure. Representative examples of a C₃-C₈ heterocycle include, but are not limited to, pyrrolidinyl, azetidinyl, piperidinyl, morpholinyl, tetrahydrofuranyl, tetrahydropyranyl, benzofuranyl, benzothiophene, indolyl, benzopyrazolyl, pyrrolyl, thiophenyl (thiophene), furanyl, thiazolyl, imidazolyl, pyrazolyl, pyrimidinyl, pyridinyl, pyrazinyl, pyridazinyl, isothiazolyl, and isoxazolyl. Unless otherwise indicate, the term “heterocarbocycle” is synonymous with the terms “heterocycle” or “heterocyclo” as described herein.

Unless otherwise indicated, “C₃-C₈ heterocyclo”, by itself or as part of another term, refers to a substituted or unsubstituted C₃-C₈ heterocycle group defined above wherein one of the heterocycle group's hydrogen atoms is replaced with a bond (i.e., it is divalent).

Unless otherwise indicated, “aryl” by itself or as part of another term, means a substituted or unsubstituted monovalent carbocyclic aromatic hydrocarbon radical of 6-20 carbon (preferably 6-14 carbon) atoms derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like. An exemplary aryl group is a phenyl group.

Unless otherwise indicated, an “arylene” by itself or as part of another term, is an unsubstituted or substituted aryl group as defined above wherein one of the aryl group's hydrogen atoms is replaced with a bond (i.e., it is divalent) and can be in the ortho, meta, or para orientations.

Unless otherwise indicated, “heteroaryl” and “heterocycle” refer to a ring system in which one or more ring atoms is a heteroatom, e.g., nitrogen, oxygen, and sulfur. A heterocycle radical comprises 1 to 20 carbon atoms and 1 to 3 heteroatoms selected from N, O, P, and S. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo[4,5], [5,5], [5,6], or [6,6] system.

Unless otherwise indicated, an “heteroarylene” by itself or as part of another term, is an unsubstituted or substituted heteroaryl group as defined above wherein one of the heteroaryl group's hydrogen atoms is replaced with a bond (i.e., it is divalent).

Unless otherwise indicated, “carboxyl” refers to COOH or COO⁻M⁺, where M⁺ is a cation.

Unless otherwise indicated, “oxo” refers to (C═O).

Unless otherwise indicated, “substituted alkyl” and “substituted aryl” mean alkyl and aryl, respectively, in which one or more hydrogen atoms are each independently replaced with a substituent. Typical substituents include, but are not limited to, —X, —R¹⁰, —O—, —OR¹⁰, —SR¹⁰, —S⁻, —NR¹⁰₂, —NR¹⁰₃, ═NR¹⁰, —CX₃, —CN, —OCN, —SCN, —N═C═O, —NCS, —NO, —NO₂, ═N₂, —N₃, —NR¹⁰C(═O)R¹⁰, —C(═O)R¹⁰, —C(═O)NR¹⁰₂, —SO₃—, —SO₃H, —S(═O)₂R¹⁰, —OS(═O)₂OR¹⁰, —S(═O)₂NR¹⁰, —S(═O)R¹⁰, —OP(═O)(OR¹⁰)₂, —P(═O)(OR¹⁰)₂, —PO⁻₃, —PO₃H₂, —AsO₂H₂, —C(═O)R¹⁰, —C(═O)X, —C(═S)R¹⁰, —CO₂R¹⁰, —CO₂—, —C(═S)OR¹⁰C(═O)SR¹⁰, C(═S)SR¹⁰, C(═O)NR¹⁰₂, C(═S)NR¹⁰₂, or C(═NR¹⁰)NR¹⁰₂, where each X is independently a halogen: —F, —Cl, —Br, or —I; and each R¹⁰ is independently —H, —C₁-C₂₀ alkyl, —C₆-C₂₀ aryl, —C₃-C₁₄ heterocycle, a protecting group or a prodrug moiety. Typical substitutents also include (═O). Alkylene, carbocycle, carbocyclo, arylene, heteroalkyl, heteroalkylene, heterocycle, and heterocyclo groups as described above may also be similarly substituted.

Unless otherwise indicated, “polyhydroxyl group” refers to an alkyl, alkylene, carbocycle or carbocyclo group including two or more, or three or more, substitutions of hydroxyl groups for hydrogen on carbon atoms of the carbon chain. In some embodiments, a polyhydroxyl group comprises at least three hydroxyl groups. In some embodiments, a polyhydroxyl group comprises carbon atoms containing only one hydroxyl group per carbon atom. A polyhydroxyl group may contain one or more carbon atoms that are not substituted with hydroxyl. A polyhydroxyl group may have each carbon atom substituted with a hydroxyl group. Examples of polyhydroxyl group includes linear (acyclic) or cyclic forms of monosaccharides such as C6 or C5 sugars, such as glucose, ribose, galactose, mannose, arabinose, 2-deoxyglucose, glyceraldehyde, erythrose, threose, xylose, lyxose, allose, altrose, gulose, idose, talose, aldose, and ketose, sugar acids such as gluconic acid, aldonic acid, uronic acid or ulosonic acid, and an amino sugars, such as glucosamine, N-acetyl glucosamine, galactosamine, and N-acetyl galactosamine. In some embodiments, polyhydroxyl group includes linear or cyclic forms of disaccharides and polysaccharides.

Unless otherwise indicated by context, “optionally substituted” refers to an alkyl, alkenyl, alkynyl, alkylaryl, arylalkyl heterocycle, aryl, heteroaryl, alkylheteroaryl, heteroarylalkyl, or other substituent, moiety or group as defined or disclosed herein wherein hydrogen atom(s) of that substituent, moiety or group has been optionally replaced with different moiety(ies) or group(s), or wherein an alicyclic carbon chain that comprise one of those substituents, moiety or group is interrupted by replacing carbon atom(s) of that chain with different moiety(ies) or group(s). In some aspects an alkene function group replaces two contiguous sp3 carbon atoms of an alkyl substituent, provided that the radical carbon of the alkyl moiety is not replaced, so that the optionally substituted alkyl is an unsaturated alkyl substituent.

Optional substituent replacing hydrogen(s) in any one of the foregoing substituents, moieties or groups is independently selected from the group consisting of aryl, heteroaryl, hydroxyl, alkoxy, aryloxy, cyano, halogen, nitro, fluoroalkoxy, and amino, including mono-, di- and tri-substituted amino groups, and the protected derivatives thereof, or is selected from the group consisting of —X, —OR′, —SR′, —NH₂, —N(R′)(R″), —N(R′)₃, ═NR, —CX₃, —CN, —NO₂, —NR′C(═O)H, —NR′C(═O)R, —NR′C(═O)R′, —C(═O)R′, —C(═O)NH₂, —C(═O)N(R′)R′, —S(═O)₂R′, —S(═O)₂NH₂, —S(═O)₂N(R′)R′, —S(═O)₂NH₂, —S(═O)₂N(R′)R″, —S(═O)₂OR′, —S(═O)R′, —OP(═O)(OR′)(OR′), —OP(OH)₃, —P(═O)(OR′)(OR′), —PO₃H₂, —C(═O)R′, —C(═S)R″, —CO₂R′, —C(═S)OR′, —C(═O)SR′, —C(═S)SR′, —C(═S)NH₂, —C(═S)N(R′)(R′)₂, —C(═NR′)NH₂, —C(═NR′)N(R′)R″, and salts thereof, wherein each X is independently selected from the group consisting of a halogen: —F, —Cl, —Br, and —I; and wherein each R is independently selected from the group consisting of C₁-C₂₀ alkyl, C₂-C₂₀ alkenyl, C₂-C₂₀ alkynyl, C₆-C₂₄ aryl, C₃-C₂₄ heterocyclyl (including C₅-C₂₄ heteroaryl), a protecting group, and a prodrug moiety or two of R″ together with the heteroatom to which they are attached defines a heterocyclyl; and R′ is hydrogen or R, wherein R″ is selected from the group consisting of C₁-C₂₀ alkyl, C₆-C₂₄ aryl, C₃-C₂₄ heterocyclyl (including C₅-C₂₄ heteroaryl), and a protecting group.

Typically, optional substituents are selected from the group consisting of —X, —OH, —OR″, —SH, —SR″, —NH₂, —NH(R″), —NR′(R″)₂, —N(R″)₃, ═NH, ═NR″, —CX₃, —CN, —NO₂, —NR′C(═O)H, NR′C(═O)R″—CO₂H, —C(═O)H, —C(═O)R″, —C(═O)NH₂, —C(═O)NR′R″— —S(═O)₂R″, —S(═O)₂NH₂, —S(═O)₂N(R′)R″, —S(═O)₂NH₂, —S(═O)₂N(R′)(R″), —S(═O)₂OR′, —S(═O)R″, —C(═S)R″, —C(═S)NH₂, —C(═S)N(R′)R″, —C(═NR′)N(R″)₂, and salts thereof, wherein each X is independently selected from the group consisting of —F and —Cl, R″ is typically selected from the group consisting of C₁-C₆ alkyl, C₆-C₁₀ aryl, C₃-C₁₀ heterocyclyl (including C₅-C₁₀ heteroaryl), and a protecting group; and R′ independently is hydrogen, C₁-C₆ alkyl, C₆-C₁₀ aryl, C₃-C₁₀ heterocyclyl (including C₅-C₁₀ heteroaryl), and a protecting group, independently selected from R″. More typically, substituents are selected from the group consisting of —X, —R″, —OH, —OR″, —NH₂, —NH(R″), —N(R″)₂, —N(R″)₃, —CX₃, —NO₂, —NHC(═O)H, —NHC(═O)R″, —C(═O)NH₂, —C(═O)NHR″, —C(═O)N(R″)₂, —CO₂H, —CO₂R, —C(═O)H, —C(═O)R″, —C(═O)NH₂, —C(═O)NH(R″), —C(═O)N(R″)₂, —C(═NR′)NH₂, —C(═NR′)NH(R″), —C(═NR′)N(R″)₂, a protecting group and salts thereof, wherein each X is —F, R″ is independently selected from the group consisting of C₁-C₆ alkyl, C₆-C₁₀ aryl, C₅-C₁₀ heteroaryl and a protecting group; and R′ is selected from the group consisting of hydrogen, C₁-C₆ alkyl and a protecting group, independently selected from R″.

The compounds of the invention, or their pharmaceutically acceptable salts may contain one or more asymmetric centers and may thus give rise to enantiomers, diastereomers, and other stereoisomeric forms that are defined, in terms of absolute stereochemistry, as (R) or (S) or, as (D) or (L) for amino acids. The present invention is meant to include all such possible isomers, as well as their racemic and optically pure forms. Optically active (+) and ( ), (R) and (S), or (D) and (L) isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposable mirror images of one another. The present invention also includes “diastereomers”, which refers to two or more stereoisomers of a compound that have different configurations at one or more of the equivalent stereocenters and are not mirror images of each other.

Although structures shown throughout the specification are depicted with specific stereocenters, the specification should be read to include variations in those stereocenters. For example, the structure of exatecan may be shown in the (S,S) configuration, but the (R,S) diastereomer of exatecan is also envisioned as being found in a separate embodiment of a conjugate as described herein.

The phrase “pharmaceutically acceptable salt,” as used herein, refers to pharmaceutically acceptable organic or inorganic salts of a compound (e.g., a Linker, Drug Linker, or a conjugate). The compound typically contains at least one amino group, and accordingly acid addition salts can be formed with this amino group. Exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, linleate, gentisinate, fumarate, gluconate, glucuronate, saccharate, formate, benzoate, glutamate, methanesulfonate, ethanesulfonate, benzenesulfonate, toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

As used herein, the term “consisting essentially of” refers to those elements required for a given embodiment. The term permits the presence of elements that do not materially affect the basic and novel or functional characteristic(s) of that embodiment.

As used herein, the term “consisting of” refers to compositions, methods, and respective components thereof as described herein, which are exclusive of any element not recited in that description of the embodiment.

Other than in the examples, or where otherwise indicated, all numbers expressing quantities of ingredients or reaction conditions used herein should be understood as modified in all instances by the term “about.” The term “about” when used in connection with percentages can mean +/−1%.

The terms “statistically significant” or “significantly” refer to statistical significance and generally mean a two standard deviation (2SD) difference, above or below a reference value.

Other terms are defined herein within the description of the various aspects of the invention.

### DETAILED DESCRIPTION

Provided herein are Linkers that comprise a Polar group, such as a Sugar unit, a Polymer unit, and/or a Carboxyl unit. Also provided are Targeting unit-Linkers, Drug Linkers, and conjugates thereof comprising Drug units, such as cytotoxic agents or immune modulatory agents, as further described herein.

In some embodiments, provided is a Linker compound, comprising:
(a) a Linker unit having from 1 to 4 attachment sites for a Drug unit;
(b) an Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit, wherein the Polymer unit comprises the formula:

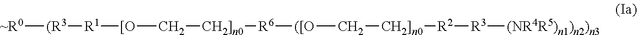

or a stereoisomer or salt thereof, wherein:
   R⁰ is a functional group for attachment to a subunit of the Amino Acid unit;
   each R¹ and R² are independently a bond or C₁-C₆ alkylene;
   each R³ is independently selected from a bond, C₁-C₁₂ alkylene, —C(O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —C₁-C₁₂ alkylene-NR^{a}—C(O)—, —C₁-C₁₂ alkylene-C(O)—NR^{a}—C₁-C₁₂ alkylene-, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, —NR^{a}—C(O)—C₁-C₁₂ alkylene-C(O)—, —C(O)—NR^{a}—C₁-C₁₂ alkylene-(CH(OH))₁₋₈—C₁-C₁₂ alkylene-, —O—CH₂—CH₂, —O—C(O)—NR^{a}—C₁-C₁₂ alkylene, —O—CH₂—CH(OH)—C(O)—, —O—CH₂—CH(OH)—C(O)—NR^{a}—C₁-C₁₂ alkylene-, —CH(OH)—, —CH(OH)—C₁-C₁₂ alkylene-, C₁-C₁₂ alkylene-CH(OH)—, —CH(OH)—C(O)—, —CH(OH)—C(O)—NR^{a}—C₁-C₁₂ alkylene-, —CH(OH)—C₁-C₁₂ alkylene-NR^{a}—C(O)—C₁-C₁₂ alkylene-C(O)—NR^{a}—C₁-C₁₂ alkylene-, —NR^{a}—C(O)—C₁-C₁₂ alkylene-C(O)—NR^{a}—C₁-C₁₂ alkylene-, —CH(OH)—NR^{a}—C₁-C₁₂ alkylene-, —[C(O)—(CH₂)₁₋₈—NR^{a}]₁₋₈—, triazolyl, —C₁-C₁₂ alkylene-triazolyl-, —N(polyhydroxyl group)-, and —C(O)NR⁷R⁸, wherein one of R⁷ and R⁸ is H or C₁-C₁₂ alkylene and the other is C₁-C₁₂ alkylene, each R^{a} is independently selected from H, C₁₋₆ alkyl, and wherein any of the above alkylene groups may be substituted with —SO₃H;
   each R⁴ and R⁵ are independently H, a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
   each R⁶ is selected from:

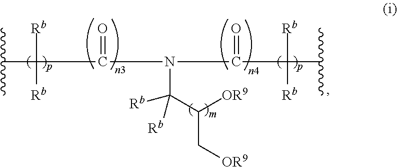

wherein:
   each n³ and n⁴ are independently 0-1,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6,
   m is 1-4,
   each v is independently 1-6, and
   n² is 1;

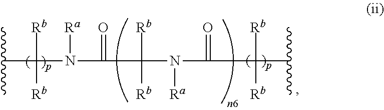

wherein:
   each R^{a} is independently H or C₁₋₆ alkyl,
   each R^{b} is independently H or C₁₋₆ alkyl,
   n⁶ is 1-10,
   each p is independently 0-6, and
   n² is 1;

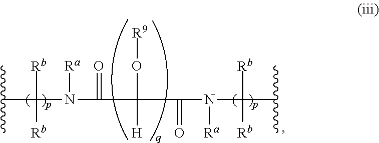

wherein:
   each R^{a} is independently H or C₁₋₆ alkyl,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6,
   q is 1-8,
   each v is independently 1-6, and
   n² is 1;

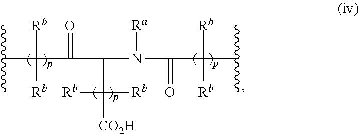

wherein:
   each R^{a} is independently H or C₁₋₆ alkyl,
   each R^{b} is independently H or C₁₋₆ alkyl,
   each p is independently 0-6, and
   n² is 1;
   (v) —R¹⁰—[O—CH₂—CH₂]₁₋₈—R¹⁰—, wherein:
   each R^{b} is independently H or C₁₋₆ alkyl,
   each R¹⁰ is independently

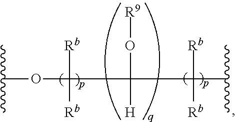

each p is independently 1-6,
each R⁹ is independently H, acetyl, —P(═O)(OH)₂, or —(CH₂)ᵥ—O—S(═O)₂(OH), and
q is 1-8;
n² is 1; and
(vi) —N—(R¹—X—R²—)₂, wherein:
each X is independently —NR^{a}—C(O)— or —C(O)NR^{a}—, and
n² is 2; and
the wavy line (˜) indicates the attachment site of the Amino Acid unit to R⁰;
each n⁰ is independently 2-26;
each n¹ is independently 1-6; and
n³ is 1-6.

In some embodiments, provided is a Linker compound, comprising:
(a) a Linker unit having from 1 to 4 attachment sites for a Drug unit;
(b) an Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit, wherein the Polymer unit comprises the formula:

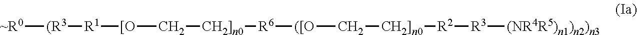

or a stereoisomer or salt thereof, wherein:
   R⁰ is a functional group for attachment to a subunit of the Amino Acid unit;
   each R¹ and R² are independently a bond or C₁-C₆ alkylene;
   each R³ is independently selected from a bond, C₁-C₁₂ alkylene, —C(O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —C₁-C₁₂ alkylene-NR^{a}—C(O)—, —C₁-C₁₂ alkylene-C(O)—NR^{a}—C₁-C₁₂ alkylene-, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, —NR^{a}—C(O)—C₁-C₁₂ alkylene-C(O)—, —C(O)—NR^{a}—C₁-C₁₂ alkylene-(CH(OH))₁₋₈—C₁-C₁₂ alkylene-, —O—CH₂—CH₂, —O—C(O)—NR^{a}—C₁-C₁₂ alkylene, —O—CH₂—CH(OH)—C(O)—, —O—CH₂—CH(OH)—C(O)—NR^{a}— C₁-C₁₂ alkylene-, —CH(OH)—, —CH(OH)—C₁-C₁₂ alkylene-, C₁-C₁₂ alkylene-CH(OH)—, —CH(OH)—C(O)—, —CH(OH)—C(O)—NR^{a}—C₁-C₁₂ alkylene-, —CH(OH)—C₁-C₁₂ alkylene-NR^{a}—C(O)—C₁-C₁₂ alkylene-C(O)—NR^{a}—C₁-C₁₂ alkylene-, —NR^{a}—C(O)—C₁-C₁₂ alkylene-C(O)—NR^{a}—C₁-C₁₂ alkylene-, —CH(OH)—NR^{a}—C₁-C₁₂ alkylene-, —[C(O)—(CH₂)₁₋₈—NR^{a}]₁₋₈—, triazolyl, —C₁-C₁₂ alkylene-triazolyl-, —N(polyhydroxyl group)-, and —C(O)NR⁷R⁸, wherein one of R⁷ and R⁸ is H or C₁-C₁₂ alkylene and the other is C₁-C₁₂ alkylene, each R^{a} is independently selected from H, C₁₋₆ alkyl, and
   wherein any of the above alkylene groups may be substituted with —SO₃H; each R⁴ and R⁵ are independently H, a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, optionally substituted C₃-C₁₀ carbocycle, optionally substituted C₁-C₃ alkylene C₃-C₁₀ carbocycle, optionally substituted heteroaryl, optionally substituted carbocycle, substituted —C₁-C₈ alkyl, substituted —C(O)—C₁-C₈ alkyl, —C(O)—R, where R is a Sugar unit of formula (XII) or (XIII), a chelator, or —NR⁴R⁵ join together to form a C₃-C₈ heterocycle, wherein optional substituents are selected from sulfate, phosphate, alkyl sulfate, and alkyl phosphate, and wherein at least one of R⁴ and R⁵ is not H;
   each R⁶ is selected from:

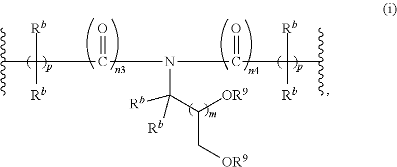

wherein:
   each n³ and n⁴ are independently 0-1,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6,
   m is 1-4,
   each v is independently 1-6, and
   n² is 1;

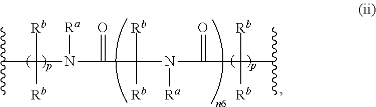

wherein:
   each R^{a} is independently H or C₁₋₆ alkyl,
   each R^{b} is independently H or C₁₋₆ alkyl,
   n⁶ is 1-10,
   each p is independently 0-6, and
   n² is 1;

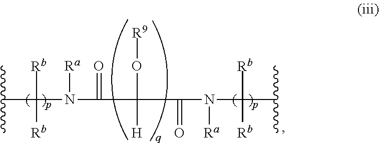

wherein:
   each R^{a} is independently H or C₁₋₆ alkyl,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6,
   q is 1-8,
   each v is independently 1-6, and
   n² is 1;

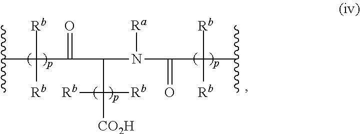

wherein:
   each R^{a} is independently H or C₁₋₆ alkyl,
   each R^{b} is independently H or C₁₋₆ alkyl,
   each p is independently 0-6, and
   n² is 1;
   (v) —R¹⁰—[O—CH₂—CH₂]₁₋₈—R¹⁰—, wherein:
   each R^{b} is independently H or C₁₋₆ alkyl,
   each R¹⁰ is independently

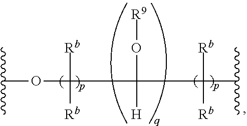

each p is independently 1-6,
each R⁹ is independently H, acetyl, —P(═O)(OH)₂, or —(CH₂)ᵥ—O—S(═O)₂(OH), and
q is 1-8;
n² is 1; and
(vi) —N—(R¹—X—R²—)₂, wherein:
each X is independently —NR^{a}—C(O)— or —C(O)NR^{a}—, and
n² is 2; and
the wavy line (˜) indicates the attachment site of the Amino Acid unit to R⁰;
each n⁰ is independently 2-26;
each n¹ is independently 1-6; and
n³ is 1-6.

In some embodiments, provided is a Linker compound, comprising:
(a) a Linker unit having from 1 to 4 attachment sites for a Drug unit;
(b) an Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit, wherein said Polymer unit comprises the formula:

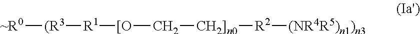

or a stereoisomer or salt thereof, wherein:
   R⁰ is a functional group for attachment to a subunit of the Amino Acid unit;
   each R¹ and R² are independently a bond or C₁-C₆ alkylene;
   each R³ is independently —N(polyhydroxyl group)-, triazolyl, —C₁-C₁₂ alkylene-triazolyl-,

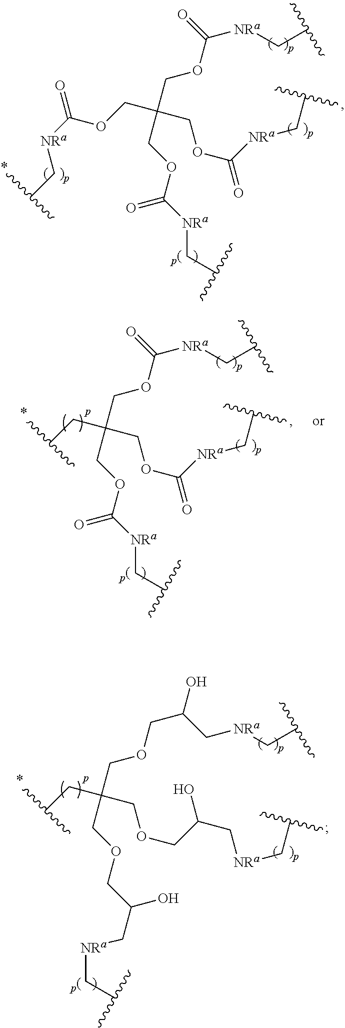

each R⁴ and R⁵ are independently H, a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
   each R^{a} is independently H or C₁₋₆ alkyl;

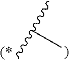

indicates the attachment site of R³ to R⁰
   the wavy line

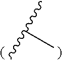

indicates the attachment site of the R³ to R¹;
   each p is 1-6;
   each n° is independently 2-8;
   each n¹ is independently 1-6; and
   n³ is 1-6.

In some embodiments, provided is a Linker compound, comprising:
(a) a Linker unit having from 1 to 4 attachment sites for a Drug unit;
(b) an Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit, wherein said Polymer unit comprises the formula:

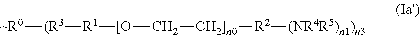

or a stereoisomer or salt thereof, wherein:
   R⁰ is a functional group for attachment to a subunit of the Amino Acid unit;
   each R¹ and R² are independently a bond or C₁-C₆ alkylene;
   each R³ is independently —N(polyhydroxyl group)-, triazolyl, —C₁-C₁₂ alkylene-triazolyl-,

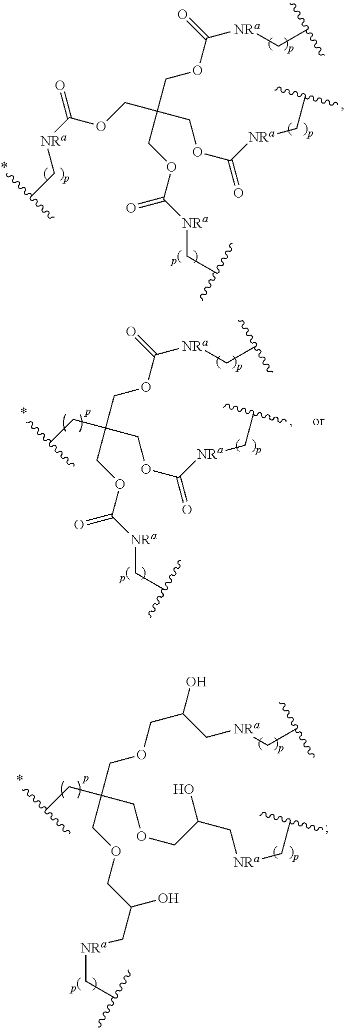

each R⁴ and R⁵ are independently H, a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, optionally substituted C₃-C₁₀ carbocycle, optionally substituted C₁-C₃ alkylene C₃-C₁₀ carbocycle, optionally substituted heteroaryl, optionally substituted carbocycle, substituted —C₁-C₈ alkyl, substituted —C(O)—C₁-C₈ alkyl, —C(O)—R, where R is a Sugar unit of formula (XII) or (XIII), a chelator, or —NR⁴R⁵ join together to form a C₃-C₈ heterocycle, wherein optional substituents are selected from sulfate, phosphate, alkyl sulfate, and alkyl phosphate, and wherein at least one of R⁴ and R⁵ is not H;
each R^{a} is independently H or C₁₋₆ alkyl;

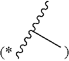

indicates the attachment site of R³ to R⁰
the wavy line

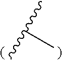

indicates the attachment site of the R³ to R¹;
each p is 1-6;
each n⁰ is independently 2-8;
each n¹ is independently 1-6; and
n³ is 1-6.

In some embodiments, provided is a Linker compound, comprising:
(a) a Linker unit having from 1 to 4 attachment sites for a Drug unit;
(b) an Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit, wherein said Polymer unit comprises the formula:

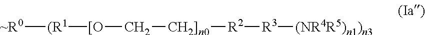

or a stereoisomer or salt thereof, wherein:
   (i) R⁰ is a functional group for attachment to a subunit of the Amino Acid unit; each R¹ and R² are independently a bond or C₁-C₆ alkylene;
      R³ is —C(O)—;
      R⁴ is H;
      R is independently a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate;
      the wavy line (˜) indicates the attachment site of the Amino Acid unit to R⁰;
      n⁰ is independently 2-26;
      n¹ is 1-6; and
      n³ is 1-6;
   (ii) R⁰ is —C(O)—;
      R¹, R², and R³ are each a bond;
      R⁴ and R⁵ are each independently H, a polyhydroxyl group, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
      the wavy line (˜) indicates the attachment site of the Amino Acid unit to R⁰;
      n⁰ is 6;
      n¹ is 1-6; and
      n³ is 1;
   (iii) R⁰ is a functional group for attachment to a subunit of the Amino Acid unit;
      R¹ and R² are each, independently, a bond or C₁-C₆ alkylene;
      R³ is-NR^{a}—C(O)—C₁-C₁₂ alkylene-C(O)—, wherein the alkylene is substituted with —SO₃H;
      R^{a} is H or C₁₋₆ alkyl;
      R⁴ and R⁵ are each independently H, a carboxyl-containing moiety, a polyhydroxyl group, a substituted polyhydroxyl group, a —C(O)-polyhydroxyl group, a substituted —C(O)— polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
      the wavy line (˜) indicates the attachment site of the Amino Acid unit to R⁰;
      each n⁰ is independently 1-26;
      n¹ is 1-6; and
      n³ is 1-6; or
   (iv) R⁰ is

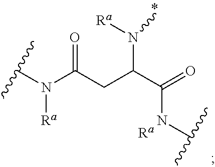

each R¹ is independently a bond or C₁-C₆ alkylene;
R² and R³ are each a bond;
R⁴ and R⁵ are each independently H, a polyhydroxyl group, a carboxyl-containing moiety, a substituted polyhydroxyl group, a —C(O)-polyhydroxyl group, a substituted —C(O)— polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
each R^{a} is independently H or C₁₋₆ alkyl;
the wavy line

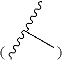

indicates the attachment site of R⁰ to the remainder of the Polymer unit;
the wavy line (˜*) indicates the attachment site of the Amino Acid unit to R⁰; n⁰ is 1-8;
n¹ is 1-6; and
n³ is 2.

In some embodiments, provided is a Linker compound, comprising:
(a) a Linker unit having from 1 to 4 attachment sites for a Drug unit;
(b) an Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit, wherein said Polymer unit comprises the formula:

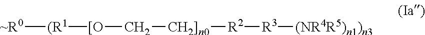

or a stereoisomer or salt thereof, wherein:
   (i) R⁰ is a functional group for attachment to a subunit of the Amino Acid unit;
      each R¹ and R² are independently a bond or C₁-C₆ alkylene;
      R³ is —C(O)—;
      R⁴ is H;
      R⁵ is independently H, a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, optionally substituted C₃-C₁₀ carbocycle, optionally substituted C₁-C₃ alkylene C₃-C₁₀ carbocycle, optionally substituted heteroaryl, optionally substituted carbocycle, substituted —C₁-C₈ alkyl, substituted —C(O)—C₁-C₈ alkyl, —C(O)—R, where R is a Sugar unit of formula (XII) or (XIII), a chelator, or —NR⁴R⁵ join together to form a C₃-C₈ heterocycle, wherein optional substituents are selected from sulfate, phosphate, alkyl sulfate, and alkyl phosphate;
      the wavy line (˜) indicates the attachment site of the Amino Acid unit to R⁰;
      n⁰ is independently 2-26;
      n¹ is 1-6; and
      n³ is 1-6;
   (ii) R⁰ is —C(O)—;
      R¹, R², and R³ are each a bond;
      R⁴ and R⁵ are each independently H, a polyhydroxyl group, a substituted polyhydroxyl group, a —C(O)-polyhydroxyl group, a substituted —C(O)-polyhydroxyl group, a polyhydroxyl-ether group, a substituted polyhydroxyl-ether group, optionally substituted C₃-C₁₀ carbocycle, optionally substituted C₁-C₃ alkylene C₃-C₁₀ carbocycle, optionally substituted heteroaryl, optionally substituted carbocycle, substituted —C₁-C₈ alkyl, substituted —C(O)—C₁-C₈ alkyl, —C(O)—R, where R is a Sugar unit of formula (XII) or (XIII), a chelator, or —NR⁴R⁵ join together to form a C₃-C₈ heterocycle, wherein optional substituents are selected from sulfate, phosphate, alkyl sulfate, and alkyl phosphate, and wherein at least one of R⁴ and R⁵ is not H;
      the wavy line (˜) indicates the attachment site of the Amino Acid unit to R⁰;
      n⁰ is 6;
      n¹ is 1-6; and
      n³ is 1;
   (iii) R⁰ is a functional group for attachment to a subunit of the Amino Acid unit;
      R¹ and R² are each, independently, a bond or C₁-C₆ alkylene;
      R³ is-NRₐ—C(O)—C₁-C₁₂ alkylene-C(O)—, wherein the alkylene is substituted with —SO₃H;
      R^{a} is H or C₁₋₆ alkyl;
      R⁴ and R⁵ are each independently H, a carboxyl-containing moiety, a polyhydroxyl group, a substituted polyhydroxyl group, a —C(O)-polyhydroxyl group, a substituted —C(O)— polyhydroxyl group, a polyhydroxyl-ether group, a substituted polyhydroxyl-ether group, optionally substituted C₃-C₁₀ carbocycle, optionally substituted C₁-C₃ alkylene C₃-C₁₀ carbocycle, optionally substituted heteroaryl, optionally substituted carbocycle, substituted —C₁-C₈ alkyl, substituted —C(O)—C₁-C₈ alkyl, —C(O)—R, where R is a Sugar unit of formula (XII) or (XIII), a chelator, or —NR⁴R⁵ join together to form a C₃-C₈ heterocycle, wherein optional substituents are selected from sulfate, phosphate, alkyl sulfate, and alkyl phosphate, and wherein at least one of R⁴ and R⁵ is not H;
      the wavy line (˜) indicates the attachment site of the Amino Acid unit to R⁰;
      each n⁰ is independently 1-26;
      n¹ is 1-6; and
      n³ is 1-6; or
   (iv) R⁰ is a

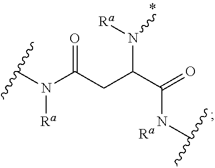

each R¹ is independently a bond or C₁-C₆ alkylene;
R² and R³ are each a bond;
R⁴ and R⁵ are each independently H, a polyhydroxyl group, a carboxyl-containing moiety, a substituted polyhydroxyl group, a —C(O)-polyhydroxyl group, a substituted —C(O)— polyhydroxyl group, a polyhydroxyl-ether group, a substituted polyhydroxyl-ether group, a chelator, or —NR⁴R⁵ join together to form a C₃-C₈ heterocycle, wherein optional substituents are selected from sulfate, phosphate, alkyl sulfate, and alkyl phosphate, and wherein at least one of R⁴ and R⁵ is not H;
each R^{a} is independently H or C₁₋₆ alkyl;
the wavy line

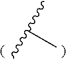

indicates the attachment site of R⁰ to the remainder of the Polymer unit;
the wavy line (˜*) indicates the attachment site of the Amino Acid unit to R⁰;
n⁰ is 1-8;
n¹ is 1-6; and
n³ is 2.

In some embodiments, provided is a Linker compound, comprising:
(a) a Linker unit having from 1 to 4 attachment sites for a Drug unit, said Linker unit comprising a moiety of formula:

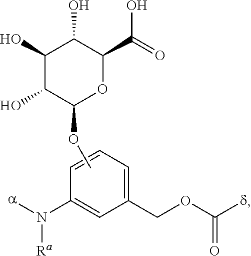

or a stereoisomer or salt thereof, wherein:
   α—represents a direct or indirect attachment site to an Amino Acid unit;
   δ—represents an attachment site for at least one of the Drug units or for a linking group attached to the at least one Drug units; and
R^{a} is H or C₁₋₆ alkyl;
(b) the Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit.

In some embodiments, provided is a Linker compound, comprising:
(a) a Linker unit having from 1 to 4 attachment sites for a Drug unit, said Linker unit comprising a moiety of formula:

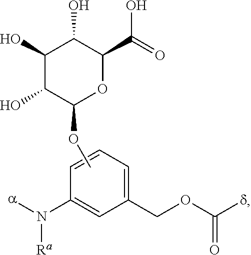

or a stereoisomer or salt thereof, wherein:
   α—represents a direct or indirect attachment site to an Amino Acid;
   δ—represents an attachment site to at least one of the Drug units or for a linking group attached to the at least one of the Drug units; and
R^{a} is H or C₁₋₆ alkyl;
(b) the Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit.

In some embodiments, the Linker unit comprises a moiety of formula:

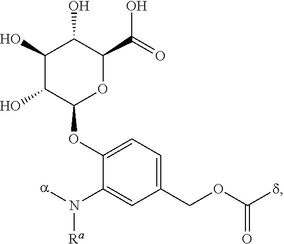

or a stereoisomer or salt thereof.

In some embodiments, the Linker unit comprises a moiety of formula:

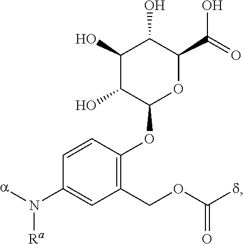

or a stereoisomer or salt thereof.

In some embodiments, the Linker unit comprises a moiety of formula:

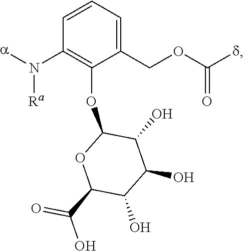

or a stereoisomer or salt thereof.

In some embodiments, provided is a Linker compound, comprising:
(a) a Linker unit having from 1 to 4 attachment sites for a Drug unit;
(b) an Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit, wherein said Polymer unit comprises:
   (i) an optionally substituted polyamide;
   (ii) a substituted polyether; or
   (iii) combinations thereof.

In some embodiments, provided is a Linker compound, comprising:
(a) a Linker unit having from 1 to 4 attachment sites for a Drug unit;
(b) an Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit, wherein said Polymer unit comprises:
   (i) a polyamide comprising the formula

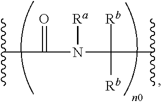

or a stereoisomer thereof, wherein each R^{a} is independently H or C₁₋₆ alkyl and each R^{b} is independently H or C₁₋₆ alkyl, and n⁰ is independently 2-26;
(ii) a polyether comprising the formula

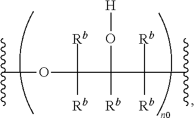

or a stereoisomer thereof, wherein each R^{b} is independently H or C₁₋₆ alkyl, and n⁰ is independently 2-26; or
(iii) combinations thereof.

In some embodiments, provided is a Linker compound, wherein at least one Polar group attached to the Amino Acid unit comprises the formula:

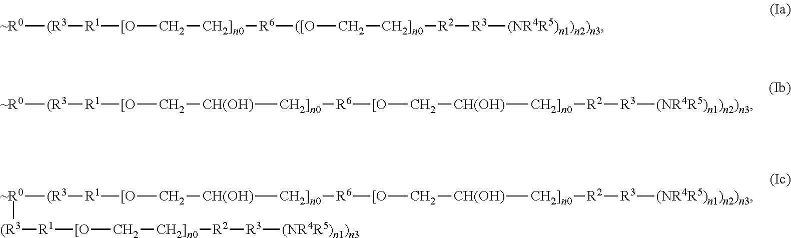

or a stereoisomer or salt thereof, wherein:
   R⁰ is a functional group for attachment to a subunit of the Amino Acid unit;
   each R¹ and R² are independently a bond or C₁-C₆ alkylene;
   each R³ is independently selected from a bond, C₁-C₁₂ alkylene, —C(O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —C₁-C₁₂ alkylene-NR^{a}—C(O)—, —C₁-C₁₂ alkylene-C(O)—NR^{a}—C₁-C₁₂ alkylene-, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, —NR^{a}—C(O)—C₁-C₁₂ alkylene-C(O)—, —C(O)—NR^{a}—C₁-C₁₂ alkylene-(CH(OH))₁₋₈—C₁-C₁₂ alkylene-, —O—CH₂—CH₂, —O—C(O)—NR^{a}—C₁-C₁₂ alkylene, —O—CH₂—CH(OH)—C(O)—, —O—CH₂—CH(OH)—C(O)—NR^{a}—C₁-C₁₂ alkylene-, —CH(OH)—, —CH(OH)—C₁-C₁₂ alkylene-, C₁-C₁₂ alkylene-CH(OH)—, —CH(OH)—C(O)—, —CH(OH)—C(O)—NR^{a}—C₁-C₁₂ alkylene-, —CH(OH)—C₁-C₁₂ alkylene-NR^{a}—C(O)—C₁-C₁₂ alkylene-C(O)—NR^{a}—C₁-C₁₂ alkylene-, —NR^{a}—C(O)—C₁-C₁₂ alkylene-C(O)—NR^{a}—C₁-C₁₂ alkylene-, —CH(OH)—NR^{a}—C₁-C₁₂ alkylene-, —[C(O)—(CH₂)₁₋₈—NR^{a}]₁₋₈—, triazolyl, —C₁-C₁₂ alkylene-triazolyl-, and —C(O)NR⁷R⁸, wherein one of R⁷ and R⁸ is H or C₁-C₁₂ alkylene and the other is C₁-C₁₂ alkylene, each R^{a} is independently selected from H, C₁₋₆ alkyl, and wherein any of the above alkylene groups may be substituted with —SO₃H;
   each R⁴ and R⁵ are independently H, a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
   each R⁶ is independently a bond or selected from:

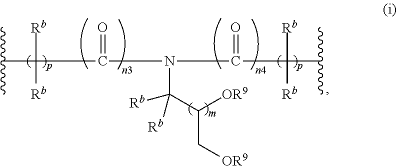

wherein:
   each n³ and n⁴ are independently 0-1,
   each R is independently H or C₁₋₆ alkyl,
   each R⁹ is independently H, acetyl, —P(═O)(OH)₂, or —(CH₂)ᵥ—O—S(═O)₂(OH),
   each p is independently 0-6,
   m is 1-4, and
   each v is independently 1-6, and
   n² is 1;

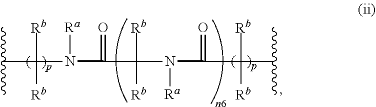

wherein:
each R^{a} is independently H or C₁₋₆ alkyl,
each R^{b} is independently H or C₁₋₆ alkyl,
n⁶ is 1-10, and
each p is independently 0-6, and
n² is 1;

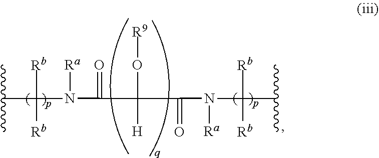

wherein:
each R^{a} is independently H or C₁₋₆ alkyl,
each R^{b} is independently H or C₁₋₆ alkyl,
each R⁹ is independently H, acetyl, —P(═O)(OH)₂, or —(CH₂)ᵥ—O—S(═O)₂(OH),
each p is independently 0-6, and
q is 1-8,
each v is independently 1-6, and
n² is 1;

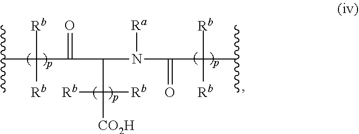

wherein:
each R^{a} is independently H or C₁₋₆ alkyl,
each R^{b} is independently H or C₁₋₆ alkyl, and
each p is independently 0-6, and
n² is 1;
(v) —R¹⁰—[O—CH₂—CH₂]₁₋₈—R¹⁰—, wherein:
each R is independently H or C₁₋₆ alkyl,
each R¹⁰ is independently

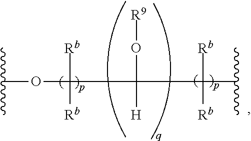

each p is independently 1-6, and
q is 1-8; and
(vi) —N—(R¹—X—R²—[O—CH₂—CH₂]ₙ₀—R²—R³—(NR⁴R⁵)ₙ₁)₂, wherein:
each X is independently —NR^{a}—C(O)— or —C(O)NR^{a}—, and
n² is 2; and
the wavy line (˜) indicates the attachment site of the Amino Acid unit to R⁰;
each n⁰ is independently 2-26;
n¹ is 0-6, and when n¹ is 0 then R³ is —OH or —C(O)OR^{b}, wherein R^{b} is independently H or C₁₋₆ alkyl; and
n³ is 1-6.

In some embodiments, provided is a Linker compound, wherein at least one Polar group attached to the Amino Acid unit comprises the formula:

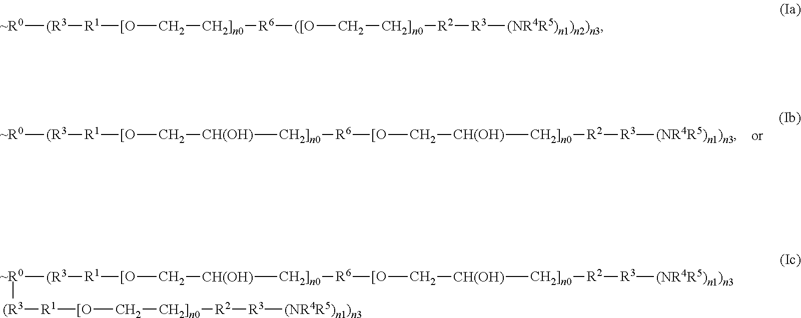

or a stereoisomer or salt thereof, wherein:
   R⁰ is a functional group for attachment to a subunit of the Amino Acid unit;
   each R¹ and R² are independently a bond or C₁-C₆ alkylene;
   each R³ is independently selected from a bond, C₁-C₁₂ alkylene, —C(O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —C₁-C₁₂ alkylene-NR^{a}—C(O)—, —C₁-C₁₂ alkylene-C(O)—NR^{a}—C₁-C₁₂ alkylene-, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, —NR^{a}—C(O)—C₁-C₁₂ alkylene-C(O)—, —C(O)—NR^{a}—C₁-C₁₂ alkylene-(CH(OH))₁₋₈—C₁-C₁₂ alkylene-, —O—CH₂—CH₂, —O—C(O)—NR^{a}—C₁-C₁₂ alkylene, —O—CH₂—CH(OH)—C(O)—, —O—CH₂—CH(OH)—C(O)—NR^{a}—C₁-C₁₂ alkylene-, —CH(OH)—, —CH(OH)—C₁-C₁₂ alkylene-, C₁-C₁₂ alkylene-CH(OH)—, —CH(OH)—C(O)—, —CH(OH)—C(O)—NR^{a}—C₁-C₁₂ alkylene-, —CH(OH)—C₁-C₁₂ alkylene-NR^{a}—C(O)—C₁-C₁₂ alkylene-C(O)—NR^{a}—C₁-C₁₂ alkylene-, —NR^{a}—C(O)—C₁-C₁₂ alkylene-C(O)—NR^{a}—C₁-C₁₂ alkylene-, —CH(OH)—NR^{a}—C₁-C₁₂ alkylene-, —[C(O)—(CH₂)₁₋₈—NR^{a}]₁₋₈—, triazolyl, —C₁-C₁₂ alkylene-triazolyl-, and —C(O)NR⁷R⁸, wherein one of R⁷ and R⁸ is H or C₁-C₁₂ alkylene and the other is C₁-C₁₂ alkylene, each R^{a} is independently selected from H, C₁₋₆ alkyl, and wherein any of the above alkylene groups may be substituted with —SO₃H;
   each R⁴ and R⁵ are independently H, a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, optionally substituted C₃-C₁₀ carbocycle, optionally substituted C₁-C₃ alkylene C₃-C₁₀ carbocycle, optionally substituted heteroaryl, optionally substituted carbocycle, substituted —C₁-C₈ alkyl, substituted —C(O)—C₁-C₈ alkyl, —C(O)—R, where R is a Sugar unit of formula (XII) or (XIII), a chelator, or —NR⁴R⁵ join together to form a C₃-C₈ heterocycle, wherein optional substituents are selected from sulfate, phosphate, alkyl sulfate, and alkyl phosphate, and wherein at least one of R⁴ and R⁵ is not H;
   each R⁶ is independently a bond or selected from:

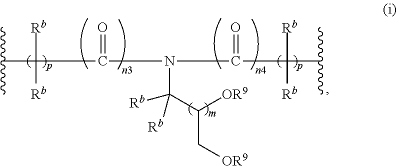

wherein:
   each n³ and n⁴ are independently 0-1,
   each R is independently H or C₁₋₆ alkyl,
   each R⁹ is independently H, acetyl, —P(═O)(OH)₂, or —(CH₂)ᵥ—O—S(═O)₂(OH),
   each p is independently 0-6,
   m is 1-4, and
   each v is independently 1-6, and
   n² is 1;

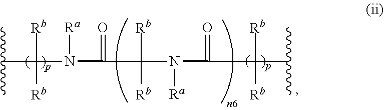

wherein:
   each R^{a} is independently H or C₁₋₆ alkyl,
   each R^{b} is independently H or C₁₋₆ alkyl,
   n⁶ is 1-10, and
   each p is independently 0-6, and
   n² is 1;

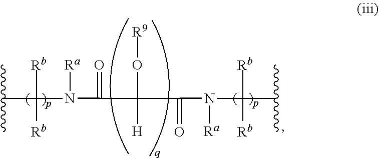

wherein:
   each R^{a} is independently H or C₁₋₆ alkyl,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6, and
   q is 1-8,
   each v is independently 1-6, and
   n² is 1;

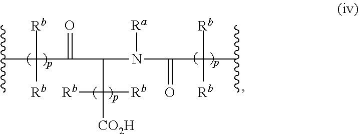

wherein:
   each R^{a} is independently H or C₁₋₆ alkyl,
   each R^{b} is independently H or C₁₋₆ alkyl, and
   each p is independently 0-6, and
   n² is 1;
   (v) —R¹⁰—[O—CH₂—CH₂]₁₋₈—R¹⁰—, wherein:
   each R^{b} is independently H or C₁₋₆ alkyl,
   each R¹⁰ is independently

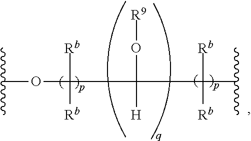

each p is independently 1-6, and
q is 1-8; and
(vi) —N—(R¹—X—R²—[O—CH₂—CH₂]ₙ₀—R²—R³—(NR⁴R⁵)ₙ₁)₂, wherein:
each X is independently —NR^{a}—C(O)— or —C(O)NR^{a}—, and
n² is 2; and
the wavy line (˜) indicates the attachment site of the Amino Acid unit to R⁰;
each n⁰ is independently 2-26;
n¹ is 0-6, and when n¹ is 0 then R³ is —OH or —C(O)OR^{b}, wherein R is independently H or C₁₋₆ alkyl; and
n³ is 1-6.

In some embodiments, provided is a Linker compound, wherein each R³ is independently selected from a bond, —C(O)—, —NR^{a}—C(O)—C₁-C₁₂ alkylene-C(O)—, —C(O)—NR^{a}—C₁-C₁₂ alkylene-(CH(OH))₁₋₈—C₁-C₁₂ alkylene-, —O—CH₂—CH(OH)—C(O)—, —O—CH₂—CH(OH)—C(O)—NR^{a}—C₁-C₁₂ alkylene-, —CH(OH)—, —CH(OH)—C₁-C₁₂ alkylene-, C₁-C₁₂ alkylene-CH(OH)—, —CH(OH)—C(O)—, —CH(OH)—C(O)—NR^{a}—C₁-C₁₂ alkylene-, —CH(OH)—C₁-C₁₂ alkylene-NR^{a}—C(O)—C₁-C₁₂ alkylene-C(O)—NR^{a}—C₁-C₁₂ alkylene-, —NR^{a}—C(O)—C₁-C₁₂ alkylene-C(O)—NR^{a}—C₁-C₁₂ alkylene-, —CH(OH)—NR^{a}—C₁-C₁₂ alkylene-, —[C(O)—(CH₂)₁₋₈—NR^{a}]₁₋₈—, triazolyl, and —C₁-C₁₂ alkylene-triazolyl-, —N(polyhydroxyl group)-, each R^{a} is independently selected from H, C₁₋₆ alkyl; and wherein any of the above alkylene groups may be substituted with —SO₃H.

In some embodiments, provided is a Linker compound, wherein each R³ is independently selected from a bond, —C(O)—, —NR^{a}—C(O)—C₁-C₁₂ alkylene-C(O)—, —C(O)—NR^{a}—C₁-C₁₂ alkylene-(CH(OH))₁₋₈—C₁-C₁₂ alkylene-, —O—CH₂—CH(OH)—C(O)—, —O—CH₂—CH(OH)—C(O)—NR^{a}—C₁-C₁₂ alkylene-, C₁-C₁₂ alkylene-CH(OH)—, —CH(OH)—C(O)—, —CH(OH)—C(O)—NR^{a}—C₁-C₁₂ alkylene-, —CH(OH)—C₁-C₁₂ alkylene-NR^{a}—C(O)—C₁-C₁₂ alkylene-C(O)—NR^{a}—C₁-C₁₂ alkylene-, —NR^{a}—C(O)—C₁-C₁₂ alkylene-C(O)—NR^{a}—C₁-C₁₂ alkylene-, —[C(O)—(CH₂)₁₋₈—NR^{a}]₁₋₈, triazolyl, and —C₁-C₁₂ alkylene-triazolyl-, —N(polyhydroxyl group)-, each R^{a} is independently selected from H, C₁₋₆ alkyl; and wherein any of the above alkylene groups may be substituted with —SO₃H.

In some embodiments, provided is a Linker compound, wherein the Linker unit comprises a moiety selected from:

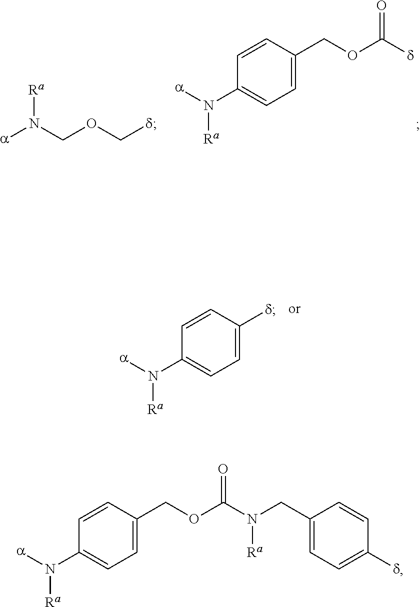

or a stereoisomer or salt thereof, wherein:
   α—represents a direct or indirect attachment site to the Amino Acid unit;
   d—represents an attachment site to at least one of the Drug units or for an attachment site to a linking group attached to the at least one of the Drug units; and
   R^{a} is H or C₁₋₆ alkyl.

In some embodiments, provided is a Linker compound, wherein the at least one Polar group comprises at least one Sugar unit having the following formula:

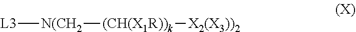

or a stereoisomer or salt thereof, wherein:
   each X₁ is independently selected from NH or O;
   each R is independently selected from hydrogen, acetyl, a monosaccharide, a disaccharide, and a polysaccharide;
   each X₂ is independently selected from CH₂ and C(O);
   each X₃ is independently selected from H, OH and OR;
   k is 1 to 10; and
L3 is a point of attachment to the remainder of the Polar group.

In some embodiments, provided is a Linker compound, wherein the at least one Polar group comprises at least one Sugar unit having one of the following structures (XII) or (XIII):

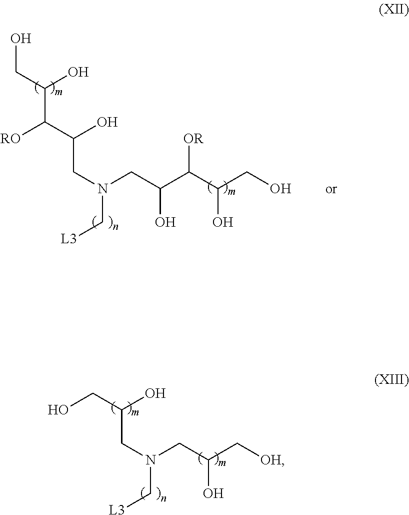

or a stereoisomer or salt thereof, wherein:
   each R is independently selected from hydrogen, a monosaccharide, a disaccharide and a polysaccharide;
   m is 1 to 8; and
   n is 0 to 4.

In some embodiments, provided is a Linker compound, comprising a Polar group having a formula selected from:

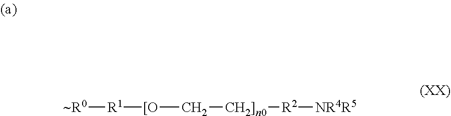

or a stereoisomer or salt thereof, wherein:
   R⁰ is a functional group for attachment to a subunit of the Amino Acid unit;
   R¹ and R² are each, independently, a bond or C₁-C₃ alkylene;
   R⁴ and R⁵ are each independently selected from a H, polyhydroxyl group, substituted polyhydroxyl group, —C(O)-polyhydroxyl group, substituted —C(O)-polyhydroxyl group, a chelator, and —C(O)—R, where R is a Sugar unit of formula (XII) or (XIII), wherein both R⁴ and R⁵ are not H; and
n0 is 2 to 26;

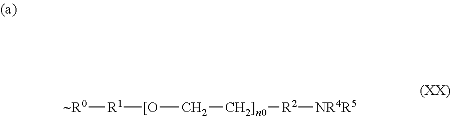

or a stereoisomer or salt thereof, wherein:
   R⁰ is a functional group for attachment to a subunit of the Amino Acid unit;
   R¹ and R² are each, independently, a bond or C₁-C₃ alkylene;
   one of R⁴ and R⁵ is selected from a H, polyhydroxyl group, substituted polyhydroxyl group, —C(O)-polyhydroxyl group, substituted —C(O)-polyhydroxyl group, a chelator, and —C(O)—R, where R is a Sugar unit of formula (XII) or (XIII), and the other of R⁴ and R⁵ is a polyethylene glycol, optionally having 1 to 24 ethylene glycol subunits, wherein both R⁴ and R⁵ are not H; and
   n0 is 2 to 26;

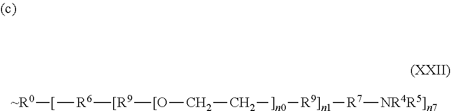

or a stereoisomer or salt thereof, wherein:
   R⁰ is a functional group for attachment to a subunit of the Amino Acid unit;
   R⁶ and R are each, independently, selected from a bond, C₁-C₁₂ alkylene, —NH—C₁-C₁₂ alkylene, —C₁-C₁₂ alkylene-NH—, —C₁-C₁₂ alkylene-N(CH₃)—, —C(O)—C₁-C₁₂ alkylene, —C₁-C₁₂ alkylene-C(O)—, —NH—C₁-C₁₂ alkylene-C(O)— and —C(O)—C₁-C₁₂ alkylene-NH—;
   one of R⁴ and R⁵ is selected from a H, polyhydroxyl group, substituted polyhydroxyl group, —C(O)-polyhydroxyl group, substituted —C(O)-polyhydroxyl group, a chelator, and —C(O)—R, where R is a Sugar unit of formula (XII) or (XIII); and the other of R⁴ and R⁵ is selected from H, polyhydroxyl group, substituted polyhydroxyl group, —C(O)-polyhydroxyl group, substituted —C(O)-polyhydroxyl group, a chelator, and —C(O)—R, where R is a Sugar unit of formula (XII) or (XIII), and polyethylene glycol, optionally having 1 to 24 ethylene glycol subunits, wherein both R⁴ and R⁵ are not H;
   each R⁹ is independently selected from a bond, —C(O)—, —NH—, —C(O)—C₁-C₆ alkylene-, —NH—C₁-C₆ alkylene-, —C₁-C₆ alkylene-NH—, —C₁-C₆ alkylene-C(O)—, —NH(CO)—C₁-C₆alkylene-, —N(CH₃)—(CO)—C₁-C₆alkylene-, —NH(CO)NH—, and triazole;
   n0 is 2 to 26;
   n1 is 1 to 4; and
   n7 is 1 to 4;

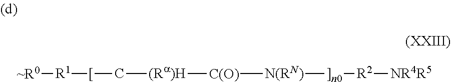

or a stereoisomer or salt thereof, wherein:
   R⁰ is a functional group for attachment to a subunit of the Amino Acid unit;
   R¹ is a bond, C₁-C₃ alkylene, —C₁-C₃alkylene-[O—CH₂—CH₂-]ₙ₀, —[CH₂—CH₂—O]ₙ₀—C₁-C₃alkylene- or —C₁-C₃ alkylene-[O—CH₂—CH₂—]ₙ₀—C(O)—; R² is C₁-C₃ alkylene, —C₁-C₃alkylene-[O—CH₂—CH₂-]ₙ₀, —[CH₂—CH₂—O]ₙ₀—C₁-C₃alkylene- or —C₁-C₃ alkylene-[O—CH₂—CH₂—]ₙ₀—C(O)—;
   each R^{α} is independently H or —R²—NR⁴R⁵;
   each R^{N} is independently H, C₁-C₆ alkyl or —R²—NR⁴R⁵;
   R⁴ and R⁵ are each independently selected from a H, polyhydroxyl group, substituted polyhydroxyl group, —C(O)-polyhydroxyl group, substituted —C(O)-polyhydroxyl group, a chelator, and —C(O)—R, where R is a Sugar unit of formula (XII) or (XIII), wherein both R⁴ and R⁵ are not H; and
   each n0 is independently 2 to 26;

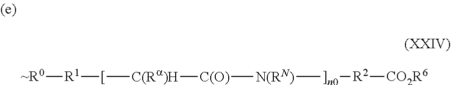

or a stereoisomer or salt thereof, wherein:
   R⁰ is a functional group for attachment to a subunit of the Amino Acid unit;
   R¹ is a bond, C₁-C₃ alkylene, or —C₁-C₃ alkylene[O—CH₂—CH₂—]ₙ₀;
   R² is C₁-C₃ alkylene, or —C₁-C₃ alkylene[O—CH₂—CH₂—]ₙ₀;
   each R^{α} is independently H or —R²—NR⁴R⁵;
   each R^{N} is independently H, C₁-C₆ alkyl or —R²—NR⁴R⁵;
   R⁴ and R⁵ are each independently selected from a H, polyhydroxyl group, substituted polyhydroxyl group, —C(O)-polyhydroxyl group, substituted —C(O)-polyhydroxyl group, a chelator, and —C(O)—R, where R is a Sugar unit of formula (XII) or (XIII), wherein both R⁴ and R⁵ are not H;
   R⁶ is H or C₁-C₄ alkyl; and
   each n0 is independently 2 to 26,
   with the proviso that at least one R^{α} or R^{N} is —R²—NR⁴R⁵; or

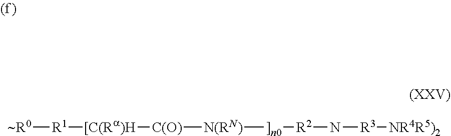

or a stereoisomer or salt thereof, wherein:
   R⁰ is a functional group for attachment to a subunit of the Amino Acid unit;
   R¹ and R² are each, independently, a bond, C₁-C₃ alkylene, or
   —C₁-C₃alkylene-[O—CH₂—CH₂—]ₙ₀;
   each R^{α} is independently H or —R²—NR⁴R⁵;
   each R^{N} is independently H or C₁-C₆ alkyl;
   each R³ is independently C₁-C₆ alkylene;
   R⁴ and R⁵ are each independently selected from a H, polyhydroxyl group, substituted polyhydroxyl group, —C(O)-polyhydroxyl group, substituted —C(O)-polyhydroxyl group, a chelator, and —C(O)—R, where R is a Sugar unit of formula (XII) or (XIII), wherein both R⁴ and R⁵ are not H; and
   each n0 is independently 2 to 26.

In some embodiments, provided is a Linker compound, comprising a Polar group having a formula selected from:

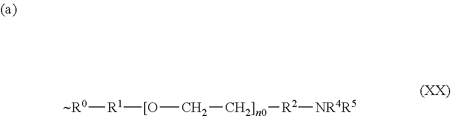

or a stereoisomer or salt thereof, wherein:
   R⁰ is a functional group for attachment to a subunit of the Amino Acid unit;
   R¹ and R² are each, independently, a bond or C₁-C₃ alkylene;
   R⁴ and R⁵ are each independently H, a polyhydroxyl group, a substituted polyhydroxyl group, a —C(O)-polyhydroxyl group, a substituted —C(O)-polyhydroxyl group, a polyhydroxyl-ether group, a substituted polyhydroxyl-ether group, optionally substituted C₃-C₁₀ carbocycle, optionally substituted C₁-C₃ alkylene C₃-C₁₀ carbocycle, optionally substituted heteroaryl, optionally substituted carbocycle, substituted —C₁-C₈ alkyl, substituted —C(O)—C₁-C₈ alkyl, —C(O)—R, where R is a Sugar unit of formula (XII) or (XIII), and a chelator, or —NR⁴R⁵ join together to form a C₃-C₈ heterocycle, wherein optional substituents are selected from sulfate, phosphate, alkyl sulfate, and alkyl phosphate, and wherein at least one of R⁴ and R⁵ is not H;
   n0 is 2 to 26;

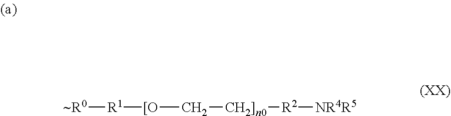

or a stereoisomer or salt thereof, wherein:
   R⁰ is a functional group for attachment to a subunit of the Amino Acid unit;
   R¹ and R² are each, independently, a bond or C₁-C₃ alkylene;
   one of R⁴ and R⁵ is selected from a H, polyhydroxyl group, substituted polyhydroxyl group, —C(O)-polyhydroxyl group, substituted —C(O)-polyhydroxyl group, a chelator, and —C(O)—R, where R is a Sugar unit of formula (XII) or (XIII), and the other of R⁴ and R⁵ is a polyethylene glycol, optionally having 1 to 24 ethylene glycol subunits, wherein both R⁴ and R⁵ are not H; and
   n0 is 2 to 26;

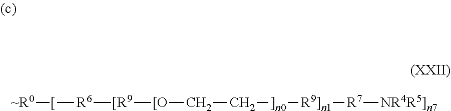

or a stereoisomer or salt thereof, wherein:
   R⁰ is a functional group for attachment to a subunit of the Amino Acid unit;
   R⁶ and R⁷ are each, independently, selected from a bond, C₁-C₁₂ alkylene, —NH—C₁-C₁₂ alkylene, —C₁-C₁₂ alkylene-NH—, —C₁-C₁₂ alkylene-N(CH₃)—, —C(O)—C₁-C₁₂ alkylene, —C₁-C₁₂ alkylene-C(O)—, —NH—C₁-C₁₂ alkylene-C(O)— and —C(O)—C₁-C₁₂ alkylene-NH—;
   one of R⁴ and R⁵ is selected from a H, polyhydroxyl group, substituted polyhydroxyl group, —C(O)-polyhydroxyl group, substituted —C(O)-polyhydroxyl group, a chelator, and —C(O)—R, where R is a Sugar unit of formula (XII) or (XIII); and the other of R⁴ and R⁵ is selected from H, polyhydroxyl group, substituted polyhydroxyl group, —C(O)-polyhydroxyl group, substituted —C(O)-polyhydroxyl group, a chelator, and —C(O)—R, where R is a Sugar unit of formula (XII) or (XIII), and polyethylene glycol, optionally having 1 to 24 ethylene glycol subunits, or —NR⁴R⁵ together from a C₃-C₅ heterocycle, wherein both R⁴ and R⁵ are not H;
   each R⁹ is independently selected from a bond, —C(O)—, —NH—, —C(O)—C₁-C₆ alkylene-, —NH—C₁-C₆ alkylene-, —C₁-C₆ alkylene-NH—, —C₁-C₆ alkylene-C(O)—, —NH(CO)—C₁-C₆alkylene-, —N(CH₃)—(CO)—C₁-C₆alkylene-, —NH(CO)NH—, and triazole;
   n0 is 2 to 26;
   n1 is 1 to 4; and
   n7 is 1 to 4;

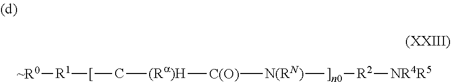

or a stereoisomer or salt thereof, wherein:
   R⁰ is a functional group for attachment to a subunit of the Amino Acid unit;
   R¹ is a bond, C₁-C₃ alkylene, —C₁-C₃alkylene-[O—CH₂—CH₂-]ₙ₀, —[CH₂—CH₂—O]ₙ₀—C₁-C₃alkylene- or —C₁-C₃ alkylene-[O—CH₂—CH₂-]ₙ₀—C(O)—;
   R² is C₁-C₃ alkylene, —C₁-C₃alkylene-[O—CH₂—CH₂—]ₙ₀, —[CH₂—CH₂—O]ₙ₀—C₁-C₃alkylene- or —C₁-C₃ alkylene-[O—CH₂—CH₂—]ₙ₀—C(O)—;
   each R^{α} is independently H or —R²—NR⁴R⁵;
   each R^{N} is independently H, C₁-C₆ alkyl or —R²—NR⁴R⁵;
   R⁴ and R⁵ are each independently H, a polyhydroxyl group, a substituted polyhydroxyl group, a —C(O)-polyhydroxyl group, a substituted —C(O)-polyhydroxyl group, a polyhydroxyl-ether group, a substituted polyhydroxyl-ether group, optionally substituted C₃-C₁₀ carbocycle, optionally substituted C₁-C₃ alkylene C₃-C₁₀ carbocycle, optionally substituted heteroaryl, optionally substituted carbocycle, substituted —C₁-C₈ alkyl, substituted —C(O)—C₁-C₈ alkyl, —C(O)—R, where R is a Sugar unit of formula (XII) or (XIII), and a chelator, or —NR⁴R⁵ join together to form a C₃-C₈ heterocycle, wherein optional substituents are selected from sulfate, phosphate, alkyl sulfate, and alkyl phosphate, and wherein at least one of R⁴ and R⁵ is not H; and
   each n0 is independently 2 to 26;

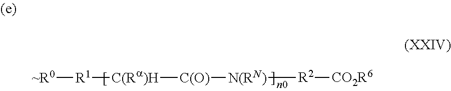

or a stereoisomer or salt thereof, wherein:
   R⁰ is a functional group for attachment to a subunit of the Amino Acid unit;
   R¹ is a bond, C₁-C₃ alkylene, or —C₁-C₃ alkylene[O—CH₂—CH₂-]ₙ₀;
   R² is C₁-C₃ alkylene, or —C₁-C₃ alkylene[O—CH₂—CH₂-]ₙ₀;
   each R^{α} is independently H or —R²—NR⁴R⁵;
   each R^{N} is independently H, C₁-C₆ alkyl or —R²—NR⁴R⁵;
   R⁴ and R⁵ are each independently H, a polyhydroxyl group, a substituted polyhydroxyl group, a —C(O)-polyhydroxyl group, a substituted —C(O)-polyhydroxyl group, a polyhydroxyl-ether group, a substituted polyhydroxyl-ether group, optionally substituted C₃-C₁₀ carbocycle, optionally substituted C₁-C₃ alkylene C₃-C₁₀ carbocycle, optionally substituted heteroaryl, optionally substituted carbocycle, substituted —C₁-C₈ alkyl, substituted —C(O)—C₁-C₈ alkyl, —C(O)—R, where R is a Sugar unit of formula (XII) or (XIII), and a chelator, or —NR⁴R⁵ join together to form a C₃-C₈ heterocycle, wherein optional substituents are selected from sulfate, phosphate, alkyl sulfate, and alkyl phosphate, and wherein at least one of R⁴ and R⁵ is not H;
   R⁶ is H or C₁-C₄ alkyl; and
   each n0 is independently 2 to 26,
   with the proviso that at least one R^{α} or R^{N} is —R²—NR⁴R⁵; or

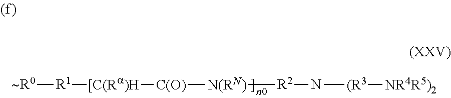

or a stereoisomer or salt thereof, wherein:
   R⁰ is a functional group for attachment to a subunit of the Amino Acid unit;
   R¹ and R² are each, independently, a bond, C₁-C₃ alkylene, or
   —C₁-C₃alkylene-[O—CH₂—CH₂—]ₙ₀;
   each R^{α} is independently H or —R²—NR⁴R⁵;
   each R^{N} is independently H or C₁-C₆ alkyl;
   each R³ is independently C₁-C₆ alkylene;
   R⁴ and R⁵ are each independently H, a polyhydroxyl group, a substituted polyhydroxyl group, a —C(O)-polyhydroxyl group, a substituted —C(O)-polyhydroxyl group, a polyhydroxyl-ether group, a substituted polyhydroxyl-ether group, optionally substituted C₃-C₁₀ carbocycle, optionally substituted C₁-C₃ alkylene C₃-C₁₀ carbocycle, optionally substituted heteroaryl, optionally substituted carbocycle, substituted —C₁-C₈ alkyl, substituted —C(O)—C₁-C₈ alkyl, —C(O)—R, where R is a Sugar unit of formula (XII) or (XIII), and a chelator, or —NR⁴R⁵ join together to form a C₃-C₈ heterocycle, wherein optional substituents are selected from sulfate, phosphate, alkyl sulfate, and alkyl phosphate, and wherein at least one of R⁴ and R⁵ is not H; and
   each n0 is independently 2 to 26.

In some embodiments, provided is a Linker compound, wherein R⁴ and R⁵ are each independently selected from H and polyhydroxyl group, and wherein at least one of R⁴ and R⁵ is not H.

In some embodiments, provided is a Linker compound, wherein the polyhydroxyl group is a linear monosaccharide, optionally selected from a C6 or C5 sugar, sugar acid or amino sugar.

In some embodiments, provided is a Linker compound, wherein:
the C6 or C5 sugar is selected from glucose, ribose, galactose, mannose, arabinose, 2-deoxyglucose, glyceraldehyde, erythrose, threose, xylose, lyxose, allose, altrose, gulose, idose, talose, aldose, and ketose;
the sugar acid is selected from gluconic acid, aldonic acid, uronic acid and ulosonic acid; or
the amino sugar is selected from glucosamine, N-acetyl glucosamine, galactosamine, and N-acetyl galactosamine.

In some embodiments, provided is a Linker compound, comprising a Polar group selected from the following, or a stereoisomer or salt thereof:

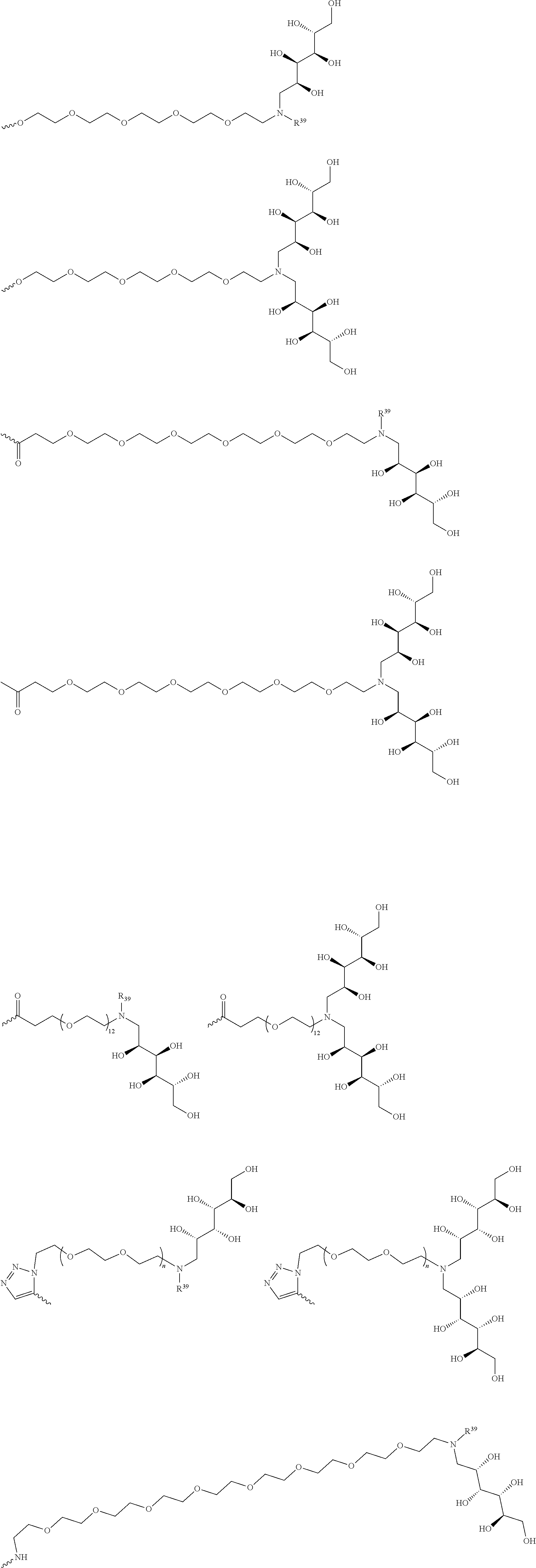

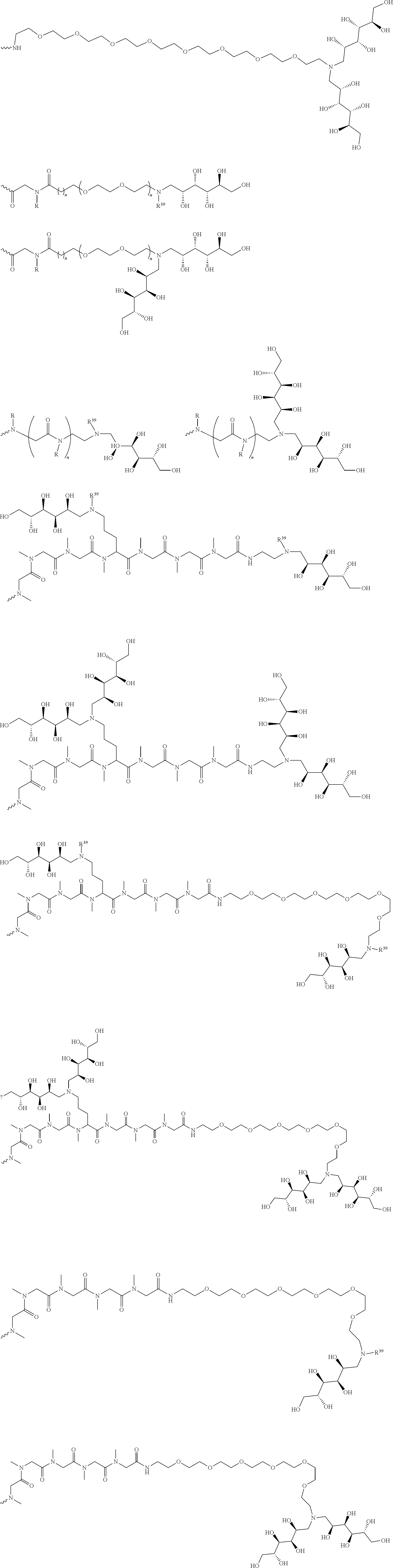

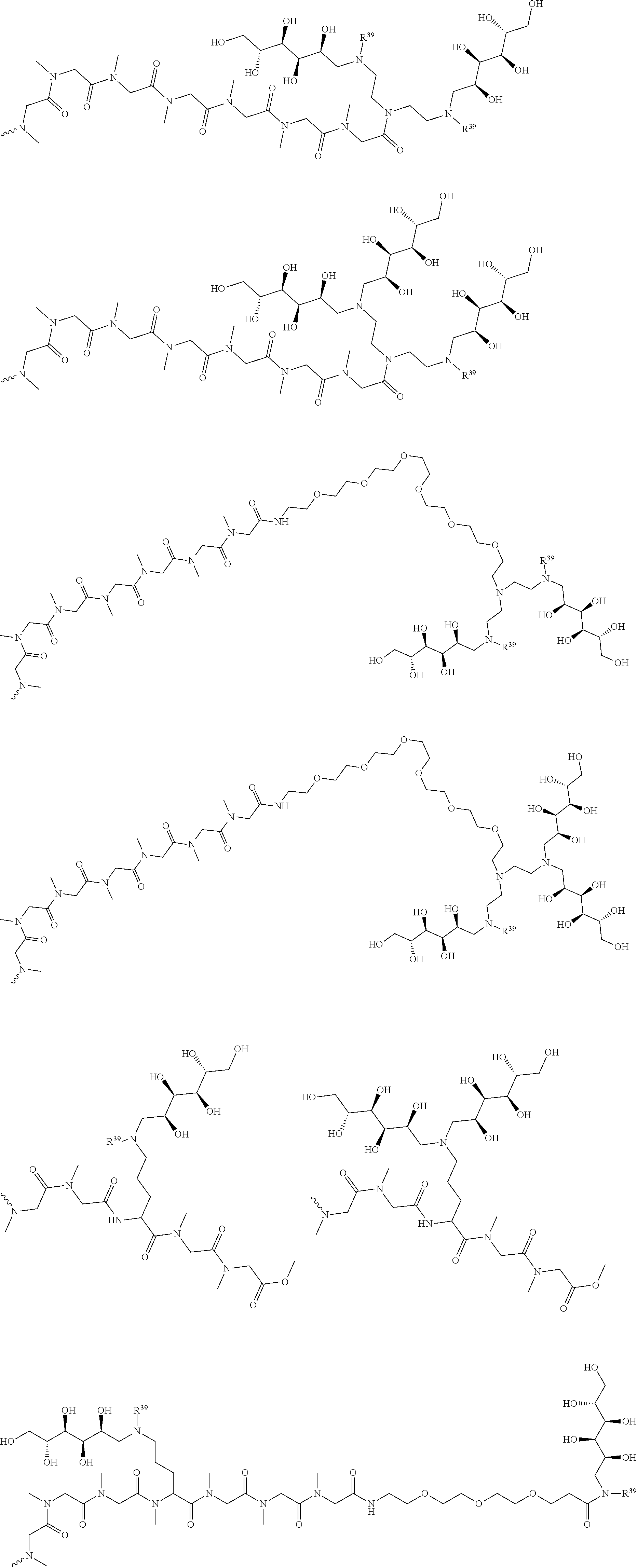

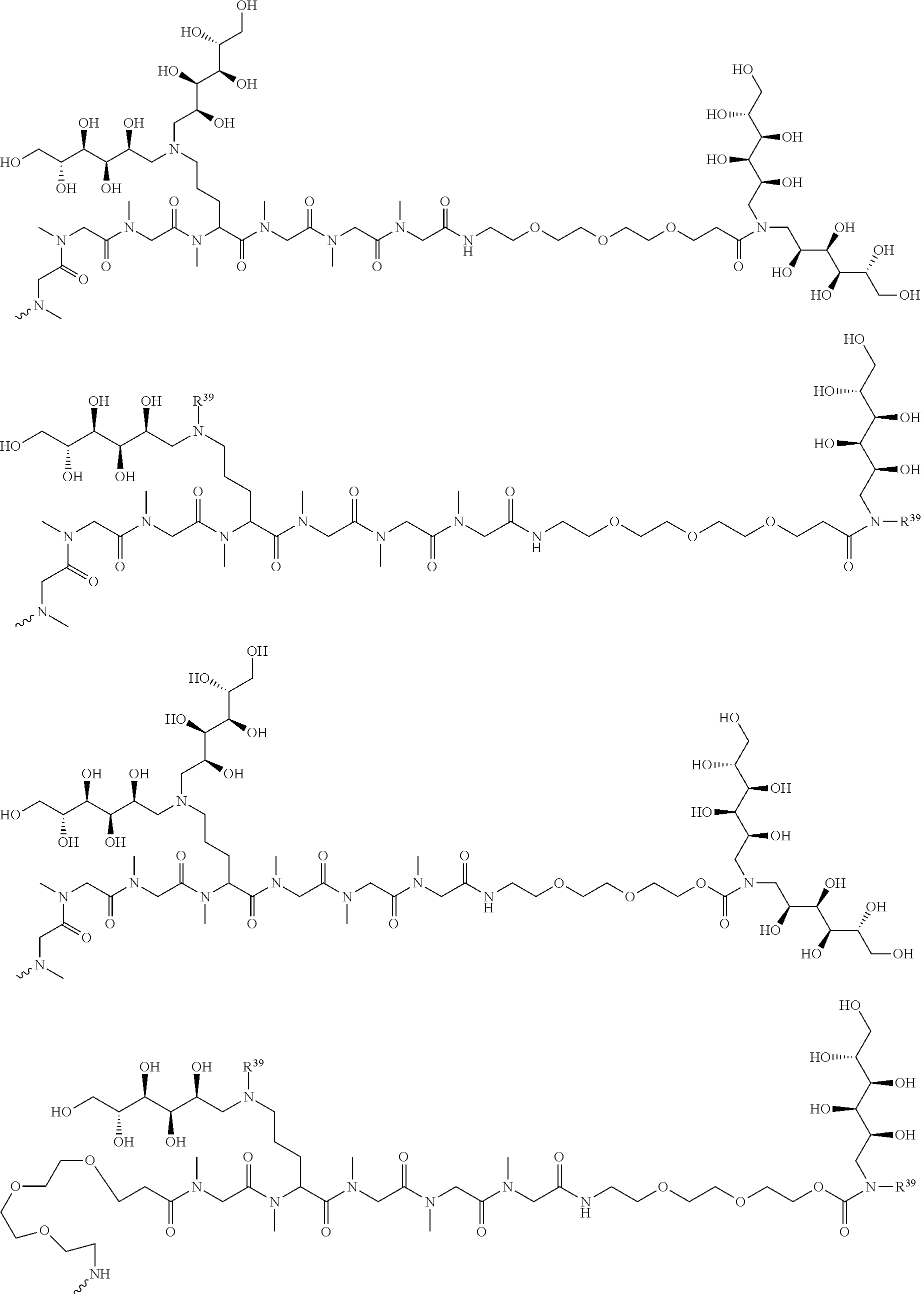

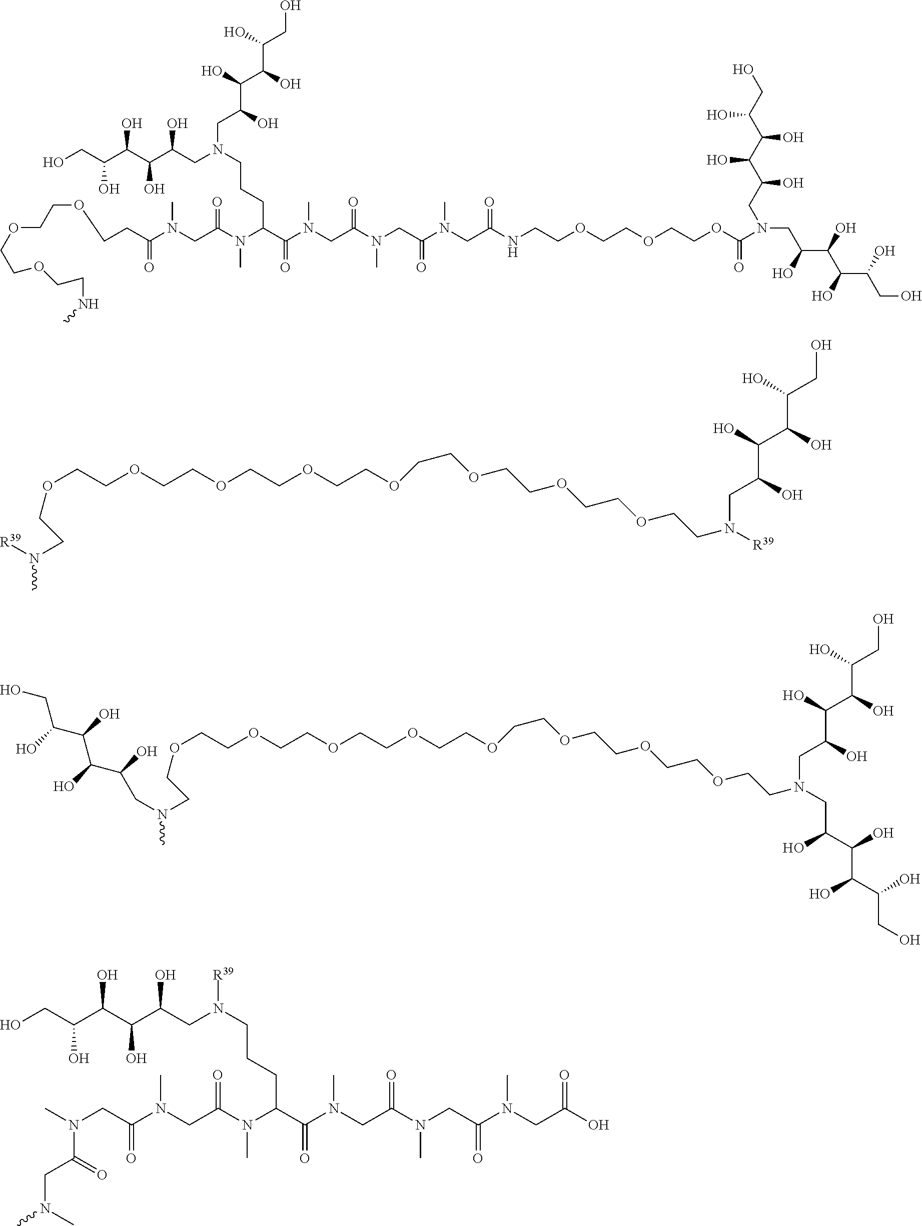

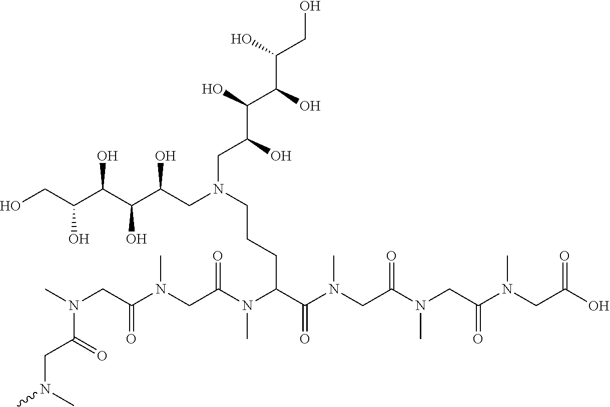

wherein each R is independently H or alkyl; each R³⁹ is independently selected from H, a linear monosaccharide and polyethylene glycol, optionally having from 1 to 24 ethylene glycol subunits; each n independently is 1-12; and the wavy line is an attachment to the Amino Acid unit.

In some embodiments, provided is a Linker compound, wherein one of R⁴ and R⁵ is a linear monosaccharide and the other is a cyclic monosaccharide.

In some embodiments, provided is a Linker compound, wherein —(NR⁴R⁵) is selected from the following, or a stereoisomer or salt thereof:

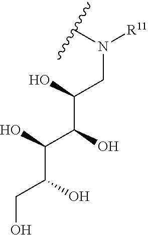

wherein R¹¹ is a cyclic monosaccharide.

In some embodiments, provided is a Linker compound, comprising a Polar group selected from the following, or a stereoisomer or salt thereof:

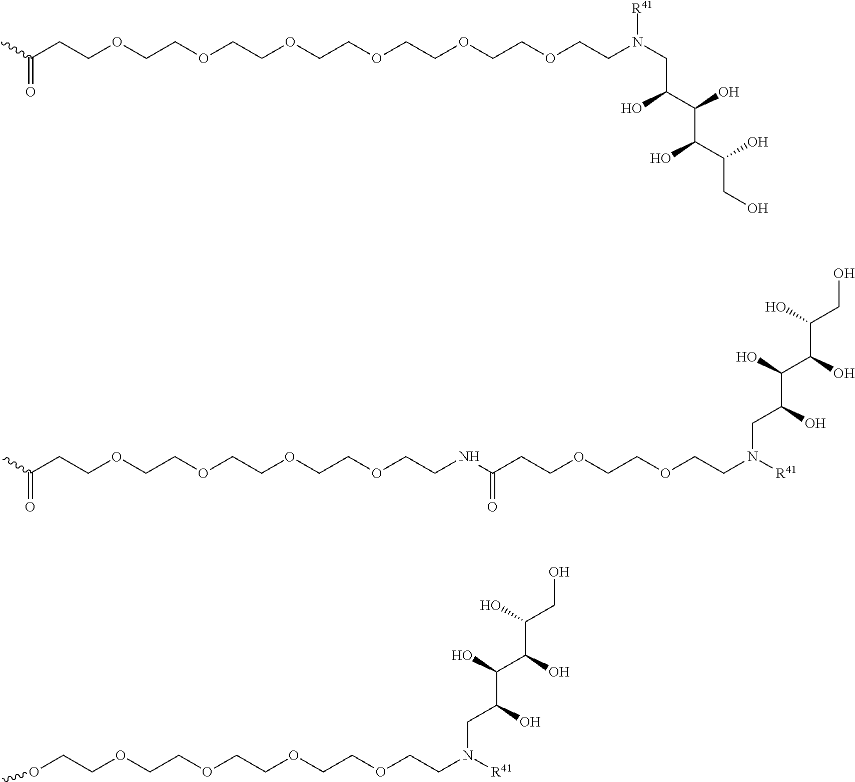

wherein R⁴¹ is a cyclic monosaccharide; and the wavy line is an attachment to the Amino Acid unit.

In some embodiments, provided is a Linker compound, wherein R⁴ and R⁵ are independently a polyhydroxyl selected from a cyclic monosaccharide, disaccharide and polysaccharide.

In some embodiments, provided is a Linker compound, wherein —(NR⁴R⁵) is selected from the following, or a stereoisomer or salt thereof:

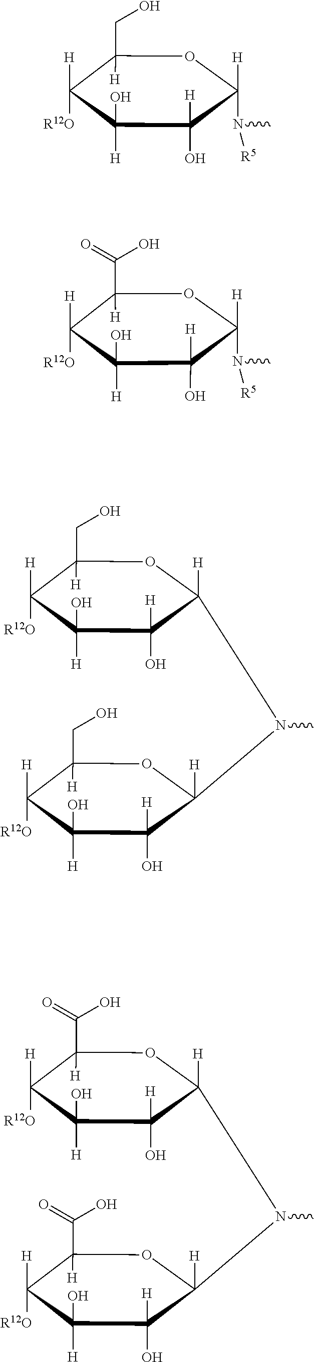

wherein each R¹² is selected from H and a monosaccharide, a disaccharide, or a polysaccharide; and R⁵ is selected from a cyclic monosaccharide, disaccharide, or polysaccharide.

In some embodiments, provided is a Linker compound, comprising a Polar group selected from the following, or a stereoisomer or salt thereof:

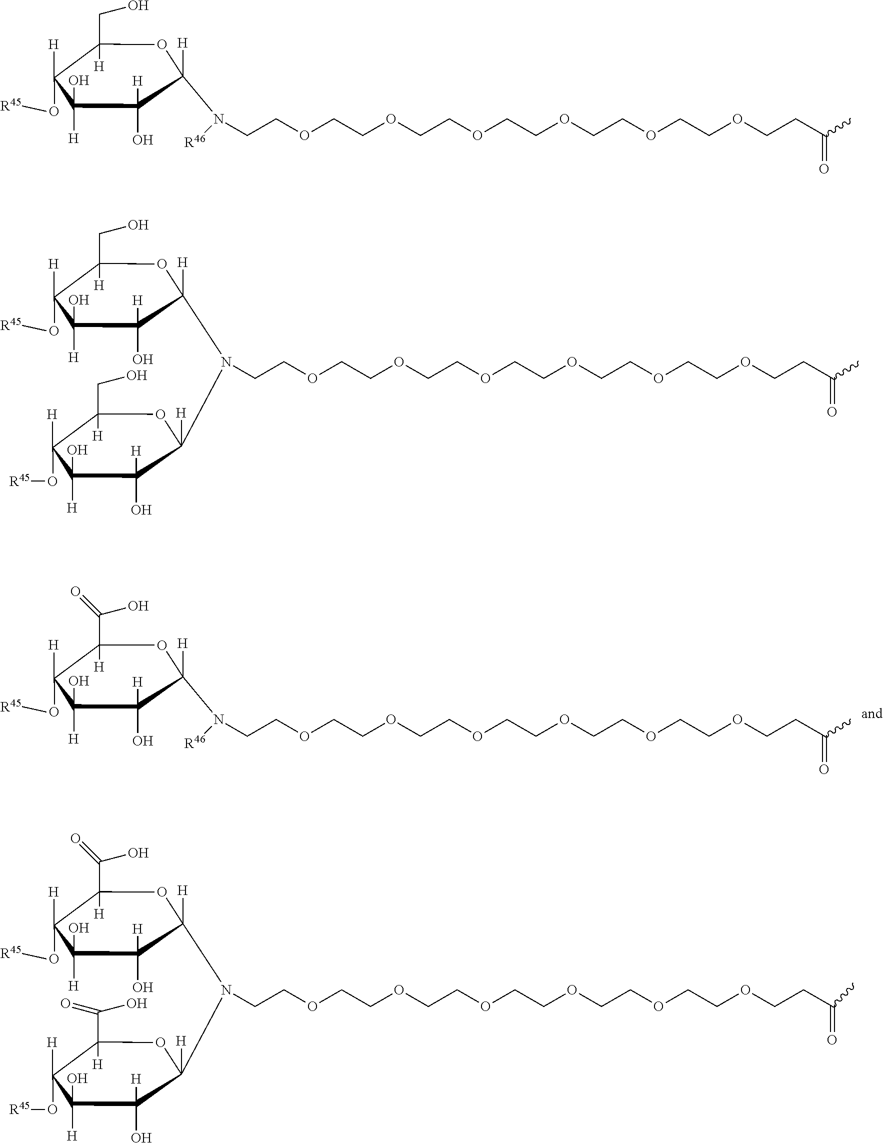

wherein each R⁴⁵ is selected from H and a monosaccharide, a disaccharide, or a polysaccharide; and R⁴⁶ is selected from a cyclic monosaccharide, disaccharide, or polysaccharide; and the wavy line is an attachment to the Amino Acid unit.

In some embodiments, provided is a Linker compound, wherein R⁴ and R⁵ are independently selected from a linear monosaccharide and a substituted linear monosaccharide, wherein the substituted linear monosaccharide is substituted with a monosaccharide, a disaccharide or a polysaccharide.

In some embodiments, provided is a Linker compound, wherein —(NR⁴R⁵) is selected from the following, or a stereoisomer or salt thereof:

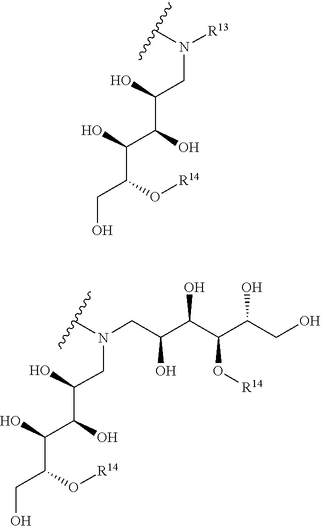

wherein R¹³ is a linear monosaccharide; and each R¹⁴ is selected from a monosaccharide, a disaccharide and a polysaccharide.

In some embodiments, provided is a Linker compound, comprising a Polar group selected from the following, or a stereoisomer or salt thereof:

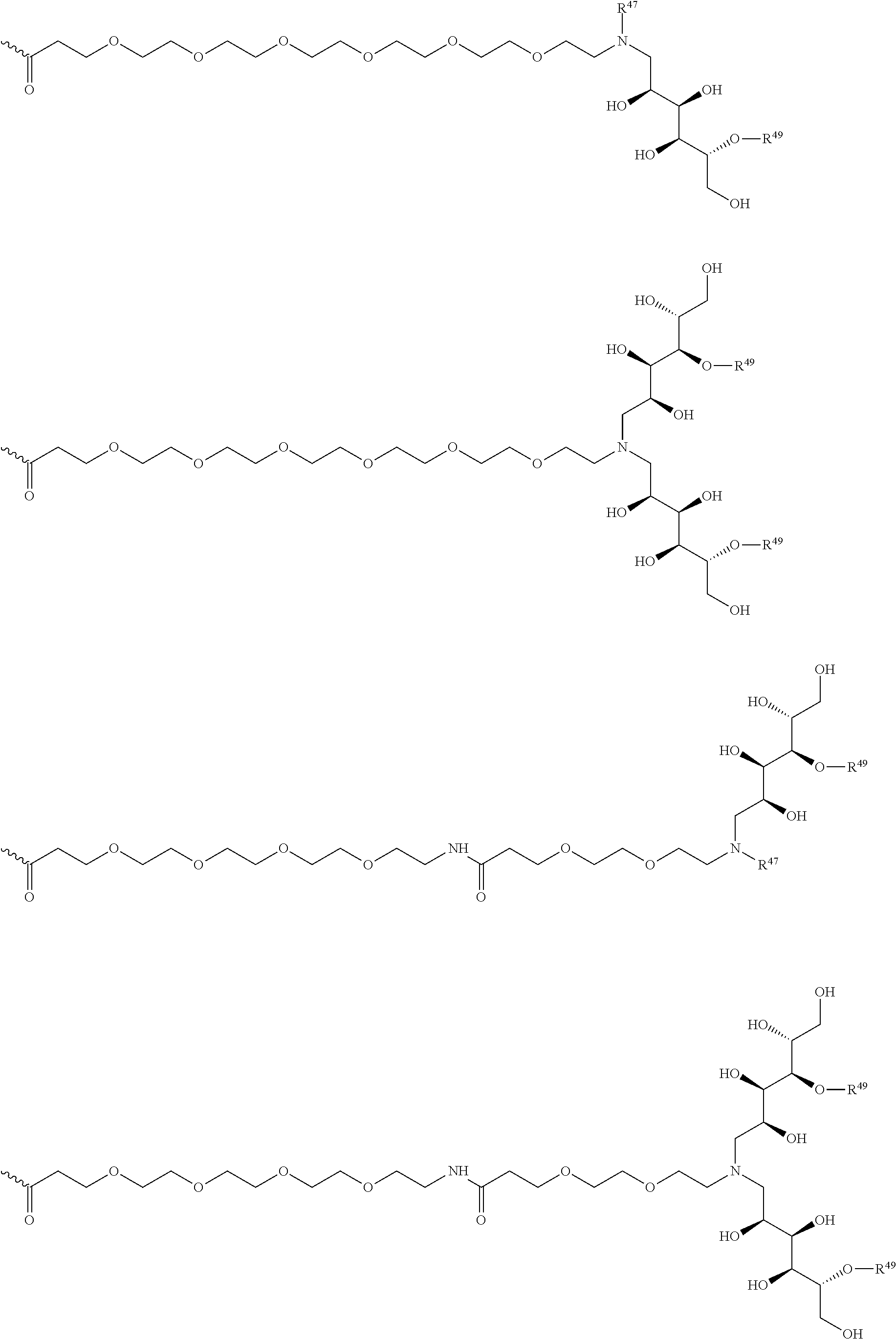

wherein R⁴⁷ is a linear monosaccharide; and each R⁴⁹ is selected from a monosaccharide, a disaccharide and a polysaccharide; and the wavy line is an attachment to the Amino Acid unit.

In some embodiments, provided is a Linker compound, wherein R⁴ and R⁵ are independently selected from a linear monosaccharide and a substituted monosaccharide, wherein the substituted linear monosaccharide is substituted with one or more substituents selected from carboxyl, ester, and amide, and optionally further substituted with a monosaccharide, disaccharide or a polysaccharide.

In some embodiments, provided is a Linker compound, wherein R⁴ and R⁵ are independently selected from a linear monosaccharide and a substituted monosaccharide, wherein the substituted linear monosaccharide is substituted with one or more substituents selected from alkyl, O-alkyl, aryl, O-aryl, carboxyl, ester, or amide, and optionally further substituted with a monosaccharide, disaccharide or a polysaccharide.

In some embodiments, provided is a Linker compound, wherein —(NR⁴R⁵) is selected from the following, or a stereoisomer or salt thereof:

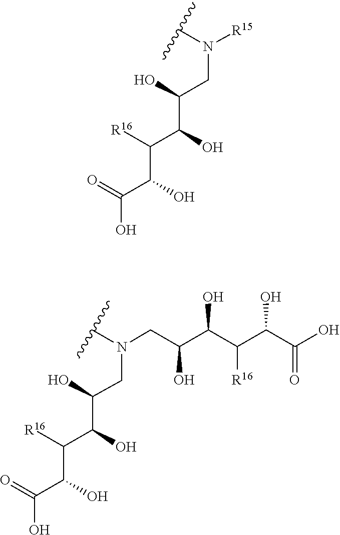

wherein each R¹⁵ is independently selected from a linear monosaccharide and a substituted linear monosaccharide; each R¹⁶ is independently selected from carboxyl, ester, and amide.

In some embodiments, provided is a Linker compound, wherein —(NR⁴R⁵) is selected from the following, or a stereoisomer or salt thereof:

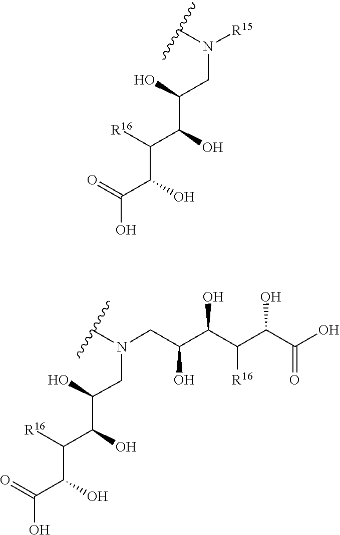

wherein each R¹⁵ is independently selected from a linear monosaccharide and a substituted linear monosaccharide; each R¹⁶ is independently selected from alkyl, O-alkyl, aryl, O-aryl, carboxyl, ester, and amide.

In some embodiments, provided is a Linker compound, comprising a Polar group selected from the following, or a stereoisomer or salt thereof:

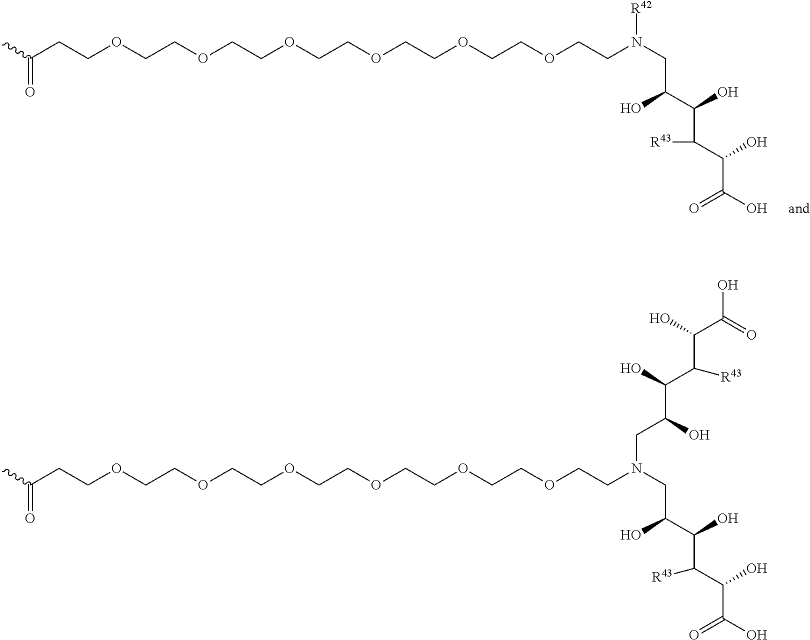

wherein each R⁴² is independently selected from a linear monosaccharide and a substituted linear monosaccharide; each R⁴³ is independently selected from carboxyl, ester, and amide; and the wavy line is an attachment to the Amino Acid unit.

In some embodiments, provided is a Linker compound, comprising a Polar group selected from the following, or a stereoisomer or salt thereof:

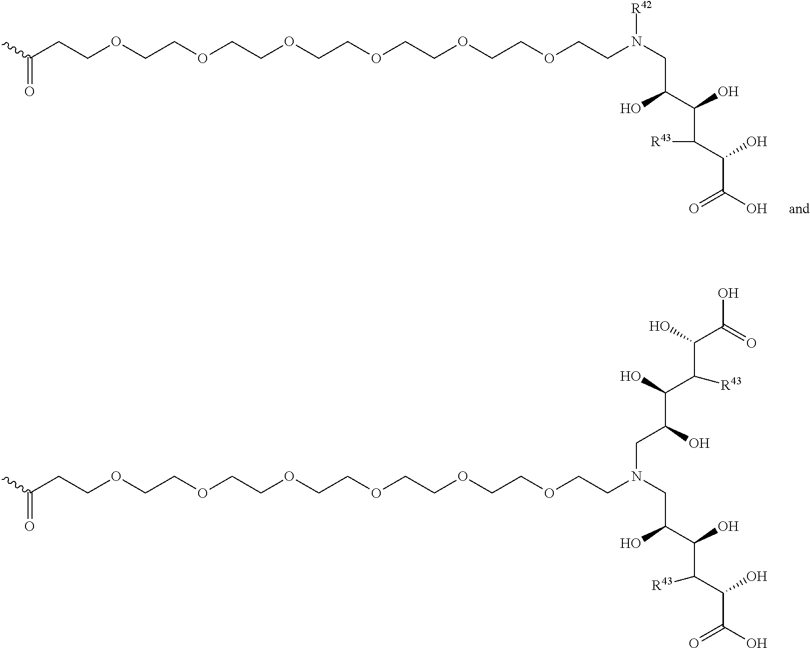

wherein each R⁴² is independently selected from a linear monosaccharide and a substituted linear monosaccharide; each R⁴³ is independently selected from alkyl, O-alkyl, aryl, O-aryl, carboxyl, ester, and amide; and the wavy line is an attachment to the Amino Acid unit.

In some embodiments, provided is a Linker compound, wherein one of R⁴ and R⁵ is a —C(O)— polyhydroxyl group or substituted —C(O)-polyhydroxyl group, and the other of R⁴ and R⁵ is a H, —C(O)— polyhydroxyl group, substituted —C(O)-polyhydroxyl group, polyhydroxyl group or substituted polyhydroxyl group; wherein the substituted —C(O)-polyhydroxyl group and polyhydroxyl group are substituted with a monosaccharide, a disaccharide, a polysaccharide, carboxyl, ester, or amide.

In some embodiments, provided is a Linker compound, wherein one of R⁴ and R⁵ is a —C(O)— polyhydroxyl group or substituted —C(O)-polyhydroxyl group, and the other of R⁴ and R⁵ is a H, —C(O)— polyhydroxyl group, substituted —C(O)-polyhydroxyl group, polyhydroxyl group or substituted polyhydroxyl group; wherein the substituted —C(O)-polyhydroxyl group and polyhydroxyl group are substituted with a monosaccharide, a disaccharide, a polysaccharide, alkyl, —O-alkyl, aryl, carboxyl, ester, or amide.

In some embodiments, provided is a Linker compound, wherein —(NR⁴R⁵) is selected from the following, or a stereoisomer or salt thereof:

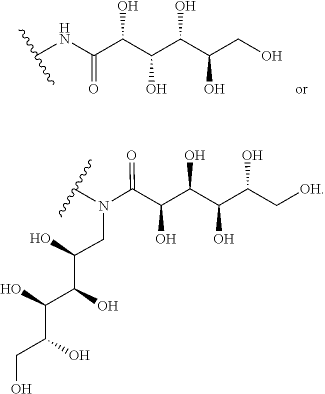 [SMILES: *NC(=O)[C@H](O)[C@@H](O)[C@H](O)[C@H](O)CO ; "or" ; *N(C[C@H](O)[C@@H](O)[C@H](O)[C@H](O)CO)C(=O)[C@H](O)[C@@H](O)[C@H](O)[C@H](O)CO]

In some embodiments, provided is a Linker compound, comprising a Polar group selected from the following, or a stereoisomer or salt thereof:

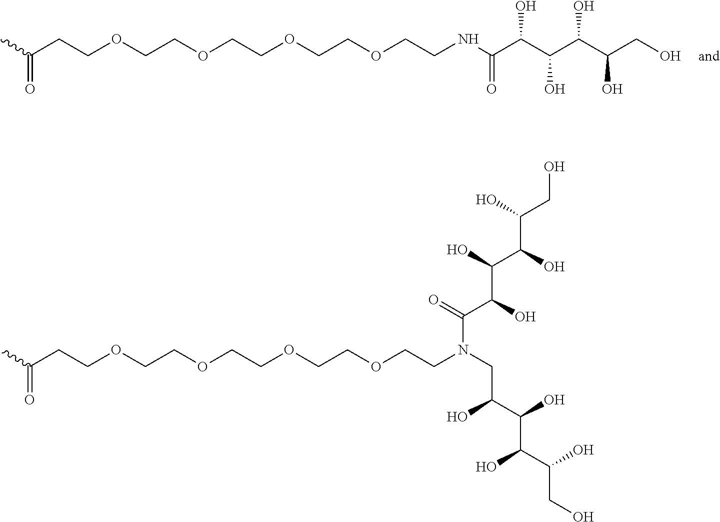

wherein the wavy line is an attachment to the Amino Acid unit.

In some embodiments, provided is a Linker compound, wherein R⁴ and R⁵ are independently selected from a H, substituted —C₁-C₈ alkyl, substituted —C₁-C₄ alkyl or substituted —C₁-C₃ alkyl; and wherein at least one of R⁴ and R⁵ is not H; wherein substituted —C₁-C₈ alkyl, —C₁-C₄ alkyl and —C₁-C₃ alkyl are substituted with hydroxyl and/or carboxyl.

In some embodiments, provided is a Linker compound, wherein —(NR⁴R⁵) is selected from the following, or a stereoisomer or salt thereof:

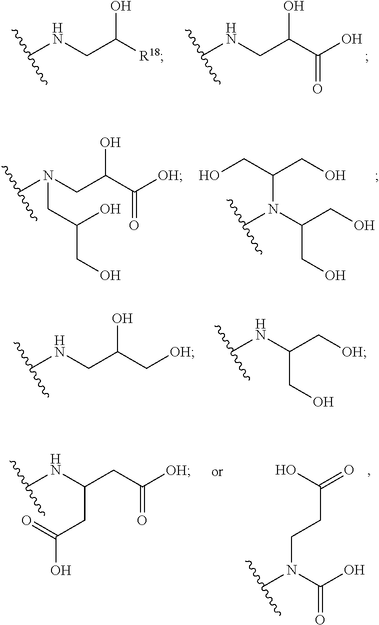

wherein R¹⁸ is selected from OH, CH₂OH, COOH or —C₁-C₆ alkyl substituted with hydroxyl or carboxyl.

In some embodiments, provided is a Linker compound, wherein —(NR⁴R⁵) is selected from the following, or a stereoisomer or salt thereof:

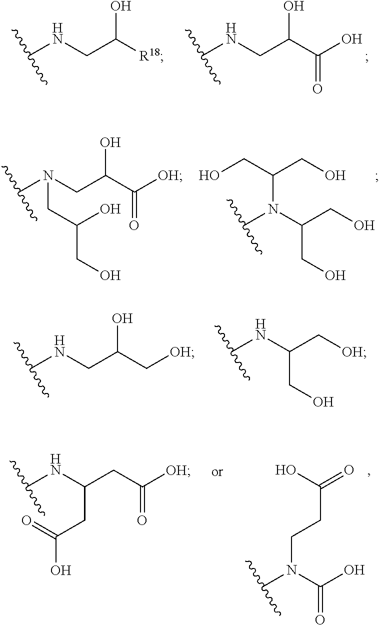

wherein R⁸ is selected from H, OH, CH₂OH, COOH or —C₁-C₆ alkyl substituted with hydroxyl or carboxyl.

In some embodiments, provided is a Linker compound, comprising a Polar group selected from the following, or a stereoisomer or salt thereof:

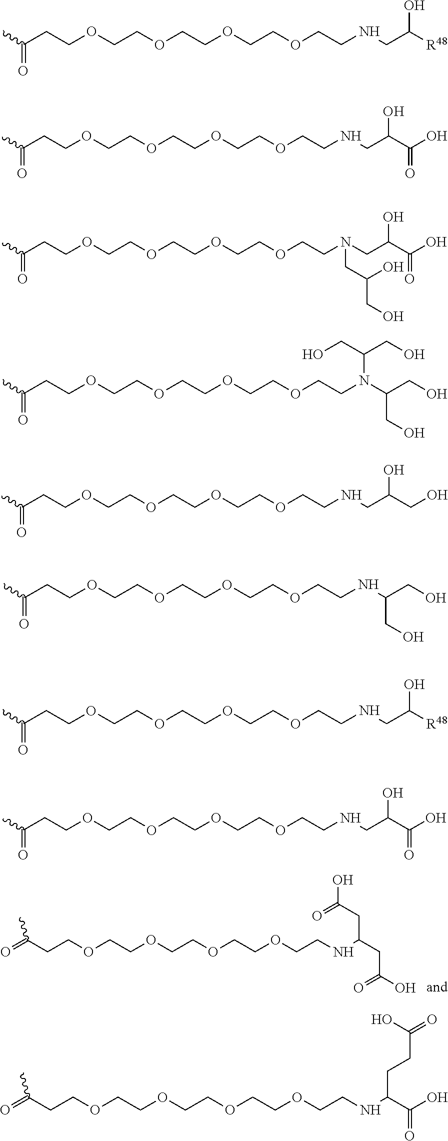

wherein R⁴⁸ is selected from OH, CH₂OH, COOH or —C₁-C₆ alkyl substituted with hydroxyl or carboxyl; and the wavy line is an attachment to the Amino Acid unit.

In some embodiments, provided is a Linker compound, comprising a Polar group selected from the following, or a stereoisomer or salt thereof:

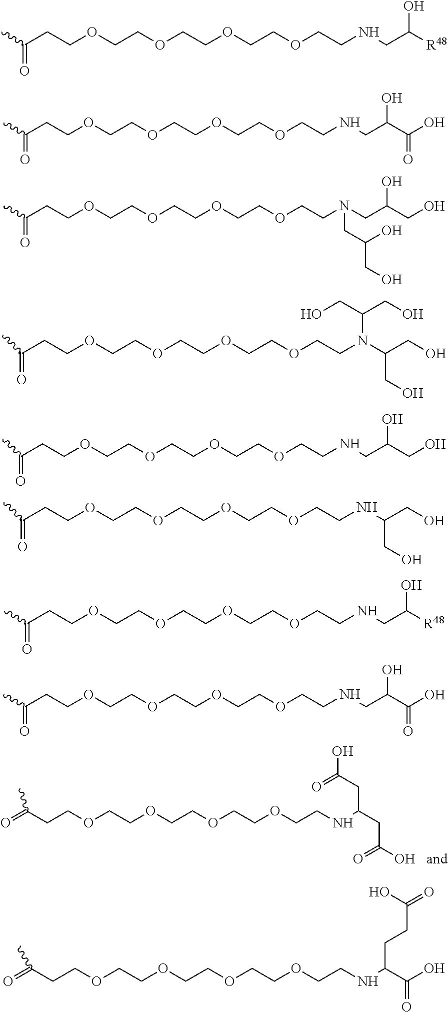

wherein R⁴⁸ is selected from H, OH, CH₂OH, COOH or —C₁-C₆ alkyl substituted with hydroxyl or carboxyl; and the wavy line is an attachment to the Amino Acid unit.

In some embodiments, provided is a Linker compound, wherein one of R⁴ and R⁵ is selected from H, substituted —C(O)—C₁-C₈ alkyl, substituted —C(O)—C₁-C₄ alkyl, and substituted —C(O)—C₁-C₃ alkyl and the other of R⁴ and R⁵ is selected from substituted —C(O)—C₁-C₈ alkyl, substituted —C(O)—C₁-C₄ alkyl, substituted —C(O)—C₁-C₃ alkyl, substituted —C₁-C₈ alkyl, substituted —C₁-C₄ alkyl, and substituted —C₁-C₃ alkyl, wherein substituted —C(O)—C₁-C₈ alkyl, substituted —C(O)—C₁-C₄ alkyl, substituted —C(O)—C₁-C₃ alkyl, substituted —C₁-C₈ alkyl, —C₁-C₄ alkyl and —C₁-C₃ alkyl are substituted with hydroxyl and/or carboxyl.

In some embodiments, provided is a Linker compound, wherein —(NR⁴R⁵) is selected from the following, or a stereoisomer or salt thereof:

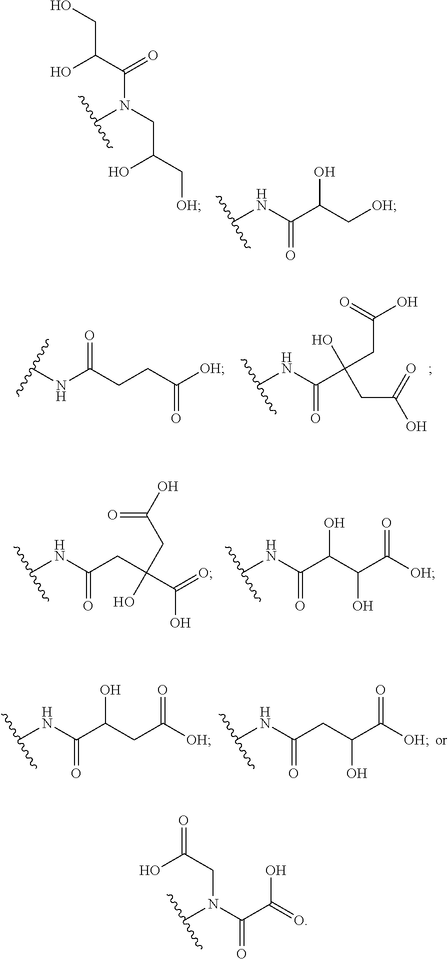

In some embodiments, provided is a Linker compound, comprising a Polar group selected from the following, or a stereoisomer or salt thereof:

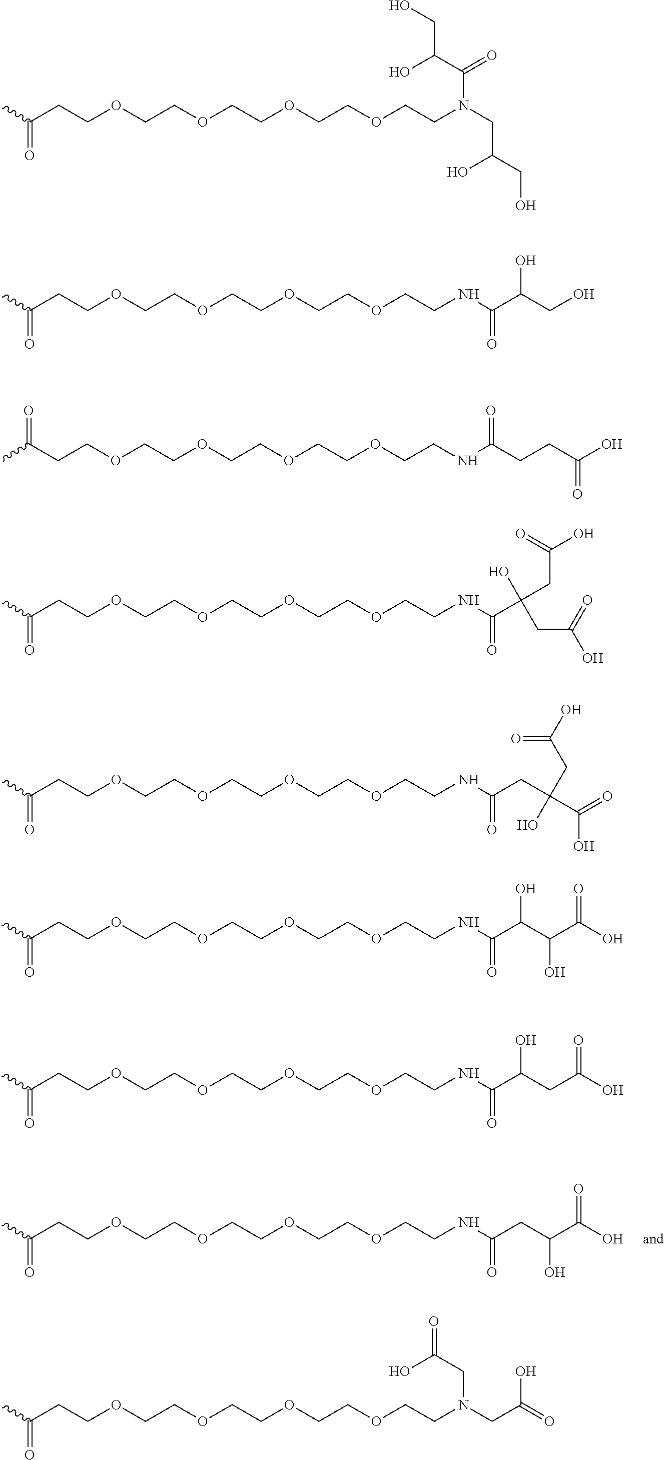

wherein the wavy line is an attachment to the Amino Acid unit.

In some embodiments, provided is a Linker intermediate or Linker, wherein R²⁴ and R²⁵ of the Polymer unit are selected from H and optionally substituted aryl; provided that both R²⁴ and R²⁵ are not H, wherein the optional substituents are as defined herein, for example in some embodiments the optional substitutent is halo, such as F, Cl, or Br. In some embodiments, provided is a Linker intermediate or Linker wherein the Polymer unit is selected from the following, or a salt thereof:

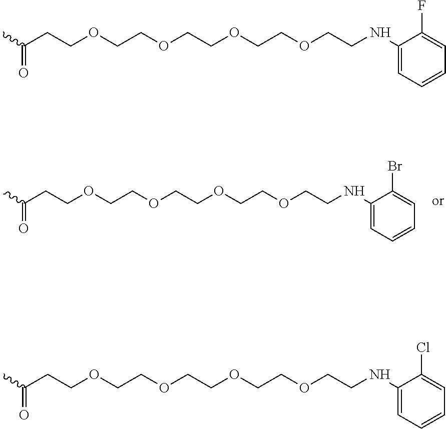

wherein the wavy line at the left side indicates the attachment site to the subunit of the Amino Acid unit or the portion of the Linker subunit.

In some embodiments, provided is a Linker compound, wherein R⁴ and R⁵ together form an optionally substituted C₃-C₈ heterocycle or heteroaryl.

In some embodiments, provided is a Linker compound, wherein the Polymer unit is:

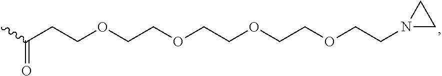

or a salt thereof.

In some embodiments, provided is a Linker compound, wherein R⁴ and R⁵ are independently selected from H and a chelator, wherein the chelator is optionally attached to the nitrogen of —NR⁴R⁵ by an alkylene, arylene, carbocyclyl, heteroarylene or heterocarbocyclyl; provided that both R⁴ and R⁵ are not H.

In some embodiments, provided is a Linker compound, wherein the chelator is selected from ethylenediaminetetraacetic acid (EDTA), diethylenetriaminepentaacetic acid (DTPA), triethylenetetraminehexaacetic acid (TTHA), benzyl-DTPA, 1,4,7,10-tetraazacyclododecane-N,N′,N″,N′″-tetraacetic acid (DOTA), benzyl-DOTA, 1,4,7-triazacyclononane-N,N′,N″-triacetic acid (NOTA), benzyl-NOTA, 1,4,8,11-tetraazacyclotetradecane-1,4,8,11-tetraacetic acid (TETA) and N,N′-dialkyl substituted piperazine.

In some embodiments, provided is a Linker compound, comprising a Polar group selected from the following:

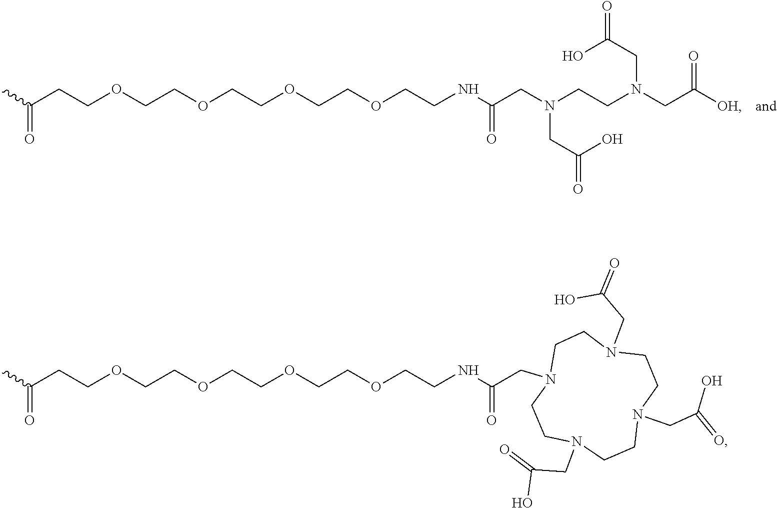

or a stereoisomer or salt thereof, wherein the wavy line is an attachment to the Amino Acid unit.

In some embodiments, provided is a Linker compound, wherein R⁴ and R⁵ are independently selected from a H, a polyhydroxyl-ether group, a substituted polyhydroxyl-ether group.

In some embodiments, provided is a Linker compound, wherein —(NR⁴R⁵) is selected from the following, or a stereoisomer or salt thereof:

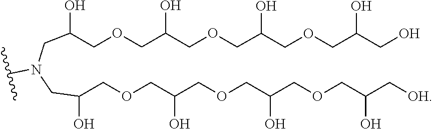 [SMILES: *N(CC(O)COCC(O)COCC(O)COCC(O)CO)CC(O)COCC(O)COCC(O)COCC(O)CO]

In some embodiments, provided is a Linker compound, comprising a Polar group having a formula selected from the following:

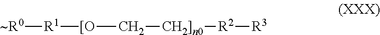

or a stereoisomer or salt thereof, wherein:
   R⁰ is a functional group for attachment to a subunit of the Amino Acid unit;
   R¹ and R² are each independently, a bond or C₁-C₃ alkylene groups;
   R³ is selected from an optionally substituted C₃-C₁₀ carbocycle, thiourea, optionally substituted thiourea, urea, optionally substituted urea, sulfamide, alkyl sulfamide, acyl sulfamide, optionally substituted alkyl sulfamide, optionally substituted acyl sulfamide, sulfonamide, optionally substituted sulfonamide, guanidine, including alkyl and aryl guanidine, phosphoramide, or optionally substituted phosphoramide; or R³ is selected from azido, alkynyl, substituted alkynyl, —NH—C(O)-alkynyl, —NH—C(O)-alkynyl-R⁵, cyclooctyne; —NH-cyclooctyne, —NH—C(O)-cyclooctyne, or —NH-(cyclooctyne)₂; wherein R⁵ is selected from optionally substituted alkyl, optionally substituted alkenyl, optionally substituted alkynyl, optionally substituted carbocycle, optionally substituted aryl, optionally substituted heterocarbocycle, or optionally substituted heteroaryl; and
   n0 is 2 to 26;

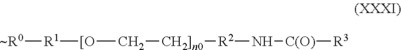

or a stereoisomer or salt thereof, wherein:
   R⁰ is a functional group for attachment to a subunit of the Amino Acid unit;
   R¹ and R² are each, independently, a bond or C₁-C₃ alkylene groups;
   R³ is a branched polyethylene glycol chain, each branch having 1 to 26 ethylene glycol subunits and each branch having an R⁴ at its terminus;
   R⁴ is azido, alkynyl, alkynyl-R⁵, cyclooctyne or cyclooctyne-R⁵, wherein R⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
   n0 is 2 to 26;

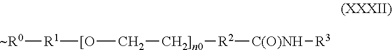

or a stereoisomer or salt thereof, wherein:
   R⁰ is a functional group for attachment to a subunit of the Amino Acid unit;
   R¹ and R² are each, independently, a bond or C₁-C₃ alkylene groups;
   R³ is a branched polyethylene glycol chain, each branch, independently, having 1 to 26 ethylene glycol subunits and each branch having an R⁴ at its terminus;
   R⁴ is azido, alkynyl, alkynyl-R⁵, cyclooctyne or cyclooctyne-R⁵, wherein R⁵ is selected from optionally substituted alkyl, optionally substituted alkenyl, optionally substituted alkynyl, optionally substituted carbocycle, optionally substituted aryl, optionally substituted heterocarbocycle and optionally substituted heteroaryl; and
   n0 is 2 to 26;

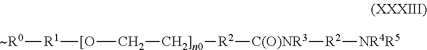

or a stereoisomer or salt thereof, wherein:
   R⁰ is a functional group for attachment to a subunit of the Amino Acid unit;
   R³ is H or R²—NR⁴R⁵;
   R¹ and R² are each, independently, a bond or C₁-C₃ alkylene groups;
   R⁴ and R⁵ are each independently selected from a H, polyhydroxyl group, substituted polyhydroxyl group, —C(O)-polyhydroxyl group, substituted —C(O)-polyhydroxyl group, wherein R⁴ and R⁵ are not both H; and
   n0 is 2 to 26;

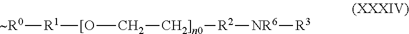

or a stereoisomer or salt thereof, wherein:
   R⁰ is a functional group for attachment to a subunit of the Amino Acid unit;
   R¹ and R² are each, independently, a bond or C₁-C₃ alkylene groups;
   R³ is a branched polyethylene glycol chain, each branch having 1 to 26 ethylene glycol subunits and each branch having an R⁴ at its terminus;
   R⁶ is C₁-C₃ alkylene, C₁-C₃ alkylene-C(O), —C(O)—C₁-C₃ alkylene, or —C(O)—C₁-C₃ alkylene-C(O);
   R⁴ is azido, alkynyl, alkynyl-R⁵, cyclooctyne or cyclooctyne-R⁵, wherein R⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
   n0 is 2 to 26;

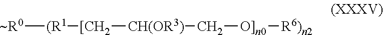

or a stereoisomer or salt thereof, wherein:
   R⁰ is a functional group for attachment to a subunit of the Amino Acid unit;
   each R¹ is independently a bond, —O— or C₁-C₃ alkylene group;
   each R³ is independently H, —[CH₂—CH(OH)—CH₂—O]ₙ₀—R⁶, —C(O)—NR⁴R⁵ or —C(O)N(R^{N})—C₁-C₆alkylene-NR⁴R⁵;
   R^{N} is H or C₁-C₄alkyl;
   R⁴ and R⁵ are each independently selected from a H, polyhydroxyl group, or substituted polyhydroxyl group, wherein R⁴ and R⁵ are not both H;
   each R⁶ is independently H, C₁-C₆alkylene-C(OH)H—NR⁷R⁸, C₁-C₆alkylene-C(OH)H—C₁-C₆alkylene-NR⁷R⁸, —C(O)—NR⁴R⁵, —C(O)N(R^{N})—C₁-C₆alkylene-NR⁴R⁵, C₁-C₆alkylene-C(O)NR⁴R⁵ or C₁-C₆alkylene-CO₂R⁹;
   each R⁹ is independently H or C₁-C₆ alkyl;
   R⁷ and R⁸ are each independently selected from a H, polyhydroxyl group, substituted polyhydroxyl group, —C(O)-polyhydroxyl group, substituted —C(O)-polyhydroxyl group;
   each n0 is independently 1 to 26; and
   n2 is 1 or 2;

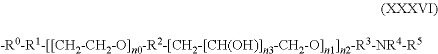

or a stereoisomer or salt thereof, wherein:
   R⁰ is a functional group for attachment to a subunit of the Amino Acid unit;
   R¹, R² and R³ are each independently a bond or C₁-C₃ alkylene group;
   R⁴ and R⁵ are each independently selected from a H, polyhydroxyl group, substituted polyhydroxyl group, —C(O)-polyhydroxyl group, substituted —C(O)-polyhydroxyl group, wherein R⁴ and R⁵ are not both H;
   each n0 is independently 0 to 26, and each n1 is independently 0 to 26, with the proviso that at least one of n0 or n1 is 2 to 26;
   n2 is Ito 5;
   each n3 is independently 1 or 2;

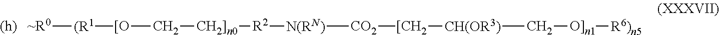

or a stereoisomer or salt thereof, wherein:
   R⁰ is a functional group for attachment to a subunit of the Amino Acid unit;
   R¹ and R² are each, independently, a bond or C₁-C₃ alkylene groups;
   R^{N} is H or C₁-C₄alkyl;
   R⁴ and R⁵ are each independently selected from a H, polyhydroxyl group, or substituted polyhydroxyl group, wherein R⁴ and R⁵ are not both H;
   each R³ is independently H, —[CH₂—CH(OH)—CH₂—O]ₙ₀—R⁶ or —C(O)N(R^{N})—C₁-C₆alkylene-NR⁴R⁵;
   each R⁶ is independently H, C₁-C₆alkylene-C(OH)H—NR⁷R⁸, C₁-C₆alkylene-C(OH)H—C₁-C₆alkylene-NR⁷R⁸, —C(O)N(R^{N})—C₁-C₆alkylene-NR⁴R⁵, C₁-C₆alkylene-C(O)NR⁴R⁵ or C₁-C₆alkylene-CO₂R⁹;
   each R⁹ is independently H or C₁-C₆ alkyl;
   R⁷ and R⁸ are each independently selected from a H, polyhydroxyl group, substituted polyhydroxyl group, —C(O)-polyhydroxyl group, substituted —C(O)-polyhydroxyl group;
   n0 is 2 to 26;
   n1 is 1 to 26; and
   n5 is 1 or 2;

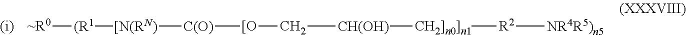

or a stereoisomer or salt thereof, wherein:
   R⁰ is a functional group for attachment to a subunit of the Amino Acid unit;
   R¹ and R² are each, independently, a bond or C₁-C₃ alkylene groups;
   R^{N} is H or C₁-C₄alkyl;
   R⁴ and R⁵ are each independently selected from a H, polyhydroxyl group, or substituted polyhydroxyl group, wherein R⁴ and R⁵ are not both H;
   n0 is 2 to 26;
   n1 is 2 to 4; and
   n5 is 1, 2 or 3;

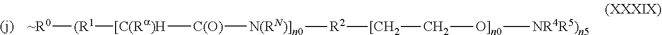

or a stereoisomer or salt thereof, wherein:
   R⁰ is a functional group for attachment to a subunit of the Amino Acid unit;
   R¹ and R² are each, independently, a bond, C₁-C₃ alkylene, —C₁-C₃alkylene-[O—CH₂—CH₂—]ₙ₀, —[CH₂—CH₂—O]ₙ₀—C₁-C₃alkylene-, or —C₁-C₃alkylene-[O—CH₂—CH₂—]ₙ₀—C(O)—;
   each R^{α} is independently H or —R²—NR⁴R⁵;
   each R^{N} is independently H, C₁-C₆ alkyl or —R²—NR⁴R⁵;
   R⁴ and R⁵ are each independently selected from a H, polyhydroxyl group, substituted polyhydroxyl group, —C(O)-polyhydroxyl group, substituted —C(O)-polyhydroxyl group, a chelator, and —C(O)—R, wherein R is a Sugar unit of formula (XII) or (XIII); or —NR⁴R⁵ together from a C₃-C₈ heterocycle, wherein R⁴ and R⁵ are not both H;
   each n0 is independently 0 to 26, with the proviso that at least one n0 is 2 to 26; and
   n5 is 1 or 2; or

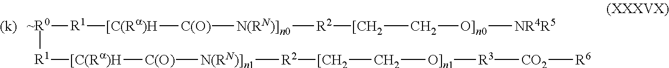

or a stereoisomer or salt thereof, wherein:
   R⁰ is a functional group for attachment to a subunit of the Amino Acid unit;
   R¹, R² and R³ are each, independently, a bond, C₁-C₃ alkylene, —C₁-C₃alkylene-[O—CH₂—CH₂—]ₙ₀, —[CH₂—CH₂—O]ₙ₀—C₁-C₃alkylene- or —C₁-C₃alkylene-[O—CH₂—CH₂—]ₙ₀—C(O)—;
   each R^{α} is independently H or —R²—NR⁴R⁵;
   each R^{N} is independently H, C₁-C₆ alkyl or —R²—NR⁴R⁵;
   R⁴ and R⁵ are each independently selected from a H, polyhydroxyl group, substituted polyhydroxyl group, —C(O)-polyhydroxyl group, substituted —C(O)-polyhydroxyl group, a chelator, —C(O)—R, where R is a Sugar unit of formula (XII) or (XIII); or —NR⁴R⁵ together from a C₃-C₈ heterocycle, wherein R⁴ and R⁵ are not both H;
   R⁶ is H or C₁-C₆ alkyl;
   each n0 is independently 0 to 26, with the proviso that at least one n0 is 2 to 26; and
   each n1 is independently 0 to 26, with the proviso that at least one n1 is 2 to 26.

In some embodiments, provided is a Linker compound, comprising a Polar group having a formula selected from the following, or a stereoisomer or salt thereof:

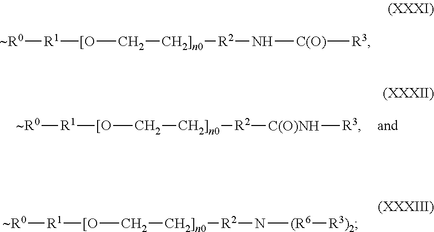

wherein:
   R⁰ is a functional group for attachment to a subunit of the Amino Acid unit;
   R¹ and R² are each, independently, a bond or C₁-C₃ alkylene groups;
   R³ is a branched polyethylene glycol chain, each branch having 1 to 26 ethylene glycol subunits and each branch having an R⁴ at its terminus;
   R⁶ is C₁-C₃ alkylene, —C₁-C₃ alkylene-C(O), —C(O)—C₁-C₃ alkylene or —C(O)—C₁-C₃ alkylene-C(O);
   R⁴ is azido, alkynyl, alkynyl-R⁵, cyclooctyne or cyclooctyne-R⁵, wherein R⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl;
   the wavy line (˜) indicates the attachment site of the Amino Acid unit to R⁰; and
   n0 is 2 to 26.

In some embodiments, provided is a Linker compound, comprising a Polar group formed from a precursor group selected from the following:

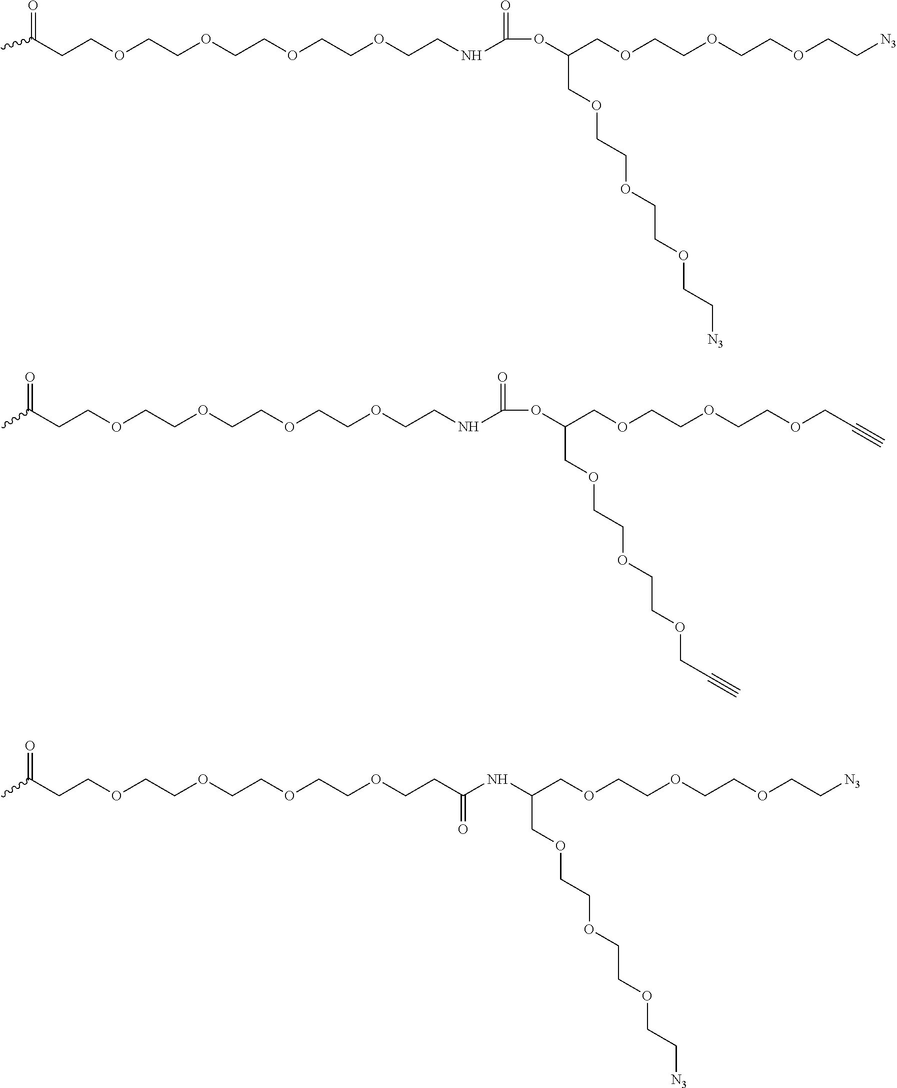

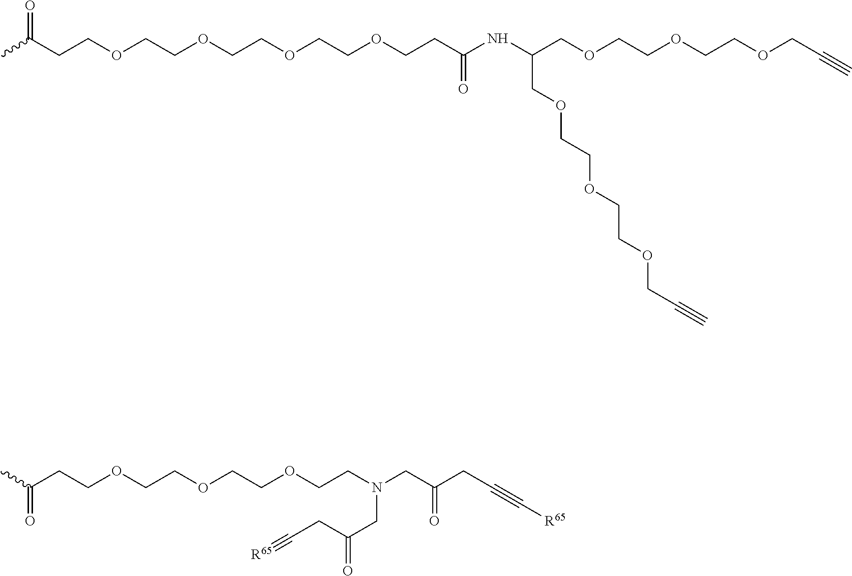

wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and the wavy line is an attachment to the Amino Acid unit.

In some embodiments, provided is a Linker compound, comprising a Polar group having a formula:

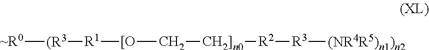

or a stereoisomer or salt thereof, wherein:
   R⁰ is a functional group for attachment to a subunit of the Amino Acid unit;
   R¹ and R² are each, independently, a bond or C₁-C₆ alkylene;
   each R³ is, independently, selected from a bond, C₁-C₁₂ alkylene, —OC₁-C₁₂ alkylene, —C(═O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, or —C(O)NR⁷R⁸, wherein each alkylene is optionally substituted with hydroxyl, SO₃H and/or oxo, R^{a} is H, C₁-C₆ alkyl, a polyhydroxyl group, or a substituted polyhydroxyl group, and one of R⁷ and R⁸ is H or C₁-C₁₂ alkylene and the other is C₁-C₁₂ alkylene, wherein one of the C₁-C₂ alkylenes is bound to NR⁴⁴R⁴⁵ at the nitrogen atom;
   R⁴ and R⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, and wherein R⁴ and R⁵ are not both H;
   n0 is 2 to 26;
   n1 is 1 to 6; and
   n2 is Ito 6.

In some embodiments, provided is a Linker compound, comprising a Polar group having a formula:

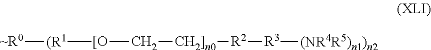

or a stereoisomer or salt thereof, wherein:
   R⁰ is a functional group for attachment to a subunit of the Amino Acid unit;
   R¹ and R² are each, independently, a bond or C₁-C₆ alkylene; each R³ is independently selected from a bond, C₁-C₁₂ alkylene, —OC₁-C₁₂ alkylene, —C(═O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, C(O)—NR^{a}—C₁-C₁₂ alkylene, -heteroarylene, heteroaryl-C₁-C₁₂ alkylene, heteroaryl-C₁-C₁₂ alkylene-C(O)—, or —C(O)NR⁷R⁸, wherein each alkylene is optionally substituted with hydroxyl, SO₃H and/or oxo, R^{a} is H, C₁-C₆ alkyl, a polyhydroxyl group, or a substituted polyhydroxyl group and one of R and R⁸ is H or C₁-C₁₂ alkylene and the other is C₁-C₁₂ alkylene, wherein one of the C₁-C₂ alkylenes is bound to NR⁴⁴R⁴⁵ at the nitrogen atom;
   R⁴ and R⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, and wherein R⁴ and R are not both H;
   n0 is 2 to 26;
   n1 is 1 to 6; and
   n2 is Ito 6.

In some embodiments, provided is a Linker compound, comprising a Polar group having a formula:

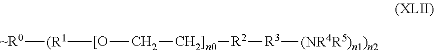

or a stereoisomer or salt thereof, wherein:
   R⁰ is a functional group for attachment to a subunit of the Amino Acid unit;
   R¹ and R² are each, independently, a bond or C₁-C₃ alkylene;
   each R³ is independently selected from a bond, C₁-C₆ alkylene, —OC₁-C₁₂ alkylene, —C(═O)—, —NR^{a}—C₁-C₁₂ alkylene, —C₁-C₆ alkylene-NR^{a}—, —C(O)—C₁-C₆ alkylene, —C₁-C₆ alkylene-C(O)—, —NR^{a}—C₁-C₆ alkylene-C(O)—, —C(O)—C₁-C₆ alkylene-NR^{a}—, —NR^{a}—C(O)—NR^{a}—, —NR^{a}—C(O)—, —NR^{a}—C(O)—C₁-C₆ alkylene, —C(O)—NR^{a}—C₁-C₁₂ alkylene, -heteroarylene, heteroaryl-C₁-C₆ alkylene, heteroaryl-C₁-C₆ alkylene-C(O)—, and —C(O)NR⁷R⁸, wherein each alkylene is optionally substituted with hydroxyl, SO₃H, and/or oxo, R^{a} is H, C₁-C₆ alkyl, a polyhydroxyl group, or a substituted polyhydroxyl group and one of R and R⁸ is H or C₁-C₆ alkylene and the other is C₁-C₁₂ alkylene, wherein one of the C₁-C₂ alkylenes is bound to NR⁴⁴R⁴⁵ at the nitrogen atom;
   R⁴ and R⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, and wherein R⁴ and R⁵ are not both H;
   n0 is 2 to 16;
   n1 is 1 to 4; and
   n2 is Ito 4.

In some embodiments, provided is a Linker compound, wherein R⁰ derives from a functional group of a precursor compound to the Polymer unit, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, or protected forms thereof.

In some embodiments, provided is a Linker compound, wherein R⁰ has one of the following

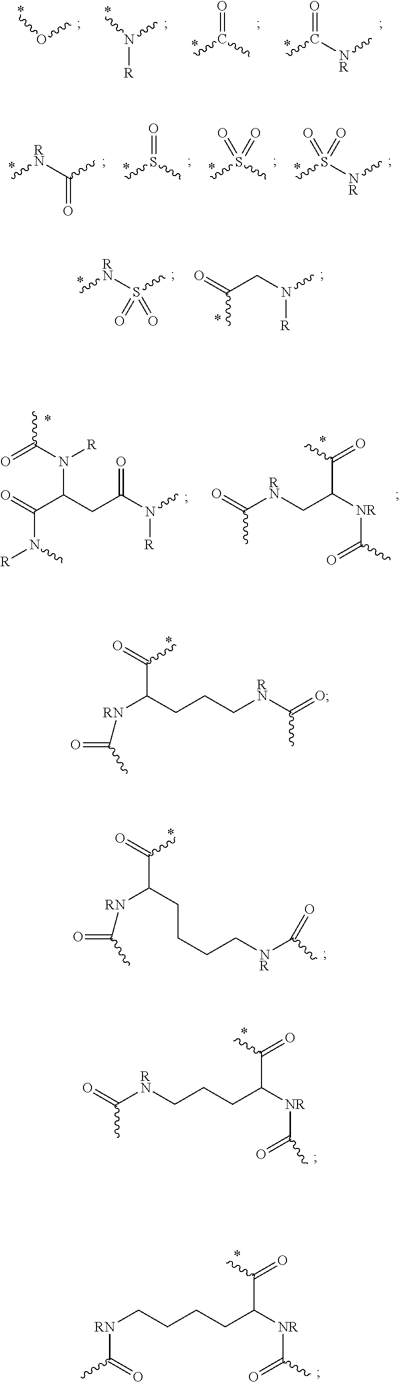

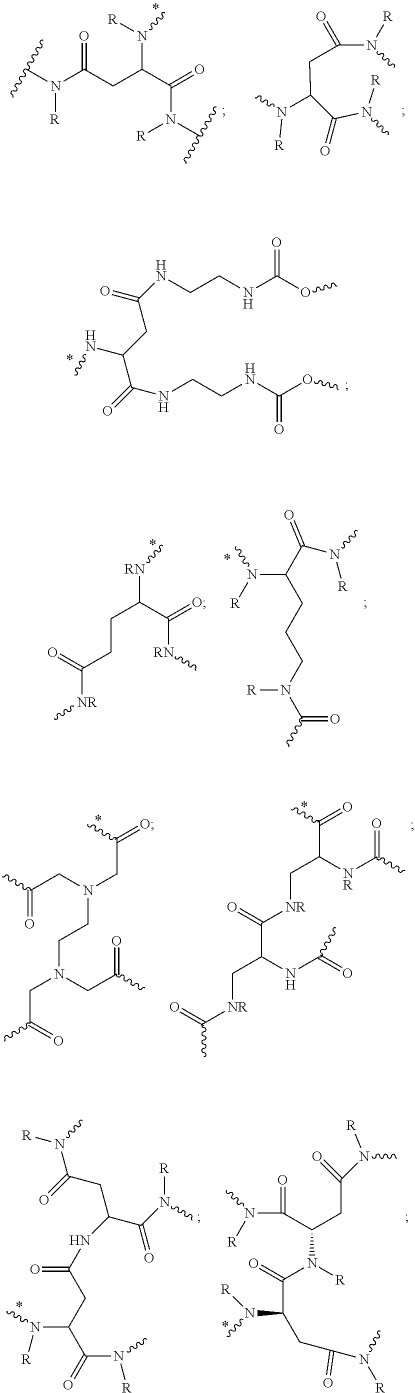

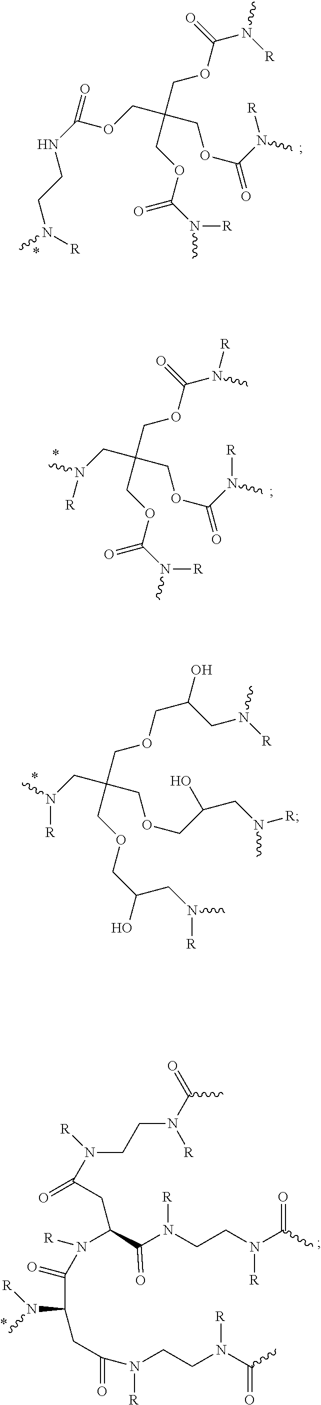

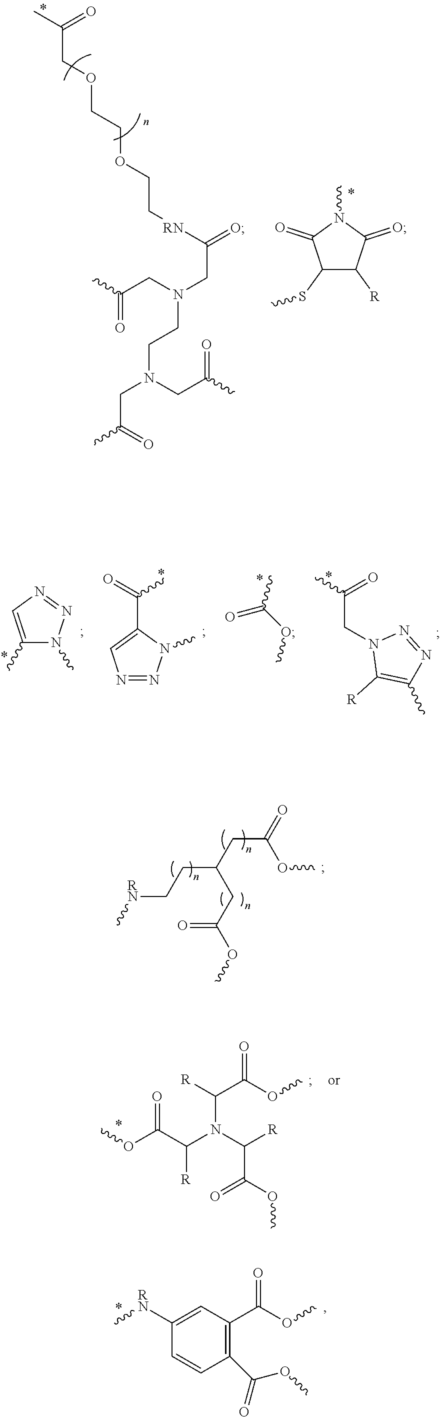

or a stereoisomer thereof, wherein R is H, C₁-C₆ alkyl or polyhydroxyl group, n is 0 to 12, the (*) indicates the attachment site of R⁰ to a subunit of the Amino Acid unit and each ( [symbol] ) indicates the attachment site of R⁰ to the remainder of the Polymer unit.

In some embodiments, provided is a Linker compound, wherein R⁰ has one of the following structures:

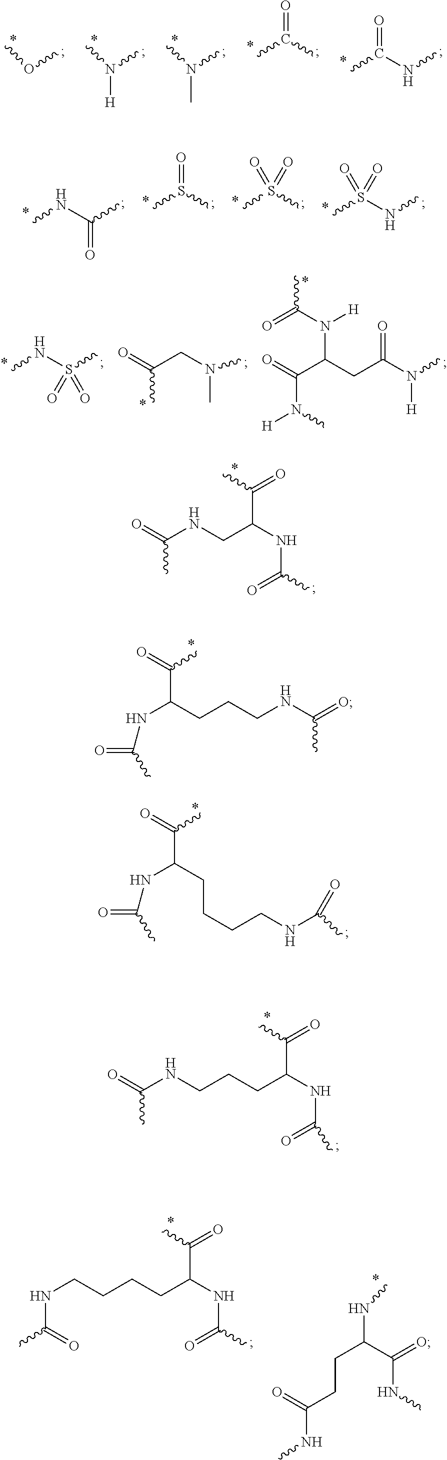

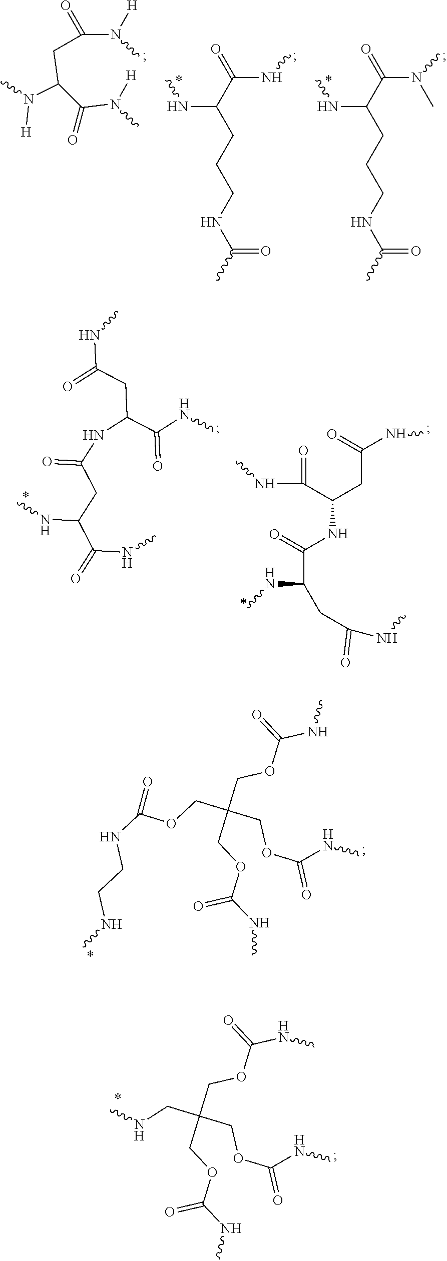

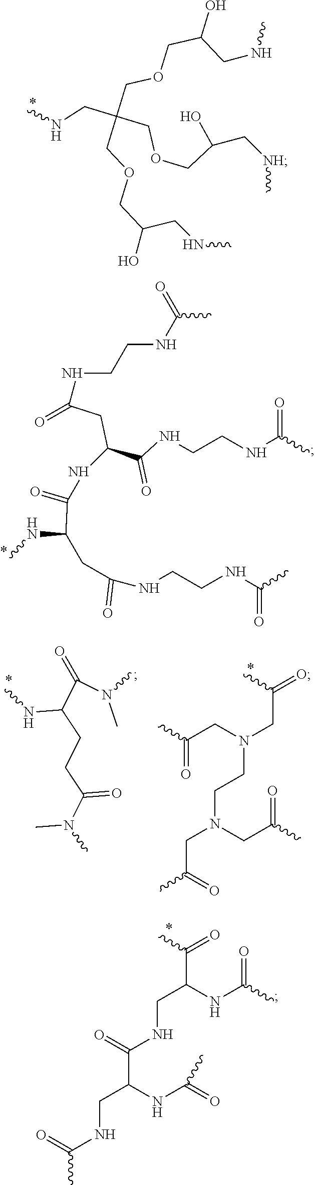

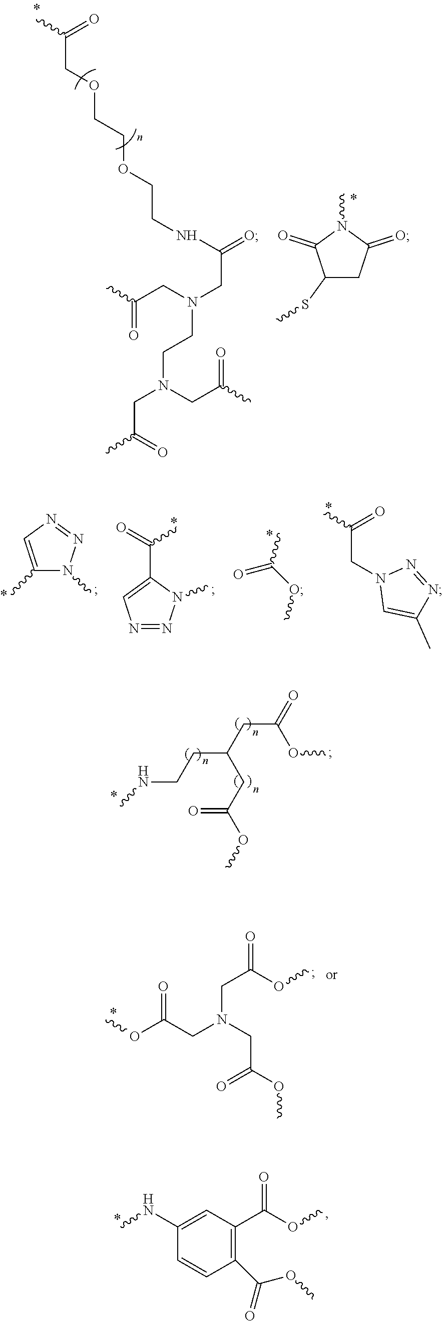

or a stereoisomer thereof, wherein R is H, C₁-C₆ alkyl or polyhydroxyl group, n is 0 to 12, the (*) indicates the attachment site of R⁰ to a subunit of the Amino Acid unit and each ( [symbol] ) indicates an attachment site of R⁰ to the remainder of the Polymer unit.

In some embodiments, provided is a Linker compound, wherein —R³—(NR⁴R⁵)ₙ₁, when R³ is present, has one of the following structures:

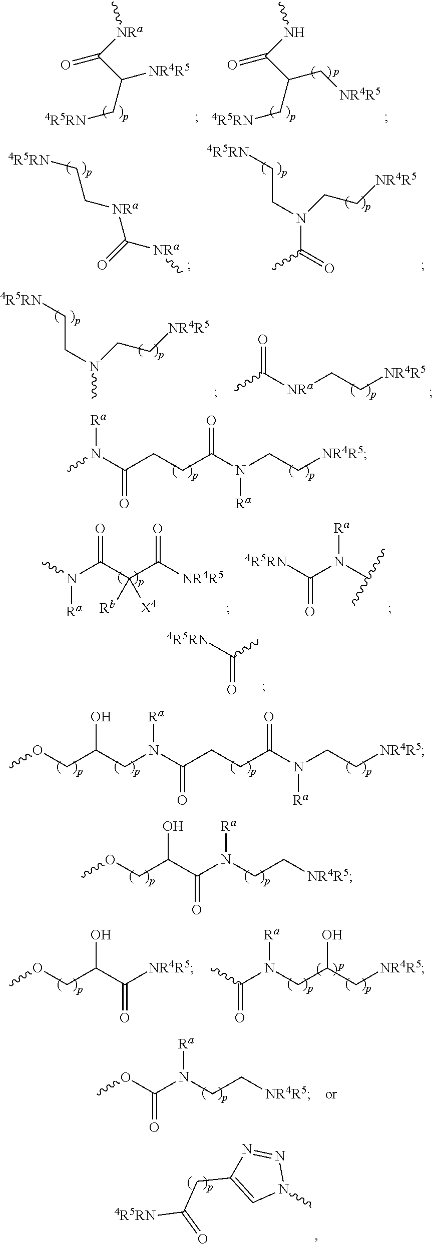

or a stereoisomer thereof, wherein each R^{a} and R^{b} are independently H or C₁₋₆ alkyl, X⁴ is SO₃H, p is 0-8, and the ( [symbol] ) indicates the attachment site of R³ to the remainder of the Polymer unit.

In some embodiments, provided is a Linker compound, wherein —R³—(NR⁴R⁵)ₙ₁, when R³ is present, has one of the following structures:

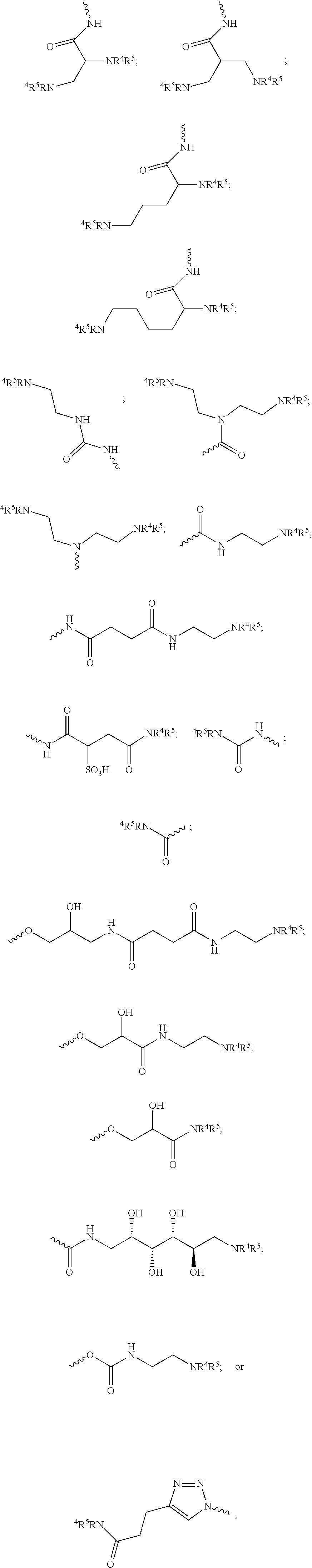

or a stereoisomer thereof, wherein the ( [symbol] ) indicates the attachment site of R³ to the remainder of the Polymer unit.

In some embodiments, provided is a Linker compound, wherein at least one —NR⁴R⁵, when present, has one of the following structures:

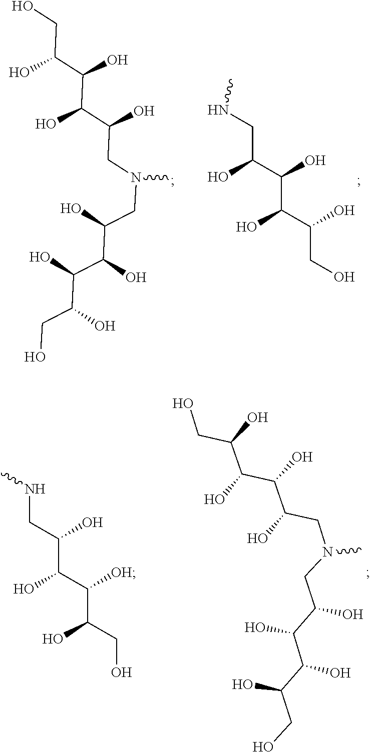

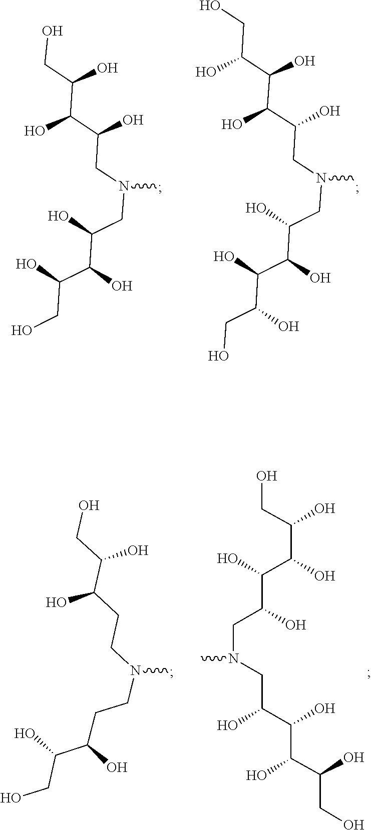

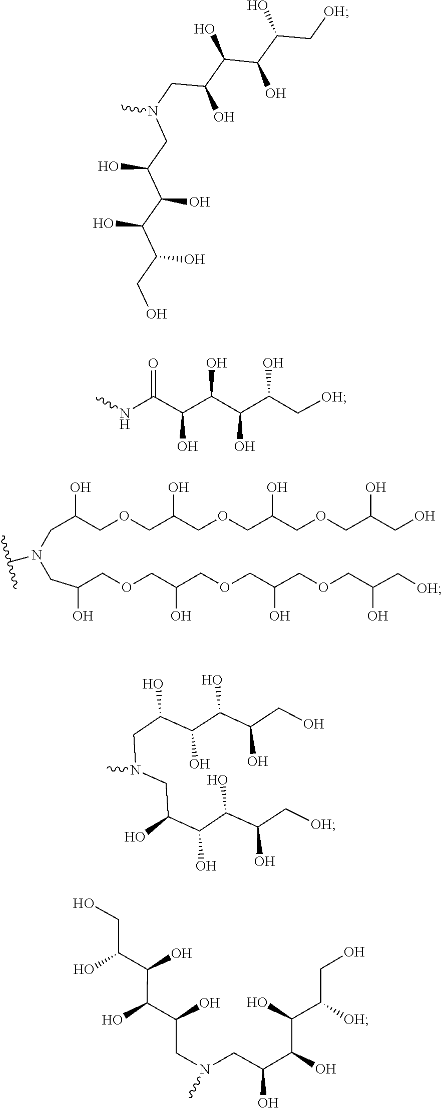

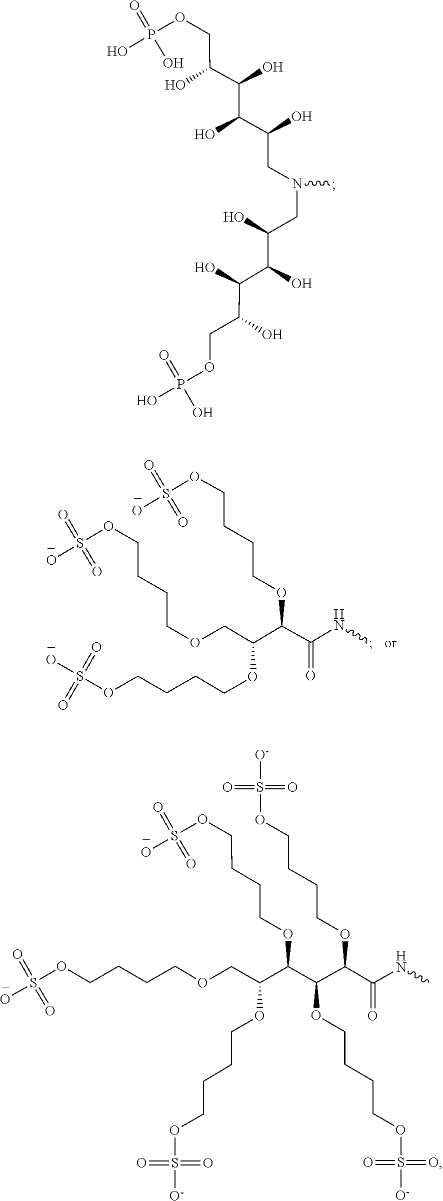

or a stereoisomer thereof, wherein the ( [symbol] ) indicates the attachment site of —NR⁴R⁵ to the remainder of the Polymer unit.

In some embodiments, provided is a Linker compound, comprising a Polar group having one of the following structures prior to attachment to the Linker unit:

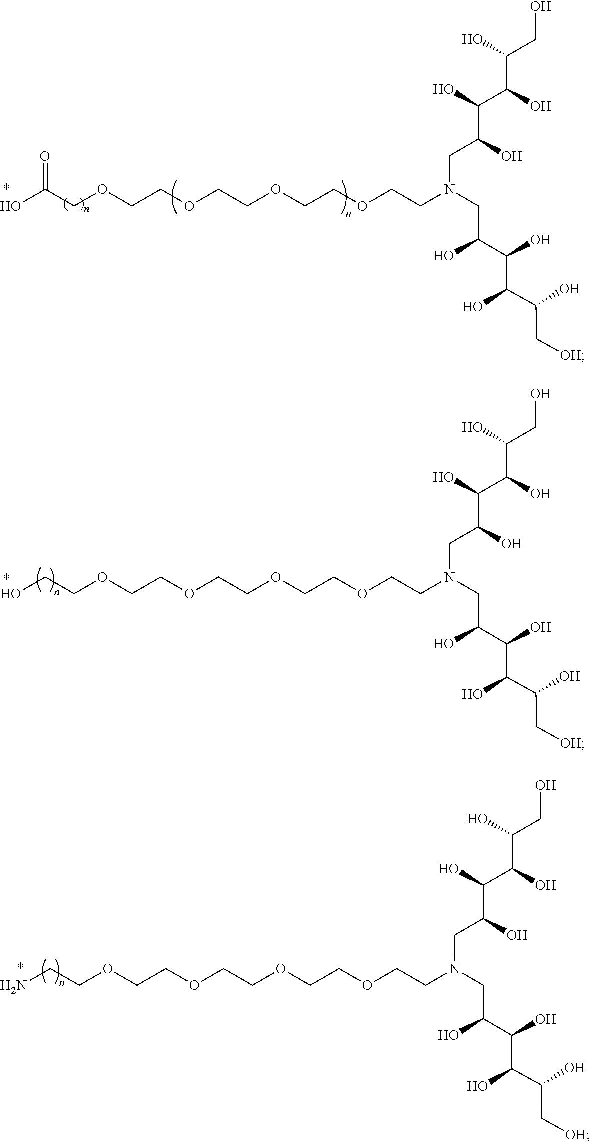

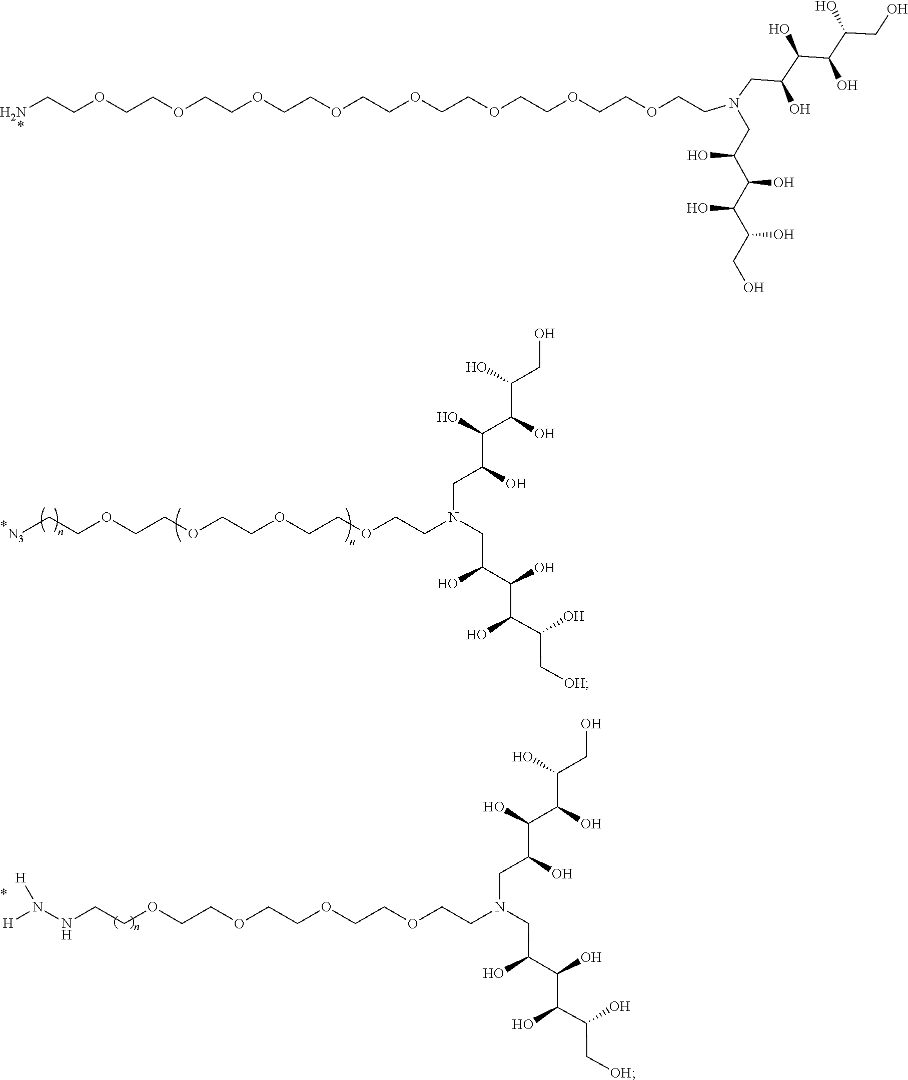

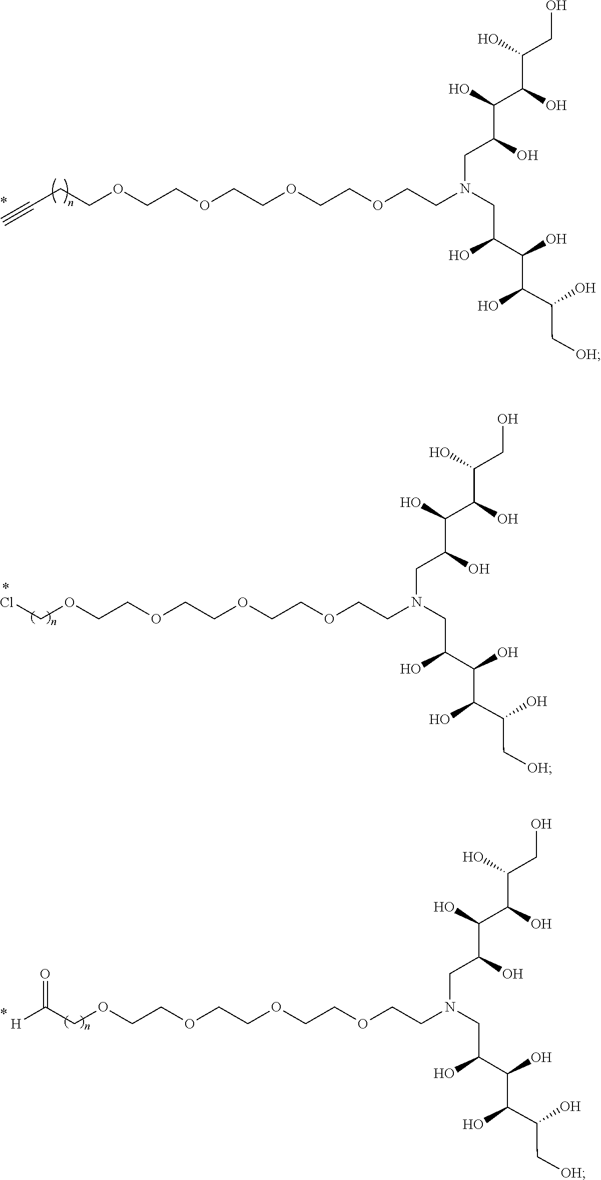

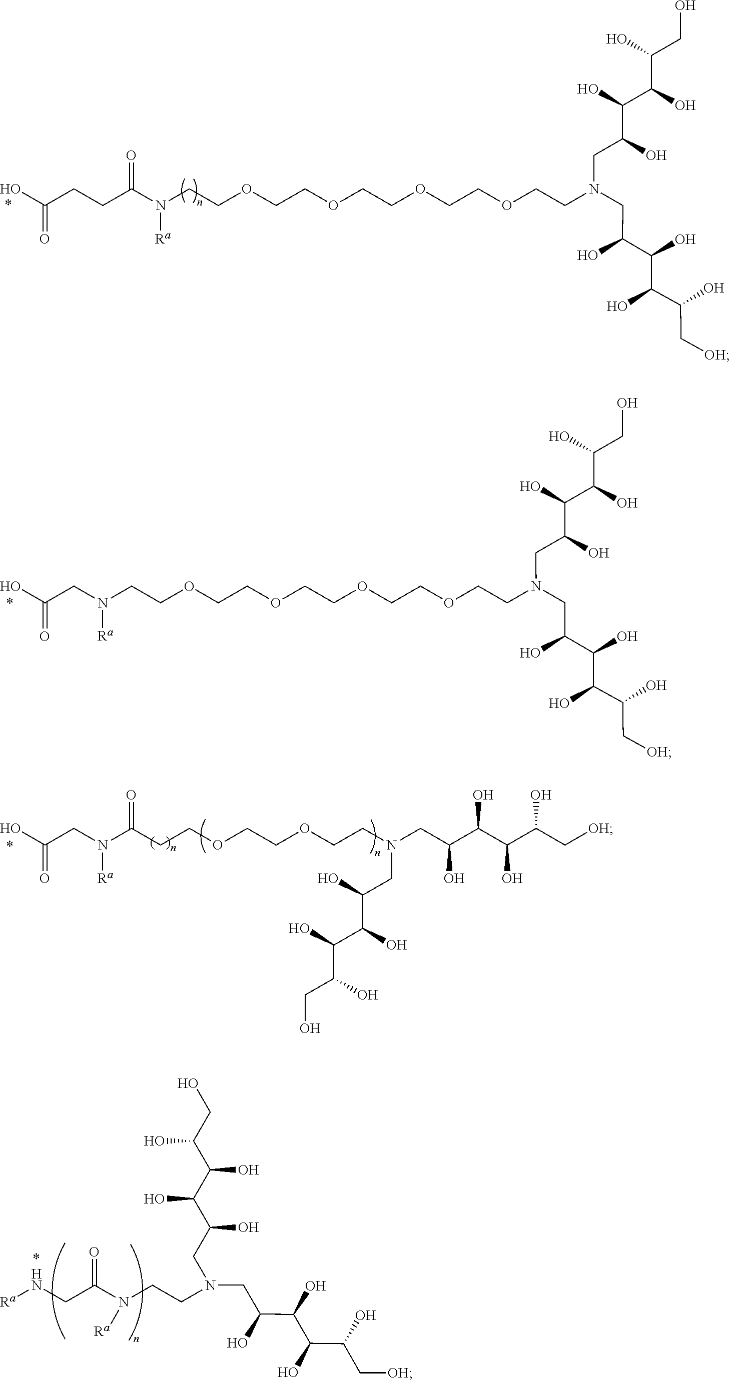

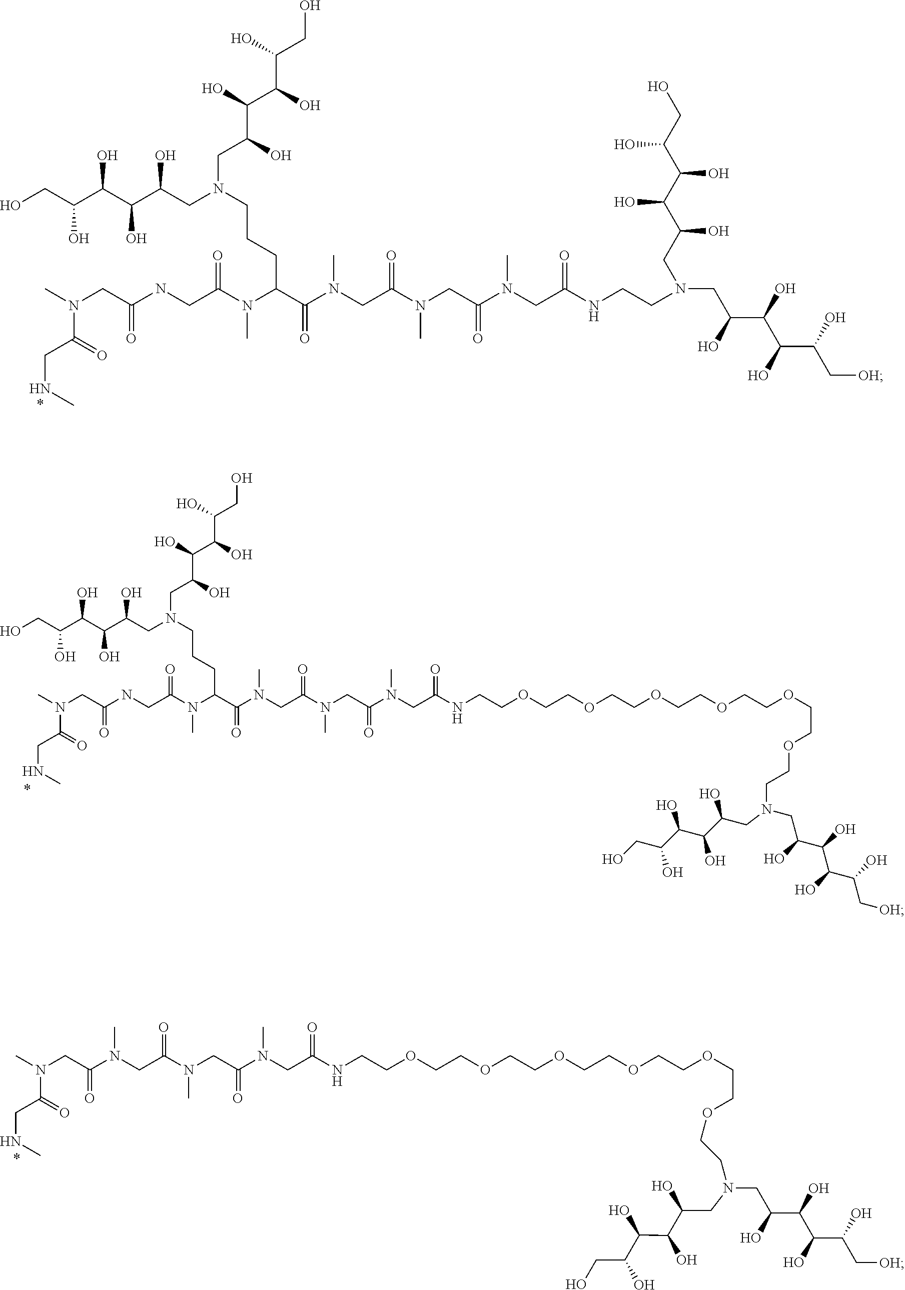

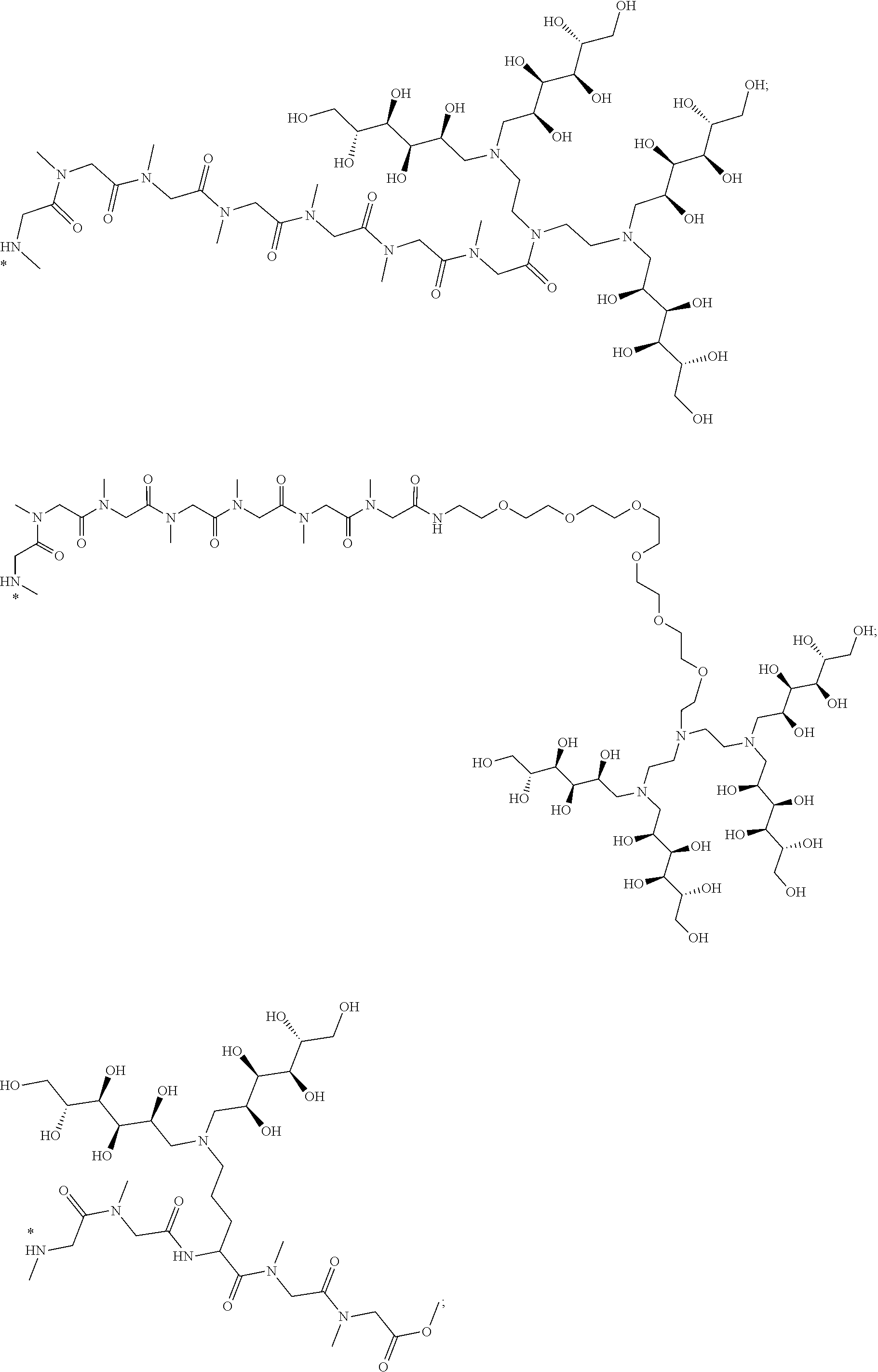

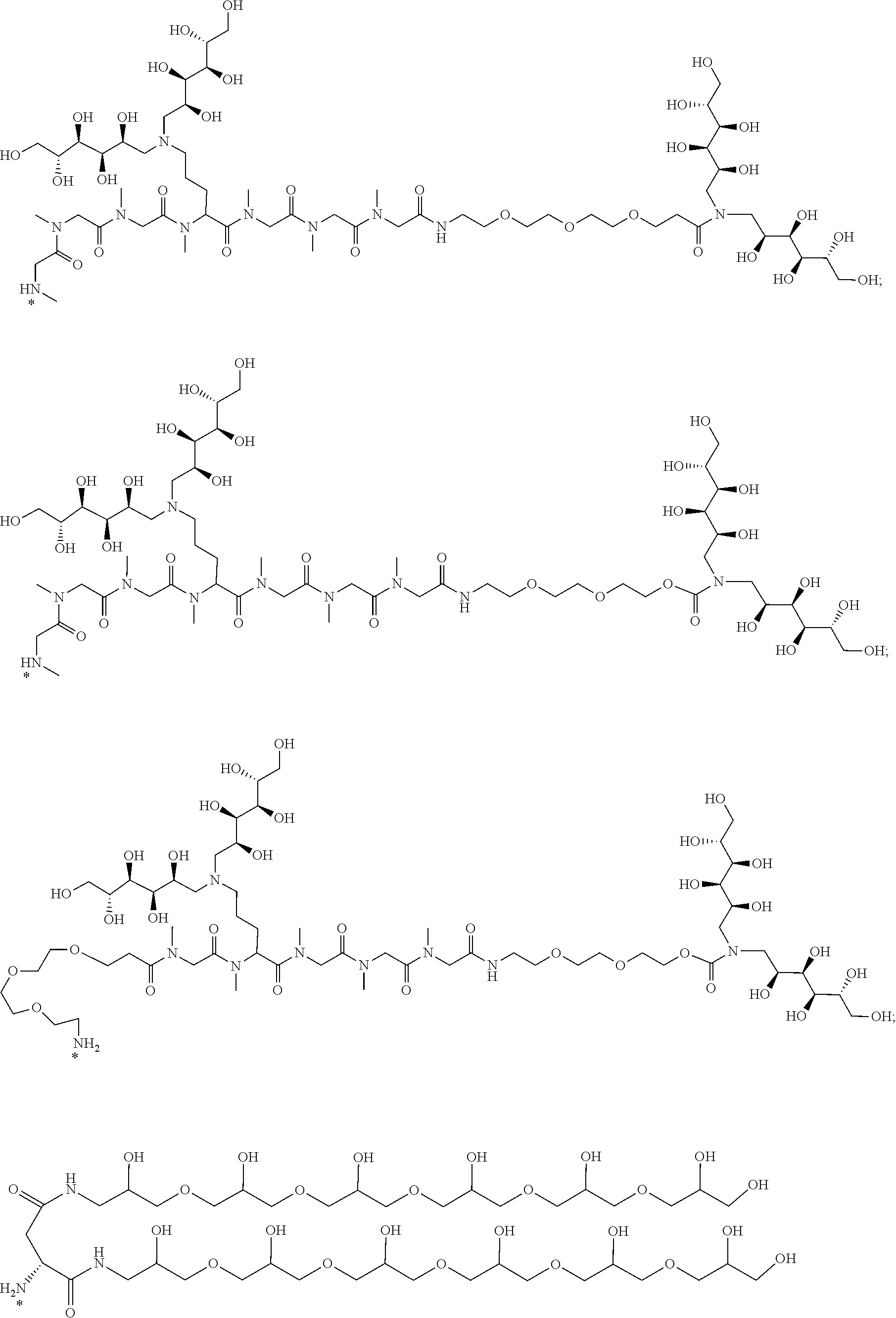

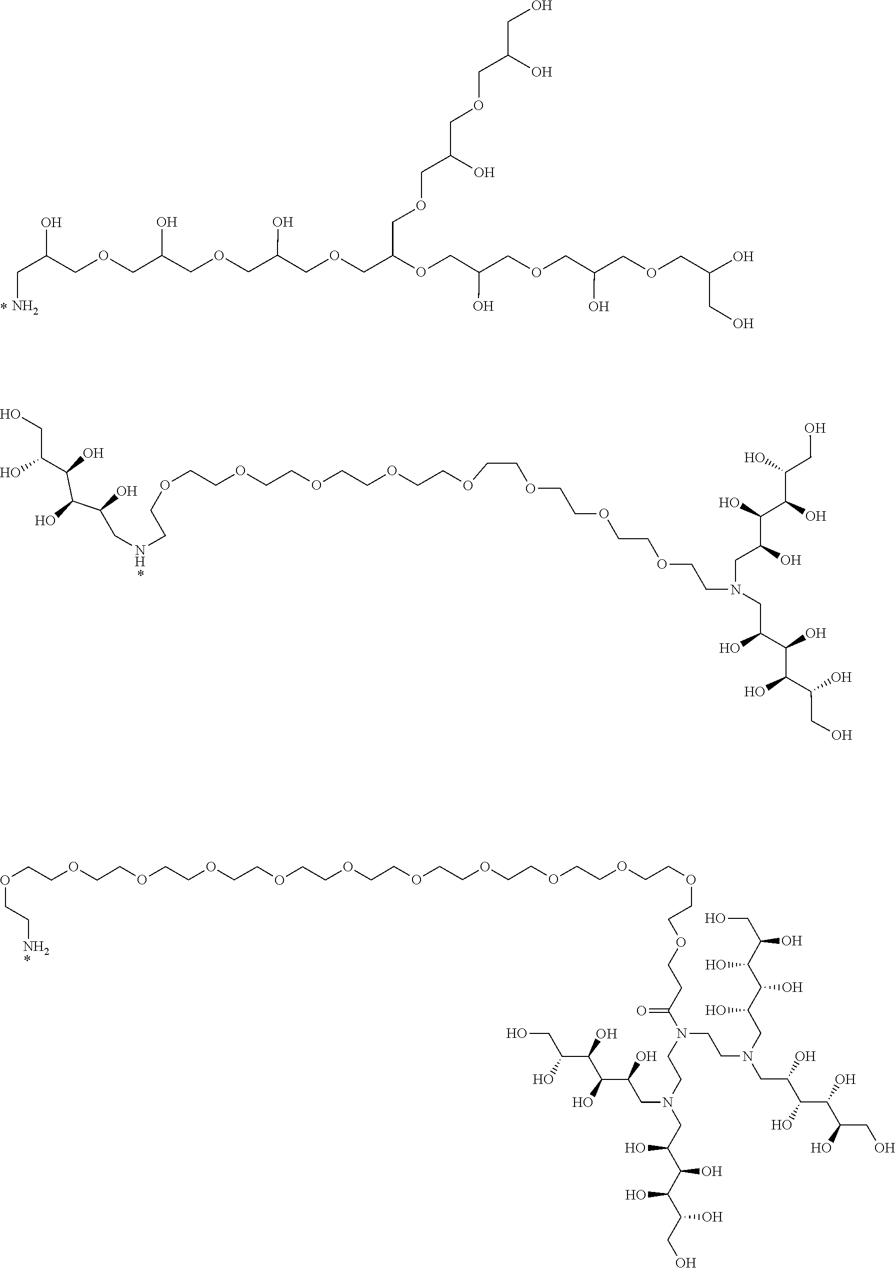

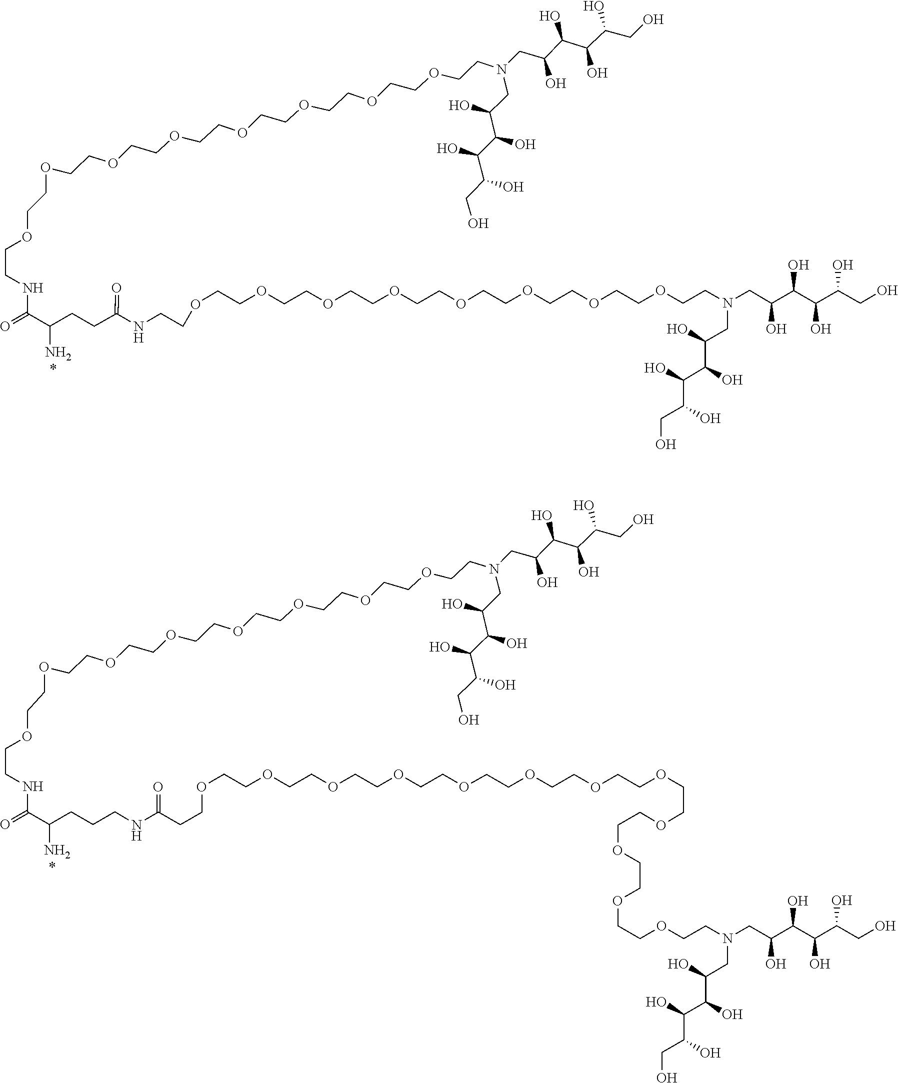

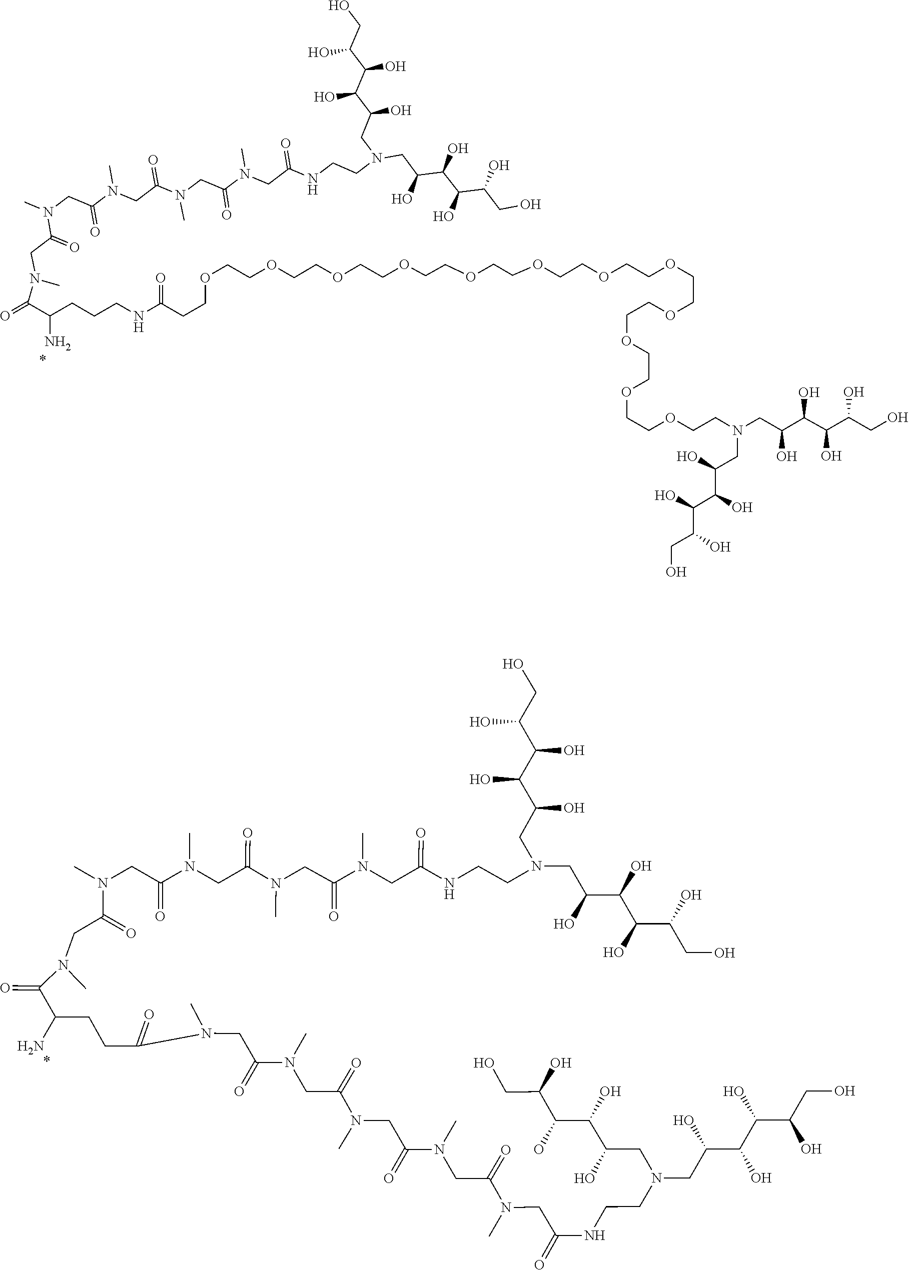

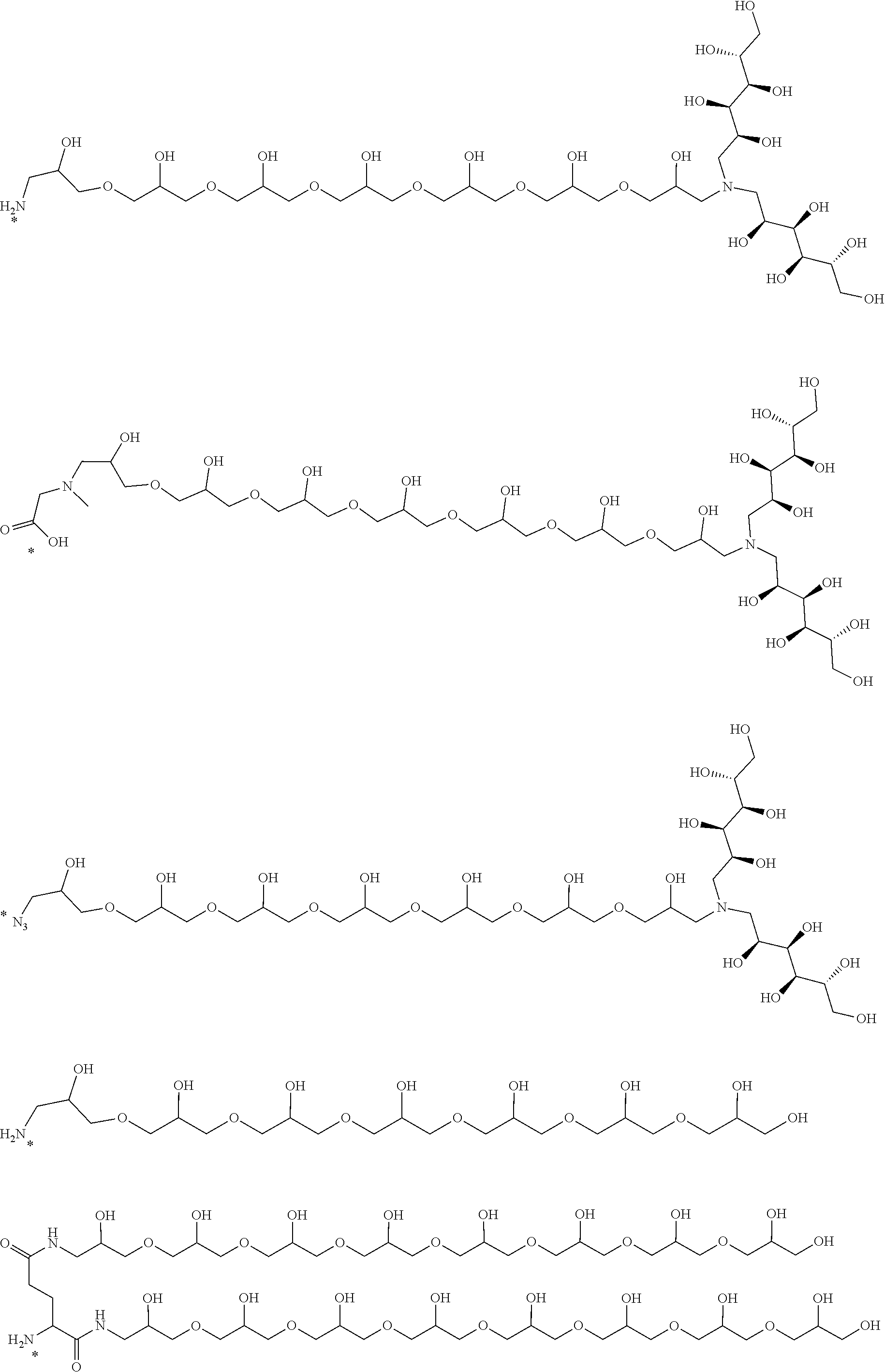

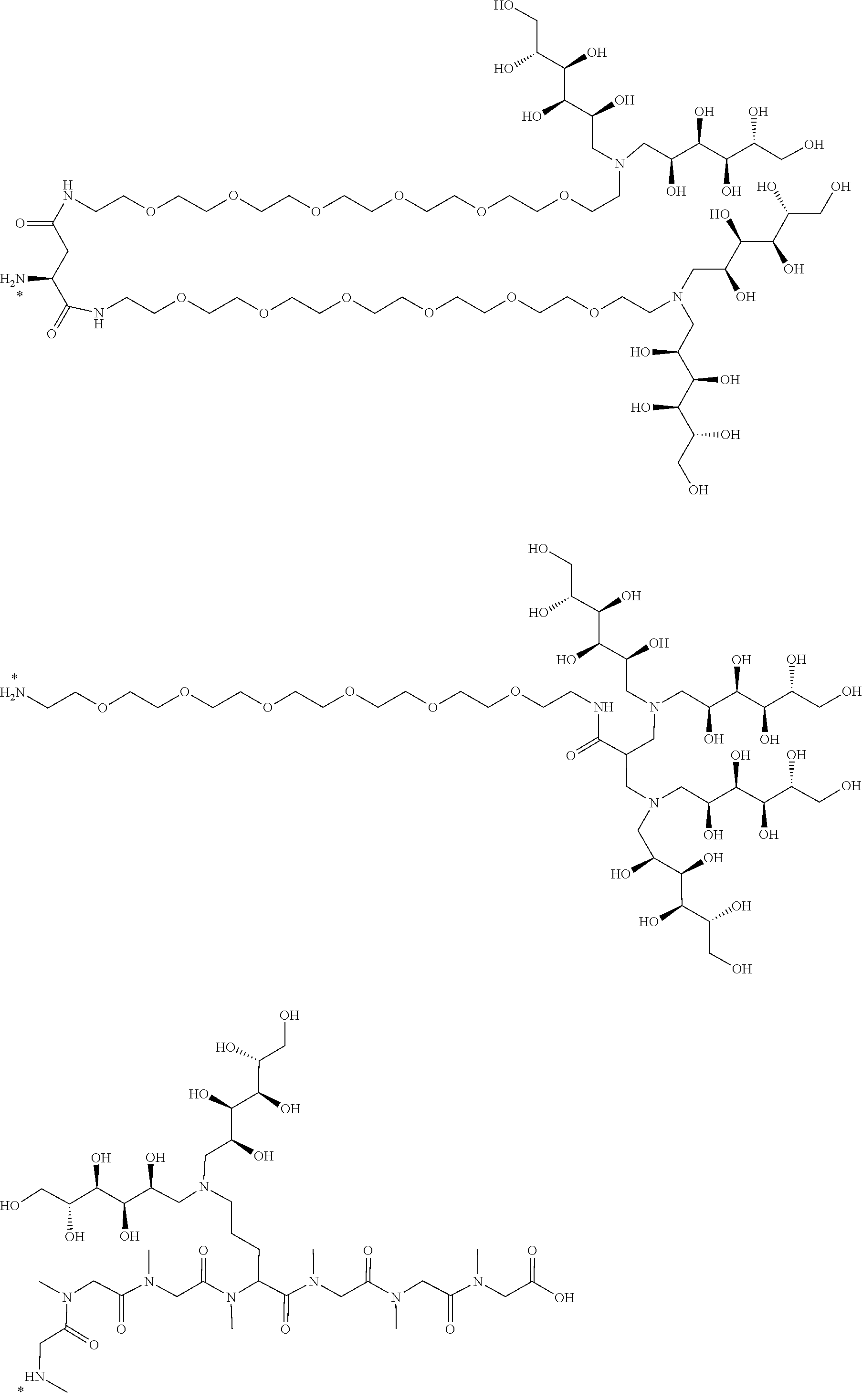

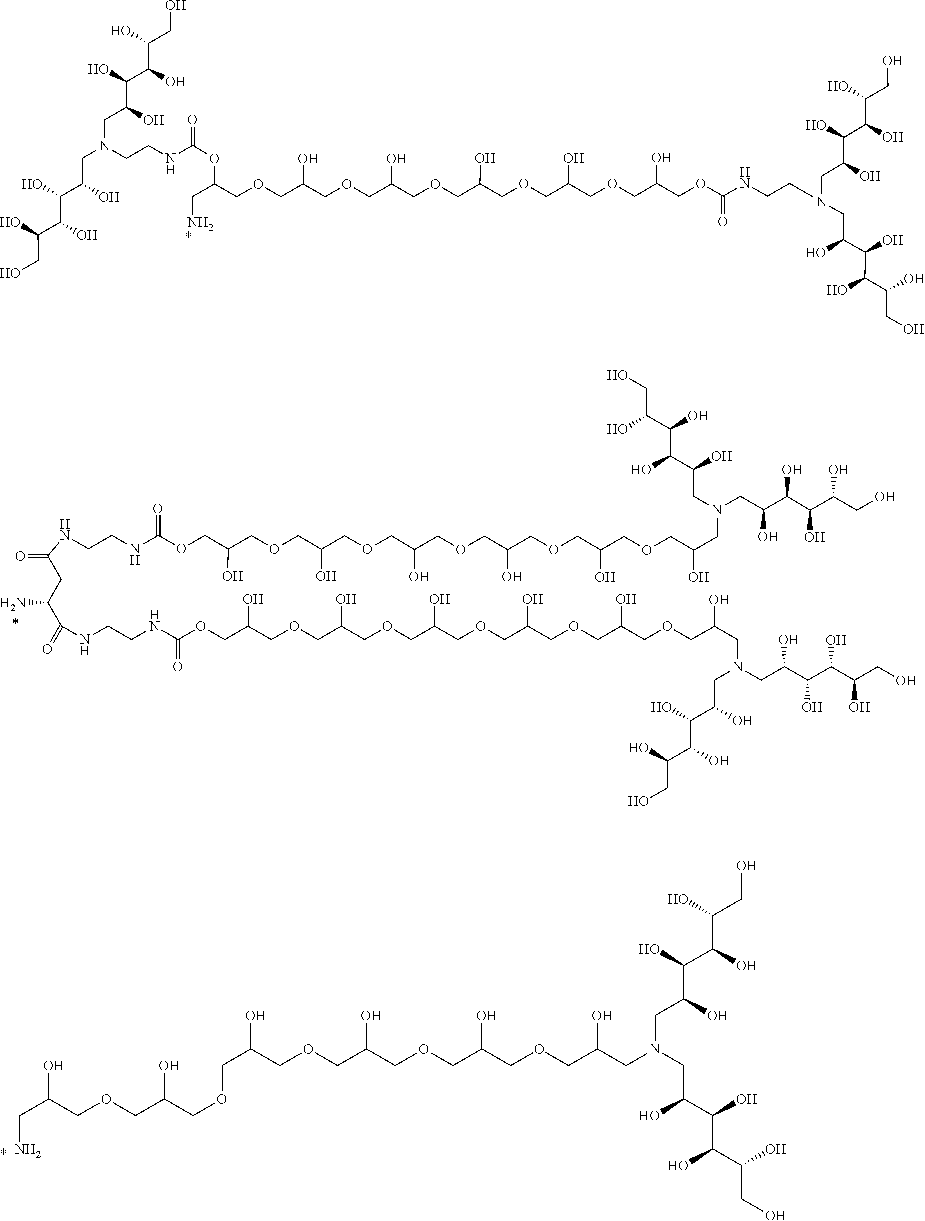

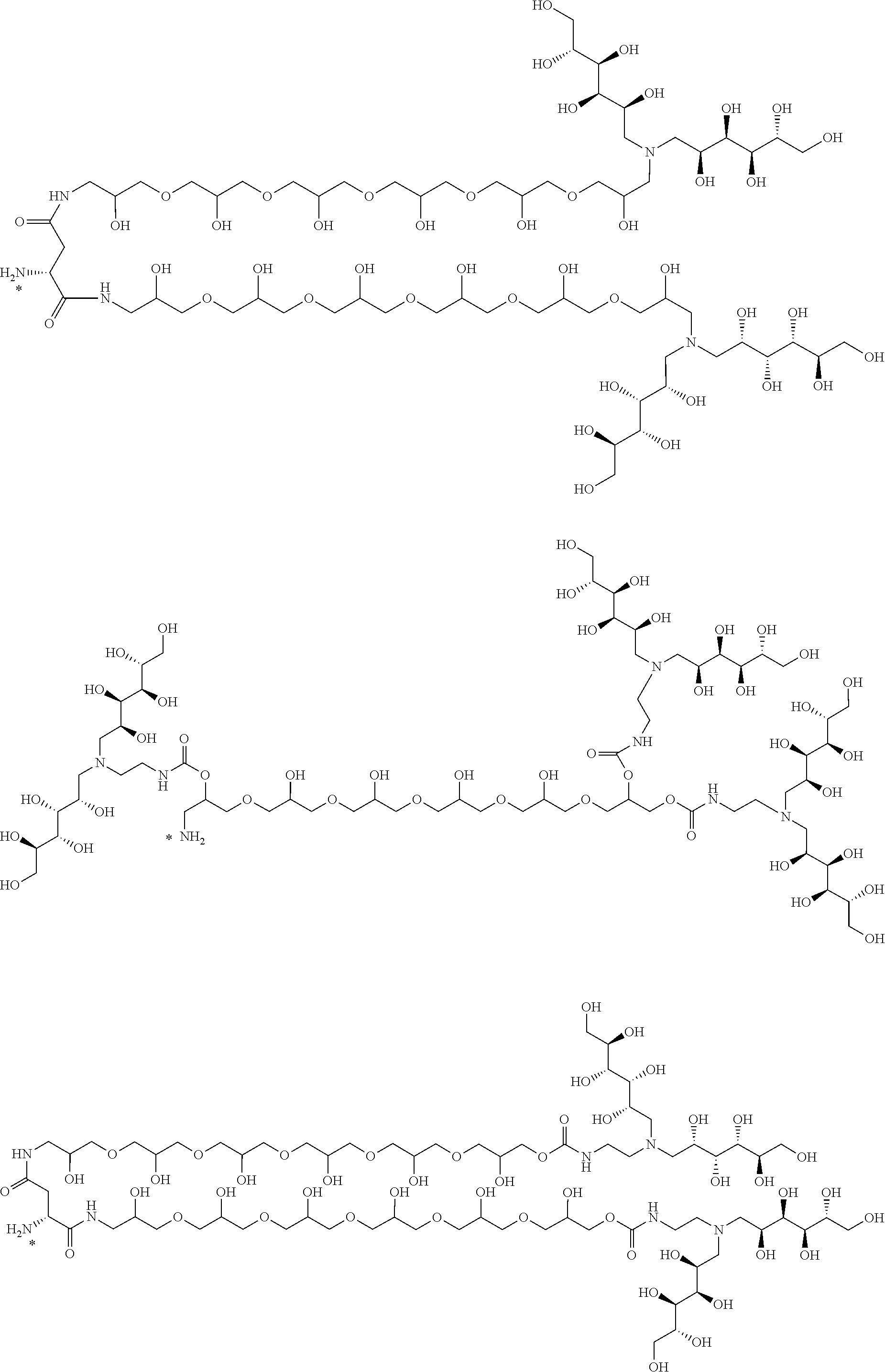

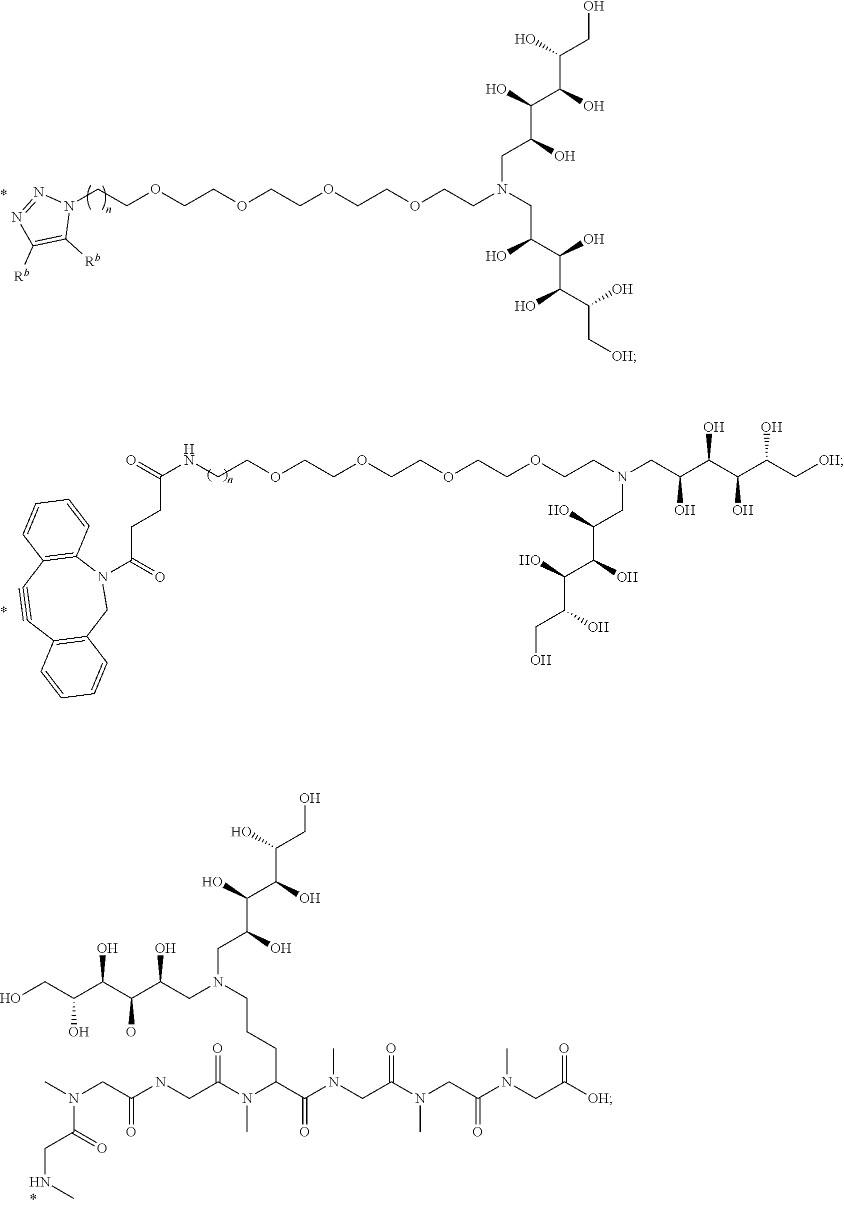

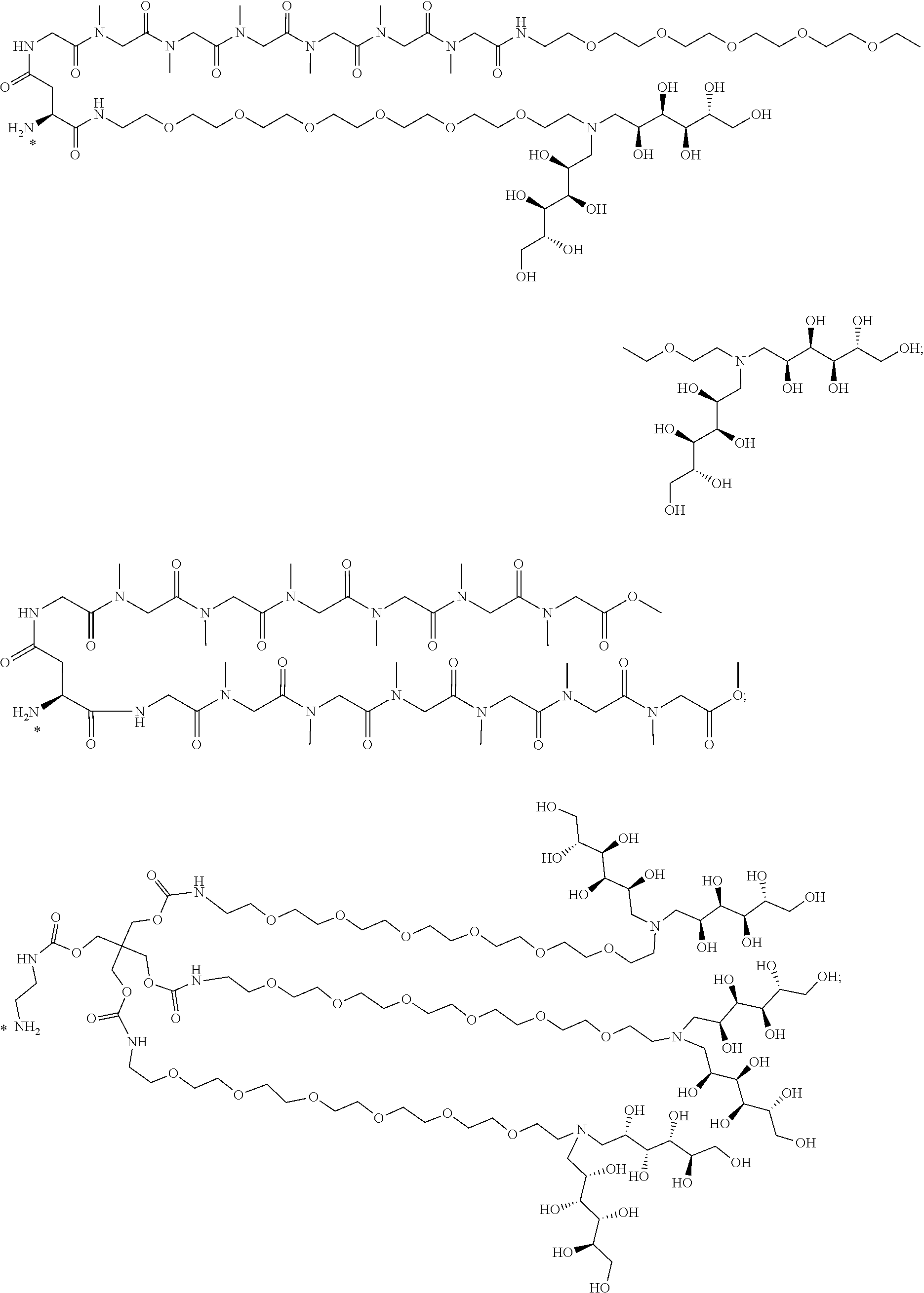

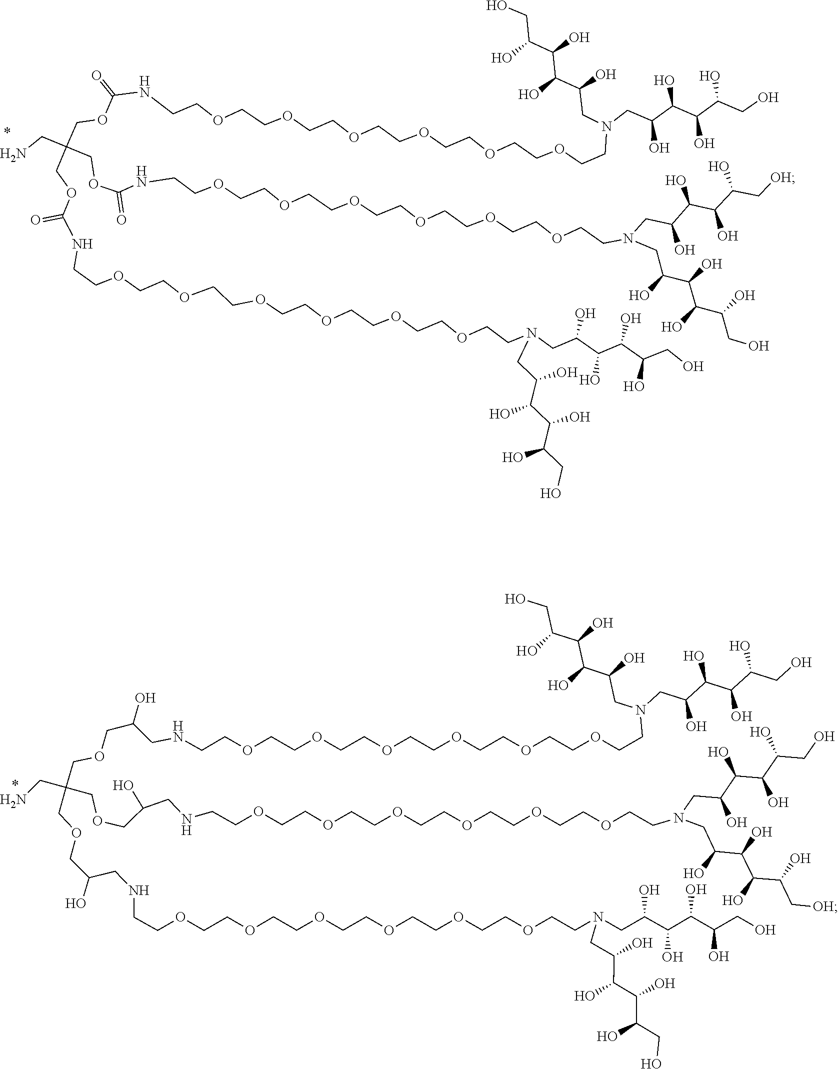

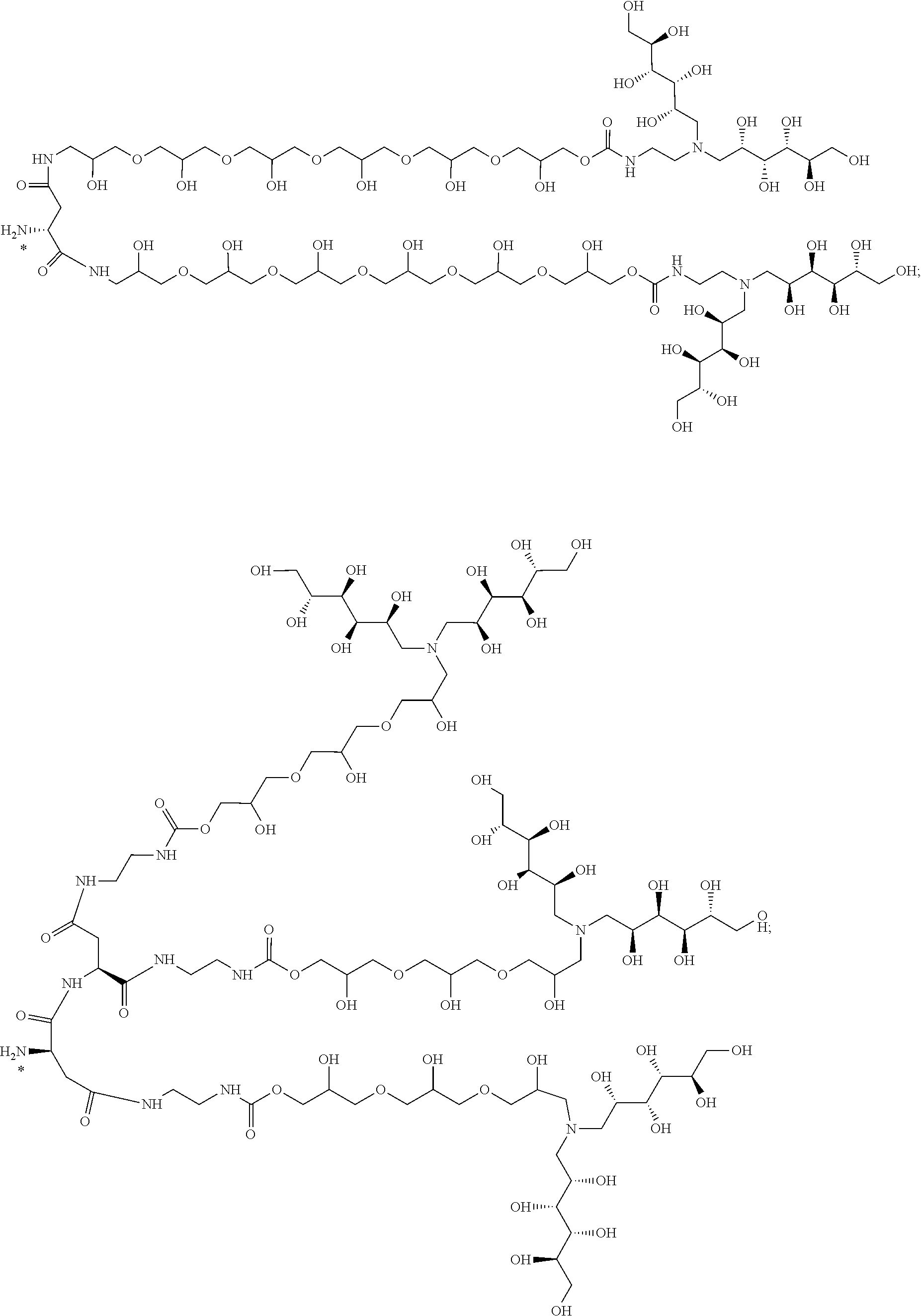

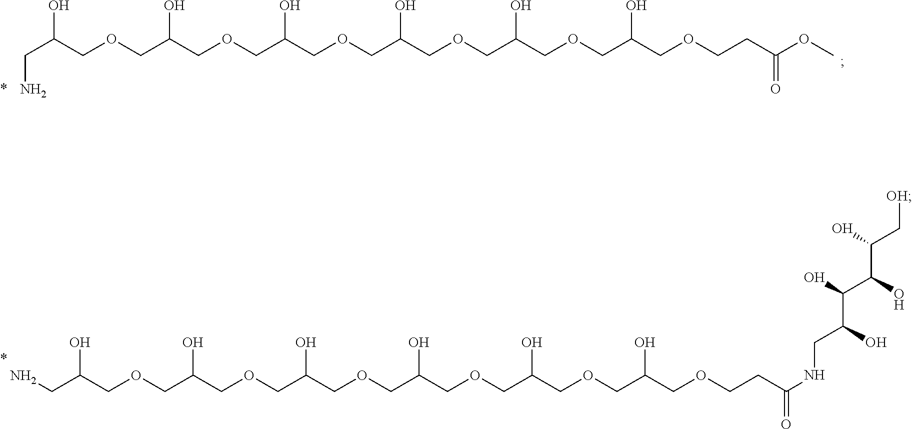

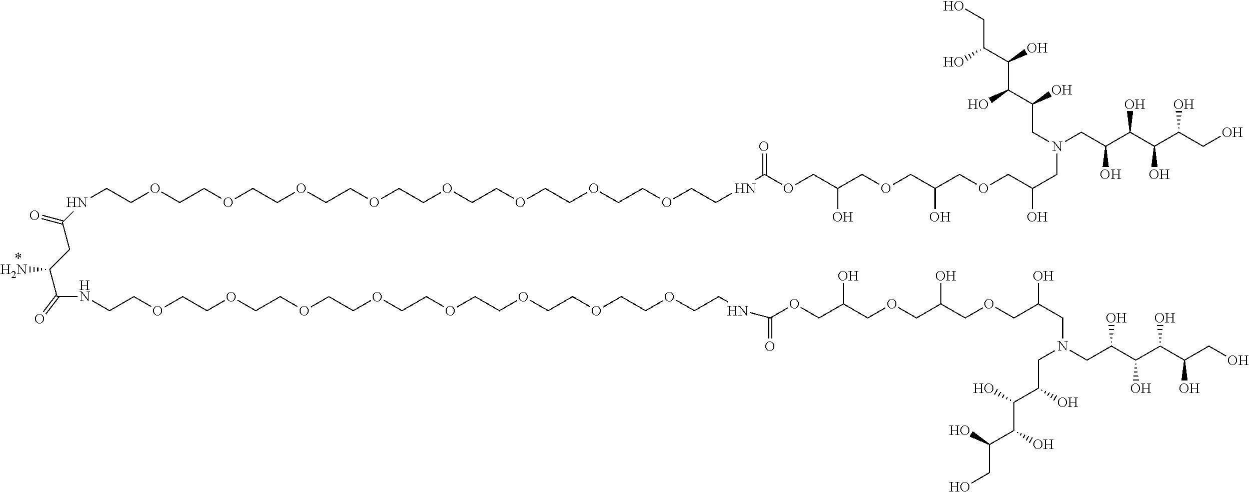

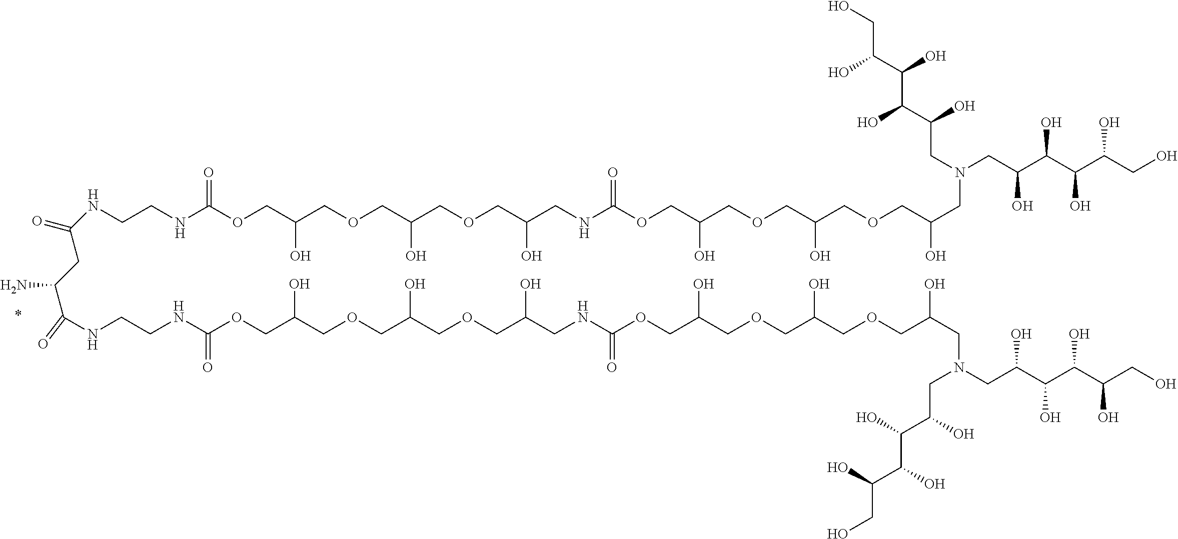

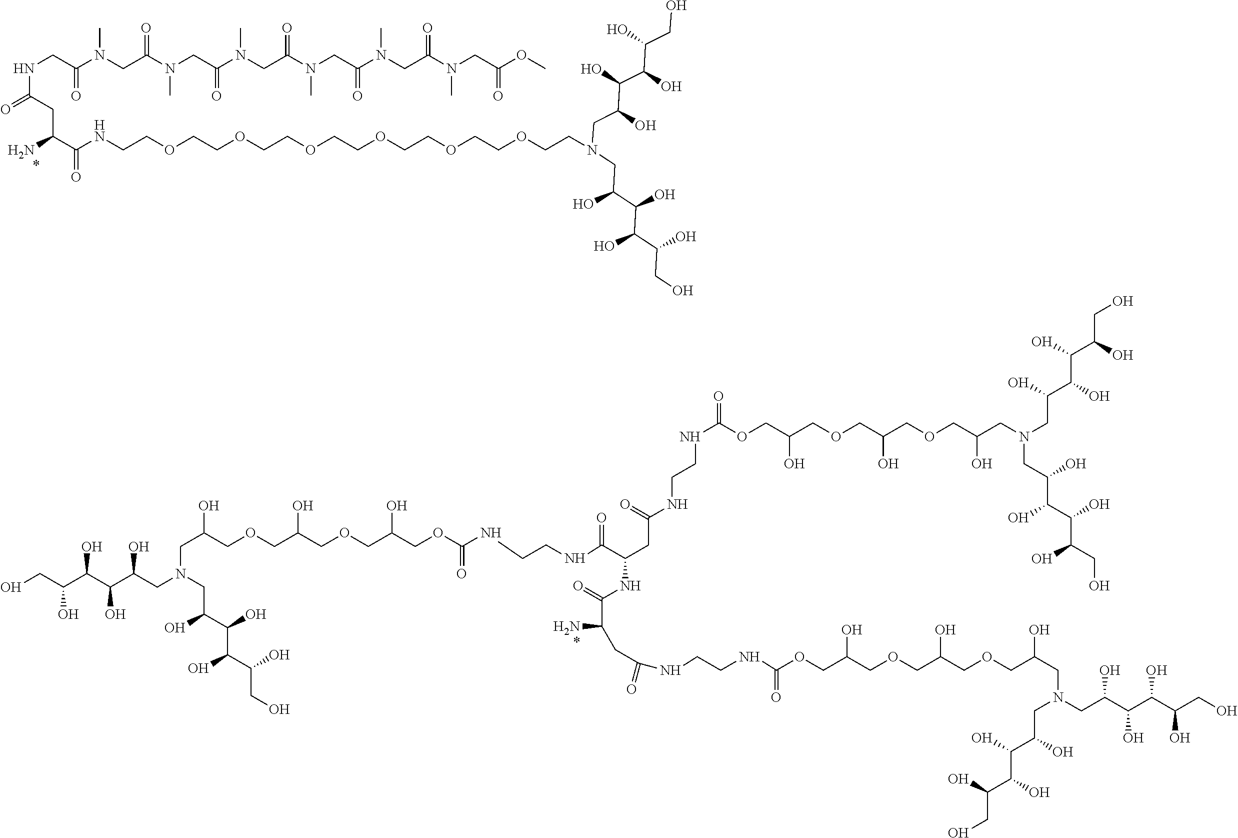

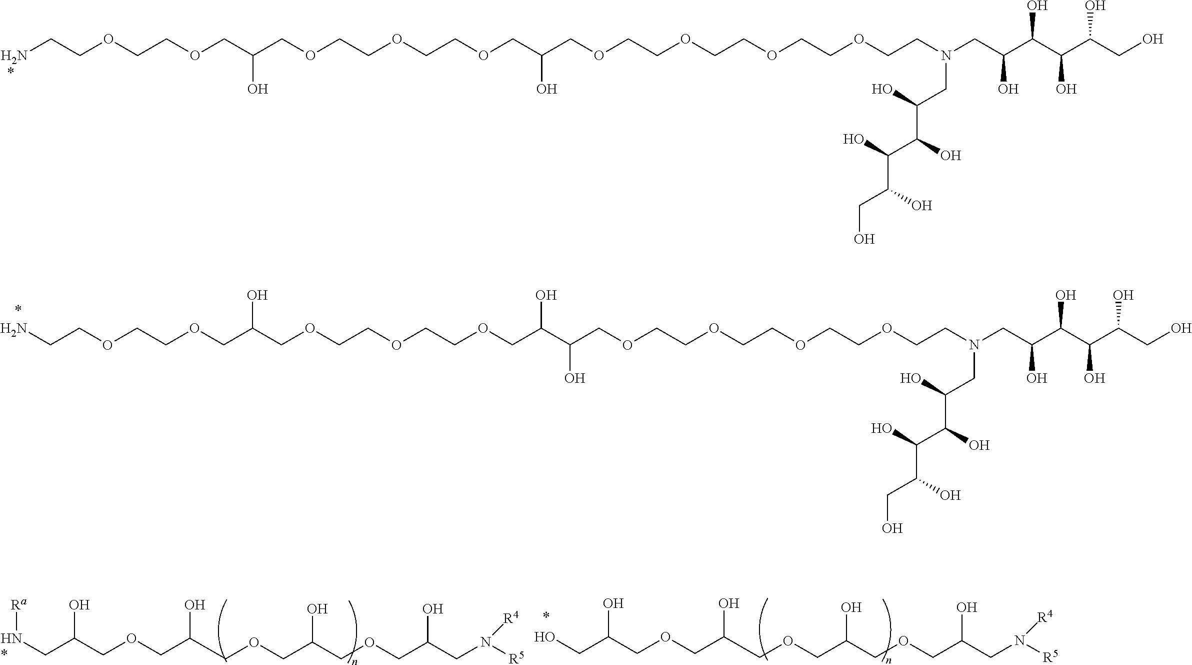

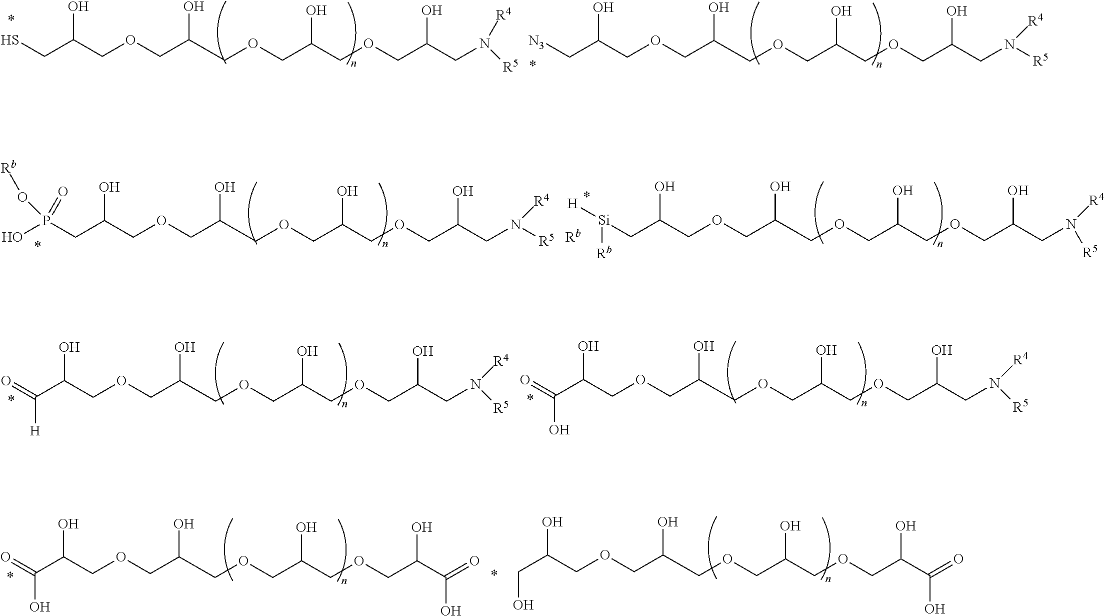

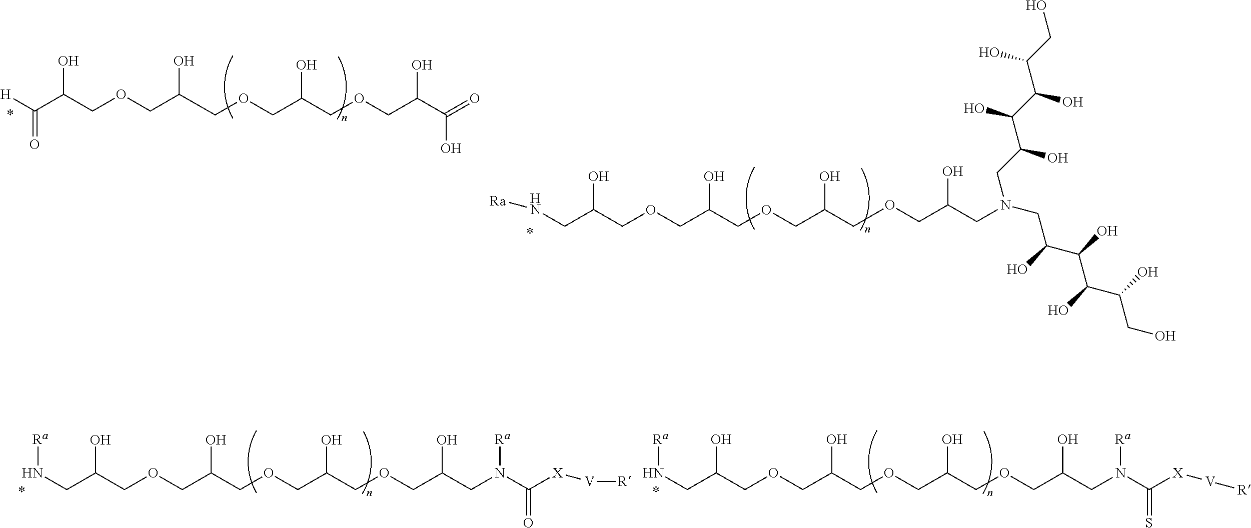

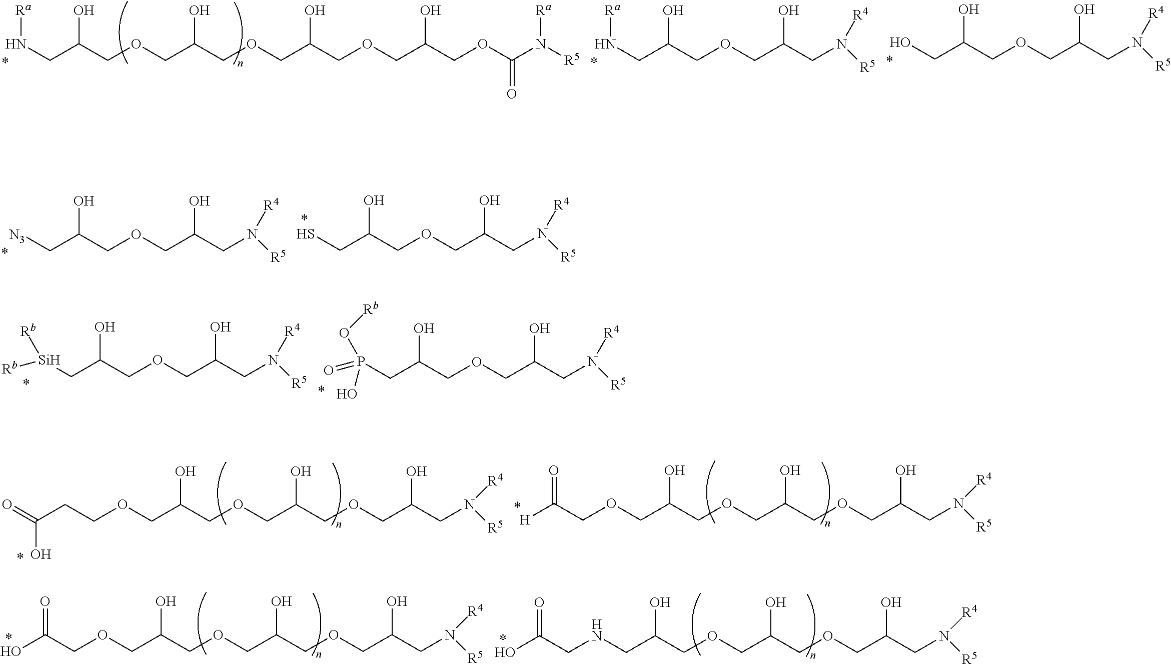

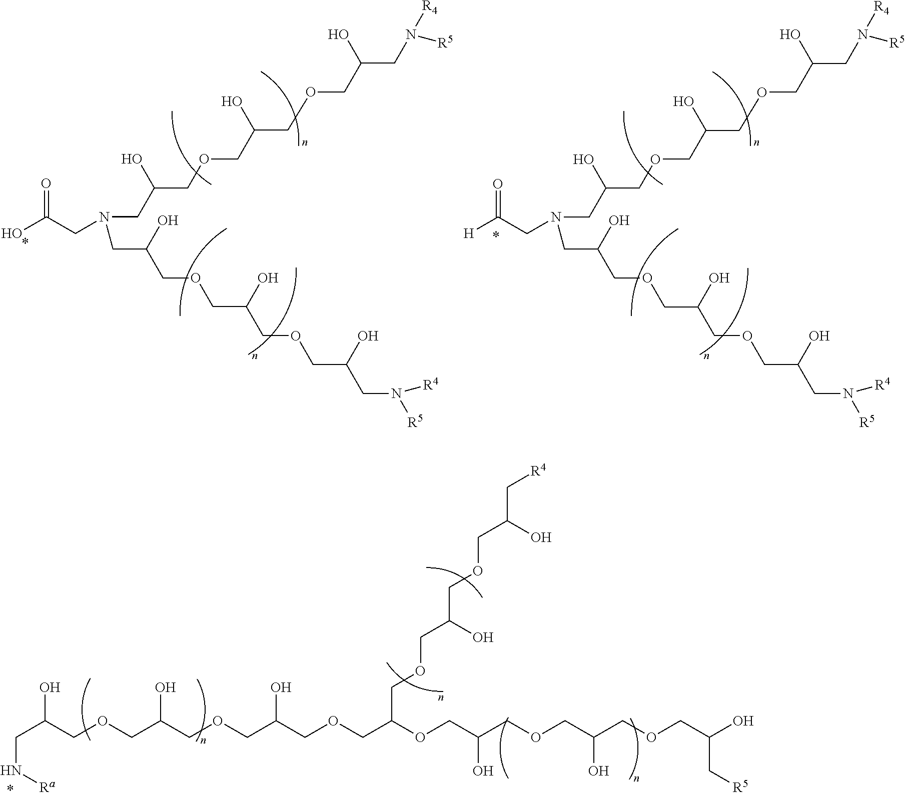

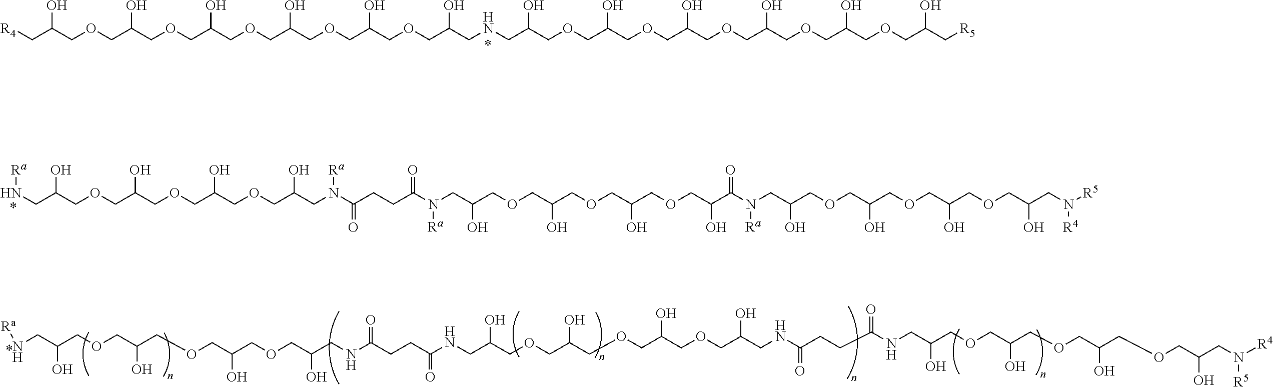

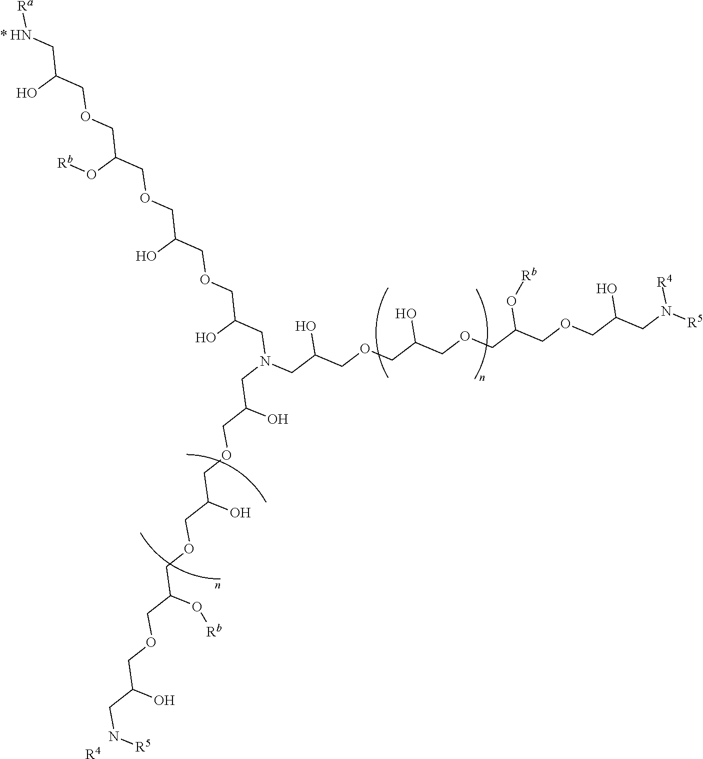

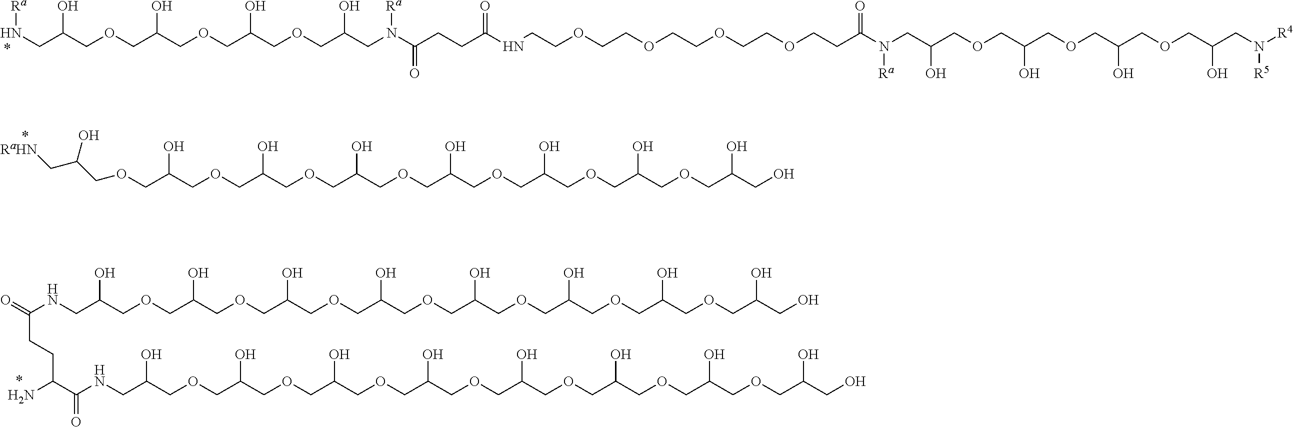

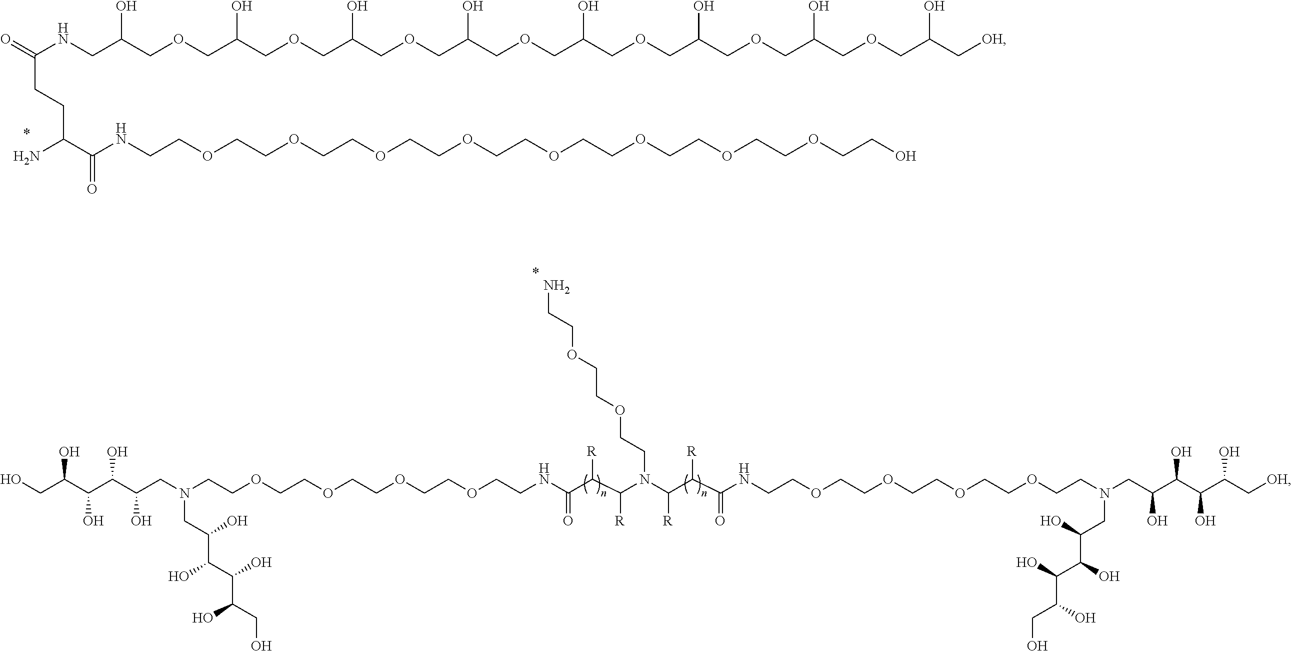

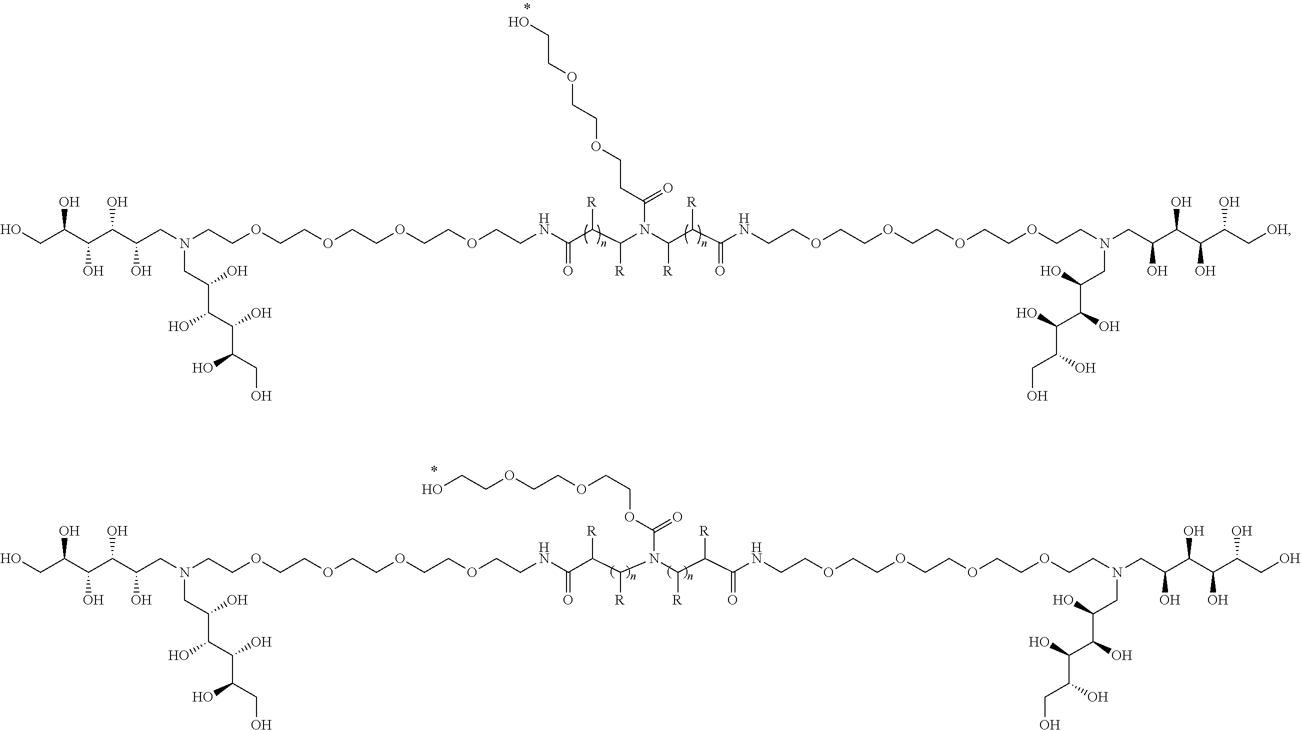

or a stereoisomer thereof, wherein:
   (*) indicates the attachment site to an Amino Acid unit;
   each R, R^{a} and R is independently H or C₁-C₆ alkyl;
   R′ is H, C₁-C₆ alkyl, —N(R⁴)(R⁵) or —CO₂H;
   each n is independently 1 to 12;
   X is O, NR or —CH₂—;
   V is bond or C₁-C₆ alkyl;
   one of R⁴ and R⁵ is selected from a H, polyhydroxyl group, substituted polyhydroxyl group, —C(O)-polyhydroxyl group, substituted —C(O)-polyhydroxyl group, a chelator, or —C(O)—R, where R is a Sugar unit of formula (XII) or (XIII); and the other of R⁴ and R⁵ is selected from H, polyhydroxyl group, substituted polyhydroxyl group, —C(O)-polyhydroxyl group, substituted —C(O)-polyhydroxyl group, a chelator, or —C(O)—R, where R is a Sugar unit of formula (XII) or (XIII), and polyethylene glycol, optionally having 1 to 24 ethylene glycol subunits; or —NR⁴R together from a C₃-C₈ heterocycle, and wherein R⁴ and R⁵ are not both H.

In some embodiments, provided is a Linker compound, comprising a Polar group having a formula selected from:

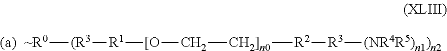

or a stereoisomer or salt thereof, wherein:
   R⁰ is a functional group for attachment to a subunit of the Amino Acid unit;
   R¹ and R² are each, independently, a bond or C₁-C₆ alkylene;
   each R³ is independently selected from a bond, C₁-C₁₂ alkylene, —OC₁-C₁₂ alkylene, —C(═O)—, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, C₁-C₁₂alkylene-NH—C(O)—, -heteroarylene, heteroaryl-C₁-C₁₂ alkylene, heteroaryl-C₁-C₁₂ alkylene-C(O)—, and —C(O)NR⁷R⁸, wherein one of R⁷ and R⁸ is H or C₁-C₁₂ alkylene and the other is C₁-C₁₂ alkylene;
   R⁴ and R⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, and wherein R⁴ and R⁵ are not both H;
   each R⁶ is independently selected from —NR^{a}—, —NR^{a}—C₁-C₆alkylene-NR^{a}—, —NR^{a}—C(O)—NR^{a}—S(O)₂—NR^{a}— or —NR^{a}—C(O)—C₁₋₆alkylene-;
   each R^{a} is independently selected from H, C₁-C₆ alkyl, or polyhydroxyl group;
   each n0 is independently 2 to 26;
   n1 is 1 to 6; and
   n2 is Ito 6;

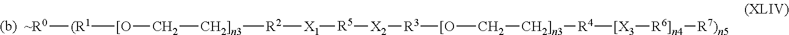

or a stereoisomer or salt thereof, wherein:
   R⁰ is a functional group for attachment to a subunit of the Amino Acid unit;
   R¹, R², R³ and R⁴ are each, independently, a bond or C₁-C₆ alkylene;
   X₁, X₂ and X₃ are each independently —NR^{N}—C(O)— or —C(O)—NR^{N}—;
   each R^{N} independently represents H, C₁-C₆ alkyl, or polyhydroxyl group;
   R and R⁶ each independently represent a bivalent polyhydroxyl group;
   R is H, OH or C₁-C₆ alkyl;
   each n3 is independently 0 to 26, with the proviso that at least one n3 is 2 to 26;
   n4 is 0 to 10; and
   n5 is 1 or 2; or

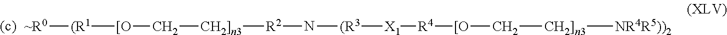

or a stereoisomer or salt thereof, wherein:
   R⁰ is a functional group for attachment to a subunit of the Amino Acid unit;
   R¹, R³ and R⁴ are each, independently, a bond or optionally-substituted C₁-C₆ alkylene;
   each R² is independently a bond, C₁-C₆ alkylene, —C(O)— or —O—C(O)—;
   each X₁ is independently —NR^{N}—C(O)— or —C(O)—NR^{N}—;
   each R^{N} independently represents H, C₁-C₆ alkyl, or polyhydroxyl group;
   R⁴ and R⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, and wherein R⁴ and R are not both H; and
   each n3 is independently 2 to 26.

In some embodiments, provided is a Linker compound, comprising a Polar group having one of the following structures prior to attachment to the Amino Acid unit:

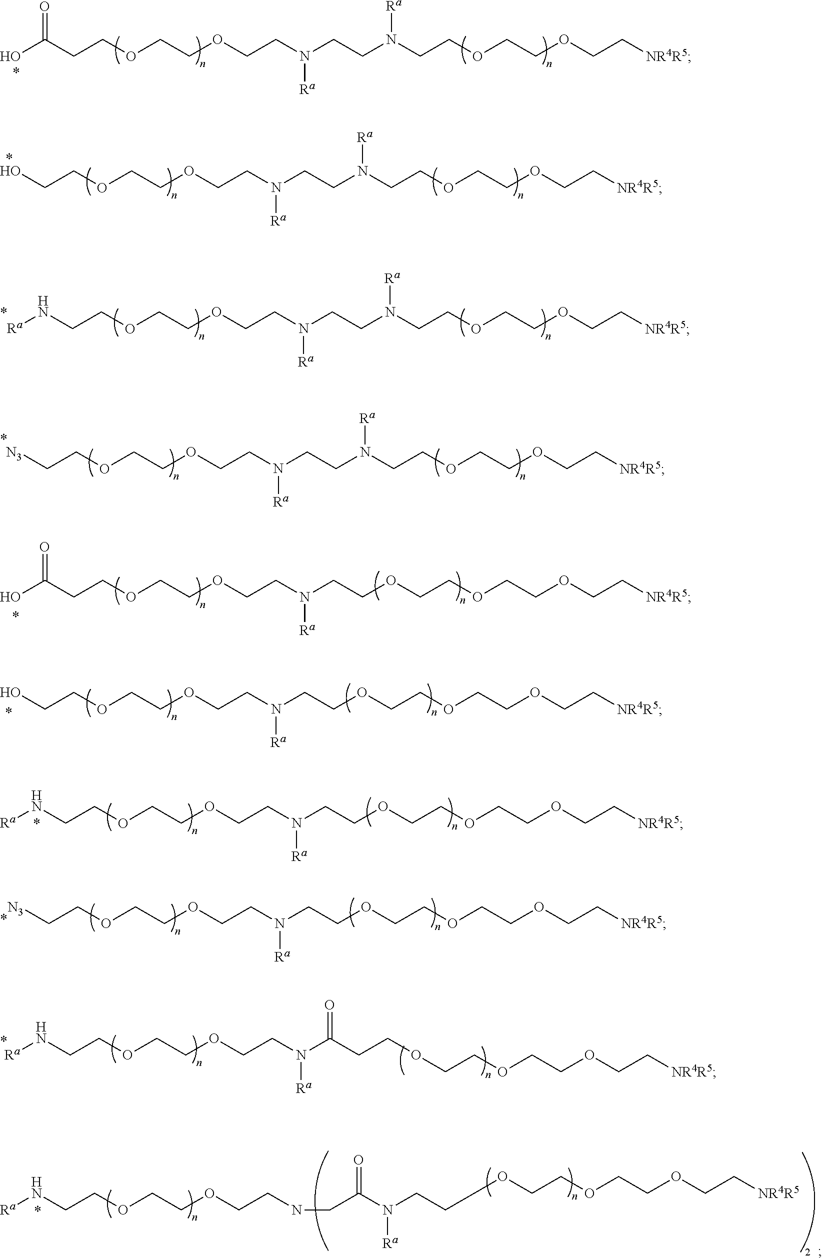

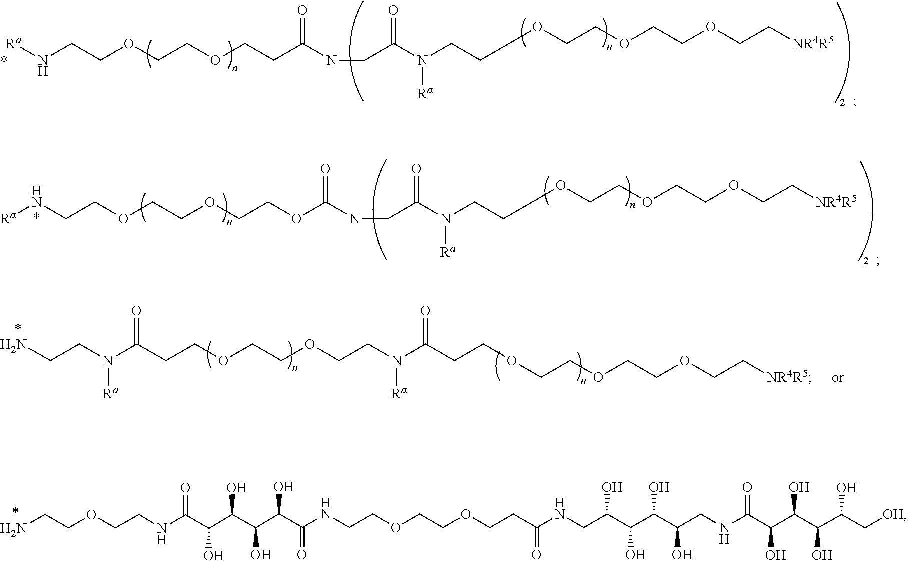

wherein:
   (*) indicates the attachment site to an Amino Acid unit;
   each R^{a} is independently H, alkyl or polyhydroxyl group;
   R⁴ and R⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, and wherein R⁴ and R⁵ are not both H; and
   each n is independently 1 to 12.

In some embodiments, provided is a Linker compound, comprising a Polar group having a formula selected from:

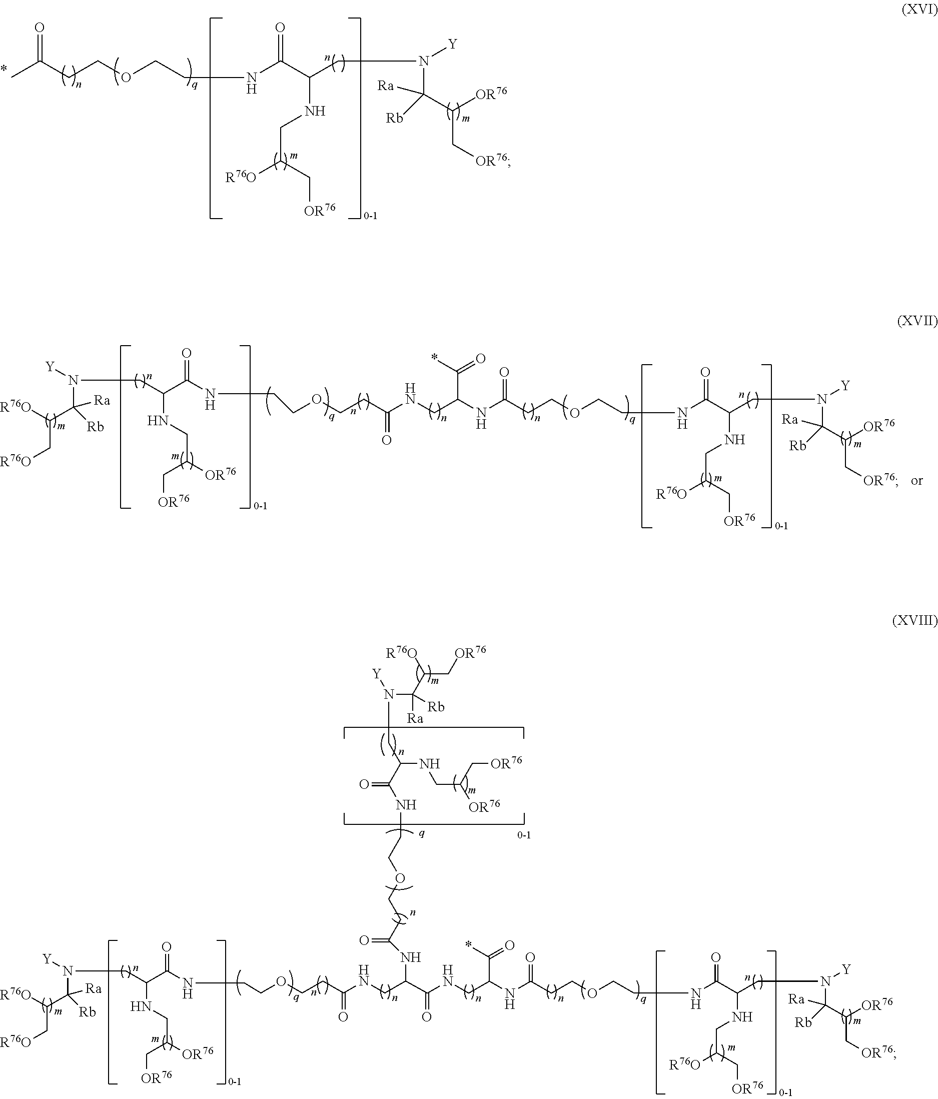

or a stereoisomer or salt thereof, wherein:
   each Y is independently R⁷⁶ or

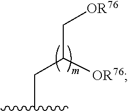

each R⁷⁶ is independently H, acetyl, —P(═O)(OH)₂, or —(CH₂)ᵥ—O—S(═O)₂(OH);
each Rₐ and R_{b} is independently H or Rₐ and R_{b} are taken together with the carbon to which they are attached to form an oxo group;
each q is independently 2-26;
each m is independently 1 to 4;
each n is independently 1 to 4;
each v is independently 1 to 6; and
each * is an attachment to an Amino Acid unit.

In some embodiments, provided is a Linker compound, comprising a Polar group having a formula selected from:

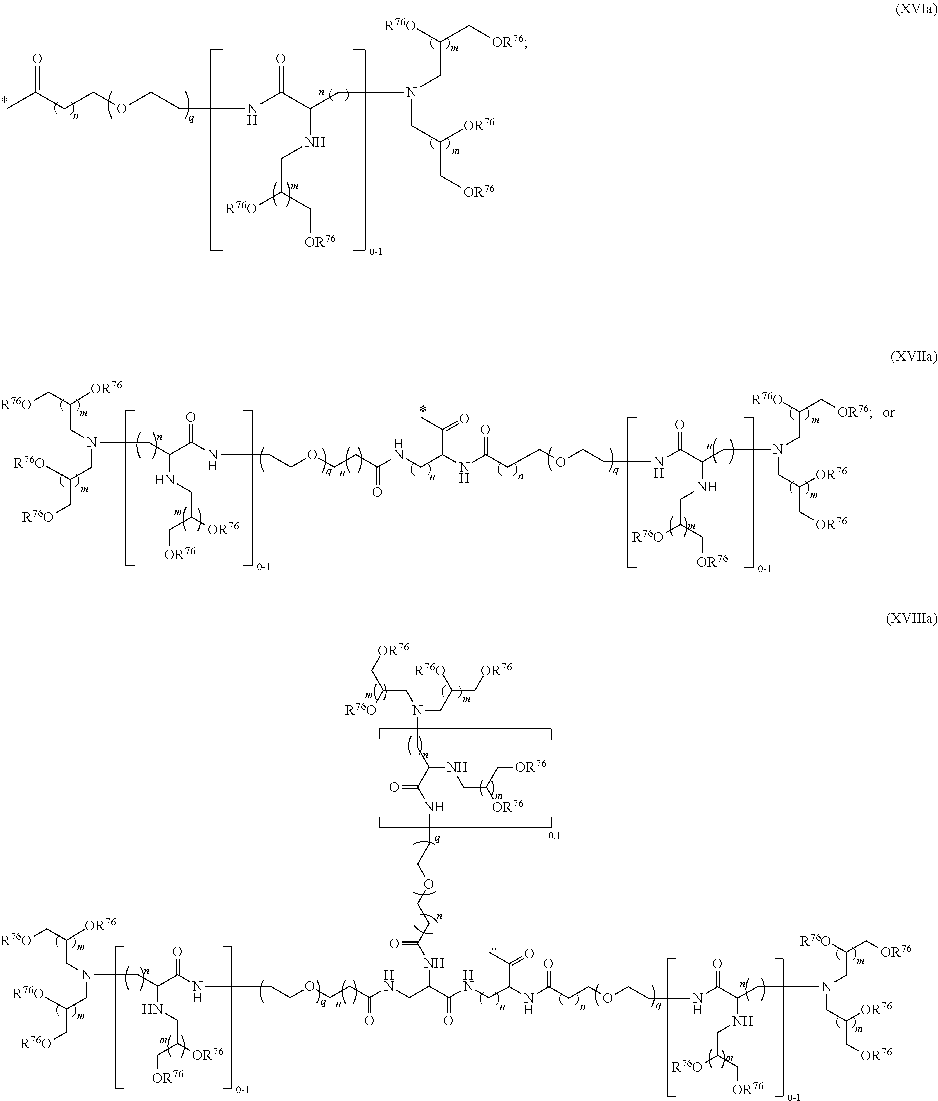

or a stereoisomer or salt thereof, wherein:
   each R⁷⁶ is independently H, acetyl, —P(═O)(OH)₂, or —(CH₂)ᵥS(═O)₂(OH);
   each q is independently 2-26;
   each m is independently 1 to 4;
   each n is independently 1 to 4;
   each v is independently 1 to 6; and
   each * is an attachment to an Amino Acid unit.

In some embodiments, provided is a Linker compound, comprising a Polar group having a formula selected from:

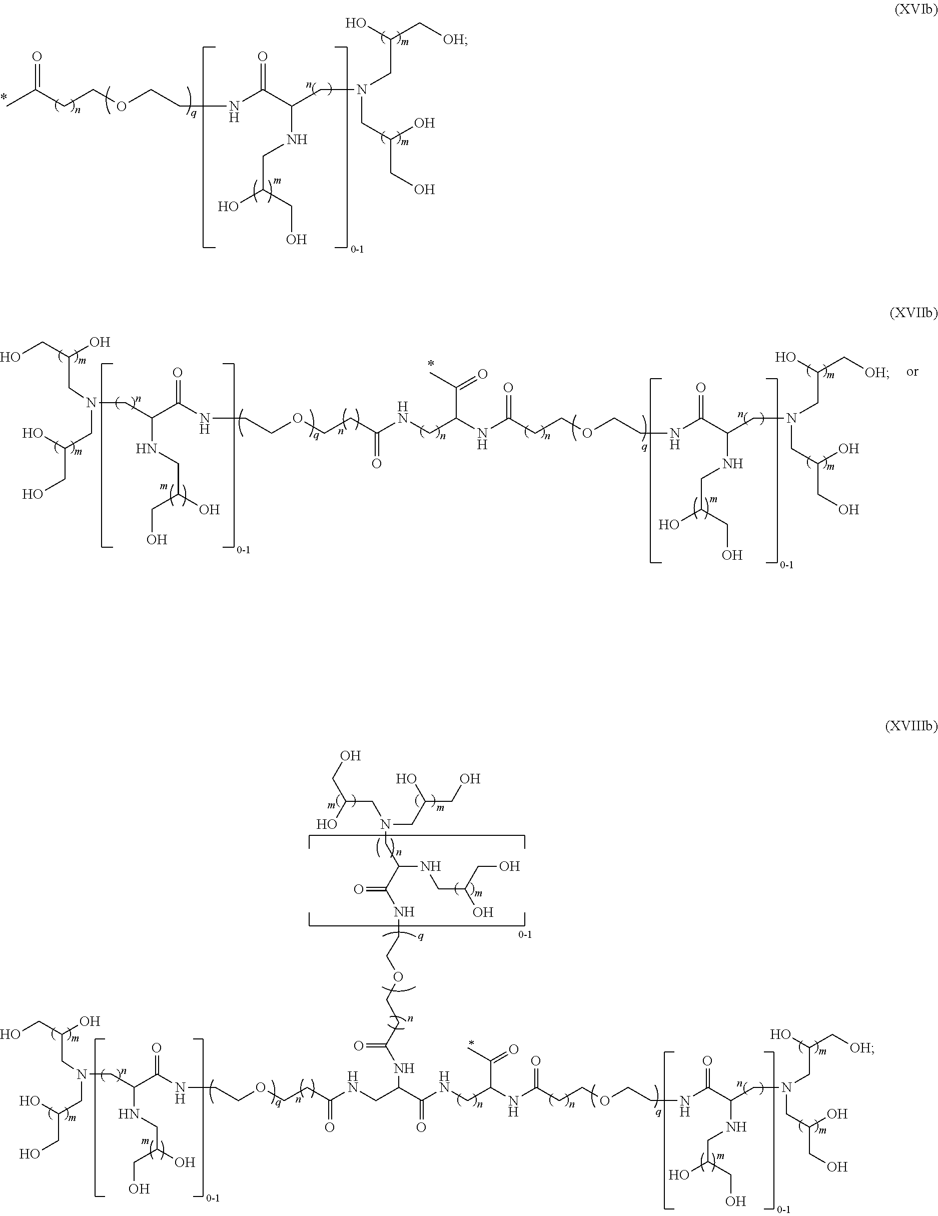

or a stereoisomer or salt thereof, wherein:
   each q is independently 2-26;
   each m is independently 1 to 4;
   each n is independently 1 to 4; and
   each * is an attachment to an Amino Acid unit.

In some embodiments, provided is a Linker compound, wherein Y is R⁷⁶.

In some embodiments, provided is a Linker compound, wherein Y is

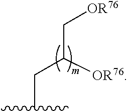

In some embodiments, provided is a Linker compound, wherein each Rₐ and R_{b} is independently H.

In some embodiments, provided is a Linker compound, wherein Rₐ and R_{b} are taken together with the carbon to which they are attached to form an oxo group.

In some embodiments, provided is a Linker compound, wherein q is 10-20.

In some embodiments, provided is a Linker compound, wherein q is 12.

In some embodiments, provided is a Linker compound, wherein the Polar group has one of the following structures prior to attachment to the Amino Acid unit:

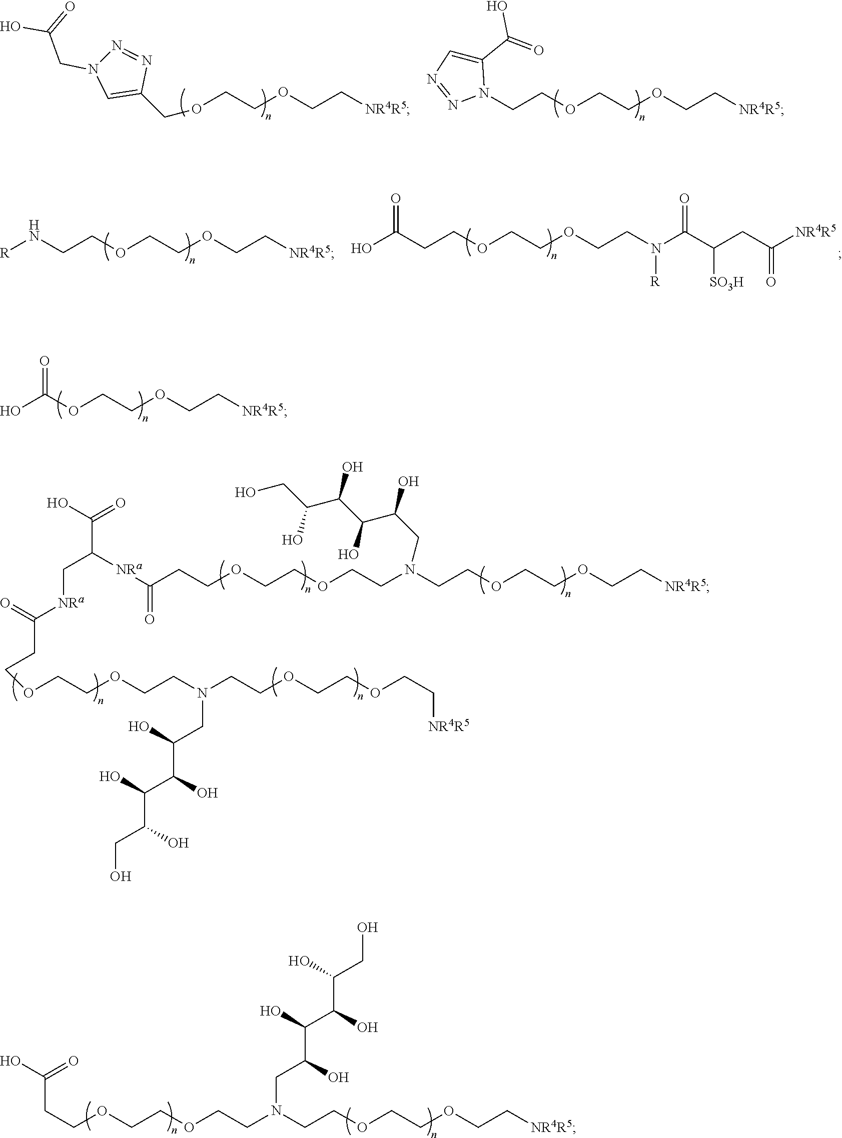

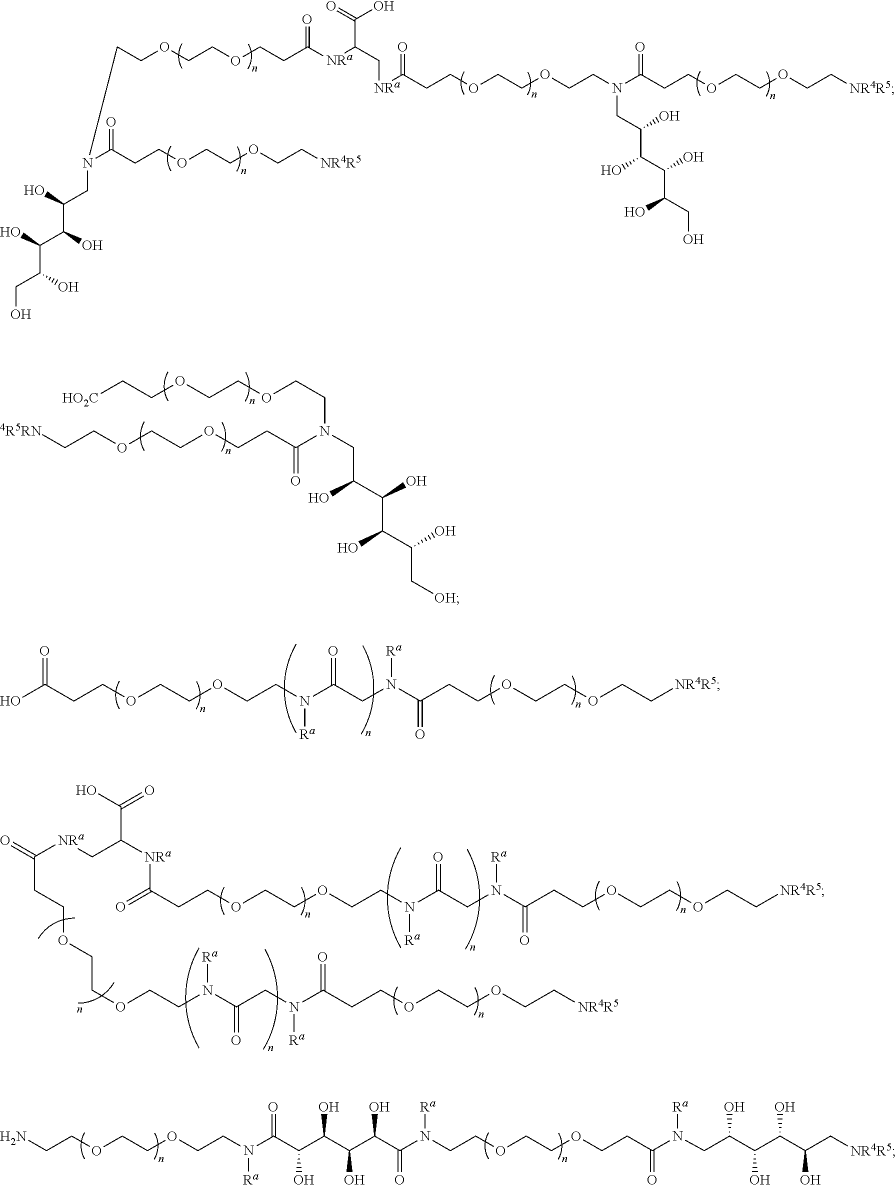

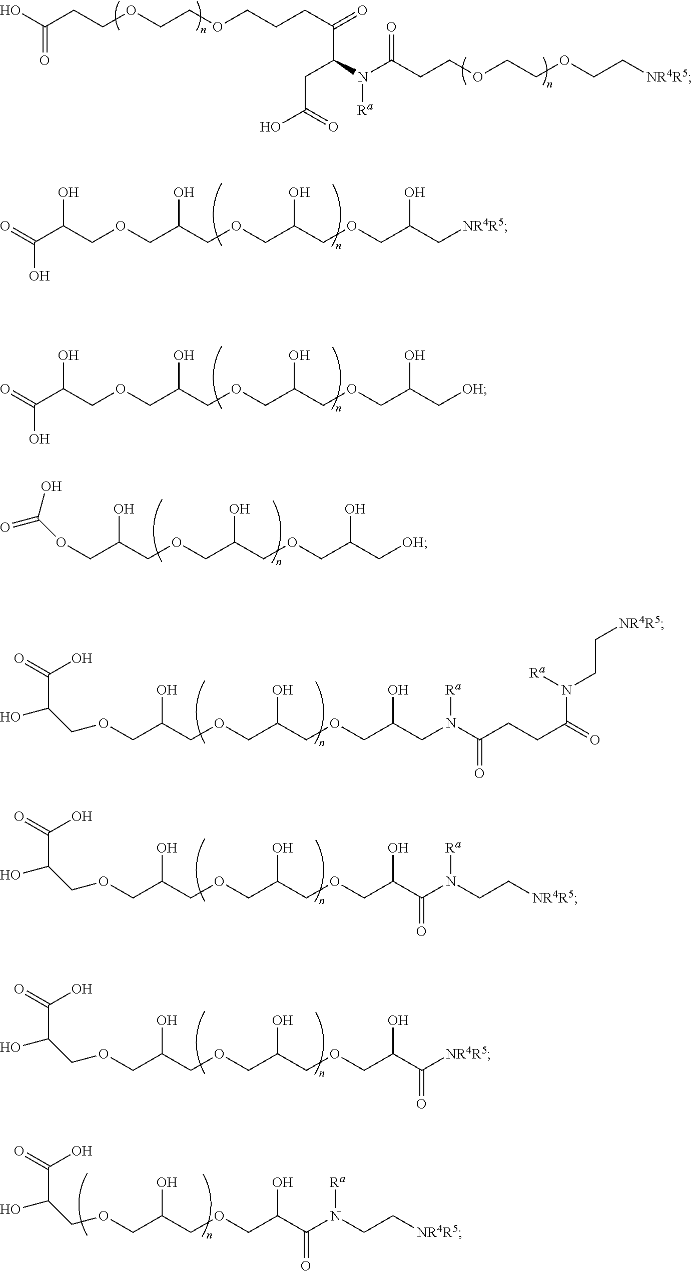

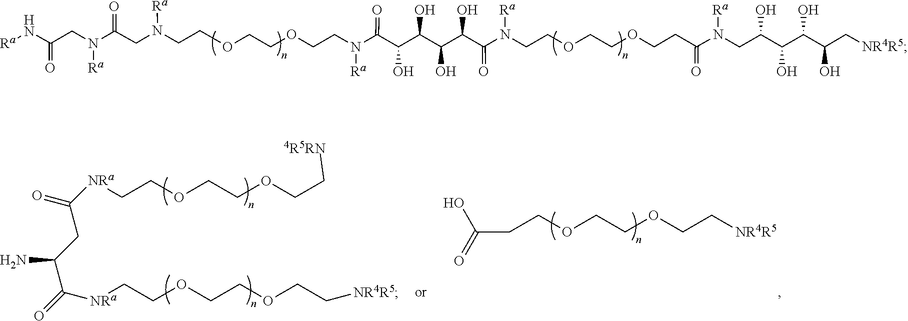

or a stereoisomer thereof, wherein R^{a} is H or C₁₋₆ alkyl and n is 1-20.

In some embodiments, provided is a Linker compound, wherein the Polar group has one of the following structures prior to attachment to the Amino Acid unit:

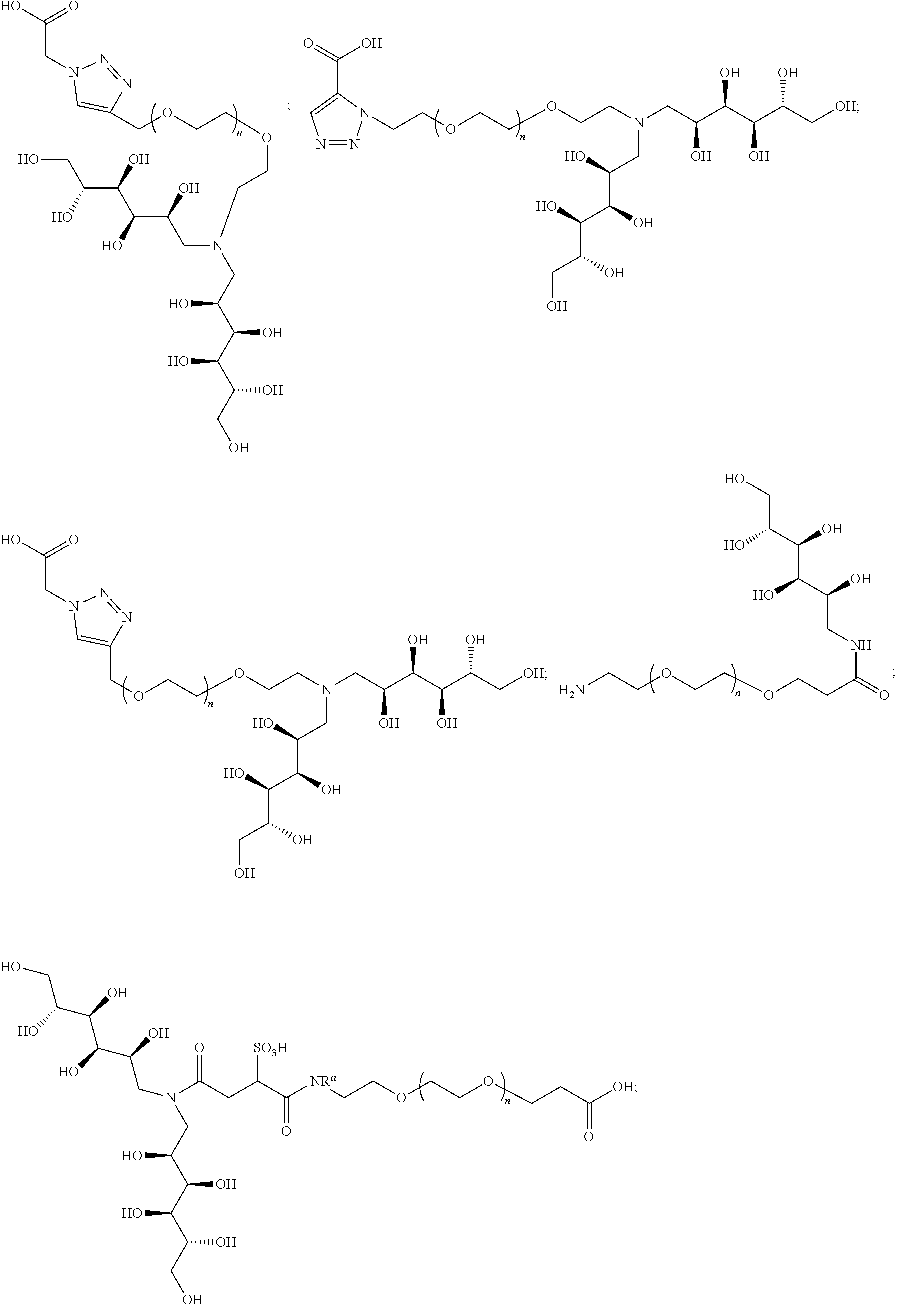

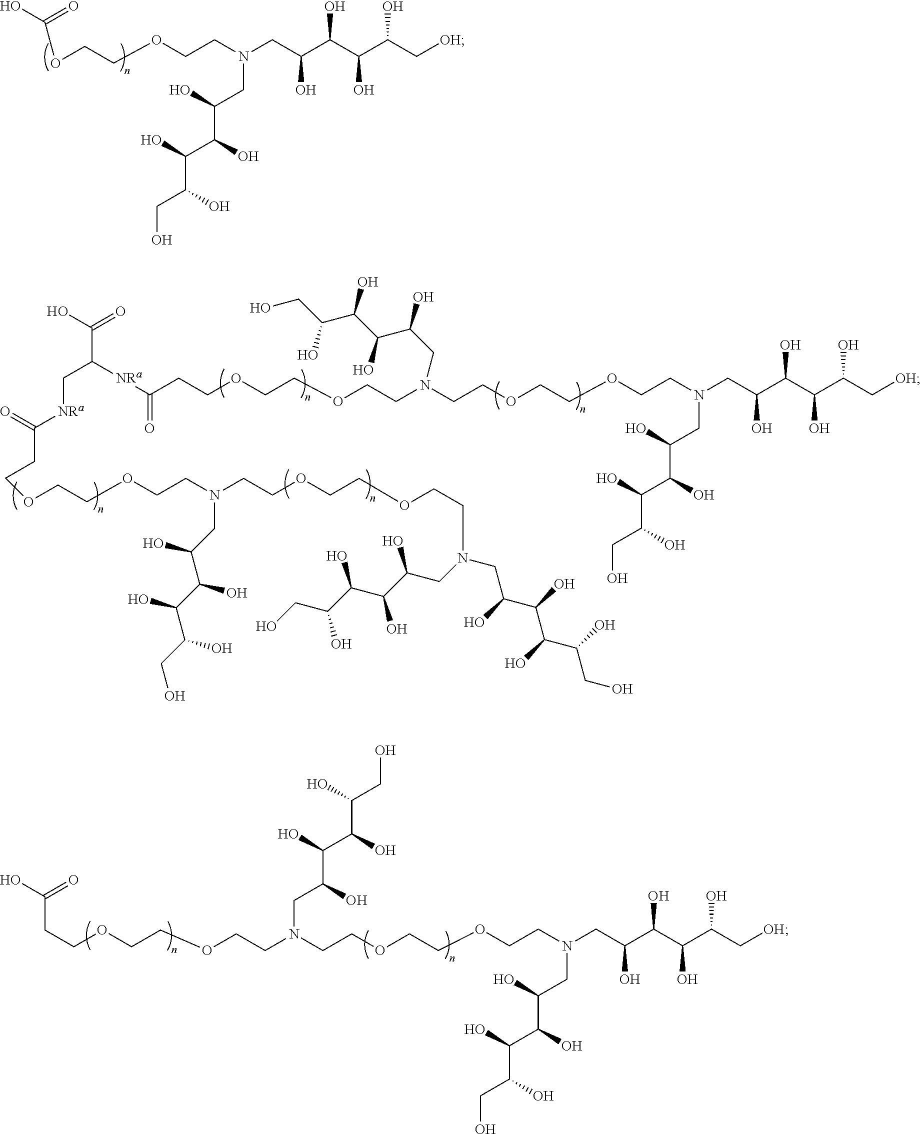

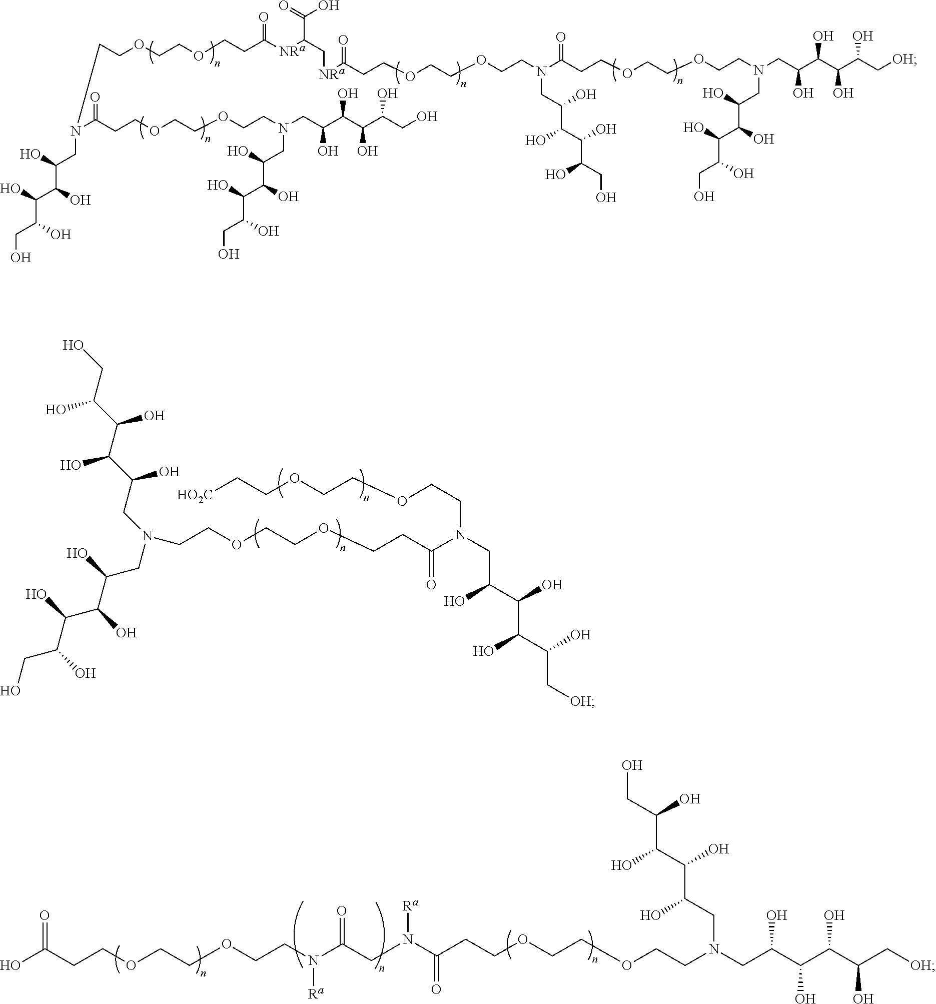

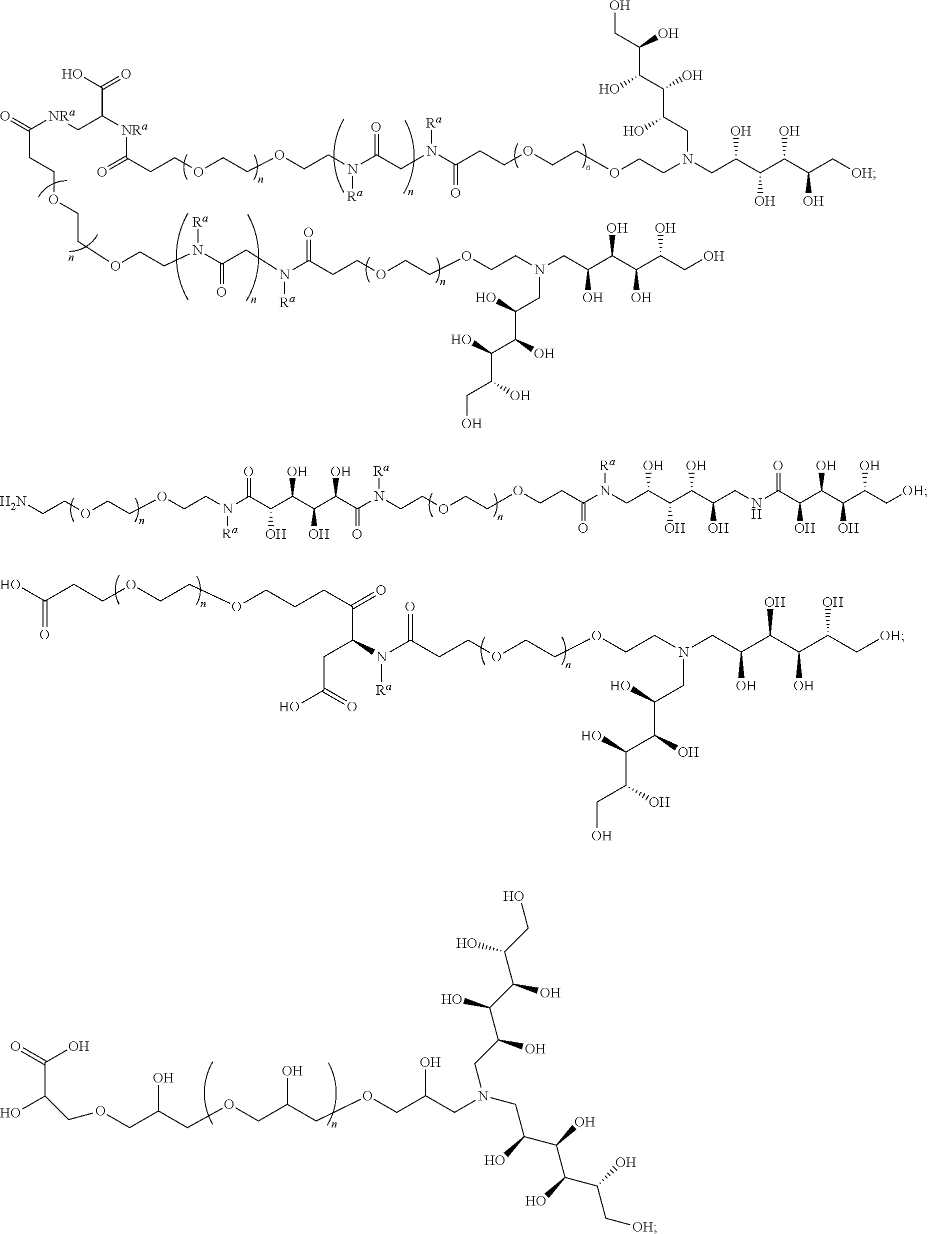

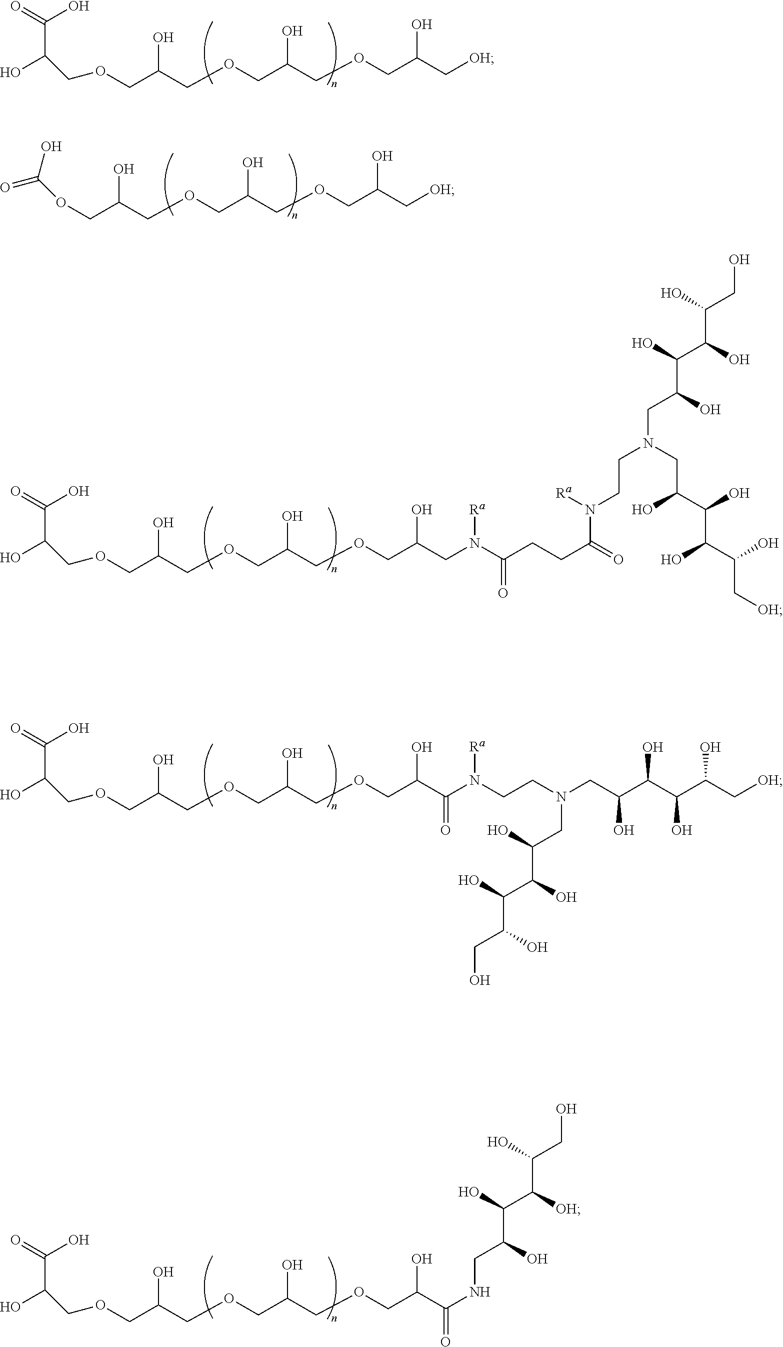

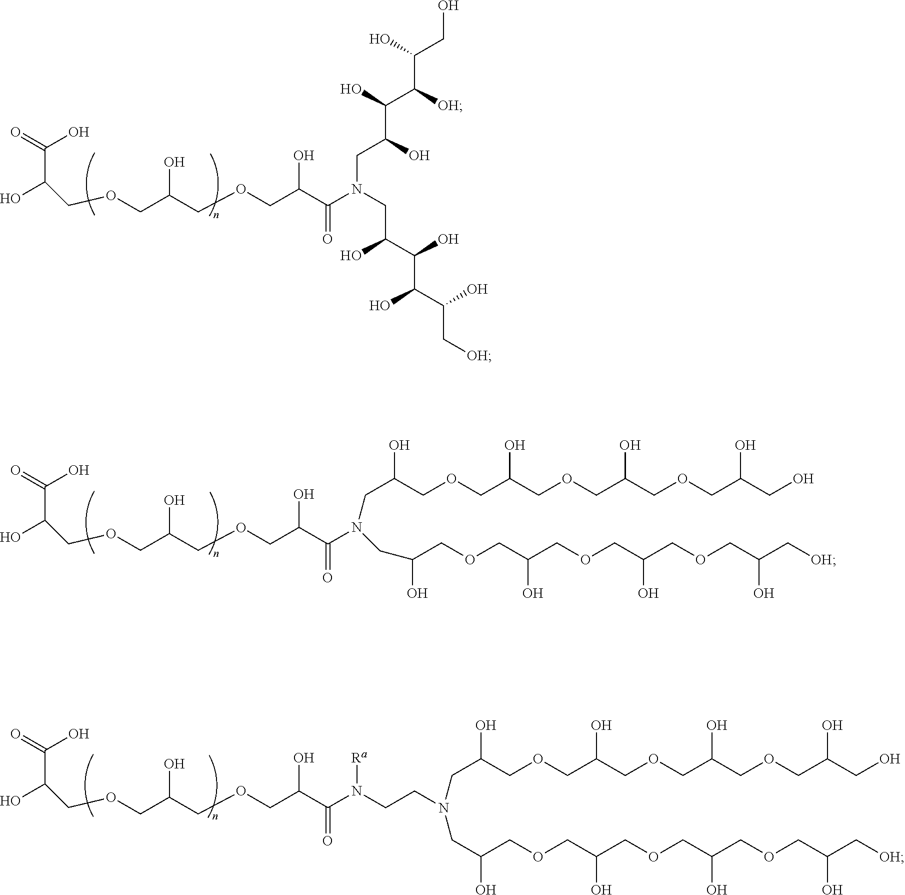

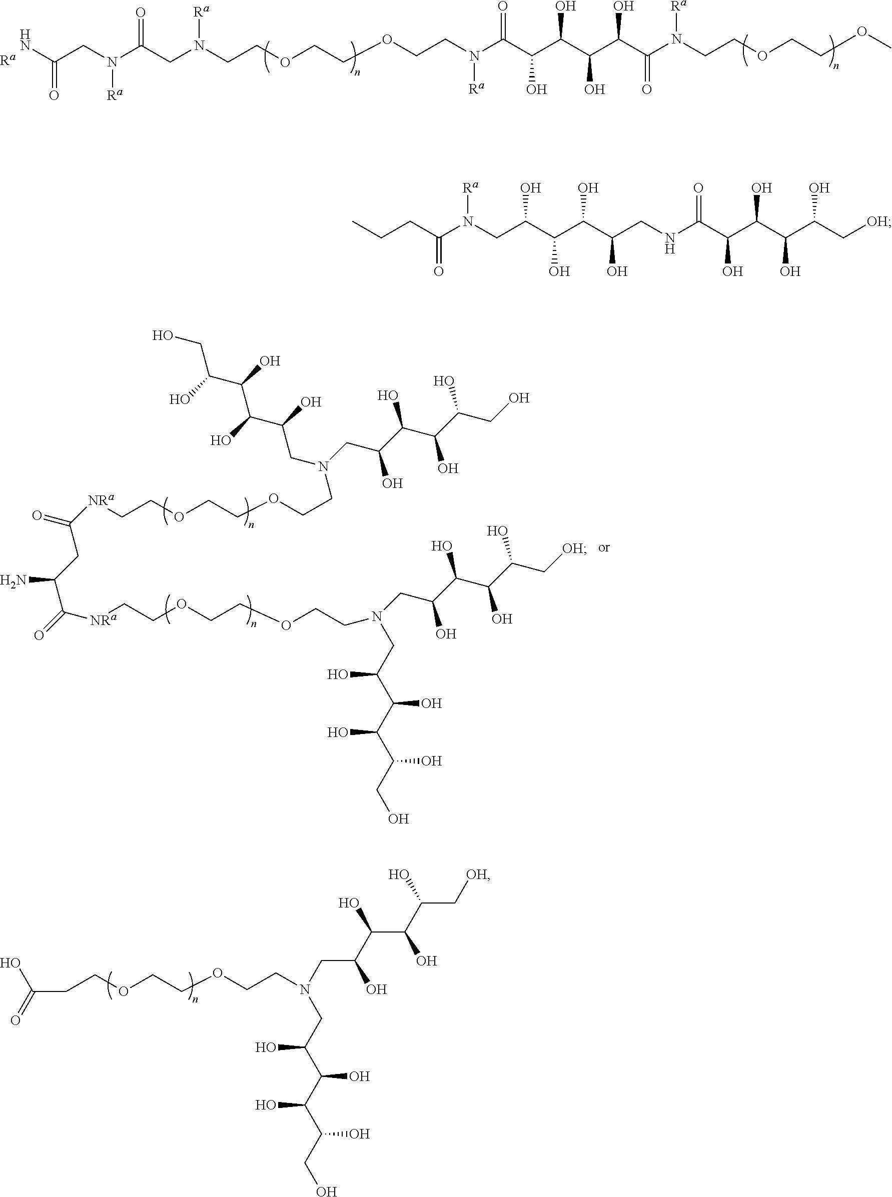

or a stereoisomer thereof, wherein R^{a} is H or C₁₋₆ alkyl and n is 1-20.

In some embodiments, provided is a Linker compound, wherein the Polar group has one of the following structures prior to attachment to the Amino Acid unit:

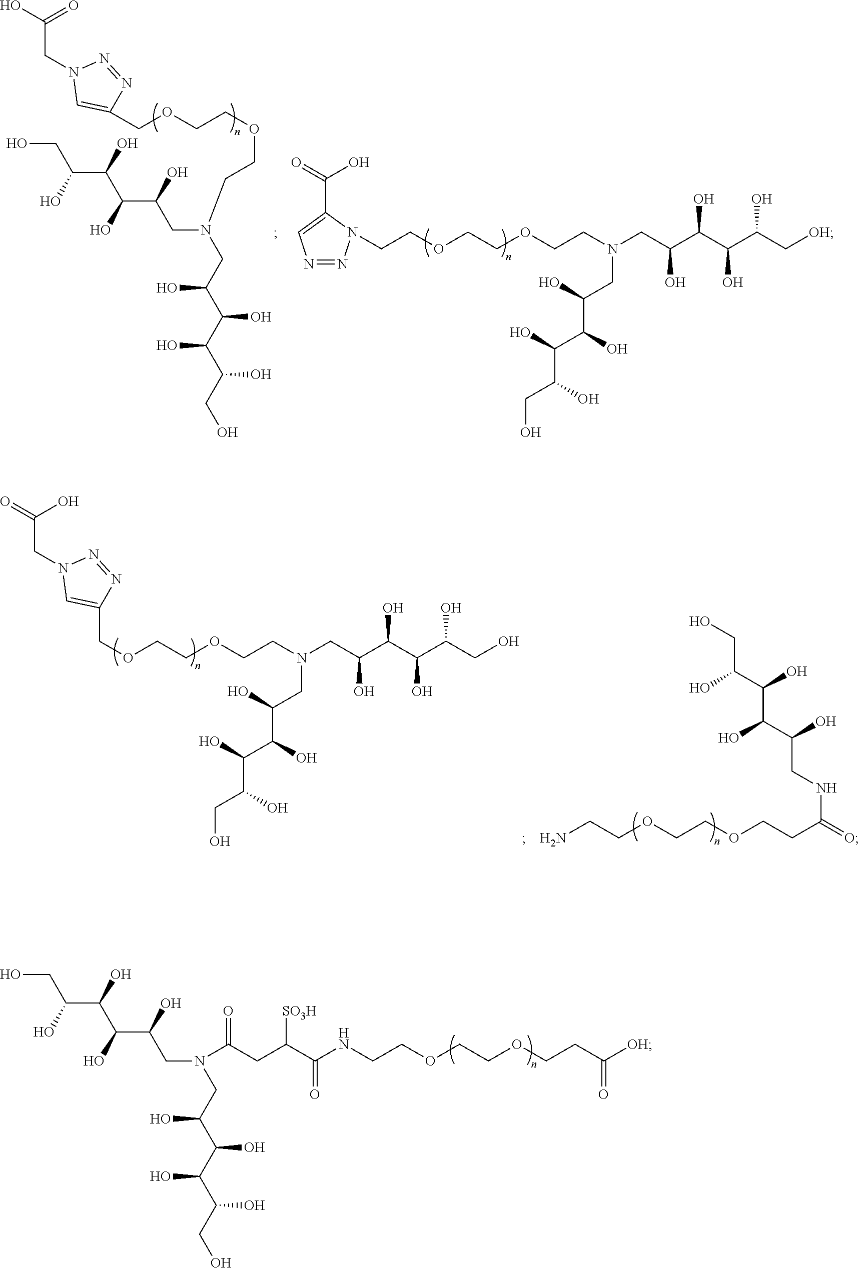

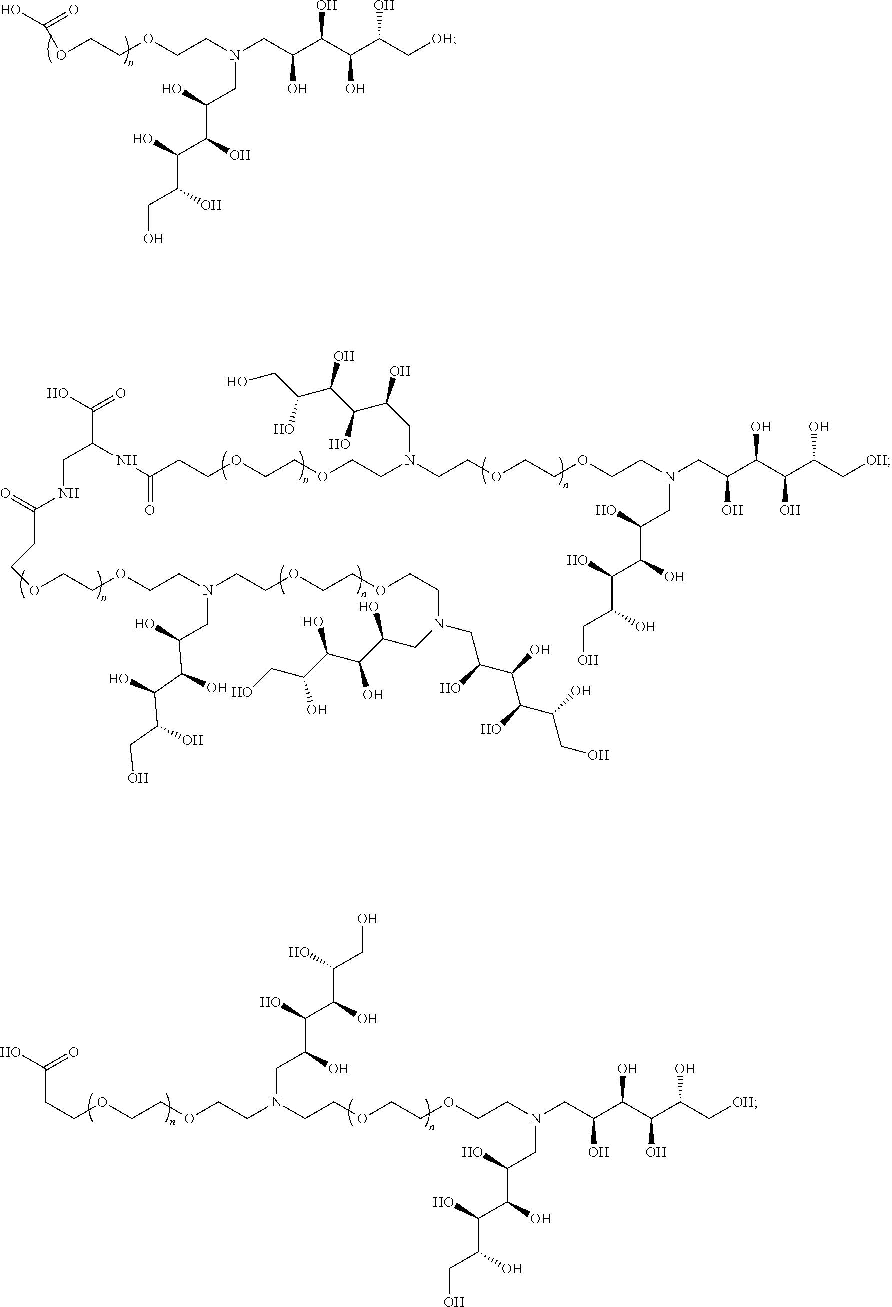

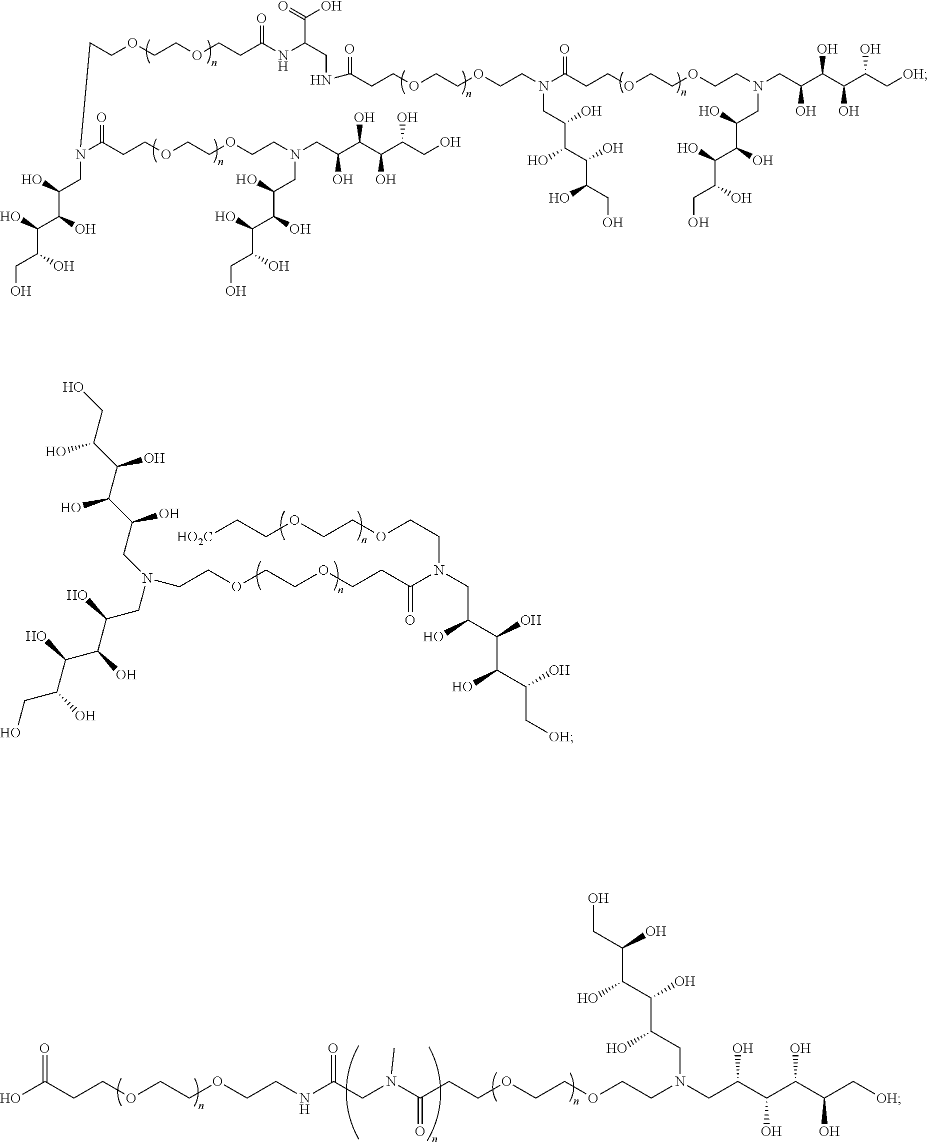

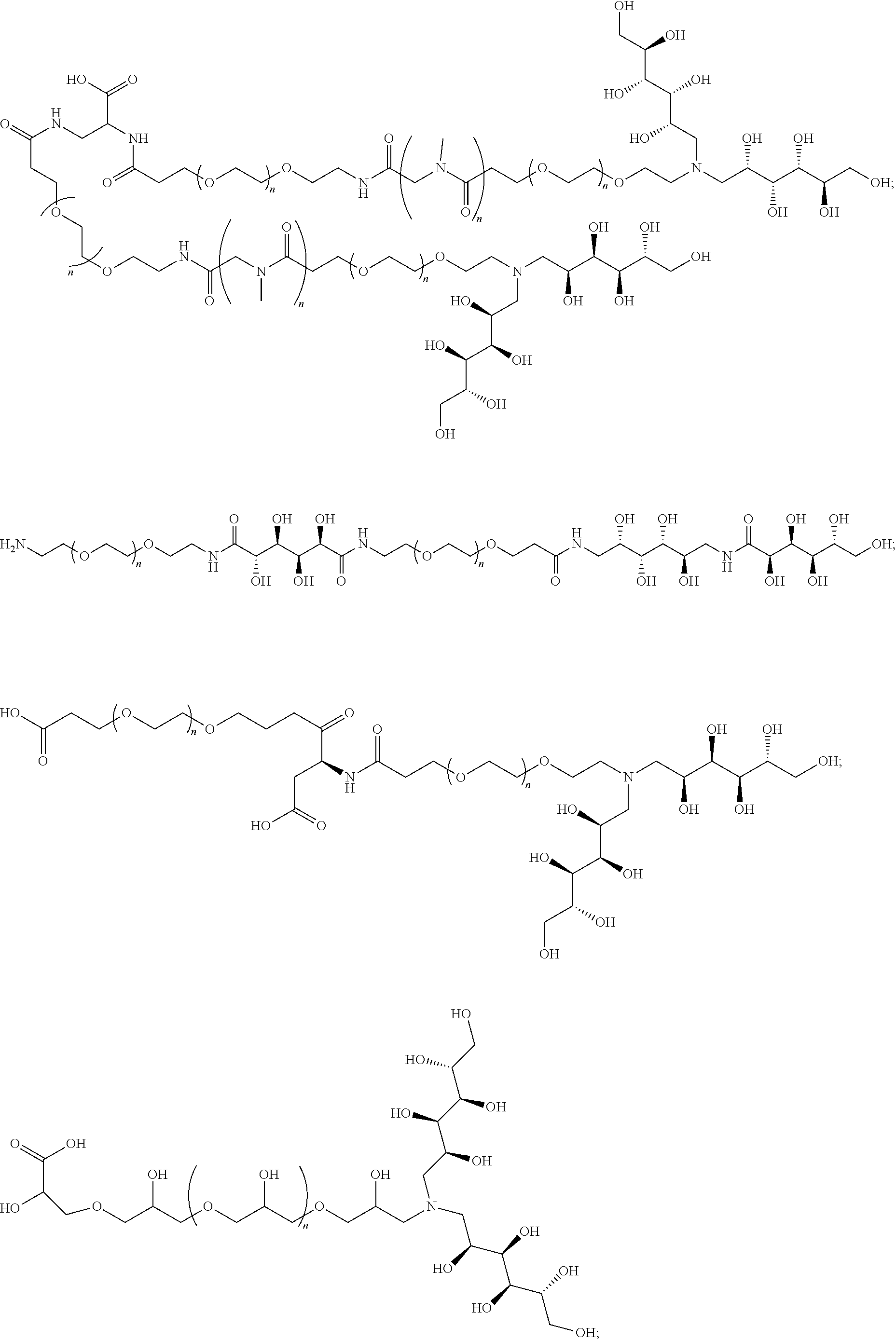

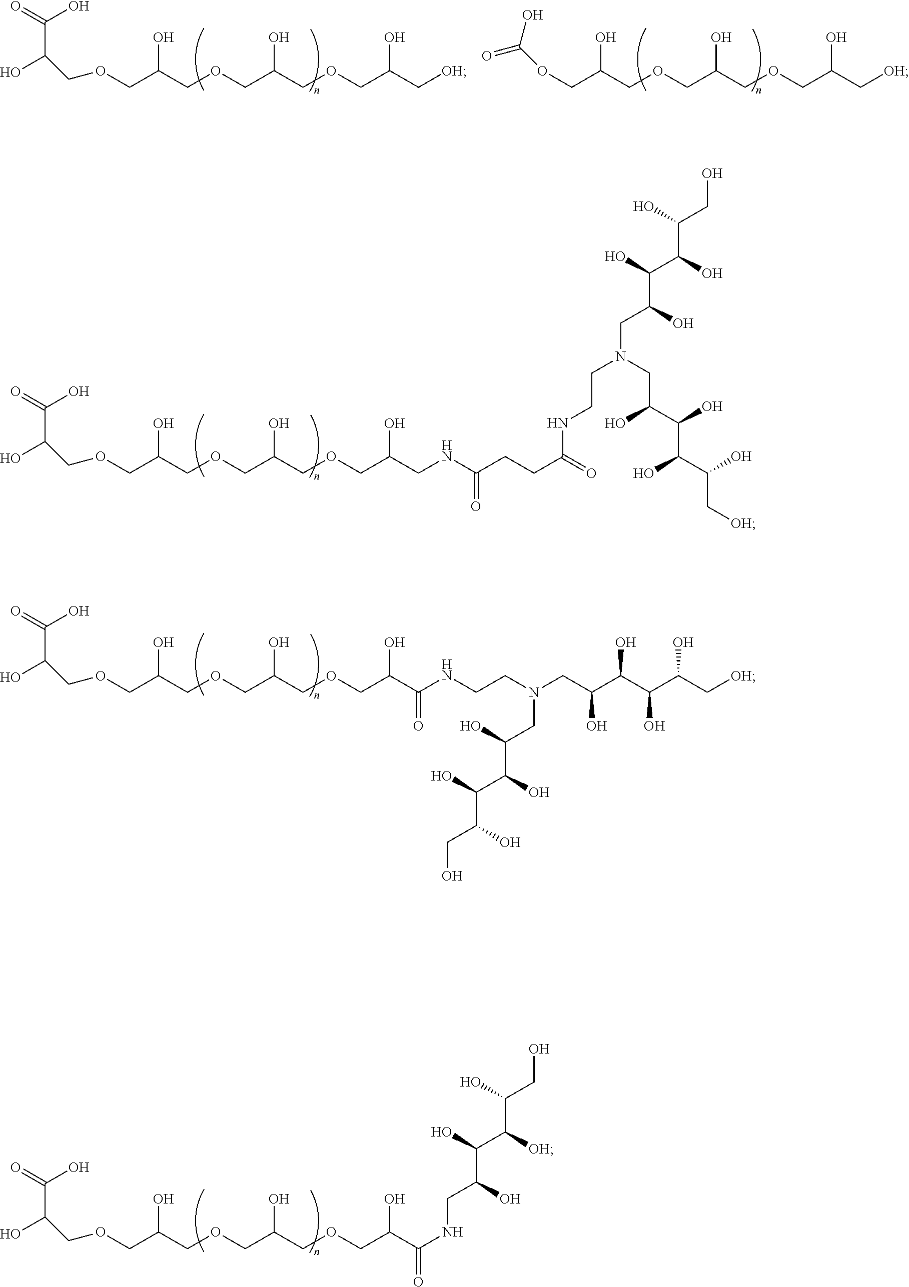

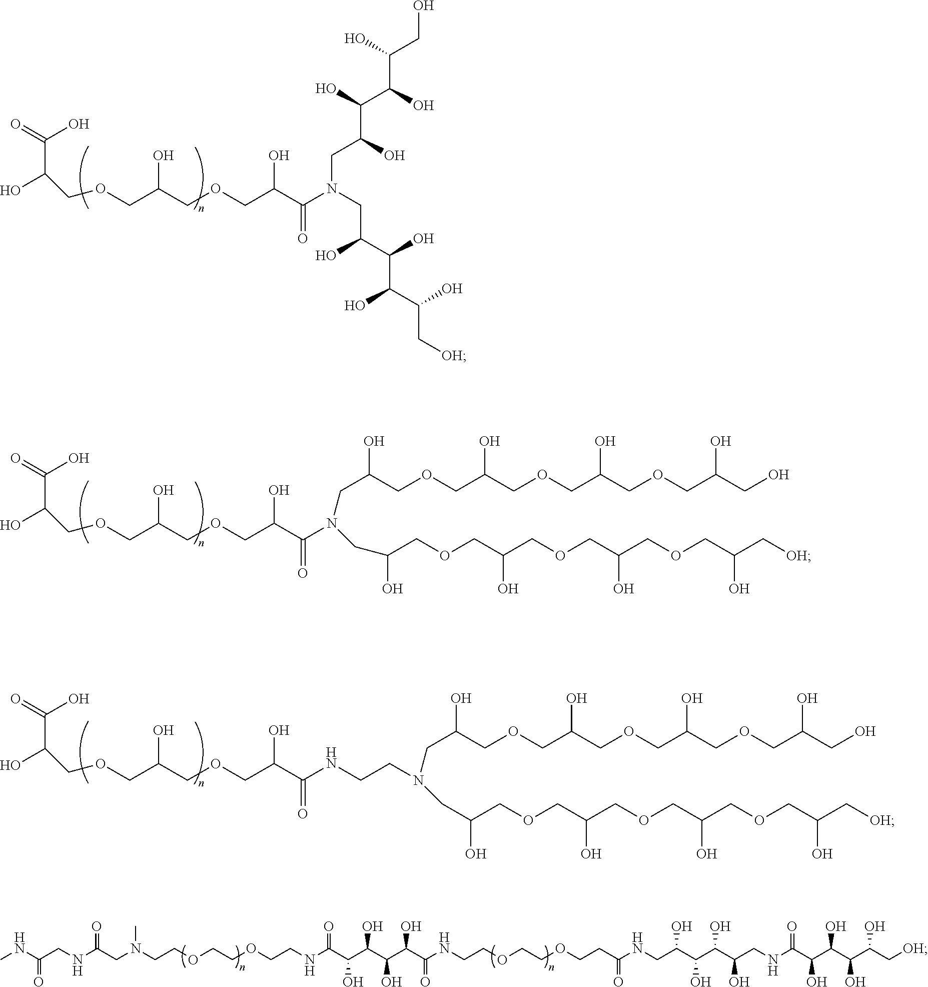

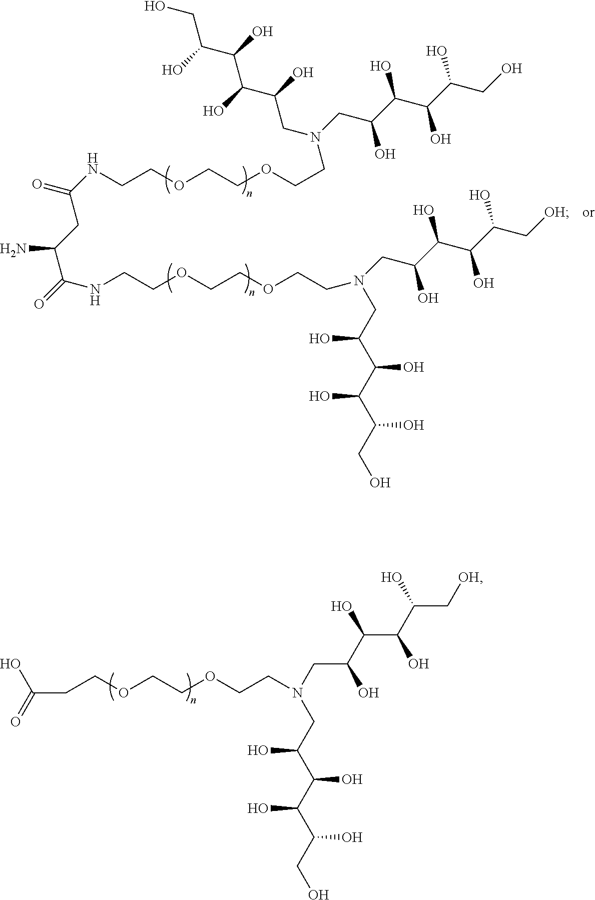

or a stereoisomer thereof, wherein R^{a} is H or C₁₋₆ alkyl and n is 1-20.

In some embodiments, provided is a Linker compound, comprising a Polar group selected from the following:

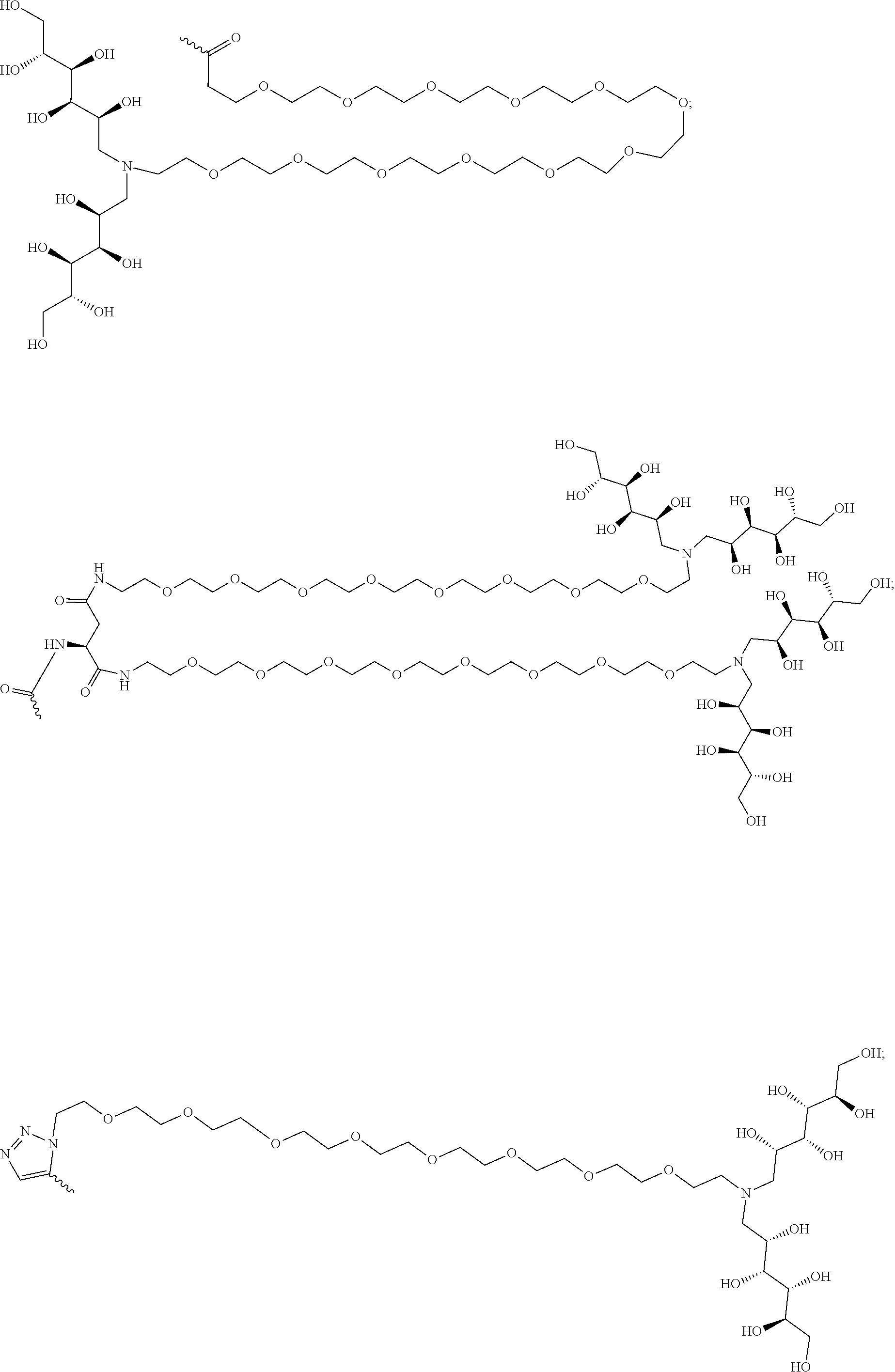

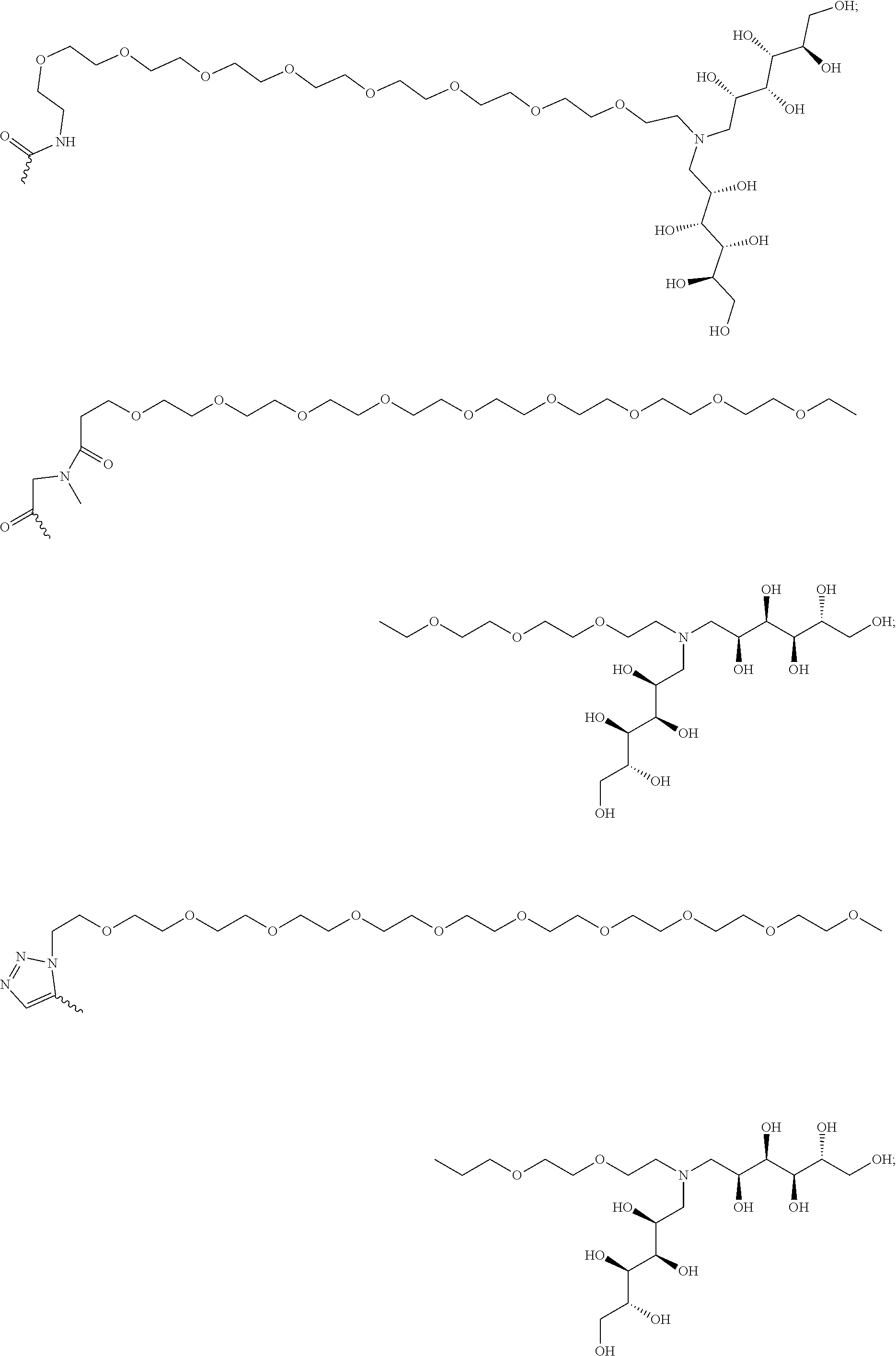

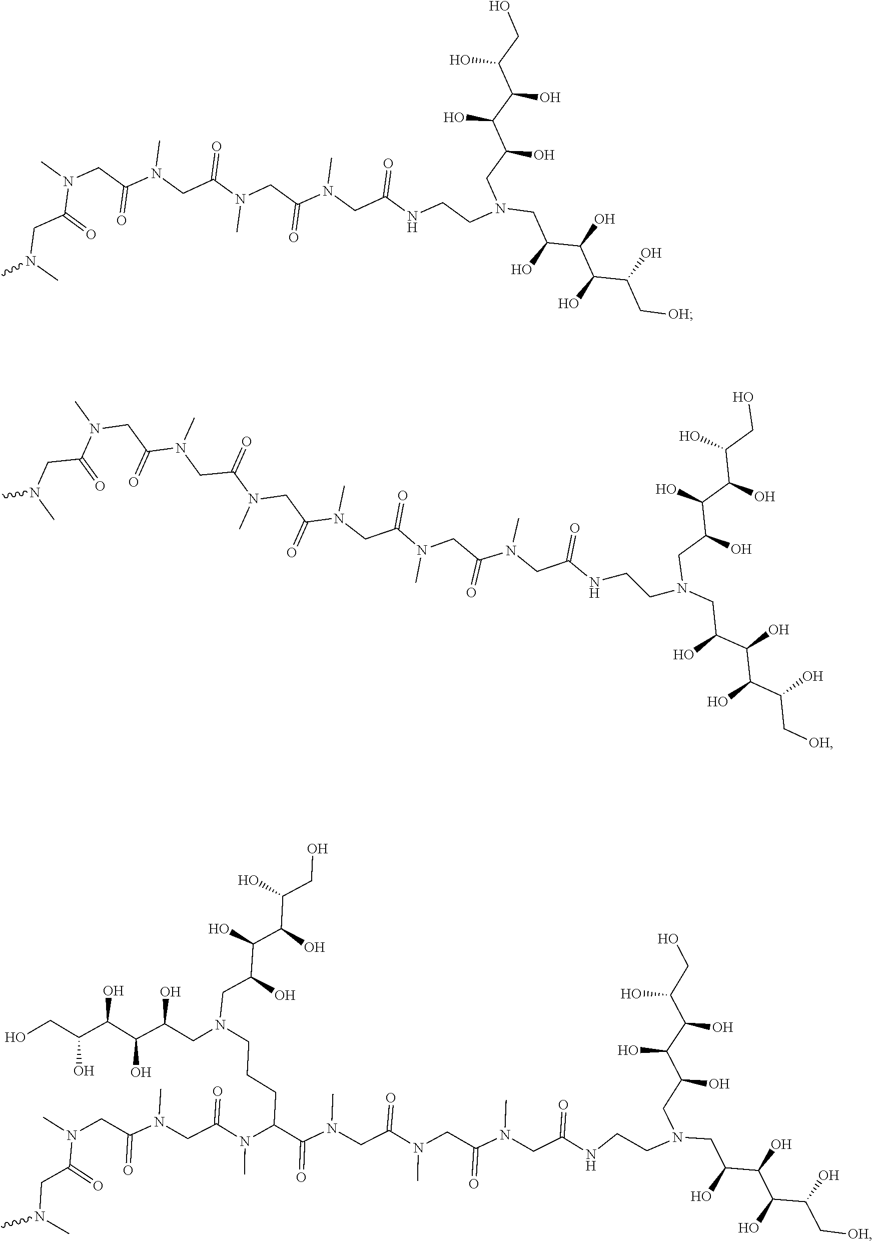

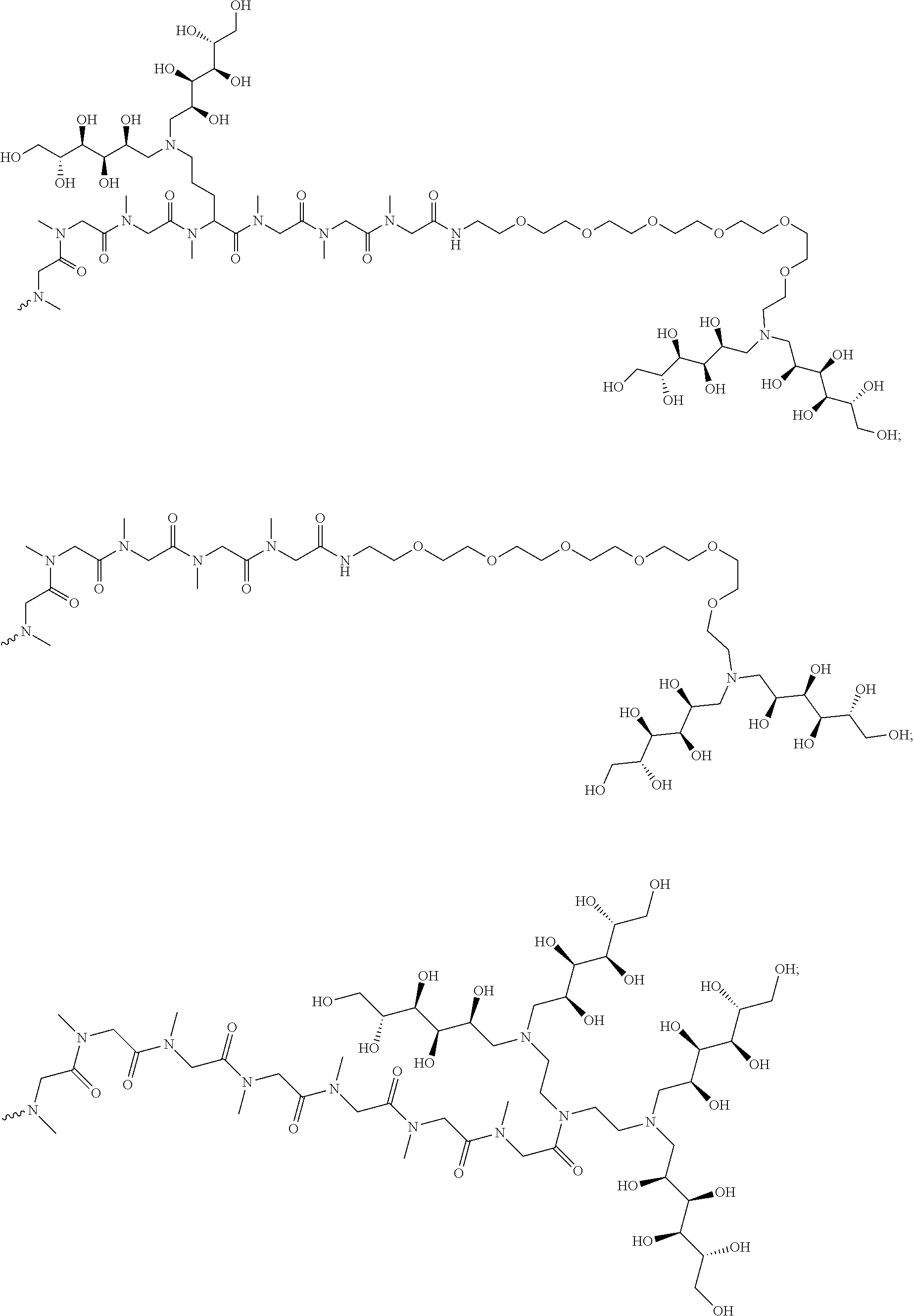

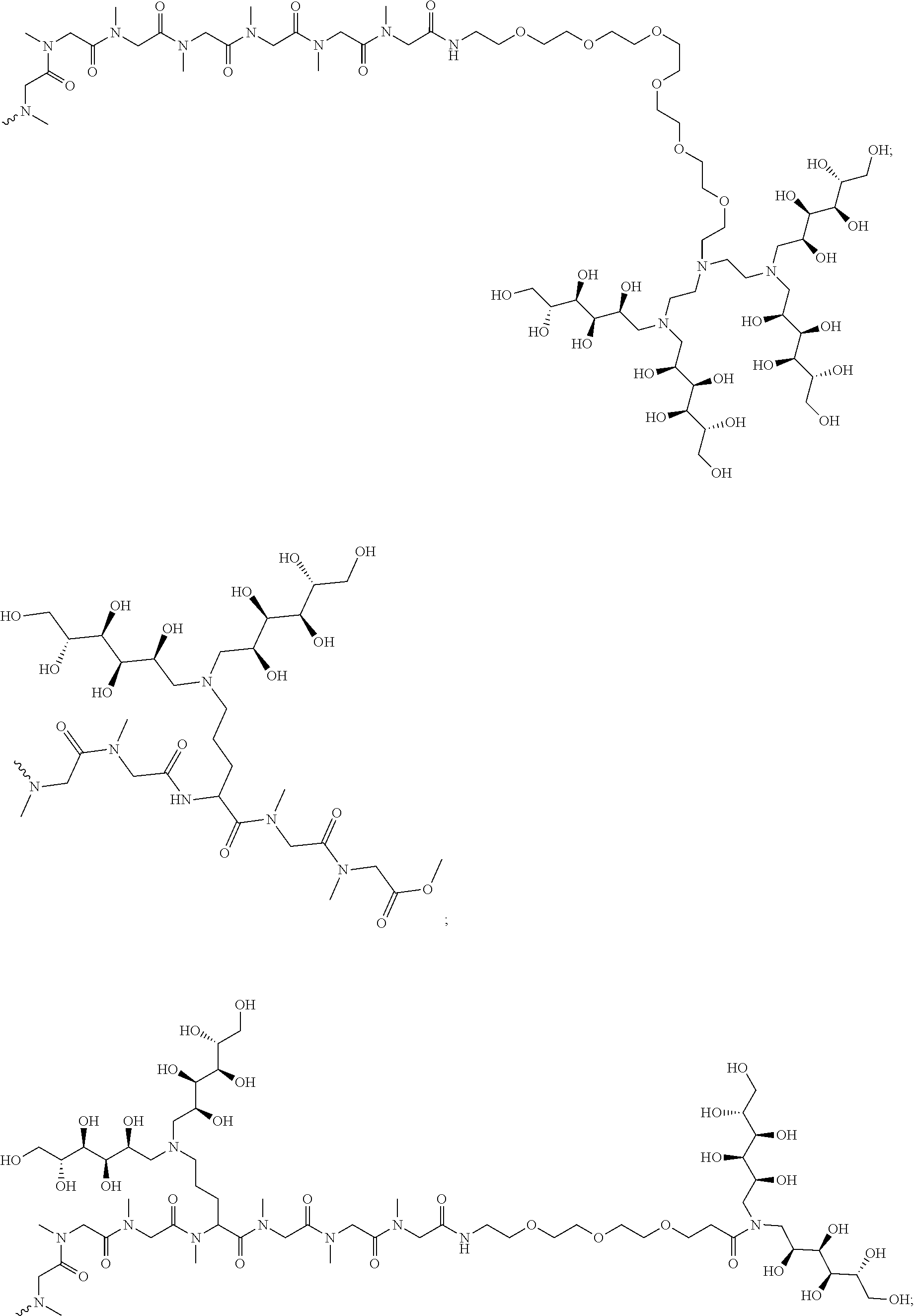

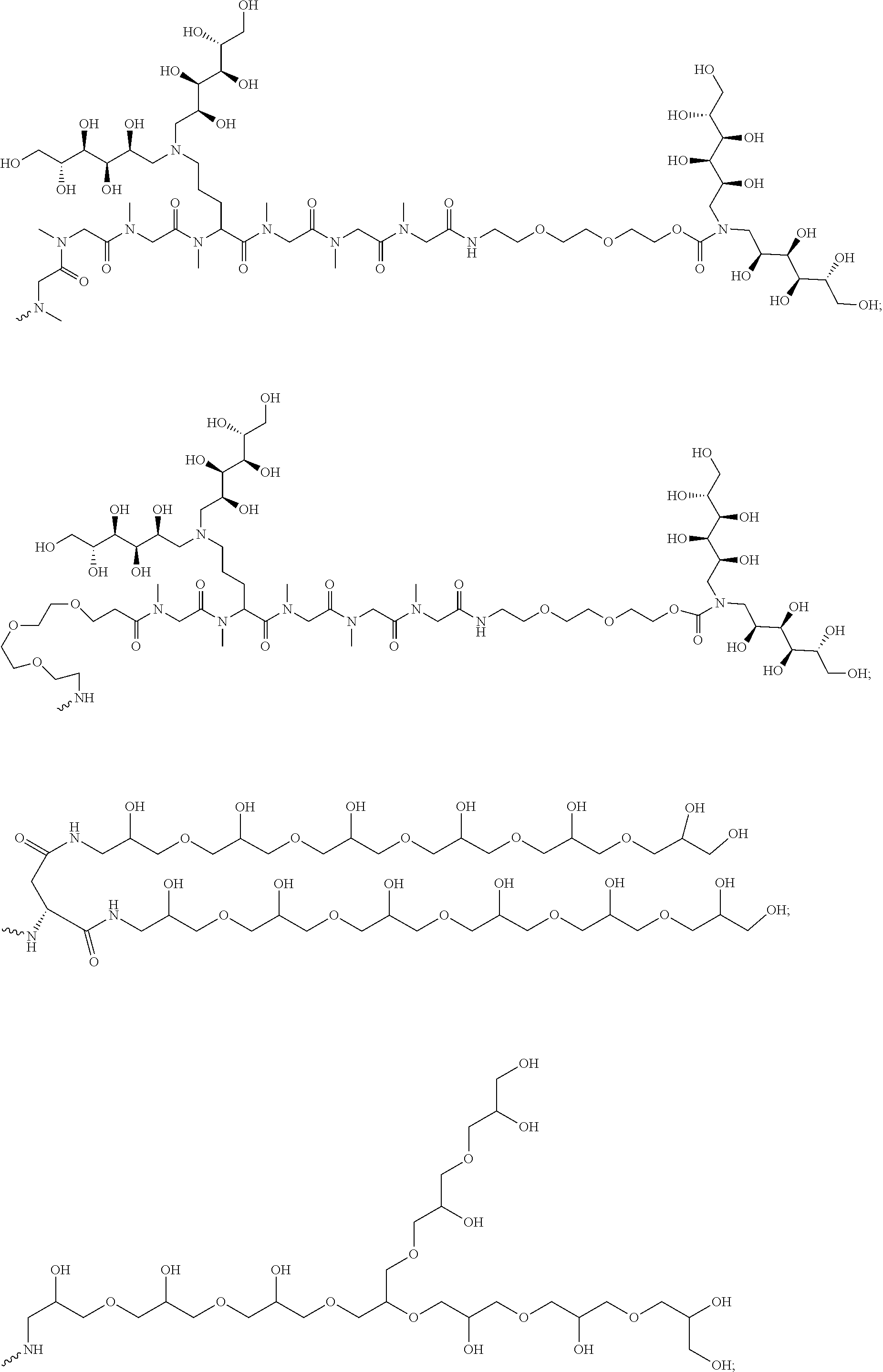

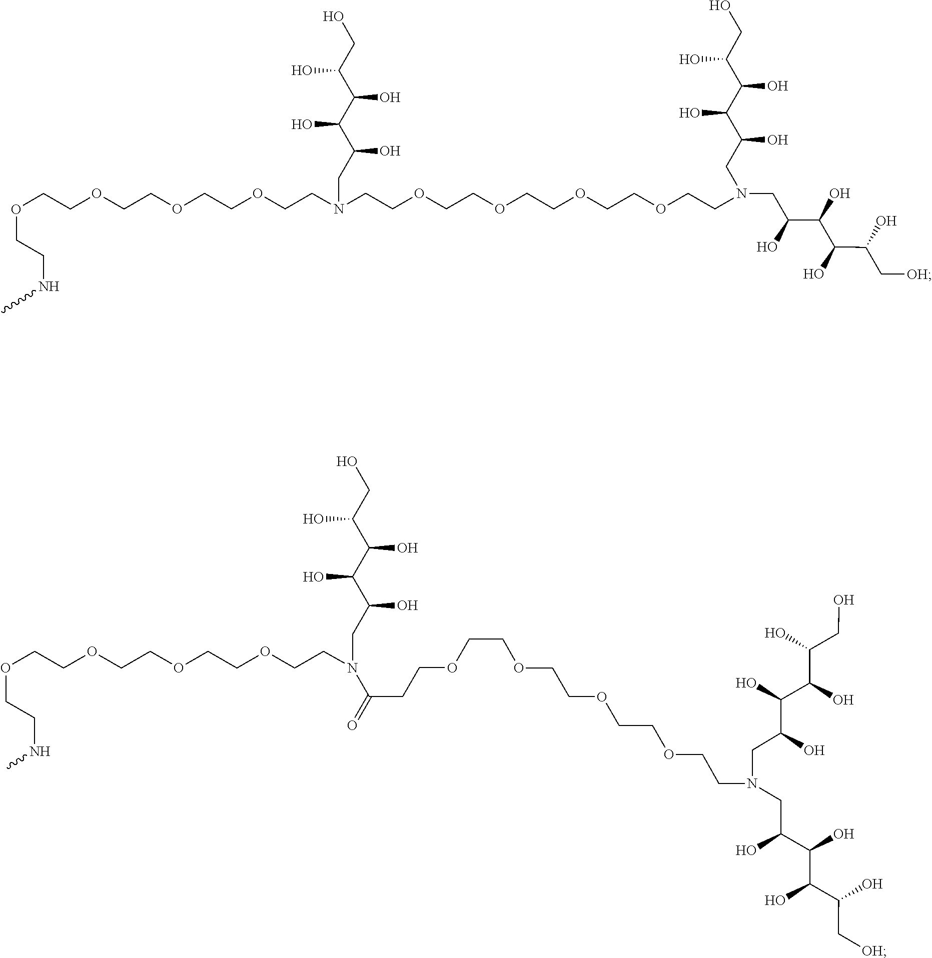

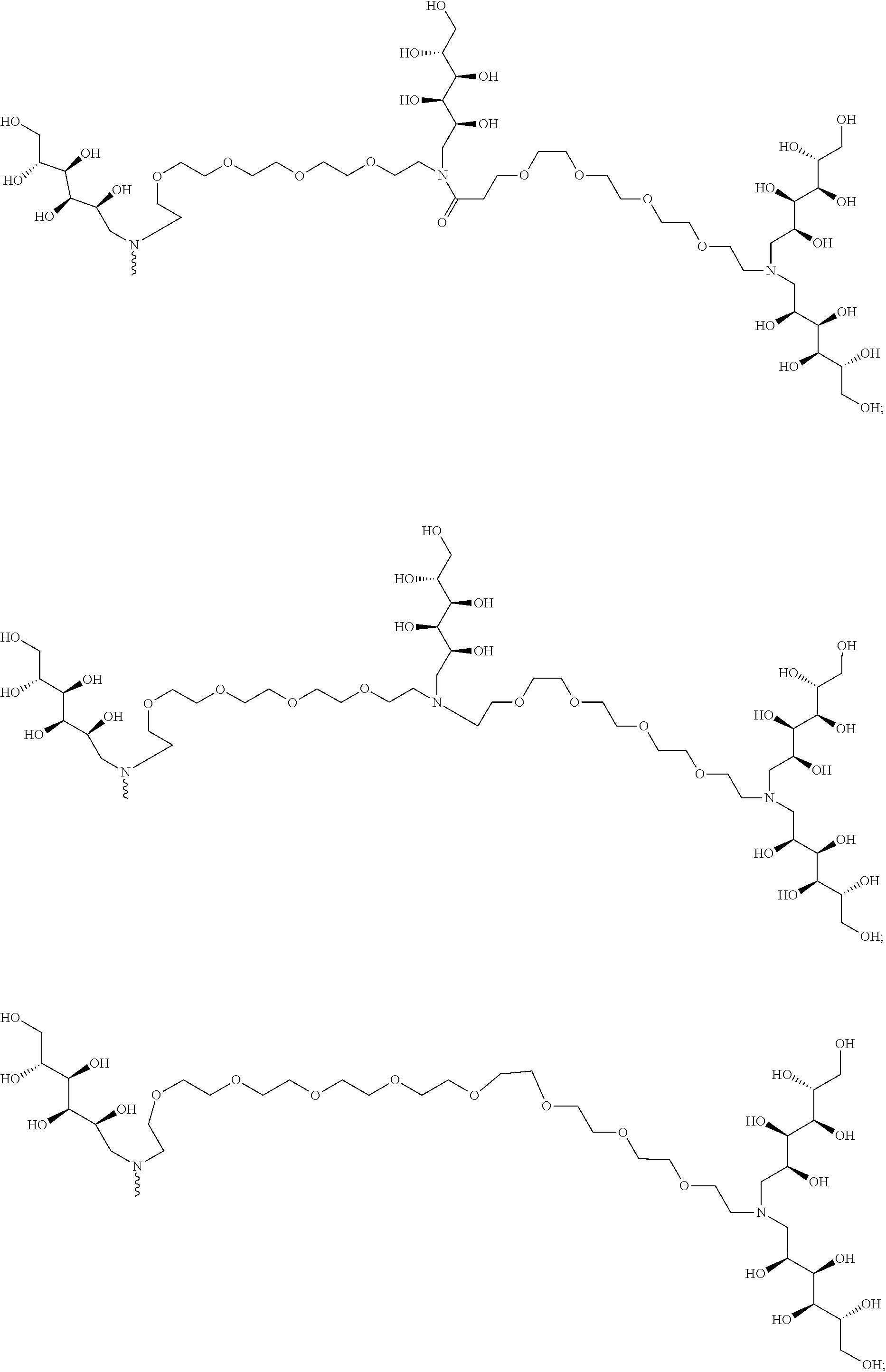

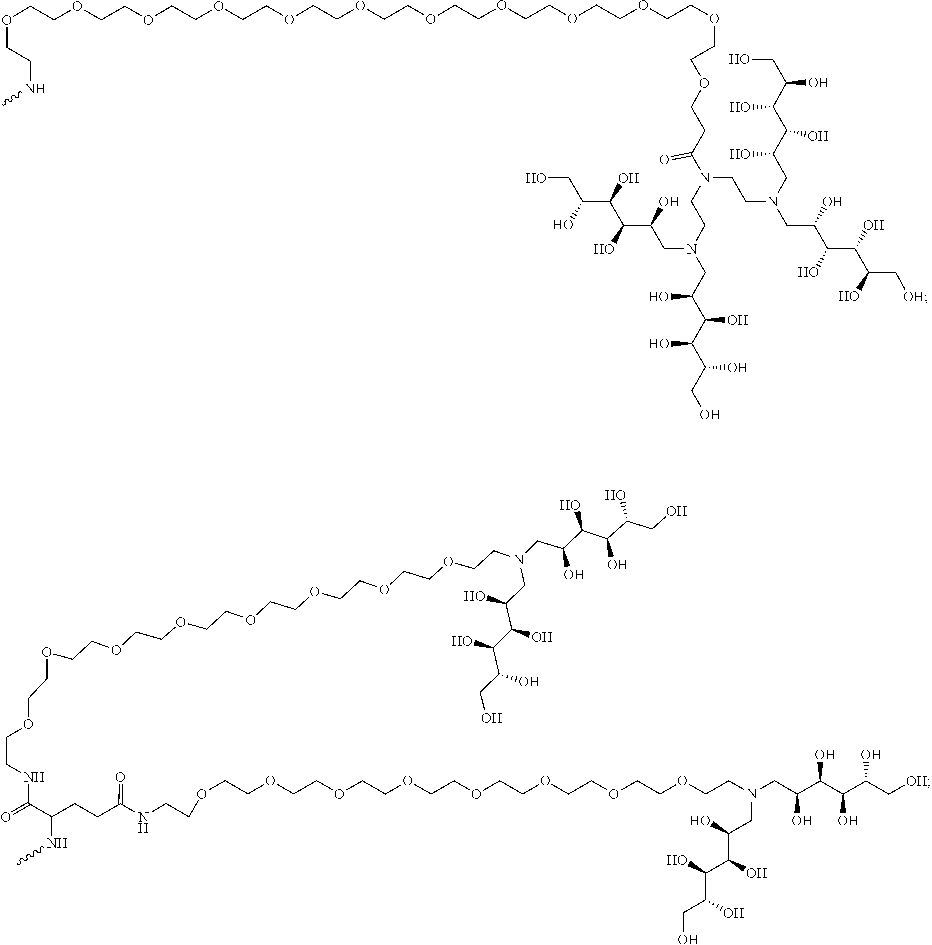

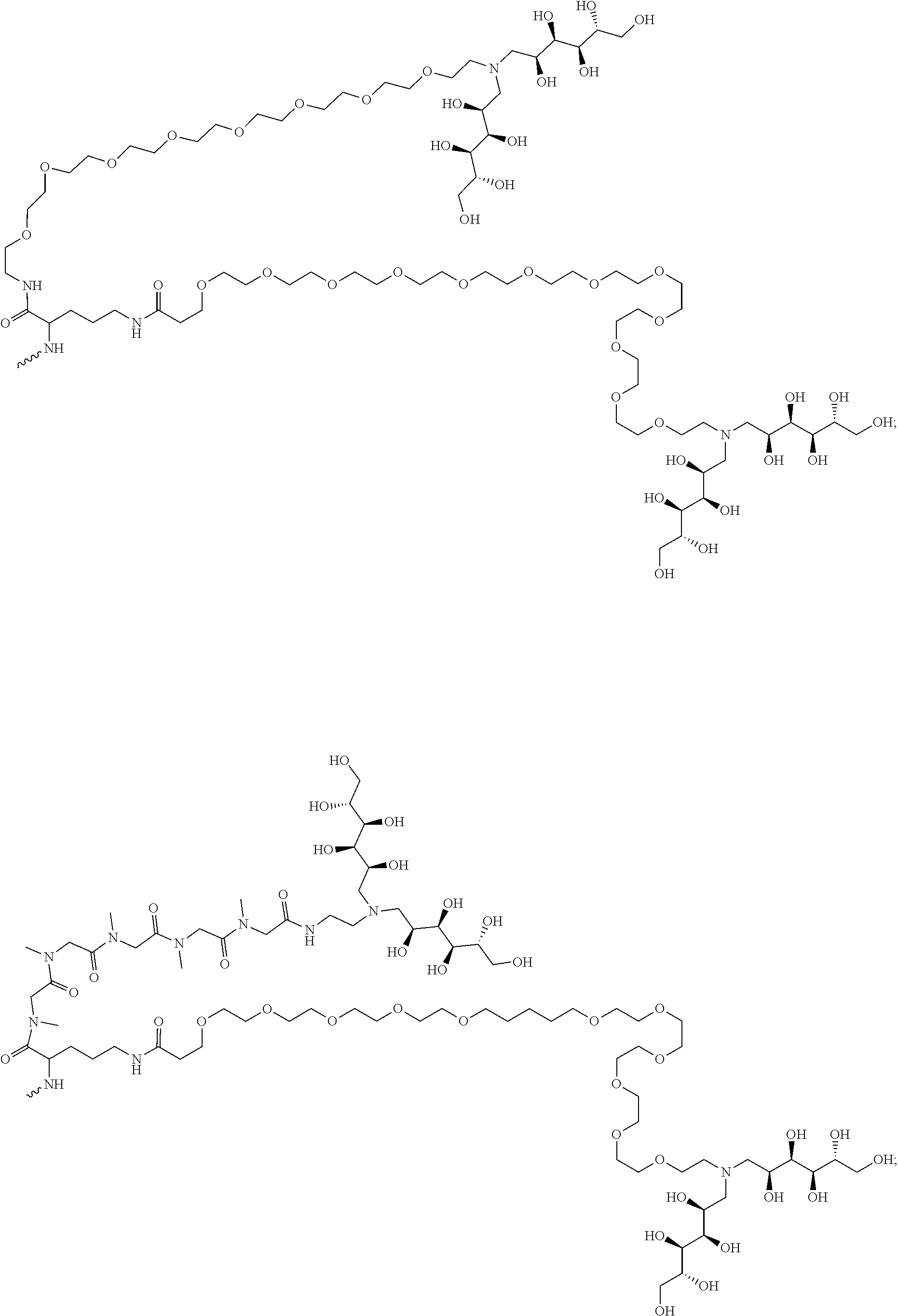

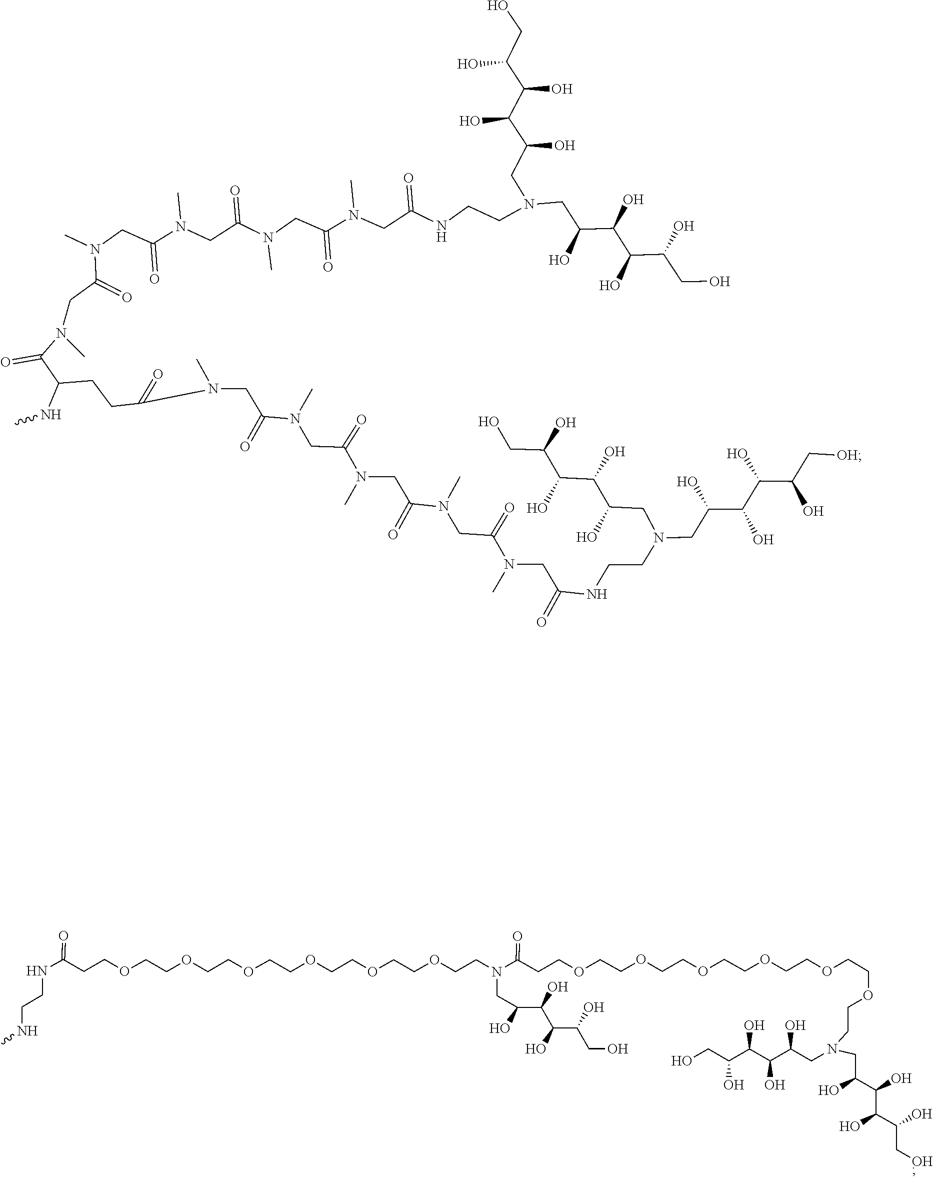

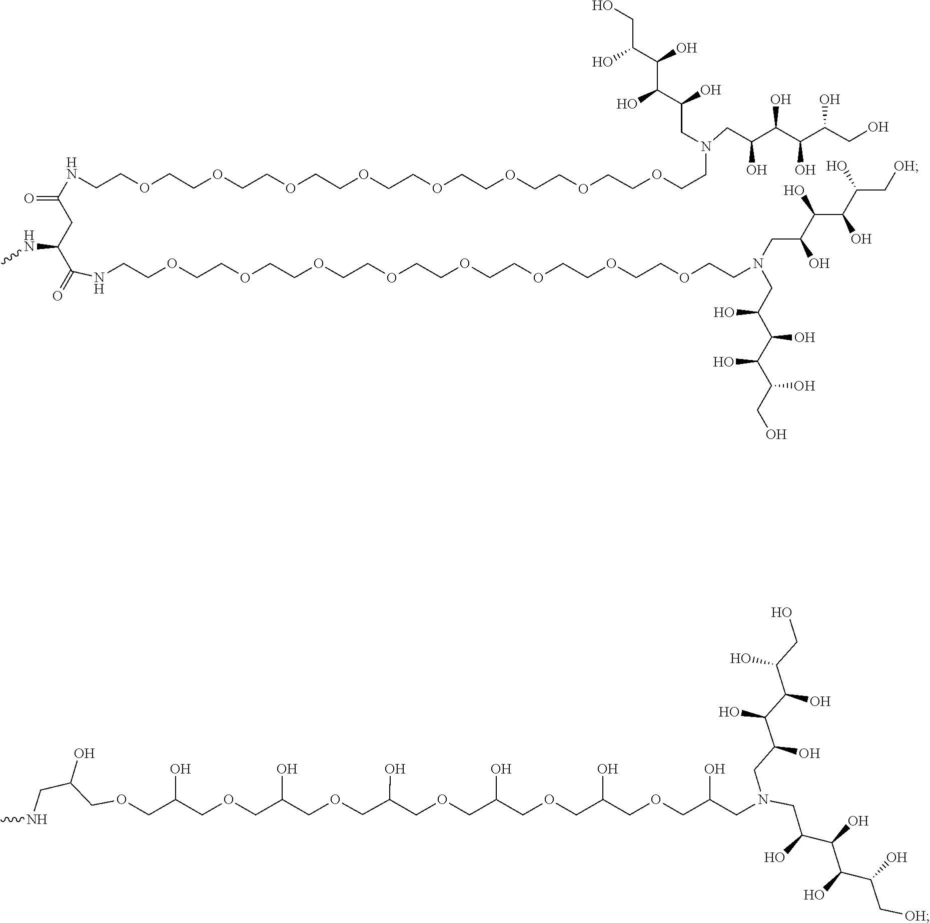

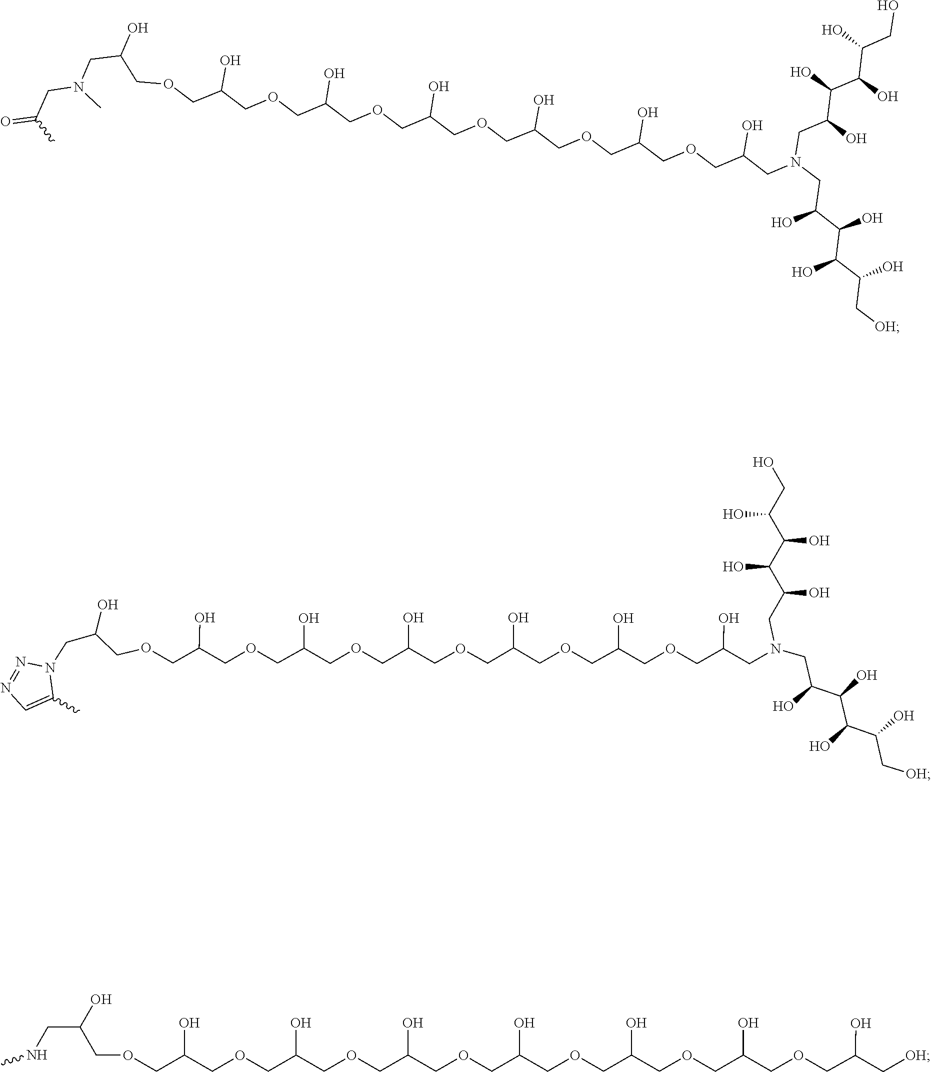

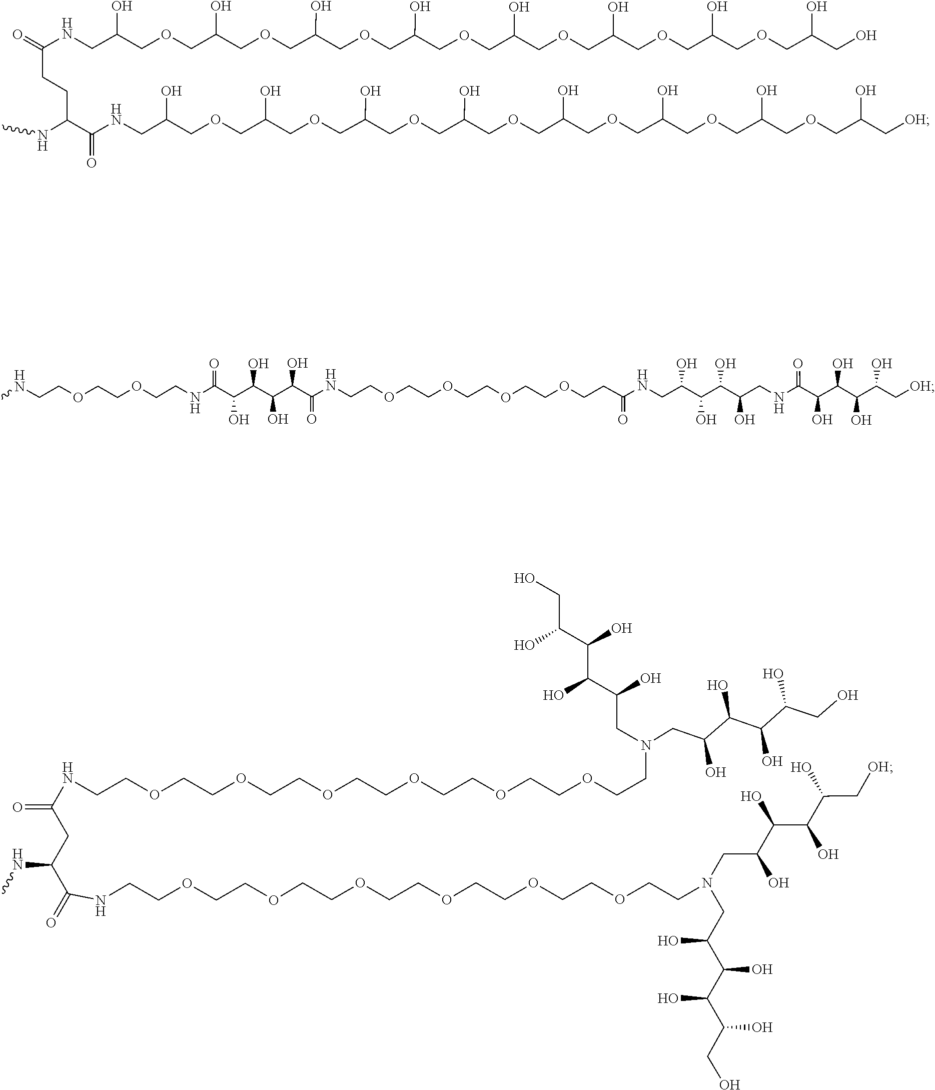

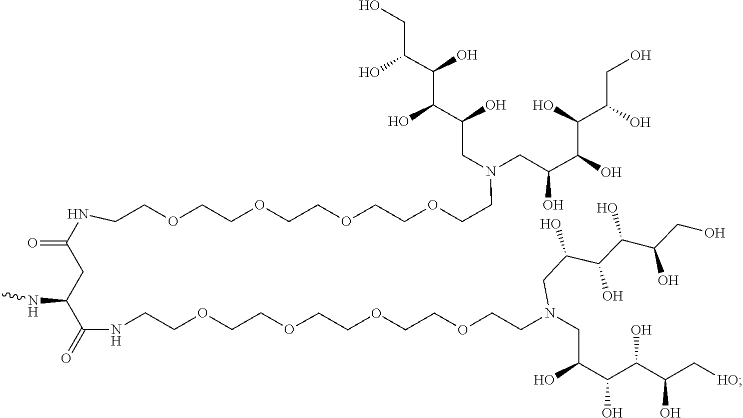

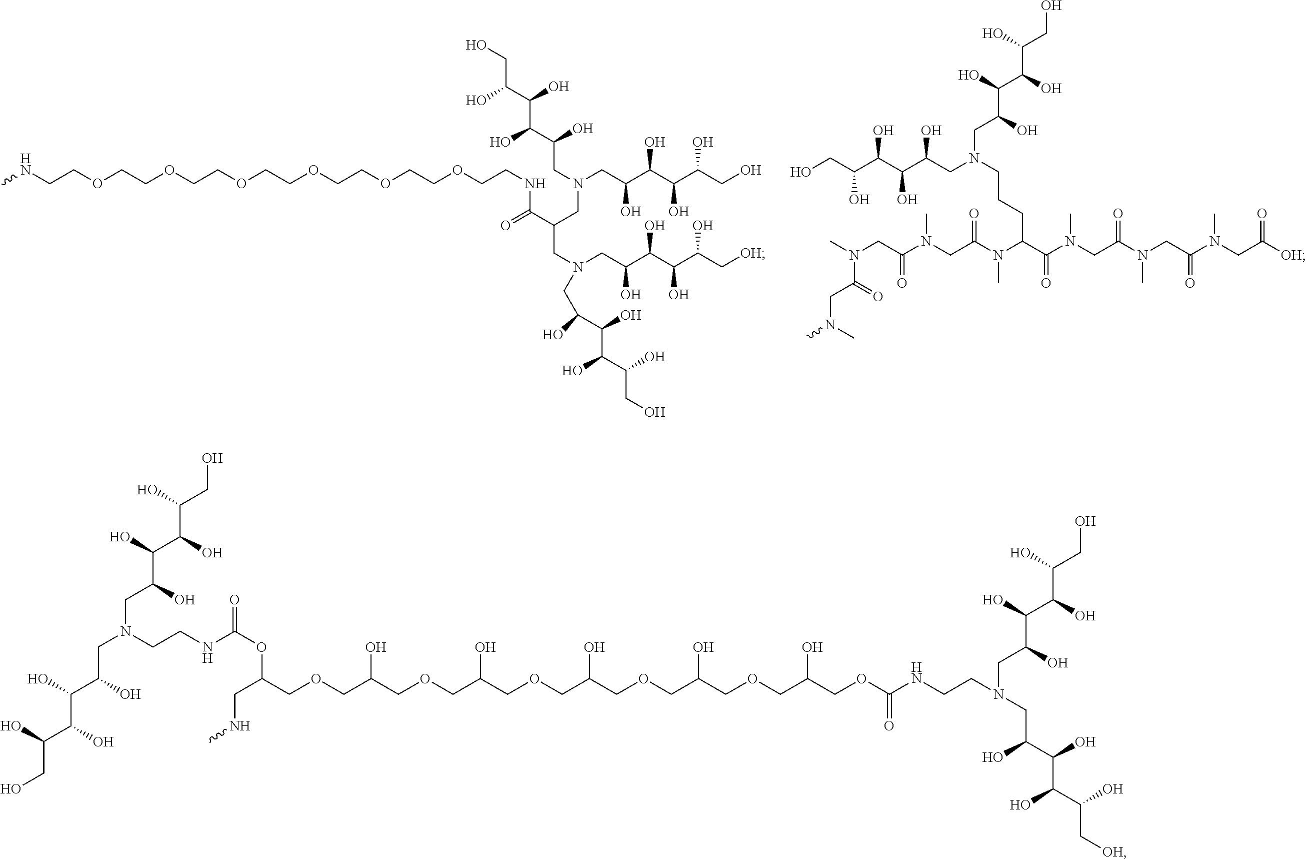

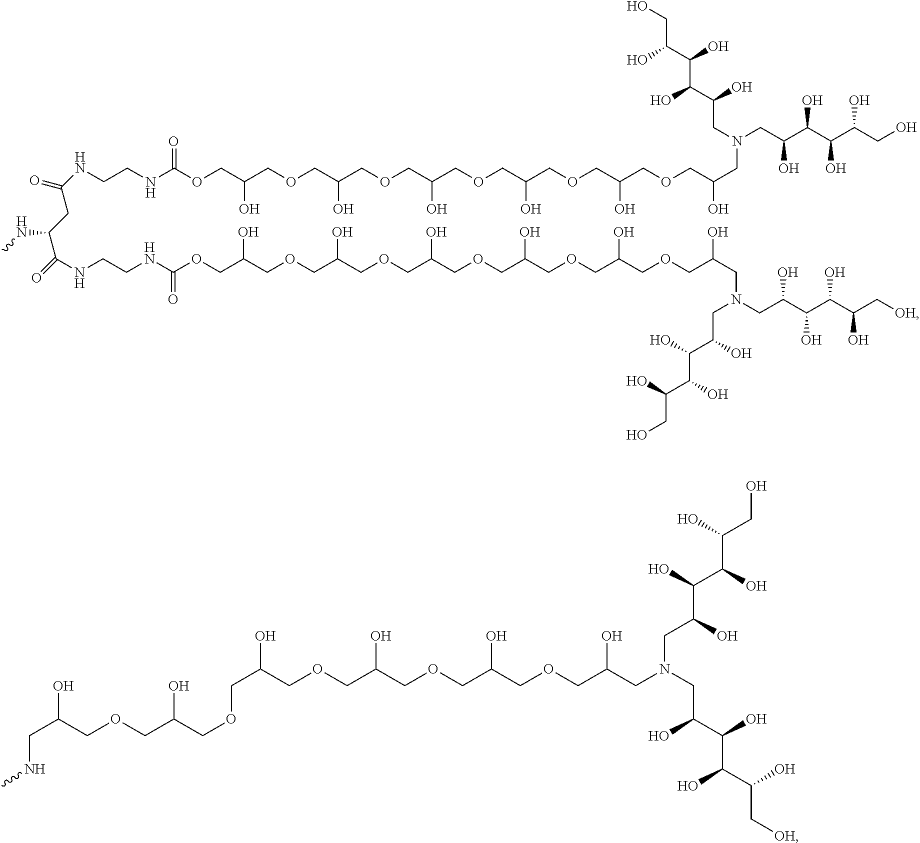

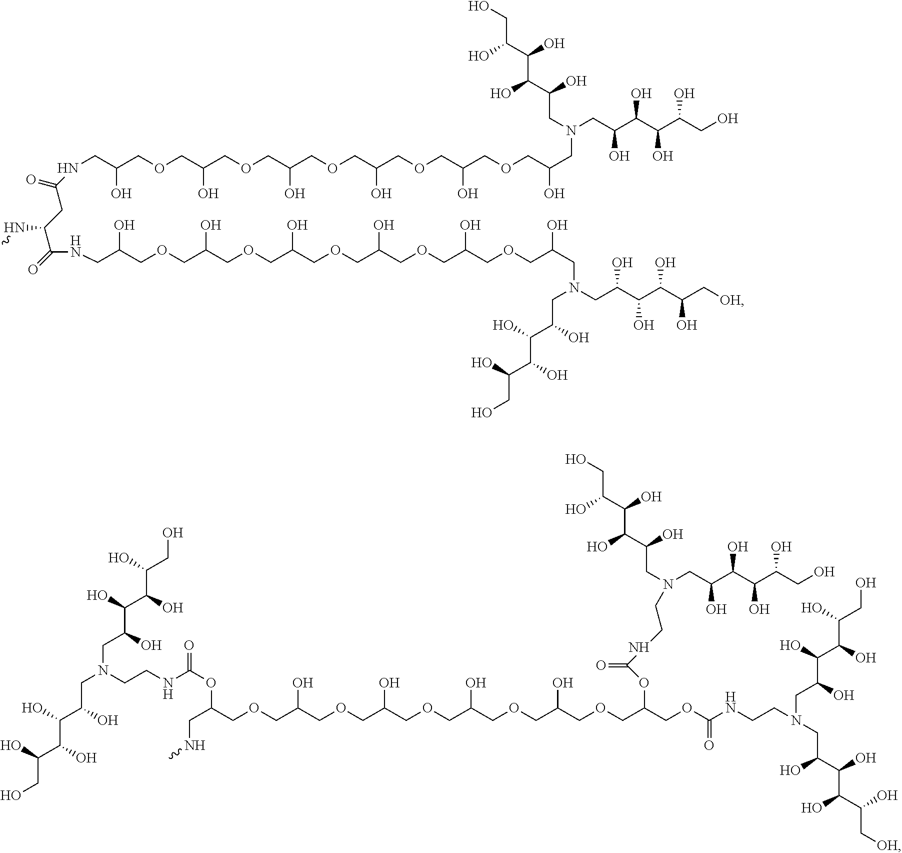

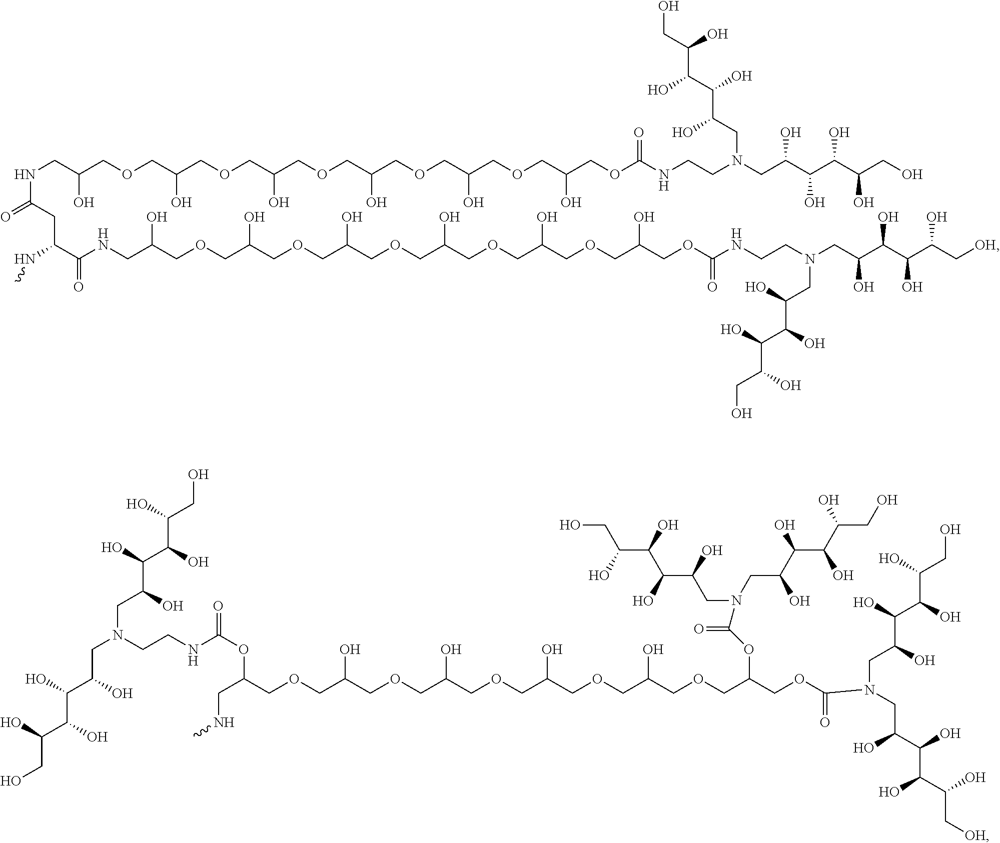

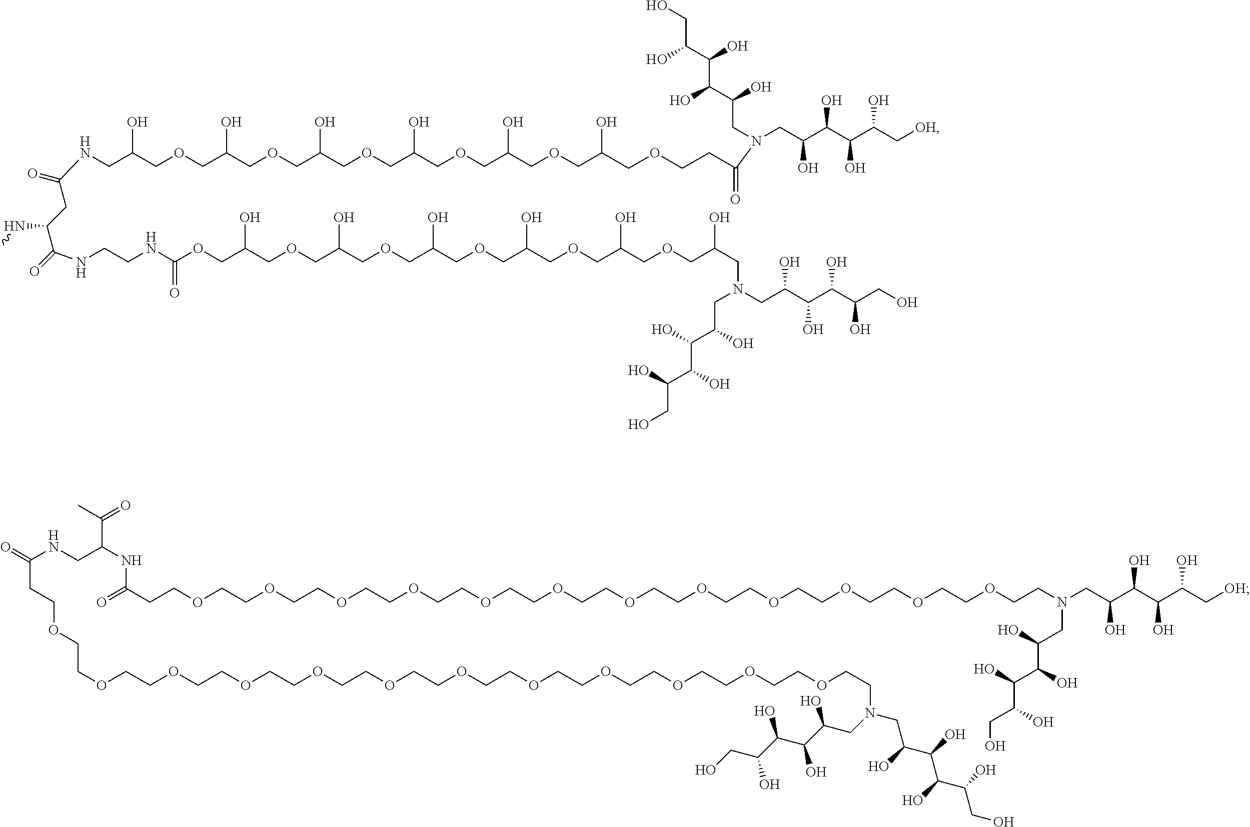

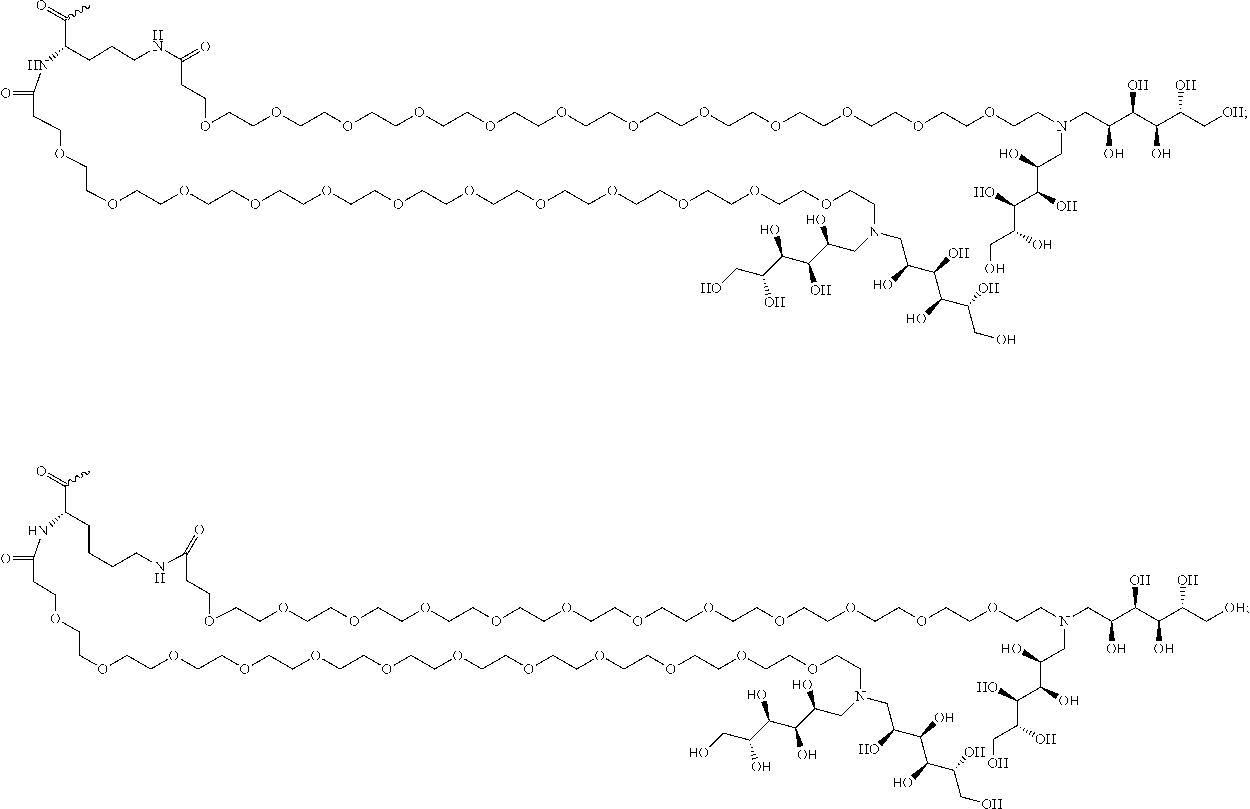

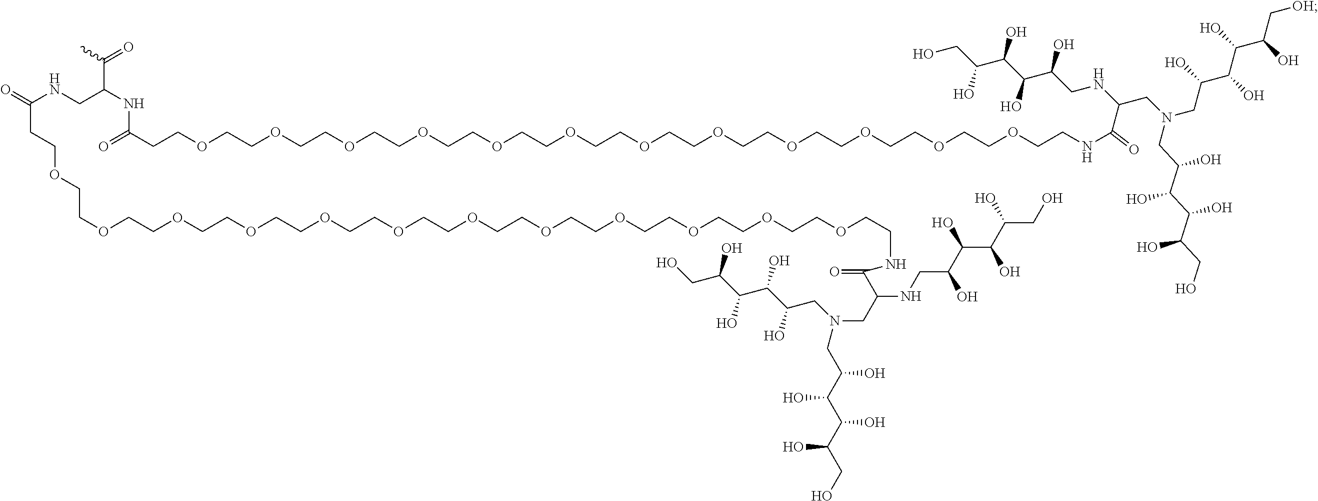

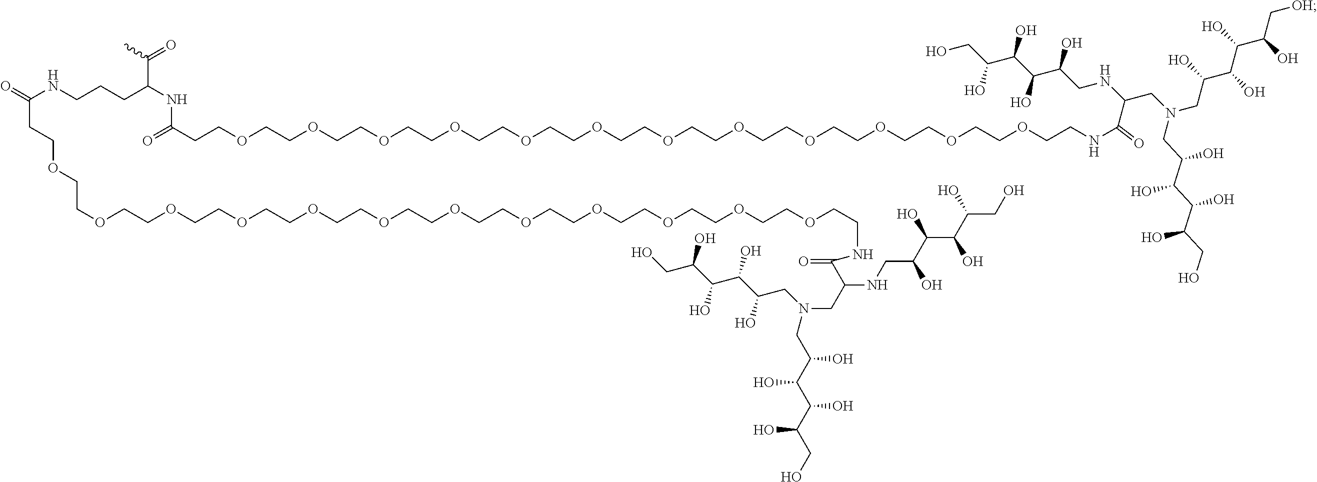

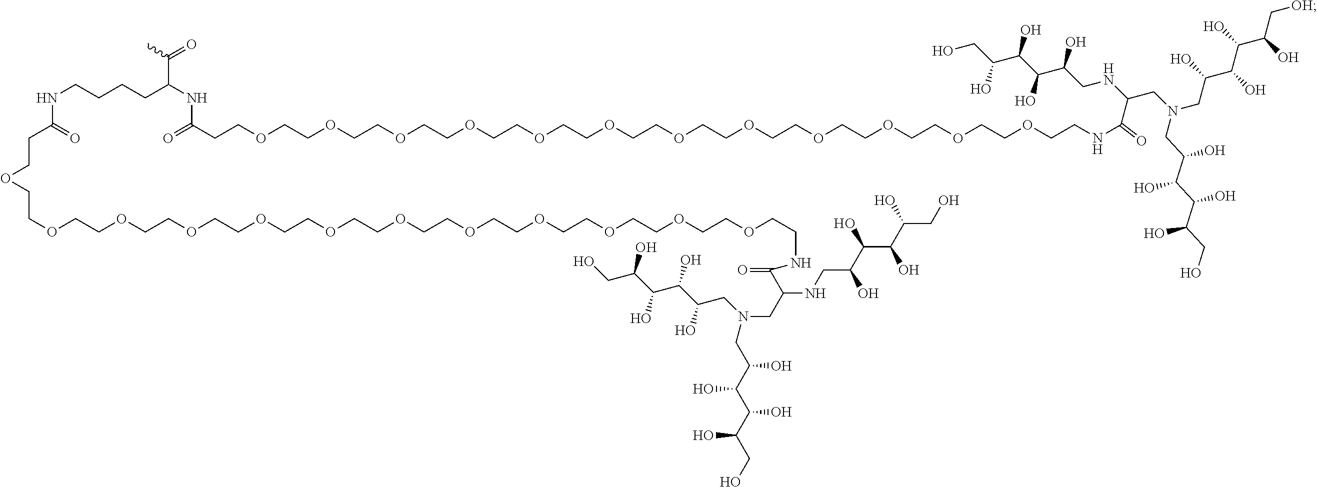

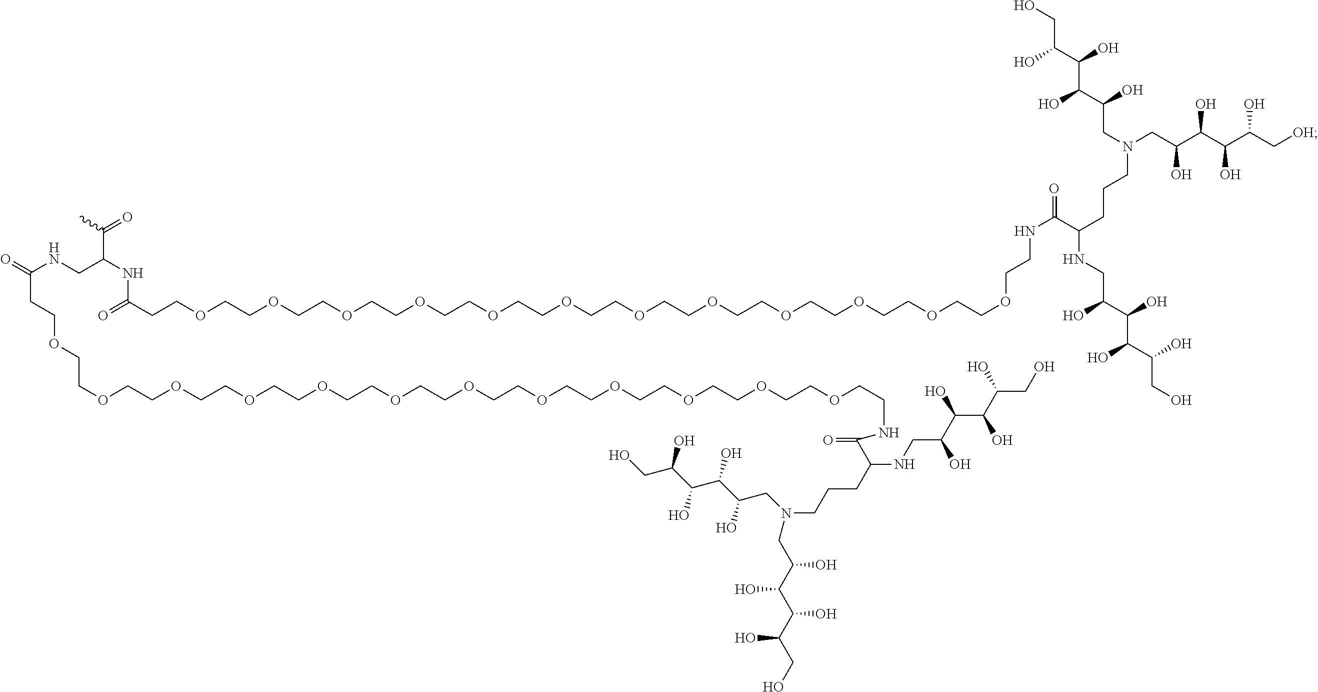

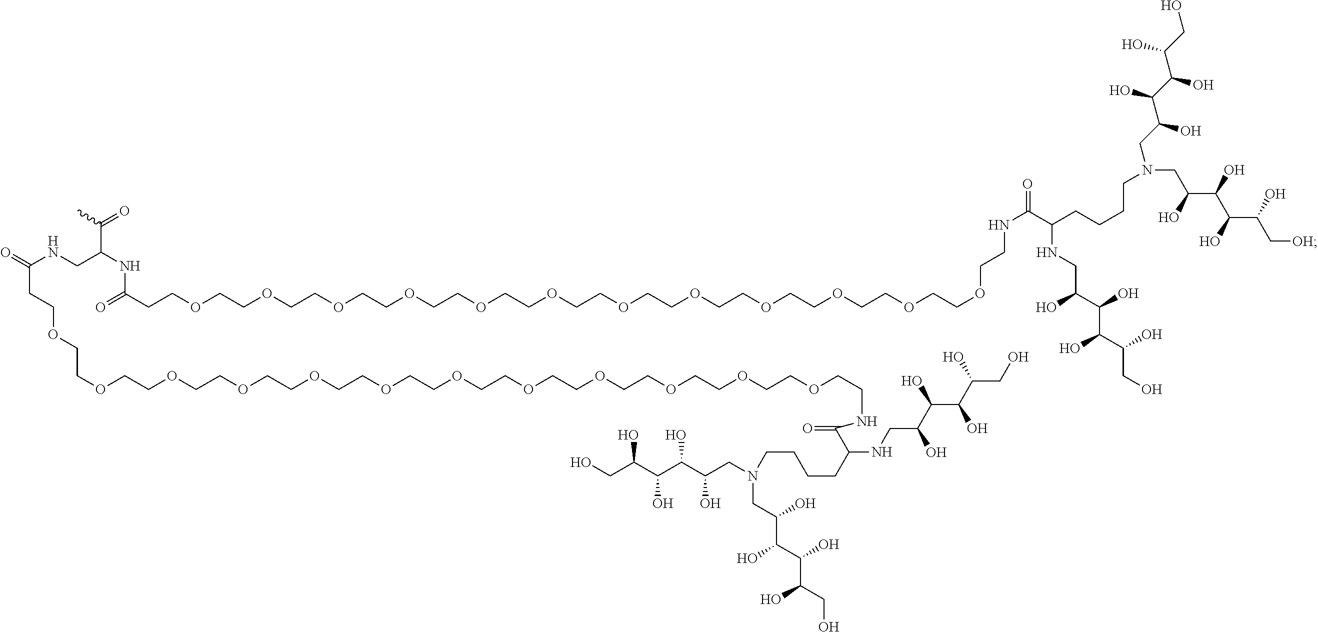

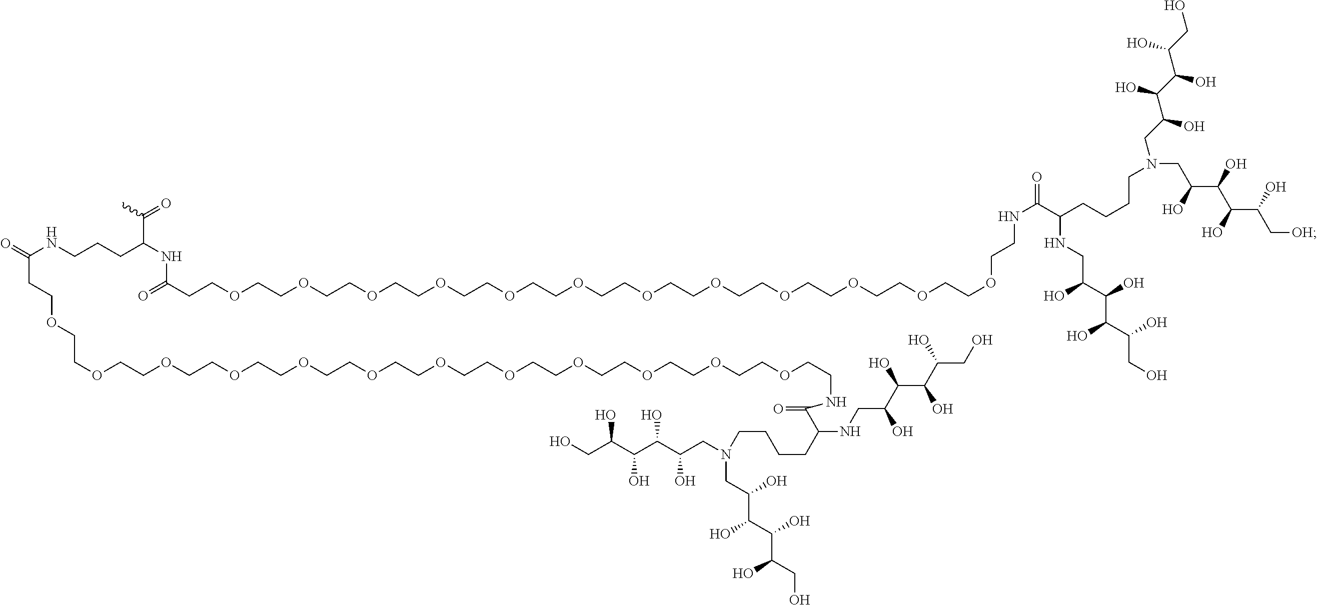

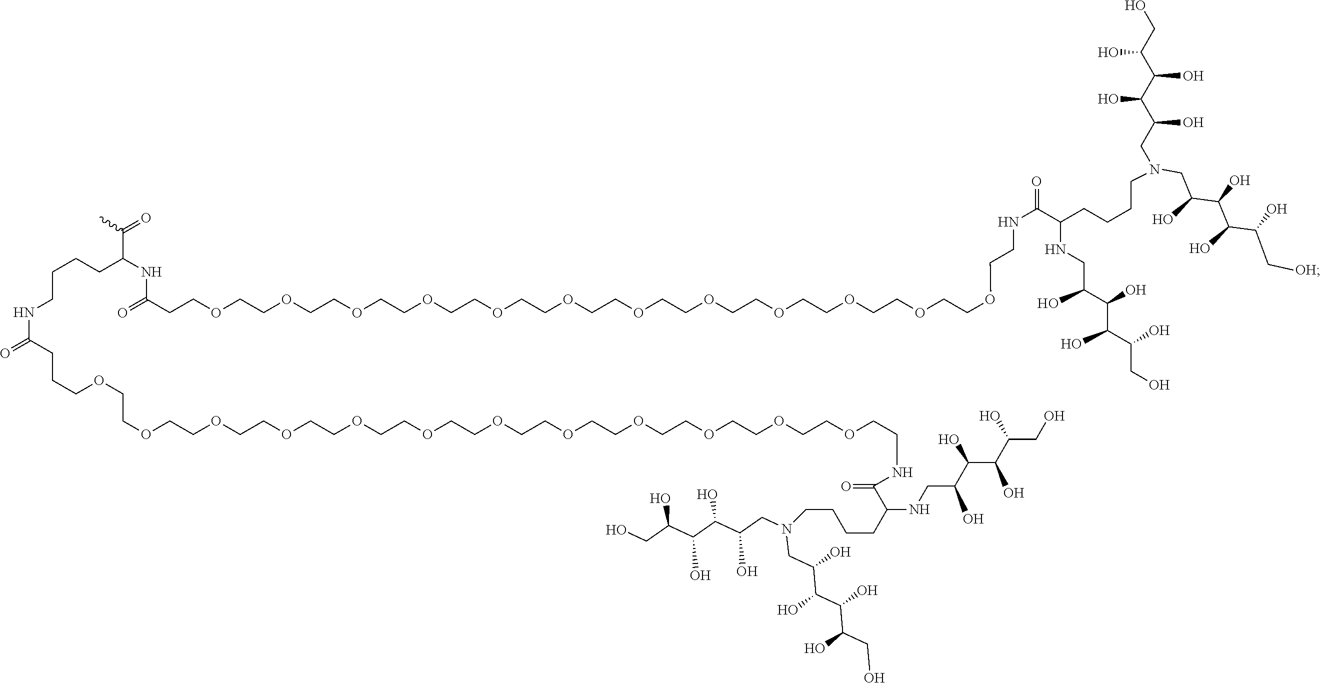

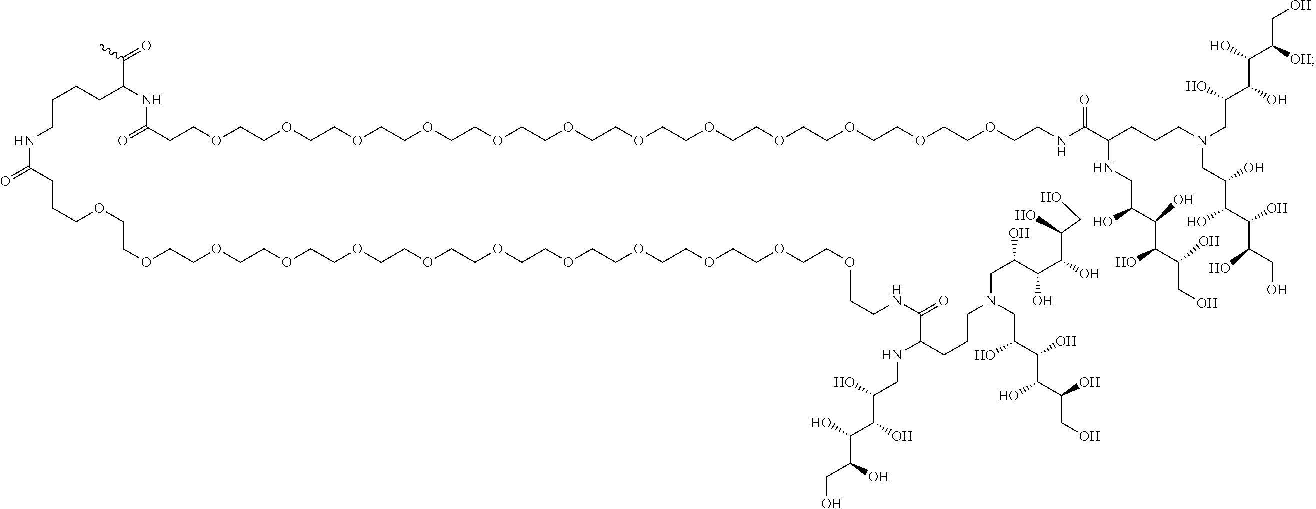

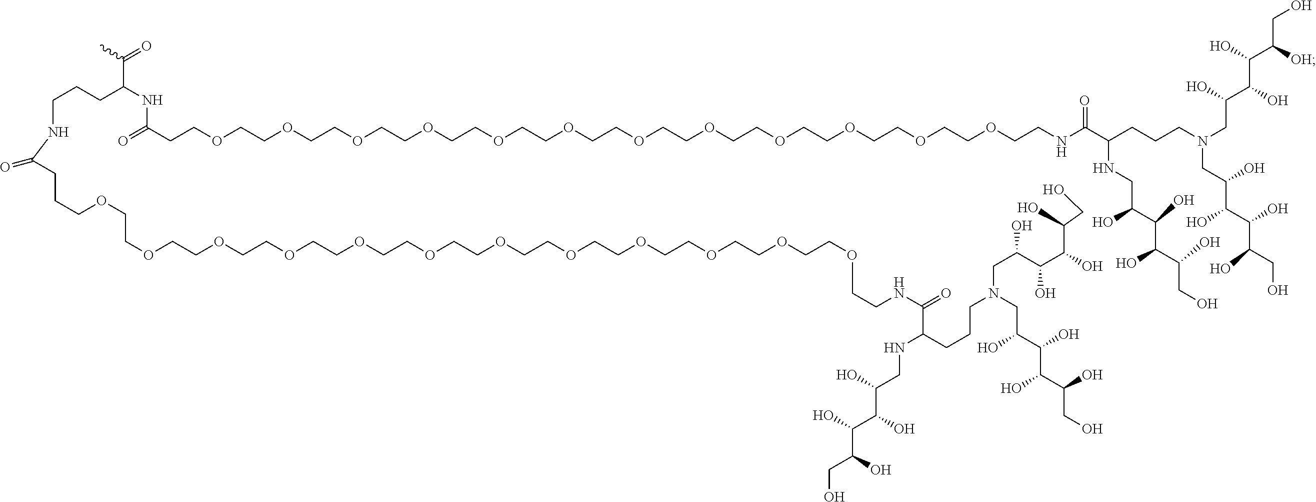

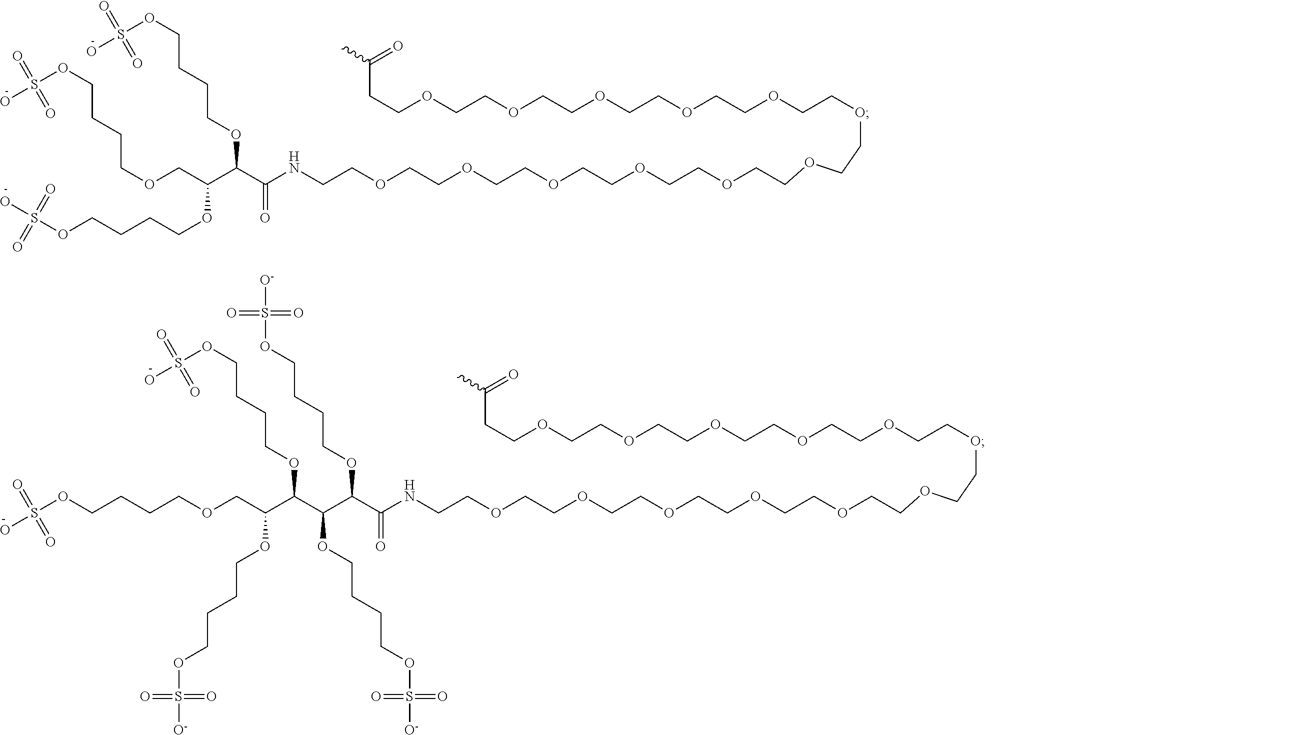

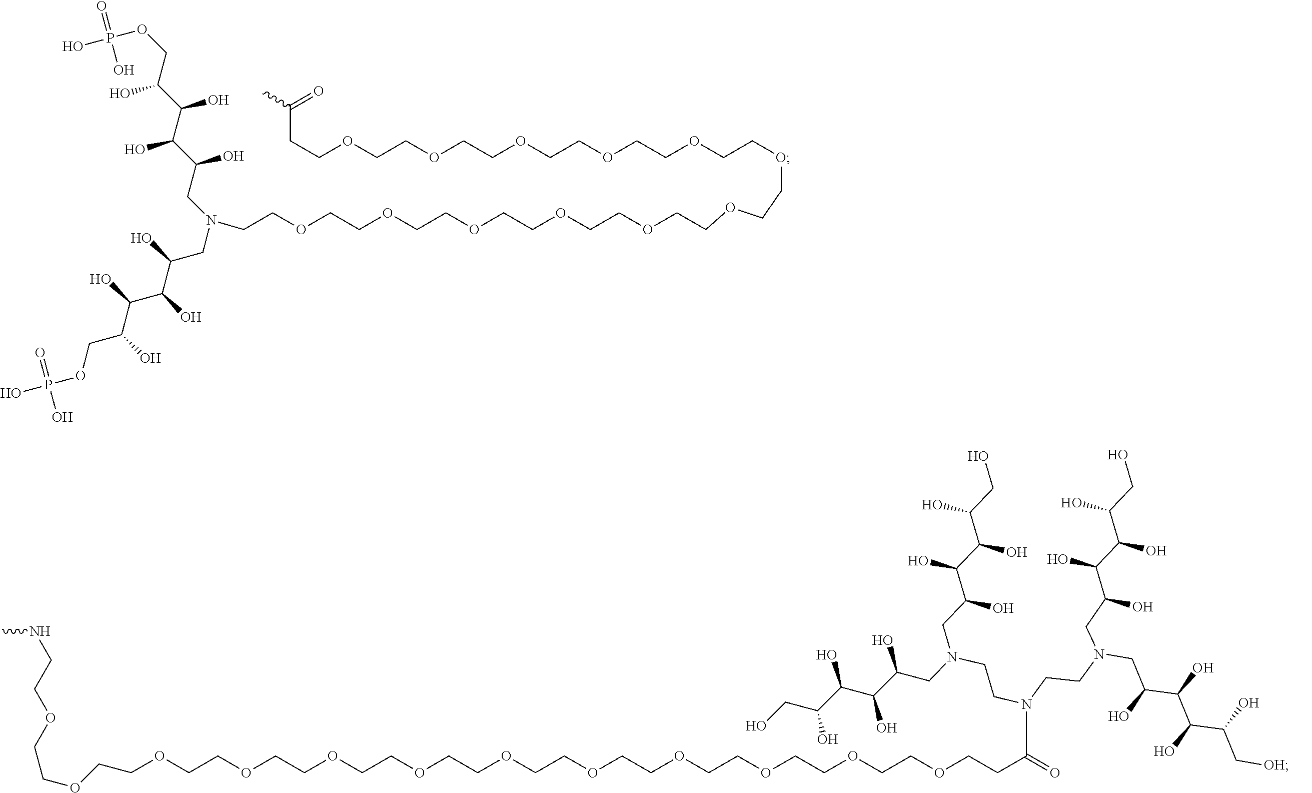

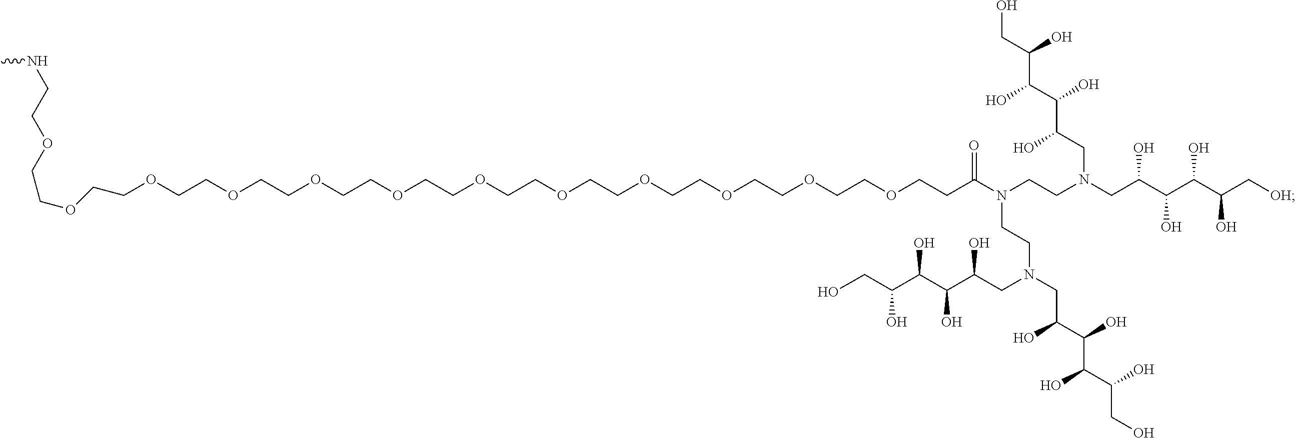

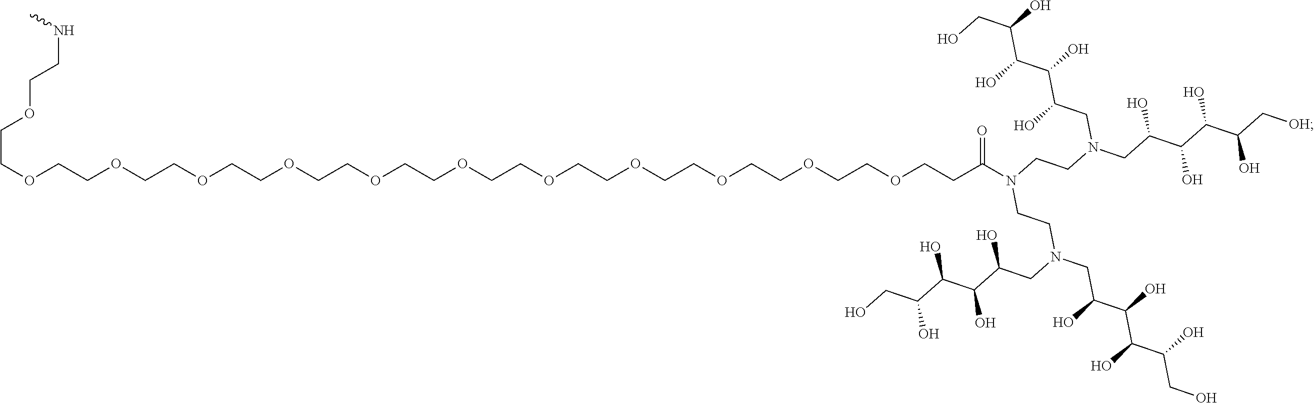

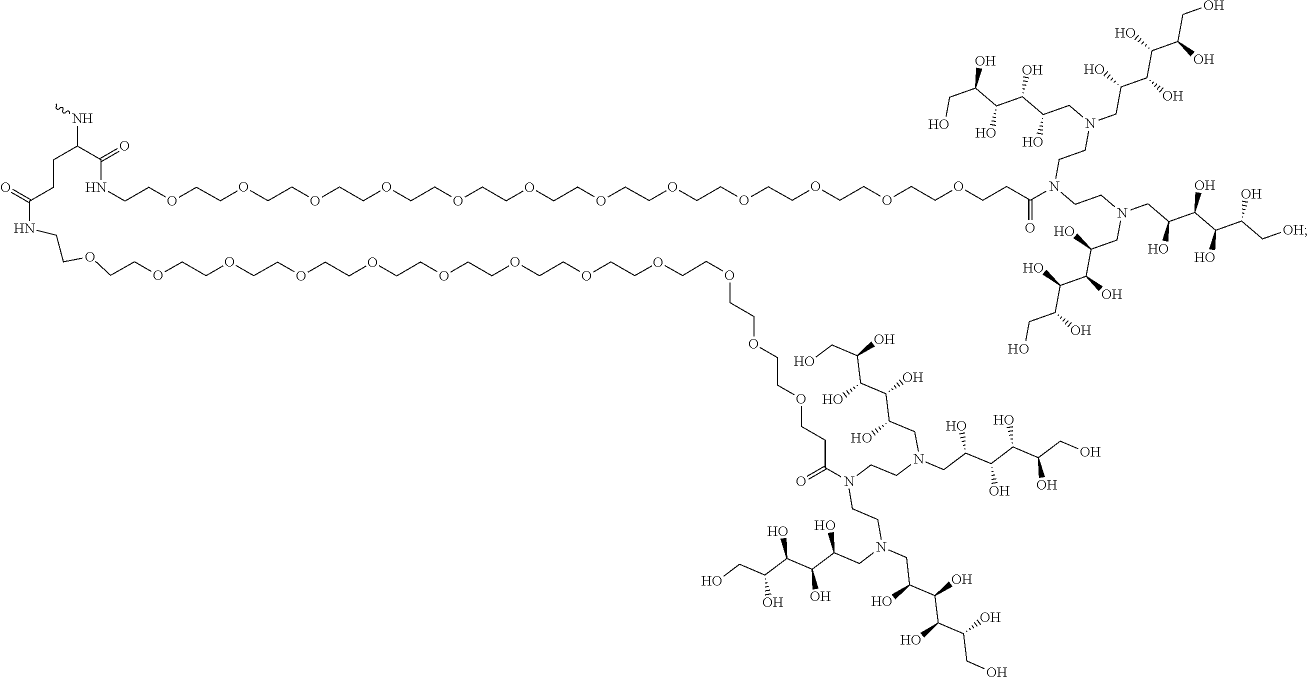

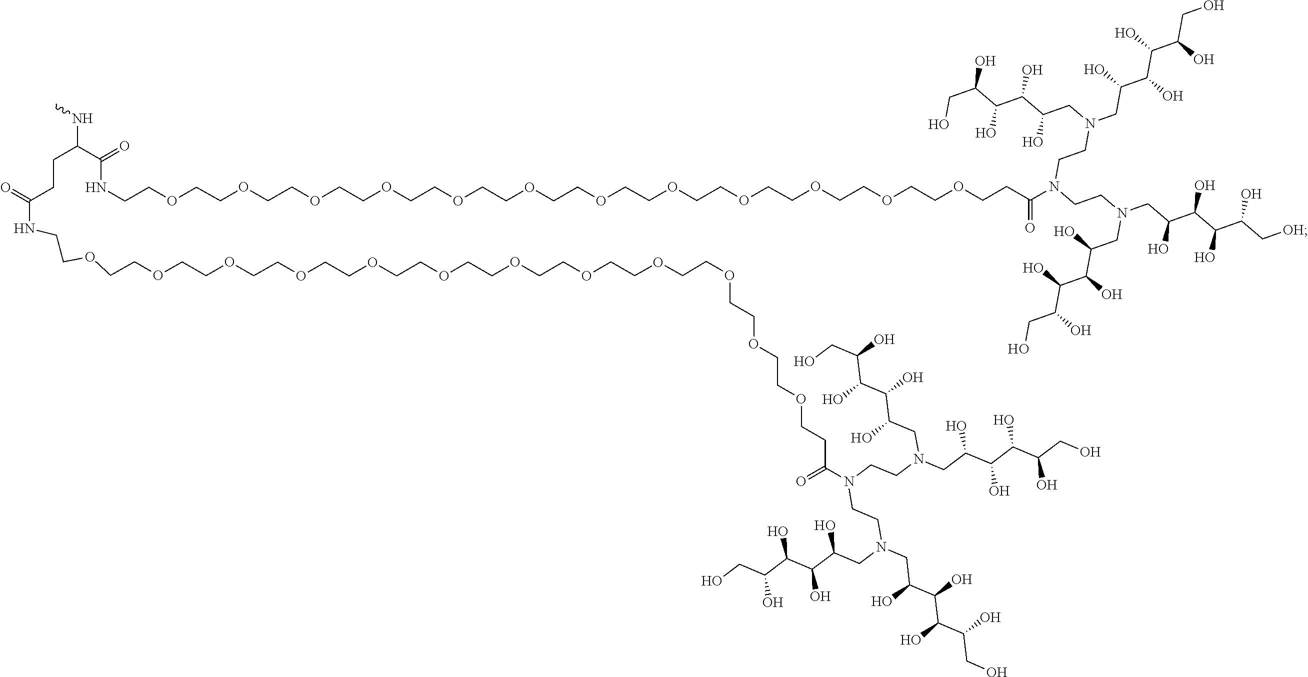

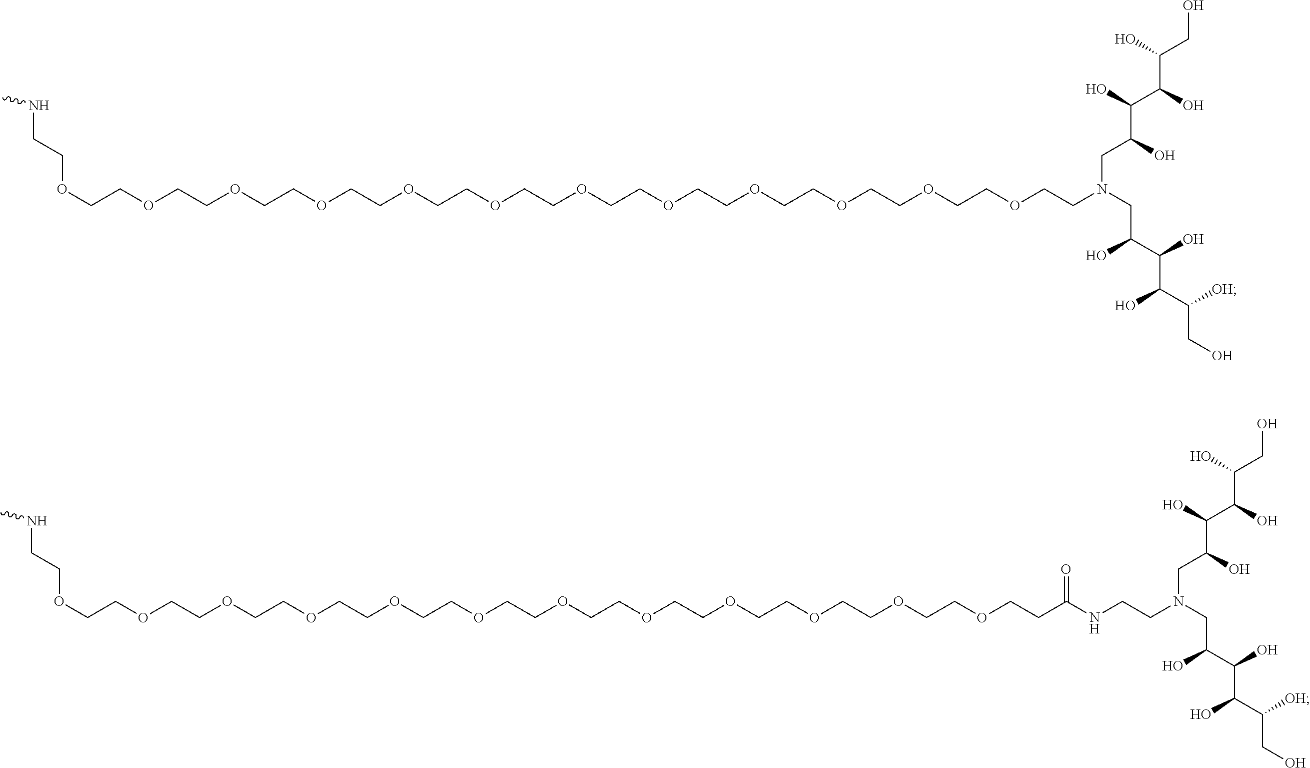

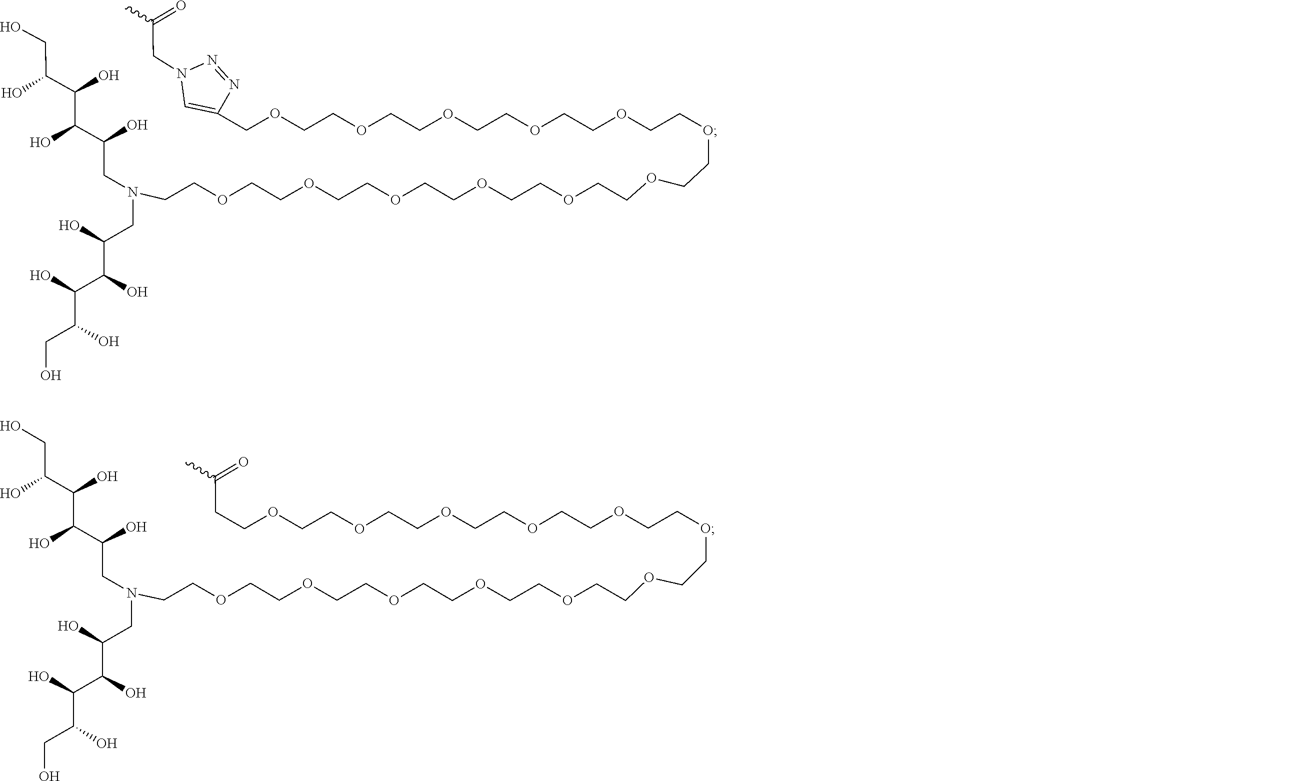

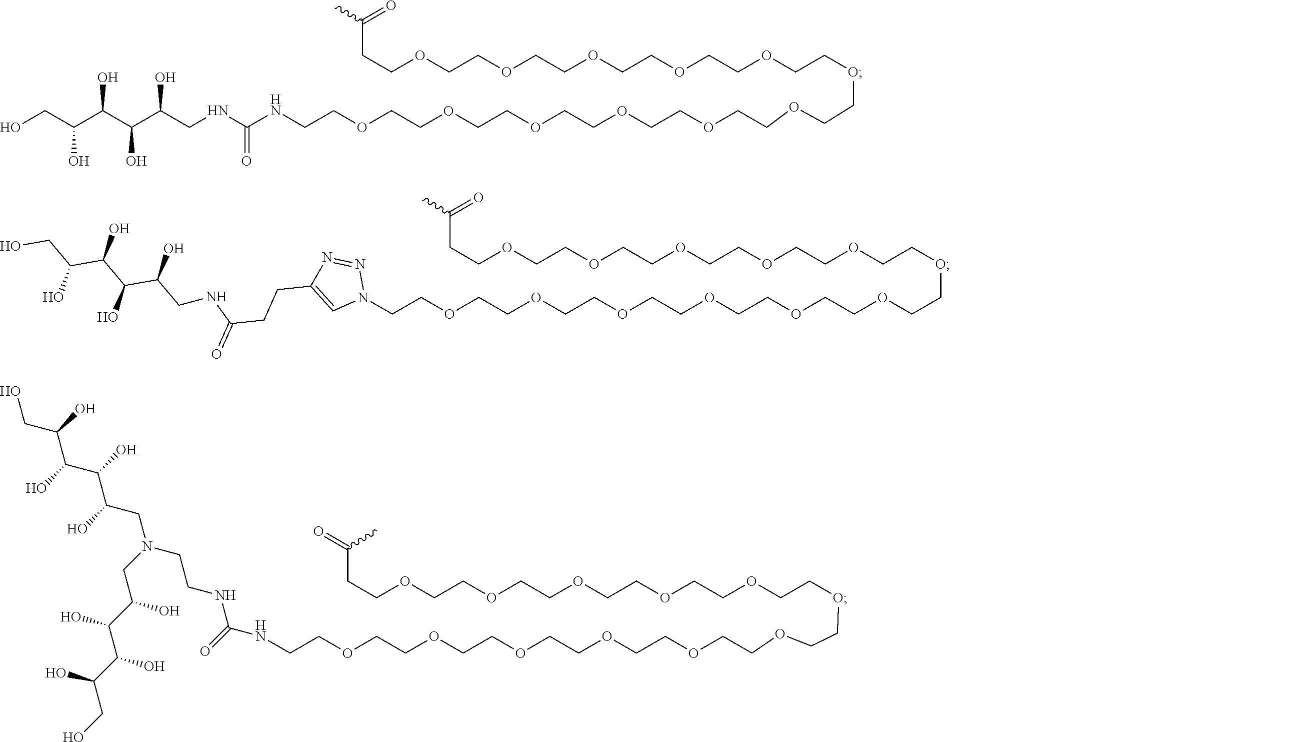

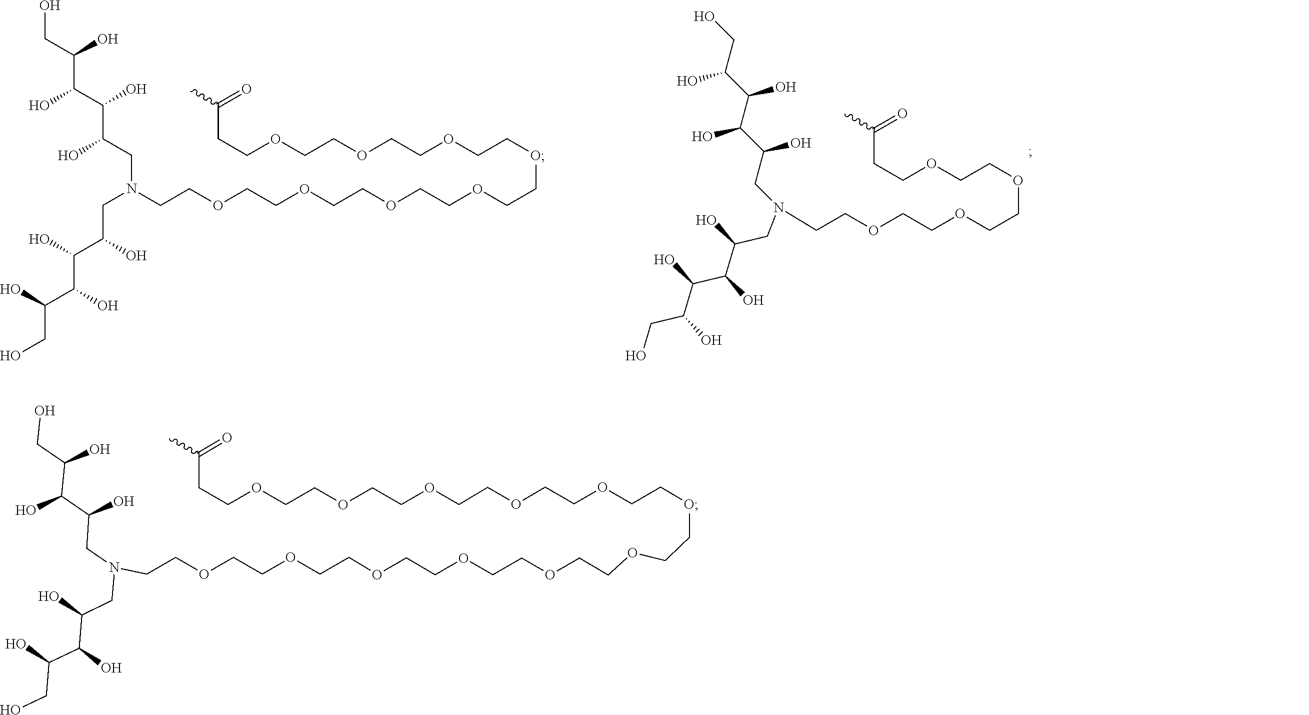

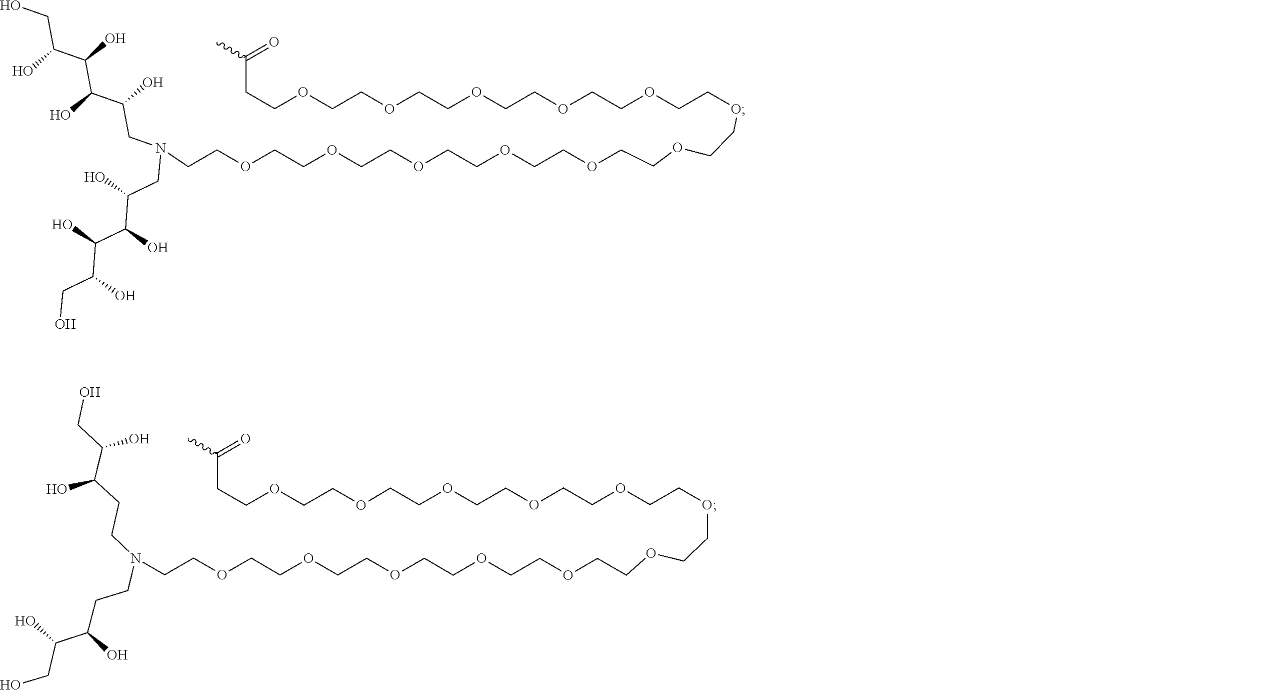

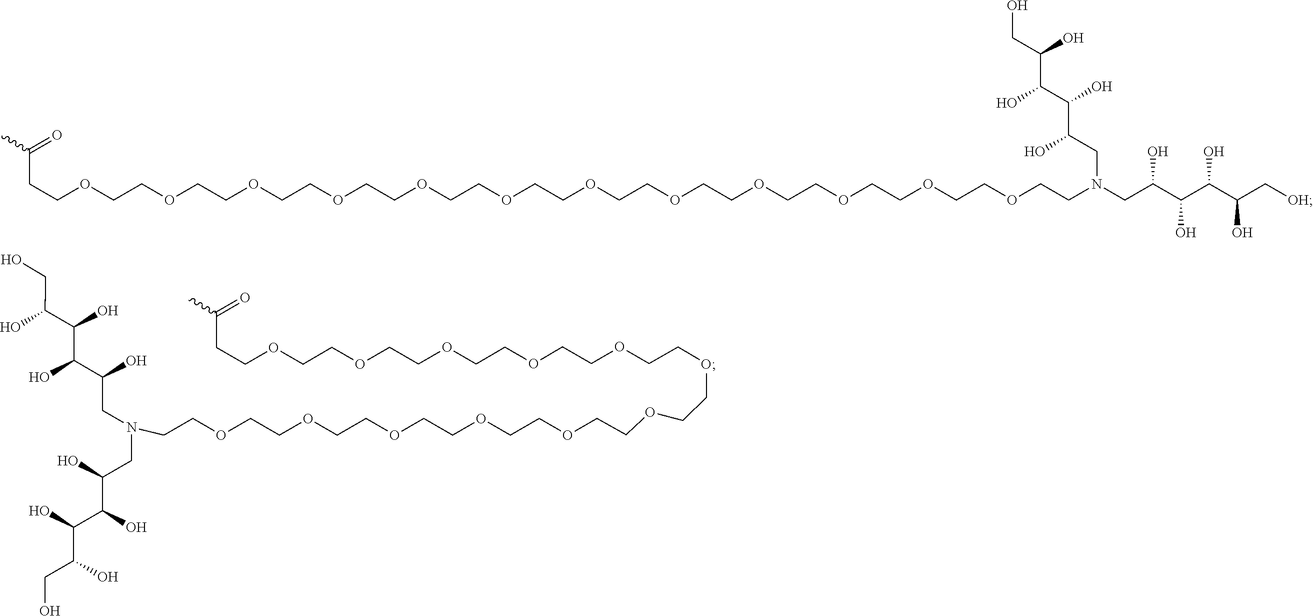

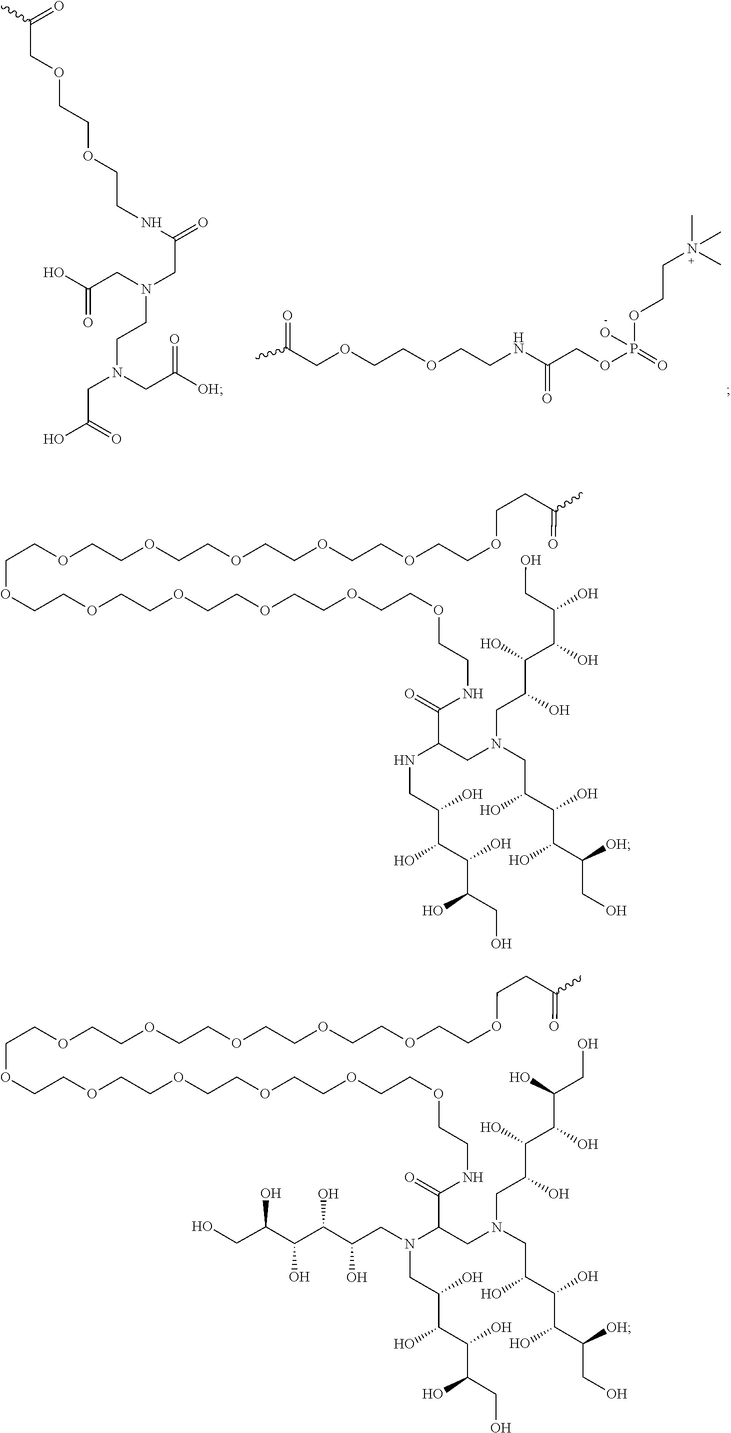

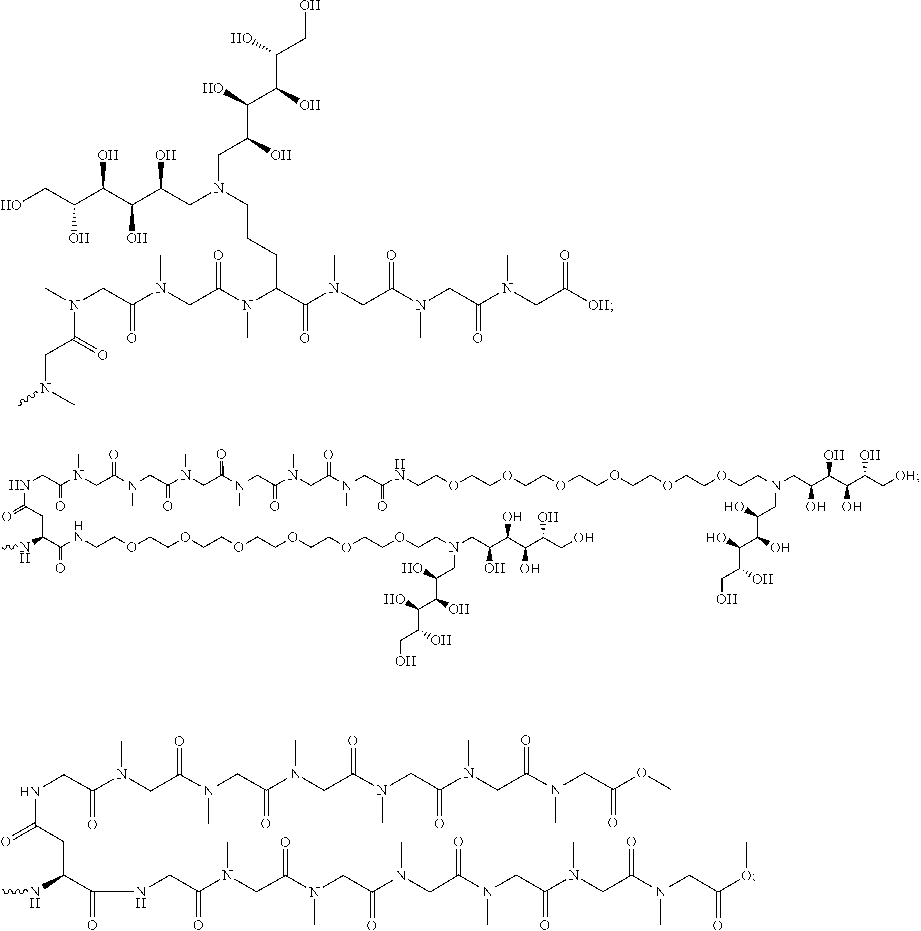

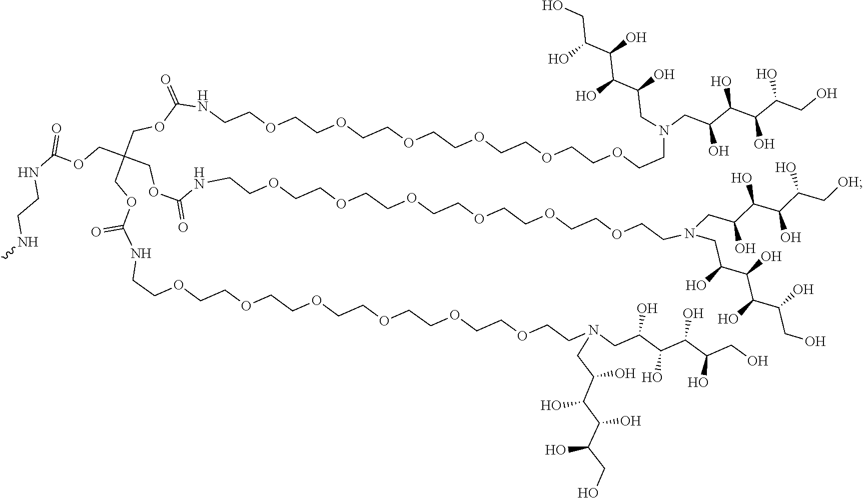

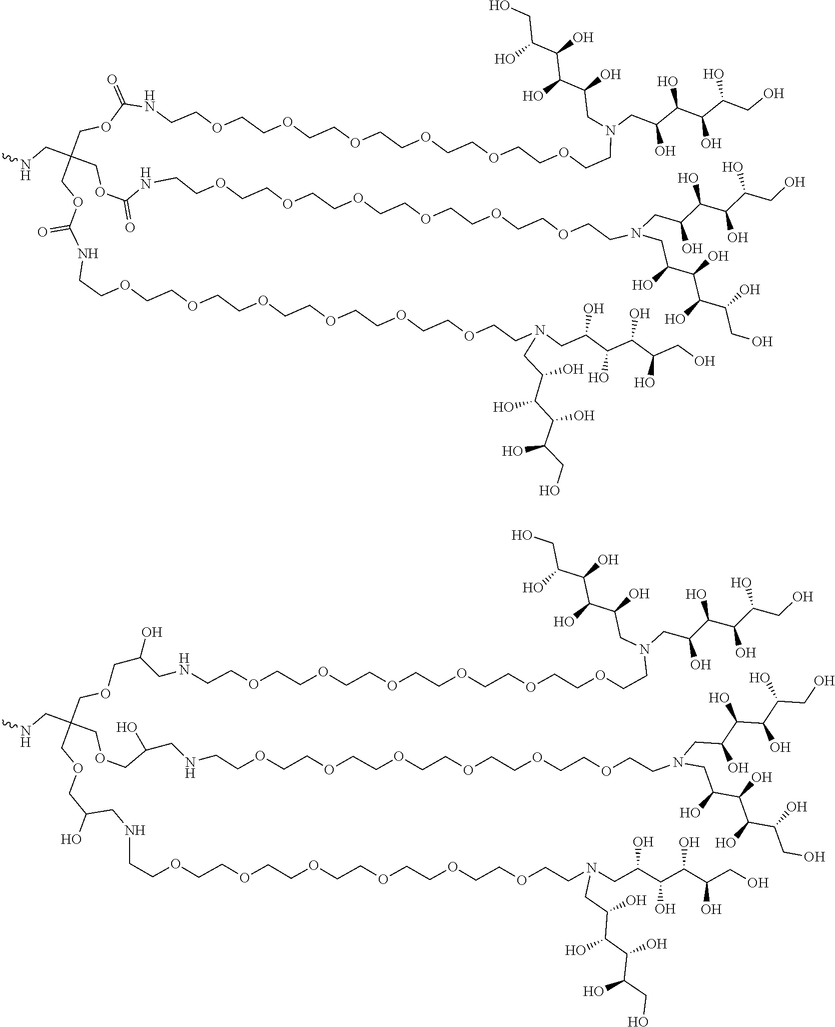

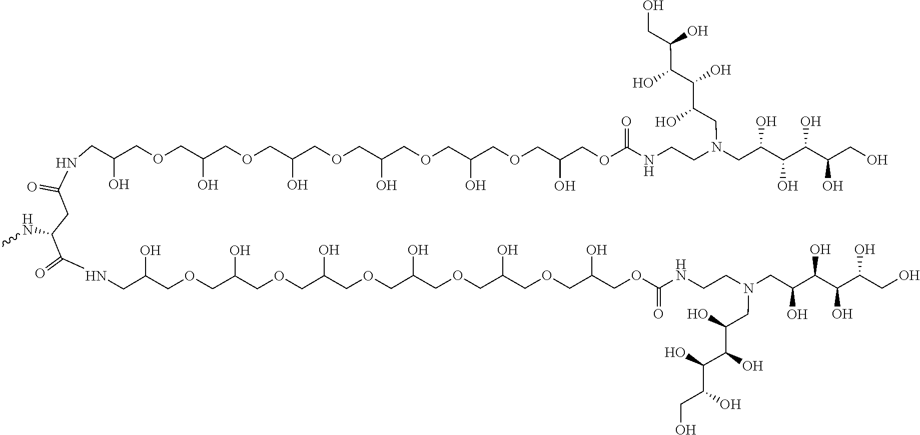

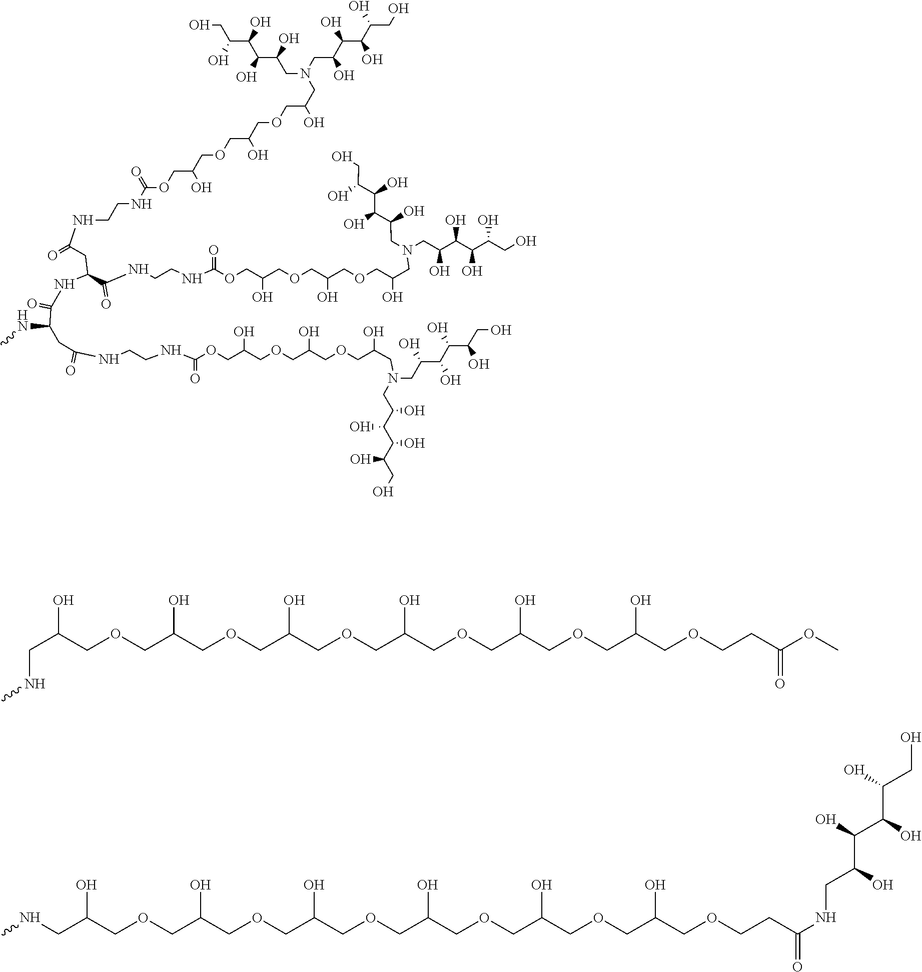

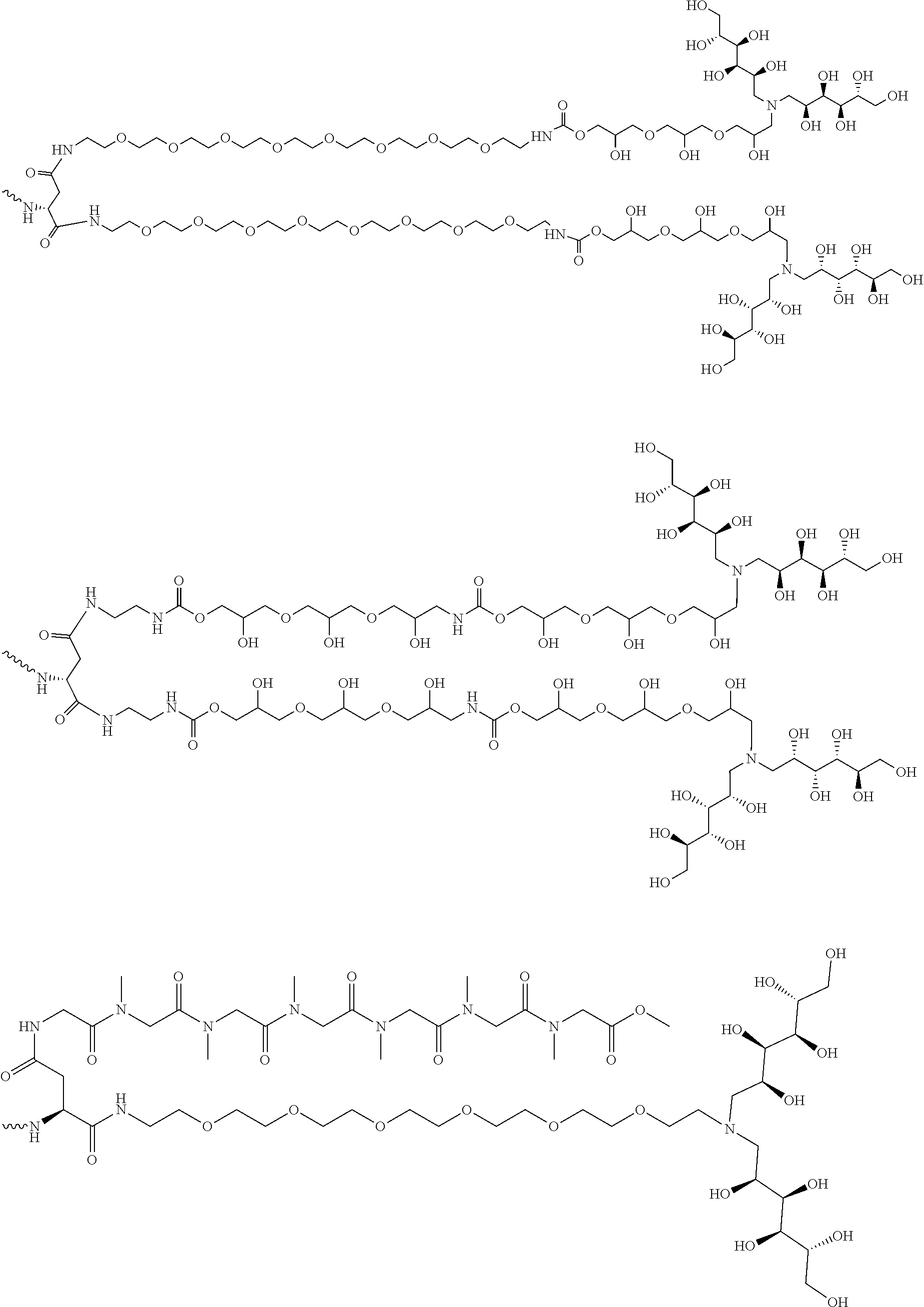

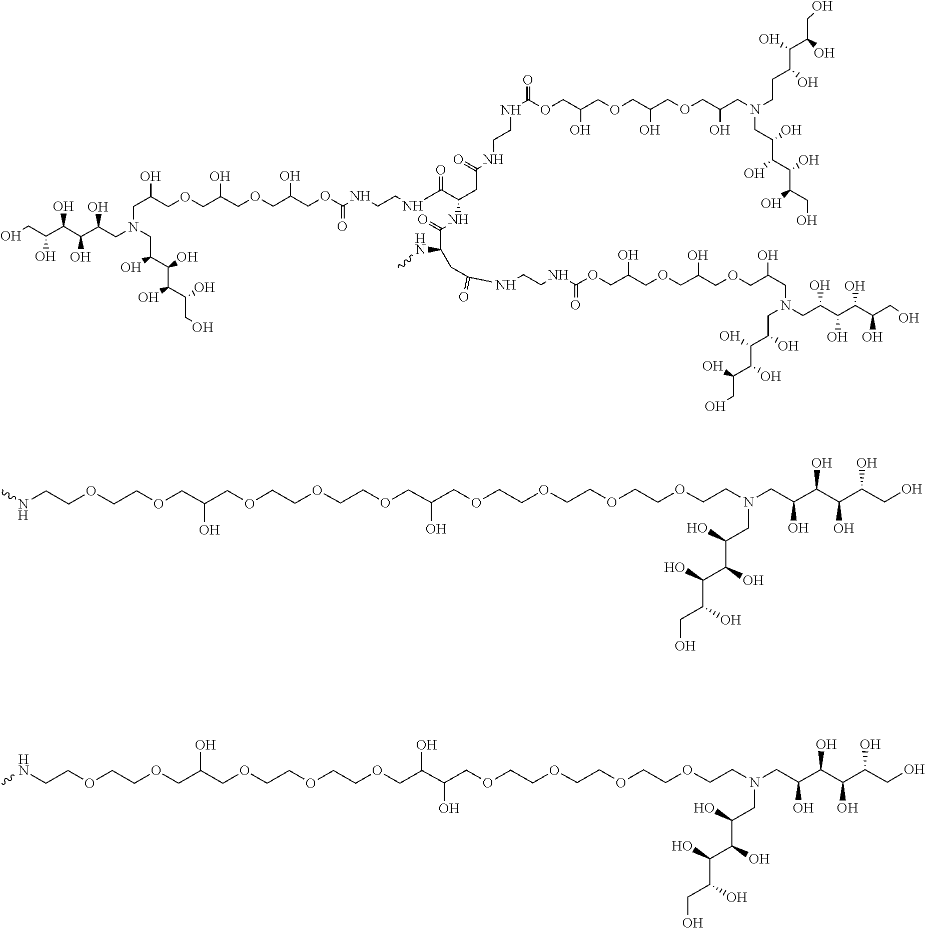

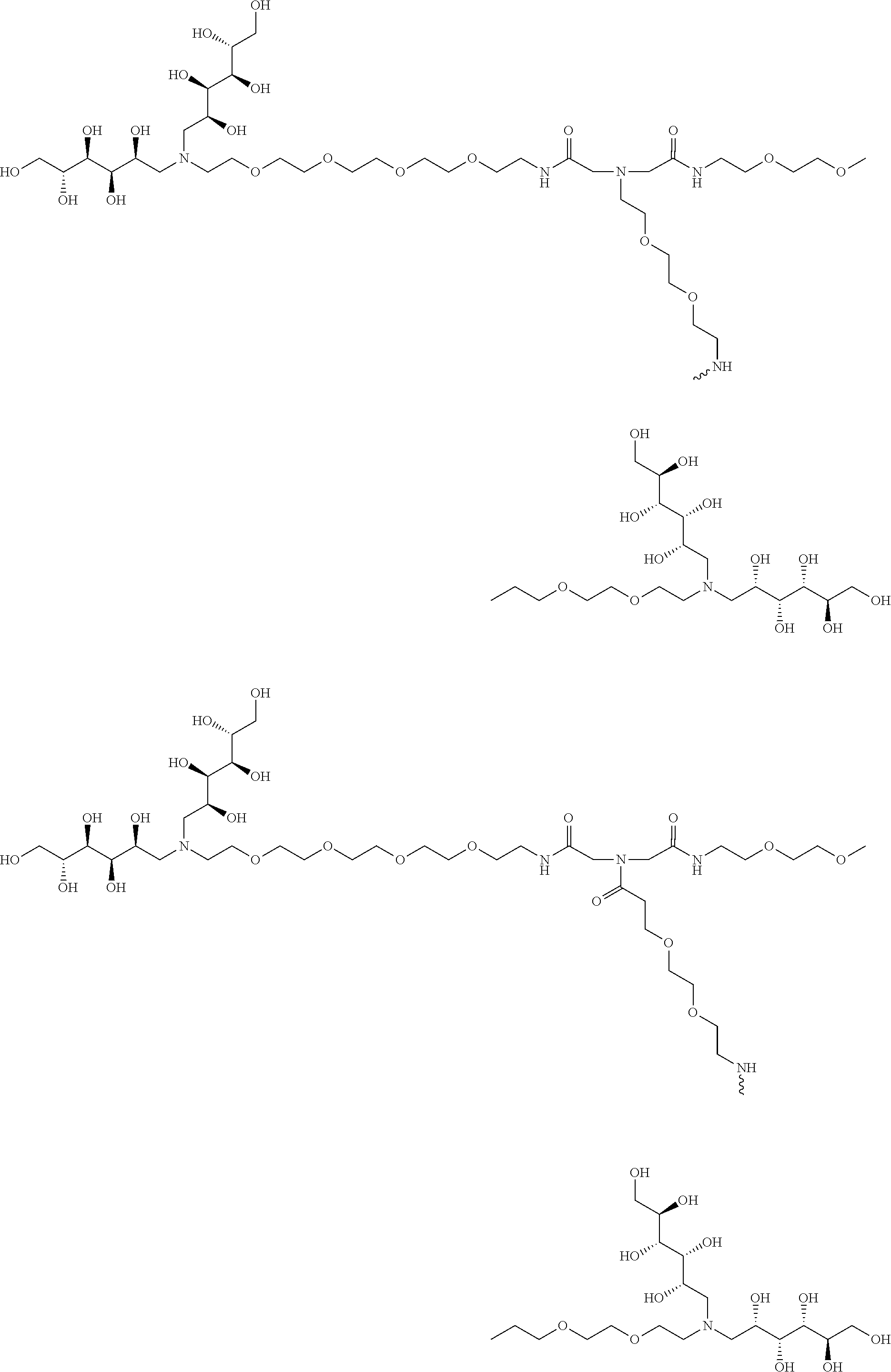

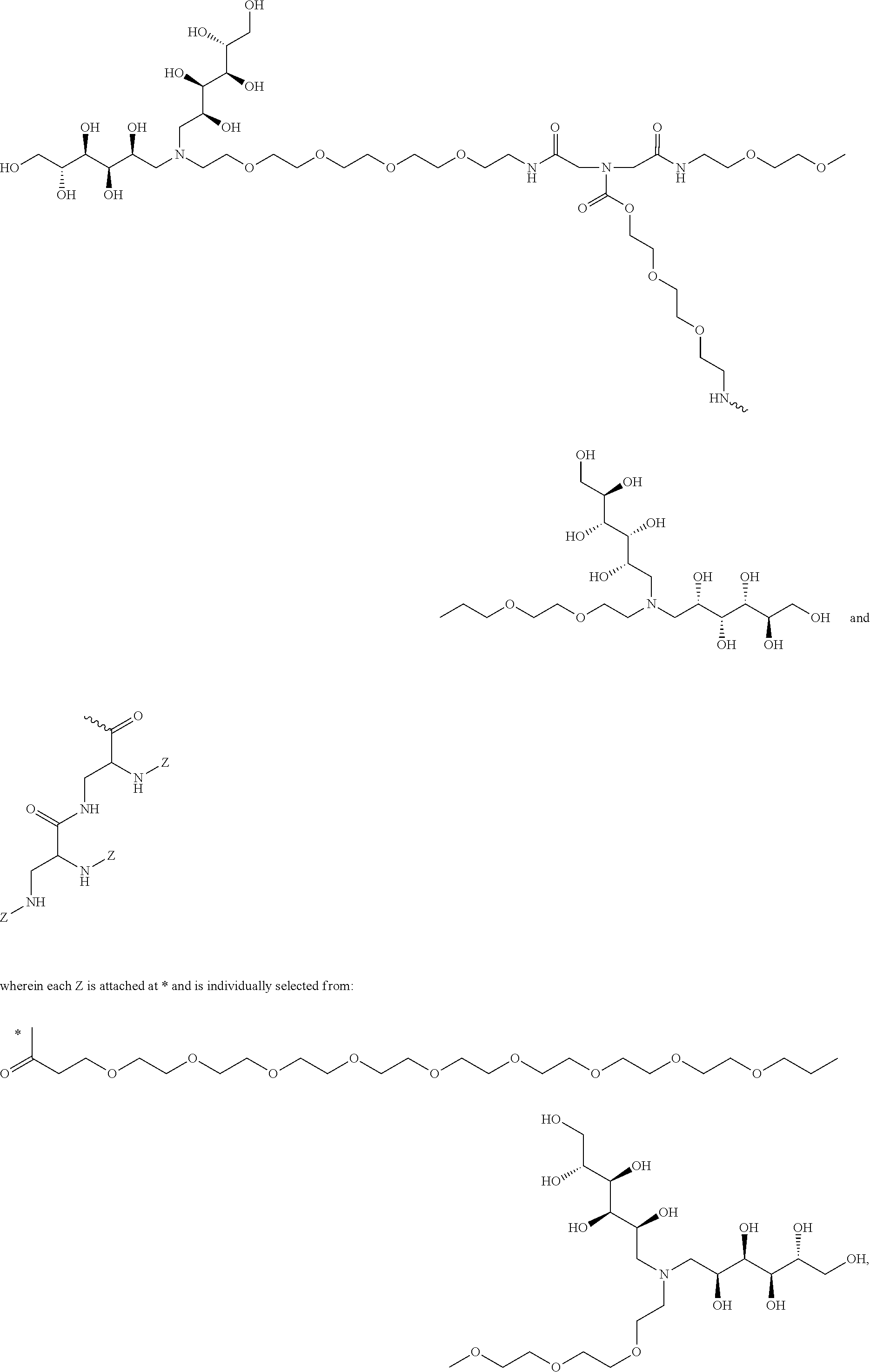

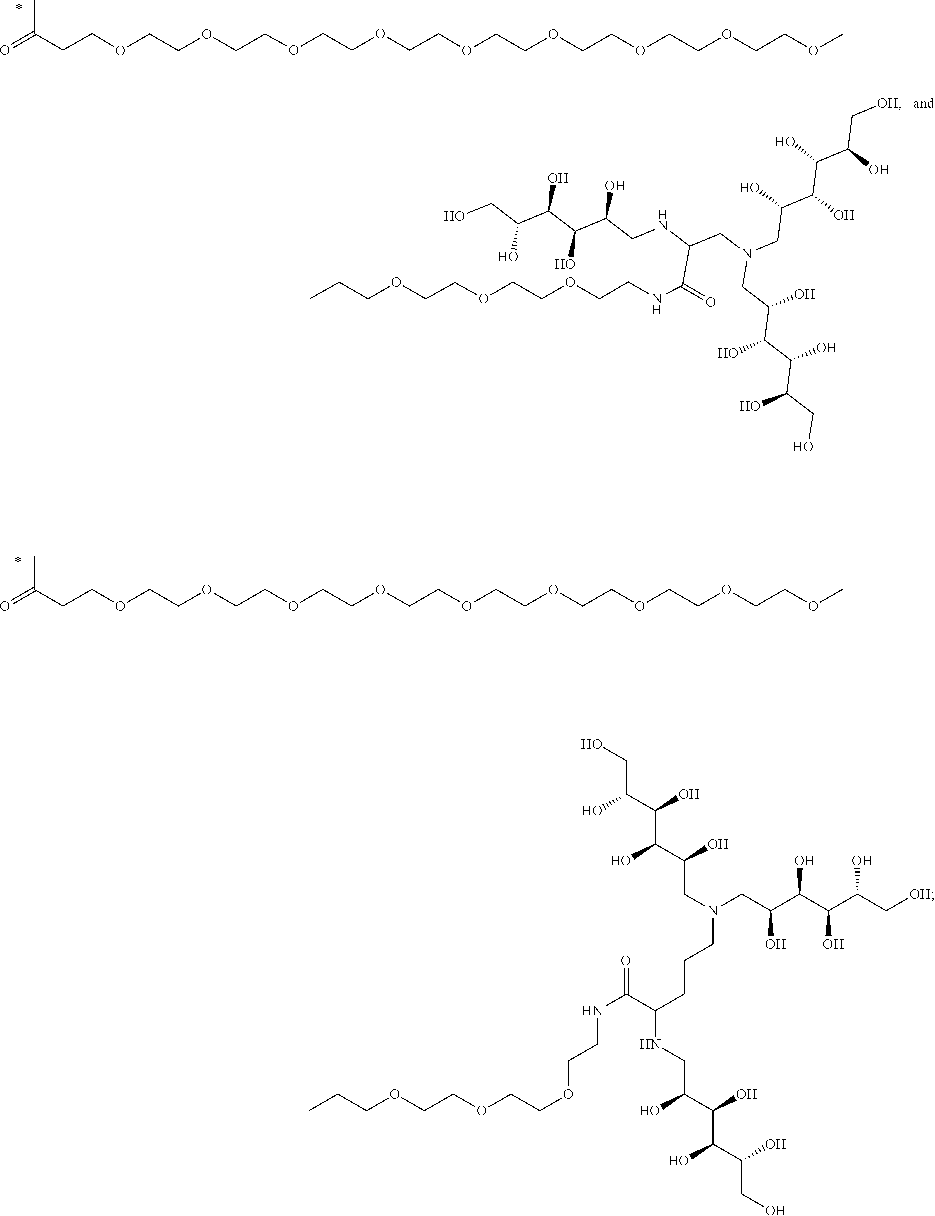

or a stereoisomer or salt thereof, wherein each [symbol] is an attachment to the Amino Acid unit.

In some embodiments, provided is a Linker compound, wherein the Polar group is selected from the following:

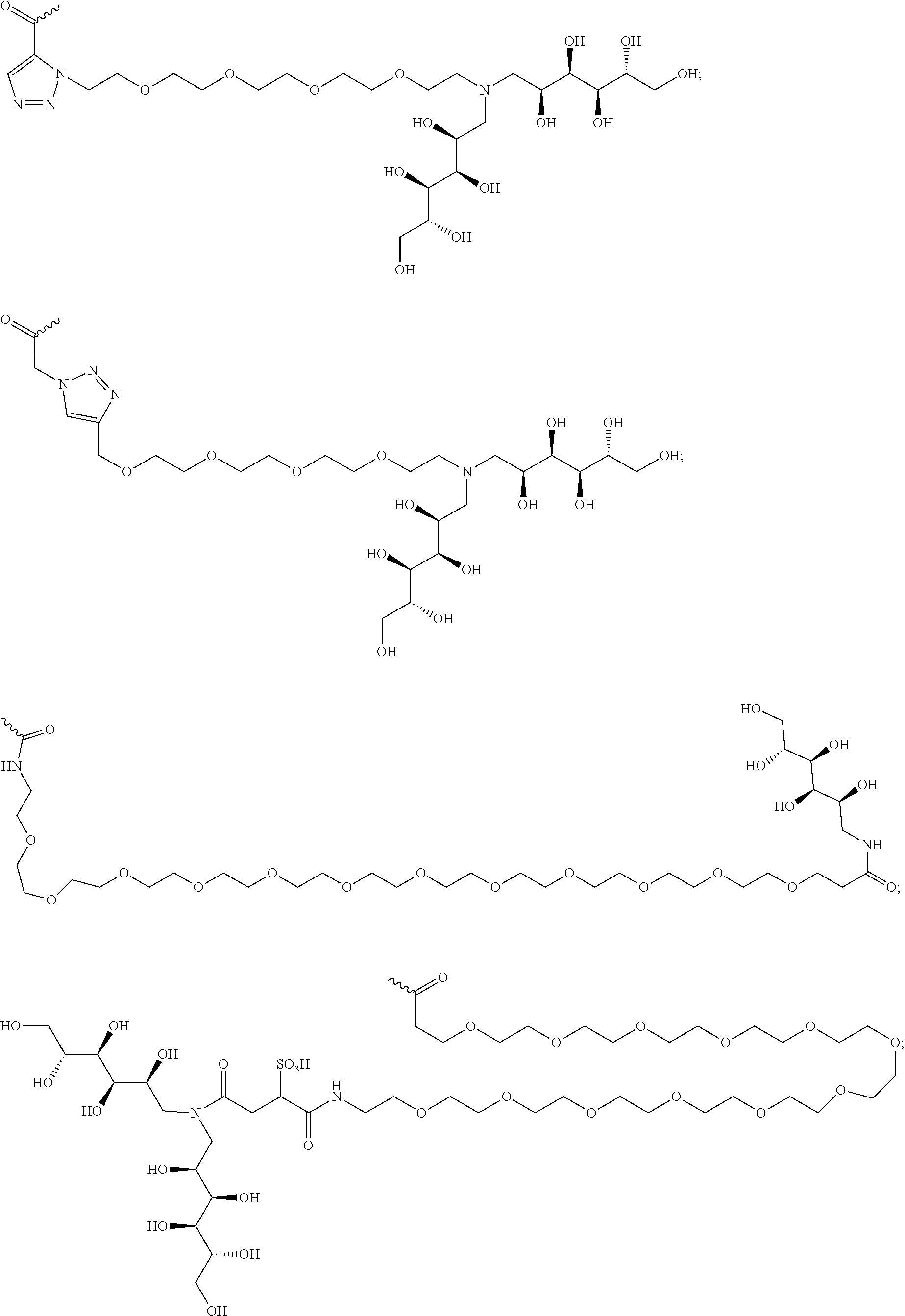

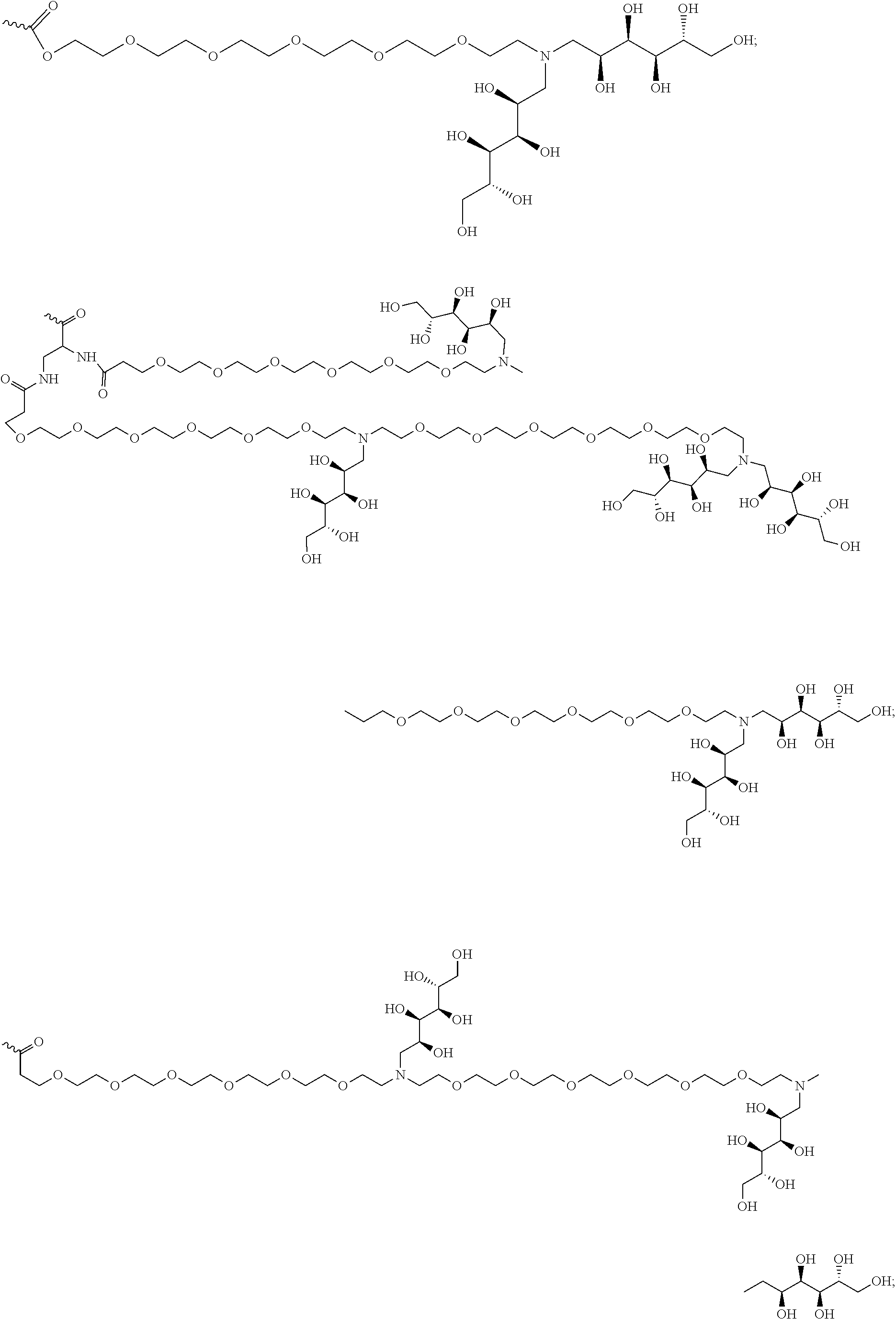

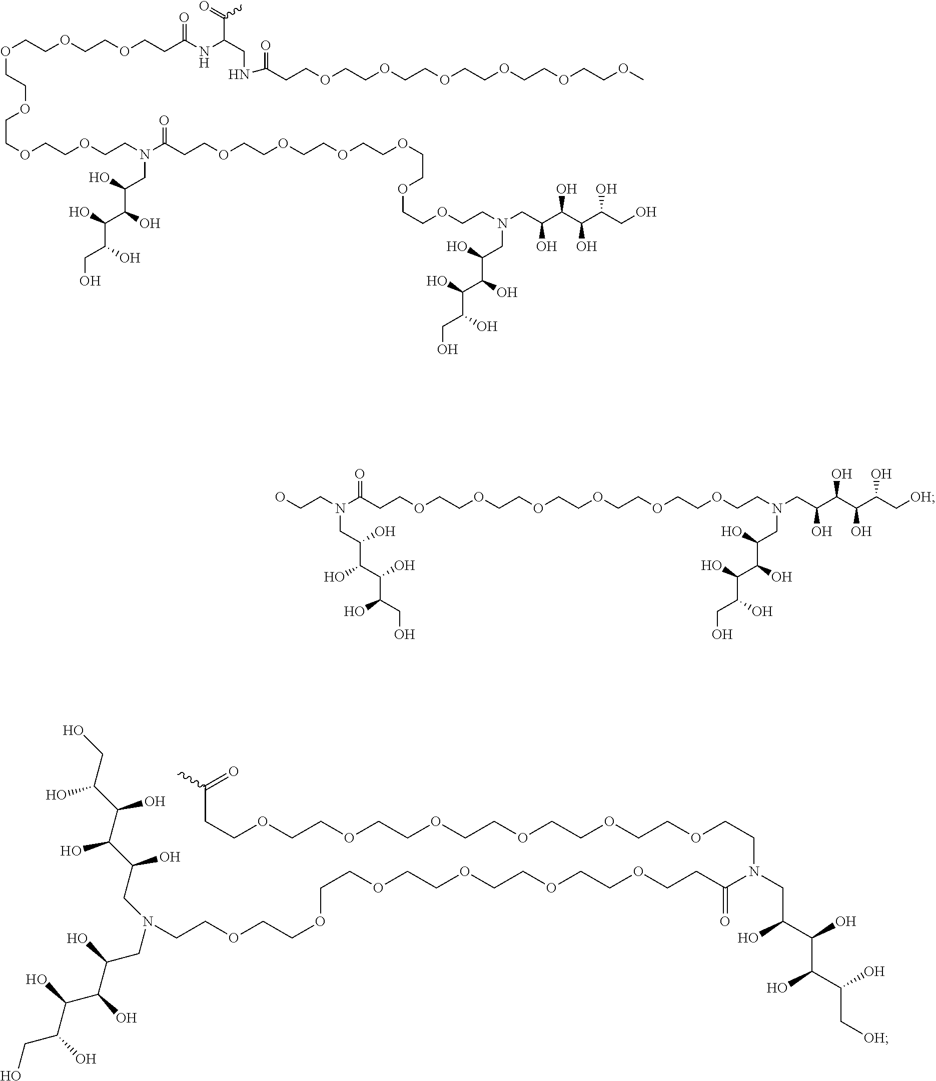

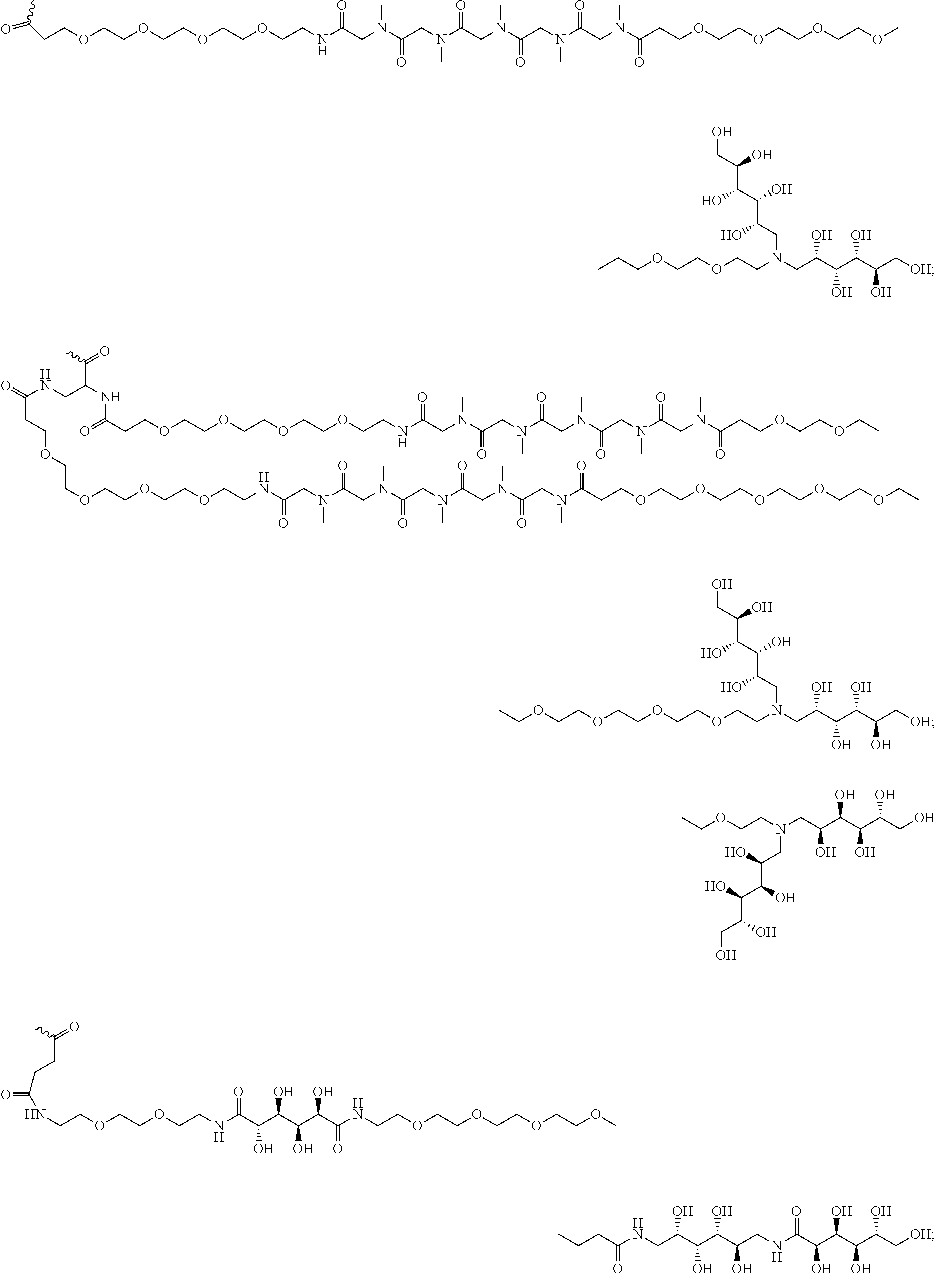

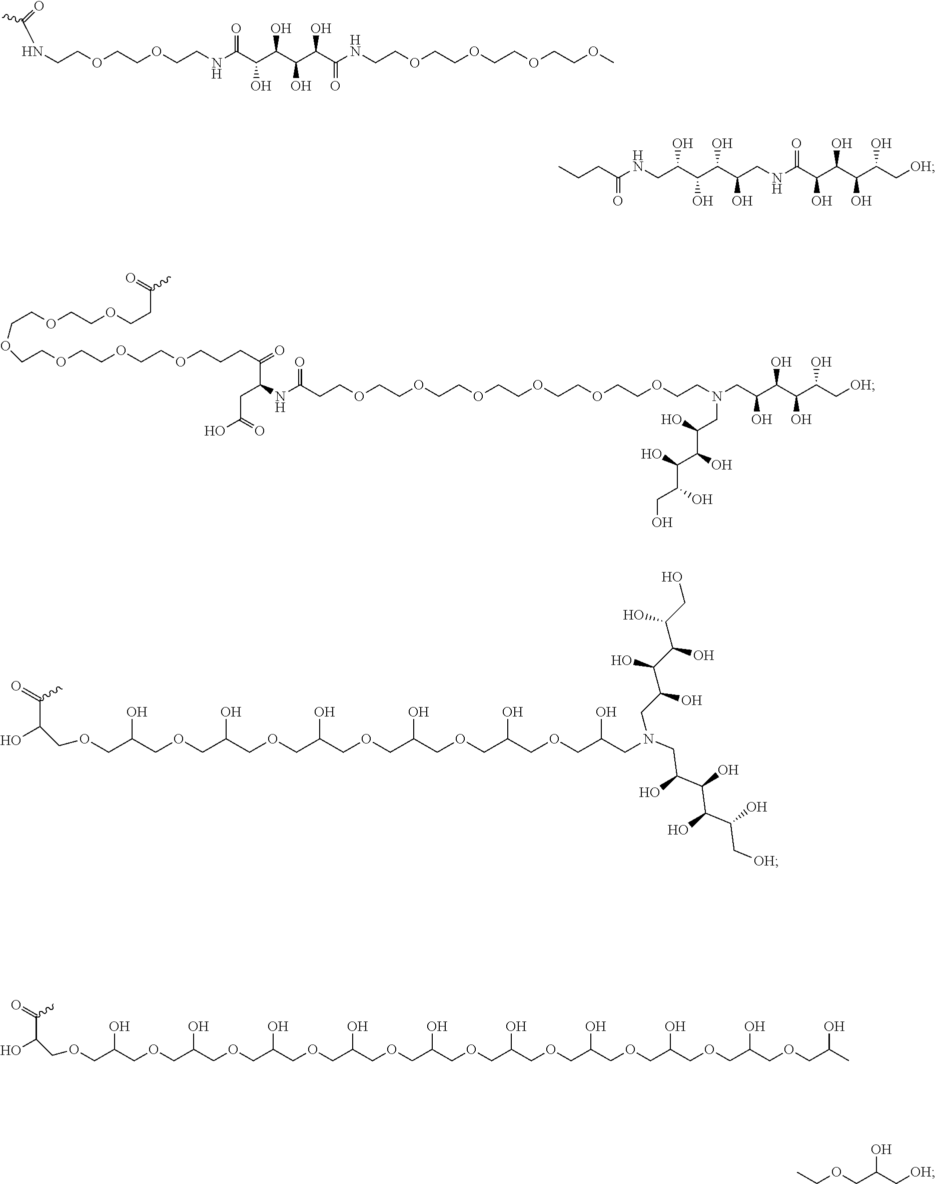

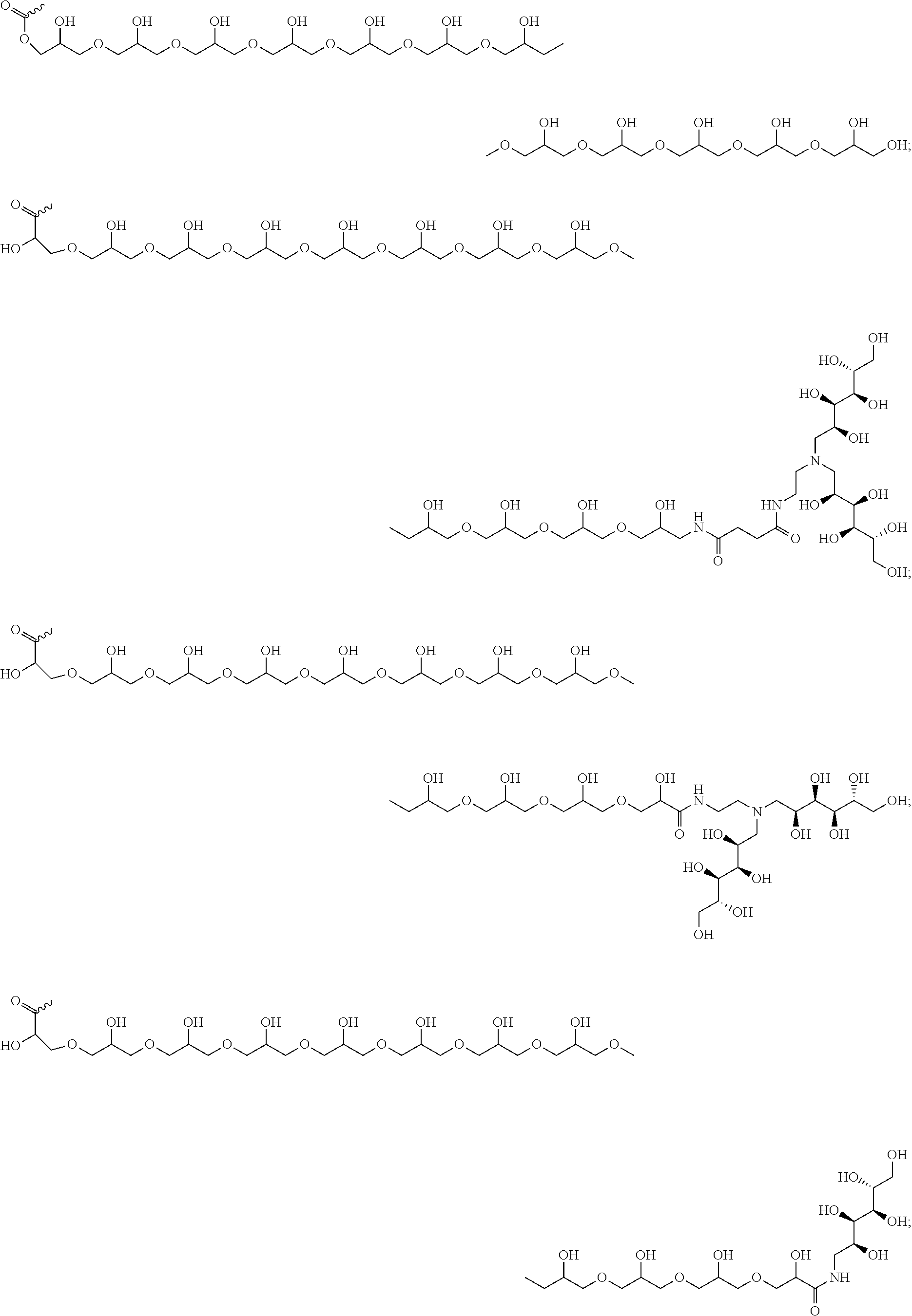

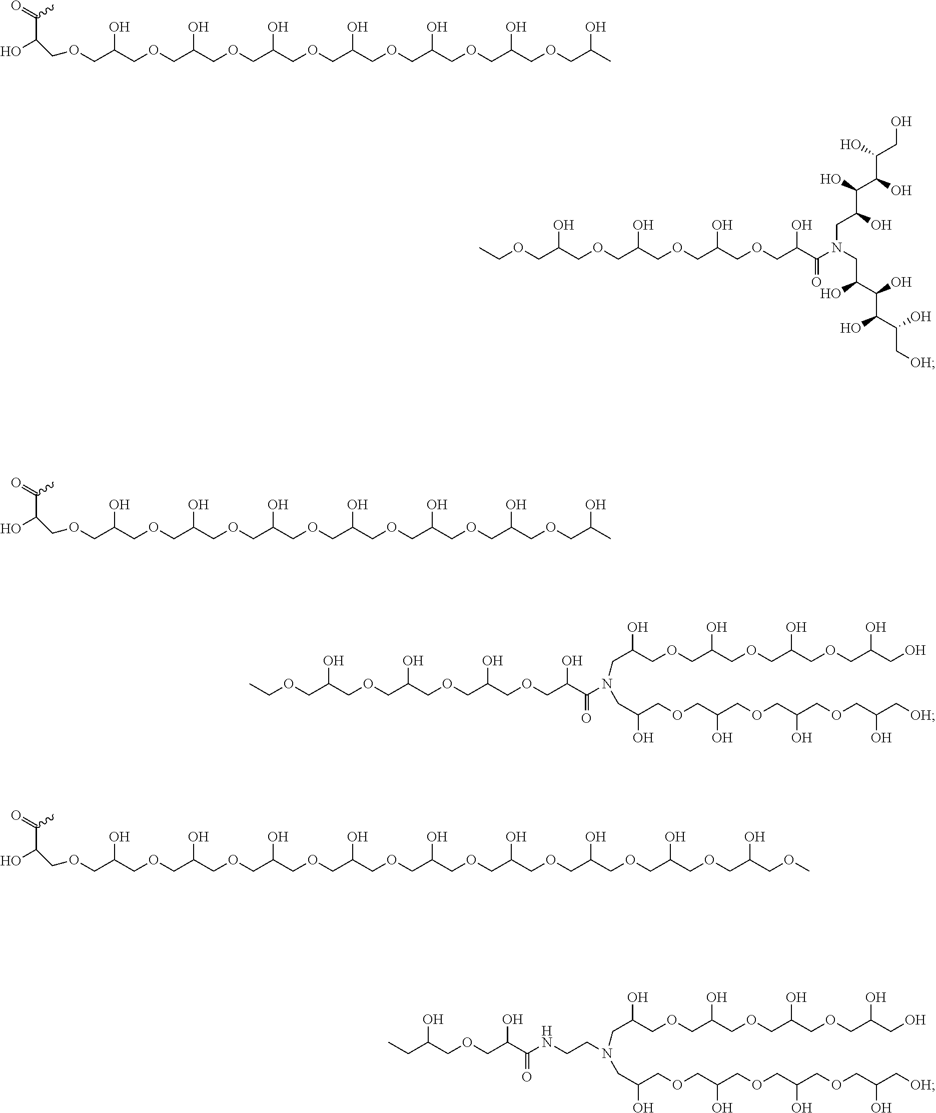

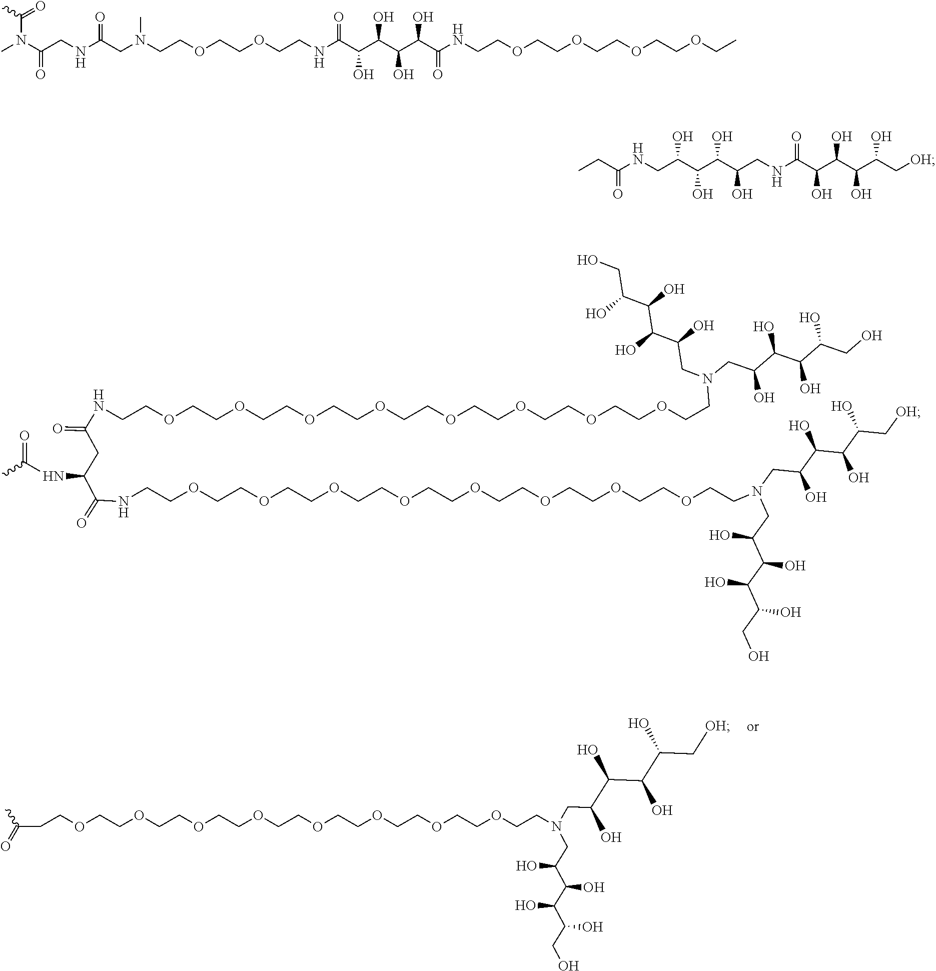

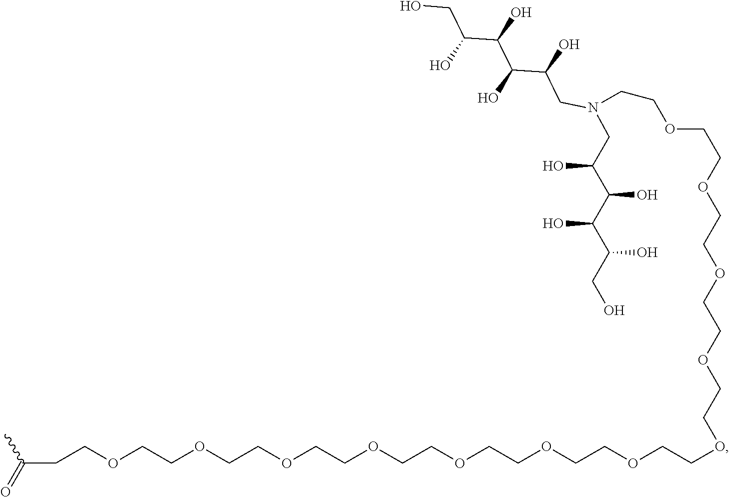

or a stereoisomer thereof, wherein each [symbol] indicates an attachment site of the Amino Acid unit.

In some embodiments, provided is a Linker compound, wherein the Polar group comprises at least one Carboxyl unit having the following formula:

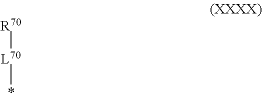

or a stereoisomer or salt thereof, wherein:
(a)
   L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment to the Amino Acid unit, or to a remainder of the Polar group;
   R⁷⁰ is ˜NR⁷¹(R⁷²—R⁷³), wherein R⁷¹ is selected from H, C₁-C₁₂ alkyl, substituted C₁-C₁₂ alkyl, or polyethylene glycol (optionally having 1 to 12 ethylene glycol subunits), R⁷² is a bond or is selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and R⁷³ is a carboxyl or polycarboxyl, wherein the polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide;
(b)
   L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment to the Amino Acid unit, or to a remainder of the Polar group;
   R⁷⁰ is ˜NR⁷¹(R⁷⁵-(R⁷³)₂), wherein R⁷¹ is selected from H, C₁-C₁₂ alkyl, substituted C₁-C₁₂ alkyl, or polyethylene glycol (optionally having 1 to 12 ethylene glycol subunits), R⁷⁵ is a branched optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl and each R⁷³ is independently carboxyl or polycarboxyl, wherein the polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide; or
(c)
   L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment to the Amino Acid unit, or to a remainder of the Polar group;
   R⁷⁰ is ˜N(R⁷⁴—R⁷³)(R⁷²—R⁷³), wherein R⁷² and R⁷⁴ are each independently selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and each R⁷³ is independently carboxyl or polycarboxyl, wherein the polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide.

In some embodiments, provided is a Linker compound, comprising a Polar group including the Polymer unit and a Sugar unit.

In some embodiments, provided is a Linker compound, comprising a Polar group including at least two Polymer units.

In some embodiments, provided is a Linker compound, comprising a Polar group including the Polymer unit(s) and a Carboxyl unit.

In some embodiments, provided is a Linker compound, comprising at least two Polar groups.

In some embodiments, provided is a Linker compound, comprising a Polar group including the Polymer unit, the Sugar unit and the Carboxyl unit.

In some embodiments, provided is a Linker compound, comprising a Polar group including at least two Polymer units, at least one Sugar unit and at least one Carboxyl unit.

In some embodiments, provided is a Linker compound, wherein the Amino Acid unit comprises at least two amino acid subunits.

In some embodiments, provided is a Linker compound, comprising two of the Polar groups, when present, both attached to the Amino Acid unit.

In some embodiments, provided is a Linker compound, wherein the Linker unit is attached to a side chain of a subunit of the Amino Acid unit.

In some embodiments, provided is a Linker compound, wherein the Amino Acid unit is joined to the Linker Unit by a non-peptidic linking group.

In some embodiments, provided is a Linker compound, wherein the non-peptidic linking group is selected from optionally-substituted C₁-C₁₀ alkylene, optionally-substituted C₂-C₁₀ alkenylene, optionally-substituted C₂-C₁₀ alkynylene, or optionally-substituted polyethylene glycol.

In some embodiments, provided is a Linker compound, comprising one of the following structures:

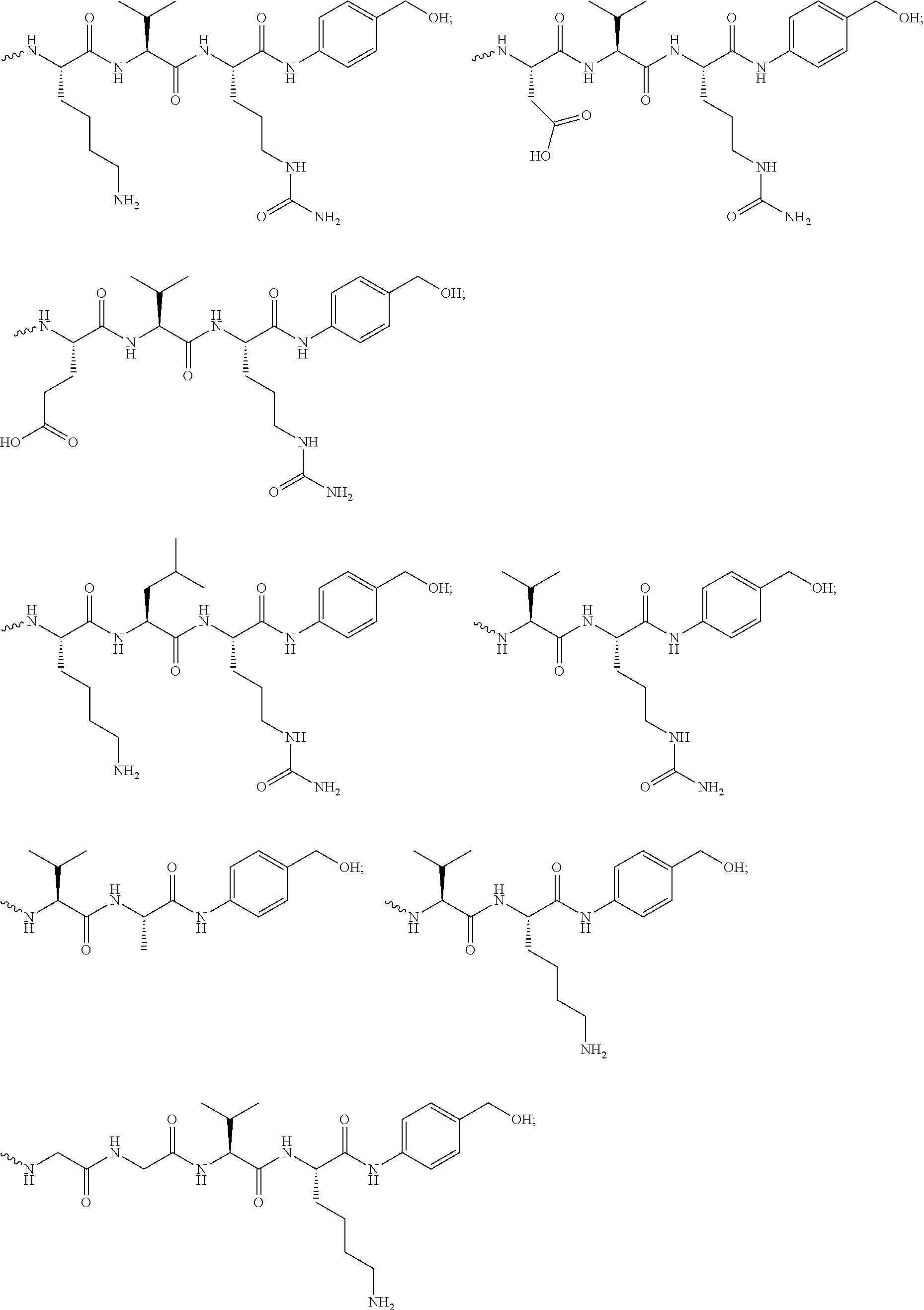

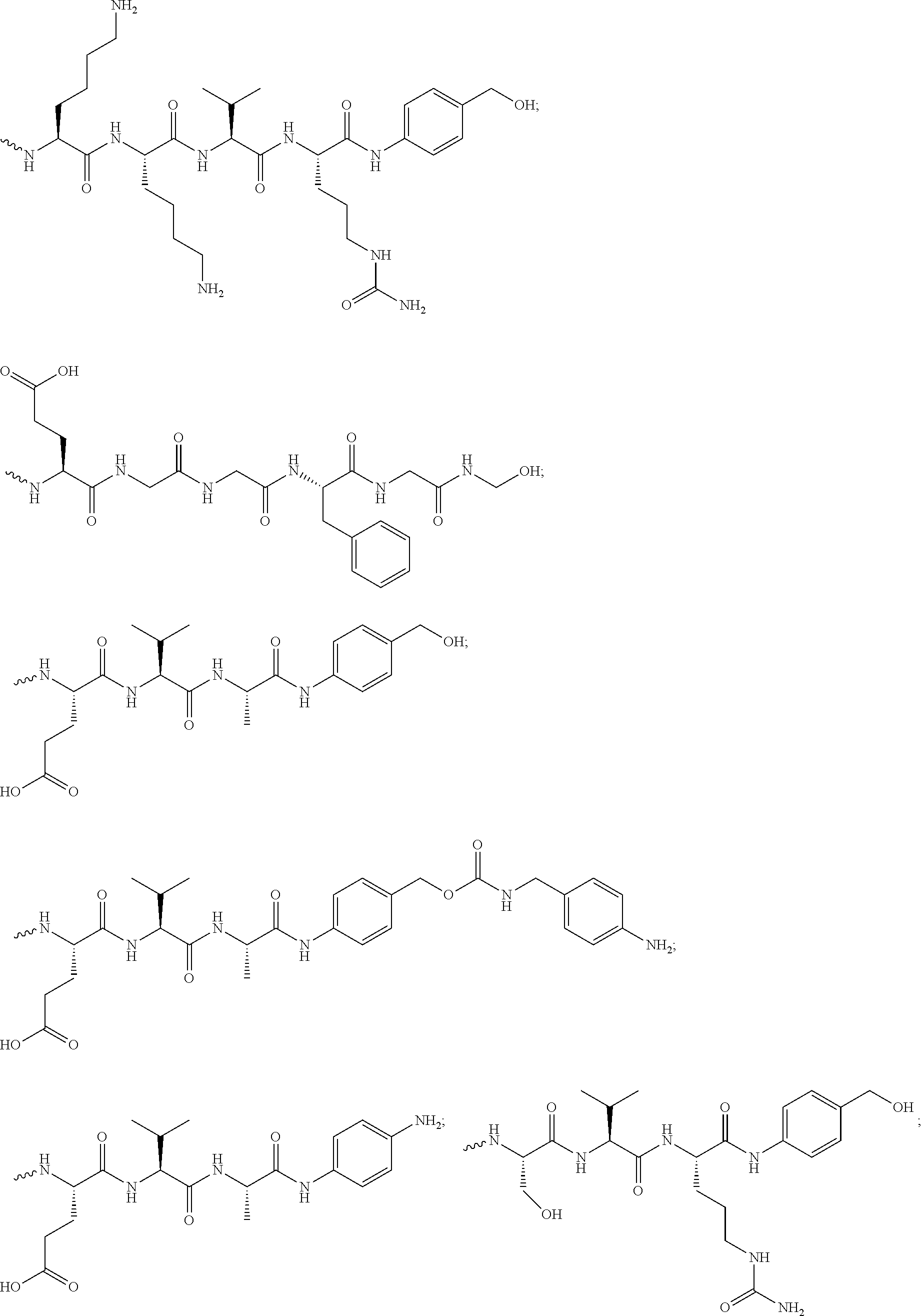

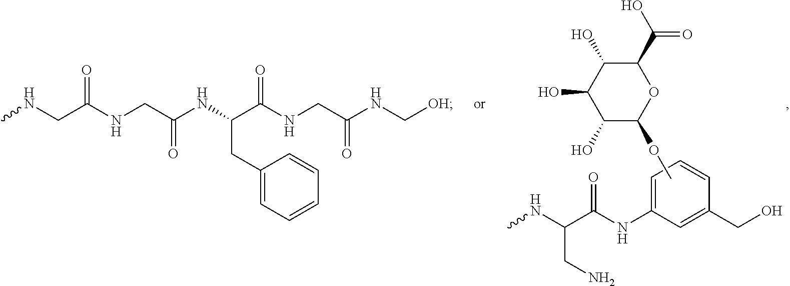

or a stereoisomer thereof, wherein the Polar group is attached to an amino acid subunit of the Amino Acid unit, the H of a hydroxyl or amino group of the para-aminobenzyl group or the H of a hydroxyl of the glycine residue of a GGFG peptide is optionally replaced with a bond to at least one of the Drug units, or to a linking group attached to the at least one of the Drug units, the wavy line on the amino group indicates an attachment site for a Stretcher unit or an Amino Acid unit or, prior to attachment, indicates H. In some embodiments, at least one of the Drug units is attached directly to the benzylic oxygen (—O—). In some embodiments, at least one of the Drug units is attached indirectly, via a linking group. A linking group can be any suitable group for connection of the at least one Drug unit to the benzylic —O— that allows for release of an active Drug unit, or release of an active derivative of the linking group-Drug unit. In some embodiments, a linking group is —NH—CH₂—CH₂—CH₂—C(O)—, the Drug unit is exatecan and the released Drug unit is DXd. (See., e.g., Published US Application No. 2019/000898.) In other embodiments, the linking group is —C(O)—NH—CH₂—CH₂—CH₂—C(O)—.

In some embodiments, provided is a Linker compound, comprising a formula selected from the following:

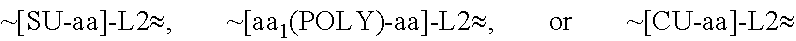

wherein the square brackets indicate the Amino Acid unit, each aa is an optional subunit of the Amino Acid unit, L2 is the Linker unit, each wavy line (˜) indicates an attachment site for a Stretcher unit; aa₁(POLY) is a Polymer unit attached to an amino acid subunit of the Amino Acid unit, SU is a Sugar unit attached to a subunit of the Amino Acid unit or to the Linker unit, and CU is a Carboxyl unit attached to a subunit of the Amino Acid unit or to the Linker unit; and the double wavy (≈) line indicates an attachment site for at least one of the Drug units, wherein aa and aa₁ are independently selected from alpha, beta and gamma amino acids and derivatives thereof.

In some embodiments, provided is a Linker compound, comprising a formula selected from the following:

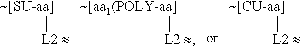

wherein the square brackets indicate the Amino Acid unit, each aa is an amino acid subunit of the Amino Acid unit, L2 is the Linker Subunit attached to a side chain of aa, the wavy line (˜) indicates an attachment site for a Stretcher unit; aa₁(POLY) is a Polymer unit attached to aa, SU is a Sugar unit attached to aa, CU is a Carboxyl unit attached to aa, and the double wavy (≈) line indicates an attachment site for at least one of the Drug units; wherein aa and aa₁ are independently selected from alpha, beta and gamma amino acids and derivatives thereof.

In some embodiments, provided is a Linker compound, wherein at least two Polymer units are attached to the Amino Acid unit.

In some embodiments, provided is a Linker compound, comprising a formula selected from the following:

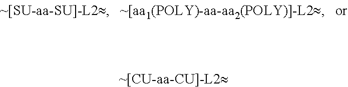

wherein the square brackets indicate the Amino Acid unit, aa is an optional subunit of the Amino Acid unit, L2 is the Linker unit, the wavy line (˜) indicates an attachment site for a Stretcher unit; each of aa₁(POLY) and aa₂(POLY) is a Polymer unit attached to aa or to the other Polymer unit; each SU is a Sugar unit attached to aa or the other Sugar unit, each CU is a Carboxyl unit attached to aa or to the other Carboxyl unit, and the double wavy (≈) line indicates an attachment site for at least one of the Drug units; wherein aa, aa₁ and aa₂ are independently selected from alpha, beta and gamma amino acids and derivatives thereof.

In some embodiments, provided is a Linker compound, comprising a formula selected from the following:

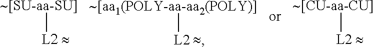

wherein the square brackets indicate the Amino Acid unit, aa is an amino acid subunit of the Amino Acid unit, L2 is a Linker unit attached to a side chain of aa, each wavy line (˜) indicates an attachment site for a Stretcher unit; each of aa₁(POLY) and aa₂(POLY) is a Polymer unit attached to aa, each SU is a Sugar unit attached to aa; each CU is a Carboxyl unit attached to aa; and the double wavy (≈) line indicates an attachment site for at least one of the Drug units; wherein each of aa, aa₁ and aa₂ is independently selected from alpha, beta and gamma amino acids and derivatives thereof.

In some embodiments, provided is a Linker compound, wherein the Linker Unit is a cleavable linker unit.

In some embodiments, provided is a Linker compound, wherein the Linker Unit comprises a peptide that is cleavable by an intracellular protease.

In some embodiments, the intracellular protease is Cathepsin B.

In some embodiments, provided is a Linker compound, wherein the cleavable peptide comprises a valine-citrulline peptide, a valine-alanine peptide, a valine-lysine peptide, a phenylalanine-lysine peptide, or a glycine-glycine-phenylalanine-glycine peptide.

In some embodiments, provided is a Linker compound, wherein the Amino Acid unit comprises a peptide that is cleavable by an intracellular protease.

In some embodiments, provided is a Linker compound, wherein the cleavable peptide comprises a valine-citrulline peptide, a valine-alanine peptide, a valine-lysine peptide, a phenylalanine-lysine peptide, or a glycine-glycine-phenylalanine-glycine peptide.

In some embodiments, provided is a Linker compound, wherein the cleavable peptide is attached to a para-aminobenzyl alcohol self immolative group (PABA).

In some embodiments, provided is a Linker compound, comprising one of the following structures:

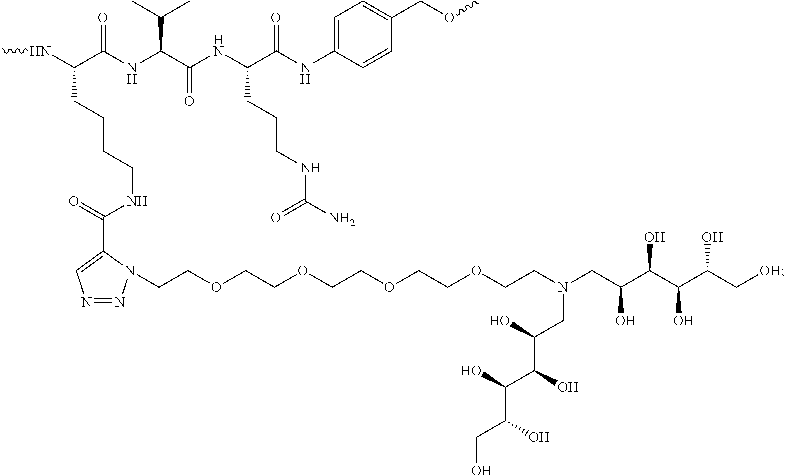

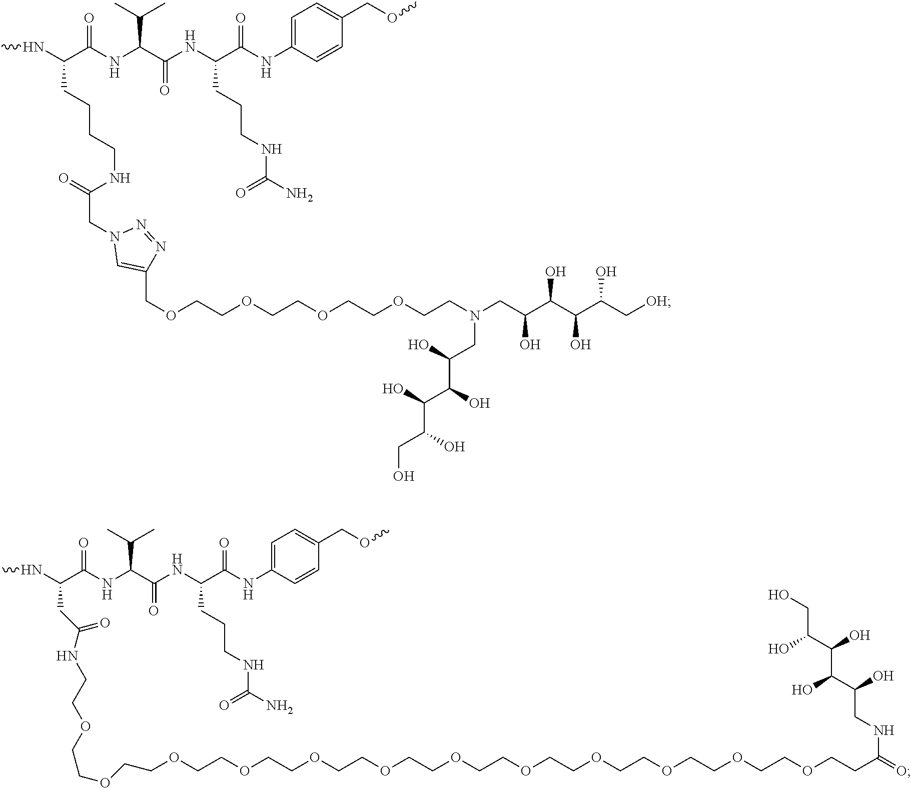

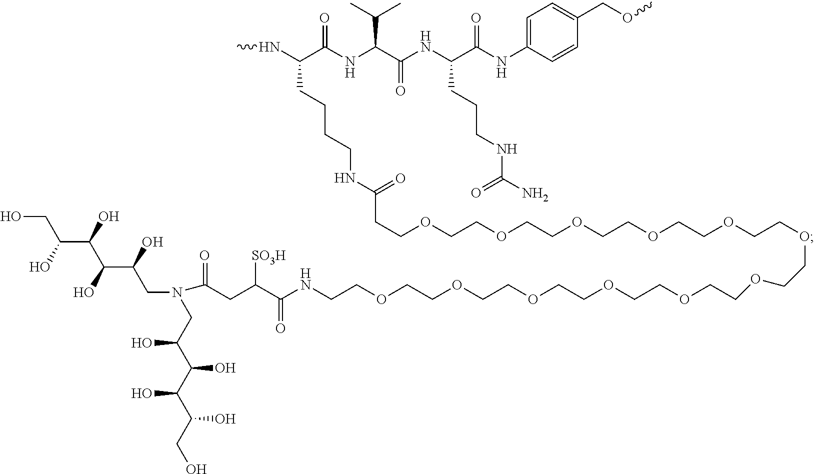

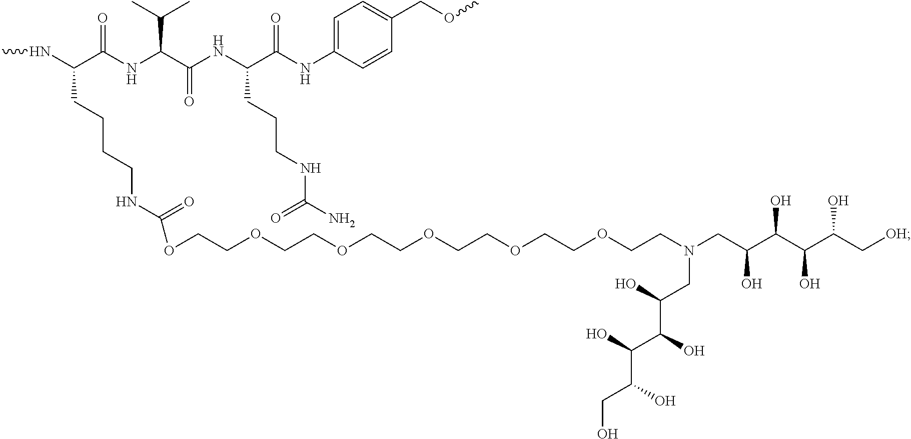

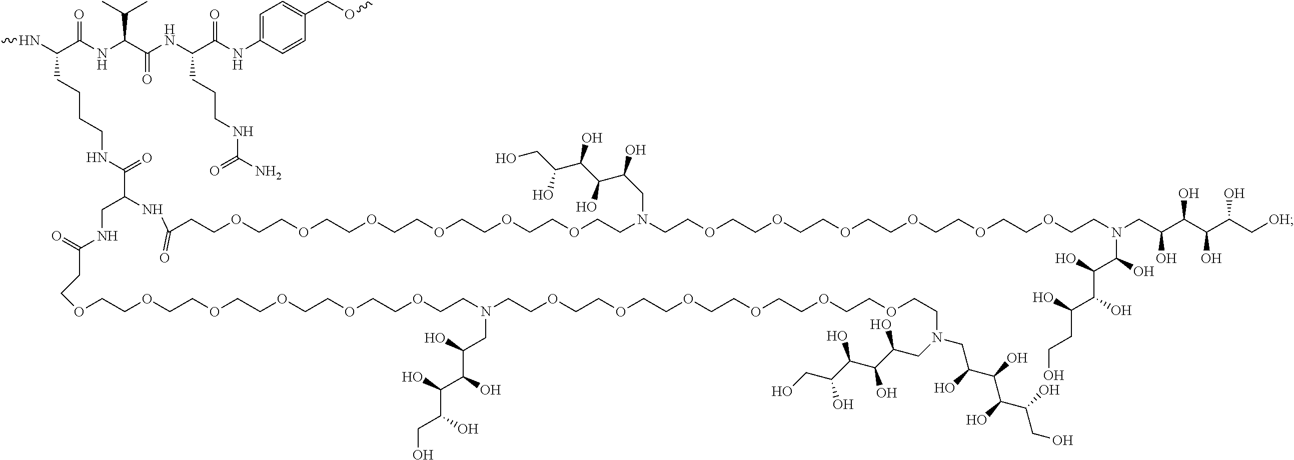

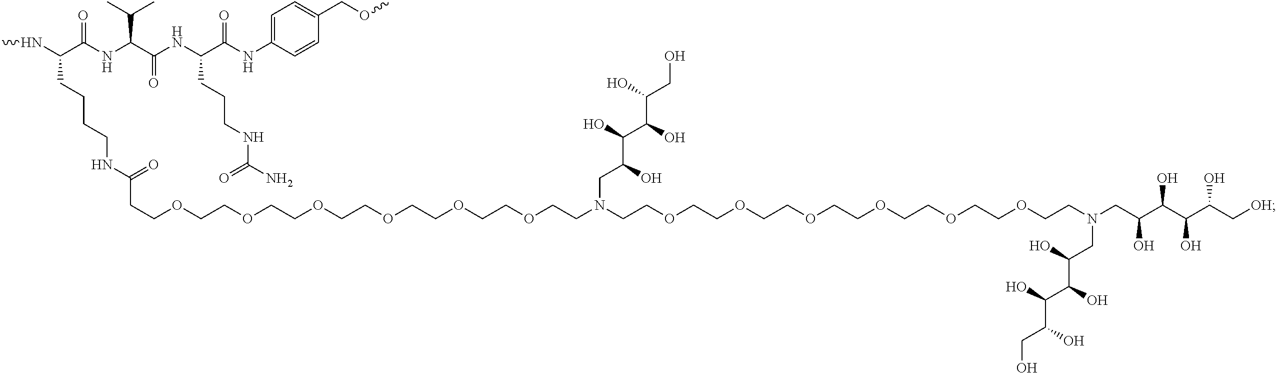

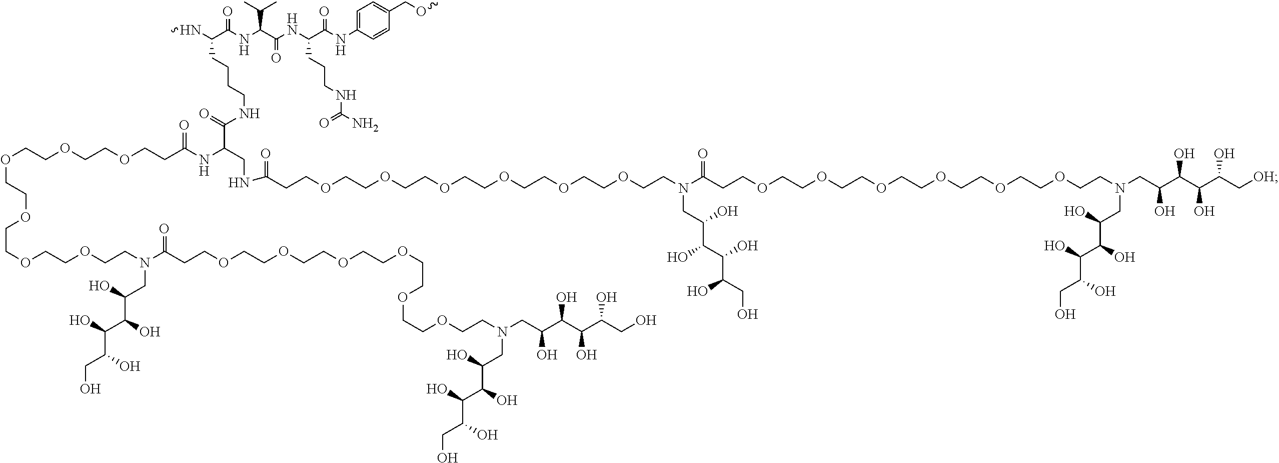

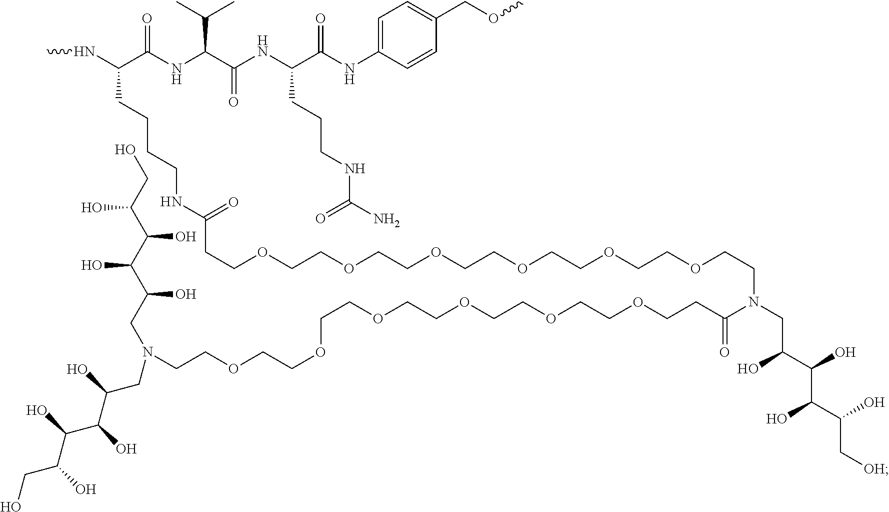

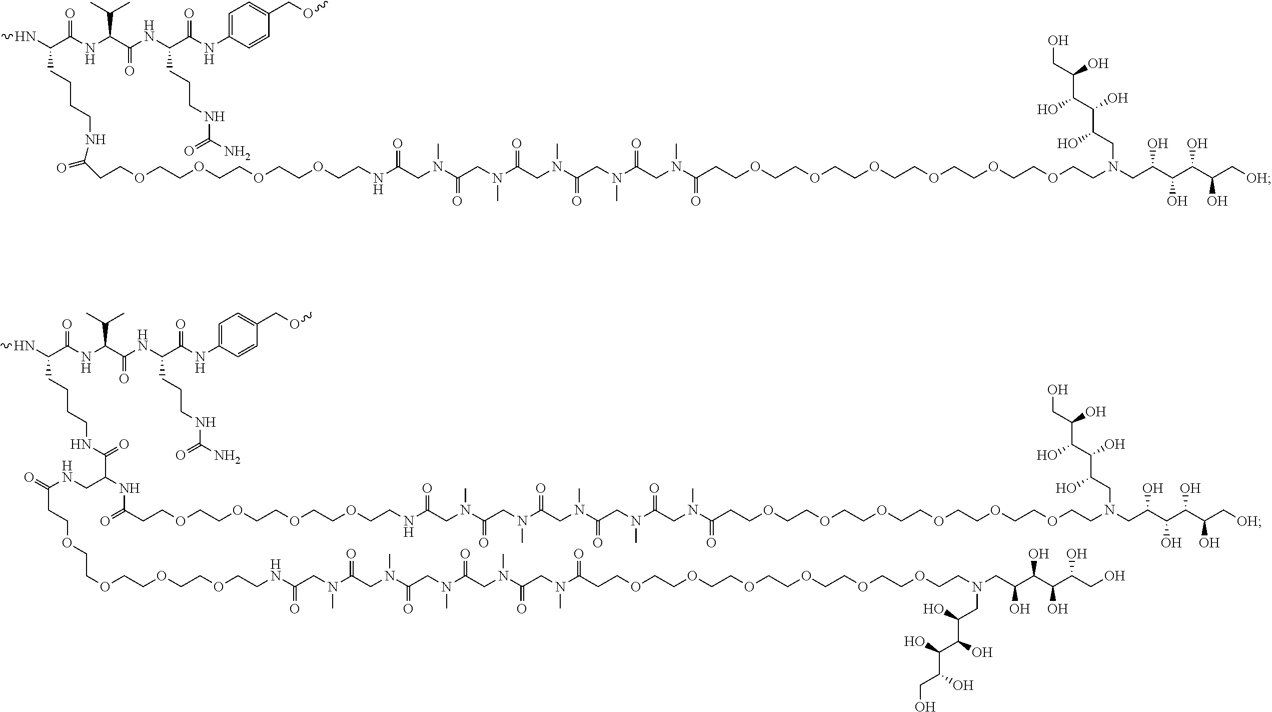

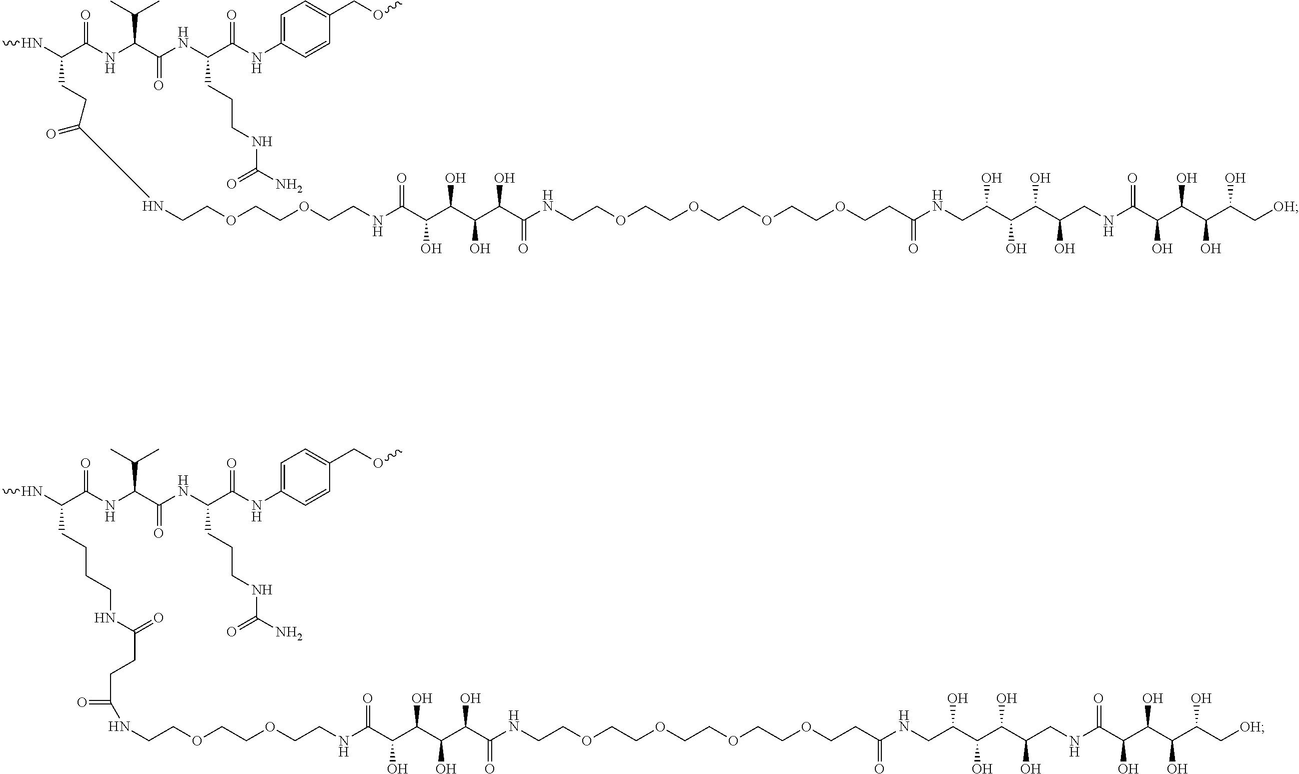

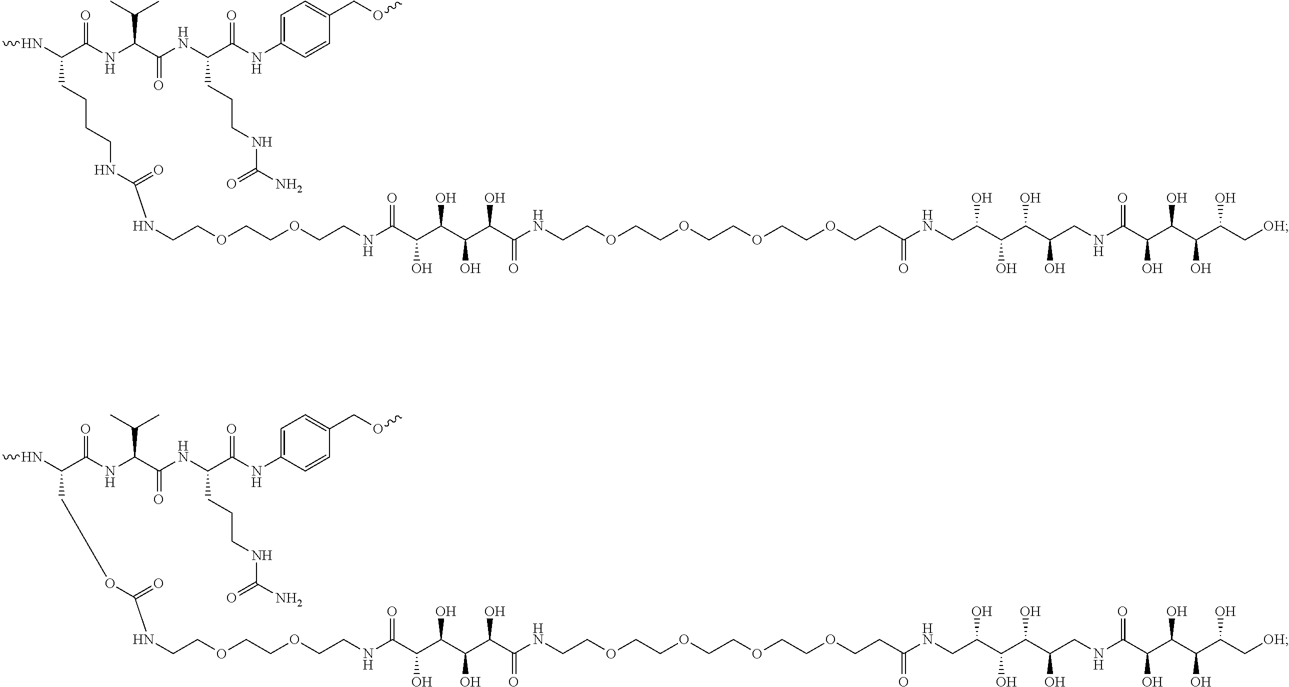

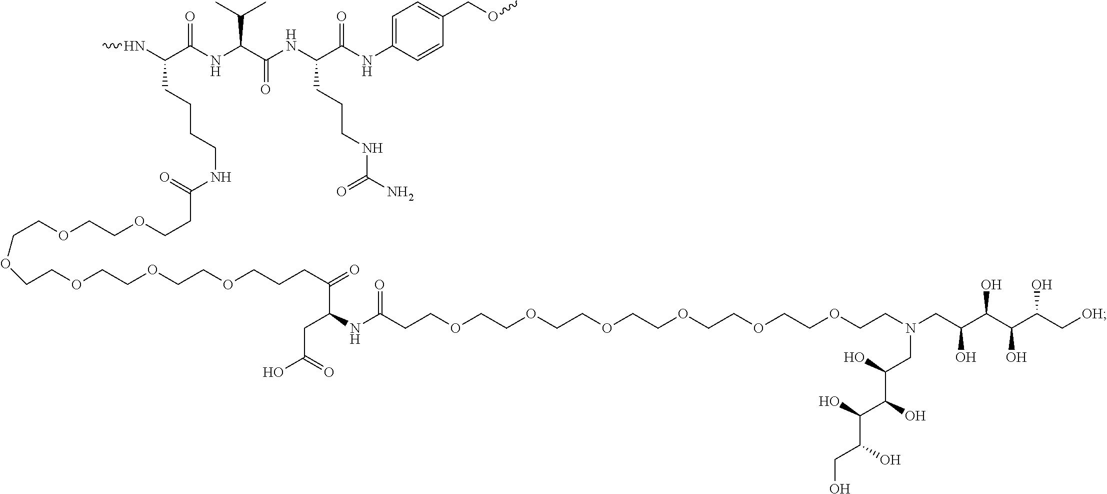

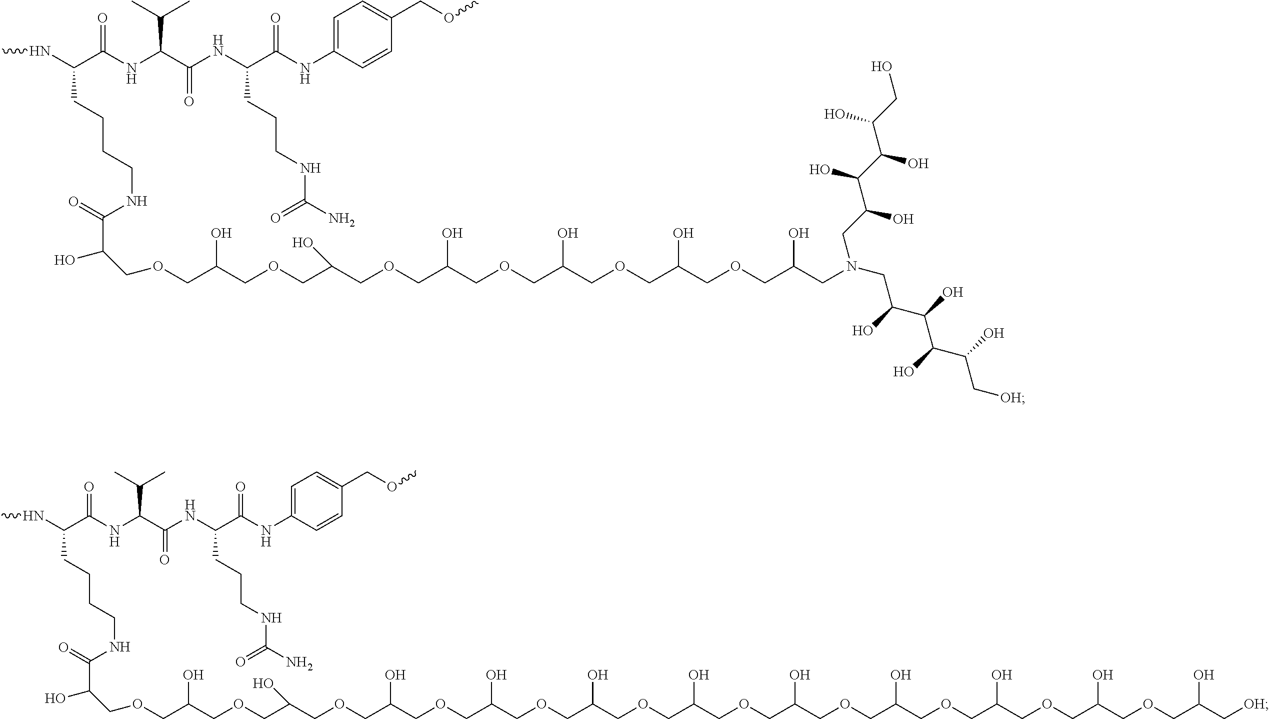

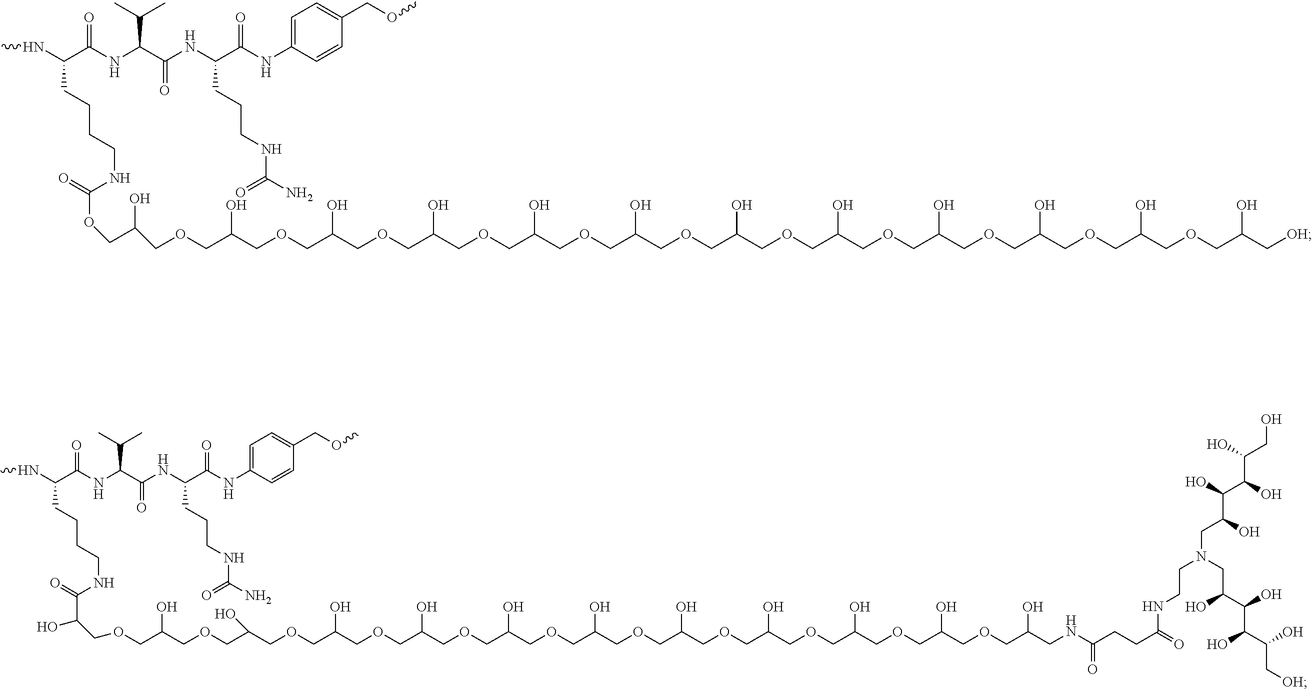

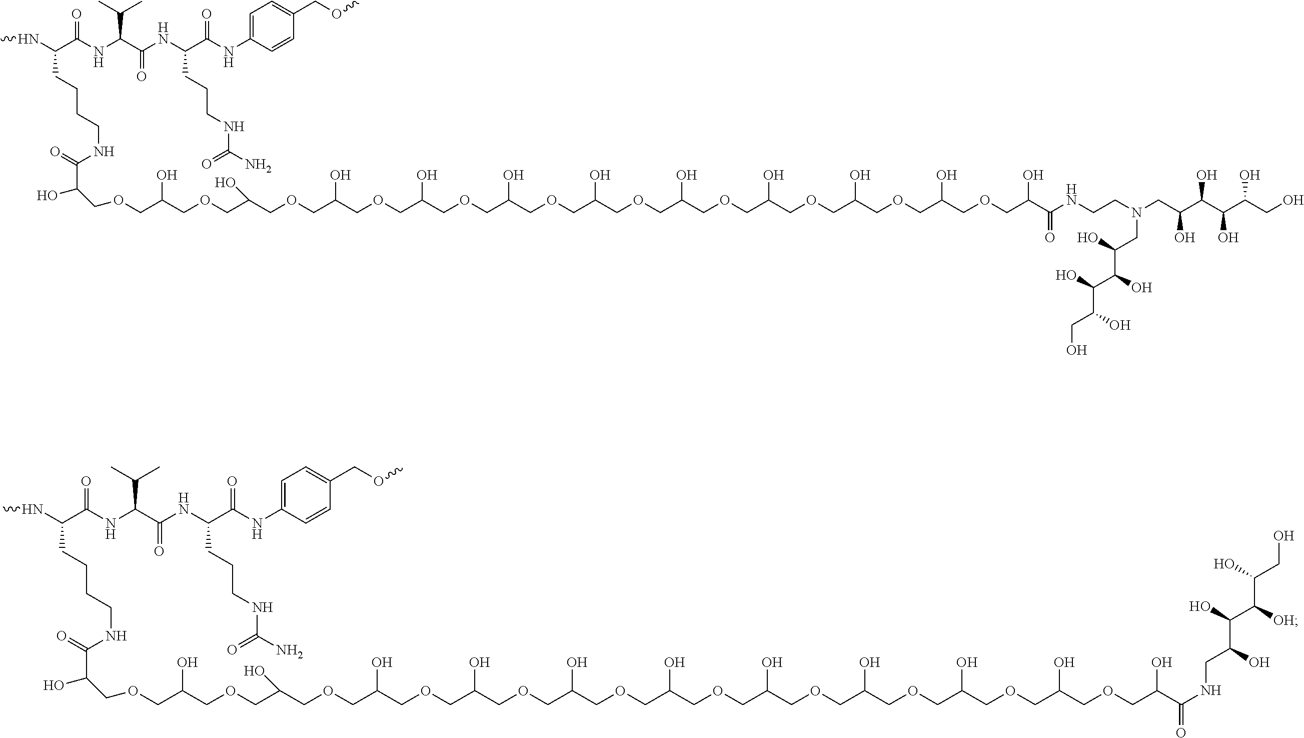

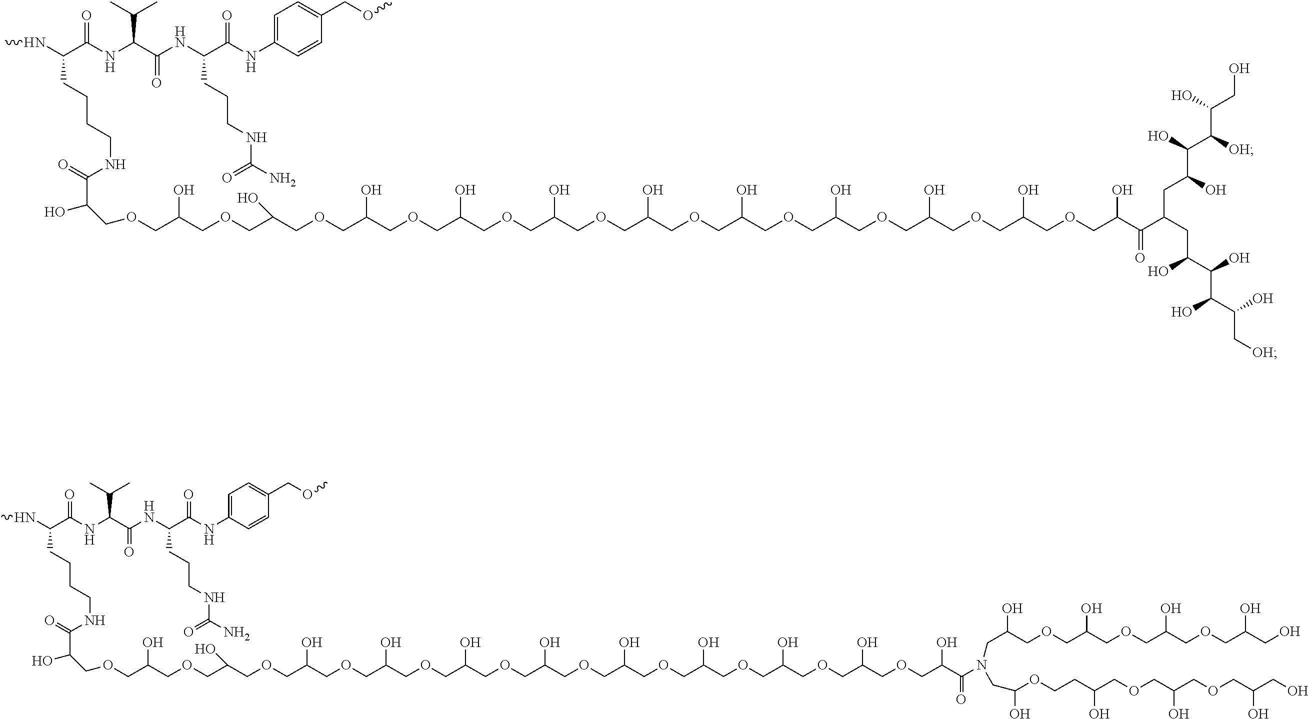

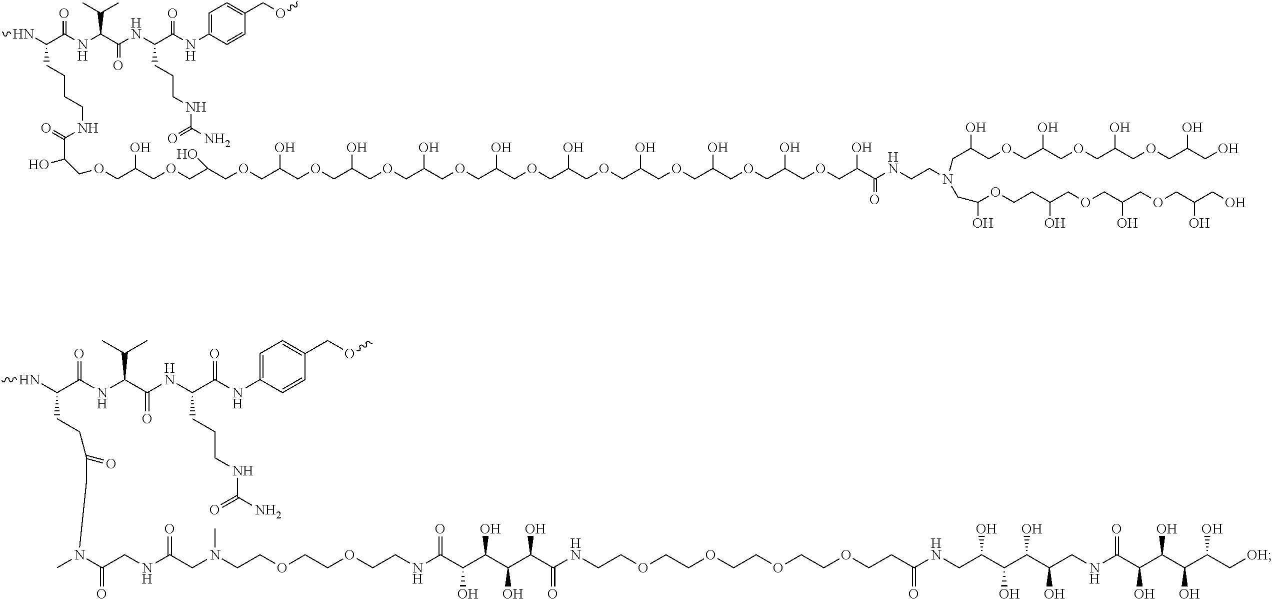

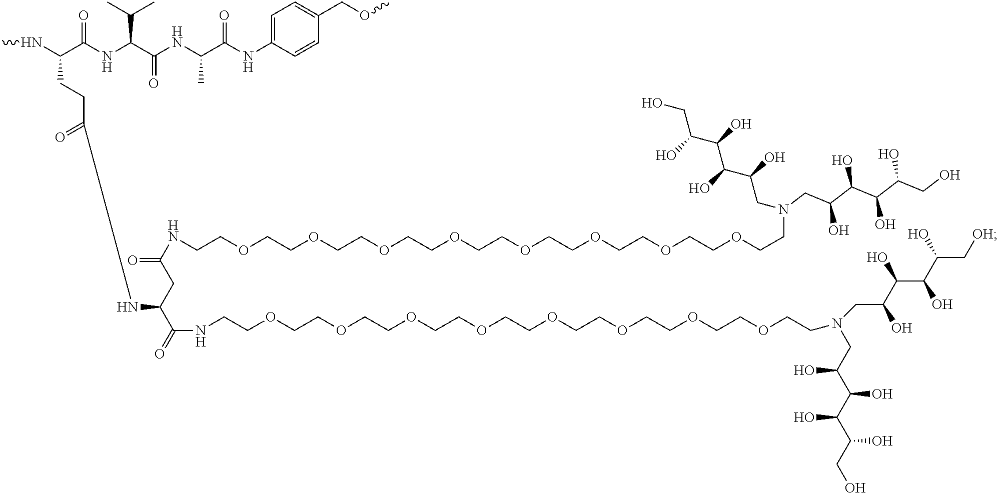

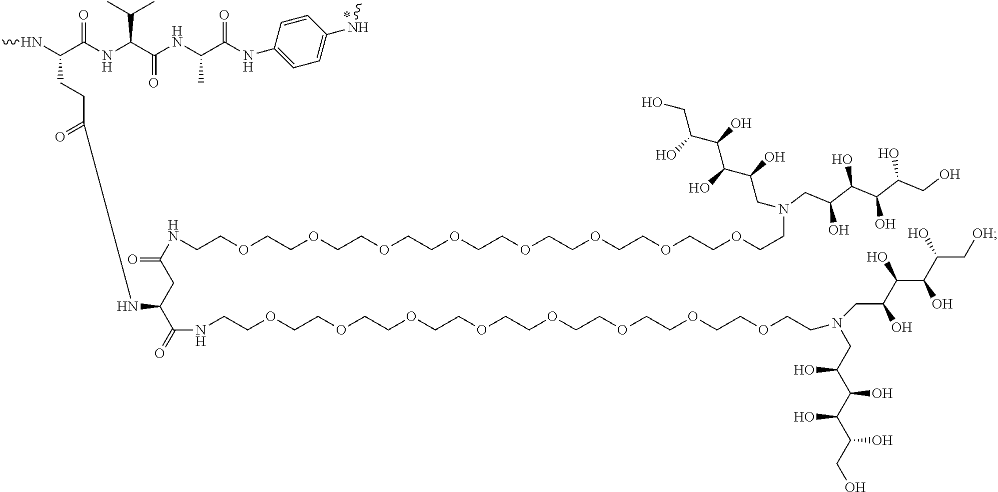

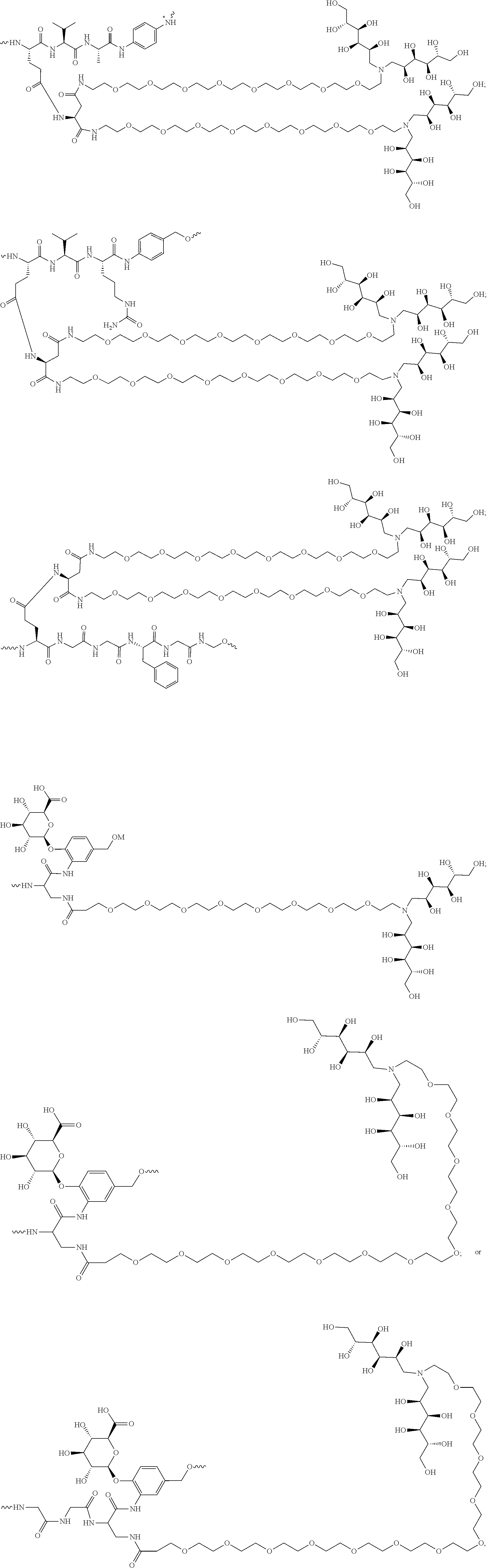

wherein the wavy line [symbol] on the oxygen group or the *-amino group indicates the attachment site for at least one of the Drug units or for a linking group attached to the at least one of the Drug units; and the wavy line [symbol] von the amino group indicates an attachment site for a Stretcher unit or an Amino Acid unit or, prior to attachment, indicates H.

In some embodiments, at least one of the Drug units is attached directly to the benzylic O. In some embodiments, at least one of the Drug units is attached indirectly, via a linking group. A linking group can be any suitable group for connection of the at least one Drug unit to the benzylic oxygen (—O—) that allows for release of an active Drug unit, or release of an active derivative of the linking group-Drug unit. In some embodiments, a linking group is —NH—CH₂—CH₂—CH₂—C(O)—, the Drug unit is exatecan and the released Drug unit is DXd. (See., e.g., Published US Application No. 2019/000898.)

In some embodiments, provided is a Linker compound, wherein the Linker unit further comprises a Stretcher unit having an attachment site for a Targeting unit and wherein the Stretcher unit is attached to the Amino Acid unit of the Linker compound.

In some embodiments, provided is a Linker compound, wherein the Stretcher unit is selected from the following:

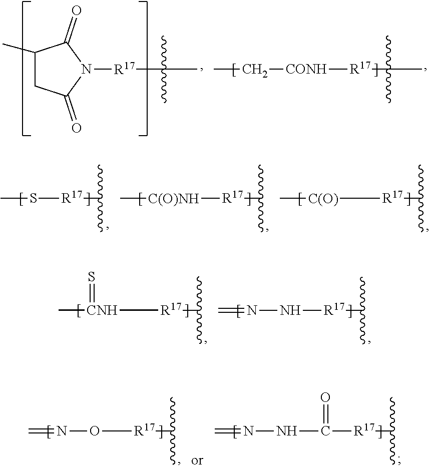

wherein each ( [symbol] ) indicates an attachment site to an Amino Acid unit;
wherein R¹⁷ is —C₁-C₁₀ alkylene-, —C₁-C₁₀ heteroalkylene-, —C₃-C₈ carbocyclo-, —O—(C₁-C₈ alkylene)-, —(CH₂—O—CH₂)_{b}—C₁-C₈ alkylene- (where b is 1 to 26), —C₁-C₈ alkylene-(CH₂—O—CH₂)_{b}— (where b is 1 to 26), —C₁-C₈ alkylene-(CH₂—O—CH₂)_{b}—C₁-C₈ alkylene- (where b is 1 to 26), -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₁-C₈ alkylene-(CH₂—O—CH₂)_{b}—C(═O)— (where b is 1 to 26), —(CH₂—O—CH₂)_{b}—C₁-C₈ alkylene-C(═O)— (where b is 1 to 26), —C₁-C₈ alkylene-(CH₂—O—CH₂)_{b}—C₁-C₈ alkylene-C(═O)— (where b is 1 to 26), —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₁-C₈ alkylene-(CH₂—O—CH₂)_{b}—NH— (where b is 1 to 26), —(CH₂—O—CH₂)_{b}—C₁-C₈ alkylene-NH— (where b is 1 to 26), —C₁-C₈ alkylene-(CH₂—O—CH₂)_{b}—C₁-C₈ alkylene-NH— (where b is 1 to 26), —C₁-C₈ alkylene-(C(═O))—NH—(CH₂—O—CH₂)_{b}—C(═O)— (where b is 1 to 26), —C₁-C₈ alkylene-(C(═O))—NH—(CH₂—O—CH₂)_{b}—C₁-C₈ alkylene-C(═O)— (where b is 1 to 26), —C₁-C₈ alkylene-NH—(C(═O))—(CH₂—O—CH₂)_{b}—NH— (where b is 1 to 26), —C₁-C₈ alkylene-NH—(C(═O))—(CH₂—O—CH₂)_{b}—C₁-C₈ alkylene-NH— (where b is 1 to 26), —C₃-C₈ carbocyclo-NH—, —O—(C₁-C₈ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkyl)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—; or
wherein the Stretcher unit comprises maleimido(C₁-C₁₀alkylene-C(O)—, maleimido(CH₂OCH₂)ₚ₂(C₁-C₁₀alkylene)C(O)—, maleimido(C₁-C₁₀alkylene)(CH₂OCH₂)ₚ₂C(O)—, or a ring open form thereof, wherein p2 is from 1 to 26.

In some embodiments, provided is a Linker compound, wherein the Stretcher unit is selected from the following:

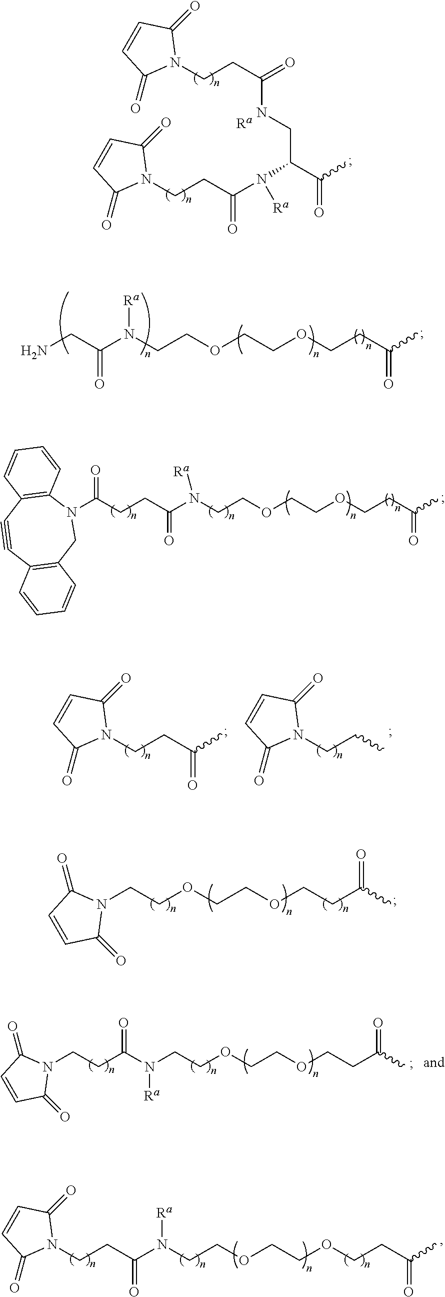

or a stereoisomer thereof, wherein each R^{a} is independently H or C₁₋₆ alkyl, each n is
   independently 0-12, and the wavy line [symbol] indicates an attachment site of the Stretcher unit to the Amino Acid unit, and the attachment site for the Targeting unit is on a maleimide, primary amine or alkyne functional group.

In some embodiments, provided is a Linker compound, wherein the Stretcher unit is selected from the following:

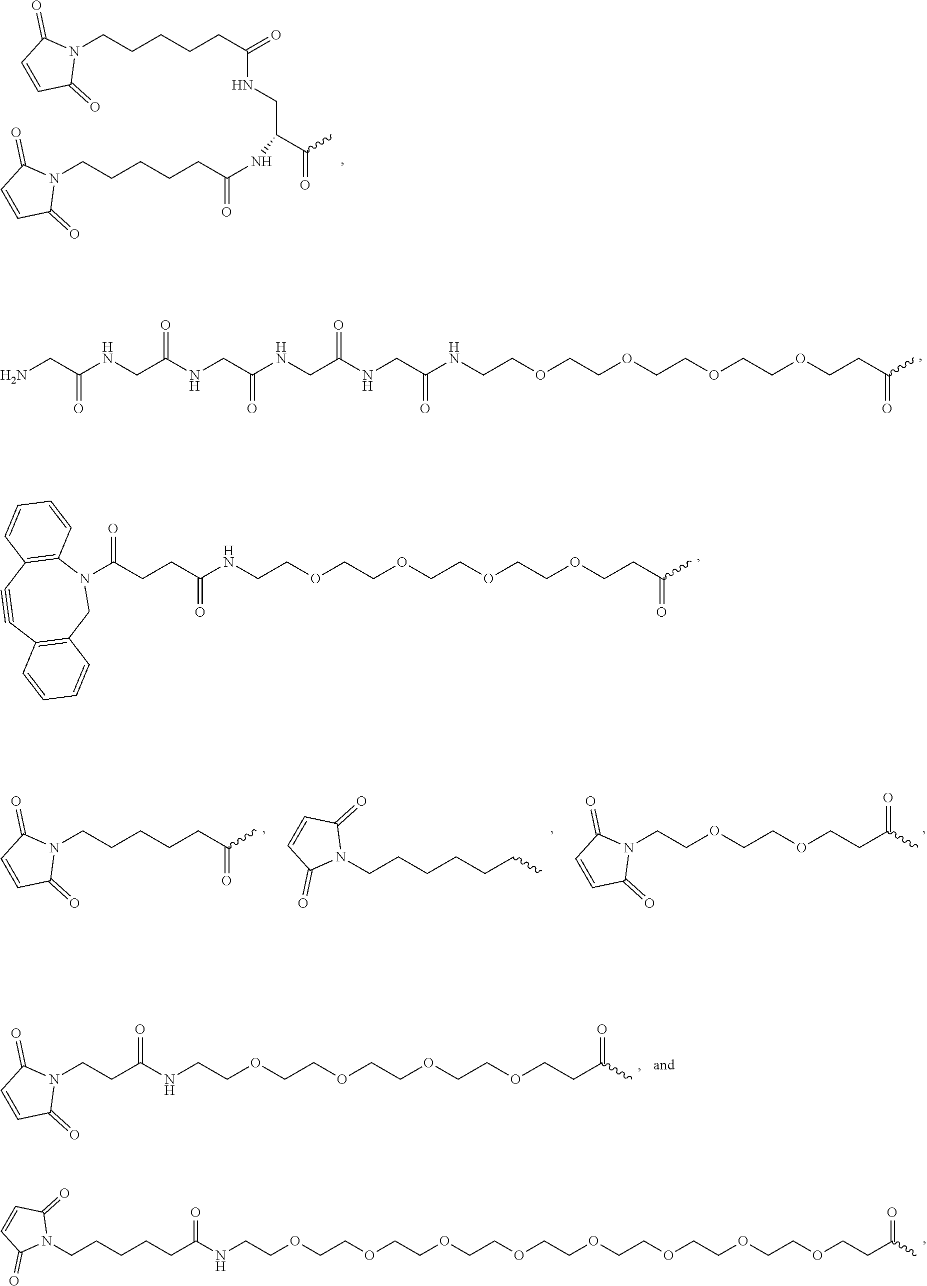

or a stereoisomer thereof, wherein the wavy line [symbol] indicates an attachment site of the Stretcher unit to an Amino Acid unit, and the attachment site for the Targeting unit is on a maleimide, primary amine or alkyne functional group.

In some embodiments, provided is a Linker compound, comprising one of the following structures:

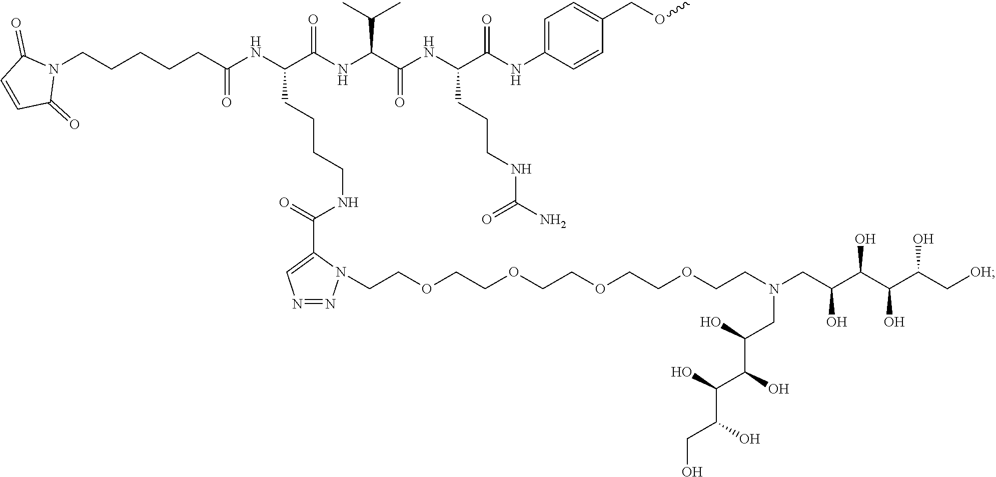

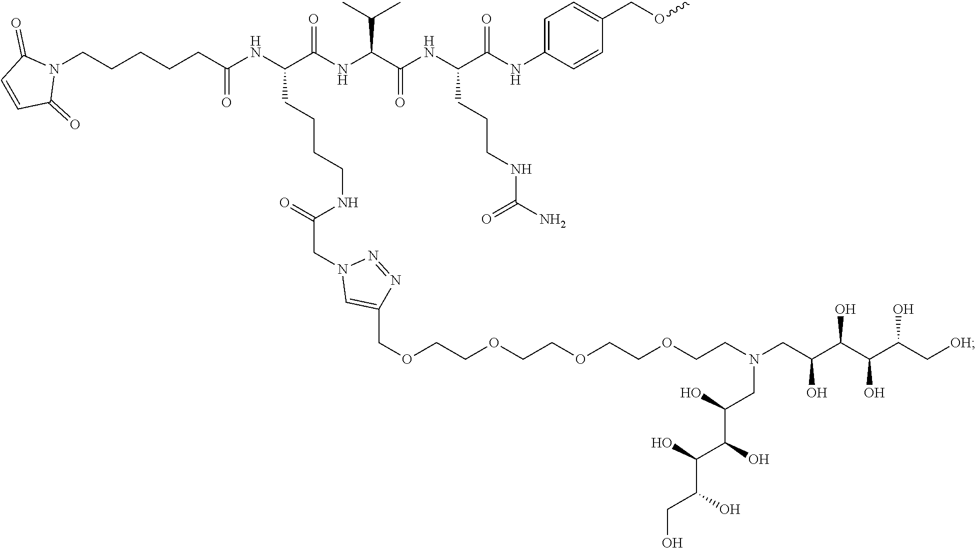

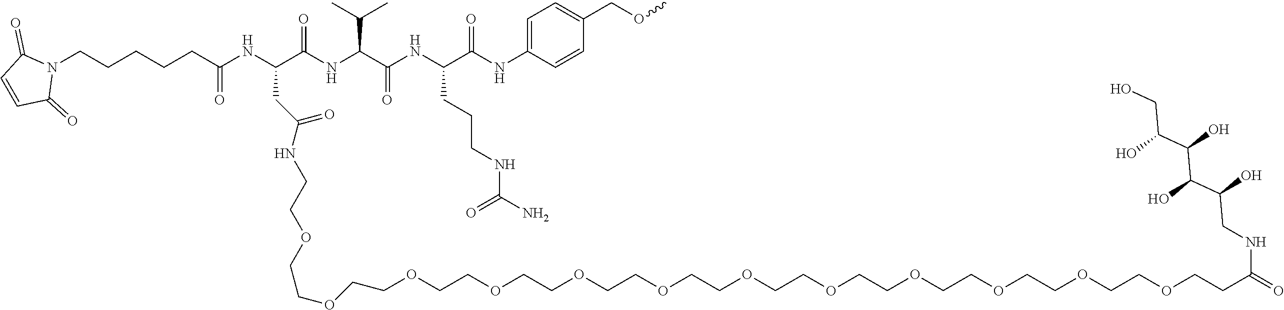

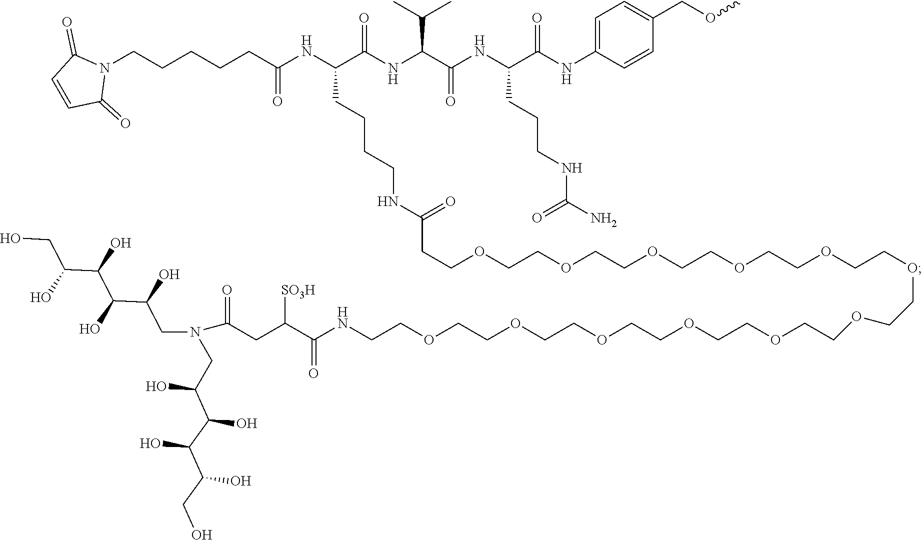

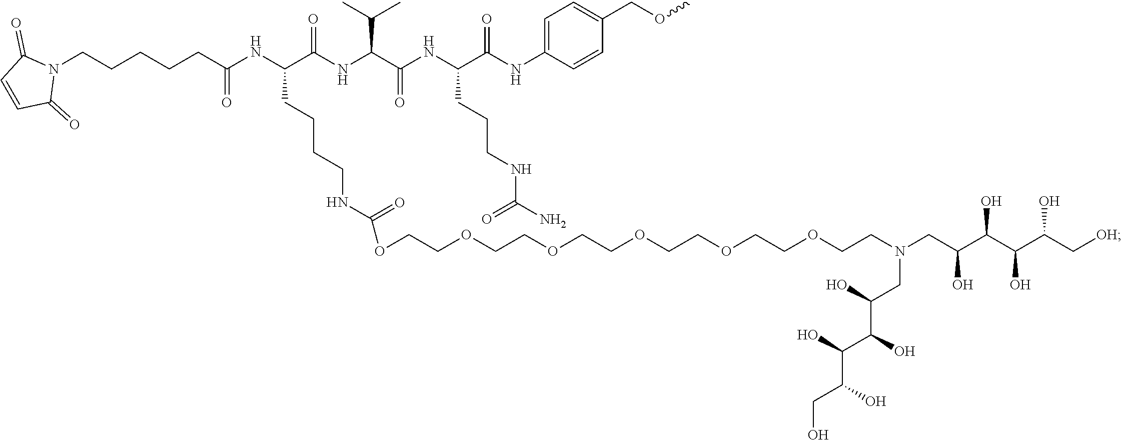

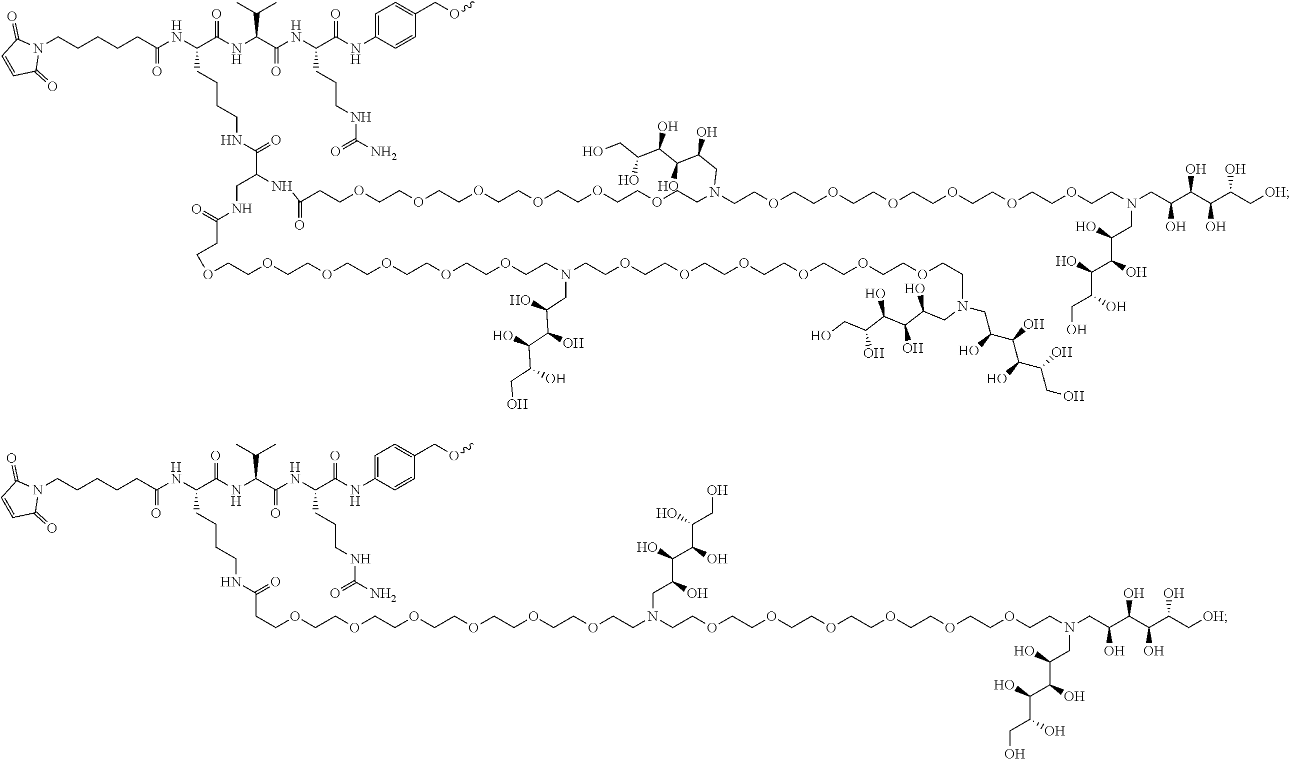

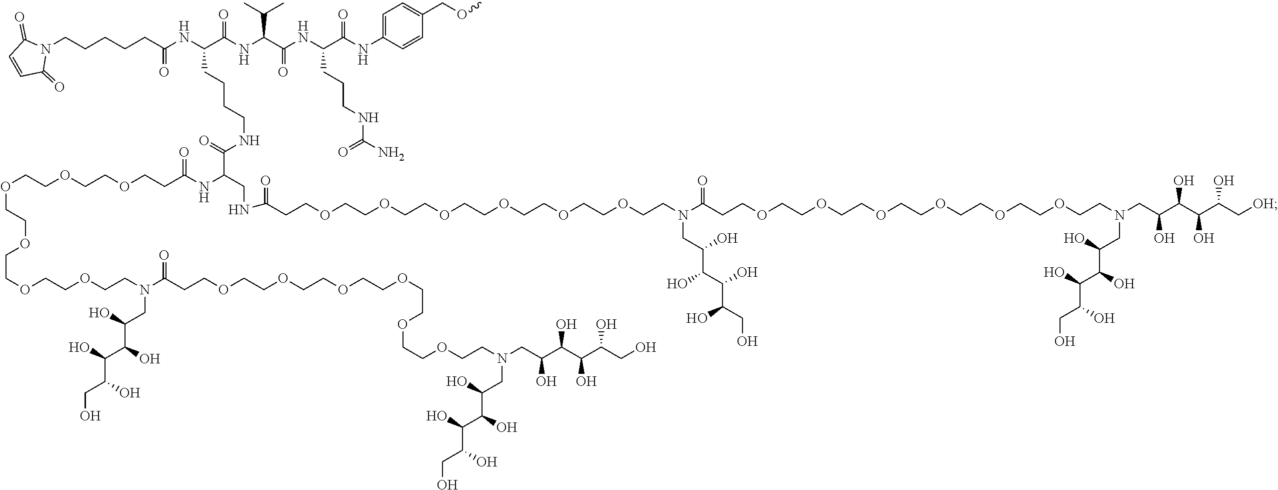

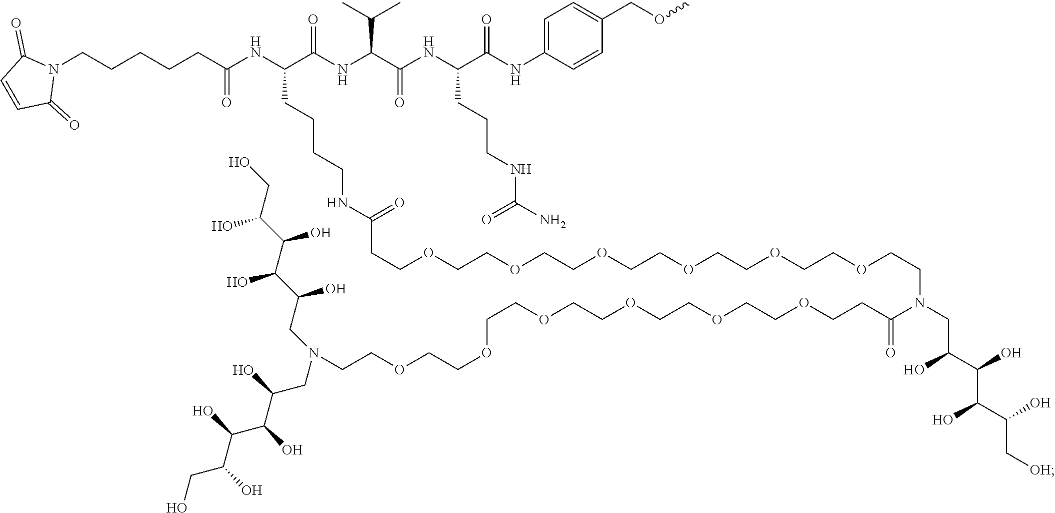

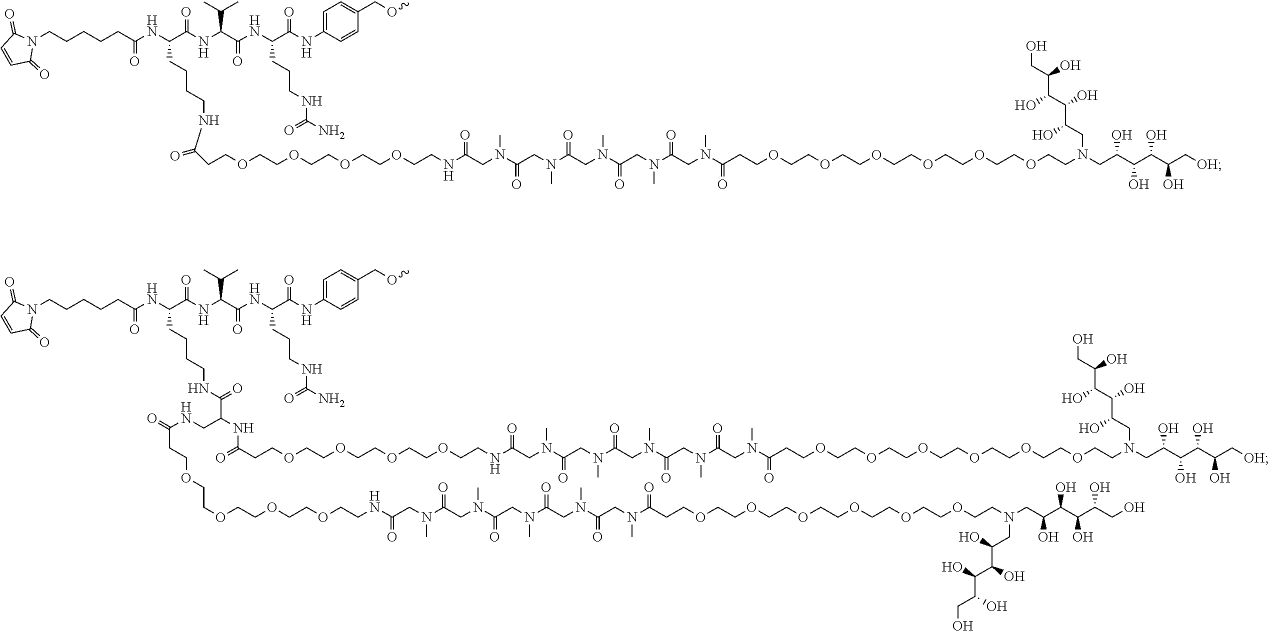

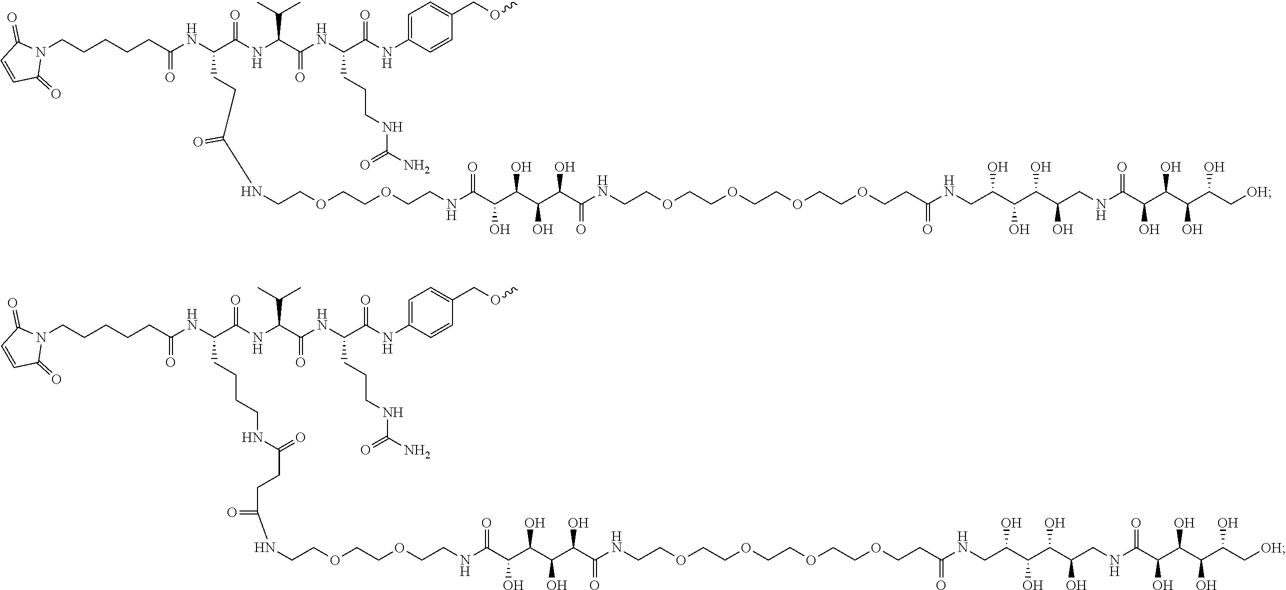

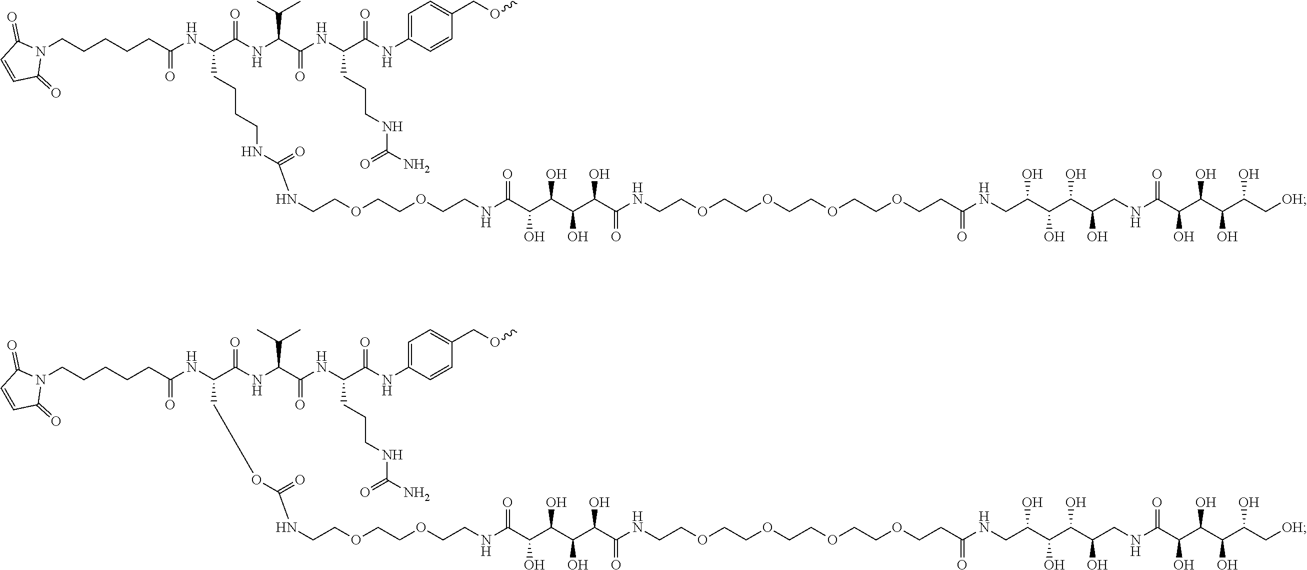

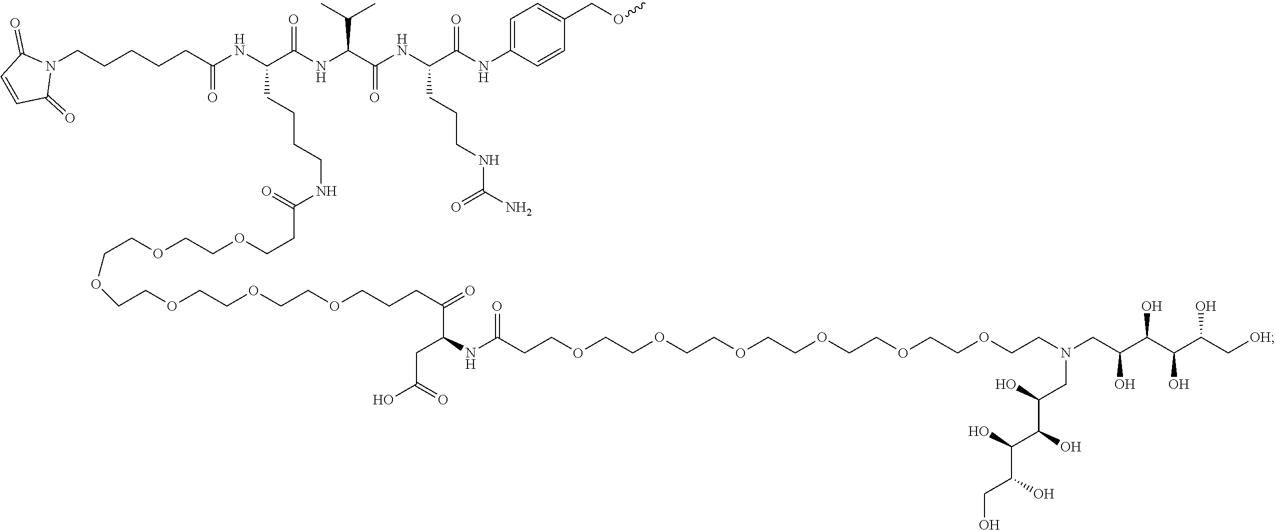

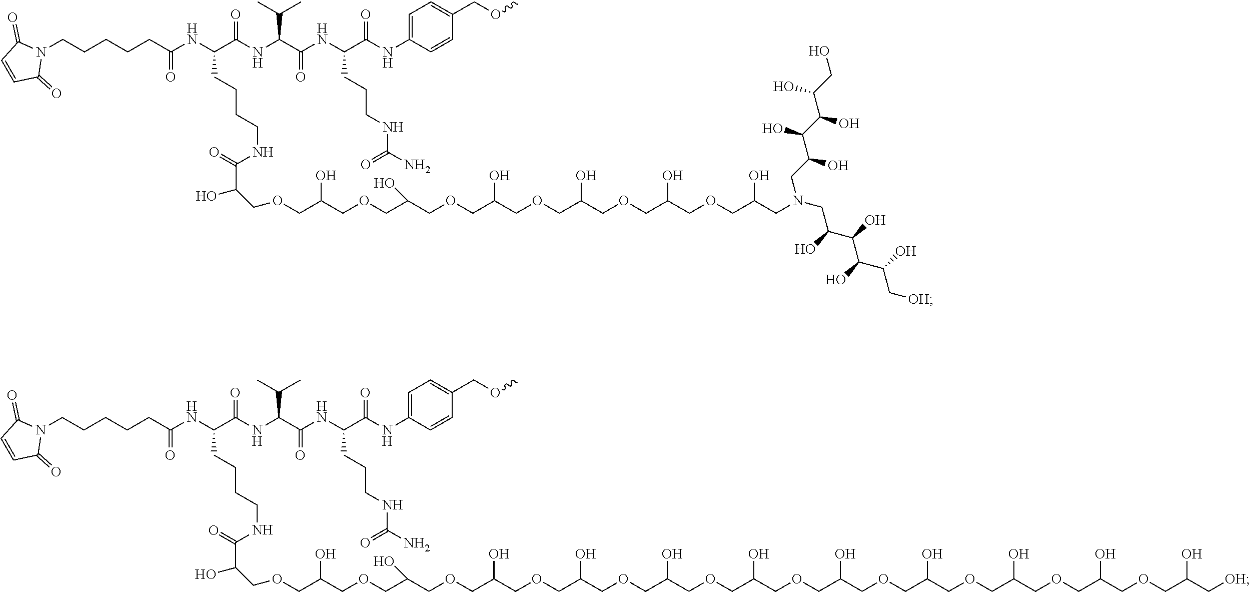

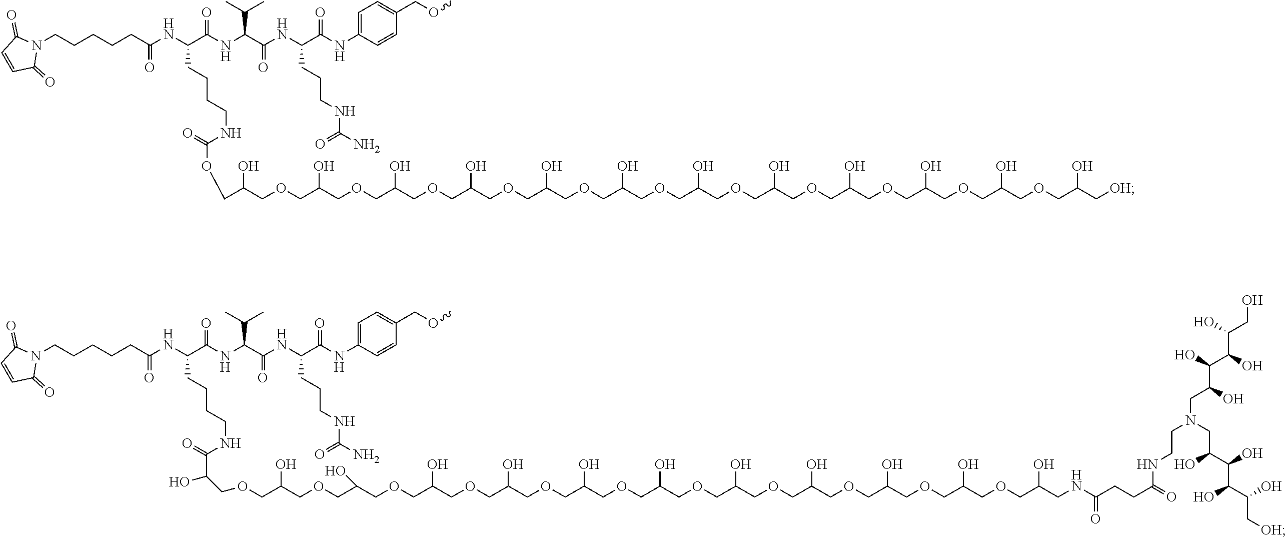

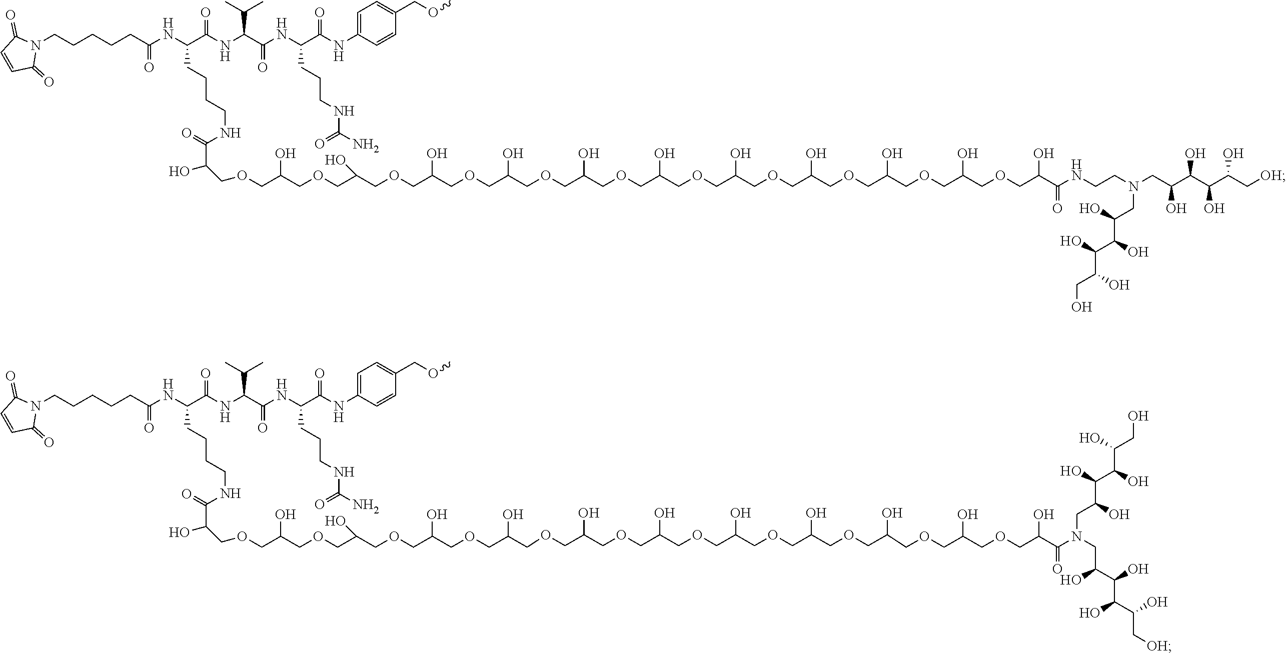

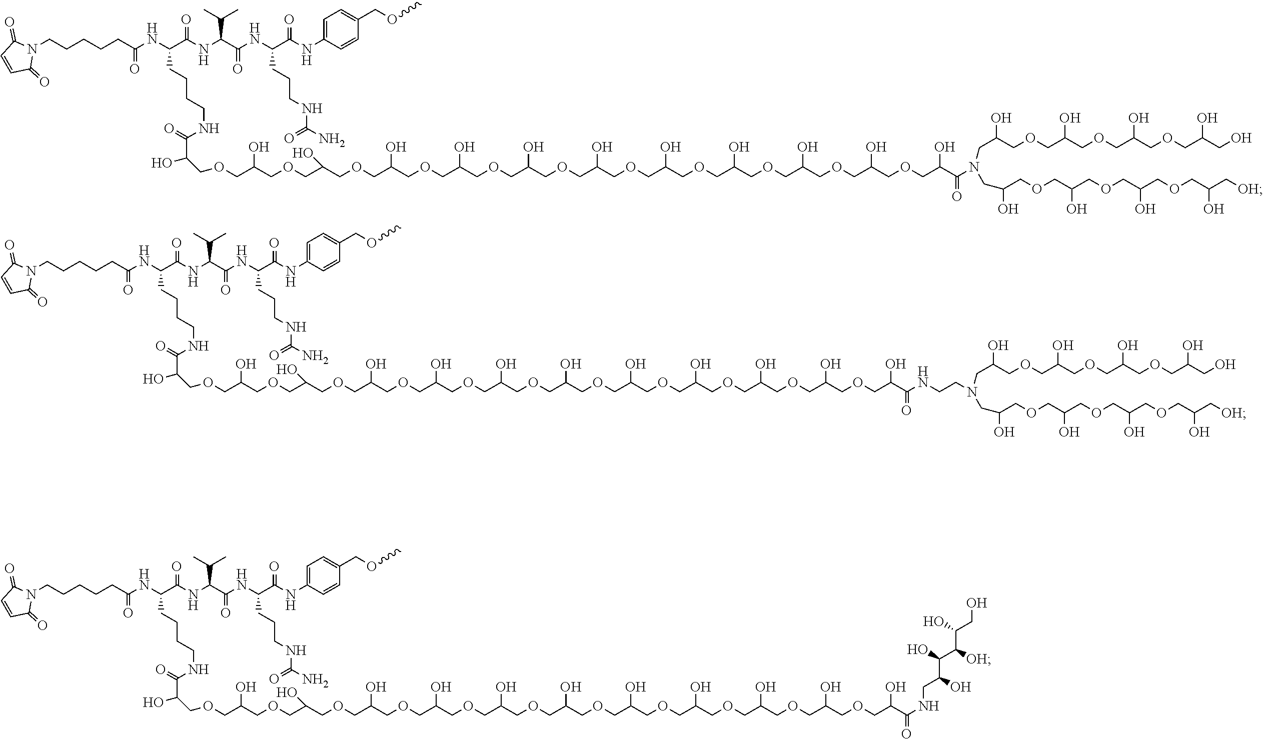

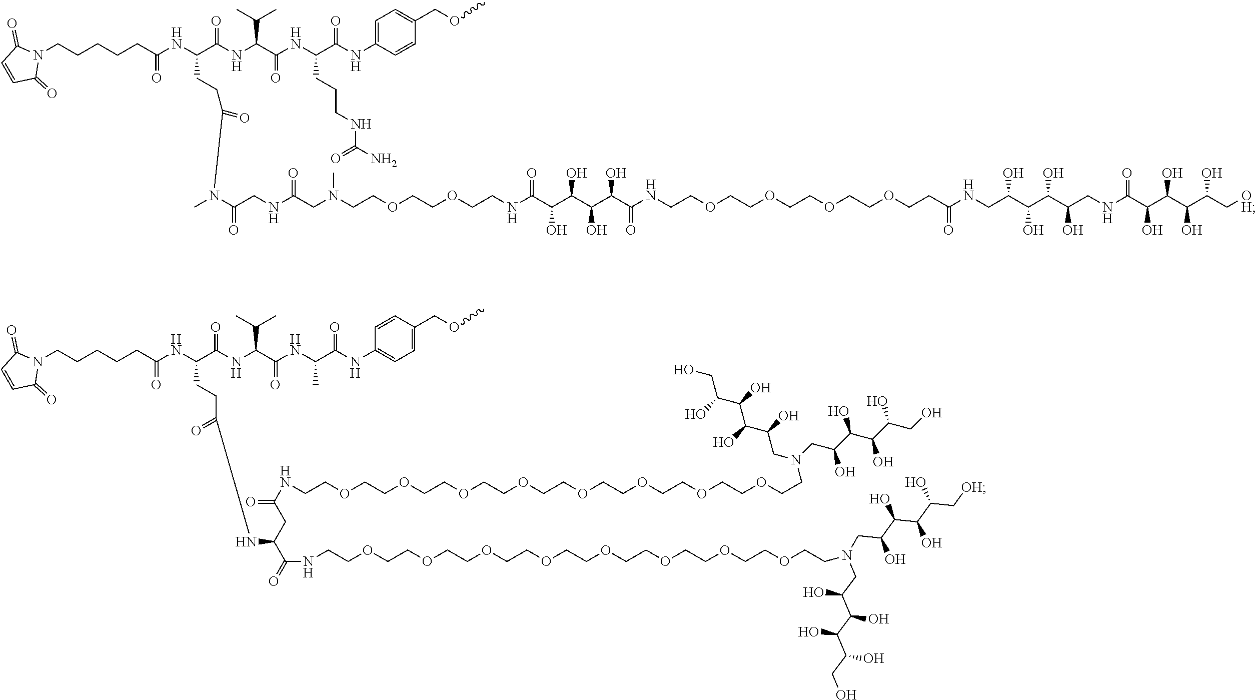

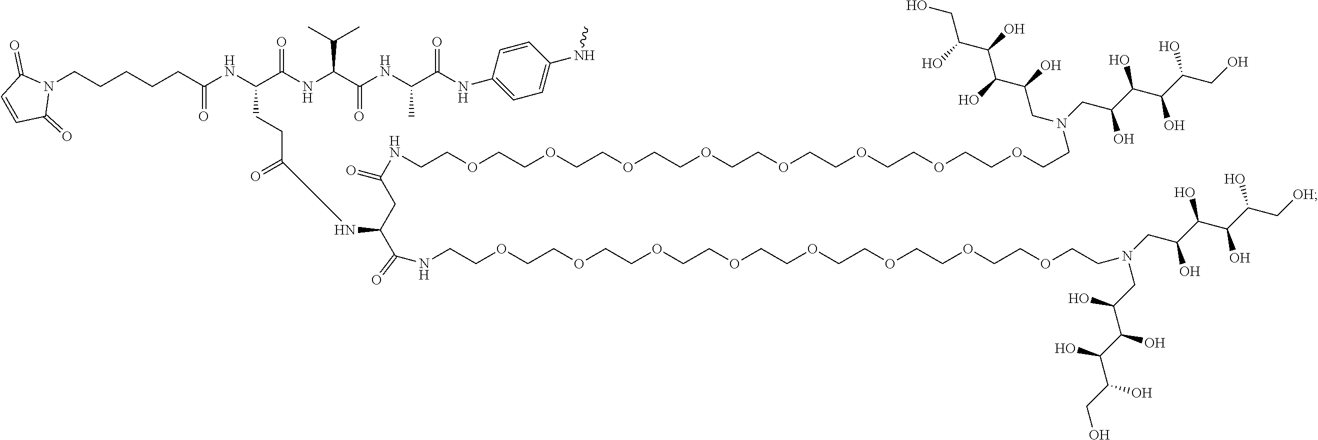

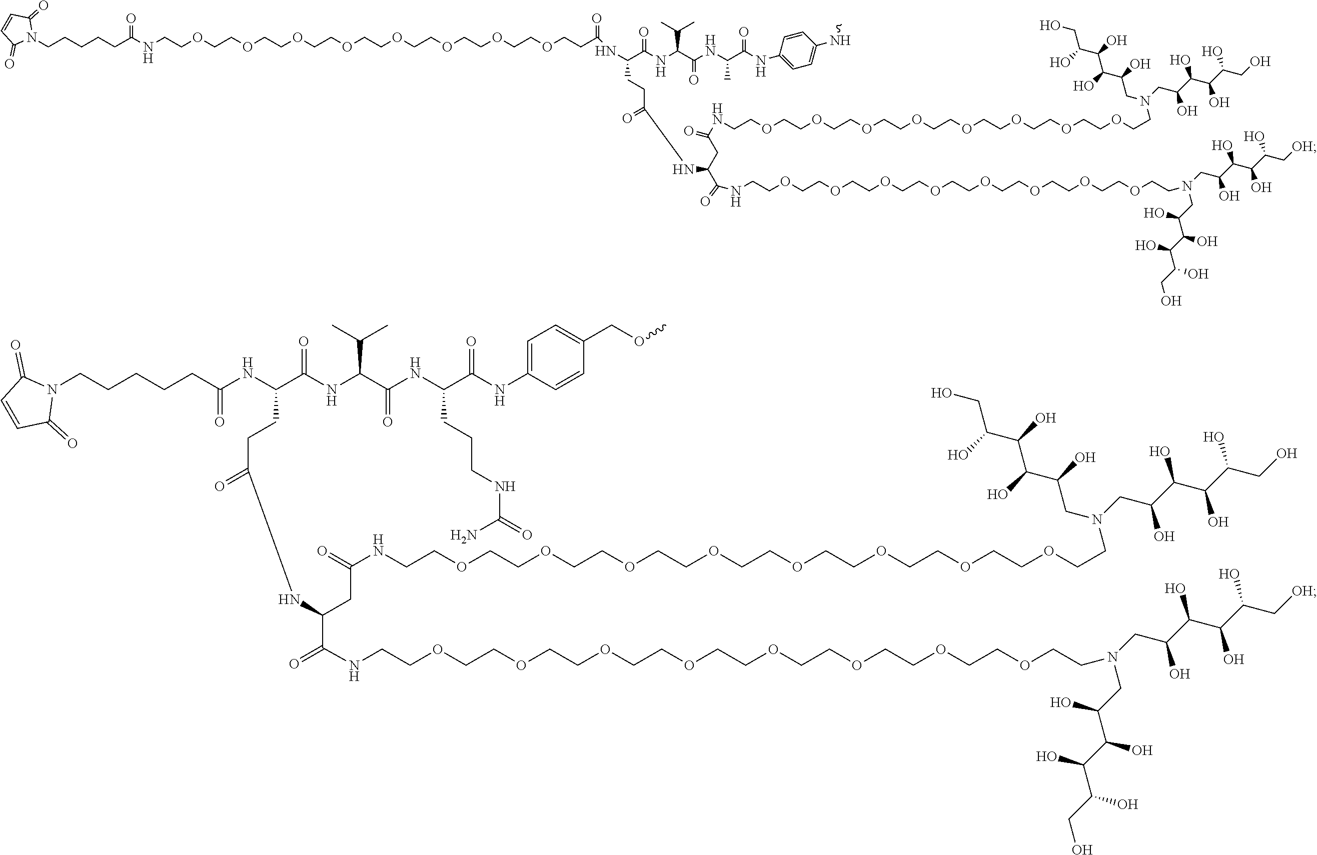

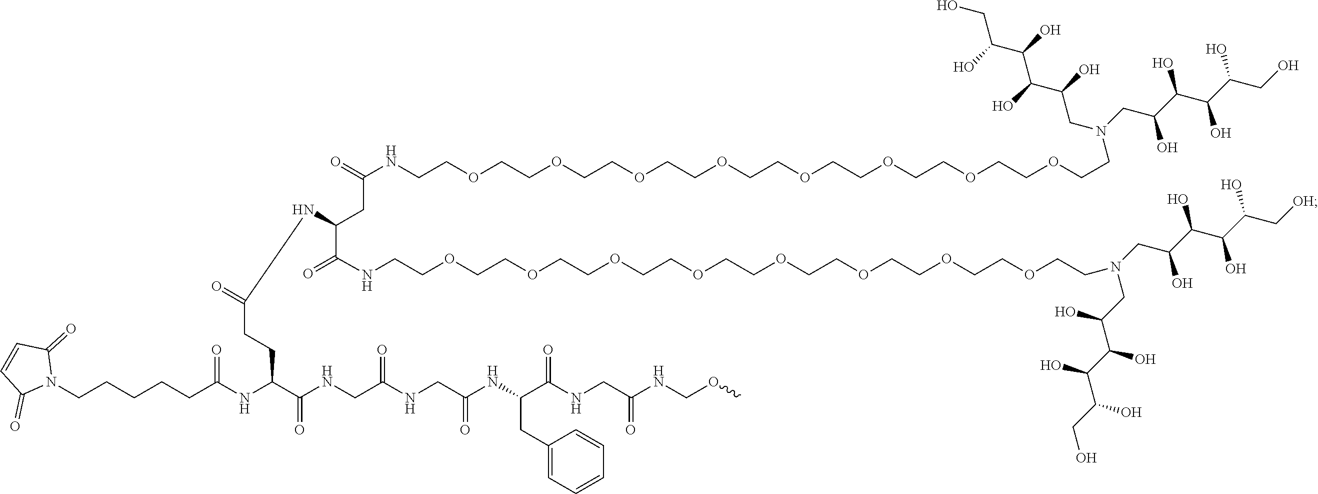

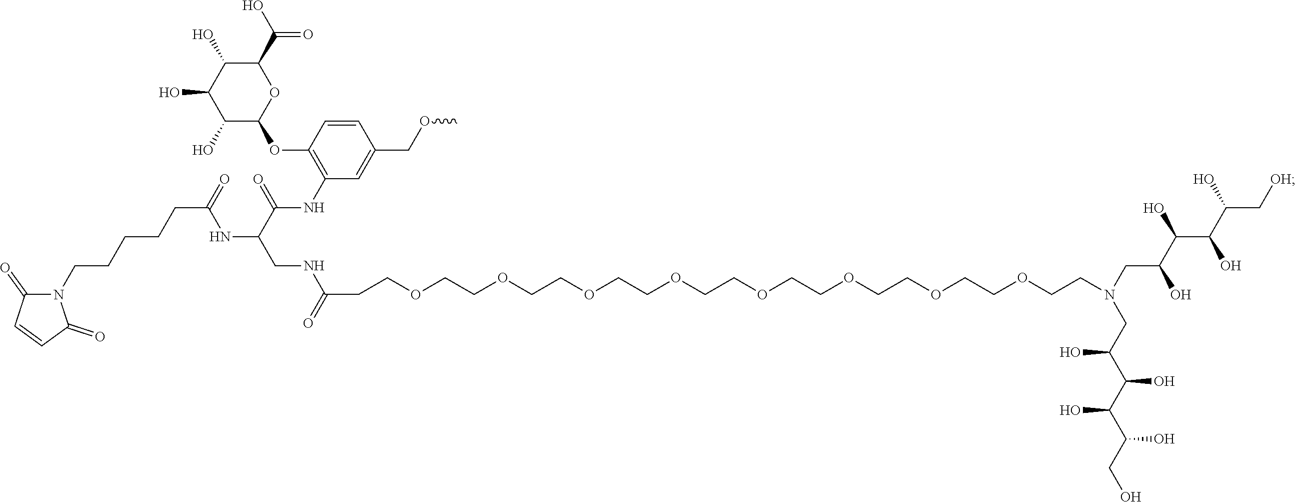

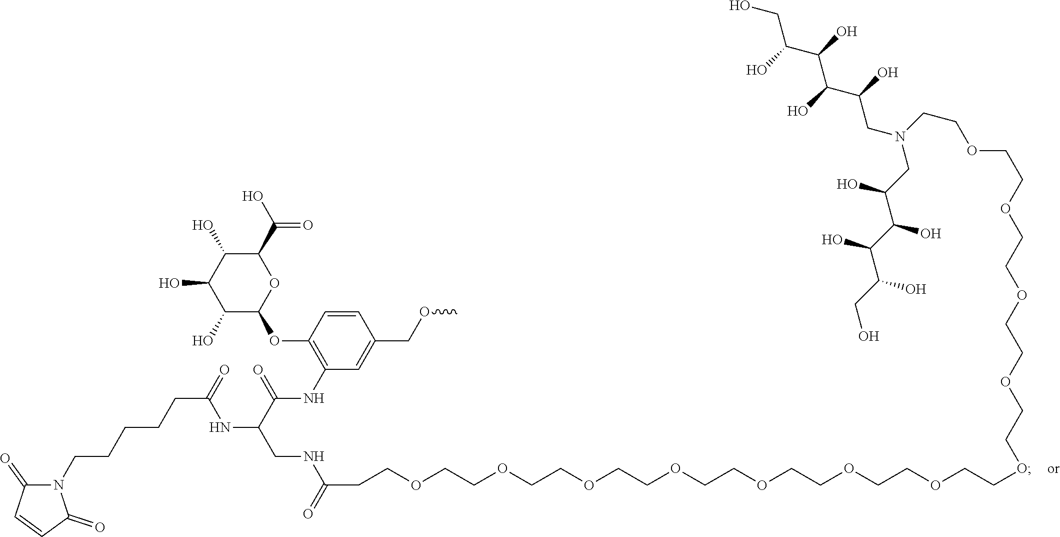

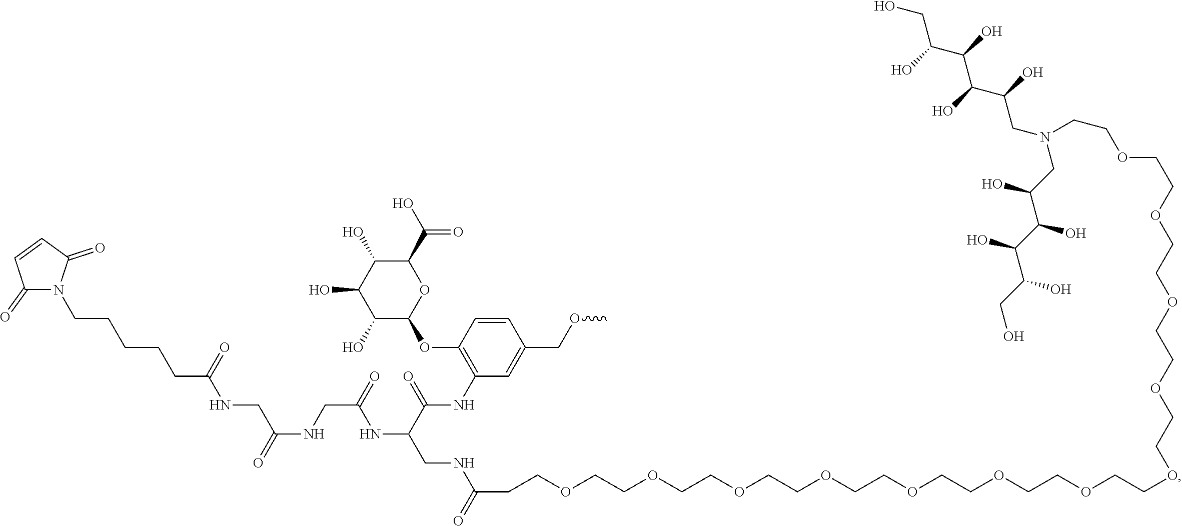

or a stereoisomer thereof, wherein the wavy line [symbol] indicates the attachment site to at least one of the Drug units or for a linking group attached to the at least one of the Drug units.

In some embodiments, provided is a Drug-Linker compound, comprising a Linker compound as described herein conjugated to at least one Drug unit.

In some embodiments, provided is a Drug-Linker, wherein the Drug unit is selected from a cytotoxic agent, an immune modulatory agent, a nucleic acid, a growth inhibitory agent, a PROTAC, a toxin, a radioactive isotope and a chelating ligand.

In some embodiments, provided is a Drug-Linker, wherein the Drug unit is a cytotoxic agent.

In some embodiments, provided is a Drug-Linker, wherein the cytotoxic agent is selected from the group consisting of an auristatin, a maytansinoid, a camptothecin, a duocarmycin, and a calicheamicin.

In some embodiments, provided is a Drug-Linker, wherein the cytotoxic agent is an auristatin.

In some embodiments, provided is a Drug-Linker, wherein the cytotoxic agent is MMAE or MMAF.

In some embodiments, provided is a Drug-Linker, wherein the cytotoxic agent is a camptothecin.

In some embodiments, provided is a Drug-Linker, wherein the cytotoxic agent is exatecan or SN-38.

In some embodiments, provided is a Drug-Linker, wherein the cytotoxic agent is RS-exatecan or SS-exatecan.

In some embodiments, provided is a Drug-Linker, wherein the cytotoxic agent is a calicheamicin.

In some embodiments, provided is a Drug-Linker, wherein the cytotoxic agent is a maytansinoid.

In some embodiments, provided is a Drug-Linker, wherein the maytansinoid is maytansine, maytansinol or ansamatocin-2.

In some embodiments, provided is a Drug-Linker, wherein the Drug unit is an immune modulatory agent.

In some embodiments, provided is a Drug-Linker, wherein the immune modulatory agent is selected from a TRL7 agonist, a TLR8 agonist, a STING agonist, or a RIG-I agonist.

In some embodiments, provided is a Drug-Linker, wherein the immune modulatory agent is an TLR7 agonist.

In some embodiments, provided is a Drug-Linker, wherein the TLR7 agonist is an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, or PolyG3.

In some embodiments, provided is a Drug-Linker, wherein the immune modulatory agent is a TLR8 agonist.

In some embodiments, provided is a Drug-Linker, wherein the TLR8 agonist is selected from an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA.

In some embodiments, provided is a Drug-Linker, wherein the immune modulatory agent is a STING agonist.

In some embodiments, provided is a Drug-Linker, wherein the immune modulatory agent is a RIG-I agonist.

In some embodiments, provided is a Drug-Linker, wherein the RIG-I agonist is selected from KIN1148, SB-9200, KIN700, KIN600, KIN500, KIN100, KIN101, KIN400 and KIN2000.

In some embodiments, provided is a Drug-Linker, wherein the Drug unit is a chelating ligand.

In some embodiments, provided is a Drug-Linker, wherein the chelating ligand is selected from platinum (Pt), ruthenium (Ru), rhodium (Rh), gold (Au), silver (Ag), copper (Cu), molybdenum (Mo), titanium (Ti), or iridum (Ir); a radioisotope such as yttrium-88, yttrium-90, technetium-99, copper-67, rhenium-188, rhenium-186, gallium-66, gallium-67, indium-111, indium-114, indium-115, lutetium-177, strontium-89, sacrarium-153, and lead-212.

In some embodiments, provided is a Drug-Linker, having one of the following structures:

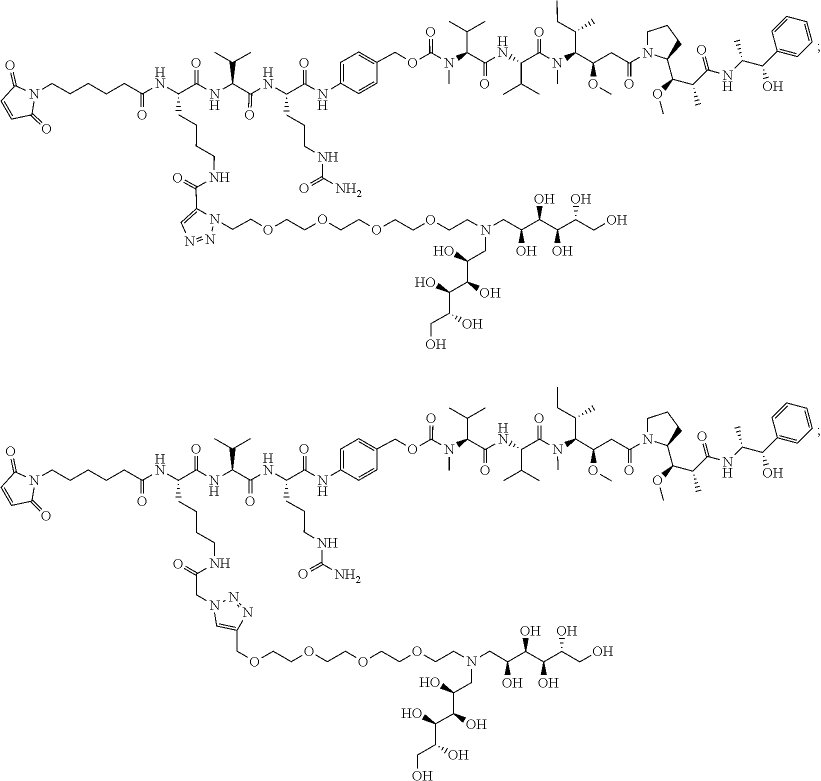

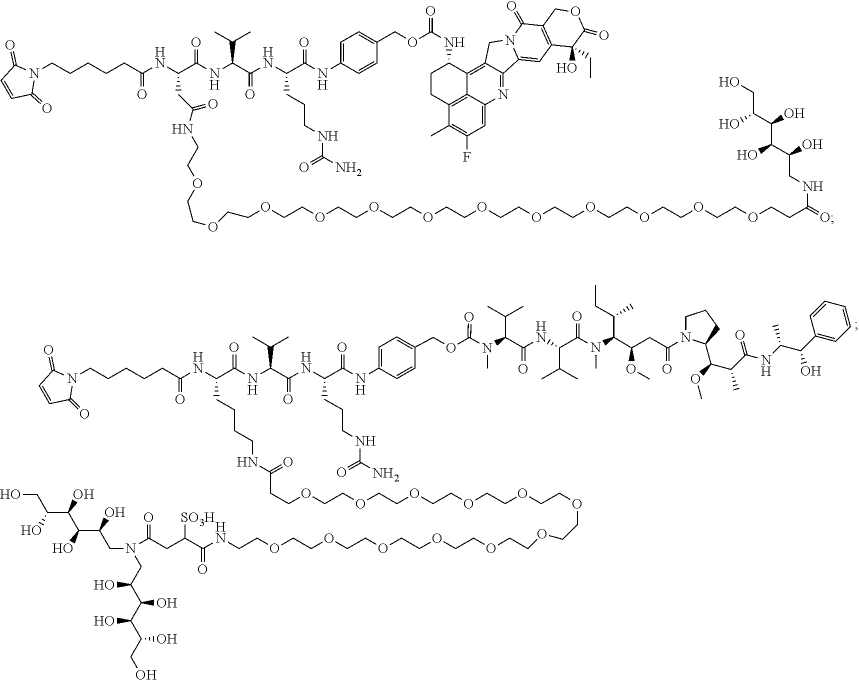

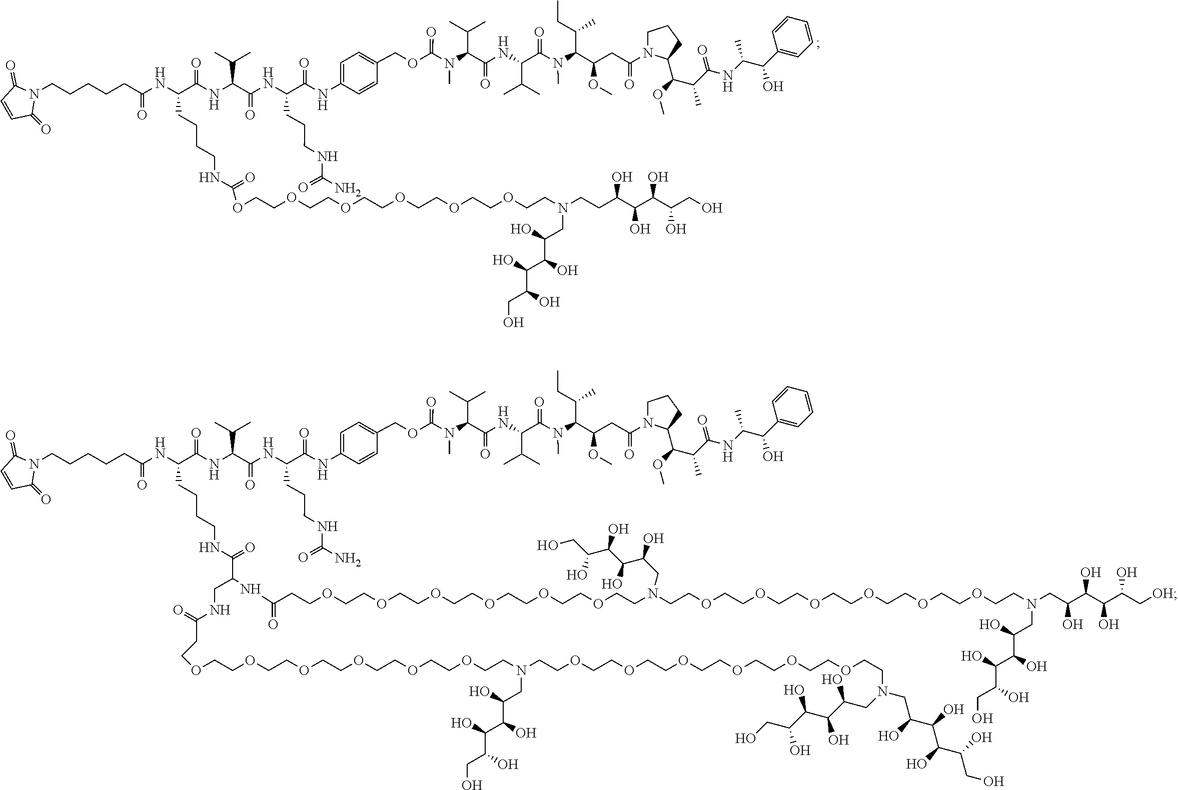

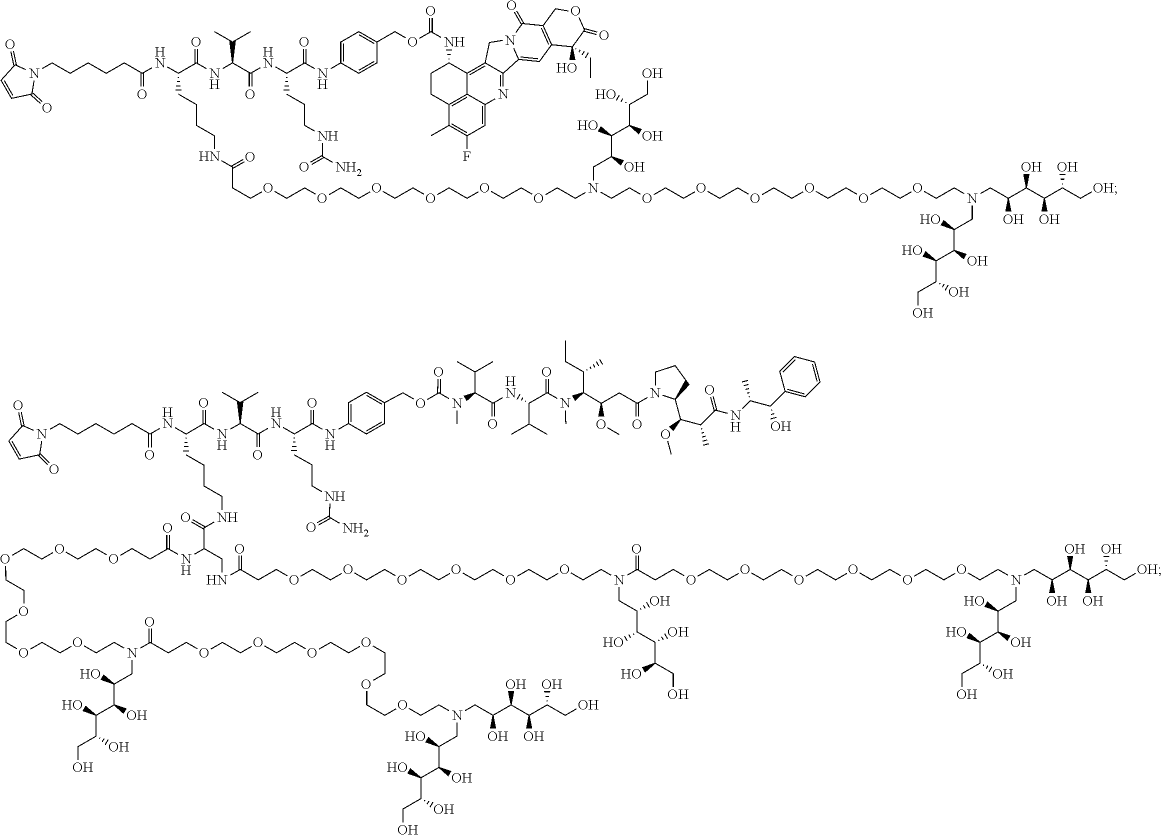

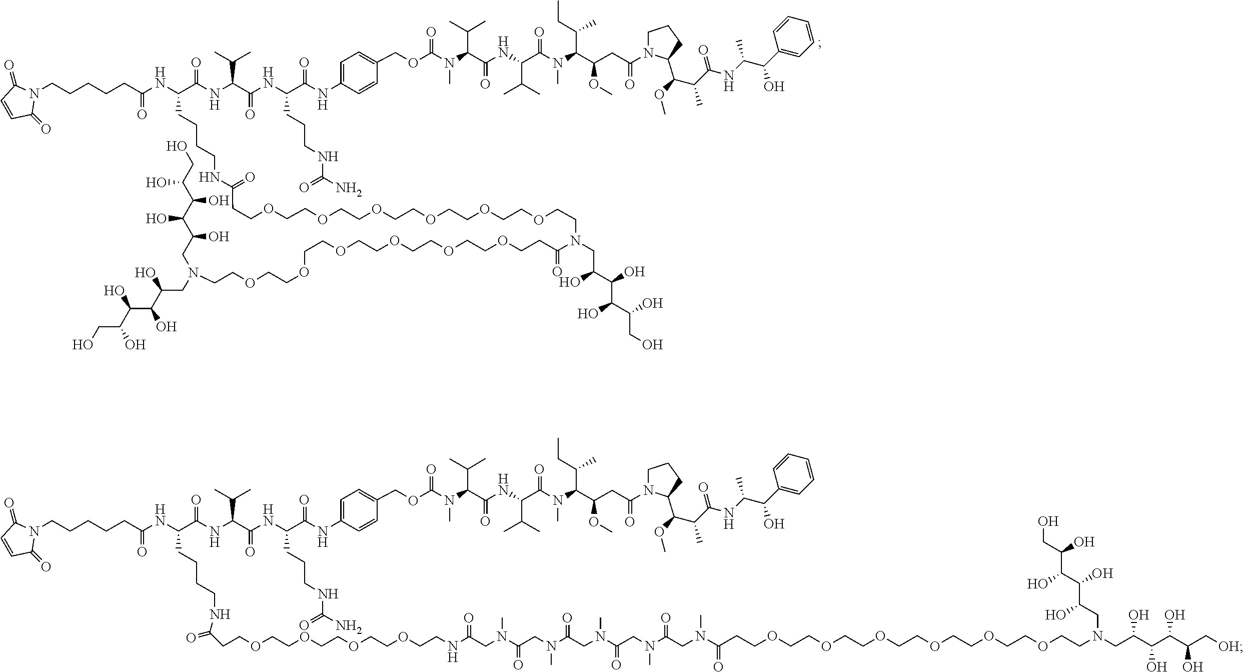

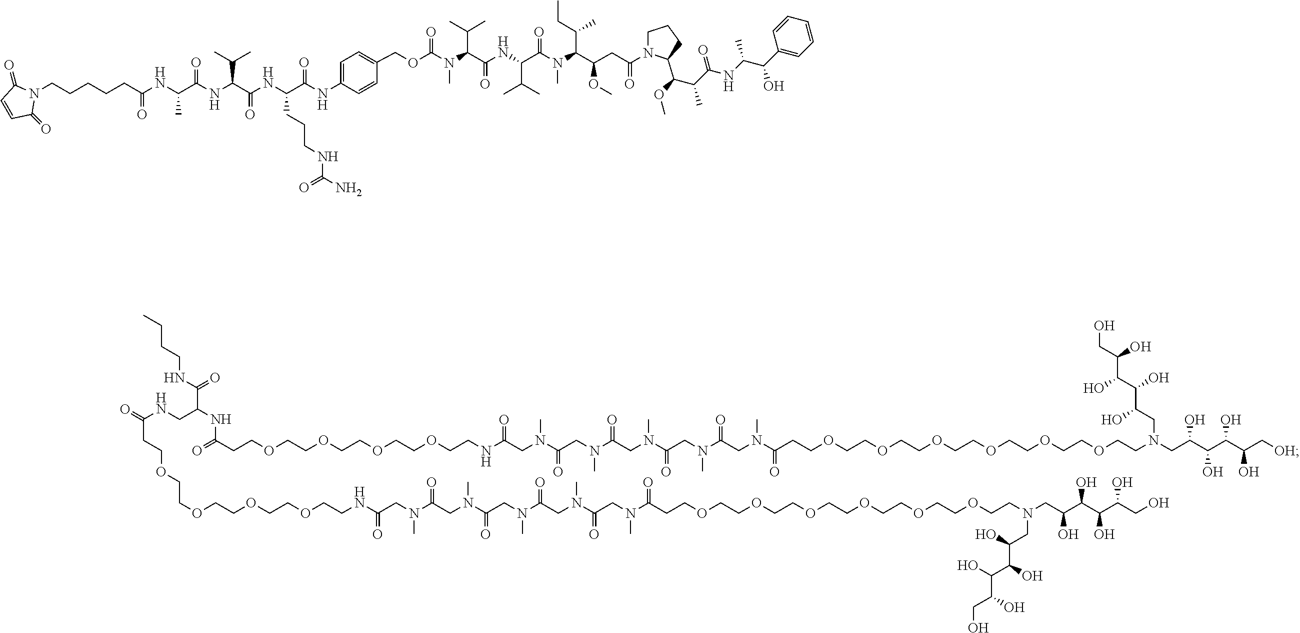

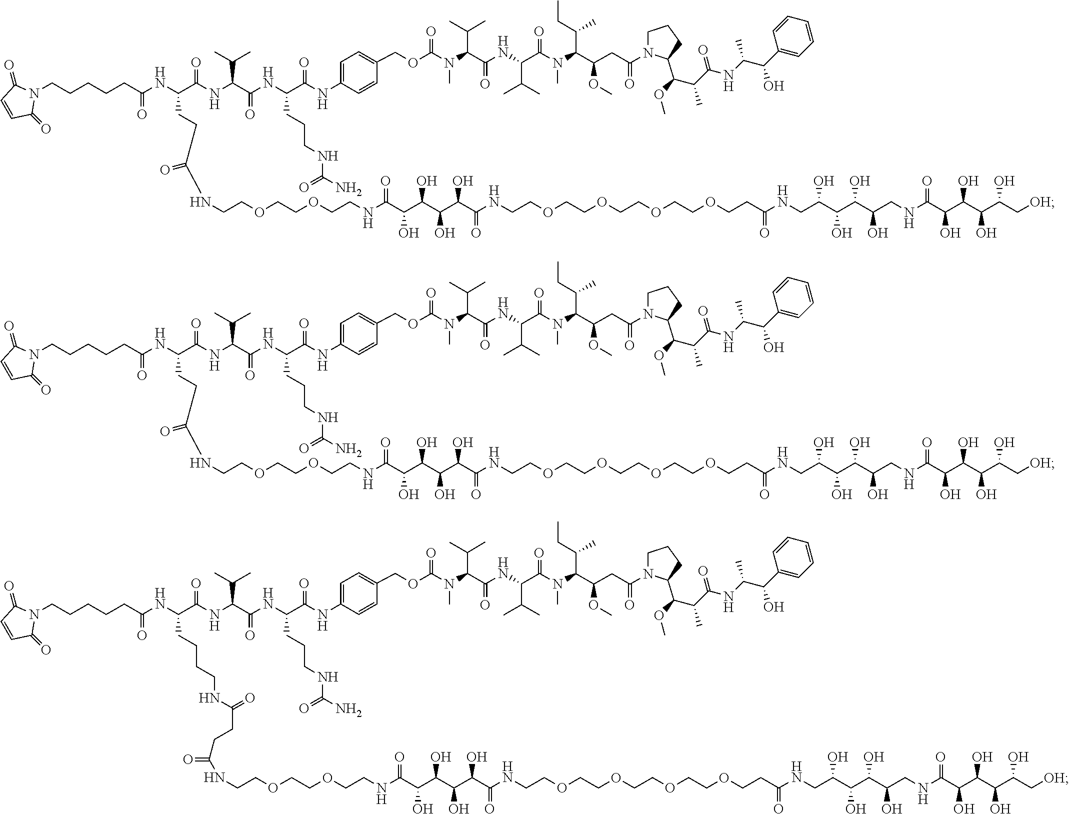

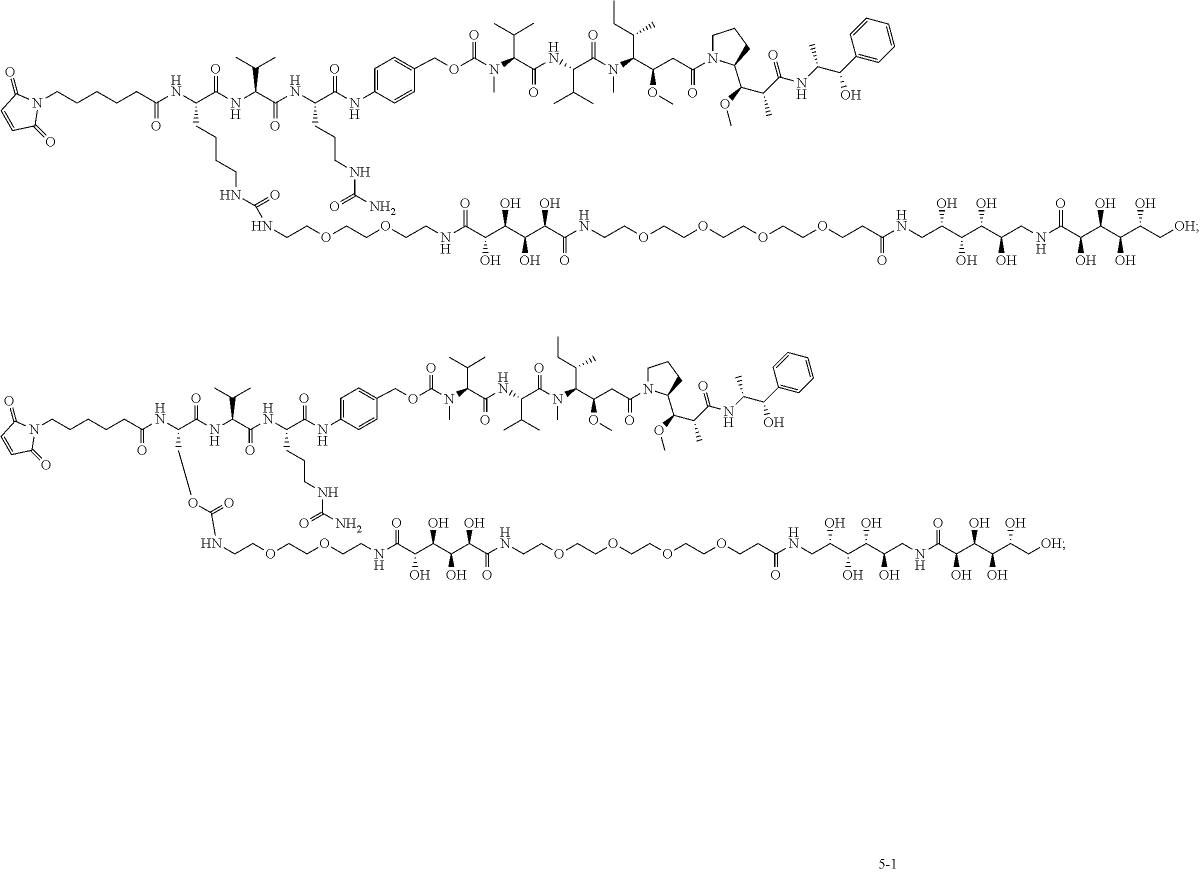

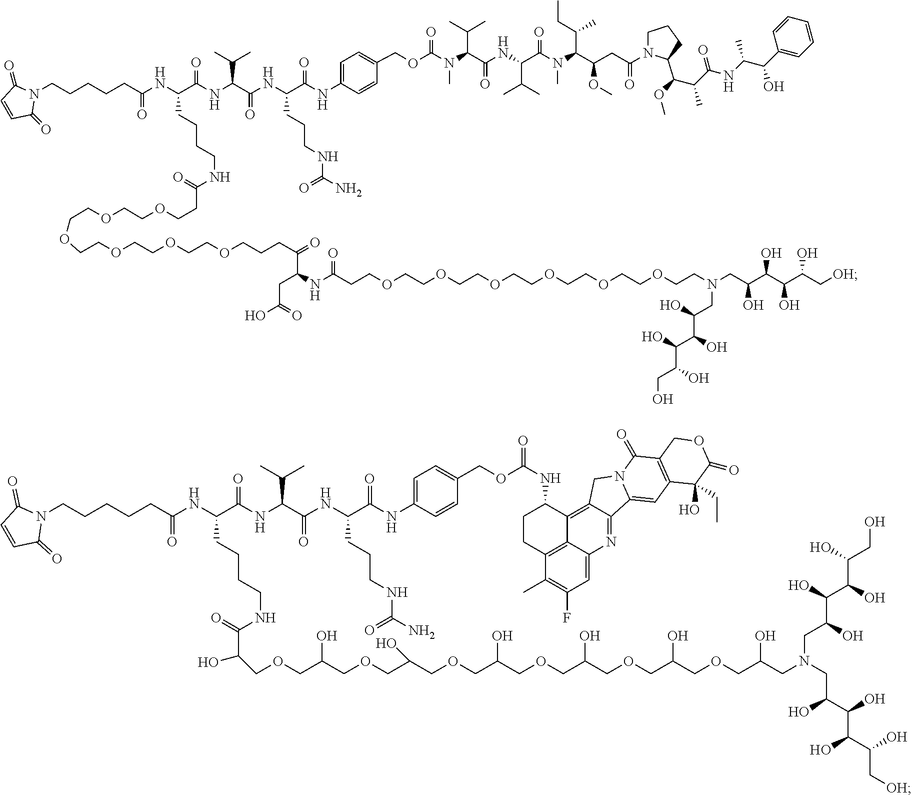

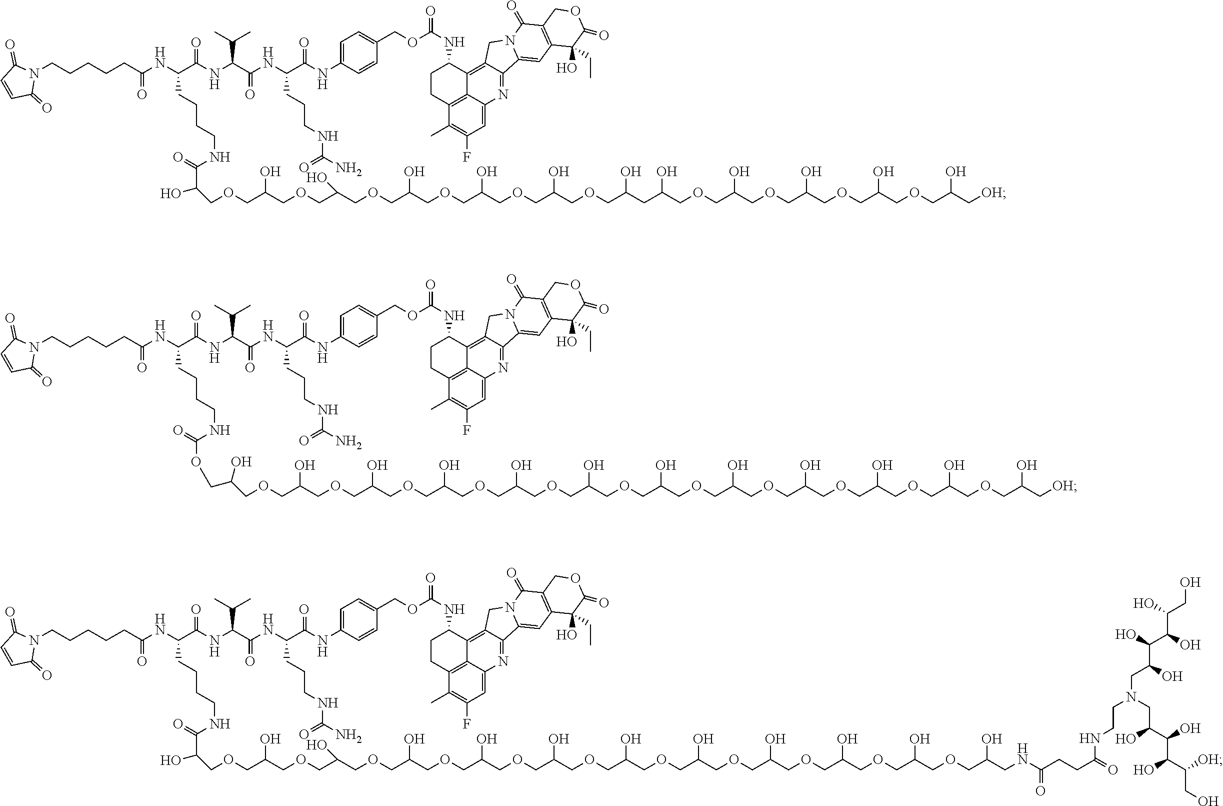

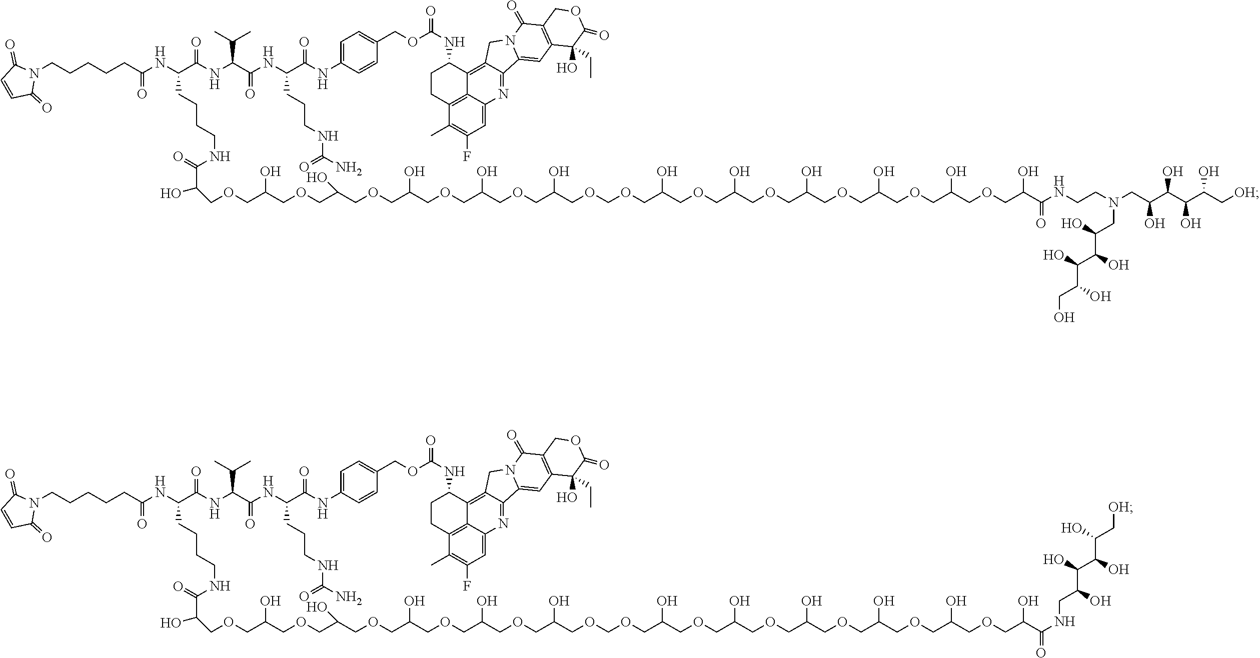

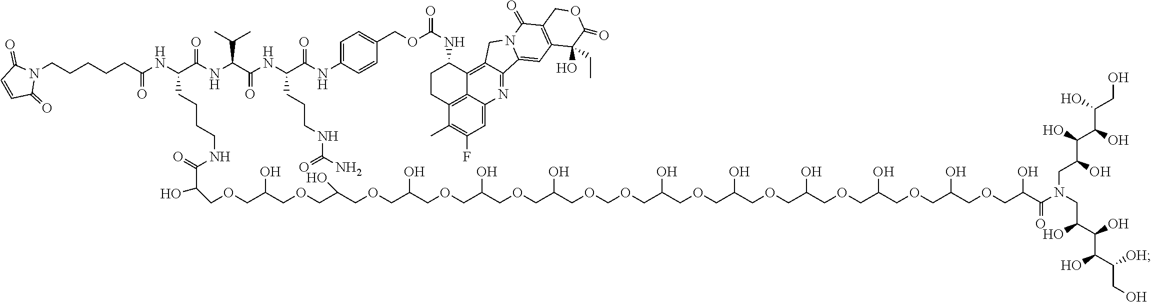 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)[C@H](CCCCNC(=O)C(O)COCC(O)COCC(O)COCC(O)COCC(O)COCC(O)COCOCC(O)COCC(O)COCC(O)COCC(O)COCC(O)COCC(O)C(=O)N(C[C@H](O)[C@@H](O)[C@H](O)[C@H](O)CO)C[C@H](O)[C@@H](O)[C@H](O)[C@H](O)CO)NC(=O)CCCCCN2C(=O)C=CC2=O)C(C)C)cc1)CC3]

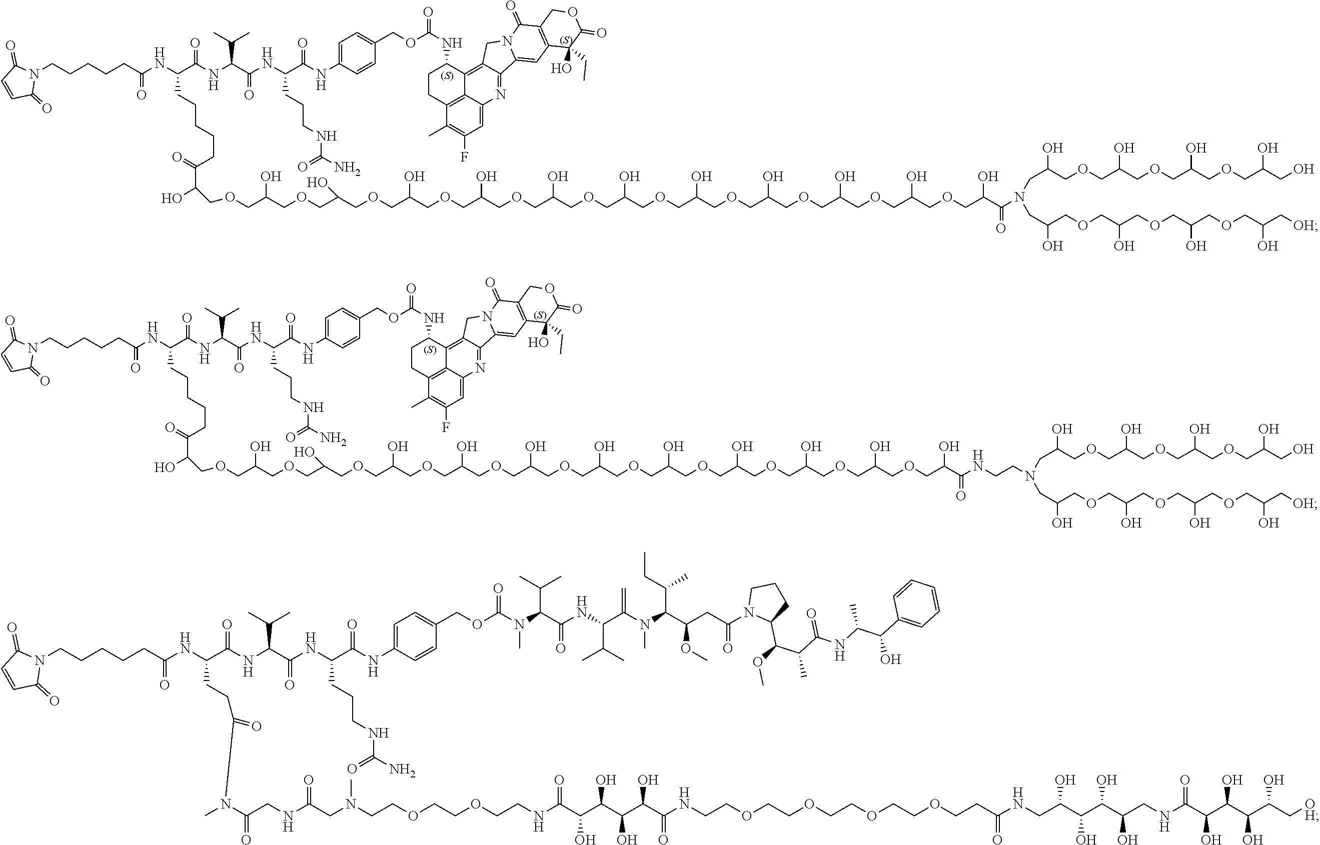

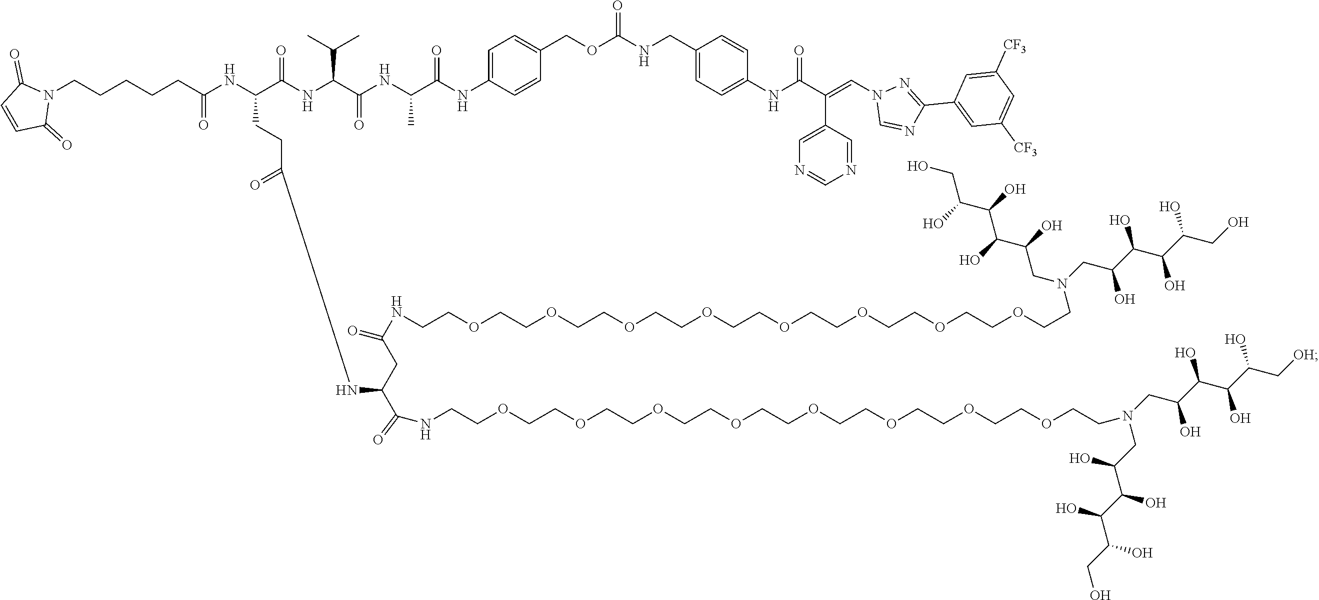 [SMILES: CC(C)[C@H](NC(=O)[C@H](CCC(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN1C(=O)C=CC1=O)C(=O)N[C@@H](C)C(=O)Nc1ccc(COC(=O)NCc2ccc(NC(=O)/C(=C/n3cnc(-c4cc(C(F)(F)F)cc(C(F)(F)F)c4)n3)c3cncnc3)cc2)cc1]

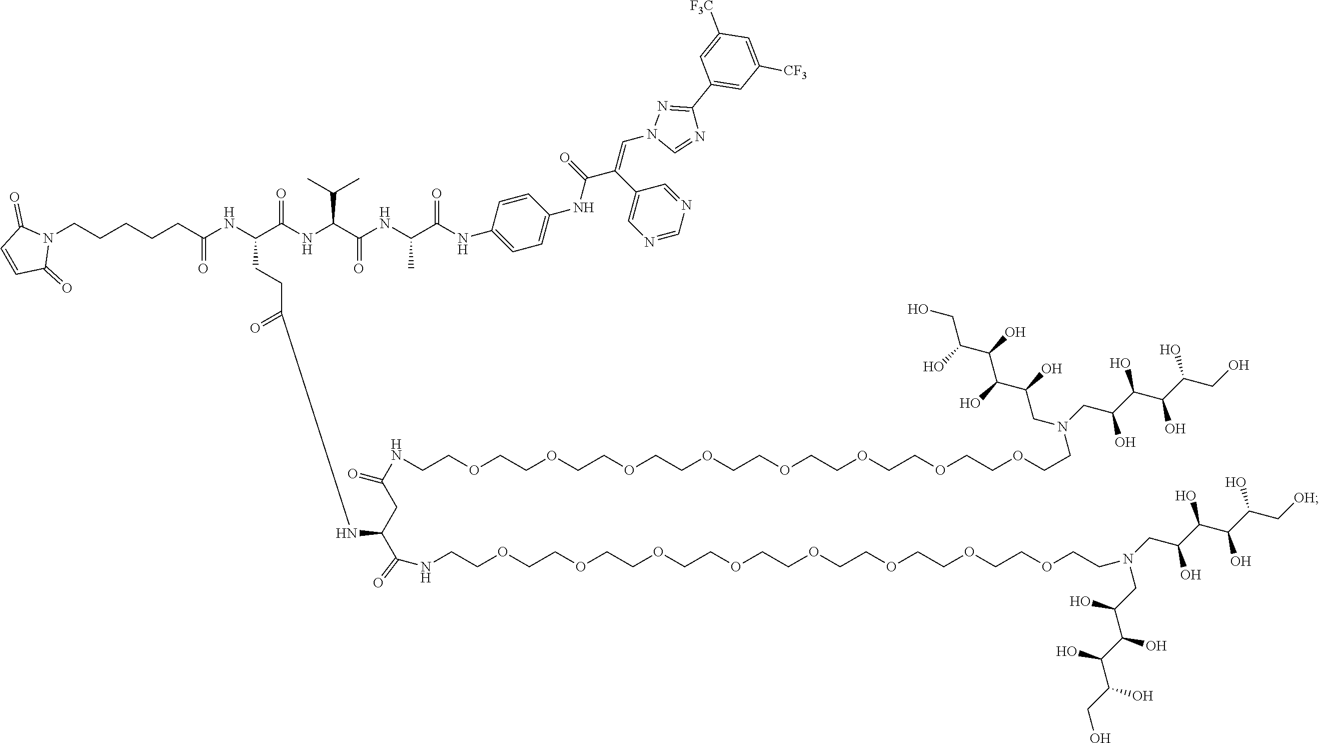 [SMILES: CC(C)[C@H](NC(=O)[C@H](CCC(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN1C(=O)C=CC1=O)C(=O)N[C@@H](C)C(=O)Nc1ccc(NC(=O)/C(=C/n2cnc(-c3cc(C(F)(F)F)cc(C(F)(F)F)c3)n2)c2cncnc2)cc1]

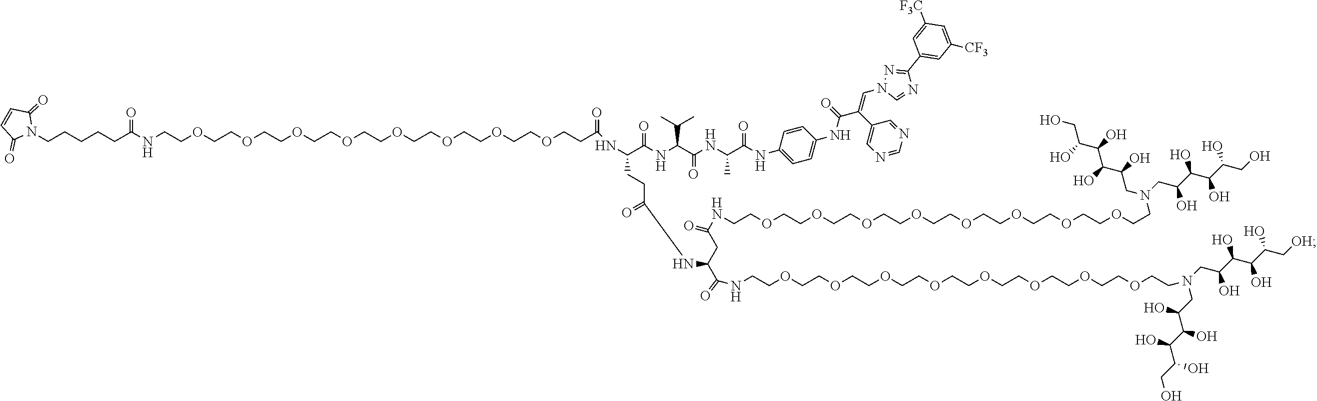 [SMILES: CC(C)[C@H](NC(=O)[C@H](CCC(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCOCCOCCOCCOCCOCCOCCOCCOCCNC(=O)CCCCCN1C(=O)C=CC1=O)C(=O)N[C@@H](C)C(=O)Nc1ccc(NC(=O)/C(=C/n2cnc(-c3cc(C(F)(F)F)cc(C(F)(F)F)c3)n2)c2cncnc2)cc1]

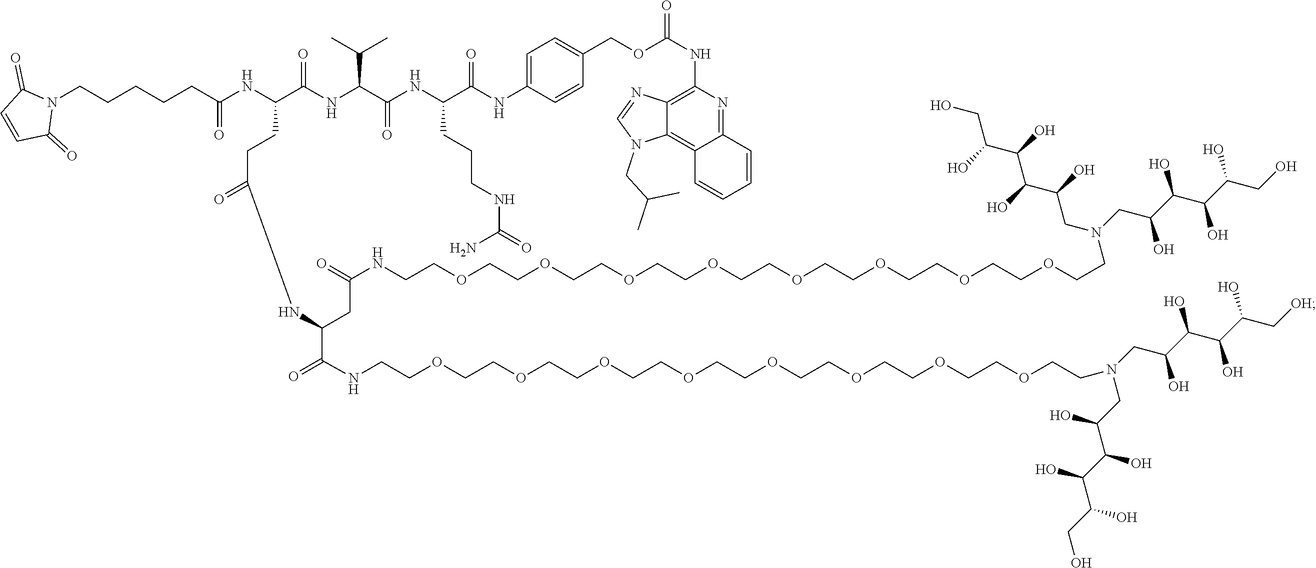 [SMILES: CC(C)Cn1cnc2c(NC(=O)OCc3ccc(NC(=O)[C@H](CCCNC(N)=O)NC(=O)[C@@H](NC(=O)[C@H](CCC(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN4C(=O)C=CC4=O)C(C)C)cc3)nc3ccccc3c21]

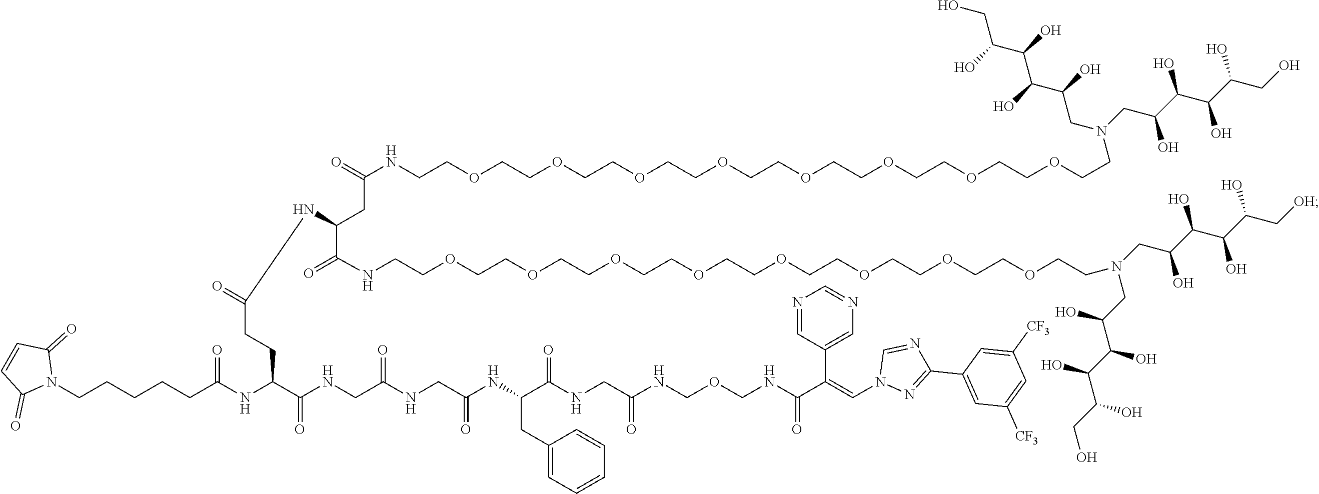 [SMILES: O=C(CNC(=O)[C@H](CCC(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN1C(=O)C=CC1=O)NCC(=O)N[C@@H](Cc1ccccc1)C(=O)NCC(=O)NCOCNC(=O)/C(=C/n1cnc(-c2cc(C(F)(F)F)cc(C(F)(F)F)c2)n1)c1cncnc1]

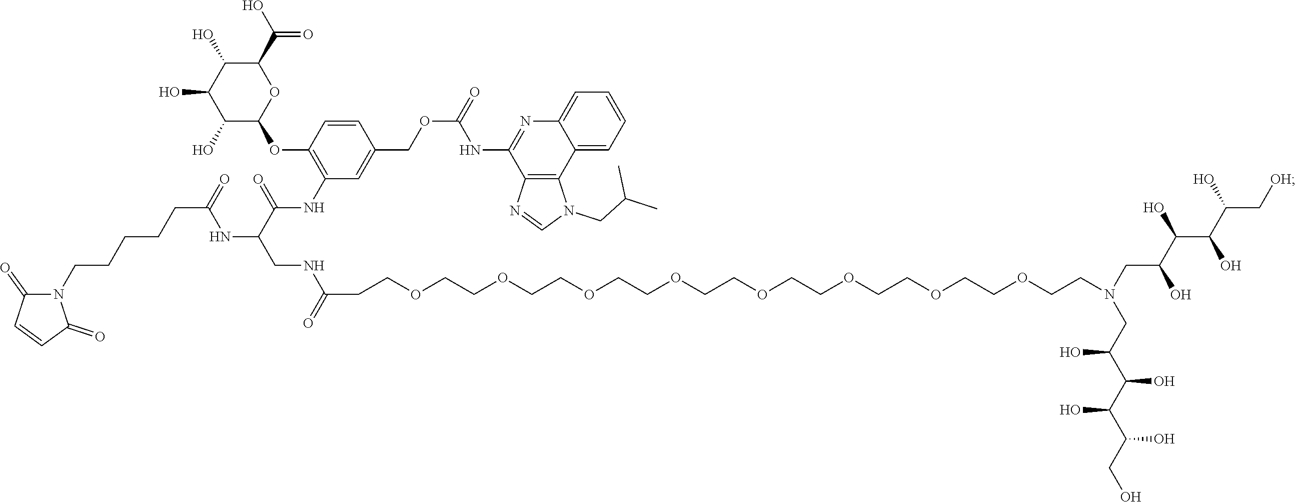 [SMILES: CC(C)Cn1cnc2c(NC(=O)OCc3ccc(O[C@@H]4O[C@H](C(=O)O)[C@@H](O)[C@H](O)[C@H]4O)c(NC(=O)C(CNC(=O)CCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN4C(=O)C=CC4=O)c3)nc3ccccc3c21]

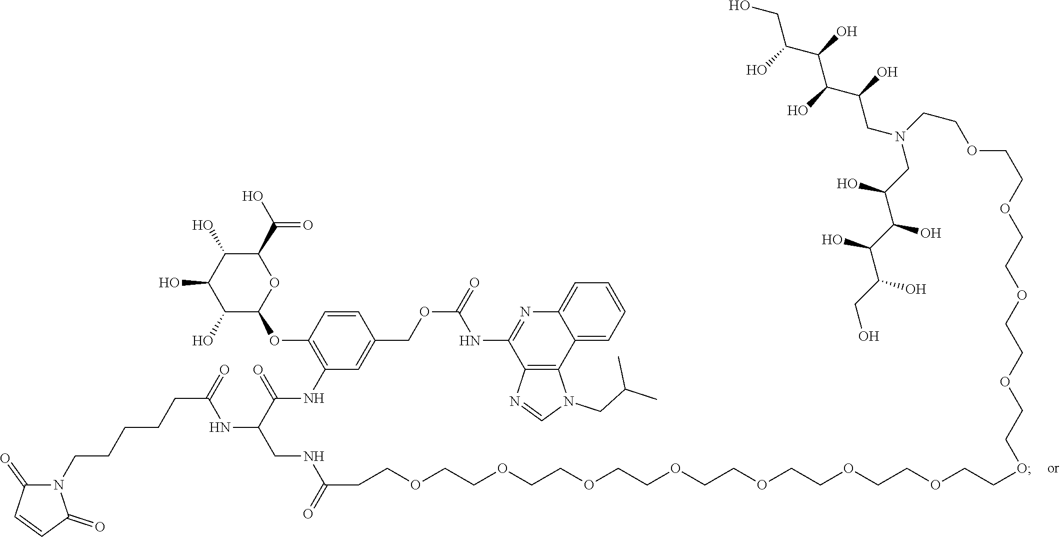 [SMILES: CC(C)Cn1cnc2c(NC(=O)OCc3ccc(O[C@@H]4O[C@H](C(=O)O)[C@@H](O)[C@H](O)[C@H]4O)c(NC(=O)C(CNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN4C(=O)C=CC4=O)c3)nc3ccccc3c21 ; "or"]

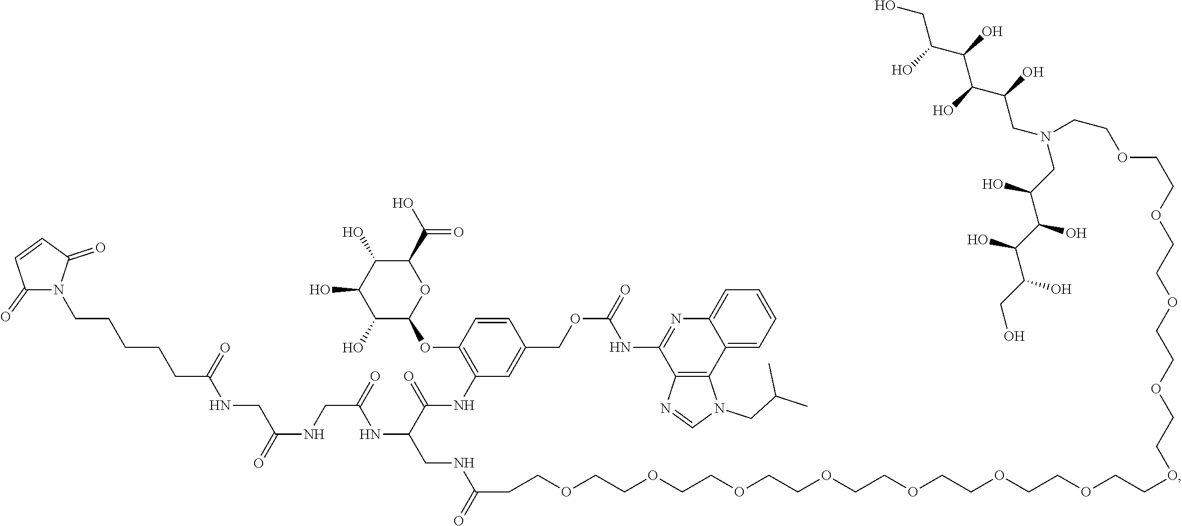 [SMILES: CC(C)Cn1cnc2c(NC(=O)OCc3ccc(O[C@@H]4O[C@H](C(=O)O)[C@@H](O)[C@H](O)[C@H]4O)c(NC(=O)C(CNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CNC(=O)CNC(=O)CCCCCN4C(=O)C=CC4=O)c3)nc3ccccc3c21]

or a stereoisomer thereof.

In some embodiments, provided is a conjugate comprising a Targeting unit attached to a Drug-linker as described herein, wherein the Targeting unit specifically binds to a target molecule.

In some embodiments, provided is a conjugate, wherein the Targeting unit is selected from an antibody or an antigen-binding portion thereof.

In some embodiments, provided is a conjugate, wherein the Targeting unit is a monoclonal antibody, a Fab, a Fab′, an F(ab′), an Fv, a disulfide linked Fc, a scFv, a single domain antibody, a diabody, a bi-specific antibody, or a multi-specific antibody.

In some embodiments, provided is a conjugate, wherein the Targeting unit is selected from: a scFv1-ScFv2, a ScFv12-Fc-scFv22, a IgG-scFv, a DVD-Ig, a triomab/quadroma, a two-in-one IgG, a scFv2-Fc, a TandAb, and an scFv-HSA-scFv.

In some embodiments, provided is a conjugate, wherein the Targeting unit is a diabody, a DART, an anticalin, an affibody, an avimer, a DARPin, or an adnectin.

In some embodiments, provided is a conjugate, wherein the Targeting unit is mono-specific.

In some embodiments, provided is a conjugate, wherein the Targeting unit is bivalent.

In some embodiments, provided is a conjugate, wherein the Targeting unit is bispecific.

In some embodiments, provided is a conjugate, wherein the average drug loading (p^{load}) of the conjugate is from about 1 to about 8, about 2, about 4, about 6, about 8, about 10, about 12, about 14, about 16, about 3 to about 5, about 6 to about 8, or about 8 to about 16.

In some embodiments, provided is a conjugate, selected from the following:

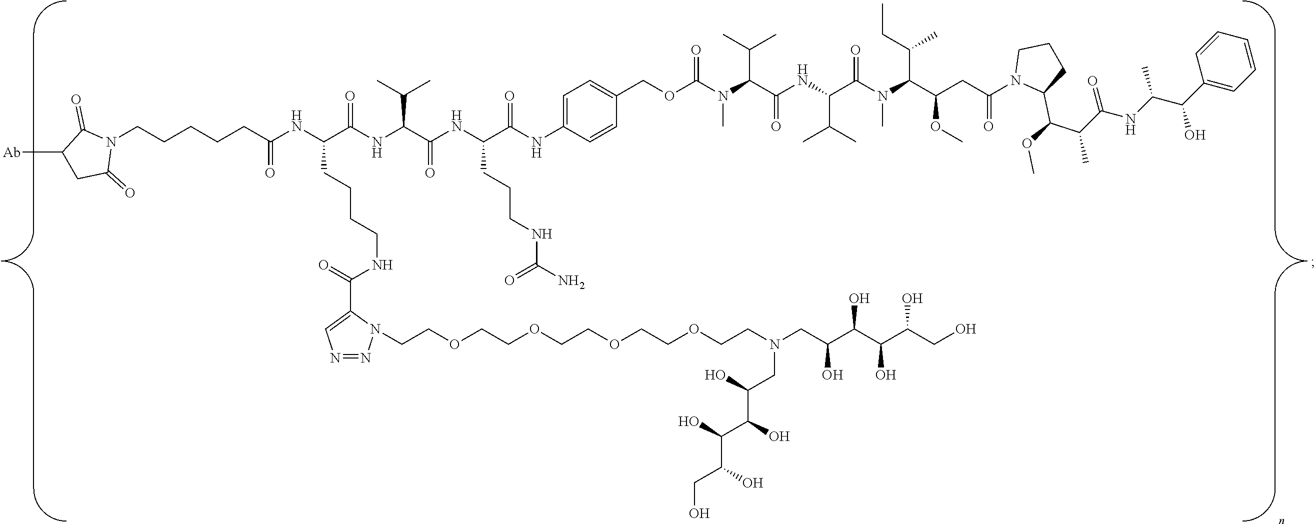

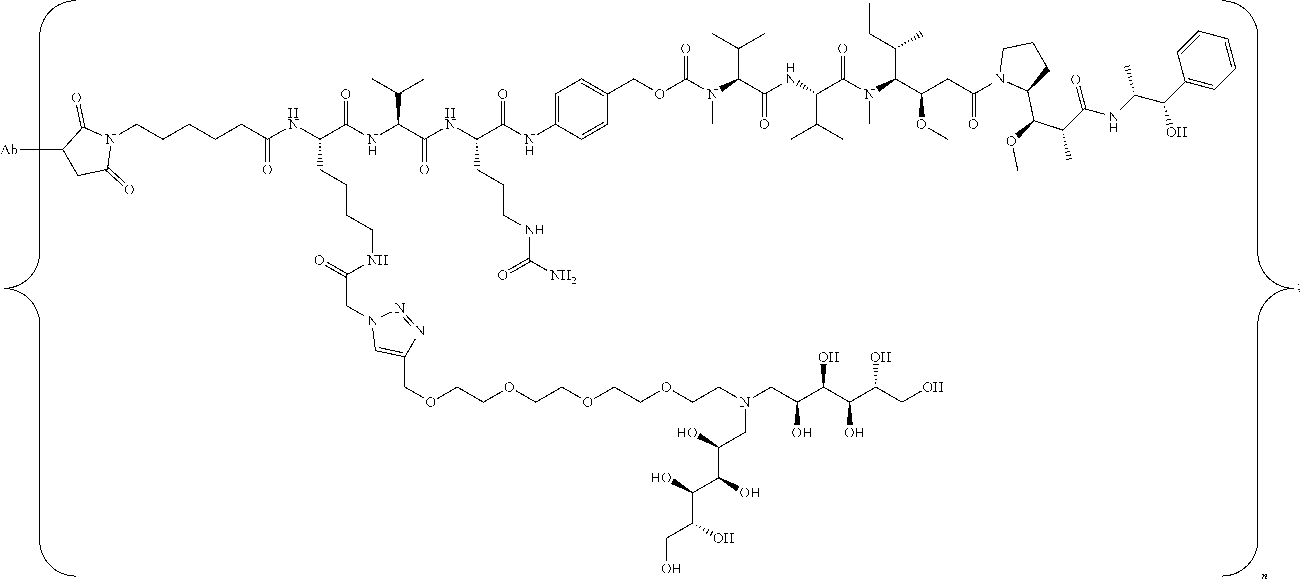

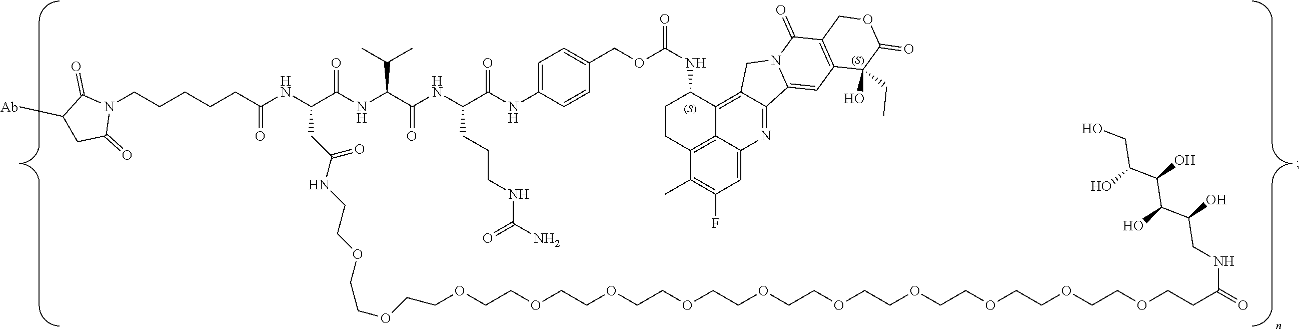

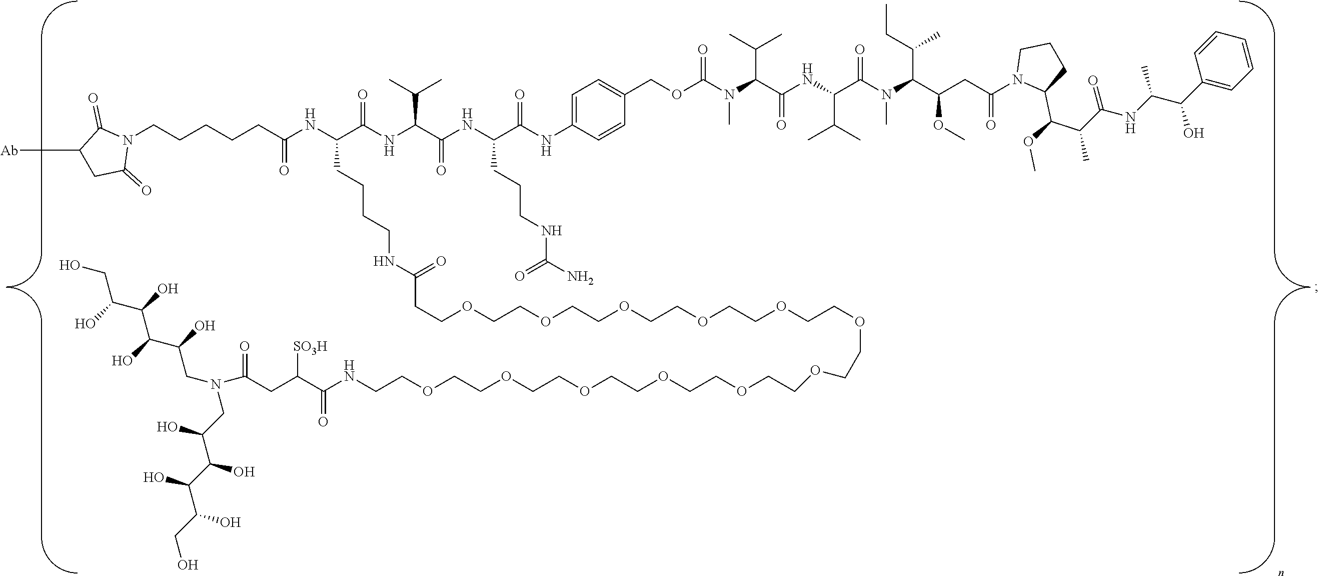

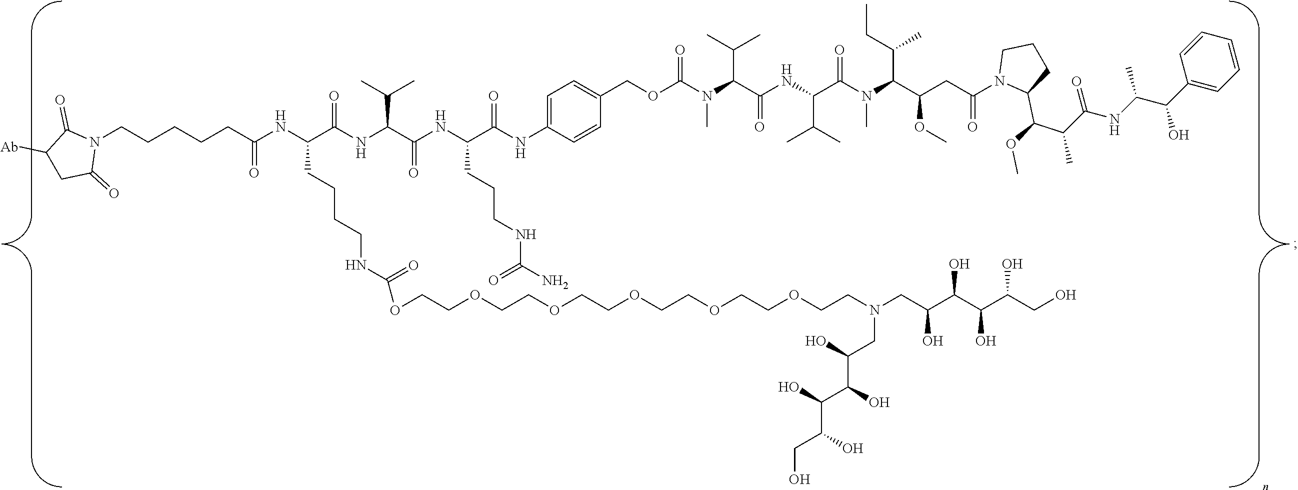

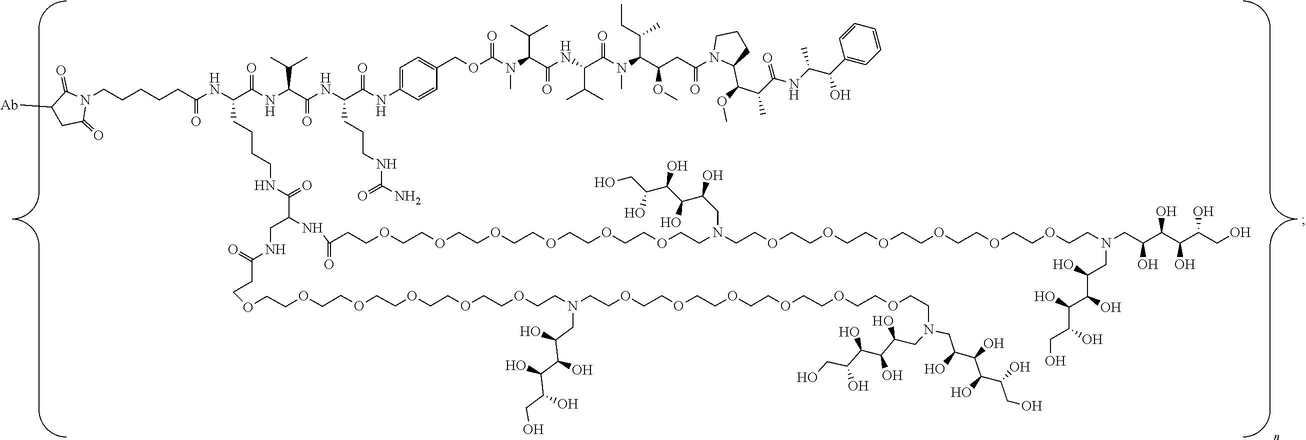

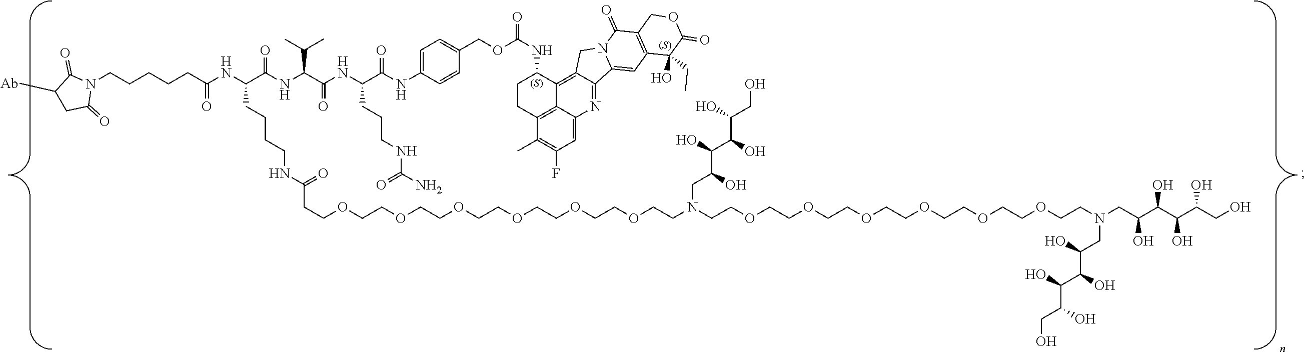

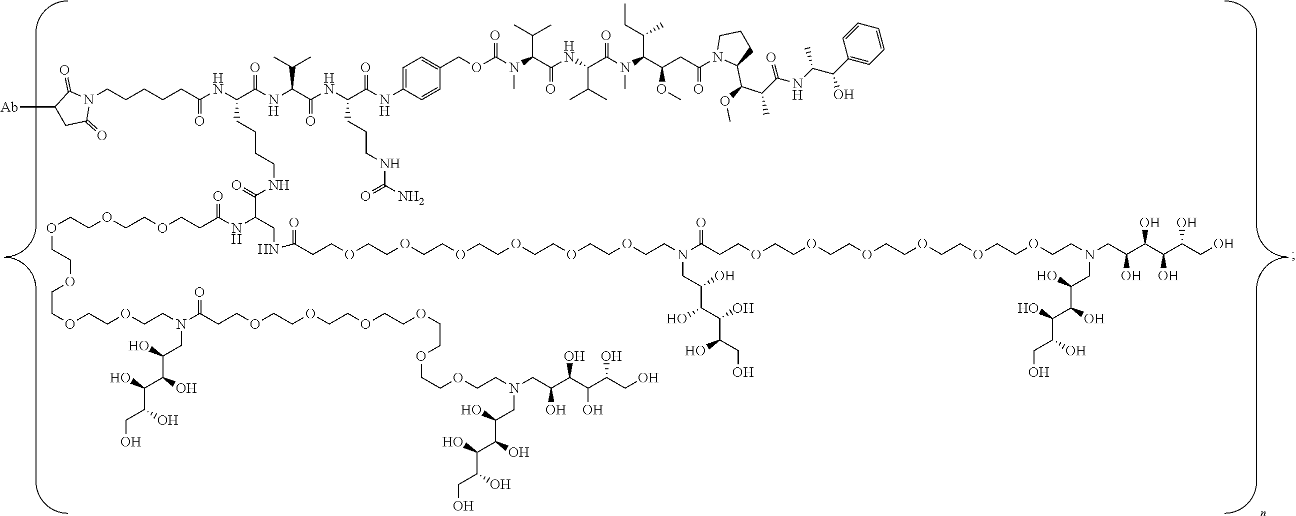

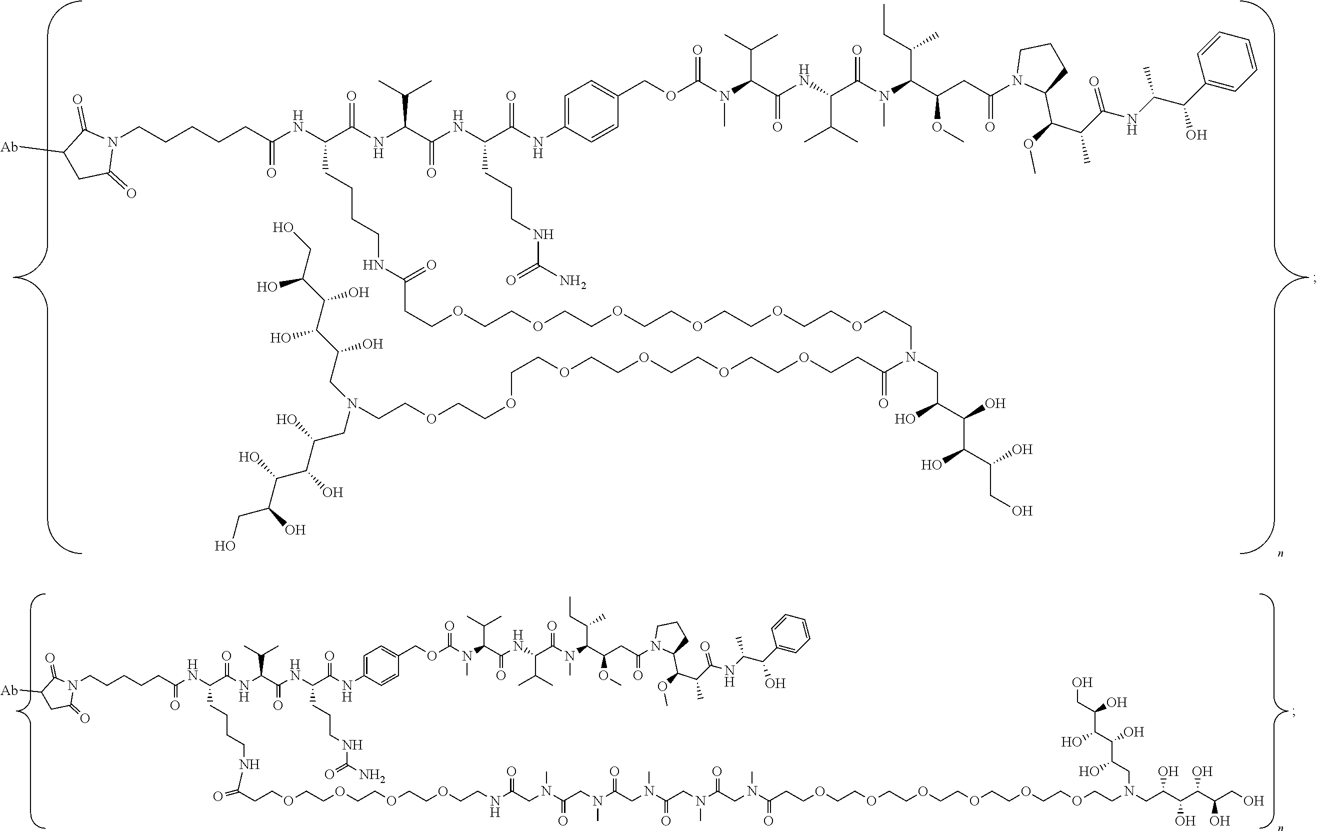

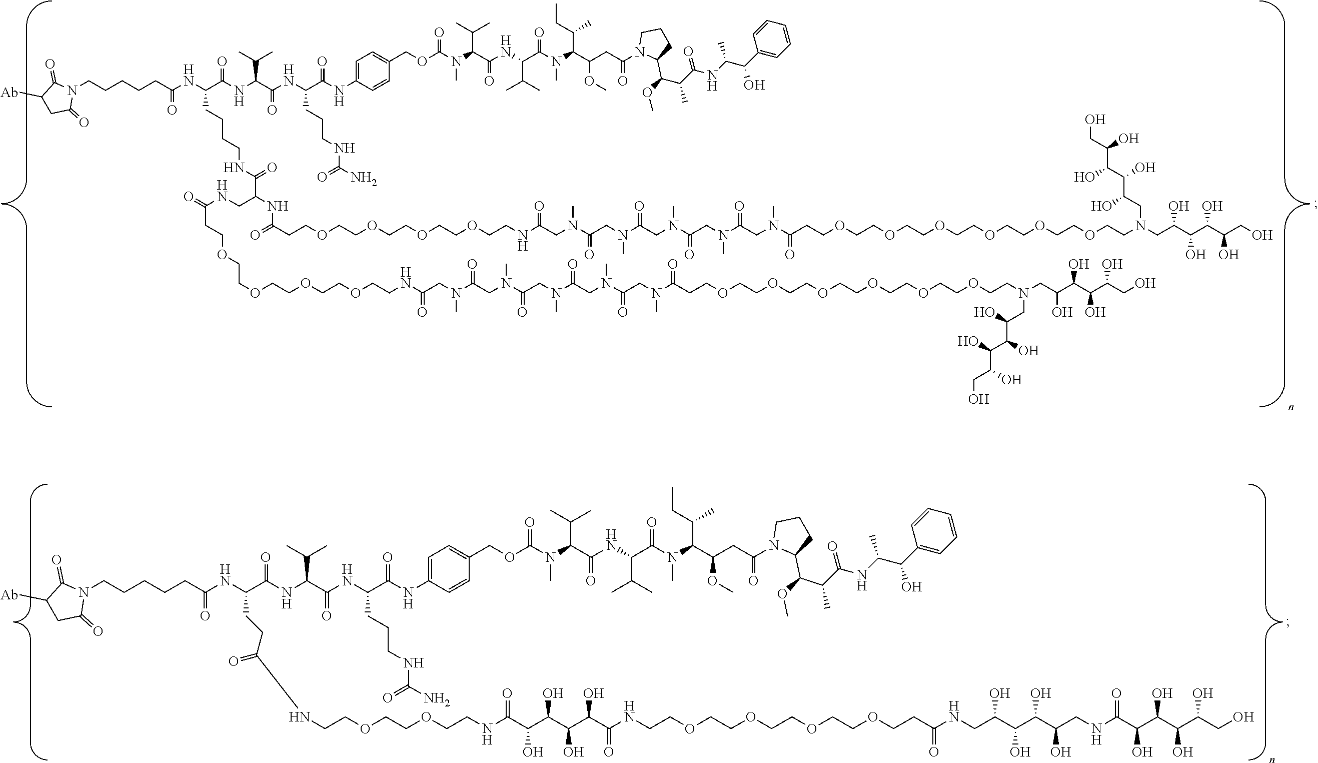

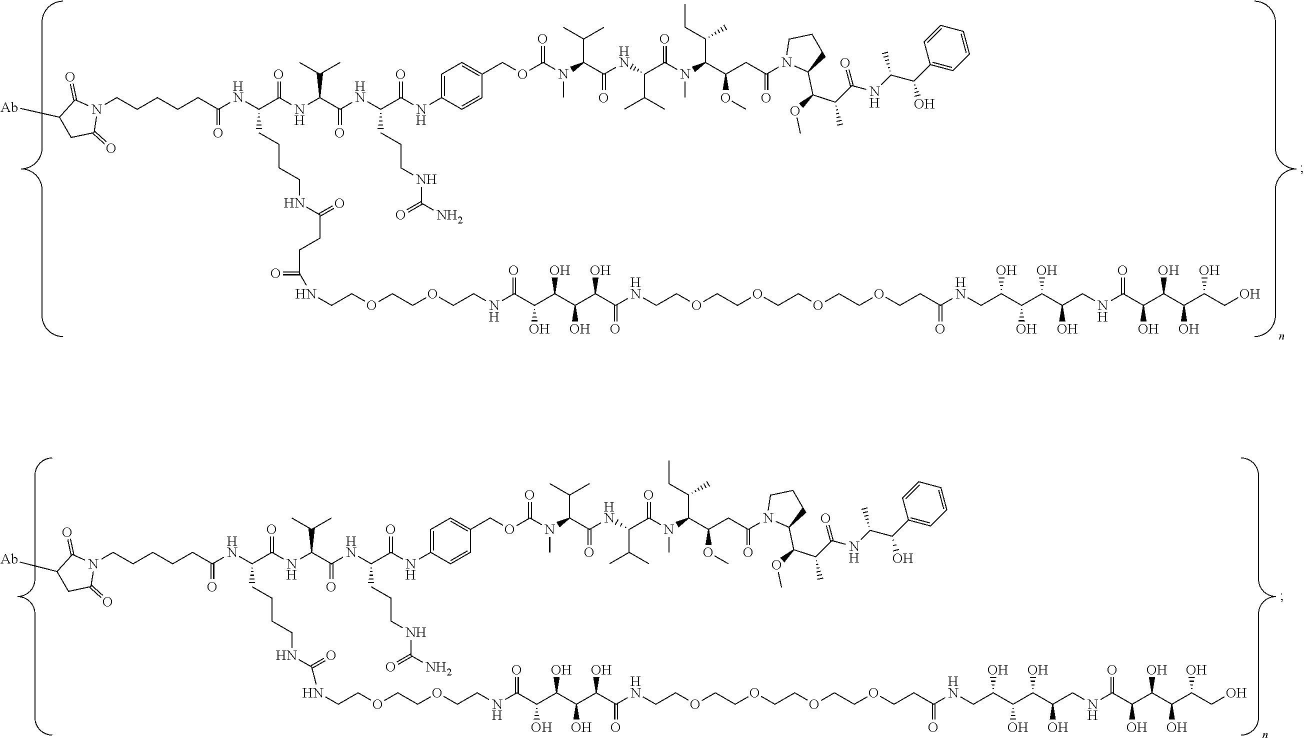

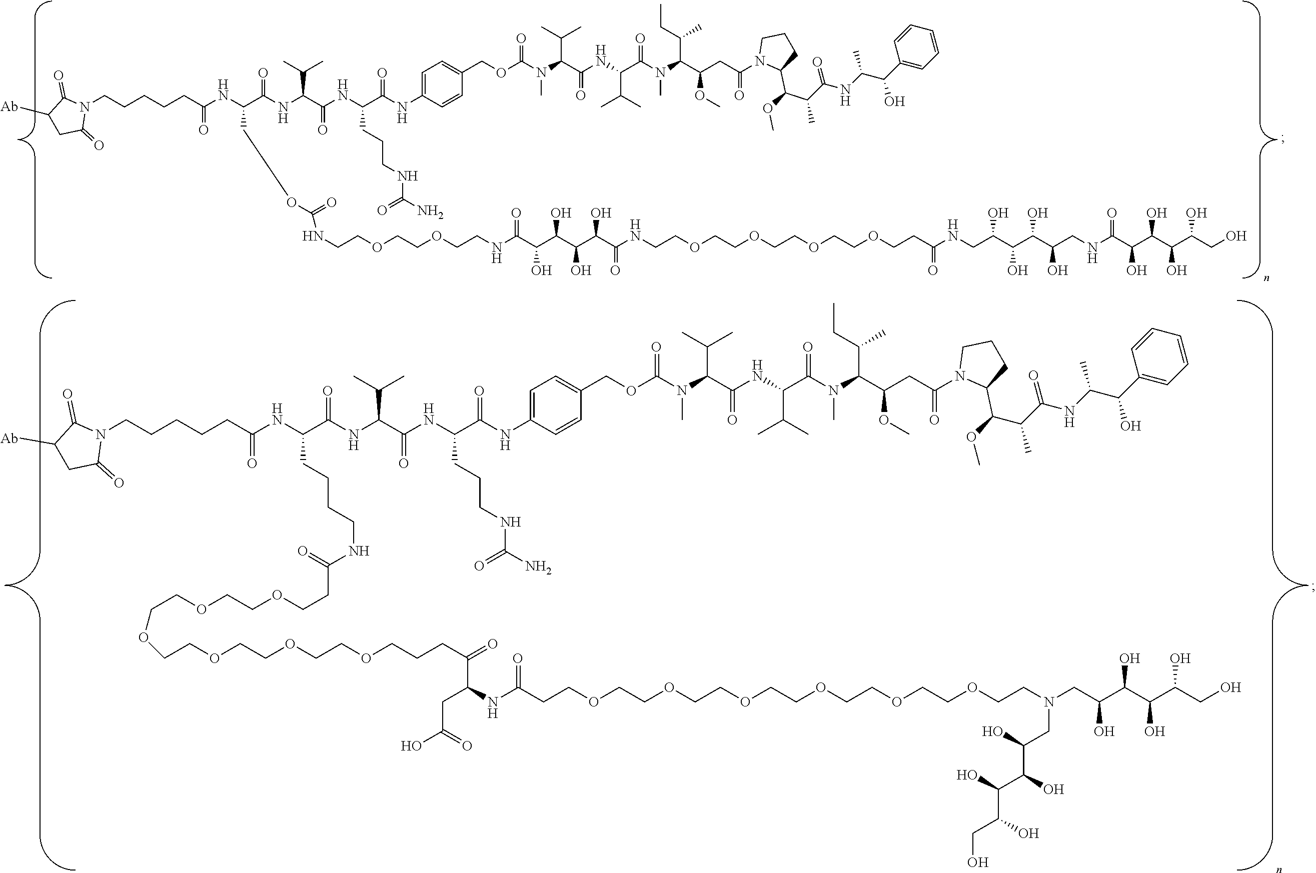

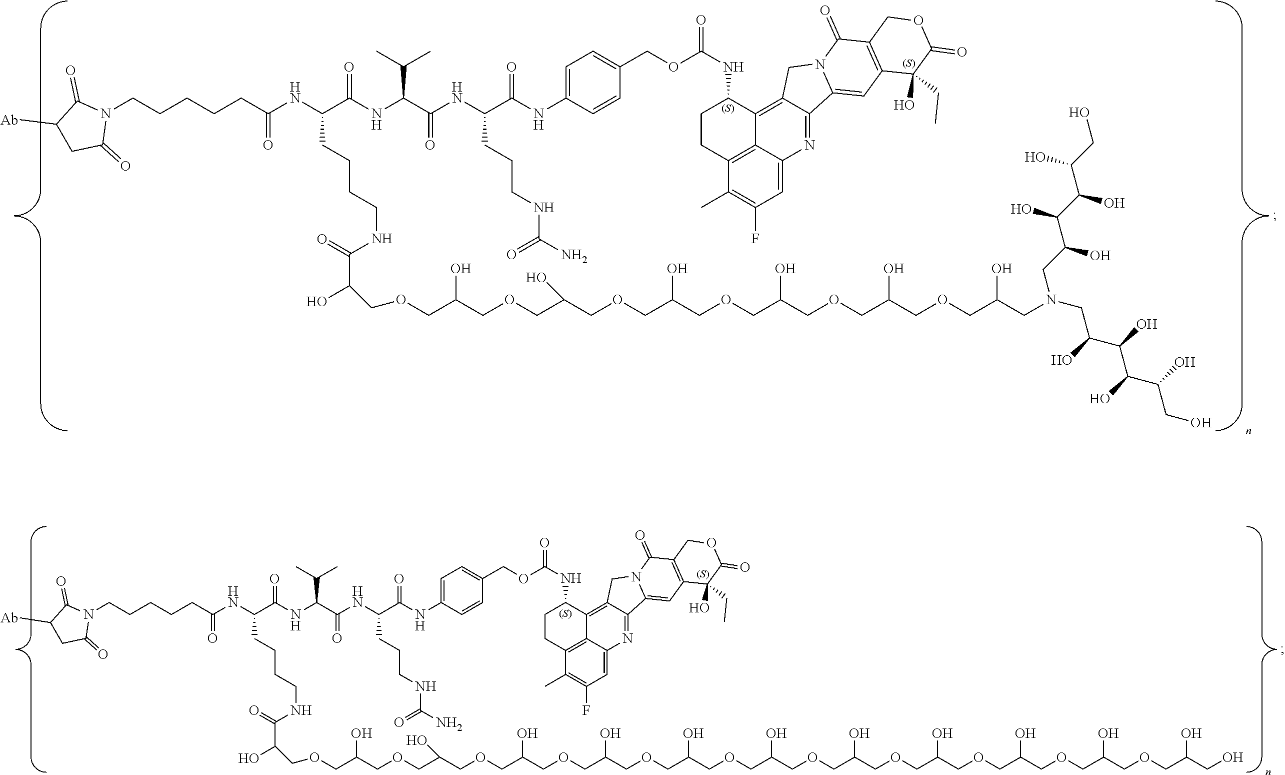

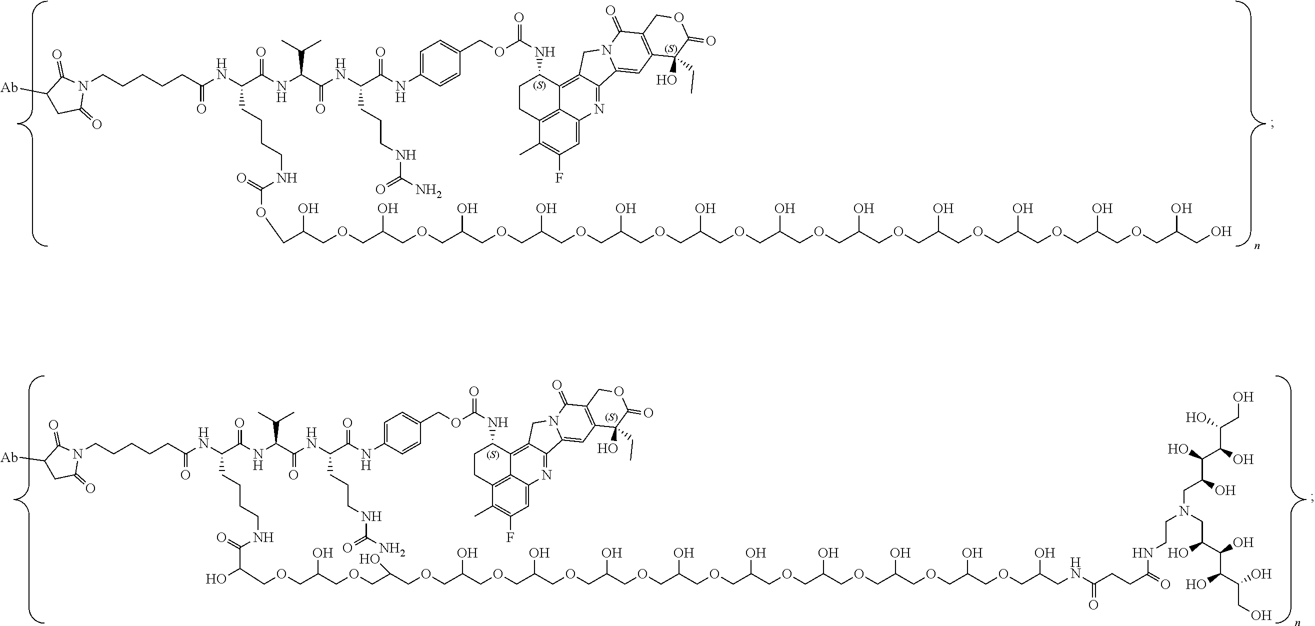

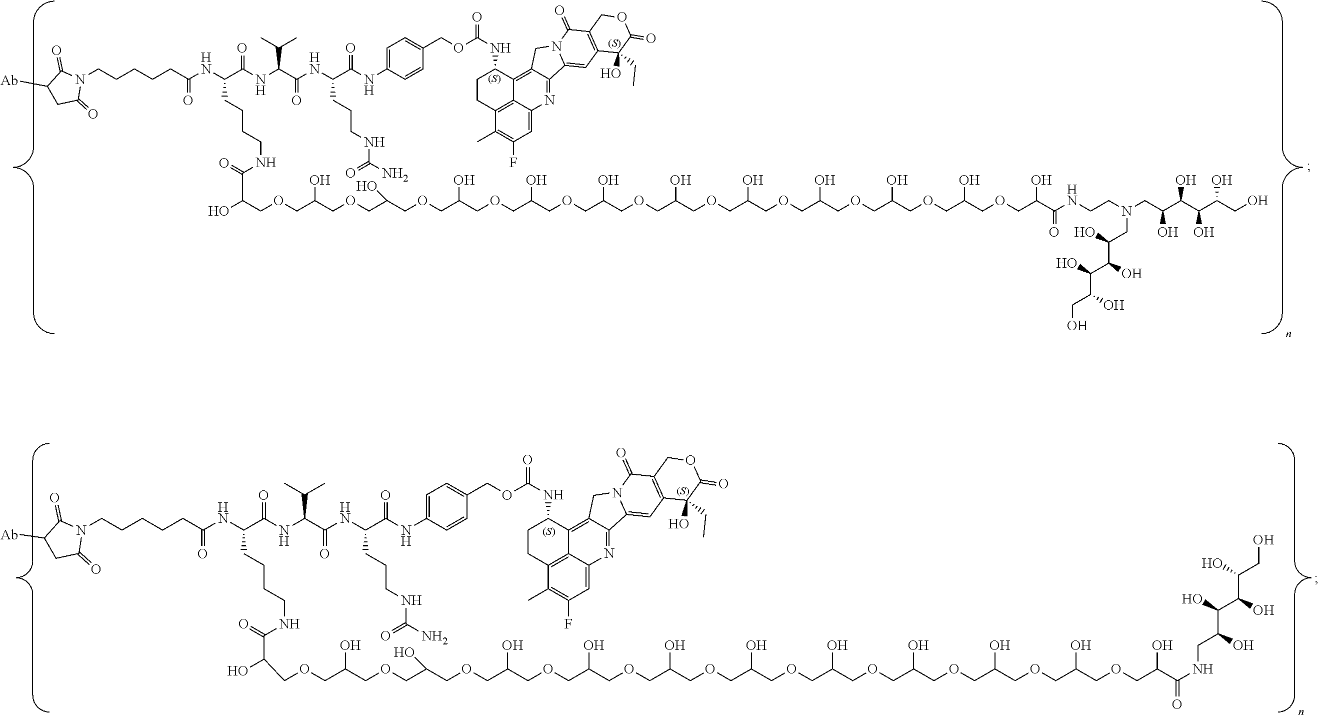

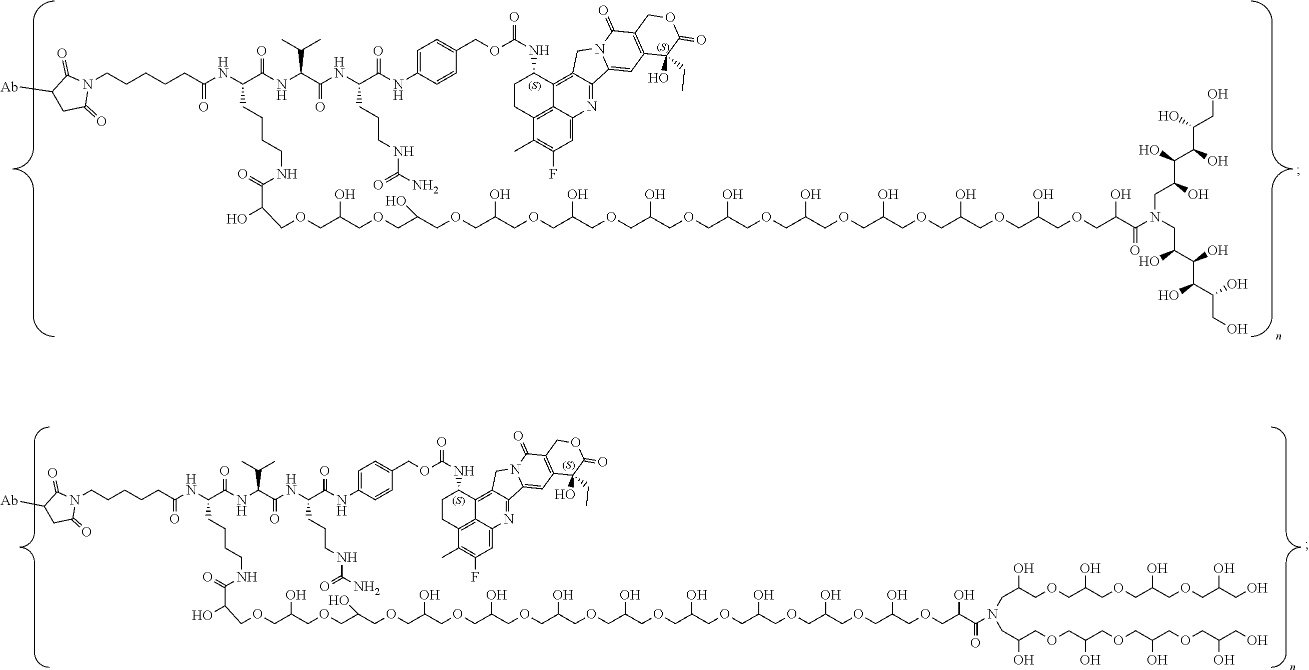

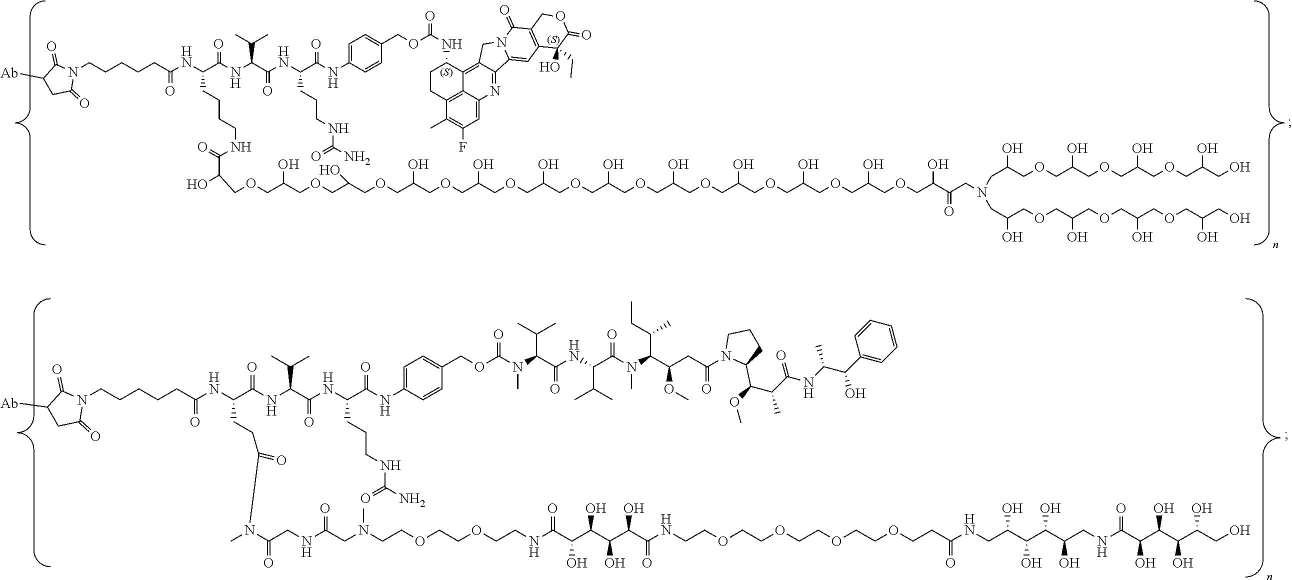

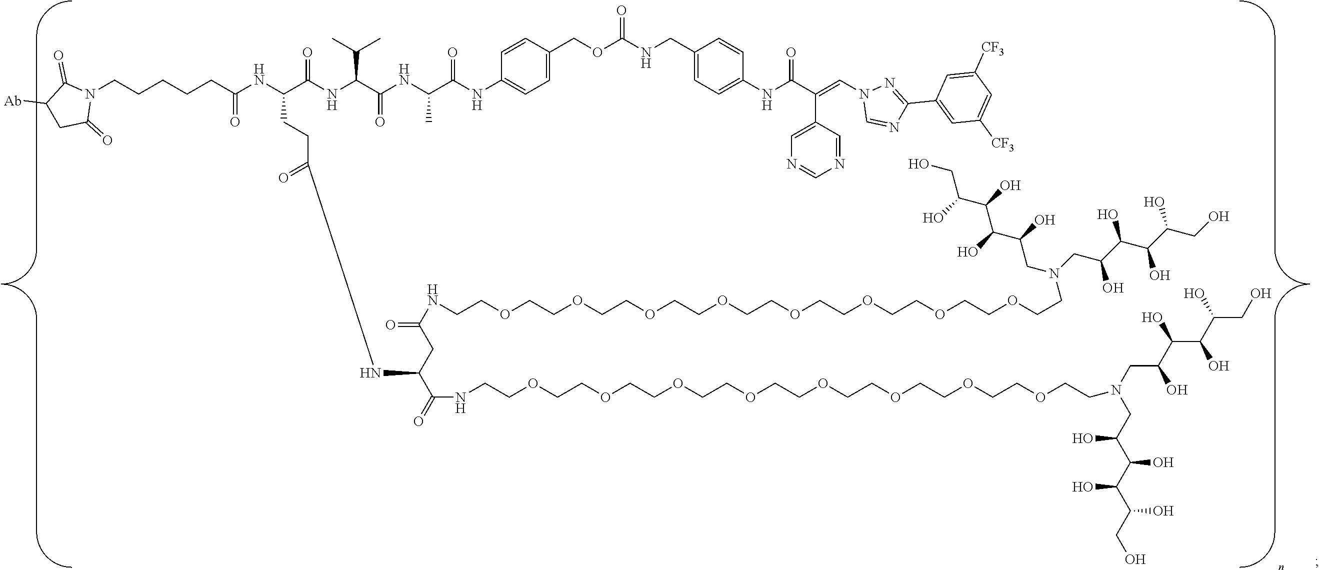

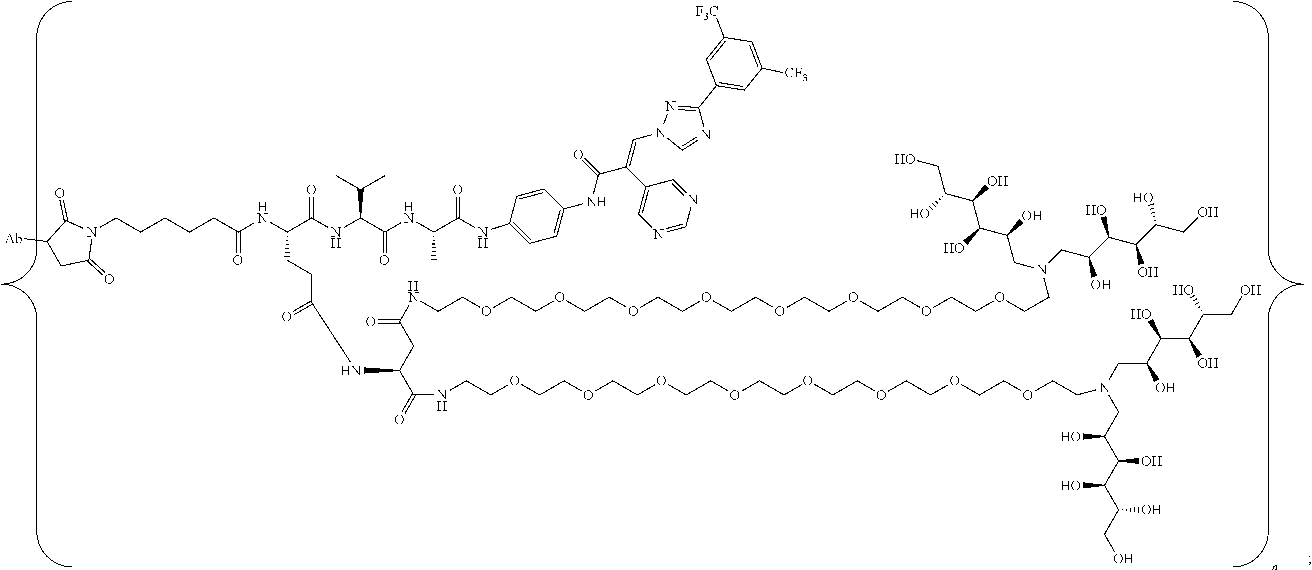

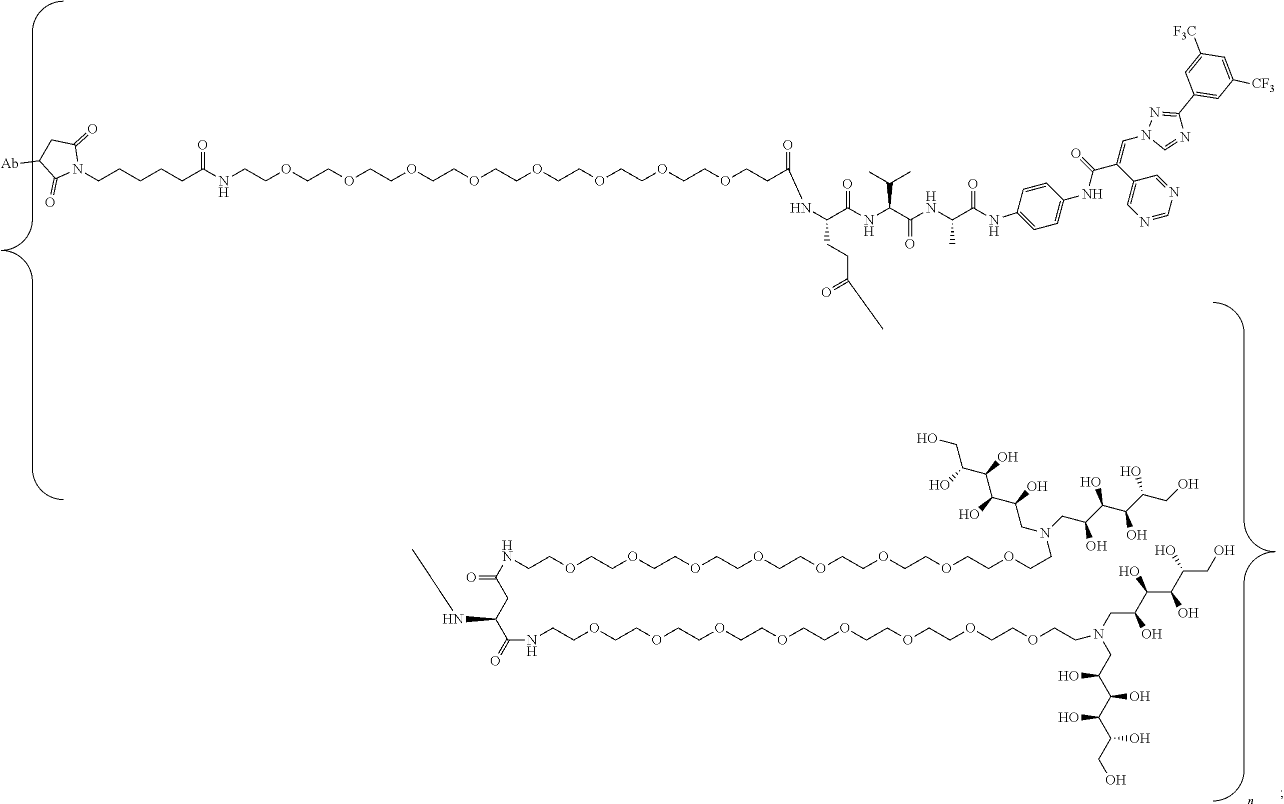

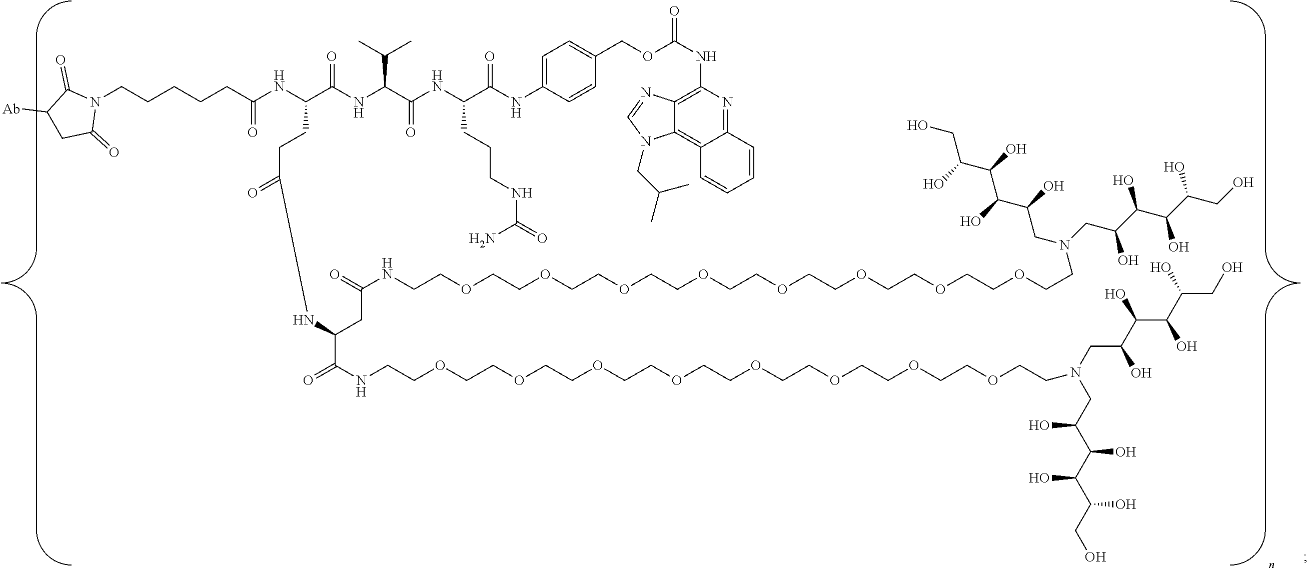

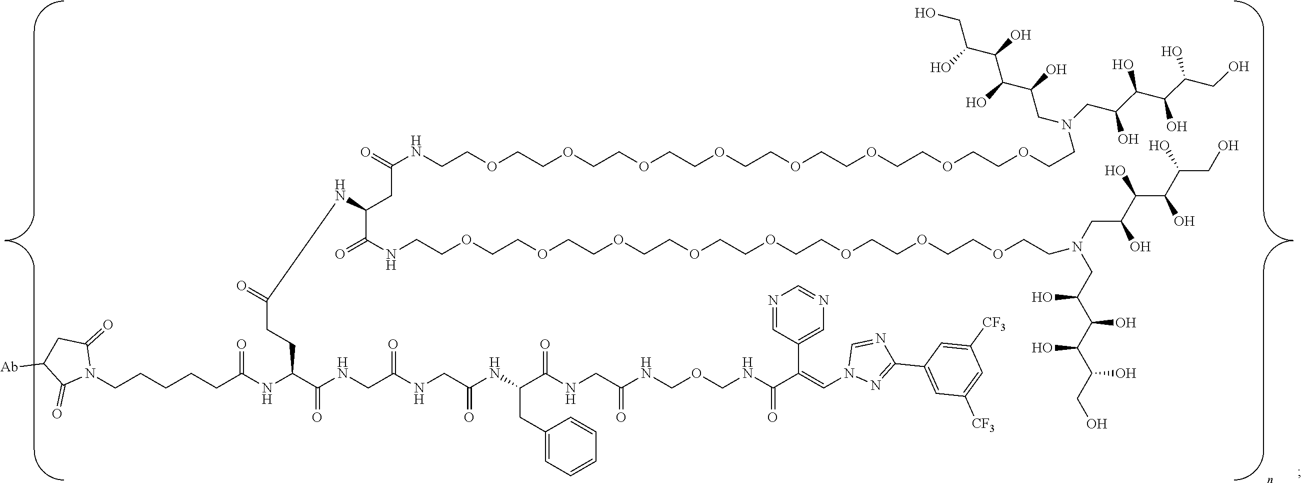

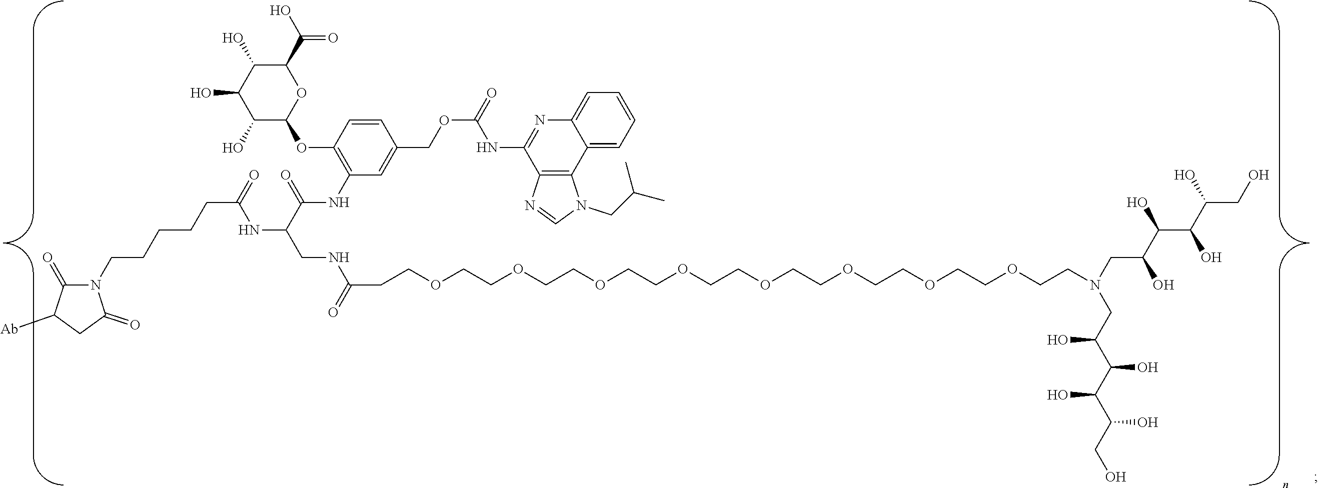

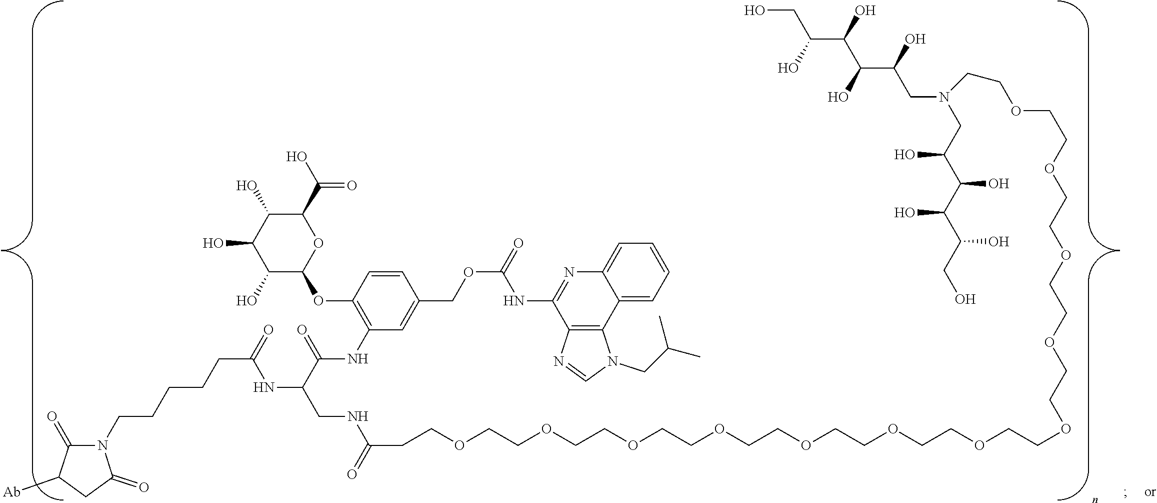

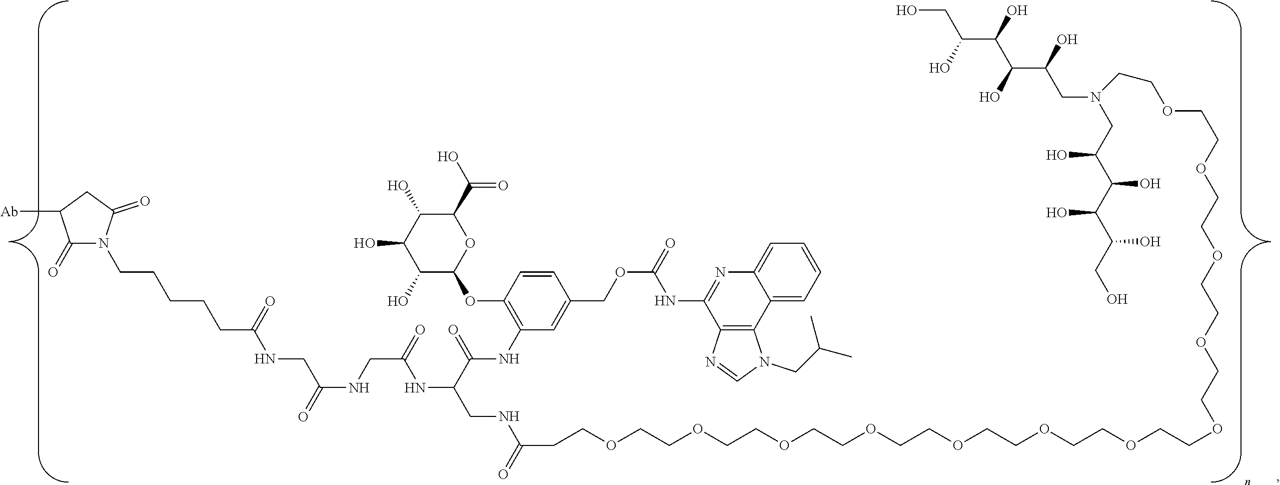

or a stereoisomer thereof.

In some embodiments, provided is a conjugate, wherein the Targeting unit binds to a target molecule.

In some embodiments, provided is a conjugate, wherein the target molecule is CD19, CD20, CD30, CD33, CD70, LIV-1, HER2, or EGFRv3. In some embodiments, provided is a conjugate, wherein the target molecule is CD19, CD20, CD30, CD33, CD70, LIV-1, or EGFRv3.

In some embodiments, provided is a conjugate, wherein the target molecule a cancer associated antigen.

In some embodiments, provided is a conjugate, wherein the target molecule is CD19, CD20, CD30, CD33, CD38, CA125, MUC-1, prostate-specific membrane antigen (PSMA), CD44 surface adhesion molecule, mesothelin (MLSN), carcinoembryonic antigen (CEA), epidermal growth factor receptor (EGFR), EGFRvIII, vascular endothelial growth factor receptor-2 (VEGFR2), HER2, high molecular weight-melanoma associated antigen (HMW-MAA), MAGE-A1, IL-13R-a2, GD2, 1p19q, ABL1, AKT1, ALK, APC, AR, ATM, BRAF, BRCA1, BRCA2, cKIT, cMET, CSF1R, CTNNB1, FGFR1, FGFR2, FLT3, GNA11, GNAQ, GNAS, HRAS, IDH1, IDH2, JAK2, KDR (VEGFR2), KRAS, MGMT, MGMT-Me, MLH1, MPL, NOTCH1, NRAS, PDGFRA, Pgp, PIK3CA, PR, PTEN, RET, RRM1, SMO, SPARC, TLE3, TOP2A, TOPO1, TP53, TS, TUBB3, VHL, CDH1, ERBB4, FBXW7, HNF1A, JAK3, NPM1, PTPN11, RB1, SMAD4, SMARCB1, STK1, MLH1, MSH2, MSH6, PMS2, ROS1, ERCC1, 5T4 (TPBG), B7-H3, CCR7, CD105, CD22, CD46, CD47, CD56, CD70, CD71, CD79b, CDH6, CLDN6, CLDN18.2, CLEC12A, DLL3, DR5, ERBB3 (HER3), EPCAM, FOLR1, IGF1R, IL2RA (CD25), IL3RA, ITGB6, LIV-1, LRRC15, mesothelin (MSLN), NaPi2b (SLC34A2), nectin-4, PTK7, ROR1, SEZ6, SLC44A4, SLITRK6, Tissue Factor (TF), TROP2 or B7-H4.

In some embodiments, provided is a conjugate, wherein the Targeting unit is an antibody, or fragment thereof, comprising rituximab (Rituxan®), trastuzumab (Herceptin®), pertuzumab (Perjeta®)), bevacizumab (Avastin®), ranibizumab (Lucentis®), cetuximab (Erbitux®), alemtuzumab (Campath®), panitumumab (Vectibix®), ibritumomab tiuxetan (Zevalin®), tositumomab (Bexxar®), ipilimumab, zalutumumab, dalotuzumab, figitumumab, ramucirumab, galiximab, farletuzumab, ocrelizumab, ofatumumab (Arzerra®), tositumumab, ibritumomab, the CD20 antibodies 2F2 (HuMax-CD20), 7D8, IgM2C6, IgG1 2C6, 11B8, B1, 2H7, LT20, iFS or AT80, daclizumab (Zenapax®), or anti-LHRH receptor antibodies including clone A9E4, F1G4, AT2G7, GNRH03, or GNRHR2.

In some embodiments, provided is a pharmaceutical composition comprising a conjugate as described herein and a pharmaceutically acceptable carrier.

In some embodiments, provided is a method of treating a subject in need thereof, comprising administering to the subject a conjugate as described herein, or a pharmaceutical composition as described herein, wherein the subject has cancer or an autoimmune disease and the conjugate binds to a target antigen associated with the cancer or autoimmune disease.

### Sugar Units (SU)

In some embodiments, Sugar units (SU) have the general formula (X):

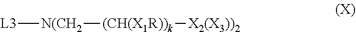

or a stereoisomer or salt thereof, wherein:
   each X₁ is independently selected from NH or O;
   each R is independently selected from hydrogen, acetyl, a monosaccharide, a disaccharide, and a polysaccharide;
   each X₂ is independently selected from CH₂ and C(O);
   each X₃ is independently selected from H, OH and OR;
   k is 1 to 10; and
L3 is a point of attachment to the remainder of the Polar group.

In some embodiments, provided is a Linker compound, wherein the Sugar unit has one of the following structures (XII) or (XIII):

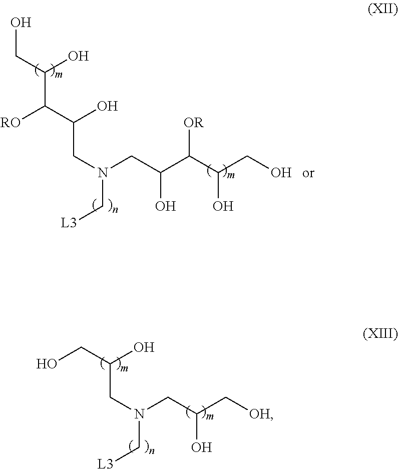

or a stereoisomer or salt thereof, wherein:
   each R is independently selected from hydrogen, a monosaccharide, a disaccharide and a polysaccharide;
   m is 1 to 8; and
   n is 0 to 4.

### Carboxyl Units

In some embodiments, a Linker comprises a Carboxyl unit. In some embodiments, a Carboxyl unit has the following general formula (XXXX):

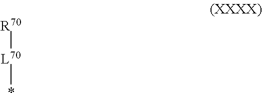

or a stereoisomer or salt thereof, wherein:
(a)
   L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment to the Amino Acid unit, or to a remainder of the Polar group;
   R⁷⁰ is ˜NR⁷¹(R⁷²—R⁷³), wherein R⁷¹ is selected from H, C₁-C₁₂ alkyl, substituted C₁-C₁₂ alkyl, or polyethylene glycol (optionally having 1 to 12 ethylene glycol subunits), R⁷² is a bond or is selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and R⁷³ is a carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide;
(b)
   L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment to the Amino Acid unit, or to a remainder of the Polar group;
   R⁷⁰ is —NR⁷¹(R⁷⁵—(R⁷³)₂), wherein R⁷¹ is selected from H, C₁-C₁₂ alkyl, substituted C₁-C₁₂ alkyl, or polyethylene glycol (optionally having 1 to 12 ethylene glycol subunits), R⁷⁵ is a branched optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl and each R⁷³ is independently carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide; or
(c)
   L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment to the Amino Acid unit, or to a remainder of the Polar group;
   R⁷⁰ is ˜N(R⁷⁴—R⁷³)(R⁷²—R⁷³), wherein R⁷² and R⁷⁴ are each independently selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and each R⁷³ is independently carboxyl or polycarboxyl, wherein comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide.

### Linker Unit

The Linkers comprise at least one Linker unit or Linker Subunit L2, each Linker unit or Linker Subunit L2 having an attachment site for at least one Drug unit (D), as further described herein. In some embodiments, a Drug unit (D) is attached to each attachment site for a Drug unit on a Linker unit or Linker Subunit L2. In various embodiments, Linker unit or Linker Subunit L2 may be a cleavable linker unit or a non-cleavable linker unit. A Linker unit or Linker Subunit L2 also has an attachment site for an Amino Acid unit (AA) or a Stretcher unit (L1).

In some embodiments, the attachment site for the Drug unit includes a linking group. A linking group can be any suitable group for connection of the at least one Drug unit that allows for release of an active Drug unit, or release of an active derivative of the linking group-Drug unit. In some embodiments, a linking group is —NH—CH₂—CH₂—CH₂—C(O)—, the Drug unit is exatecan and the released Drug unit is DXd. (See., e.g., Published US Application No. 2019/000898.)

In some embodiments, the Linker unit has from 1 to 4 attachment sites for a Drug unit. In some embodiments, the Linker unit has from 1 to 3 or 1 to 2 attachment sites for a Drug unit (D).

In some embodiments, a Linker unit or Linker Subunit L2 includes a Polar group, such as a Sugar unit, a Polymer unit, or a Carboxyl unit. In some embodiments, a Linker unit or Linker Subunit L2 does not include a Polar group, wherein an Amino Acid unit includes a Polar group. In some embodiments, both a Linker unit or Linker Subunit L2 and an Amino Acid unit (if present) include a Polar group.

In some embodiments, a Linker unit includes at least one Polar group, such as a Polymer unit. In some embodiments, the Polar group includes at least one Polymer unit and optionally a Sugar unit and/or Carboxyl unit or combinations thereof. In some embodiments, the Polymer unit is selected from an optionally substituted polyamide, a substituted polyether, or combinations thereof. In further embodiments, the Polymer unit is selected from (i) an optionally substituted polyamide comprising the formula

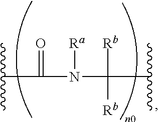

or a stereoisomer thereof, wherein each R^{a} is independently H or C₁₋₆ alkyl and each R^{b} is independently H or C₁₋₆ alkyl, and n⁰ is independently 2-26;
(ii) a substituted polyether comprising the formula

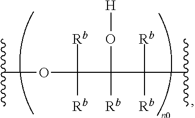

or a stereoisomer thereof, wherein each R is independently H or C₁₋₆ alkyl, and n⁰ is independently 2-26; or
(iii) combinations thereof.

In some embodiments, the Linker unit or Linker Subunit L2 is a cleavable linker unit. As used herein, the term “cleavable” refers to a metabolic process or reaction inside a cell or in the extracellular milieu, whereby the covalent attachment between a Drug unit (e.g., a cytotoxic agent) and the Linker unit or Linker Subunit L2 or portion thereof is broken, resulting in the free Drug unit, or other metabolite of the Linker unit-Drug unit or Linker Subunit L2-Drug unit dissociated from the remainder of the Linker unit or Linker Subunit L2.

In some embodiments, the Linker unit or Linker Subunit L2 includes a protease cleavable linker unit, an acid-cleavable linker unit, a disulfide linker unit, a disulfide-containing linker unit, or a disulfide-containing linker unit having a dimethyl group adjacent the disulfide bond (e.g., an SPDB linker) (see, e.g., Jain et al., Pharm. Res. 32:3526-3540 (2015); Chari et al., Cancer Res. 52:127-131 (1992); U.S. Pat. No. 5,208,020), a cleavable self-stabilizing linker (see, e.g., WO2018/031690 and WO2015/095755 and Jain et al., Pharm. Res. 32:3526-3540 (2015)), and/or a cleavable hydrophilic linker (see, e.g., WO2015/123679). In some embodiments, the Linker unit or Linker Subunit L2 includes a photolabile linker subunit. In some embodiments, the Linker unit or Linker Subunit L2 has a non-cleavable linker unit (see, e.g., WO2007/008603).

In some embodiments, the Linker unit or Linker Subunit L2 includes a glucuronide-cleavable moiety (see, e.g., US 2014/0031535).

In some embodiments, the Linker unit or Linker Subunit L2 is a cleavable linker that is cleavable under intracellular conditions, such that cleavage of or within the Linker unit or Linker Subunit L2 releases the Drug unit from Linker unit (or Linker Subunit L2) or the remainder of Linker unit in the intracellular environment. For example, in some embodiments, Linker unit or Linker Subunit L2 is cleavable by a cleaving agent that is present in the intracellular environment (e.g., within a lysosome or endosome or caveolae). As used herein, the terms “cleavable under intracellular conditions”, “intracellularly cleaved” and “intracellular cleavage” refer to a metabolic process or reaction inside a cell, whereby the covalent attachment between a Drug unit (e.g., a cytotoxic agent) and the Linker unit or Linker Subunit L2 or portion thereof is broken, resulting in the free Drug unit, or other metabolite of the Linker unit-Drug unit dissociated from the remainder of the Linker unit or Linker Subunit L2 inside the cell. The cleaved moieties of the conjugate are thus intracellular metabolites. One advantage of using intracellular proteolytic release of the Drug unit is that the activity of the Drug unit is typically attenuated when conjugated and the serum stabilities of the conjugates are typically high.

In some embodiments, a linkage between the Linker unit or Linker Subunit L2 and the Drug unit can be enzymatically cleaved by one or more enzymes, including a tumor-associated protease, to liberate the Drug unit (D). Linker unit or Linker Subunit L2 can be, for example, a peptidyl linker that is cleaved by an intracellular peptidase or protease enzyme, including, but not limited to, a lysosomal or endosomal protease (see, e.g., WO2004/010957, US20150297748, US2008/0166363, US20120328564 and US20200347075). The Linker unit or Linker Subunit L2 can be, for example, a peptidyl linker that is cleaved by an intracellular peptidase or protease enzyme, including, but not limited to, a lysosomal or endosomal protease. Intracellular protease or cleaving agents can include cathepsins B, C and D and plasmin, all of which are known to hydrolyze dipeptide drug derivatives resulting in the release of active drug inside target cells (see, e.g., Dubowchik and Walker, 1999, Pharm. Therapeutics 83:67-123). Peptidyl linkers can be cleavable by enzymes that are present in target antigen-expressing cells. For example, a peptidyl linker subunit that is cleavable by the thiol-dependent protease cathepsin-B, which is highly expressed in cancerous tissue, can be used (e.g., having a Phe-Leu, Val-Ala, Val-Cit or Gly-Phe-Leu-Gly peptide).

Typically, a Linker unit or Linker Subunit L2 has at least one amino acid or at least two amino acids that form a recognition site for a protease or other cleaving agent. In certain embodiments, the peptidyl linker is a dipeptide, tripeptide, tetrapeptide or pentapeptide. In certain embodiments, a peptidyl linker subunit can comprise only natural amino acids. In some embodiments, For example, a peptidyl linker subunit can have a Phe-Leu, Val-Ala, Val-Cit or Gly-Phe-Leu-Gly peptide. Other such cleavable linkers are described, for example, in U.S. Pat. No. 6,214,345, WO2004/010957, US20150297748, US2008/0166363, US20120328564 and US20200347075, each of which is incorporated by reference herein. In specific embodiments, the peptidyl linker that is cleavable by an intracellular protease comprises a Val-Cit peptide or a Phe-Lys peptide (see, e.g., U.S. Pat. No. 6,214,345) or Gly-Gly-Phe-Gly linker (see, e.g., US Published Application No. 2015/0297748). One advantage of using intracellular proteolytic release of the Drug unit is that the activity of the Drug unit is typically attenuated when conjugated and the serum stabilities of the conjugates are typically high. See also U.S. Pat. No. 9,345,785.

In some embodiments, a peptidyl linker subunit can comprise only non-natural amino acids. In some embodiments, a peptidyl linker subunit can comprise a natural amino acid linked to a non-natural amino acid. In some embodiments, a peptidyl linker subunit can comprise a natural amino acid linked to a D-isomer of a natural amino acid. In some embodiments, at least one amino acid of a peptidyl linker subunit is an L-amino acid. In some embodiments, at least amino acid is a D-amino acid.

In some embodiments, a Linker unit contains one or more the following: glycine and/or L-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine, that form a recognition and cleavage site for a protease or other cleaving enzyme.

In some embodiments, a peptidyl linker subunit contains one or more the following: glycine and/or L-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine, and a Polar group (including a Polymer unit(s) attached to glycine or an L-amino acid(s)). In some embodiments, a peptidyl linker subunit contains one or more the following: glycine and/or D-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine, and a Polar group (including a Polymer unit(s) attached to glycine or a D-amino acid(s)). In some embodiments, a peptidyl linker subunit contains one or more the following: glycine and/or a mixture of L-amino acids and D-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine, and a Polar group (including a Polymer unit(s) attached to glycine or an amino acid(s)).

In some embodiments, a peptidyl linker subunit contains one or more the following: glycine and/or natural L-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine and at least one Polar group, such as a Sugar unit, or a Carboxyl unit or a Polymer unit attached to glycine or an L-amino acid. In some embodiments, a peptidyl linker subunit contains one or more the following: glycine and/or D-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine and at least one Polar group, such as a Sugar unit, or a Carboxyl unit or a Polymer unit attached to glycine or an D-amino acid.

In some embodiments, an amino acid of a peptidyl linker subunit has the formula denoted below in the square brackets:

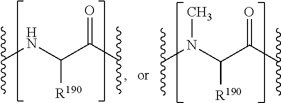

wherein R₁₉₀ is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH—CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂ 2-pyridylmethyl-3-pyridylmethyl-, 4-pyridylmethyl-, phenyl, cyclohexyl,

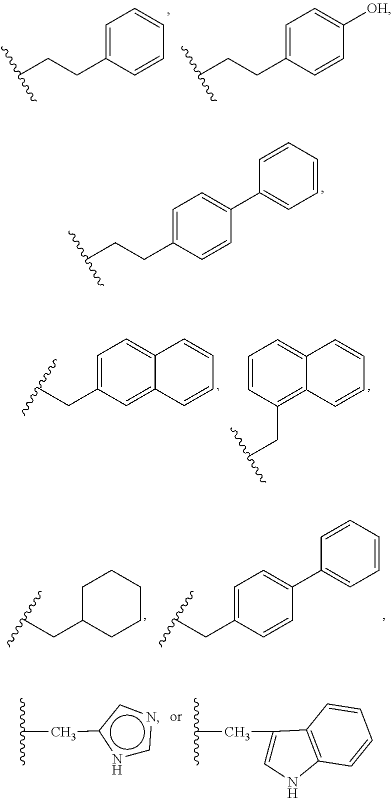

In some embodiments, a peptidyl linker subunit includes one or more of the following L-(natural) amino acids: alanine, arginine, aspartic acid, asparagine, histidine, glycine, glutamic acid, glutamine, phenylalanine, lysine, leucine, serine, tyrosine, threonine, isoleucine, tryptophan and valine; and at least one Polar group, such as a. Sugar unit., a Polymer unit, or a Carboxyl unit attached to glycine or a natural amino acid.

In some embodiments, a peptidyl linker subunit does not contain cysteine. In some embodiments, a peptidyl linker does not contain proline.

In some embodiments, a peptidyl linker subunit includes one or more of the following D-isomers of these natural amino acids: alanine, arginine, aspartic acid, asparagine, histidine, glycine, glutamic acid, glutamine, phenylalanine, lysine, leucine, serine, tyrosine, threonine, isoleucine, tryptophan and valine; and at least one Polar group, such as a Sugar unit, a Polymer unit, or Carboxyl unit attached to glycine or a D-amino acid.

In some embodiments, a peptidyl linker subunit includes one or more of the following amino acids: alanine, arginine, aspartic acid, asparagine, histidine, glycine, glutamic acid, glutamine, phenylalanine, lysine, leucine, serine, tyrosine, threonine, isoleucine, proline, tryptophan, valine, ornithine, penicillamine, β-alanine, aminoalkanoic acid, aminoalkynoic acid, amino alkanedioic acid, aminobenzoic acid, amino-heterocyclo-alkanoic acid, heterocyclo-carboxylic acid, citrulline, statine, diaminoalkanoic acid, and derivatives thereof; and at least one Polar group, such as a Sugar unit, a Polymer unit, or a Carboxyl unit attached to an amino acid(s). Illustrative of examples of derivatives of such amino acids are set forth below in the section describing the Amino Acid subunit.

In some embodiments, a peptidyl linker subunit contains a Sugar unit as part of a peptide that is cleavable. For example, a Sugar unit containing lysine or citrulline as a part of a cleavable peptide. In some embodiments, a peptidyl linker subunit contains a Carboxyl unit as part of a peptide that is cleavable. For example, a Carboxyl unit containing lysine or citrulline as a part of a cleavable peptide.

In some embodiments, a cleavable linker subunit is pH-sensitive, i.e., sensitive to hydrolysis at certain pH values. Typically, a pH-sensitive linker subunit is hydrolyzable under acidic conditions. For example, an acid-labile linker subunit that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; and 5,622,929; Dubowchik and Walker, 1999, Pharm. Therapeutics 83:67-123; Neville et al., 1989, Biol. Chem. 264:14653-14661.) Such linker subunits are relatively stable under neutral pH conditions, such as those in the blood, but are unstable at below pH 5.5 or 5.0, the approximate pH of the lysosome. In certain embodiments, a hydrolyzable linker unit is a thioether linker (such as, for example, a thioether attached to the Drug unit via an acylhydrazone bond (see, e.g., U.S. Pat. No. 5,622,929)).

In some embodiments, a Linker unit or Linker Subunit L2 is cleavable under reducing conditions (e.g., a disulfide linker subunit). A variety of disulfide linkers are known, including, for example, those that can be formed using SATA (N-succinimidyl-5-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene)-, SPDB and SMPT (see, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

In some embodiments, a Linker unit or Linker Subunit L2 is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12). In some embodiments, the Linker unit or Linker Subunit L2 is not cleavable, such as a maleimidocaproyl linker, and the Drug unit is released by metabolic degradation of the Drug-Linker. (See, e.g., U.S. Publication No. 2005/0238649.)

In some embodiments, a Linker unit or Linker Subunit L2 is not substantially sensitive to the extracellular environment. As used herein, “not substantially sensitive to the extracellular environment,” in the context of a Linker unit or Linker Subunit L2, means that no more than about 20%, typically no more than about 15%, more typically no more than about 10%, and even more typically no more than about 5%, no more than about 3%, or no more than about 1% of the Linker unit or Linker Subunit L2 in a sample of conjugate, are cleaved when the conjugate is present in an extracellular environment (e.g., in plasma). Whether a Linker unit or Linker Subunit L2 is not substantially sensitive to the extracellular environment can be determined, for example, by incubating independently with plasma both (a) the conjugate (the “conjugate sample”) and (b) an equal molar amount of unconjugated Targeting unit or Drug unit (the “control sample”) for a predetermined time period (e.g., 2, 4, 8, 16, or 24 hours) and then comparing the amount of unconjugated Targeting unit or Drug unit present in the conjugate sample with that present in control sample, as measured, for example, by high performance liquid chromatography.

In some embodiments, a Linker or Linker Subunit L2 promotes cellular internalization. In some embodiments, a Linker or Linker Subunit L2 promotes cellular internalization when conjugated to the Drug unit such as a cytotoxic agent (i.e., in the milieu of the Linker-Drug unit moiety of a conjugate as described herein). In yet other embodiments, a Linker or Linker Subunit L2 promotes cellular internalization when conjugated to both the Drug unit and the Targeting unit (i.e., in the milieu of a conjugate as described herein).

A variety of Linker units or Linker Subunits L2 that can be used with the present compositions and methods are described in, for example, WO 2004010957. In some embodiments, a Linker unit or Linker Subunit L2 includes a protease cleavable linker comprising a thiol-reactive spacer and a dipeptide (e.g., maleimidyl caproyl valine alanine). In some embodiments, a Linker unit or Linker Subunit L2 includes protease cleavable linker comprising a thiol-reactive maleimidocaproyl spacer or Stretcher, an amino acid or peptide and a self-immolative group. In some embodiments, a Linker unit or Linker Subunit L2 includes protease cleavable linker comprising a thiol-reactive maleimidocaproyl spacer, a valine-citrulline dipeptide, and a p-amino-benzyloxycarbonyl self immolative group.

In some embodiments, a Linker unit or Linker Subunit L2 includes an acid cleavable linker such as a hydrazine linker or a quaternary ammonium linker (see, e.g., WO2017/096311 and WO2016/040684.)

In some embodiments, a Linker unit or Linker Subunit L2 includes a self-stabilizing moiety comprising a maleimide group as described in WO2013/173337.

In some embodiments, a Linker unit or Linker Subunit L2 includes a hydrophilic linker, such as, for example, the hydrophilic peptides in WO2015/123679 and the sugar alcohol polymer-based linkers disclosed in WO2013/012961 and WO2019/213046.

In other embodiments, a Linker unit or Linker Subunit L2 may be made using a variety of bifunctional protein coupling agents such as N-succinimidyl-3-(2-pyridyldithio) propionate (SPDP), succinimidyl-4-(N-maleimidomethyl) cyclohexane-1-carboxyl (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Chelating agents for conjugation of a radionucleotide(s) have been described in, for example WO94/11026.

In some embodiments, Linker units or Linker Subunit L2, can be prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A.).

### Amino Acid (AA) Unit

The Linkers optionally include an Amino Acid unit (AA). When present in a Linker, an Amino Acid unit connects a Stretcher unit (L1) to a Linker unit. When s of AA is 0, the Amino Acid unit is absent (e.g., in any of Formulae I to IV). In some embodiments, an Amino Acid unit includes from 0 to 12 subunits. Each subunit of the Amino Acid unit is selected from a natural or non-natural alpha, beta or gamma amino acid or a Polar group, such as a Sugar unit (SU), a Polymer unit, or a Carboxyl unit attached to a subunit of the Amino Acid unit.

In some embodiments, an Amino acid unit is an amino acid or a dipeptide, tripeptide, tetrapeptide, pentapeptide, hexapeptide, heptapeptide, octapeptide, nonapeptide, decapeptide, undecapeptide or dodecapeptide, in which one or more of the subunits is optionally modified to form a Polar group, such as a Sugar unit, a Polymer unit, or a Carboxyl unit.

In some embodiments, the subunits of the Amino Acid unit are selected from glycine and/or L-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine, and Polar groups (including Polymer units attached to glycine or an L-amino acid). In some embodiments, the subunits of the Amino Acid unit are selected from glycine and/or D-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine, and Polar groups. In some embodiments, the subunits of the Amino Acid unit are selected from glycine and/or a mixture of L-amino acids and D-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine, and Polar groups (including Polymer units attached to glycine or an D-amino acid).

In some embodiments, the subunits of the Amino Acid unit are selected from glycine and/or natural L-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine and at least one Polar group, such as a Sugar unit, a Polymer unit, or a Carboxyl unit attached to a glycine or a L-amino acid. In some embodiments, the subunits of the Amino Acid unit are selected from glycine and/or D-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine and at least one Polar group, such as a Sugar unit, a Polymer unit, or a Carboxyl unit attached to a glycine or a D-amino acid.

In some embodiments, a subunit of the Amino acid unit independently has the formula denoted below in the square brackets:

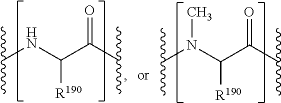

wherein R₁₉₀ is hydrogen methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH—CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂ 2-pyridylmethyl-3-pyridylmethyl-, 4-pyridylmethyl-, phenyl, cyclohexyl,

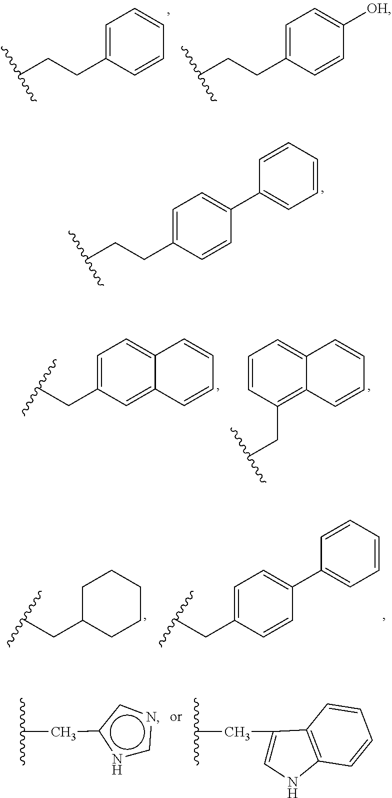

In some embodiments, each subunit of the Amino Acid unit is independently selected from the group consisting of the following L-(natural) amino acids: alanine, arginine, aspartic acid, asparagine, histidine, glycine, glutamic acid, glutamine, phenylalanine, lysine, leucine, serine, tyrosine, threonine, isoleucine, tryptophan and valine; and at least one Polar group, such as a Sugar unit, a Polymer unit, or a Carboxyl unit attached to a natural amino acid.

In some embodiments, a subunit of the Amino acid unit is not cysteine. In some embodiments, a subunit of the Amino Acid unit is not proline.

In some embodiments, each subunit of the Amino Acid unit is independently selected from the group consisting of the following D-isomers of these natural amino acids: alanine, arginine, aspartic acid, asparagine, histidine, glycine, glutamic acid, glutamine, phenylalanine, lysine, leucine, serine, tyrosine, threonine, isoleucine, tryptophan and valine; and at least one Polar group, such as a Sugar unit, a Polymer unit, or a Carboxyl unit attached to glycine or an L-amino acid.

In some embodiments, each subunit of the Amino Acid unit is independently selected from alanine, arginine, aspartic acid, asparagine, histidine, glycine, glutamic acid, glutamine, phenylalanine, lysine, leucine, serine, tyrosine, threonine, isoleucine, proline, tryptophan, valine, ornithine, penicillamine, β-alanine, aminoalkanoic acid, aminoalkynoic acid, amino alkanedioic acid, aminobenzoic acid, amino-heterocyclo-alkanoic acid, heterocyclo-carboxylic acid, citrulline, statine, diaminoalkanoic acid, and derivatives thereof; and at least one Polar group, such as a Sugar unit, a Polymer unit, or a Carboxyl unit attached to one of the subunits.

Illustrative of examples of alanine and derivatives thereof include but are not limited to: alanine (Ala), N-alkyl-alanine, dehydro-alanine, 4-thiazolylalanine, 2-pyridylalanine, 3-pyridylalanine, 4-pyridylalanine, β-(1-naphthyl)-alanine, β-(2-naphthyl)-alanine, α-aminobutyric acid, β-chloro-alanine, β-cyano-alanine, β-cyclopentyl-alanine, β-cyclohexyl-alanine, β-iodo-alanine, β-cyclopentenyl-alanine, β-tBu-alanine, β-cyclopropyl-alanine, β-diphenyl-alanine, β-fluoro-alanine, β-piperazinyl-alanine with the piperazine ring protected or not, β-(2-quinolyl)-alanine, β-(1,2,4-triazol-1-yl)-alanine, β-ureido-alanine, H-β-(3-benzothienyl)-Ala-OH, and H-β-(2-thienyl)-Ala-OH.

Illustrative of examples of arginine and derivatives thereof include but are not limited to: arginine (Arg), N-alkyl-arginine, H-Arg(Me)-OH, H-Arg(NH₂)—OH, H-Arg(NO₂)—OH, H-Arg(Ac)₂-OH, H-Arg(Me)₂-OH (asymmetrical), H-Arg(Me)₂-OH (symmetrical), 2-amino-4-(2′-hydroxyguanidino)-butyric acid (N-ω-hydroxy-nor-arginine) and homoarginine.

Illustrative of examples of aspartic acid and derivatives thereof include but are not limited to: aspartic acid (Asp), N-alkyl-aspartic acid, and H-Asp(OtBu)-OH.

Illustrative of examples of asparagine and derivatives thereof include but are not limited to: asparagine (Asn), N-alkyl-asparagine, and isoasparagine (H-Asp-NH₂).

Illustrative of examples of cysteine (Cys) derivatives (containing no free SH group) thereof include but are not limited to: H-Cys(Acm)-OH, H-Cys(Trt)-OH, H-Cys(tBu)-OH, H-Cys(Bzl)-OH, H-Cys(Et)-OH, H-Cys(SO₃H)—OH, H-Cys(aminoethyl)-OH, H-Cys(carbamoyl)-OH, H-Cys(phenyl)-OH, H-Cys(Boc)-OH, and H-Cys(hydroxyethyl)-OH.

Illustrative of examples of histidine and derivatives thereof include but are not limited to: histidine (His), N-alkyl-histidine, H-His(Boc)-OH, H-His(Bzl)-OH, H-HBs(I-Me)-OH, H-His(1-Tos)-OH, H-2,5-diiodo-His-OH, and H-His(3-Me)-OH.

Illustrative of examples of glycine and derivatives thereof include but are not limited to: glycine (Gly), N-alkyl-glycine, H-propargylglycine

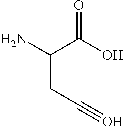

CH), α-aminoglycine (protected or not), β-cyclopropyl-glycine, cyclopentyl-glycine, cyclohexyl-glycine, α-allylglycine, t-Butyl-glycine, neopentylglycine, and phenylglycine.

Illustrative of examples of glutamic acid and derivatives thereof include but are not limited to: glutamic acid (GIu), N-alkyl-glutamic acid, H-GIu(OtBu)-OH, H-γ-hydroxy-Glu-OH, H-γ-methylene-Glu-OH, H-γ-carboxy-Glu(OtBu)₂-OH, and pyroglutamic acid.

Illustrative of examples of glutamine and derivatives thereof include but are not limited to: glutamine (GIn), N-alkyl-glutamine, isoglutamine (H-GIu-NH₂), H-GIn(Trt)-OH, and H-Gln(isopropyl)-OH.

Illustrative of examples of phenylalanine and derivatives thereof include but are not limited to: phenylalanine (Phe), N-alkyl-phenylalanine, H-p-amino-Phe-OH, H-p-amino-Phe(Z)-OH, H-p-bromo-Phe-OH, H-p-Benzyl-Phe-OH, H-p-tBu-Phe-OH, H-p-carboxy-Phe(OtBu)-OH, H-p-carboxy-Phe-OH, H-p-cyano-Phe-OH, H-p-fluoro-Phe-OH, H-3,4-dichloro-Phe-OH, H-p-iodo-Phe-OH, H-p-nitro-Phe-OH, H-p-methyl-Phe-OH, H-pentafluoro-Phe-OH, H-m-fluoro-Phe-OH, H-α-Me-Phe-OH, H-4-phenyl-Phe-OH, homophenylalanine, chloro-phenylalanine and β-homophenylalanine.

Illustrative of examples of lysine and derivatives thereof include but are not limited to: lysine (Lys), N-alkyl-lysine, H-Lys(Boc)-OH, H-Lys(Ac)-OH, H-Lys(Formyl)-OH, H-Lys(Me)₂-OH, H-Lys(nicotinoyl)-OH, H-Lys(Me)₃-OH, H-trans-4,5-dehydro-Lys-OH, H-Lys(Aloc)-OH, H—H-δ-hydroxy-Lys-OH, H-δ-hydroxy-Lys(Boc)-OH, H-Lys(acetamidoyl)-OH, and H-Lys(isopropyl)-OH Illustrative of examples of leucine and derivatives thereof include but are not limited to: leucine (Leu), N-alkyl-leucine, 4,5-dehydroleucine, H-α-Me-Leu-OH, homoleucine, norleucine, and t-leucine.

Illustrative of examples of methionine and derivatives thereof include but are not limited to: methionine (Met), H-Met(O)-OH, and H-Met(O)₂—OH.

Illustrative of examples of serine and derivatives thereof include but are not limited to: serine (Ser), N-alkyl-serine, H-Ser(Ac)-OH, H-Ser(tBu)-OH, H-Ser(Bzl)-OH, H-Ser(p-chloro-Bzl)-OH, H-β-(3,4-dihydroxyphenyl)-Ser-OH, H-β-(2-thienyl)-Ser-OH, isoserine N-alkyl-isoserine, and 3-phenyliso serine.

Illustrative of examples of tyrosine and derivatives thereof include but are not limited to: tyrosine (Tyr), N-alkyl-tyrosine, H-3,5-dinitro-Tyr-OH, H-3-amino-Tyr-OH, H-3,5-dibromo-Tyr-OH, H-3,5-diiodo-Tyr-OH, H-Tyr(Me)-OH, H-Tyr(tBu)-OH, H-Tyr(Boc)-OH, H-Tyr(Bzl)-OH, H-Tyr(Et)-OH, H-3-iodo-Tyr-OH, and H-3-nitro-Tyr-OH.

Illustrative of examples of threonine and derivatives thereof include but are not limited to: threonine (Thr), N-alkyl-threonine, allo-threonine, H-Thr(Ac)-OH, H-Thr(tBu)-OH, and H-Thr(Bzl)-OH.

Illustrative of examples of isoleucine and derivatives thereof include but are not limited to: isoleucine (He), N-alkyl-isoleucine, allo-isoleucine, and norleucine.

Illustrative of examples of tryptophan and derivatives thereof include but are not limited to: tryptophan (Tip), N-alkyl-tryptophan, H-5-Me-Trp-OH, H-5-hydroxy-Trp-OH, H-4-Me-Trp-OH, H-α-Me-Trp-OH, H-Trp(Boc)-OH, H-Trp(Formyl)-OH, and H-Trp(Mesitylene-2-sulfonyl)-OH.

Illustrative of examples of proline and derivatives thereof include but are not limited to: proline (Pro), N-alkyl-proline, homoproline, thioproline, hydroxyproline (H-Hyp-OH), H-Hyp(tBu)-OH, H-Hyp(Bzl)-OH, H-3,4-dehydro-Pro-OH, 4-keto-proline, α-Me-Pro-OH, and H-4-fluoro-Pro-OH.

Illustrative of examples of valine and derivatives thereof include but are not limited to: valine (Val), N-alkyl-valine, H-α-Me-Val-OH, and norvaline.

Illustrative of examples of ornithine and derivatives thereof include but are not limited to: ornithine, N-alkyl-ornithine, H-Om(Boc)-OH, H-Om(Z)-OH, H-α-difluoro-Me-Orn-OH (Eflomitine), and H-Om(Aloc)-OH.

Illustrative of examples of penicillamine and derivatives thereof include but are not limited to: penicillamine, H-penicillamme(Acm)-OH (H-β,β-dimethylcys(Acm)-OH) and N-alkyl-penicillamine.

Illustrative of examples of β-alanine and derivatives thereof include but are not limited to: β-alanine, N-alkyl-β-alanine, and dehydro-alanine.

Illustrative of examples of an aminoalkanoic acid and derivatives thereof include but are not limited to: N-alkylaminoalkanoic acid, aminobutyric acid, 4-(neopentyloxysulfonyl)-aminobutyric acid, ε-aminocaproic acid, α-aminoisobutyric acid, piperidylacetic acid, 3-ammopropionic acid, 3-amino-3-(3-pyridyl)-propionic acid, and 5-aminopentanioic acid (amino valeric acid).

Illustrative of examples of an aminoalkynoic acid and derivatives thereof include but are not limited to: N-alkylaminoalkynoic acid, 6-amino-4-hexynoic acid, 6-(Boc-amino)-4-hexynoic acid.

Illustrative of examples of an aminoalkanedioic acid and derivatives thereof include but are not limited to: N-alkylaminoalkanedioic acid, 2-aminohexanedioic acid, 2-aminoheptanedioic acid, 2-aminooctanedioic acid (H-Asu-OH).

Illustrative of examples of an aminobenzoic acid and derivatives thereof include but are not limited to: N-alkylaminobenzoic acid, 2-aminobenzoic acid, 3-aminobenzoic acid, and 4-aminobenzoic acid.

Illustrative of examples of an amino-heterocyclo-alkanoic acid and derivatives thereof include but are not limited to: N-alkylamino-heterocyclo-alkanoic acids, 4-amino-1-methyl-1H-imidazol-2-carboxylic acid, 4-amino-1-methyl-1H-pyrrole-2-carboxylic acid, 4-amino-piperidine-4-carboxylic acid (H-Pip-OH; 1-protected or not), 3-amino-3-(3-pyridyl)-propionic acid.

Illustrative of examples of a heterocyclo-carboxylic acid and derivatives thereof include but are not limited to: azetidine-2-carboxylic acid, azetidine-3-carboxylic acid, piperidine-4-carboxylic acid, and thiazolidine-4-carboxylic acid.

Illustrative of examples of citrulline and derivatives thereof include but are not limited to: citrulline (cit), N-alkyl-citrulline, thio citrulline, S-methyl-thiocitrulline, and homocitrulline.

Illustrative of examples of statine and derivatives thereof include but are not limited to: statine, N-alkyl-statine, cyclohexylstatine, and phenylstatilie.

Illustrative of examples of diaminoalkanoic acid (Dab) and derivatives thereof include but are not limited to: N-alkyl-diamino-alkanoic acids, N,N-dialkylamino-alkanoic acids, α,γ-diaminobutyric acid (H-Dab-OH), H-Dab(Aloc)-OH, H-Dab(Boc)-OH, H-Dab(Z)-OH, α,β-diaminopropionic acid and its side-chain protected versions.

In some embodiments, an Amino Acid unit may be terminated with a capping group, such as a straight chain or branched alkyl group, or a polyethylene chain (from 1 to 30 subunits) or a Polymer unit.

Exemplary embodiments of an Amino Acid unit include the following, wherein SU is a Sugar unit, POLY is a Polymer unit and CU is a Carboxyl unit:

In some embodiments, an Amino Acid unit comprises SU.

In some embodiments, an Amino Acid unit comprises SU-Lys-SU.

In some embodiments, an Amino Acid unit comprises SU-Lys-SU-tert-butyl.

In some embodiments, an Amino Acid unit comprises SU-Lys.

In some embodiments, an Amino Acid unit comprises Lys-SU.

In some embodiments, an Amino Acid unit comprises Lys-SU-Lys(POLY).

In some embodiments, an Amino Acid unit comprises SU-Lys(POLY)-SU.

In some embodiments, an Amino Acid unit comprises SU-Glu-SU.

In some embodiments, an Amino Acid unit comprises Lys(POLY).

In some embodiments, an Amino Acid unit comprises Lys(POLY)-Lys(POLY)

In some embodiments, an Amino Acid unit comprises CU.

In some embodiments, an Amino Acid unit comprises CU—CU.

In some embodiments an Amino Acid Unit is present and is linked to a peptide of a Linker Subunit L2 via a peptide bond. In some embodiments, such an Amino Acid unit-Linker Subunit L2 comprises SU-Val-Cit˜, wherein the wavy line indicates a bond to the remainder of Linker Subunit L2 or to a Drug unit. In some embodiments, such an Amino Acid unit-Linker Subunit L2 comprises SU-Val-Ala˜, wherein the wavy line indicates a bond to the remainder of Linker Subunit L2 or to a Drug unit. In some embodiments, such an Amino Acid unit-Linker Subunit L2 comprises SU-Val-Lys˜, wherein the wavy line indicates a bond to the remainder of Linker Subunit L2 or to a Drug unit. In some embodiments, such an Amino Acid unit-Linker Subunit L2 comprises SU-Gly-Gly-Phe-Gly˜, wherein the wavy line indicates a bond to the remainder of Linker Subunit L2 or to a Drug unit.

In some embodiments, such an Amino Acid unit-Linker Subunit L2 comprises Val-Lys(POLY)˜, wherein the wavy line indicates a bond to the remainder of Linker Subunit L2 or to a Drug unit. In some embodiments, such an Amino Acid unit-Linker Subunit L2 comprises Val-Cit(POLY)˜, wherein the wavy line indicates a bond to the remainder of Linker Subunit L2 or to a Drug unit. In some embodiments, such an Amino Acid unit-Linker Subunit L2 comprises Lys(POLY)-Val-Cit˜, wherein the wavy line indicates a bond to the remainder of Linker Subunit L2 or to a Drug unit. In some embodiments, such an Amino Acid unit-Linker Subunit L2 comprises Lys(POLY)-Gly-Gly-Phe-Gly˜, wherein the wavy line indicates a bond to the remainder of Linker Subunit L2 or to a Drug unit.

In some embodiments, such an Amino Acid unit-Linker Subunit L2 comprises CU-Val-Cit˜, wherein the wavy line indicates a bond to the remainder of Linker Subunit L2 or to a Drug unit. In some embodiments, such an Amino Acid unit-Linker Subunit L2 comprises CU-Val-Lys˜, wherein the wavy line indicates a bond to the remainder of Linker Subunit L2 or to a Drug unit. In some embodiments, such an Amino Acid unit-Linker Subunit L2 comprises CU-Val-Ala˜, wherein the wavy line indicates a bond to the remainder of Linker Subunit L2 or to a Drug unit. In some embodiments, such an Amino Acid unit-Linker Subunit L2 comprises Val-CU˜, wherein the wavy line indicates a bond to the remainder of Linker Subunit L2 or to a Drug unit, and wherein CU comprises a Lysine residue. In some embodiments, such an Amino Acid unit-Linker Subunit L2 comprises CU-Gly-Gly-Phe-Gly˜, wherein the wavy line indicates a bond to the remainder of Linker Subunit L2 or to a Drug unit.

In some embodiments, the Amino Acid unit is present and is attached to Linker Subunit L2 by a non-peptidic bond. In some embodiments, the Amino Acid unit is attached to Linker Subunit L2 by a peptidic linking group such as a C₁-C₁₀ alkylene, C₂-C₁₀ alkenylene, C₂-C₁₀ alkynylene, or polyethylene glycol.

In some embodiments, provided is a Linker intermediate or Linker, wherein L2 or AA-L2 has one of the following structures:

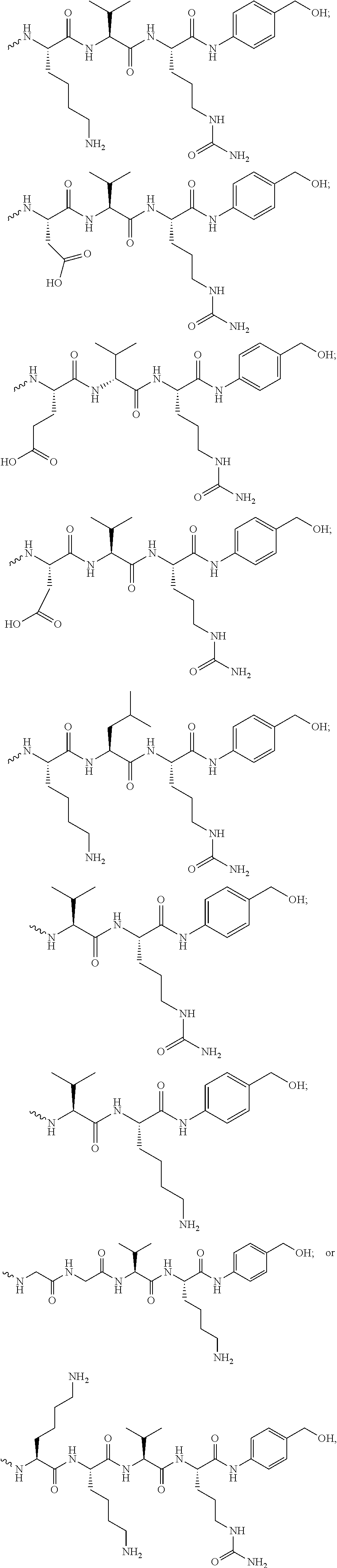

wherein the wavy line on the amino group indicates an attachment site for a Stretcher unit, and the Drug unit is attached to the benzyl alcohol.

### Stretcher Unit (L1)

The Stretcher unit (L1) is capable of linking a Targeting unit to an Amino Acid unit (AA) or to a Linker Subunit L2. A Stretcher unit has a functional group that can form a bond with a functional group of a Targeting unit. In some embodiments of the Linker, the Stretcher unit is attached to an Amino Acid unit, which is attached to a Linker Subunit L2 (i.e., when s of AA is 1; see e.g., Formulae (I) to (IV)). In some embodiments, a Stretcher unit is attached to a Linker Subunit L2 (i.e., when s of AA is 0; see e.g., Formulae (I) to (IV)). In some embodiments, a Stretcher unit is attached to an Amino Acid unit-Linker Subunit L2 after the Amino Acid unit-Linker Subunit L2 is formed. In some embodiments, a Stretcher unit is attached to an Amino Acid unit-Linker Subunit L2-Drug unit after the Amino Acid unit-Linker Subunit L2-Drug unit is formed. In some embodiments, a Stretcher unit is attached to a Linker Subunit L2-Drug unit after the Linker Subunit L2-Drug unit is formed.

A functional group of the Stretcher unit for attachment to a Targeting unit may include, for example, maleimide, haloacetamide, sulfhydryl group, NHS ester, aldehyde, ketone, carbonyl, hydrazide, hydroxylamine, amine, amino, hydrazine, thiosemicarbazone, hydrazine carboxyl, or arylhydrazide.

Functional groups that can be present on a Targeting unit, either naturally or via chemical manipulation include, but are not limited to, sulfhydryl (—SH), amino, hydroxyl, carboxy, the anomeric hydroxyl group of a carbohydrate, and carboxyl groups. In one aspect, the Targeting unit's functional groups are sulfhydryl and amino. Sulfhydryl groups can be generated by reduction of an intramolecular disulfide bond of a Targeting unit. Alternatively, sulfhydryl groups can be generated by reaction of an amino group of a lysine moiety of a Targeting unit using 2-iminothiolane (Traut's reagent) or another sulfhydryl generating reagent.

In some embodiments, the Stretcher unit forms a bond with a sulfur atom of a Targeting unit via a maleimide group of the Stretcher unit. The sulfur atom can be derived from, for example, a sulfhydryl group of a Targeting unit (e.g., a thiol group of an interchain disulfide bond). Representative Stretcher units of this embodiment are depicted in the following Formulas 100 and 101, wherein L is a Targeting unit and the wavy line indicates an attachment site for an Amino Acid unit or to a Linker Subunit L2:

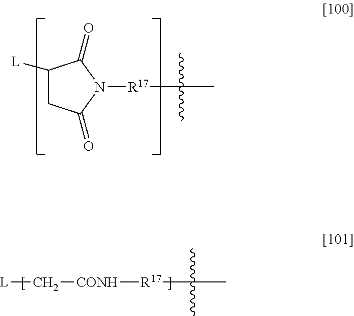

In some embodiments, provided is a Linker, wherein the Stretcher unit is selected from the following:

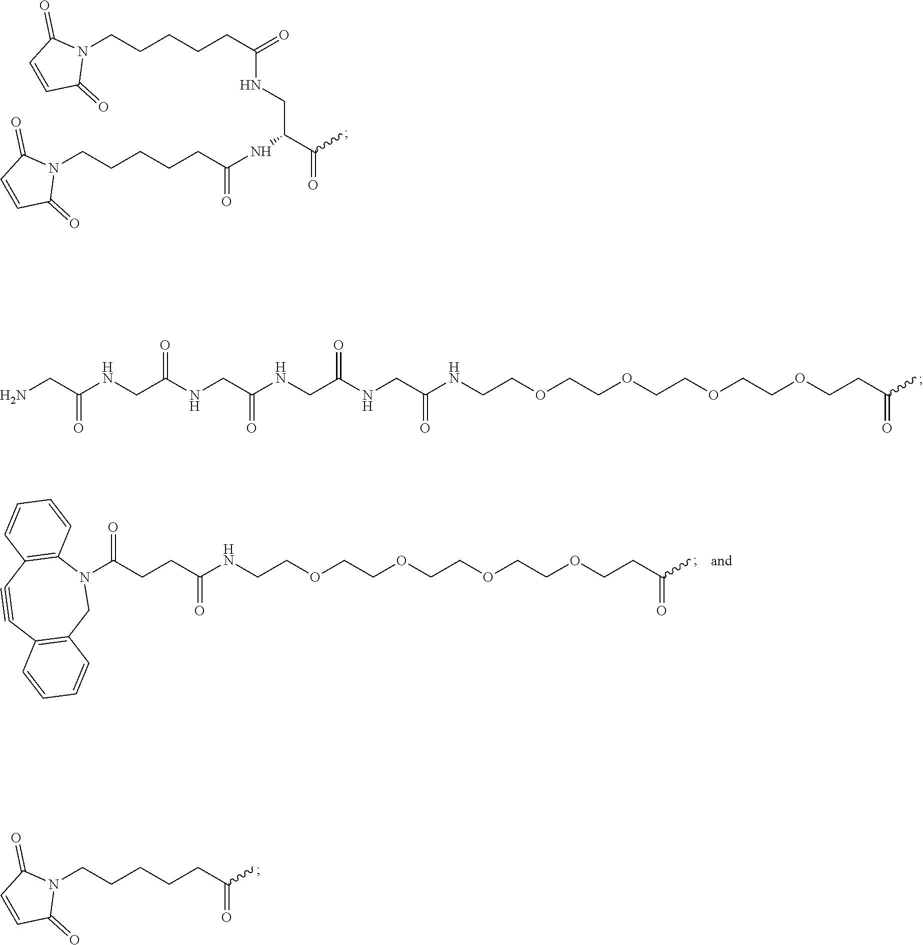

wherein the wavy line [symbol] indicates an attachment site of the Stretcher unit to an Amino Acid unit.

In formulas 100 and 101, R¹⁷ is —C₁-C₁₀ alkylene-, —C₁-C₁₀ heteroalkylene-, —C₃-C₈ carbocyclo-, —O—(C₁-C₈ alkylene)-, —(CH₂—O—CH₂)_{b}—C₁-C₈ alkylene- (where b is 1 to 26), —C₁-C₈ alkylene-(CH₂—O—CH₂)_{b}— (where b is 1 to 26), —C₁-C₈ alkylene-(CH₂—O—CH₂)_{b}—C₁-C₈ alkylene- (where b is 1 to 26), -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₁-C₈ alkylene-(CH₂—O—CH₂)_{b}—C(═O)— (where b is 1 to 26), —(CH₂—O—CH₂)_{b}—C₁-C₈alkylene-C(═O)— (where b is 1 to 26), —C₁-C₈ alkylene-(CH₂—O—CH₂)_{b}—C₁-C₈ alkylene-C(═O)— (where b is 1 to 26), —C₃-C₅ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₁-C₈ alkylene-(CH₂—O—CH₂)_{b}—NH— (where b is 1 to 26), —(CH₂—O—CH₂)_{b}—C₁-C₈ alkylene-NH— (where b is 1 to 26), —C₁-C₈ alkylene-(CH₂—O—CH₂)_{b}—C₁-C₈ alkylene-NH— (where b is 1 to 26), —C₁-C₈ alkylene-(C(═O))—NH—(CH₂—O—CH₂)_{b}—C(═O)— (where b is 1 to 26), —C₁-C₈ alkylene-(C(═O))—NH—(CH₂—O—CH₂)_{b}—C₁-C₈ alkylene-C(═O)— (where b is 1 to 26), —C₁-C₈ alkylene-NH—(C(═O))—(CH₂—O—CH₂)_{b}—NH— (where b is 1 to 26), —C₁-C₈ alkylene-NH—(C(═O))—(CH₂—O—CH₂)_{b}—C₁-C₈ alkylene-NH— (where b is 1 to 26), —C₃-C₈ carbocyclo-NH—, —O—(C₁-C₈ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, —C₁-C₁₀ heteroalkylene-S—, —C₃-C₈ carbocyclo-S—, —O—(C₁-C₈ alkyl)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—. Any of the R¹⁷ substituents can be substituted or unsubstituted (also referred to as non-substituted). In some aspects, the R¹⁷ substituents are unsubstituted. In some aspects, the R¹⁷ substituents are optionally substituted. In some aspects, the R¹⁷ groups (see., e.g., WO2013/173337) such as, for example, —(CH₂)ₓNH₂, —(CH₂)ₓNHR^{a}, and —(CH₂)ₓNR^{a}₂, wherein x is an integer of from 1-4 and each R^{a} is independently selected from the group consisting of C₁-C₆ alkyl and C₁-C₆ haloalkyl, or two R^{a} groups are combined with the nitrogen to which they are attached to form an azetidinyl, pyrrolidinyl or piperidinyl group.

In some embodiments of formula 100, R¹⁷ is —C₁-C₆ alkylene-C═O)—. In some embodiments, R¹⁷ is —C1 alkylene-C(═O)—.

In some embodiments of formula 100, R¹⁷ is —(CH₂—O—CH₂)_{b}—C₁-C₈ alkylene- (where b is 1 to 26), —C₁-C₈ alkylene-(CH₂—O—CH₂)_{b}— (where b is 1 to 26), —C₁-C₈ alkylene-(CH₂—O—CH₂)_{b}—C₁-C₈ alkylene-(where b is 1 to 26), —C₁-C₈ alkylene-(CH₂—O—CH₂)_{b}—C(═O)— (where b is 1 to 26), —(CH₂—O—CH₂)_{b}—C₁-C₈alkylene-C(═O)— (where b is 1 to 26), —C₁-C₈ alkylene-(CH₂—O—CH₂)_{b}—C₁-C₈ alkylene-C(═O)— (where b is 1 to 26), —C₁-C₈ alkylene-(CH₂—O—CH₂)_{b}—NH— (where b is 1 to 26), —(CH₂—O—CH₂)_{b}—C₁-C₈ alkylene-NH— (where b is 1 to 26), —C₁-C₈ alkylene-(CH₂—O—CH₂)_{b}—C₁-C₈ alkylene-NH— (where b is 1 to 26), —C₁-C₈ alkylene-(C(═O))—NH—(CH₂—O—CH₂)_{b}—C(═O)— (where b is 1 to 26), —C₁-C₈ alkylene-(C(═O))—NH—(CH₂—O—CH₂)_{b}—C₁-C₈ alkylene-C(═O)— (where b is 1 to 26), —C₁-C₈ alkylene-NH—(C(═O))—(CH₂—O—CH₂)_{b}—NH— (where b is 1 to 26), or —C₁-C₈ alkylene-NH—(C(═O))—(CH₂—O—CH₂)_{b}—C₁-C₈ alkylene-NH— (where b is 1 to 26).

In other embodiments, the Stretcher unit is linked to the Targeting unit via a disulfide bond between a sulfur atom of the Stretcher unit and a sulfur atom of the Targeting unit. A representative Stretcher unit of this embodiment is depicted in the following Formula 102, wherein L is the Targeting unit, the wavy line indicates an attachment site for an Amino Acid unit or a Linker Subunit L2 and R¹⁷ is as described above for Formulae 100 and 101.

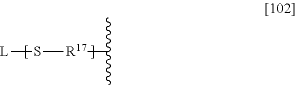

In yet another embodiment, a reactive group of a Stretcher unit contains a reactive site that can form a bond with a primary or secondary amino group of a Targeting unit. Examples of these reactive sites include, but are not limited to, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Representative Stretcher units of this embodiment are depicted in Formulas 103, 104, and 105, wherein L is a Targeting unit, the wavy line indicates an attachment site for an Amino Acid unit or a Linker Subunit L2 and R¹⁷ is as described above for Formula 100 and 101:

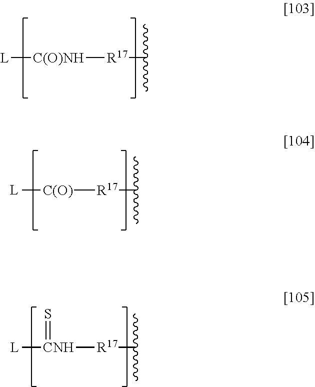

In yet another embodiment, a reactive group of a Stretcher unit contains a reactive site that is reactive to a modified carbohydrate's (—CHO) group that can be present on a Targeting unit. For example, a carbohydrate can be mildly oxidized using a reagent such as sodium periodate and the resulting (—CHO) unit of the oxidized carbohydrate can be condensed with a Stretcher unit that contains a functionality such as a hydrazide, an oxime, a primary or secondary amine, a hydrazine, a thiosemicarbazone, a hydrazine carboxyl, or an arylhydrazide (such as those described by Kaneko, T. et al. (1991) Bioconjugate Chem. 2:133-41.) Representative Stretcher units of this embodiment are depicted in the following Formulas 106, 107, and 108, wherein L is a Targeting unit, the wavy line indicates an attachment site for an Amino Acid unit or a Linker Subunit L2 and R¹⁷ is as described above for Formulae 100 and 101:

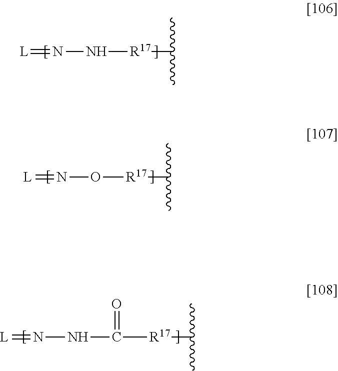

In some embodiments, it will be desirable to extend the length of a Stretcher unit. Accordingly, a Stretcher unit can comprise additional components. Representative Stretcher units of this embodiment are depicted in the following Formula 109, wherein L is a Targeting unit, the wavy line indicates an attachment site for an Amino Acid unit or a Linker Subunit L2 and R¹⁷ is as described above for Formula 100 and 101:

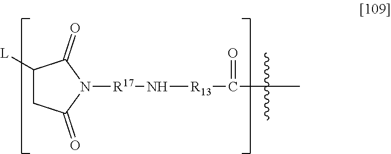

In some aspects of this embodiment, R¹⁷ is —C₁-C₅ alkylene-C(═O)—. R¹³ is —C₁-C₆ alkylene-, —(CH₂—O—CH₂)_{b}— (where b is 1 to 26), —C₃-C₈ carbocyclo-, -arylene-, —C₁-C₁₀ heteroalkylene-, —C₃-C₈ heterocyclo-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₁-C₁₀ alkylene-(C₃-C₈ heterocyclo)-, or —(C₃-C₈ heterocyclo)-C₁-C₁₀ alkylene-. In preferred embodiments, R¹³ is —(CH₂—O—CH₂)_{b}—, where b is 1 to 26.

### Targeting Units

In some embodiments, the Linkers are attached to Targeting units to form Targeting unit-Linkers. In some embodiments, the Linkers are attached to Targeting units via a Stretcher unit (L1) and to a Drug unit(s) via a Linker Subunit L2 to form a conjugate. In some embodiments, the Linkers are attached to a Targeting unit(s) via a Stretcher unit (L1) and to a Drug unit(s) via a Linker Subunit L2 for form a conjugate. In some embodiments, a Targeting unit is a protein, polypeptide or peptide. The Targeting units can be antibodies, antigen binding portions thereof or non-antibody targeting units. Non-antibody targeting units may also be referred to as non-antibody scaffolds.

In some embodiments, a Targeting unit specifically binds to a target molecule. As used herein, “specifically binds” refers to the ability of a Targeting unit (e.g., an antibody or portion thereof) described herein to bind to a target with a KD 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the Targeting unit and the concentration of target polypeptide. The person of ordinary skill in the art can determine appropriate conditions under which the antibodies, antibody binding portions and non-antibody scaffolds described herein selectively bind to a target using any suitable methods, such as titration of a binding agent in a suitable cell binding assay. A Targeting unit specifically bound to its target is not displaced by a non-similar competitor. In certain embodiments, a Targeting uni is said to specifically bind to its target when it preferentially recognizes its target in a complex mixture of proteins and/or macromolecules.

As used herein, the term “antibody” refers to an immunoglobulin molecule and immunologically active portions of immunoglobulin molecules, i.e., molecules that contain an antigen binding site(s) that specifically bind(s) to a target antigen. The term generally refers to antibodies comprised of two immunoglobulin heavy chain variable regions and two immunoglobulin light chain variable regions including full length antibodies (having heavy and light chain constant regions).

Each heavy chain is typically composed of a variable region (abbreviated as a VH region) and a constant region. The heavy chain constant region may include three domains CH1, CH2 and CH3 and optionally a fourth domain, CH4. Each light chain is composed of a variable region (abbreviated as a VL region) and a constant region. The light chain constant region is a CL domain. The VH and VL regions may be further divided into hypervariable regions referred to as complementarity-determining regions (CDRs) and interspersed with conserved regions referred to as framework regions (FR). Each VH and VL region thus includes three CDRs and four FRs that are arranged from the N terminus to the C terminus in the following order: FR1, CDR1, FR2, CDR2, FR3, CDR3, and FR4. This structure is well known to those skilled in the art.

As used herein, an “antigen-binding portion” of an antibody refers to the portions of an antibody having VH and/or VL sequences of an antibody or the CDRs of an antibody and that specifically binds to the target antigen. Examples of antigen binding portions include a Fab, a Fab′, a F(ab′)₂, a Fv, a scFv, a disulfide linked Fv, a single domain antibody (also referred to as a VHH, VNAR, sdAb, or nanobody) or a diabody (see, e.g., Huston et al., Proc. Natl. Acad. Sci. U.S.A., 85, 5879-5883 (1988) and Bird et al., Science 242, 423-426 (1988), which are incorporated herein by reference). As used herein, the terms Fab, F(ab′)₂ and Fv refer to the following: (i) a Fab is a monovalent fragment composed of the VL, VH, CL and CH1 domains; (ii) a F(ab′)₂ is a bivalent fragment comprising two Fab fragments linked to one another in the hinge region via a disulfide bridge; and (iii) a Fv composed of the VL and VH domains. Although the two domains of the Fv fragment, namely VL and VH, are encoded by separate coding regions, they may further be linked to one another using a synthetic linker, e.g., a poly-G4S amino acid sequence (‘(G₄S)ₙ’ disclosed as SEQ ID NO: 1, wherein n=1 to 5), making it possible to prepare them as a single protein chain in which the VL and VH regions combine in order to form monovalent molecules (known as single chain Fv or scFv). The term “antigen-binding portion” of an antibody is also intended to include such single chain antibodies. Other forms of single chain antibodies such as “diabodies” are likewise included here. Diabodies are bivalent, bispecific antibodies in which VH and VL regions are expressed on a single polypeptide chain, but using a linker connecting the VH and VL regions that is too short for the two regions to be able to combine on the same chain, thereby forcing the VH and VL regions to pair with complementary regions of a different chain (VL and VH, respectively), and to form two antigen-binding sites (see, for example, Holliger, R, et al. (1993) Proc. Natl. Acad. Sci. USA 90:64446448; Poljak, R. J, et al. (1994) Structure 2:1121-1123).

A single-domain antibody is an antigen binding portion of an antibody containing a single monomeric variable antibody region. Single domains antibodies can be derived from the variable region of the antibody heavy chain from camelids (e.g., nanobodies or VHH portions). Furthermore, the term single-domain antibody includes an autonomous human heavy chain variable domain (aVH) or VNAR portions derived from sharks (see, e.g., Hasler et al., Mol. Immunol. 75:28-37, 2016).

Techniques for producing single domain antibodies (e.g., DABs or VHH) are known in the art, as disclosed for example in Cossins et al. (2006, Prot Express Purif 51:253-259) and Li et al. (Immunol. Lett. 188:89-95, 2017). Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; and Maass et al., J Immunol Methods 324:13-25, 2007.) A VHH may have potent antigen-binding capacity and can interact with novel epitopes that are inaccessible to conventional VH-VL pairs (see, e.g., Muyldermans et al., 2001). Alpaca serum IgG contains about 50% camelid heavy chain only IgG antibodies (HCAbs) (see, e.g., Maass et al., 2007). Alpacas may be immunized with antigens and VHHs can be isolated that bind to and neutralize a target antigen (see, e.g., Maass et al., 2007). PCR primers that amplify alpaca VHH coding sequences have been identified and may be used to construct alpaca VHH phage display libraries, which can be used for antibody fragment isolation by standard biopanning techniques well known in the art (see, e.g., Maass et al., 2007).

In some embodiments, the Targeting unit is an antibody or antigen binding portion thereof is a bispecific or multispecific binding agent. Bispecific and multi-specific antibodies include the following: an scFv1-ScFv2, an ScFv1₂-Fc-scFv2₂, an IgG-scFv, a DVD-Ig, a triomab/quadroma, a two-in-one IgG, a scFv2-Fc, a TandAb, and an scFv-HSA-scFv. In some embodiments, an IgG-scFv is an IgG(H)-scFv, scFv-(H)IgG, IgG(L)-scFv, svFc-(L)IgG, 2scFV-IgG or IgG-2scFv. See, e.g., Brinkmann and Kontermann, MAbs 9(2):182-212 (2017); Wang et al., Antibodies, 2019, 8, 43; Dong et al., 2011, MAbs 3:273-88; Natsume et al., J. Biochem. 140(3):359-368, 2006; Cheal et al., Mol. Cancer Ther. 13(7):1803-1812, 2014; and Bates and Power, Antibodies, 2019, 8, 28.

In some embodiments, the Targeting unit binds to a target molecule, such as a cancer associated antigen such as CD19, CD20, CD30, CD33, CD38, CA125, MUC-1, prostate-specific membrane antigen (PSMA), CD44 surface adhesion molecule, mesothelin (MLSN), carcinoembryonic antigen (CEA), epidermal growth factor receptor (EGFR), EGFRvIII, vascular endothelial growth factor receptor-2 (VEGFR2), HER2, high molecular weight-melanoma associated antigen (HMW-MAA), MAGE-A1, IL-13R-a2, GD2, 1p19q, ABL1, AKT1, ALK, APC, AR, ATM, BRAF, BRCA1, BRCA2, cKIT, cMET, CSF1R, CTNNB1, FGFR1, FGFR2, FLT3, GNA11, GNAQ, GNAS, HRAS, IDH1, IDH2, JAK2, KDR (VEGFR2), KRAS, MGMT, MGMT-Me, MLH1, MPL, NOTCH1, NRAS, PDGFRA, Pgp, PIK3CA, PR, PTEN, RET, RRM1, SMO, SPARC, TLE3, TOP2A, TOPO1, TP53, TS, TUBB3, VHL, CDH1, ERBB4, FBXW7, HNF1A, JAK3, NPM1, PTPN11, RB1, SMAD4, SMARCB1, STK1, MLH1, MSH2, MSH6, PMS2, ROS1, ERCC1, 5T4 (TPBG), B7-H3, CCR7, CD105, CD22, CD46, CD47, CD56, CD70, CD71, CD79b, CDH6, CLDN6, CLDN18.2, CLEC12A, DLL3, DR5, ERBB3 (HER3), EPCAM, FOLR1, IGF1R, IL2RA (CD25), IL3RA, ITGB6, LIV-1, LRRC15, mesothelin (MSLN), NaPi2b (SLC34A2), nectin-4, PTK7, ROR1, SEZ6, SLC44A4, SLITRK6, Tissue Factor (TF), TROP2 or B7-H4. According to the invention, the terms “cancer associated antigen”, “tumor antigen”, “tumor expressed antigen”, “cancer antigen” “cancer associated antigen” and “cancer expressed antigen” are equivalents and are used interchangeably herein.

In some embodiments, a Targeting unit specifically binds to a target such as CD19, CD20, CD30, CD33, CD70, LIV-1, HER2, or EGFRv3.

In some embodiments, the Targeting unit is an antibody (or fragment thereof) that binds to a target having a sequences as disclosed in Leuschner et al., US 2022/0048951 and/or Lerchen et al., US 2022/0016258. Non-limiting examples of monoclonal antibodies include rituximab (Rituxan®), trastuzumab (Herceptin®), pertuzumab (Perjeta®)), bevacizumab (Avastin®), ranibizumab (Lucentis®), cetuximab (Erbitux®), alemtuzumab (Campath®), panitumumab (Vectibix®), ibritumomab (Zevalin®), tositumomab (Bexxar®), ipilimumab, zalutumumab, dalotuzumab, figitumumab, ramucirumab, galiximab, farletuzumab, ocrelizumab, ofatumumab (Arzerra®), the CD20 antibodies 2F2 (HuMax-CD20), 7D8, IgM2C6, IgG1 2C6, 11B8, B1, 2H7, LT20, iFS or AT80 (see Teeling et al., J. Immunol. 177:362-371 (2006)), daclizumab (Zenapax®), and anti-LHRH receptor antibodies such as clones A9E4, F1G4, AT2G7, GNRH03, GNRHR2, etc. which can be used in combination with, inter alia, a conjugate in accordance with the invention.

In some embodiments, a Targeting unit is a non-antibody scaffold. In some embodiments, a Targeting unit is a non-antibody protein scaffold. Such non-antibody scaffolds include, for example, Affibodies, Affilins, Anticalins, Atrimers, Avimers, Bicyclic peptides, Cys-knots, DARPins, FN3 scaffolds (e.g., Adnectins, Centyrins, Pronectins, and Tn3), Fynomers, Kunitz domains and OBodies. (See, e.g., Vazquez-Lombardi et al., Drug Discovery Today 20(10):1271 (2015) and the references cited therein.) Such Non-antibody protein scaffolds include, for example, Affibodies, Affilins, Anticalins, Atrimers, Avimers, Bicyclic peptides, Cys-knots, DARPins, FN3 scaffolds (e.g., Adnectins, Centyrins, Pronectins, and Tn3), Fynomers, Kunitz domains and OBodies. (See, e.g., Vazquez-Lombardi et al., Drug Discovery Today 20(10):1271 (2015) and the references cited therein.) Non-antibody scaffolds can be considered to fall into two structural categories, domain-sized constructs (in the range of 6 to 20 kDa), and constrained peptides (in the 2-4 kDa range). Domain-sized non-antibody scaffolds include, but are not limited to, affibodies, affilins, anticalins, atrimers, DARPins, FN3 scaffolds (such as adnectins and centyrins), fynomers, Kunitz domains, pronectins and OBodies. Peptide-sized non-antibody scaffolds include, for example, avimers, bicyclic peptides and cysteine knots. Non-antibody protein scaffolds can be considered to fall into two structural categories, domain-sized constructs (in the range of 6 to 20 kDa), and constrained peptides (in the 2-4 kDa range). Domain-sized non-antibody scaffolds include, but are not limited to, affibodies, affilins, anticalins, atrimers, DARPins, FN3 scaffolds (such as adnectins and centyrins), fynomers, Kunitz domains, pronectins and OBodies. Peptide-sized non-antibody scaffolds include, for example, avimers, bicyclic peptides and cysteine knots. These non-antibody scaffolds and the underlying proteins or peptides on which they are based or from which they have been derived are reviewed by, e.g., Simeon and Chen, Protein Cell 9(1): 3-14 (2018); Vazquez-Lombardi et al., Drug Discovery Today 20: 1271-1283 (2015), and by Binz et al., Nature Biotechnol. 23: 1257-1268 (2005), the contents of each of which are herein incorporated by reference in their entireties.

Advantages of using non-antibody scaffolds include increased affinity, target neutralization, and stability. Various non-antibody scaffolds also can overcome some of the limitations of antibody scaffolds, e.g., in terms of tissue penetration, smaller size, and thermostability. Some non-antibody scaffolds can also permit easier construction, not being hindered, for example, by potential light chain association concerns when bispecific constructs are desired. Methods of constructing constructs on a non-antibody scaffold are known to those of ordinary skill in the art.

Accordingly, in some embodiments, a Targeting unit can comprise a non-antibody scaffold. Accordingly, in some embodiments, a Targeting unit can comprise a non-antibody scaffold protein. One of skill in the art would appreciate that a Targeting unit can include, in some embodiments, e.g., an adnectin scaffold or a portion derived from human tenth fibronectin type III domain (10fn3); an anticalin scaffold derived from human lipocalin (e.g., such as those described in, e.g., WO2015/104406); an avimer scaffold or a protein fragment derived from the A-domain of low density-related protein (LRP) and/or very low density lipoprotein receptor (VLDLR); a fynomer scaffold or portion of the SH3 domain of FYN tyrosine kinase; a kunitz domain scaffold or portion of Kunitz-type protease inhibitors, such as a human trypsin inhibitor, aprotinin (bovine pancreatic trypsin inhibitor), Alzheimer's amyloid precursor protein, and tissue factor pathway inhibitor; a knottin scaffold (cysteine knot miniproteins), such as one based on a trypsin inhibitor from E. elaterium; an affibody scaffold or all or part of the Z domain of S. aureus protein A; a β-Hairpin mimetic scaffold; a Designed ankyrin repeat protein (DARPin) scaffold or artificial protein scaffolds based on ankyrin repeat (AR) proteins; or any scaffold derived or based on human transferrin, human CTLA-4, human crystallin, and human ubiquitin. For example, the binding site of human transferrin for human transferrin receptor can be diversified to create a diverse library of transferrin variants, some of which have acquired affinity for different antigens. See, e.g., Ali et al. (1999) J. Biol. Chem. 274:24066-24073. The portion of human transferrin not involved with binding the receptor remains unchanged and serves as a scaffold, like framework regions of antibodies, to present the variant binding sites. The libraries are then screened, as an antibody library is, and in accordance with the methods described herein, against a target antigen of interest to identify those variants having optimal selectivity and affinity for the target antigen. See, e.g., Hey et al. (2005) TRENDS Biotechnol. 23(10):514-522.

### Constant Regions

In some embodiments, a Targeting unit, such as an antibody or antigen-binding portion thereof or other Targeting unit, has an antibody constant region(s). In some embodiments, the constant region is a fully human constant region(s). In some embodiments, the constant region is a humanized constant region(s). In some embodiments, the constant region is a non-human constant region(s). An immunoglobulin constant region refers to a heavy or light chain constant region. Human heavy chain and light chain constant region amino acid sequences are known in the art. A constant region can be of any suitable type, which can be selected from the classes of immunoglobulins, IgA, IgD, IgE, IgG, and IgM. Several immunoglobulin classes can be further divided into isotypes, e.g., IgG1, IgG2, IgG3, IgG4, or IgAQ1, and IgA2. The heavy-chain constant regions (Fc) that correspond to the different classes of immunoglobulins can be α, δ, ε, γ, and μ, respectively. The light chains can be one of either kappa (or κ) and lambda (or λ).

In some embodiments, a constant region can have an IgG isotype. In some embodiments, a constant region can have an IgG1 isotype. In some embodiments, a constant region can have an IgG2 isotype. In some embodiments, a constant region can have an IgG3 isotype. In some embodiments, a constant region can have an IgG4 isotype. In some embodiments, a constant region can have a hybrid isotype comprising constant regions from two or more isotypes. In some embodiments, an immunoglobulin constant region can be an IgG1 or IgG4 constant region. In some embodiments, a constant region is of the IgG1 isotype and has the amino acid sequence set forth in SEQ ID NO:2. In some embodiments, a constant region is of the kappa isotype and has the amino acid sequence set forth in SEQ ID NO:3.

Furthermore, a Targeting unit comprising an antibody or an antigen-binding portion thereof or non-antibody scaffold may be part of a larger molecule formed by covalent or noncovalent association of the antibody or antigen binding portion with one or more other proteins or peptides. Relevant to such Targeting units are the use, for example, of the streptavidin core region in order to prepare a tetrameric scFv molecule (Kipriyanov, S. M., et al. (1995), Human Antibodies and Hybridomas 6:93-101) and the use of a cysteine residue, a marker peptide and a C-terminal polyhistidinyl peptide, e.g. hexahistidinyl tag (‘hexahistidinyl tag’ disclosed as SEQ ID NO: 4) in order to produce bivalent and biotinylated scFv molecules (Kipriyanov, S. M., et al. (1994) Mol. Immunol. 31:10471058).

### Fc Domain Modifications to Alter Effector Function

In some embodiments, an Fc region or Fc domain of a Targeting unit, such as an antibody or antigen binding portion thereof or non-antibody scaffold, has substantially no binding to at least one Fc receptor selected from FcγRI (CD64), FcγRIIA (CD32a), FcγRIIB (CD32b), FcγRIIIA (CD16a), and FcγRIIIB (CD16b). In some embodiments, an Fc region or domain exhibits substantially no binding to any of the Fc receptors selected from FcγRI (CD64), FcγRIIA (CD32a), FcγRIIB (CD32b), FcγRIIIA (CD16a), and FcγRIIIB (CD16b). As used herein, “substantially no binding” refers to weak to no binding to a selected Fcgamma receptor or receptors. In some embodiments, “substantially no binding” refers to a reduction in binding affinity (i.e., increase in Kd) to a Fc gamma receptor of at least 1000-fold. In some embodiments, an Fc domain or region is an Fc null. As used herein, an “Fc null” refers to an Fc region or Fc domain that exhibits weak to no binding to any of the Fcgamma receptors. In some embodiments, an Fc null domain or region exhibits a reduction in binding affinity (i.e., increase in Kd) to Fc gamma receptors of at least 1000-fold.

In some embodiments, an Fc domain has reduced or substantially no effector function activity. As used herein, “effector function activity” refers to antibody dependent cellular cytotoxicity (ADCC), antibody dependent cellular phagocytosis (ADCP) and/or complement dependent cytotoxicity (CDC). In some embodiments, an Fc domain exhibits reduced ADCC, ADCP or CDC activity, as compared to a wildtype Fc domain. In some embodiments, an Fc domain exhibits a reduction in ADCC, ADCP and CDC, as compared to a wildtype Fc domain. In some embodiments, an Fc domain exhibits substantially no effector function (i.e., the ability to stimulate or effect ADCC, ADCP or CDC). As used herein, “substantially no effector function” refers to a reduction in effector function activity of at least 1000-fold, as compared to a wildtype or reference Fc domain.

In some embodiments, an Fc domain has reduced or no ADCC activity. As used herein reduced or no ADCC activity refers to a decrease in ADCC activity of an Fc domain by a factor of at least 10, at least 20, at least 30, at least 50, at least 100 or at least 500.

In some embodiments, an Fc domain has reduced or no CDC activity. As used herein reduced or no CDC activity refers to a decrease in CDC activity of an Fc domain by a factor of at least 10, at least 20, at least 30, at least 50, at least 100 or at least 500.

In vitro and/or in vivo cytotoxicity assays can be conducted to confirm the reduction/depletion of ADCC and/or CDC activity. For example, Fc receptor (FcR) binding assays can be conducted to ensure that the antibody lacks Fcgamma receptor binding (hence likely lacking ADCC activity). The primary cells for mediating ADCC, NK cells, express FcgammaRIII only, whereas monocytes express FcgammaRI, FcgammaRII and FcgammaRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest are described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat'l Acad. Sci. USA 95:652-656 (1998).

C1q binding assays may also be carried out to confirm that an antibody or Fc domain or region is unable to bind C1q and hence lacks CDC activity or has reduced CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)).

In some embodiments, an Fc domain has reduced or no ADCP activity. As used herein reduced or no ADCP activity refers to a decrease in ADCP activity of an Fc domain by a factor of at least 10, at least 20, at least 30, at least 50, at least 100 or at least 500.

ADCP binding assays may also be carried out to confirm that an antibody or Fc domain or region lacks ADCP activity or has reduced ADCP activity. See, e.g., US20190079077 and US20190048078 and the references disclosed therein.

A Targeting unit, such as an antibody or antigen binding portion thereof or non-antibody scaffold, with reduced effector function activity includes those with substitution of one or more of Fc region residues, such as, for example, 238, 265, 269, 270, 297, 327 and 329, according to the EU number of Kabat (see, e.g., U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine, according to the EU numbering of Kabat (see U.S. Pat. No. 7,332,581). Certain antibody variants with diminished binding to FcRs are also known. (See, e.g., U.S. Pat. No. 6,737,056; WO 2004/056312, and Shields et al., J. Biol. Chem. 9(2): 6591-6604 (2001).) A Targeting unit, such as an antibody or antigen binding portion thereof or non-antibody scaffold, with diminished binding to FcRs can be prepared containing such amino acid modifications.

In some embodiments, a Targeting unit, such as an antibody or antigen binding portion thereof or non-antibody scaffold, comprises an Fc domain or region with one or more amino acid substitutions which diminish FcgammaR binding, e.g., substitutions at positions 234 and 235 of the Fc region (EU numbering of residues). In some embodiments, the substitutions are L234A and L235A (LALA), according to the EU numbering of Kabat. In some embodiments, the Fc domain comprises D265A and/or P329G in an Fc region derived from a human IgG1 Fc region, according to the EU numbering of Kabat. In some embodiments, the substitutions are L234A, L235A and P329G (LALA-PG), according to the EU numbering of Kabat, in an Fc region derived from a human IgG1 Fc region. (See, e.g., WO 2012/130831). In some embodiments, the substitutions are L234A, L235A and D265A (LALA-DA) in an Fc region derived from a human IgG1 Fc region, according to the EU numbering of Kabat.

In some embodiments, alterations are made in the Fc region that result in altered (i.e., either diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

### Methods of Making Antibodies and Antigen Binding Portions and Other Targeting Units

In various embodiments, Targeting units such as antibodies and antigen binding portions thereof, can be produced in human, murine or other animal-derived cells lines. Recombinant DNA expression can be used to produce antibodies and antigen binding portions thereof. This allows the production of antibodies as well as a spectrum of antigen binding portions (including fusion proteins) in a host species of choice. The production of antibodies and antigen binding portions thereof in bacteria, yeast, transgenic animals and chicken eggs are also alternatives for cell-based production systems. The main advantages of transgenic animals are potential high yields from renewable sources.

Nucleic acid molecules encoding the amino acid sequence(s) of Targeting unit, such as an antibody or antigen binding portion thereof can be prepared by a variety of methods known in the art. These methods include, but are not limited to, preparation of synthetic nucleotide sequences encoding of an antibody or antigen binding portion. In addition, oligonucleotide-mediated (or site-directed) mutagenesis, PCR-mediated mutagenesis, and cassette mutagenesis can be used to prepare nucleotide sequences encoding an antibody or antigen binding portion. A nucleic acid sequence encoding at least an antibody or antigen binding portion thereof, or a polypeptide thereof, as described herein, can be recombined with vector DNA in accordance with conventional techniques, such as, for example, blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases or other techniques known in the art. Techniques for such manipulations are disclosed, e.g., by Maniatis et al., Molecular Cloning, Lab. Manual (Cold Spring Harbor Lab. Press, NY, 1982 and 1989), and Ausubel et al., Current Protocols in Molecular Biology (John Wiley & Sons), 1987-1993, and can be used to construct nucleic acid sequences and vectors that encode an antibody or antigen binding portion thereof or a VH or VL polypeptide thereof.

As used herein, the terms “nucleic acid” or “nucleic acid sequence” or “polynucleotide sequence” or “nucleotide” refers to a polymeric molecule incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. In some embodiments, the nucleic acid can be a cDNA, e.g., a nucleic acid lacking introns.

A nucleic acid molecule, such as DNA, is said to be “capable of expressing” a polypeptide if it contains nucleotide sequences that contain transcriptional and translational regulatory information and such sequences are “operably linked” to nucleotide sequences that encode the polypeptide. An operable linkage is a linkage in which the regulatory DNA sequences and the DNA sequence sought to be expressed (e.g., an antibody or antigen binding portion thereof) are connected in such a way as to permit gene expression of a polypeptide(s) or antigen binding portions in recoverable amounts. The precise nature of the regulatory regions needed for gene expression may vary from organism to organism, as is well known in the analogous art. See, e.g., Sambrook et al., 1989; Ausubel et al., 1987-1993.

Accordingly, the expression of a Targeting unit, such as an antibody or antigen-binding portion thereof, can occur in either prokaryotic or eukaryotic cells. Suitable hosts include bacterial or eukaryotic hosts, including yeast, insects, fungi, bird and mammalian cells either in vivo or in situ, or host cells of mammalian, insect, bird or yeast origin. The mammalian cell or tissue can be of human, primate, hamster, rabbit, rodent, cow, pig, sheep, horse, goat, dog or cat origin, but other mammalian cells may be used. Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of an antibody or antigen binding portion thereof as described herein with a specified amino terminus sequence. Moreover, problems associated with retention of initiation codon-derived methionine residues in direct yeast (or bacterial) expression maybe avoided. (See, e.g., Sabin et al., 7 Bio/Technol. 705 (1989); Miller et al., 7 Bio/Technol. 698 (1989).) Any of a series of yeast gene expression systems incorporating promoter and termination elements from the actively expressed genes coding for glycolytic enzymes produced in large quantities when yeast are grown in medium rich in glucose can be utilized to obtain recombinant antibodies or antigen-binding portions thereof. Known glycolytic genes can also provide very efficient transcriptional control signals. For example, the promoter and terminator signals of the phosphoglycerate kinase gene can be utilized.

Production of antibodies or antigen-binding portions in insects can be achieved, for example, by infecting an insect host with a baculovirus engineered to express a polypeptide by methods known to those of ordinary skill in the art. See Ausubel et al., 1987-1993.

In some embodiments, the introduced nucleic acid sequence(s) (encoding an antibody or antigen binding portion thereof or a polypeptide thereof) is incorporated into a plasmid or viral vector capable of autonomous replication in a recipient host cell. Any of a wide variety of vectors can be employed for this purpose and are known and available to those of ordinary skill in the art. See, e.g., Ausubel et al., 1987-1993. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Exemplary prokaryotic vectors known in the art include plasmids such as those capable of replication in E. coli. Other gene expression elements useful for the expression of DNA encoding antibodies or antigen-binding portions thereof include, but are not limited to (a) viral transcription promoters and their enhancer elements, such as the SV40 early promoter. (Okayama et al., 3 Mol. Cell. Biol. 280 (1983)), Rous sarcoma virus LTR (Gorman et al., 79 PNAS 6777 (1982)), and Moloney murine leukemia virus LTR (Grosschedl et al., 41 Cell 885 (1985)); (b) splice regions and polyadenylation sites such as those derived from the SV40 late region (Okayarea et al., 1983), and (c) polyadenylation sites such as in SV40 (Okayama et al., 1983). Immunoglobulin-encoding DNA genes can be expressed as described by Liu et al., infra, and Weidle et al., 51 Gene 21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin encoding nucleotide sequences, the transcriptional promoter can be, for example, human cytomegalovirus, the promoter enhancers can be cytomegalovirus and mouse/human immunoglobulin.

In some embodiments, for expression of DNA coding regions in rodent cells, the transcriptional promoter can be a viral LTR sequence, the transcriptional promoter enhancers can be either or both the mouse immunoglobulin heavy chain enhancer and the viral LTR enhancer, and the polyadenylation and transcription termination regions. In other embodiments, DNA sequences encoding other proteins are combined with the above-recited expression elements to achieve expression of the proteins in mammalian cells.

Each coding region or gene fusion is assembled in, or inserted into, an expression vector. Recipient cells capable of expressing the variable region(s) or antigen binding portions thereof are then transfected singly with nucleotides encoding an antibody or an antibody polypeptide or antigen-binding portion thereof, or are co-transfected with a polynucleotide(s) encoding VH and VL chain coding regions. The transfected recipient cells are cultured under conditions that permit expression of the incorporated coding regions and the expressed antibody chains or intact antibodies or antigen binding portions are recovered from the culture.

In some embodiments, the nucleic acids containing the coding regions encoding an antibody or antigen-binding portion thereof are assembled in separate expression vectors that are then used to co-transfect a recipient host cell. Each vector can contain one or more selectable genes. For example, in some embodiments, two selectable genes are used, a first selectable gene designed for selection in a bacterial system and a second selectable gene designed for selection in a eukaryotic system, wherein each vector has a set of coding regions. This strategy results in vectors which first direct the production, and permit amplification, of the nucleotide sequences in a bacterial system. The DNA vectors so produced and amplified in a bacterial host are subsequently used to co-transfect a eukaryotic cell, and allow selection of a co-transfected cell carrying the desired transfected nucleic acids (e.g., containing antibody heavy and light chains). Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively the fused nucleotide sequences encoding VH and VL chains can be assembled on the same expression vector.

For transfection of the expression vectors and production of antibodies or antigen binding portions thereof, the recipient cell line can be a Chinese Hamster ovary cell line (e.g., DG44) or a myeloma cell. Myeloma cells can synthesize, assemble and secrete immunoglobulins encoded by transfected immunoglobulin genes and possess the mechanism for glycosylation of the immunoglobulin. For example, in some embodiments, the recipient cell is the recombinant Ig-producing myeloma cell SP2/0. SP2/0 cells only produce immunoglobulins encoded by the transfected genes. Myeloma cells can be grown in culture or in the peritoneal cavity of a mouse, where secreted immunoglobulin can be obtained from ascites fluid.

An expression vector encoding an antibody or antigen-binding portion thereof can be introduced into an appropriate host cell by any of a variety of suitable means, including such biochemical means as transformation, transfection, protoplast fusion, calcium phosphate-precipitation, and application with polycations such as diethylaminoethyl (DEAE) dextran, and such mechanical means as electroporation, direct microinjection and microprojectile bombardment, as known to one of ordinary skill in the art. (See, e.g., Johnston et al., 240 Science 1538 (1988)).

Yeast provides certain advantages over bacteria for the production of immunoglobulin heavy and light chains. Yeasts carry out post-translational peptide modifications including glycosylation. A number of recombinant DNA strategies exist that utilize strong promoter sequences and high copy number plasmids which can be used for production of the desired proteins in yeast. Yeast recognizes leader sequences of cloned mammalian gene products and secretes polypeptides bearing leader sequences (i.e., pre-polypeptides). See, e.g., Hitzman et al., 11th Intl. Conf Yeast, Genetics & Molec. Biol. (Montpelier, France, 1982).

Yeast gene expression systems can be routinely evaluated for the levels of production, secretion and the stability of antibodies, and assembled antibodies and antigen binding portions thereof. Various yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. Another example is the translational elongation factor 1alpha promoter, such as that from Chinese hamster cells. A number of approaches can be taken for evaluating optimal expression plasmids for the expression of immunoglobulins in yeast. See II DNA Cloning 45, (Glover, ed., IRL Press, 1985) and e.g., U.S. Publication No. US 2006/0270045 A1.

Bacterial strains can also be utilized as hosts for the production of the antibody molecules or antigen binding portions thereof as described herein. E. coli K12 strains such as E. coli W3110, Bacillus species, enterobacteria such as Salmonella typhimurium or Serratia marcescens, and various Pseudomonas species can be used. Plasmid vectors containing replicon and control sequences that are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of antibodies and antigen binding portions thereof in bacteria (see Glover, 1985; Ausubel, 1987, 1993; Sambrook, 1989; Colligan, 1992-1996).

Host mammalian cells can be grown in vitro or in vivo. Mammalian cells provide post-translational modifications to immunoglobulin molecules including leader peptide removal, folding and assembly of VH and VL chains, glycosylation of the antibody molecules, and secretion of functional antibody and/or antigen binding portions thereof.

Mammalian cells which can be useful as hosts for the production of antibody proteins, in addition to the cells of lymphoid origin described above, include cells of fibroblast origin, such as Vero or CHO-K1 cells. Exemplary eukaryotic cells that can be used to express immunoglobulin polypeptides include, but are not limited to, COS cells, including COS 7 cells; 293 cells, including 293-6E cells; CHO cells, including CHO—S and DG44 cells; PERC6™ cells (Crucell); and NSO cells. In some embodiments, a particular eukaryotic host cell is selected based on its ability to make desired post-translational modifications to the heavy chains and/or light chains. For example, in some embodiments, CHO cells produce polypeptides that have a higher level of sialylation than the same polypeptide produced in 293 cells.

In some embodiments, one or more antibodies or antigen-binding portions thereof can be produced in vivo in an animal that has been engineered or transfected with one or more nucleic acid molecules encoding the polypeptides, according to any suitable method.

In some embodiments, an antibody or antigen-binding portion thereof is produced in a cell-free system. Non-limiting exemplary cell-free systems are described, e.g., in Sitaraman et al., Methods Mol. Biol. 498: 229-44 (2009); Spirin, Trends Biotechnol. 22: 538-45 (2004); and Endo et al., Biotechnol. Adv. 21: 695-713 (2003).

Many vector systems are available for the expression of the VH and VL chains in mammalian cells (see Glover, 1985). Various approaches can be followed to obtain intact antibodies. As discussed above, it is possible to co-express VH and VL chains and optionally the associated constant regions in the same cells to achieve intracellular association and linkage of VH and VL chains into complete tetrameric H₂L₂ antibodies or antigen-binding portions thereof. The co-expression can occur by using either the same or different plasmids in the same host. Nucleic acids encoding the VH and VL chains or antigen binding portions thereof can be placed into the same plasmid, which is then transfected into cells, thereby selecting directly for cells that express both chains. Alternatively, cells can be transfected first with a plasmid encoding one chain, for example the VL chain, followed by transfection of the resulting cell line with a VH chain plasmid containing a second selectable marker. Cell lines producing antibodies or antigen-binding portions thereof via either route could be transfected with plasmids encoding additional copies of peptides, VH, VL, or VH plus VL chains in conjunction with additional selectable markers to generate cell lines with enhanced properties, such as higher production of assembled antibodies or antigen binding portions thereof or enhanced stability of the transfected cell lines.

Additionally, plants have emerged as a convenient, safe and economical alternative expression system for recombinant antibody production, which are based on large scale culture of microbes or animal cells. Antibodies or antigen binding portions thereof can be expressed in plant cell culture, or plants grown conventionally. The expression in plants may be systemic, limited to sub-cellular plastids, or limited to seeds (endosperms). See, e.g., U.S. Patent Pub. No. 2003/0167531; U.S. Pat. Nos. 6,080,560; 6,512,162; and WO 0129242. Several plant-derived antibodies have reached advanced stages of development, including clinical trials (see, e.g., Biolex, N.C.).

For intact antibodies, the variable regions (VH and VL regions) of antibodies are typically linked to at least a portion of an immunoglobulin constant region (Fc) or domain, typically that of a human immunoglobulin. Human constant region DNA sequences can be isolated in accordance with well-known procedures from a variety of human cells, such as immortalized B-cells (WO 87/02671). An antibody can contain both light chain and heavy chain constant regions. The heavy chain constant region can include CH1, hinge, CH2, CH3, and, optionally, CH4 regions. In some embodiments, the CH2 domain can be deleted or omitted.

Techniques described for the production of single chain antibodies (see, e.g. U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989); which are incorporated by reference herein in their entireties) can be adapted to produce single chain antibodies that specifically bind to the target antigen. Single chain antibodies are formed by linking the heavy and light chain variable regions of the Fv region via an amino acid bridge, resulting in a single chain polypeptide. Techniques for the assembly of functional Fv portions in E. coli can also be used (see, e.g. Skerra et al., Science 242:1038-1041 (1988); which is incorporated by reference herein in its entirety).

In some embodiments, an antigen binding portion comprises one or more scFvs. An scFv can be, for example, a fusion protein of the variable regions of the heavy (VH) and light chain (VL) variable regions of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96. Methods for making scFv molecules and designing suitable peptide linkers are described in, for example, U.S. Pat. Nos. 4,704,692; 4,946,778; Raag and Whitlow, FASEB 9:73-80 (1995) and Bird and Walker, TIBTECH, 9: 132-137 (1991). scFv-Fcs have been described by Sokolowska-Wedzina et al., Mol. Cancer Res. 15(8):1040-1050, 2017.

In some embodiments, an antigen binding portion is a single-domain antibody is an antibody portion consisting of a single monomeric variable antibody domain. Single domains antibodies can be derived from the variable domain of the antibody heavy chain from camelids (e.g., nanobodies or VHH portions). Furthermore, a single-domain antibody can be an autonomous human heavy chain variable domain (aVH) or VNAR portions derived from sharks (see, e.g., Hasler et al., Mol. Immunol. 75:28-37, 2016).

Techniques for producing single domain antibodies (DABs or VHH) are known in the art, as disclosed for example in Cossins et al. (2006, Prot Express Purif 51:253-259) and Li et al. (Immunol. Lett. 188:89-95, 2017). Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; and Maass et al., J Immunol Methods 324:13-25, 2007.) A VHH may have potent antigen-binding capacity and can interact with epitopes that are inaccessible to conventional VH-VL pairs (see, e.g., Muyldermans et al., 2001). Alpaca serum IgG contains about 50% camelid heavy chain only IgG antibodies (HCAbs) (see, e.g., Maass et al., 2007). Alpacas may be immunized with antigens and VHHs can be isolated that bind to and neutralize the target antigen (see, e.g., Maass et al., 2007). PCR primers that amplify alpaca VHH coding sequences have been identified and can be used to construct alpaca VHH phage display libraries, which can be used for antibody fragment isolation by standard biopanning techniques well known in the art (see, e.g., Maass et al., 2007).

Techniques for making multispecific antibodies include, but are not limited to, recombinant co-expression of two immunoglobulin heavy chain-light chain pairs having different specificities (see, e.g., Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168; Carter (2001), J Immunol Methods 248, 7-15). Multi-specific antibodies may also be made by engineering electrostatic steering effects for making antibody Fc-heterodimeric molecules (see, e.g., WO 2009/089004A1); cross-linking of two or more antibodies or antigen binding portions thereof (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody portions (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” also can be Targeting units (see, e.g. US 2006/0025576A1).

In some embodiments, the Targeting units comprise different antigen-binding sites, fused to one or the other of the two subunits of the Fc domain; thus, the two subunits of the Fc domain may be comprised in two non-identical polypeptide chains. Recombinant co-expression of these polypeptides and subsequent dimerization leads to several possible combinations of the two polypeptides. To improve the yield and purity of the bispecific molecules in recombinant production, it will thus be advantageous to introduce in the Fc domain of the Targeting unit a modification promoting the association of the desired polypeptides.

Generally, this method involves replacement of one or more amino acid residues at the interface of the two Fc domains by charged amino acid residues so that homodimer formation becomes electrostatically unfavorable but heterodimerization electrostatically favorable.

In some embodiments, a Targeting unit is a “bispecific T cell engager” or BiTE (see, e.g., WO2004/106381, WO2005/061547, WO2007/042261, and WO2008/119567). This approach utilizes two antibody variable domains arranged on a single polypeptide. For example, a single polypeptide chain can include two single chain Fv (scFv) portions, each having a variable heavy chain (VH) and a variable light chain (VL) domain separated by a polypeptide linker of a length sufficient to allow intramolecular association between the two domains. This single polypeptide further includes a polypeptide spacer sequence between the two scFvs. Each scFv recognizes a different epitope, and these epitopes may be specific for different proteins, such that both proteins are bound by the BiTE.

As it is a single polypeptide, the bispecific T cell engager may be expressed using any prokaryotic or eukaryotic cell expression system known in the art, e.g., a CHO cell line. However, specific purification techniques (see, e.g., EP1691833) may be necessary to separate monomeric bispecific T cell engagers from other multimeric species, which may have biological activities other than the intended activity of the monomer. In one exemplary purification scheme, a solution containing secreted polypeptides is first subjected to a metal affinity chromatography, and polypeptides are eluted with a gradient of imidazole concentrations. This eluate is further purified using anion exchange chromatography, and polypeptides are eluted using with a gradient of sodium chloride concentrations. Finally, this eluate is subjected to size exclusion chromatography to separate monomers from multimeric species. In some embodiments, a Targeting unit is a bispecific antibody is composed of a single polypeptide chain comprising two single chain FV portions (scFV) fused to each other by a peptide linker.

In some embodiments, a Targeting unit is multispecific, such as an IgG-scFV. IgG-scFv formats include IgG(H)-scFv, scFv-(H)IgG, IgG(L)-scFv, svFc-(L)IgG, 2scFV-IgG and IgG-2scFv. These and other bispecific antibody formats and methods of making them have been described in for example, Brinkmann and Kontermann, MAbs 9(2):182-212 (2017); Wang et al., Antibodies, 2019, 8, 43; Dong et al., 2011, MAbs 3:273-88; Natsume et al., J. Biochem. 140(3):359-368, 2006; Cheal et al., Mol. Cancer Ther. 13(7):1803-1812, 2014; and Bates and Power, Antibodies, 2019, 8, 28.

Igg-like dual-variable domain antibodies (DVD-Ig) have been described by Wu et al., 2007, Nat Biotechnol 25:1290-97; Hasler et al., Mol. Immunol. 75:28-37, 2016 and in WO 08/024188 and WO 07/024715. Triomabs have been described by Chelius et al., MAbs 2(3):309-319, 2010. 2-in-1-IgGs have been described by Kontermann et al., Drug Discovery Today 20(7):838-847, 2015. Tanden antibody or TandAb have been described by Kontermann et al., id. ScFv-HSA-scFv antibodies have also been described by Kontermann et al. (id.).

Intact (e.g., whole) antibodies, their dimers, individual light and heavy chains, or antigen binding portions thereof can be recovered and purified by known techniques, e.g., immunoadsorption or immunoaffinity chromatography, chromatographic methods such as HPLC (high performance liquid chromatography), ammonium sulfate precipitation, gel electrophoresis, or any combination of these. See generally, Scopes, Protein Purification (Springer-Verlag, N.Y., 1982). Substantially pure antibodies or antigen binding portions thereof of at least about 90% to 95% homogeneity are advantageous, as are those with 98% to 99% or more homogeneity, particularly for pharmaceutical uses. Once purified, partially or to homogeneity as desired, an intact antibody or antigen binding portions thereof can then be used therapeutically or in developing and performing assay procedures, immunofluorescent staining, and the like. See generally, Vols. I & II Immunol. Meth. (Lefkovits & Pernis, eds., Acad. Press, NY, 1979 and 1981).

### Drug Units

In some embodiments, the Linkers are attached to a Drug unit(s), a Targeting unit and/or to a Targeting unit and to a Drug unit(s) (the latter also referred to as a conjugate, ADC or antibody drug conjugate). In some embodiments, a Linker via a Linker Subunit L2, is attached to at least one Drug unit. As used herein, in the context of a conjugate, the term “Drug unit” or drug refers to cytotoxic agents (such as chemotherapeutic agents or drugs), immunomodulatory agents, nucleic acids (including siRNAs), growth inhibitory agents, toxins (e.g., protein toxins, enzymatically active toxins of bacterial, fungal, plant, or animal origin, or fragments thereof), radioactive isotopes, PROTACs and other compounds that are active against target cells when delivered to those cells.

### Cytotoxic Agents

In some embodiments, a Drug unit is a cytotoxic agent. A “cytotoxic agent” refers to an agent that has a cytotoxic effect on a cell. A “cytotoxic effect” refers to the depletion, elimination and/or the killing of a target cell(s). Cytotoxic agents include, for example, tubulin disrupting agents, topoisomerase inhibitors, DNA minor groove binders, and DNA alkylating agents.

Tubulin disrupting agents include, for example, auristatins, dolastatins, tubulysins, colchicines, vinca alkaloids, taxanes, cryptophycins, maytansinoids, hemiasterlins, as well as other tubulin disrupting agents. Auristatins are derivatives of the natural product dolastatin 10. Exemplary auristatins include MMAE (N-methylvaline-valine-dolaisoleuine-dolaproine-norephedrine), MMAF (N-methylvaline-valine-dolaisoleuine-dolaproine-phenylalanine) and AFP (see WO2004/010957 and WO2007/008603). Other auristatin like compounds are disclosed in, for example, Published US Application Nos. US2021/0008099, US2017/0121282, US2013/0309192 and US2013/0157960. Dolastatins include, for example, dolastatin 10 and dolastatin 15 (see, e.g., Pettit et al., J. Am. Chem. Soc., 1987, 109, 6883-6885; Pettit et al., Anti-Cancer Drug Des., 1998, 13, 243-277; and Published US Application US2001/0018422). Additional dolastatin derivatives contemplated for use herein are disclosed in U.S. Pat. No. 9,345,785, incorporated herein by reference.

Tubulysins include, but are not limited to, tubulysin D, tubulysin M, tubuphenylalanine and tubutyrosine. WO2017/096311 and WO/2016-040684 describe tubulysin analogs including tubulysin M.

Colchicines include, but are not limited to, colchicine and CA-4.

Vinca alkaloids include, but are not limited to, vinblastine (VBL), vinorelbine (VRL), vincristine (VCR) and vindesine (VOS).

Taxanes include, but are not limited to, paclitaxel and docetaxel.

Cryptophycins include but are not limited to cryptophycin-1 and cryptophycin-52.

Maytansinoids include, but are not limited to, maytansine, maytansinol, maytansine analogs in DM1, DM3 and DM4, and ansamatocin-2. Exemplary maytansinoid drug moieties include those having a modified aromatic ring, such as: C-19-dechloro (U.S. Pat. No. 4,256,746) (prepared by lithium aluminum hydride reduction of ansamitocin P2); C-20-hydroxy (or C-20-demethyl) +/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides), and those having modifications at other positions.

Maytansinoid drug moieties also include those having modifications such as: C-9-SH (U.S. Pat. No. 4,424,219) (prepared by the reaction of maytansinol with H₂S or P₂S₅); C-14-alkoxymethyl(demethoxy/CH₂OR) (see, U.S. Pat. No. 4,331,598); C-14-hydroxymethyl or acyloxymethyl (CH₂OH or CH₂OAc) (see, U.S. Pat. No. 4,450,254) (prepared from Nocardia); C-15-hydroxy/acyloxy (see, U.S. Pat. No. 4,364,866) (prepared by the conversion of maytansinol by Streptomyces); C-15-methoxy (see, U.S. Pat. Nos. 4,313,946 and 4,315,929) (isolated from Trewia nudiflora); C-18-N-demethyl (see, U.S. Pat. Nos. 4,362,663 and 4,322,348) (prepared by the demethylation of maytansinol by Streptomyces); and 4,5-deoxy (see, U.S. Pat. No. 4,371,533) (prepared by the titanium trichloride/LAH reduction of maytansinol).

Hemiasterlins include but are not limited to, hemiasterlin and HT1-286.

Other tubulin disrupting agents include taccalonolide A, taccalonolide B, taccalonolide AF, taccalonolide AJ, taccalonolide Al-epoxide, discodermolide, epothilone A, epothilone B, and laulimalide.

In some embodiments, a cytotoxic agent can be a topoisomerase inhibitor, such as a camptothecin. Exemplary camptothecins include, for example, camptothecin, irinotecan (also referred to as CPT-11), belotecan, (7-(2-(N-isopropylamino)ethyl)camptothecin), topotecan, 10-hydroxy-CPT, SN-38, exatecan and the exatecan analog DXd (see US20150297748). In some embodiments, provided is a conjugate wherein the cytotoxic agent is a diastereomer of exatecan. Other camptothecins are disclosed in WO1996/021666, WO00/08033, US2016/0229862 and WO2020/156189.

In some embodiments, a cytotoxic agent is a duocarmcycin, including the synthetic analogues, KW-2189 and CBI-TMI.

### Immune Modulatory Agents

In some embodiments, a Drug unit is an immune modulatory agent. An immune modulatory agent can be, for example, a TLR7 and/or TLR8 agonist, a STING agonist, a RIG-I agonist or other immune modulatory agent.

In some embodiments, a Drug unit is an immune modulatory agent, such as a TLR7 and/or TLR8 agonist. In some embodiments, a TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3. In some embodiments, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide or a benzonaphthyridine. In some embodiments, a TLR7 agonist is a non-naturally occurring compound. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20160168164, US 20150299194, US20110098248, US20100143301, and US20090047249.

In some embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In some embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, and a tetrahydropyridopyrimidine. In some embodiments, a TLR8 agonist is a non-naturally occurring compound. Examples of TLR8 agonists include motolimod, resiquimod, 3M-051, 3M-052, MCT-465, IMO-4200, VTX-763, VTX-1463.

In some embodiments, a TLR8 agonist can be any of the compounds described WO2018/170179, WO2020/056198 and WO2020056194.

Other TLR7 and TLR8 agonists are disclosed in, for example, WO2016142250, WO2017046112, WO2007024612, WO2011022508, WO2011022509, WO2012045090, WO2012097173, WO2012097177, WO2017079283, US20160008374, US20160194350, US20160289229, U.S. Pat. No. 6,043,238, US20180086755, WO2017216054, WO2017190669, WO2017202704, WO2017202703, WO20170071944, US20140045849, US20140073642, WO2014056953, WO2014076221, WO2014128189, US20140350031, WO2014023813, US20080234251, US20080306050, US20100029585, US20110092485, US20110118235, US20120082658, US20120219615, US20140066432, US20140088085, US20140275167, and US20130251673, WO2018198091, and US20170131421.

In some embodiments, an immune modulatory agent is a STING agonist. Examples of STING agonists include, for example, those disclosed in WO2020059895, WO2015077354, WO2020227159, WO2020075790, WO2018200812, and WO2020074004.

In some embodiments, an immune modulatory agent is a RIG-I agonist. Examples of RIG-I agonists include KIN1148, SB-9200, KIN700, KIN600, KIN500, KIN100, KIN101, KIN400 and KIN2000.

### Toxins

In some embodiments, a Drug unit is an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

### Radioisotopes

In some embodiments, a Drug unit is a radioactive atom. A variety of radioactive isotopes are available for the production of radioconjugates. Examples include yttrium-88, yttrium-90, technetium-99, copper-67, rhenium-188, rhenium-186, gallium-66, gallium-67, indium-11, indium-114, indium-115, lutetium-177, strontium-89, sacrarium-153, and lead-212.

### PROTACs

In some embodiments, a Drug unit is a proteolysis targeted chimera (PROTAC). PROTACs are described in, for example, Published US Application Nos. 20210015942, 20210015929, 20200392131, 20200216507, US20200199247 and US20190175612; the disclosures of which are incorporated by reference herein.

### Ligands

In some embodiments, a Drug unit includes ligands that can be bound by a Carboxyl unit, such as platinum (Pt), ruthenium (Ru), rhodium (Rh), gold (Au), silver (Ag), copper (Cu), molybdenum (Mo), titanium (Ti), or iridum (Ir); a radioisotope such as yttrium-88, yttrium-90, technetium-99, copper-67, rhenium-188, rhenium-186, gallium-66, gallium-67, indium-111, indium-114, indium-115, lutetium-177, strontium-89, sararium-153, and lead-212.

### Drug Loading

Conjugates can contain one or more Drug unit per Targeting unit. The number of Drug units per Targeting unit is referred to as drug loading. The drug loading of a Conjugate is represented by p^{load}, the average number of Drug units (drug molecules (e.g., cytotoxic agents)) per Targeting units (e.g., an antibody or antigen binding portion or non-antibody scaffold or non-antibody protein) in a conjugate. For example, if p^{load} is about 4, the average drug loading taking into account all of the Targeting units (e.g., antibodies or antigen binding portion or non-antibody scaffold or non-antibody proteins) present in the composition is about 4. In some embodiments, p^{load} ranges from about 3 to about 5, from about 3.6 to about 4.4, or from about 3.8 to about 4.2. In some embodiments, p^{load} can be about 3, about 4, or about 5. In some embodiments, p^{load} ranges from about 6 to about 8, more preferably from about 7.5 to about 8.4. In some embodiments, p^{load} can be about 6, about 7, or about 8. In some embodiments, p^{load} ranges from about 8 to about 16.

The average number of Drug units per Targeting unit (e.g., antibody or antigen binding portion or non-antibody scaffold) in a preparation may be characterized by conventional means such as UV, mass spectroscopy, Capillary Electrophoresis (CE), and HPLC. The quantitative distribution of conjugates in terms of p^{load} may also be determined. In some instances, separation, purification, and characterization of homogeneous conjugates where p^{load} is a certain value from conjugates with other drug loadings may be achieved by means such as reverse phase HPLC or Hydrophobic Interaction Chromatography (HIC) HPLC.

### Attachment of Drug-Linkers to Antibodies, Antigen Binding Portions and Other Binding Agents (Including Non-Antibody Scaffolds)

Techniques for attaching Drug unit(s) to Targeting units (such as antibodies or antigen binding portions thereof or non-antibody scaffolds) via linkers are well-known in the art. See, e.g., Alley et al., Current Opinion in Chemical Biology 2010 14:1-9; Senter, Cancer J., 2008, 14(3):154-169. In some embodiments, a Linker is first attached to a Drug unit (e.g., a cytotoxic agent(s), immune modulatory agent or other agent) and then the Drug-Linker(s) is attached to the Targeting unit (e.g., an antibody or antigen binding portion thereof or non-antibody protein scaffold). In some embodiments, a Linker(s) is first attached to a Targeting unit (e.g., an antibody or antigen binding portion thereof or non-antibody protein scaffold), and then a Drug unit is attached to a Linker. In the following discussion, the term Drug-Linker is used to exemplify attachment of Linkers or Drug-Linkers to Targeting units; the skilled artisan will appreciate that the selected attachment method can be determined according to Linker and the Drug unit. In some embodiments, a Drug unit is attached to a Targeting unit via a Linker in a manner that reduces the activity of the Drug unit until it is released from the conjugate (e.g., by hydrolysis, by proteolytic degradation or by a cleaving agent.).

Generally, a conjugate may be prepared by several routes employing organic chemistry reactions, conditions, and reagents known to those skilled in the art, including: (1) reaction of a nucleophilic group of a Targeting unit (e.g., an antibody or antigen binding portion thereof or non-antibody protein scaffold) with a bivalent Linker to form a Targeting unit-Linker intermediate via a covalent bond, followed by reaction with a Drug unit; and (2) reaction of a nucleophilic group of a Drug unit with a bivalent Linker, to form Drug-Linker, via a covalent bond, followed by reaction with a nucleophilic group of a Targeting unit. Exemplary methods for preparing conjugates via the latter route are described in U.S. Pat. No. 7,498,298, which is expressly incorporated herein by reference.

Nucleophilic groups on Targeting units such as antibodies, antigen binding portions and other binding agents (including non-antibody scaffolds) include, but are not limited to: (i)N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on Linkers including: (i) active esters such as NHS esters, HOBt esters, haloformates, and acid halides; (ii) alkyl and benzyl halides such as haloacetamides; and (iii) aldehydes, ketones, carboxyl, and maleimide groups. Certain Targeting units, such as antibodies (and antigen binding portions and other binding agents (including non-antibody scaffolds)) have reducible interchain disulfides, i.e., cysteine bridges. Antibodies (and antigen binding portions and other binding agents (including non-antibody scaffolds)) may be made reactive for conjugation with Linkers by treatment with a reducing agent such as DTT (dithiothreitol) or tricarbonylethylphosphine (TCEP), such that the antibody is fully or partially reduced. Each cysteine bridge will thus form, theoretically, two reactive thiol nucleophiles. Additional nucleophilic groups can be introduced into Targeting units such as antibodies (and antigen binding portions and other binding agents (including non-antibody scaffolds)) through modification of lysine residues, e.g., by reacting lysine residues with 2-iminothiolane (Traut's reagent), resulting in conversion of an amine into a thiol. Reactive thiol groups may also be introduced into a Targeting unit (such as an antibody and antigen binding portions and other binding agents (including non-antibody scaffolds)) by introducing one, two, three, four, or more cysteine residues (e.g., by preparing antibodies, antigen binding portions and other binding agents (including non-antibody scaffolds) comprising one or more non-native cysteine amino acid residues).

Conjugates may also be produced by reaction between an electrophilic group on a Targeting unit, such as an aldehyde or ketone carbonyl group, with a nucleophilic group on a Linker reagent. Useful nucleophilic groups on a linker reagent include, but are not limited to, hydrazide, oxime, amino, hydrazine, thiosemicarbazone, hydrazine carboxyl, and arylhydrazide. In an embodiment, an antibody (or antigen binding portion thereof or other binding agent (including non-antibody scaffolds)) is modified to introduce electrophilic moieties that are capable of reacting with nucleophilic substituents on a Linker. In another embodiment, the sugars of glycosylated antibodies may be oxidized, e.g. with periodate oxidizing reagents, to form aldehyde or ketone groups which may react with the amine group of a Linker. The resulting imine Schiff base groups may form a stable linkage, or may be reduced, e.g., by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the antibody (or antigen binding portion thereof or other binding agent (including non-antibody scaffolds)) that can react with appropriate groups on the Linker (see, e.g., Hermanson, Bioconjugate Techniques). In another embodiment, Targeting units such as antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a Linker.

Exemplary nucleophilic groups on a Drug unit, such as a cytotoxic agent, include, but are not limited to: amine, thiol, hydroxyl, hydrazide, oxime, hydrazine, thiosemicarbazone, hydrazine carboxyl, and arylhydrazide groups capable of reacting to form covalent bonds with electrophilic groups on a Linker(s) including: (i) active esters such as NHS esters, HOBt esters, haloformates, and acid halides; (ii) alkyl and benzyl halides such as haloacetamides; (iii) aldehydes, ketones, carboxyl, and maleimide groups.

In some embodiments, a Drug-Linker is attached to an interchain cysteine residue(s) of an antibody (or antigen binding portion thereof or other binding agent (including non-antibody scaffolds)). See, e.g., WO2004/010957 and WO2005/081711. In such embodiments, the Linker typically comprises a maleimide group for attachment to the cysteine residues of an interchain disulfide. In some embodiments, a Linker or Drug-Linker is attached to a cysteine residue(s) of an antibody or antigen binding portion thereof as described in U.S. Pat. No. 7,585,491 or 8,080,250. The drug loading of the resulting conjugate typically ranges from 1 to 8 or 1 to 16.

In some embodiments, a Linker or Drug-Linker is attached to a lysine or cysteine residue(s) of an antibody (or antigen binding portion thereof or other binding agent) as described in WO2005/037992 or WO2010/141566. The drug loading of the resulting conjugate typically ranges from 1 to 8.

In some embodiments, engineered cysteine residues, poly-histidine sequences, glycoengineering tags, or transglutaminase recognition sequences can be used for site-specific attachment of linkers or drug-linkers to antibodies or antigen binding portions thereof or other binding agents (including non-antibody scaffolds).

In some embodiments, a Drug-Linker(s) is attached to an engineered cysteine residue at an Fc residue other than an interchain disulfide. In some embodiments, a Drug-Linker(s) is attached to an engineered cysteine introduced into an IgG (typically an IgG1) at position 118, 221, 224, 227, 228, 230, 231, 223, 233, 234, 235, 236, 237, 238, 239, 240, 241, 243, 244, 245, 247, 249, 250, 258, 262, 263, 264, 265, 266, 267, 268, 269, 270, 271, 272, 273, 275, 276, 278, 280, 281, 283, 285, 286, 291, 292, 293, 294, 295, 296, 297, 298, 299, 300, 302, 305, 313, 318, 323, 324, 325, 327, 328, 329, 330, 331, 332, 333, 335, 336, 396, and/or 428, of the heavy chain and/or to a light chain at position 106, 108, 142 (light chain), 149 (light chain), and/or position V205, according to the EU numbering of Kabat. An exemplary substitution for site specific conjugation using an engineered cysteine is S239C (see, e.g., US 20100158909; numbering of the Fc region is according to the EU index).

In some embodiments, a Linker or Drug-Linker(s) is attached to one or more introduced cysteine residues of an antibody (or antigen binding portion thereof or other binding agent (including non-antibody scaffolds)) as described in WO2006/034488, WO2011/156328 and/or WO2016040856.

In some embodiments, an exemplary substitution for site specific conjugation using bacterial transglutaminase is N297S or N297Q of the Fc region. In some embodiments, a Linker or Drug-Linker(s) is attached to the glycan or modified glycan of an antibody or antigen binding portion or a glycoengineered antibody (or other binding agent (including non-antibody scaffolds)). See, e.g., WO2017/147542, WO2020/123425, WO2020/245229, WO2014/072482; WO2014//065661, WO2015/057066 and WO2016/022027; the disclosure of which are incorporated by reference herein.

In some embodiments, a Linker or Drug-Linker is attached to an antibody, antigen binding portion or other binding agent (including non-antibody scaffolds) via Sortase A linker. A Sortase A linker can be created by a Sortase A enzyme fusing an LPXTG recognition motif (SEQ ID NO: 5) to an N-terminal GGG motif to regenerate a native amide bond.

In some embodiments, a Linker or Drug-Linker is attached to an antibody, antigen binding portion or other binding agent (including non-antibody scaffolds) using SMARTag Technology, in which a bioorthogonal aldehyde handle is introduced through the oxidation of a cysteine residue, embedded in a specific peptide sequence (CxPxR), to an aldehyde-bearing formylglycine (fGly). This enzymatic modification is carried out by the formylglycine-generating enzyme (FGE). See, e.g., Liu et al., Methods Mol. Biol. 2033:131-147 (2019).

In some embodiments, a Linker or Drug-Linker is attached to an antibody, antigen binding portion or other binding agent (including non-antibody scaffolds) using cysteine conjugation with quaternized vinyl- and alkynyl-pyridine reagents. See, e.g., Matos et al., Angew Chem. Int. Ed. Engl. 58:6640-6644 (2019).

In other embodiments, a Linker or Drug-Linker is attached to an antibody, antigen binding portion or other binding agent (including non-antibody scaffolds) using bis-maleimide, C-lock, or K-lock methodologies.

### Pharmaceutical Formulations

Other aspects of the conjugates relate to compositions comprising active ingredients, including any of the conjugates described herein. In some embodiments, the composition is a pharmaceutical composition. As used herein, the term “pharmaceutical composition” refers to an active agent in combination with a pharmaceutically acceptable carrier accepted for use in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The preparation of a pharmacological composition that contains active ingredients dissolved or dispersed therein is well understood in the art and need not be limited based on any particular formulation. Typically such compositions are prepared as injectable either as liquid solutions or suspensions; however, solid forms suitable for rehydration, or suspensions, in liquid prior to use can also be prepared. A preparation can also be emulsified or presented as a liposome composition. A conjugate can be mixed with excipients that are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, a pharmaceutical composition can contain minor amounts of auxiliary substances such as wetting or emulsifying agents, pH buffering agents and the like which enhance or maintain the effectiveness of the active ingredient (e.g., a conjugate). The pharmaceutical compositions as described herein can include pharmaceutically acceptable salts of the components therein. Pharmaceutically acceptable salts include the acid addition salts (formed with the free amino groups of a polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain the active ingredients (e.g., a conjugate) and water, and may contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent that will be effective in the treatment of a particular disorder or condition will depend on the nature of the disorder or condition, and can be determined by standard clinical techniques.

In some embodiments, a pharmaceutical composition comprising a conjugate can be a lyophilisate.

In some embodiments, a syringe comprising a therapeutically effective amount of a conjugate is provided.

### Treatment of Cancer

In some embodiments, the conjugates as described herein can be used in a method(s) comprising administering a conjugate as described herein to a subject in need thereof, such as a subject having cancer.

In some embodiments, provided are methods of treating cancer comprising administering a conjugate In some embodiments, the subject is in need of treatment for a cancer and/or a malignancy. In some embodiments, the method is for treating a subject having a cancer or malignancy.

The methods described herein include administering a therapeutically effective amount of a conjugate to a subject having a cancer or malignancy. As used herein, the phrases “therapeutically effective amount”, “effective amount” or “effective dose” refer to an amount of a conjugate that provides a therapeutic benefit in the treatment of, management of or prevention of relapse of a cancer or malignancy, e.g., an amount that provides a statistically significant decrease in at least one symptom, sign, or marker of a tumor or malignancy. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The terms “cancer” and “malignancy” refer to an uncontrolled growth of cells which interferes with the normal functioning of the bodily organs and systems. A cancer or malignancy may be primary or metastatic, i.e. that is it has become invasive, seeding tumor growth in tissues remote from the original tumor site. A “tumor” refers to an uncontrolled growth of cells which interferes with the normal functioning of the bodily organs and systems. A subject that has a cancer is a subject having objectively measurable cancer cells present in the subject's body. Included in this definition are benign tumors and malignant cancers, as well as potentially dormant tumors and micro-metastases. Cancers that migrate from their original location and seed other vital organs can eventually lead to the death of the subject through the functional deterioration of the affected organs. Hematologic malignancies (hematopoietic cancers), such as leukemias and lymphomas, are able to, for example, out-compete the normal hematopoietic compartments in a subject, thereby leading to hematopoietic failure (in the form of anemia, thrombocytopenia and neutropenia) ultimately causing death.

Examples of cancers include, but are not limited to, carcinomas, lymphomas, blastomas, sarcomas, and leukemias. More particular examples of such cancers include, but are not limited to, basal cell cancer, biliary tract cancer, bladder cancer, bone cancer, brain and CNS cancer, breast cancer (e.g., triple negative breast cancer), cancer of the peritoneum, cervical cancer; cholangiocarcinoma, choriocarcinoma, chondrosarcoma, colon and rectum cancer (colorectal cancer), connective tissue cancer, cancer of the digestive system, endometrial cancer, esophageal cancer, eye cancer, cancer of the head and neck, gastric cancer (including gastrointestinal cancer and stomach cancer), glioblastoma (GBM), hepatic cancer, hepatoma, intra-epithelial neoplasm, kidney or renal cancer (e.g., clear cell cancer), larynx cancer, leukemia, liver cancer, lung cancer (e.g., small-cell lung cancer, non-small cell lung cancer, adenocarcinoma of the lung, and squamous cancer of the lung), lymphoma including Hodgkin's and non-Hodgkin's lymphoma, melanoma, mesothelioma, myeloma, neuroblastoma, oral cavity cancer (e.g., lip, tongue, mouth, and pharynx), ovarian cancer, pancreatic cancer, prostate cancer, retinoblastoma, rhabdomyosarcoma, cancer of the respiratory system, salivary gland cancer, sarcoma, skin cancer, squamous cell cancer, testicular cancer, thyroid cancer, uterine or endometrial cancer, uterine serious cancer, cancer of the urinary system, vulval cancer; as well as other carcinomas and sarcomas, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia), chronic lymphocytic leukemia (CLL), acute lymphoblastic leukemia (ALL), Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome.

It is contemplated that the methods herein reduce tumor size or tumor burden in the subject, and/or reduce metastasis in the subject. In various embodiments, tumor size in the subject is decreased by about 25-50%, about 40-70% or about 50-90% or more. In various embodiments, the methods reduce the tumor size by 10%, 20%, 30% or more. In various embodiments, the methods reduce tumor size by 10%, 15%, 20%, 25%, 30%, 35%, 40%, 45%, 50%, 55%, 60%, 65%, 70%, 75%, 80%, 85%, 90%, 95% or 100%.

In some embodiments, the subject is in need of treatment for a cancer and/or a malignancy with an anti-FOLRI conjugate. In a specific embodiment, the anti-FOLRI conjugate contains antibody F131 (VH SEQ ID NO: 26 and VL SEQ ID NO: 27). In some embodiments, the subject is in need of treatment for a FOLR1+ cancer or a FOLR1+ malignancy, such as for example, lung cancer, non-small cell lung cancer, ovarian cancer, breast cancer, uterine cancer, cervical cancer, endometrial cancer, pancreatic cancer, and renal cell cancer. In some embodiments, the method is for treating a subject having a FOLR1+ cancer or malignancy. In some embodiments, the method is for treating lung cancer in a subject. In some embodiments, the method is for treating non-small cell lung cancer in a subject. In some embodiments, the method is for treating breast cancer in a subject. In some embodiments, the method is for treating ovarian cancer in a subject. In some embodiments, the method is for treating cervical cancer in a subject. In some embodiments, the method is for treating endometrial cancer in a subject. In some embodiments, the method is for treating renal cell cancer in a subject. In some embodiments, the method is for treating uterine cancer in a subject. In some embodiments, the method is for treating pancreatic cancer in a subject.

As used herein, a “subject” refers to a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate, e.g., a human. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various cancers. In addition, the methods described herein can be used to treat domesticated animals and/or pets. A subject can be male or female. In certain embodiments, the subject is a human.

In some embodiments, a subject can be one who has been previously diagnosed with or identified as suffering from a cancer and in need of treatment, but need not have already undergone treatment for the cancer. In some embodiments, a subject can also be one who has not been previously diagnosed as having a cancer in need of treatment. In some embodiments, a subject can be one who exhibits one or more risk factors for a condition or one or more complications related to a cancer or a subject who does not exhibit risk factors. A “subject in need” of treatment for a cancer particular can be a subject having that condition or diagnosed as having that condition. In other embodiments, a subject “at risk of developing” a condition refers to a subject diagnosed as being at risk for developing the condition or at risk for having the condition again.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, reduction in cancer cells in the subject, alleviation of one or more symptom(s), diminishment of extent of the deficit, stabilized (i.e., not worsening) state of a cancer or malignancy, delay or slowing of tumor growth and/or metastasis, and an increased lifespan as compared to that expected in the absence of treatment. As used herein, the term “administering,” refers to providing a conjugate as described herein to a subject by a method or route which results in binding of the conjugate to cancer cells or malignant cells. Similarly, a pharmaceutical composition comprising a conjugate as described herein can be administered by any appropriate route which results in an effective treatment in the subject.

The dosage ranges for a conjugate depend upon the potency, and encompass amounts large enough to produce the desired effect e.g., slowing of tumor growth or a reduction in tumor size. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dosage ranges from 0.5 mg/kg body weight to 15 mg/kg body weight. In some embodiments, the dose range is from 0.5 mg/kg body weight to 5 mg/kg body weight. Alternatively, the dose range can be titrated to maintain serum levels between 1 μg/mL and 1000 μg/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 12 mg/kg or more.

Administration of the doses recited above can be repeated. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg. In some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 0.1 mg/kg to about 12 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be from about 1 mg/kg to about 12 mg/kg. In some embodiments, a dose can be from about 1 mg/kg to about 10 mg/kg.

In some embodiments, a dose can be administered intravenously. In some embodiments, an intravenous administration can be an infusion occurring over a period of from about 10 minutes to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing a conjugate can be administered in a unit dose. The term “unit dose” when used in reference to a pharmaceutical composition refers to physically discrete units suitable as unitary dosage for the subject, each unit containing a predetermined quantity of active material (e.g., conjugate), calculated to produce the desired therapeutic effect in association with the required physiologically acceptable diluent, i.e., carrier, or vehicle.

### Treatment of Autoimmune Disease

In some embodiments, the conjugates as described herein can be used in a method(s) comprising administering a conjugate to a subject in need thereof, such as a subject having an autoimmune disease.

In some embodiments, provided are methods of treating an autoimmune disease comprising administering a conjugate as described herein. In some embodiments, the subject is in need of treatment for an autoimmune disease. The methods described herein include administering a therapeutically effective amount of a conjugate to a subject having an autoimmune disease. As used herein, the phrase “therapeutically effective amount”, “effective amount” or “effective dose” refers to an amount of a conjugate as described herein that provides a therapeutic benefit in the treatment of, management of or prevention of relapse of an autoimmune disease, e.g., an amount that provides a statistically significant decrease in at least one symptom, sign, or marker of an autoimmune disease. Determination of a therapeutically effective amount is well within the capability of those skilled in the art. Generally, a therapeutically effective amount can vary with the subject's history, age, condition, sex, as well as the seventy and type of the medical condition in the subject, and administration of other pharmaceutically active agents.

The term “autoimmune disease” refers to an immunological disorder characterized by inappropriate activation of immune cells (e.g., lymphocytes or dendritic cells), that interferes with the normal functioning of the bodily organs and systems. Examples of autoimmune disease include, but are not limited to, rheumatoid arthritis, psoriatic arthritis, autoimmune demyelinative diseases (e.g., multiple sclerosis, allergic encephalomyelitis), endocrine ophthalmopathy, uveoretinitis, systemic lupus erythematosus, myasthenia gravis, Grave's disease, glomerulonephritis, autoimmune hepatological disorder, inflammatory bowel disease (e.g., Crohn's disease), anaphylaxis, allergic reaction, Sjogren's syndrome, type I diabetes mellitus, primary biliary cirrhosis, Wegener's granulomatosis, fibromyalgia, polymyositis, dermatomyositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia areata, pemphigoid, scleroderma, progressive systemic sclerosis, CREST syndrome (calcinosis, Raynaud's phenomenon, esophageal dysmotility, sclerodactyl), and telangiectasia), male and female autoimmune infertility, ankylosing spondolytis, ulcerative colitis, mixed connective tissue disease, polyarteritis nodosa, systemic necrotizing vasculitis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, toxic epidermal necrolysis, Alport's syndrome, alveolitis, allergic alveolitis, fibrosing alveolitis, interstitial lung disease, erythema nodosum, pyoderma gangrenosum, transfusion reaction, Takayasu's arteritis, polymyalgia rheumatica, temporal arteritis, schistosomiasis, giant cell arteritis, ascariasis, aspergillosis, Samter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, cardiomyopathy, Eaton-Lambert syndrome, relapsing polychondritis, cryoglobulinemia, Waldenstrom's macroglobulemia, Evan's syndrome, and autoimmune gonadal failure.

In some embodiments, the methods described herein encompass treatment of disorders of B lymphocytes (e.g., systemic lupus erythematosus, Goodpasture's syndrome, rheumatoid arthritis, and type I diabetes), Th1-lymphocytes (e.g., rheumatoid arthritis, multiple sclerosis, psoriasis, Sjorgren's syndrome, Hashimoto's thyroiditis, Grave's disease, primary biliary cirrhosis, Wegener's granulomatosis, tuberculosis, or graft versus host disease), or Th2-lymphocytes (e.g., atopic dermatitis, systemic lupus erythematosus, atopic asthma, rhinoconjunctivitis, allergic rhinitis, Omenn's syndrome, systemic sclerosis, or chronic graft versus host disease). Generally, disorders involving dendritic cells involve disorders of Th1-lymphocytes or Th2-lymphocytes.

As used herein, a “subject” refers to a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate, e.g., a human. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various autoimmune diseases. In addition, the methods described herein can be used to treat domesticated animals and/or pets. A subject can be male or female. In certain embodiments, the subject is a human.

In some embodiments, a subject can be one who has been previously diagnosed with or identified as suffering from an autoimmune disease and in need of treatment, but need not have already undergone treatment for the autoimmune disease. In some embodiments, a subject can also be one who has not been previously diagnosed as having an autoimmune disease in need of treatment. In some embodiments, a subject can be one who exhibits one or more risk factors for a condition or one or more complications related to an autoimmune disease or a subject who does not exhibit risk factors. A “subject in need” of treatment for an autoimmune disease particular can be a subject having that condition or diagnosed as having that condition. In other embodiments, a subject “at risk of developing” a condition refers to a subject diagnosed as being at risk for developing the condition or at risk for having the condition again (e.g., an autoimmune disease).

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, reduction in autoimmune cells in the subject, alleviation of one or more symptom(s), diminishment of extent of the deficit, stabilized (i.e., not worsening) state of an autoimmune disease, delay or slowing of progression of an autoimmune disease, and an increased lifespan as compared to that expected in the absence of treatment. As used herein, the term “administering,” refers to providing a conjugate as described herein to a subject by a method or route which results in binding of the conjugate to target autoimmune cells. Similarly, a pharmaceutical composition comprising a conjugate as described herein can be administered by any appropriate route which results in an effective treatment in the subject.

The dosage ranges for a conjugate depend upon the potency, and encompass amounts large enough to produce the desired effect e.g., slowing of progression of an autoimmune disease or a reduction of symptoms. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dosage ranges from 0.5 mg/kg body weight to 15 mg/kg body weight. In some embodiments, the dose range is from 0.5 mg/kg body weight to 5 mg/kg body weight. Alternatively, the dose range can be titrated to maintain serum levels between 1 μg/mL and 1000 μg/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 12 mg/kg or more.

Administration of the doses recited above can be repeated. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg. In some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 0.1 mg/kg to about 12 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be from about 1 mg/kg to about 12 mg/kg. In some embodiments, a dose can be from about 1 mg/kg to about 10 mg/kg.

In some embodiments, a dose can be administered intravenously. In some embodiments, an intravenous administration can be an infusion occurring over a period of from about 10 minutes to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing a conjugate thereof can be administered in a unit dose. The term “unit dose” when used in reference to a pharmaceutical composition refers to physically discrete units suitable as unitary dosage for the subject, each unit containing a predetermined quantity of active material (e.g., a conjugate), calculated to produce the desired therapeutic effect in association with the required physiologically acceptable diluent, i.e., carrier, or vehicle.

In some embodiments, a conjugate, or a pharmaceutical composition of any of these, is administered with an immunosuppressive therapy. In some embodiments, provided is a method of improving treatment outcome in a subject receiving immunosuppressive therapy. The method generally includes administering an effective amount of an immunosuppressive therapy to the subject having an autoimmune disorder; and administering a therapeutically effective amount of a conjugate or a pharmaceutical composition thereof to the subject, wherein the conjugate specifically binds to target autoimmune cells; wherein the treatment outcome of the subject is improved, as compared to administration of the immunotherapy alone. In some embodiments, the conjugate thereof as described herein. In some embodiments, an improved treatment outcome is a decrease in disease progression, an alleviation of one or more symptoms, or the like.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### Abbreviations

Boc₂O: di-tert-butyl dicarbonate
Bu₄NBr: Tetrabutylammonium bromide
DCM: dichloromethane
DEA: Diethanolamine
DEAD: diethyl azodicarboxylate
DIPEA: N,N-diisopropylethylamine
DMAP: 4-(Dimethylamino)pyridine
DMF: N,N-dimethylformamide
DMTMM: 4-(4,6-dimethoxy-1,3,5-triazin-2-yl)-4-methyl-morpholinium chloride
DMSO: dimethylsulfoxide
EEDQ: N-Ethoxycarbonyl-2-ethoxy-1,2-dihydroquinoline
ESI: electrospray ionization
HATU: 1-[bis(dimethylamino)methylene]-1H-1,2,3-triazolo[4,5-b]pyridinium 3-oxidhexafluorophosophate)
HOBt: hydroxylbenzotriazole
LCMS: liquid chromatography-mass spectrometry
MeCN: acetonitrile
MeOH: methanol
m-CPBA: meta-chloroperoxybenzoic acid
MTBE: Methyl tert-butyl ether
NMR: nuclear magnetic resonance spectroscopy
Ph₃CCl: Triphenylmethyl chloride
PNPC: bis(4-nitrophenyl) carbonate
PPh₃: triphenylphosphine
TFA: trifluoroacetic acid
THF: tetrahydrofuran
TLC: thin-layer chromatography
TsOH: p-Toluenesulfonic acid

### General Methods

¹H NMR and other NMR spectra were recorded on Bruker AVIII 400 or Bruker AVIII 500. The data were processed with Nuts software or MestReNova software, measuring proton shifts in parts per million (ppm) downfield from an internal standard tetramethyl silane.

HPLC-MS measurement was run on Agilent 1200 HPLC/6100 SQ System using the following conditions:
Method A: Mobile Phase: A: Water (0.01% TFA) B: acetonitrile (0.01% TFA); Gradient Phase: 5% of B increasing to 95% of B in 15 min; Flow Rate: 1.0 mL/min; Column: XBridge C18, 4.6*150 mm, 3.5 um; Column Temperature: 40° C. Detectors: ADC ELSD, DAD (214 nm and 254 nm), ES-API.
Method B: Mobile Phase: A: Water (0.01% TFA) B: acetonitrile (0.01% TFA); Gradient Phase: 5% of B increasing to 95% of B in 15 min; Flow Rate: 1.0 mL/min; Column: SunFire C18, 4.6*150 mm, 3.5 μm; Column Temperature: 45° C. Detectors: ADC ELSD, DAD (214 nm and 254 nm), ES-API.
Method C: Mobile Phase: A: Water (10 mM NH₄HCO₃) B: acetonitrile; Gradient Phase: 5% to 95% of B in 15 min; Flow Rate: 1.0 mL/min; Column: XBridge C18, 4.6*150 mm, 3.5 μm; Column Temperature: 40° C. Detectors: ADC ELSD, DAD (214 nm and 254 nm), MSD (ES-API).

LCMS measurement was run on Agilent 1200 HPLC/6100 SQ System using the following conditions:
Method A: Mobile Phase: A: Water (0.01% TFA) B: acetonitrile (0.01% TFA); Gradient Phase: 5% of B increasing to 95% of B in 3 min; Flow Rate: 1.8-2.3 mL/min; Column: SunFire C18, 4.6*50 mm, 3.5 μm; Column Temperature: 50° C. Detectors: ADC ELSD, DAD (214 nm and 254 nm), ES-API.
Method B: Mobile Phase: A: Water (10 mM NH₄HCO₃) B: Acetonitrile; Gradient Phase: 5% to 95% of B in 3 min; Flow Rate: 1.8-2.3 mL/min; Column: XBridge C18, 4.6*50 mm, 3.5 μm; Column Temperature: 50° C. Detectors: ADC ELSD, DAD (214 nm and 254 nm), MSD (ES-API).

Preparative high pressure liquid chromatography (Prep-HPLC) was run on Gilson 281 using the following conditions:
Method A: Waters SunFire 10 μm C18 column (100 \acute{Å}, 250×19 mm). Solvent A was water/0.01% trifluoroacetic acid (TFA) and solvent B was acetonitrile. The elution condition was a linear gradient increase of solvent B from 5% to 100% over a time period of 20 minutes at a flow rate of 30 mL/min.
Method B: Waters SunFire 10 μm C18 column (100 \acute{Å}, 250×19 mm). Solvent A was water/0.05% formic acid (FA) and solvent B was acetonitrile. The elution condition was a linear gradient increase of solvent B from 5% to 100% over a time period of 20 minutes at a flow rate of 30 mL/min.
Method C: Waters Xbridge 10 μm C18 column (100 \acute{Å}, 250×19 mm). Solvent A was water/10 mM ammonium bicarbonate (NH₄HCO₃) and solvent B was acetonitrile. The elution condition was a linear gradient increase of solvent B from 5% to 100% over a time period of 20 minutes at a flow rate of 30 mL/min.

Flash chromatography was performed on instrument of Biotage, with Agela Flash Column silica-CS; Reverse phase flash chromatography was performed on instrument of Biotage, with Boston ODS or Agela C18.

### Example 1: Preparation of a Sugar Unit

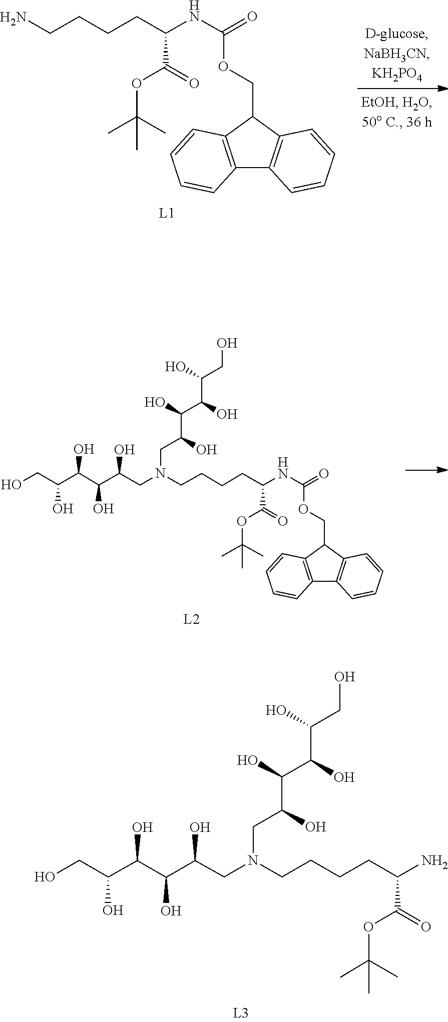 [SMILES: CC(C)(C)OC(=O)[C@H](CCCCN)NC(=O)OCC1c2ccccc2-c2ccccc21 ; CC(C)(C)OC(=O)[C@H](CCCCN(C[C@H](O)[C@@H](O)[C@H](O)[C@H](O)CO)C[C@H](O)[C@@H](O)[C@H](O)[C@H](O)CO)NC(=O)OCC1c2ccccc2-c2ccccc21 ; CC(C)(C)OC(=O)[C@@H](N)CCCCN(C[C@H](O)[C@@H](O)[C@H](O)[C@H](O)CO)C[C@H](O)[C@@H](O)[C@H](O)[C@H](O)CO]

A Sugar unit was prepared as follows:

Step 1 A reaction mixture of compound L1 (5 g, 10.846 mmol), D-glucose (19.54 g, 108.460 mmol), NaBH₃CN (5.45 g, 86.768 mmol) and potassium dihydrogen phosphate (0.379 mL, 6.508 mmol) in water (40 mL) and ethanol (65 mL) was stirred at 50° C. under N₂ for 36 hr, until the reaction was complete as indicated by LCMS. The solvents were evaporated, and the residue was purified by C18 reversed-phase chromatography to give the desired product L2 (3.5 g, 4.649 mmol, 42.86%). LCMS (M+H)⁺=753.0;

1H NMR (400 MHz, DMSO) δ 7.90 (d, J=7.5 Hz, 2H), 7.74-7.64 (m, 2H), 7.44-7.32 (m, 4H), 4.58-4.21 (m, 8H), 4.14-3.74 (m, 4H), 3.68-3.41 (m, 8H), 2.85-2.56 (m, 2H), 1.69-1.28 (m, 15H). 13C NMR (100 MHz, DMSO) δ 171.53, 156.10, 143.77, 140.70, 127.62, 127.04, 125.24, 120.10, 80.47, 80.42, 71.66, 71.58, 71.34, 70.18, 65.53, 63.51, 63.36, 54.48, 54.41, 46.63, 27.65, 23.14, 22.38.

Step 2 To a solution of L2 (200 mg, 0.266 mmol) in THF (2 mL) was added the diethylamine (38.86 mg, 0.531 mmol). The reaction mixture was stirred at room temperature for 2 hr. A sample was taken from the reaction mixture, and the LCMS result showed the desired product was found and the starting material was consumed completely. The solvents were evaporated, and the residue was purified by C18 reversed-phase chromatography to give the desired product L3 (120 mg), LCMS (M+H)⁺=531.1.

### Example 2: Preparation of an Exemplary Polar Group

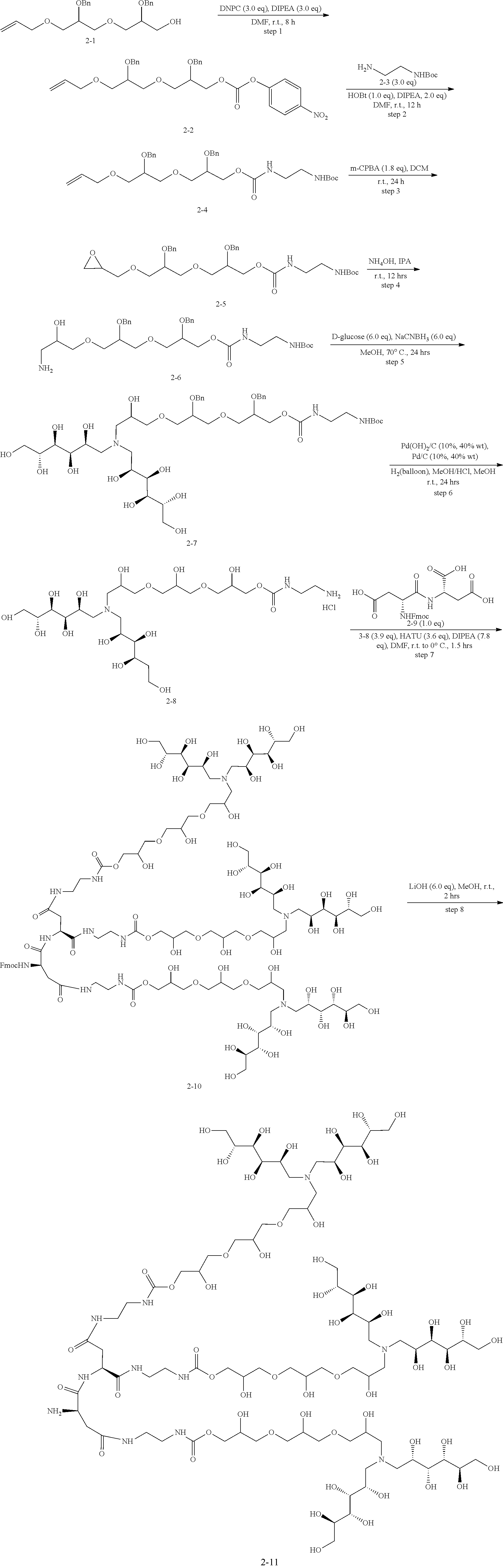

An Exemplary Polar Group was prepared as follows:

### Step 1

To a solution of 2-1 (600 mg, 1.554 mmol) in DMF (12 mL) was added DIPEA (602.4 mg, 4.661 mmol), followed by 4,4′-dinitrodiphenyl carbonate (1.42 g, 4.661 mmol), then the resulting mixture was stirred at room temperature for 8 hrs until 2-1 was consumed as detected by LCMS. The reaction solution was directly used in the next step without a work-up procedure.

### Step 2

To the above reaction mixture was added HOBt (210 mg, 1.554 mmol), DIPEA (401.7 mg, 3.108 mmol) and 2-3 (746.5 mg, 4.662 mmol) successively, and the resulting mixture was stirred at room temperature for 6 hrs until 2-2 was consumed as detected by LCMS. The reaction mixture was diluted with ethyl acetate (180 mL) and washed with saturated NaHCO₃ (aq, 45 mL×3), dried over anhydrous Na₂SO₄, filtered and concentrated to dryness under reduced pressure. The crude product was purified with column chromatography (silica, 0-80% ethyl acetate in petroleum ether) affording 2-4 (893 mg, 1.56 mmol, 100.4% over 2 steps) as a pale yellow oil.

### Step 3

To a solution of 2-4 (890 mg, 1.555 mmol) in DCM (10 mL) was added a solution of m-CPBA (483 mg, 2.80 mmol) in DCM (10 mL) dropwise at room temperature. The resulting mixture was stirred at this temperature for 24 hours until 2-4 was consumed, and the reaction was quenched with saturated Na₂S₂O₃ (aq, 10 mL) and NaHCO₃ (aq., 10 mL). The reaction mixture was stirred for 30 mins, and then diluted with DCM (50 mL). The organic phase was washed with a mixture solution (20 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-100% ethyl acetate in petroleum ether) affording 2-5 (790 mg, 1.343 mmol, 86.4%) as a pale yellow oil. Purity=90%-95%.

### Step 4

To a solution of 2-5 (790 mg, 1.343 mmol) in isopropyl alcohol (110 mL) was added ammonia (110 mL) dropwise at room temperature, and the resulting mixture was stirred at this temperature for 12 hours until 2-5 was consumed. The reaction mixture was concentrated to dryness under reduced pressure to afford 2-6 (801.2 mg, 1.323 mmol, 98.6%) as a yellow oil, which was used in the next step without further purification. Purity=90%-95%.

### Step 5

A mixture of 2-6 (801.2 mg, 1.324 mmol), D-glucose (1.43 g, 7.937 mmol) and NaCNBH₃ (499.2 mg, 7.94 mmol) in anhydrous MeOH (21 mL) was stirred at 70° C. for 24 hrs until most of 2-6 was consumed and 2-7 was detected by LCMS. The reaction mixture was cooled down to room temperature, filtered and concentrated under reduced pressure to give the crude product, which was purified by reverse phase liquid chromatography to give 2-7 (1.2 g, 1.29 mmol, 97.1%) as a colorless oil. Purity=85%-90%.

### Step 6

A mixture of 2-7 (1.2 g, 1.286 mmol), Pd(OH)₂/C (10%, 250 mg) and Pd/C (10%, 250 mg) in HCl/MeOH (4M, 25 mL), MeOH (25 mL) was stirred under hydrogen atmosphere (balloon) for 24 h at room temperature until 2-7 was completely converted into 2-8. The reaction mixture was filtered through a celite pad, and the filtrate was concentrated to dryness under reduced pressure to afford 2-8 (886 mg, 1.285 mmol, 100.0%) as an off-white solid, which was used in the next step without further purification.

### Step 7

A solution of 2-9 (104.9 mg, 0.223 mmol) and HATU (305.4 mg, 0.803 mmol) in anhydrous DMF (3 mL) was stirred at room temperature for 15 mins, then it was stirred in an ice bath. A solution of 2-8 (600 mg, 0.870 mmol) in anhydrous DMF (3 mL) was added dropwise, followed by DIPEA (225 mg, 1.74 mmol). The resulting mixture was stirred in the ice bath for 1 h until most of 2-9 was consumed. The reaction mixture was purified by reverse phase liquid chromatography to give 2-10 (269.8 mg, 0.113 mmol, 50.9%) as a white solid. Purity=90%-95%. ¹H NMR (400 MHz, DMSO-d₆) δ 7.87 (d, J=7.6 Hz, 2H), 7.64 (d, J=7.2 Hz, 2H), 7.47 (t, J=7.6 Hz, 2H), 7.42-7.32 (m, 2H), 4.65-4.57 (m, 1H), 4.53-4.36 (m, 3H), 4.33-4.13 (m, 10H), 4.08-3.87 (m, 12H), 3.84-3.69 (m, 18H), 3.67-3.35 (m, 53H), 3.30-3.07 (m, 12H), 2.77-2.32 (m, 4H).

### Step 8

To a solution of 2-10 (100 mg, 0.0421 mmol) in MeOH (3 mL) and H₂O (1 mL) was added LiOH·H₂O (10.6 mg, 0.252 mmol), and the mixture was stirred at room temperature for 2 hrs until 2-10 was consumed. The reaction solution was neutralized with 1N HCl to pH=7, and concentrated under reduced pressure to give a crude product, which was dissolved in H₂O (15 mL) and washed with hexane (10 mL×3). The aqueous phase was concentrated to dryness under reduced pressure to afford 2-11 (74.6 mg, 0.0346 mmol, 82.3%) as a colorless oil, which was used in the next step without further purification.

### Example 3: Preparation of an Exemplary Polar Group

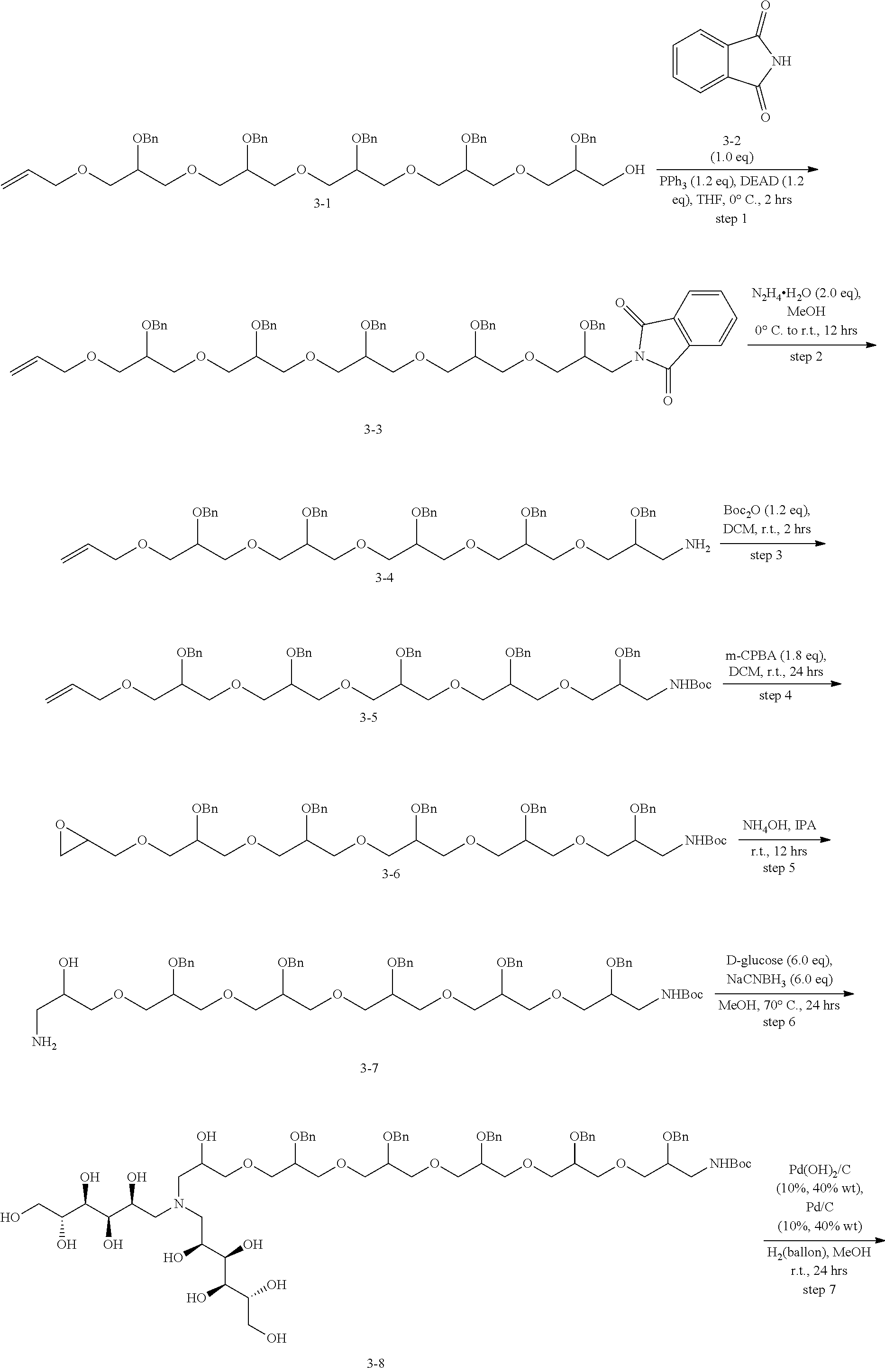

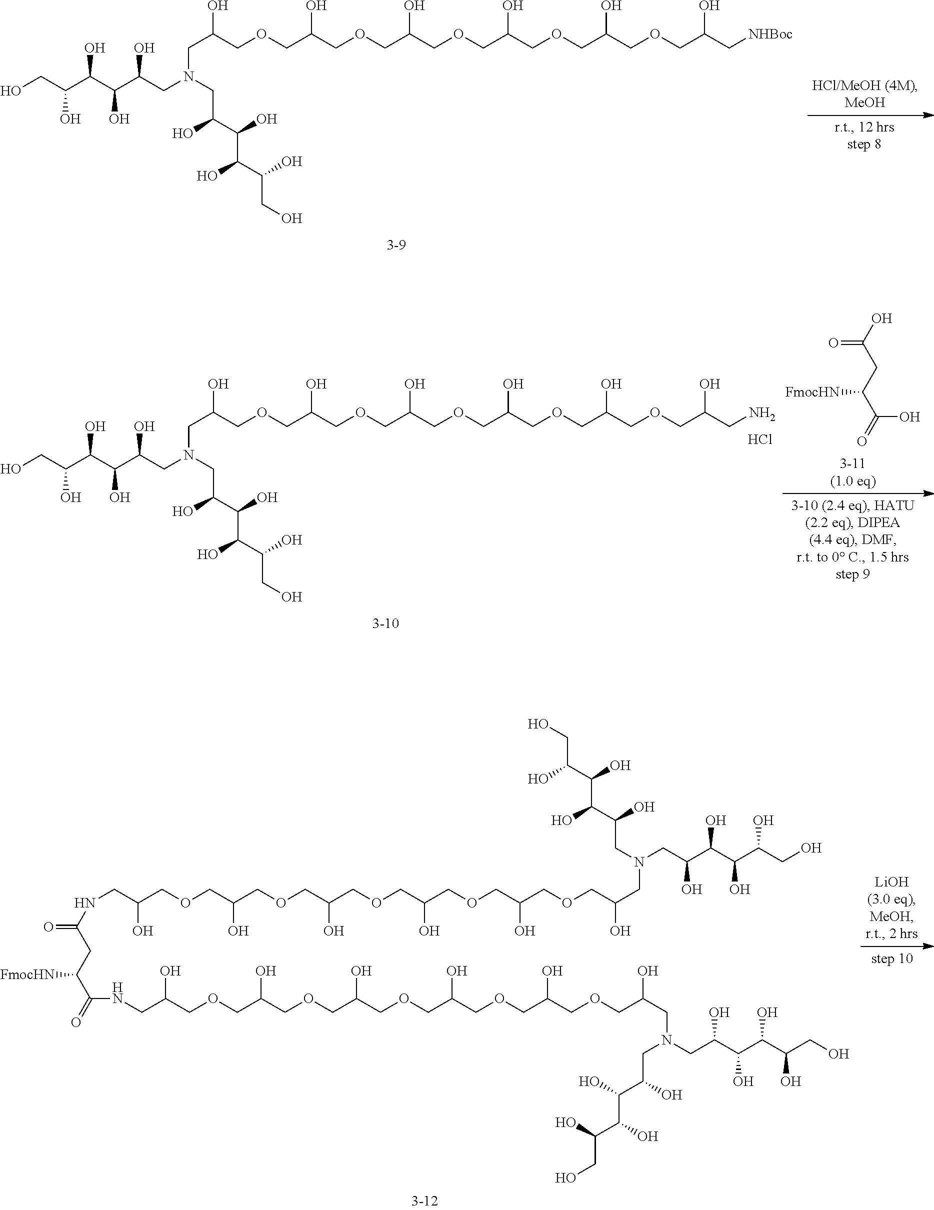

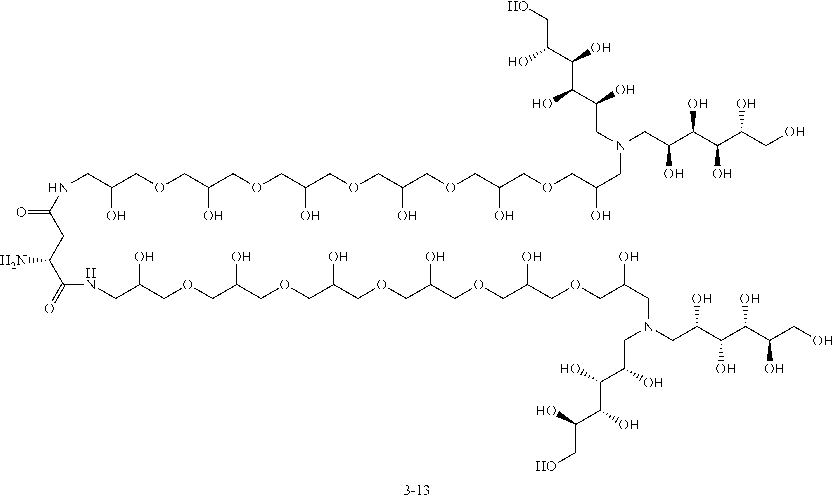 [SMILES: N[C@H](CC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

An Exemplary Polar Group was prepared as follows:

### Step 1

To a solution of 3-2 (217.6 mg, 1.48 mmol) and PPh₃ (465.5 mg, 1.776 mmol) in THF (8 mL) was added a solution of 3-1 (1.3 g, 1.48 mmol) in THF (4 mL) and the mixture was stirred in an ice bath. A solution of DEAD (309.3 mg, 1.776 mmol) in THF (1 mL) was added to the above solution and the resulting mixture was allowed to warm to r.t. and stirred for 2 hrs until 3-1 was consumed by TLC. The reaction was quenched with water (1 mL), and the reaction was concentrated under reduced pressure to give the crude product, which was purified with column chromatography (silica, 0-60% ethyl acetate in petroleum ether) to afford 3-3 (1.307 g, 1.297 mmol, 87.7%) as a white solid. Purity=90%-95%. ¹H NMR (400 MHz, CDCl₃) δ 7.77 (dd, J=5.6, 3.2 Hz, 2H), 7.66 (dd, J=5.6, 3.2 Hz, 2H), 7.34-7.20 (m, 20H), 7.19-7.15 (m, 2H), 7.12-7.04 (m, 3H), 5.95-5.81 (m, 1H), 5.29-5.22 (m, 1H), 5.15 (d, J=10.4 Hz, 1H), 4.69-4.63 (m, 9H), 4.51 (d, J=12.0 Hz, 1H), 3.99-3.95 (m, 2H), 3.93-3.83 (m, 2H), 3.76-3.69 (m, 5H), 3.60-3.52 (m, 18H).

### Step 2

To a solution of 3-3 (1.472 g, 1.46 mmol) in MeOH (10 mL) was added N₂H₄·H₂O (146.3 mg, 2.92 mmol) in an ice bath, the resulting mixture was allowed to warm to r.t. and stirred for 10 hrs until 3-3 was consumed by TLC. The reaction mixture was concentrated to dryness under reduced pressure to give the crude product, which was dissolved in ethyl acetate (20 mL) and filtered. The filtrate was concentrated under reduced pressure to afford 3-4 (1.23 g, 1.402 mmol, 95.9%) as a colorless oil, which was used in the next step without further purification.

### Step 3

To a solution of 3-4 (1.23 g, 1.40 mmol) in DCM (8 mL) was added Boc₂O (367 mg. 1.68 mmol) at room temperature, and the reaction mixture was stirred at this temperature for 2 hrs until 3-4 was consumed and 3-5 was detected by LCMS. The reaction was concentrated under reduced pressure to give the crude product, which was purified with column chromatography (silica, 0-40% ethyl acetate in petroleum ether) affording 3-5 (1.23 g, 1.26 mmol, 89.8%) as a colorless oil. Purity=90%-95%.

### Step 4

To a solution of 3-5 (800 mg, 0.818 mmol) in DCM (5 mL) was added a solution of m-CPBA (254 mg, 1.473 mmol) in DCM (5 mL) at room-temperature, and the mixture was stirred for 24 hrs until 3-5 was consumed by TLC. The reaction was quenched by adding sat. Na₂S2O₃ (5 mL) and sat. NaHCO₃ (5 mL), and the resulting mixture was stirred for 30 mins before diluting with DCM (60 mL). The organic phase was sequencely washed with sat. Na₂S2O₃ (20 mL) and sat. NaHCO₃ (20 mL), dried over anhydrous Na₂SO₄, filtered and concentrated to dryness under reduced pressure. The crude product was purified with column chromatography (silica, 0-60% Ethyl acetate in petroleum ether) to afford 3-6 (689 mg, 0.693 mmol, 84.8%) as a colorless oil. Purity=90%-95%.

### Step 5

To a solution of 3-6 (689 mg, 0.693 mmol) in isopropyl alcohol (56 mL) was added ammonia (43 mL) dropwise at room temperature, and the resulting mixture was stirred at this temperature for 12 hours until 3-6 was consumed. The reaction mixture was concentrated to dryness under reduced pressure to afford 3-7 (698.7 mg, 0.691 mmol, 99.7%) as a yellow oil, which was used in the next step without further purification. Purity=90%-95%.

### Step 6

A mixture of 3-7 (983 mg, 0.973 mmol), D-glucose (1.05 g, 5.836 mmol) and NaCNBH₃ (366.7 mg, 5.836 mmol) in anhydrous MeOH (15 mL) was stirred at 70° C. for 24 hrs until most of 3-7 was consumed and 3-8 was detected by LCMS. The reaction mixture was cooled down to room temperature, filtered and concentrated under reduced pressure to give the crude product, which was purified by reverse phase liquid chromatography to give 3-8 (889 mg, 0.664 mmol, 68.2%) as a colorless oil. Purity=90%-95%.

### Step 7

A mixture of 3-8 (270 mg, 0.202 mmol), Pd(OH)₂/C (10%, 120 mg) and Pd/C (10%, 120 mg) in MeOH (25 mL) was stirred under hydrogen atmosphere (ballon) for 24 h at room temperature until 3-8 was completely converted into 3-9. The reaction mixture was filtered through a celite pad, and the filtrate was concentrated to dryness under reduced pressure to afford 3-9 (138.2 mg, 0.156 mmol, 77.0%) as an off-white solid, which was used in the next step without further purification.

### Step 8

A solution of 3-9 (138 mg, 0.155 mmol) in HCl/MeOH (4M, 3 mL) and MeOH (3 mL) was stirred at room temperature for 12 hours until 3-9 was completely converted into 3-10. The reaction mixture was concentrated to dryness under reduced pressure to afford 3-10 (128 mg, 0.155 mmol, 100%) as an off-white solid, which was used in the next step without further purification.

### Step 9

A solution of 3-11 (16.5 mg, 0.0465 mmol) and HATU (38.9 mg, 0.102 mmol) in anhydrous DMF (2 mL) was stirred at room temperature for 15 mins, then it was stirred in an ice bath. A solution of 3-10 (92 mg, 0.112 mmol) in anhydrous DMF (2 mL) was added dropwise, followed by DIPEA (26.5 mg, 0.205 mmol). The resulting mixture was stirred in the ice bath for 1 h until most of 3-11 was consumed. The reaction mixture was purified by reverse phase liquid chromatography to give 3-12 (14 mg, 0.00738 mmol, 15.9%) as a colorless oil.

### Step 10

To a solution of 3-12 (14 mg, 0.00738 mmol) in MeOH (2 mL) was added LiOH·H₂O (2 mg, 0.0442 mmol), and the mixture was stirred at room temperature for 2 hrs until 3-12 was consumed. The reaction solution was neutralized with 1N HCl to pH=7, and concentrated under reduced pressure to give a crude product, which was dissolved in H₂O (5 mL) and washed with hexane (2 mL×3). The aqueous phase was concentrated to dryness under reduced pressure to afford 3-13 (12.4 mg, 0.0074 mmol, 100%) as a white solid, which was used in the next step without further purification.

### Example 4: Preparation of an Exemplary Polar Group

### Step 1

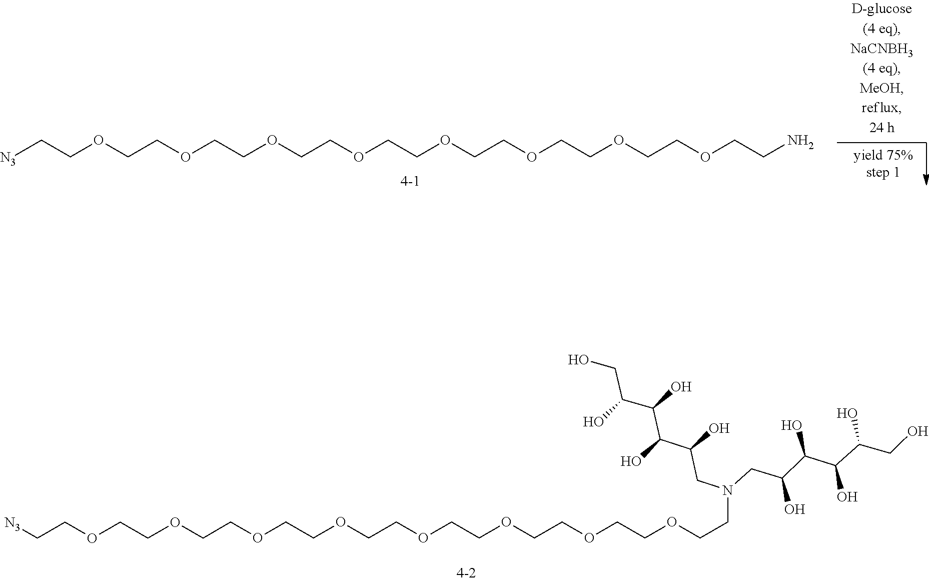 [SMILES: [N-]=[N+]=NCCOCCOCCOCCOCCOCCOCCOCCOCCN ; [N-]=[N+]=NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]

To the solution of 4-1 (2.5 g, 5.701 mmol) in MeOH (20 mL) was added D-Glucose (4.11 g, 22.804 mmol) and NaBH3CN (1.385 mL, 22.804 mmol). The mixture was stirred at reflux for 24 h to complete. Then the resulting solution concentrated to dryness and the residue was purified by reverse phase chromatography (C8 column, eluting with 0-45% methanol in water with 0.01% TFA) to afford the product 4-2 as yellow oil. ESI m/z: 767.5 (M+H)⁺.

### Step 2

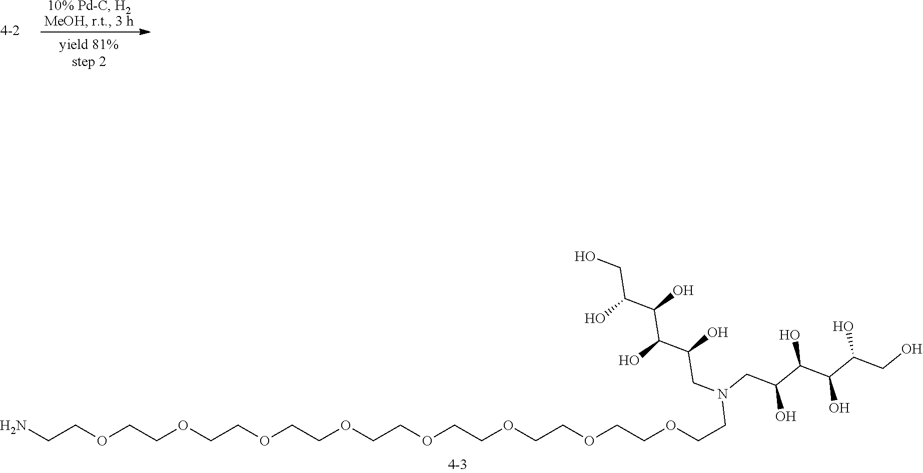 [SMILES: "10% Pd-C, H2MeOH, r.t., 3 h" ; NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]

To the solution of 4-2 (3.3 g, 4.303 mmol) in MeOH (20 mL) was added Pd/C (10% wt, 330 mg) under nitrogen and equipped with H₂ balloon. The reaction system was degassed and backfilled with hydrogen for three times and then stirred at room temperature under hydrogen atmosphere for 3 h to complete. The resulting mixture was filtered to remove catalyst solid and the filtrate was concentrated, then purified by reverse phase chromatography (C8 column, eluting with 0-25% acetonitrile in water with 0.01% TFA) to afford the product 4-3 (2.6 g, 3.510 mmol, 81.50%) as colorless oil. ESI m/z: 371.3 (M/2+H)⁺, 741.4 (M+H)⁺.

### Step 3

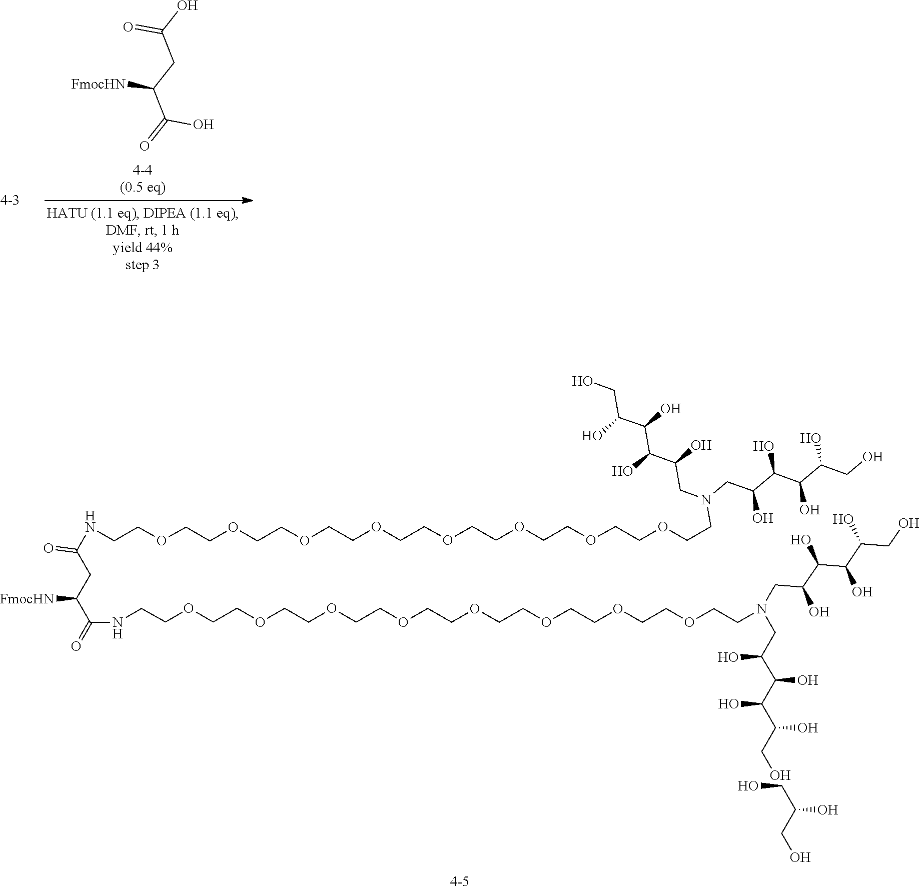

A solution of compound 4-4 (0.62 g, 1.755 mmol) in DMF (5 mL) was added HATU (1.47 g, 3.860 mmol) followed by DIPEA (0.50 g, 3.860 mmol). After stirring at room temperature for 15 min, the solution was added in dropwise manner into the solution of 4-3 (2.6 g, 3.510 mmol) in DMF (5 mL). After addition, the solution was stirred at room temperature for another 1 h to complete. The completed solution was then purified directly by reverse phase chromatography (C8 column, eluting with 0-40% acetonitrile in water with 0.01% TFA) to afford the product 4-5 (1.4 g, 0.777 mmol, 44.30%) as colorless oil. ESI m/z: 601.0 (M/3+H)⁺, 901.0 (M/2+H)⁺.

### Step 4

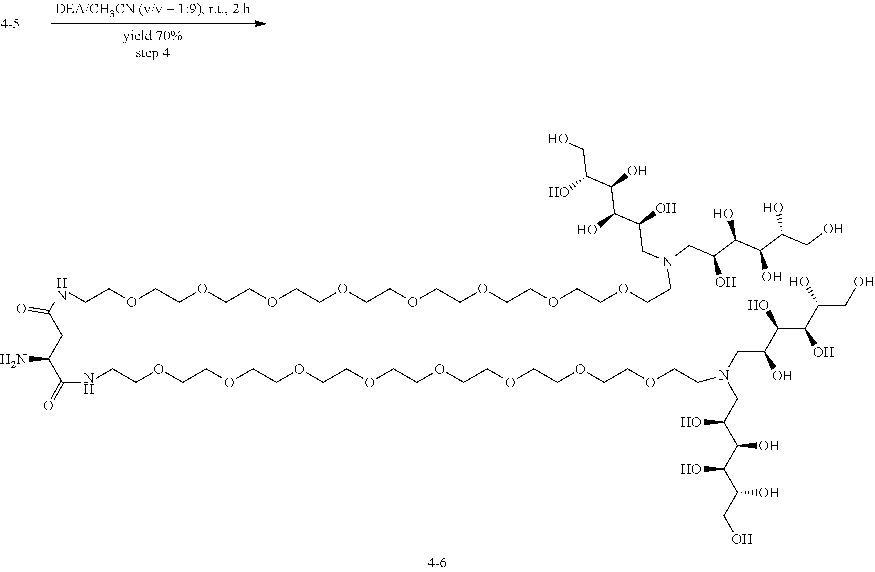 [SMILES: N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]

To the solution of 4-5 (1.4 g, 0.777 mmol) in MeCN (6 mL) was diethyl amine (0.7 mL, 8.930 mmol). The mixture was stirred at room temperature for 2 h to achieve complete deprotection. Then the resulting solution was concentrated under reduced pressure to remove most of diethyl amine, and the residue was purified by reverse phase chromatography (C8 column, eluting with 0-20% acetonitrile in water with 0.01% TFA) to get desired fractions, which was freeze-dried to afford 4-6 as sticky colorless oil. ESI m/z: 526.9 (M/3+H)⁺, 789.9 (M/2+H)⁺. 1HNMR (400 MHz, DMSO-d6) δ 8.36 (t, J=5.6 Hz, 1H), 8.24 (t, J=5.6 Hz, 1H), 5.88-4.41 (m, 15H), 3.99-3.79 (m, 4H), 3.61-3.56 (m, 12H), 3.52-3.49 (m, 60H), 3.49-3.40 (m, 12H), 3.27-3.19 (m, 6H), 3.04-2.86 (m, 16H), 2.65-2.06 (m, 2H), 1.15 (t, J=7.2 Hz, 2H) ppm.

### Example 5: Preparation of an Exemplary Polar Group

### Step 1

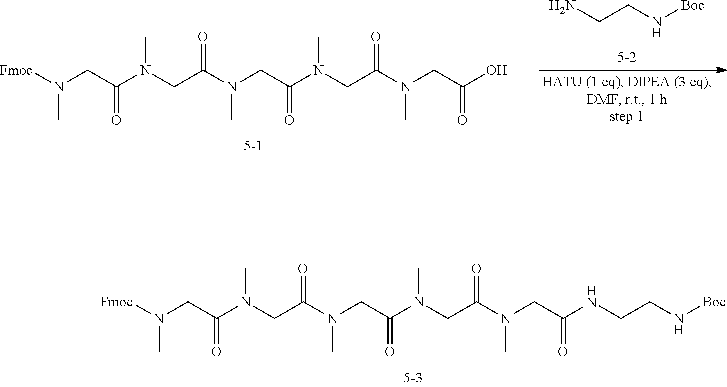

A solution of 5-1 (300 mg, 0.504 mmol), 5-2 (81 mg, 0.504 mmol) and HATU (192 mg, 0.504 mmol) in anhydrous DMF (10 mL) was stirred at room temperature for 5 min, then DIPEA (196 mg, 1.512 mmol) was added. The resulting solution was stirred for another 1 hr at r.t. until LCMS indicated complete reaction. The reaction solution was purified directly by reverse phase liquid chromatography to give 5-3 (227 mg, 0.308 mmol, 61.19%) as a white solid.

### Step 2

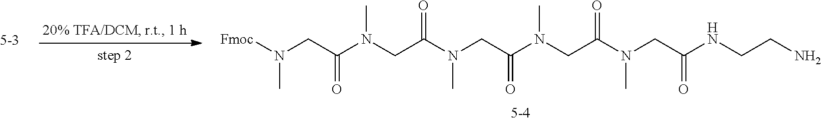

A solution of 5-3 (227 mg, 0.308 mmol) and TFA (2 mL) in anhydrous DCM (8 mL) was stirred at room temperature for 1 hr until LCMS indicated all starting amine was disappeared and desired product was detected. Then the solution was concentrated to dryness to 5-4 (185 mg, 0.290 mmol, 94.18%) as yellow oil, used as such in the next step.

### Step 3

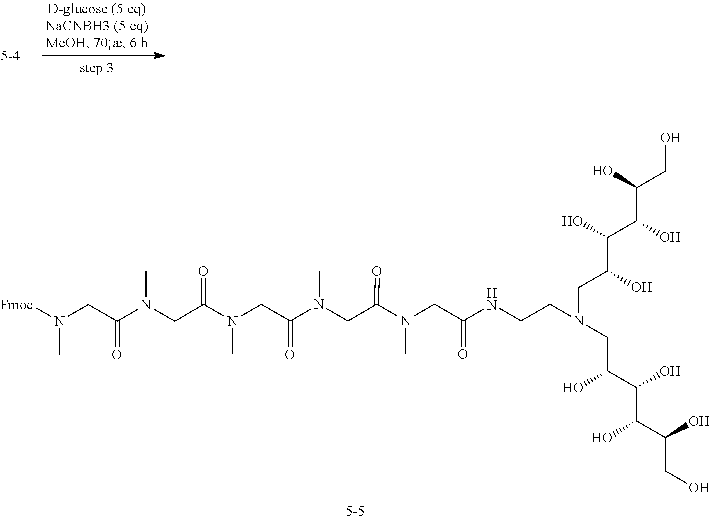

A solution of 5-4 (185 mg, 0.290 mmol) and D-Glucose (261 mg, 1.450 mmol) in anhydrous Methanol (50 mL) was heated at 50° C. for 30 min, then NaCNBH₃ (92 mg, 1.450 mmol) was added. The resulting solution was stirred for another 6 hr at 70° C. until indicated all starting amine was disappeared and the mass of desired product was detected. Then the reaction solution was concentrated and purified by reverse phase liquid chromatography to give 5-5 (125 mg, 0.129 mmol, 44.48%) as a white solid.

### Step 4

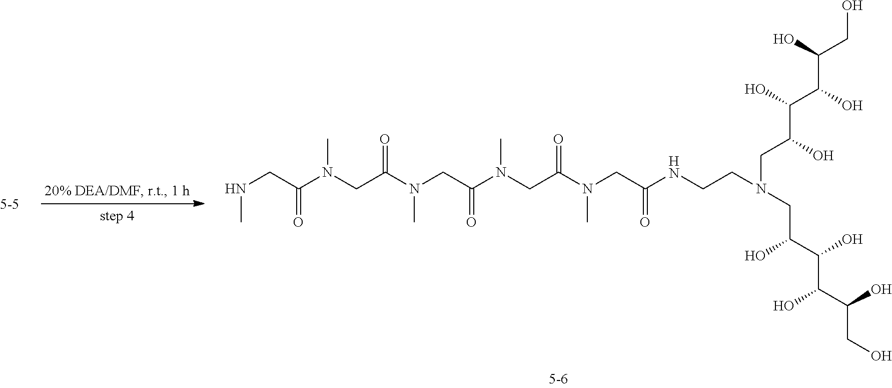 [SMILES: CNCC(=O)N(C)CC(=O)N(C)CC(=O)N(C)CC(=O)N(C)CC(=O)NCCN(C[C@@H](O)[C@H](O)[C@@H](O)[C@@H](O)CO)C[C@@H](O)[C@H](O)[C@@H](O)[C@@H](O)CO]

A solution of 5-5 (125 mg, 0.129 mmol) and DEA (1 mL) in anhydrous DMF (4 mL) was stirred at room temperature for 1 hr until LCMS indicated all starting amine was disappeared and desired product was detected. Then the solution was concentrated to dryness to 5-6 (91 mg, 0.122 mmol, 94.57%) as colorless oil, used as such in the next step.

### Example 6: Preparation of an Exemplary Polar Group

### Step 1

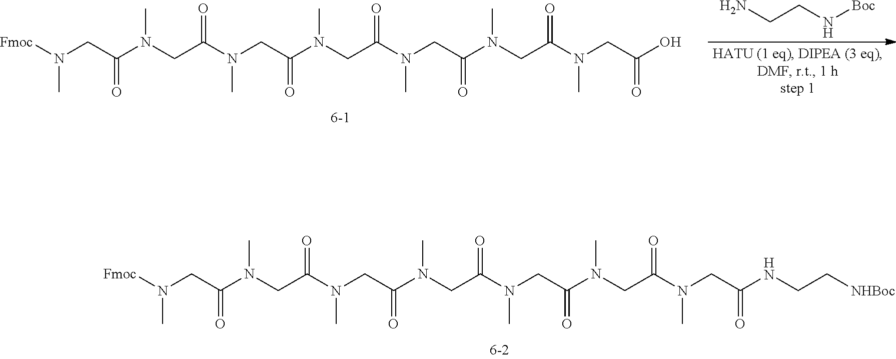

A solution of 6-1 (300 mg, 0.407 mmol), tert-butyl (2-aminoethyl)carbamate (65 mg, 0.407 mmol) and HATU (155 mg, 0.407 mmol) in anhydrous DMF (10 mL) was stirred at room temperature for 5 min, then DIPEA (158 mg, 1.221 mmol) was added. The resulting solution was stirred for another 1 hr at r.t. until LCMS indicated complete reaction. The reaction solution was purified directly by reverse phase liquid chromatography to give 6-2 (243 mg, 0.276 mmol, 67.81%) as a white solid.

### Step 2

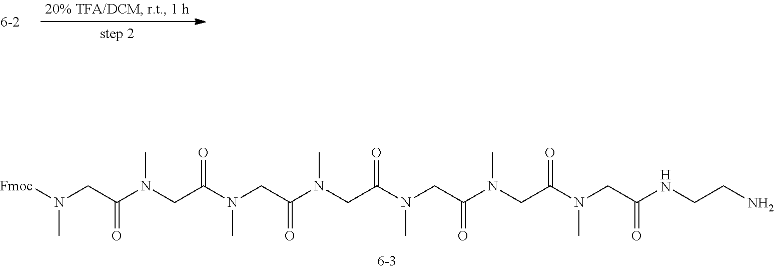

A solution of 6-2 (243 mg, 0.276 mmol) and TFA (2 mL) in anhydrous DCM (8 mL) was stirred at room temperature for 1 hr until LCMS indicated all starting amine was disappeared and desired product was detected. Then the solution was concentrated to dryness to 6-3 (207 mg, 0.265 mmol, 96.17%) as yellow oil, used as such in the next step.

### Step 3

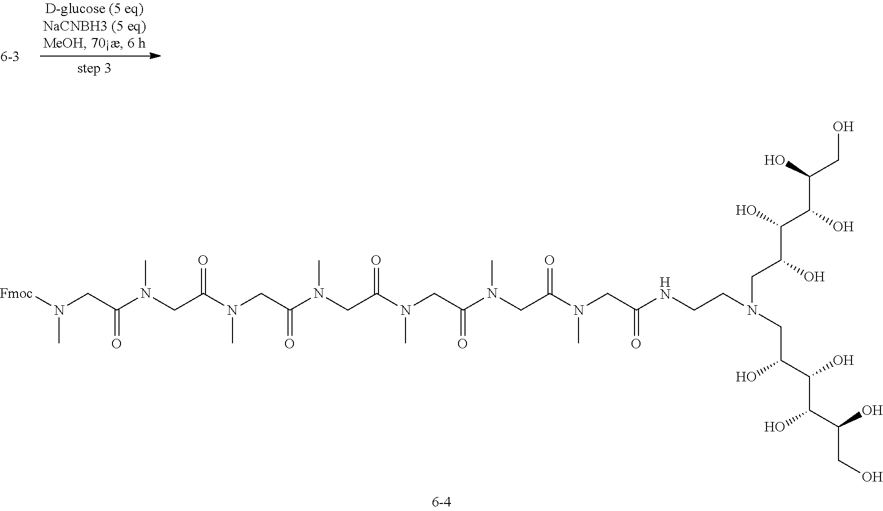

A solution of 6-3 (207 mg, 0.265 mmol) and D-Glucose (239 mg, 1.325 mmol) in anhydrous Methanol (50 mL) was heated at 50° C. for 30 min, then NaCNBH₃ (83 mg, 1.325 mmol) was added. The resulting solution was stirred for another 6 hr at 70° C. until indicated all starting amine was disappeared and the mass of desired product was detected. Then the reaction solution was concentrated and purified by reverse phase liquid chromatography to give 6-4 (147 mg, 0.133 mmol, 50.19%) as a white solid.

### Step 4

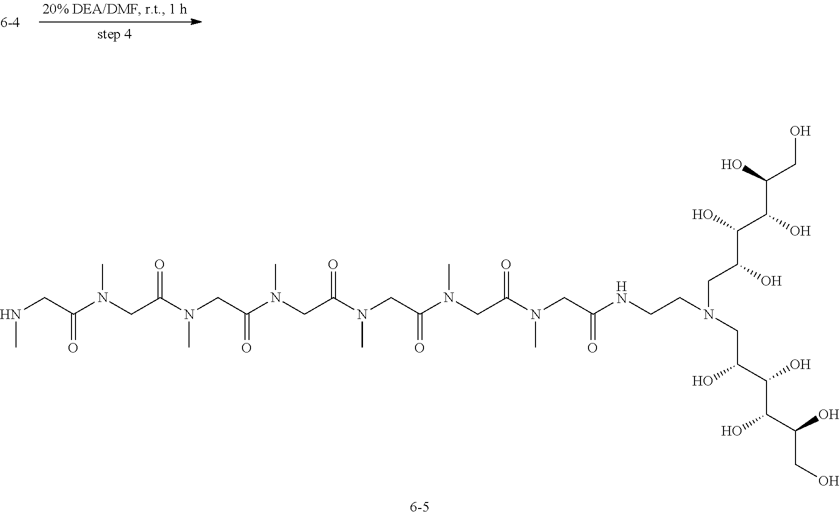 [SMILES: CNCC(=O)N(C)CC(=O)N(C)CC(=O)N(C)CC(=O)N(C)CC(=O)N(C)CC(=O)N(C)CC(=O)NCCN(C[C@@H](O)[C@H](O)[C@@H](O)[C@@H](O)CO)C[C@@H](O)[C@H](O)[C@@H](O)[C@@H](O)CO]

A solution of 6-4 (147 mg, 0.133 mmol) and DEA (1 mL) in anhydrous DMF (4 mL) was stirred at room temperature for 1 hr until LCMS indicated all starting amine was disappeared and desired product was detected. Then the solution was concentrated to dryness to 6-5 (103 mg, 0.116 mmol, 87.41%) as colorless oil, used as such in the next step.

### Example 7: Preparation of an Exemplary Polar Group

### Step 1

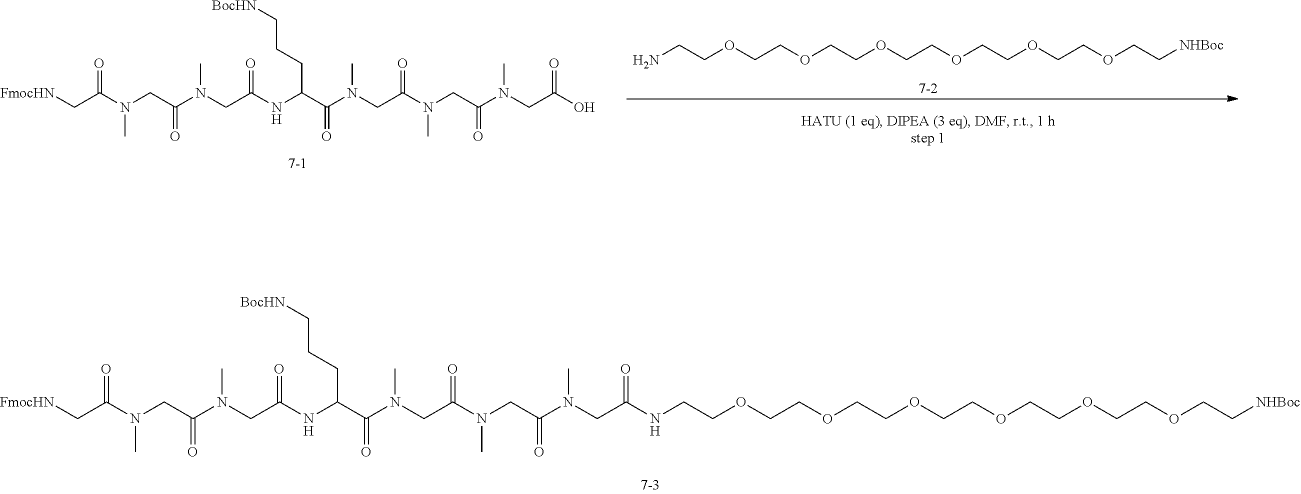

A solution of 7-1 (300 mg, 0.346 mmol), 7-2 (147 mg, 0.346 mmol) and HATU (132 mg, 0.346 mmol) in anhydrous DMF (5 mL) was stirred at room temperature for 5 min, then DIPEA (134 mg, 1.037 mmol) was added. The resulting solution was stirred for another 1 hr at r.t. until LCMS indicated complete reaction. The reaction solution was purified directly by reverse phase liquid chromatography to give 7-3 (310 mg, 0.243 mmol, 70.23%) as a white solid. Purity, 95%.

### Step 2

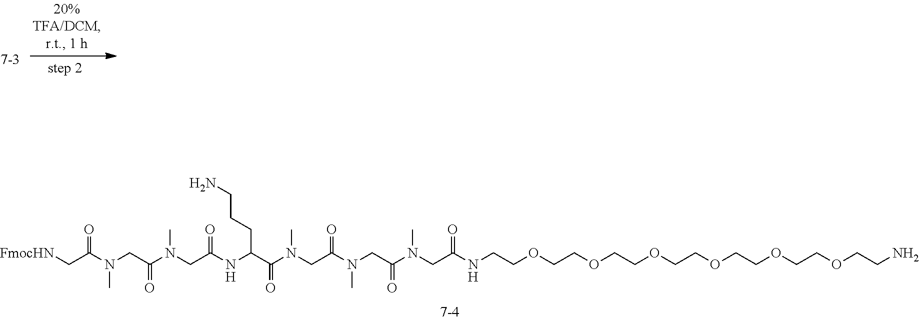

A solution of 7-3 (310 mg, 0.243 mmol) and TFA (1 mL) in anhydrous DCM (4 mL) was stirred at room temperature for 1 hr until LCMS indicated all starting amine was disappeared and desired product was detected. Then the solution was concentrated to dryness to 7-4 (255 mg, 0.238 mmol, 97.78%) as yellow oil, used as such in the next step. Purity, 95%.

### Step 3

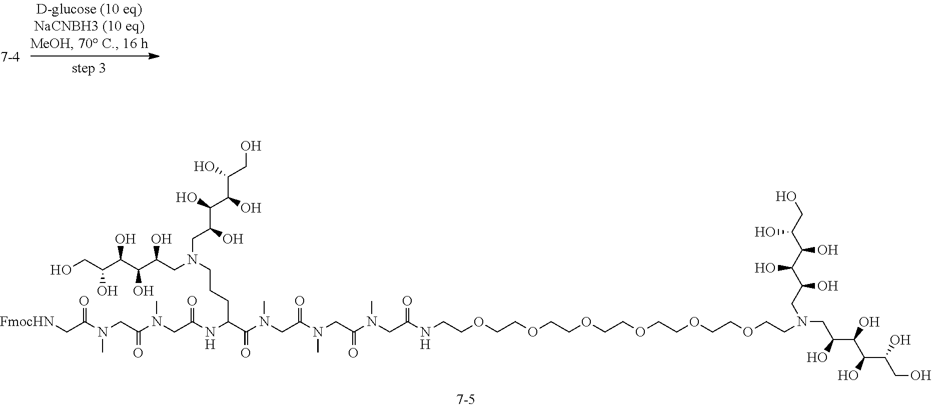

A solution of 7-4 (255 mg, 0.238 mmol) and D-Glucose (428 mg, 2.376 mmol) in anhydrous Methanol (50 mL) was heated at 50° C. for 30 min, then NaCNBH₃ (150 mg, 2.387 mmol) was added. The resulting solution was stirred for another 16 hr at 70° C. until indicated all starting amine was disappeared and the mass of desired product was detected. Then the reaction solution was concentrated and purified by reverse phase liquid chromatography to give 7-5 (185 mg, 0.107 mmol, 44.96%) as a white solid. Purity, 95%.

### Step 4

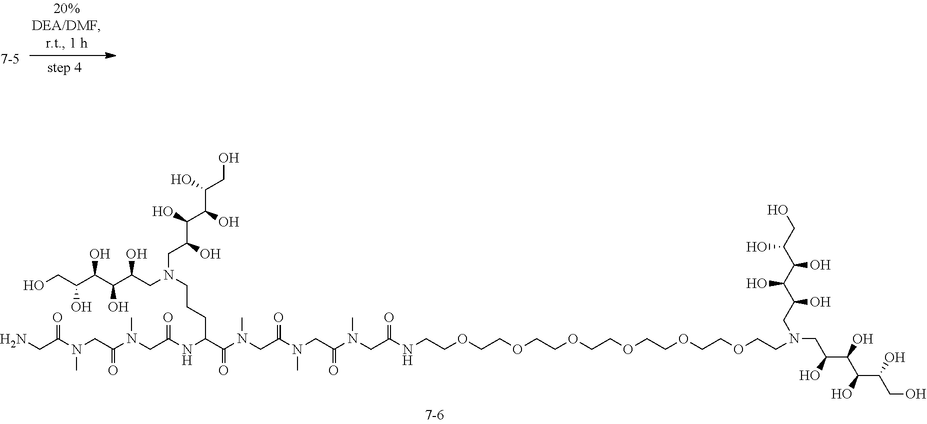 [SMILES: CN(CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)C(CCCN(C[C@H](O)[C@@H](O)[C@H](O)[C@H](O)CO)C[C@H](O)[C@@H](O)[C@H](O)[C@H](O)CO)NC(=O)CN(C)C(=O)CN(C)C(=O)CN]

A solution of 7-5 (185 mg, 0.107 mmol) and DEA (1 mL) in anhydrous DMF (4 mL) was stirred at room temperature for 1 hr until LCMS indicated all starting amine was disappeared and desired product was detected. Then the solution was concentrated to dryness to 7-6 (155 mg, 0.103 mmol, 96.08%) as colorless oil, used as such in the next step. Purity, 95%.

### Example 8: Preparation of an Exemplary Polar Group

Step 1

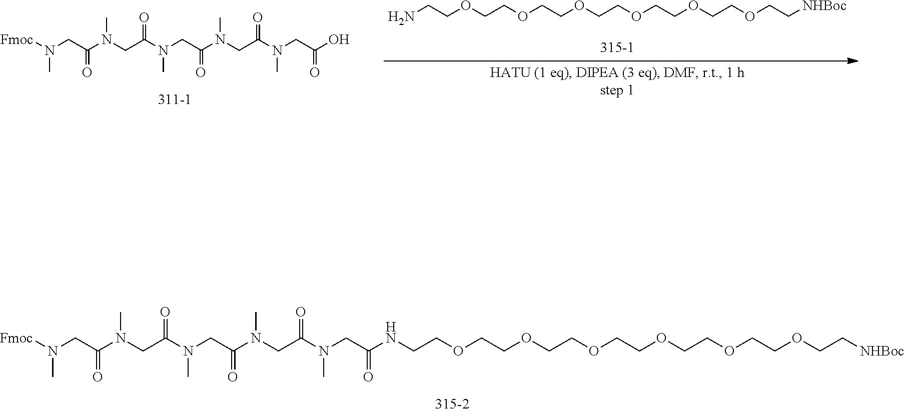

A solution of 311-1 (300 mg, 0.504 mmol), 315-1 (213 mg, 0.504 mmol) and HATU (192 mg, 0.407 mmol) in anhydrous DMF (10 mL) was stirred at room temperature for 5 min, then DIPEA (196 mg, 1.517 mmol) was added. The resulting solution was stirred for another 1 hr at r.t. until LCMS indicated complete reaction. The reaction solution was purified directly by reverse phase liquid chromatography to give 315-2 (315 mg, 0.314 mmol, 62.30%) as a white solid.

### Step 2

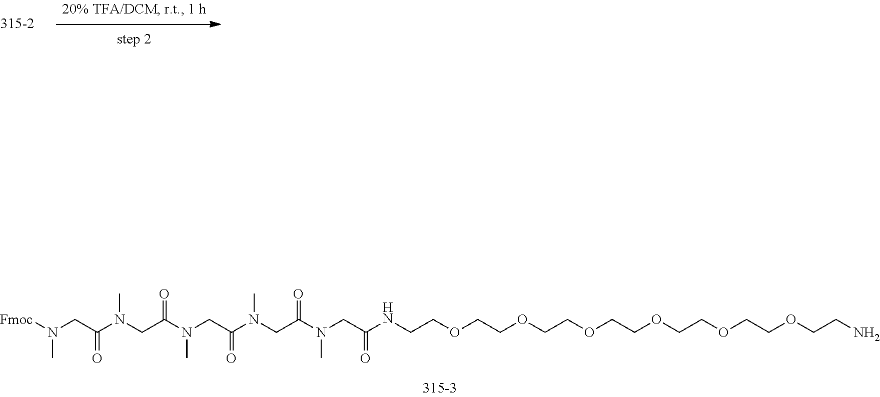

A solution of 315-2 (315 mg, 0.314 mmol) and TFA (2 mL) in anhydrous DCM (8 mL) was stirred at room temperature for 1 hr until LCMS indicated all starting amine was disappeared and desired product was detected. Then the solution was concentrated to dryness to 315-3 (264 mg, 0.293 mmol, 93.31%) as yellow oil, used as such in the next step.

### Step 3

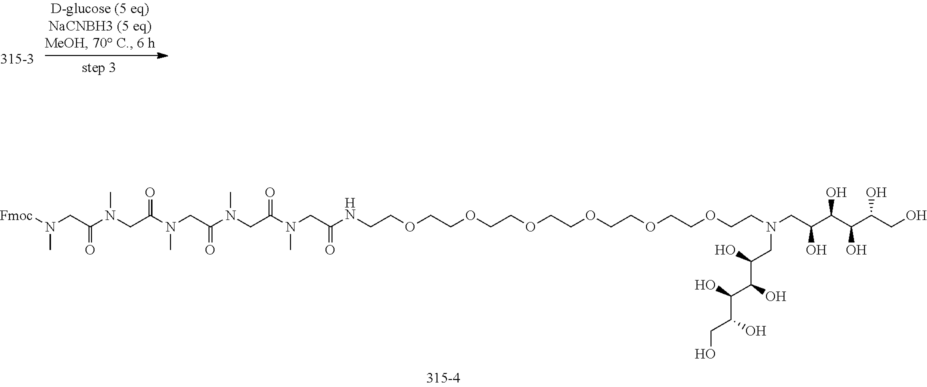

A solution of 315-3 (264 mg, 0.293 mmol) and D-Glucose (263 mg, 1.461 mmol) in anhydrous Methanol (50 mL) was heated at 50° C. for 30 min, then NaCNBH₃ (92 mg, 1.464 mmol) was added. The resulting solution was stirred for another 6 hr at 70° C. until indicated all starting amine was disappeared and the mass of desired product was detected. Then the reaction solution was concentrated and purified by reverse phase liquid chromatography to give 315-4 (233 mg, 0.189 mmol, 64.51%) as a white solid.

### Step 4

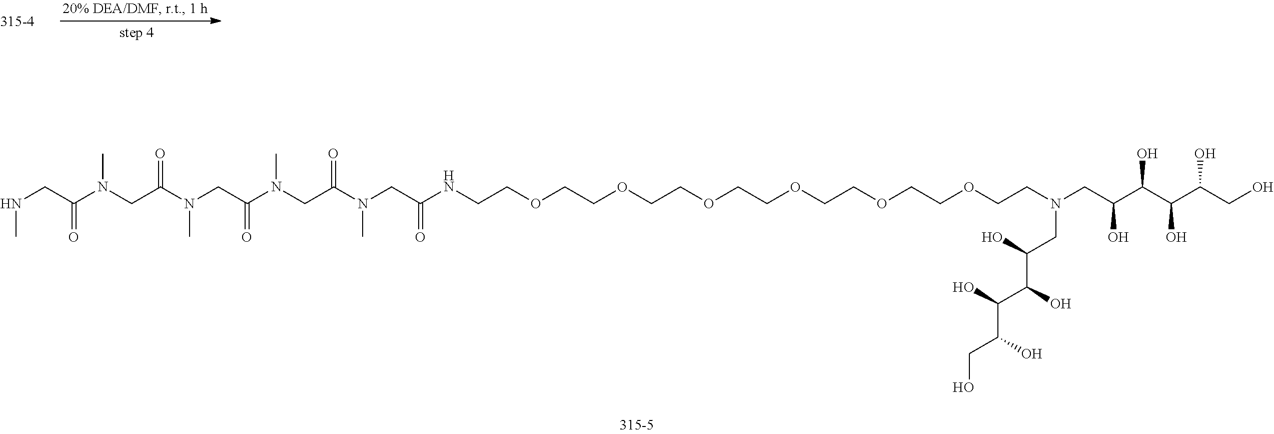 [SMILES: CNCC(=O)N(C)CC(=O)N(C)CC(=O)N(C)CC(=O)N(C)CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 315-4 (233 mg, 0.189 mmol) and DEA (1 mL) in anhydrous DMF (4 mL) was stirred at room temperature for 1 hr until LCMS indicated all starting amine was disappeared and desired product was detected. Then the solution was concentrated to dryness to 315-5 (177 mg, 0.176 mmol, 93.12%) as colorless oil, used as such in the next step.

### Example 9: Preparation of an Exemplary Polar Group

### Step 1

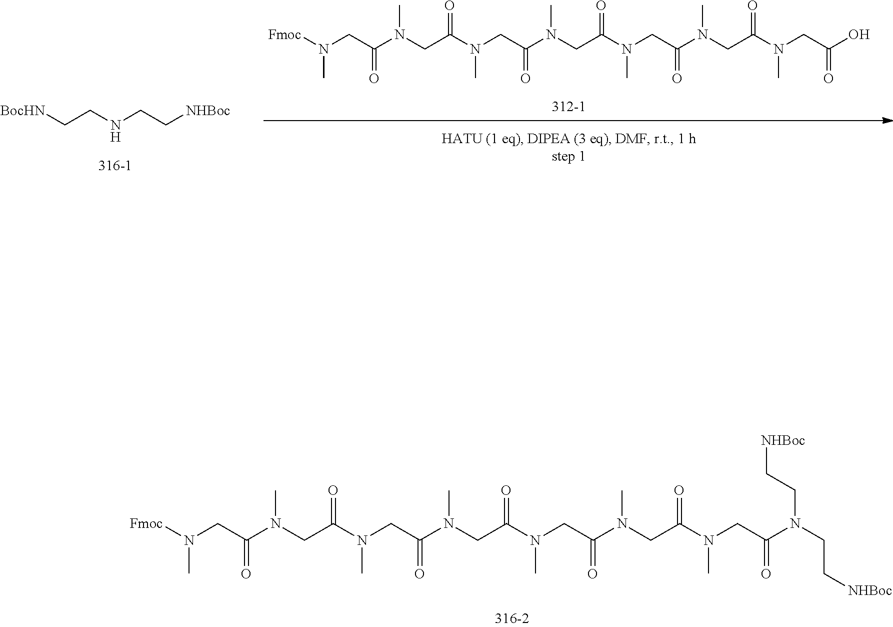

A solution of 316-1 (82 mg, 0.270 mmol), 312-1 (200 mg, 0.270 mmol) and HATU (103 mg, 0.270 mmol) in anhydrous DMF (10 mL) was stirred at room temperature for 5 min, then DIPEA (105 mg, 0.812 mmol) was added. The resulting solution was stirred for another 1 hr at r.t. until LCMS indicated complete reaction. The reaction solution was purified directly by reverse phase liquid chromatography to give 316-2 (205 mg, 0.200 mmol, 74.07%) as a white solid.

### Step 2

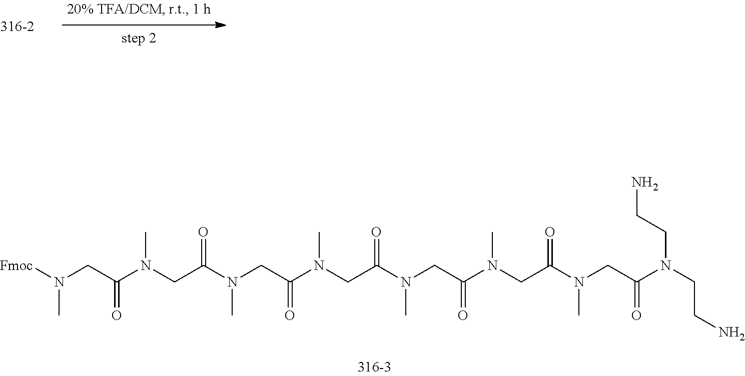

A solution of 316-2 (205 mg, 0.200 mmol) and TFA (1 mL) in anhydrous DCM (4 mL) was stirred at room temperature for 1 hr until LCMS indicated all starting amine was disappeared and desired product was detected. Then the solution was concentrated to dryness to 316-3 (157 mg, 0.191 mmol, 93.17%) as yellow oil, used as such in the next step.

### Step 3

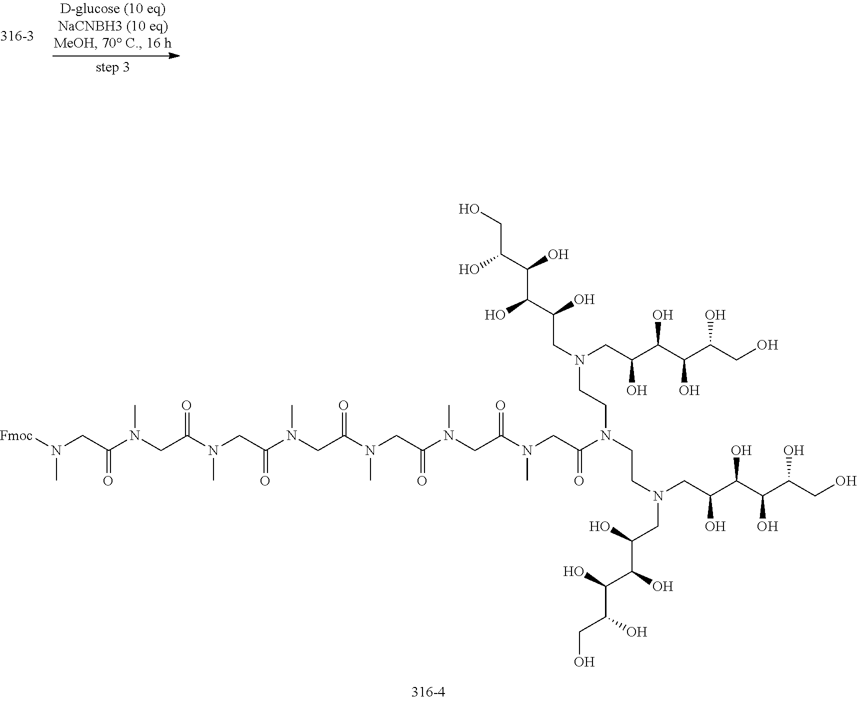

A solution of 316-3 (157 mg, 0.191 mmol) and D-Glucose (172 mg, 0.955 mmol) in anhydrous Methanol (50 mL) was heated at 50° C. for 30 min, then NaCNBH₃ (60 mg, 0.955 mmol) was added. The resulting solution was stirred for another 16 hr at 70° C. until indicated all starting amine was disappeared and the mass of desired product was detected. Then the reaction solution was concentrated and purified by reverse phase liquid chromatography to give 316-4 (135 mg, 0.091 mmol, 47.77%) as a white solid.

### Step 4

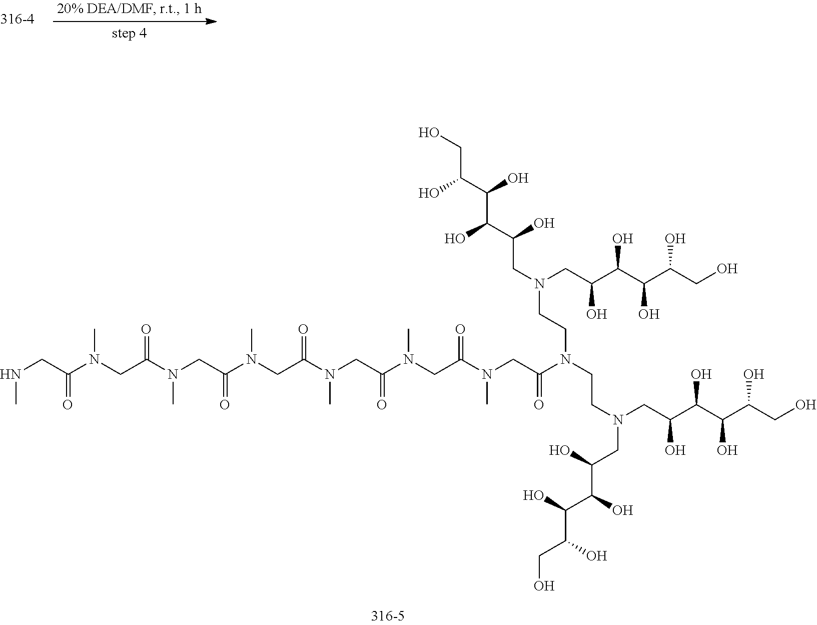 [SMILES: CNCC(=O)N(C)CC(=O)N(C)CC(=O)N(C)CC(=O)N(C)CC(=O)N(C)CC(=O)N(C)CC(=O)N(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO]

A solution of 316-4 (135 mg, 0.091 mmol) and DEA (1 mL) in anhydrous DMF (4 mL) was stirred at room temperature for 1 hr until LCMS indicated all starting amine was disappeared and desired product was detected. Then the solution was concentrated to dryness to 316-5 (114 mg, 0.090 mmol, 98.90%) as colorless oil, used as such in the next step.

### Example 10: Preparation of an Exemplary Polar Group

### Step 1

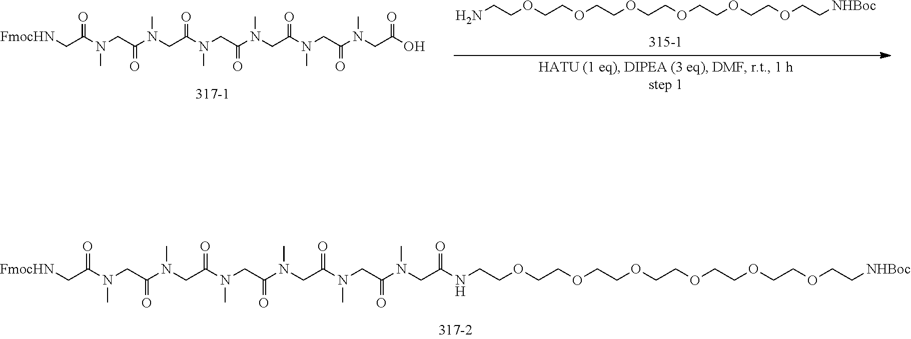

A solution of 317-1 (400 mg, 0.553 mmol), 315-1 (235 mg, 0.554 mmol) and HATU (210 mg, 0.553 mmol) in anhydrous DMF (6 mL) was stirred at room temperature for 5 min, then DIPEA (214 mg, 1.656 mmol) was added. The resulting solution was stirred for another 1 hr at room temperature until LCMS indicated complete reaction. The reaction solution was purified directly by reverse phase liquid chromatography to give 317-2 (410 mg, 0.363 mmol, 65.59%) as a white solid.

### Step 2

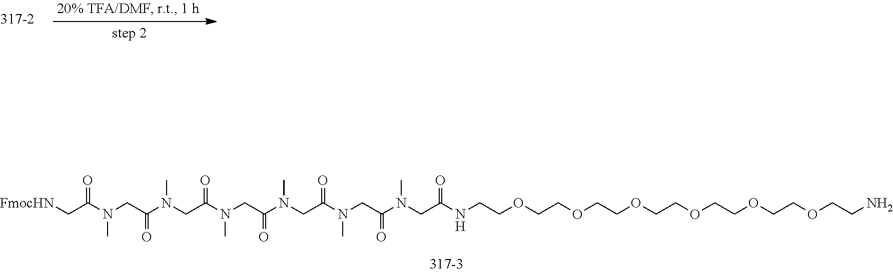

A solution of 317-2 (410 mg, 0.363 mmol) and TFA (2 mL) in anhydrous DCM (8 mL) was stirred at room temperature for 1 hr until LCMS indicated all starting amine was disappeared and desired product was detected. Then the solution was concentrated to dryness to 317-3 (365 mg, 0.354 mmol, 97.60%) as yellow oil, used as such in the next step.

### Step 3

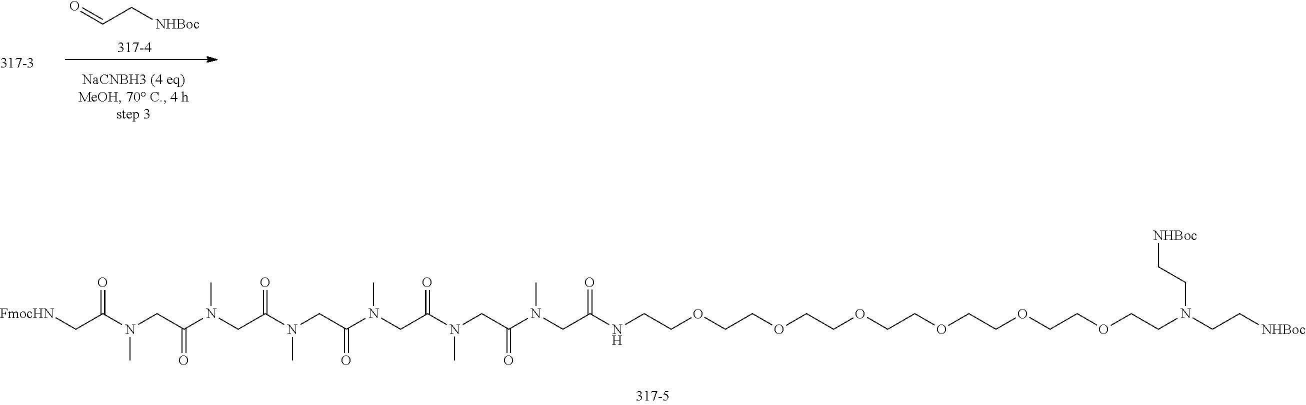

A solution of 317-3 (365 mg, 0.354 mmol) and 317-4 (338 mg, 2.123 mmol) in anhydrous Methanol (50 mL) was heated at 50° C. for 30 min, then NaCNBH₃ (134 mg, 2.132 mmol) was added. The resulting solution was stirred for another 4 hr at 70° C. until indicated all starting amine was disappeared and the mass of desired product was detected. Then the reaction solution was concentrated and purified by reverse phase liquid chromatography to give 317-5 (310 mg, 0.235 mmol, 66.51%) as a white solid.

### Step 4

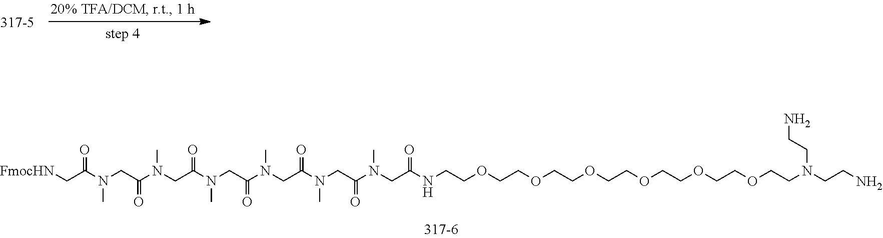

A solution of 317-5 (310 mg, 0.235 mmol) and TFA (2 mL) in anhydrous DCM (8 mL) was stirred at room temperature for 1 hr until LCMS indicated all starting amine was disappeared and desired product was detected. Then the solution was concentrated to dryness to 317-6 (250 mg, 0.224 mmol, 95.30%) as yellow oil, used as such in the next step.

### Step 5

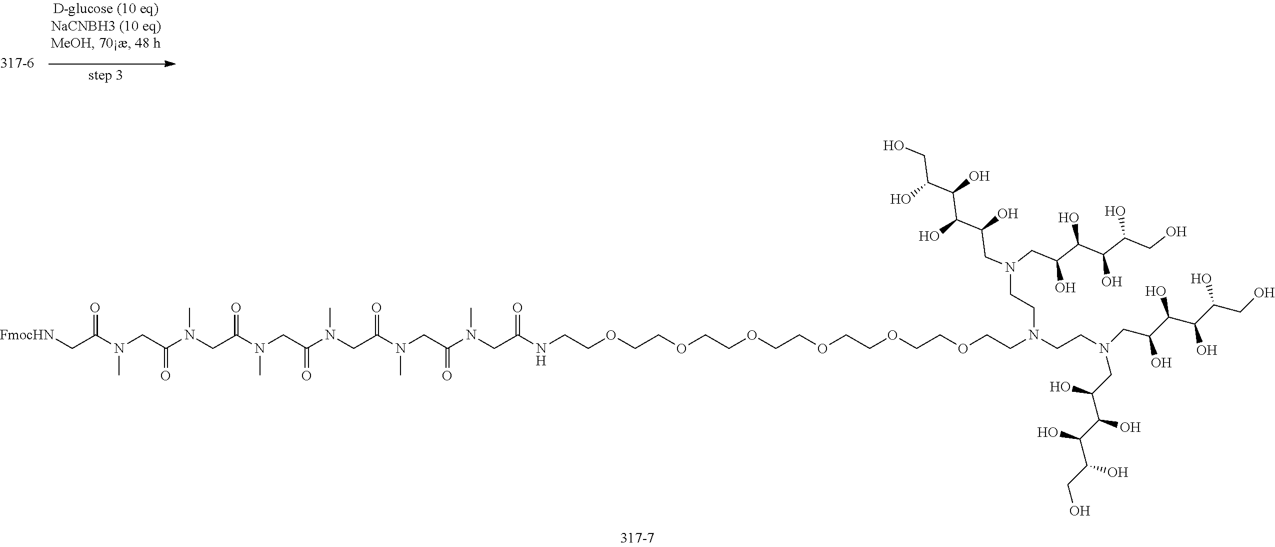

A solution of 317-6 (250 mg, 0.224 mmol) and D-Glucose (484 mg, 2.687 mmol) in anhydrous Methanol (50 mL) was heated at 50° C. for 30 min, then NaCNBH₃ (169 mg, 2.689 mmol) was added. The resulting solution was stirred for another 48 hr at 70° C. until indicated all starting amine was disappeared and the mass of desired product was detected. Then the reaction solution was concentrated and purified by reverse phase liquid chromatography to give 317-7 (174 mg, 0.098 mmol, 43.81%) as a white solid.

### Step 6

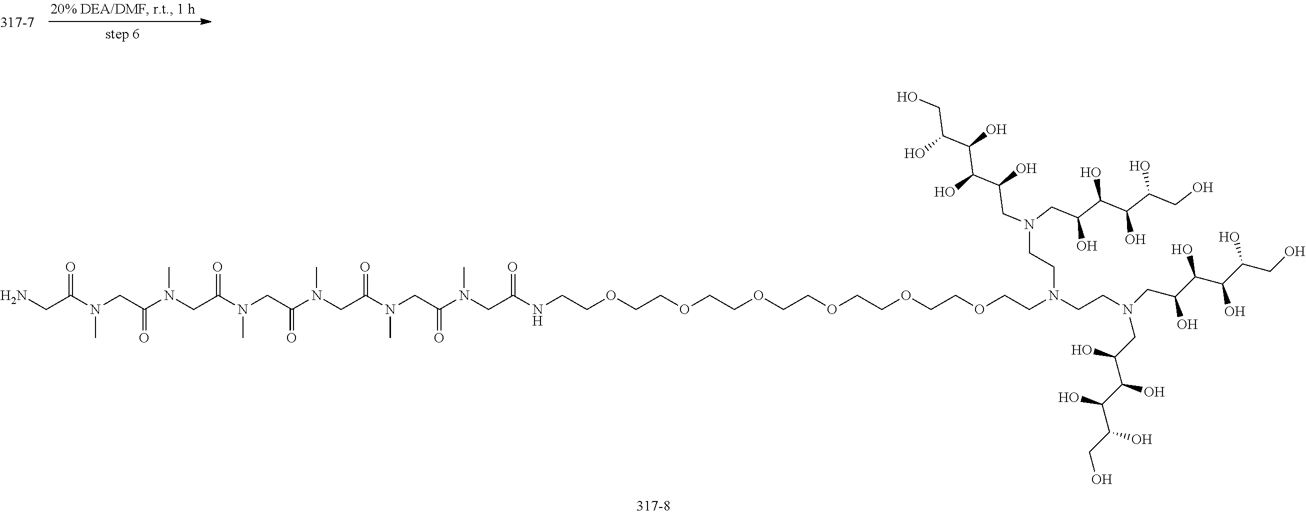 [SMILES: CN(CC(=O)NCCOCCOCCOCCOCCOCCOCCN(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)CN(C)C(=O)CN(C)C(=O)CN(C)C(=O)CN]

A solution of 317-7 (174 mg, 0.098 mmol) and DEA (1 mL) in anhydrous DMF (4 mL) was stirred at room temperature for 1 hr until LCMS indicated all starting amine was disappeared and desired product was detected. Then the solution was concentrated to dryness to 317-8 (145 mg, 0.094 mmol, 95.41%) as colorless oil, used as such in the next step.

### Example 11: Preparation of an Exemplary Polar Group

### Step 1

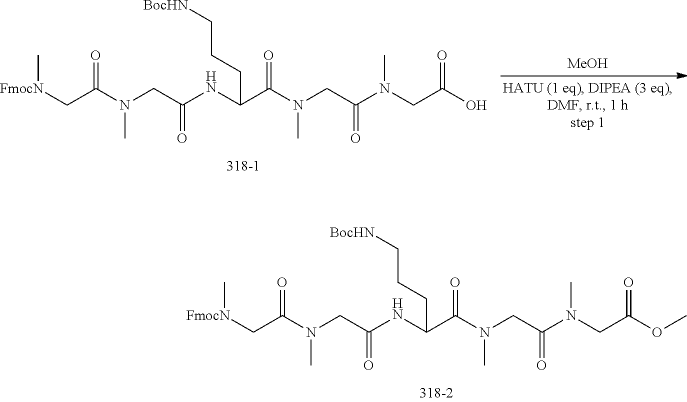

A solution of 318-1 (200 mg, 0.271 mmol), MeOH (43 mg, 1.342 mmol) and HATU (103 mg, 0.271 mmol) in anhydrous DMF (10 mL) was stirred at room temperature for 5 min, then DIPEA (105 mg, 0.812 mmol) was added. The resulting solution was stirred for another 1 hr at r.t. until LCMS indicated complete reaction. The reaction solution was purified directly by reverse phase liquid chromatography to give 318-2 (167 mg, 0.222 mmol, 81.92%) as a white solid.

### Step 2

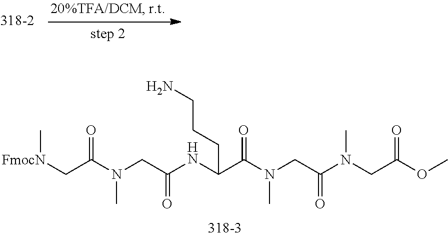

A solution of 318-2 (167 mg, 0.222 mmol) and TFA (1 mL) in anhydrous DCM (4 mL) was stirred at room temperature for 1 hr until LCMS indicated all starting amine was disappeared and desired product was detected. Then the solution was concentrated to dryness to 318-3 (135 mg, 0.207 mmol, 93.24%) as yellow oil, used as such in the next step.

### Step 3

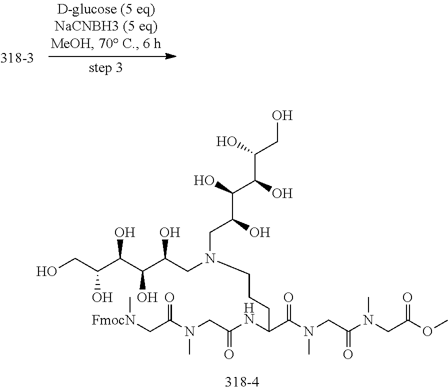

A solution of 318-3 (135 mg, 0.207 mmol) and D-Glucose (186 mg, 1.032 mmol) in anhydrous Methanol (50 mL) was heated at 50° C. for 30 min, then NaCNBH₃ (65 mg, 1.034 mmol) was added. The resulting solution was stirred for another 6 hr at 70° C. until indicated all starting amine was disappeared and the mass of desired product was detected. Then the reaction solution was concentrated and purified by reverse phase liquid chromatography to give 318-4 (121 mg, 0.123 mmol, 59.42%) as a white solid.

### Step 4

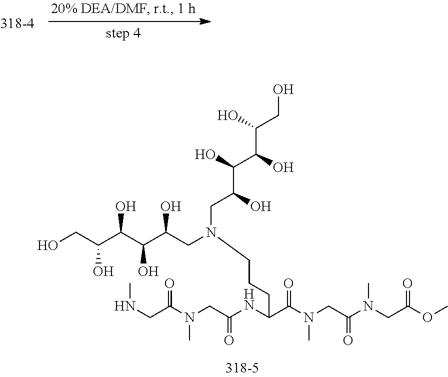 [SMILES: CNCC(=O)N(C)CC(=O)NC(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)OC]

A solution of 318-4 (121 mg, 0.123 mmol) and DEA (1 mL) in anhydrous DMF (4 mL) was stirred at room temperature for 1 hr until LCMS indicated all starting amine was disappeared and desired product was detected. Then the solution was concentrated to dryness to 318-5 (75 mg, 0.099 mmol, 80.49%) as colorless oil, used as such in the next step.

### Example 12: Preparation of an Exemplary Polar Group

### Step 1

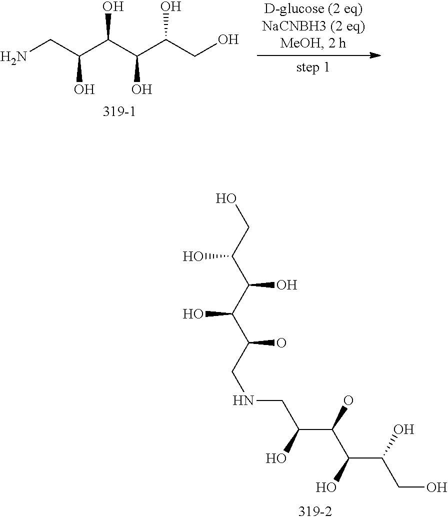

A solution of 319-1 (2.0 g, 0.011 mol) and D-Glucose (4.0 g, 0.022 mol) in anhydrous Methanol (50 mL) was heated at 50° C. for 30 min, then NaCNBH₃ (1.4 g, 0.022 mol) was added. The resulting solution was stirred for another 2 hr at 70° C. until indicated all starting amine was disappeared and the mass of desired product was detected. Then the reaction solution was concentrated and purified by reverse phase liquid chromatography to give 319-2 (2.6 g, 7.529 mmol, 68.44%) as a white solid.

### Step 2

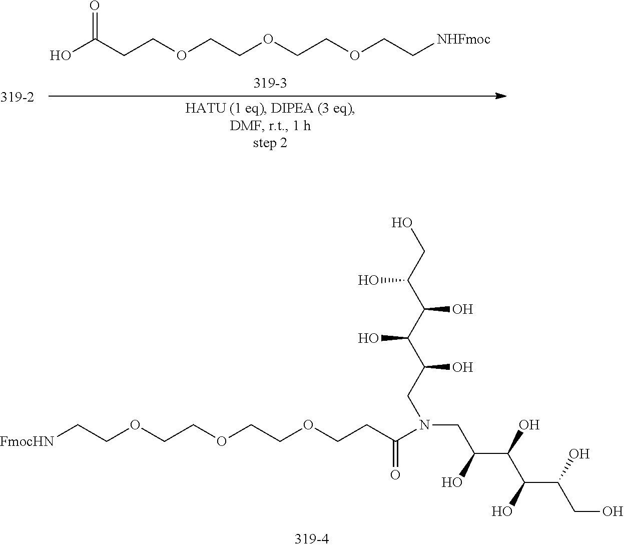

A solution of 319-2 (2.6 g, 7.529 mmol), 319-3 (3.3 g, 7.529 mmol) and HATU (2.9 g, 7.529 mmol) in anhydrous DMF (10 mL) was stirred at room temperature for 5 min, then DIPEA (2.9 g, 22.586 mmol) was added. The resulting solution was stirred for another 1 hr at r.t. until LCMS indicated complete reaction. The reaction solution was purified directly by reverse phase liquid chromatography to give 319-4 (1.5 g, 1.946 mmol, 25.85%) as a white solid.

### Step 3

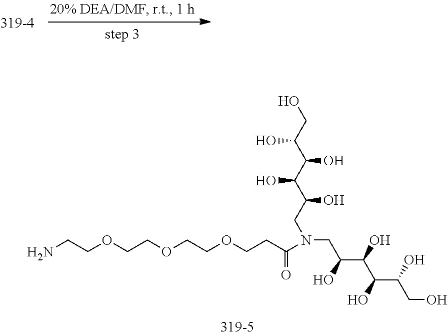 [SMILES: NCCOCCOCCOCCC(=O)N(C[C@H](O)[C@@H](O)[C@H](O)[C@H](O)CO)C[C@H](O)[C@@H](O)[C@H](O)[C@H](O)CO]

A solution of 319-4 (1.5 g, 1.946 mmol) and DEA (2 mL) in anhydrous DMF (8 mL) was stirred at room temperature for 1 hr until LCMS indicated all starting amine was disappeared and desired product was detected. Then the solution was concentrated to dryness to 319-5 (950 mg, 1.732 mmol, 89.00%) as colorless oil, used as such in the next step.

### Step 4

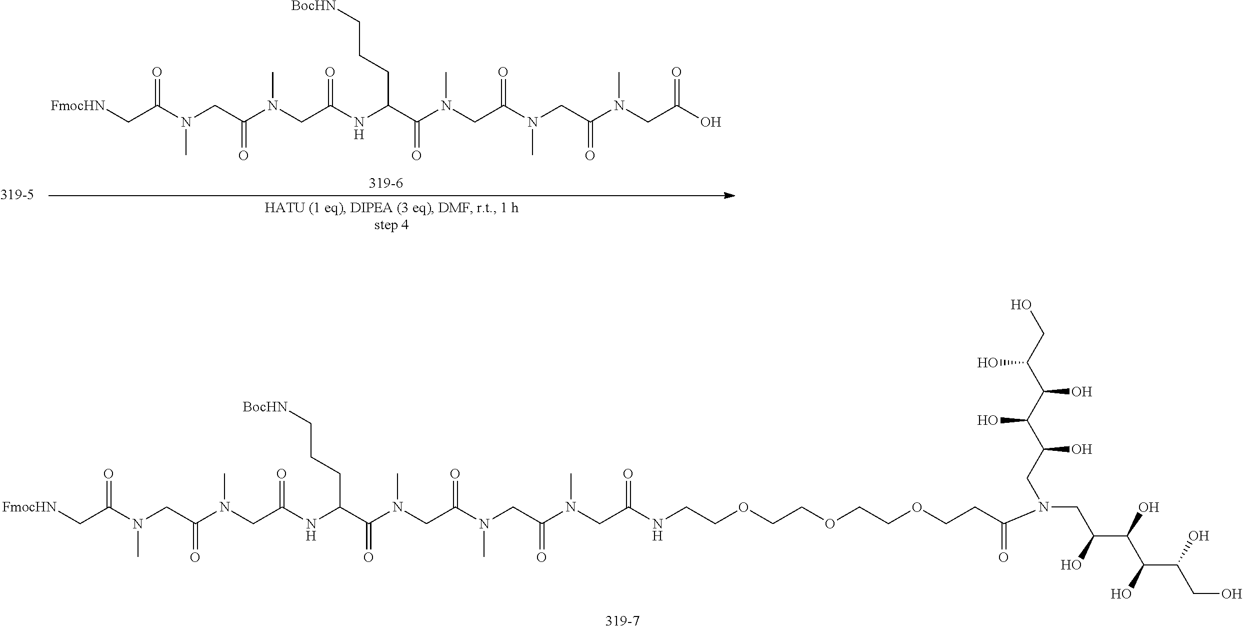

A solution of 319-5 (950 mg, 1.732 mmol), 319-6 (1501 mg, 1.732 mmol) and HATU (658 mg, 1.732 mmol) in anhydrous DMF (10 mL) was stirred at room temperature for 5 min, then DIPEA (670 mg, 5.184 mmol) was added. The resulting solution was stirred for another 1 hr at r.t. until LCMS indicated complete reaction. The reaction solution was purified directly by reverse phase liquid chromatography to give 319-7 (700 mg, 0.501 mmol, 28.93%) as a white solid.

### Step 5

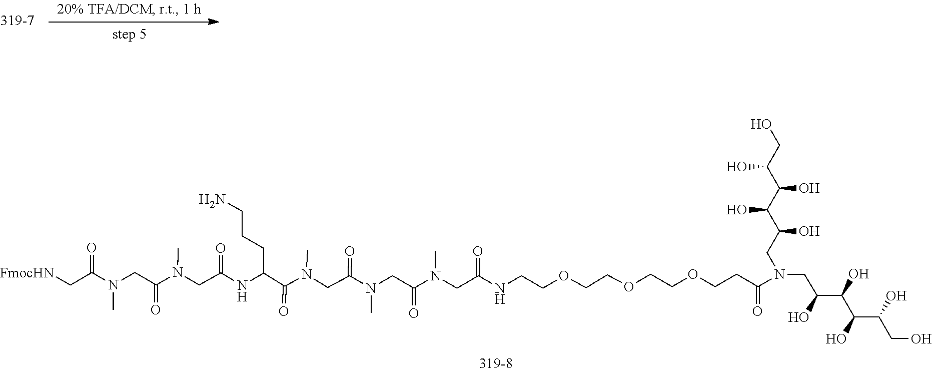

A solution of 319-7 (300 mg, 0.215 mmol) and TFA (1 mL) in anhydrous DCM (4 mL) was stirred at room temperature for 1 hr until LCMS indicated all starting amine was disappeared and desired product was detected. Then the solution was concentrated to dryness to 319-8 (245 mg, 0.189 mmol, 87.91%) as yellow oil, used as such in the next step.

### Step 6

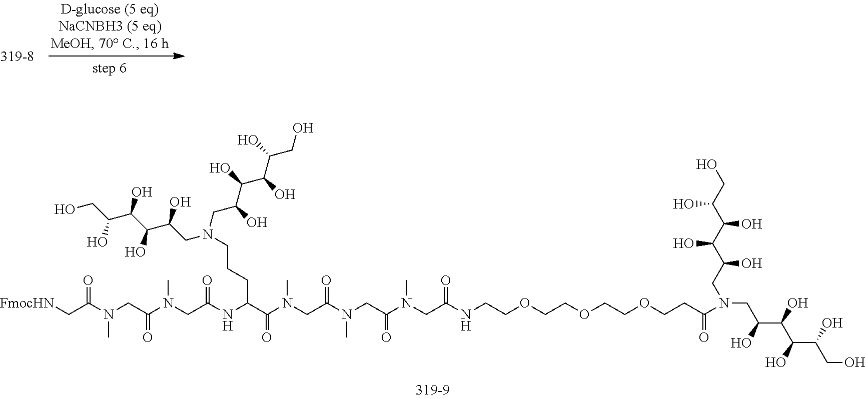

A solution of 319-8 (245 mg, 0.189 mmol) and D-Glucose (170 mg, 0.945 mmol) in anhydrous Methanol (50 mL) was heated at 50° C. for 30 min, then NaCNBH₃ (60 mg, 0.945 mmol) was added. The resulting solution was stirred for another 16 hr at 70° C. until indicated all starting amine was disappeared and the mass of desired product was detected. Then the reaction solution was concentrated and purified by reverse phase liquid chromatography to give 319-9 (140 mg, 0.086 mmol, 45.50%) as a white solid.

### Step 7

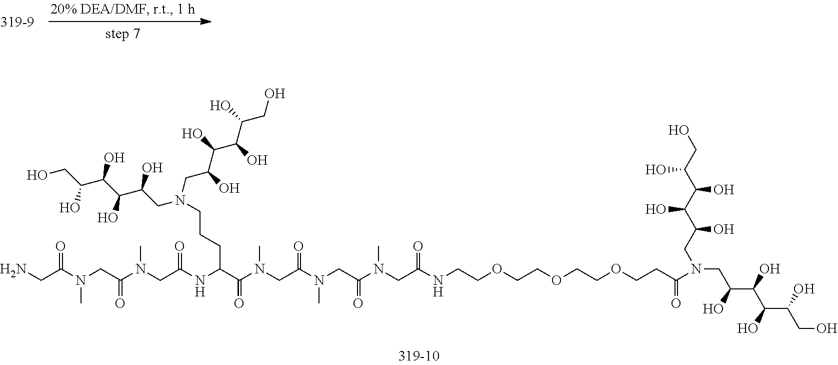 [SMILES: CN(CC(=O)NCCOCCOCCOCCC(=O)N(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)C(CCCN(C[C@H](O)[C@@H](O)[C@H](O)[C@H](O)CO)C[C@H](O)[C@@H](O)[C@H](O)[C@H](O)CO)NC(=O)CN(C)C(=O)CN(C)C(=O)CN]

A solution of 319-9 (140 mg, 0.086 mmol) and DEA (1 mL) in anhydrous DMF (4 mL) was stirred at room temperature for 1 hr until LCMS indicated all starting amine was disappeared and desired product was detected. Then the solution was concentrated to dryness to 319-10 (112 mg, 0.080 mmol, 93.02%) as colorless oil, used as such in the next step.

### Example 13: Preparation of an Exemplary Polar Group

### Step 1

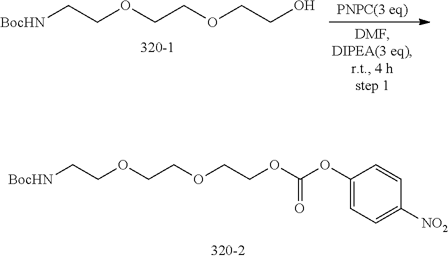 [SMILES: CC(C)(C)OC(=O)NCCOCCOCCO ; CC(C)(C)OC(=O)NCCOCCOCCOC(=O)Oc1ccc([N+](=O)[O-])cc1]

A solution of 320-1 (1.0 g, 4.011 mol) and DIPEA (1.6 g, 12.380 mol) in anhydrous DMF (15 mL) was stirred at room temperature for 5 min, then PNPC (3.7 g, 12.163 mmol) was added. The resulting solution was stirred for another 4 hr at r.t. until LCMS indicated all starting amine was disappeared and desired product was detected. Then the reaction solution was concentrated and purified by reverse phase liquid chromatography to give 320-2 (943 mg, 2.276 mmol, 56.73%) as a yellow solid.

### Step 2

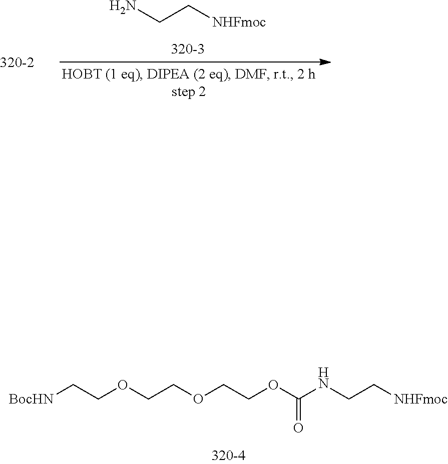

A solution of 320-2 (943 mg, 2.276 mmol), 320-3 (642 mg, 2.274 mmol) and HOBT (307 mg, 2.274 mmol) in anhydrous DMF (50 mL) was stirred at room temperature, then DIPEA (588 mg, 4.550 mmol) was added. The resulting solution was stirred for another 1 hr at r.t. until LCMS indicated all starting amine was disappeared and desired product was detected. The reaction solution was purified directly by reverse phase liquid chromatography to give 320-4 (850 mg, 1.524 mmol, 66.97%) as a white solid.

### Step 3

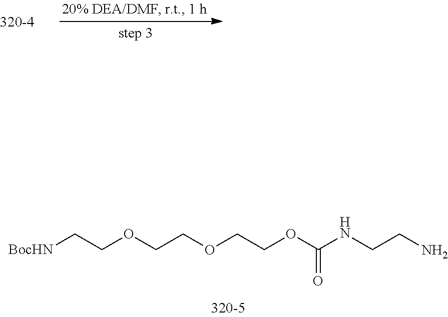 [SMILES: CC(C)(C)OC(=O)NCCOCCOCCOC(=O)NCCN]

A solution of 320-4 (850 mg, 1.524 mmol) and DEA (2 mL) in anhydrous DMF (8 mL) was stirred at room temperature for 1 hr until LCMS indicated all starting amine was disappeared and desired product was detected. Then the solution was concentrated to dryness to 320-5 (491 mg, 1.464 mmol, 96.06%) as colorless oil, used as such in the next step.

### Step 4

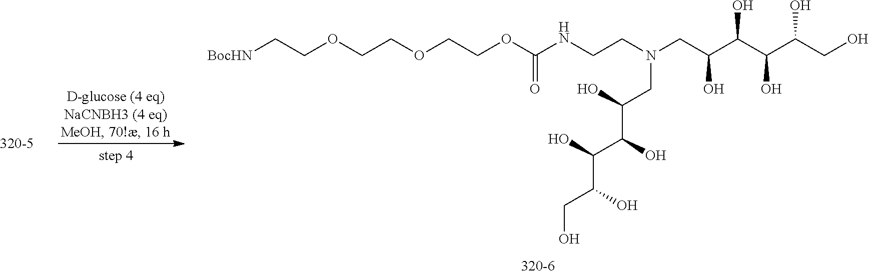 [SMILES: CC(C)(C)OC(=O)NCCOCCOCCOC(=O)NCCN(C[C@H](O)[C@@H](O)[C@H](O)[C@H](O)CO)C[C@H](O)[C@@H](O)[C@H](O)[C@H](O)CO]

A solution of 320-5 (491 mg, 1.464 mmol) and D-Glucose (1.05 g, 5.828 mmol) in anhydrous Methanol (50 mL) was heated at 50° C. for 30 min, then NaCNBH₃ (368 mg, 5.856 mmol) was added. The resulting solution was stirred for another 16 hr at 70° C. until indicated all starting amine was disappeared and the mass of desired product was detected. Then the reaction solution was concentrated and purified by reverse phase liquid chromatography to give 320-6 (470 mg, 0.708 mmol, 48.37%) as a white solid.

### Step 5

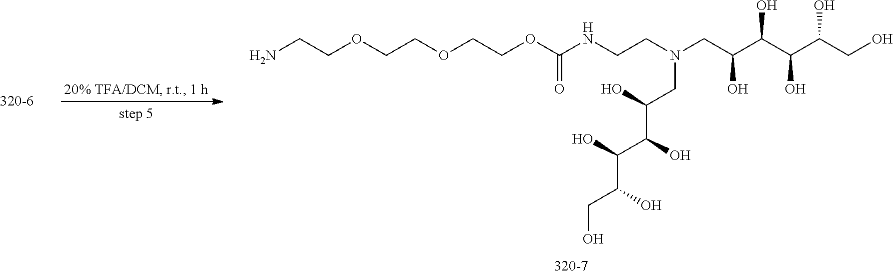 [SMILES: NCCOCCOCCOC(=O)NCCN(C[C@H](O)[C@@H](O)[C@H](O)[C@H](O)CO)C[C@H](O)[C@@H](O)[C@H](O)[C@H](O)CO]

A solution of 320-6 (470 mg, 0.708 mmol) and TFA (2 mL) in anhydrous DCM (8 mL) was stirred at room temperature for 1 hr until LCMS indicated all starting amine was disappeared and desired product was detected. Then the solution was concentrated to dryness to 320-7 (385 mg, 0.683 mmol, 96.48%) as yellow oil, used as such in the next step.

### Step 6

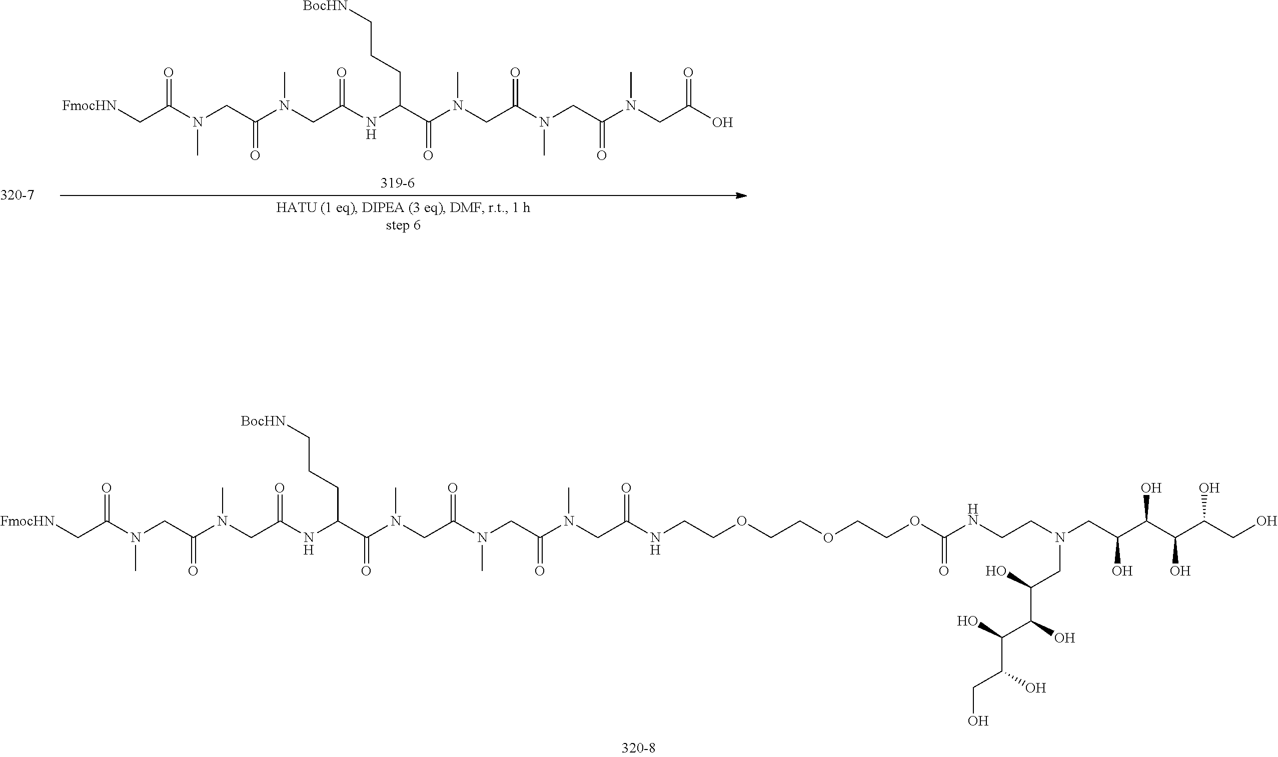

A solution of 320-7 (385 mg, 0.683 mmol), 319-6 (592 mg, 0.683 mmol) and HATU (260 mg, 0.684 mmol) in anhydrous DMF (6 mL) was stirred at room temperature for 5 min, then DIPEA (265 mg, 2.050 mmol) was added. The resulting solution was stirred for another 1 hr at r.t. until LCMS indicated complete reaction. The reaction solution was purified directly by reverse phase liquid chromatography to give 320-8 (413 mg, 0.292 mmol, 42.81%) as a white solid.

### Step 7

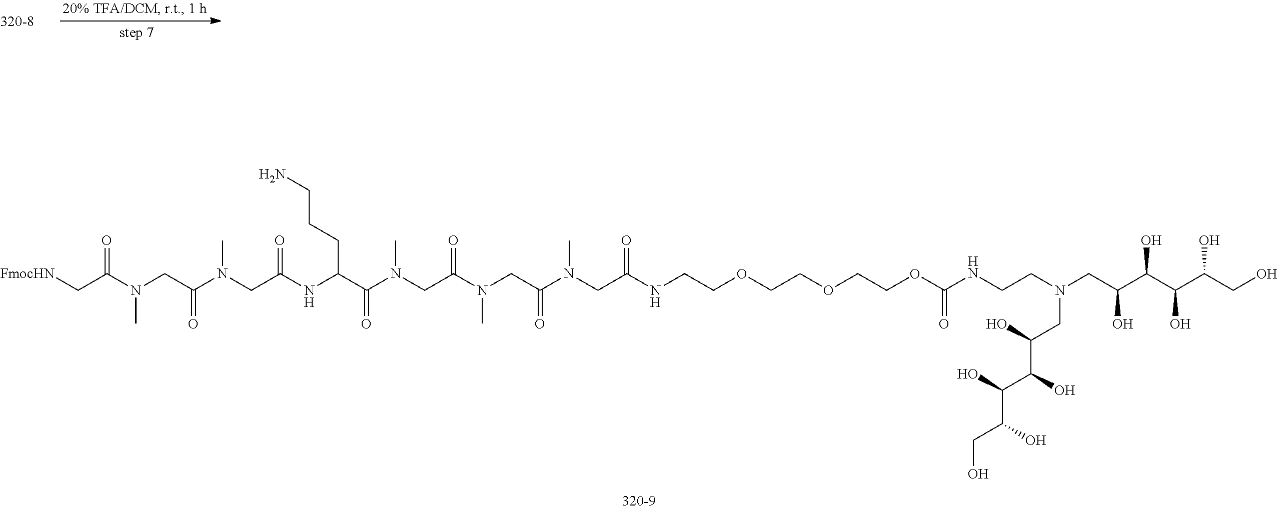

A solution of 320-8 (413 mg, 0.292 mmol) and TFA (2 mL) in anhydrous DCM (8 mL) was stirred at room temperature for 1 hr until LCMS indicated all starting amine was disappeared and desired product was detected. Then the solution was concentrated to dryness to 320-9 (370 mg, 0.262 mmol, 89.70%) as yellow oil, used as such in the next step.

### Step 8

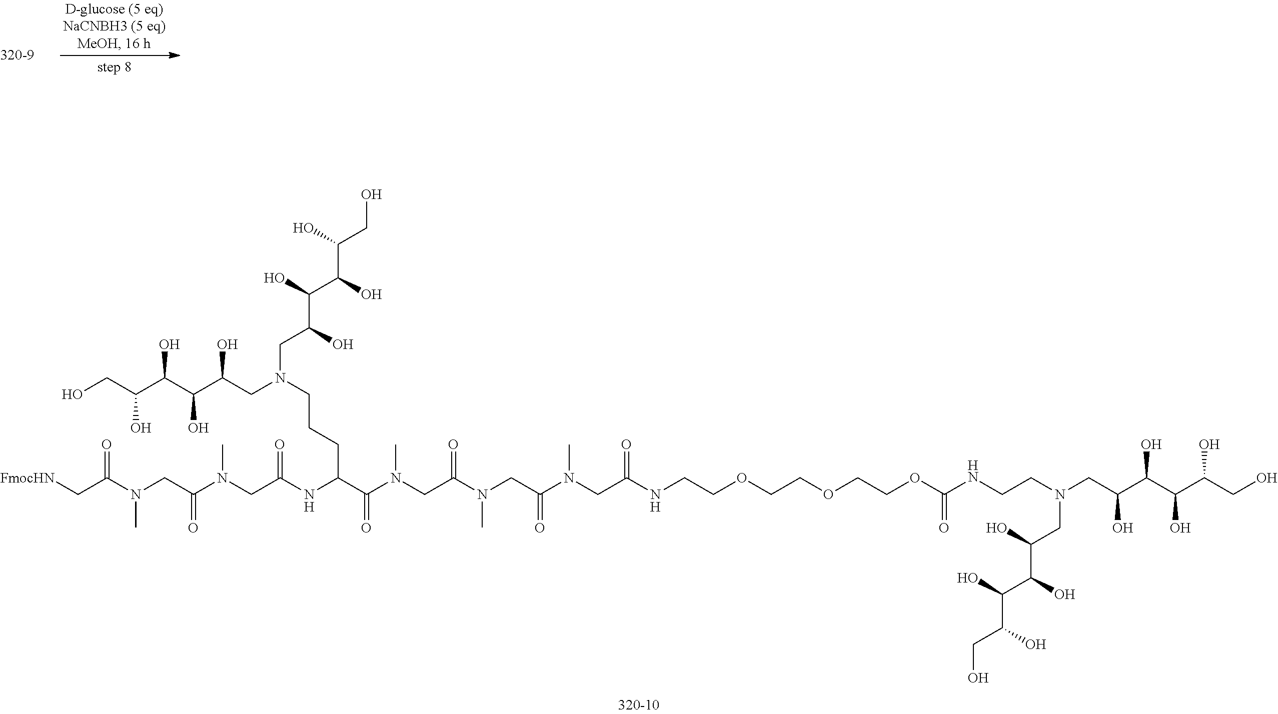

A solution of 320-9 (370 mg, 0.262 mmol) and D-Glucose (203 mg, 1.127 mmol) in anhydrous Methanol (40 mL) was heated at 50° C. for 30 min, then NaCNBH₃ (71 mg, 1.130 mmol) was added. The resulting solution was stirred for another 16 hr at 70° C. until indicated all starting amine was disappeared and the mass of desired product was detected. Then the reaction solution was concentrated and purified by reverse phase liquid chromatography to give 320-10 (210 mg, 0.128 mmol, 48.85%) as a white solid.

### Step 9

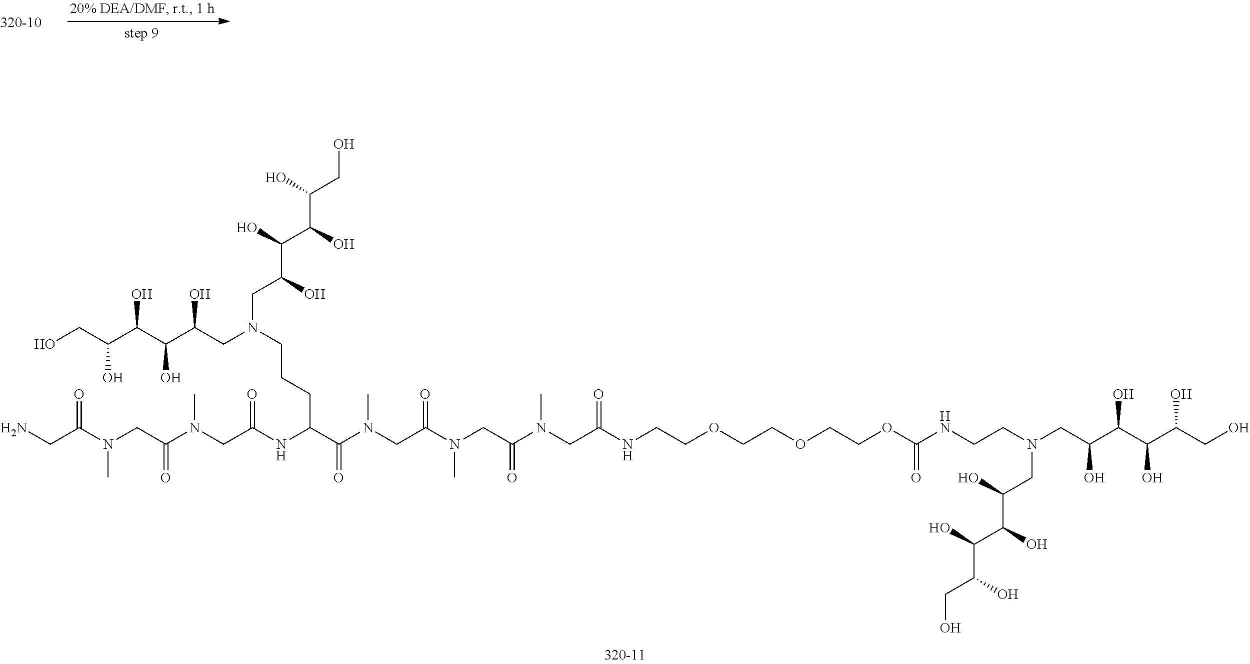 [SMILES: CN(CC(=O)NCCOCCOCCOC(=O)NCCN(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)C(CCCN(C[C@H](O)[C@@H](O)[C@H](O)[C@H](O)CO)C[C@H](O)[C@@H](O)[C@H](O)[C@H](O)CO)NC(=O)CN(C)C(=O)CN(C)C(=O)CN]

A solution of 320-10 (210 mg, 0.128 mmol) and DEA (1 mL) in anhydrous DMF (4 mL) was stirred at room temperature for 1 hr until LCMS indicated all starting amine was disappeared and desired product was detected. Then the solution was concentrated to dryness to 320-11 (175 mg, 0.123 mmol, 96.38%) as colorless oil, used as such in the next step.

### Example 14: Preparation of an Exemplary Polar Group

### Step 1

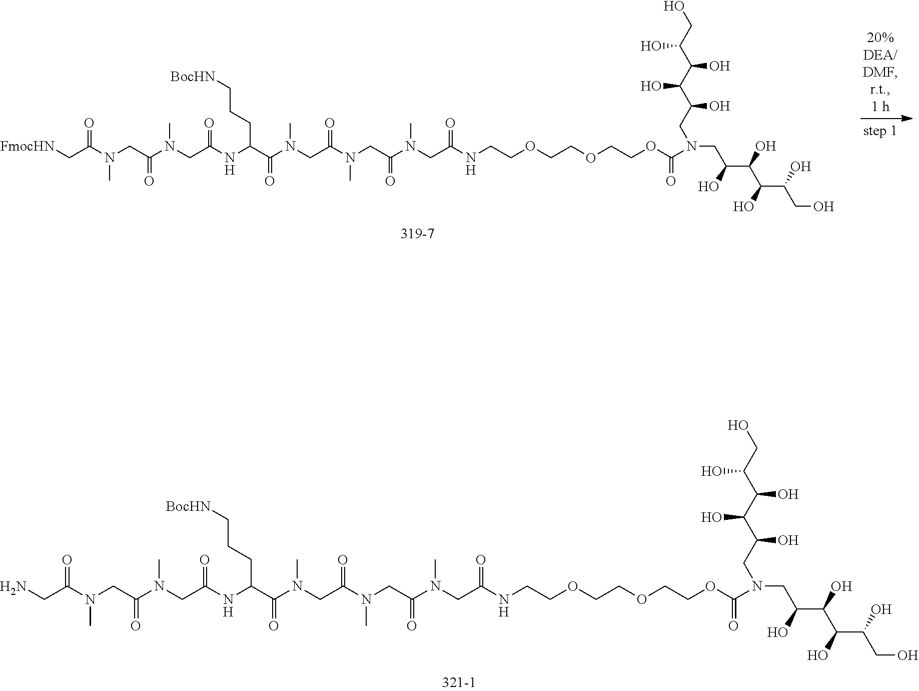

A solution of 319-7 (350 mg, 0.250 mmol) and DEA (1 mL) in anhydrous DMF (4 mL) was stirred at room temperature for 1 hour until LCMS indicated all starting amine was disappeared and desired product was detected. Then the solution was concentrated to dryness to 321-1 (280 mg, 0.238 mmol, 95.29%) as colorless oil, used as such in the next step.

### Step 2

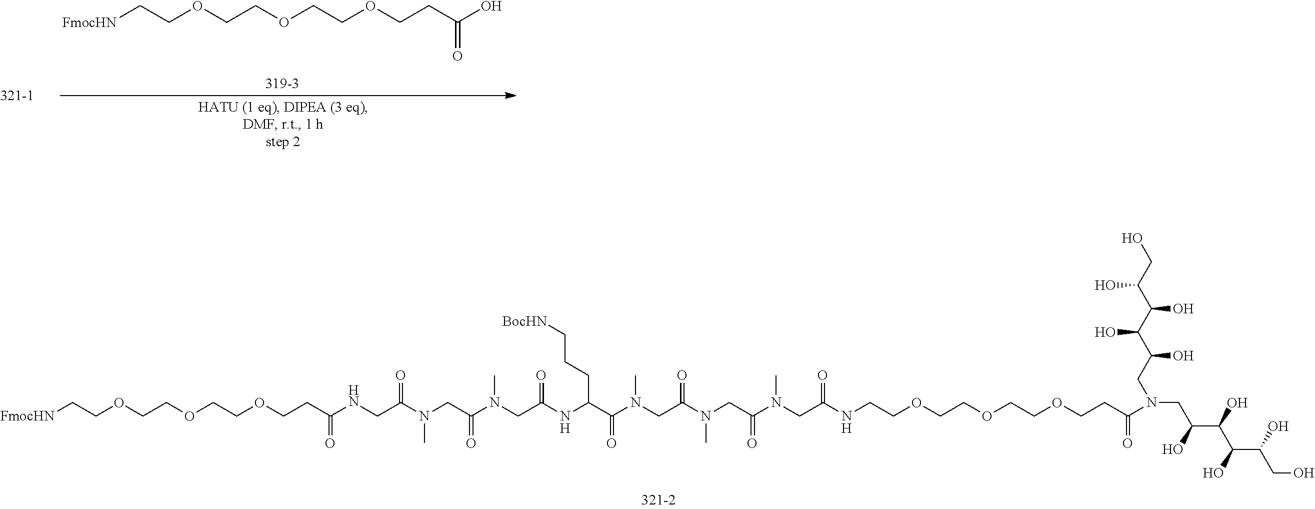

A solution of 321-1 (280 mg, 0.238 mmol), 319-3 (105 mg, 0.237 mmol) and HATU (90 mg, 0.237 mmol) in anhydrous DMF (10 mL) was stirred at room temperature for 5 min, then DIPEA (92 mg, 0.712 mmol) was added. The resulting solution was stirred for another 1 hour at r.t. until LCMS indicated complete reaction. The reaction solution was purified directly by reverse phase liquid chromatography to give 321-2 (265 mg, 0.166 mmol, 69.56%) as a white solid.

### Step 3

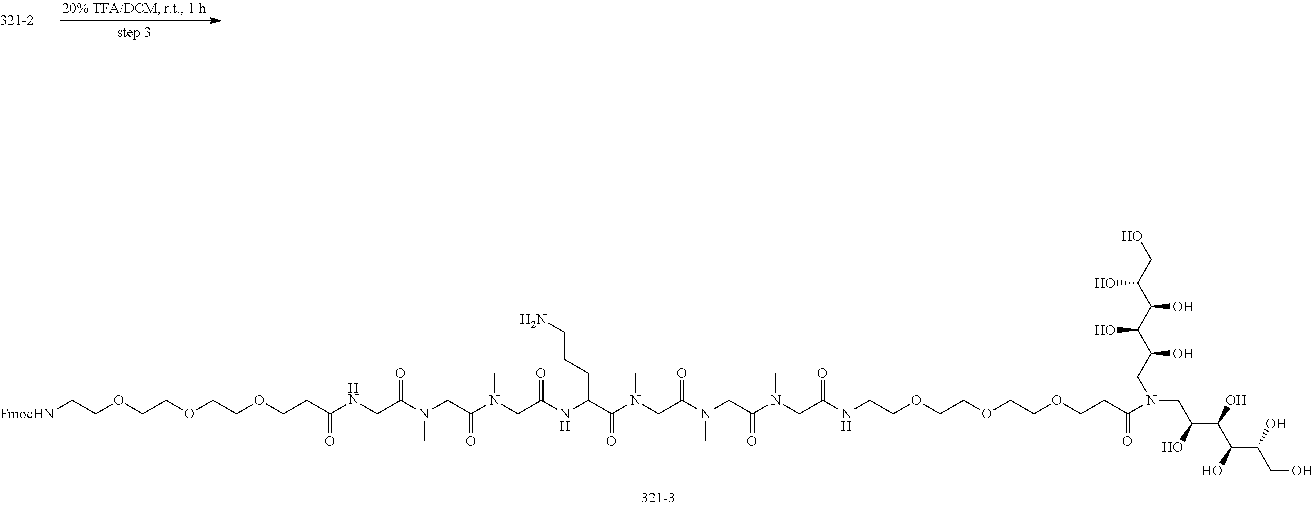

A solution of 321-2 (265 mg, 0.166 mmol) and TFA (1 mL) in anhydrous DCM (4 mL) was stirred at room temperature for 1 hr until LCMS indicated all starting amine was disappeared and desired product was detected. Then the solution was concentrated to dryness to 321-3 (243 mg, 0.162 mmol, 97.55%) as yellow oil, used as such in the next step.

### Step 4

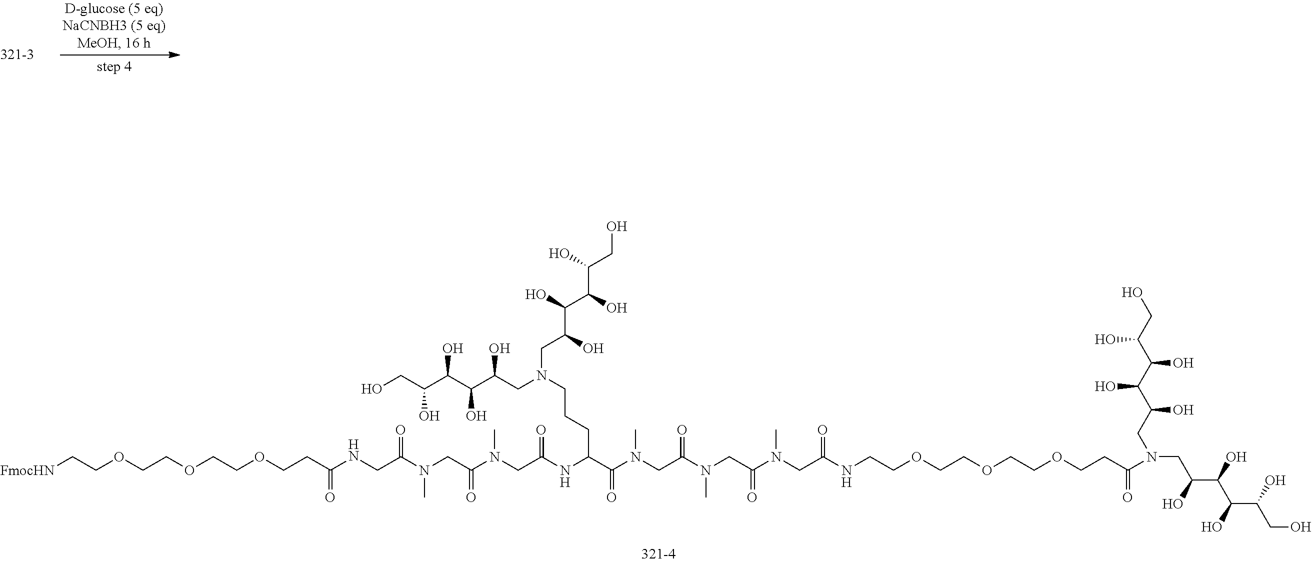

A solution of 321-3 (243 mg, 0.162 mmol) and D-Glucose (146 mg, 0.810 mmol) in anhydrous Methanol (50 mL) was heated at 50° C. for 30 min, then NaCNBH₃ (51 mg, 0.812 mmol) was added. The resulting solution was stirred for another 16 hr at 70° C. until indicated all starting amine was disappeared and the mass of desired product was detected. Then the reaction solution was concentrated and purified by reverse phase liquid chromatography to give 321-4 (155 mg, 0.085 mmol, 52.31%) as a white solid.

### Step 5

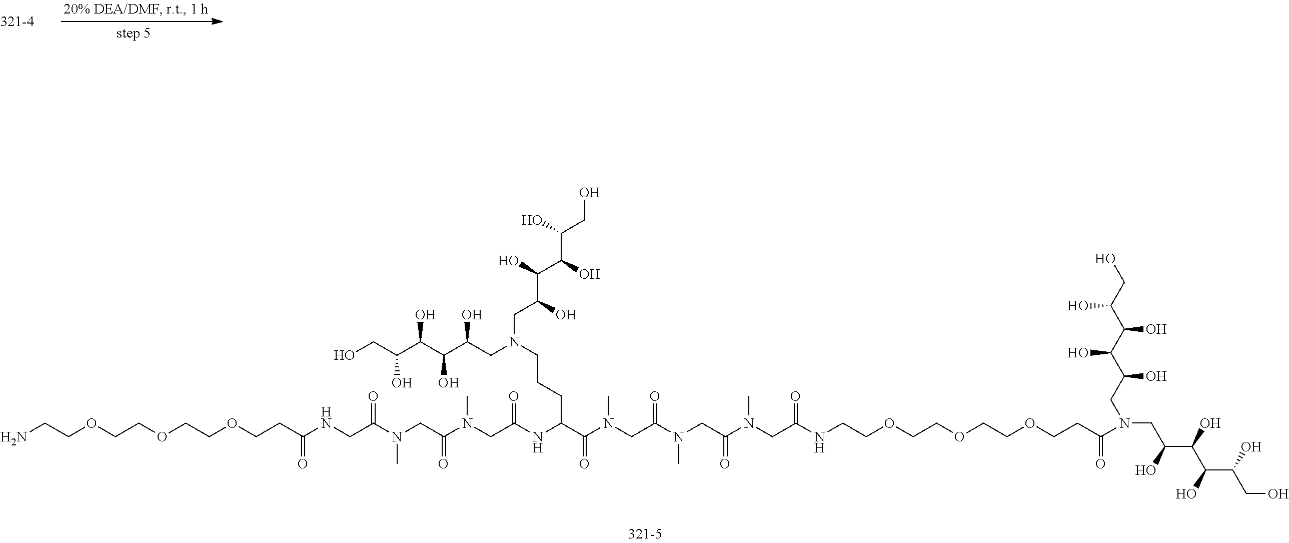 [SMILES: CN(CC(=O)NCCOCCOCCOCCC(=O)N(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)C(CCCN(C[C@H](O)[C@@H](O)[C@H](O)[C@H](O)CO)C[C@H](O)[C@@H](O)[C@H](O)[C@H](O)CO)NC(=O)CN(C)C(=O)CN(C)C(=O)CNC(=O)CCOCCOCCOCCN]

A solution of 321-4 (155 mg, 0.085 mmol) and DEA (1 mL) in anhydrous DMF (4 mL) was stirred at room temperature for 1 hr until LCMS indicated all starting amine was disappeared and desired product was detected. Then the solution was concentrated to dryness to 321-5 (130 mg, 0.081 mmol, 95.19%) as colorless oil, used as such in the next step.

### Example 15: Preparation of an Exemplary Polar Group

### Step 1

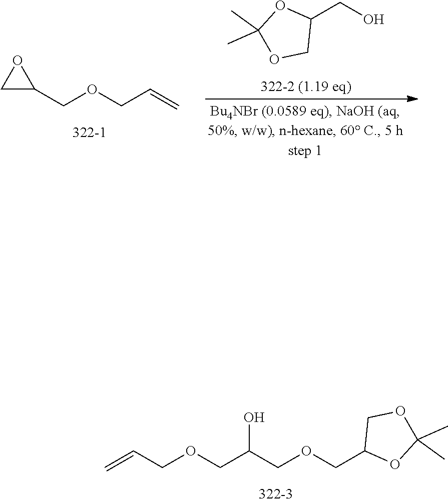

A solution of 322-2 (12.5 g, 94.64 mmol) and Bu₄NBr (1.51 g, 4.684 mmol) in mixture solvent of n-hexane (62.6 mL) and NaOH (aq, 50% w/w, 62.5 mL) was stirred at 60° C. A solution of 322-1 (9.07 g, 79.53 mmol) in n-hexane (12.5 mL) was dropped to the reaction mixture, and the resulting mixture was stirred at 60° C. for 5 hrs until most of 322-1 was consumed by TLC. The mixture was cooled down to r.t. and diluted with water (100 mL), then it was extracted with MTBE (200 mL×2). The organic parts were combined, dried over anhydrous Na₂SO₄, filtered and concentrated to dryness under reduced pressure. The crude product was purified with column chromatography (silica, 0-50% Ethyl acetate in petroleum ether) affording 322-3 as a colorless oil 20.68 g. ¹H NMR (400 MHz, CDCl₃) δ 5.93-5.80 (m, 1H), 5.30-5.20 (m, 1H), 5.19-5.12 (m, 1H), 4.30-4.15 (m, 1H), 4.05-3.91 (m, 4H), 3.73-3.66 (m, 1H), 3.58-3.43 (m, 6H), 2.74 (s, 1H), 1.39 (d, J=3.6 Hz, 3H), 1.33 (d, J=2.8 Hz, 3H).

### Step 2

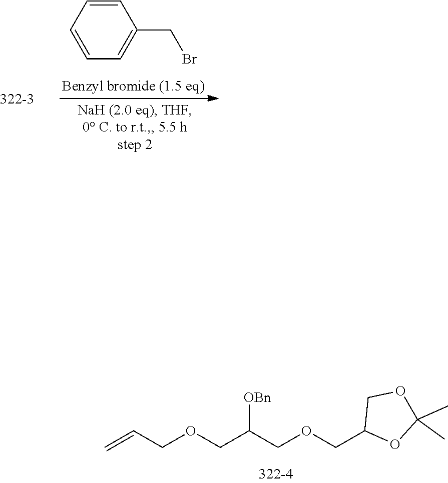 [SMILES: BrCc1ccccc1 ; C=CCOCC(COCC1COC(C)(C)O1)OCc1ccccc1]

To a mixture of NaH (5.58 g, 139.59 mmol) in anhydrous THF (340 mL) cooled in an ice bath was added a solution of 322-3 (17.18 g, 69.79 mmol) in anhydrous THF (85 mL) dropwise, then it was stirred at this temperature for 20 mins. Benzyl bromide (17.79 g, 104.69 mmol) was added dropwise, then the resulting mixture was allowed to warm to r.t. and stirred for 5 hrs until 322-3 was consumed by TLC. The reaction was quenched with saturated ammonium (150 mL) and extracted with ethyl acetate (200 mL×2). The organic phase was combined, dried over anhydrous Na₂SO₄, filtered and concentrated to dryness under reduced pressure to give the crude product, which was purified with column chromatography (silica, 0-40% ethyl acetate in petroleum ether) affording 322-4 (14.7 g, 43.73 mmol, 62.7%) as a colorless oil. Purity=90%-95%. ¹H NMR (400 MHz, CDCl₃) δ 7.41-7.22 (m, 5H), 5.97-5.81 (m, 1H), 5.27 (dt, J=17.2, 1.8 Hz, 1H), 5.17 (dq, J=10.5, 1.5 Hz, 1H), 4.75-4.62 (m, 2H), 4.24 (t, J=6.0 Hz, 1H), 4.07-3.95 (m, 3H), 3.79-3.70 (m, 2H), 3.68-3.59 (m, 2H), 3.58-3.52 (m, 3H), 3.51-3.45 (m, 1H), 1.41 (s, 3H), 1.35 (s, 3H).

### Step 3

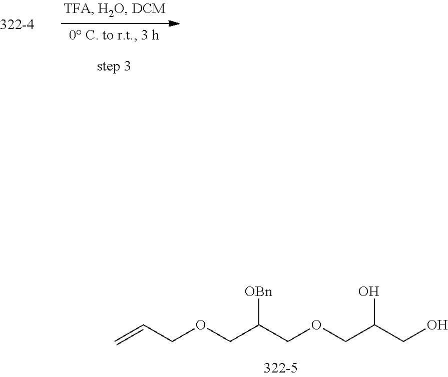 [SMILES: C=CCOCC(COCC(O)CO)OCc1ccccc1]

A solution of 322-4 (3.5 g, 10.411 mmol) in DCM (62.3 mL) and H₂O (0.78 mL) was stirred in an ice bath, then trifluoroacetic acid (1.56 mL) was added. The resulting solution was stirred for 3 hrs at this temperature until 322-4 was consumed by TLC. The reaction was quenched with saturated NaHCO₃ (aq) and then diluted with water (20 mL). The mixture was extracted with DCM (30 mL×2) and the organic phase was combined, dried over anhydrous Na₂SO₄, filtered and concentrated to dryness under reduced pressure to give the crude product, which was purified with column chromatography (silica, 0-60% Ethyl acetate in petroleum ether) affording 322-5 (2.815 g, 9.505 mmol, 91.3%) as a colorless oil. Purity=90%-95%. ¹H NMR (400 MHz, CDCl₃) δ 7.40-7.26 (m, 5H), 5.95-5.81 (m, 1H), 5.31-5.23 (m, 1H), 5.21-5.14 (m, 1H), 4.73-4.59 (m, 2H), 3.99 (dt, J=5.6, 1.5 Hz, 2H), 3.85-3.78 (m, 1H), 3.76-3.71 (m, 1H), 3.66-3.49 (m, 8H), 2.55 (s, 2H).

### Step 4

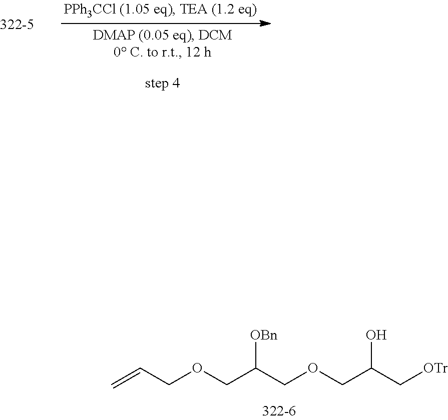

A solution of 322-5 (8.17 g, 27.586 mmol), TEA (3.349 g, 33.103 mmol) and DMAP (168.5 mg, 1.379 mmol) in DCM (40.8 mL) was stirred in an ice bath, then a solution of Ph₃CCl (8.075 g, 28.966 mmol) in DCM (40.8 mL) was added dropwise. The resulting mixture was gradually warmed to room temperature and stirred at ambient temperature for 12 hrs until most of 322-5 was consumed by TLC. The mixture was diluted with DCM (20 mL), washed with water (20 mL×2), dried over anhydrous Na₂SO₄, filtered and concentrated to dryness under reduced pressure to give the crude product, which was purified with column chromatography (silica, 0-40% Ethyl acetate in petroleum ether) affording 322-6 (13.86 g, 25.749 mmol, 93.3%) as a pale yellow oil. Purity=90%-95%. ¹H NMR (400 MHz, CDCl₃) δ 7.47-7.36 (m, 6H), 7.32-7.20 (m, 14H), 5.94-5.90 (m, 1H), 5.29-5.21 (m, 1H), 5.16 (dd, J=10.4, 1.6 Hz, 1H), 4.69-4.57 (m, 2H), 4.0-3.86 (m, 3H), 3.73-3.67 (m, 1H), 3.63-3.55 (m, 3H), 3.55-3.48 (m, 3H), 3.24-3.12 (m, 2H), 2.61 (s, 1H).

### Step 5

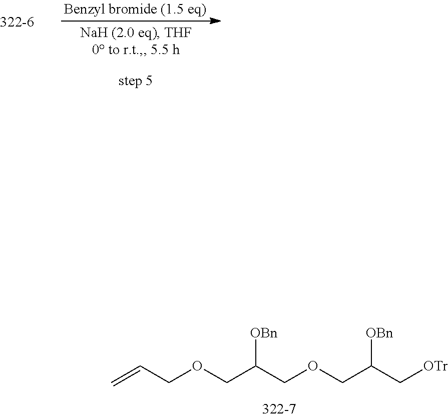

A mixture of NaH (2.06 g, 51.498 mmol) in anhydrous THF (90 mL) was stirred in an ice bath, then a solution of 322-6 (13.86 g, 25.749 mmol) in anhydrous THF (45 mL) was added dropwise. The reaction mixture was stirred at this temperature for 20 mins, then benzyl bromide (6.56 g, 38.62 mmol) was added. The resulting mixture was allowed to warm to r.t. and stirred for 5 hrs until 322-6 was consumed by TLC. The reaction was quenched with saturated ammonium (50 mL) and extracted with (ethyl acetate 60 mL×2). The organic phase was combined, dried over anhydrous Na₂SO₄, filtered and concentrated to dryness under reduced pressure to give the crude product, which was purified with column chromatography (silica, 0-30% ethyl acetate in petroleum ether) affording 322-7 (15.67 g, 24.940 mmol, 96.86%) as a colorless oil. Purity=90%-95%. ¹H NMR (400 MHz, CDCl₃) δ 7.51-7.40 (m, 6H), 7.35-7.20 (m, 19H), 5.94-5.80 (m, 1H), 5.29-5.21 (m, 1H), 5.17-5.10 (m, 1H), 4.65 (d, J=12.1 Hz, 4H), 3.99-3.87 (m, 2H), 3.79-3.65 (m, 2H), 3.65-3.59 (m, 2H), 3.58-3.53 (m, 2H), 3.52-3.46 (m, 2H), 3.23 (d, J=5.0 Hz, 2H).

### Step 6

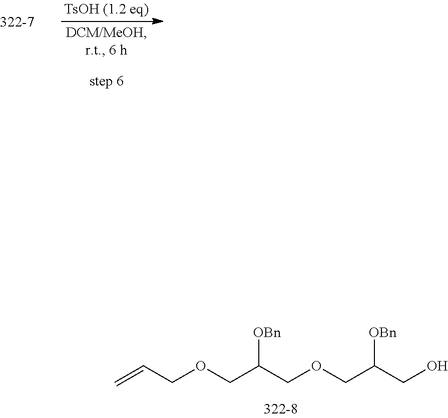 [SMILES: C=CCOCC(COCC(CO)OCc1ccccc1)OCc1ccccc1]

To a solution of 322-7 (15.67 g, 24.939 mmol) in a mixture solvent of DCM (62 mL) and MeOH (31 mL) was added TsOH·H₂O (5.7 g, 29.927 mmol), the resulting mixture was stirred for 6 hrs at room temperature until 322-7 was consumed by TLC. The reaction was quenched with saturated NaHCO₃ (aq.), diluted with water (30 mL), and then extracted with DCM (50 mL×3). The organic phase was combined, dried over anhydrous Na₂SO₄, filtered and concentrated to dryness under reduced pressure to give the crude product, which was purified with column chromatography (silica, 0-50% ethyl acetate in petroleum ether) affording 322-8 (9.09 g, 23.54 mmol, 94.37%) as a pale yellow oil. Purity=90%-95%. ¹H NMR (400 MHz, CDCl₃) δ 7.48-7.15 (m, 10H), 5.98-5.80 (m, 1H), 5.26 (d, J=17.2 Hz, 1H), 5.17 (d, J=10.4 Hz, 1H), 4.75-4.53 (m, 4H), 3.99 (d, J=5.6 Hz, 2H), 3.78-3.70 (m, 2H), 3.68-3.48 (m, 8H).

### Step 7

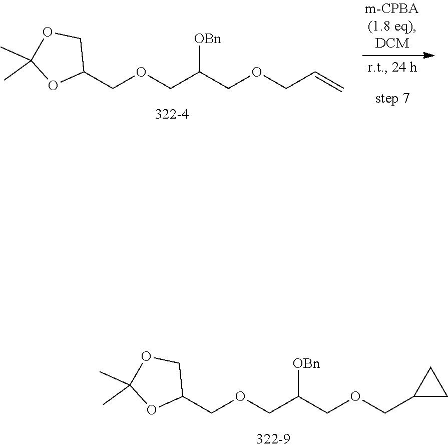

To a solution of 322-4 (14.0 g, 41.555 mmol) in DCM (36 mL) was added a solution of m-CPBA (12.91 g, 74.799 mmol) in DCM (103 mL) dropwise at room temperature. The resulting mixture was stirred at this temperature for 24 hours until 322-4 was consumed, and the reaction was quenched with saturated Na₂S₂O₃ (aq., 50 mL) and NaHCO₃ (aq, 50 mL). The reaction mixture was stirred for 30 mins, and then diluted with DCM (150 mL). The organic phase was washed with a mixture solution (60 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-60% Ethyl acetate in petroleum ether) affording 322-9 (13.79 g, 39.16 mmol, 94.37%) as a pale yellow oil. Purity=90%-95%. ¹H NMR (400 MHz, CDCl₃) δ 7.45-7.16 (m, 5H), 4.69 (s, 2H), 4.25 (t, J=5.6 Hz, 1H), 4.04 (t, J=7.2 Hz, 1H), 3.86-3.69 (m, 3H), 3.68-3.36 (m, 7H), 3.19-3.08 (m, 1H), 2.83-2.76 (m, 1H), 2.63-2.53 (m, 1H), 1.42 (s, 3H), 1.37 (s, 3H).

### Step 8

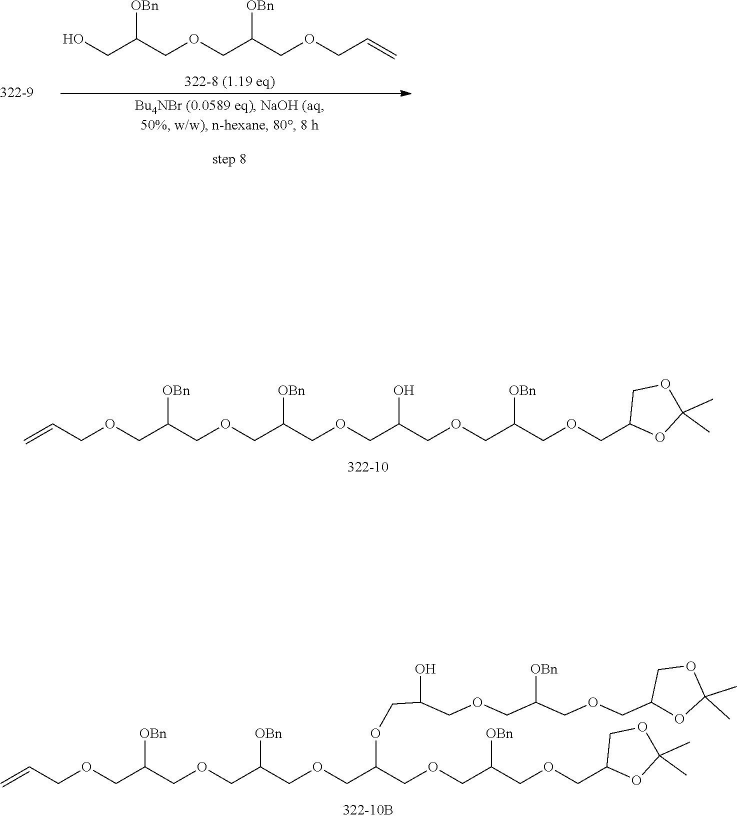

A solution of 322-8 (8.429 g, 23.934 mmol) and Bu₄NBr (454.4 mg, 1.409 mmol) in mixture solvent of n-hexane (30 mL) and NaOH (aq, 50% w/w, 30 mL) was stirred at 80° C. A mixture of 322-9 (11.0 g, 28.482 mmol) in n-hexane (6 mL) was dropped to the reaction mixture, and the resulting mixture was stirred at 80° C. for 8 hrs until most of 322-9 was consumed by TLC. The mixture was cooled down to r.t. and diluted with water (50 mL), then it was extracted with MTBE (100 mL×3). The organic parts were combined, dried over anhydrous Na₂SO₄, filtered and concentrated to dryness under reduced pressure. The crude product was purified with column chromatography (silica, 0-100% ethyl acetate in petroleum ether) affording 322-10 (8.0 g, 10.83 mmol, 45.3%) as a pale yellow oil and 322-10B (2.1 g, 1.925 mmol, 8.0%) as a pale yellow oil. ¹H NMR (400 MHz, CDCl₃) δ 7.38-7.15 (m, 15H), 5.96-5.73 (m, 1H), 5.31-5.06 (m, 2H), 4.76-4.51 (m, 6H), 4.29-4.13 (m, 1H), 4.07-3.86 (m, 4H), 3.76-3.37 (m, 22H), 2.52 (s, 1H), 1.56-1.05 (m, 6H). ¹H NMR (400 MHz, CDCl₃) δ 7.42-7.27 (m, 20H), 5.96-5.82 (m, 1H), 5.26 (dd, J=17.2, 1.8 Hz, 1H), 5.16 (dd, J=10.5, 1.9 Hz, 1H), 4.69-4.61 (m, 8H), 4.27-4.19 (m, 2H), 4.05-3.97 (m, 4H), 3.90-3.80 (m, 2H), 3.76-3.66 (m, 8H), 3.61-3.48 (m, 26H), 3.09 (s, 1H), 1.58-1.16 (m, 12H).

### Step 9

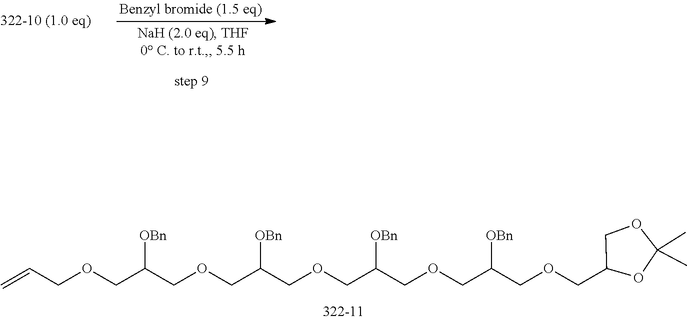 [SMILES: C=CCOCC(COCC(COCC(COCC(COCC1COC(C)(C)O1)OCc1ccccc1)OCc1ccccc1)OCc1ccccc1)OCc1ccccc1]

A mixture of NaH (867 mg, 21.668 mmol) in anhydrous THF (36 mL) was stirred in an ice bath, then a solution of 322-10 (8.0 g, 10.834 mmol) in anhydrous THF (20 mL) was added dropwise. The reaction mixture was stirred at this temperature for 20 mins, then benzyl bromide (2.76 g, 16.251 mmol) was added. The resulting mixture was allowed to warm to r.t. and stirred for 5 hrs until 322-10 was consumed by TLC. The reaction was quenched with saturated ammonium (25 mL) and extracted with ethyl acetate (40 mL×2). The organic phase was combined, dried over anhydrous Na₂SO₄, filtered and concentrated to dryness under reduced pressure to give the crude product, which was purified with column chromatography (silica, 0-60% Ethyl acetate in petroleum ether) affording 322-11 (8.45 g, 10.20 mmol, 94.1%) as a pale yellow oil. Purity=90%-95%. ¹H NMR (400 MHz, CDCl₃) δ 7.43-7.25 (m, 20H), 5.99-5.82 (m, 1H), 5.27 (dd, J=17.2, 1.8 Hz, 1H), 5.17 (dd, J=10.4, 1.7 Hz, 1H), 4.77-4.60 (m, 8H), 4.30-4.18 (m, 1H), 4.06-3.96 (m, 3H), 3.81-3.70 (m, 5H), 3.63-3.47 (m, 18H), 1.47-1.21 (m, 6H).

### Step 10

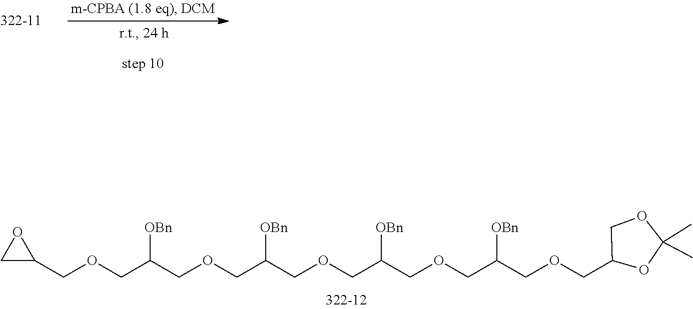 [SMILES: CC1(C)OCC(COCC(COCC(COCC(COCC(COCC2CO2)OCc2ccccc2)OCc2ccccc2)OCc2ccccc2)OCc2ccccc2)O1]

To a solution of 322-11 (8.45 g, 10.20 mmol) in DCM (12.8 mL) was added a solution of m-CPBA (3.16 g, 18.36 mmol) in DCM (25.6 mL) dropwise at room temperature. The resulting mixture was stirred at this temperature for 24 hours until 322-11 was consumed, and the reaction was quenched with saturated Na₂S2O₃ (aq, 15 mL) and NaHCO₃ (aq, 15 mL). The reaction mixture was stirred for 30 mins, and then diluted with DCM (120 mL). The organic phase was washed with a mixture solution (40 mL×3) of saturated Na₂S2O₃ and NaHCO₃ (aq, 1:1, V/V), dried over anhydrous Na₂SO₄, filtered and concentrated to dryness under reduced pressure to give the crude product, which was purified with column chromatography (silica, 0-60% ethyl acetate in petroleum ether) affording 322-12 (6.858 g, 8.12 mmol, 79.6%) as a pale yellow oil. Purity=90%-95%. ¹H NMR (400 MHz, CDCl₃) δ 7.53-7.25 (m, 20H), 4.68 (s, 8H), 4.28-4.20 (m, 1H), 4.06-4.00 (m, 1H), 3.80-3.69 (m, 6H), 3.66-3.50 (m, 16H), 3.49-3.44 (m, 1H), 3.41-3.35 (m, 1H), 3.16-3.08 (m, 1H), 2.76 (t, J=4.6 Hz, 1H), 2.63-2.52 (m, 1H), 1.54-1.24 (m, 6H).

### Step 11

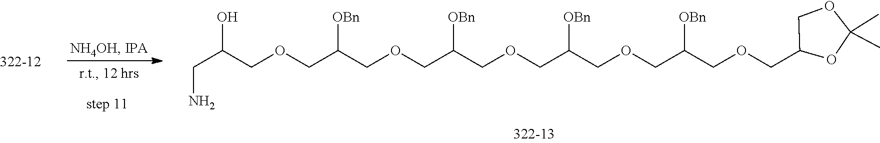 [SMILES: CC1(C)OCC(COCC(COCC(COCC(COCC(COCC(O)CN)OCc2ccccc2)OCc2ccccc2)OCc2ccccc2)OCc2ccccc2)O1]

To a solution of 322-12 (1.22 g, 1.445 mmol) in isopropyl alcohol (120 mL) was added ammonia (120 mL) dropwise at room temperature, and the resulting mixture was stirred at this temperature for 12 hours until 322-12 was consumed. The reaction mixture was concentrated to dryness under reduced pressure to afford 322-13 (1.28 g, 1.48 mmol, 100%) as a colorless oil, which was used in the next step without further purification. Purity=90%-95%.

### Step 12

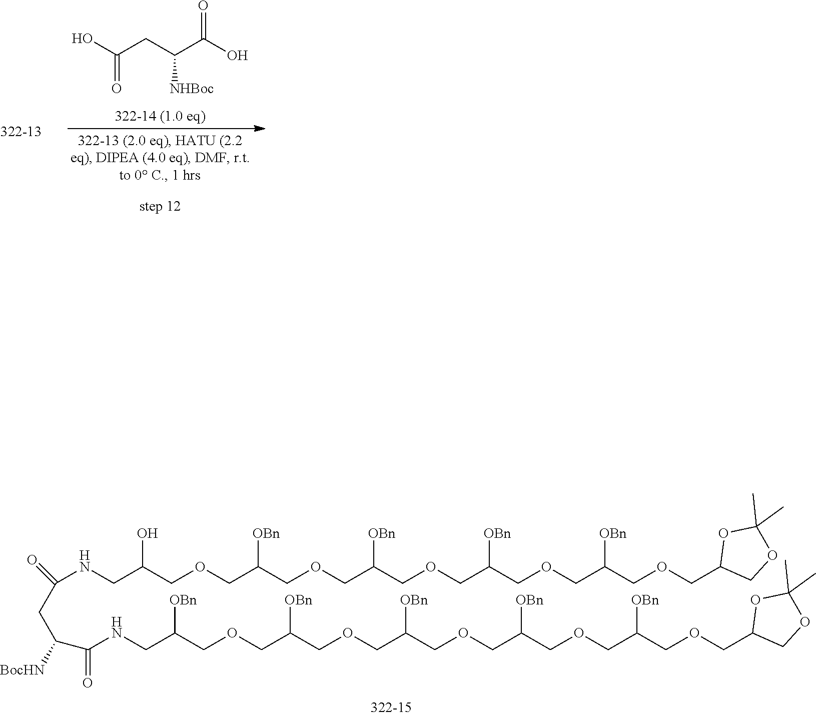 [SMILES: CC(C)(C)OC(=O)N[C@H](CC(=O)O)C(=O)O ; CC(C)(C)OC(=O)N[C@H](CC(=O)NCC(O)COCC(COCC(COCC(COCC(COCC1COC(C)(C)O1)OCc1ccccc1)OCc1ccccc1)OCc1ccccc1)OCc1ccccc1)C(=O)NCC(COCC(COCC(COCC(COCC(COCC1COC(C)(C)O1)OCc1ccccc1)OCc1ccccc1)OCc1ccccc1)OCc1ccccc1)OCc1ccccc1]

A solution of 322-14 (81.2 mg, 0.348 mmol) and HATU (291 mg, 0.766 mmol) in anhydrous DMF (5 mL) was stirred at room temperature for 15 mins, then it was stirred in an ice bath. A solution of 322-13 (600 mg, 0.696 mmol) in anhydrous DMF (3 mL) was added dropwise, followed by DIPEA (180 mg, 1.392 mmol). The resulting mixture was stirred in the ice bath for 1 h until most of 322-14 was consumed, and then diluted with ethyl acetate (60 mL). The organic phase was washed with H₂O (20 mL×3), dried over anhydrous Na₂SO₄, filtered and concentrated to dryness under reduced pressure to give the crude product, which was purified with column chromatography (silica, 0-100% ethyl acetate in petroleum ether) affording 322-15 (660 mg, 0.343 mmol, 98.7%) as a pale yellow oil. Purity=90%-95%. ¹H NMR (400 MHz, CDCl₃) δ 7.74-7.24 (m, 40H), 6.68-6.40 (m, 1H), 6.25-6.00 (m, 1H), 4.84-4.50 (m, 16H), 4.40 (s, 1H), 4.23 (t, J=6.0 Hz, 2H), 4.05-3.97 (m, 2H), 3.82-3.36 (m, 53H), 3.22-2.96 (m, 3H), 2.93-2.61 (m, 5H), 2.52-2.33 (m, 1H), 1.44-1.26 (m, 21H).

### Step 13

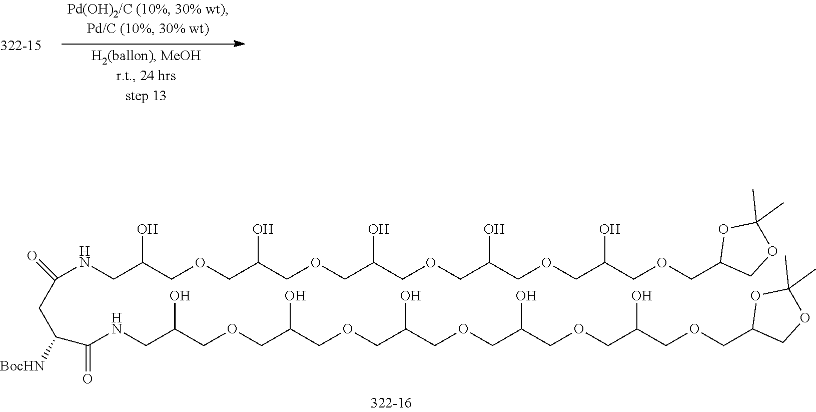 [SMILES: CC(C)(C)OC(=O)N[C@H](CC(=O)NCC(O)COCC(O)COCC(O)COCC(O)COCC(O)COCC1COC(C)(C)O1)C(=O)NCC(O)COCC(O)COCC(O)COCC(O)COCC(O)COCC1COC(C)(C)O1]

A mixture of 322-15 (160 mg, 0.0833 mmol), Pd(OH)₂/C (10%, 50 mg) and Pd/C (10%, 50 mg) in MeOH (10 mL) was stirred under hydrogen atmosphere (ballon) for 24 h at room temperature until 322-15 was completely converted into 322-16. The reaction mixture was filtered through a celite pad, and the filtrate was concentrated to dryness under reduced pressure to afford 322-16 (78.3 mg, 0.0652 mmol, 78.3%) as a colorless oil, which was used in the next step without further purification.

### Step 14

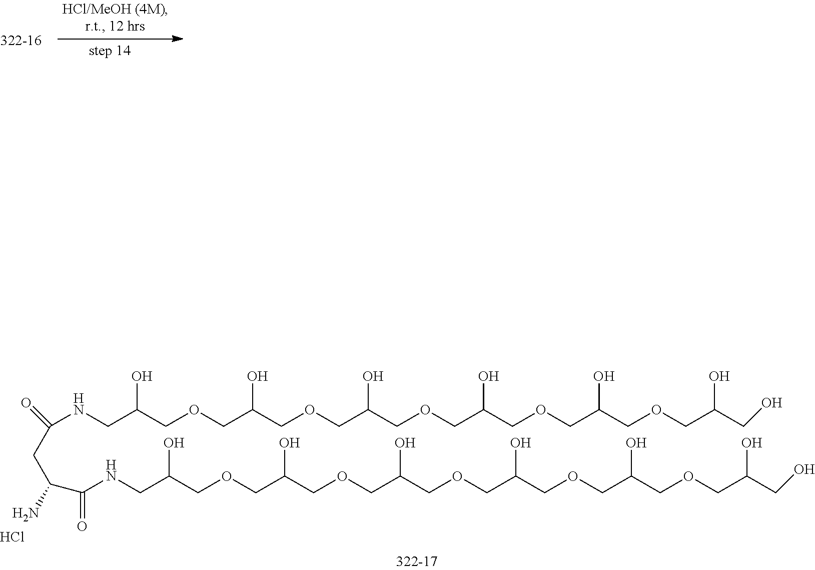 [SMILES: N[C@H](CC(=O)NCC(O)COCC(O)COCC(O)COCC(O)COCC(O)COCC(O)CO)C(=O)NCC(O)COCC(O)COCC(O)COCC(O)COCC(O)COCC(O)CO ; Cl]

A mixture of 322-16 (78.3 mg, 0.0652 mmol) and HCl/MeOH (4M, 4 mL) was stirred at room temperature for 12 hours until 322-16 was completely converted into 322-17. The reaction mixture was concentrated to dryness under reduced pressure to afford 322-17 (65.0 mg, 0.0616 mmol, 94.4%) as an off-white solid, which was used in the next step without further purification.

### Example 16: Preparation of an Exemplary Polar Group

### Step 1

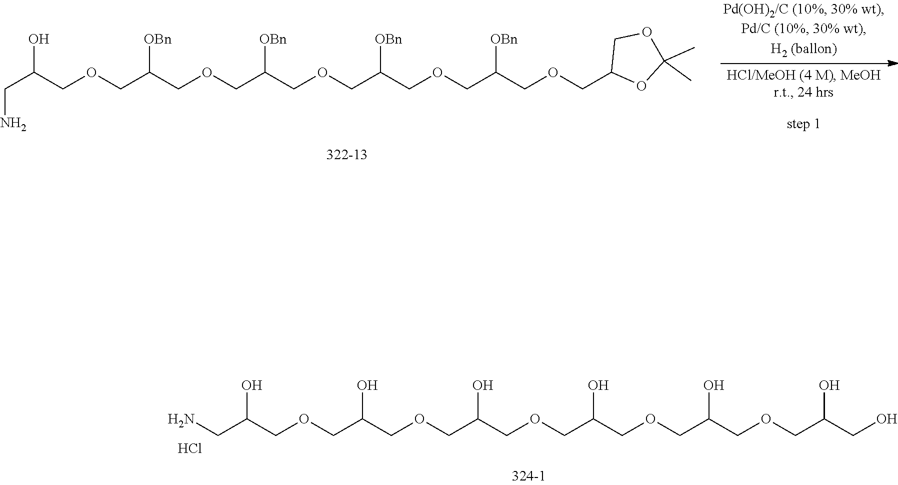 [SMILES: CC1(C)OCC(COCC(COCC(COCC(COCC(COCC(O)CN)OCc2ccccc2)OCc2ccccc2)OCc2ccccc2)OCc2ccccc2)O1 ; NCC(O)COCC(O)COCC(O)COCC(O)COCC(O)COCC(O)CO ; Cl]

A mixture of 322-13 (160 mg, 0.186 mmol), Pd(OH)₂/C (10%, 50 mg) and Pd/C (10%, 50 mg) in a mixed solvent of MeOH (10 mL) and HCl/MeOH (4M, 3 mL) was stirred under hydrogen atmosphere (ballon) for 24 h at room temperature until 322-13 was completely converted into 324-1. The reaction mixture was filtered through a celite pad, and the filtrate was concentrated to dryness under reduced pressure to afford 324-1 (92.3 mg, 0.186 mmol, 100%) as an off-white solid, which was used in the next step without further purification.

### Example 17: Preparation of an Exemplary Polar Group

### Step 1

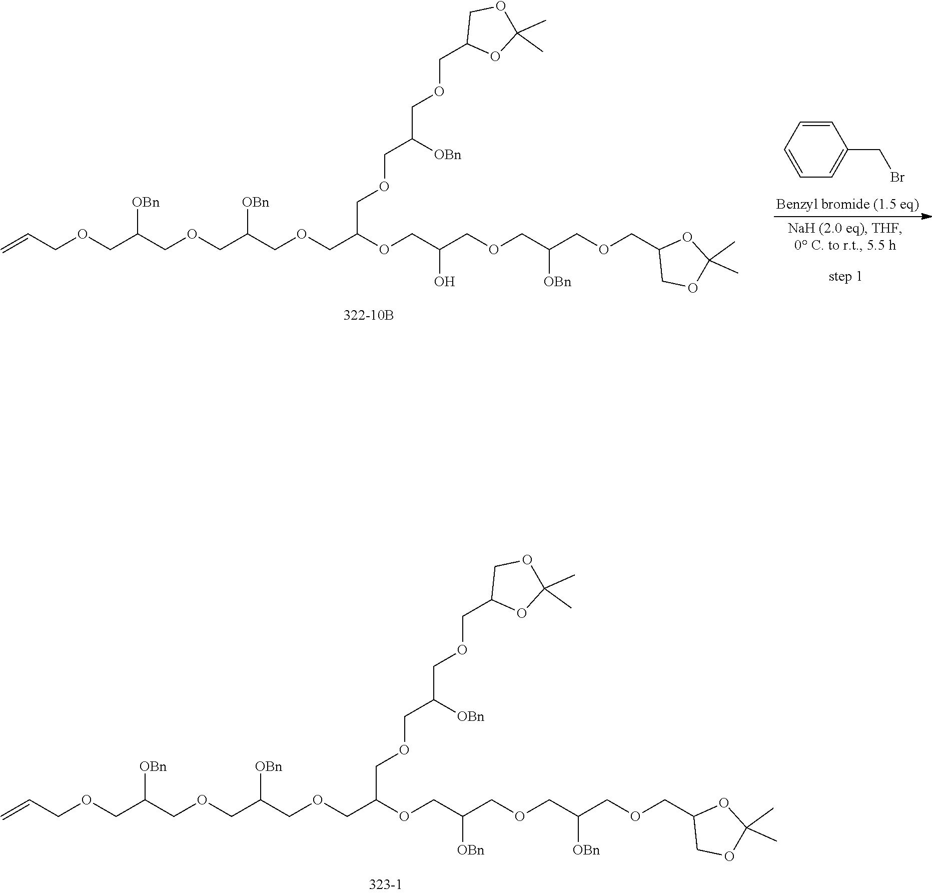

A mixture of NaH (205 mg, 5.13 mmol) in anhydrous THF (12.6 mL) was stirred in an ice bath, then a solution of 322-10B (2.79 g, 2.565 mmol) in anhydrous THF (7 mL) was added dropwise. The reaction mixture was stirred at this temperature for 20 mins, then benzyl bromide (654 g, 3.847 mmol) was added. The resulting mixture was allowed to warm to r.t. and stirred for 5 hrs until 322-10B was consumed by TLC. The reaction was quenched with saturated ammonium (15 mL) and extracted with ethyl acetate (30 mL×2). The organic phase was combined, dried over anhydrous Na₂SO₄, and concentrated to dryness under reduced pressure to give the crude product, which was purified with column chromatography (silica, 0-80% ethyl acetate in petroleum ether) affording 323-1 (2.772 g, 2.348 mmol, 91.8%) as a pale yellow oil. Purity=90%-95%.

### Step 2

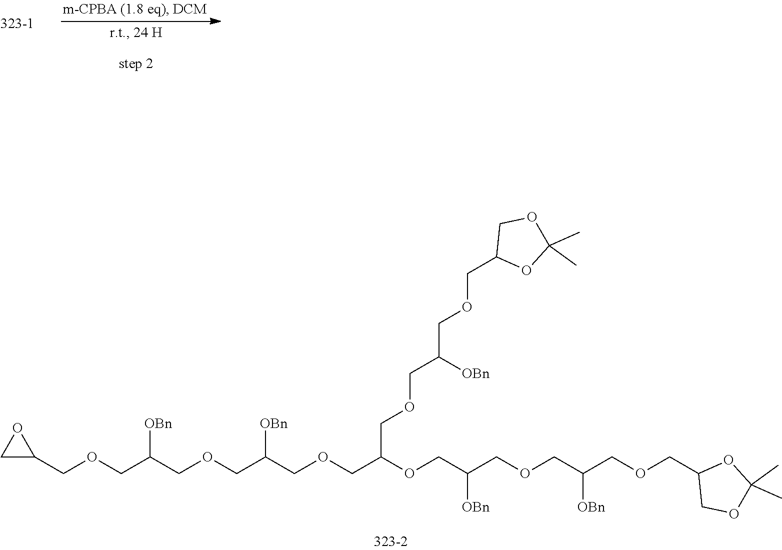 [SMILES: CC1(C)OCC(COCC(COCC(COCC(COCC(COCC2CO2)OCc2ccccc2)OCc2ccccc2)OCC(COCC(COCC2COC(C)(C)O2)OCc2ccccc2)OCc2ccccc2)OCc2ccccc2)O1]

To a solution of 323-1 (2.77 g, 2.346 mmol) in DCM (4.2 mL) was added a solution of m-CPBA (729 mg, 4.223 mmol) in DCM (8.4 mL) dropwise at room temperature. The resulting mixture was stirred at this temperature for 24 hours until 323-1 was consumed, and the reaction was quenched with saturated Na₂S₂O₃ (aq., 10 mL) and NaHCO₃ (aq, 10 mL). The reaction mixture was stirred for 30 mins, and then diluted with DCM (100 mL). The organic phase was washed with a mixture solution (30 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-100% ethyl acetate in petroleum ether) affording 323-2 (2.147 g, 1.79 mmol, 76.5%) as a pale yellow oil. Purity=90%-95%. ¹H NMR (400 MHz, CDCl₃) δ 7.50-7.20 (m, 25H), 4.65 (s, 10H), 4.22 (t, J=6.0 Hz, 2H), 4.06-3.98 (m, 2H), 3.82-3.30 (m, 38H), 3.15-3.03 (m, 1H), 2.82-2.70 (m, 1H), 2.62-2.51 (m, 1H), 1.46-1.28 (m, 12H).

### Step 3

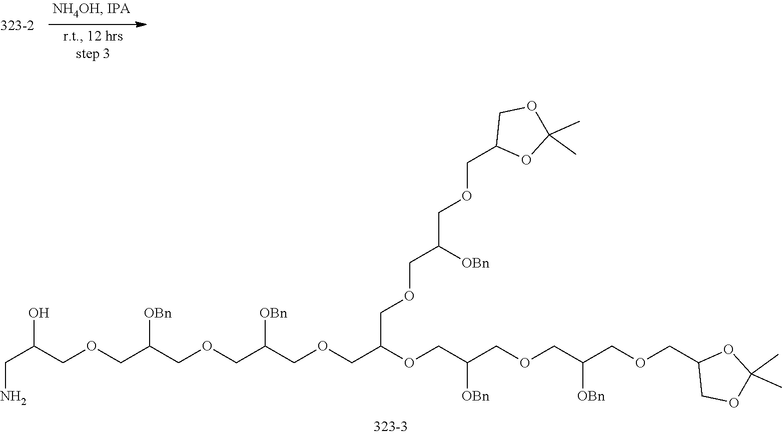 [SMILES: CC1(C)OCC(COCC(COCC(COCC(COCC(COCC(O)CN)OCc2ccccc2)OCc2ccccc2)OCC(COCC(COCC2COC(C)(C)O2)OCc2ccccc2)OCc2ccccc2)OCc2ccccc2)O1]

To a solution of 323-2 (450 mg, 0.376 mmol) in isopropyl alcohol (32 mL) was added ammonia (32 mL) dropwise at room temperature, and the resulting mixture was stirred at this temperature for 12 hours until 323-2 was consumed. The reaction mixture was concentrated to dryness under reduced pressure to afford 323-3 (456 mg, 1.48 mmol, 100%) as a colorless oil, which was used in the next step without further purification. Purity=90%-95%.

### Step 4

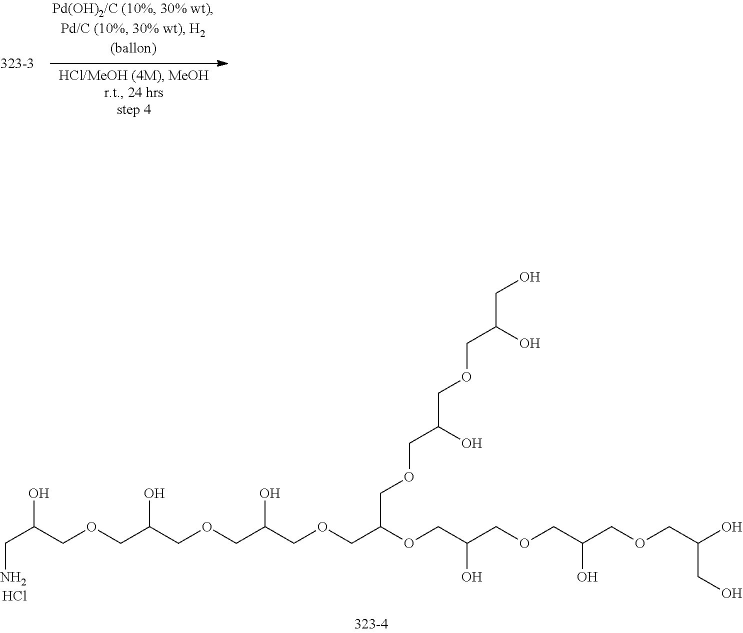 [SMILES: NCC(O)COCC(O)COCC(O)COCC(COCC(O)COCC(O)CO)OCC(O)COCC(O)COCC(O)CO ; Cl]

A mixture of 323-3 (250 mg, 0.206 mmol), Pd(OH)₂/C (10%, 75 mg) and Pd/C (10%, 75 mg) in a mixed solvent of MeOH (16 mL) and HCl/MeOH (4M, 4 mL) was stirred under hydrogen atmosphere (ballon) for 24 h at room temperature until 323-3 was completely converted into 323-4. The reaction mixture was filtered through a celite pad, and the filtered was concentrated to dryness under reduced pressure to afford 323-4 (149.7 mg, 0.208 mmol, 100%) as an off-white solid, which was used in the next step without further purification.

### Example 18: Preparation of an Exemplary Polar Group

### Step 1

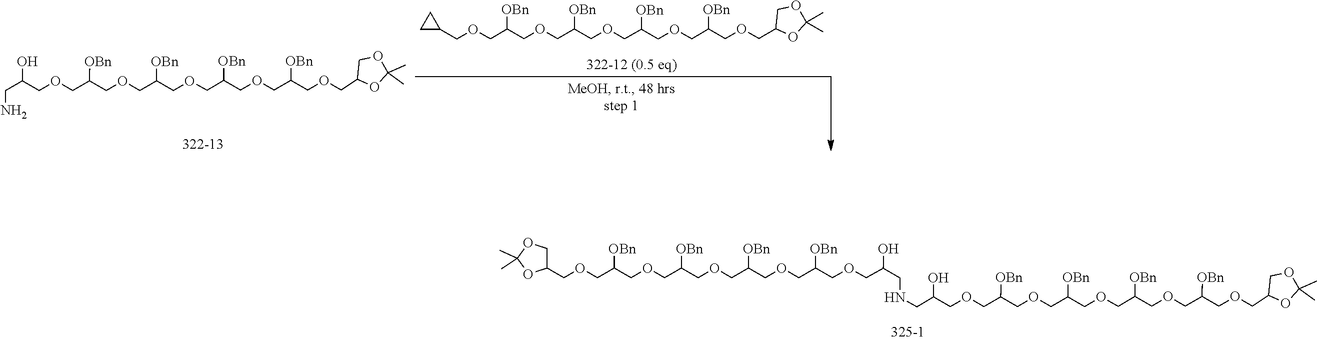

A solution of 322-13 (408 mg, 0.474 mmol), 322-12 (200 mg, 0.237 mmol) and MeOH (20 mL) was stirred at room temperature for 48 hrs. Then the reaction solution was purified by prep-HPLC to afford 325-1 (121.9 mg, 0.0715 mmol, 30.2%) as a colorless oil.

### Step 2

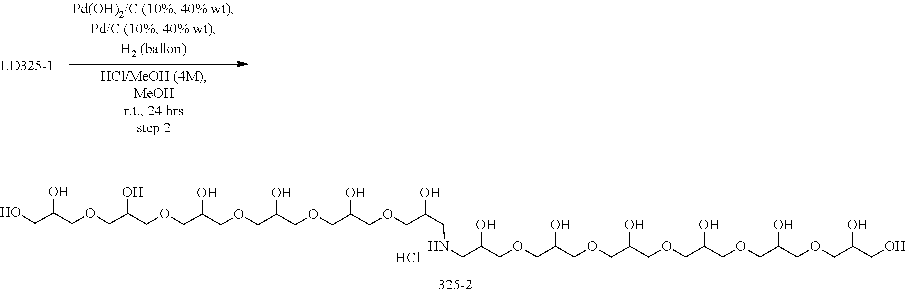 [SMILES: OCC(O)COCC(O)COCC(O)COCC(O)COCC(O)COCC(O)CNCC(O)COCC(O)COCC(O)COCC(O)COCC(O)COCC(O)CO ; Cl]

A mixture of 325-1 (121.9 mg, 0.0715 mmol), Pd(OH)₂/C (10%, 50 mg) and Pd/C (10%, 50 mg) in a mixed solvent of MeOH (10 mL) and HCl/MeOH (4M, 3 mL) was stirred under hydrogen atmosphere (ballon) for 24 h at room temperature until 325-1 was completely converted into 325-2. The reaction mixture was filtered through a celite pad, and the filtrate was concentrated to dryness under reduced pressure to afford 323-4 (67.2 mg, 0.0714 mmol, 100%) as an off-white solid, which was used in the next step without further purification.

### Example 19: Preparation of an Exemplary Polar Group

### Step 1

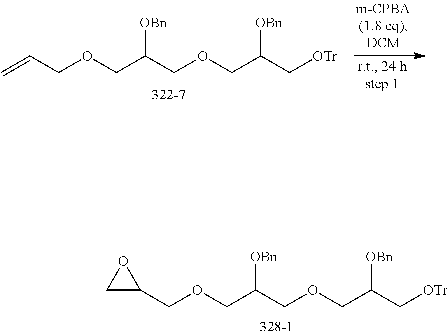

To a solution of 322-7 (10.0 g, 15.915 mmol) in DCM (20 mL) was added a solution of m-CPBA (4.94 g, 28.648 mmol) in DCM (40 mL) dropwise at room temperature. The resulting mixture was stirred at this temperature for 24 hours until 322-7 was consumed, and the reaction was quenched with saturated Na₂S₂O₃ (aq., 20 mL) and NaHCO₃ (aq, 20 mL). The reaction mixture was stirred for 30 mins, and then diluted with DCM (100 mL). The organic phase was washed with a mixture solution (20 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-90% ethyl acetate in petroleum ether) affording 328-1 (9.1 g, 14.12 mmol, 88.7%) as a pale yellow oil. Purity=90%-95%. ¹H NMR (400 MHz, CDCl₃) δ 7.47-7.38 (m, 5H), 7.36-7.03 (m, 20H), 4.70-4.39 (m, 4H), 3.79-3.41 (m, 9H), 3.40-3.32 (m, 1H), 3.28-3.14 (m, 2H), 3.12-3.01 (m, 1H), 2.79-2.66 (m, 1H), 2.60-2.47 (m, 1H).

### Step 2

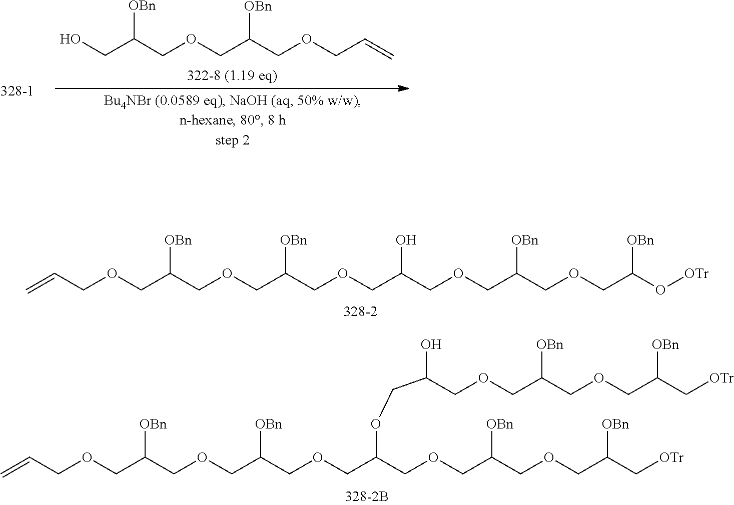

A solution of 322-8 (4.279 g, 11.08 mmol) and Bu₄NBr (177 mg, 0.548 mmol) in mixture solvent of n-hexane (20 mL) and NaOH (aq, 50% w/w, 20 mL) was stirred at 80° C. A mixture of 328-1 (6.0 g, 9.312 mmol) in n-hexane (8 mL) was dropped to the reaction mixture, and the resulting mixture was stirred at 80° C. for 8 hrs until most of 328-1 was consumed by TLC. The mixture was cooled down to r.t. and diluted with water (30 mL), then it was extracted with MTBE (70 mL×3). The organic parts were combined, dried over anhydrous Na₂SO₄, filtered and concentrated to dryness under reduced pressure. The crude product was purified with column chromatography (silica, 0-100% Ethyl acetate in petroleum ether) affording 328-2 (7.22 g, contains 328-2B) as a pale yellow oil. ¹H NMR (400 MHz, CDCl₃) δ 7.48-7.41 (m, 6H), 7.37-7.24 (m, 29H), 5.95-5.81 (m, 1H), 5.29-5.22 (m, 1H), 5.16 (dd, J=10.4, 1.7 Hz, 1H), 4.69-4.63 (m, 6H), 4.62-4.58 (m, 2H), 4.00-3.95 (m, 2H), 3.92-3.85 (m, 1H), 3.76-3.70 (m, 3H), 3.69-3.64 (m, 1H), 3.62-3.50 (m, 15H), 3.48-3.39 (m, 4H), 3.23 (d, J=5.2 Hz, 2H).

### Step 3

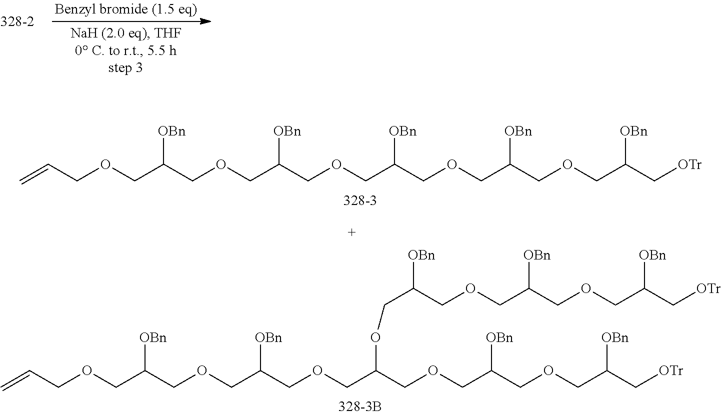

A mixture of NaH (560.5 mg, 14.012 mmol) in anhydrous THF (23.5 mL) was stirred in an ice bath, then a solution of 328-2 (7.22 g, 7.006 mmol, contains 328-2B) in anhydrous THF (13.2 mL) was added dropwise. The reaction mixture was stirred at this temperature for 20 mins, then benzyl bromide (1.786 g, 10.509 mmol) was added. The resulting mixture was allowed to warm to r.t. and stirred for 5 hrs until 328-2 was consumed by TLC. The reaction was quenched with saturated ammonium (20 mL) and extracted with ethyl acetate (40 mL×2). The organic phase was combined, dried over anhydrous Na₂SO₄, filtered and concentrated to dryness under reduced pressure to give the crude product, which was purified with column chromatography (silica, 0-80% Ethyl acetate in petroleum ether) affording 328-3 (5.13 g, 4.578 mmol, 65.3%) as a pale yellow oil and 328-3B (2.0 g, 1.133 mmol) a pale yellow oil. Purity=90%-95%. ¹H NMR (400 MHz, CDCl₃) δ 7.46-7.42 (m, 6H), 7.33-7.20 (m, 34H), 5.94-5.81 (m, 1H), 5.27-5.22 (m, 1H), 5.17-5.12 (m, 1H), 4.67-4.63 (m, 8H), 4.61 (s, 2H), 3.99-3.95 (m, 2H), 3.75-3.66 (m, 5H), 3.60-3.50 (m, 18H), 3.23 (d, J=5.2 Hz, 2H). ¹H NMR (400 MHz, CDCl₃) δ 7.46-7.40 (m, 13H), 7.34-7.25 (m, 27H), 7.24-7.15 (m, 25H), 5.93-5.81 (m, 1H), 5.26-5.21 (m, 1H), 5.14 (dd, J=10.4, 1.6 Hz, 1H), 4.67-4.65 (m, 2H), 4.64-4.61 (m, 8H), 4.60-4.57 (m, 4H), 3.99-3.93 (m, 2H), 3.75-3.63 (m, 10H), 3.59-3.55 (m, 6H), 3.54-3.46 (m, 20H), 3.21 (d, J=4.8 Hz, 4H).

### Step 4

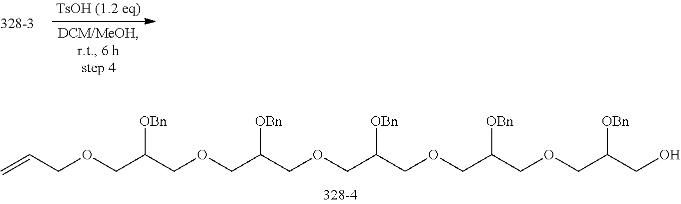 [SMILES: C=CCOCC(COCC(COCC(COCC(COCC(CO)OCc1ccccc1)OCc1ccccc1)OCc1ccccc1)OCc1ccccc1)OCc1ccccc1]

To a solution of 328-3 (3.6 g, 3.213 mmol) in a mixture solvent of DCM (22 mL) and MeOH (11 mL) was added TsOH·H₂O (733 mg, 3.855 mmol), the resulting mixture was stirred for 6 hrs at room temperature until 328-3 was consumed by TLC. The reaction was quenched with saturated NaHCO₃ (aq.), diluted with water (20 mL), and then extracted with DCM (40 mL×3). The organic phase was combined, dried over anhydrous Na₂SO₄, filtered and concentrated to dryness under reduced pressure to give the crude product, which was purified with column chromatography (silica, 0-60% ethyl acetate in petroleum ether) affording 328-4 (2.84 g, 23.233 mmol, 100%) as a colorless oil. Purity=90%-95%. ¹H NMR (400 MHz, CDCl₃) δ 7.38-7.26 (m, 25H), 5.96-5.84 (m, 1H), 5.27 (dq, J=17.2, 1.8 Hz, 1H), 5.17 (dq, J=10.4, 1.6 Hz, 1H), 4.73-4.63 (m, 10H), 4.63-4.58 (m, 1H), 3.99 (dt, J=5.6, 1.6 Hz, 2H), 3.78-3.71 (m, 5H), 3.67-3.63 (m, 2H), 3.62-3.54 (m, 18H).

### Step 5

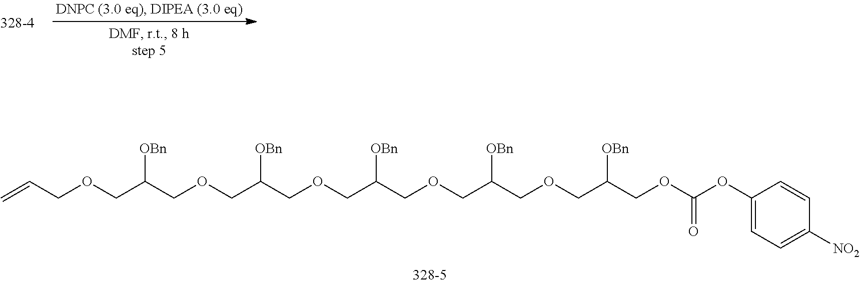 [SMILES: C=CCOCC(COCC(COCC(COCC(COCC(COC(=O)Oc1ccc([N+](=O)[O-])cc1)OCc1ccccc1)OCc1ccccc1)OCc1ccccc1)OCc1ccccc1)OCc1ccccc1]

To a solution of 328-4 (3.6 g, 3.213 mmol) in DMF (8 mL) was added DIPEA (353.2 mg, 2.733 mmol), followed by 4,4′-dinitrodiphenyl carbonate (831.1 mg, 2.732 mmol), then the resulting mixture was stirred at room temperature for 8 hrs until 328-4 was consumed detected by LCMS. The reaction solution was directly used in the next step without work-up procedure.

### Step 6

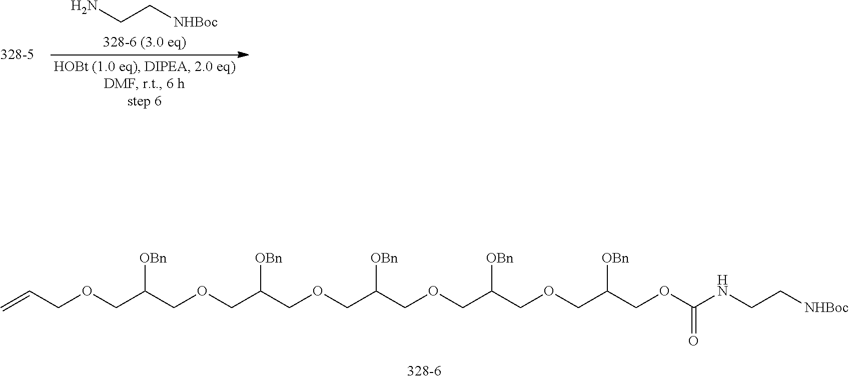 [SMILES: CC(C)(C)OC(=O)NCCN ; C=CCOCC(COCC(COCC(COCC(COCC(COC(=O)NCCNC(=O)OC(C)(C)C)OCc1ccccc1)OCc1ccccc1)OCc1ccccc1)OCc1ccccc1)OCc1ccccc1]

To the above reaction mixture was added HOBt (123 mg, 0.911 mmol), DIPEA (235.5 mg, 1.822 mmol) and 328-6 (437.6 mg, 2.733 mmol) successively, and the resulting mixture was stirred at room temperature for 6 hrs until 328-5 was consumed detected by LCMS. The reaction mixture was diluted with ethyl acetate (100 mL) and washed with saturated NaHCO₃ (aq, 30 mL×3), dried over anhydrous Na₂SO₄, filtered and concentrated to dryness under reduced pressure. The crude product was purified with column chromatography (silica, 0-90% ethyl acetate in petroleum ether) affording 328-6 (958 mg, 0.90 mmol, 98.7% over 2 steps) as a pale yellow oil. H NMR (400 MHz, CDCl₃) δ 7.36-7.22 (m, 25H), 5.95-5.80 (m, 1H), 5.28-5.21 (m, 1H), 5.20-5.13 (m, 1H), 5.07 (s, 1H), 4.83 (s, 1H), 4.69-4.62 (m, 10H), 4.30-4.21 (m, 1H), 4.16-4.10 (m, 1H), 3.97 (dt, J=5.6, 1.6 Hz, 2H), 3.77-3.69 (m, 5H), 3.62-3.50 (m, 19H), 3.27-3.10 (m, 4H), 1.43 (s, 9H).

### Step 7

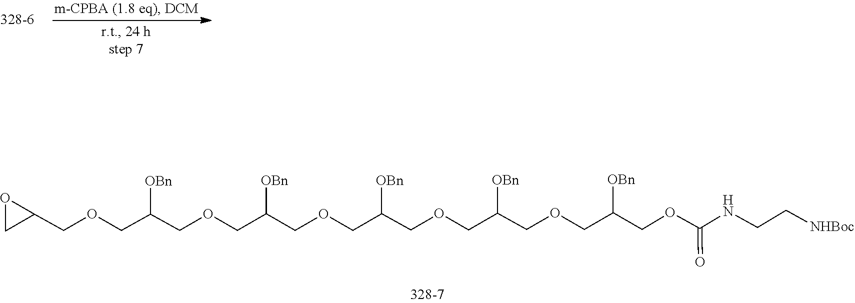 [SMILES: CC(C)(C)OC(=O)NCCNC(=O)OCC(COCC(COCC(COCC(COCC(COCC1CO1)OCc1ccccc1)OCc1ccccc1)OCc1ccccc1)OCc1ccccc1)OCc1ccccc1]

To a solution of 328-6 (958 mg, 0.90 mmol) in DCM (6 mL) was added a solution of m-CPBA (280 mg, 1.62 mmol) in DCM (6 mL) dropwise at room temperature. The resulting mixture was stirred at this temperature for 24 hours until 328-6 was consumed, and the reaction was quenched with saturated Na₂S₂O₃ (aq, 5 mL) and NaHCO₃ (aq., 5 mL). The reaction mixture was stirred for 30 mins, and then diluted with DCM (30 mL). The organic phase was washed with a mixture solution (10 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-100% ethyl acetate in petroleum ether) affording 328-7 (787 mg, 0.728 mmol, 80.9%) as a pale yellow oil. Purity=90%-95%.

### Step 8

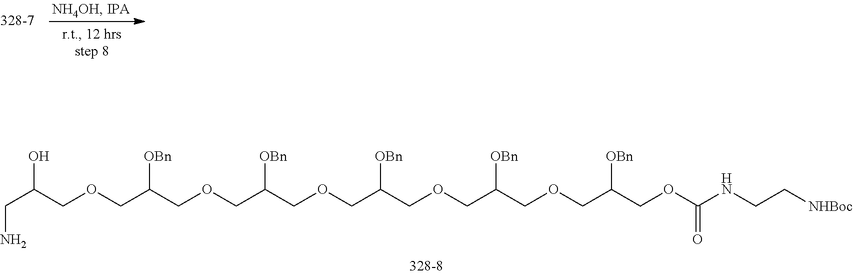 [SMILES: CC(C)(C)OC(=O)NCCNC(=O)OCC(COCC(COCC(COCC(COCC(COCC(O)CN)OCc1ccccc1)OCc1ccccc1)OCc1ccccc1)OCc1ccccc1)OCc1ccccc1]

To a solution of 328-7 (565 mg, 0.523 mmol) in isopropyl alcohol (43 mL) was added ammonia (43 mL) dropwise at room temperature, and the resulting mixture was stirred at this temperature for 12 hours until 328-7 was consumed. The reaction mixture was concentrated to dryness under reduced pressure to afford 328-8 (552.6 mg, 0.503 mmol, 96.3%) as a yellow oil, which was used in the next step without further purification. Purity=90%-95%.

### Step 9

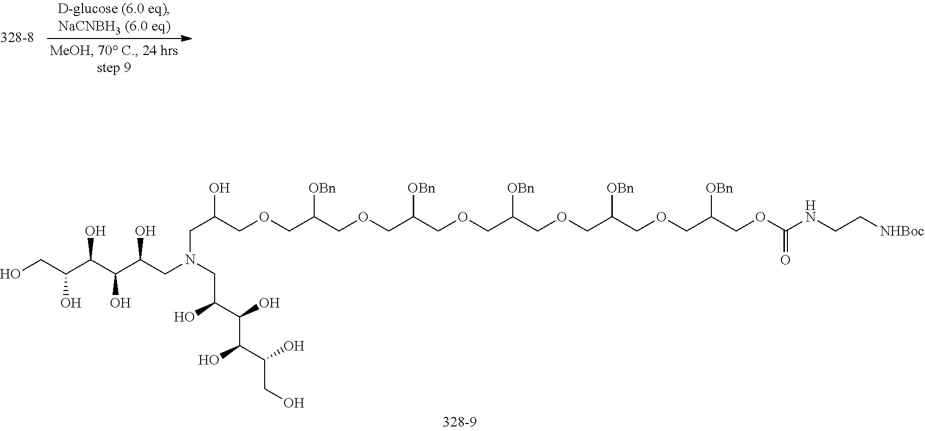 [SMILES: CC(C)(C)OC(=O)NCCNC(=O)OCC(COCC(COCC(COCC(COCC(COCC(O)CN(C[C@H](O)[C@@H](O)[C@H](O)[C@H](O)CO)C[C@H](O)[C@@H](O)[C@H](O)[C@H](O)CO)OCc1ccccc1)OCc1ccccc1)OCc1ccccc1)OCc1ccccc1)OCc1ccccc1]

A mixture of 328-8 (552.6 mg, 0.503 mmol), D-glucose (544.2 mg, 3.021 mmol) and NaCNBH₃ (189.8 mg, 3.021 mmol) in anhydrous MeOH (12 mL) was stirred at 70° C. for 24 hrs until most of 328-8 was consumed and 328-9 was detected by LCMS. The reaction mixture was cooled down to room temperature, filtered and concentrated under reduced pressure to give the crude product, which was purified by reverse phase liquid chromatography to give 328-9 (597 mg, 0.419 mmol, 83.2%) as a colorless oil. Purity=90%-95%. ¹H NMR (400 MHz, MeOH-d₄) δ 7.41-7.20 (m, 25H), 4.69-4.57 (m, 10H), 4.26-4.04 (m, 5H), 3.84-3.78 (m, 3H), 3.77-3.68 (m, 8H), 3.67-3.42 (m, 28H), 3.38-3.34 (m, 1H), 3.33-3.31 (m, 1H), 3.17-3.06 (m, 4H), 1.40 (s, 9H).

### Step 10

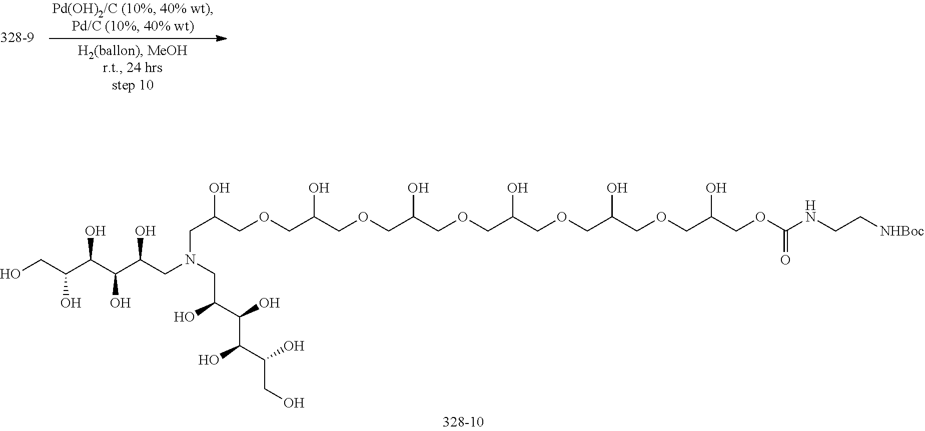 [SMILES: CC(C)(C)OC(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

A mixture of 328-9 (376 mg, 0.264 mmol), Pd(OH)₂/C (10%, 150 mg) and Pd/C (10%, 150 mg) in MeOH (30 mL) was stirred under hydrogen atmosphere (ballon) for 24 h at room temperature until 328-9 was completely converted into 328-10. The reaction mixture was filtered through a celite pad, and the filtrate was concentrated to dryness under reduced pressure to afford 328-10 (211.4 mg, 0.217 mmol, 82.1%) as an off-white solid, which was used in the next step without further purification.

### Step 11

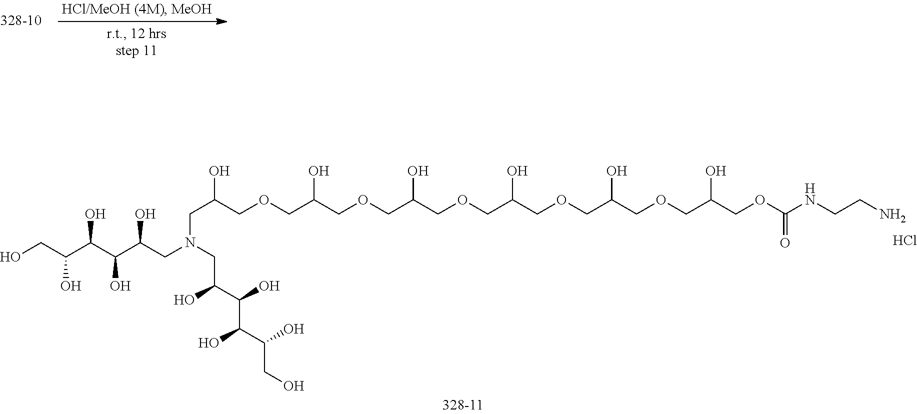 [SMILES: NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO ; Cl]

A solution of 328-10 (211.4 mg, 0.216 mmol) in HCl/MeOH (4M, 3 mL) and MeOH (3 mL) was stirred at room temperature for 12 hours until 328-10 was completely converted into 328-11. The reaction mixture was concentrated to dryness under reduced pressure to afford 328-11 (198.0 mg, 0.217 mmol, 100%) as an off-white solid, which was used in the next step without further purification.

### Step 12

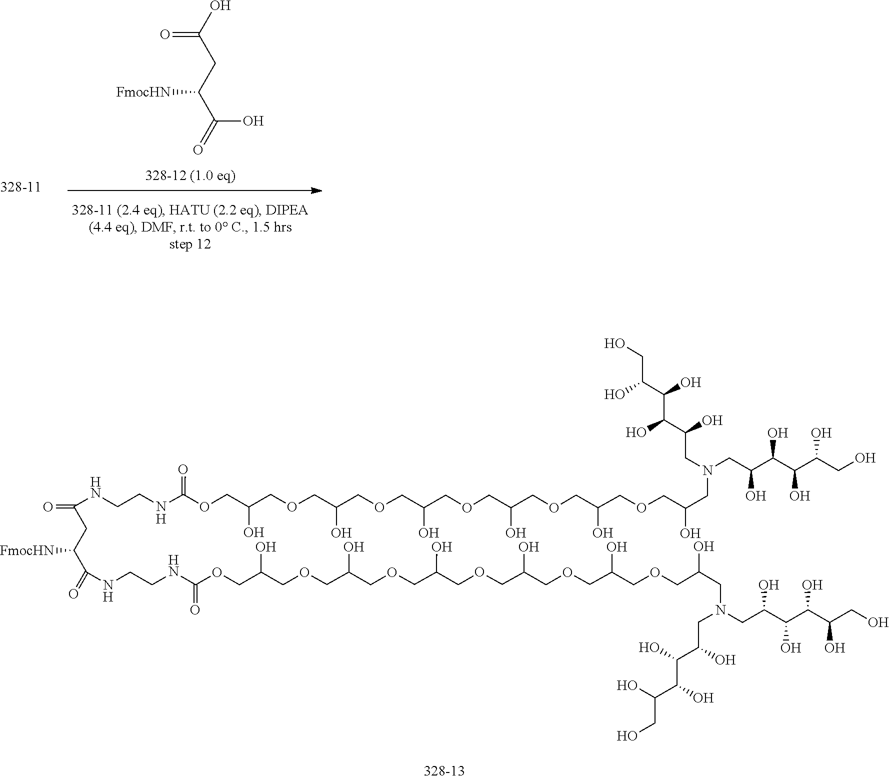

A solution of 328-12 (24.8 mg, 0.0698 mmol) and HATU (58.4 mg, 0.153 mmol) in anhydrous DMF (2 mL) was stirred at room temperature for 15 mins, then it was stirred in an ice bath. A solution of 328-11 (150 mg, 0.168 mmol) in anhydrous DMF (2 mL) was added dropwise, followed by DIPEA (39.7 mg, 0.307 mmol). The resulting mixture was stirred in the ice bath for 1 h until most of 328-12 was consumed. The reaction mixture was purified by reverse phase liquid chromatography to give 328-13 (97.5 mg, 0.0471 mmol, 67.4%) as a colorless oil. Purity=90%-95%.

### Step 13

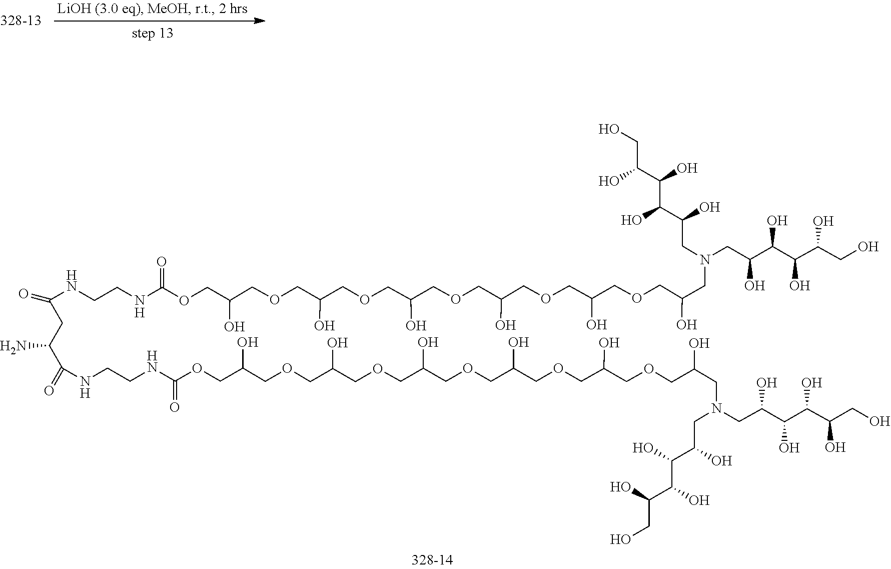 [SMILES: N[C@H](CC(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

To a solution of 328-13 (97.5 mg, 0.0471 mmol) in MeOH (3 mL) was added LiOH·H₂O (5.9 mg, 0.141 mmol), and the mixture was stirred at room temperature for 2 hrs until 328-13 was consumed. The reaction solution was neutralized with iN HCl to pH=7, and concentrated under reduced pressure to give a crude product, which was dissolved in H₂O (10 mL) and washed with hexane (5 mL×3). The aqueous phase was concentrated to dryness under reduced pressure to afford 328-14 (87.0 mg, 0.047 mmol, 100%) as a colorless oil, which was used in the next step without further purification.

### Example 20: Preparation of an Exemplary Polar Group

### Step 1

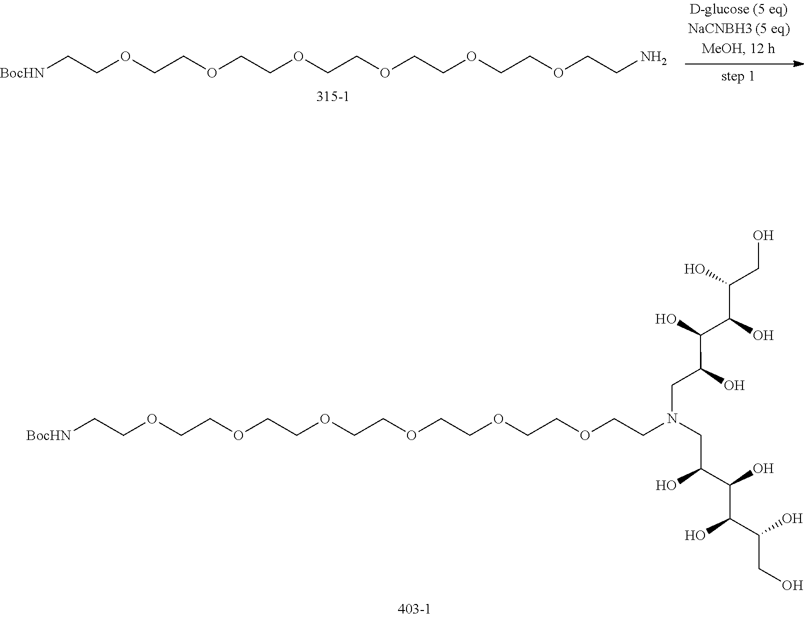 [SMILES: CC(C)(C)OC(=O)NCCOCCOCCOCCOCCOCCOCCN ; CC(C)(C)OC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 315-1 (1.0 g, 2.355 mmol) and D-Glucose (2.1 g, 11.656 mmol) in anhydrous Methanol (40 mL) was heated at 50° C. for 30 min, then NaCNBH₃ (740 mg, 11.776 mmol) was added. The resulting solution was stirred for another 12 hr at 70° C. until indicated all starting amine was disappeared and the mass of desired product was detected. Then the reaction solution was concentrated and purified by reverse phase liquid chromatography to give 403-1 (730 mg, 0.970 mmol, 41.17%) as a white solid.

### Step 2

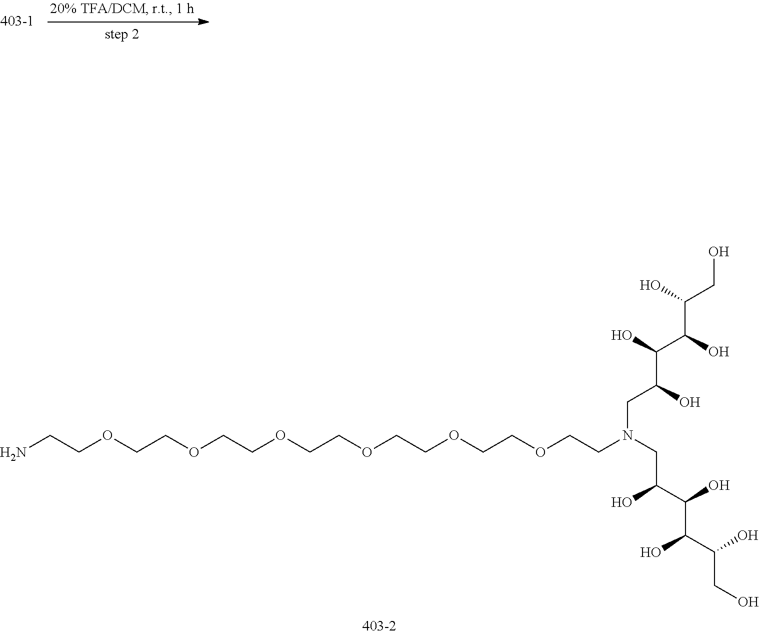 [SMILES: NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 403-1 (730 mg, 0.970 mmol) and TFA (2 mL) in anhydrous DCM (8 mL) was stirred at room temperature for 1 hr until LCMS indicated all starting amine was disappeared and desired product was detected. Then the solution was concentrated to dryness to 403-2 (610 mg, 0.935 mmol, 96.39%) as yellow oil, used as such in the next step.

### Step 3

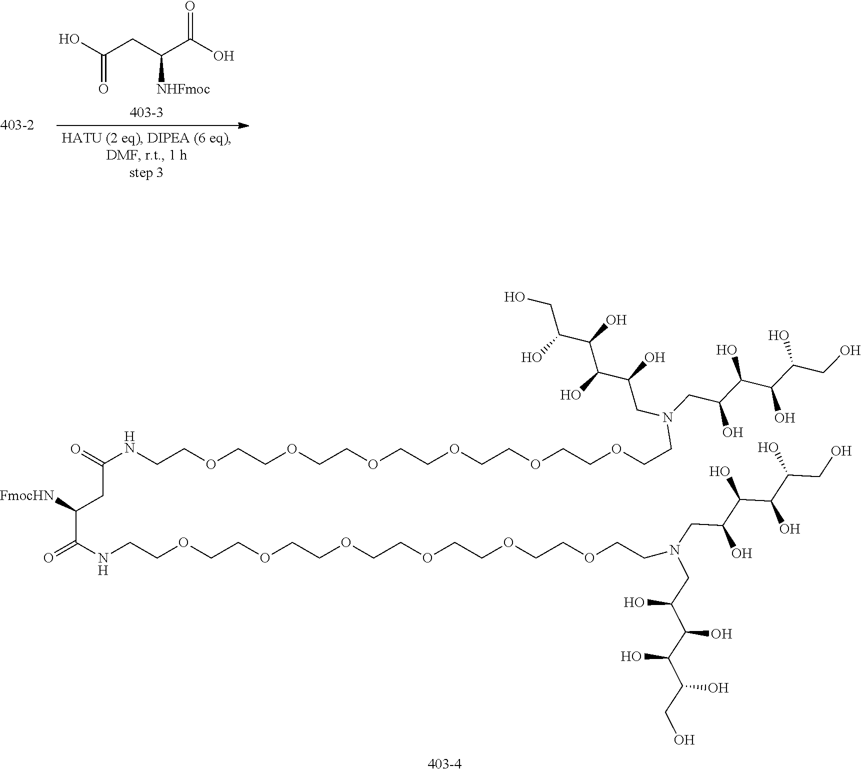

A solution of 403-2 (610 mg, 0.935 mmol), 403-3 (166 mg, 0.467 mmol) and HATU (355 mg, 0.934 mmol) in anhydrous DMF (5 mL) was stirred at room temperature for 5 min, then DIPEA (362 mg, 2.801 mmol) was added. The resulting solution was stirred for another 1 hr at r.t. until LCMS indicated complete reaction. The reaction solution was purified directly by reverse phase liquid chromatography to give 403-4 (410 mg, 0.252 mmol, 54.03%) as a white solid.

### Step 4

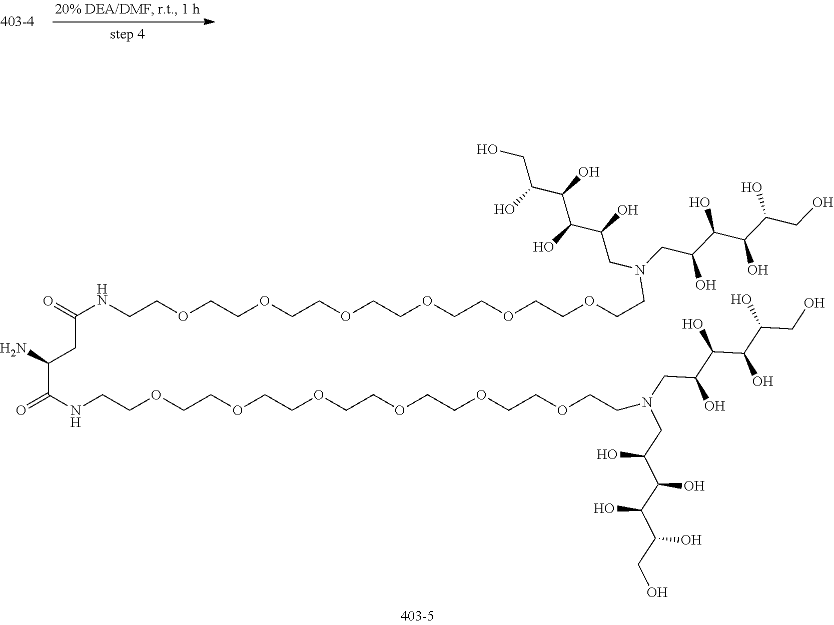 [SMILES: N[C@@H](CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 403-4 (200 mg, 0.123 mmol) and DEA (1 mL) in anhydrous DMF (4 mL) was stirred at room temperature for 1 hr until LCMS indicated all starting amine was disappeared and desired product was detected. Then the solution was concentrated to dryness to 403-5 (167 mg, 0.119 mmol, 96.80%) as colorless oil, used as such in the next step.

### Example 21: Preparation of an Exemplary Polar Group

### Step 1

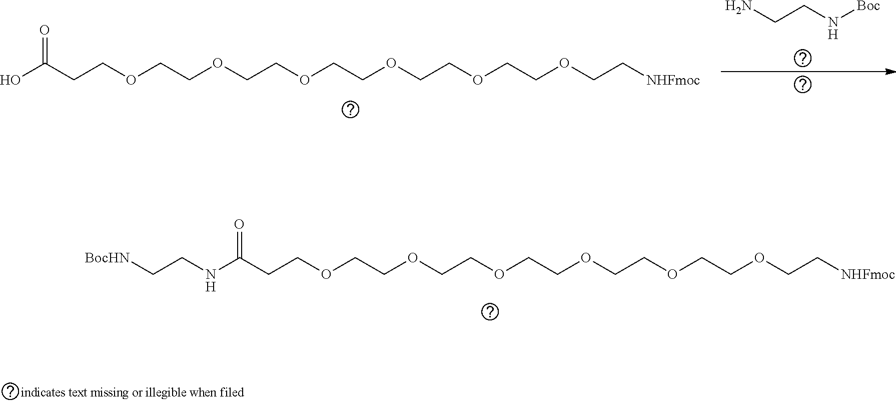

A solution of 341-1 (1 g, 1.737 mmol), 311-2 (278 mg, 1.735 mmol) and HATU (661 mg, 1.738 mmol) in anhydrous DMF (10 mL) was stirred at room temperature for 5 min, then DIPEA (674 mg, 5.215 mmol) was added. The resulting solution was stirred for another 1 hr at r.t. until LCMS indicated complete reaction. The reaction solution was purified directly by reverse phase liquid chromatography to give 341-2 (950 mg, 1.323 mmol, 76.17%) as a white solid. purity=90%-95%.

### Step 2

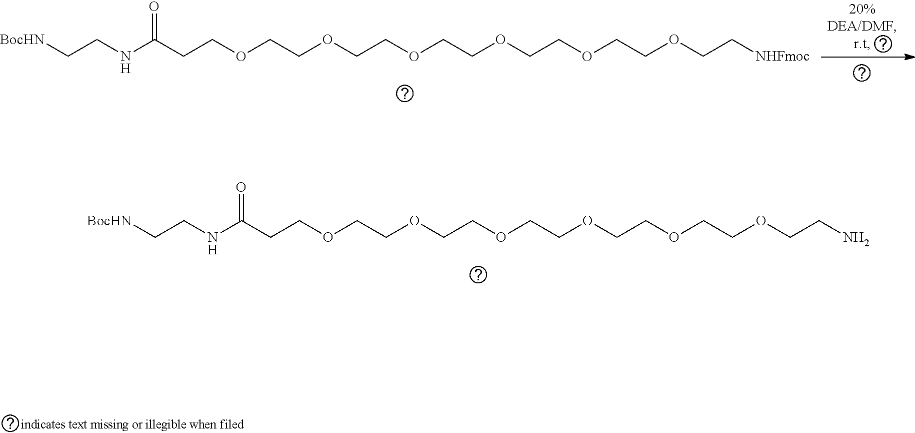

A solution of 341-2 (950 mg, 1.323 mmol) and DEA (2 mL) in anhydrous DMF (8 mL) was stirred at room temperature for 1 hr until LCMS indicated all starting amine was disappeared and desired product was detected. Then the solution was concentrated to dryness to 341-3 (610 mg, 1.231 mmol, 93.05%) as colorless oil, used as such in the next step. purity=90%-95%.

### Step 3

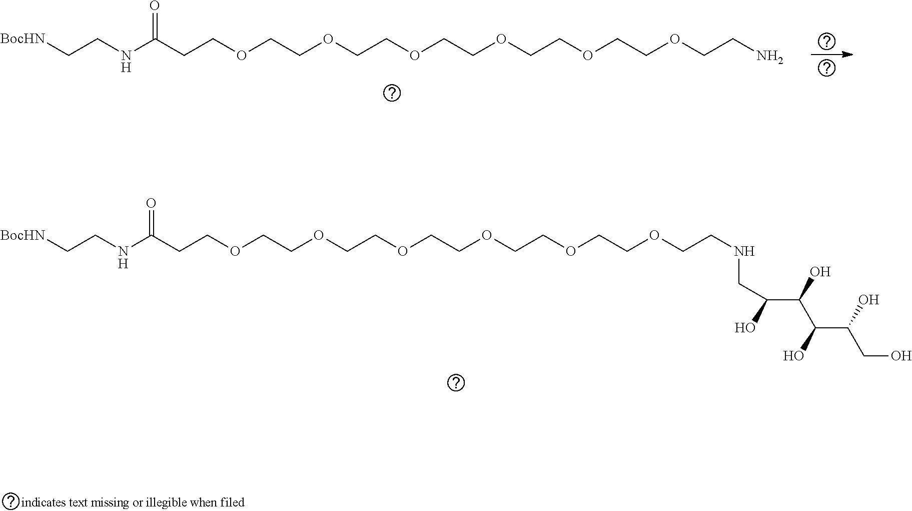 [SMILES: CC(C)(C)OC(=O)NCCNC(=O)CCOCCOCCOCCOCCOCCOCCN ; CC(C)(C)OC(=O)NCCNC(=O)CCOCCOCCOCCOCCOCCOCCNC[C@H](O)[C@@H](O)[C@H](O)[C@H](O)CO]

A solution of 341-3 (610 mg, 1.231 mmol) and D-Glucose (444 mg, 2.464 mmol) in anhydrous Methanol (50 mL) was heated at 50° C. for 30 min, then NaCNBH₃ (155 mg, 2.467 mmol) was added. The resulting solution was stirred for another 2 hr at 70° C. until indicated all starting amine was disappeared and the mass of desired product was detected. Then the reaction solution was concentrated and purified by reverse phase liquid chromatography to give 341-4 (340 mg, 0.515 mmol, 41.84%) as a white solid. purity=90%-95%.

### Step 4

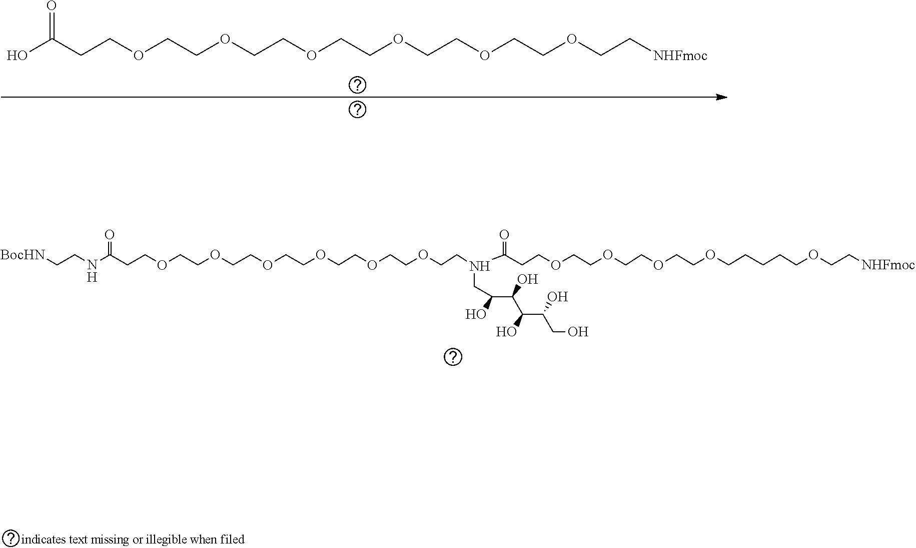

A solution of 341-4 (340 mg, 0.515 mmol), 341-1(297 mg, 0.516 mmol) and HATU (196 mg, 0.515 mmol) in anhydrous DMF (10 mL) was stirred at room temperature for 5 min, then DIPEA (200 mg, 1.548 mmol) was added. The resulting solution was stirred for another 1 hr at r.t. until LCMS indicated complete reaction. The reaction solution was purified directly by reverse phase liquid chromatography to give 341-5 (475 mg, 0.390 mmol, 75.73%) as a white solid. purity=90%-95%.

### Step 5

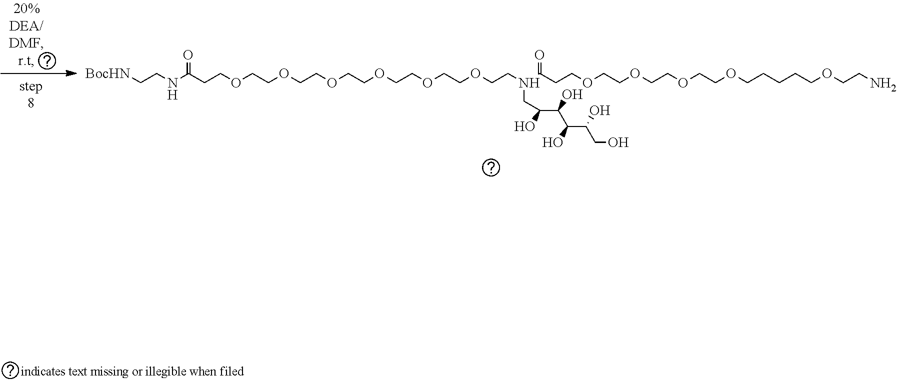 [SMILES: CC(C)(C)OC(=O)NCCNC(=O)CCOCCOCCOCCOCCOCCOCCN(C[C@H](O)[C@@H](O)[C@H](O)[C@H](O)CO)C(=O)CCOCCOCCOCCOCCCCCOCCN]

A solution of 341-5 (475 mg, 0.390 mmol) and DEA (2 mL) in anhydrous DMF (8 mL) was stirred at room temperature for 1 hr until LCMS indicated all starting amine was disappeared and desired product was detected. Then the solution was concentrated to dryness to 341-6 (372 mg, 0.374 mmol, 95.90%) as colorless oil, used as such in the next step. purity=90%-95%.

### Step 6

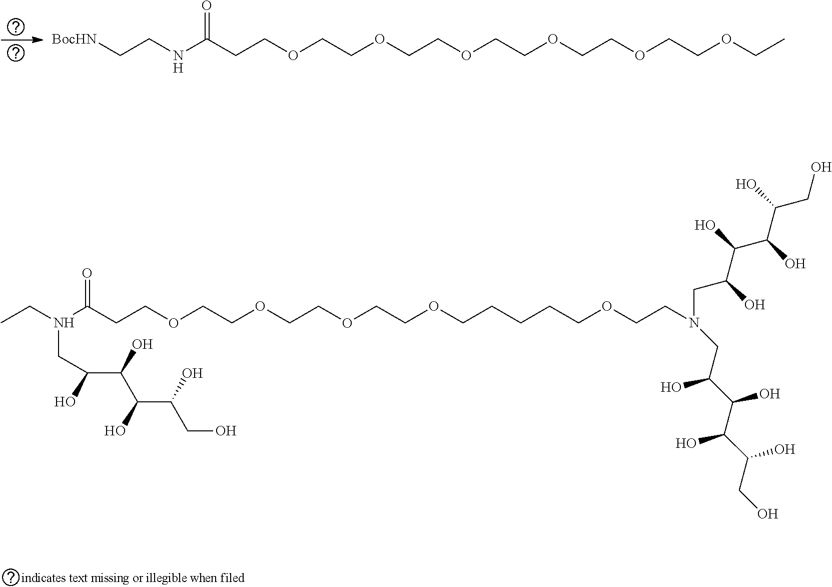

A solution of 341-6 (372 mg, 0.374 mmol) and D-Glucose (337 mg, 1.871 mmol) in anhydrous Methanol (50 mL) was heated at 50° C. for 30 mi, then NaCNBH₃ (118 mg, 1.878 mmol) was added. The resulting solution was stirred for another 6 hr at 70° C. until indicated all starting amine was disappeared and the mass of desired product was detected. Then the reaction solution was concentrated and purified by reverse phase liquid chromatography to give 341-7 (406 mg, 0.307 mmol, 82.09%) as a white solid. purity=90%-95%.

### Step 7

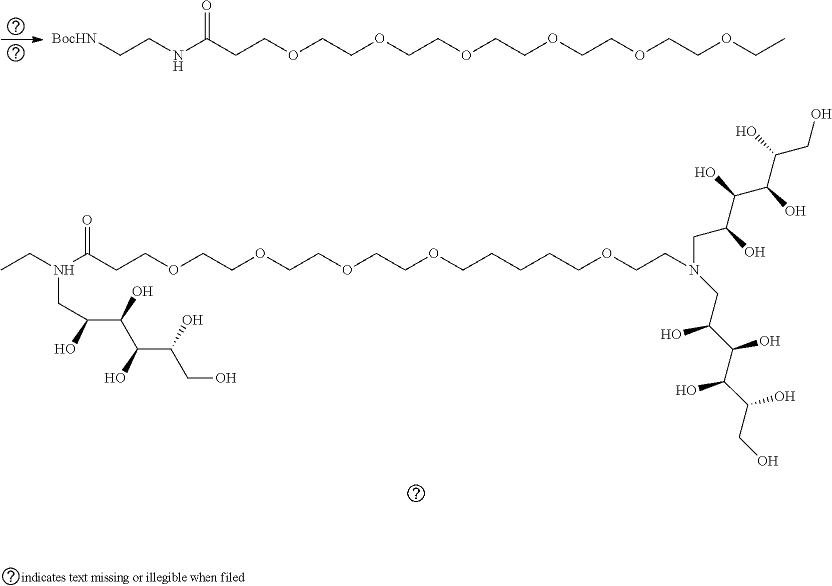

A solution of 341-7 (100 mg, 0.076 mmol) and TFA (2 mL) in anhydrous DCM (8 mL) was stirred at room temperature for 1 hr until LCMS indicated all starting amine was disappeared and desired product was detected. Then the solution was concentrated to dryness to 341-8 (85 mg, 0.069 mmol, 90.79%) as yellow oil, used as such in the next step. purity=90%-95%.

### Example 22: Preparation of a Drug-Linker Attached to Exatecan for Further Attachment to a Polar Group

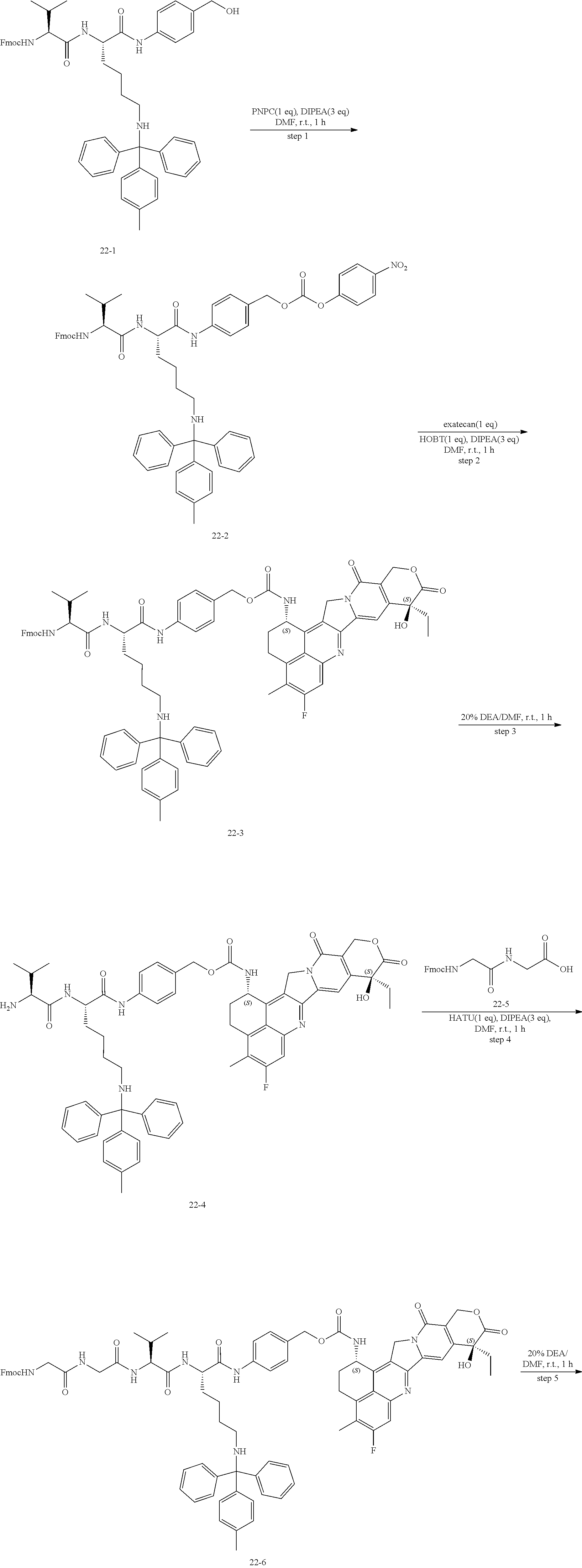

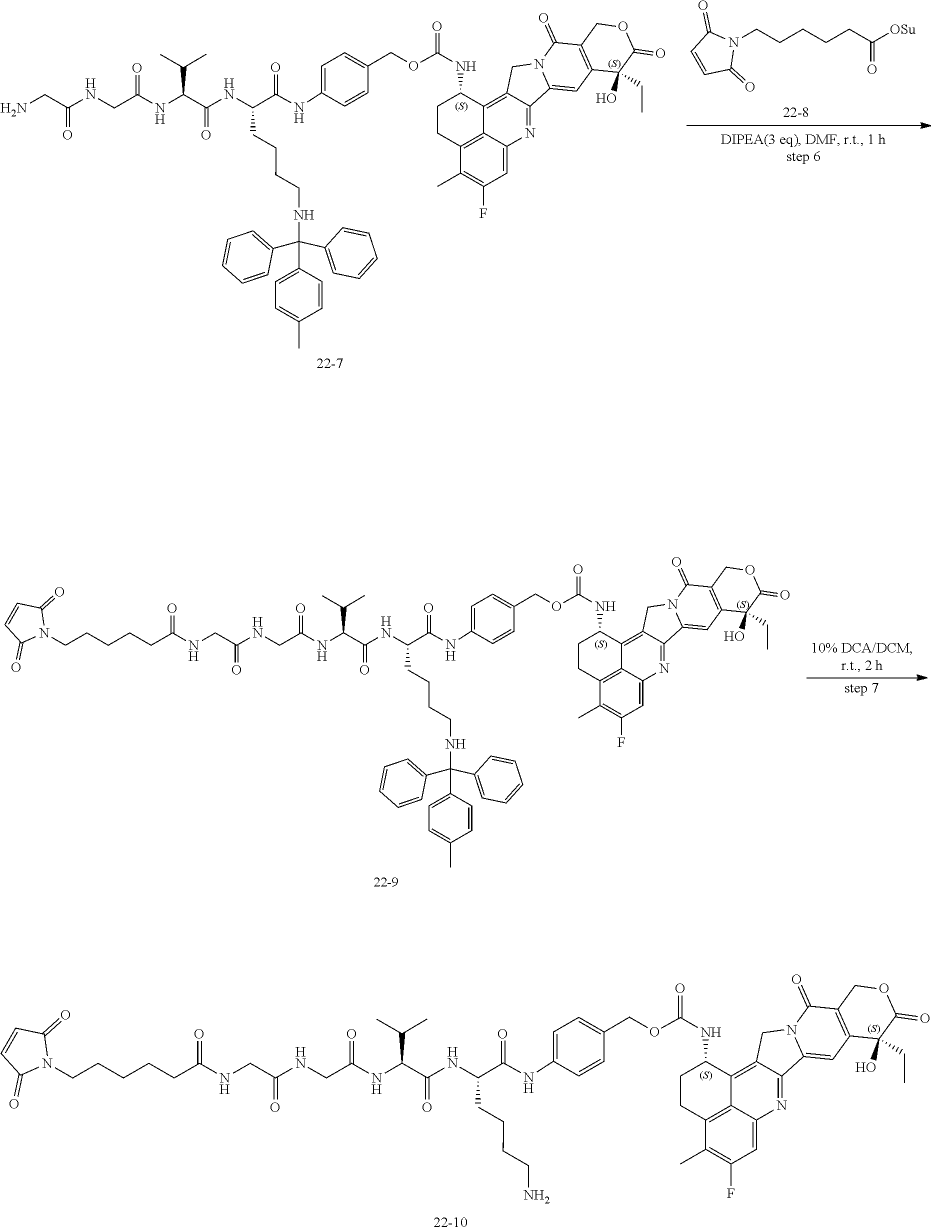

A Drug-Linker attached to exatecan for further attachment to a Polar Group is prepared as follows:

### Step 1

A solution of compound 22-1 and DIPEA in anhydrous DMF is stirred at room temperature for 5 min, then PNPC is added. The resulting solution is stirred for another 1 hr at r.t. until LCMS indicates all starting amine is consumed and the desired product is detected. Then most of the DMF and DIPEA is evaporated and the residue is washed in acetonitrile at 5° C. for 30 minutes. The solution is filtered and the filter cake is washed with acetonitrile to yield compound 22-2.

### Step 2

A solution of compound 22-2, exatecan and HOBT in anhydrous DMF is stirred at room temperature, then DIPEA is added. The resulting solution is stirred for another 1 hr at r.t. until LCMS indicates all starting amine is consumed and the desired product is detected. Then most of the DMF and DIPEA is evaporated and the residue is washed in acetonitrile at 5° C. for 30 minutes. The solution is filtered and the filter cake is washed with acetonitrile to yield compound 22-3.

### Step 3

A solution of compound 22-3 and DEA in anhydrous DMF is stirred at room temperature for 1 hr until LCMS indicated all starting amine is consumed and the desired product is detected. Then most of the DMF and DEA is evaporated and the residue is washed in acetonitrile at 5° C. for 30 minutes. The solution is filtered and the filter cake is washed with acetonitrile to yield compound 22-4.

### Step 4

A solution of compound 22-4, compound 22-5 and HATU in anhydrous DMF is stirred at room temperature for 5 min, then DIPEA is added. The resulting solution is stirred for another 1 hr at r.t. until LCMS indicates complete reaction. Then most of the DMF and DIPEA is evaporated and the residue is washed in acetonitrile at 5° C. for 30 minutes. The solution is filtered and the filter cake is washed with acetonitrile to yield compound 22-6.

### Step 5

A solution of compound 22-6 and DEA in anhydrous DMF is stirred at room temperature for 1 hr until LCMS indicates all starting amine is consumed and the desired product is detected. Then most of the DMF and DEA is evaporated and the residue is washed in acetonitrile at 5° C. for 30 minutes. The solution is filtered and the filter cake is washed with acetonitrile to yield compound 22-7.

### Step 6

A solution of compound 22-7 and DIPEA in anhydrous DMF is stirred at room temperature for 5 min, then a solution of compound 22-8 in anhydrous DMF is added dropwise by syringe over 2 min. The resulting solution is stirred for another 1 hr at r.t. until LCMS indicates all starting amine is consumed and the mass of desired product is detected. Then most of the DMF and DIPEA is evaporated and the residue is washed in acetonitrile at 5° C. for 30 minutes. The solution is filtered and the filter cake is washed with acetonitrile to yield compound 22-9.

### Step 7

A solution of compound 22-9 and DCA in anhydrous DCM is stirred at room temperature for 2 hr until LCMS indicates all starting amine is consumed and the desired product is detected. The reaction solution is concentrated to dryness and purified directly by reverse phase liquid chromatography to give 22-10.

### Example 23: Preparation of an Exemplary Linker Drug

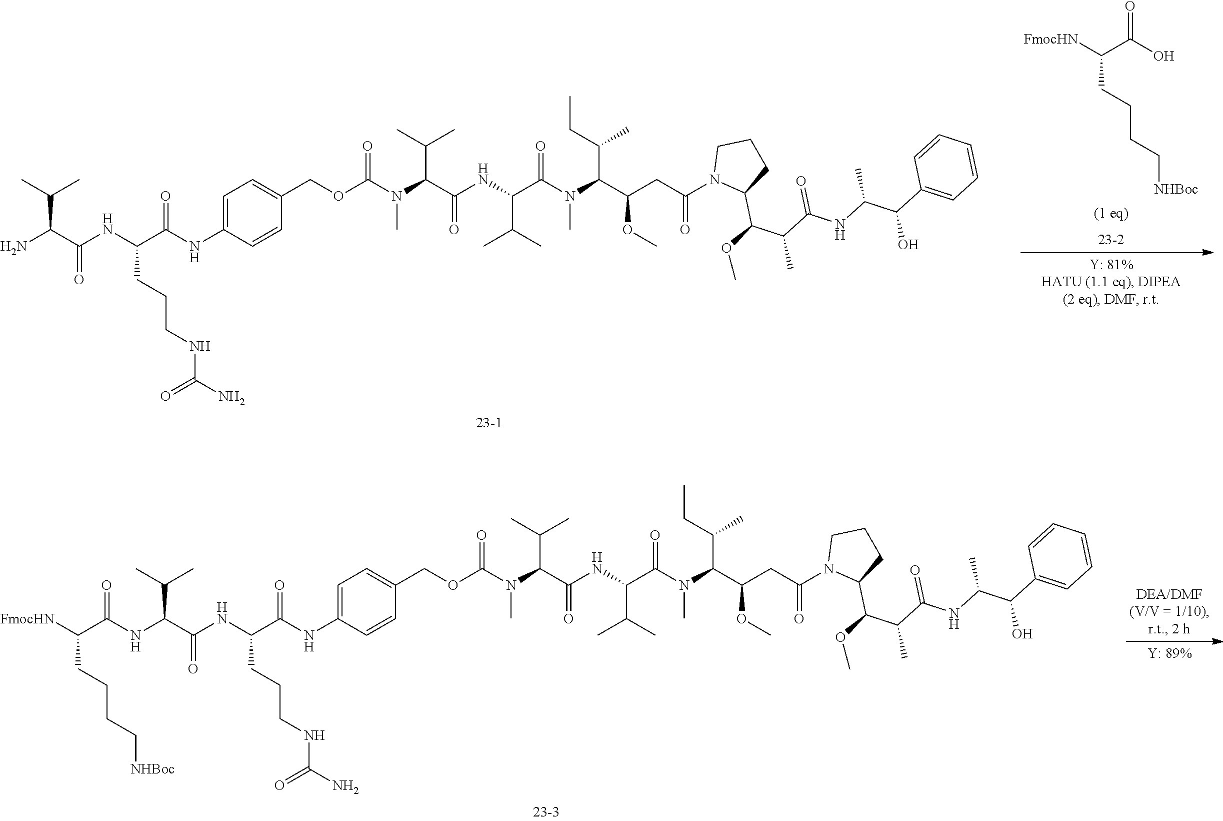

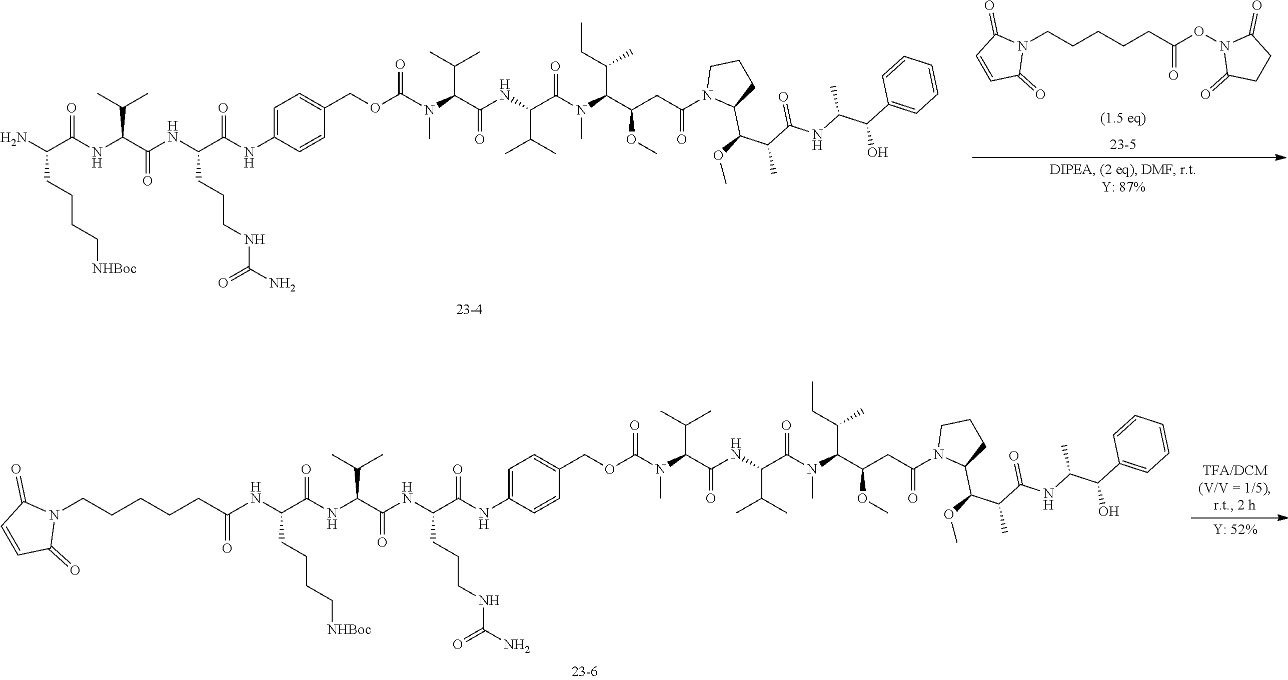

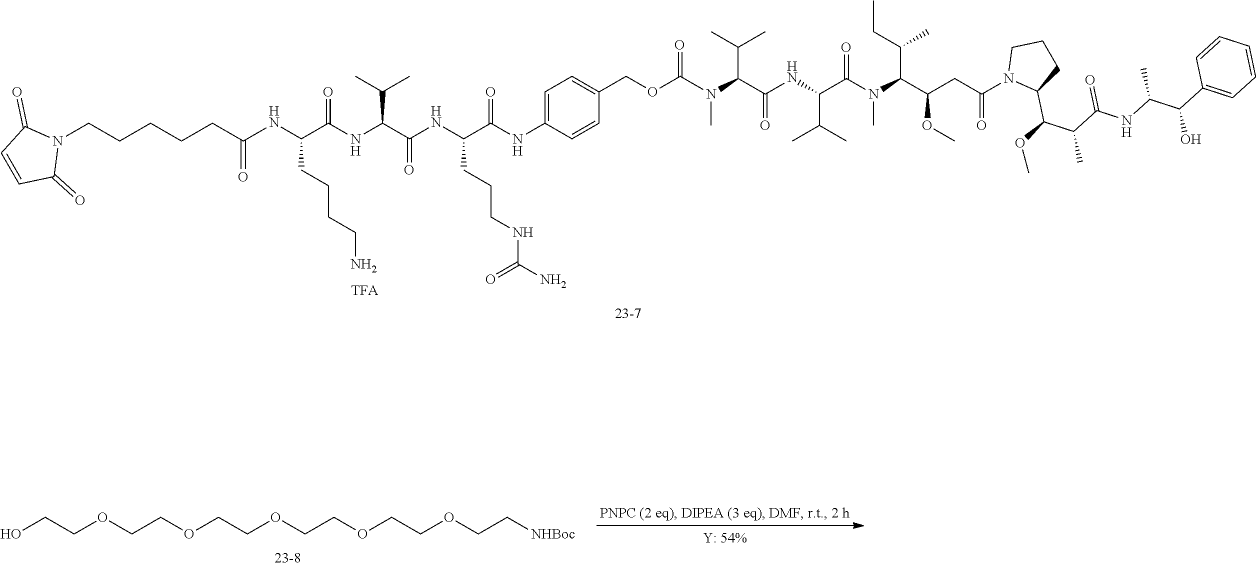 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCCN)NC(=O)CCCCCN2C(=O)C=CC2=O)C(C)C)cc1)C(C)C ; CC(C)(C)OC(=O)NCCOCCOCCOCCOCCOCCO]

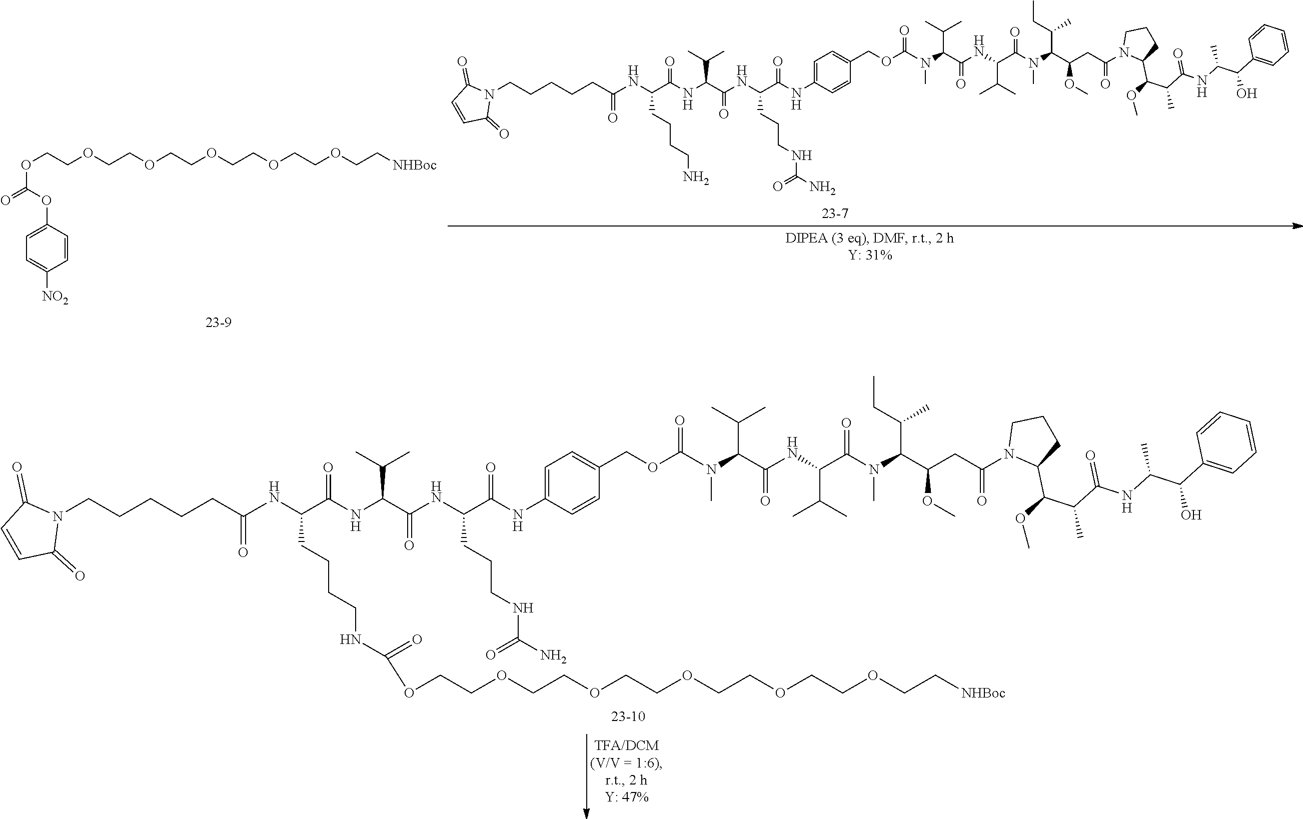

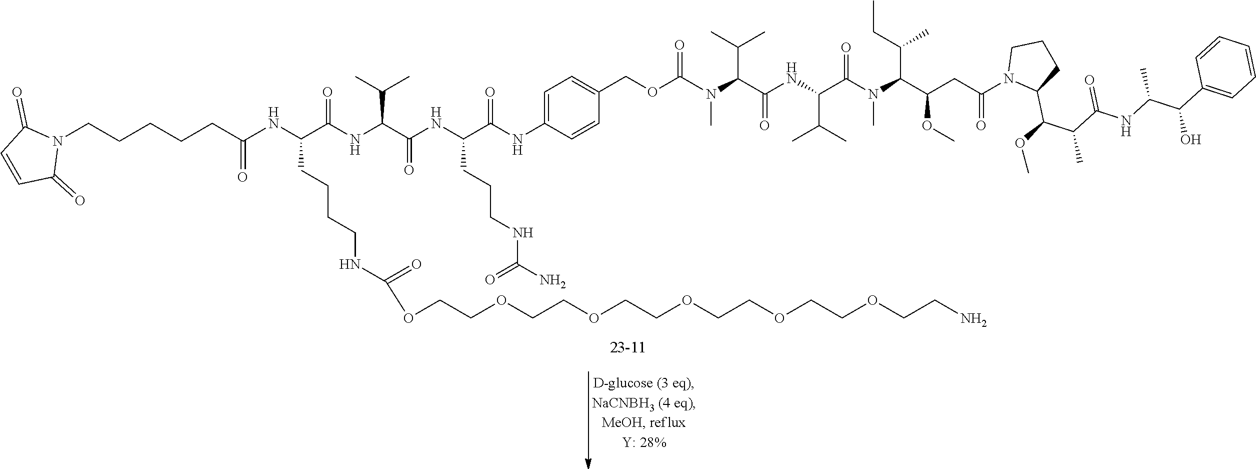 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCCNC(=O)OCCOCCOCCOCCOCCOCCN)NC(=O)CCCCCN2C(=O)C=CC2=O)C(C)C)cc1)C(C)C]

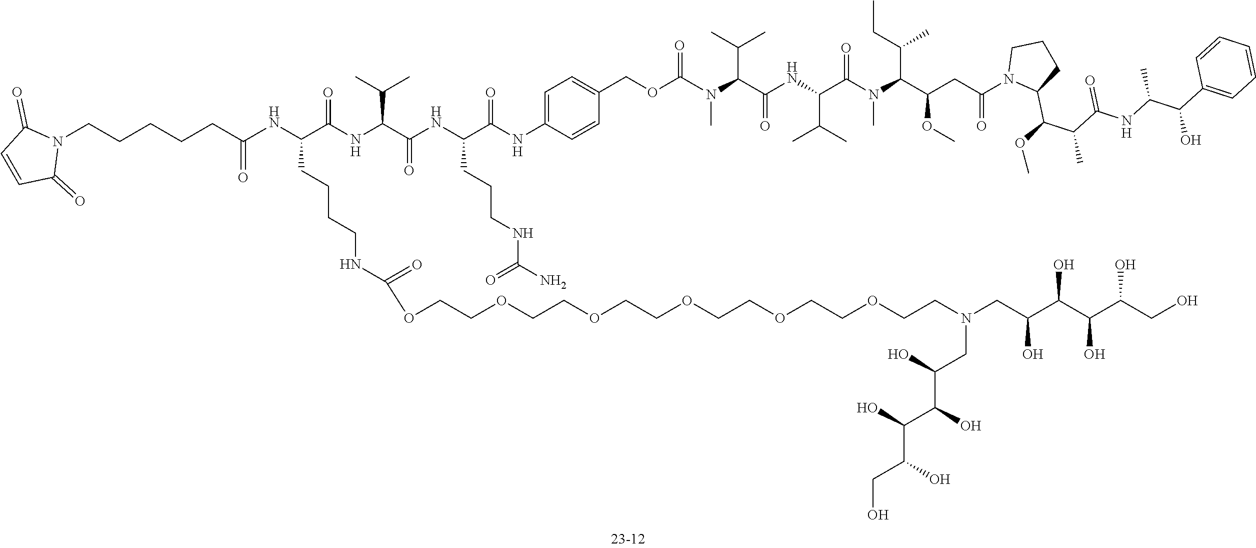 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCCNC(=O)OCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN2C(=O)C=CC2=O)C(C)C)cc1)C(C)C]

An Exemplary Linker Drug was prepared as follows:

### Step 1

A solution of 4-((S)-2-((S)-2-amino-3-methylbutanamido)-5-ureidopentanamido)benzyl (S)-1-((S)-1-(((3S,4S,5S)-1-((S)-2-((1R,2R)-3-((1R,2S)-1-hydroxy-1-phenylpropan-2-ylamino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-ylamino)-3-methyl-1-oxobutan-2-yl(methyl)carbamate (23-1) (1.058 g, 0.942 mmol), (S)-2-(((9H-fluoren-9-yl)methoxy)carbonylamino)-6-(tert-butoxycarbonylamino)hexanoic acid (23-2) (0.44 g, 0.942 mmol) and HATU (0.39 g, 1.036 mmol), DIPEA (0.24 g, 1.884 mmol) in DMF (10 mL) was stirred at room temperature for 2 h until LCMS showed that the reaction was completed. The reaction mixture was purified directly by reverse phase column chromatography (C18 column, eluting with 0-70% acetonitrile in water with 0.01% TFA) to get desired fractions, which was freeze-dried to get tert-butyl N-[(5S)-5-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl) carbamoyl}-2-methylpropyl]carbamoyl}-2-methylpropyl](methyl)carbamoyl}oxy)methyl]phenyl}carbamoyl) butyl]carbamoyl}-2-methylpropyl]carbamoyl}-5-{[(9H-fluoren-9-ylmethoxy)carbonyl]amino}pentyl]carbamate (23-3) (1.2 g, 0.763 mmol, 81.01%) as a white solid. ESI m/z=787.5 (M/2+H)+.

### Step 2

To a solution of compound tert-butyl N-[(5S)-5-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}-5-({[(9H-fluoren-9-yl)methoxy]carbonyl}amino)pentyl]carbamate (23-3) (1.20 g, 0.762 mmol) in DMF (10 mL) was added diethylamine (DEA, 1.0 mL, 12.756 mmol). The mixture was stirred at room temperature for 2 hours to complete. And the solution was concentrated to dryness and then purified by reverse phase column chromatography (C18 column, eluting with 0-100% acetonitrile in water with 0.01% TFA) to get {4-[(2S)-2-[(2S)-2-[(2S)-2-amino-6-{[(tert-butoxy)carbonyl]amino}hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (23-4) (920 mg, 0.681 mmol, 89.32%) as a white solid. ESI m/z=1351.8 (M+H)+.

### Step 3

A solution of {4-[(2S)-2-[(2S)-2-[(2S)-2-amino-6-{[(tert-butoxy)carbonyl]amino}hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (23-4) (920 mg, 0.681 mmol), 2,5-dioxopyrrolidin-1-yl 6-(2,5-dioxo-2,5-dihydro-1H-pyrrol-1-yl)hexanoate (23-5) (314.74 mg, 1.021 mmol) and DIPEA (175.86 mg, 1.361 mmol) in DMF (10 mL) was stirred at room temperature for 2 hours until LCMS showed that the reaction was completed. Then the reaction solution was purified directly by reverse phase column chromatography (C18 column, eluting with 0-70% acetonitrile in water with 0.01% TFA) to get desired fractions, which was freeze-dried to get tert-butyl N-[(5S)-5-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1- hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}-5-[6-(2,5-dioxo-2,5-dihydro-1H-pyrrol-1-yl)hexanamido]pentyl]carbamate (23-6) (920 mg, 0.596 mmol, 87.49%) as a white solid. ESI m/z=772.8 (M/2+H)+.

### Step 4

To a solution of tert-butyl N-[(5S)-5-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}-5-[6-(2,5-dioxo-2,5-dihydro-1H-pyrrol-1-yl)hexanamido]pentyl]carbamate (23-6) (920 mg, 0.596 mmol) in DCM (10 mL) was added TFA (2.0 mL, 26.925 mmol). The mixture was stirred at room temperature for 2 hours to achieve complete deprotection. Then the solution was concentrated to dryness and the residue was purified by reverse phase column chromatography (C18 column, eluting with 0-70% acetonitrile in water with 0.01% TFA) to get TFA salt of {4-[(2S)-2-[(2S)-2-[(2S)-6-amino-2-[6-(2,5-dioxo-2,5-dihydro-1H-pyrrol-1-yl)hexanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (23-7) (MC-KVC-pab-MMAE, 450 mg, 0.311 mmol, 52.30%) as a white solid. ESI m/z=722.8 (M/2+H), 1467.8 (M+Na)+.

### Step 5

A solution of tert-butyl N-(17-hydroxy-3,6,9,12,15-pentaoxaheptadecan-1-yl)carbamate (23-8) (90 mg, 0.236 mmol) in DMF (5 mL) was treated with DIPEA (0.117 mL, 0.708 mmol) and Bis(nitrophenyl)carbonate (PNPC, 143.54 mg, 0.472 mmol). The resulting yellow mixture was stirred at 25° C. for 2 h under Ar. Then the complete reaction solution was purified by reverse phase flash chromatography (C18 column, 0-50% acetonitrile in water with 01% TFA) to give 1-[(19-{[(tert-butoxy)carbonyl]amino}-2,5,8,11,14,17-hexaoxanonadecanoyl)oxy]-4-nitrobenzene (23-9) (70 mg, 0.128 mmol, 54.28%) as a white solid. ESI m/z=no mass.

### Step 6

To a solution of 1-[(19-{[(tert-butoxy)carbonyl]amino}-2,5,8,11,14,17-hexaoxanonadecanoyl)oxy]-4-nitrobenzene (23-9) (75 mg, 0.137 mmol) in DMF (4 mL) was added DIPEA (0.068 mL, 0.412 mmol) and {4-[(2S)-2-[(2S)-2-[(2S)-6-amino-2-[6-(2,5-dioxo-2,5-dihydro-1H-pyrrol-1-yl)hexanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (23-7) (198.26 mg, 0.137 mmol). The reaction mixture was stirred at 25° C. for 2 h under Ar. Then the reaction mixture was purified by reverse phase flash chromatography (C18 column, 0-50% acetonitrile in water with 01% TFA) give title 17-{[(tert-butoxy)carbonyl]amino}-3,6,9,12,15-pentaoxaheptadecan-1-yl N-[(5S)-5-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy) methyl]phenyl}carbamoyl)butyl]carbamoyl}-2-methylpropyl]carbamoyl}-5-[6-(2,5-dioxo-2,5-dihydro-1H-pyrrol-1-yl)hexanamido]pentyl]carbamate (23-10) (80 mg, 0.043 mmol, 31.47%) as a white solid. ESI m/z=926.3 (M/2+H)+.

### Step 7

A solution of 17-{[(tert-butoxy)carbonyl]amino}-3,6,9,12,15-pentaoxaheptadecan-1-yl N-[(5S)-5-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}-5-[6-(2,5-dioxo-2,5-dihydro-1H-pyrrol-1-yl)hexanamido]pentyl]carbamate (23-10) (80 mg, 0.043 mmol) in DCM (3 mL) and TFA (0.5 mL) was stirred at 25° C. for 2 h to complete. The solution was concentrated under vacuo and the residue was purified by reverse phase flash chromatography (C18 column, 0-60% acetonitrile in water with 0.01% TFA) to give 17-amino-3,6,9,12,15-pentaoxaheptadecan-1-yl N-[(5S)-5-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}-5-[6-(2,5-dioxo-2,5-dihydro-1H-pyrrol-1-yl)hexanamido]pentyl]carbamate (23-11) (35 mg, 0.020 mmol, 46.25%) as a white solid. ESI m/z=877.1 (M/2+H)+.

### Step 8

To a solution of 4-((25S,28S,31S)-1-amino-25-(6-(2,5-dioxo-2,5-dihydro-1H-pyrrol-1-yl)hexanamido)-28-isopropyl-19,26,29-trioxo-31-(3-ureidopropyl)-3,6,9,12,15,18-hexaoxa-20,27,30-triazadotriacontan-32-amido)benzyl ((S)-1-(((S)-1-(((3S,4S,5S)-1-((S)-2-((1R,2R)-3-(((1R,2S)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (23-11) (30 mg, 0.017 mmol) in MeOH (2 mL) was added D-glucose (9.25 mg, 0.051 mmol), followed by NaCNBH3 (2.31 mg, 0.068 mmol). The resulting mixture was stirred and heated at 60° C. for another 0.5 h to complete. Then the completed reaction solution was diluted with H2O (2 mL) and purified by pre-HPLC to give 4-((2S,5S,8S,35S,36R,37R,38R)-8-(6-(2,5-dioxo-2,5-dihydro-1H-pyrrol-1-yl)hexanamido)-35,36,37,38,39-pentahydroxy-5-isopropyl-4,7,14-trioxo-33-((2S,3R,4R,5R)-2,3,4,5,6-pentahydroxyhexyl)-2-(3-ureidopropyl)-15,18,21,24,27,30-hexaoxa-3,6,13,33-tetraazanonatriacontanamido)benzyl ((S)-1-(((S)-1-(((3S,4S,5S)-1-((S)-2-((1R,2R)-3-(((1R,2S)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (23-12) (10 mg, 0.0048 mmol, 28.23%) as a white solid. ESI m/z=695.0 (M/3+H)+, retention time 7.325 min (HPLC). 1H NMR (400 MHz, DMSO-d6) δ 10.03 (s, 1H), 8.40-8.30 (m, 0.5H), 8.19-8.08 (m, 2.5H), 7.98-7.90 (m, 1.5H), 7.66-7.56 (m, 4H), 7.35-7.24 (m, 7H), 7.19-7.16 (m, 1.5H), 5.99 (t, J=6.0 Hz, 1H), 5.44-5.34 (m, 5H), 5.10-4.92 (m, 2H), 4.93-4.60 (m, 2H), 4.60-4.26 (m, 10H), 4.17-4.06 (m, 3H), 4.03-3.93 (m, 6H), 3.78-3.72 (m, 2H), 3.67-3.63 (m, 2H), 3.63-3.42 (m, 25H), 3.35-2.83 (m, 17H), 2.60 (s, 4H), 2.43-2.37 (m, 1H), 2.30-2.22 (m, 1H), 2.15-2.03 (m, 4H), 2.03-1.92 (m, 2H), 1.84-1.70 (m, 7H), 1.53-1.23 (m, 13H), 1.23-1.05 (m, 5H), 1.01-0.96 (m, 7H), 0.96-0.74 (m, 29H) ppm.

### Example 24: Preparation of an Exemplary Linker Drug

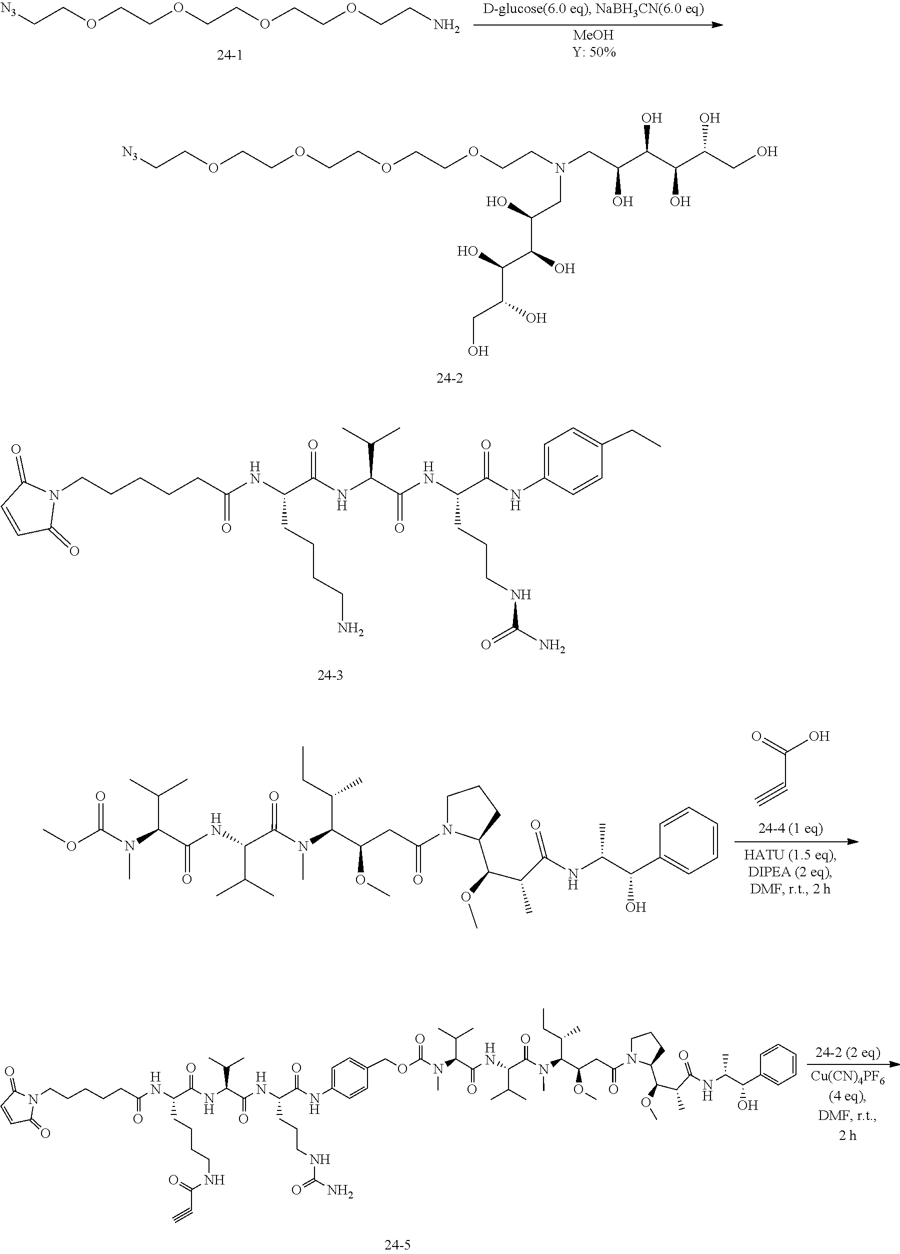

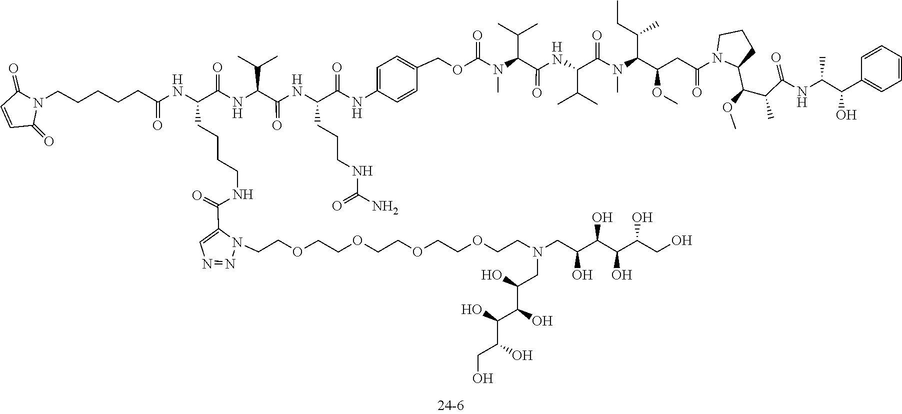 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCCNC(=O)c2cnnn2CCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN2C(=O)C=CC2=O)C(C)C)cc1)C(C)C]

An Exemplary Linker Drug was prepared as follows:

### Step 1

To the solution of 14-azido-3,6,9,12-tetraoxatetradecan-1-amine (24-1) (200 mg, 0.762 mmol) in MeOH (10 mL) was added D-Glucose (824.19 mg, 4.575 mmol) and NaBH3CN (287.48 mg, 4.575 mmol). The mixture was stirred at 70° C. for overnight. Then the completed solution was purified by reverse phase flash chromatography (C8 column, eluting with 0-5 acetonitrile in water with TFA) to afford the product (17S,18R,19R,20R)-1-azido-15-((2S,3R,4R,5R)-2,3,4,5,6-pentahydroxyhexyl)-3,6,9,12-tetraoxa-15-azahenicosane-17,18,19,20,21-pentaol (24-2) (225 mg, 0.381 mmol, 49.960%) as transparent oil. ESI m/z: 591.3 (M+H)+.

### Step 2

To the solution of prop-2-ynoic acid (24-4) (0.006 mL, 0.104 mmol) in DMF (5 mL) was added HATU (59.21 mg, 0.156 mmol) and DIPEA (26.83 mg, 0.208 mmol), then stirred for 5 min, then {4-[(2S)-2-[(2S)-2-[(2S)-6-amino-2-[6-(2,5-dioxo-2,5-dihydro-1H-pyrrol-1-yl)hexanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (24-3) (MC-KVC-pab-MMAE, 150 mg, 0.104 mmol). The mixture was stirred at room temperature for 2 h. The resulting solution was purified by reverse phase flash chromatography (C8 column, eluting with 0-45% acetonitrile in water with 0.01% TFA) to afford the product {4-[(2S)-5-(carbamoylamino)-2-[(2S)-2-[(2S)-2-[6-(2,5-dioxo-2,5-dihydro-1H-pyrrol-1-yl)hexanamido]-6-(prop-2-ynamido)hexanamido]-3-methylbutanamido]pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (24-5) (61 mg, 0.041 mmol, 39.25%) as a white solid. ESI m/z: 748.9 (M/2+H)+.

### Step 3

To the solution of {4-[(2S)-5-(carbamoylamino)-2-[(2S)-2-[(2S)-2-[6-(2,5-dioxo-2,5-dihydro-1H-pyrrol-1-yl)hexanamido]-6-(prop-2-ynamido)hexanamido]-3-methylbutanamido]pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (24-5) (61 mg, 0.041 mmol) in DMF (4 mL) was added (17S,18R,19R,20R)-1-azido-15-((2S,3R,4R,5R)-2,3,4,5,6-pentahydroxyhexyl)-3,6,9,12-tetraoxa-15-azahenicosane-17,18,19,20,21-pentaol (24-2) (48.14 mg, 0.082 mmol) and Cu(CH₃CN)₄PF₆ (61.13 mg, 0.164 mmol). The mixture was stirred at room temperature for 2 h. Then the resulting solution was purified directly by reverse phase flash chromatography (C8 column, eluting with 0-40% acetonitrile in water with 0.01% TFA) to afford the product {4-[(2S)-5-(carbamoylamino)-2-[(2S)-2-[(2S)-2-[6-(2,5-dioxo-2,5-dihydro-1H-pyrrol-1-yl)hexanamido]-6-({1-[(17S,18R,19R,20R)-17,18,19,20,21-pentahydroxy-15-[(2S,3R,4R,5R)-2,3,4,5,6-pentahydroxyhexyl]-3,6,9,12-tetraoxa-15-azahenicosan-1-yl]-1H-1,2,3-triazol-5-yl}formamido)hexanamido]-3-methylbutanamido]pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (24-6) (35 mg, 0.017 mmol, 41.14%) as a white solid. ESI m/z: 696.5 (M/3+H)+, retention time 7.297 min (HPLC). 1H NMR (400 MHz, DMSO-d6) δ 10.03 (s, 1H), 8.49-8.46 (m, 1H), 8.35-8.25 (m, 0.5H), 8.13-8.10 (m, 2H), 8.10-8.07 (m, 1H), 7.98-7.89 (m, 1.5H), 7.69-7.64 (m, 1H), 7.56 (d, J=7.6 Hz, 2H), 7.31-7.23 (m, 6H), 7.23-7.15 (m, 1H), 6.98 (s, 2H), 6.01 (t, J=6.0 Hz, 1H), 5.43 (brs, 4H), 5.10-4.93 (m, 3H), 4.93-4.57 (m, 2H), 4.57-4.25 (m, 10H), 4.25-4.16 (m, 6H), 4.03-3.98 (m, 5H), 3.93-3.89 (m, 2H), 3.84-3.81 (m, 4H), 3.76-3.73 (m, 4H), 3.68-3.53 (m, 14H), 3.35-3.20 (m, 8H), 0.20-2.82 (m, 12H), 2.40-2.00 (m, 7H), 2.00-1.85 (m, 2H), 1.85-1.20 (m, 26H), 1.05-0.95 (m, 7H), 0.95-0.70 (m, 28H) ppm.

### Example 25: Preparation of an Exemplary Linker Drug

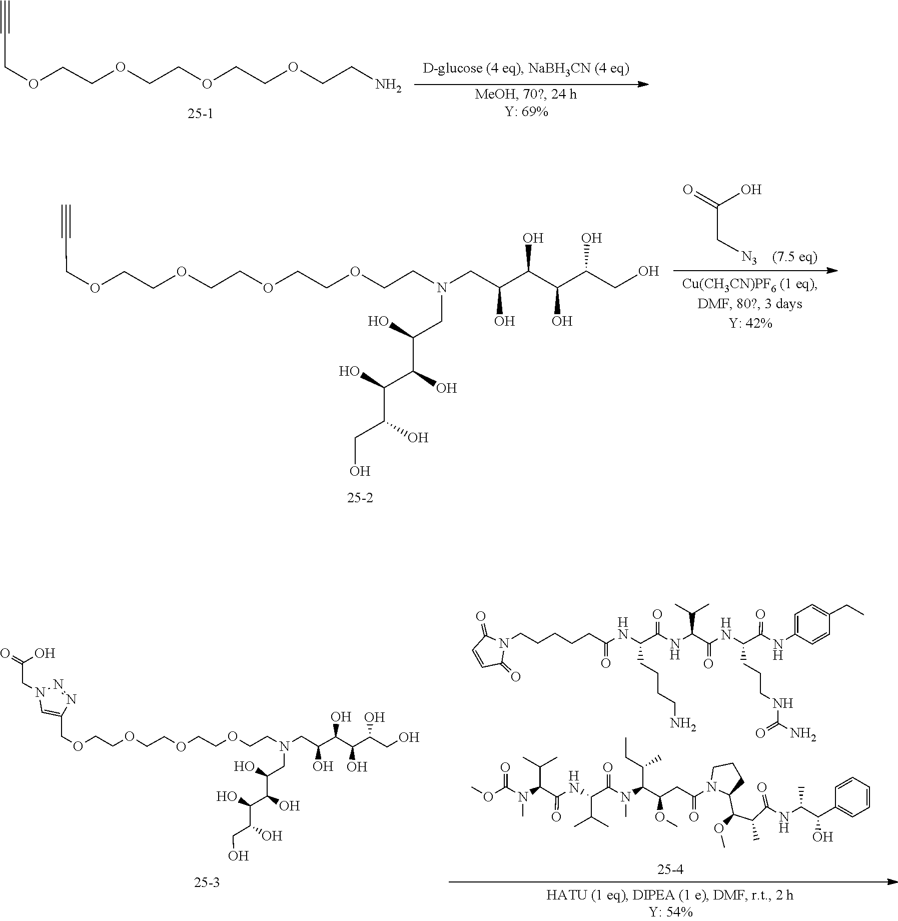

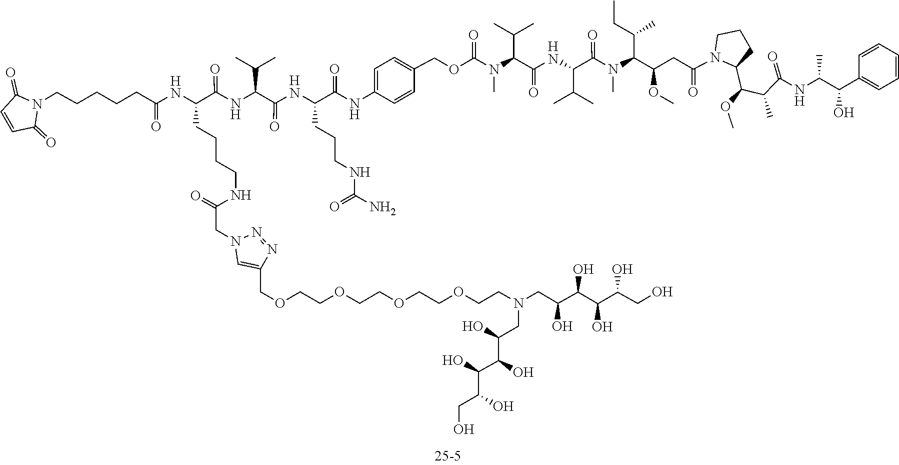 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCCNC(=O)Cn2cc(COCCOCCOCCOCCN(C[C@H](O)[C@@H](O)[C@H](O)[C@H](O)CO)C[C@H](O)[C@@H](O)[C@H](O)[C@H](O)CO)nn2)NC(=O)CCCCCN2C(=O)C=CC2=O)C(C)C)cc1)C(C)C]

An Exemplary Linker Drug was prepared as follows:

### Step 1

To the solution of 3,6,9,12-tetraoxapentadec-14-yn-1-amine (25-1) (100 mg, 0.432 mmol) in MeOH (5 mL) was added D-Glucose (311.54 mg, 1.729 mmol) and NaBH3CN (108.68 mg, 1.729 mmol). The resulting mixture was stirred at 75° C. for 24 h to complete. Then the resulting solution was purified by reverse phase flash chromatography (C8 column, eluting with 0-20% acetonitrile in water with TFA) to afford product (18S,19R,20R,21R)-16-[(2S,3R,4R,5R)-2,3,4,5,6-pentahydroxyhexyl]-4,7,10,13-tetraoxa-16-azadocos-1-yne-18,19,20,21,22-pentol (25-2) (168 mg, 0.300 mmol, 69.44%) as a white gel. ESI m/z: 560.3 (M+H)+.

### Step 2

To the solution of (18S,19R,20R,21R)-16-[(2S,3R,4R,5R)-2,3,4,5,6-pentahydroxyhexyl]-4,7,10,13-tetraoxa-16-azadocos-1-yne-18,19,20,21,22-pentol (25-2) (30 mg, 0.054 mmol) in DMF (2 mL) was added 2-azidoacetic acid (41.11 mg, 0.407 mmol) and Cu(CH₃CN)₄PF₆ (20.13 mg, 0.054 mmol) sequentially. The mixture was heated and stirred at 80° C. over the weekend to complete. Then the resulting solution was purified by reverse phase flash chromatography (C8 column, eluting with 0-10% acetonitrile in water with 0.01% TFA) to afford the product 2-{4-[(16S,17R,18R,19R)-16,17,18,19,20-pentahydroxy-14-[(2S,3R,4R,5R)-2,3,4,5,6-pentahydroxyhexyl]-2,5,8,11-tetraoxa-14-azaicosan-1-yl]-1H-1,2,3-triazol-1-yl}acetic acid (25-3) (15 mg, 0.023 mmol, 42.35%) as transparent oil. ESI m/z: 661.0 (M+H)+.

### Step 3

To the solution of 2-{4-[(16S,17R,18R,19R)-16,17,18,19,20-pentahydroxy-14-[(2S,3R,4R,5R)-2,3,4,5,6-pentahydroxyhexyl]-2,5,8,11-tetraoxa-14-azaicosan-1-yl]-1H-1,2,3-triazol-1-yl}acetic acid (25-3) (5 mg, 0.008 mmol) in DMF (2 mL) was added {4-[(2S)-2-[(2S)-2-[(2S)-6-amino-2-[6-(2,5-dioxo-2,5-dihydro-1H-pyrrol-1-yl)hexanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (25-4) (10.93 mg, 0.008 mmol), followed by HATU (2.88 mg, 0.008 mmol) and DIPEA (0.98 mg, 0.008 mmol). After addition, the mixture was stirred at room temperature for 2 h and completed. Then the resulting solution was purified directly by reverse phase flash chromatography (C8 column, eluting with 0-50% acetonitrile in water with 0.01% TFA) to afford the product {4-[(2S)-5-(carbamoylamino)-2-[(2S)-2-[(2S)-2-[6-(2,5-dioxo-2,5-dihydro-1H-pyrrol-1-yl)hexanamido]-6-(2-{4-[(16S,17R,18R,19R)-16,17,18,19,20-pentahydroxy-14-[(2S,3R,4R,5R)-2,3,4,5,6-pentahydroxyhexyl]-2,5,8,11-tetraoxa-14-azaicosan-1-yl]-1H-1,2,3-triazol-1-yl}acetamido)hexanamido]-3-methylbutanamido]pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (25-5) (8.6 mg, 0.004 mmol, 54.43%) as a pale yellow solid. ESI m/z: 696.8 (M/3+H)+, retention time 7.288 min (HPLC) 1H NMR (400 MHz, DMSO-d6) δ 10.02 (s, 1H), 8.33-8.31 (m, 1H), 8.15-8.07 (m, 2.5H), 8.01 (s, 1H), 7.98-7.89 (m, 1.5H), 7.68-7.63 (m, 1H), 7.57-7.55 (m, 2H), 7.31-7.23 (m, 6H), 7.19-7.14 (m, 1H), 6.99 (s, 2H), 5.99 (t, J=4.8 Hz, 1H), 5.43 (brs, 4H), 5.08-4.97 (m, 3H), 4.82-4.72 (m, 2H), 4.60-4.36 (m, 11H), 4.28-4.17 (m, 3H), 4.03-3.93 (m, 4H), 3.80-3.72 (m, 2H), 3.72-3.60 (m, 2H), 3.55-3.37 (m, 22H), 3.24-3.17 (m, 8H), 3.17-2.82 (m, 12H), 2.33-2.81 (m, 1H), 2.14-1.94 (m, 7H), 1.83-1.15 (m, 28H), 1.05-0.96 (m, 7H), 0.86-0.74 (m, 28H) ppm.

### Example 26: Preparation of an Exemplary Linker Drug

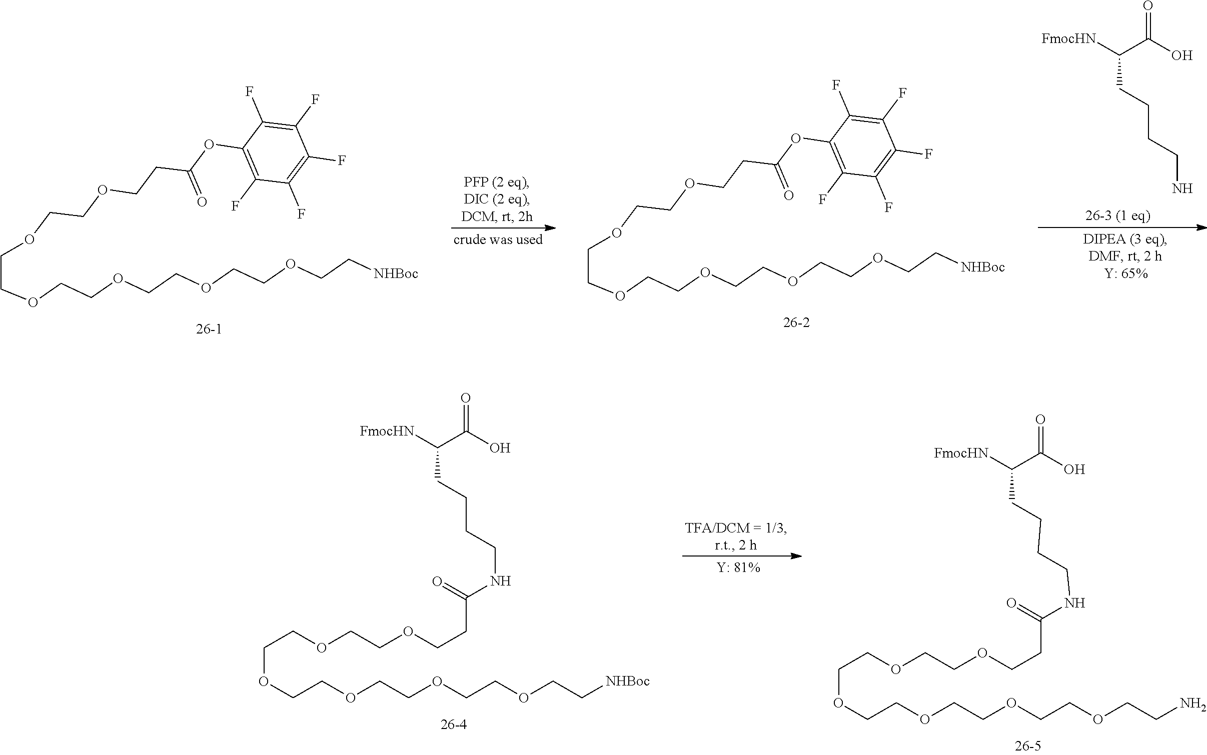

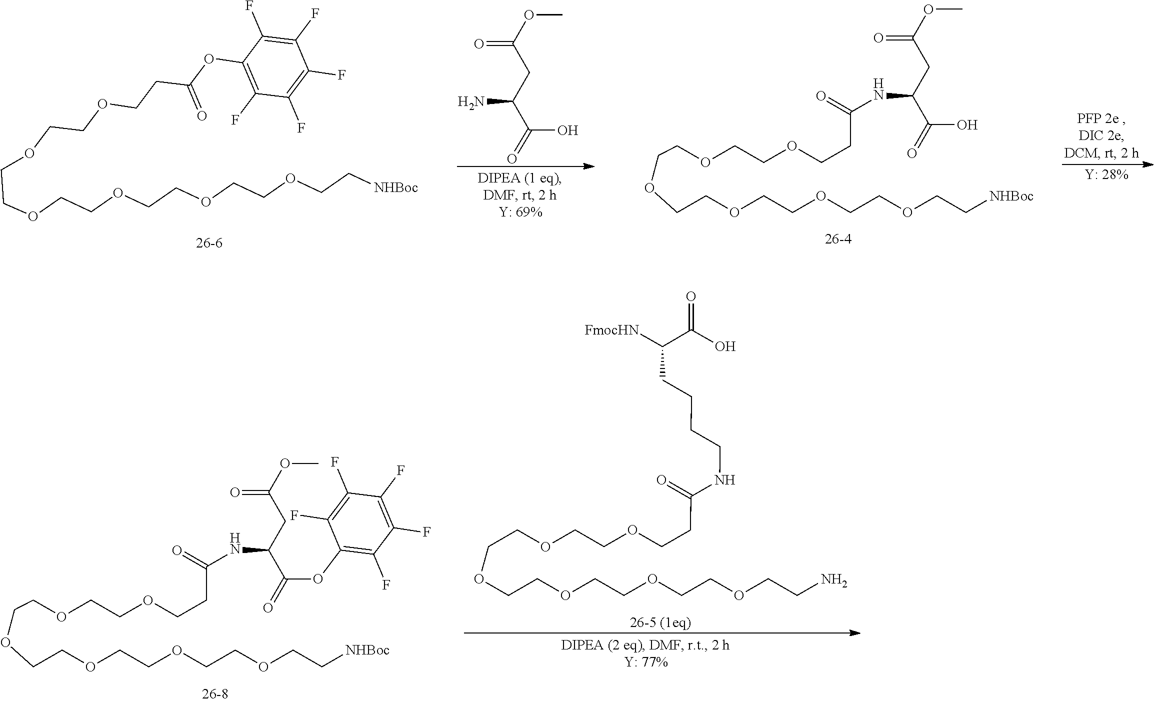

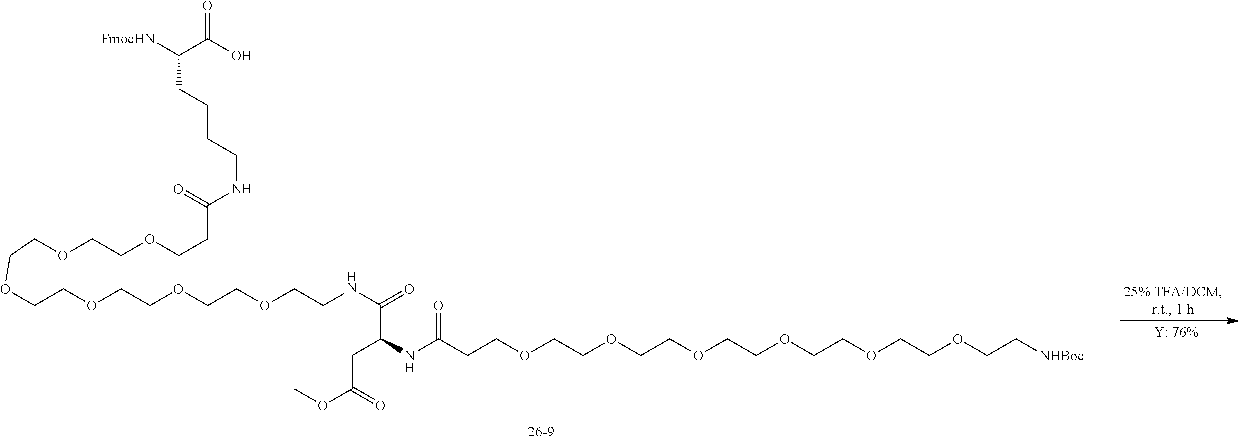

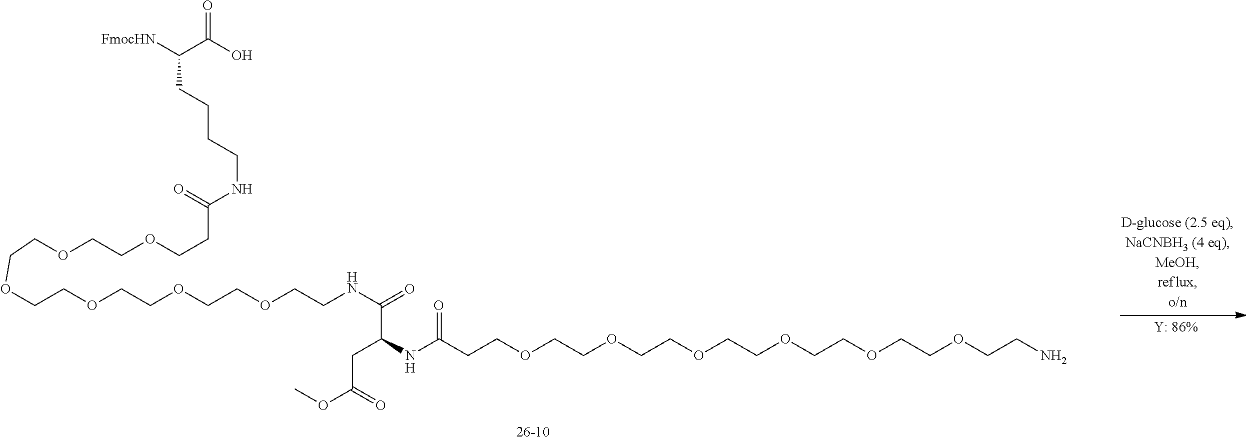

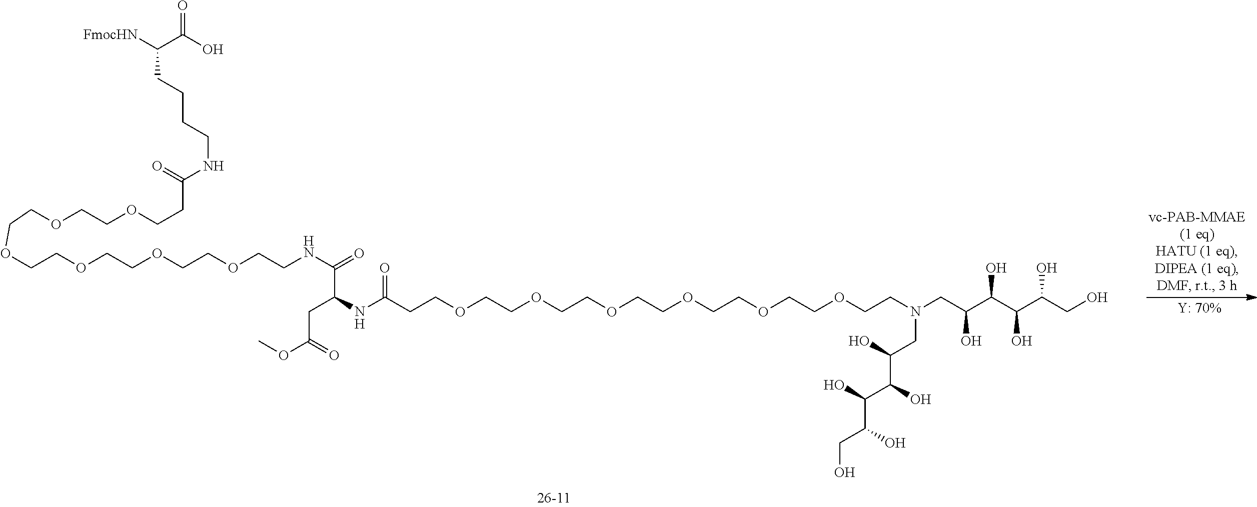

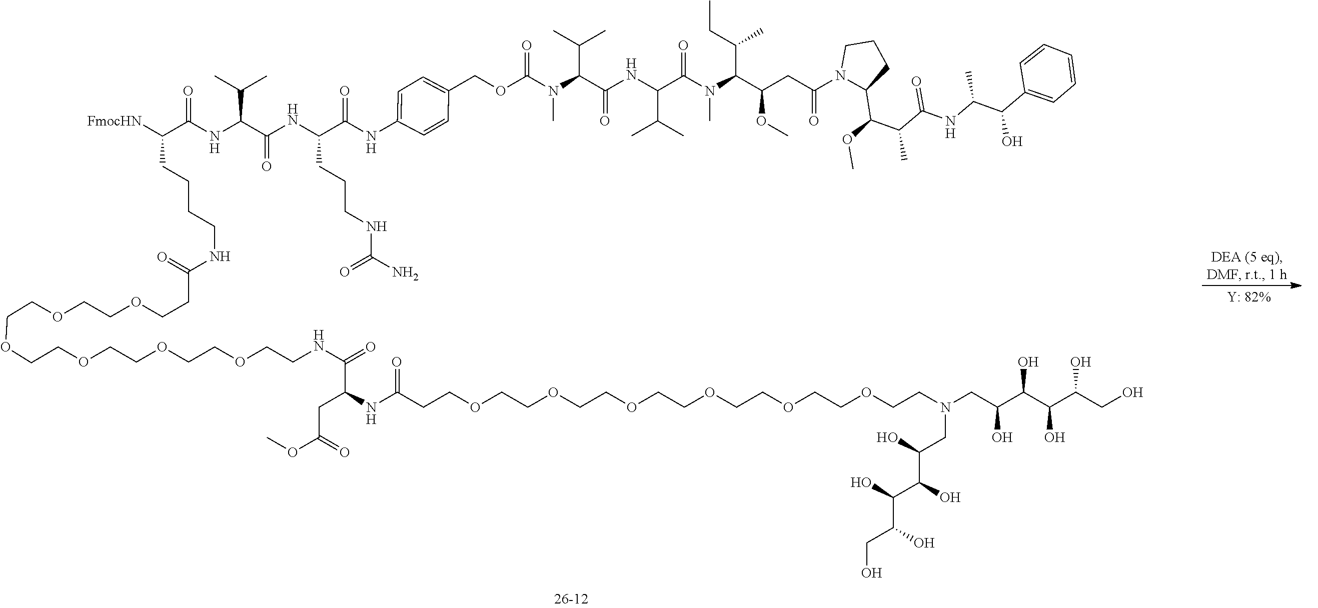

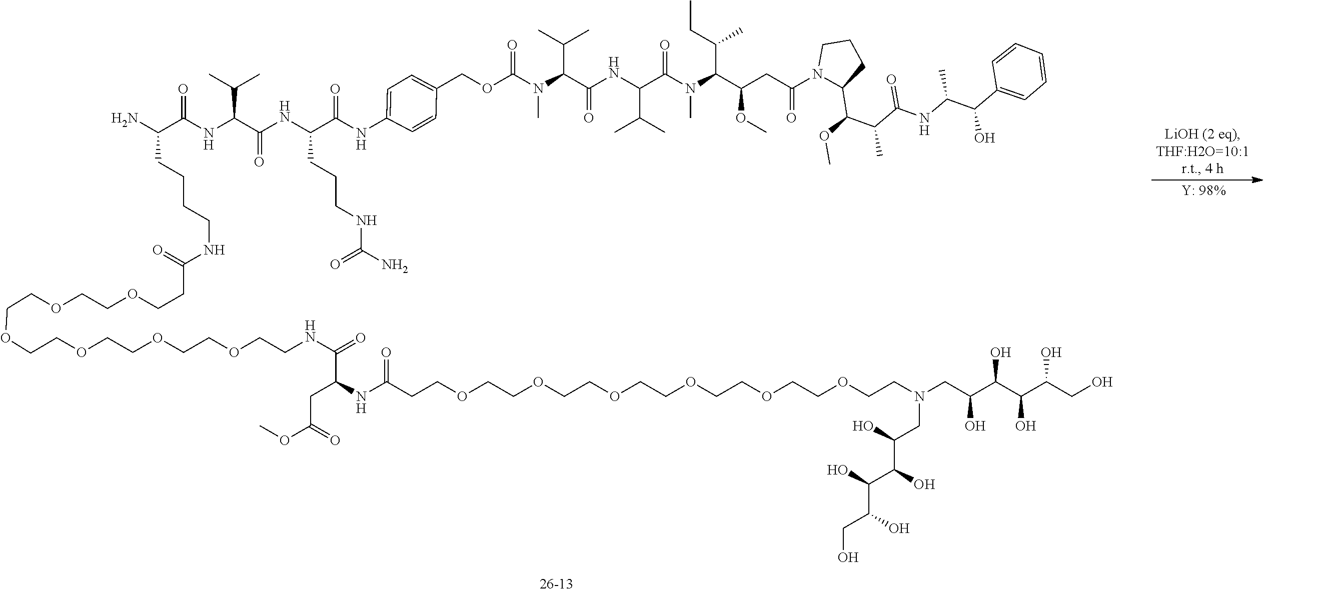 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)C(NC(=O)[C@H](C(C)C)N(C)C(=O)OCc1ccc(NC(=O)[C@H](CCCNC(N)=O)NC(=O)[C@@H](NC(=O)[C@@H](N)CCCCNC(=O)CCOCCOCCOCCOCCOCCOCCNC(=O)[C@H](CC(=O)OC)NC(=O)CCOCCOCCOCCOCCOCCOCCN(C[C@H](O)[C@@H](O)[C@H](O)[C@H](O)CO)C[C@H](O)[C@@H](O)[C@H](O)[C@H](O)CO)C(C)C)cc1)C(C)C]

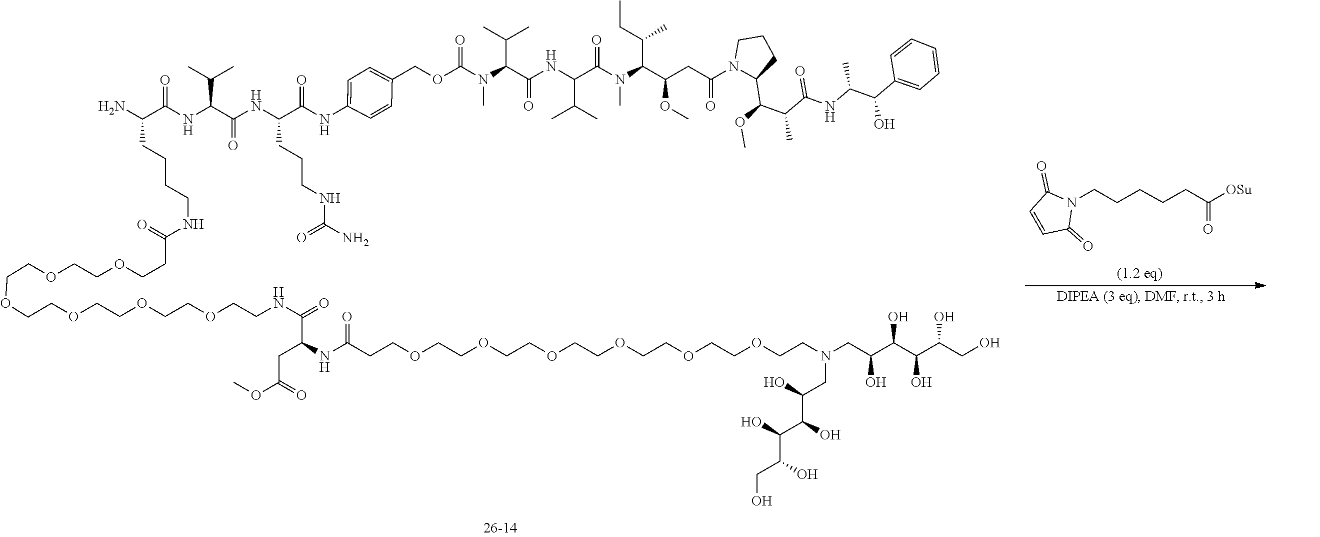

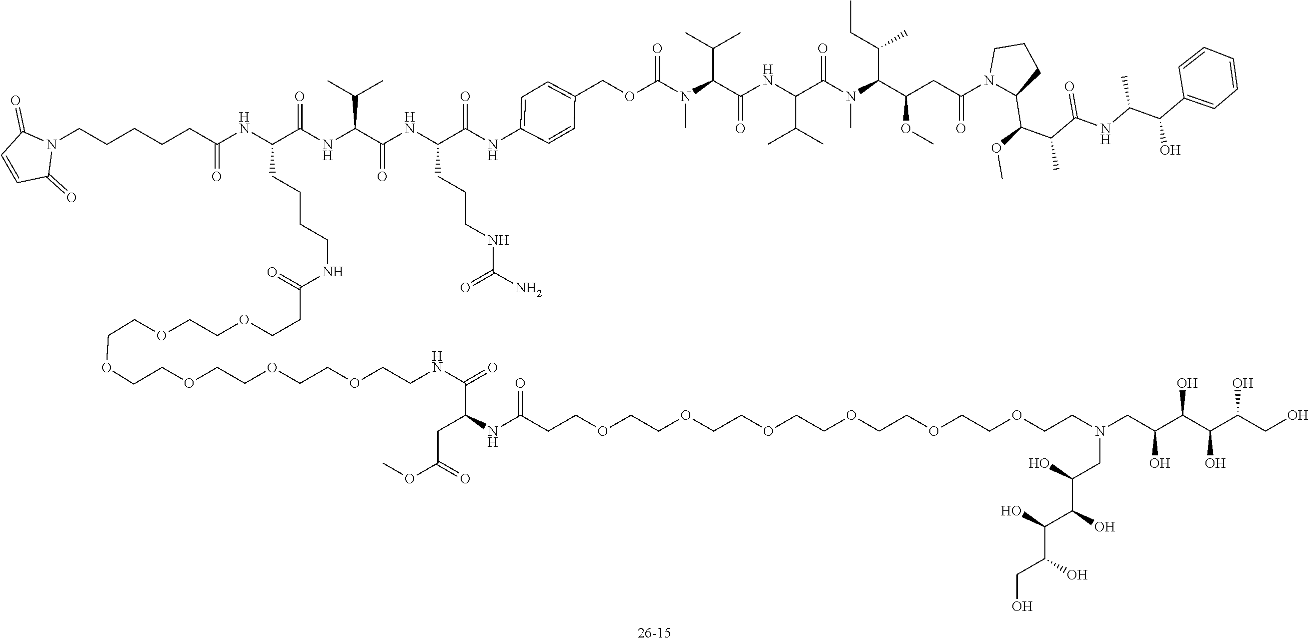 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)C(NC(=O)[C@H](C(C)C)N(C)C(=O)OCc1ccc(NC(=O)[C@H](CCCNC(N)=O)NC(=O)[C@@H](NC(=O)[C@H](CCCCNC(=O)CCOCCOCCOCCOCCOCCOCCNC(=O)[C@H](CC(=O)OC)NC(=O)CCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN2C(=O)C=CC2=O)C(C)C)cc1)C(C)C]

A Drug-Linker attached to exatecan for further attachment to a Polar Group is prepared as follows:

### Step 1

To a solution of 1-{[(tert-butoxy)carbonyl]amino}-3,6,9,12,15,18-hexaoxahenicosan-21-oic acid (26-1) (500 mg, 1.102 mmol) in DCM (20 mL) was added pentafluorophenol (405.84 mg, 2.205 mmol) and N,N′-Diisopropylcarbodiimide (0.344 mL, 2.205 mmol). The reaction mixture was stirred at 25° C. for 2 h under Ar until all materials were consumed. The solution was then washed with water and extracted with more DCM (20 mL*2). The organic layer was combined and dried over sodium sulfate, filtered and concentrated to give crude pentafluorophenyl 1-{[(tert-butoxy)carbonyl]amino}-3,6,9,12,15,18-hexaoxahenicosan-21-oate (26-2) (590 mg, 0.952 mmol, 86.38%) as a white solid. ESI m/z=642.3 (M+Na)+.

### Step 2

To a solution of (2S)-6-amino-2-({[(9H-fluoren-9-yl)methoxy]carbonyl}amino)hexanoic acid (26-3) (344.89 mg, 0.936 mmol) in DMF (3 mL) was added DIPEA (0.464 mL, 2.808 mmol) and pentafluorophenyl 1-{[(tert-butoxy)carbonyl]amino}-3,6,9,12,15,18-hexaoxahenicosan-21-oate (26-2) (580 mg, 0.936 mmol). The reaction mixture was stirred at 25° C. for 2 h under Ar. Then the reaction mixture was purified by reverse phase column chromatography (C18 column chromatography, 0-70% acetonitrile in water with 0.01% TFA) to give product (2S)-6-(1-{[(tert-butoxy)carbonyl]amino}-3,6,9,12,15,18-hexaoxahenicosan-21-amido)-2-({[(9H-fluoren-9-yl)methoxy]carbonyl}amino)hexanoic acid (26-4) (490 mg, 0.609 mmol, 65.11%) as a white solid. ESI m/z=804.1 (M+H)+, 826.1 (M+Na)+.

### Step 3

A solution of (2S)-6-(1-{[(tert-butoxy)carbonyl]amino}-3,6,9,12,15,18-hexaoxahenicosan-21-amido)-2-({[(9H-fluoren-9-yl)methoxy]carbonyl}amino)hexanoic acid (26-4) (490 mg, 0.609 mmol) in TFA (2 mL) and THF (6 mL) was stirred at 25° C. for 2 h under Ar. Then the completed solution was concentrated under vacuo to remove solvent. The residue was further purified by reverse phase column chromatography (C18 column chromatography, 0-60% acetonitrile in water with 0.010% TFA) to give (2S)-6-(1-amino-3,6,9,12,15,18-hexaoxahenicosan-21-amido)-2-({[(9H-fluoren-9-yl)methoxy]carbonyl}amino)hexanoic acid (26-5) (350 mg, 0.497 mmol, 81.59%) as a white solid. ESI m/z=704.3 (M+H)+,

### Step 4

To a solution of (2S)-2-amino-4-methoxy-4-oxobutanoic acid (127.99 mg, 0.870 mmol) in DMF (3 mL) was added DIPEA (0.131 mL, 0.791 mmol) and pentafluorophenyl 1-{[(tert-butoxy)carbonyl]amino}-3,6,9,12,15,18-hexaoxahenicosan-21-oate (26-6) (490 mg, 0.791 mmol). The reaction mixture was stirred at 25° C. for 2 h under Ar. The completed reaction mixture was purified directly by reverse phase column chromatography (C18 column chromatography, 0-70% acetonitrile in water with 0.01% TFA) to give (2S)-2-(1-{[(tert-butoxy)carbonyl]amino}-3,6,9,12,15,18-hexaoxahenicosan-21-amido)-4-methoxy-4-oxobutanoic acid (26-7) (320 mg, 0.549 mmol, 69.45%) as a white solid, impure, used as such in next step. ESI m/z=583.3 (M+H)+, 605.2 (M+Na)+.

### Step 5

To a solution of(2S)-2-(1-{[(tert-butoxy)carbonyl]amino}-3,6,9,12,15,18-hexaoxahenicosan-21-amido)-4-methoxy-4-oxobutanoic acid (26-7) (300 mg, 0.515 mmol) in DCM (5 mL) was added pentafluorophenol (189.54 mg, 1.030 mmol) and N,N′-Diisopropylcarbodiimide (0.160 mL, 1.030 mmol). The reaction mixture was stirred at 25° C. for 2 h under Ar. After completion, the solution was concentrated under vacuo. The residue was purified by flash chromatography (silica gel, PE/EA=10:1) to give 1-methyl pentafluorophenyl (3S)-3-(1-{[(tert-butoxy)carbonyl]amino}-3,6,9,12,15,18-hexaoxahenicosan-21-amido)butanedioate (26-8) (110 mg, 0.147 mmol, 28.53%) as a white solid. ESI m/z=771.1 (M+Na)+, 649.3 (M−100+H)+.

### Step 6

To a solution of (2S)-6-(1-amino-3,6,9,12,15,18-hexaoxahenicosan-21-amido)-2-({[(9H-fluoren-9-yl)methoxy]carbonyl}amino)hexanoic acid (26-5) (100 mg, 0.142 mmol) in DMF (3 mL) was added DIPEA (0.070 mL, 0.426 mmol) and 1-methyl pentafluorophenyl (3S)-3-(1-{[(tert-butoxy)carbonyl]amino}-3,6,9,12,15,18-hexaoxahenicosan-21-amido)butanedioate (26-8) (106.37 mg, 0.142 mmol). Then the reaction mixture was stirred at 25° C. for 2 h to complete. The completed solution was concentrated to dryness and then purified by flash chromatography (silica gel, PE/EA=10:1) to give (2S)-6-{1-[(2S)-2-(1-{[(tert-butoxy)carbonyl]amino}-3,6,9,12,15,18-hexaoxahenicosan-21-amido)-4-methoxy-4-oxobutanamido]-3,6,9,12,15,18-hexaoxahenicosan-21-amido}-2-({[(9H-fluoren-9-yl)methoxy]carbonyl}amino)hexanoic acid (26-9) (140 mg, 0.110 mmol, 77.68%) as a white solid. LCMS: m/z=1269.4 (M+H)+, 584.8 ((M−100)/2+H)+.

### Step 7

A solution of(2S)-6-{1-[(2S)-2-(1-{[(tert-butoxy)carbonyl]amino}-3,6,9,12,15,18-hexaoxahenicosan-21-amido)-4-methoxy-4-oxobutanamido]-3,6,9,12,15,18-hexaoxahenicosan-21-amido}-2-({[(9H-fluoren-9-yl)methoxy]carbonyl}amino)hexanoic acid (26-9) (140 mg, 0.110 mmol) in TFA (1 mL) and DCM (3 mL) was stirred at 25° C. for 1 h until LCMS indicated complete deprotection. Then the solution was concentrated to dryness and then purified by reverse phase column chromatography (C18 column, 0-50% acetonitrile in water with 0.01% TFA) to give title (2S)-6-{1-[(2S)-2-(1-amino-3,6,9,12,15,18-hexaoxahenicosan-21-amido)-4-methoxy-4-oxobutanamido]-3,6,9,12,15,18-hexaoxa henicosan-21-amido}-2-({[(9H-fluoren-9-yl)methoxy]carbonyl}amino)hexanoic acid (26-10) (100 mg, 0.086 mmol, 77.55%) as a white solid. LCMS: m/z=1169. 4 (M+H)+, 584.8 (M/2+H)+.

### Step 8

To a solution of (23S,52S)-52-((((9H-fluoren-9-yl)methoxy)carbonyl)amino)-1-amino-23-(2-methoxy-2-oxoethyl)-21,24,46-trioxo-3,6,9,12,15,18,28,31,34,37,40,43-dodecaoxa-22,25,47-triazatripentacontan-53-oic acid (90 mg, 0.077 mmol) in MeOH (5 mL) was added D-glucose (34.7 mg, 0.193 mmol) followed by NaCNBH3 (10.41 mg, 0.308 mmol). The resulting mixture was heated at 60° C. for overnight to achieve complete conversion. Then the reaction mixture was diluted with water (2 mL) and then purified by pre-HPLC to give product (2R,3R,4R,5S,30S,59S)-59-((((9H-fluoren-9-yl)methoxy)carbonyl)amino)-1,2,3,4,5-pentahydroxy-30-(2-methoxy-2-oxoethyl)-28,31,53-trioxo-7-((2S,3R,4R,5R)-2,3,4,5,6-pentahydroxyhexyl)-10,13,16,19,22,25,35,38,41,44,47,50-dodecaoxa-7,29,32,54-tetraazahexacontan-60-oic acid (26-11) (100 mg, 0.0668 mmol, 86.77%) as a white solid. LCMS: m/z=749.1 (M/2+H)+.

### Step 9

To a solution of (2S)-2-({[(9H-fluoren-9-yl)methoxy]carbonyl}amino)-6-{1-[(2S)-4-methoxy-4-oxo-2-[(24S,25R,26R,27R)-24,25,26,27,28-pentahydroxy-22-[(2S,3R,4R,5R)-2,3,4,5,6-pentahydroxyhexyl]-4,7,10,13,16,19-hexaoxa-22-azaoctacosanamido]butanamido]-3,6,9,12,15,18-hexaoxahenicosan-21-amido}hexanoic acid (26-11) (90 mg, 0.060 mmol) in DMF (5 ml) was added {4-[(2S)-2-[(2S)-2-amino-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (vo-PAB-MMAE) (67.56 mg, 0.060 mmol), followed by HATU (22.86 mg, 0.060 mmol) and DIPEA (0.010 mL, 0.060 mmol) sequentially. The resulting solution was stirred at 25° C. for 3 h. Then the completed reaction mixture was purified directly by reverse phase column chromatography (C8 column, 0-100% acetonitrile in water with 0.01% TFA) to give methyl (3S)-3-[(20-{[(5S)-5-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}-5-({[(9H-fluoren-9-yl)methoxy]carbonyl}amino)pentyl]carbamoyl}-3,6,9,12,15,18-hexaoxaicosan-1-yl)carbamoyl]-3-[(24S,25R,26R,27R)-24,25,26,27,28-pentahydroxy-22-[(2S,3R,4R,5R)-2,3,4,5,6-pentahydroxyhexyl]-4,7,10,13,16,19-hexaoxa-22-azaoctacosanamido]propanoate (26-12) (110 mg, 0.042 mmol, 70.30%) as a white solid. LCMS: m/z=868.3 (M/3+H)+.

### Step 10

To a solution of methyl (3S)-3-[(20-{[(5S)-5-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}-5-({[(9H-fluoren-9-yl)methoxy]carbonyl}amino)pentyl]carbamoyl}-3,6,9,12,15,18-hexaoxaicosan-1-yl)carbamoyl]-3-[(24S,25R,26R,27R)-24,25,26,27,28-pentahydroxy-22-[(2S,3R,4R,5R)-2,3,4,5,6-pentahydroxyhexyl]-4,7,10,13,16,19-hexaoxa-22-azaoctacosanamido]propanoate (26-12) (120 mg, 0.046 mmol) in DMF (5 mL) was added diethylamine (0.024 mL, 0.231 mmol). Then the reaction mixture was stirred at 25° C. for 1 h to achieve completion. Then the mixture was purified directly by reverse phase column chromatography (C18 column chromatography, 0-100% acetonitrile in water with 0.01% TFA) to give methyl (3S)-3-[(20-{[(5S)-5-amino-5-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}pentyl]carbamoyl}-3,6,9,12,15,18-hexaoxaicosan-1-yl)carbamoyl]-3-[(24S,25R,26R,27R)-24,25,26,27,28-pentahydroxy-22-[(2S,3R,4R,5R)-2,3,4,5,6-pentahydroxyhexyl]-4,7,10,13,16,19-hexaoxa-22-azaoctacosanamido]propanoate (26-13) (90 mg, 0.038 mmol, 82.00%) as a white solid. ESI m/z=783.5 ((M−32)/3+H)+, mass of lactone), 794.1 (M/3+H)+.

### Step 11

To a solution of methyl (3S)-3-[(20-{[(5S)-5-amino-5-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}pentyl]carbamoyl}-3,6,9,12,15,18-hexaoxaicosan-1-yl)carbamoyl]-3-[(24S,25R,26R,27R)-24,25,26,27,28-pentahydroxy-22-[(2S,3R,4R,5R)-2,3,4,5,6-pentahydroxyhexyl]-4,7,10,13,16,19-hexaoxa-22-azaoctacosanamido]propanoate (26-13) (80 mg, 0.034 mmol) in THF (2 mL) was added a solution of LiOH (0.002 mL, 0.067 mmol) in water (0.2 mL). Then the reaction mixture was stirred at 25° C. for 4 h. After complete hydrolysis, the mixture was concentrated to remove THF, and the residue was purified by reverse phase column chromatography (C8 column chromatography, 0-100% acetonitrile in water with 0.01% TFA) to give (3S)-3-[(20-{[(5S)-5-amino-5-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1- hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}pentyl]carbamoyl}-3,6,9,12,15,18-hexaoxaicosan-1-yl)carbamoyl]-3-[(24S,25R,26R,27R)-24,25,26,27,28-pentahydroxy-22-[(2S,3R,4R,5R)-2,3,4,5,6-pentahydroxyhexyl]-4,7,10,13,16,19-hexaoxa-22-azaoctacosanamido]propanoic acid (26-14) (78 mg, 0.033 mmol, 98.08%) as a white solid. ESI m/z=592.5 (M/4+H)+, 789.5 (M/3+H)+.

### Step 12

To a solution of (3S)-3-[(20-{[(5S)-5-amino-5-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}pentyl]carbamoyl}-3,6,9,12,15,18-hexaoxaicosan-1-yl)carbamoyl]-3-[(24S,25R,26R,27R)-24,25,26,27,28-pentahydroxy-22-[(2S,3R,4R,5R)-2,3,4,5,6-pentahydroxyhexyl]-4,7,10,13,16,19-hexaoxa-22-azaoctacosanamido]propanoic acid (26-14) (75 mg, 0.032 mmol) in DMF (3 mL) was added DIPEA (0.016 mL, 0.095 mmol), followed by 2,5-dioxopyrrolidin-1-yl 6-(2,5-dioxo-2,5-dihydro-1H-pyrrol-1-yl)hexanoate (11.73 mg, 0.038 mmol) at room temperature. The resulting reaction solution was stirred at 25° C. for another 3 h to complete. Then the reaction solution was purified directly by prep-HPLC (C18 column, 10-95% acetonitrile in water with 0.05% TFA) to obtain (3S)-3-[(20-{[(5S)-5-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}-5-[6-(2,5-dioxo-2,5-dihydro-1H-pyrrol-1-yl)hexanamido]pentyl]carbamoyl}-3,6,9,12,15,18-hexaoxaicosan-1-yl)carbamoyl]-3-[(24S,25R,26R,27R)-24,25,26,27,28-pentahydroxy-22-[(2S,3R,4R,5R)-2,3,4,5,6-pentahydroxyhexyl]-4,7,10,13,16,19-hexaoxa-22-azaoctacosanamido]propanoic acid (26-15) (51 mg, 0.020 mmol, 62.87%) as a white solid. ESI m/z=640.7 (M/4+H)+, 853.9 (M/3+H)+, retention time 7.281 min (HPLC). 1H NMR (400 MHz, DMSO-d6): δ 10.03 (s, 1H), 8.13 (t, J=6.8 Hz, 2H), 8.08 (d, J=8.0 Hz, 1H), 7.98-7.94 (m, 2H), 7.92-7.88 (m, 0.5H), 7.81 (t, J=5.2 Hz, 1H), 7.68-7.64 (m, 1H), 7.60-7.54 (m, 2H), 7.35-7.23 (m, 7H), 7.20-7.14 (m, 0.5H), 6.99 (s, 2H), 6.02-5.95 (m, 1H), 5.50-5.39 (m, 3H), 5.10-4.96 (m, 2H), 4.54-4.46 (m, 3H), 4.42 (d, J=6.4 Hz, 1H), 4.39-4.34 (m, 2H), 4.29-4.16 (m, 4H), 4.03-3.94 (m, 5H), 3.80-3.75 (m, 4H), 3.70-3.64 (m, 4H), 3.61-3.60 (m, 2H), 3.59-3.54 (m, 15H), 3.53-3.46 (m, 50H), 3.25-3.15 (m, 12H), 3.11 (s, 2H), 3.04-2.95 (m, 5H), 2.90-2.81 (m, 3H), 2.39-2.32 (m, 4H), 2.28 (t, J=6.4 Hz, 3H), 2.13-2.06 (m, 4H), 2.00-1.92 (m, 2H), 1.51-1.42 (m, 8H), 1.39-1.31 (m, 4H), 1.21-1.14 (m, 3H), 1.05-0.96 (m, 7H), 0.89-0.70 (m, 31H) ppm.

### Example 27: Preparation of an Exemplary Linker Drug

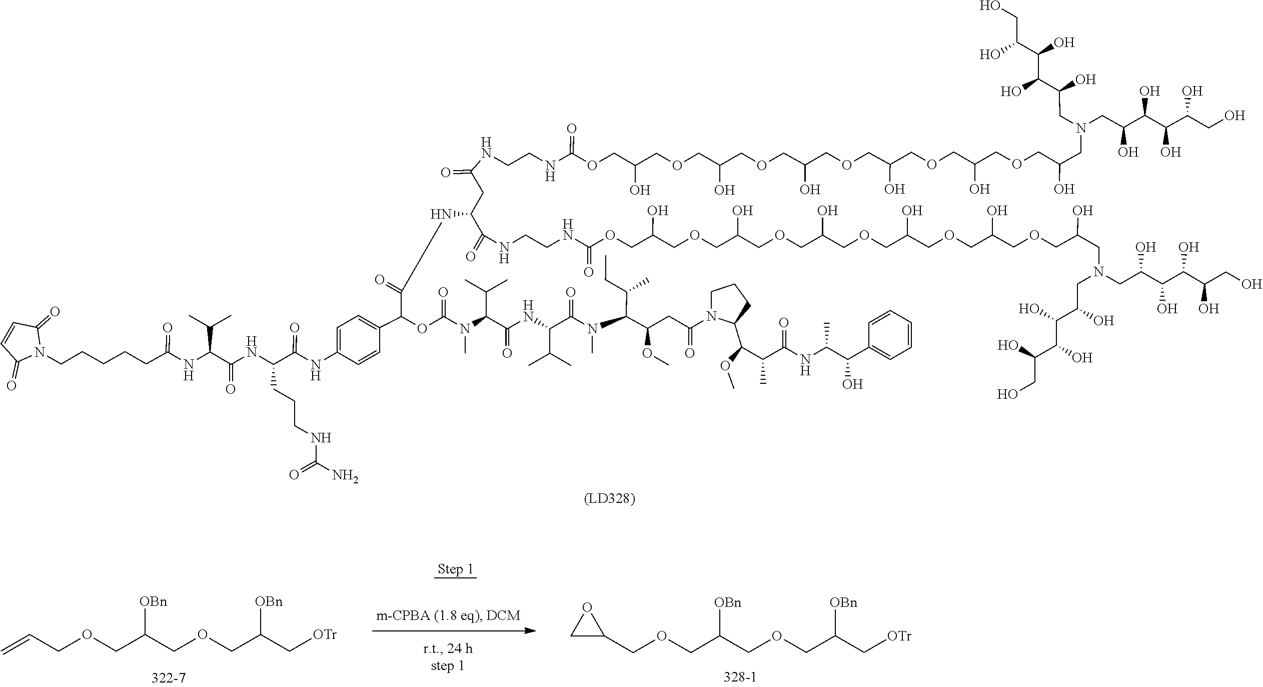

To a solution of 322-7 (10.0 g, 15.915 mmol) in DCM (20 mL) was added a solution of m-CPBA (4.94 g, 28.648 mmol) in DCM (40 mL) dropwise at room temperature. The resulting mixture was stirred at this temperature for 24 hours until 322-7 was consumed, and the reaction was quenched with saturated Na₂S₂O₃ (aq., 20 mL) and NaHCO₃ (aq, 20 mL). The reaction mixture was stirred for 30 mins, and then diluted with DCM (100 mL). The organic phase was washed with a mixture solution (20 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-90% ethyl acetate in petroleum ether) affording 328-1 (9.1 g, 14.12 mmol, 88.7%) as a pale yellow oil. Purity=90%-95%. ¹H NMR (400 MHz, CDCl₃) δ 7.47-7.38 (m, 5H), 7.36-7.03 (m, 20H), 4.70-4.39 (m, 4H), 3.79-3.41 (m, 9H), 3.40-3.32 (m, 1H), 3.28-3.14 (m, 2H), 3.12-3.01 (m, 1H), 2.79-2.66 (m, 1H), 2.60-2.47 (m, 1H).

### Step 2

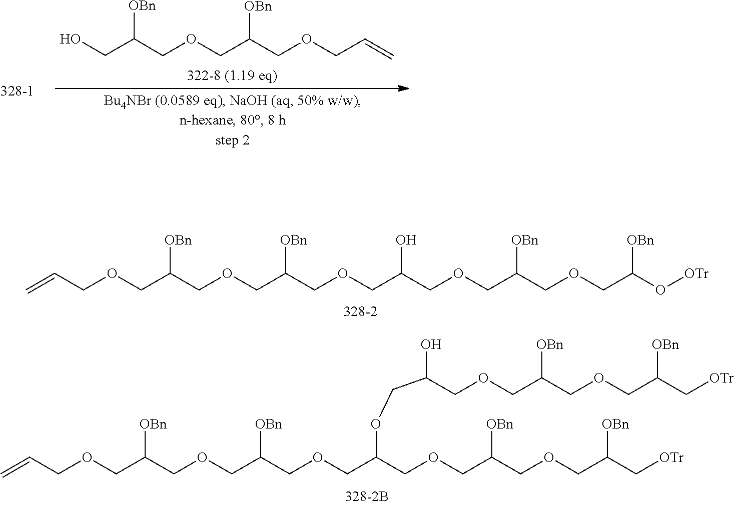

A solution of 322-8 (4.279 g, 11.08 mmol) and Bu₄NBr (177 mg, 0.548 mmol) in mixture solvent of n-hexane (20 mL) and NaOH (aq, 50% w/w, 20 mL) was stirred at 80° C. A mixture of 328-1 (6.0 g, 9.312 mmol) in n-hexane (8 mL) was dropped to the reaction mixture, and the resulting mixture was stirred at 80° C. for 8 hrs until most of 328-1 was consumed by TLC. The mixture was cooled down to r.t. and diluted with water (30 mL), then it was extracted with MTBE (70 mL×3). The organic parts were combined, dried over anhydrous Na₂SO₄, filtered and concentrated to dryness under reduced pressure. The crude product was purified with column chromatography (silica, 0-100% Ethyl acetate in petroleum ether) affording 328-2 (7.22 g, contains 328-2B) as a pale yellow oil. ¹H NMR (400 MHz, CDCl₃) δ 7.48-7.41 (m, 6H), 7.37-7.24 (m, 29H), 5.95-5.81 (m, 1H), 5.29-5.22 (m, 1H), 5.16 (dd, J=10.4, 1.7 Hz, 1H), 4.69-4.63 (m, 6H), 4.62-4.58 (m, 2H), 4.00-3.95 (m, 2H), 3.92-3.85 (m, 1H), 3.76-3.70 (m, 3H), 3.69-3.64 (m, 1H), 3.62-3.50 (m, 15H), 3.48-3.39 (m, 4H), 3.23 (d, J=5.2 Hz, 2H).

### Step 3

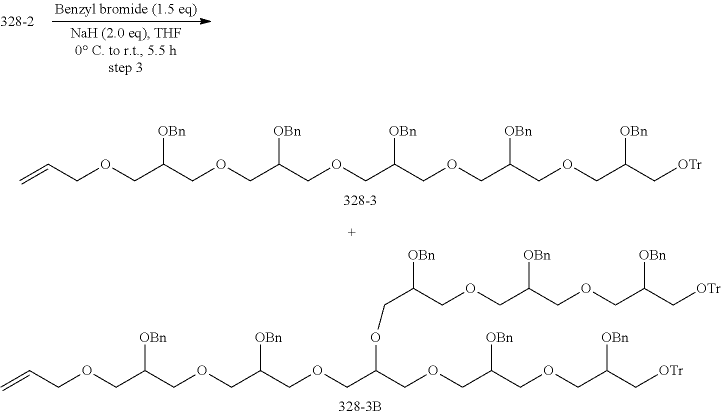

A mixture of NaH (560.5 mg, 14.012 mmol) in anhydrous THF (23.5 mL) was stirred in an ice bath, then a solution of 328-2 (7.22 g, 7.006 mmol, contains 328-2B) in anhydrous THF (13.2 mL) was added dropwise. The reaction mixture was stirred at this temperature for 20 mins, then benzyl bromide (1.786 g, 10.509 mmol) was added. The resulting mixture was allowed to warm to r.t. and stirred for 5 hrs until 328-2 was consumed by TLC. The reaction was quenched with saturated ammonium (20 mL) and extracted with ethyl acetate (40 mL×2). The organic phase was combined, dried over anhydrous Na₂SO₄, filtered and concentrated to dryness under reduced pressure to give the crude product, which was purified with column chromatography (silica, 0-80% Ethyl acetate in petroleum ether) affording 328-3 (5.13 g, 4.578 mmol, 65.3%) as a pale yellow oil and 328-3B (2.0 g, 1.133 mmol) a pale yellow oil. Purity=90%-95%. ¹H NMR (400 MHz, CDCl₃) δ 7.46-7.42 (m, 6H), 7.33-7.20 (m, 34H), 5.94-5.81 (m, 1H), 5.27-5.22 (m, 1H), 5.17-5.12 (m, 1H), 4.67-4.63 (m, 8H), 4.61 (s, 2H), 3.99-3.95 (m, 2H), 3.75-3.66 (m, 5H), 3.60-3.50 (m, 18H), 3.23 (d, J=5.2 Hz, 2H). ¹H NMR (400 MHz, CDCl₃) δ 7.46-7.40 (m, 13H), 7.34-7.25 (m, 27H), 7.24-7.15 (m, 25H), 5.93-5.81 (m, 1H), 5.26-5.21 (m, 1H), 5.14 (dd, J=10.4, 1.6 Hz, 1H), 4.67-4.65 (m, 2H), 4.64-4.61 (m, 8H), 4.60-4.57 (m, 4H), 3.99-3.93 (m, 2H), 3.75-3.63 (m, 10H), 3.59-3.55 (m, 6H), 3.54-3.46 (m, 20H), 3.21 (d, J=4.8 Hz, 4H).

### Step 4

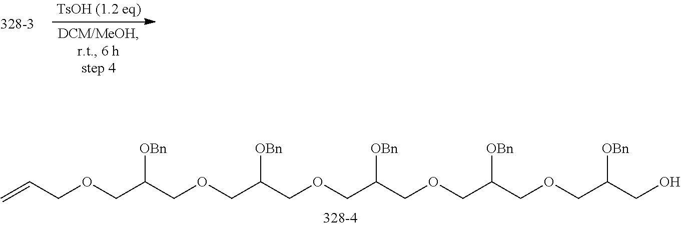 [SMILES: C=CCOCC(COCC(COCC(COCC(COCC(CO)OCc1ccccc1)OCc1ccccc1)OCc1ccccc1)OCc1ccccc1)OCc1ccccc1]

To a solution of 328-3 (3.6 g, 3.213 mmol) in a mixture solvent of DCM (22 mL) and MeOH (11 mL) was added TsOH·H₂O (733 mg, 3.855 mmol), the resulting mixture was stirred for 6 hrs at room temperature until 328-3 was consumed by TLC. The reaction was quenched with saturated NaHCO₃ (aq.), diluted with water (20 mL), and then extracted with DCM (40 mL×3). The organic phase was combined, dried over anhydrous Na₂SO₄, filtered and concentrated to dryness under reduced pressure to give the crude product, which was purified with column chromatography (silica, 0-60% ethyl acetate in petroleum ether) affording 328-4 (2.84 g, 23.233 mmol, 100%) as a colorless oil. Purity=90%-95%. ¹H NMR (400 MHz, CDCl₃) δ 7.38-7.26 (m, 25H), 5.96-5.84 (m, 1H), 5.27 (dq, J=17.2, 1.8 Hz, 1H), 5.17 (dq, J=10.4, 1.6 Hz, 1H), 4.73-4.63 (m, 10H), 4.63-4.58 (m, 1H), 3.99 (dt, J=5.6, 1.6 Hz, 2H), 3.78-3.71 (m, 5H), 3.67-3.63 (m, 2H), 3.62-3.54 (m, 18H).

### Step 5

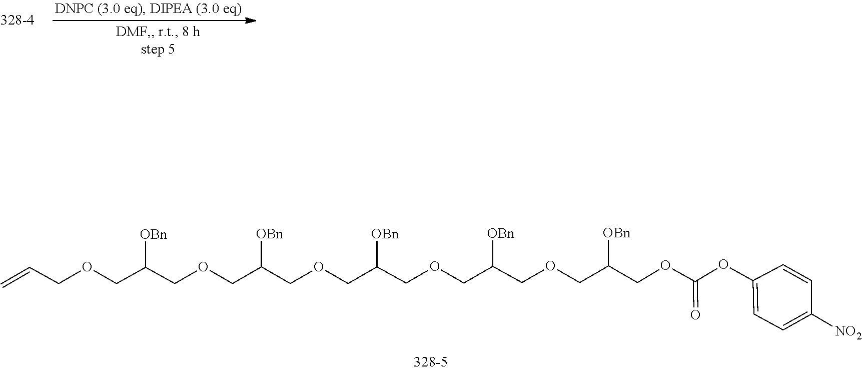 [SMILES: C=CCOCC(COCC(COCC(COCC(COCC(COC(=O)Oc1ccc([N+](=O)[O-])cc1)OCc1ccccc1)OCc1ccccc1)OCc1ccccc1)OCc1ccccc1)OCc1ccccc1]

To a solution of 328-4 (3.6 g, 3.213 mmol) in DMF (8 mL) was added DIPEA (353.2 mg, 2.733 mmol), followed by 4,4′-dinitrodiphenyl carbonate (831.1 mg, 2.732 mmol), then the resulting mixture was stirred at room temperature for 8 hrs until 328-4 was consumed detected by LCMS. The reaction solution was directly used in the next step without work-up procedure.

### Step 6

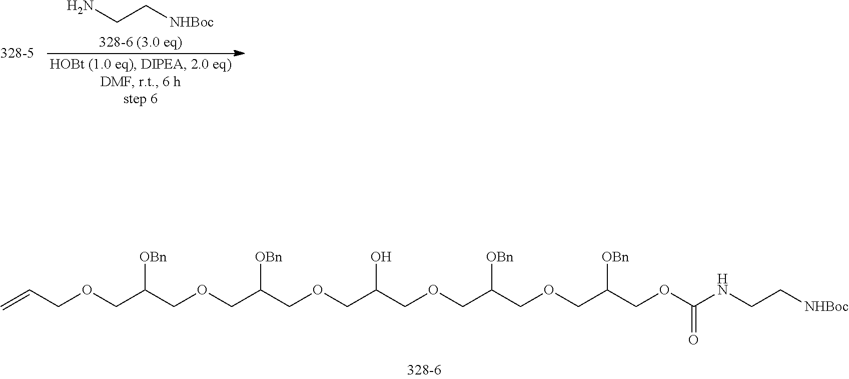 [SMILES: CC(C)(C)OC(=O)NCCN ; C=CCOCC(COCC(COCC(O)COCC(COCC(COC(=O)NCCNC(=O)OC(C)(C)C)OCc1ccccc1)OCc1ccccc1)OCc1ccccc1)OCc1ccccc1]

To the above reaction mixture was added HOBt (123 mg, 0.911 mmol), DIPEA (235.5 mg, 1.822 mmol) and 328-6 (437.6 mg, 2.733 mmol) successively, and the resulting mixture was stirred at room temperature for 6 hrs until 328-5 was consumed detected by LCMS. The reaction mixture was diluted with ethyl acetate (100 mL) and washed with saturated NaHCO₃ (aq, 30 mL×3), dried over anhydrous Na₂SO₄, filtered and concentrated to dryness under reduced pressure. The crude product was purified with column chromatography (silica, 0-90% ethyl acetate in petroleum ether) affording 328-6 (958 mg, 0.90 mmol, 98.7% over 2 steps) as a pale yellow oil. ¹H NMR (400 MHz, CDCl₃) δ 7.36-7.22 (m, 25H), 5.95-5.80 (m, 1H), 5.28-5.21 (m, 1H), 5.20-5.13 (m, 1H), 5.07 (s, 1H), 4.83 (s, 1H), 4.69-4.62 (m, 10H), 4.30-4.21 (m, 1H), 4.16-4.10 (m, 1H), 3.97 (dt, J=5.6, 1.6 Hz, 2H), 3.77-3.69 (m, 5H), 3.62-3.50 (m, 19H), 3.27-3.10 (m, 4H), 1.43 (s, 9H).

### Step 7

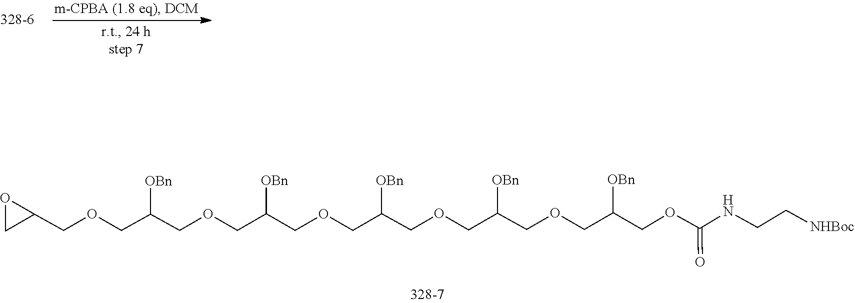 [SMILES: CC(C)(C)OC(=O)NCCNC(=O)OCC(COCC(COCC(COCC(COCC(COCC1CO1)OCc1ccccc1)OCc1ccccc1)OCc1ccccc1)OCc1ccccc1)OCc1ccccc1]

To a solution of 328-6 (958 mg, 0.90 mmol) in DCM (6 mL) was added a solution of m-CPBA (280 mg, 1.62 mmol) in DCM (6 mL) dropwise at room temperature. The resulting mixture was stirred at this temperature for 24 hours until 328-6 was consumed, and the reaction was quenched with saturated Na₂S₂O₃ (aq, 5 mL) and NaHCO₃ (aq., 5 mL). The reaction mixture was stirred for 30 mins, and then diluted with DCM (30 mL). The organic phase was washed with a mixture solution (10 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-100% ethyl acetate in petroleum ether) affording 328-7 (787 mg, 0.728 mmol, 80.9%) as a pale yellow oil. Purity=90%-95%.

### Step 8

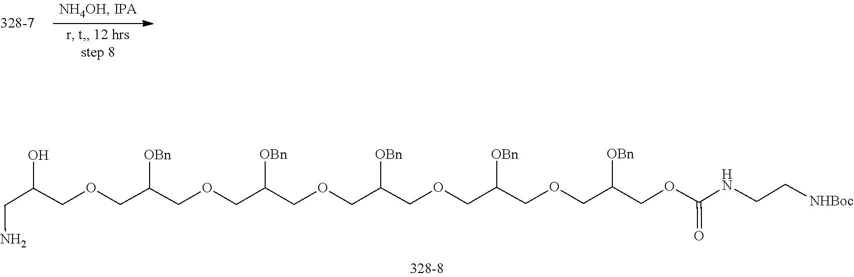 [SMILES: CC(C)(C)OC(=O)NCCNC(=O)OCC(COCC(COCC(COCC(COCC(COCC(O)CN)OCc1ccccc1)OCc1ccccc1)OCc1ccccc1)OCc1ccccc1)OCc1ccccc1]

To a solution of 328-7 (565 mg, 0.523 mmol) in isopropyl alcohol (43 mL) was added ammonia (43 mL) dropwise at room temperature, and the resulting mixture was stirred at this temperature for 12 hours until 328-7 was consumed. The reaction mixture was concentrated to dryness under reduced pressure to afford 328-8 (552.6 mg, 0.503 mmol, 96.3%) as a yellow oil, which was used in the next step without further purification. Purity=90%-95%.

### Step 9

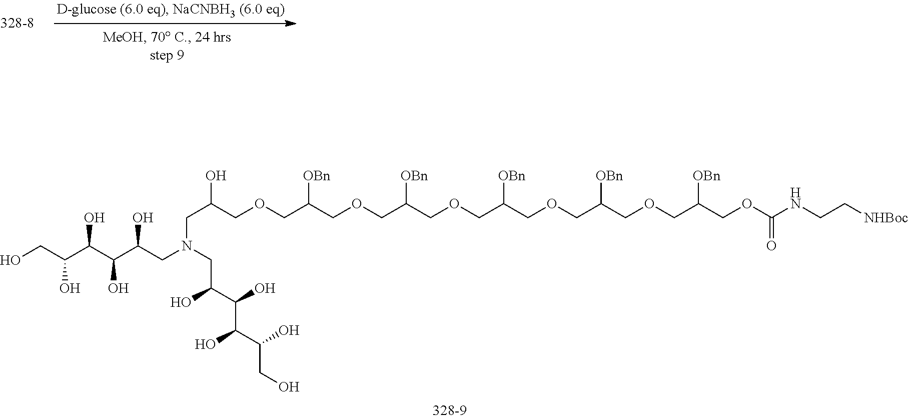 [SMILES: CC(C)(C)OC(=O)NCCNC(=O)OCC(COCC(COCC(COCC(COCC(COCC(O)CN(C[C@H](O)[C@@H](O)[C@H](O)[C@H](O)CO)C[C@H](O)[C@@H](O)[C@H](O)[C@H](O)CO)OCc1ccccc1)OCc1ccccc1)OCc1ccccc1)OCc1ccccc1)OCc1ccccc1]

A mixture of 328-8 (552.6 mg, 0.503 mmol), D-glucose (544.2 mg, 3.021 mmol) and NaCNBH₃ (189.8 mg, 3.021 mmol) in anhydrous MeOH (12 mL) was stirred at 70° C. for 24 hrs until most of 328-8 was consumed and 328-9 was detected by LCMS. The reaction mixture was cooled down to room temperature, filtered and concentrated under reduced pressure to give the crude product, which was purified by reverse phase liquid chromatography to give 328-9 (597 mg, 0.419 mmol, 83.2%) as a colorless oil. Purity=90%-95%. ¹H NMR (400 MHz, MeOH-d₄) δ 7.41-7.20 (m, 25H), 4.69-4.57 (m, 10H), 4.26-4.04 (m, 5H), 3.84-3.78 (m, 3H), 3.77-3.68 (m, 8H), 3.67-3.42 (m, 28H), 3.38-3.34 (m, 1H), 3.33-3.31 (m, 1H), 3.17-3.06 (m, 4H), 1.40 (s, 9H).

### Step 10

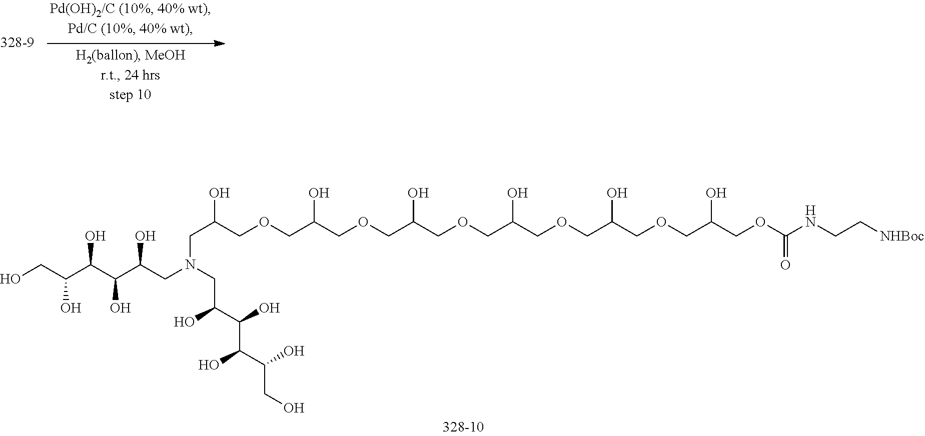 [SMILES: CC(C)(C)OC(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

A mixture of 328-9 (376 mg, 0.264 mmol), Pd(OH)₂/C (10%, 150 mg) and Pd/C (10%, 150 mg) in MeOH (30 mL) was stirred under hydrogen atmosphere (ballon) for 24 h at room temperature until 328-9 was completely converted into 328-10. The reaction mixture was filtered through a celite pad, and the filtrate was concentrated to dryness under reduced pressure to afford 328-10 (211.4 mg, 0.217 mmol, 82.1%) as an off-white solid, which was used in the next step without further purification.

### Step 11

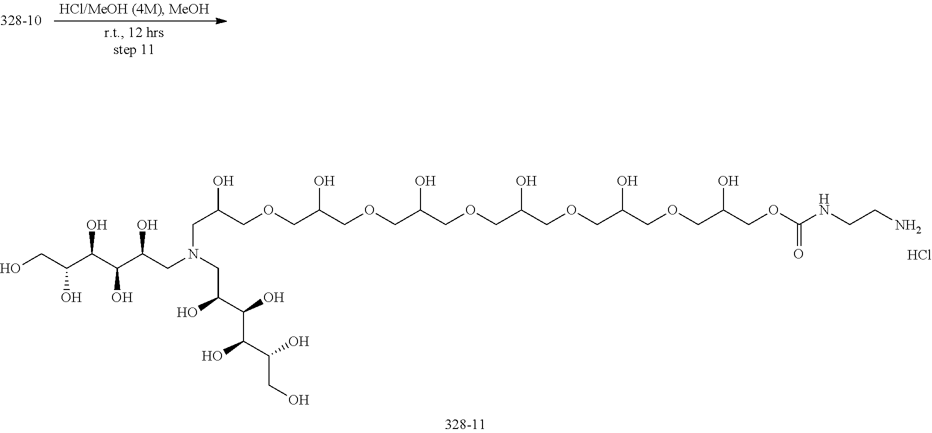 [SMILES: NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO ; Cl]

A solution of 328-10 (211.4 mg, 0.216 mmol) in HCl/MeOH (4M, 3 mL) and MeOH (3 mL) was stirred at room temperature for 12 hours until 328-10 was completely converted into 328-11. The reaction mixture was concentrated to dryness under reduced pressure to afford 328-11 (198.0 mg, 0.217 mmol, 100%) as an off-white solid, which was used in the next step without further purification.

### Step 12

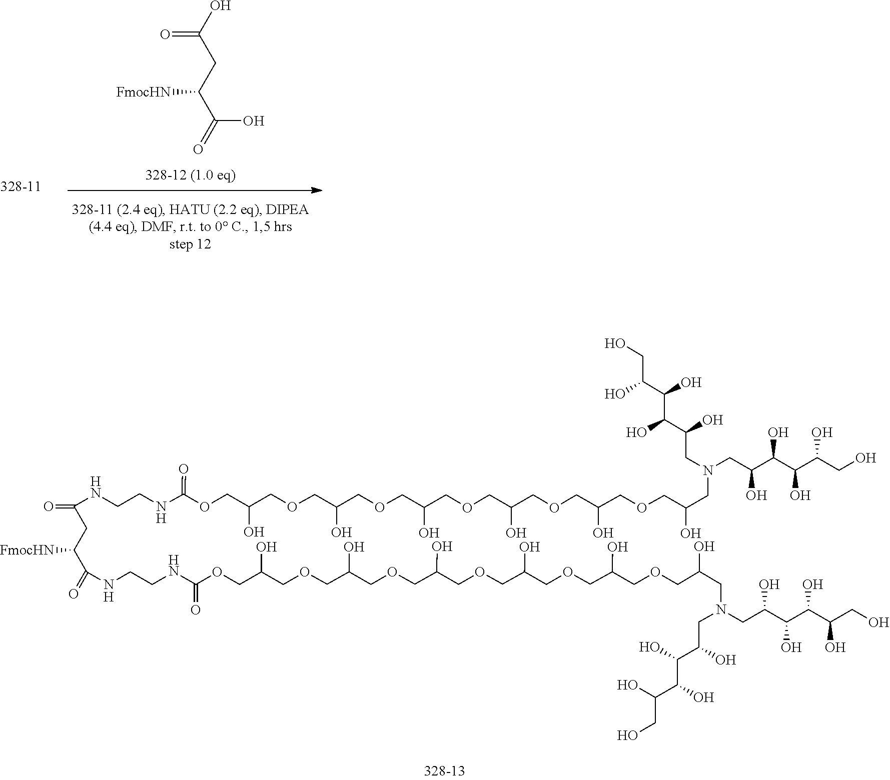

A solution of 328-12 (24.8 mg, 0.0698 mmol) and HATU (58.4 mg, 0.153 mmol) in anhydrous DMF (2 mL) was stirred at room temperature for 15 mins, then it was stirred in an ice bath. A solution of 328-11 (150 mg, 0.168 mmol) in anhydrous DMF (2 mL) was added dropwise, followed by DIPEA (39.7 mg, 0.307 mmol). The resulting mixture was stirred in the ice bath for 1 h until most of 328-12 was consumed. The reaction mixture was purified by reverse phase liquid chromatography to give 328-13 (97.5 mg, 0.0471 mmol, 67.4%) as a colorless oil. Purity=90%-95%.

### Step 13

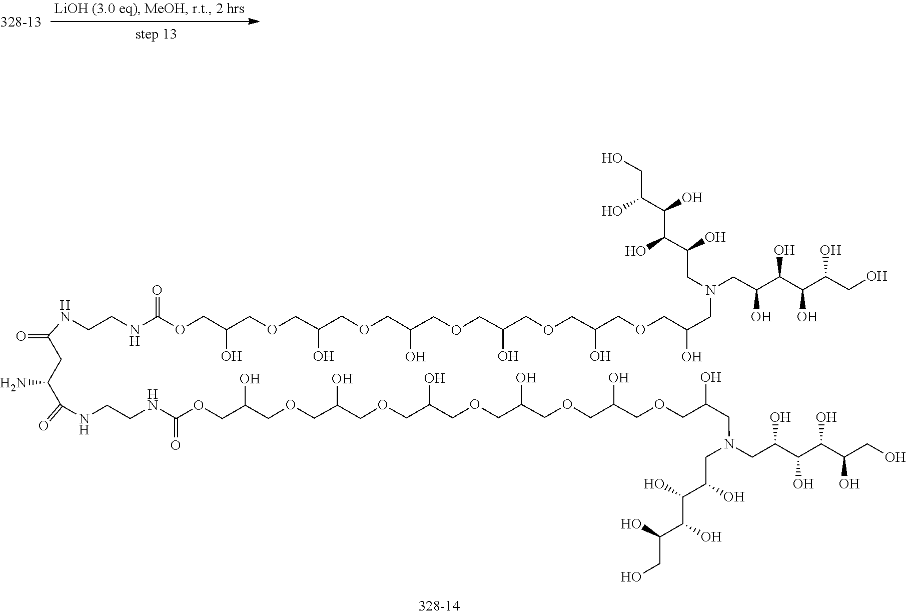 [SMILES: N[C@H](CC(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

To a solution of 328-13 (97.5 mg, 0.0471 mmol) in MeOH (3 mL) was added LiOH·H₂O (5.9 mg, 0.141 mmol), and the mixture was stirred at room temperature for 2 hrs until 328-13 was consumed. The reaction solution was neutralized with 1N HCl to pH=7, and concentrated under reduced pressure to give a crude product, which was dissolved in H₂O (10 mL) and washed with hexane (5 mL×3). The aqueous phase was concentrated to dryness under reduced pressure to afford 328-14 (87.0 mg, 0.047 mmol, 100%) as a colorless oil, which was used in the next step without further purification.

### Step 14

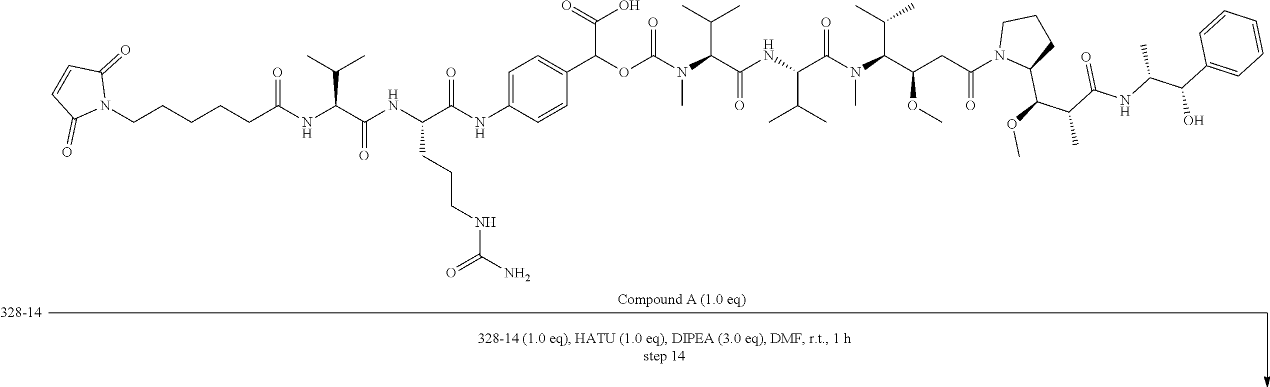

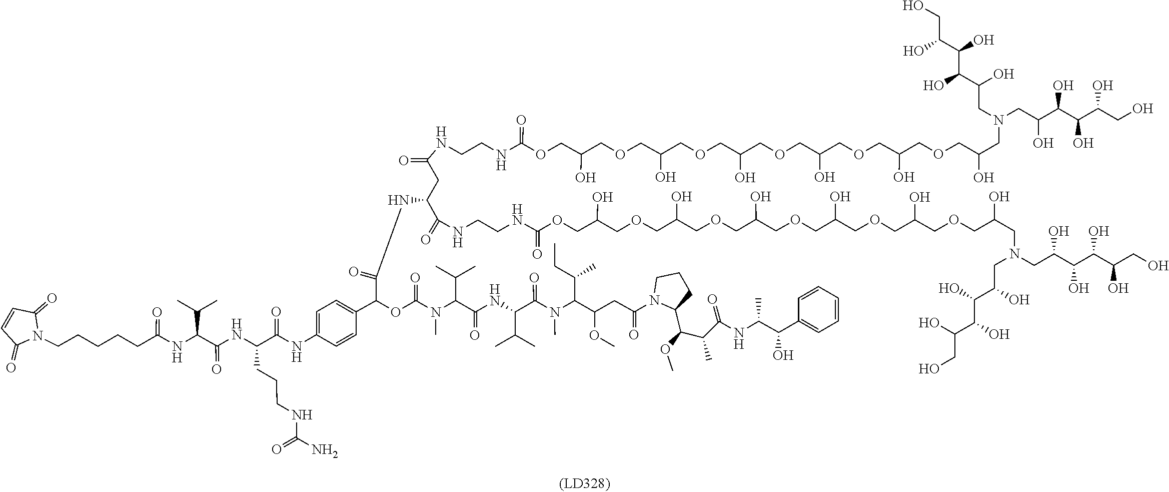 [SMILES: CC[C@H](C)C(C(CC(=O)N1CCC[C@H]1[C@H](OC)[C@@H](C)C(=O)N[C@H](C)[C@@H](O)c1ccccc1)OC)N(C)C(=O)[C@@H](NC(=O)C(C(C)C)N(C)C(=O)OC(C(=O)N[C@H](CC(=O)NCCNC(=O)OCC(O)COCC(O)COCC(O)COCC(O)COCC(O)COCC(O)CN(CC(O)[C@@H](O)[C@H](O)[C@H](O)CO)CC(O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)C(O)CO)c1ccc(NC(=O)[C@H](CCCNC(N)=O)NC(=O)[C@@H](NC(=O)CCCCCN2C(=O)C=CC2=O)C(C)C)cc1)C(C)C]

A solution of 328-14 (85 mg, 0.0460 mmol), Compound A (62 mg, 0.0460 mmol), HATU (17.2 mg, 0.0452 mmol) and DIPEA (17.9 mg, 0.139 mmol) in anhydrous DMF (4 mL) was stirred at room temperature for 1 h until Compound A was consumed. Then the reaction solution was purified by prep-HPLC to give LD328 (73.0 mg, 0.0229 mmol, 49.8%) as a white solid. LCMS, m/z=1595.97 (M/2+H)⁺, m/z=1064.29 (M/2+H)⁺. ¹H NMR (400 MHz, DMSO-d₆) δ 10.04 (s, 1H), 8.59-8.02 (m, 4H), 8.00-7.72 (m, 2H), 7.70-7.46 (m, 3H), 7.45-7.09 (m, 8H), 7.00 (s, 2H), 5.99 (s, 1H), 5.91-5.66 (m, 2H), 5.44 (s, 5H), 5.07-4.29 (m, 16H), 4.25-3.87 (m, 12H), 3.86-3.47 (m, 31H), 3.30-2.74 (m, 27H), 2.44-1.84 (m, 10H), 1.83-1.63 (m, 4H), 1.59-1.27 (m, 10H), 1.24-1.10 (m, 3H), 1.08-0.64 (m, 30H).

### Example 28: Preparation of a Conjugate of an Exemplary Drug-Linker and an Antibody

A solution of 10 mg/mL of anti-SLITRK6 (SLIT and NTRK-like protein 6) antibody hu1H2-03 in pH 7.1 PB 5 mM EDTA buffer is reduced by 10 mM TCEP at 25° C. for 120 minutes. 6.5 eq of 5 mM of LD328 (Linker Drug of Example 27) in DMA is added to the reduced antibody solution, and the resulting mixture is stirred at 25° C. for 120 min. The ADC is purified with PD-10 column to provide a conjugate of LD328 and hu1H2-03 (mAb1-LD328).

A conjugate of anti-SLITRK6 antibody Sirtratuzumab and vedotin (mAb2-vedotin) was prepared according to the specification of US20150238633 Å1. Sequences of the variable region and CDRs of anti-SLITRK6 antibodies are shown in Tables 1 and 2.

TABLE 1
Variable region sequence of anti-SLITRK6 antibodies

| Antibody | VH | VL |
|---|---|---|
| hu1H2-03 | evqlvesggglvqpggslrlscAAS | diqmtqspsslsasvgdrvtitc |
| | GFTFSDYGLHwvrqapgkglewvsY | RTSENIHSYLAwyqqkpgkSpQl |
| | ISSGSSTVYFADTLKGrftisrdna | lVyNAKTLADgvpsrfsgsgsgtd |
| | knslylqmnslraedtavyycTRGT | YtltisslqpedfGtFycQHFWTT |
| | WYFDVwgrgtlvtvss | SRTfgggtkveik |
| | (SEQ ID NO: 6) | (SEQ ID NO: 7) |
| Sirtratuzumab | QVQLVESGGGVVQPGRSLRLSCAASGF | DIVMTQSPLSLPVTPGEPASISCRSSQSL |
| | TFSSYGMHWVRQAPGKGLEWVAVIW | LLSHGFNYLDWYLQKPGQSPQLLIYLG |
| | YDGSNQYYADSVKGRFTISRDNSKNTL | SSRASGVPDRFSGSGSGTDFTLKISRVE |
| | FLQMHSLRAEDTAVYYCARGLTSGRY | AEDVGLYYCMQPLQIPWTFGQGTKVEIK |
| | GMDVWGQGTTVTVSS | (SEQ ID NO: 15) |
| | (SEQ ID NO: 14) | |

TABLE 2
CDR sequences of anti-SLITRK6 antibodies

| Anti-body | HCDR1 | HCDR2 | HCDR3 | LCDR1 | LCDR2 | LCDR3 |
|---|---|---|---|---|---|---|
| hu1H2-03 | DYGLH | YISSGSSTV | GTWYFDV | RTSENIHS | NAKTLAD | QHFWTTS |
| | (SEQ ID | YFADTLKG | (SEQ ID | YLA (SEQ | (SEQ ID | RT (SEQ ID |
| | NO: 8) | (SEQ ID | NO: 10) | ID NO: 11) | NO: 12) | NO: 13) |
| | | NO: 9) | | | | |
| Sirtratuzumab | GFTFSSYG | VAVIWYD | ARGLTSGR | RSSQSLLL | LGSSRA | MQPLQIPWT |
| | MH (SEQ | GSNQYYA | YGMDV | SHGFNYLD | (SEQ ID | (SEQ ID |
| | ID NO: 16) | DSVKG | (SEQ ID | (SEQ ID | NO: 20) | NO: 21) |
| | | (SEQ ID | NO: 18) | NO: 19) | | |
| | | NO: 17) | | | | |

### Example 29: In Vitro Study of a Conjugate of an Exemplary Drug-Linker and an Antibody on Cell Lines SW780 and CHP-212

The cytotoxicities of the two ADCs in Example 28 were evaluated with SLITRK6-expressing cell lines SW780 and CHP-212. One day prior to adding test article, mAb1-LD328 (8) or mAb2-vedotin (4) (used as a negative control), cells were harvested and plated into 96-well solid white flat bottom plates. The next day cells were exposed to the test article at concentrations from 666.67 to 0.034 nM. Plates were incubated at 37° C. for 144 h. After that, 40 μl Cell-titre Glo (CTG) per well was added to the plates with luciferase readings collected at 5 min after and analyzed by Microplate readers. All readings were normalized as percentage of viable cells in the untreated control wells and the IC₅₀ values were calculated by Prism software.

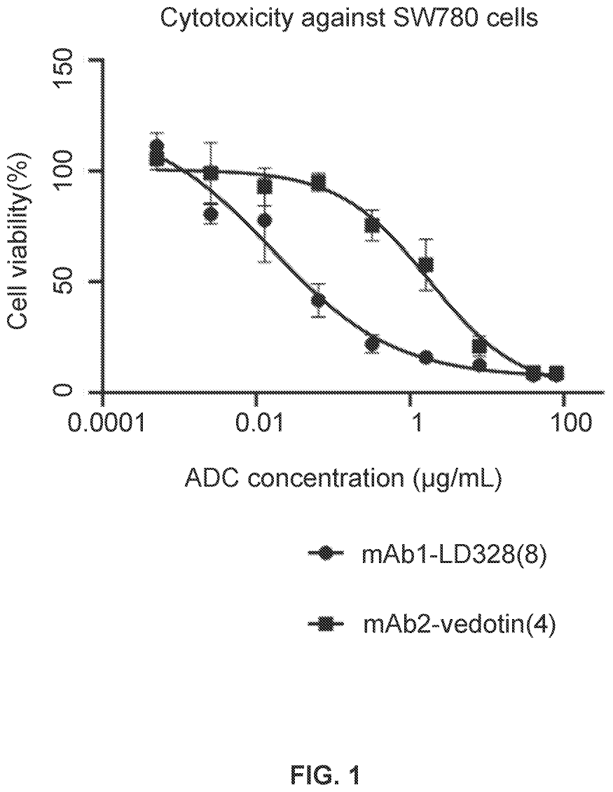

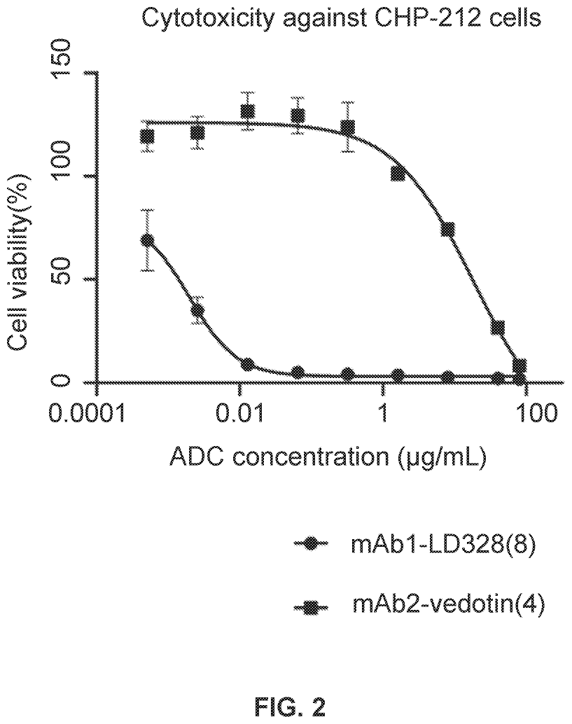

FIGS. 1 and 2 illustrate cytotoxicity of ADCs on cell lines SW780 and CHP-212, respectively. As shown in FIGS. 1 and 2, mAb-LD328 has a better in vitro cytotoxicity than mAb2-vedotin on SLITRK6-expressing cell lines.

### Example 30: In Vivo Study of a Conjugate of an Exemplary Drug-Linker and an Antibody on Cell Lines SW780

The in vivo anti-tumor activity of mAb1-LD328 (8) and mAb2-vedotin (4) were evaluated in tumor cell line SW780.

SW780 (Procell) tumor model was established by injecting 5×10⁶ cells suspended in 0.1 mL PBS mixed with Matrigel (1:1). 14 days after tumor inoculation, mice with average tumor size ˜125 mm³ were selected and assigned into 8 groups using stratified randomization (n=6 per group) based upon their tumor volumes. Mice were treated with a single (on day 0) intravenous injection of mAb1-LD328 (8) at 1.25 mg/kg or mAb2-vedotin (4) at 2.5 mg/kg.

The tumor size and body weight were measured. Animal body weight was monitored as an indirect measure of tolerability. No mice showed significant weight loss in any of the treatment groups. There were no morbidity and deaths during the treatment duration.

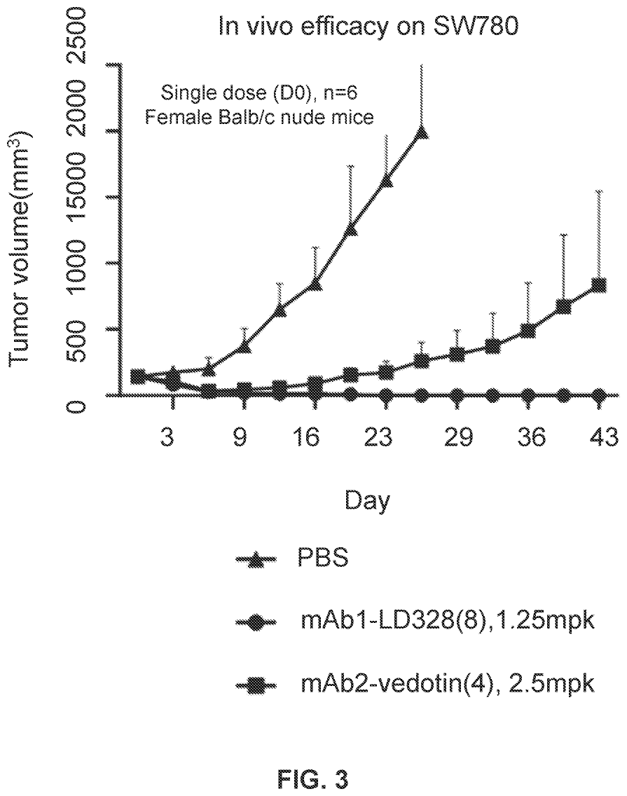

FIG. 3 illustrates efficacy of mAb1-LD328 (8) and mAb2-vedotin (4) in SW780 xenograft model. As shown in FIG. 3, mAb1-LD328 (8) had better in vivo efficacy than mAb2-vedotin (4) in SW780 xenograft model.

### Example 31: In Vivo Study of a Conjugate of an Exemplary Drug-Linker and an Antibody on Cell Line CHP-212

The in vivo anti-tumor activity of mAb1-LD328 (8) and mAb2-vedotin (4) were evaluated in tumor cell lines RT4.

RT4 (Procell) tumor model was established by injecting 5×10⁶ cells suspended in 0.1 mL PBS mixed with Matrigel (1:1). 14 days after tumor inoculation, mice with average tumor size ˜125 mm³ were selected and assigned into 8 groups using stratified randomization (n=6 per group) based upon their tumor volumes. Mice were treated with a single (on day 0) intravenous injection of mAb1-LD328 (8) at 1.25 mg/kg or mAb2-vedotin (4) at 2.5 mg/kg.

The tumor size and body weight were measured. Animal body weight was monitored as an indirect measure of tolerability. No mice showed significant weight loss in any of the treatment groups. There were no morbidity and deaths during the treatment duration.

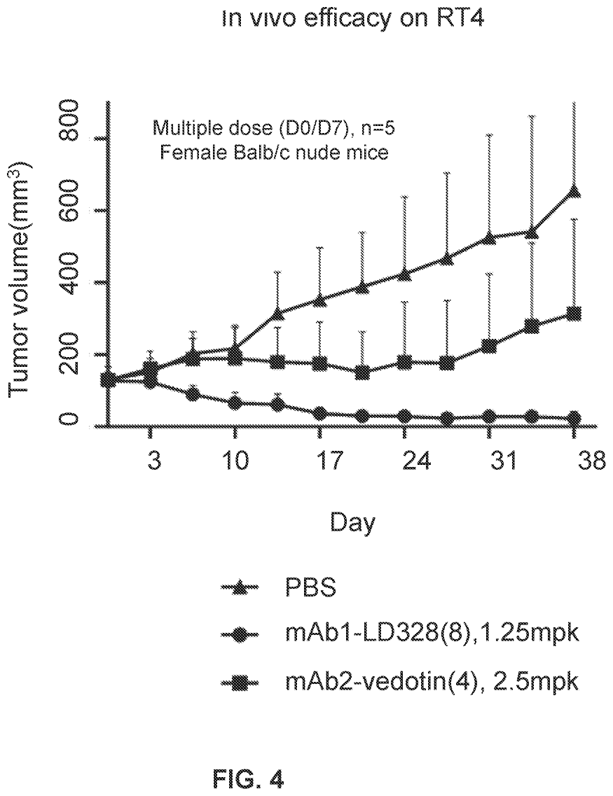

FIG. 4 illustrates efficacy of mAb1-LD328 (8) and mAb2-vedotin (4) in RT4 xenograft model. As shown in FIG. 3, mAb1-LD328 (8) had better in vivo efficacy than mAb2-vedotin (4) in RT4 xenograft model.

All of the U.S. patents, U.S. patent application publications, U.S. patent applications, foreign patents, foreign patent applications and non-patent publications referred to in this specification or the attached Application Data Sheet are incorporated herein by reference, in their entirety to the extent not inconsistent with the present description.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A Linker compound, comprising:
(a) a Linker unit having from 1 to 4 attachment sites for a Drug unit;
(b) an Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit, wherein the Polymer unit comprises the formula: 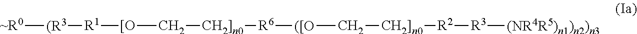
or a stereoisomer or salt thereof, wherein:
R⁰ is a functional group for attachment to a subunit of the Amino Acid unit;
each R¹ and R² are independently a bond or C₁-C₆ alkylene;
each R³ is independently selected from a bond, C₁-C₁₂ alkylene, —C(O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —C₁-C₁₂ alkylene-NR^{a}—C(O)—, —C₁-C₁₂ alkylene-C(O)—NR^{a}—C₁-C₁₂ alkylene-, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, —NR^{a}—C(O)—C₁-C₁₂ alkylene-C(O)—, —C(O)—NR^{a}—C₁-C₁₂ alkylene-(CH(OH))₁₋₈—C₁-C₁₂ alkylene-, —O—CH₂—CH₂, —O—C(O)—NR^{a}—C₁-C₁₂ alkylene, —O—CH₂—CH(OH)—C(O)—, —O—CH₂—CH(OH)—C(O)—NR^{a}—C₁-C₁₂ alkylene-, —CH(OH)—, —CH(OH)—C₁-C₁₂ alkylene-, C₁-C₁₂ alkylene-CH(OH)—, —CH(OH)—C(O)—, —CH(OH)—C(O)—NR^{a}—C₁-C₁₂ alkylene-, —CH(OH)—C₁-C₁₂ alkylene-NR^{a}—C(O)—C₁-C₁₂ alkylene-C(O)—NR^{a}—C₁-C₁₂ alkylene-, —NR^{a}—C(O)—C₁-C₁₂ alkylene-C(O)—NR^{a}—C₁-C₁₂ alkylene-, —CH(OH)—NR^{a}—C₁-C₁₂ alkylene-, —[C(O)—(CH₂)₁₋₈—NR^{a}]₁₋₈—, triazolyl, —C₁-C₁₂ alkylene-triazolyl-, —N(polyhydroxyl group)-, and —C(O)NR⁷R⁸, wherein one of R⁷ and R⁸ is H or C₁-C₁₂ alkylene and the other is C₁-C₁₂ alkylene, each R^{a} is independently selected from H, C₁₋₆ alkyl, and wherein any of the above alkylene groups may be substituted with —SO₃H;
each R⁴ and R⁵ are independently H, a polyhydroxyl group, a carboxyl-containing moiety, a substituted polyhydroxyl group, a —C(O)-polyhydroxyl group, a substituted —C(O)— polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
each R⁶ is selected from: 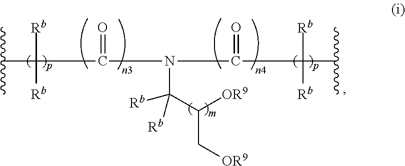
wherein:
each n³ and n⁴ are independently 0-1,
each R^{b} is independently H or C₁₋₆ alkyl,
each R⁹ is independently H, acetyl, —P(═O)(OH)₂, or —(CH₂)ᵥ—O—S(═O)₂(OH),
each p is independently 0-6,
m is 1-4,
each v is independently 1-6, and
n2 is 1; 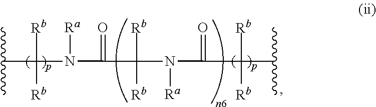
wherein:
each R^{a} is independently H or C₁₋₆ alkyl,
each R^{b} is independently H or C₁₋₆ alkyl,
n6 is 1-10,
each p is independently 0-6, and
n2 is 1; 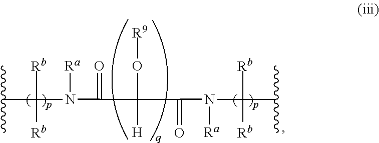
wherein:
each R^{a} is independently H or C₁₋₆ alkyl,
each R^{b} is independently H or C₁₋₆ alkyl,
each R⁹ is independently H, acetyl, —P(═O)(OH)₂, or —(CH₂)ᵥ—O—S(═O)₂(OH),
each p is independently 0-6,
q is 1-8,
each v is independently 1-6, and
n2 is 1; 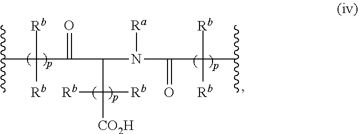
wherein:
each R^{a} is independently H or C₁₋₆ alkyl,
each R^{b} is independently H or C₁₋₆ alkyl,
each p is independently 0-6, and
n² is 1;
(v) —R¹⁰—[O—CH₂—CH₂]₁₋₈—R¹⁰—, wherein:
each R^{b} is independently H or C₁₋₆ alkyl,
each R¹⁰ is independently 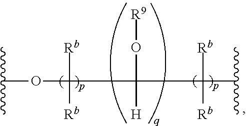
each p is independently 1-6,
each R⁹ is independently H, acetyl, —P(═O)(OH)₂, or —(CH₂)ᵥ—O—S(═O)₂(OH), and
q is 1-8;
n2 is 1; and
(vi) —N—(R¹—X—R²—)₂, wherein:
each X is independently —NR^{a}—C(O)— or —C(O)NR^{a}—, and
n² is 2; and
the wavy line (˜) indicates the attachment site of the Amino Acid unit to R;
each n⁰ is independently 2-26;
each n¹ is independently 1-6; and
n³ is 1-6.

2. A Linker compound, comprising:
(a) a Linker unit having from 1 to 4 attachment sites for a Drug unit;
(b) an Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit, wherein said Polymer unit comprises the formula: 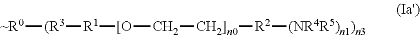
or a stereoisomer or salt thereof, wherein:
R⁰ is a functional group for attachment to a subunit of the Amino Acid unit;
each R¹ and R² are independently a bond or C₁-C₆ alkylene;
each R³ is independently —N(polyhydroxyl group)-, triazolyl, —C₁-C₁₂ alkylene-triazolyl-, 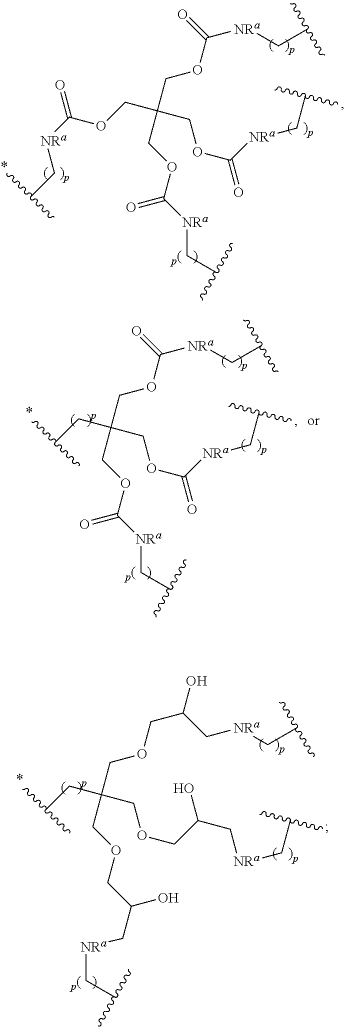
each R⁴ and R⁵ are independently H, a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R and R is not H;
each R^{a} is independently H or C₁₋₆ alkyl; 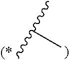
indicates the attachment site of R³ to R⁰
the wavy line 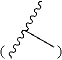
indicates the attachment site of the R³ to R¹;
each p is 1-6;
each n⁰ is independently 2-8;
each n¹ is independently 1-6; and
n³ is 1-6.

3. A Linker compound, comprising:
(a) a Linker unit having from 1 to 4 attachment sites for a Drug unit;
(b) an Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit, wherein said Polymer unit comprises the formula: 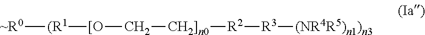
or a stereoisomer or salt thereof, wherein:
(i) R⁰ is a functional group for attachment to a subunit of the Amino Acid unit;
each R¹ and R² are independently a bond or C₁-C₆ alkylene;
R³ is —C(O)—;
R⁴ is H;
R⁵ is independently a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate;
the wavy line (˜) indicates the attachment site of the Amino Acid unit to R⁰;
n⁰ is independently 2-26;
n¹ is 1-6; and
n³ is 1-6;
(ii) R⁰ is —C(O)—;
R¹, R², and R³ are each a bond;
R⁴ and R⁵ are each independently H, a polyhydroxyl group, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
the wavy line (˜) indicates the attachment site of the Amino Acid unit to R⁰;
n⁰ is 6;
n¹ is 1-6; and
n³ is 1;
(iii) R⁰ is a functional group for attachment to a subunit of the Amino Acid unit;
R¹ and R² are each, independently, a bond or C₁-C₆ alkylene;
R³ is-NRₐ—C(O)—C₁-C₁₂ alkylene-C(O)—, wherein the alkylene is substituted with —SO₃H;
R^{a} is H or C₁₋₆ alkyl;
R⁴ and R⁵ are each independently H, a carboxyl-containing moiety, a polyhydroxyl group, a substituted polyhydroxyl group, a —C(O)-polyhydroxyl group, a substituted —C(O)— polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
the wavy line (˜) indicates the attachment site of the Amino Acid unit to R⁰;
each n⁰ is independently 1-26;
n¹ is 1-6; and
n³ is 1-6; or
(iv) R⁰ is 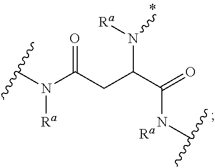
each R¹ is independently a bond or C₁-C₆ alkylene;
R² and R³ are each a bond;
R⁴ and R⁵ are each independently H, a polyhydroxyl group, a carboxyl-containing moiety, a substituted polyhydroxyl group, a —C(O)-polyhydroxyl group, a substituted —C(O)— polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
each R is independently H or C₁₋₆ alkyl;
the wavy line 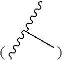
indicates the attachment site of R⁰ to the remainder of the Polymer unit;
the wavy line (˜*) indicates the attachment site of the Amino Acid unit to R⁰;
n⁰ is 1-8;
n¹ is 1-6; and
n³ is 2.

4. A Linker compound, comprising:
(a) a Linker unit having from 1 to 4 attachment sites for a Drug unit, said Linker unit comprising a moiety of formula: 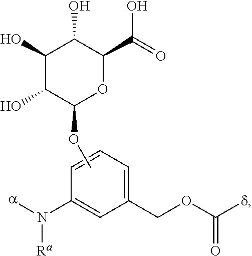
or a stereoisomer or salt thereof, wherein:
α—represents a direct or indirect attachment site to an Amino Acid unit;
δ—represents an attachment site to at least one of the Drug units or for a linking group attached to the at least one of the Drug units; and
R^{a} is H or C₁₋₆ alkyl;
(b) the Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit.

5. A Linker compound, comprising:
(a) a Linker unit having from 1 to 4 attachment sites for a Drug unit;
(b) an Amino Acid unit having from 1 to 12 amino acid subunits; and
(c) at least one Polar group attached to the Amino Acid unit, wherein the Polar group comprises a Polymer unit, optionally a Sugar unit, and optionally a Carboxyl unit, wherein said Polymer unit comprises:
(i) an optionally substituted polyamide comprising the formula 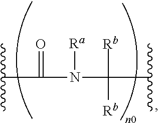
or a stereoisomer thereof, wherein each R^{a} is independently H or C₁₋₆ alkyl and each R^{b} is independently H or C₁₋₆ alkyl, and n⁰ is independently 2-26;
(ii) a substituted polyether comprising the formula 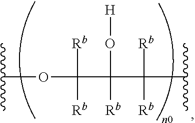
or a stereoisomer thereof, wherein each R^{b} is independently H or C₁₋₆ alkyl, and n⁰ is independently 2-26; or
(iii) combinations thereof.

6. The Linker compound of claim 4 or 5, wherein at least one Polar group attached to the Amino Acid unit comprises the formula: 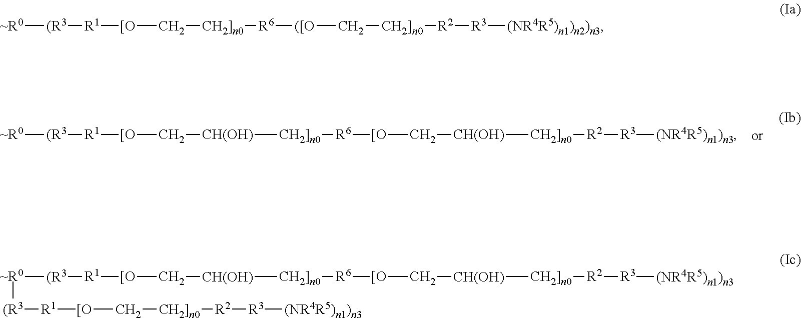
or a stereoisomer or salt thereof, wherein:
R⁰ is a functional group for attachment to a subunit of the Amino Acid unit;
each R¹ and R² are independently a bond or C₁-C₆ alkylene;
each R³ is independently selected from a bond, C₁-C₁₂ alkylene, —C(O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —C₁-C₁₂ alkylene-NR^{a}—C(O)—, —C₁-C₁₂ alkylene-C(O)—NR^{a}—C₁-C₁₂ alkylene-, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, —NR^{a}—C(O)—C₁-C₁₂ alkylene-C(O)—, —C(O)—NR^{a}—C₁-C₁₂ alkylene-(CH(OH))₁₋₈—C₁-C₁₂ alkylene-, —O—CH₂—CH₂, —O—C(O)—NR^{a}—C₁-C₁₂ alkylene, —O—CH₂—CH(OH)—C(O)—, —O—CH₂—CH(OH)—C(O)—NR^{a}—C₁-C₁₂ alkylene-, —CH(OH)—, —CH(OH)—C₁-C₁₂ alkylene-, C₁-C₁₂ alkylene-CH(OH)—, —CH(OH)—C(O)—, —CH(OH)—C(O)—NR^{a}—C₁-C₁₂ alkylene-, —CH(OH)—C₁-C₁₂ alkylene-NR^{a}—C(O)—C₁-C₁₂ alkylene-C(O)—NR^{a}—C₁-C₁₂ alkylene-, —NR^{a}—C(O)—C₁-C₁₂ alkylene-C(O)—NR^{a}—C₁-C₁₂ alkylene-, —CH(OH)—NR^{a}—C₁-C₁₂ alkylene-, —[C(O)—(CH₂)₁₈—NR^{a}]₁₋₈—, triazolyl, —C₁-C₁₂ alkylene-triazolyl-, and —C(O)NR⁷R⁸, wherein one of R⁷ and R⁸ is H or C₁-C₁₂ alkylene and the other is C₁-C₁₂ alkylene, each R^{a} is independently selected from H, C₁₋₆ alkyl, and wherein any of the above alkylene groups may be substituted with —SO₃H;
each R⁴ and R⁵ are independently H, a polyhydroxyl group, a carboxyl-containing moiety, a substituted polyhydroxyl group, a —C(O)-polyhydroxyl group, a substituted —C(O)— polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
each R⁶ is independently a bond or selected from: 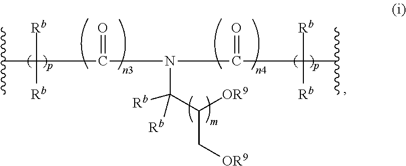
wherein:
each n³ and n⁴ are independently 0-1,
each R^{b} is independently H or C₁₋₆ alkyl,
each R⁹ is independently H, acetyl, —P(═O)(OH)₂, or —(CH₂)ᵥ—O—S(═O)₂(OH),
each p is independently 0-6,
m is 1-4, and
each v is independently 1-6, and
n2 is 1; 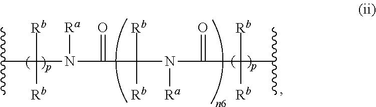
wherein:
each R^{a} is independently H or C₁₋₆ alkyl,
each R^{b} is independently H or C₁₋₆ alkyl,
n⁶ is 1-10, and
each p is independently 0-6, and
n² is 1; 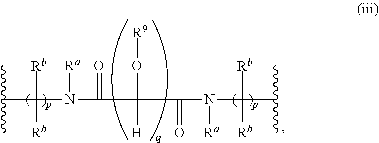
wherein:
each R^{a} is independently H or C₁₋₆ alkyl,
each R^{b} is independently H or C₁₋₆ alkyl,
each R⁹ is independently H, acetyl, —P(═O)(OH)₂, or —(CH₂)ᵥ—O—S(═O)₂(OH),
each p is independently 0-6, and
q is 1-8,
each v is independently 1-6, and
n² is 1; 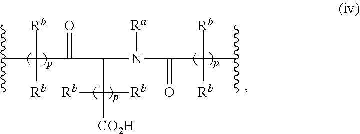
wherein:
each R^{a} is independently H or C₁₋₆ alkyl,
each R^{b} is independently H or C₁₋₆ alkyl, and
each p is independently 0-6, and
n² is 1;
(v) —R¹⁰—[O—CH₂—CH₂]₁₋₈—R¹⁰—, wherein:
each R^{b} is independently H or C₁₋₆ alkyl,
each R⁰ is independently 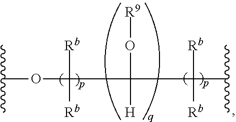
each p is independently 1-6, and
q is 1-8; and
(vi) —N—(R¹—X—R²—[O—CH₂—CH₂]ₙ₀—R²—R³—(NR⁴R⁵)ₙ₁)₂, wherein:
each X is independently —NR^{a}—C(O)— or —C(O)NR^{a}—, and
n² is 2; and
the wavy line (˜) indicates the attachment site of the Amino Acid unit to R⁰;
each n⁰ is independently 2-26;
n¹ is 0-6, and when n¹ is 0 then R³ is —OH or —C(O)OR^{b}, wherein R is independently H or C₁₋₆ alkyl; and
n³ is 1-6.

7. The Linker compound of any one of claim 1 or 4-6, wherein each R³ is independently selected from a bond, —C(O)—, —NR^{a}—C(O)—C₁-C₁₂ alkylene-C(O)—, —C(O)—NR^{a}—C, —C₁₂ alkylene-(CH(OH))₁₋₈—C₁-C₁₂ alkylene-, —O—CH₂—CH(OH)—C(O)—, —O—CH₂—CH(OH)—C(O)—NR^{a}—C₁-C₁₂ alkylene-, —CH(OH)—, —CH(OH)—C₁-C₁₂ alkylene-, C₁-C₁₂ alkylene-CH(OH)—, —CH(OH)—C(O)—, —CH(OH)—C(O)—NR^{a}—C₁-C₁₂ alkylene-, —CH(OH)—C₁-C₁₂ alkylene-NR^{a}—C(O)—C₁-C₁₂ alkylene-C(O)—NR^{a}—C₁-C₁₂ alkylene-, —NR^{a}—C(O)—C₁-C₁₂ alkylene-C(O)—NR^{a}—C₁-C₁₂ alkylene-, —CH(OH)—NR^{a}—C₁-C₁₂ alkylene-, —[C(O)—(CH₂)₁₋₈—NR^{a}]₁₋₈—, triazolyl, and —C₁-C₁₂ alkylene-triazolyl-, —N(polyhydroxyl group)-, each R^{a} is independently selected from H, C₁₋₆ alkyl; and wherein any of the above alkylene groups may be substituted with —SO₃H.

8. The Linker compound of any one of claims 1-3 or 5-7, wherein the Linker unit comprises a moiety selected from: 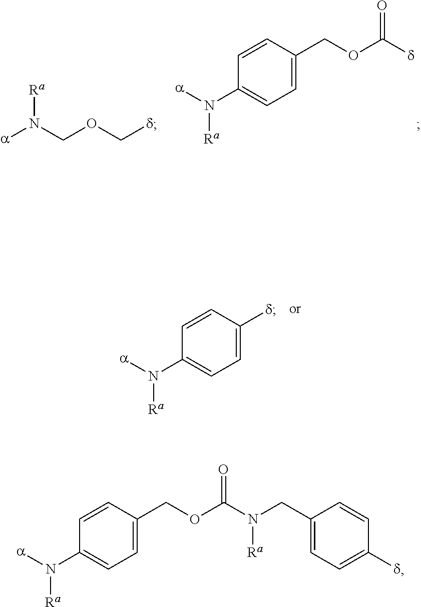
or a stereoisomer or salt thereof, wherein:
α—represents a direct or indirect attachment site to the Amino Acid unit or;
δ—represents an attachment site to at least one of the Drug units or an attachment site to a linking group attached to the at least one of the Drug units; and
R^{a} is H or C₁₋₆ alkyl.

9. The Linker compound of any one of claims 1-8, wherein the at least one Polar group comprises at least one Sugar unit having the following formula: 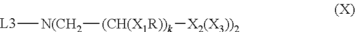
or a stereoisomer or salt thereof, wherein:
each X₁ is independently selected from NH or O;
each R is independently selected from hydrogen, acetyl, a monosaccharide, a disaccharide, and a polysaccharide;
each X₂ is independently selected from CH₂ and C(O);
each X₃ is independently selected from H, OH and OR;
k is 1 to 10; and
L3 is a point of attachment to the remainder of the Polar group.

10. The Linker compound of any one of claims 1-8, wherein the at least one Polar group comprises at least one Sugar unit having one of the following structures (XII) or (XIII): 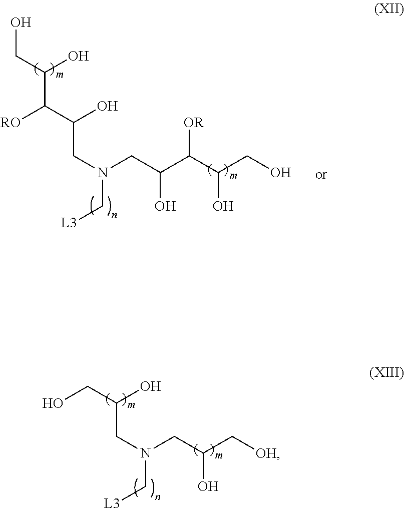
or a stereoisomer or salt thereof, wherein:
each R is independently selected from hydrogen, a monosaccharide, a disaccharide and a polysaccharide;
m is 1 to 8; and
n is 0 to 4.

11. The Linker compound of any one of claims 4-10, comprising a Polar group having a formula selected from: 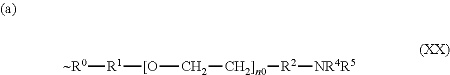
or a stereoisomer or salt thereof, wherein:
R⁰ is a functional group for attachment to a subunit of the Amino Acid unit;
R¹ and R² are each, independently, a bond or C₁-C₃ alkylene;
R⁴ and R⁵ are each independently selected from a H, polyhydroxyl group, substituted polyhydroxyl group, —C(O)-polyhydroxyl group, substituted —C(O)-polyhydroxyl group, a chelator, and —C(O)—R, where R is a Sugar unit of formula (XII) or (XIII), wherein both R⁴ and R⁵ are not H; and
n⁰ is 2 to 26; 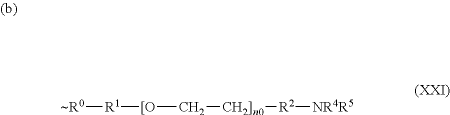
or a stereoisomer or salt thereof, wherein:
R⁰ is a functional group for attachment to a subunit of the Amino Acid unit;
R¹ and R² are each, independently, a bond or C₁-C₃ alkylene;
one of R⁴ and R⁵ is selected from a H, polyhydroxyl group, substituted polyhydroxyl group, —C(O)-polyhydroxyl group, substituted —C(O)-polyhydroxyl group, a chelator, and —C(O)—R, where R is a Sugar unit of formula (XII) or (XIII), and the other of R⁴ and R⁵ is a polyethylene glycol, optionally having 1 to 24 ethylene glycol subunits, wherein both R⁴ and R⁵ are not H; and
n⁰ is 2 to 26; 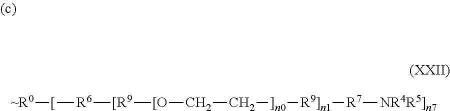
or a stereoisomer or salt thereof, wherein:
R⁰ is a functional group for attachment to a subunit of the Amino Acid unit;
R⁶ and R⁷ are each, independently, selected from a bond, C₁-C₁₂ alkylene, —NH—C₁-C₁₂ alkylene, —C₁-C₁₂ alkylene-NH—, —C₁-C₁₂ alkylene-N(CH₃)—, —C(O)—C₁-C₁₂ alkylene, —C₁-C₁₂ alkylene-C(O)—, —NH—C₁-C₁₂ alkylene-C(O)— and —C(O)—C₁-C₁₂ alkylene-NH—;
one of R⁴ and R³ is selected from a H, polyhydroxyl group, substituted polyhydroxyl group, —C(O)-polyhydroxyl group, substituted —C(O)-polyhydroxyl group, a chelator, and —C(O)—R, where R is a Sugar unit of formula (XII) or (XIII); and the other of R⁴ and R⁵ is selected from H, polyhydroxyl group, substituted polyhydroxyl group, —C(O)-polyhydroxyl group, substituted —C(O)-polyhydroxyl group, a chelator, and —C(O)—R, where R is a Sugar unit of formula (XII) or (XIII), and polyethylene glycol, optionally having 1 to 24 ethylene glycol subunits, wherein both R⁴ and R⁵ are not H;
each R⁹ is independently selected from a bond, —C(O)—, —NH—, —C(O)—C₁-C₆ alkylene-, —NH—C₁-C₆ alkylene-, —C₁-C₆ alkylene-NH—, —C₁-C₆ alkylene-C(O)—, —NH(CO)—C₁-C₆alkylene-, —N(CH₃)—(CO)—C₁-C₆alkylene-, —NH(CO)NH—, and triazole;
n0 is 2 to 26;
n1 is 1 to 4; and
n7 is 1 to 4; 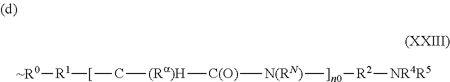
or a stereoisomer or salt thereof, wherein:
R⁰ is a functional group for attachment to a subunit of the Amino Acid unit;
R¹ is a bond, C₁-C₃ alkylene, —C₁-C₃alkylene-[O—CH₂—CH₂-]ₙ₀, —[CH₂—CH₂—O]ₙ₀—C₁-C₃alkylene- or —C₁-C₃ alkylene-[O—CH₂—CH₂—]ₙ₀—C(O)—;
R² is C₁-C₃ alkylene, —C₁-C₃alkylene-[O—CH₂—CH₂—]ₙ₀, —[CH₂—CH₂—O]ₙ₀—C₁-C₃alkylene- or —C₁-C₃ alkylene-[O—CH₂—CH₂—]ₙ₀—C(O)—;
each R^{α} is independently H or —R²—NR⁴R⁵;
each R^{N} is independently H, C₁-C₆ alkyl or —R²—NR⁴R⁵;
R⁴ and R⁵ are each independently selected from a H, polyhydroxyl group, substituted polyhydroxyl group, —C(O)-polyhydroxyl group, substituted —C(O)-polyhydroxyl group, a chelator, and —C(O)—R, where R is a Sugar unit of formula (XII) or (XIII), wherein both R⁴ and R⁵ are not H; and
each n⁰ is independently 2 to 26; 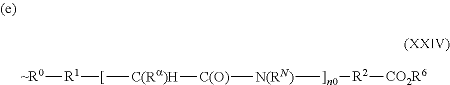
or a stereoisomer or salt thereof, wherein:
R⁰ is a functional group for attachment to a subunit of the Amino Acid unit;
R¹ is a bond, C₁-C₃ alkylene, or —C₁-C₃ alkylene[O—CH₂—CH₂—]ₙ₀;
R² is C₁-C₃ alkylene, or —C₁-C₃ alkylene[O—CH₂—CH₂—]ₙ₀;
each R^{α} is independently H or —R²—NR⁴R⁵;
each R^{N} is independently H, C₁-C₆ alkyl or —R²—NR⁴R⁵;
R⁴ and R⁵ are each independently selected from a H, polyhydroxyl group, substituted polyhydroxyl group, —C(O)-polyhydroxyl group, substituted —C(O)-polyhydroxyl group, a chelator, and —C(O)—R, where R is a Sugar unit of formula (XII) or (XIII), wherein both R⁴ and R⁵ are not H;
R⁶ is H or C₁-C₄ alkyl; and
each n⁰ is independently 2 to 26,
with the proviso that at least one R^{α} or R^{N} is —R²—NR⁴R⁵; or 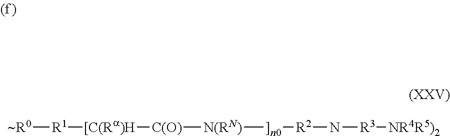
or a stereoisomer or salt thereof, wherein:
R⁰ is a functional group for attachment to a subunit of the Amino Acid unit;
R¹ and R² are each, independently, a bond, C₁-C₃ alkylene, or
—C₁-C₃alkylene-[O—CH₂—CH₂-]ₙ₀;
each R^{α} is independently H or —R²—NR⁴R⁵;
each R^{N} is independently H or C₁-C₆ alkyl;
each R³ is independently C₁-C₆ alkylene;
R⁴ and R⁵ are each independently selected from a H, polyhydroxyl group, substituted polyhydroxyl group, —C(O)-polyhydroxyl group, substituted —C(O)-polyhydroxyl group, a chelator, and —C(O)—R, where R is a Sugar unit of formula (XII) or (XIII), wherein both R⁴ and R⁵ are not H; and
each n0 is independently 2 to 26.

12. The Linker compound of any one of claims 1-11, wherein R⁴ and R⁵ are each independently selected from H and polyhydroxyl group, and wherein at least one of R⁴ and R⁵ is not H.

13. The Linker compound of claim 11 or 12, wherein the polyhydroxyl group is a linear monosaccharide, optionally selected from a C6 or C5 sugar, sugar acid or amino sugar.

14. The Linker compound of claim 13, wherein:
the C6 or C5 sugar is selected from glucose, ribose, galactose, mannose, arabinose, 2-deoxyglucose, glyceraldehyde, erythrose, threose, xylose, lyxose, allose, altrose, gulose, idose, talose, aldose, and ketose;
the sugar acid is selected from gluconic acid, aldonic acid, uronic acid and ulosonic acid; or
the amino sugar is selected from glucosamine, N-acetyl glucosamine, galactosamine, and N-acetyl galactosamine.

15. The Linker compound of any one of claims 1 to 14, comprising a Polar group selected from the following, or a stereoisomer or salt thereof: 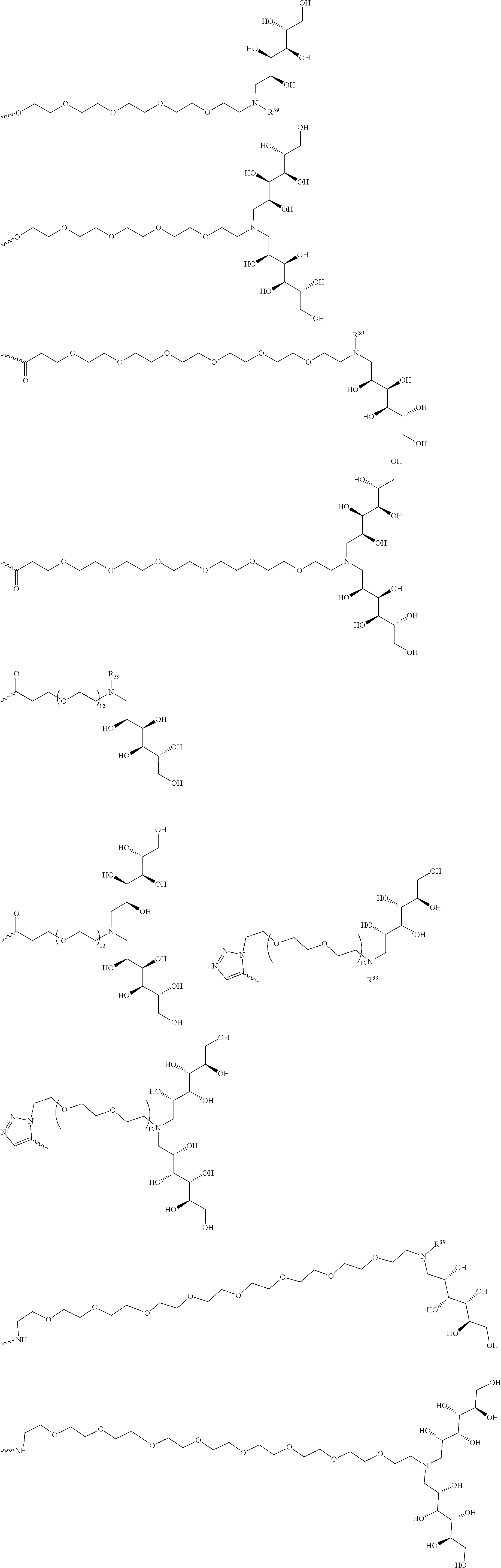 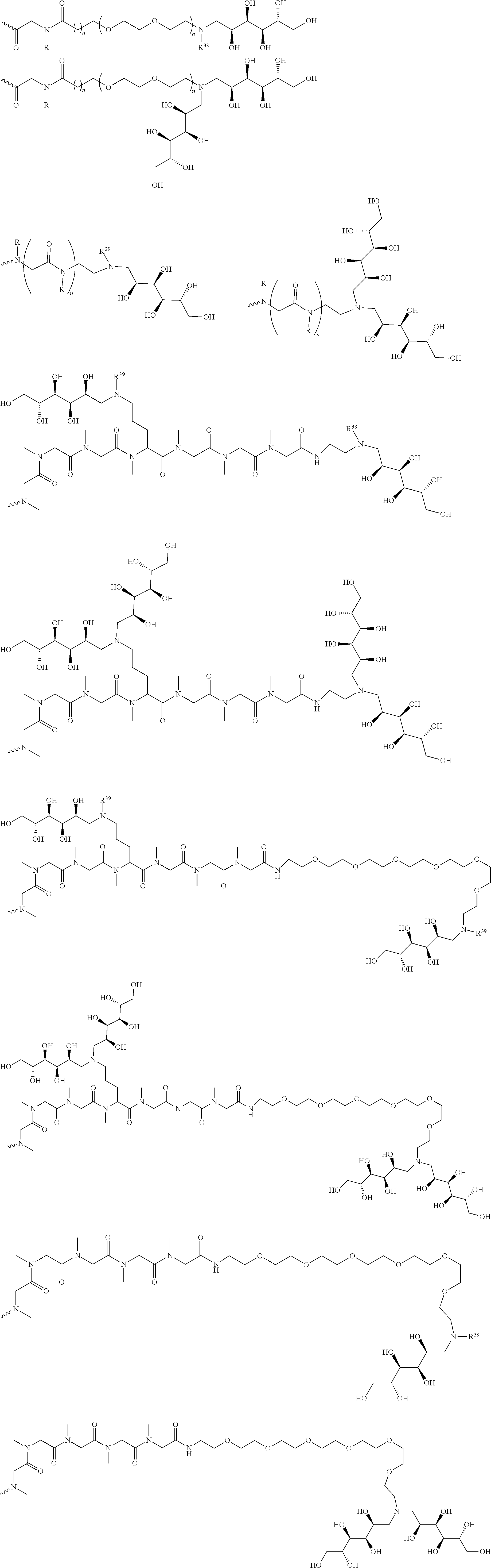 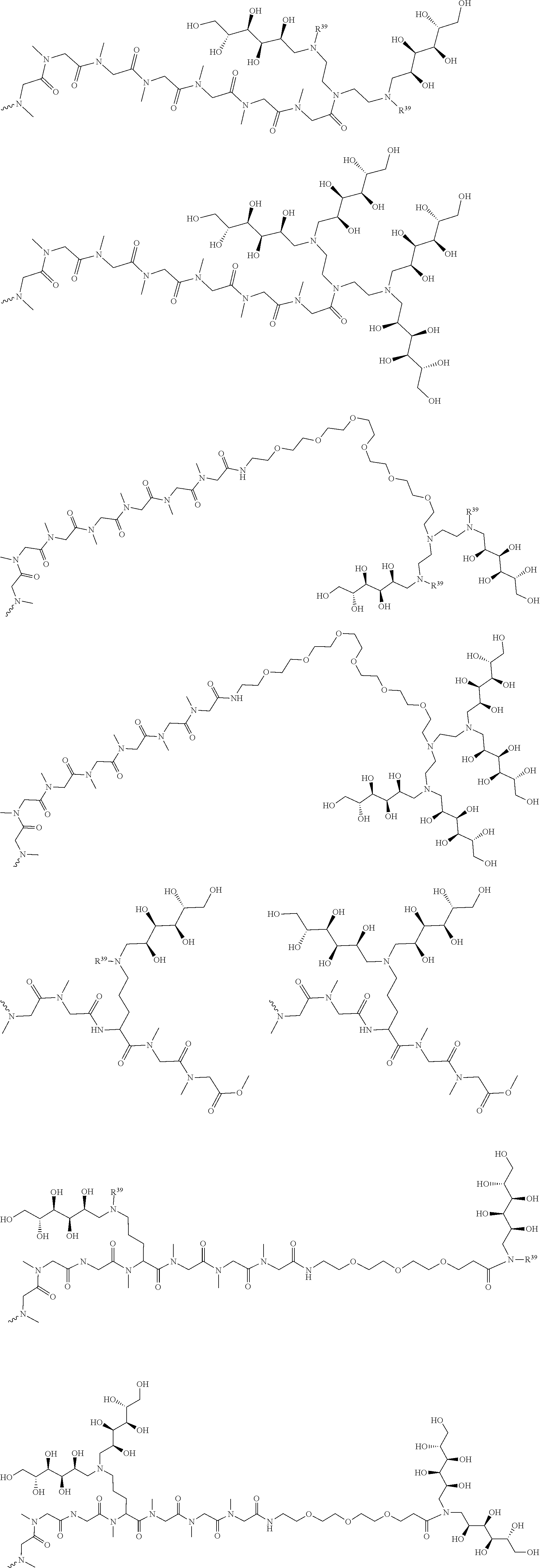 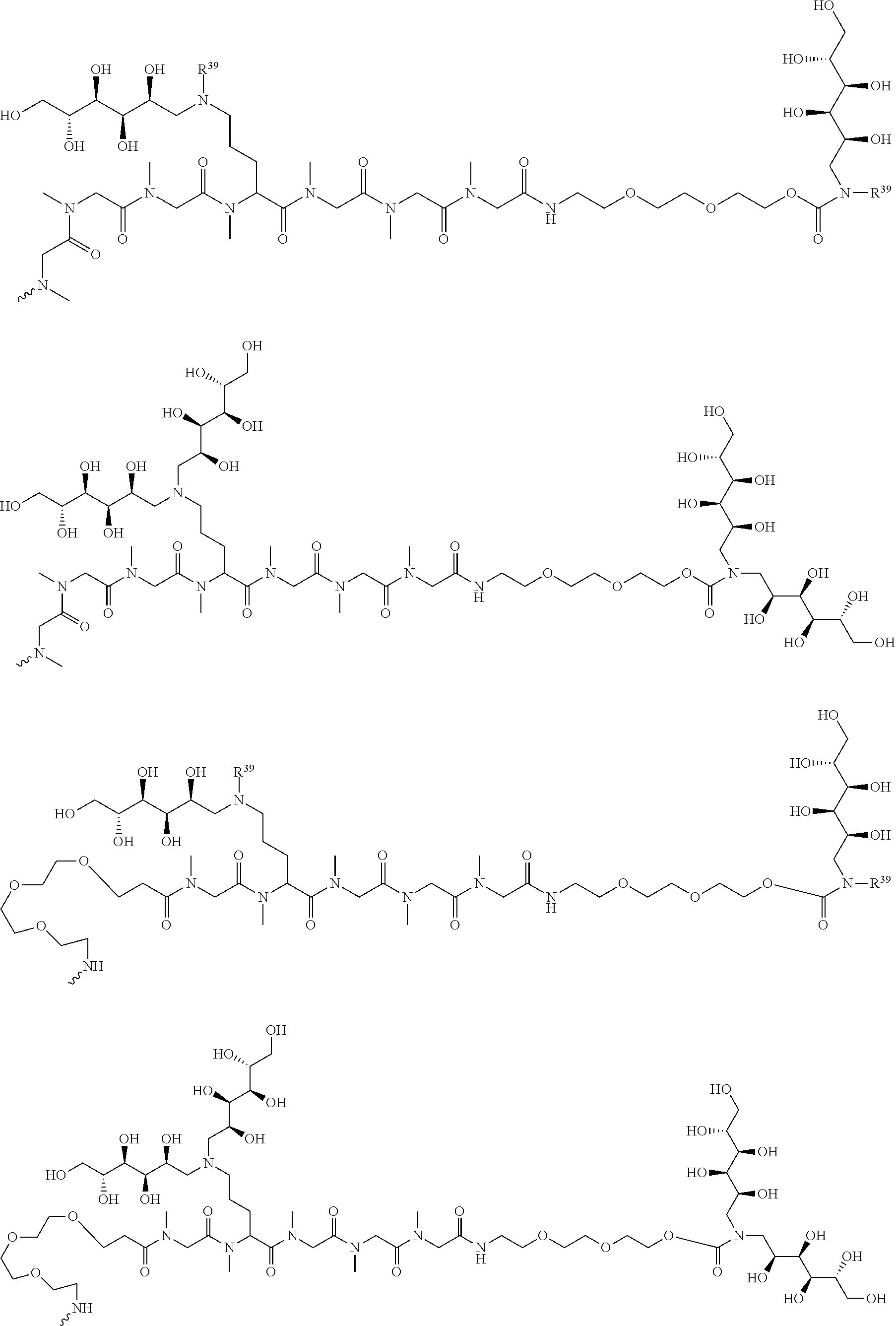 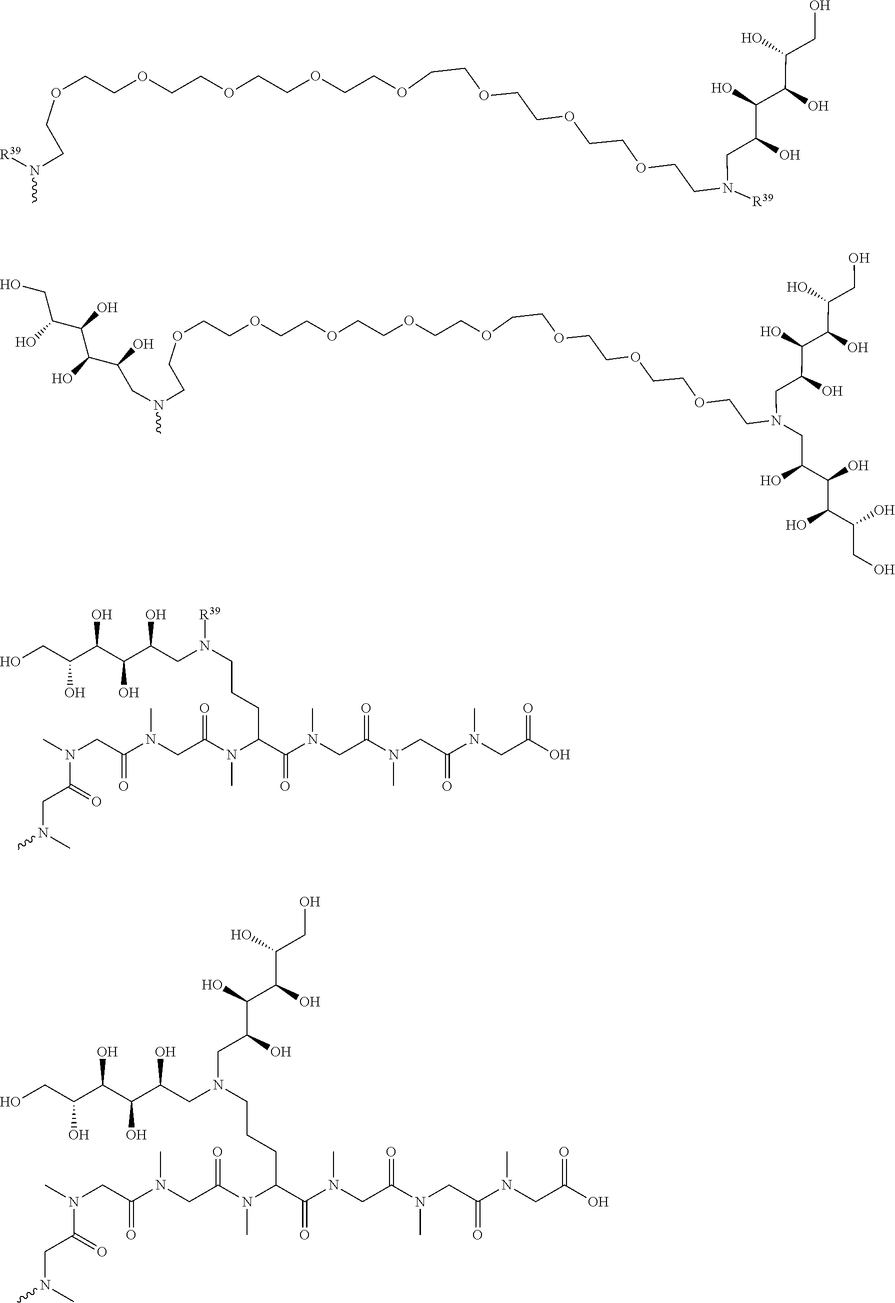
wherein each R is independently H or alkyl; each R³⁹ is independently selected from H, a linear monosaccharide and polyethylene glycol, optionally having from 1 to 24 ethylene glycol subunits; each n independently is 1-12; and the wavy line is an attachment to the Amino Acid unit.

16. The Linker compound of any one of claims 1-2 or 4-11, wherein one of R⁴ and R⁵ is a linear monosaccharide and the other is a cyclic monosaccharide.

17. The Linker compound of claim 16, wherein —(NR⁴R⁵) is selected from the following, or a stereoisomer or salt thereof: 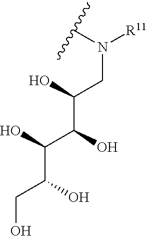
wherein R¹¹ is a cyclic monosaccharide.

18. The Linker compound of claim 16, comprising a Polar group selected from the following, or a stereoisomer or salt thereof: 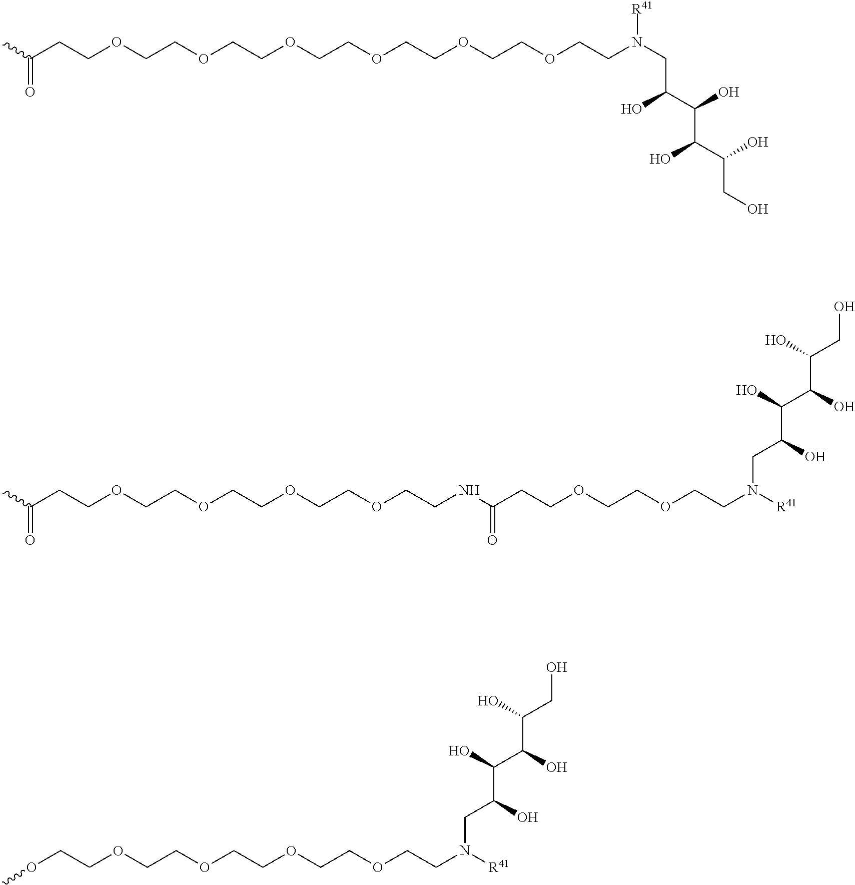
wherein R⁴¹ is a cyclic monosaccharide; and the wavy line is an attachment to the Amino Acid unit.

19. The Linker compound of any one of claims 1-2 or 4-11, wherein R⁴ and R⁵ are independently a polyhydroxyl selected from a cyclic monosaccharide, disaccharide and polysaccharide.

20. The Linker compound of claim 19, wherein —(NR⁴R⁵) is selected from the following, or a stereoisomer or salt thereof: 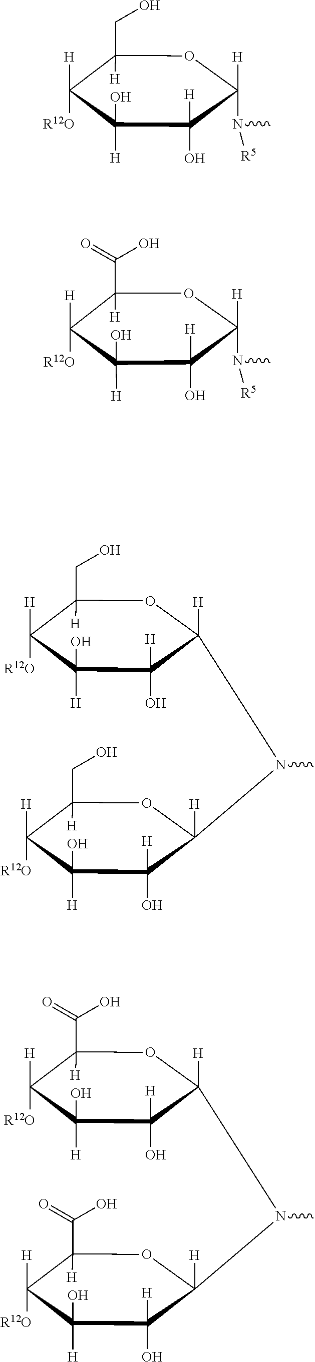
wherein each R¹² is selected from H and a monosaccharide, a disaccharide, or a polysaccharide; and R⁵ is selected from a cyclic monosaccharide, disaccharide, or polysaccharide.

21. The Linker compound of claim 19, comprising a Polar group selected from the following, or a stereoisomer or salt thereof: 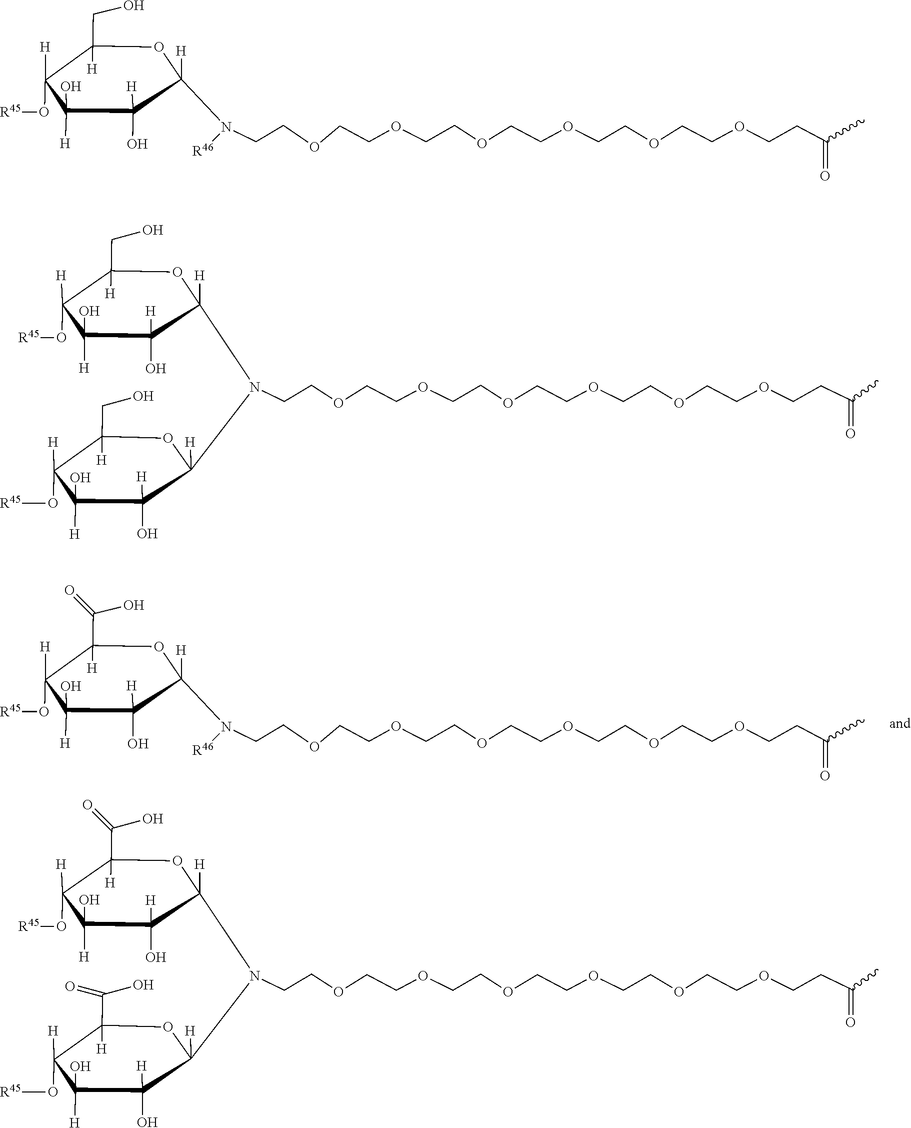
wherein each R⁴⁵ is selected from H and a monosaccharide, a disaccharide, or a polysaccharide; and R⁴⁶ is selected from a cyclic monosaccharide, disaccharide, or polysaccharide; and the wavy line is an attachment to the Amino Acid unit.

22. The Linker compound of any one of claims 1-2 or 4-11, wherein R⁴ and R⁵ are independently selected from a linear monosaccharide and a substituted linear monosaccharide, wherein the substituted linear monosaccharide is substituted with a monosaccharide, a disaccharide or a polysaccharide.

23. The Linker compound of claim 22, wherein —(NR⁴R⁵) is selected from the following, or a stereoisomer or salt thereof: 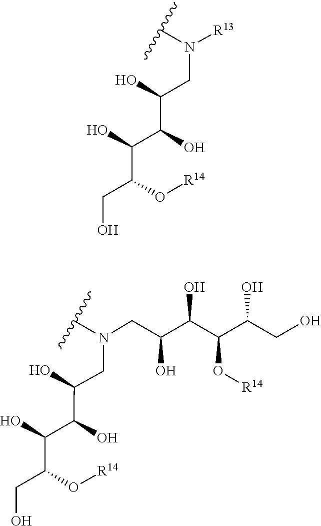
wherein R¹³ is a linear monosaccharide; and each R¹⁴ is selected from a monosaccharide, a disaccharide and a polysaccharide.

24. The Linker compound of claim 22, comprising a Polar group selected from the following, or a stereoisomer or salt thereof: 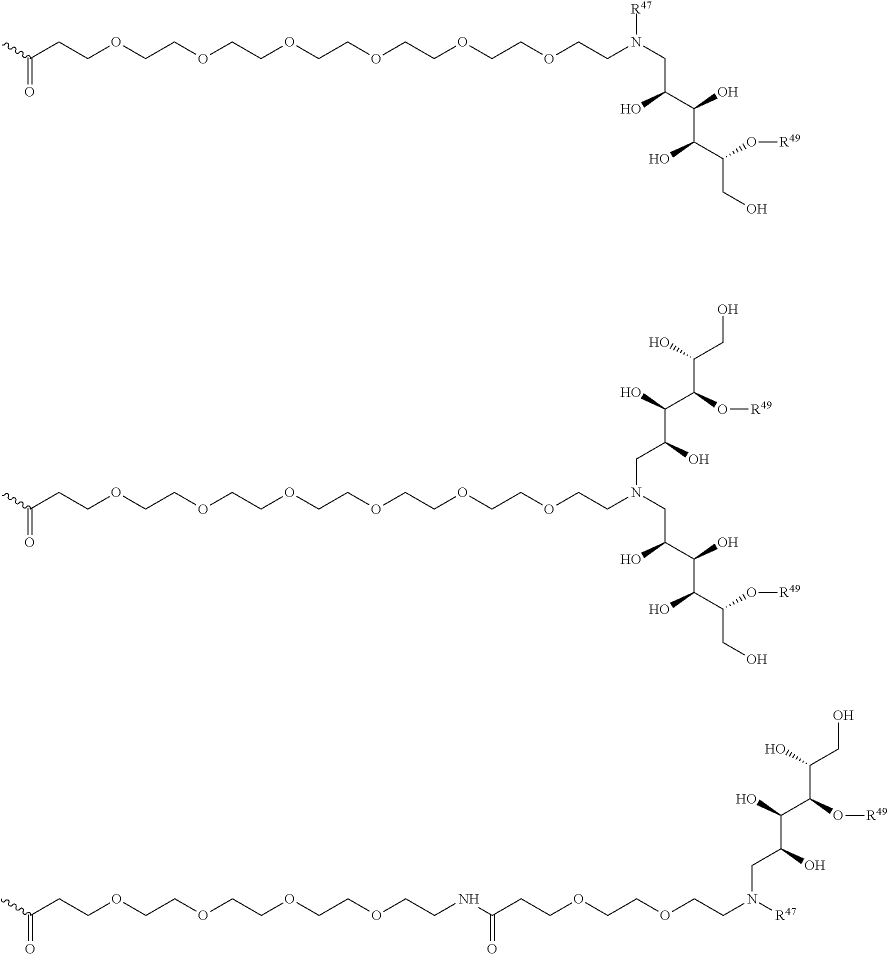 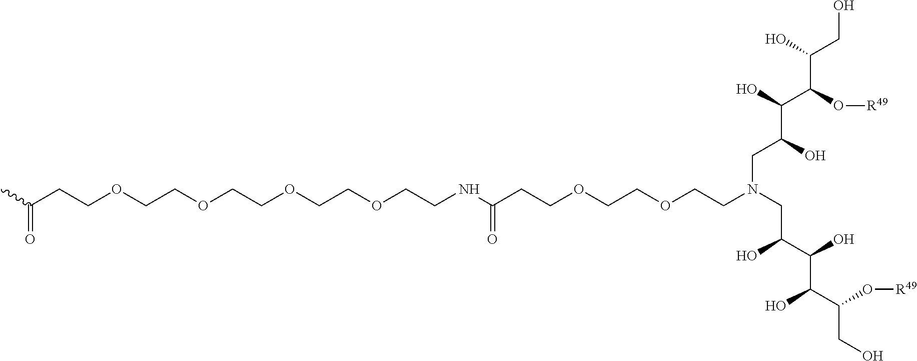
wherein R⁴⁷ is a linear monosaccharide; and each R⁴⁹ is selected from a monosaccharide, a disaccharide and a polysaccharide; and the wavy line is an attachment to the Amino Acid unit.

25. The Linker compound of any one of claims 1-2 or 4-11, wherein R⁴ and R⁵ are independently selected from a linear monosaccharide and a substituted monosaccharide, wherein the substituted linear monosaccharide is substituted with one or more substituents selected from carboxyl, ester, and amide, and optionally further substituted with a monosaccharide, disaccharide or a polysaccharide.

26. The Linker compound of claim 25, wherein —(NR⁴R⁵) is selected from the following, or a stereoisomer or salt thereof: 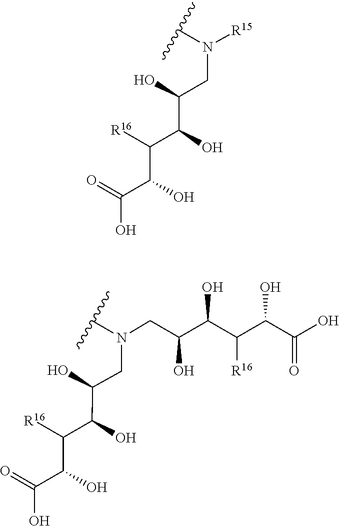
wherein each R¹⁵ is independently selected from a linear monosaccharide and a substituted linear monosaccharide; each R¹⁶ is independently selected from hydroxyl, carboxyl, ester, and amide.

27. The Linker compound of claim 25, comprising a Polar group selected from the following, or a stereoisomer or salt thereof: 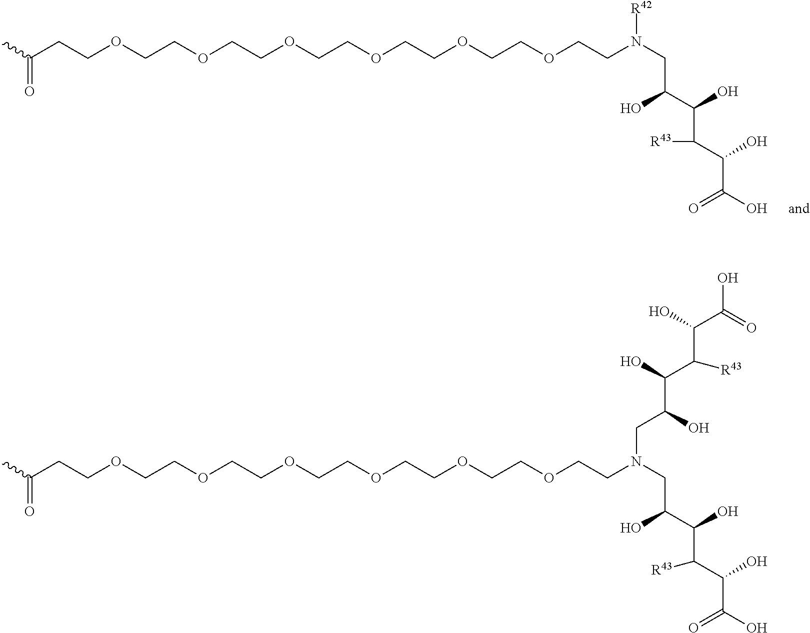
wherein each R⁴² is independently selected from a linear monosaccharide and a substituted linear monosaccharide; each R⁴³ is independently selected from hydroxyl, carboxyl, ester, and amide; and the wavy line is an attachment to the Amino Acid unit.

28. The Linker compound of any one of claims 1-2 or 4-11, wherein one of R⁴ and R⁵ is a —C(O)-polyhydroxyl group or substituted —C(O)-polyhydroxyl group, and the other of R⁴ and R⁵ is a H, —C(O)-polyhydroxyl group, substituted —C(O)-polyhydroxyl group, polyhydroxyl group or substituted polyhydroxyl group; wherein the substituted —C(O)-polyhydroxyl group and polyhydroxyl group are substituted with a monosaccharide, a disaccharide, a polysaccharide, carboxyl, ester, or amide.

29. The Linker compound of claim 28, wherein —(NR⁴R⁵) is selected from the following, or a stereoisomer or salt thereof:

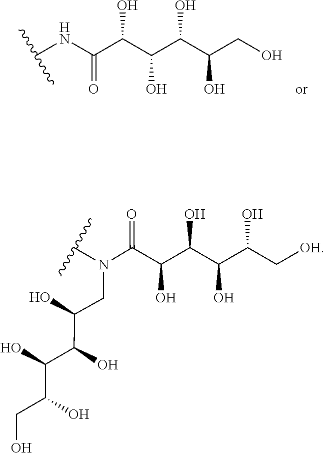 [SMILES: *NC(=O)[C@H](O)[C@@H](O)[C@H](O)[C@H](O)CO ; "or" ; *N(C[C@H](O)[C@@H](O)[C@H](O)[C@H](O)CO)C(=O)[C@H](O)[C@@H](O)[C@H](O)[C@H](O)CO]

30. The Linker compound of claim 28, comprising a Polar group selected from the following, or a stereoisomer or salt thereof: 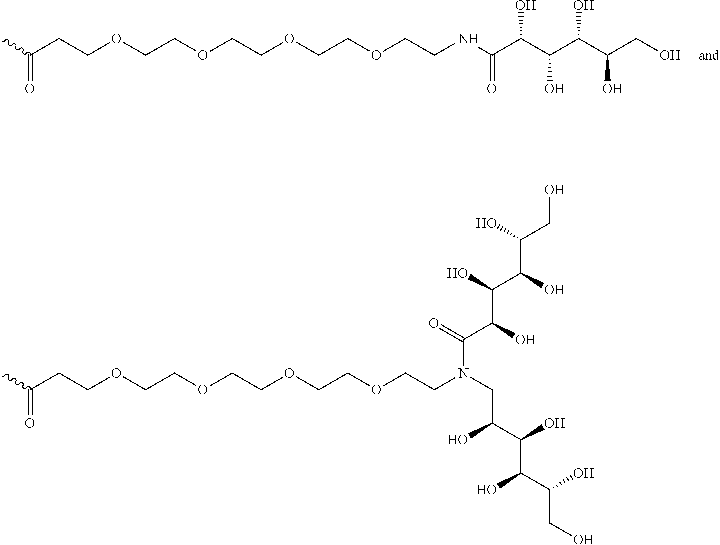
wherein the wavy line is an attachment to the Amino Acid unit.

31. The Linker compound of any one of claims 1-2 or 4-11, wherein —(NR⁴R⁵) is selected from the following, or a stereoisomer or salt thereof: 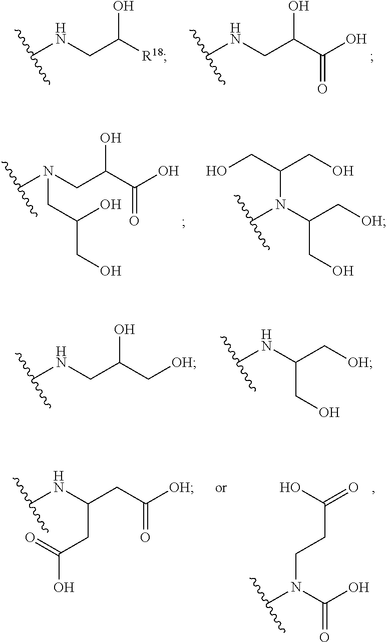
wherein R¹⁸ is selected from OH, CH₂OH, COOH or —C₁-C₆ alkyl substituted with hydroxyl or carboxyl.

32. The Linker compound of any one of claims 1-2 or 4-11, comprising a Polar group selected from the following, or a stereoisomer or salt thereof: 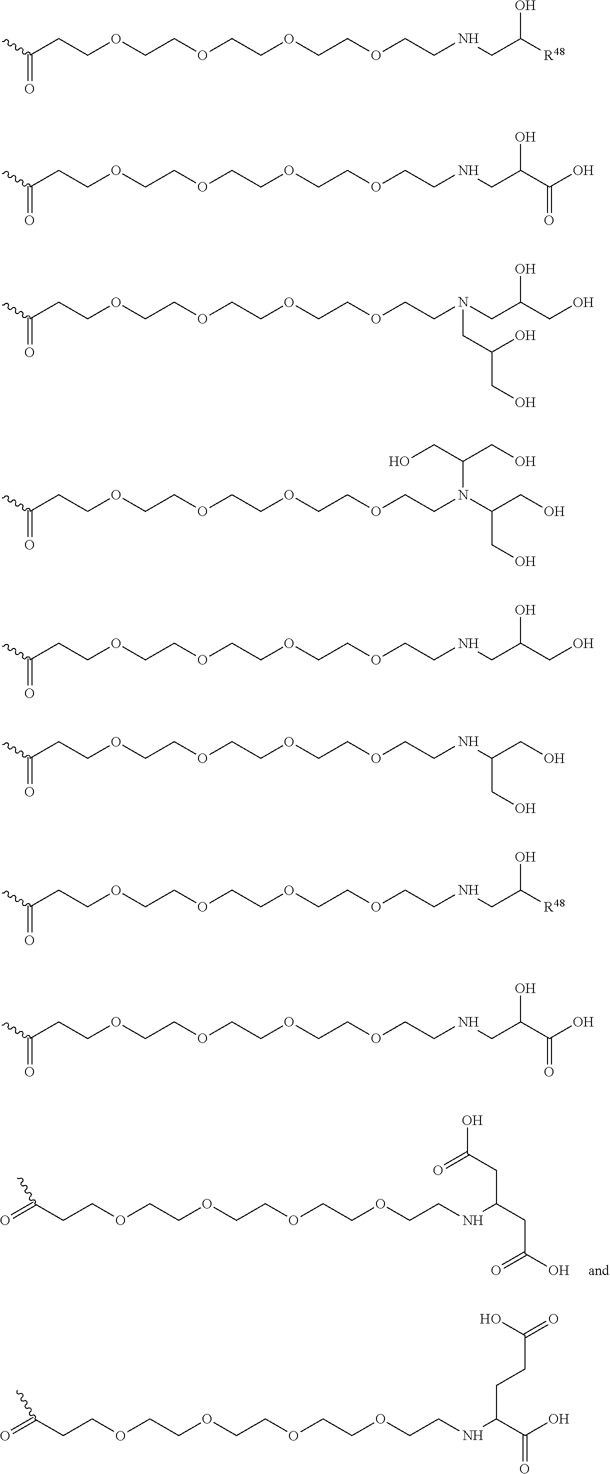
wherein R⁴⁸ is selected from OH, CH₂OH, COOH or —C₁-C₆ alkyl substituted with hydroxyl or carboxyl; and the wavy line is an attachment to the Amino Acid unit.

33. The Linker compound of any one of claims 1-2 or 4-11, wherein —(NR⁴R⁵) is selected from the following, or a stereoisomer or salt thereof:

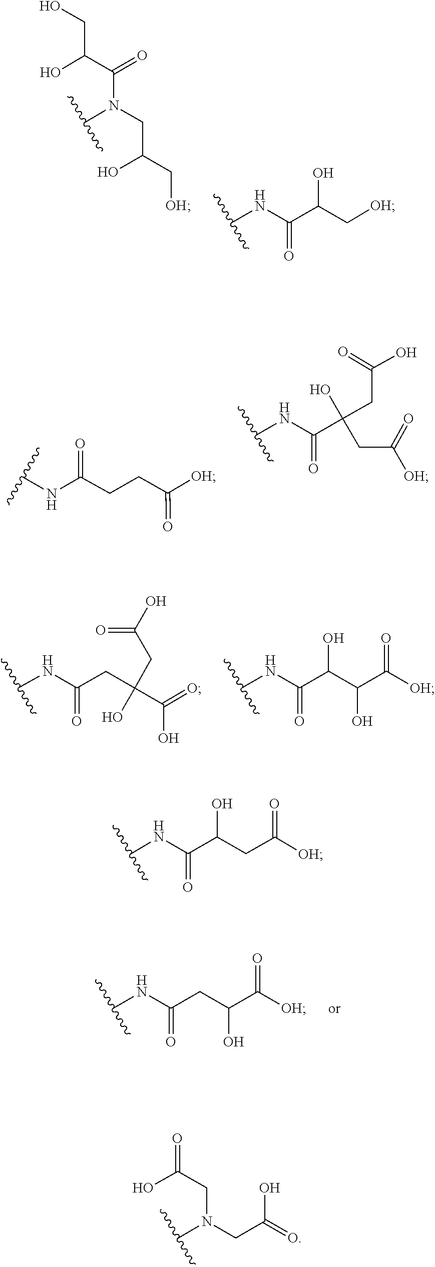

34. The Linker compound of any one of claims 1-2 or 4-11, comprising a Polar group selected from the following, or a stereoisomer or salt thereof: 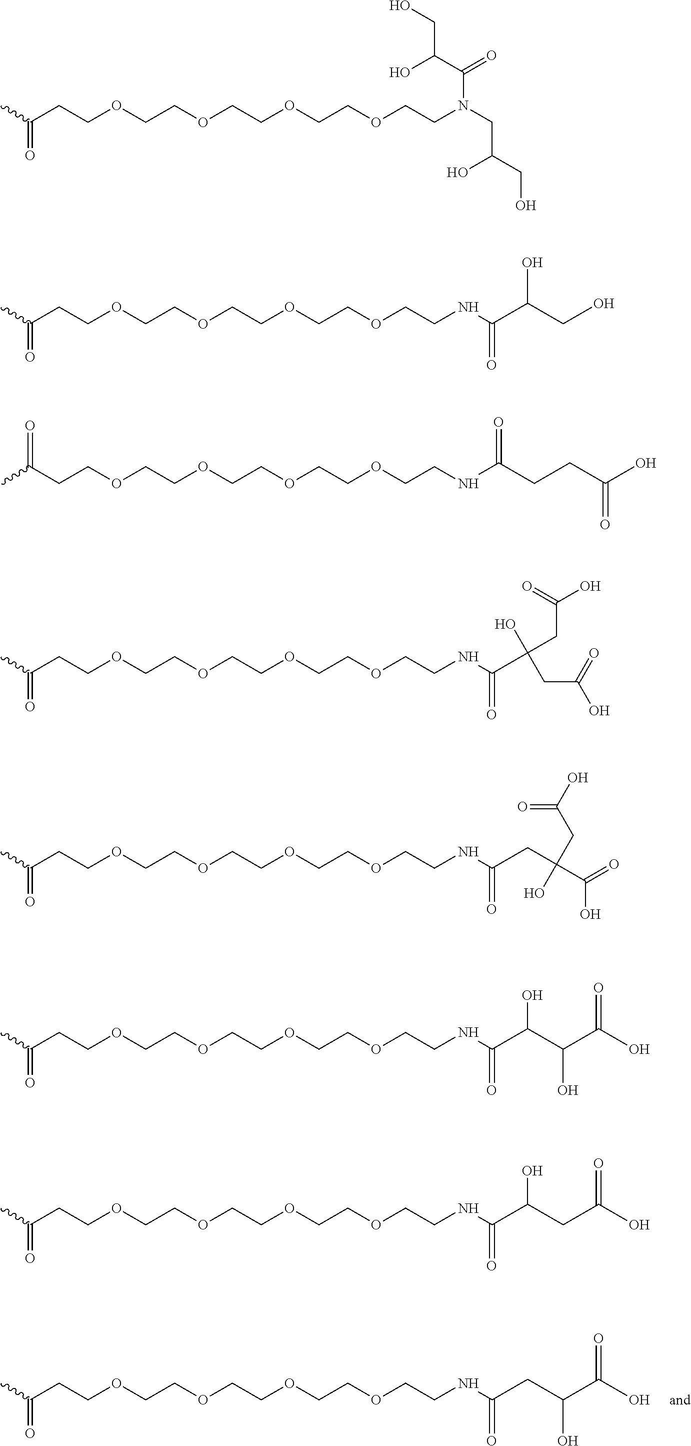 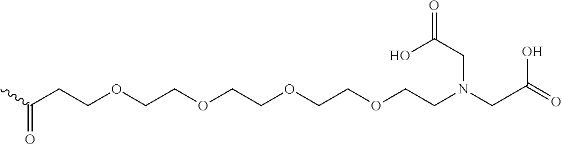
wherein the wavy line is an attachment to the Amino Acid unit.

35. The Linker compound of any one of claims 1-2 or 4-11, wherein R⁴ and R⁵ are independently selected from H and a chelator, provided that both R⁴ and R⁵ are not H.

36. The Linker compound of claim 35, wherein the chelator is optionally attached to the nitrogen of —NR⁴R⁵ by an alkylene, arylene, carbocyclyl, heteroarylene or heterocarbocyclyl.

37. The Linker compound of claim 37, wherein the chelator is selected from ethylenediaminetetraacetic acid (EDTA), diethylenetriaminepentaacetic acid (DTPA), triethylenetetraminehexaacetic acid (TTHA), benzyl-DTPA, 1,4,7,10-tetraazacyclododecane-N,N′,N″,N′″-tetraacetic acid (DOTA), benzyl-DOTA, 1,4,7-triazacyclononane-N,N′,N″-triacetic acid (NOTA), benzyl-NOTA, 1,4,8,11-tetraazacyclotetradecane-1,4,8,11-tetraacetic acid (TETA) and N,N′-dialkyl substituted piperazine.

38. The Linker compound of claim 37, comprising a Polar group selected from the following: 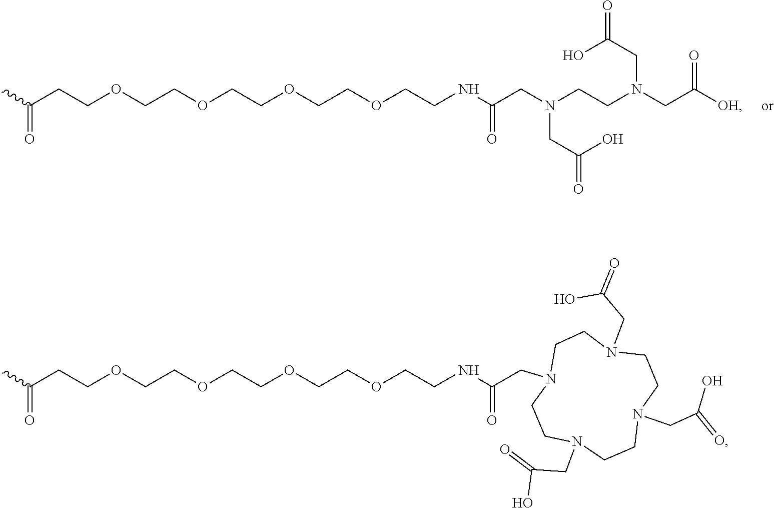
or a stereoisomer or salt thereof wherein the wavy line is an attachment to the Amino Acid unit.

39. The Linker compound of any one of claims 1-2 or 4-11, wherein R⁴ and R⁵ are independently selected from a H, a polyhydroxyl-ether group, a substituted polyhydroxyl-ether group.

40. The Linker compound of claim 39, wherein —(NR⁴R⁵) is selected from the following, or a stereoisomer or salt thereof:

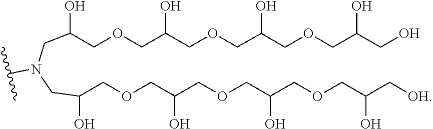 [SMILES: *N(CC(O)COCC(O)COCC(O)COCC(O)CO)CC(O)COCC(O)COCC(O)COCC(O)CO]

41. The Linker compound of any one of claims 1 to 40, comprising a Polar group having a formula selected from the following: 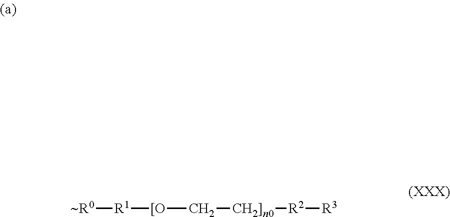
or a stereoisomer or salt thereof, wherein:
R⁰ is a functional group for attachment to a subunit of the Amino Acid unit;
R¹ and R² are each, independently, a bond or C₁-C₃ alkylene groups;
R³ is selected from an optionally substituted C₃-C₁₀ carbocycle, thiourea, optionally substituted thiourea, urea, optionally substituted urea, sulfamide, alkyl sulfamide, acyl sulfamide, optionally substituted alkyl sulfamide, optionally substituted acyl sulfamide, sulfonamide, optionally substituted sulfonamide, guanidine, including alkyl and aryl guanidine, phosphoramide, or optionally substituted phosphoramide; or R³ is selected from azido, alkynyl, substituted alkynyl, —NH—C(O)-alkynyl, —NH—C(O)-alkynyl-R⁵, cyclooctyne; —NH-cyclooctyne, —NH—C(O)-cyclooctyne, or —NH-(cyclooctyne)₂; wherein R⁵ is selected from optionally substituted alkyl, optionally substituted alkenyl, optionally substituted alkynyl, optionally substituted carbocycle, optionally substituted aryl, optionally substituted heterocarbocycle, or optionally substituted heteroaryl; and
n0 is 2 to 26; 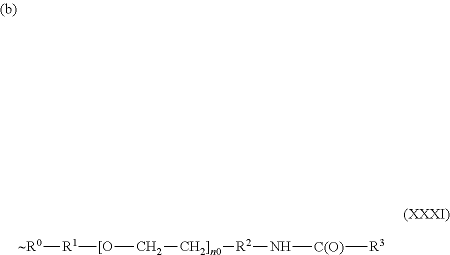
or a stereoisomer or salt thereof, wherein:
R⁰ is a functional group for attachment to a subunit of the Amino Acid unit;
R¹ and R² are each, independently, a bond or C₁-C₃ alkylene groups;
R³ is a branched polyethylene glycol chain, each branch having 1 to 26 ethylene glycol subunits and each branch having an R⁴ at its terminus;
R⁴ is azido, alkynyl, alkynyl-R⁵, cyclooctyne or cyclooctyne-R⁵, wherein R⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n0 is 2 to 26; 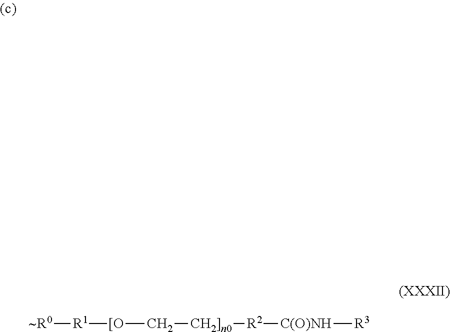
or a stereoisomer or salt thereof, wherein:
R⁰ is a functional group for attachment to a subunit of the Amino Acid unit;
R¹ and R² are each, independently, a bond or C₁-C₃ alkylene groups;
R³ is a branched polyethylene glycol chain, each branch, independently, having 1 to 26 ethylene glycol subunits and each branch having an R⁴ at its terminus;
R⁴ is azido, alkynyl, alkynyl-R⁵, cyclooctyne or cyclooctyne-R⁵, wherein R⁵ is selected from optionally substituted alkyl, optionally substituted alkenyl, optionally substituted alkynyl, optionally substituted carbocycle, optionally substituted aryl, optionally substituted heterocarbocycle and optionally substituted heteroaryl; and
n0 is 2 to 26; 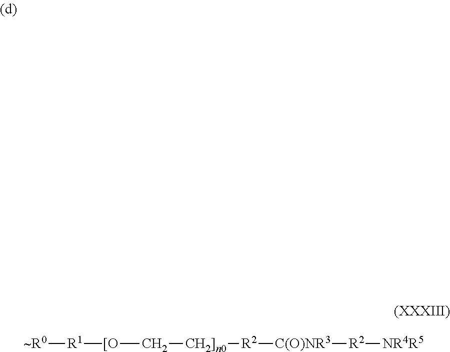
or a stereoisomer or salt thereof, wherein:
R⁰ is a functional group for attachment to a subunit of the Amino Acid unit;
R³ is H or R²—NR⁴R⁵;
R¹ and R² are each, independently, a bond or C₁-C₃ alkylene groups;
R⁴ and R⁵ are each independently selected from a H, polyhydroxyl group, substituted polyhydroxyl group, —C(O)-polyhydroxyl group, substituted —C(O)-polyhydroxyl group, wherein R⁴ and R⁵ are not both H; and
n0 is 2 to 26; 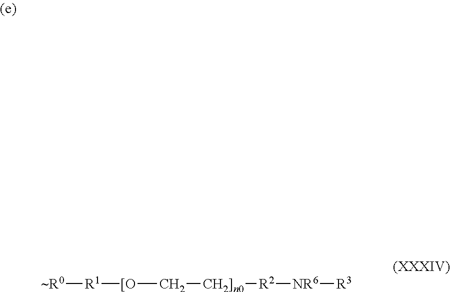
or a stereoisomer or salt thereof, wherein:
R⁰ is a functional group for attachment to a subunit of the Amino Acid unit;
R¹ and R² are each, independently, a bond or C₁-C₃ alkylene groups;
R³ is a branched polyethylene glycol chain, each branch having 1 to 26 ethylene glycol subunits and each branch having an R⁴ at its terminus;
R⁶ is C₁-C₃ alkylene, C₁-C₃ alkylene-C(O), —C(O)—C₁-C₃ alkylene, or —C(O)—C₁-C₃ alkylene-C(O);
R⁴ is azido, alkynyl, alkynyl-R⁵, cyclooctyne or cyclooctyne-R⁵, wherein R⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n0 is 2 to 26; 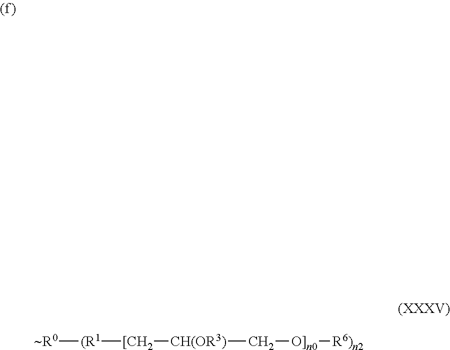
or a stereoisomer or salt thereof, wherein:
R⁰ is a functional group for attachment to a subunit of the Amino Acid unit;
each R¹ is independently a bond, —O— or C₁-C₃ alkylene group;
each R³ is independently H, —[CH₂—CH(OH)—CH₂—O]ₙO—R⁶, —C(O)—NR⁴R⁵ or —C(O)N(R^{N})C₁-C₆alkylene-NR⁴R⁵;
R^{N} is H or C₁-C₄alkyl;
R⁴ and R⁵ are each independently selected from a H, polyhydroxyl group, or substituted polyhydroxyl group, wherein R⁴ and R⁵ are not both H;
each R⁶ is independently H, C₁-C₆alkylene-C(OH)H—NR⁷R⁸, C₁-C₆alkylene-C(OH)H—C₁-C₆alkylene-NR⁷R⁸, —C(O)—NR⁴R⁵, —C(O)N(R^{N})—C₁-C₆alkylene-NR⁴R⁵, C₁-C₆alkylene-C(O)NR⁴R⁵ or C₁-C₆alkylene-CO₂R⁹;
each R⁹ is independently H or C₁-C₆ alkyl;
R⁷ and R⁸ are each independently selected from a H, polyhydroxyl group, substituted polyhydroxyl group, —C(O)-polyhydroxyl group, substituted —C(O)-polyhydroxyl group;
each n0 is independently 1 to 26; and
n2 is 1 or 2; 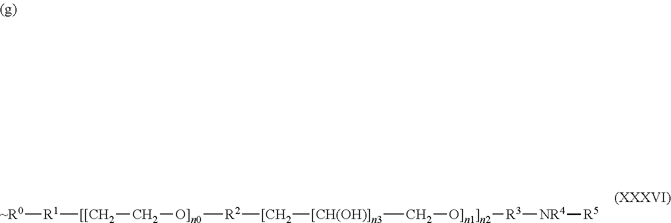
or a stereoisomer or salt thereof, wherein:
R⁰ is a functional group for attachment to a subunit of the Amino Acid unit;
R¹, R² and R³ are each independently a bond or C₁-C₃ alkylene group;
R⁴ and R⁵ are each independently selected from a H, polyhydroxyl group, substituted polyhydroxyl group, —C(O)-polyhydroxyl group, substituted —C(O)-polyhydroxyl group, wherein R⁴ and R⁵ are not both H;
each n0 is independently 0 to 26, and each n1 is independently 0 to 26, with the proviso that at least one of n0 or n1 is 2 to 26;
n2 is Ito 5;
each n3 is independently 1 or 2; 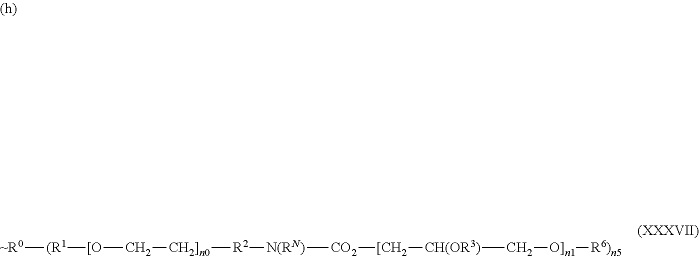
or a stereoisomer or salt thereof, wherein:
R⁰ is a functional group for attachment to a subunit of the Amino Acid unit;
R¹ and R² are each, independently, a bond or C₁-C₃ alkylene groups;
R^{N} is H or C₁-C₄alkyl;
R⁴ and R⁵ are each independently selected from a H, polyhydroxyl group, or substituted polyhydroxyl group, wherein R⁴ and R⁵ are not both H;
each R³ is independently H, —[CH₂—CH(OH)—CH₂—O]ₙ₀—R⁶ or —C(O)N(R^{N})—C₁-C₆alkylene-NR⁴R⁵;
each R⁶ is independently H, C₁-C₆alkylene-C(OH)H—NR⁷R⁸, C₁-C₆alkylene-C(OH)H—C₁-C₆alkylene-NR⁷R⁸, —C(O)N(R^{N})—C₁-C₆alkylene-NR⁴R⁵, C₁-C₆alkylene-C(O)NR⁴R⁵ or C₁-C₆alkylene-CO₂R⁹;
each R⁹ is independently H or C₁-C₆ alkyl;
R⁷ and R⁸ are each independently selected from a H, polyhydroxyl group, substituted polyhydroxyl group, —C(O)-polyhydroxyl group, substituted —C(O)-polyhydroxyl group;
n0 is 2 to 26;
n1 is 1 to 26; and
n5 is 1 or 2; 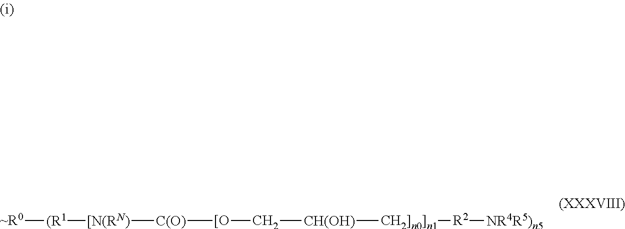
or a stereoisomer or salt thereof, wherein:
R⁰ is a functional group for attachment to a subunit of the Amino Acid unit;
R¹ and R² are each, independently, a bond or C₁-C₃ alkylene groups;
R^{N} is H or C₁-C₄alkyl;
R⁴ and R⁵ are each independently selected from a H, polyhydroxyl group, or substituted polyhydroxyl group, wherein R⁴ and R⁵ are not both H;
n0 is 2 to 26;
n1 is 2 to 4; and
n5 is 1, 2 or 3; 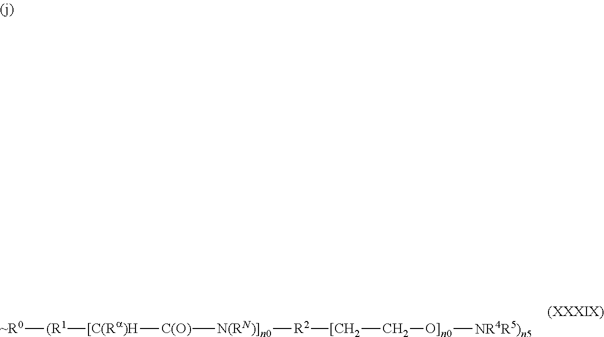
or a stereoisomer or salt thereof, wherein:
R⁰ is a functional group for attachment to a subunit of the Amino Acid unit;
R¹ and R² are each, independently, a bond, C₁-C₃ alkylene, —C₁-C₃alkylene-[O—CH₂—CH₂—]ₙ₀, —[CH₂—CH₂—O]ₙ₀—C₁-C₃alkylene-, or —C₁-C₃alkylene-[O—CH₂—CH₂—]ₙ₀—C(O)—;
each R^{α} is independently H or —R²—NR⁴R⁵;
each R^{N} is independently H, C₁-C₆ alkyl or —R²—NR⁴R⁵;
R⁴ and R⁵ are each independently selected from a H, polyhydroxyl group, substituted polyhydroxyl group, —C(O)-polyhydroxyl group, substituted —C(O)-polyhydroxyl group, a chelator, and —C(O)—R, wherein R is a Sugar unit of formula (XII) or (XIII), wherein R⁴ and R⁵ are not both H;
each n0 is independently 0 to 26, with the proviso that at least one n0 is 2 to 26; and
n5 is 1 or 2; or 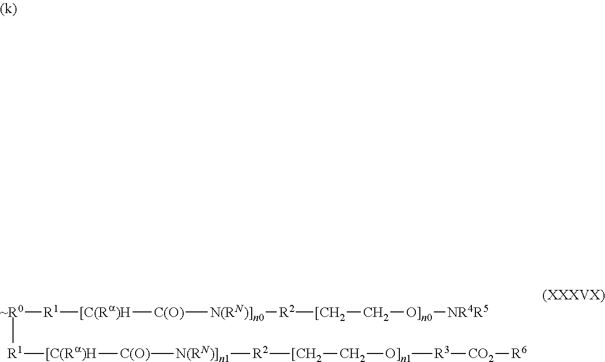
or a stereoisomer or salt thereof, wherein:
R⁰ is a functional group for attachment to a subunit of the Amino Acid unit;
R¹, R² and R³ are each, independently, a bond, C₁-C₃ alkylene, —C₁-C₃alkylene-[O—CH₂—CH₂—]ₙ₀, —[CH₂—CH₂—O]ₙ₀—C₁-C₃alkylene- or —C₁-C₃alkylene-[O—CH₂—CH₂—]ₙ₀—C(O)—;
each R^{α} is independently H or —R²—NR⁴R⁵;
each R^{N} is independently H, C₁-C₆ alkyl or —R²—NR⁴R⁵;
R⁴ and R⁵ are each independently selected from a H, polyhydroxyl group, substituted polyhydroxyl group, —C(O)-polyhydroxyl group, substituted —C(O)-polyhydroxyl group, a chelator, or —C(O)—R, where R is a Sugar unit of formula (XII) or (XIII), wherein R⁴ and R⁵ are not both H;
R⁶ is H or C₁-C₆ alkyl;
each n0 is independently 0 to 26, with the proviso that at least one n0 is 2 to 26; and
each n1 is independently 0 to 26, with the proviso that at least one n1 is 2 to 26.

42. The Linker compound of any one of claims 1-41, comprising a Polar group having a formula selected from the following, or a stereoisomer or salt thereof: 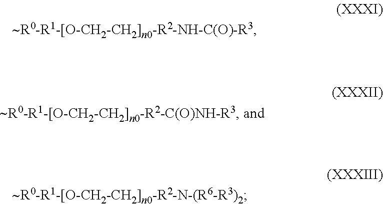
wherein:
R⁰ is a functional group for attachment to a subunit of the Amino Acid unit;
R¹ and R² are each, independently, a bond or C₁-C₃ alkylene groups;
R³ is a branched polyethylene glycol chain, each branch having 1 to 26 ethylene glycol subunits and each branch having an R⁴ at its terminus;
R⁶ is C₁-C₃ alkylene, —C₁-C₃ alkylene-C(O), —C(O)—C₁-C₃ alkylene or —C(O)—C₁-C₃ alkylene-C(O);
R⁴ is azido, alkynyl, alkynyl-R⁵, cyclooctyne or cyclooctyne-R⁵, wherein R⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl;
the wavy line (˜) indicates the attachment site of the Amino Acid unit to R⁰; and
n0 is 2 to 26.

43. The Linker compound of claim 41 or 42, comprising a Polar group formed from a precursor group selected from the following: 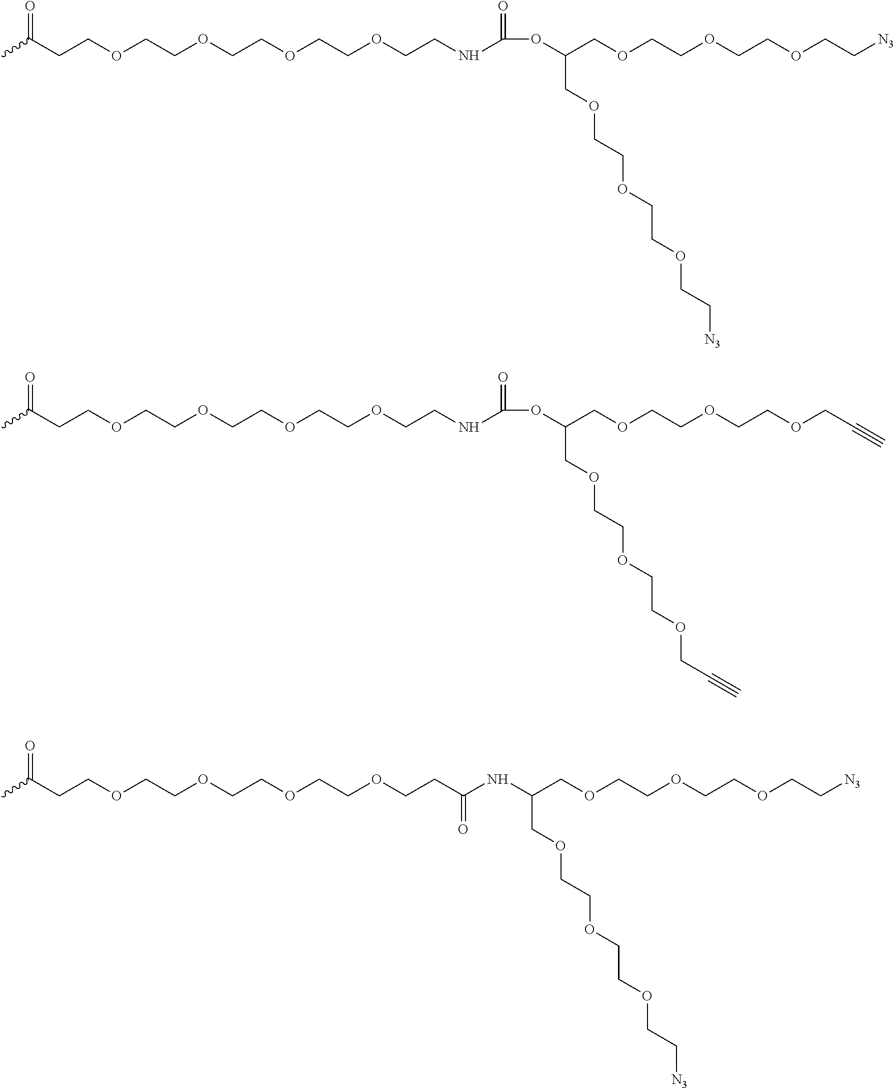 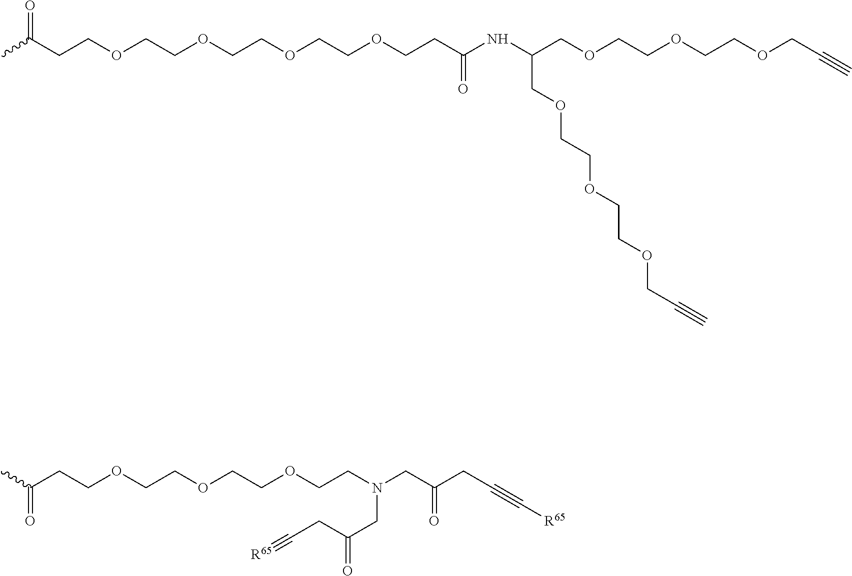
wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and the wavy line is an attachment to the Amino Acid unit.

44. The Linker compound of any one of claims 1-43, comprising a Polar group having a formula: 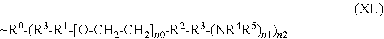
or a stereoisomer or salt thereof, wherein:
R⁰ is a functional group for attachment to a subunit of the Amino Acid unit;
R¹ and R² are each, independently, a bond or C₁-C₆ alkylene;
each R³ is independently selected from a bond, C₁-C₁₂ alkylene, —OC₁-C₁₂ alkylene, —C(═O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, and —C(O)NR⁷R⁸, wherein each alkylene is optionally substituted with hydroxyl, SO₃H and/or oxo, R^{a} is H, C₁-C₆ alkyl, a polyhydroxyl group, or a substituted polyhydroxyl group, and one of R⁷ and R⁸ is H or C₁-C₁₂ alkylene and the other is C₁-C₁₂ alkylene, wherein one of the C₁-C₂ alkylenes is bound to NR⁴⁴R⁴⁵ at the nitrogen atom;
R⁴ and R⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, and wherein R⁴ and R⁵ are not both H;
n0 is 2 to 26;
n1 is 1 to 6; and
n2 is 1 to 6.

45. The Linker compound of claims 1-44, comprising a Polar group having a formula: 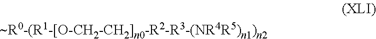
or a stereoisomer or salt thereof, wherein:
R⁰ is a functional group for attachment to a subunit of the Amino Acid unit;
R¹ and R² are each, independently, a bond or C₁-C₆ alkylene;
each R³ is independently selected from a bond, C₁-C₁₂ alkylene, —OC₁-C₁₂ alkylene, —C(═O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, C(O)—NR^{a}—C₁-C₁₂ alkylene, -heteroarylene, heteroaryl-C₁-C₁₂ alkylene, heteroaryl-C₁-C₁₂ alkylene-C(O)—, or —C(O)NR⁷R⁸, wherein each alkylene is optionally substituted with hydroxyl, SO₃H and/or oxo, R^{a} is H, C₁-C₆ alkyl, a polyhydroxyl group, or a substituted polyhydroxyl group and one of R⁷ and R⁸ is H or C₁-C₁₂ alkylene and the other is C₁-C₁₂ alkylene, wherein one of the C₁-C₂ alkylenes is bound to NR⁴⁴R⁴⁵ at the nitrogen atom;
R⁴ and R⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, and wherein R⁴ and R are not both H;
n0 is 2 to 26;
n1 is Ito 6; and
n2 is Ito 6.

46. The Linker compound of claims 1-45, comprising a Polar group having a formula: 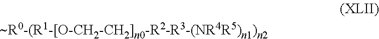
or a stereoisomer or salt thereof, wherein:
R⁰ is a functional group for attachment to a subunit of the Amino Acid unit;
R¹ and R² are each, independently, a bond or C₁-C₃ alkylene;
each R³ is independently selected from a bond, C₁-C₆ alkylene, —OC₁-C₁₂ alkylene, —C(═O)—, —NR^{a}—C₁-C₁₂ alkylene, —C₁-C₆ alkylene-NR^{a}—, —C(O)—C₁-C₆ alkylene, —C₁-C₆ alkylene-C(O)—, —NR^{a}C₁-C₆ alkylene-C(O)—, —C(O)—C₁-C₆ alkylene-NR^{a}—, —NR^{a}—C(O)—NR^{a}—, —NR^{a}—C(O)—, —NR^{a}—C(O)—C₁-C₆ alkylene, —C(O)—NR^{a}—C₁-C₁₂ alkylene, -heteroarylene, heteroaryl-C₁-C₆ alkylene, heteroaryl-C₁-C₆ alkylene-C(O)—, and —C(O)NR⁷R⁸, wherein each alkylene is optionally substituted with hydroxyl, SO₃H, and/or oxo, R^{a} is H, C₁-C₆ alkyl, a polyhydroxyl group, or a substituted polyhydroxyl group and one of R⁷ and R⁸ is H or C₁-C₆ alkylene and the other is C₁-C₁₂ alkylene, wherein one of the C₁-C₂ alkylenes is bound to NR⁴⁴R⁴⁵ at the nitrogen atom;
R⁴ and R⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, and wherein R⁴ and R⁵ are not both H;
n0 is 2 to 16;
n1 is 1 to 4; and
n2 is 1 to 4.

47. The Linker compound of any one of claims 1-2 or 4-46, wherein R⁰ derives from a functional group of a precursor compound to the Polymer unit, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, or protected forms thereof.

48. The Linker compound of any one of claims 1-2 or 4-47, wherein R⁶ has one of the following structures: 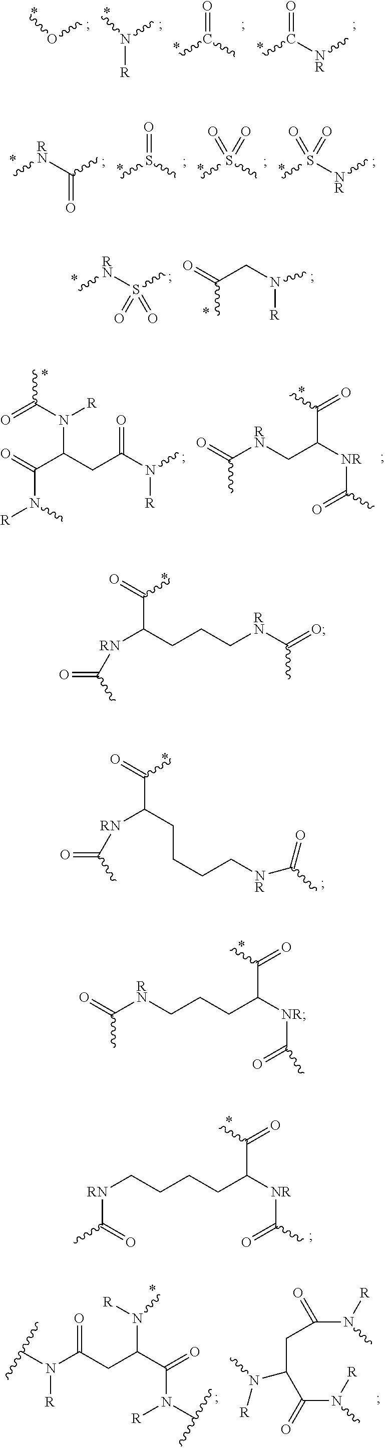 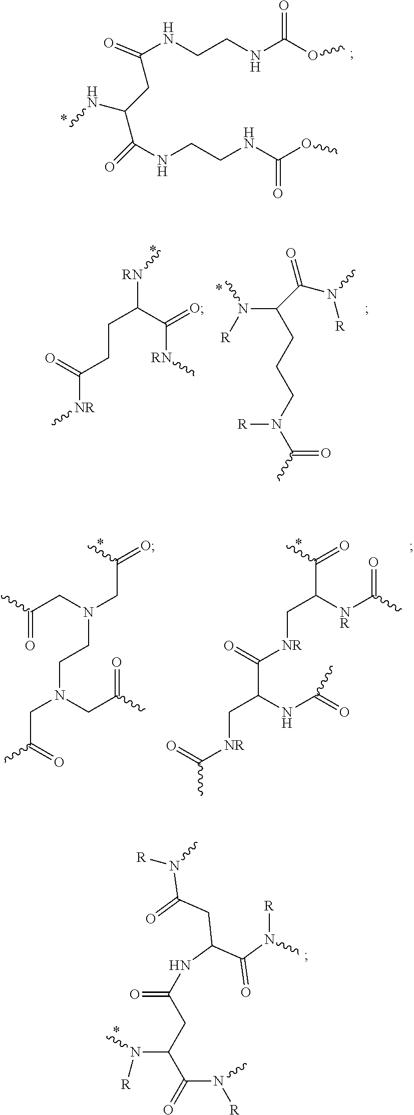 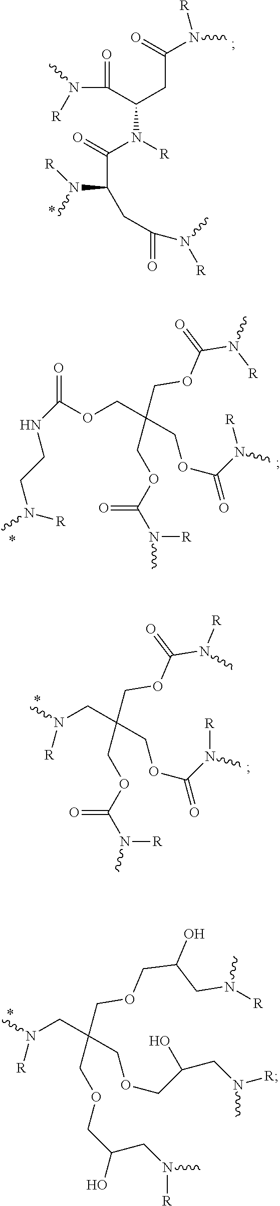 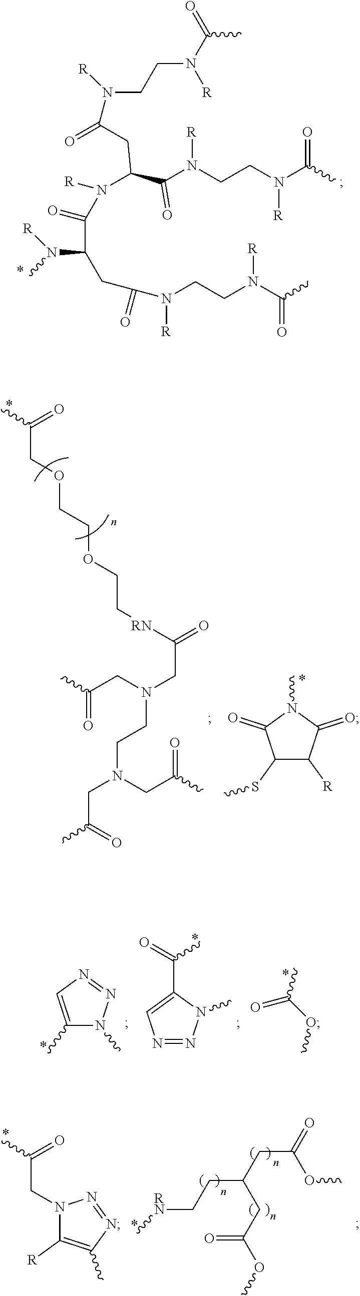 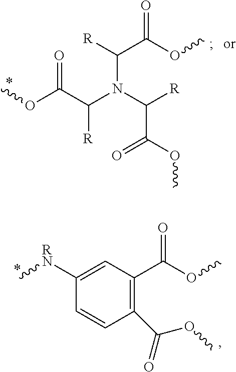
or a stereoisomer thereof, wherein R is H, C₁-C₆ alkyl or polyhydroxyl group, n is 0 to 12, the (*) indicates the attachment site of R⁶ to a subunit of the Amino Acid unit and each ( [symbol] ) indicates the attachment site of R⁰ to the remainder of the Polymer unit.

49. The compound of claim 48, wherein R⁰ has one of the following structures: 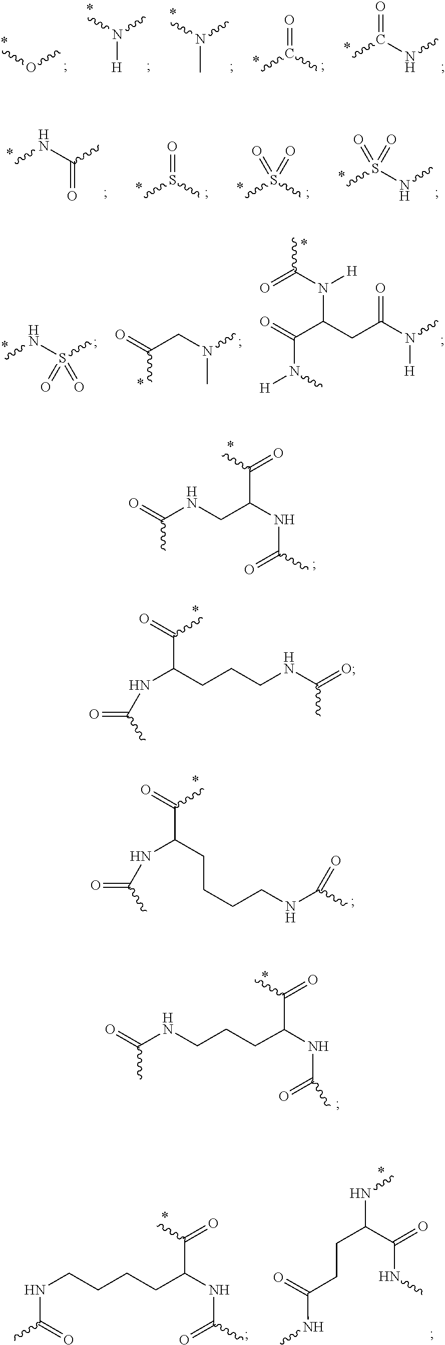 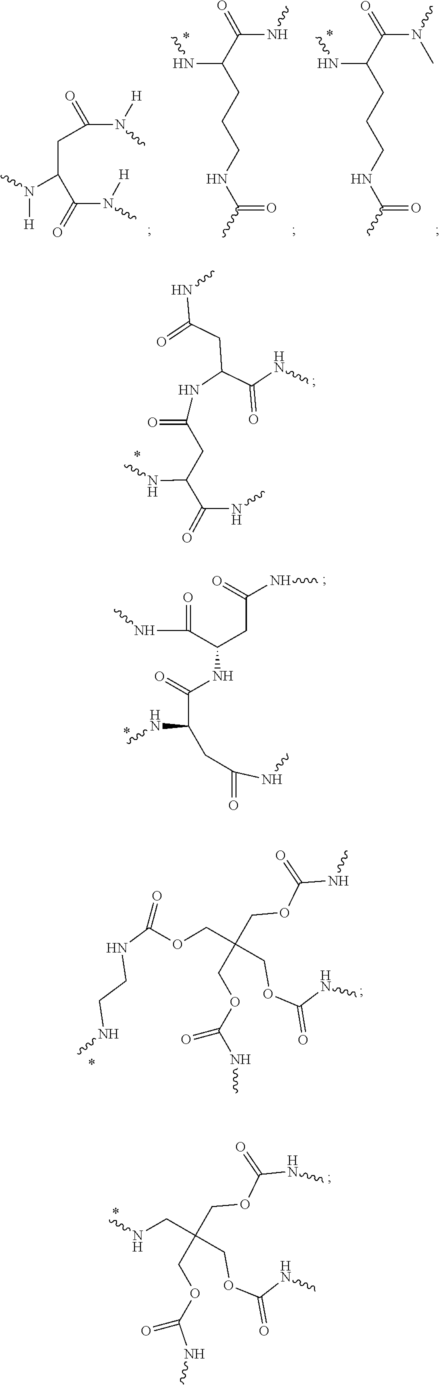 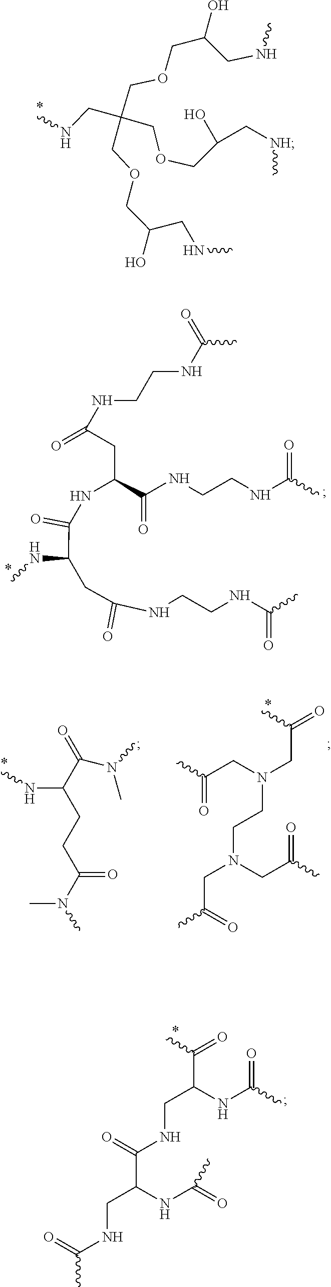 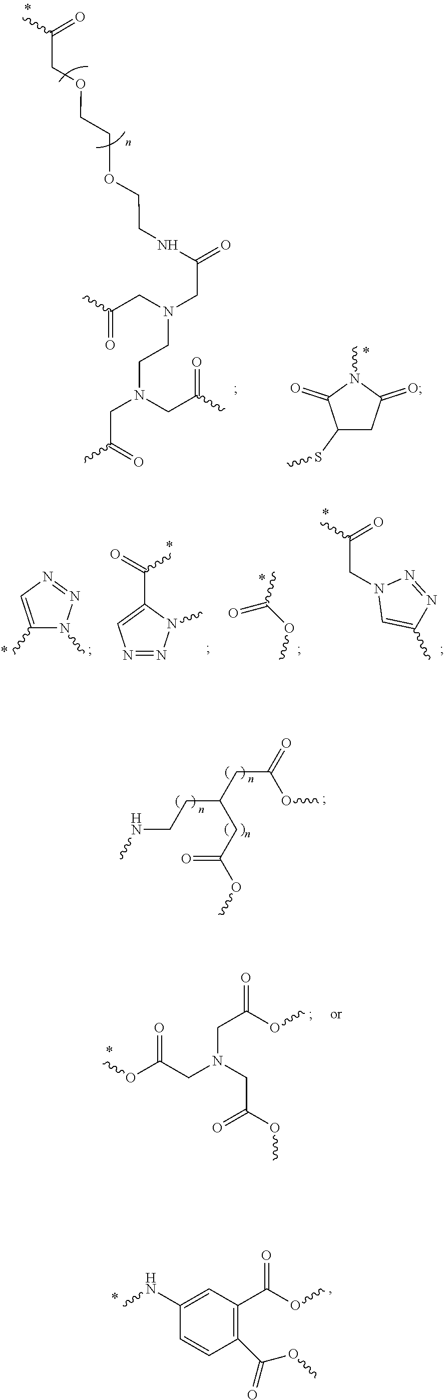
or a stereoisomer thereof, wherein R is H, C₁-C₆ alkyl or polyhydroxyl group, n is 0 to 12, the (*) indicates the attachment site of R⁰ to a subunit of the Amino Acid unit and each ( [symbol] ) indicates an attachment site of R⁰ to the remainder of the Polymer unit.

50. The Linker compound of any one of claims 1-2 or 6-30, wherein —R³—(NR⁴R⁵)ₙ₁, when R³ is present, has one of the following structures: 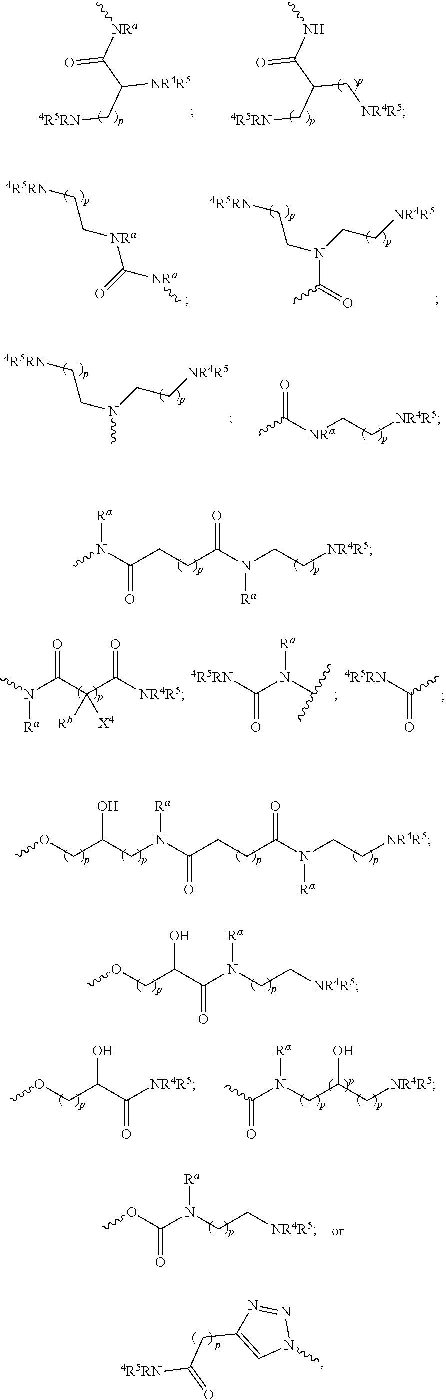
or a stereoisomer thereof, wherein each R^{a} and R^{b} are independently H or C₁₋₆ alkyl, X⁴ is SO₃H, p is 0-8, and the ( [symbol] ) indicates the attachment site of R³ to the remainder of the Polymer unit.

51. The Linker compound of claim 50, wherein —R³—(NR⁴R⁵)ₙ₁, when R³ is present, has one of the following structures: 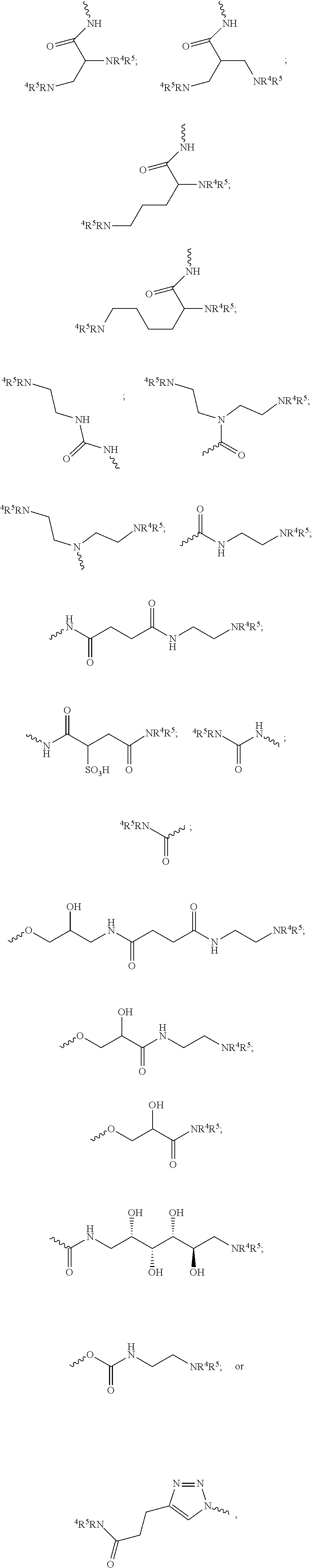
or a stereoisomer thereof, wherein the ( [symbol] ) indicates the attachment site of R³ to the remainder of the Polymer unit.

52. The Linker compound of any one of claims 1-51, wherein at least one —NR⁴R⁵, when present, has one of the following structures: 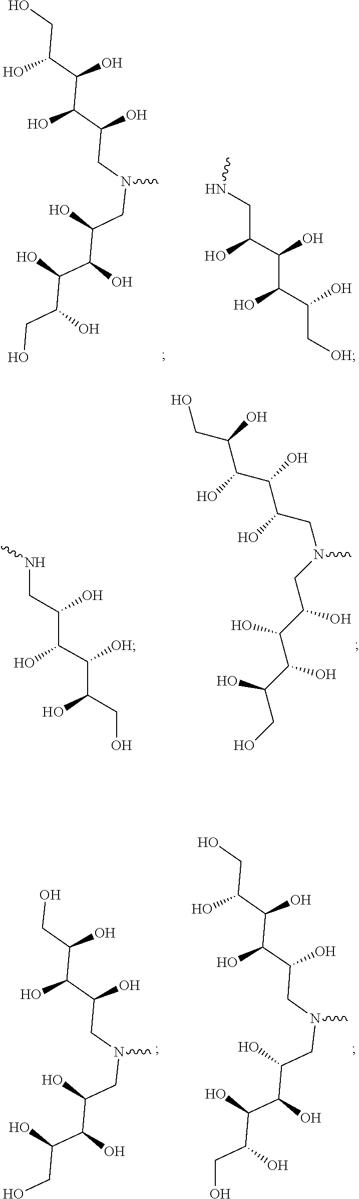 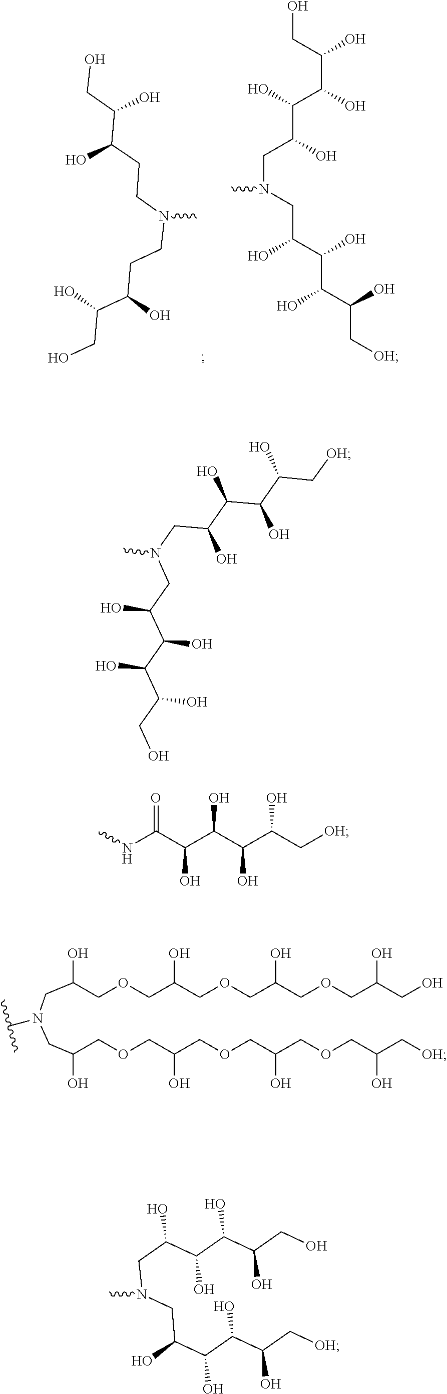 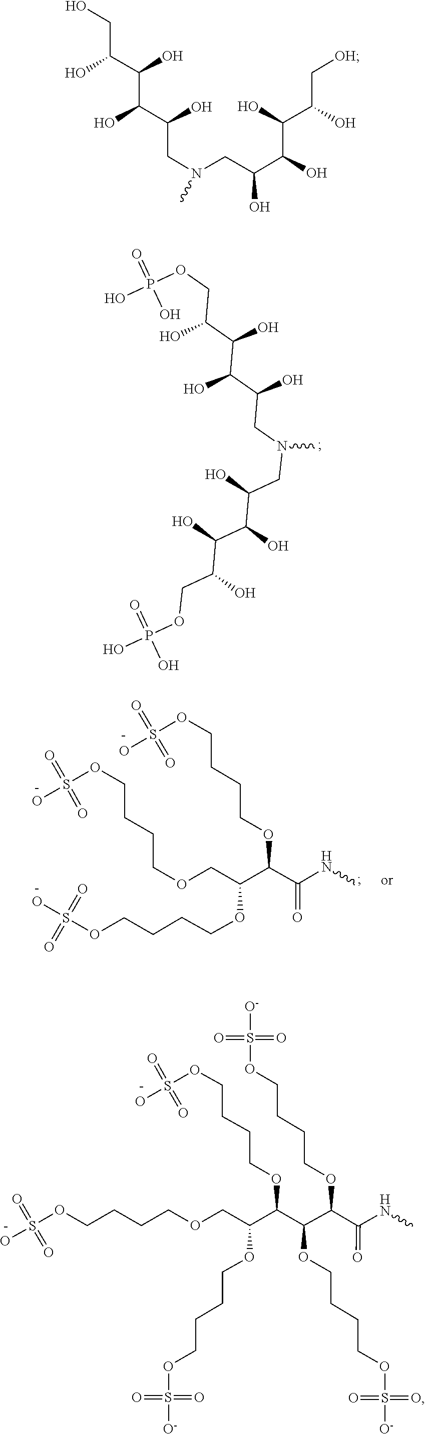
or a stereoisomer thereof, wherein the ( [symbol] ) indicates the attachment site of —NR⁴R⁵ to the remainder of the Polymer unit.

53. The Linker compound of any one of claims 1-52, comprising a Polar group having one of the following structures prior to attachment to the Linker unit: 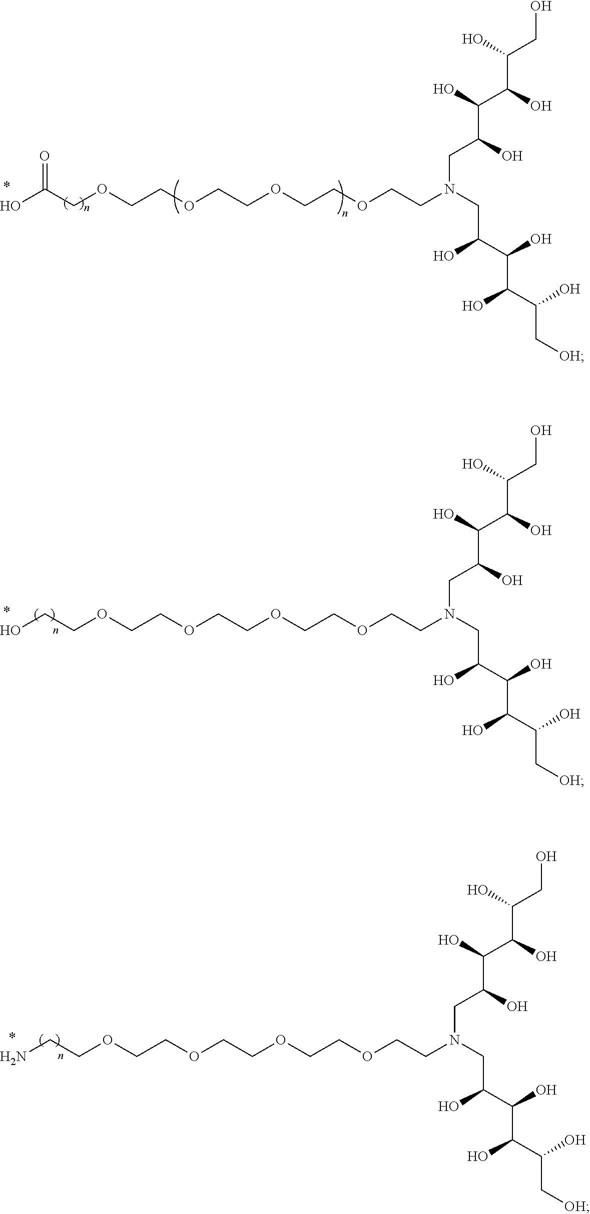 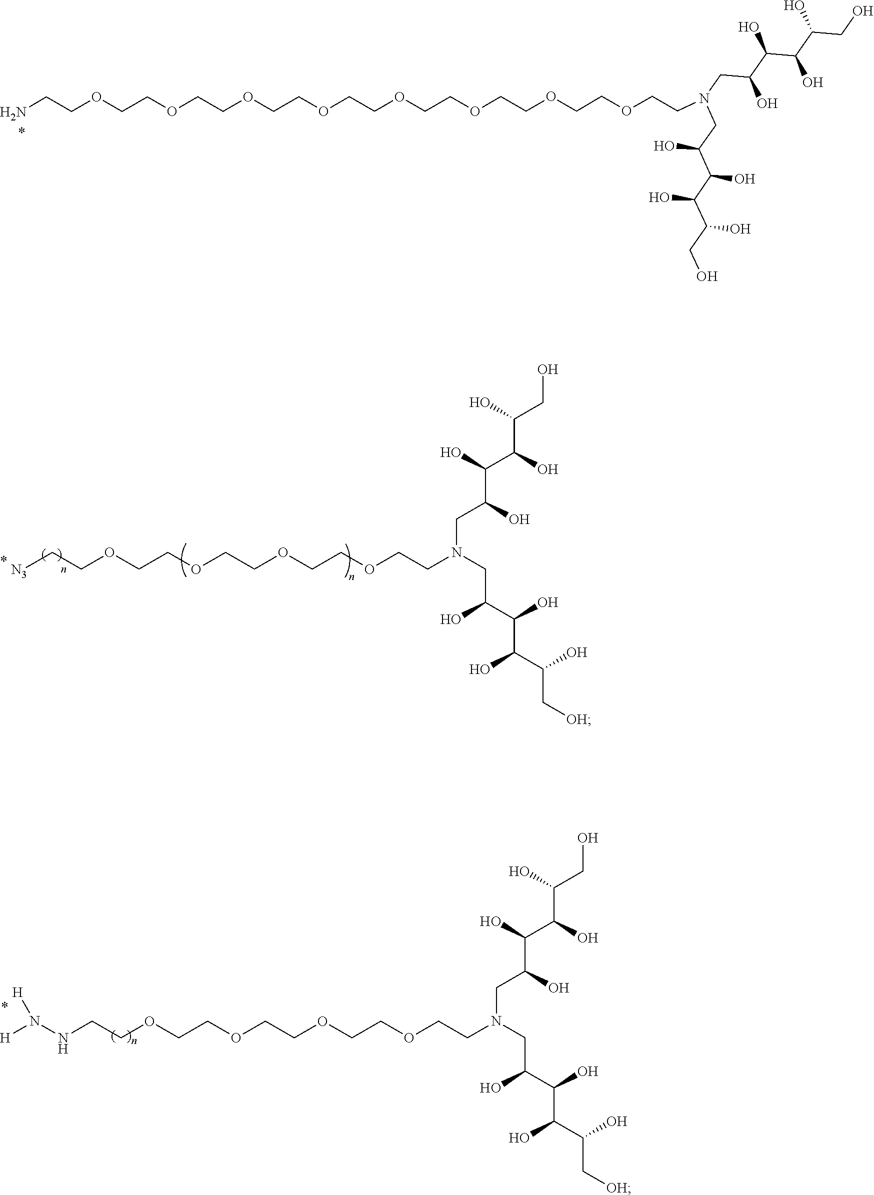 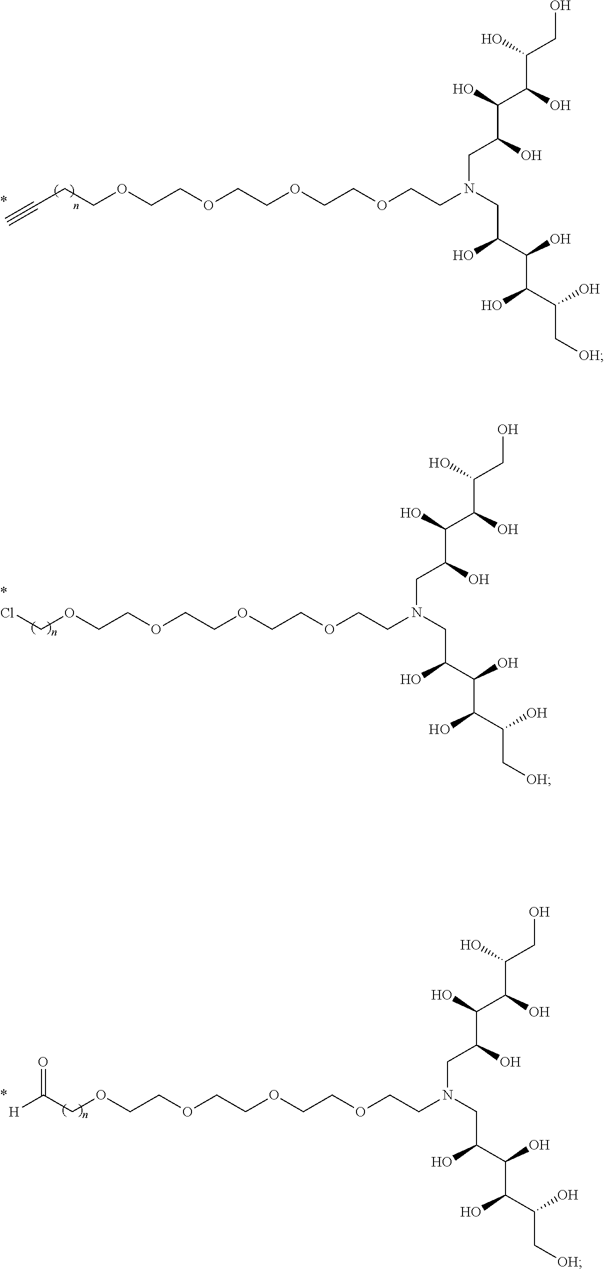 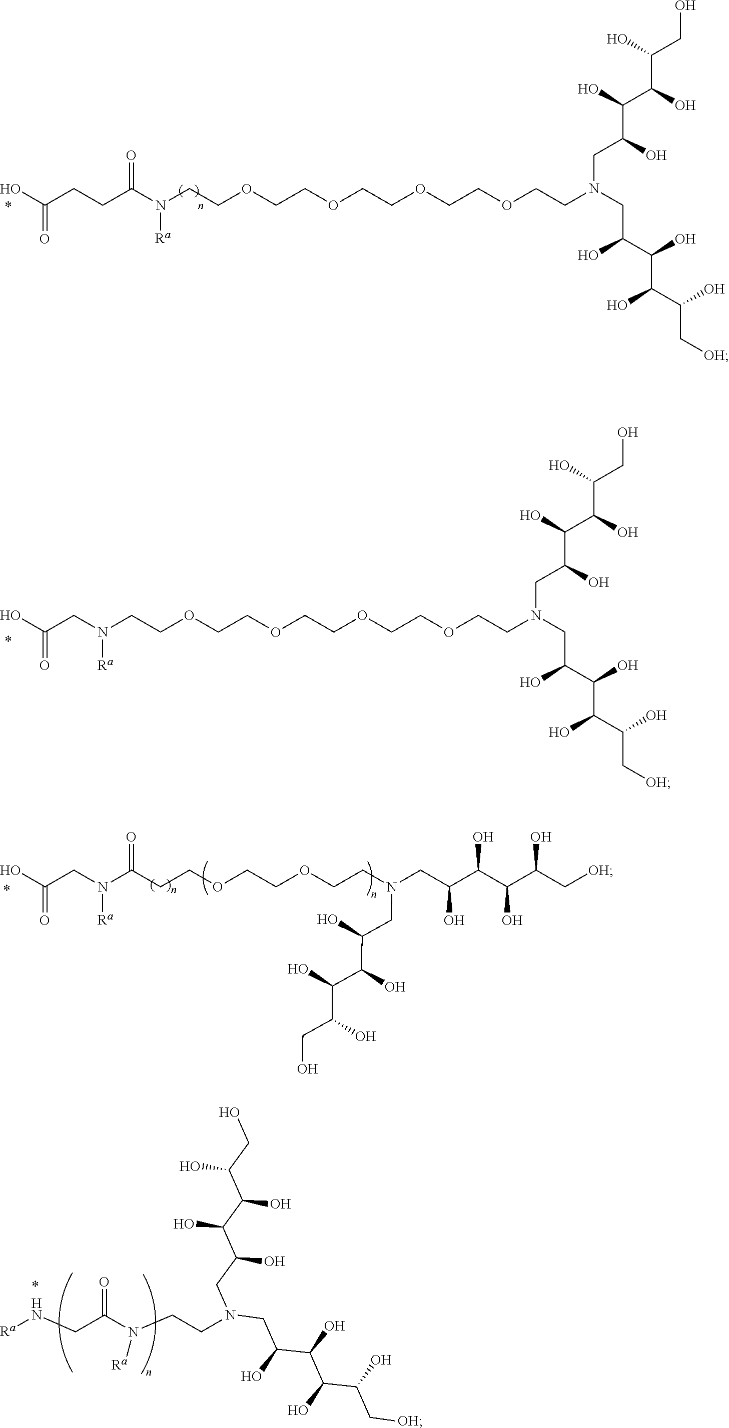 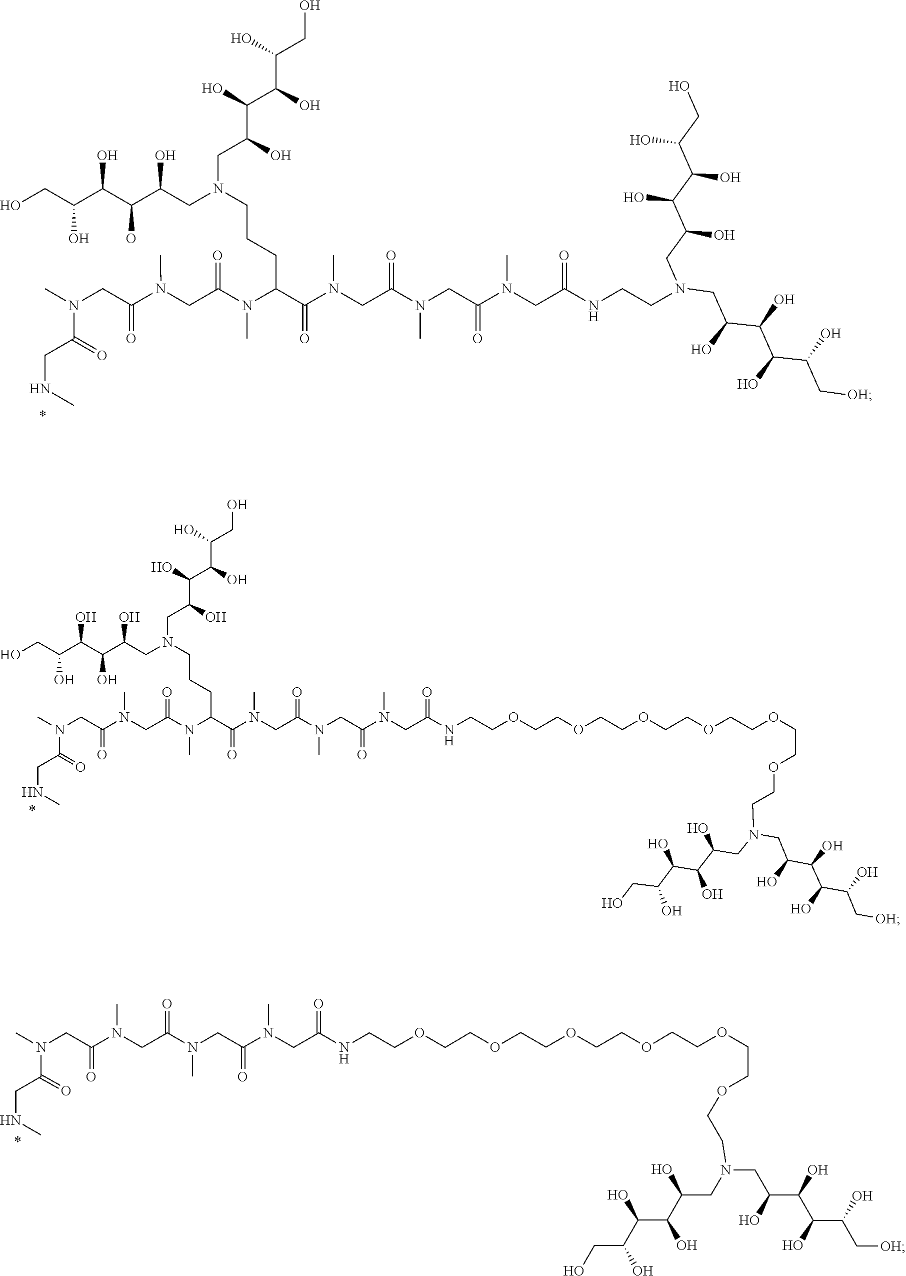 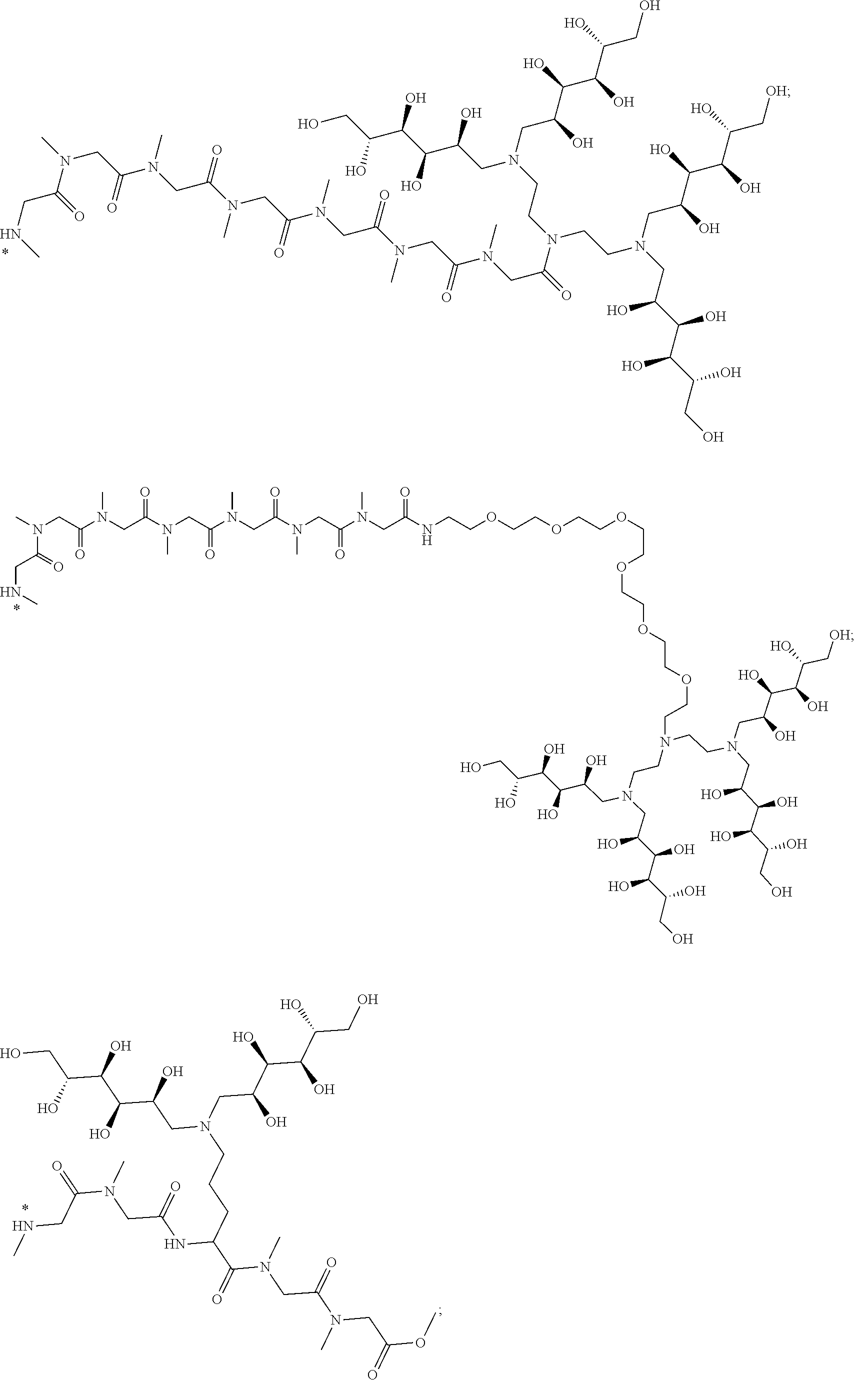 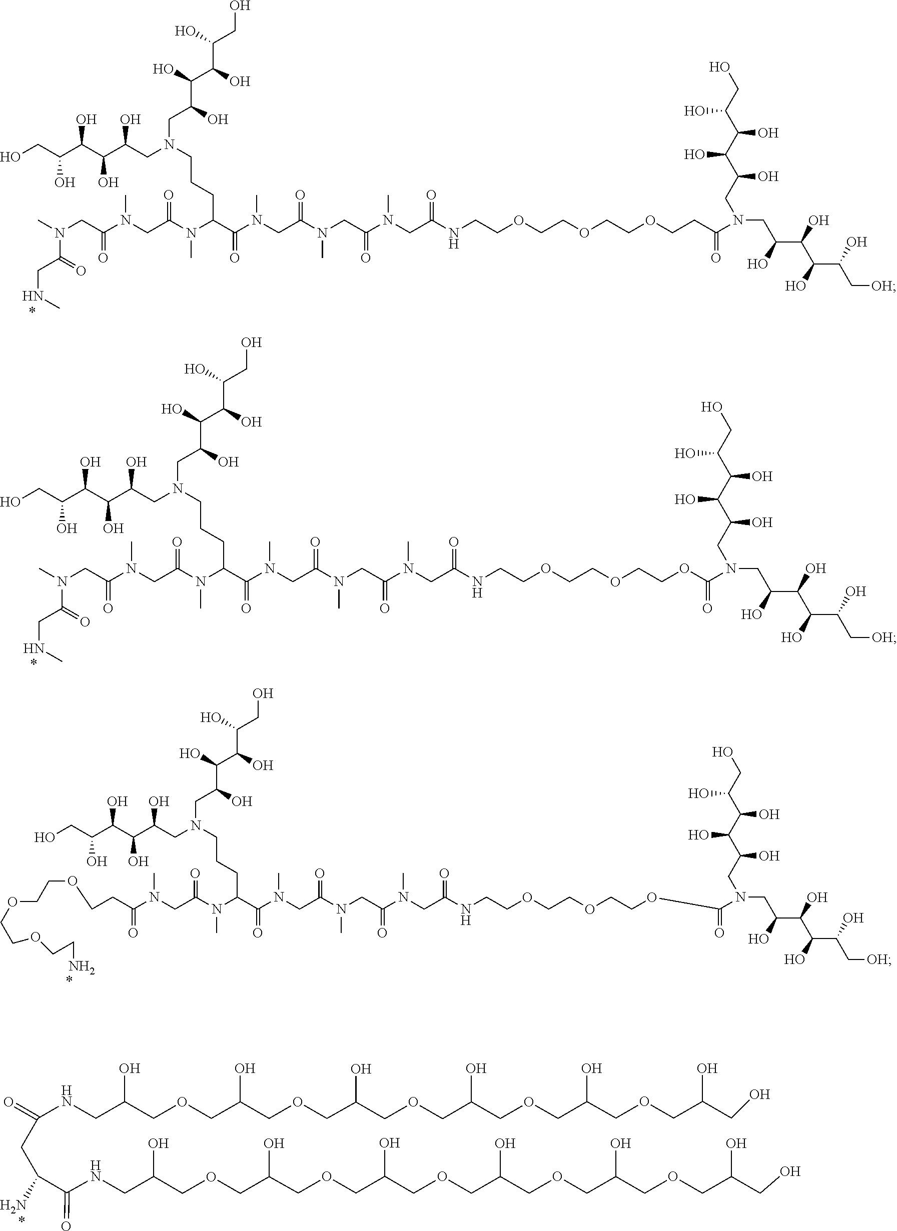 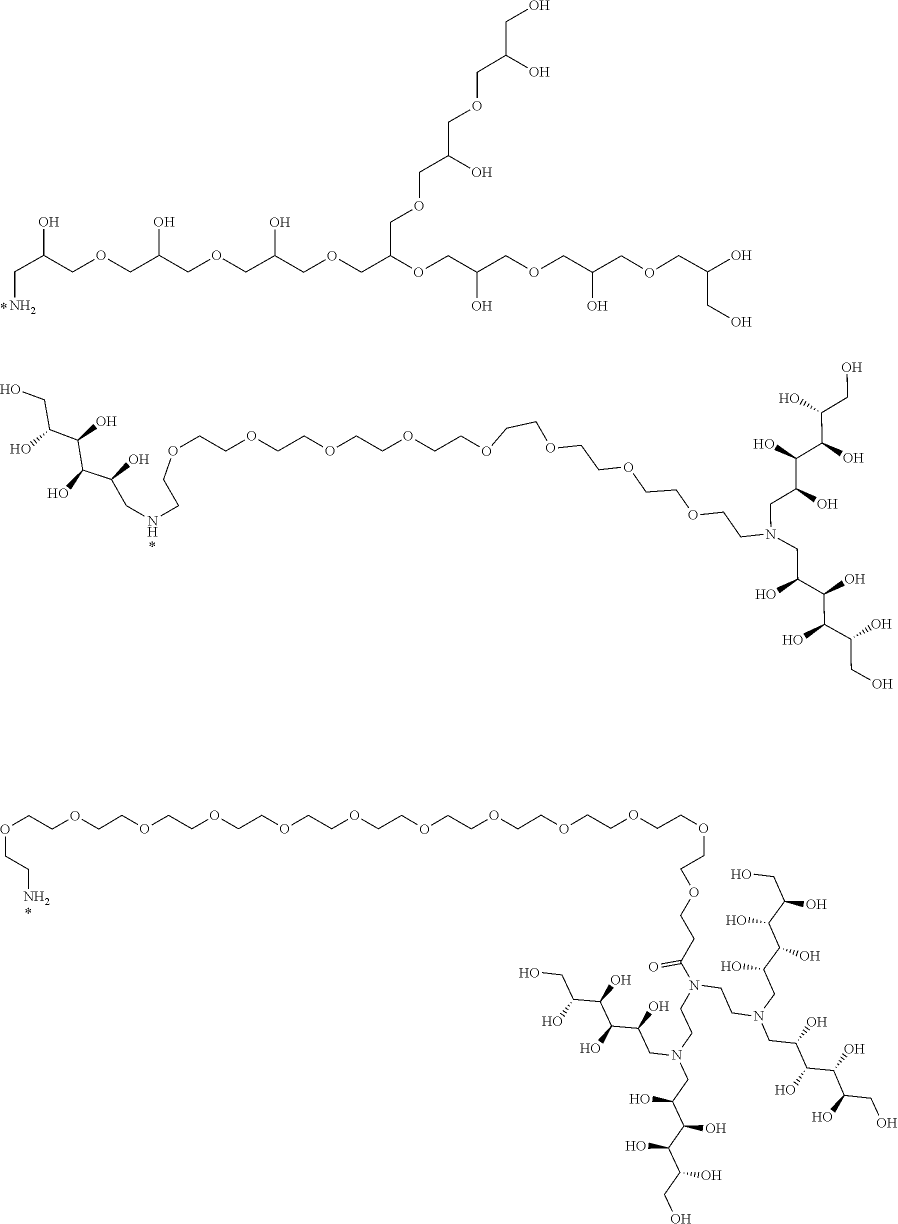 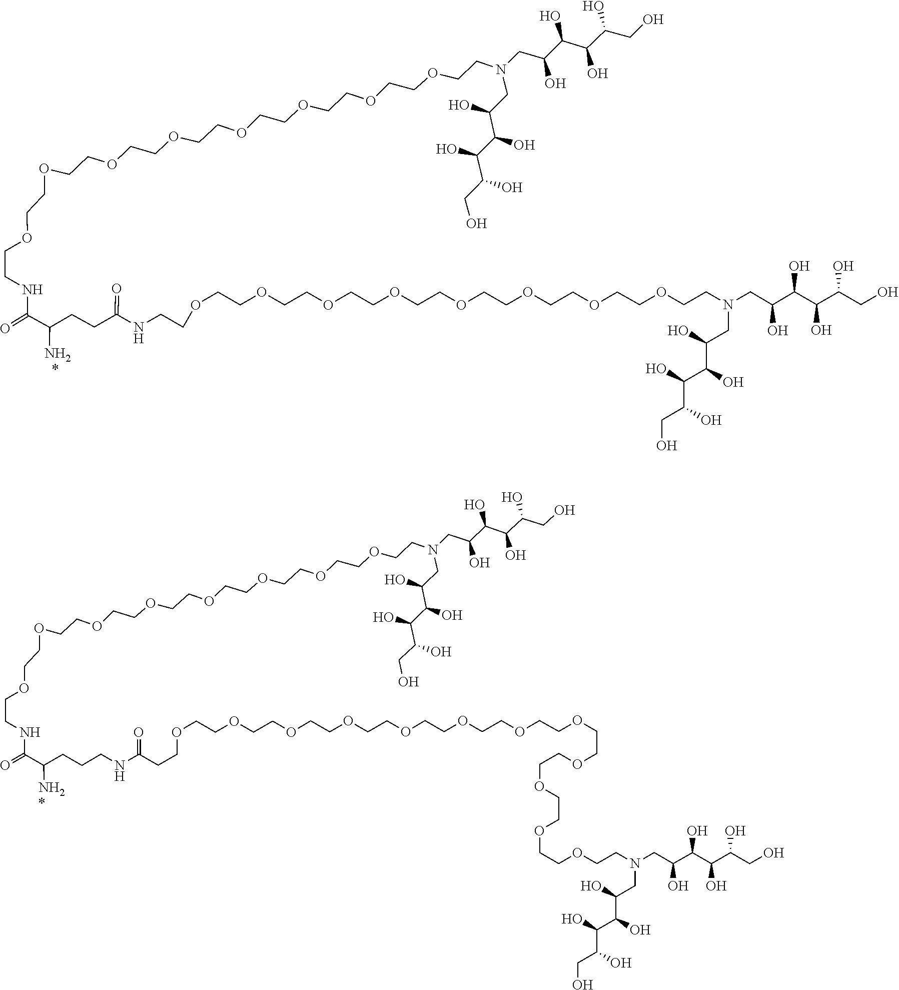 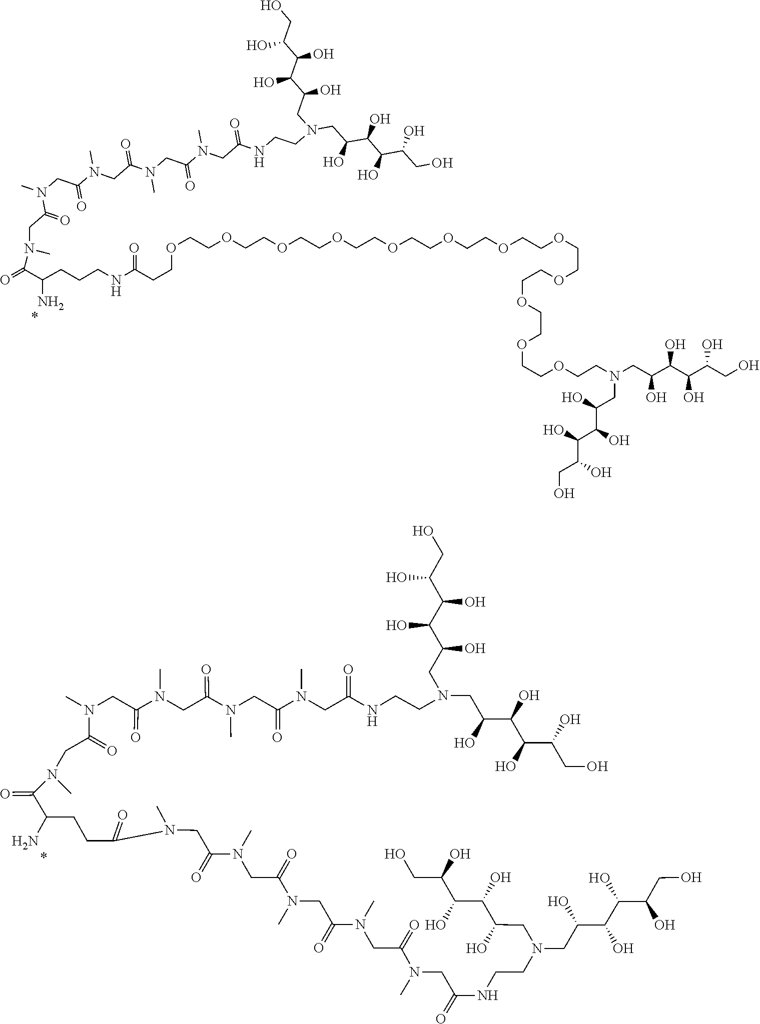 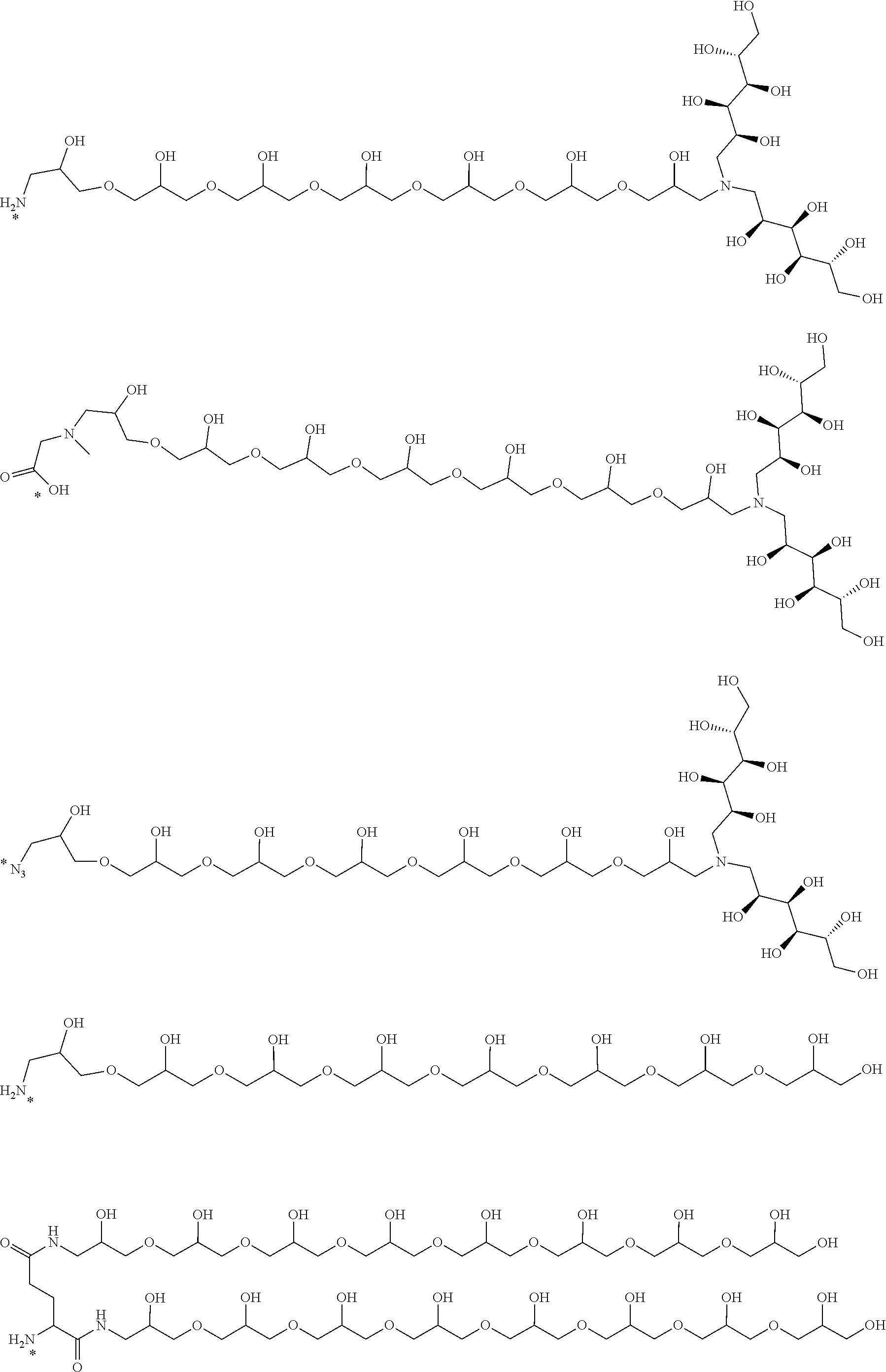 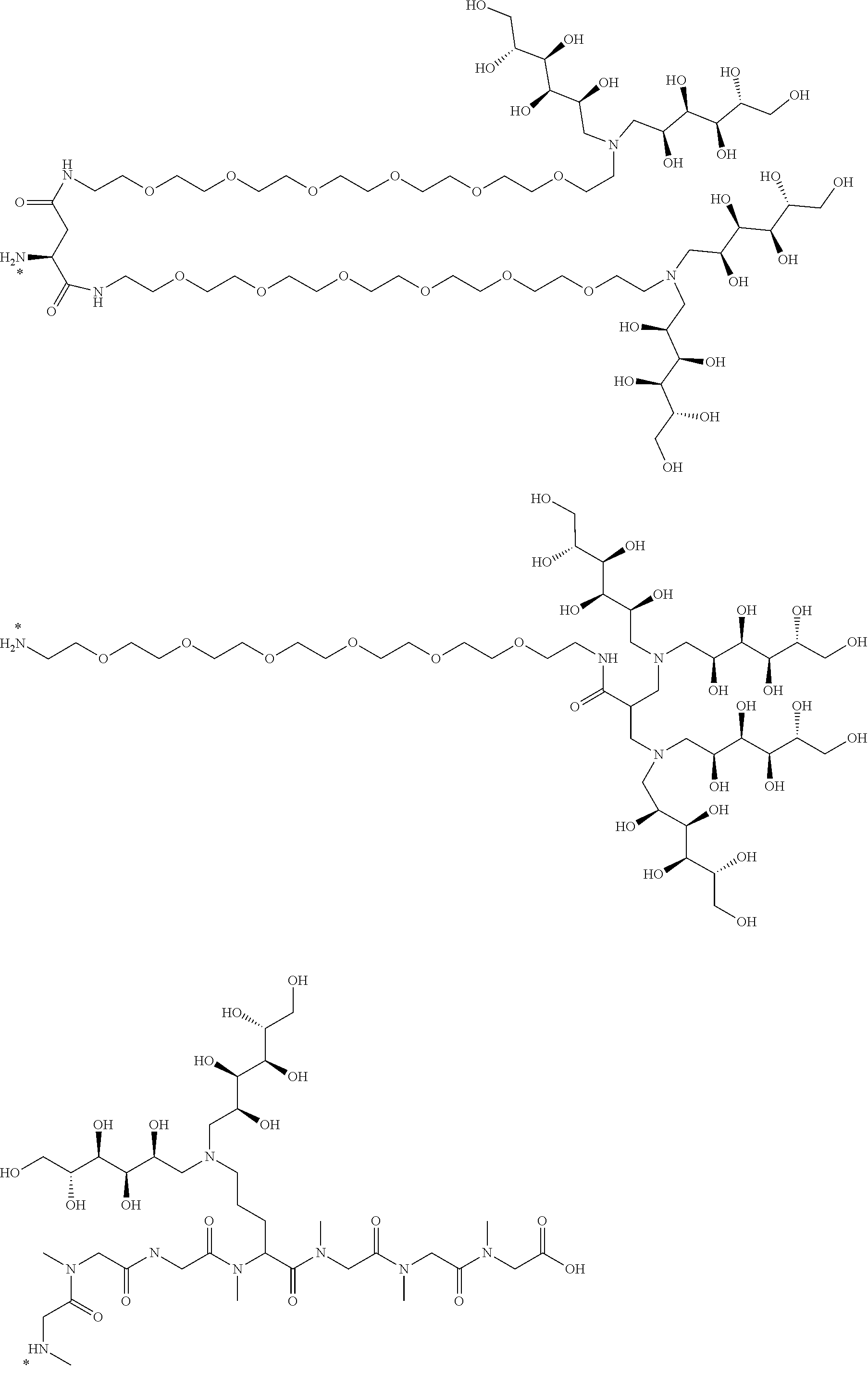 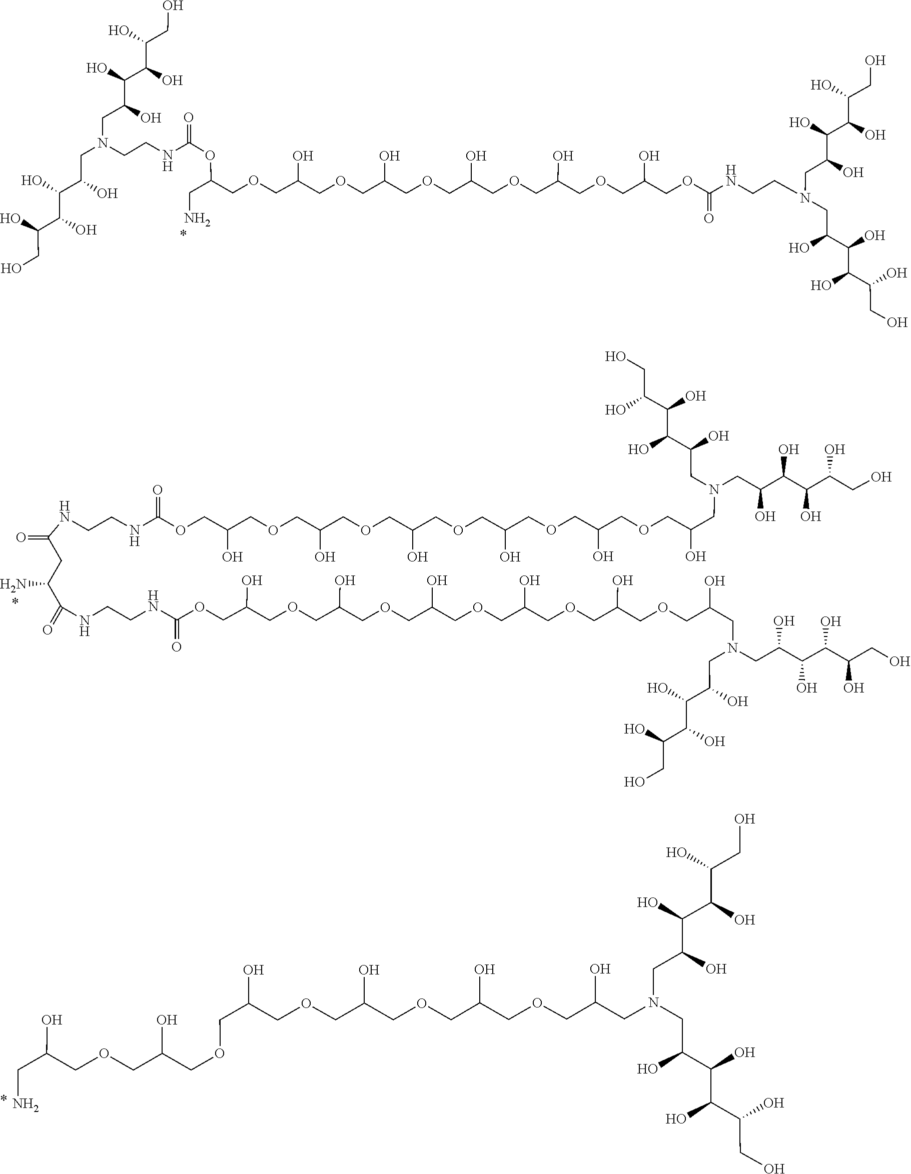 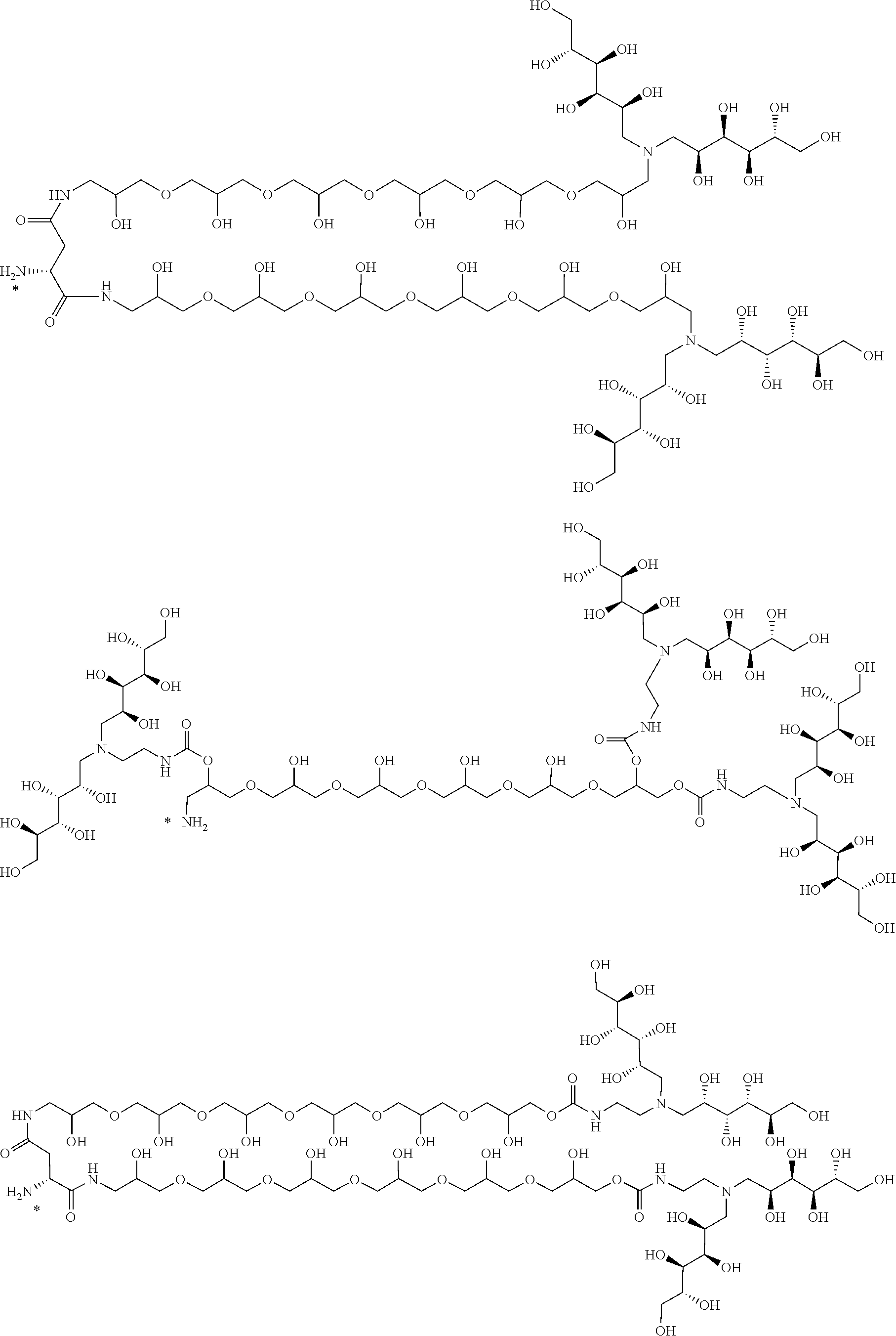 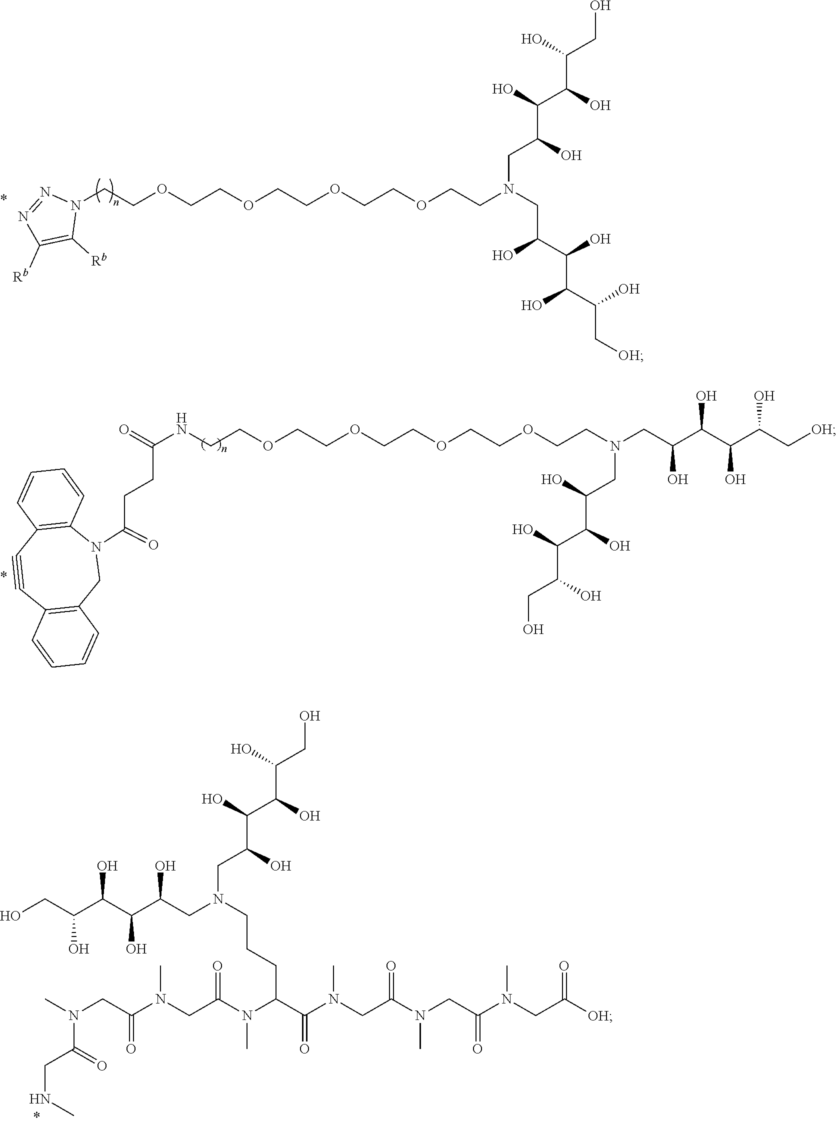 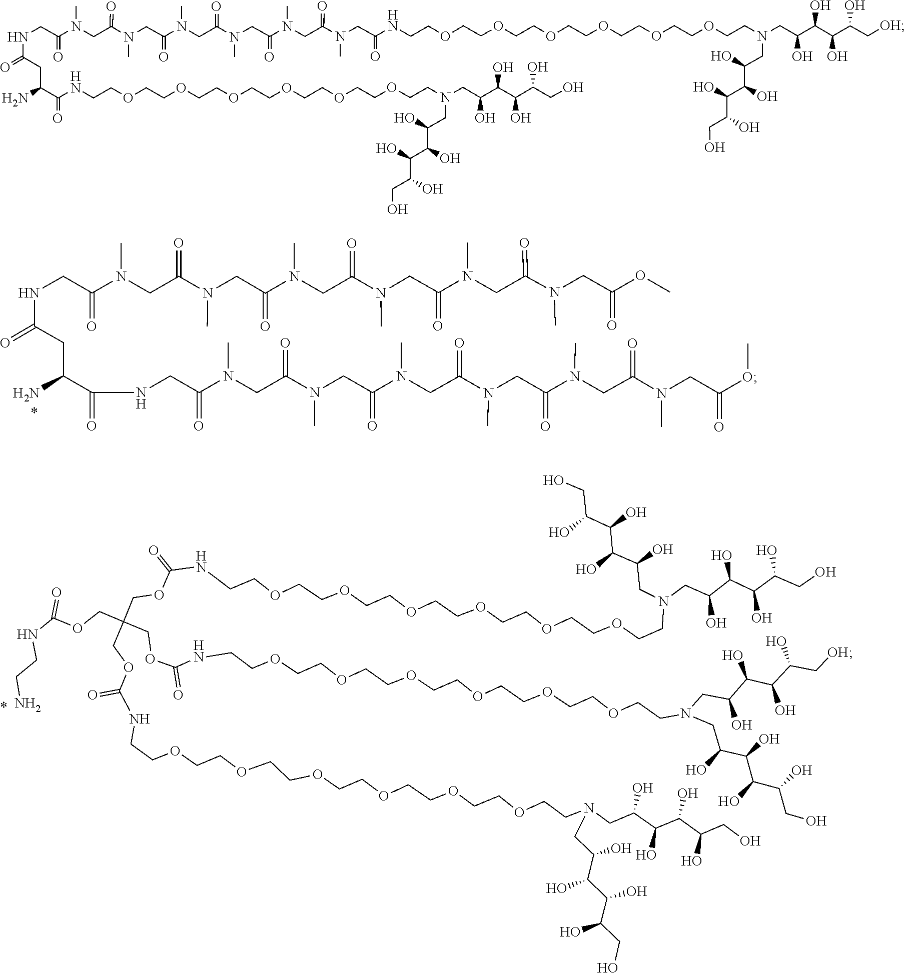 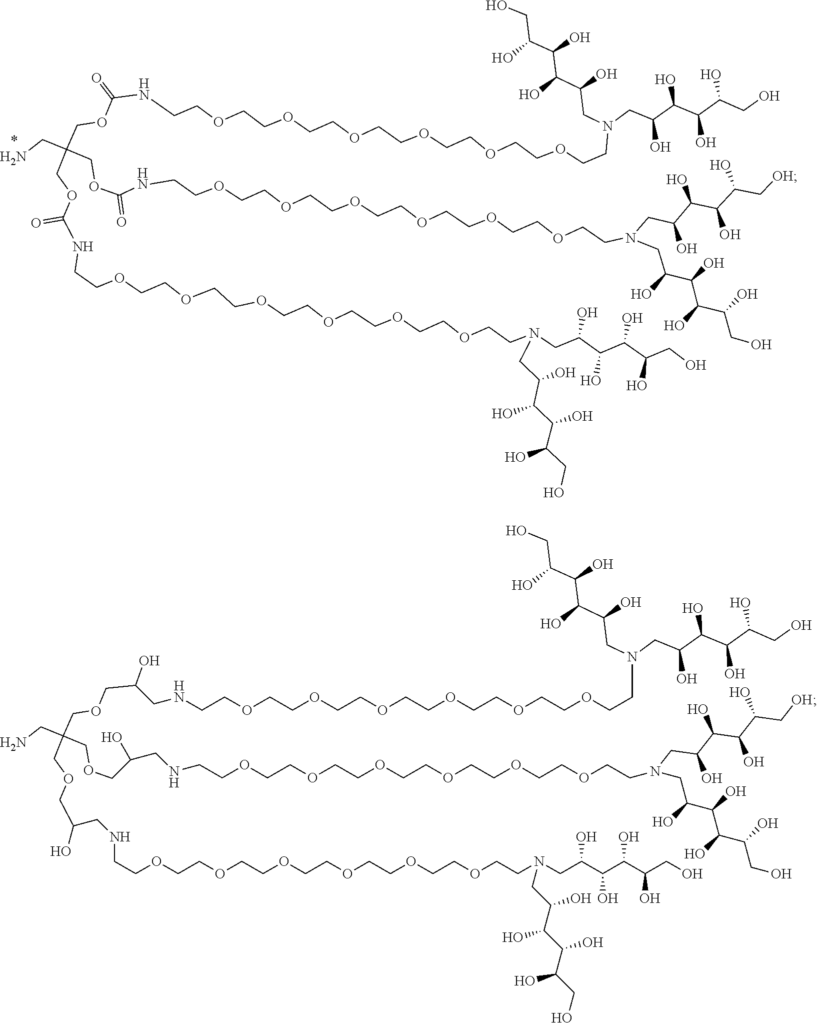 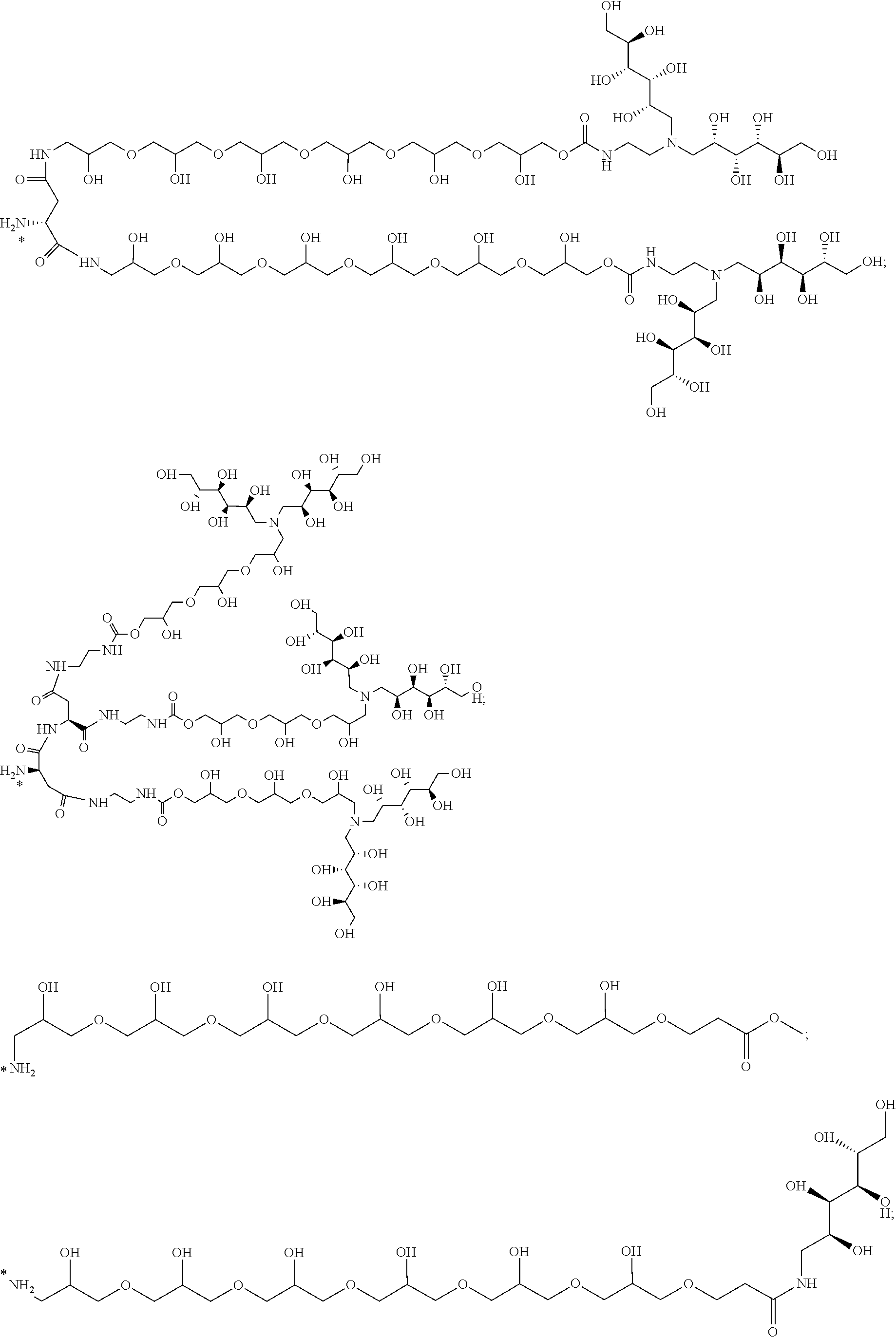 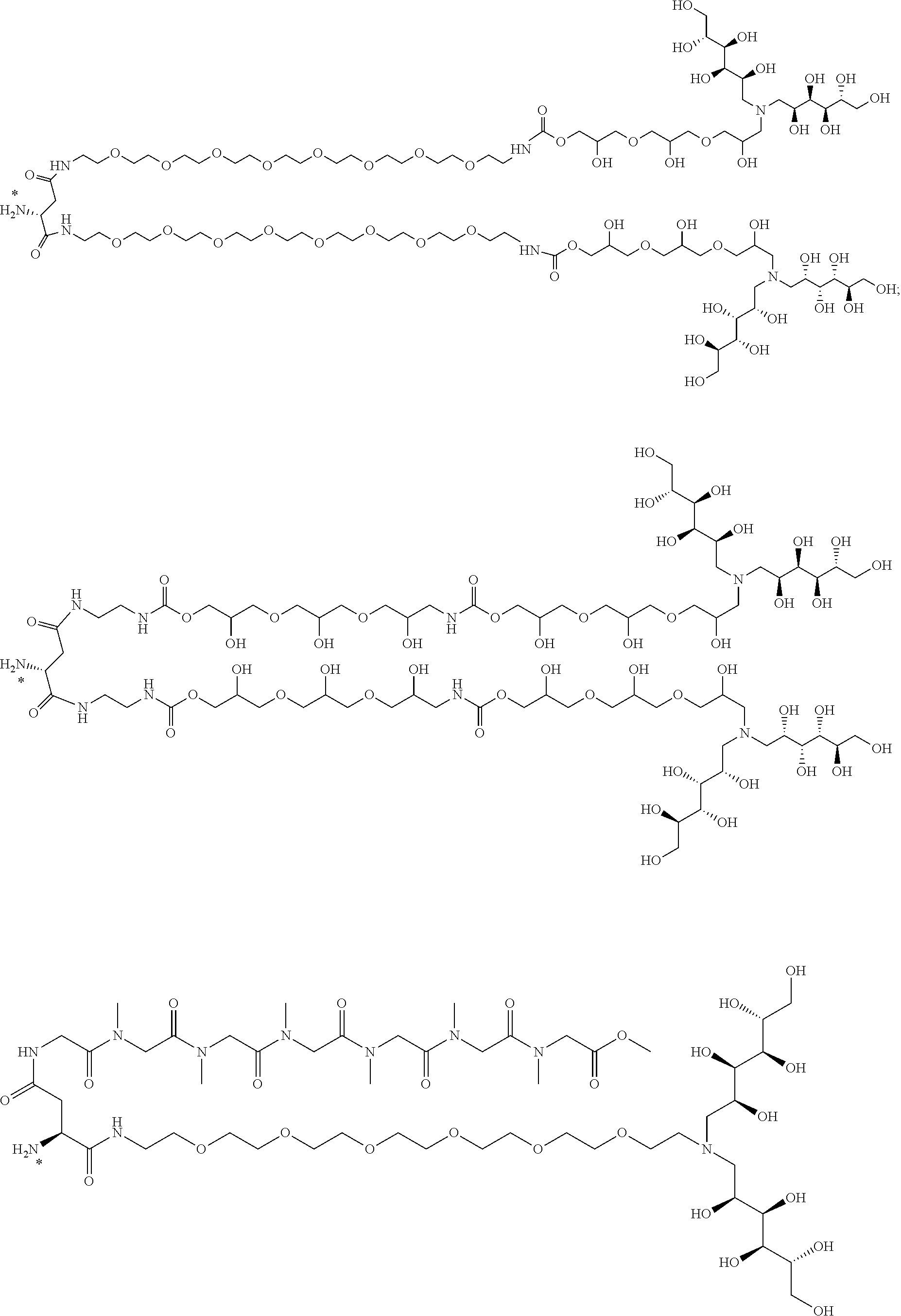 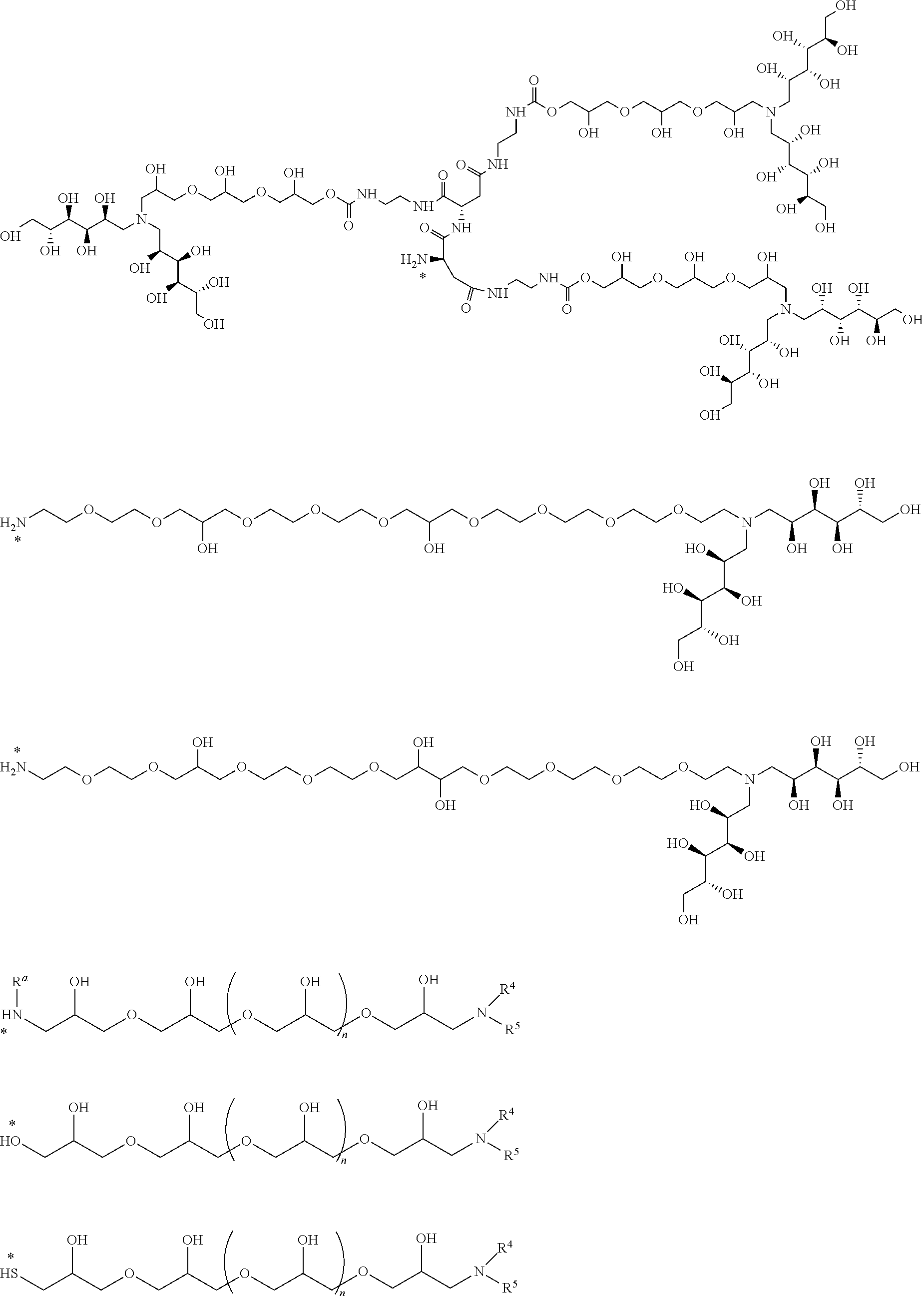 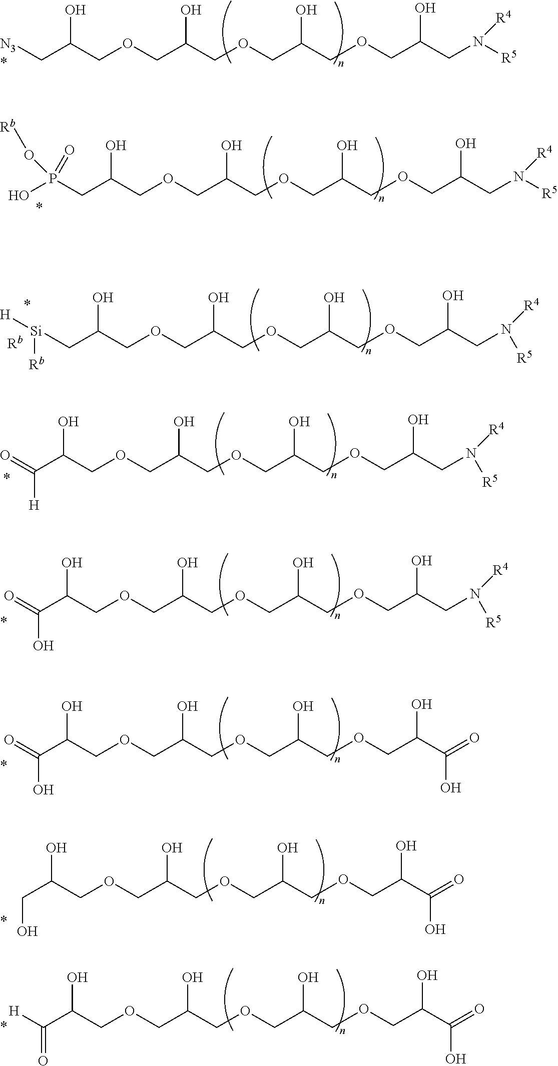 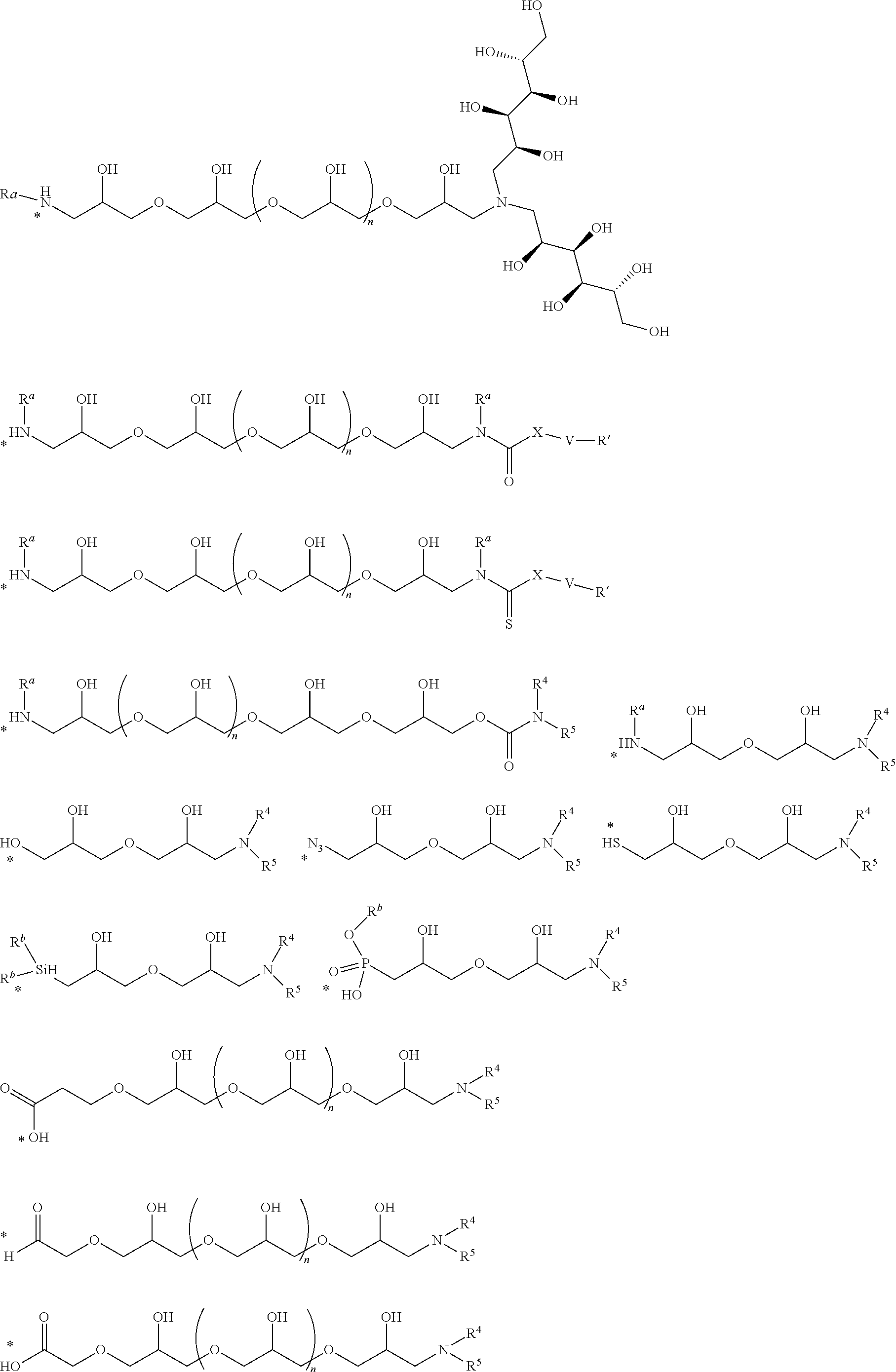 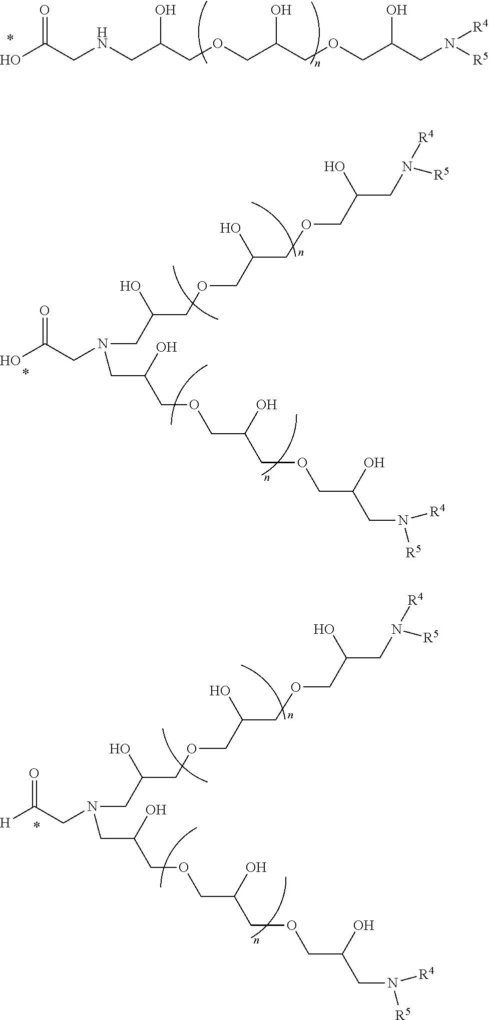 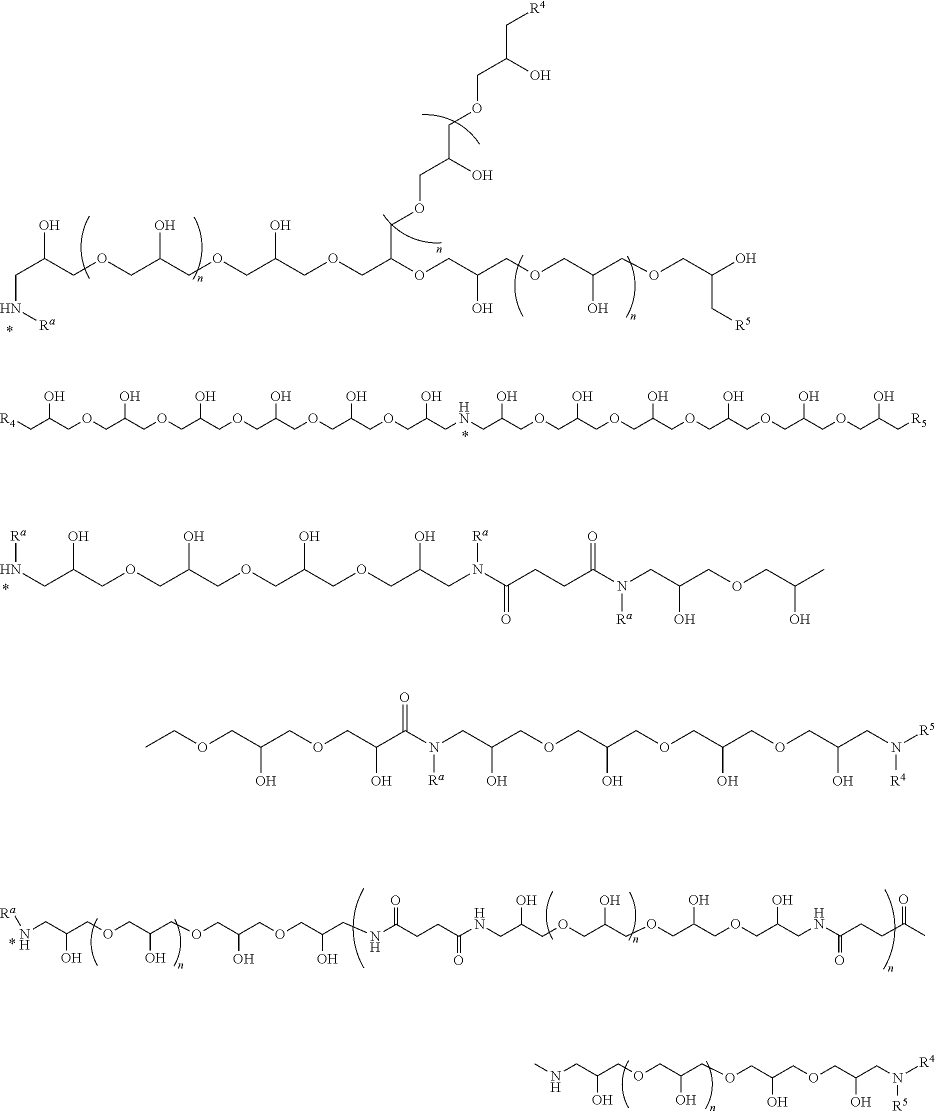 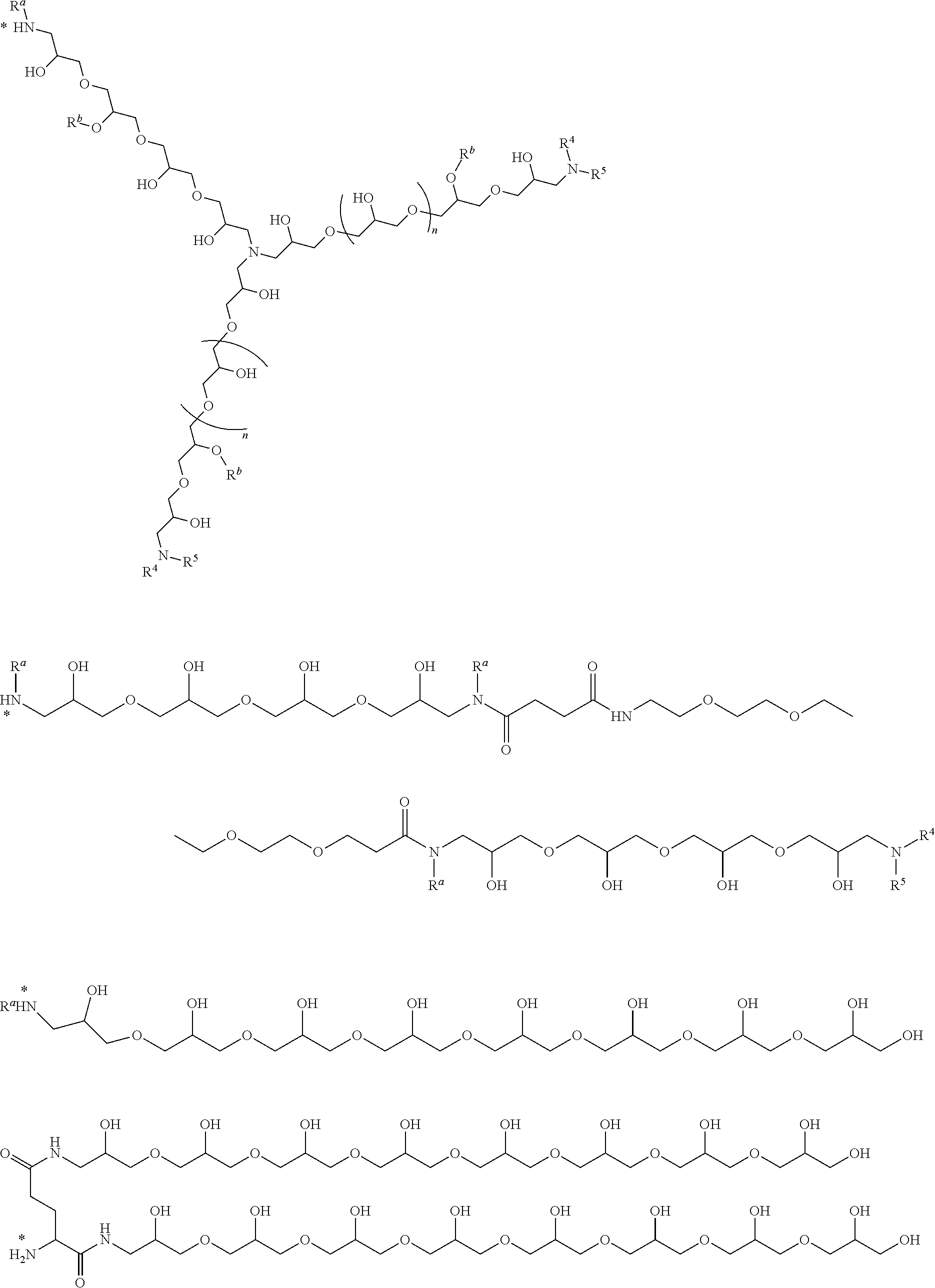 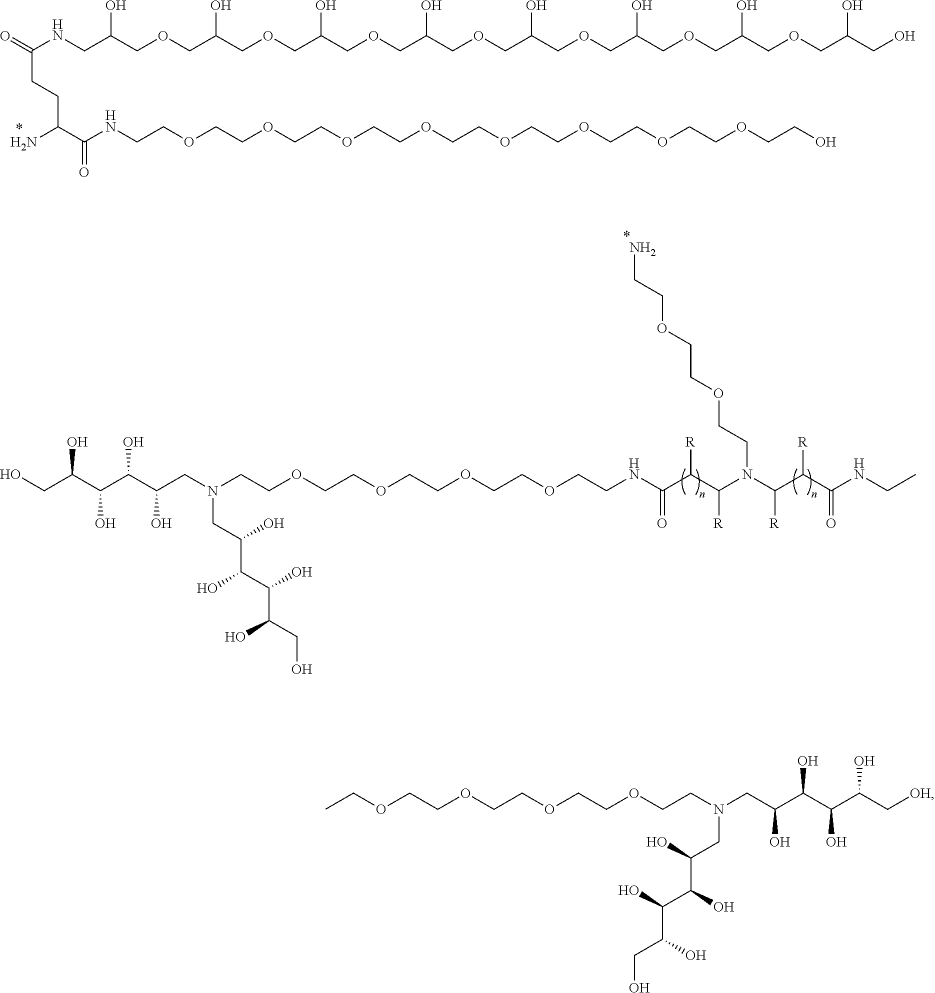 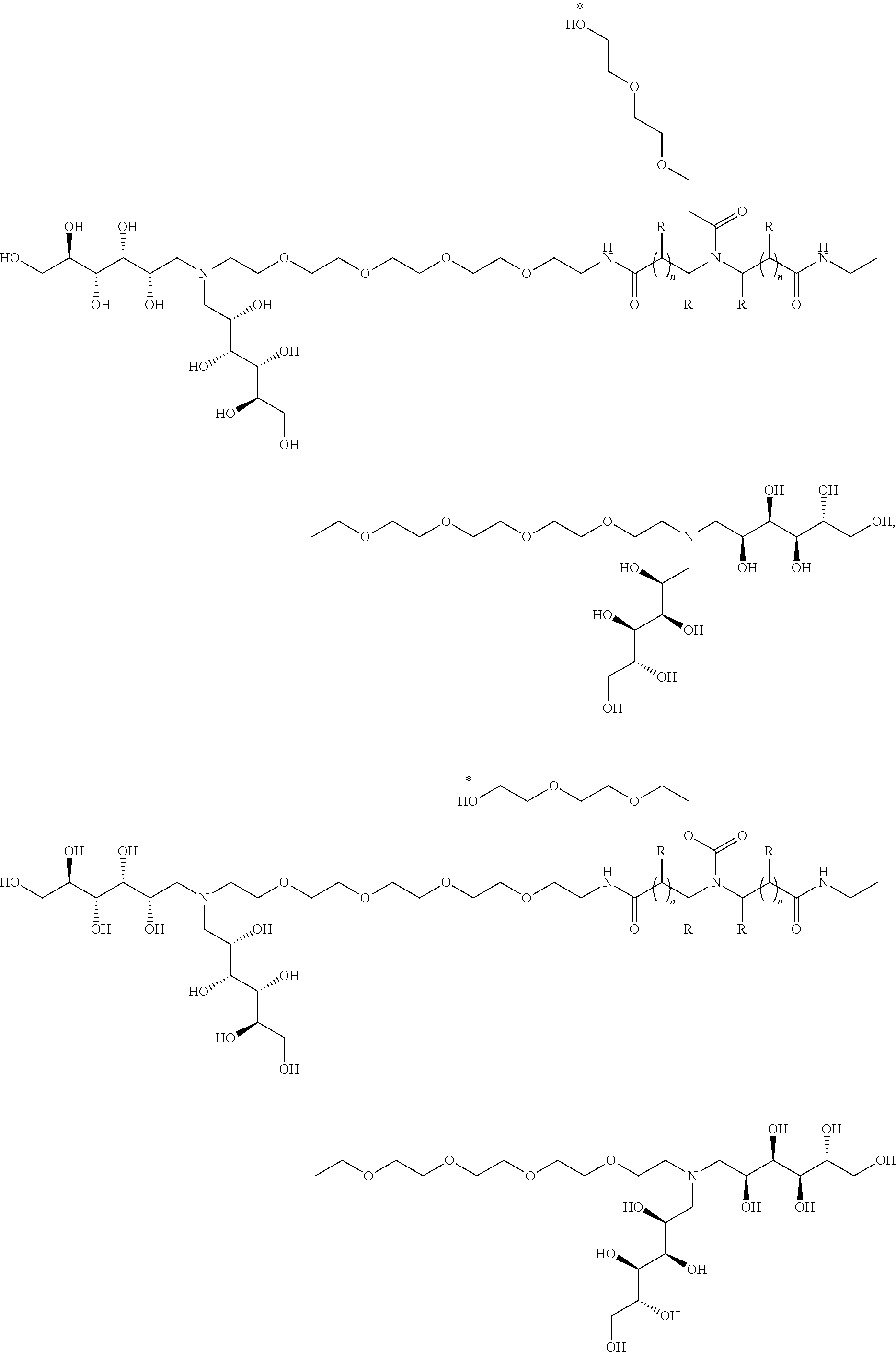
or a stereoisomer thereof, wherein:
(*) indicates the attachment site to an Amino Acid unit;
each R, R^{a} and R is independently H or C₁-C₆ alkyl;
R′ is H, C₁-C₆ alkyl, —N(R⁴)(R⁵) or —CO₂H;
each n is independently 1 to 12;
X is O, NR or —CH₂—;
V is bond or C₁-C₆ alkyl;
one of R⁴ and R⁵ is selected from a H, polyhydroxyl group, substituted polyhydroxyl group, —C(O)-polyhydroxyl group, substituted —C(O)-polyhydroxyl group, a chelator, or —C(O)—R, where R is a Sugar unit of formula (XII) or (XIII); and the other of R⁴ and R⁵ is selected from H, polyhydroxyl group, substituted polyhydroxyl group, —C(O)-polyhydroxyl group, substituted —C(O)-polyhydroxyl group, a chelator, or —C(O)—R, where R is a Sugar unit of formula (XII) or (XIII), and polyethylene glycol, optionally having 1 to 24 ethylene glycol subunits; or —NR⁴R together from a C₃-C₈ heterocycle, and wherein R⁴ and R⁵ are not both H.

54. The Linker compound of claims 1-53, comprising a Polar group having a formula selected from: 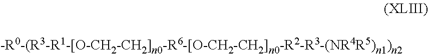
or a stereoisomer or salt thereof, wherein:
R⁰ is a functional group for attachment to a subunit of the Amino Acid unit;
R¹ and R² are each, independently, a bond or C₁-C₆ alkylene;
each R³ is, independently, selected from a bond, C₁-C₁₂ alkylene, —OC₁-C₁₂ alkylene, —C(═O)—, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, C₁-C₁₂alkylene-NH—C(O)—, -heteroarylene, heteroaryl-C₁-C₁₂ alkylene, heteroaryl-C₁-C₁₂ alkylene-C(O)—, and —C(O)NR⁷R⁸, wherein one of R⁷ and R⁸ is H or C₁-C₁₂ alkylene and the other is C₁-C₁₂ alkylene;
R⁴ and R⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, and wherein R⁴ and R⁵ are not both H;
each R⁶ is independently selected from —NR^{a}—, —NR^{a}—C₁-C₆alkylene-NR^{a}—, —NR^{a}—C(O)—NR^{a}—S(O)₂—NR^{a}— or —NR^{a}—C(O)—C₁₋₆alkylene-;
each R^{a} is independently selected from H, C₁-C₆ alkyl, or polyhydroxyl group;
each n0 is independently 2 to 26;
n1 is 1 to 6; and
n2 is 1 to 6; 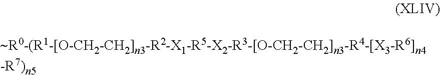
or a stereoisomer or salt thereof, wherein:
R⁰ is a functional group for attachment to a subunit of the Amino Acid unit;
R¹, R², R³ and R⁴ are each, independently, a bond or C₁-C₆ alkylene;
X₁, X₂ and X₃ are each independently —NR^{N}—C(O)— or —C(O)—NR^{N}—;
each R^{N} independently represents H, C₁-C₆ alkyl, or polyhydroxyl group;
R⁵ and R⁶ each independently represent a bivalent polyhydroxyl group;
R⁷ is H, OH or C₁-C₆ alkyl;
each n3 is independently 0 to 26, with the proviso that at least one n3 is 2 to 26;
n4 is 0 to 10; and
n5 is 1 or 2; or 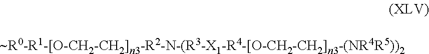
or a stereoisomer or salt thereof, wherein:
R⁰ is a functional group for attachment to a subunit of the Amino Acid unit;
R¹, R³ and R⁴ are each, independently, a bond or optionally-substituted C₁-C₆ alkylene;
each R² is independently a bond, C₁-C₆ alkylene, —C(O)— or —O—C(O)—;
each X₁ is independently —NR^{N}—C(O)— or —C(O)—NR^{N}—;
each R^{N} independently represents H, C₁-C₆ alkyl, or polyhydroxyl group;
R⁴ and R⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, and wherein R⁴ and R⁵ are not both H; and
each n3 is independently 2 to 26.

55. The Linker compound of any one of claims 1-54, comprising a Polar group having one of the following structures prior to attachment to the Amino Acid unit: 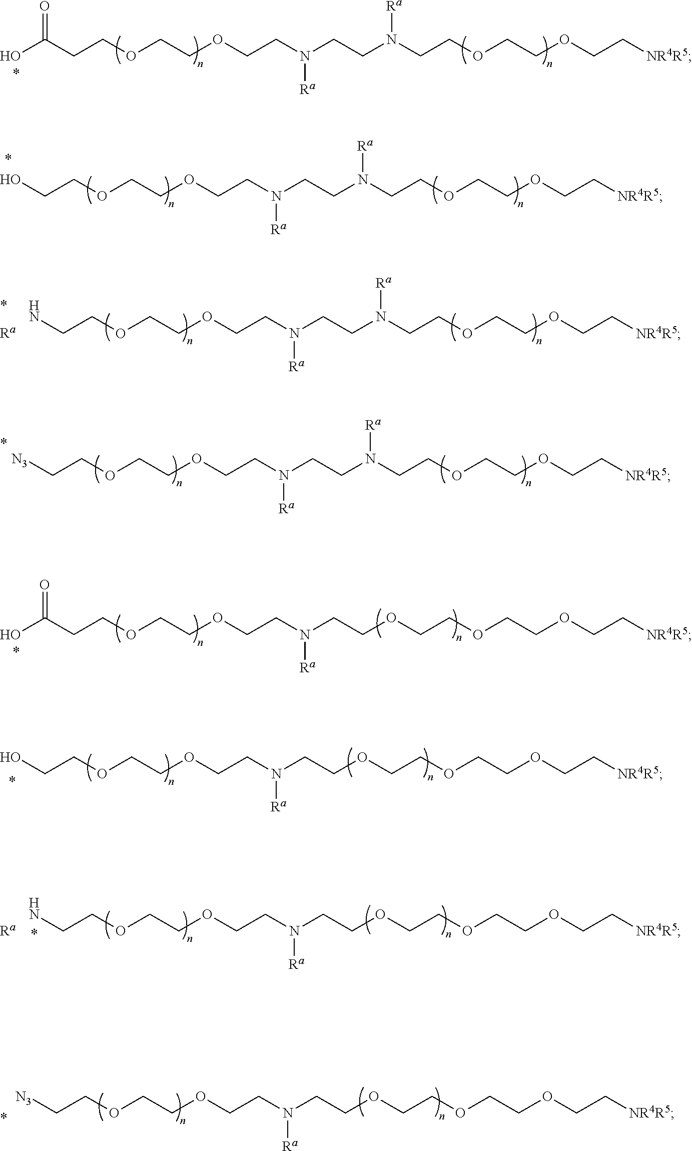 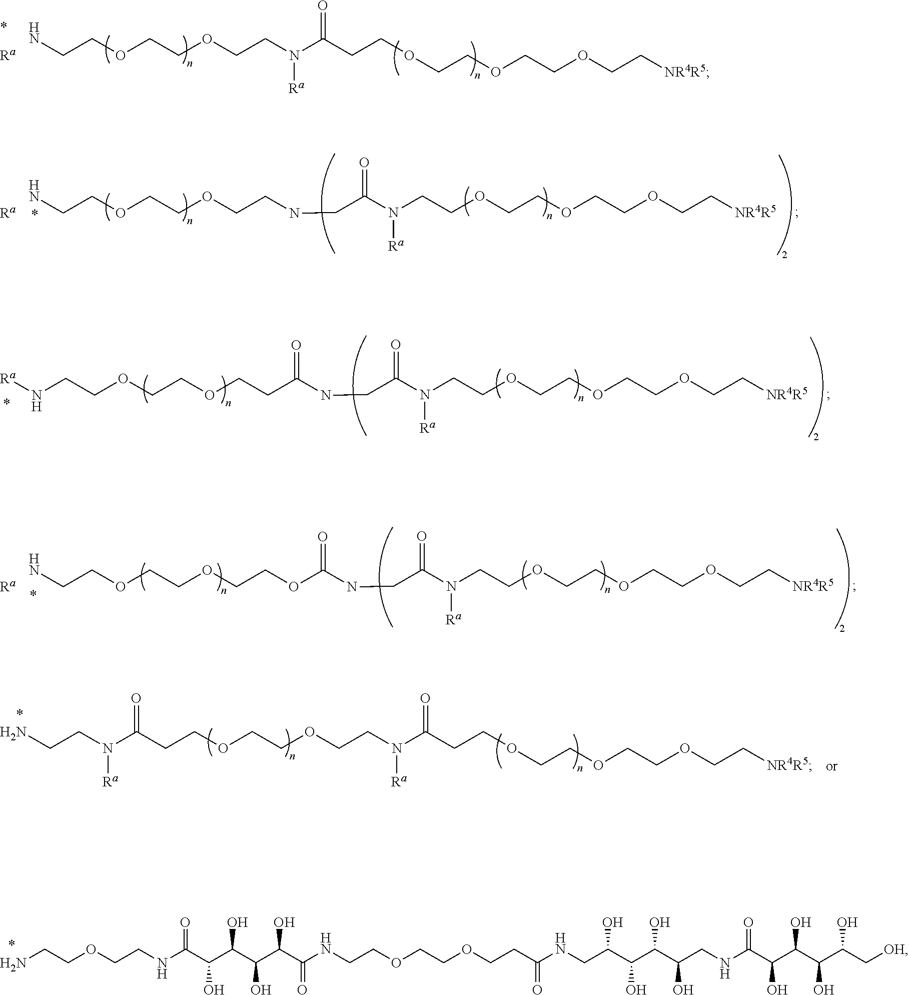
wherein:
(*) indicates the attachment site to an Amino Acid unit;
each R^{a} is independently H, alkyl or polyhydroxyl group;
R⁴ and R⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, and wherein R⁴ and R⁵ are not both H; and
each n is independently 1 to 12.

56. The Linker compound of any one of claims 1-55, comprising a Polar group having a formula selected from: 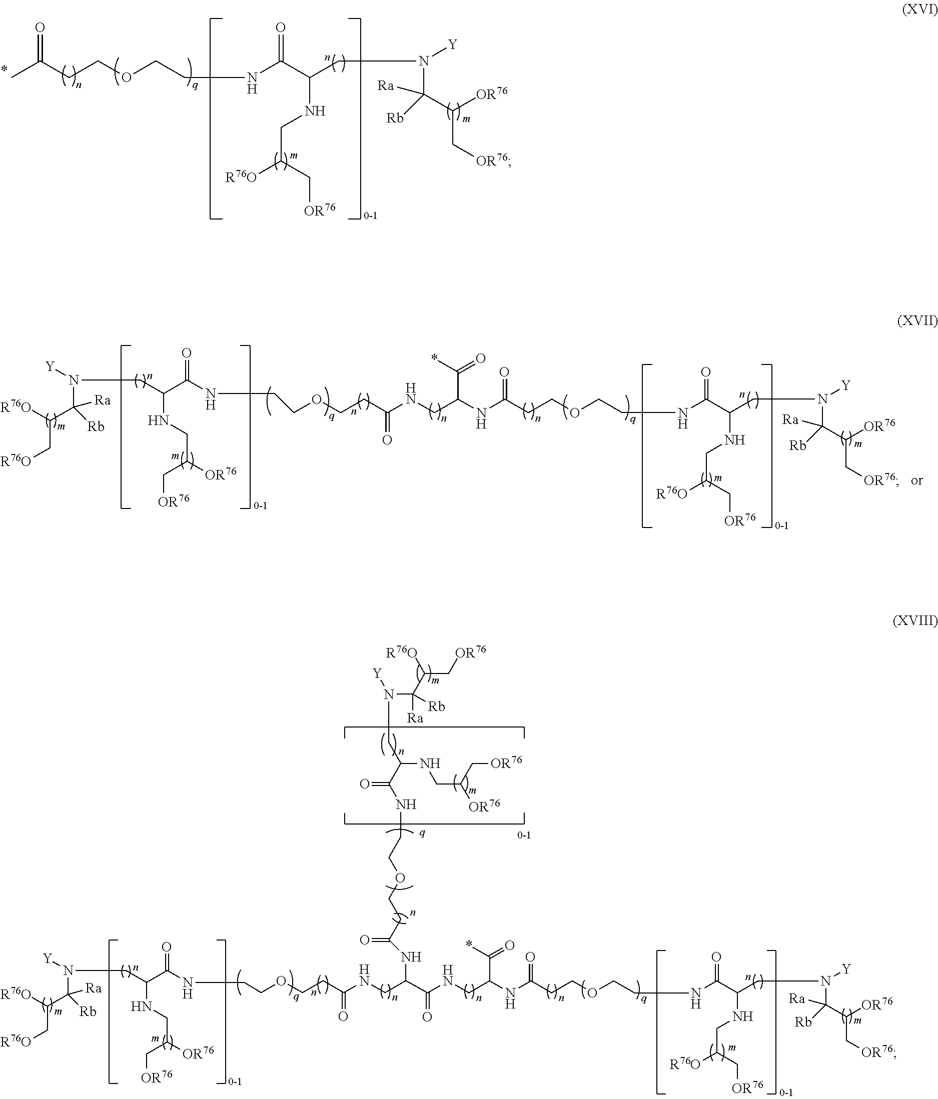
or a stereoisomer or salt thereof, wherein:
each Y is independently R⁷⁶ or 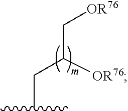
each R⁷⁶ is independently H, acetyl, —P(═O)(OH)₂, or —(CH₂)ᵥ—O—S(═O)₂(OH);
each Rₐ and R_{b} is independently H or Rₐ and R_{b} are taken together with the carbon to which they are attached to form an oxo group;
each q is independently 2-26;
each m is independently 1 to 4;
each n is independently 1 to 4;
each v is independently 1 to 6; and
each * is an attachment to an Amino Acid unit.

57. The Linker compound of any one of claims 1-56, comprising a Polar group having a formula selected from: 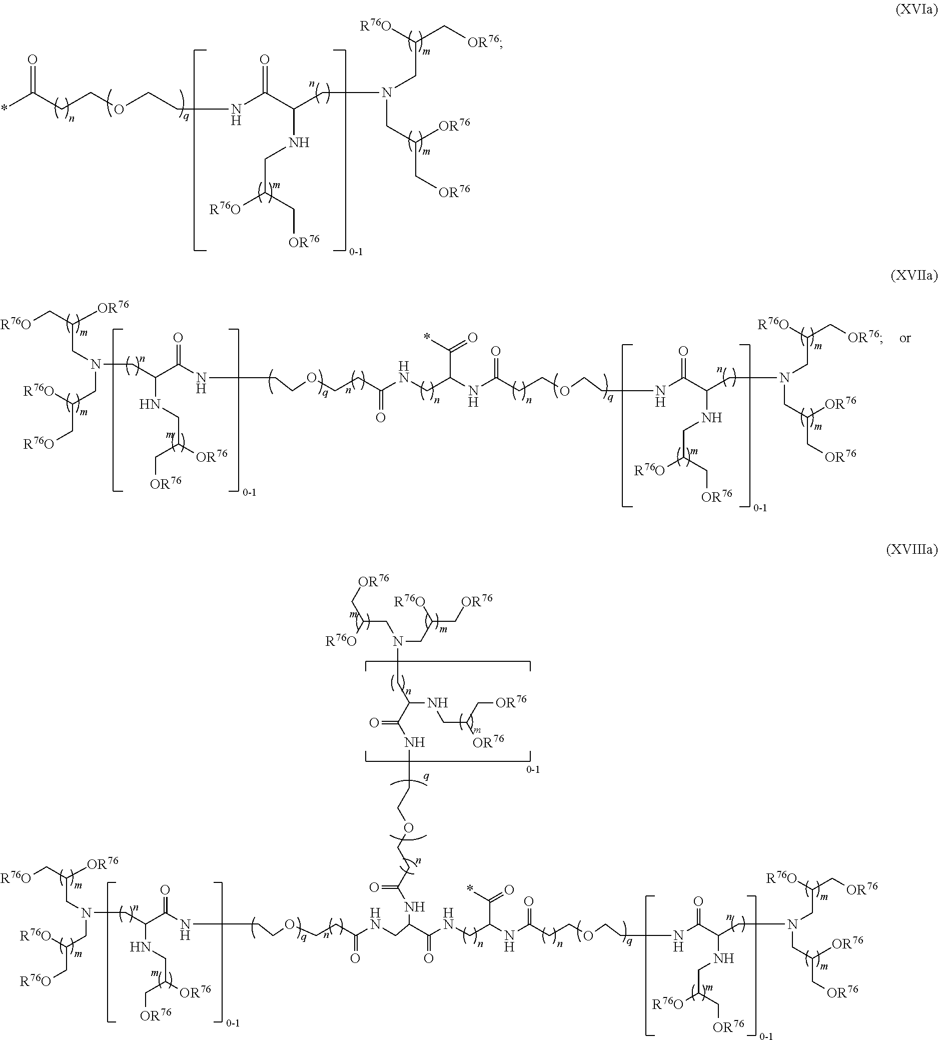
or a stereoisomer or salt thereof, wherein:
each R^{H} is independently H, acetyl, —P(═O)(OH)₂, or —(CH₂)S(═O)₂(OH);
each q is independently 2-26;
each m is independently 1 to 4;
each n is independently 1 to 4;
each v is independently 1 to 6; and
each * is an attachment to an Amino Acid unit.

58. The Linker compound of any one of claims 1-57, comprising a Polar group having a formula selected from: 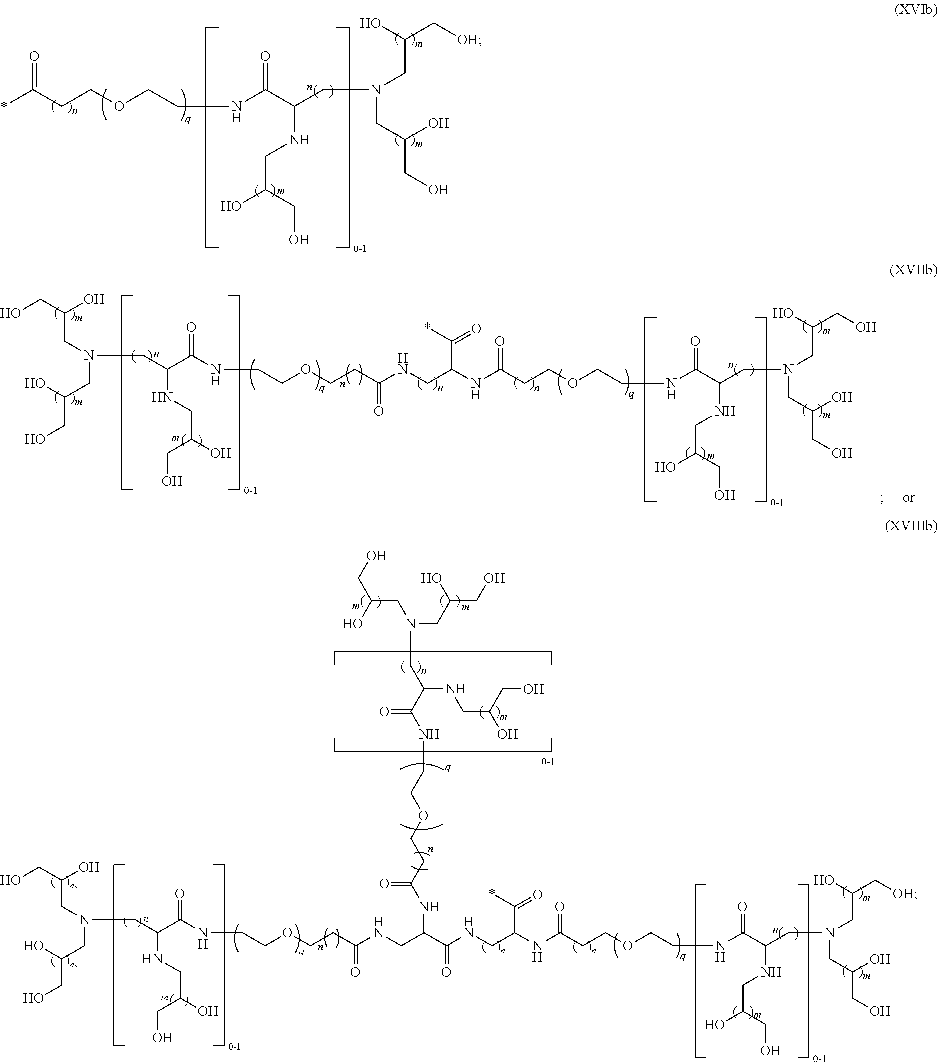
or a stereoisomer or salt thereof, wherein:
each q is independently 2-26;
each m is independently 1 to 4;
each n is independently 11 to 4; and
each * is an attachment to an Amino Acid unit.

59. The Linker compound of claim 58, wherein Y is R⁷⁶.

60. The Linker compound of claim 58, wherein Y is

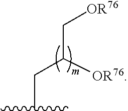

61. The Linker compound of claim 58, wherein each Rₐ and R_{b} is independently H.

62. The Linker compound of claim 58, wherein Rₐ and R_{b} are taken together with the carbon to which they are attached to form an oxo group.

63. The Linker compound of any one of claims 56-58, wherein q is 10-20.

64. The Linker compound of any one of claims 56-58, wherein q is 12.

65. The Linker compound any one of claims 1-64, wherein the Polar group has one of the following structures prior to attachment to the Amino Acid unit: 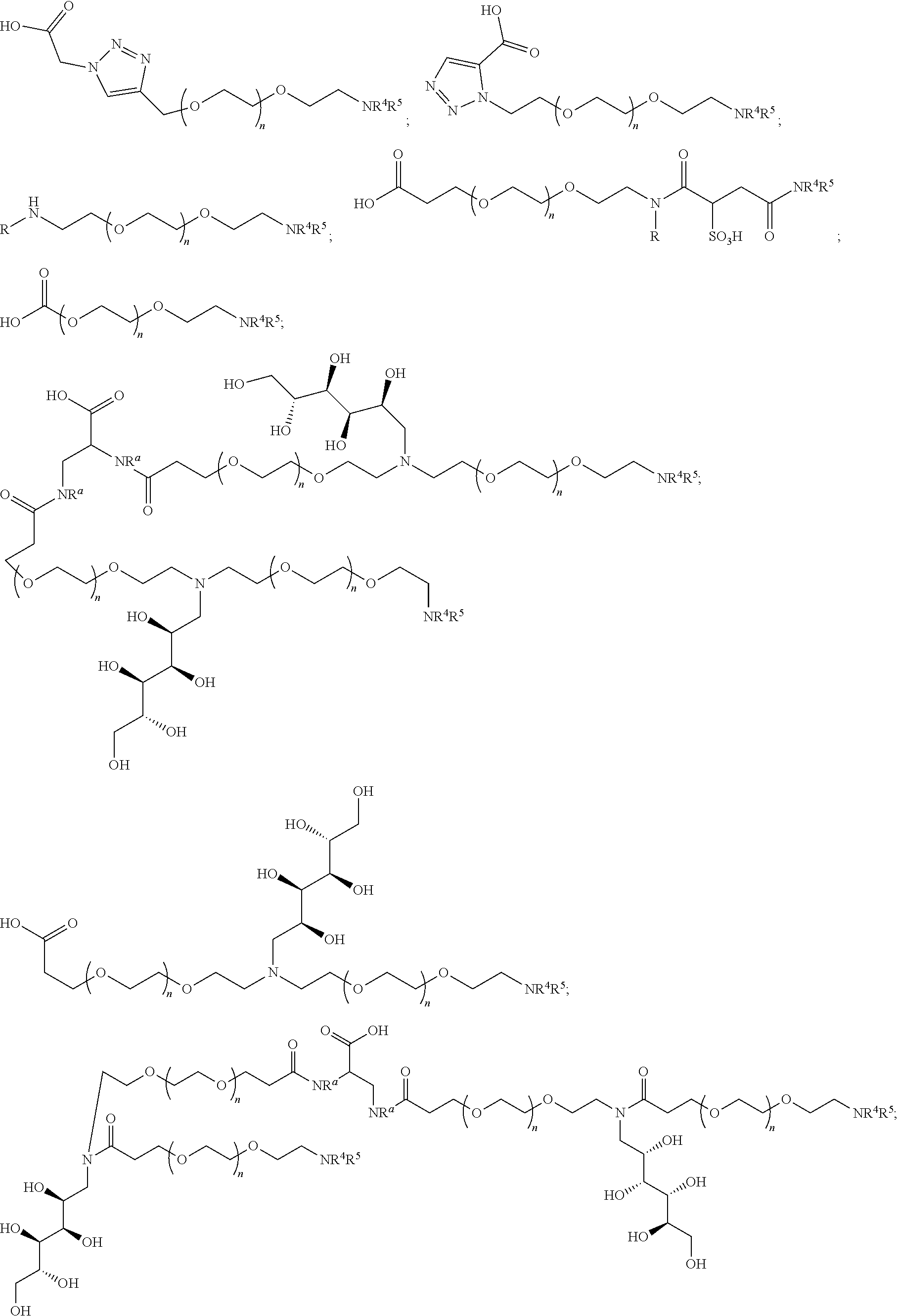 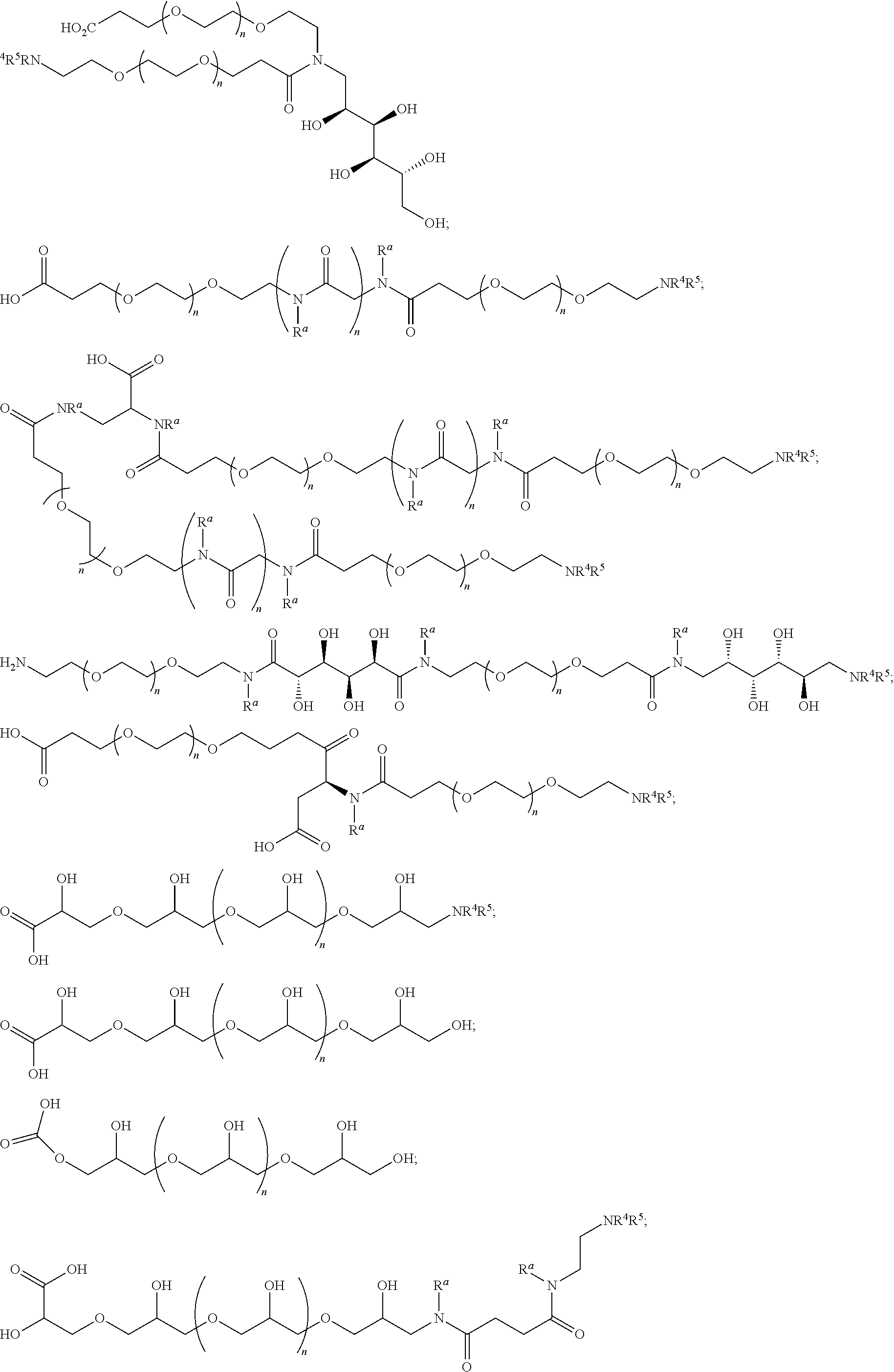 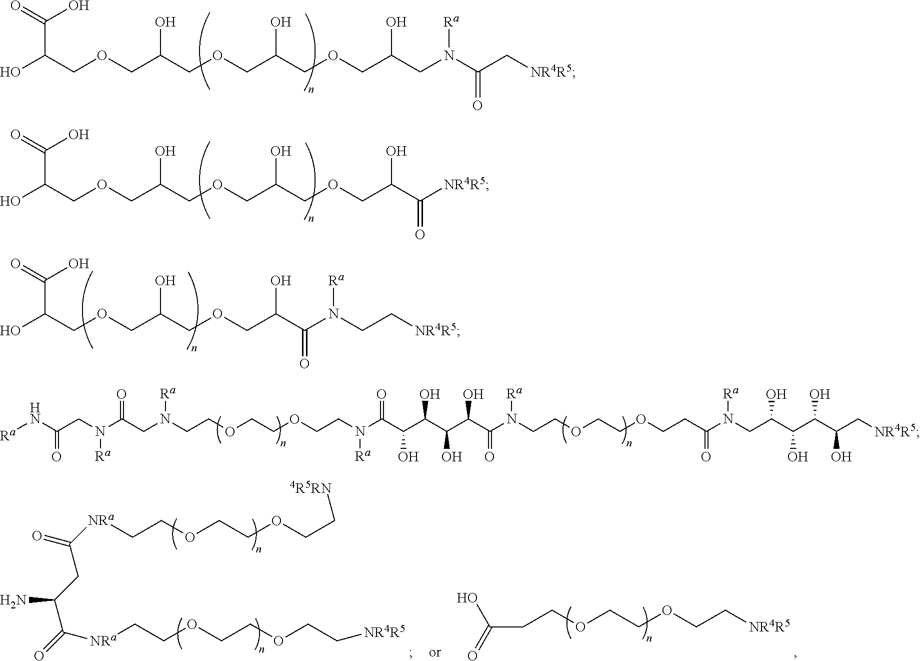
or a stereoisomer thereof, wherein R^{a} is H or C₁₋₆ alkyl and n is 1-20.

66. The Linker compound of any one of claims 1-65, wherein the Polar group has one of the following structures prior to attachment to the Amino Acid unit: 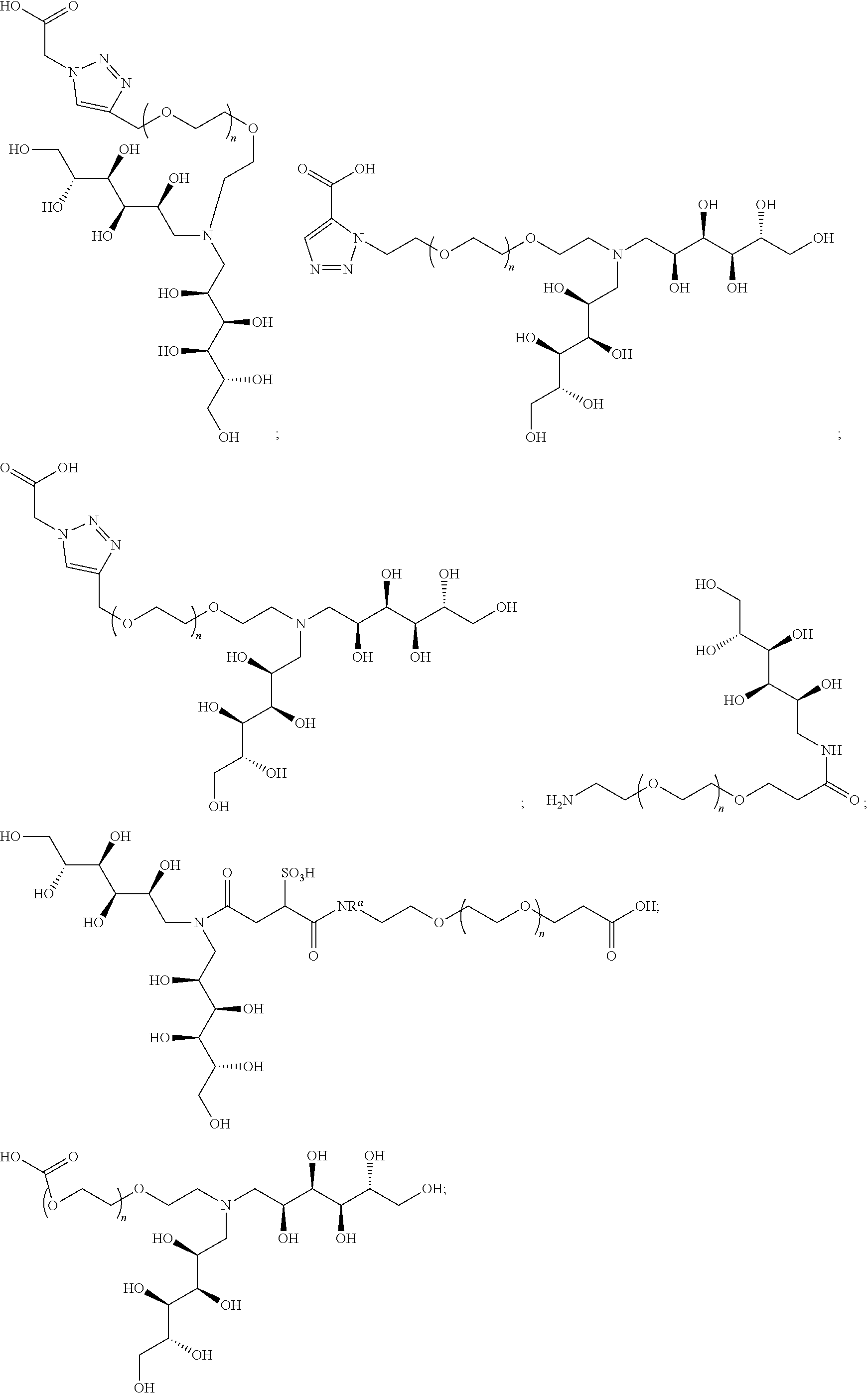 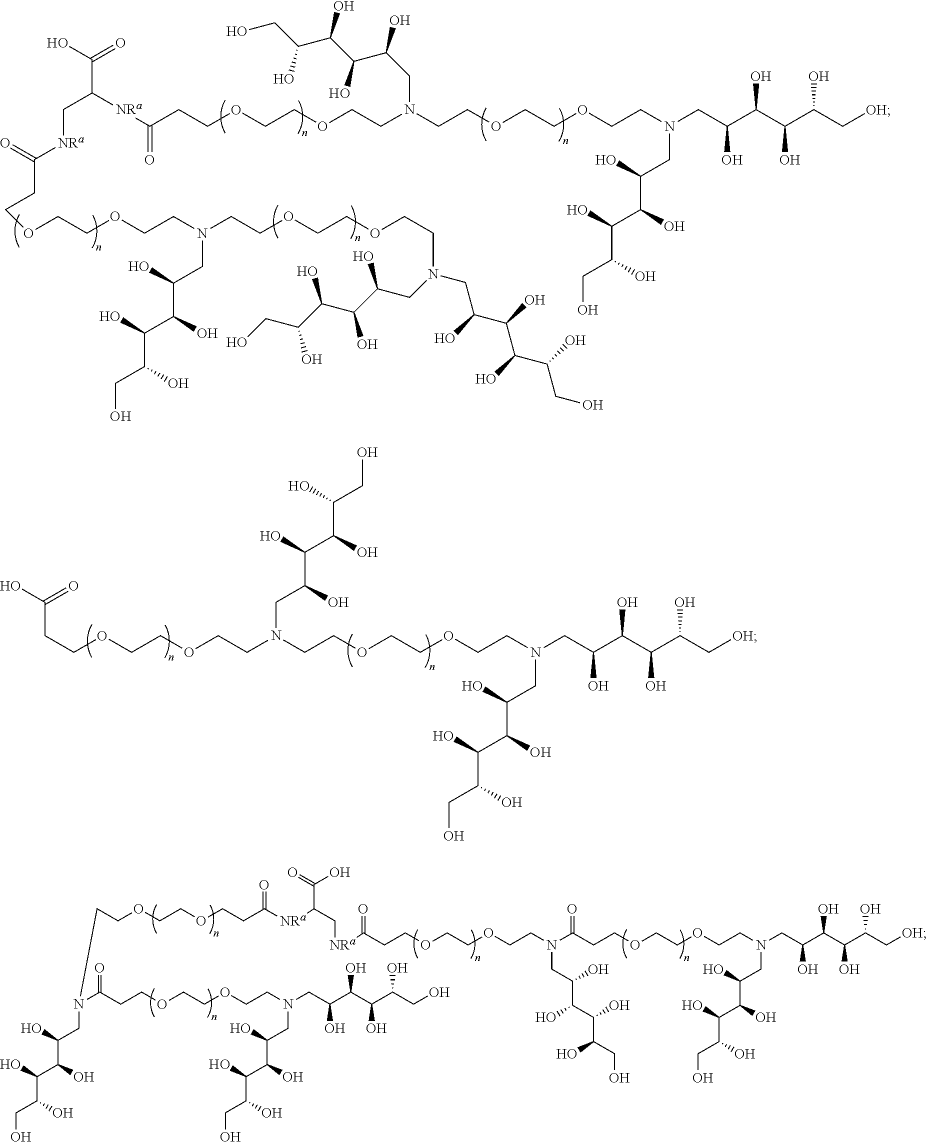 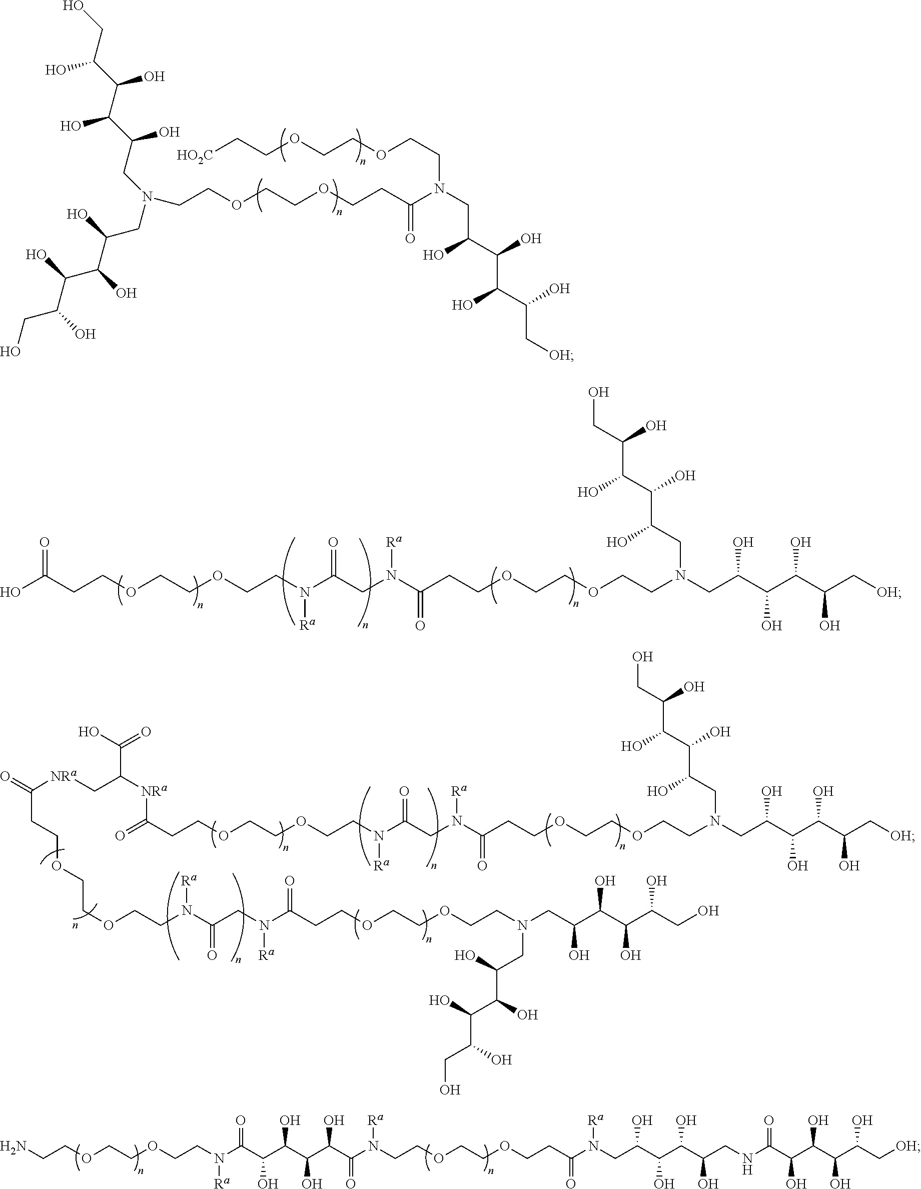 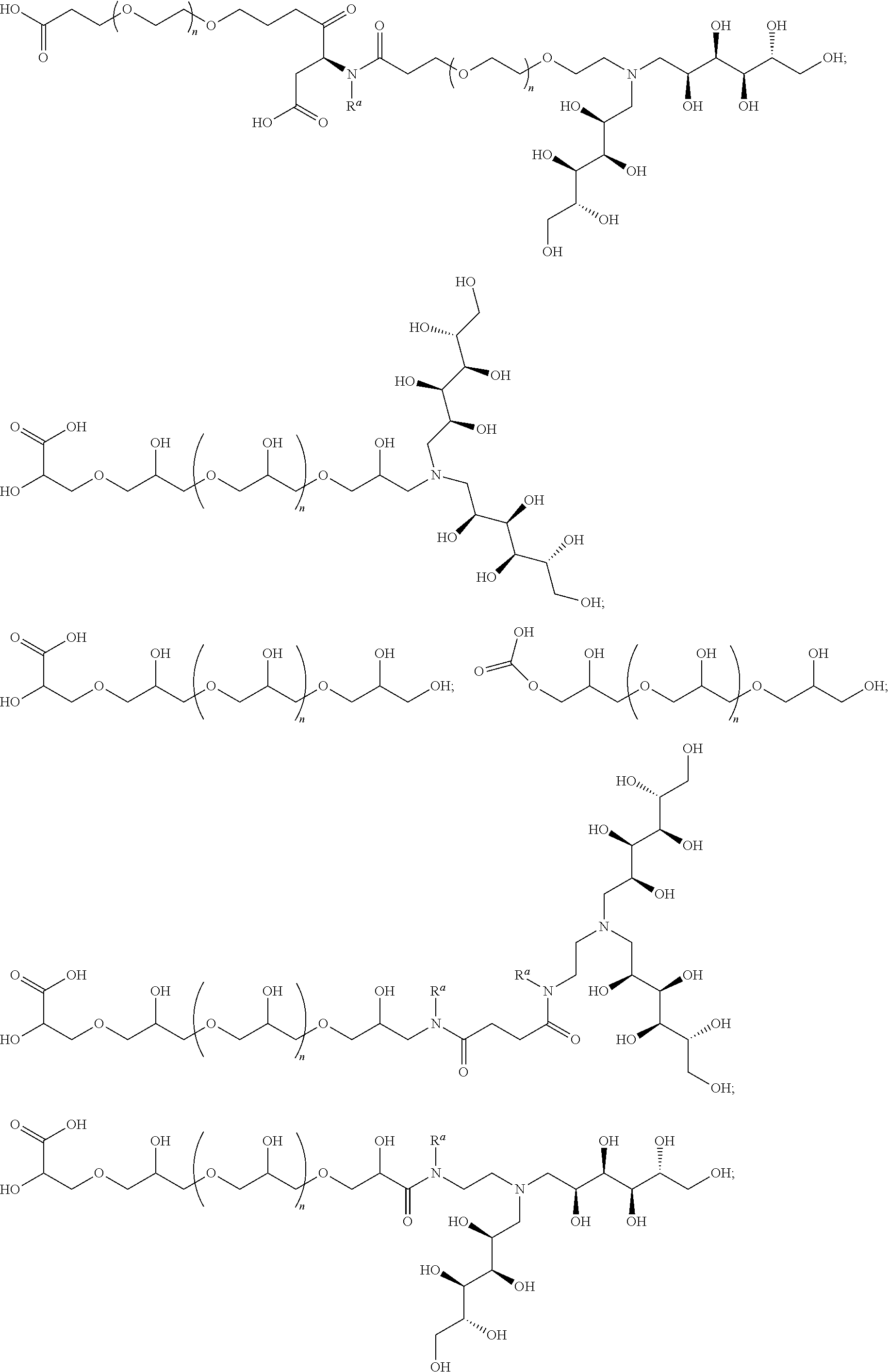 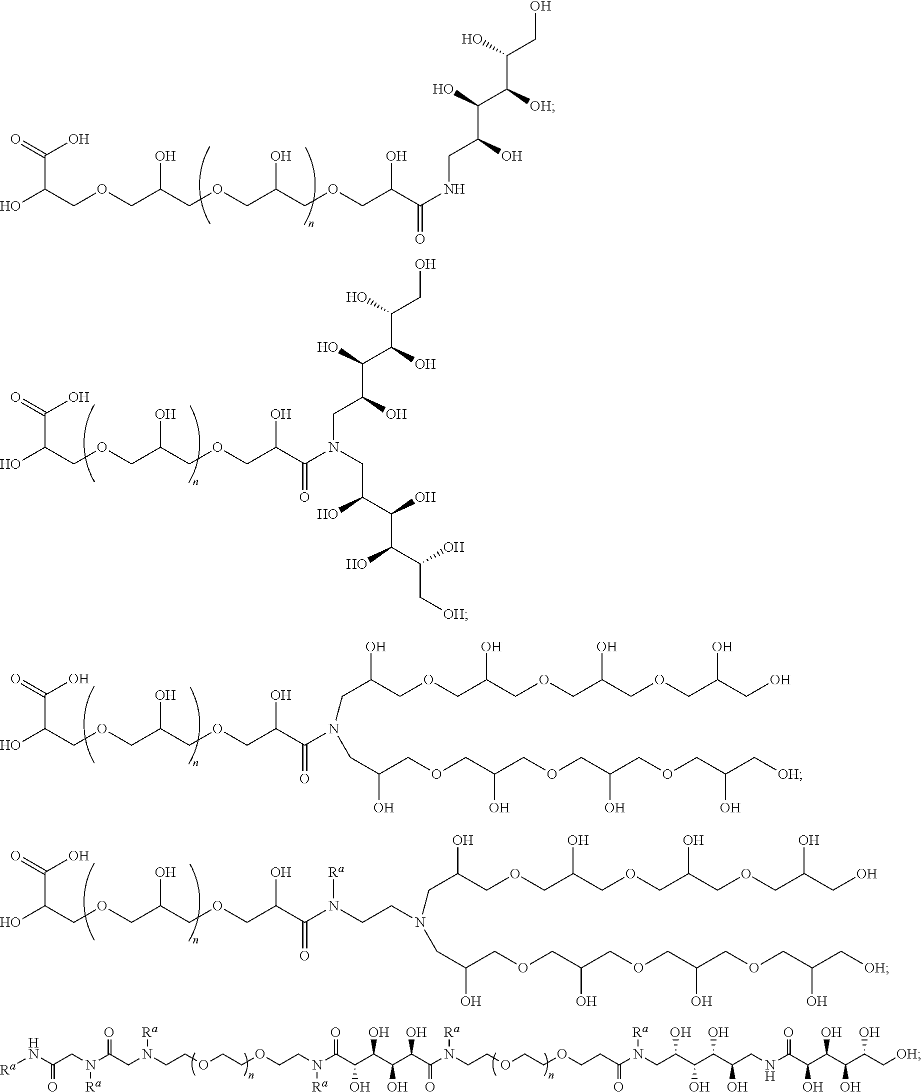 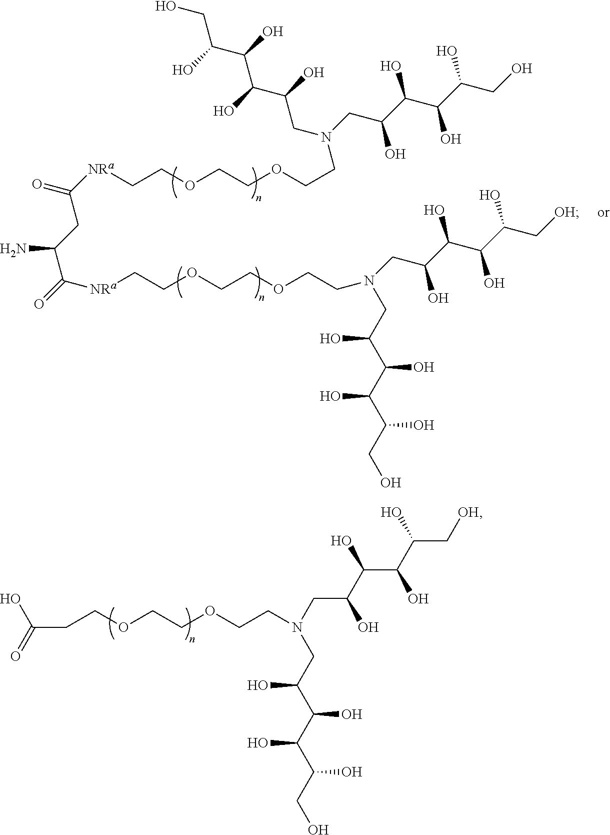
or a stereoisomer thereof, wherein R^{a} is H or C₁₋₆ alkyl and n is 1-20.

67. The Linker compound of any one of claims 1-66, wherein the Polar group has one of the following structures prior to attachment to the Amino Acid unit: 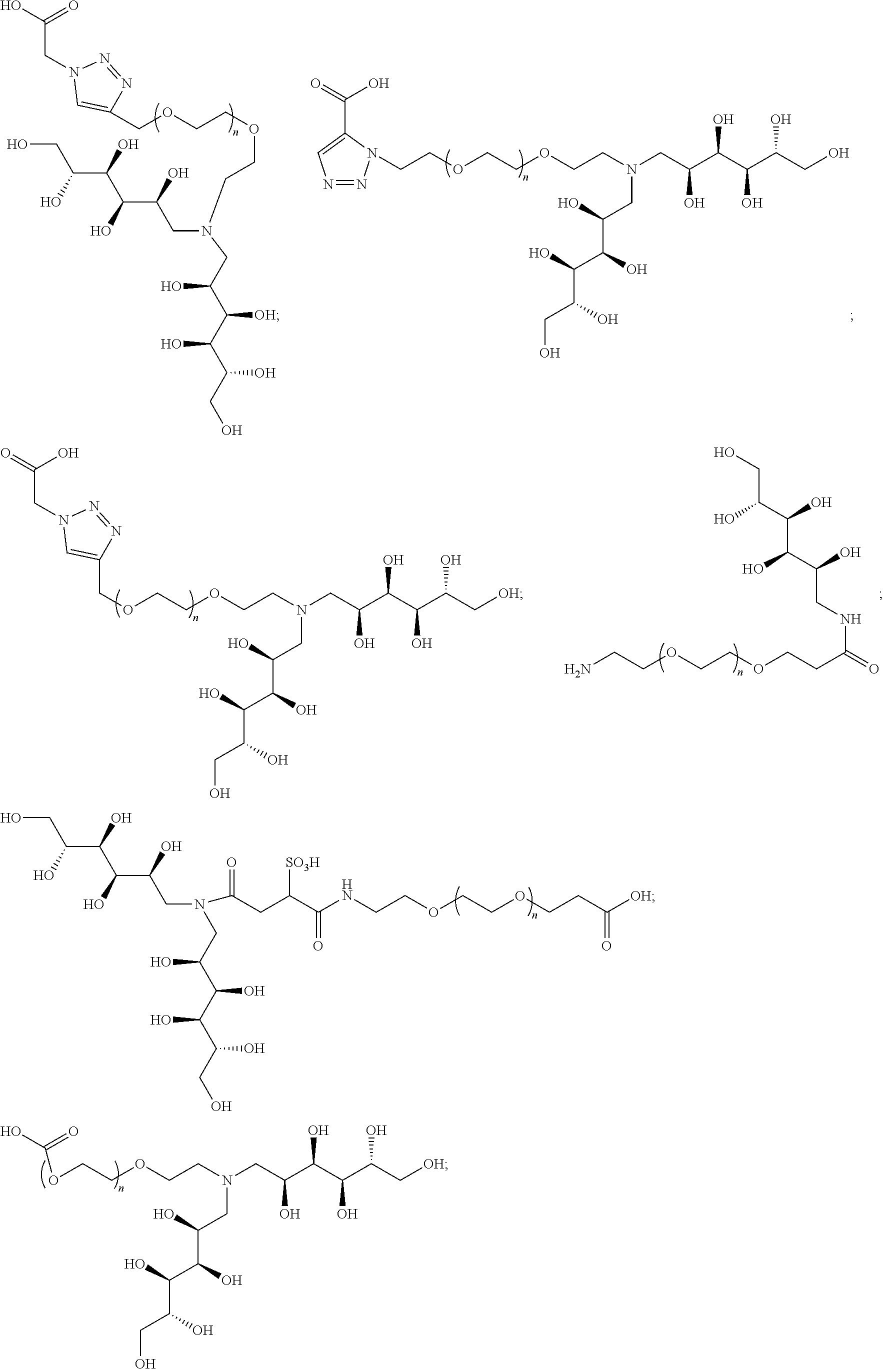 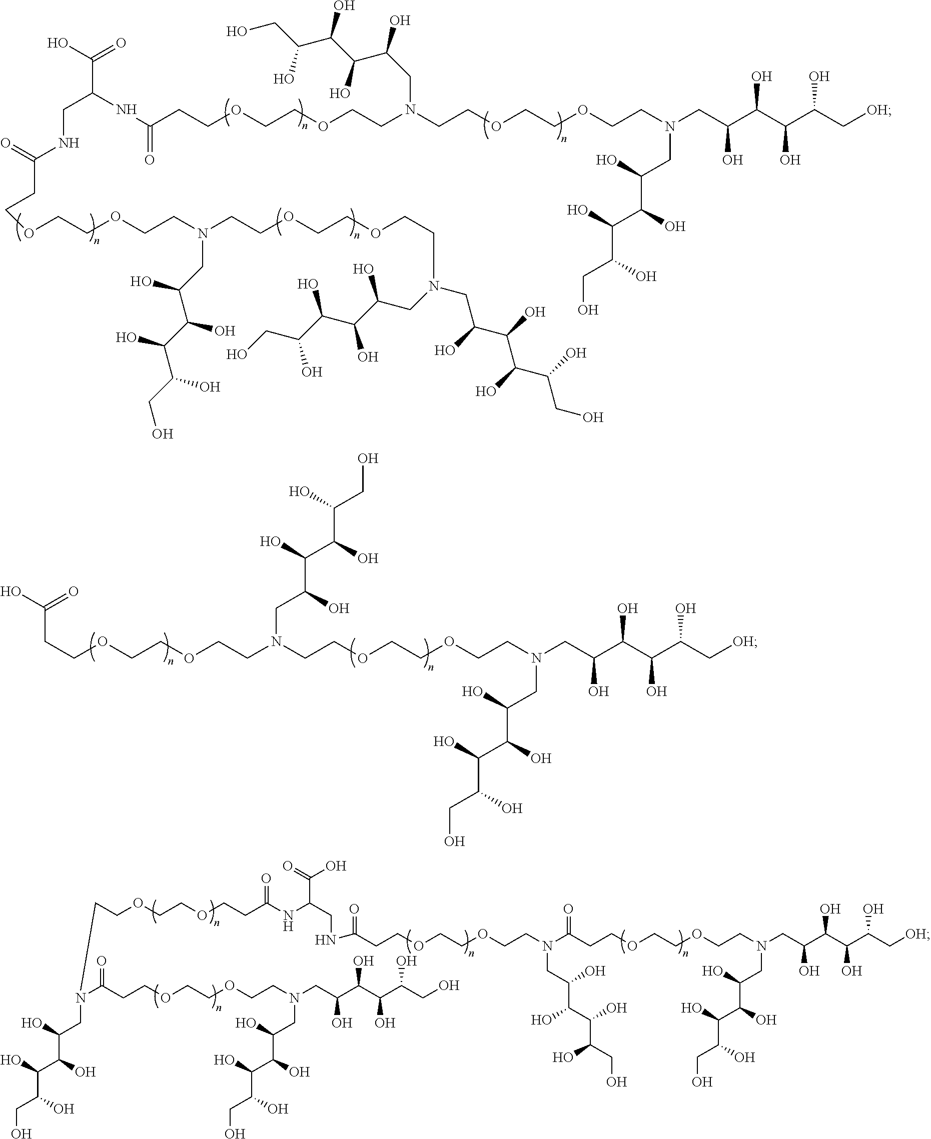 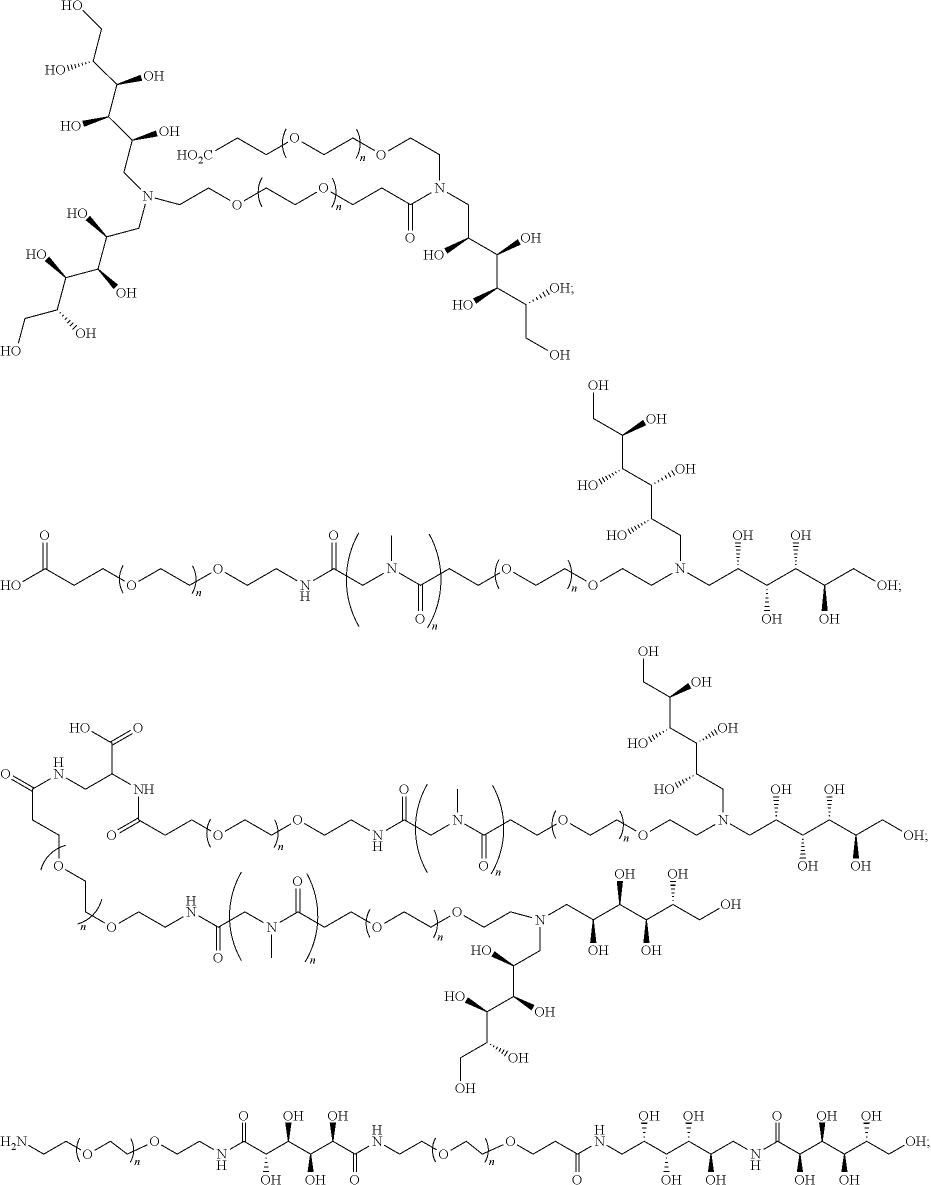 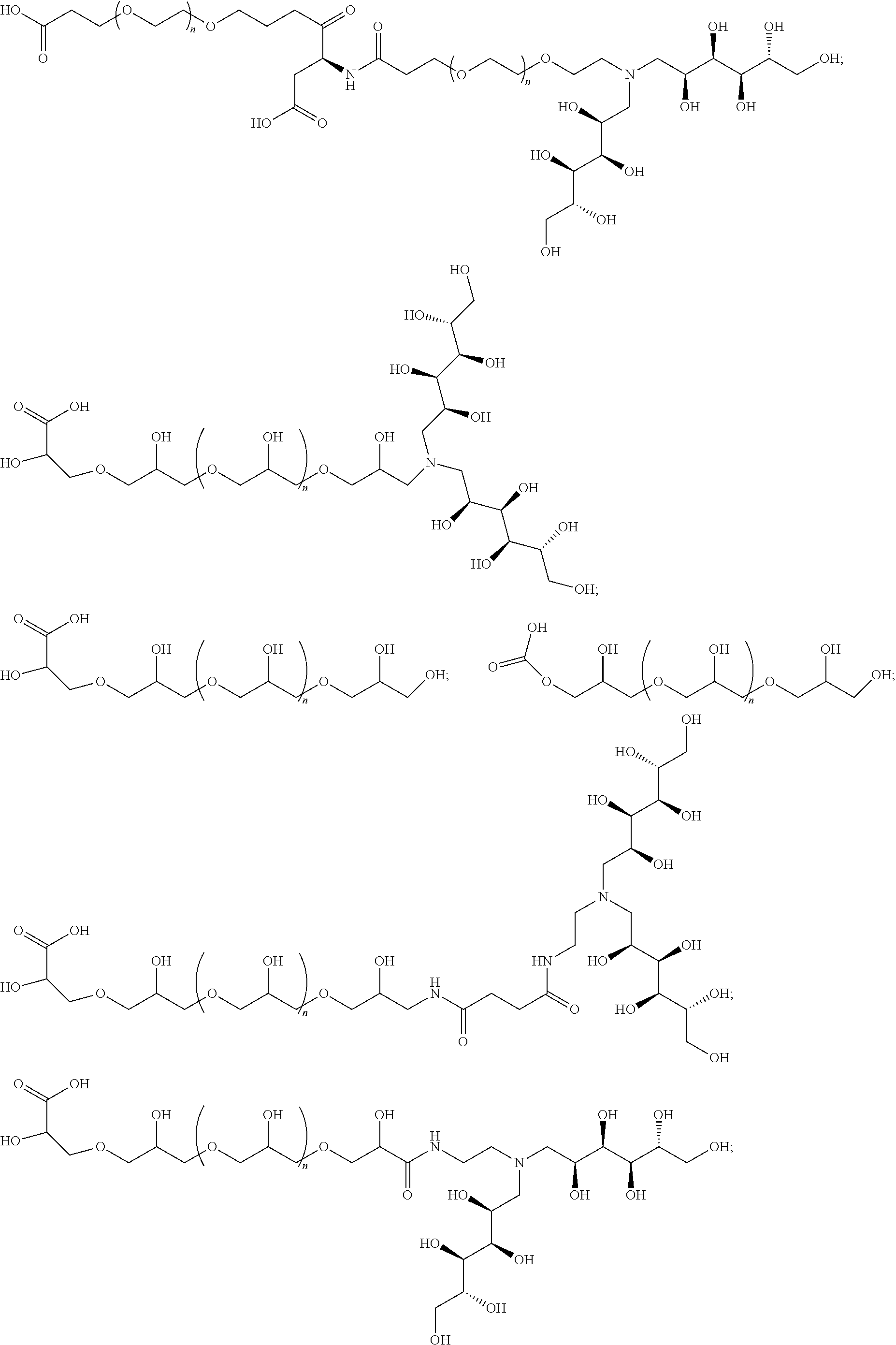 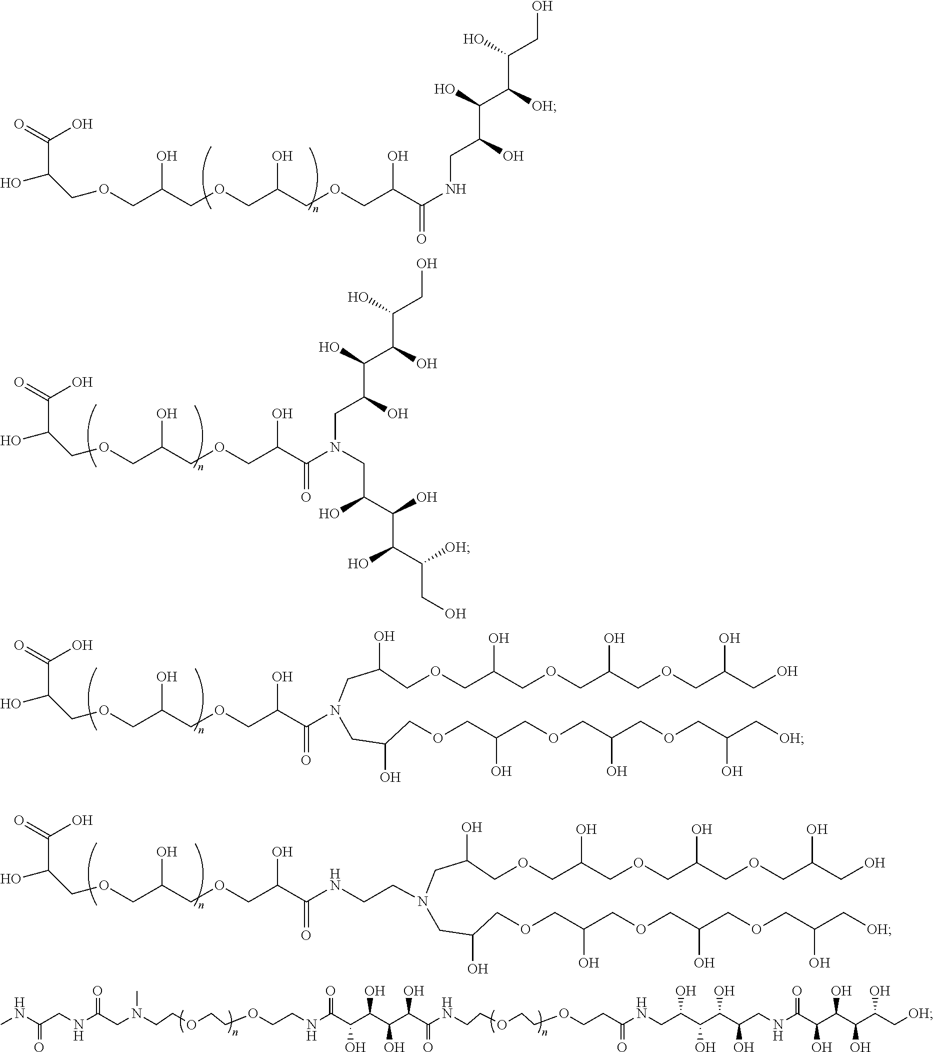 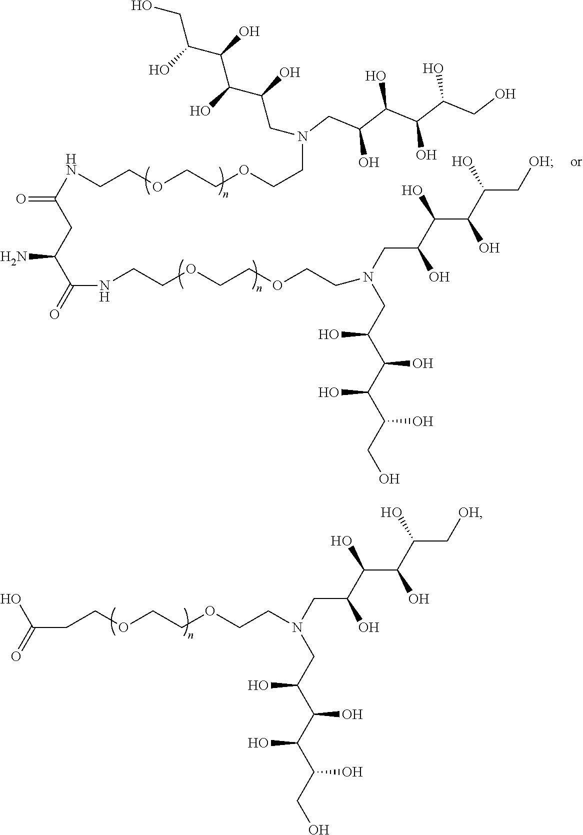
or a stereoisomer thereof, wherein R^{a} is H or C₁₋₆ alkyl and n is 1-20.

68. The Linker compound of any one of claims 1-67, comprising a Polar group selected from the following: 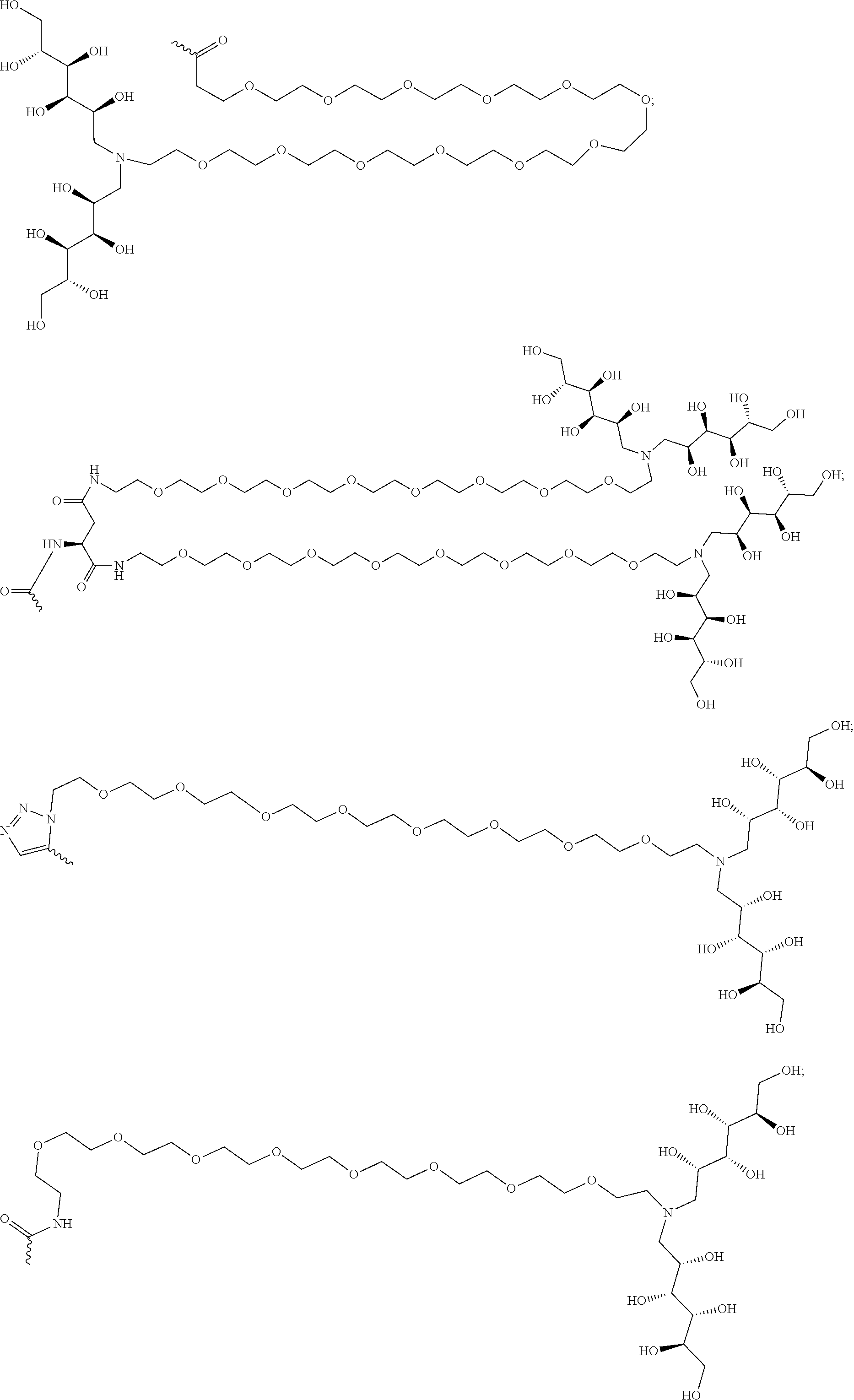 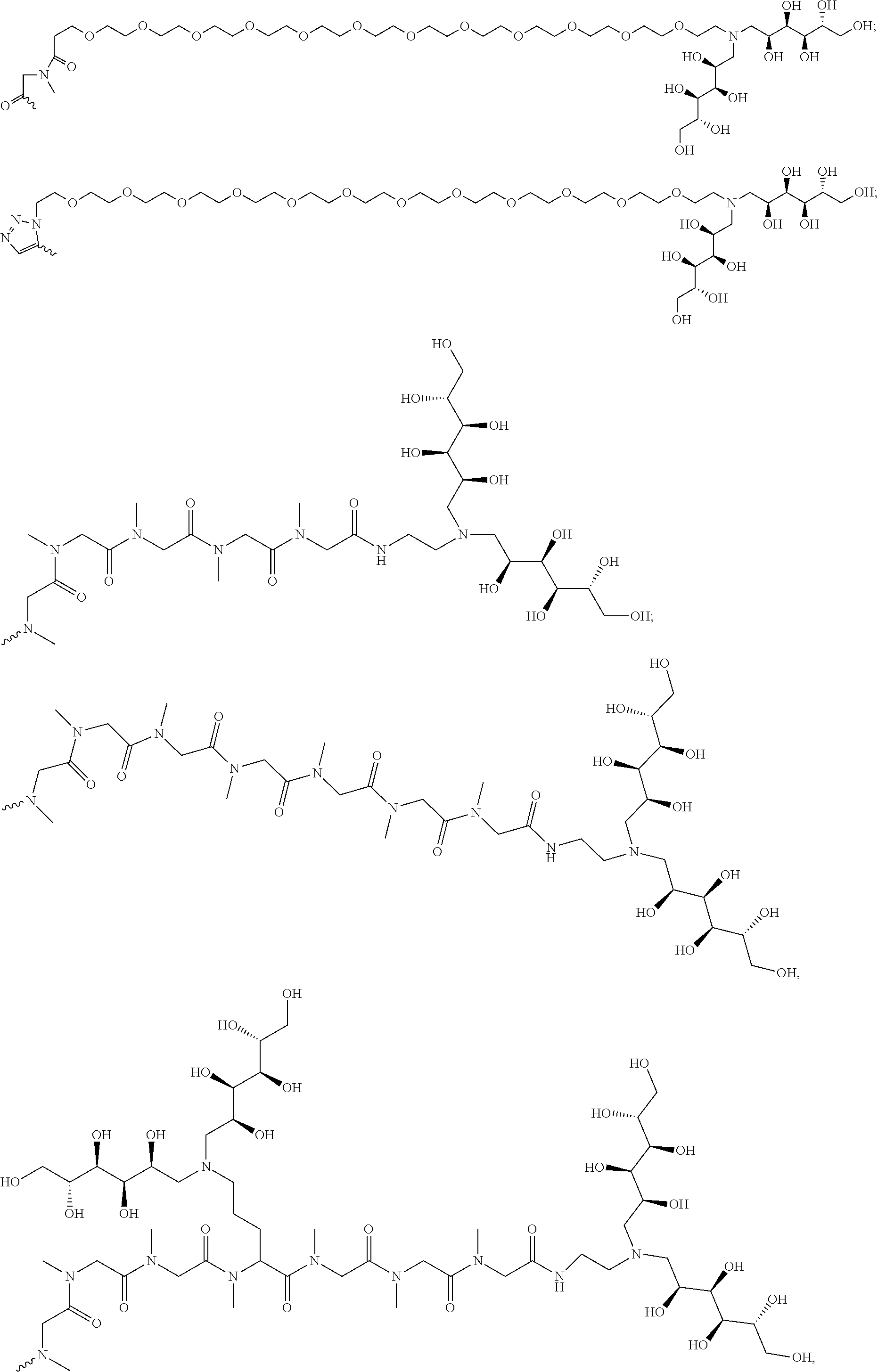 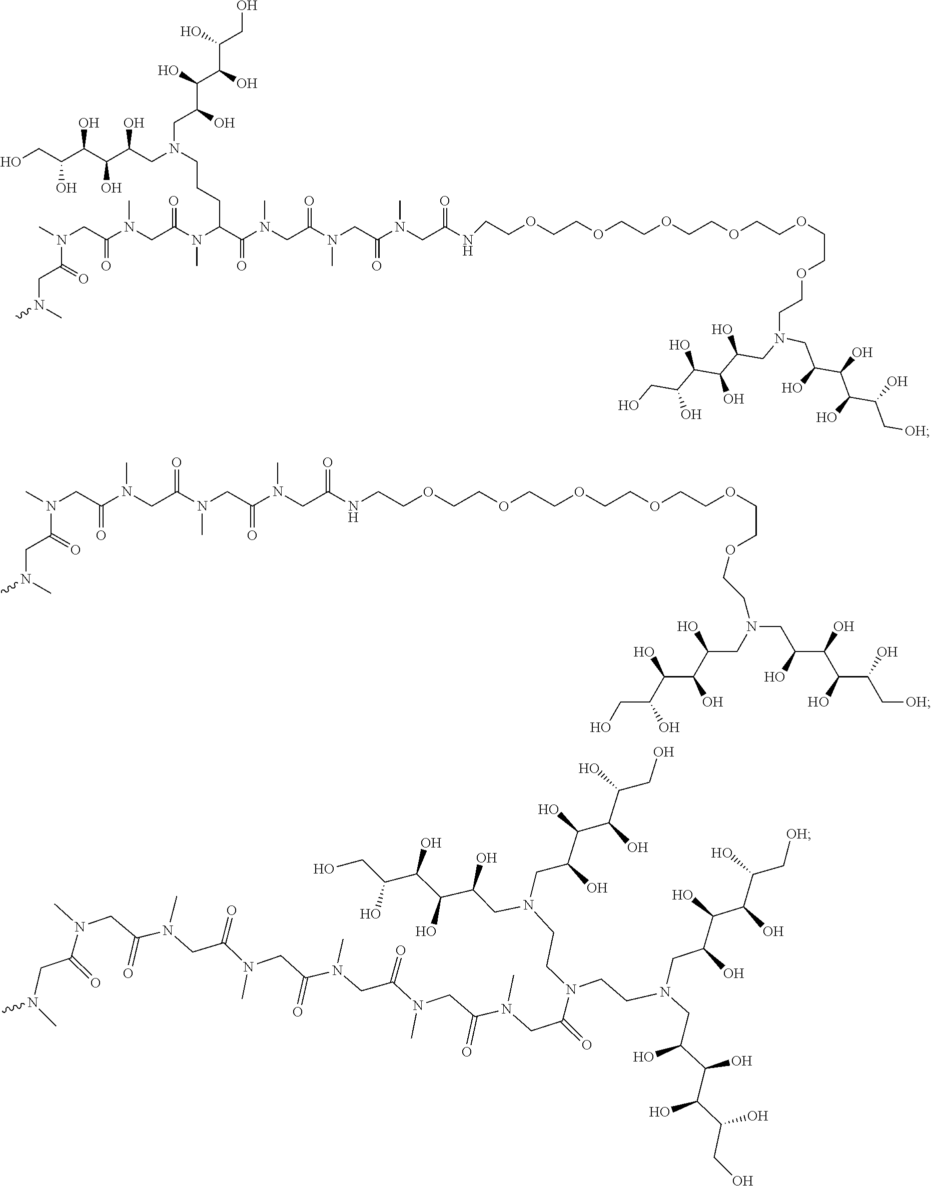 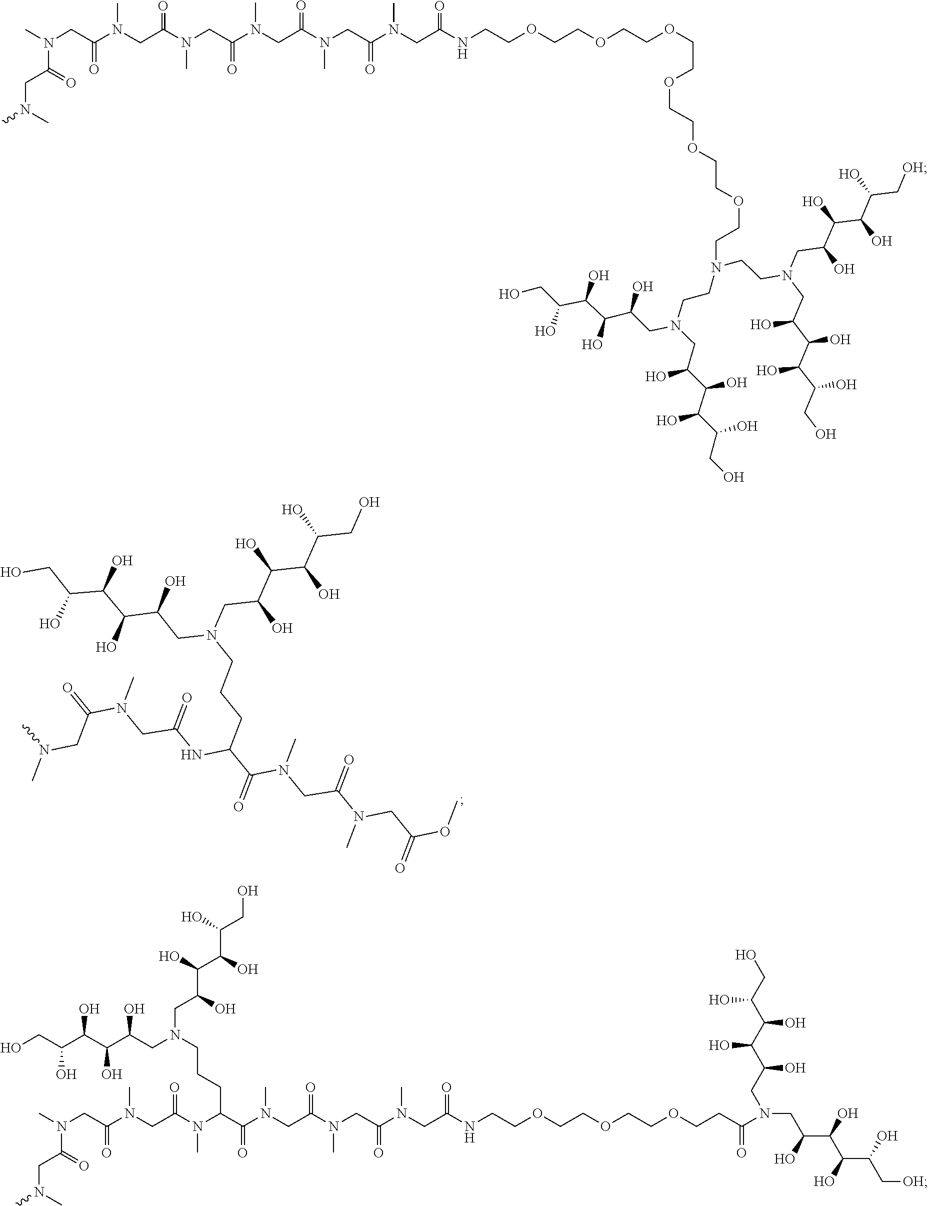 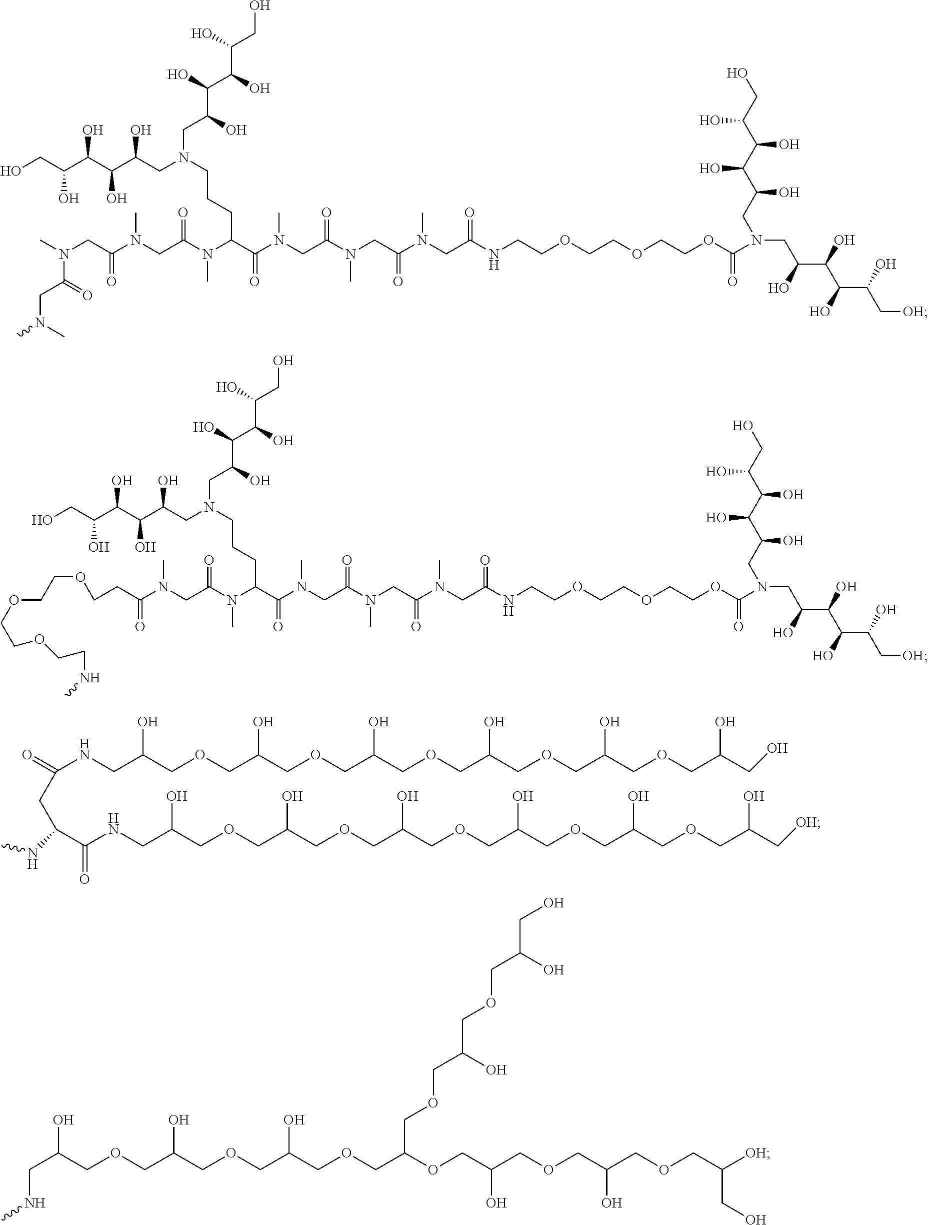 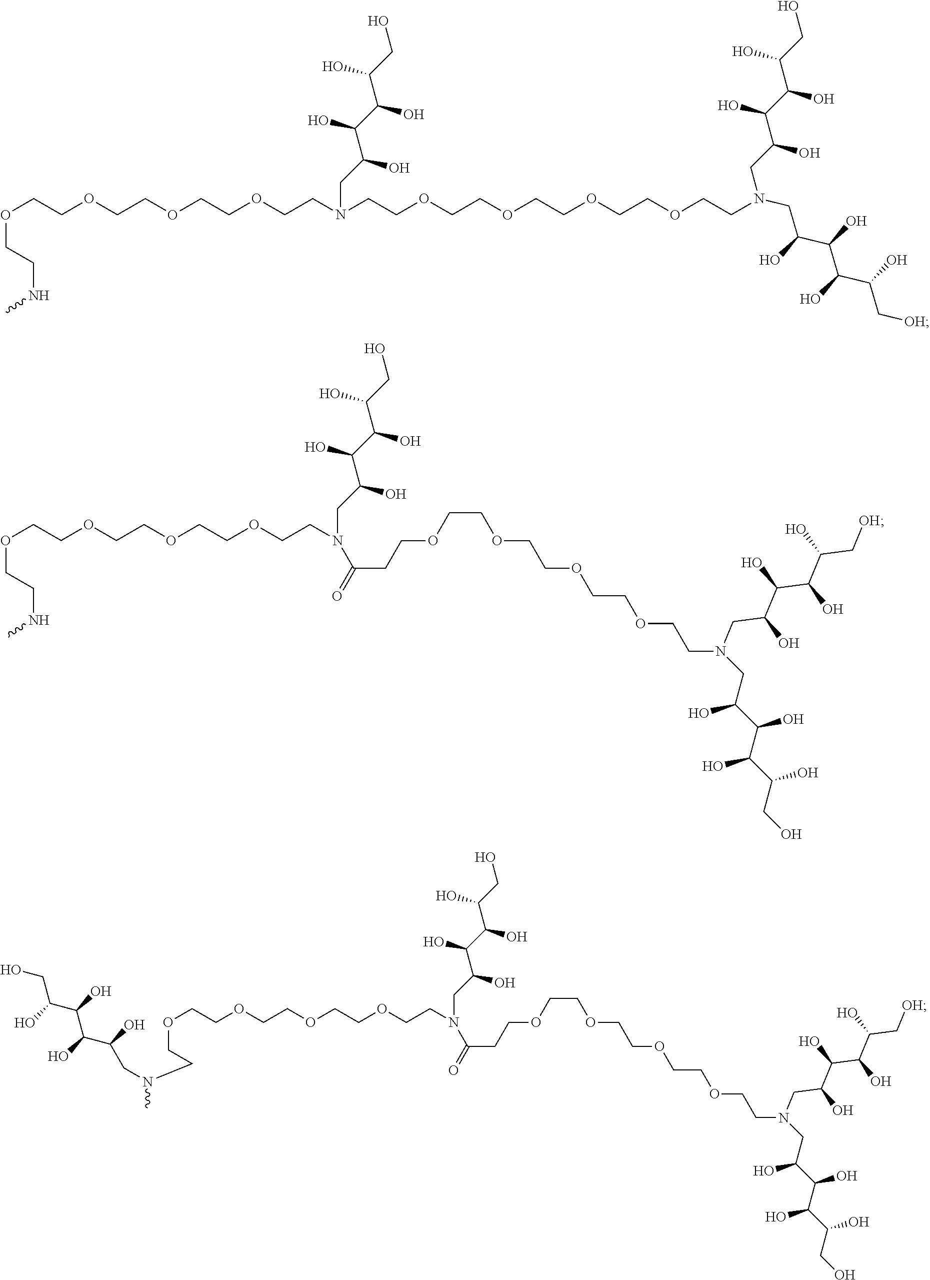 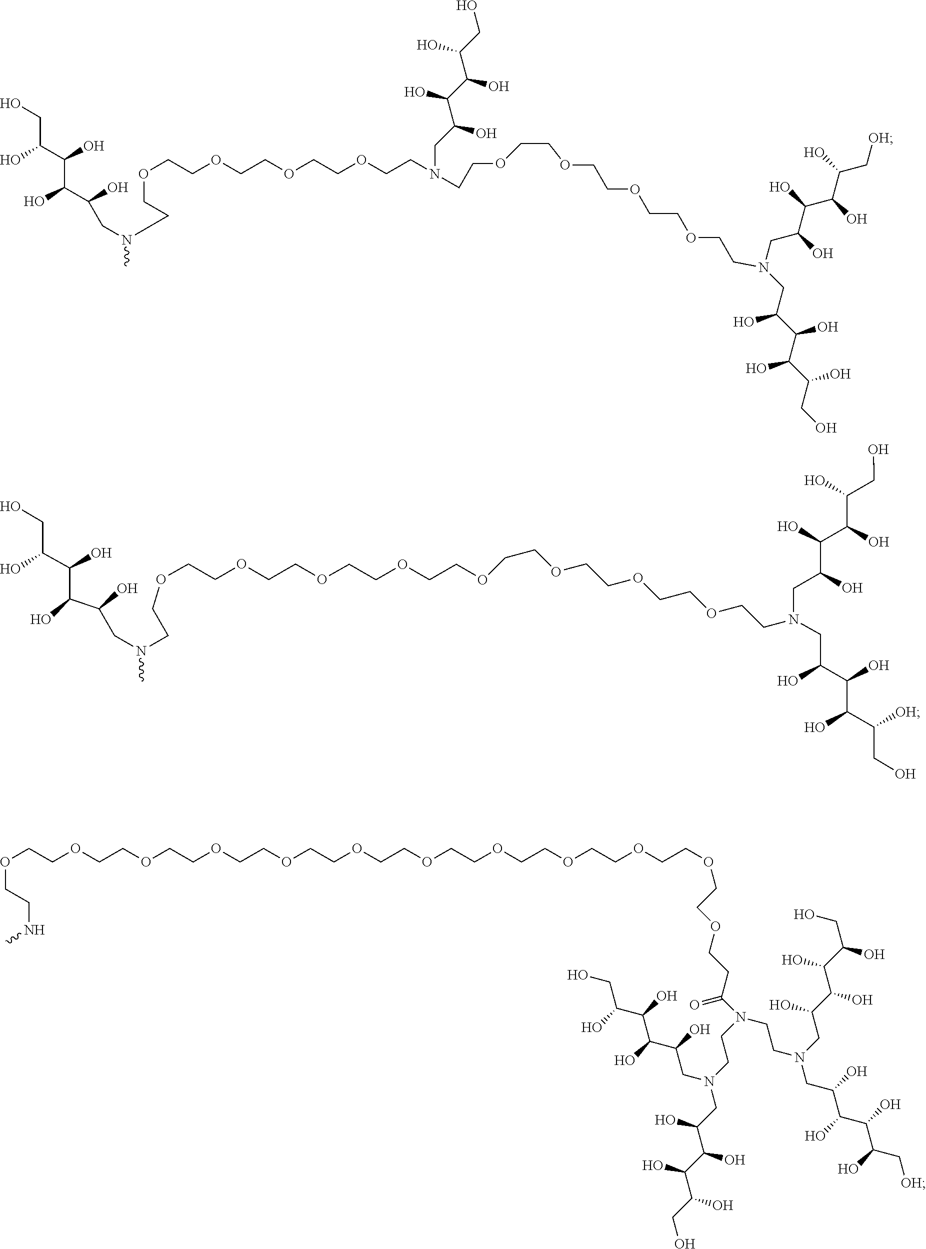 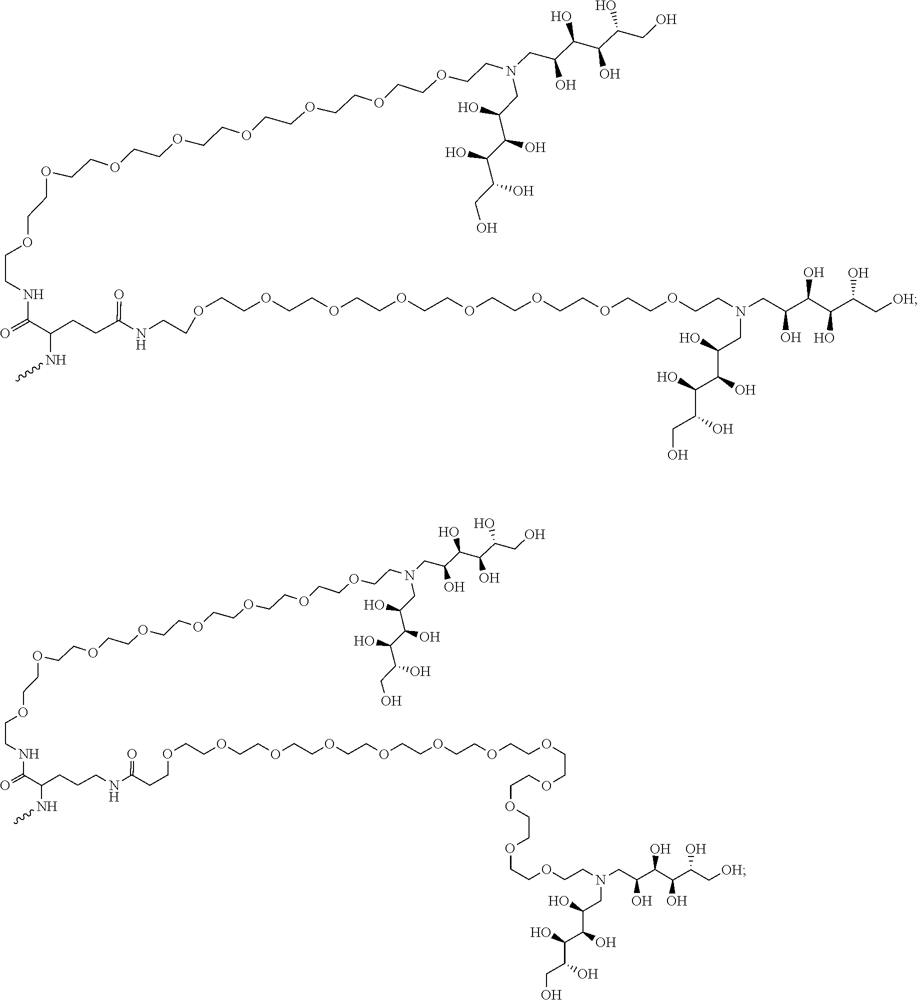 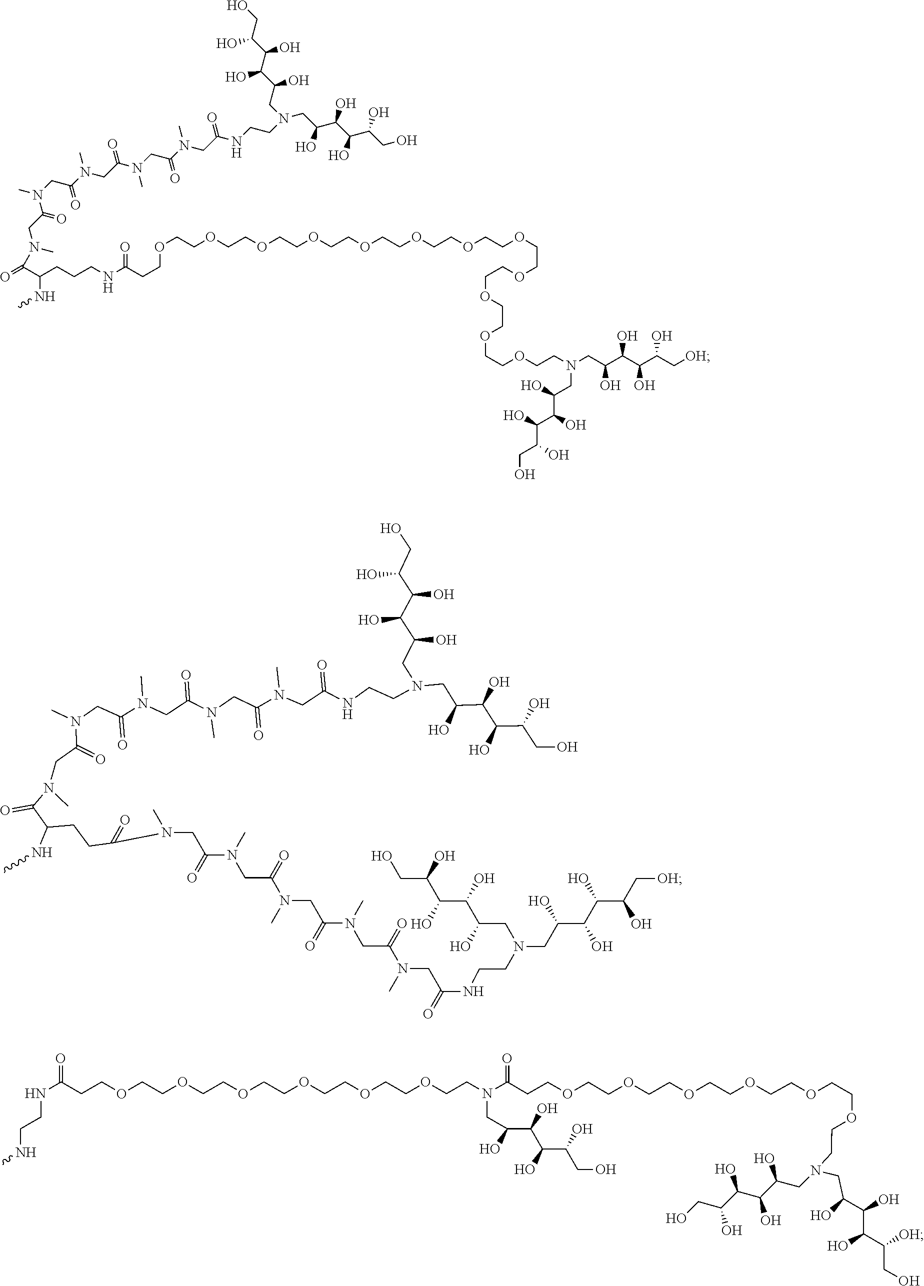 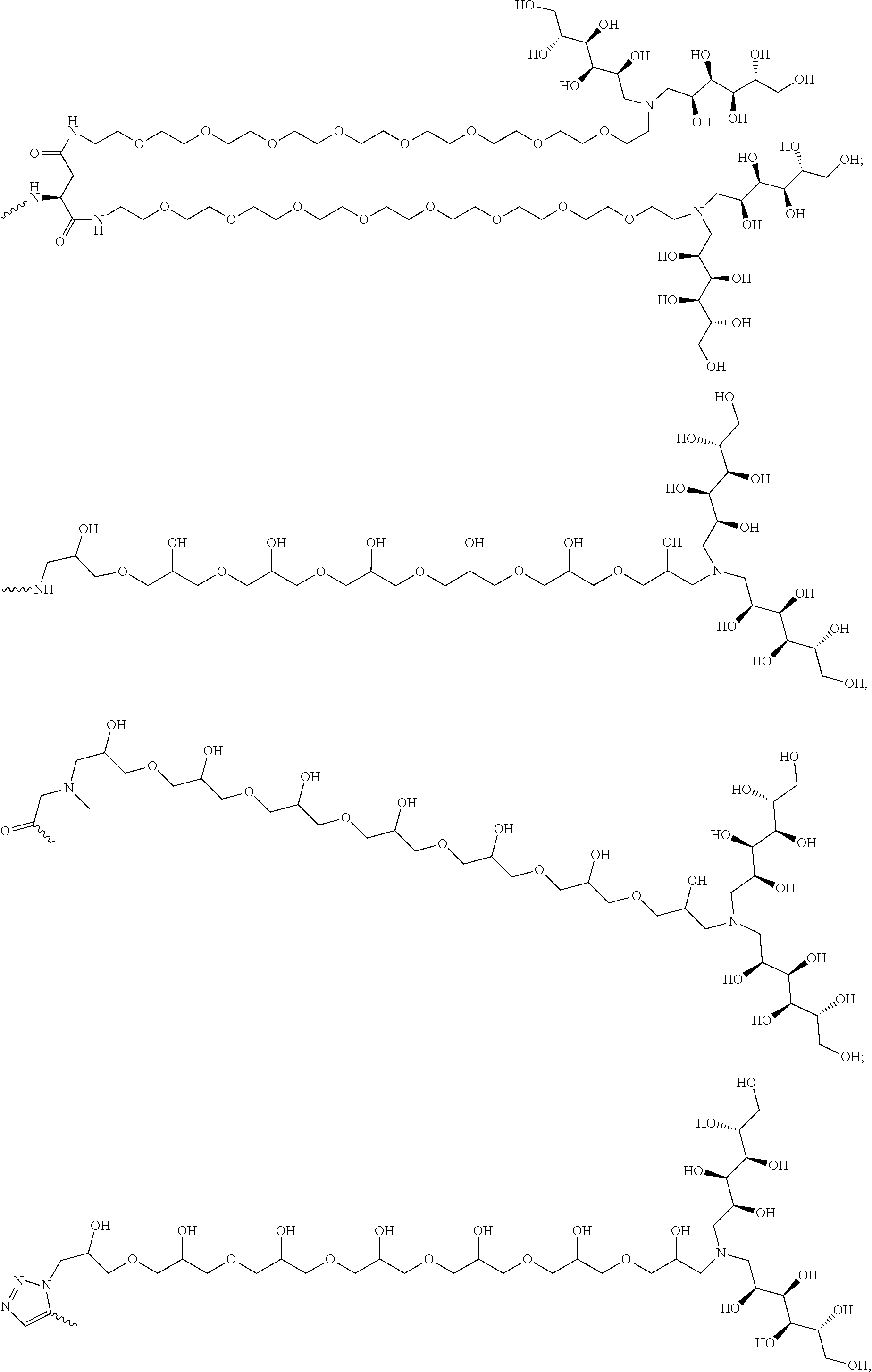 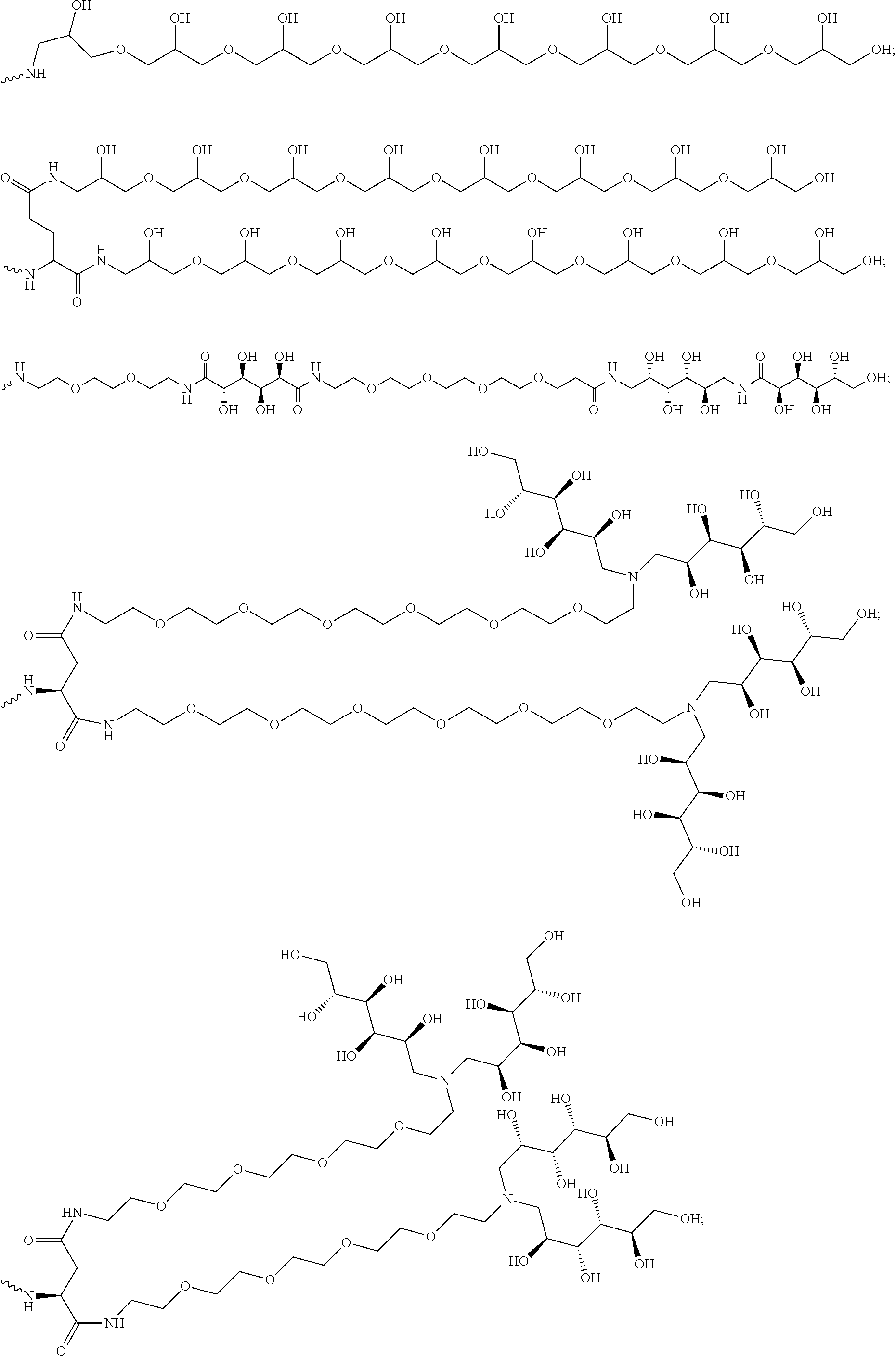 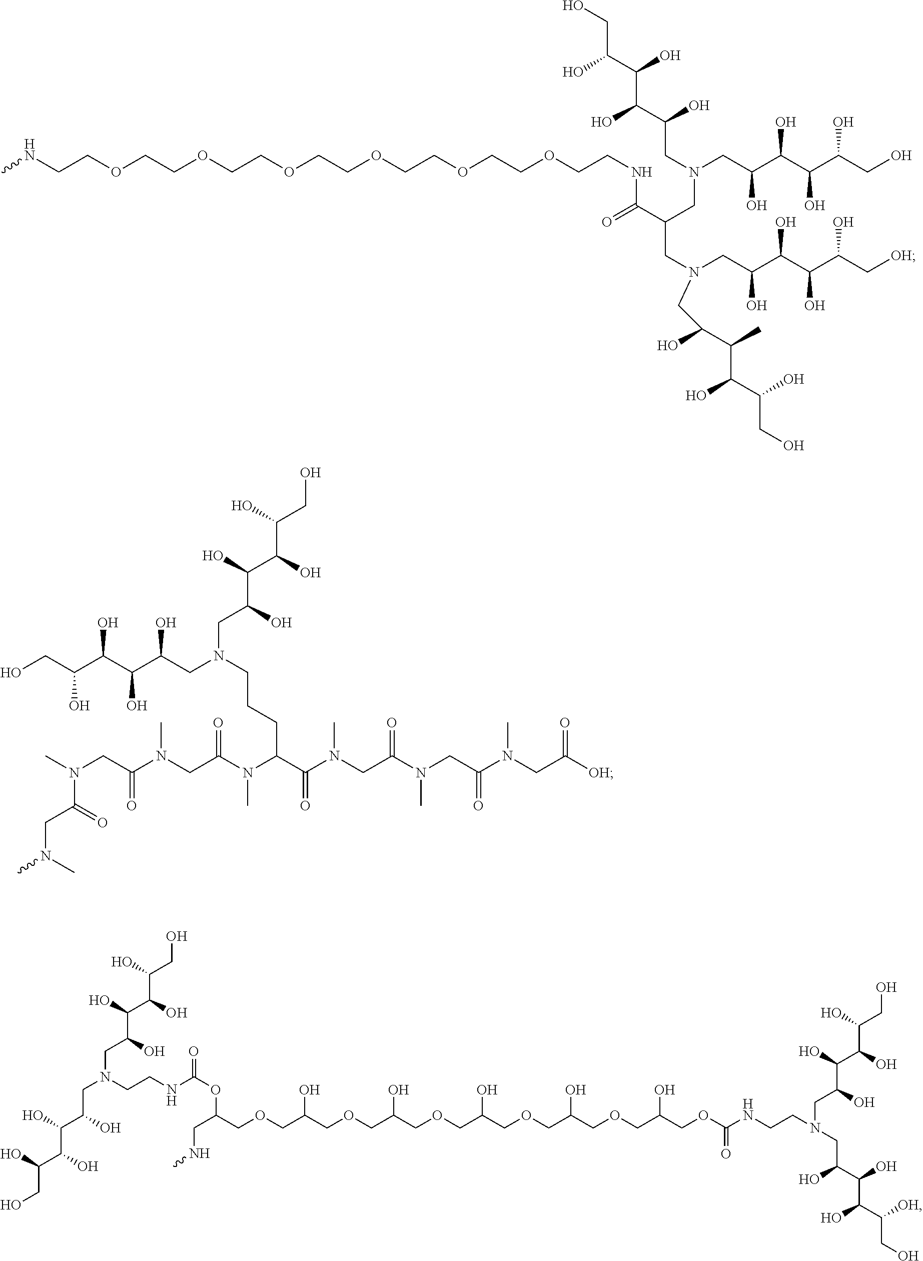 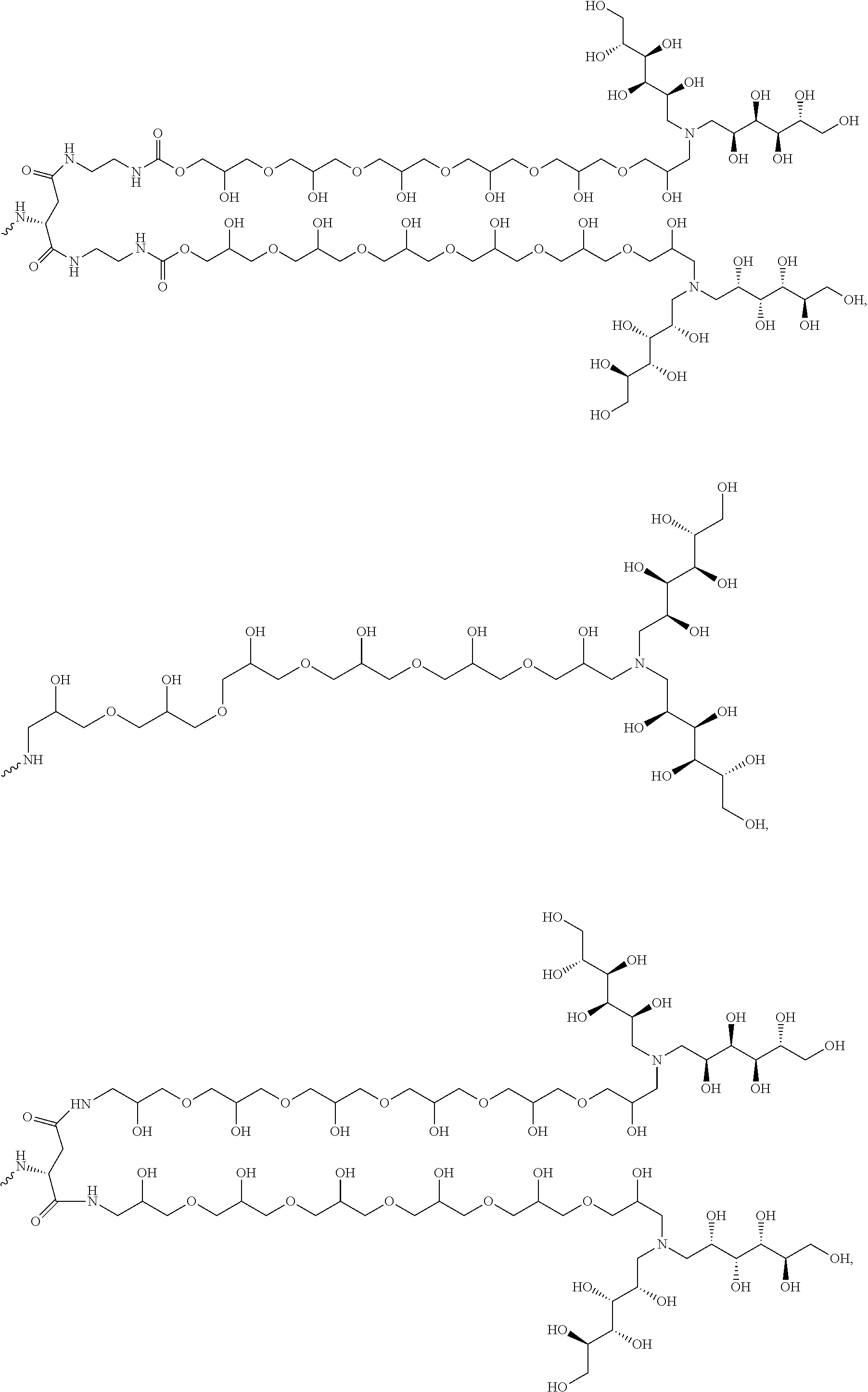 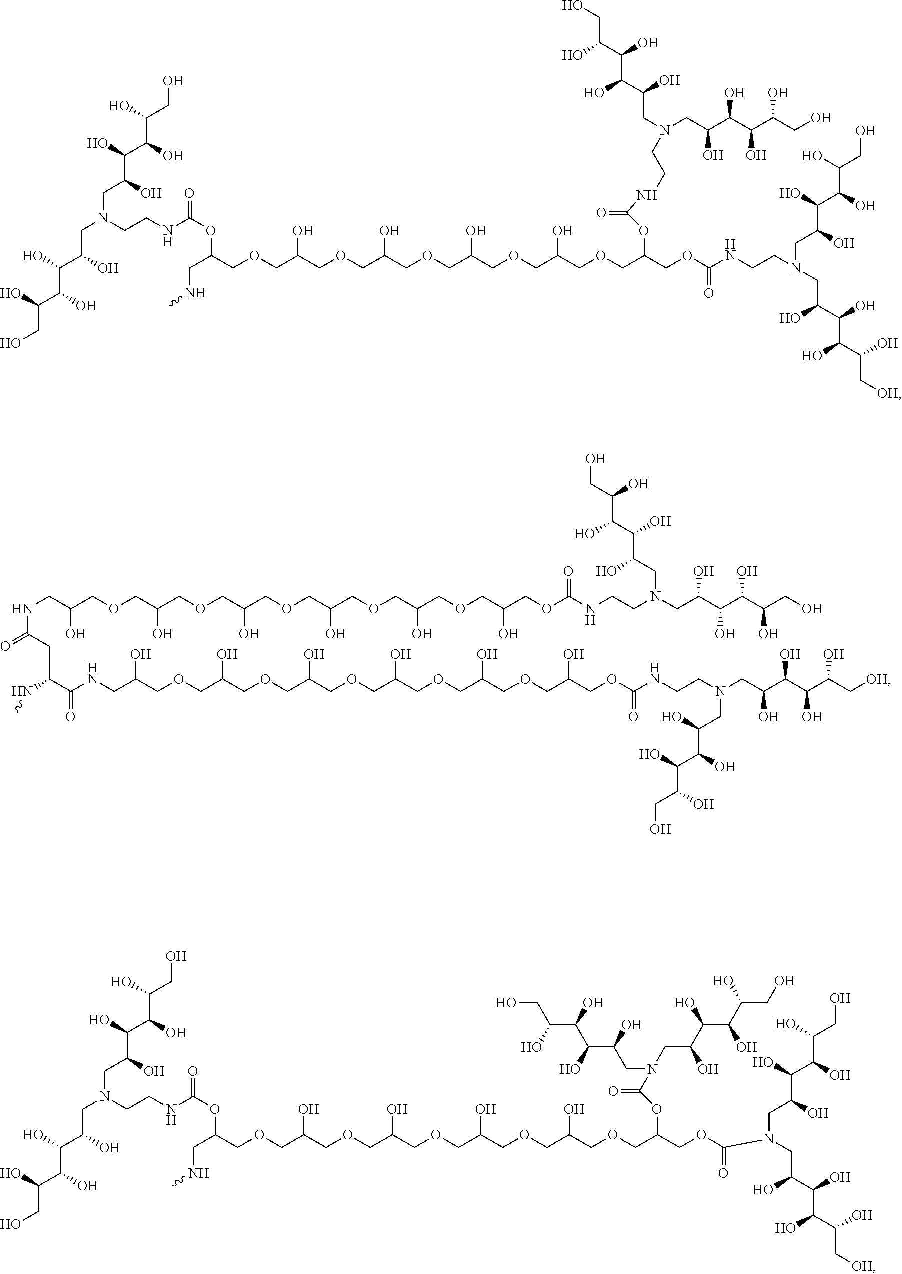 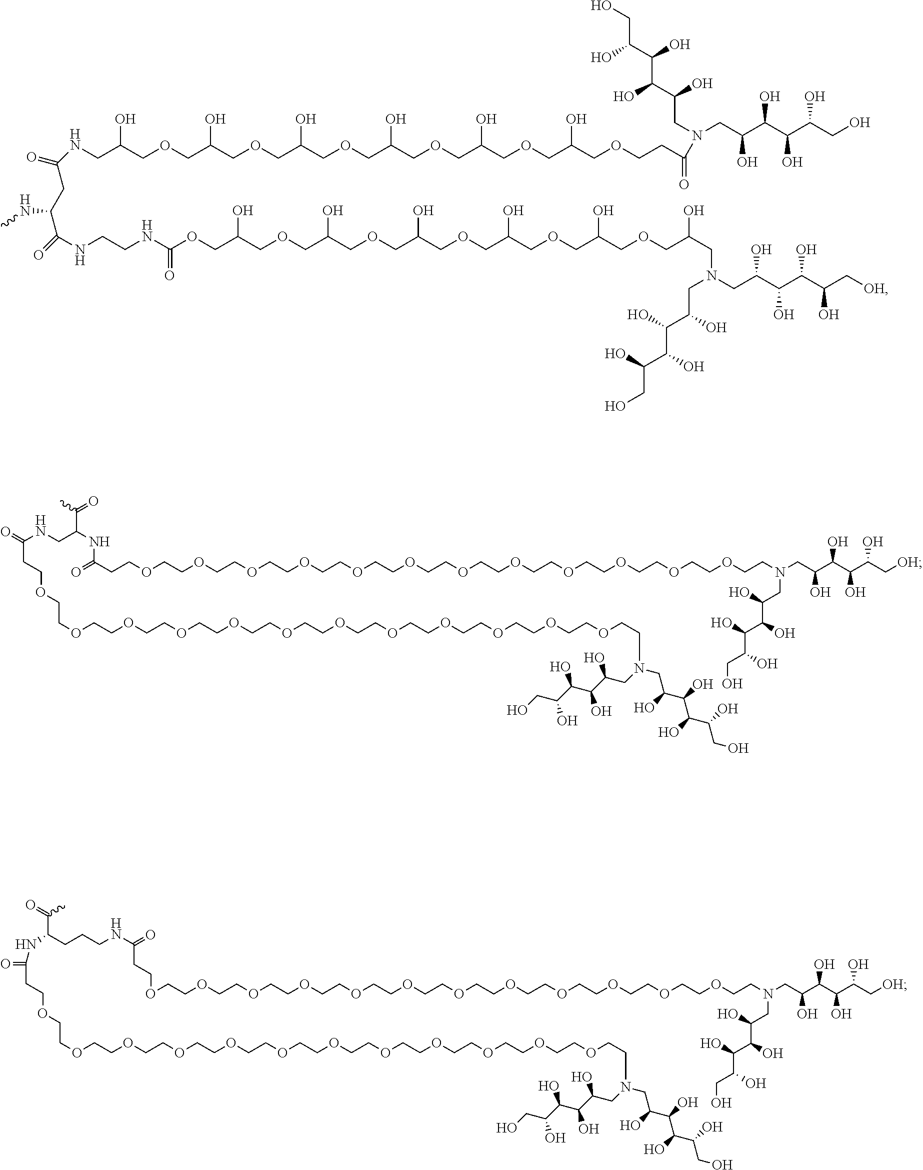 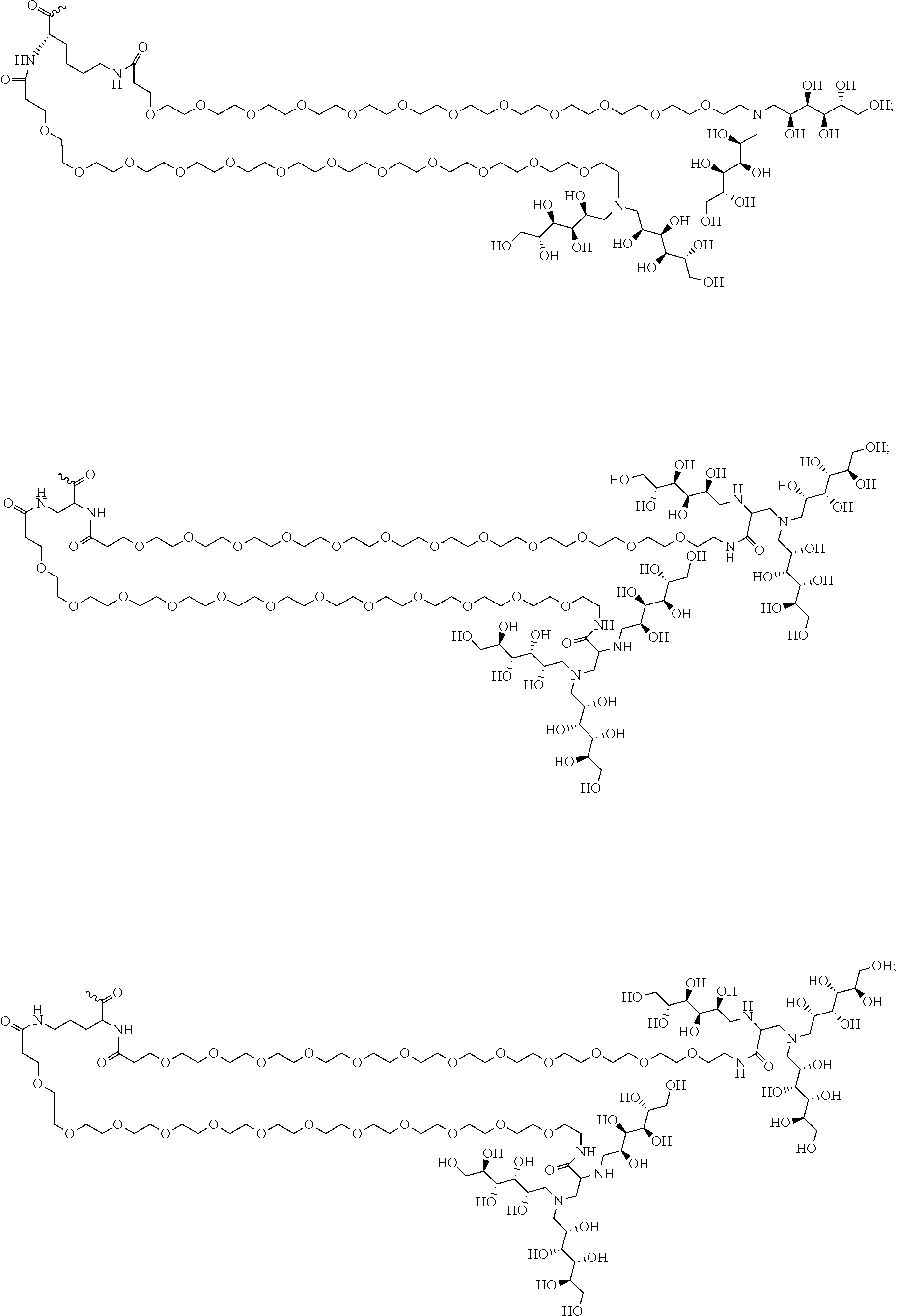 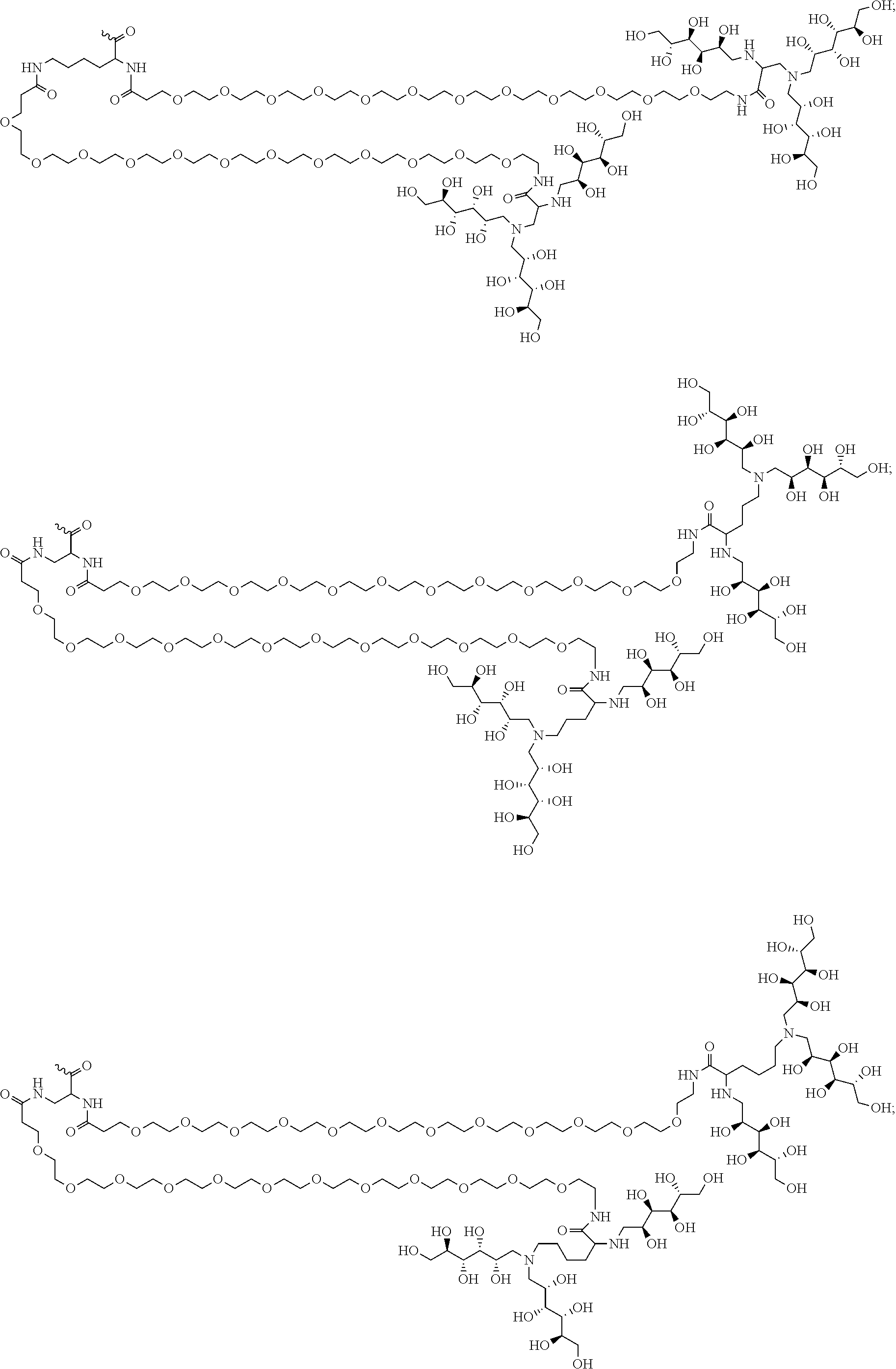 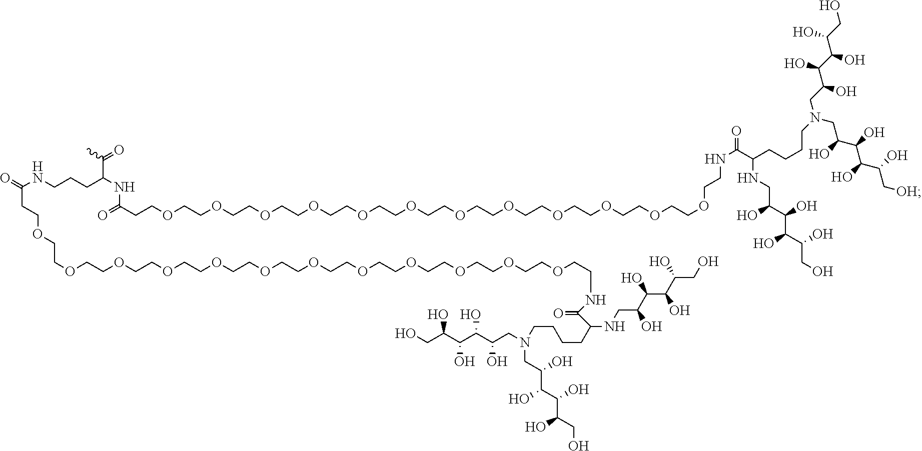 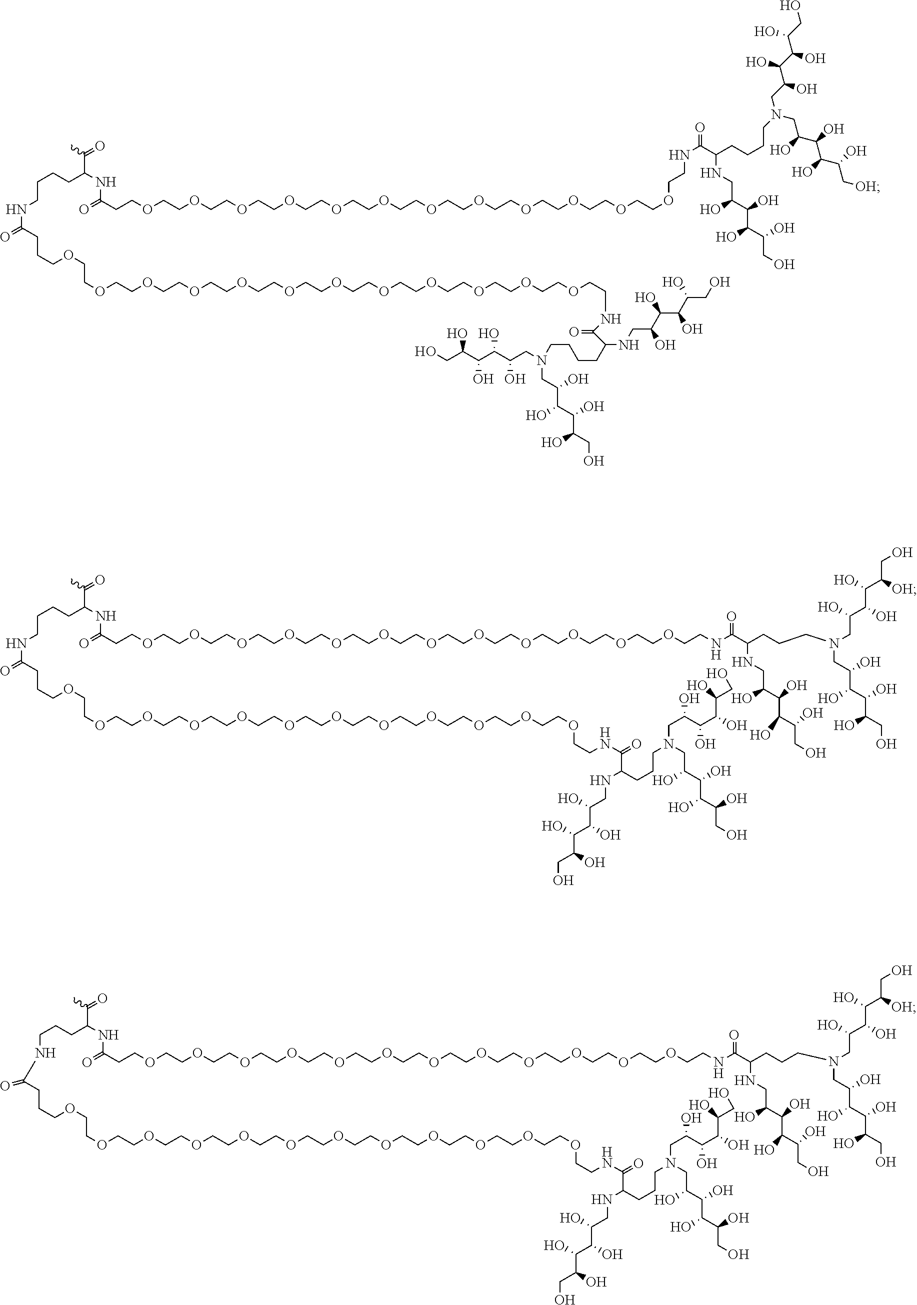 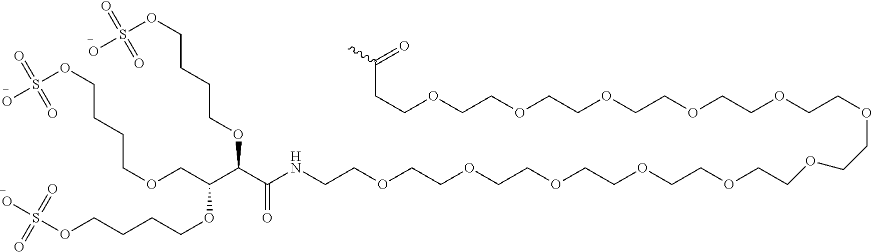 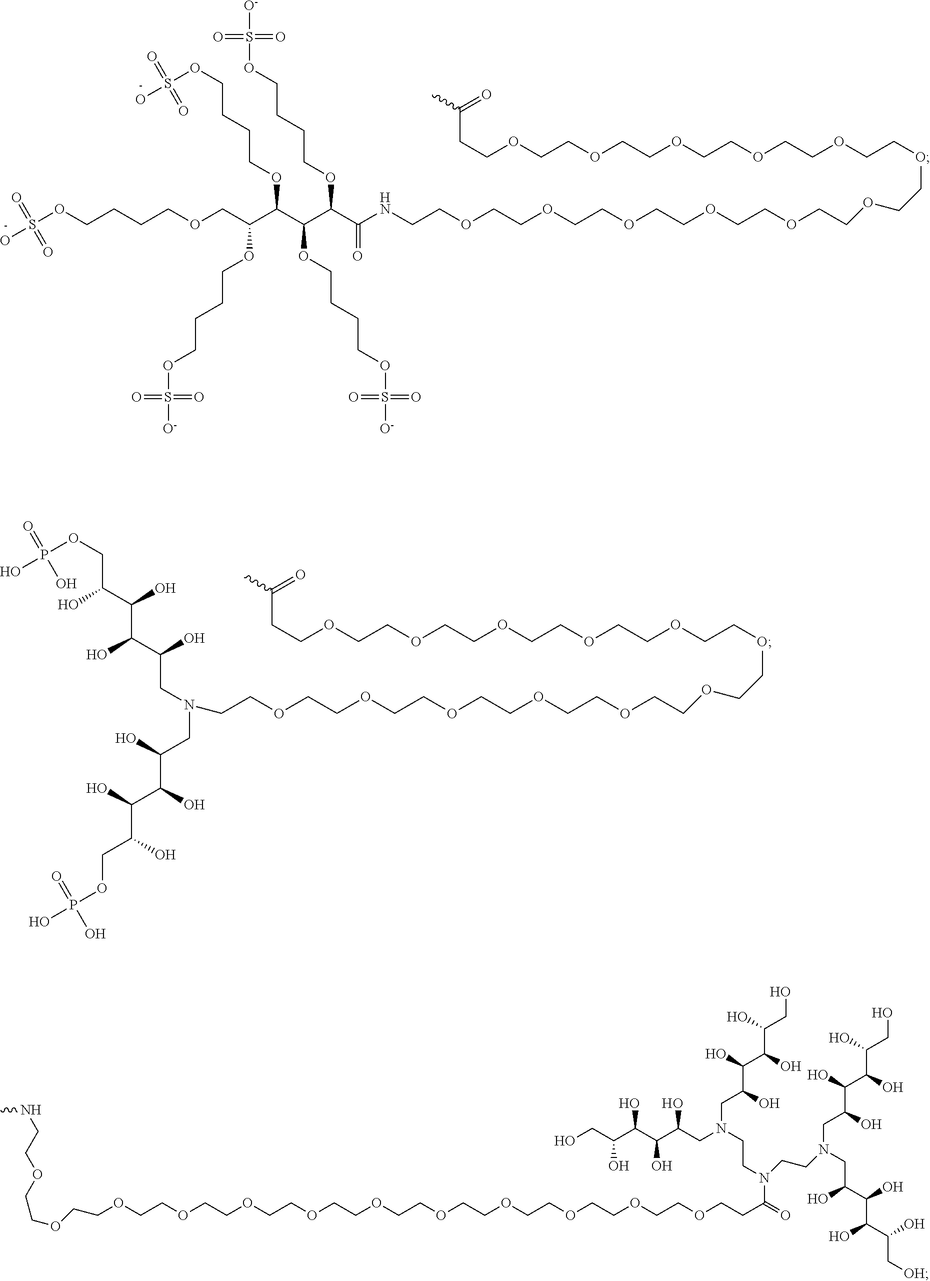 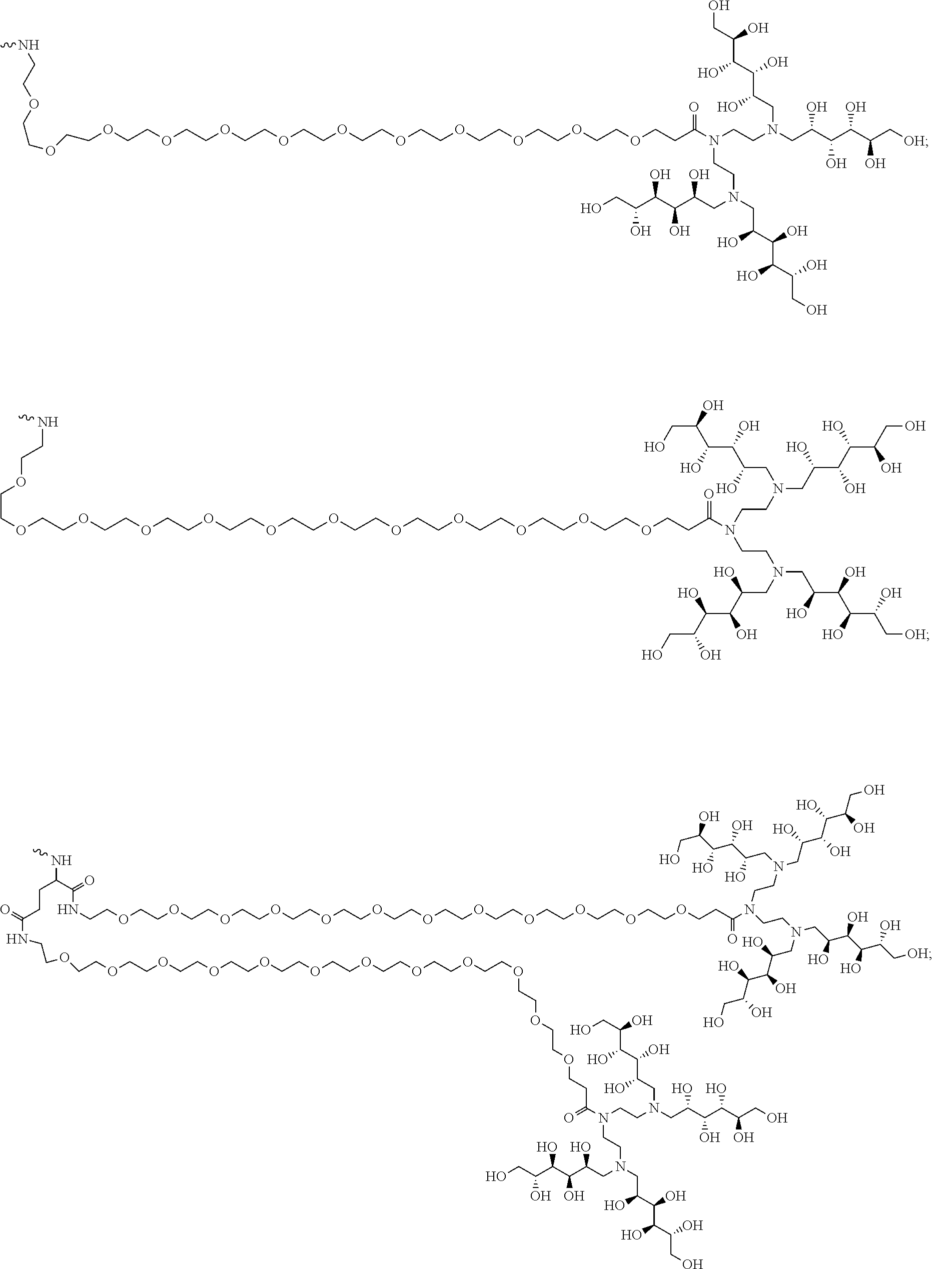 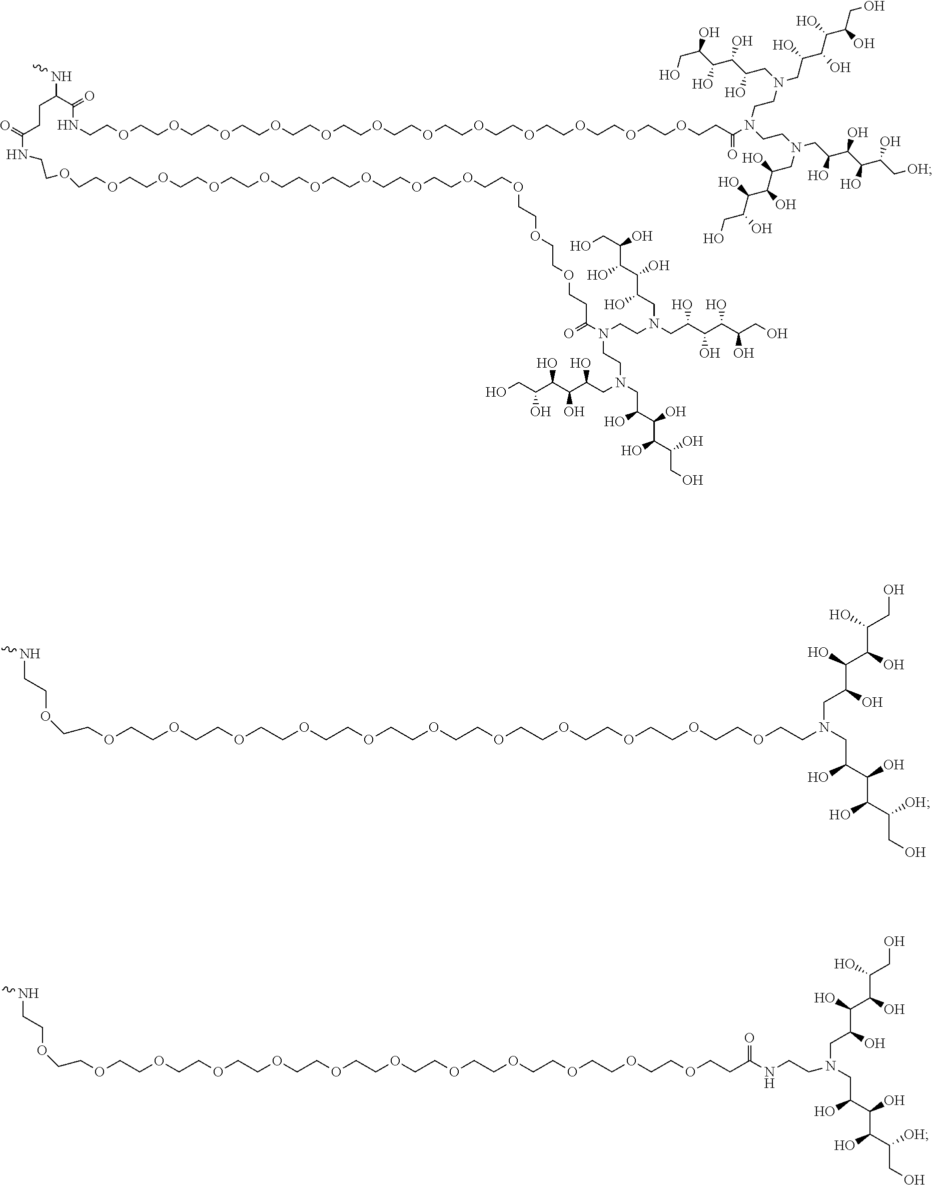 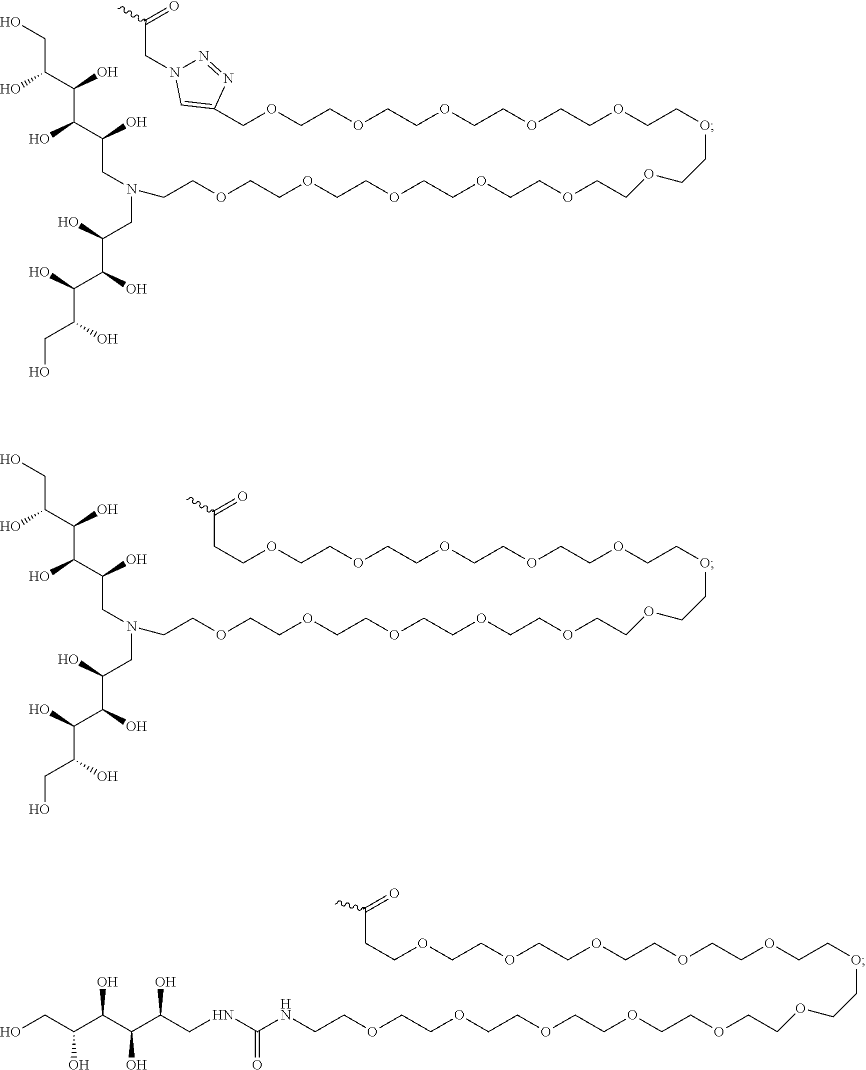 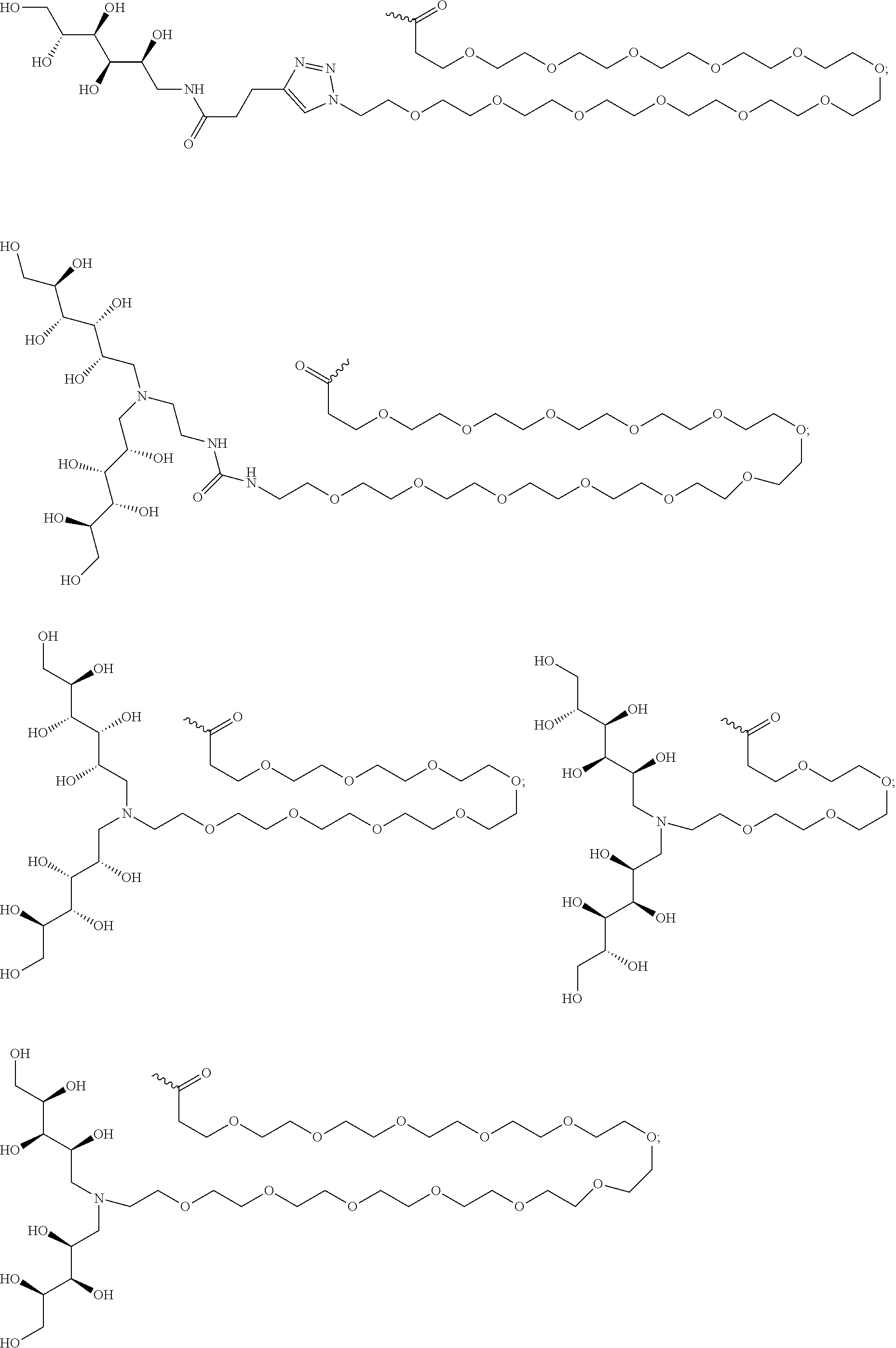 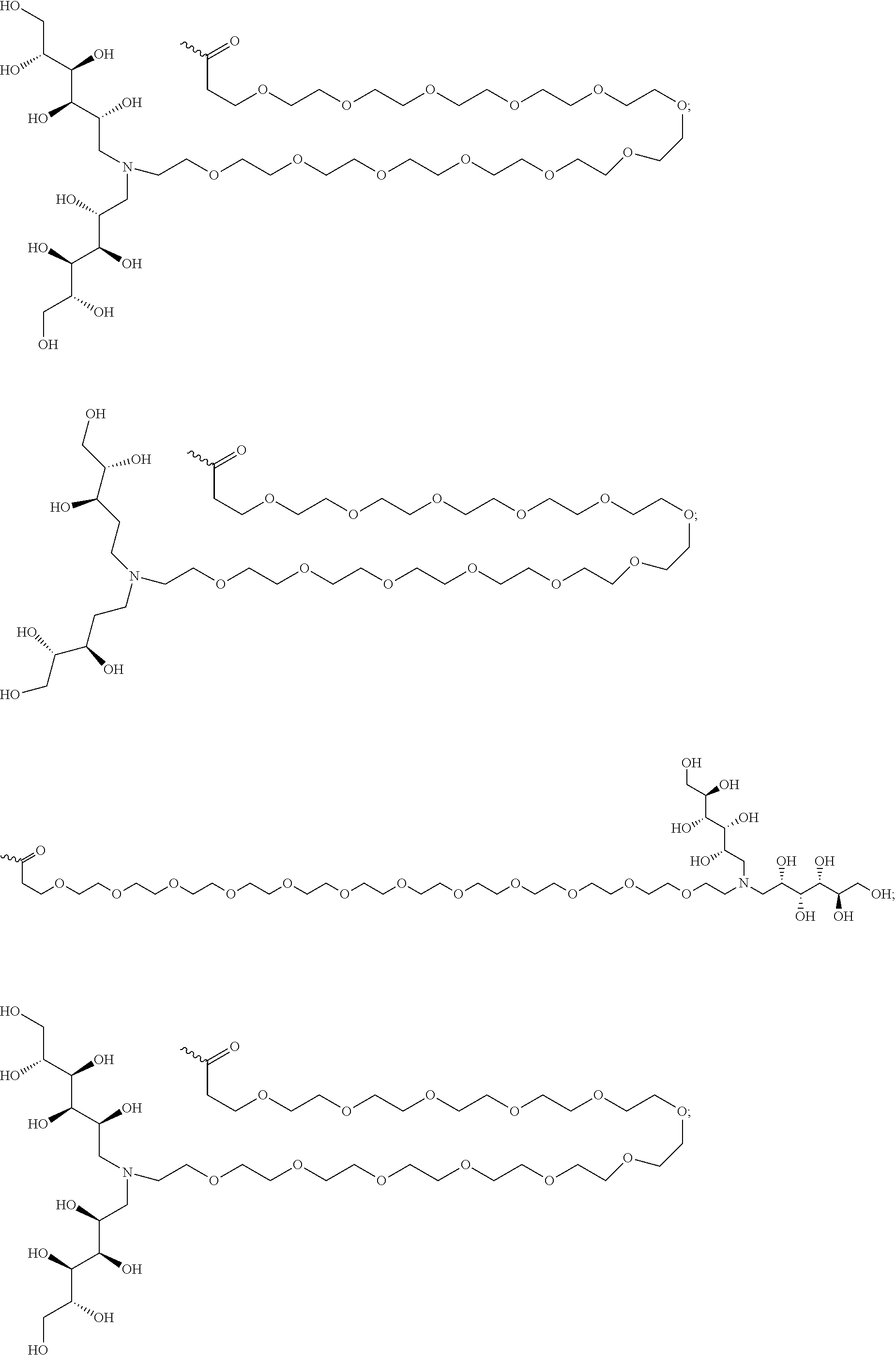 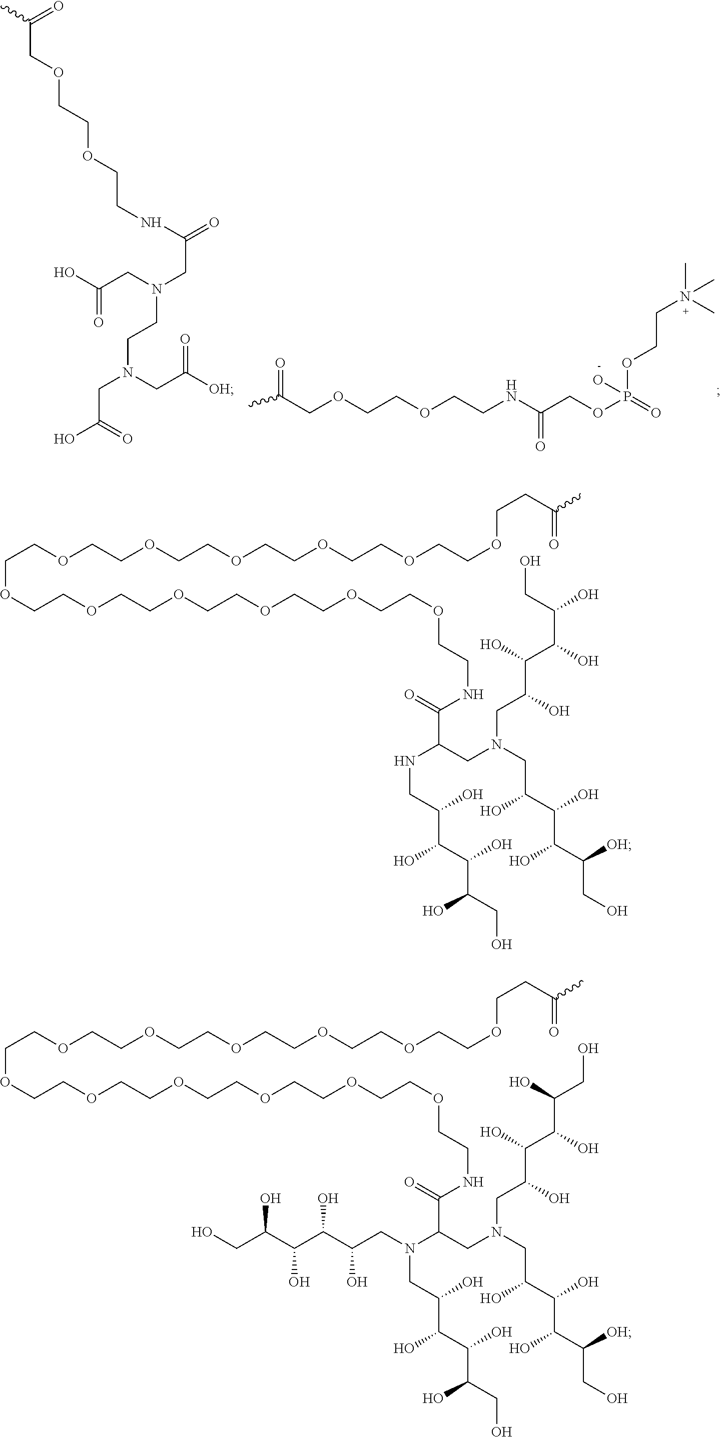 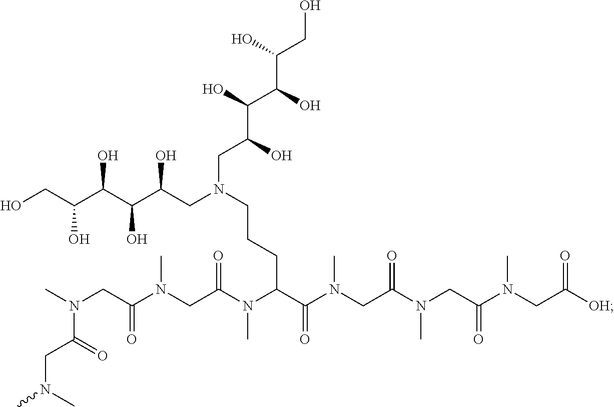 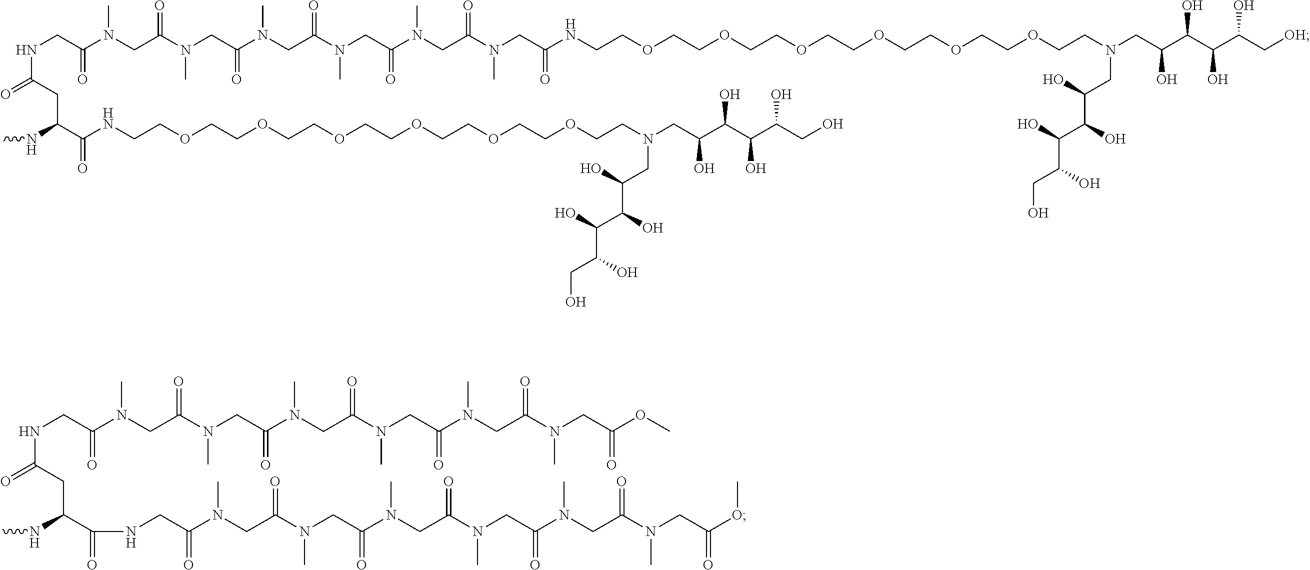 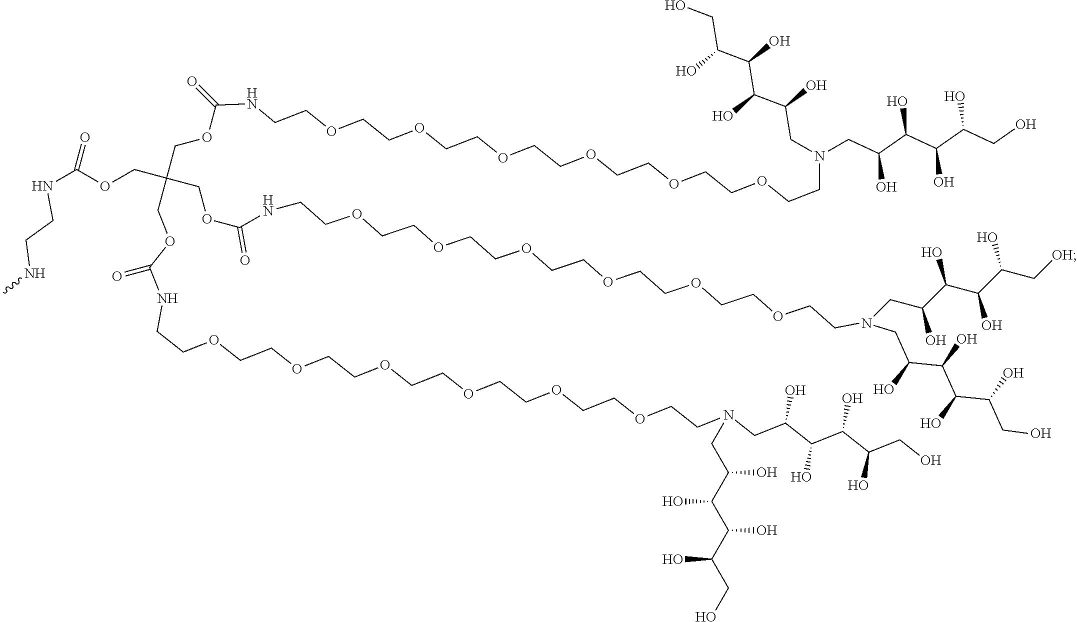 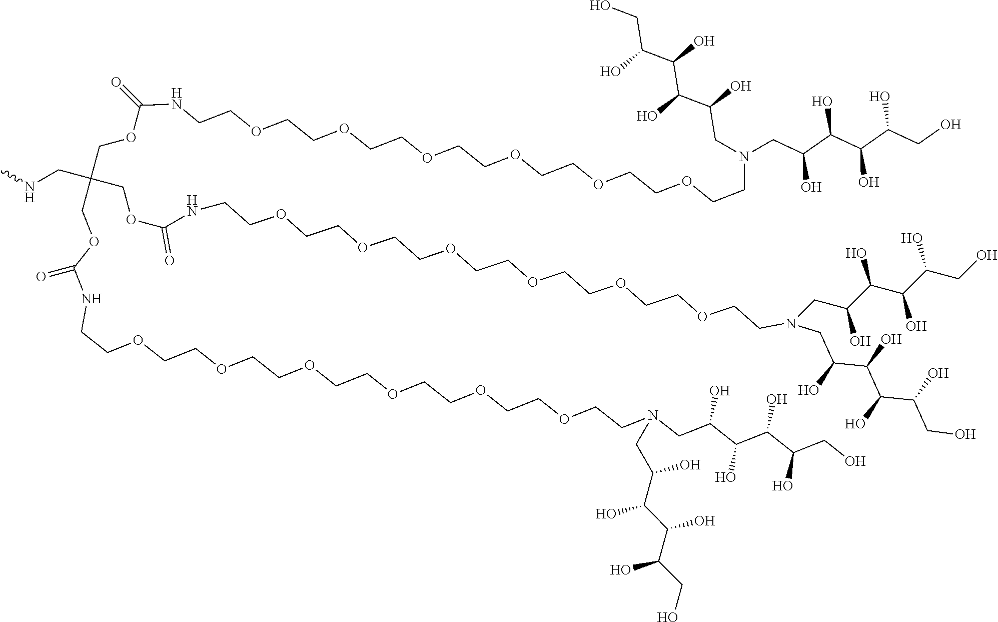 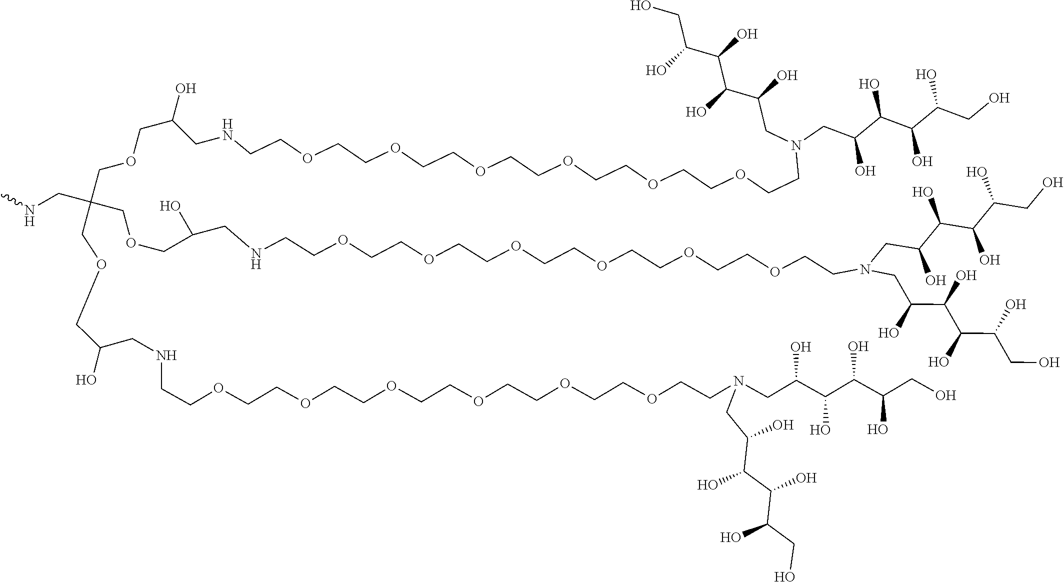 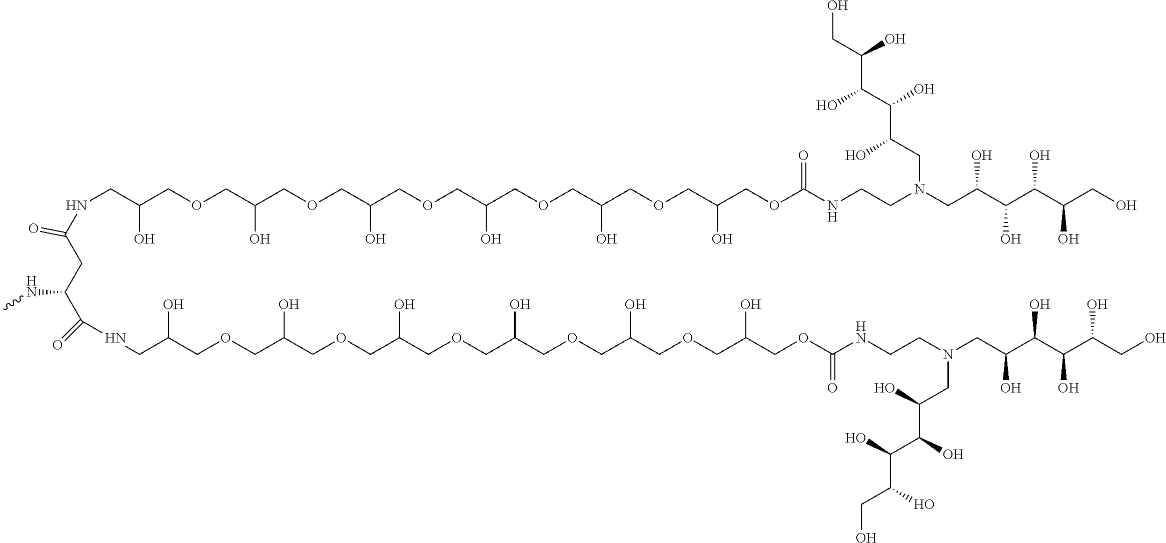 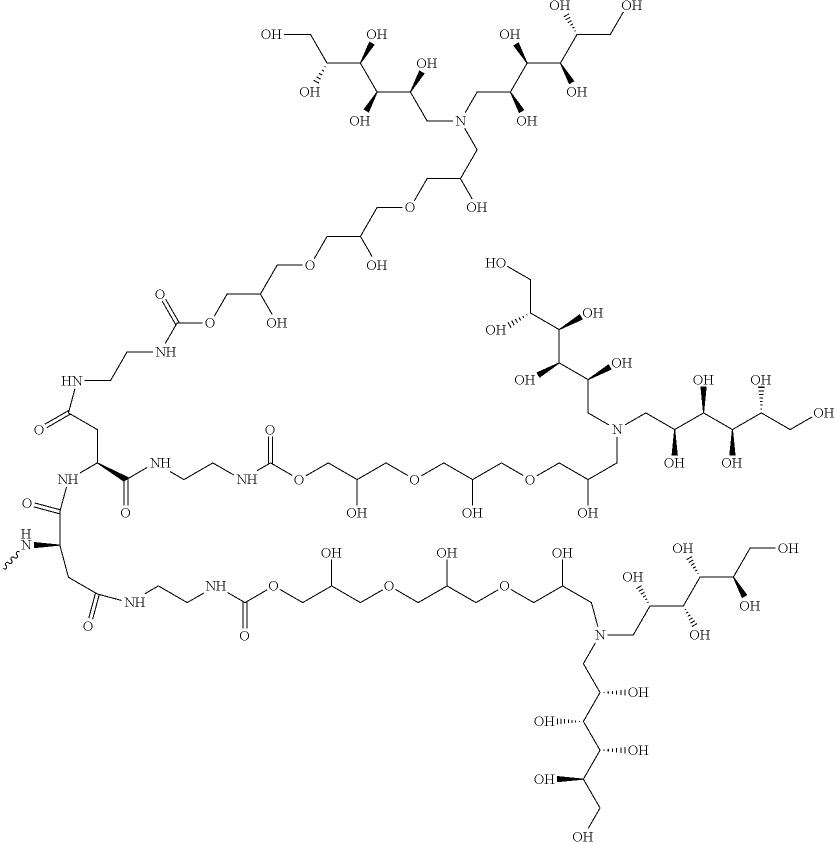 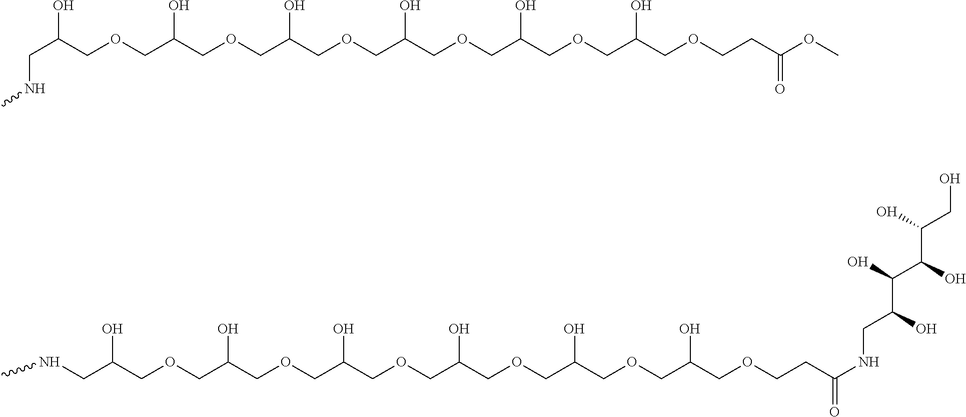 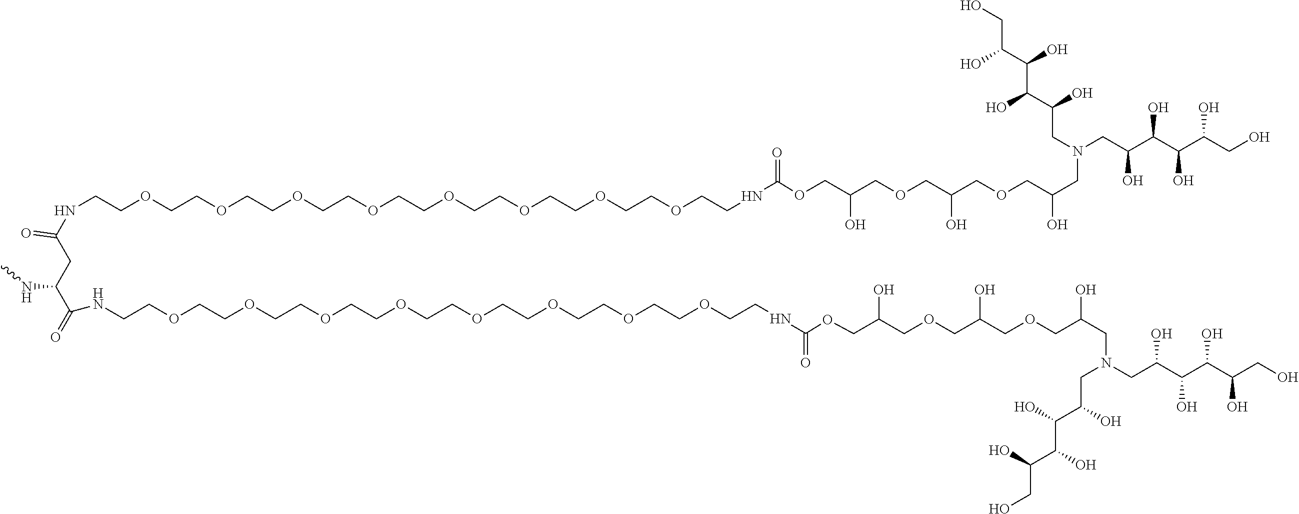 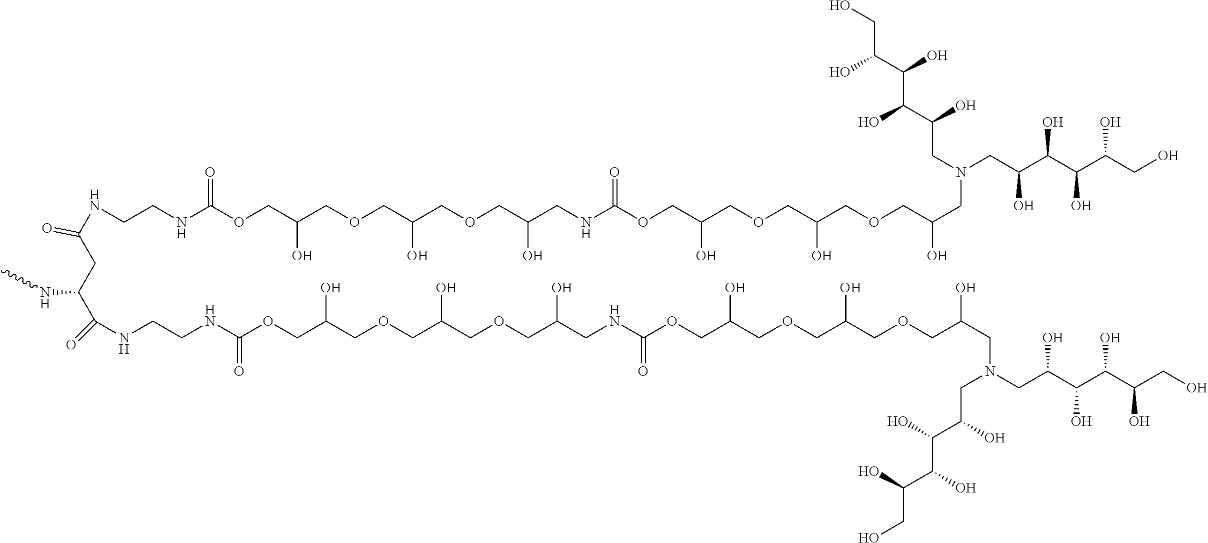 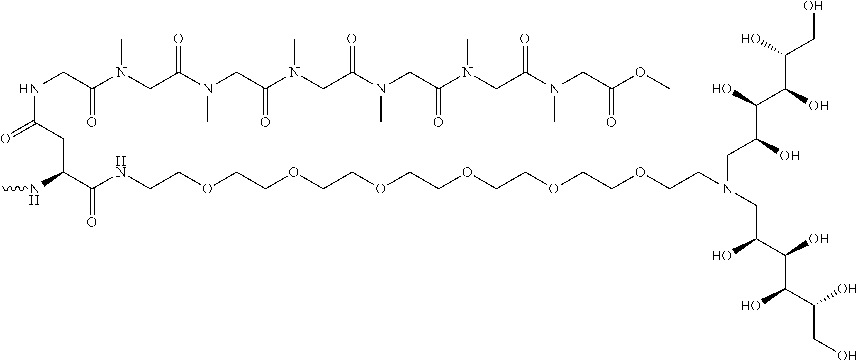 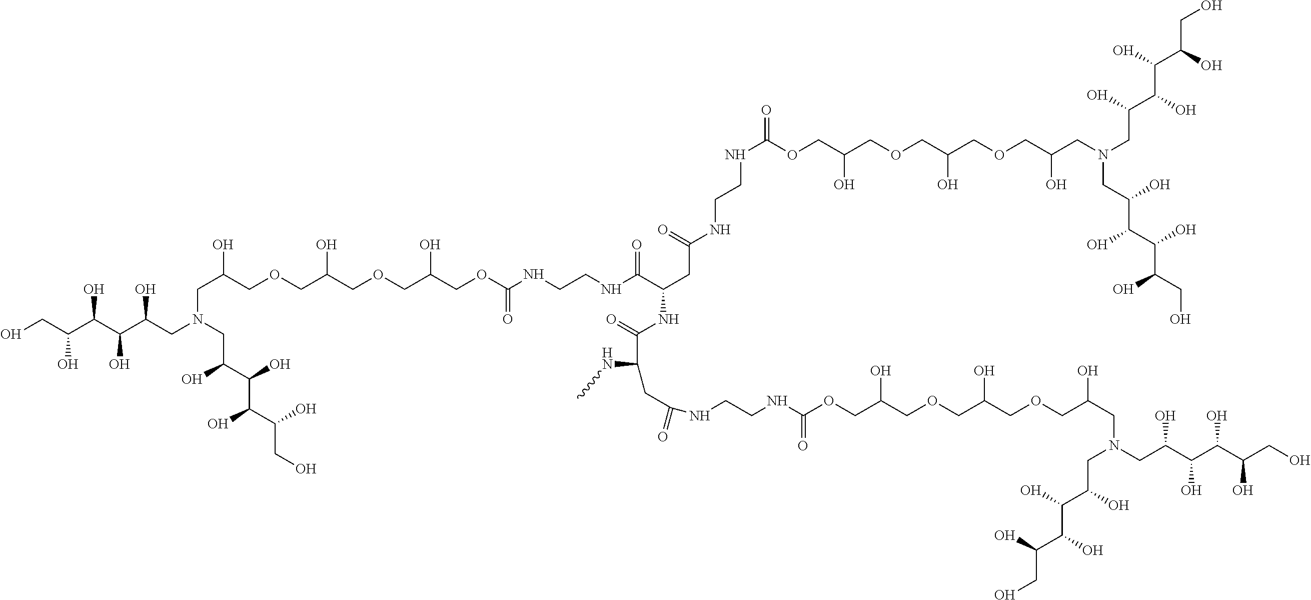 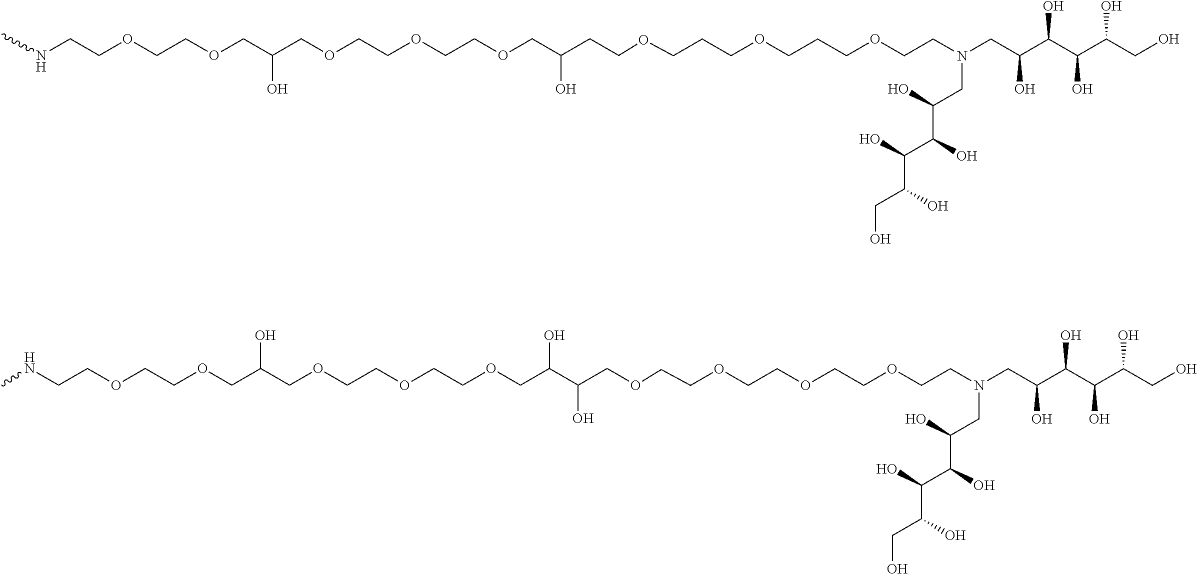 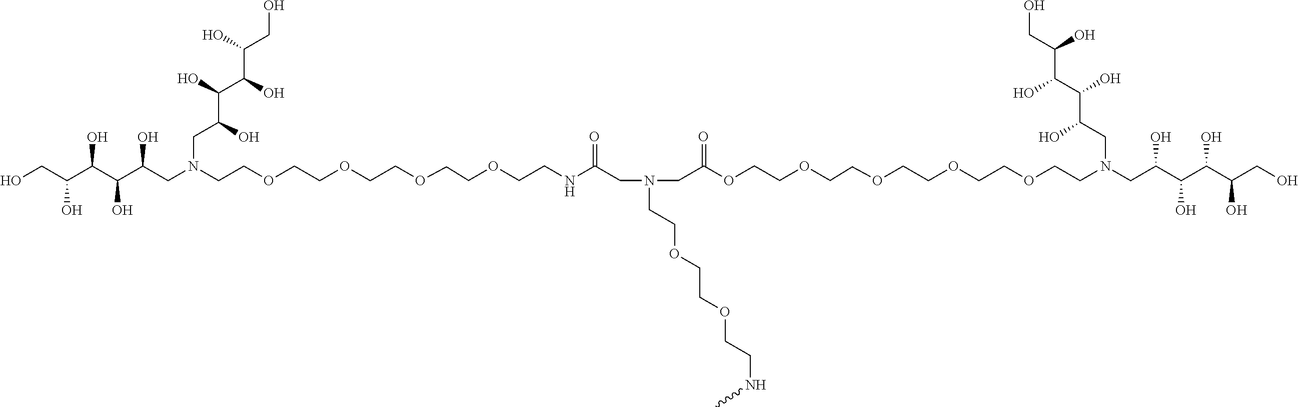 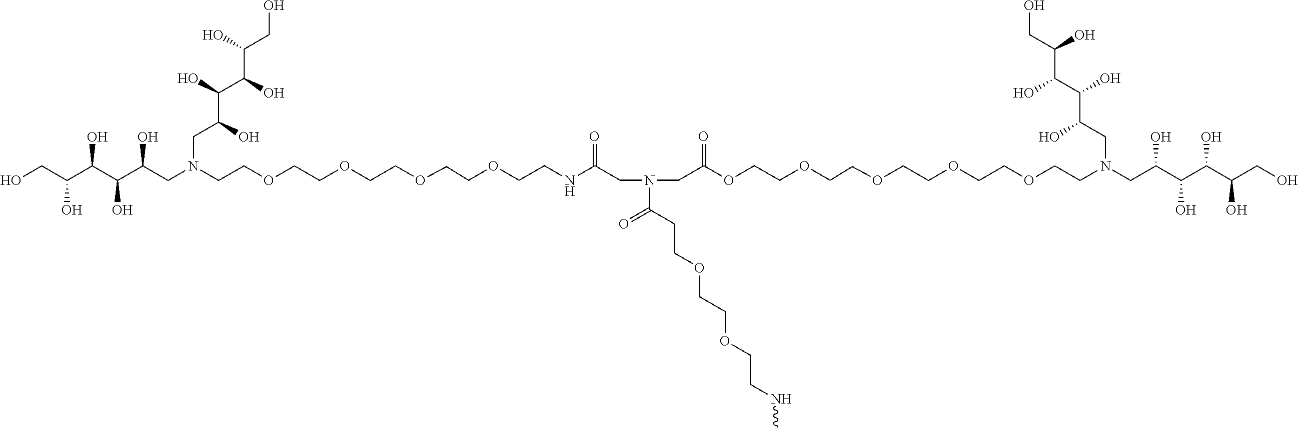 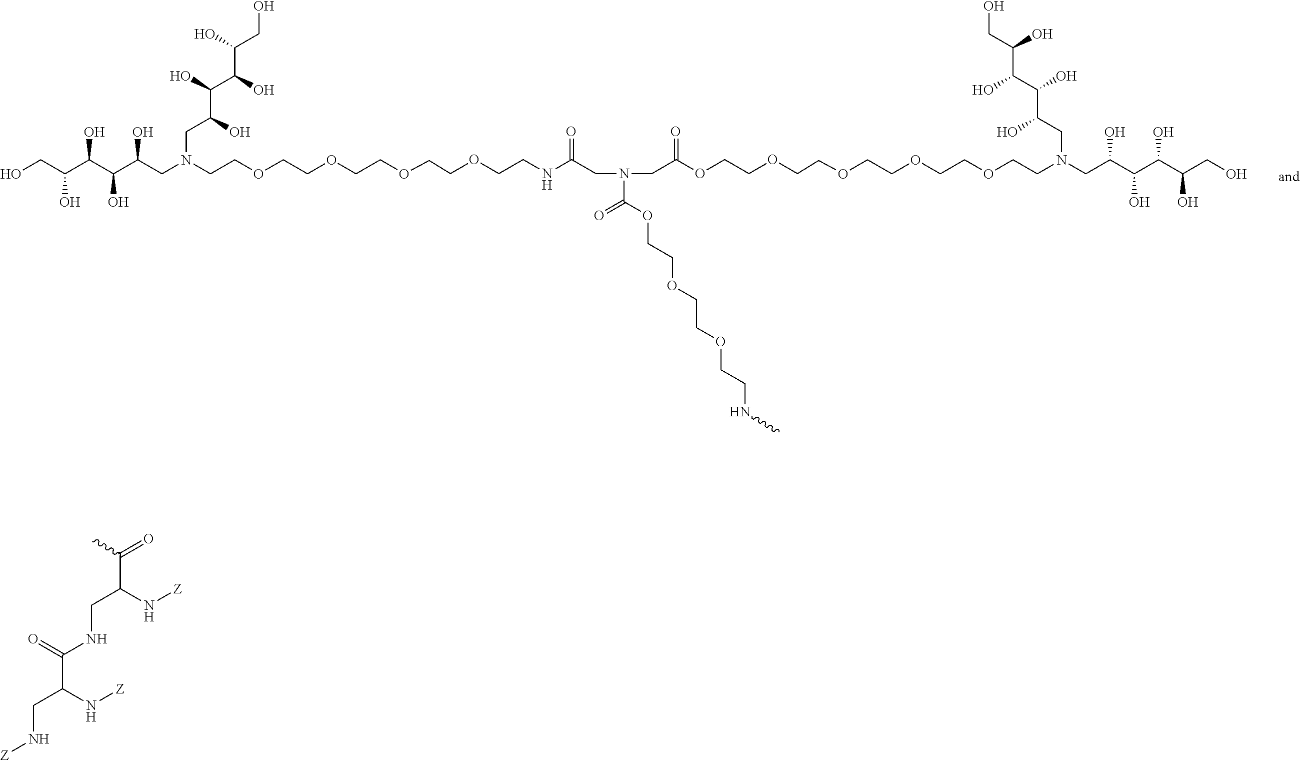 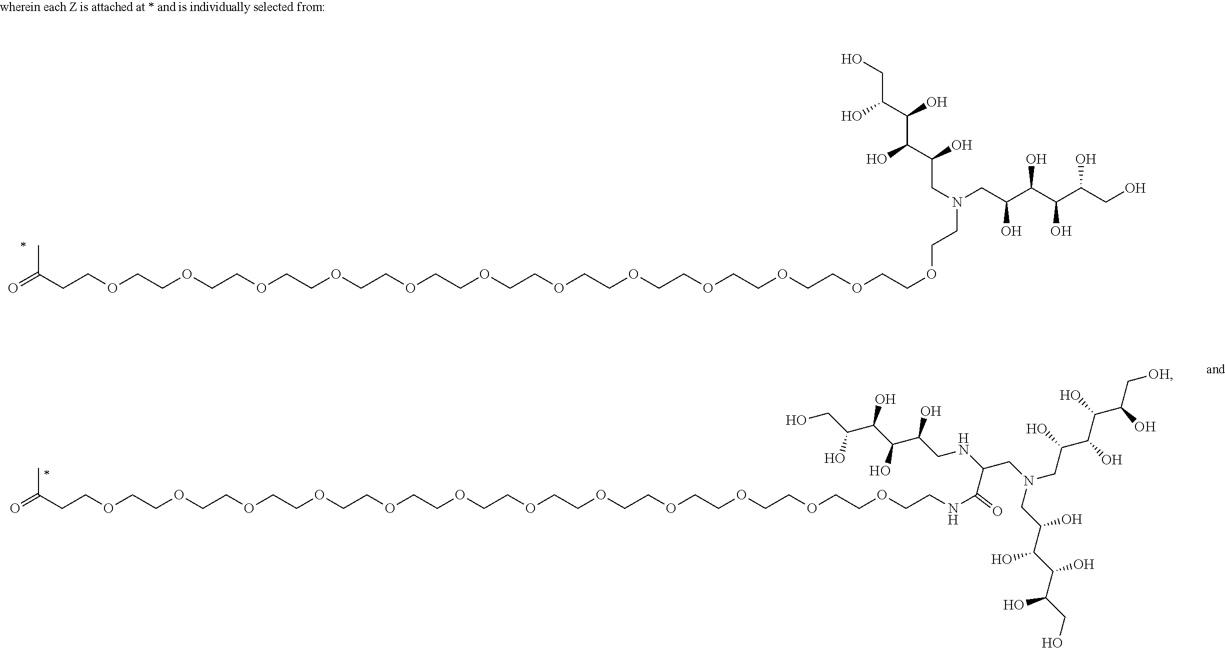 [SMILES: CC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO ; CC(=O)CCOCCOCCOCCOCCOCCOCCOCCOCCOCCOCCOCCOCCNC(=O)C(CN(C[C@H](O)[C@@H](O)[C@H](O)[C@H](O)CO)C[C@H](O)[C@@H](O)[C@H](O)[C@H](O)CO)NC[C@H](O)[C@@H](O)[C@H](O)[C@H](O)CO] 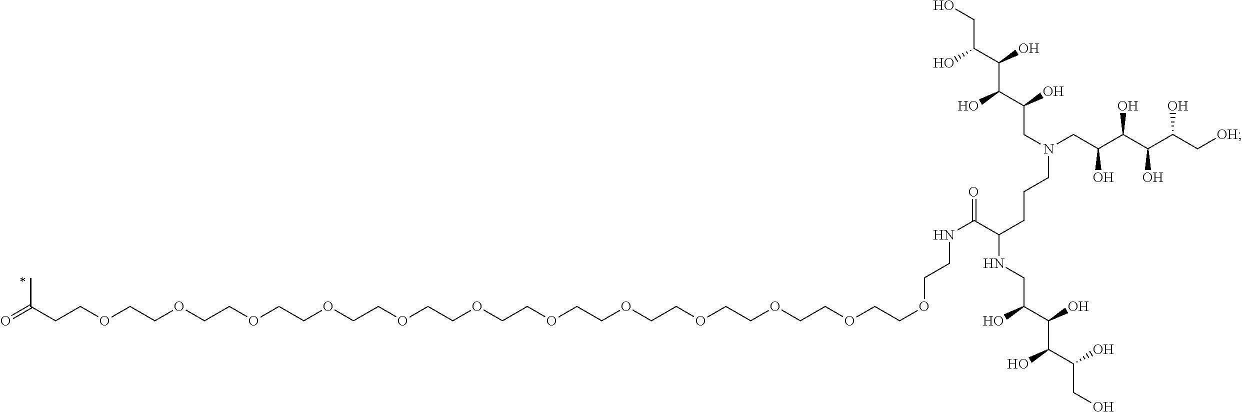 [SMILES: CC(=O)CCOCCOCCOCCOCCOCCOCCOCCOCCOCCOCCOCCOCCNC(=O)C(CCCN(C[C@H](O)[C@@H](O)[C@H](O)[C@H](O)CO)C[C@H](O)[C@@H](O)[C@H](O)[C@H](O)CO)NC[C@H](O)[C@@H](O)[C@H](O)[C@H](O)CO]
or a stereoisomer or salt thereof, wherein each [symbol] is an attachment to the Amino Acid unit.

69. The Linker compound of any one of claims 1-67, wherein the Polar group is selected from the following: 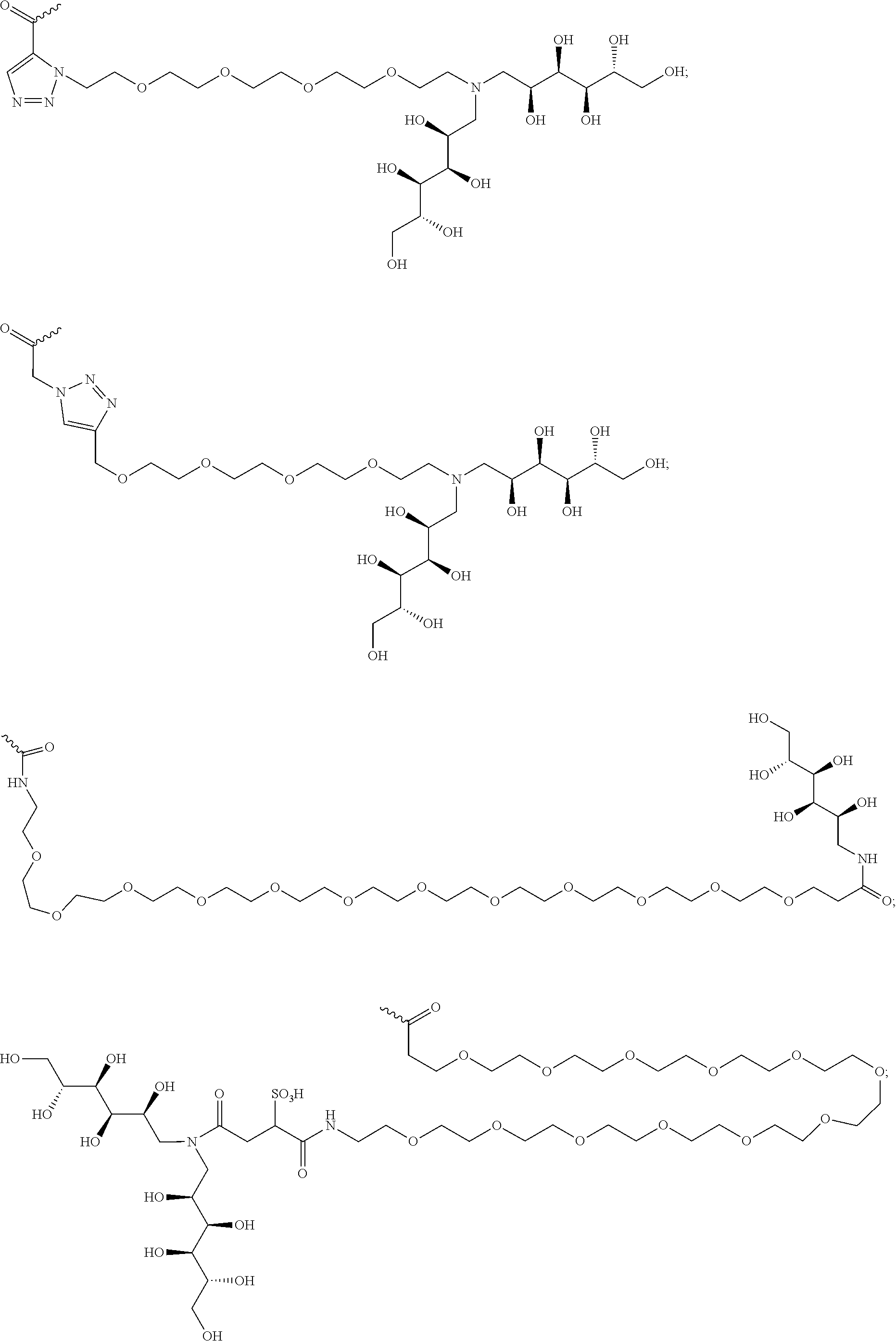 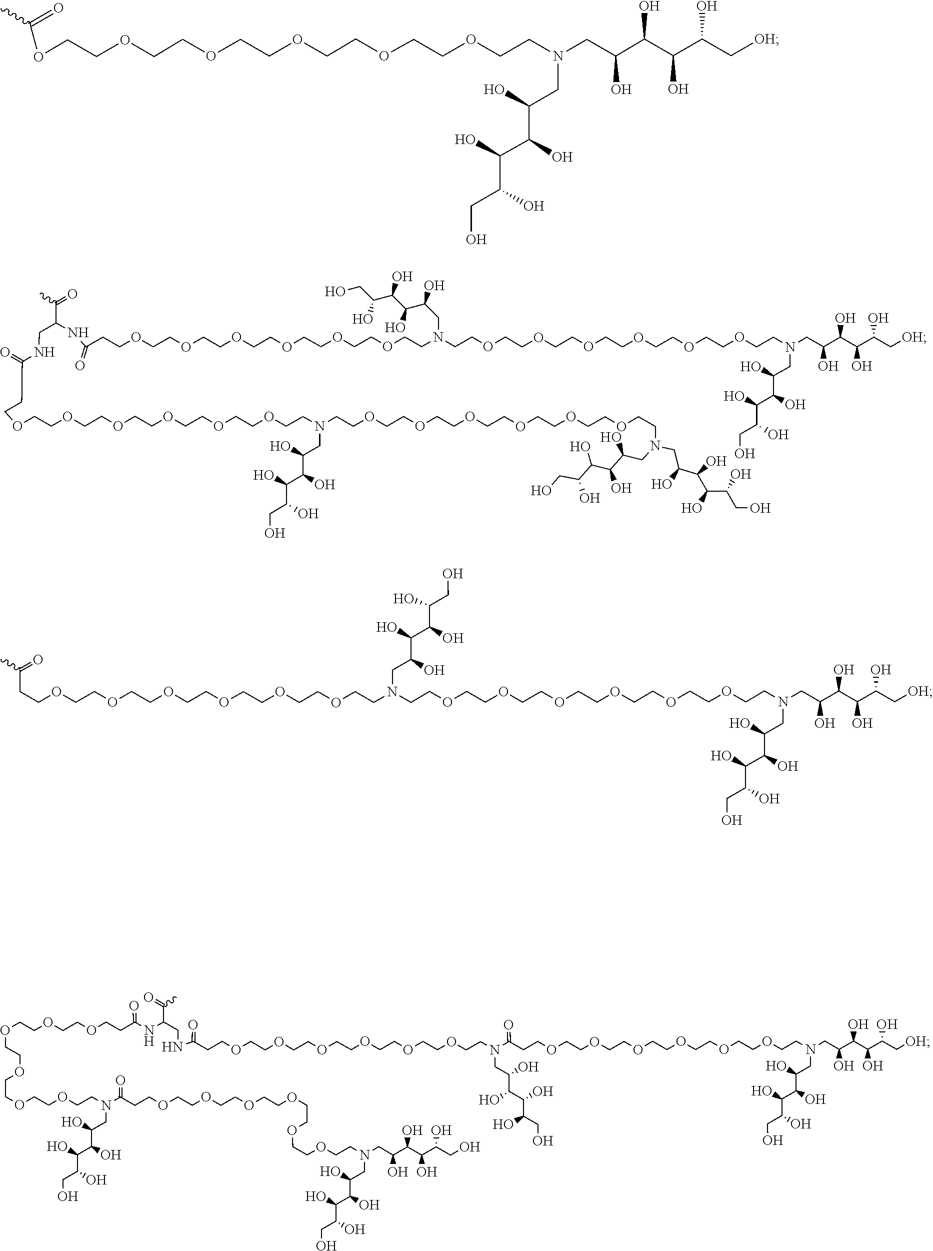 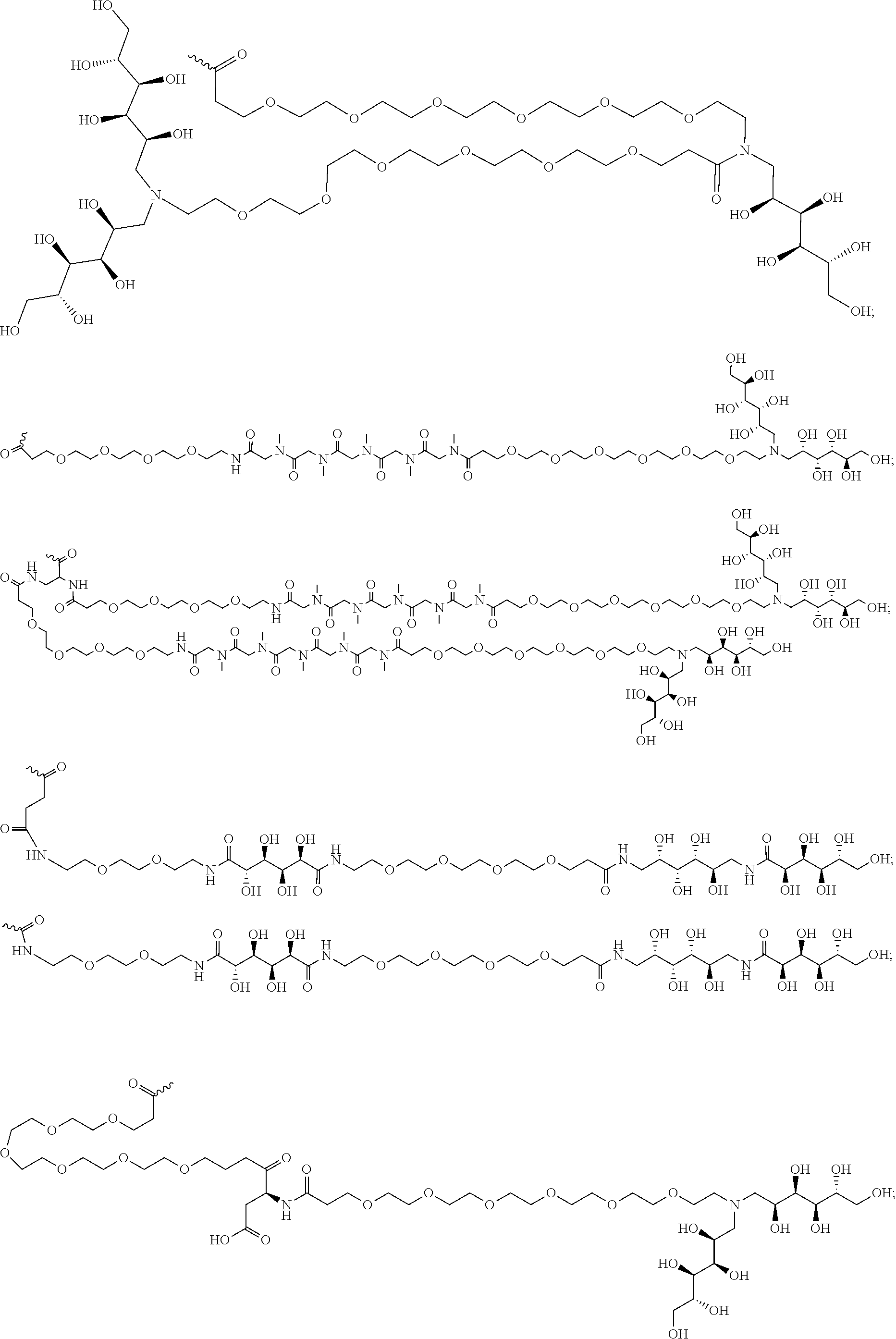 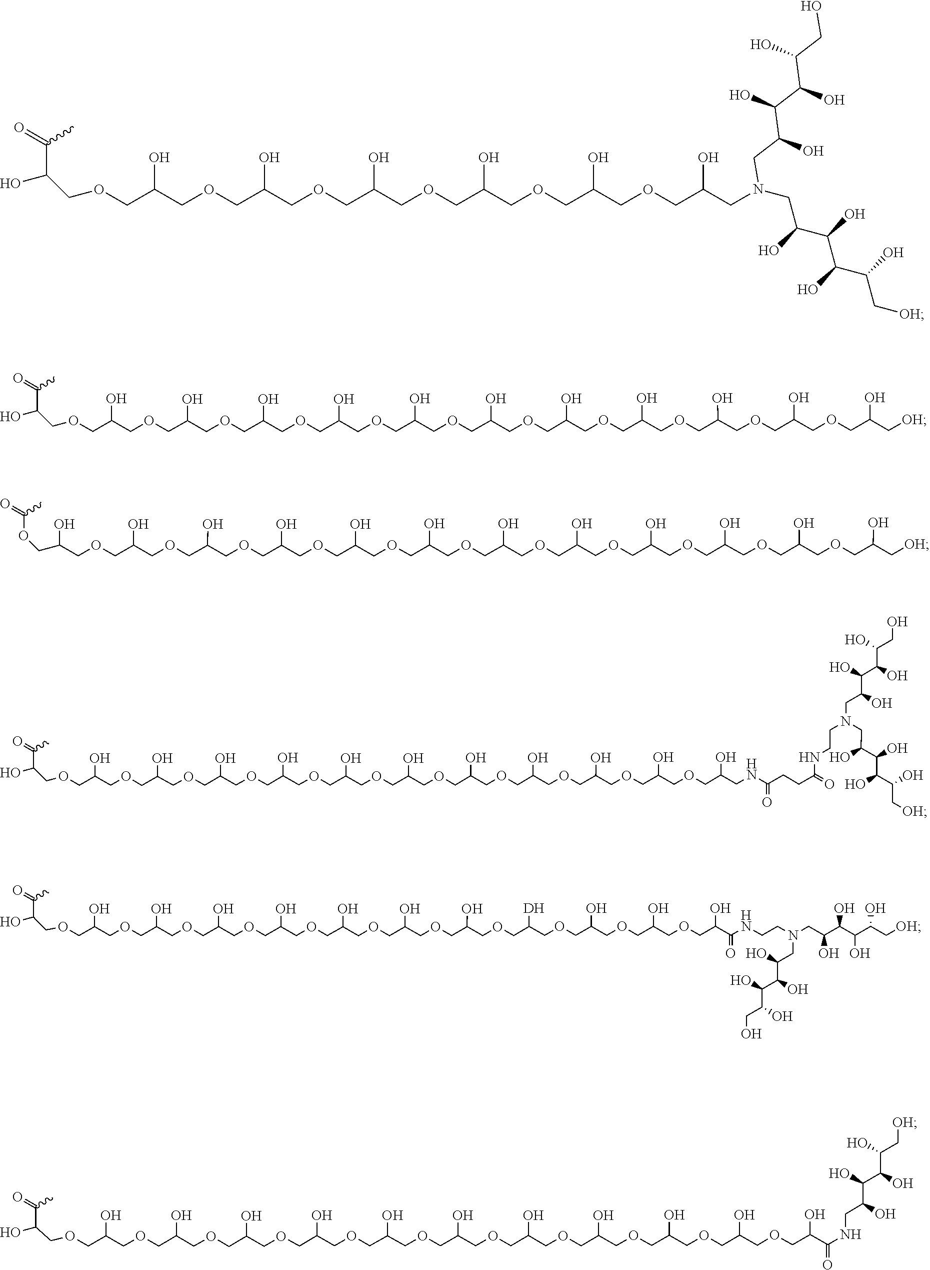 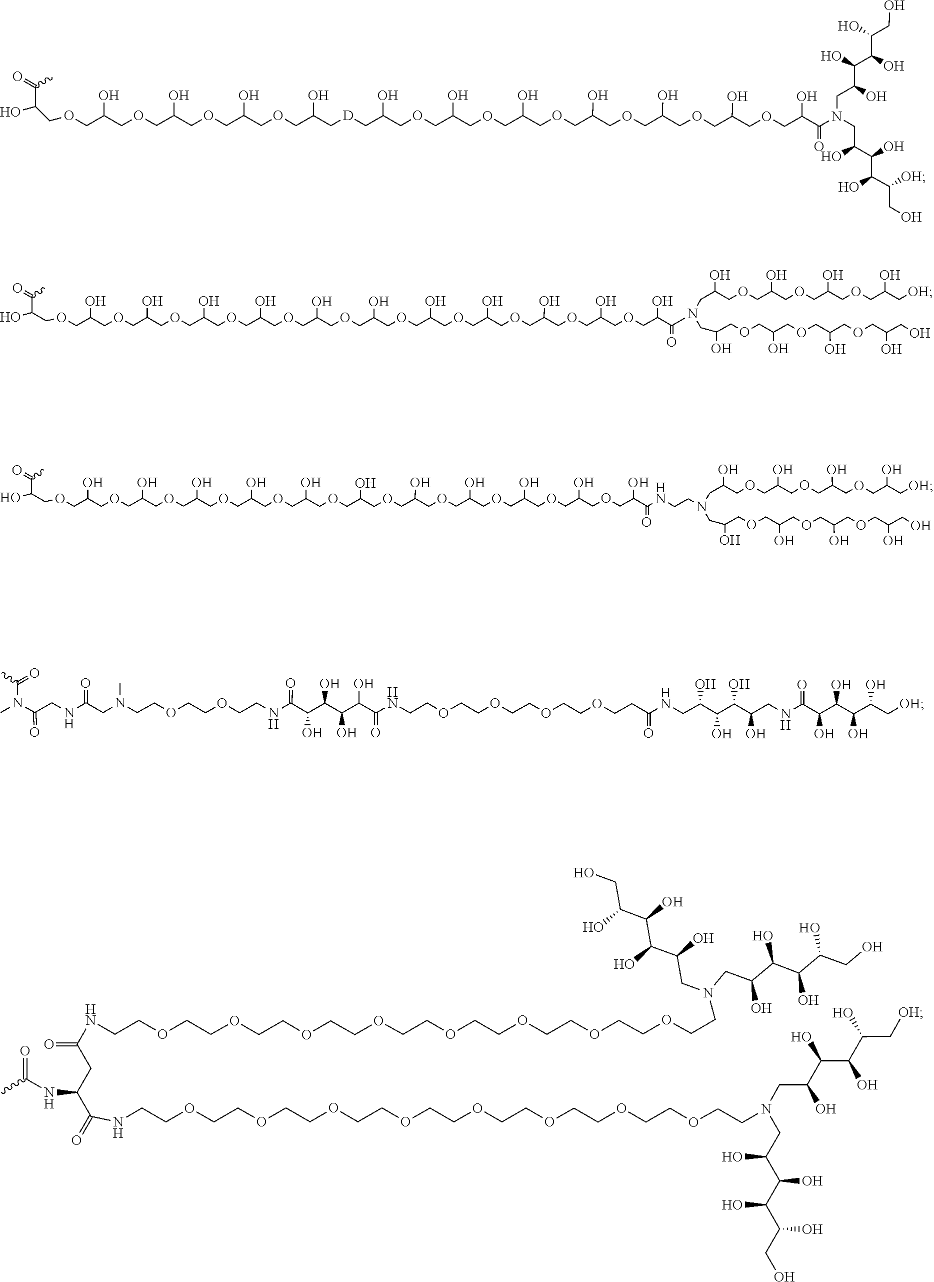 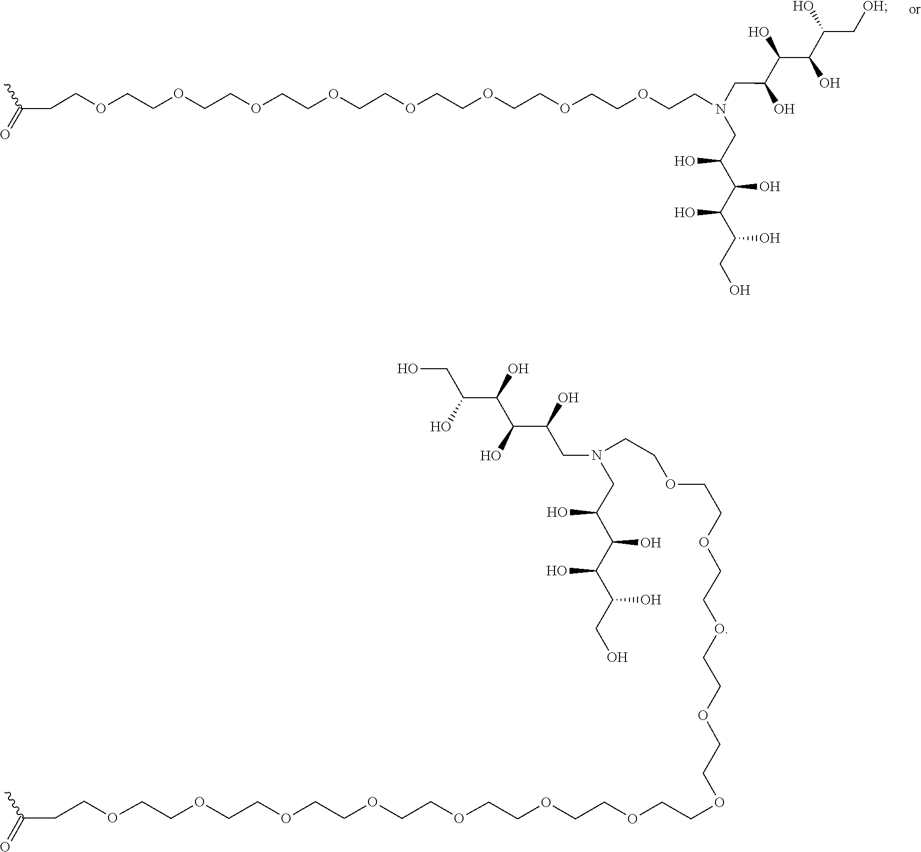
or a stereoisomer thereof, wherein each [symbol] indicates an attachment site of the Amino Acid unit.

70. The Linker compound of any one of claims 1-69, wherein the Polar group comprises at least one Carboxyl unit having the following formula: 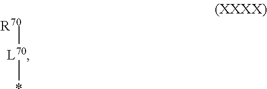
or a stereoisomer or salt thereof, wherein:
(a)
L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment to the Amino Acid unit, or to a remainder of the Polar group;
R⁷⁰ is ˜NR⁷¹(R⁷²—R⁷³), wherein R⁷¹ is selected from H, C₁-C₁₂ alkyl, substituted C₁-C₁₂ alkyl, or polyethylene glycol (optionally having 1 to 12 ethylene glycol subunits), R⁷² is a bond or is selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and R⁷³ is a carboxyl or polycarboxyl, wherein the polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide;
(b)
L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment to the Amino Acid unit, or to a remainder of the Polar group;
R⁷⁰ is ˜NR⁷¹(R⁷⁵—(R⁷³)₂), wherein R⁷¹ is selected from H, C₁-C₁₂ alkyl, substituted C₁-C₁₂ alkyl, or polyethylene glycol (optionally having 1 to 12 ethylene glycol subunits), R⁷⁵ is a branched optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl and each R⁷³ is independently carboxyl or polycarboxyl, wherein the polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide; or
(c)
L⁷¹ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment to the Amino Acid unit, or to a remainder of the Polar group;
R⁷⁰ is ˜N(R⁷⁴—R⁷³)(R²—R⁷³), wherein R⁷² and R⁷⁴ are each independently selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and each R⁷³ is independently carboxyl or polycarboxyl, wherein the polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide.

71. The Linker compound of any one of claims 1-70, comprising a Polar group including the Polymer unit and a Sugar unit.

72. The Linker compound of any one of claims 1-71, comprising a Polar group including at least two Polymer units.

73. The Linker compound of any one of claims 1-71, comprising a Polar group including the Polymer unit(s) and a Carboxyl unit.

74. The Linker compound of any one of claims 1-73, comprising at least two Polar groups.

75. The Linker compound of any one of claims 1-74, comprising a Polar group including the Polymer unit, the Sugar unit and the Carboxyl unit.

76. The Linker compound of any one of claims 1-75, comprising a Polar group including at least two Polymer units, at least one Sugar unit and at least one Carboxyl unit.

77. The Linker compound of any one of claims 1-76, wherein the Amino Acid unit comprises at least two amino acid subunits.

78. The Linker compound of any one of claims 1-77, comprising two of the Polar groups, when present, both attached to the Amino Acid unit.

79. The Linker compound of any one of claims 1-78, wherein the Linker unit is attached to a side chain of a subunit of the Amino Acid unit.

80. The Linker compound of any one of claims 1-79, wherein the Amino Acid unit is joined to the Linker Unit by a non-peptidic linking group.

81. The Linker compound of claim 80, wherein the non-peptidic linking group is selected from optionally-substituted C₁-C₁₀ alkylene, optionally-substituted C₂-C₁₀ alkenylene, optionally-substituted C₂-C₁₀ alkynylene, or optionally-substituted polyethylene glycol.

82. The Linker compound of any one of claims 1-81, comprising one of the following structures: 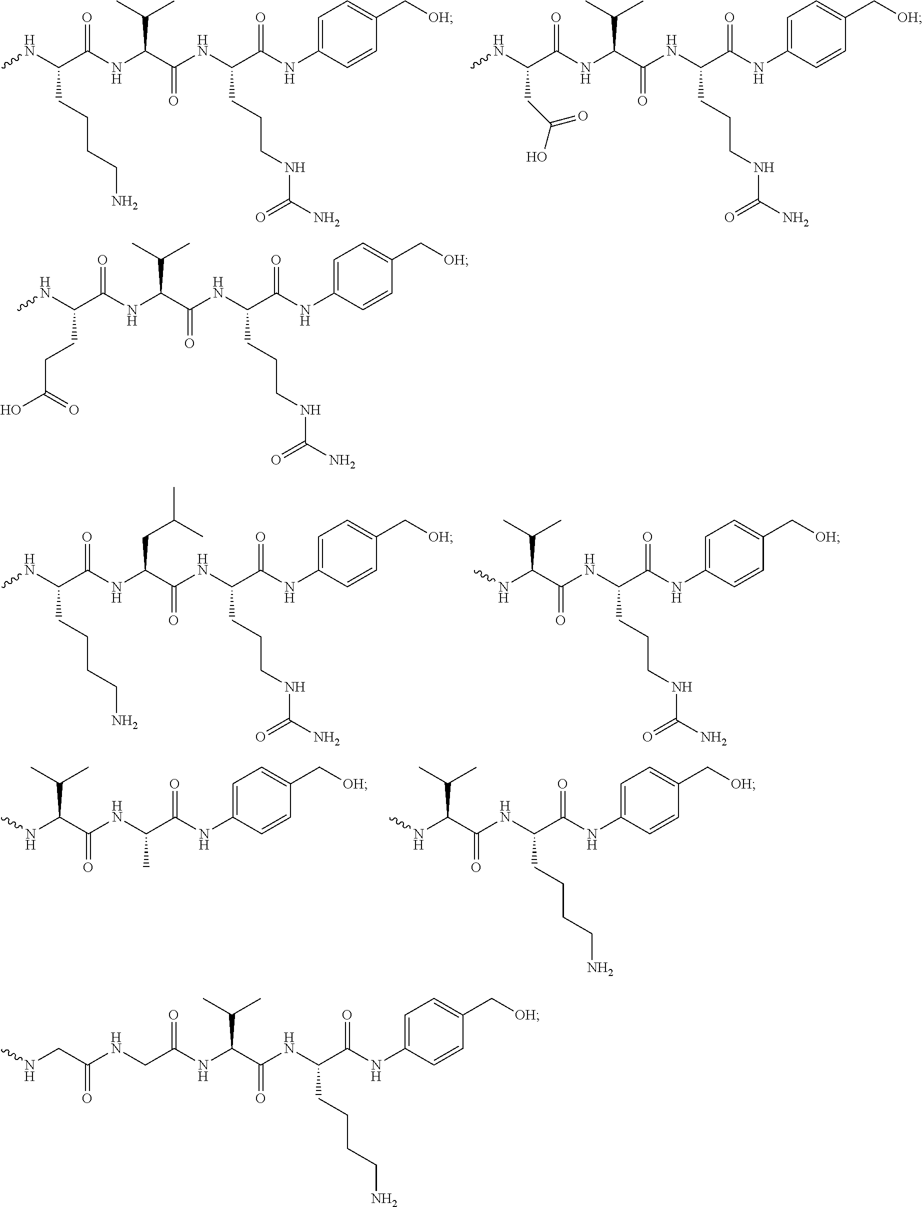 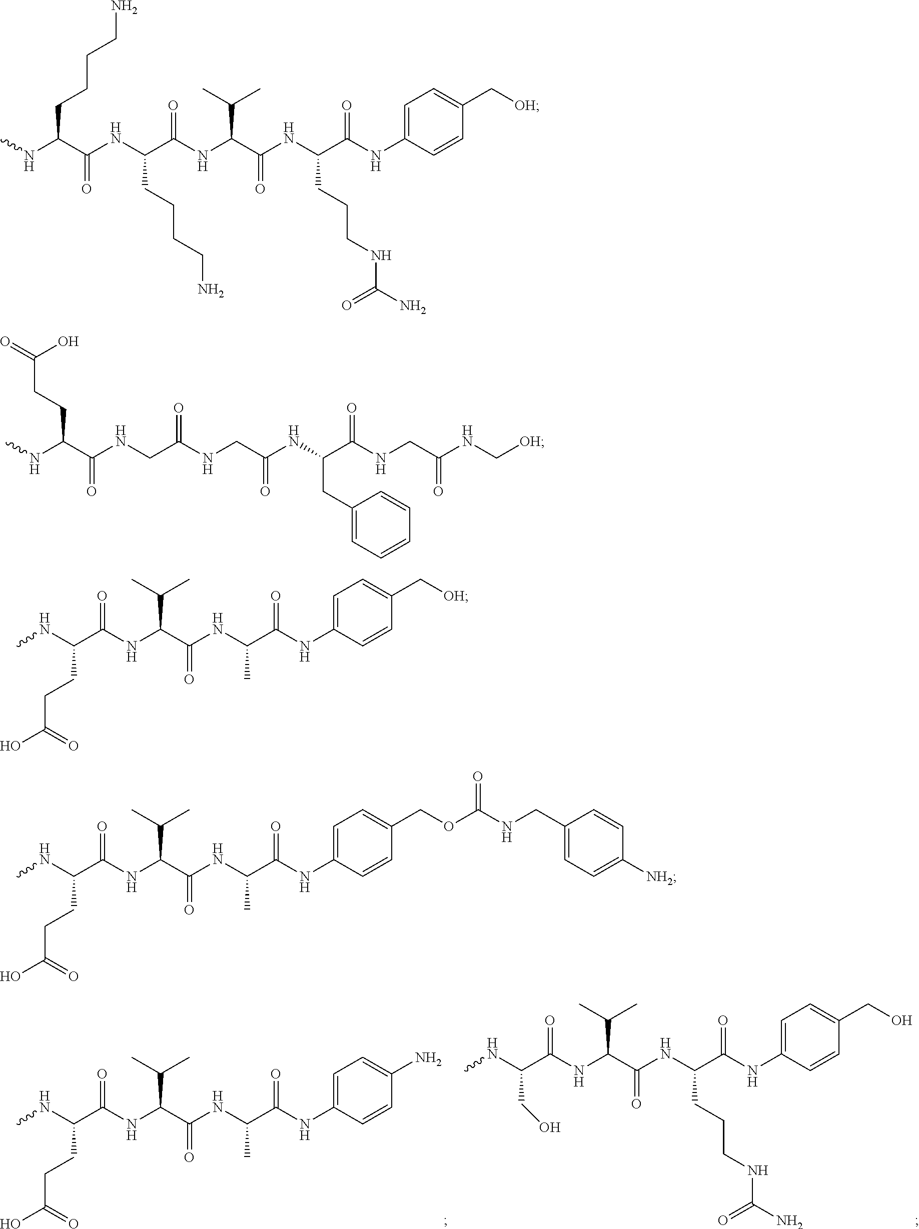 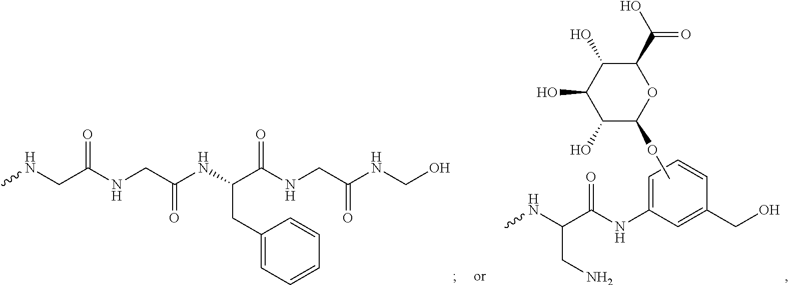
or a stereoisomer thereof, wherein the Polar group is attached to an amino acid subunit of the Amino Acid unit, the H of a hydroxyl or amino group of the para-aminobenzyl group or the H of a hydroxyl of the glycine residue of a GGFG peptide is optionally replaced with a bond to at least one of the Drug units, or to a linking group attached to the at least one of the Drug units, the wavy line on the amino group indicates an attachment site for a Stretcher unit or an Amino Acid unit or, prior to attachment, indicates H.

83. The Linker compound of any one of claims 1-82, comprising a formula selected from the following: 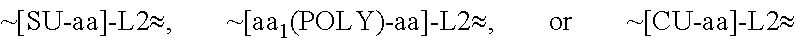
wherein the square brackets indicate the Amino Acid unit, each aa is an optional subunit of the Amino Acid unit, L2 is the Linker unit, each wavy line (˜) indicates an attachment site for a Stretcher unit; aa₁(POLY) is a Polymer unit attached to an amino acid subunit of the Amino Acid unit, SU is a Sugar unit attached to a subunit of the Amino Acid unit or to the Linker unit, and CU is a Carboxyl unit attached to a subunit of the Amino Acid unit or to the Linker unit; and the double wavy (≈) line indicates an attachment site for at least one of the Drug units, wherein aa and aa₁ are independently selected from alpha, beta and gamma amino acids and derivatives thereof.

84. The Linker compound of any one of claims 1-82, comprising a formula selected from the following: 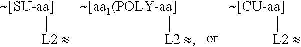
wherein the square brackets indicate the Amino Acid unit, each aa is an amino acid subunit of the Amino Acid unit, L2 is the Linker Subunit attached to a side chain of aa, the wavy line (˜) indicates an attachment site for a Stretcher unit; aa₁(POLY) is a Polymer unit attached to aa, SU is a Sugar unit attached to aa, CU is a Carboxyl unit attached to aa, and the double wavy (≈) line indicates an attachment site for at least one of the Drug units; wherein aa and aa₁ are independently selected from alpha, beta and gamma amino acids and derivatives thereof.

85. The Linker compound of any one of claims 1-82, wherein at least two Polymer units are attached to the Amino Acid unit.

86. The Linker compound of any one of claims 1-82, comprising a formula selected from the following: 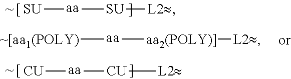
wherein the square brackets indicate the Amino Acid unit, an is an optional subunit of the Amino Acid unit, L2 is the Linker unit, the wavy line (˜) indicates an attachment site for a Stretcher unit; each of aa₁(POLY) and aa₂(POLY) is a Polymer unit attached to an or to the other Polymer unit; each SU is a Sugar unit attached to an or the other Sugar unit, each CU is a Carboxyl unit attached to an or to the other Carboxyl unit, and the double wavy (≈) line indicates an attachment site for at least one of the Drug units; wherein aa, aa₁ and aa₂ are independently selected from alpha, beta and gamma amino acids and derivatives thereof.

87. The Linker compound of any one of claims 1-82, comprising a formula selected from the following: 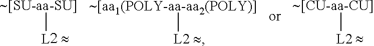
wherein the square brackets indicate the Amino Acid unit, an is an amino acid subunit of the Amino Acid unit, L2 is a Linker unit attached to a side chain of aa, each wavy line (˜) indicates an attachment site for a Stretcher unit; each of aa₁(POLY) and aa₂(POLY) is a Polymer unit attached to aa, each SU is a Sugar unit attached to aa; each CU is a Carboxyl unit attached to aa; and the double wavy (≈) line indicates an attachment site for at least one of the Drug units; wherein each of aa, aa₁ and aa₂ is independently selected from alpha, beta and gamma amino acids and derivatives thereof.

88. The Linker compound of any one of claims 1-87, wherein the Linker Unit is a cleavable linker unit.

89. The Linker compound of claim 88, wherein the Linker Unit comprises a peptide that is cleavable by an intracellular protease.

90. The Linker compound of claim 89, wherein the cleavable peptide comprises a valine-citrulline peptide, a valine-alanine peptide, a valine-lysine peptide, a phenylalanine-lysine peptide, or a glycine-glycine-phenylalanine-glycine peptide.

91. The Linker compound of any one of claims 1-87, wherein the Amino Acid unit comprises a peptide that is cleavable by an intracellular protease.

92. The Linker compound of claim 91, wherein the cleavable peptide comprises a valine-citrulline peptide, a valine-alanine peptide, a valine-lysine peptide, a phenylalanine-lysine peptide, or a glycine-glycine-phenylalanine-glycine peptide.

93. The Linker compound of any one of claims 88-92, wherein the cleavable peptide is attached to a para-aminobenzyl alcohol self immolative group (PABA).

94. The Linker compound of any one of claims 1-93, comprising one of the following 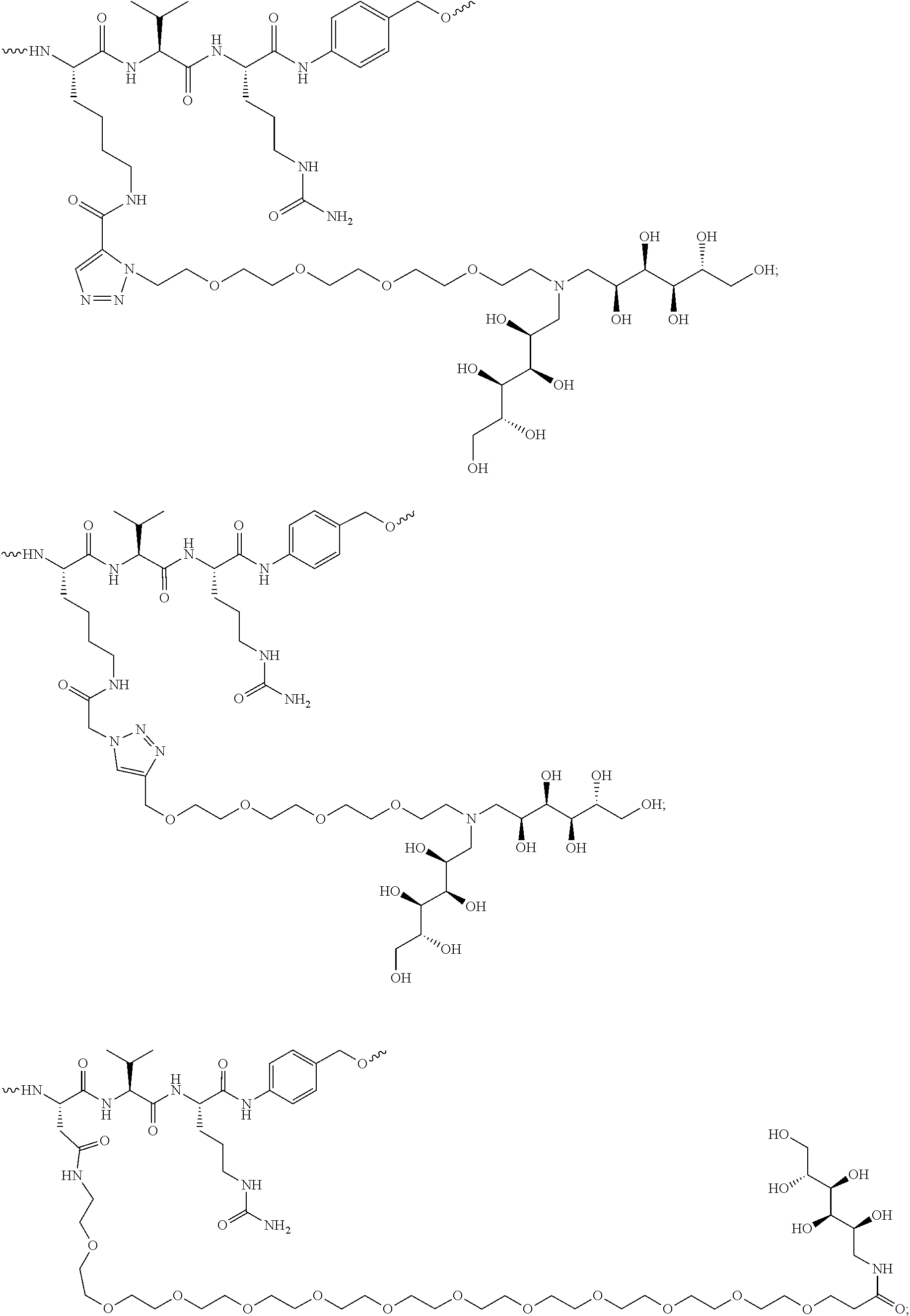 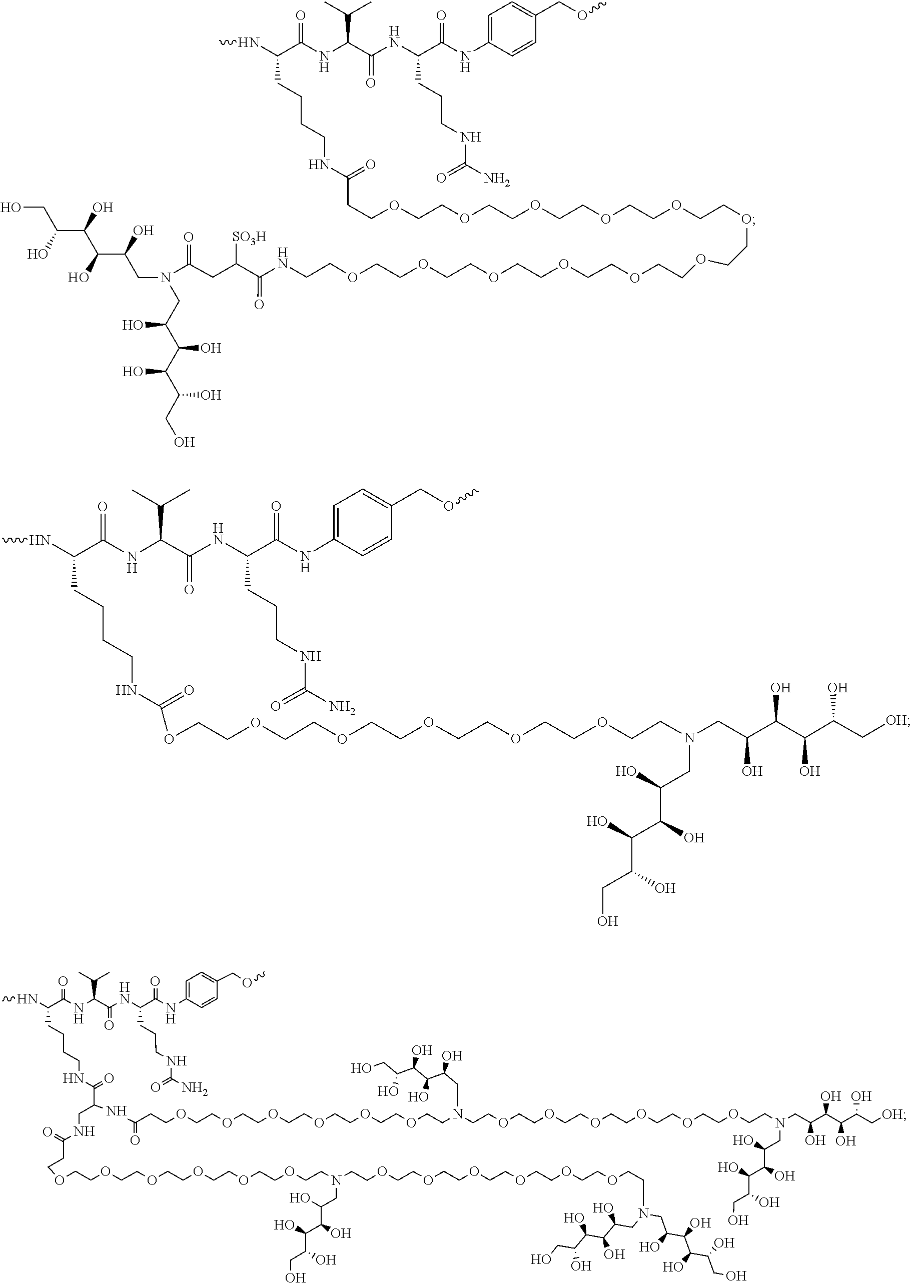 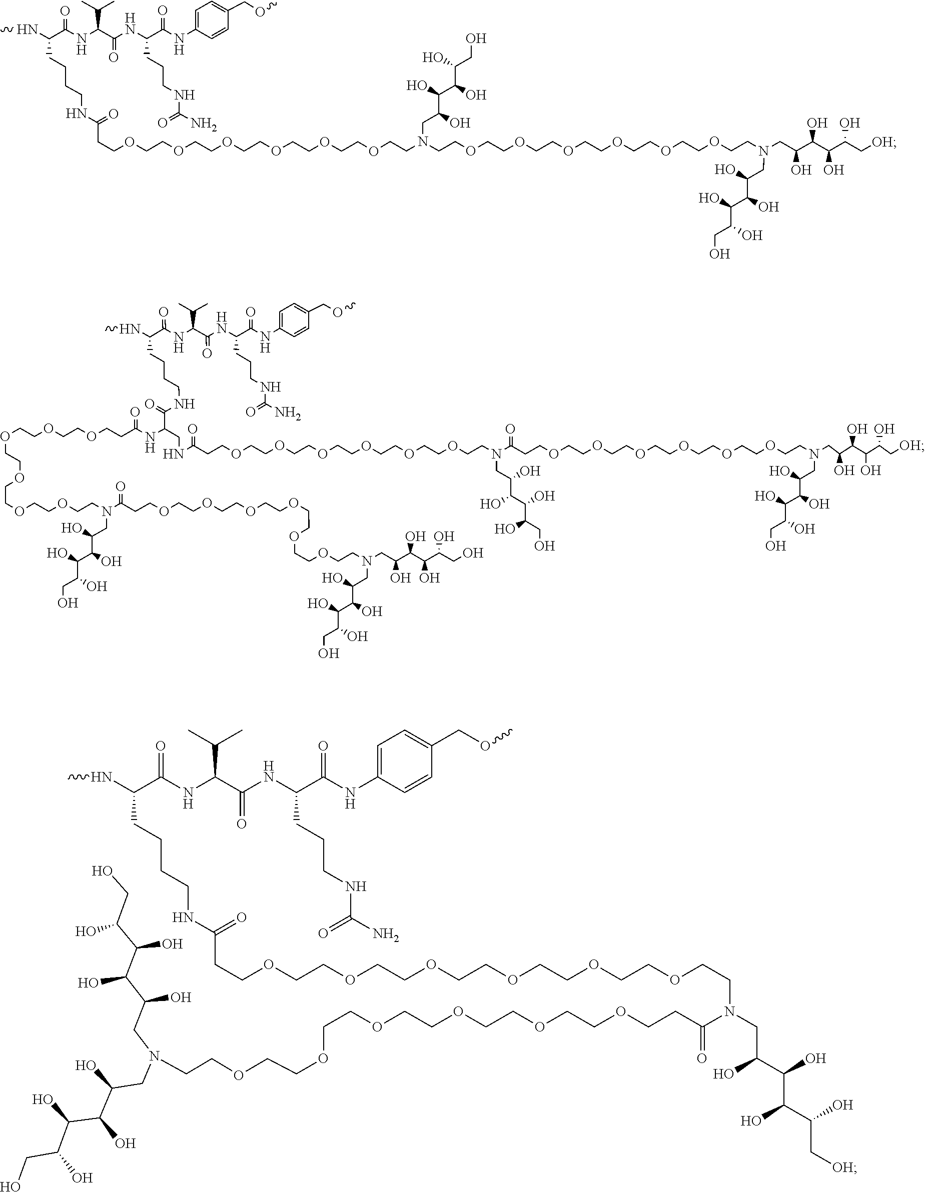 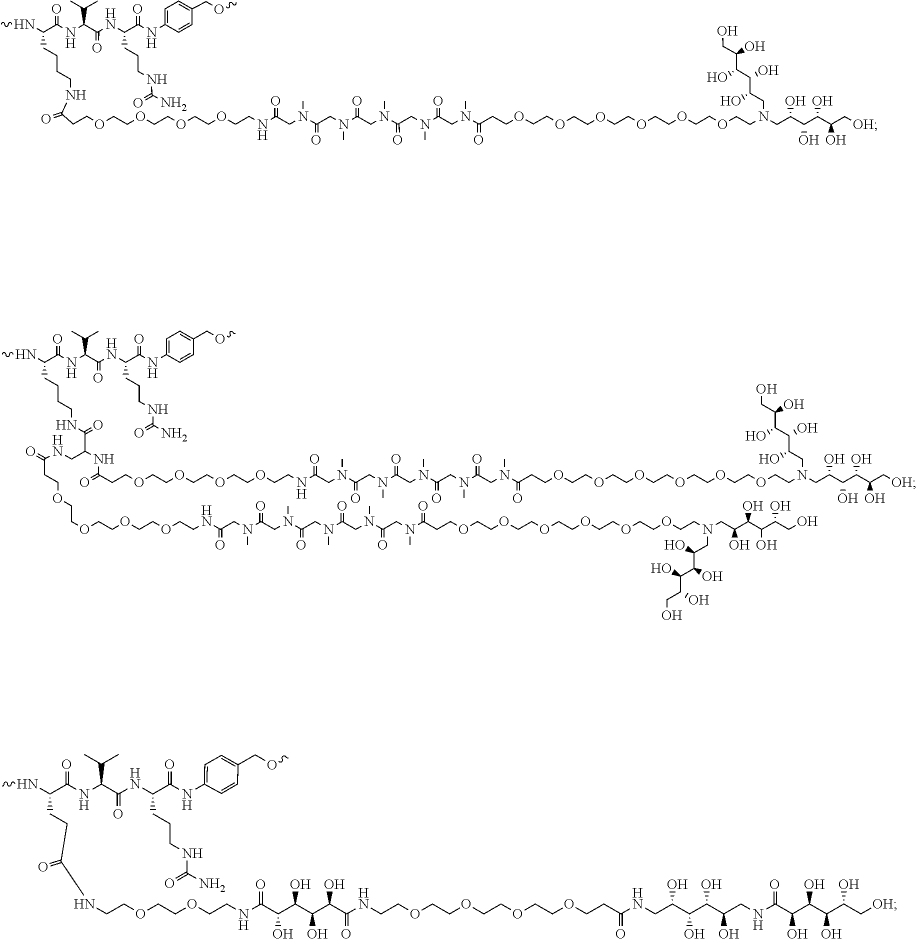 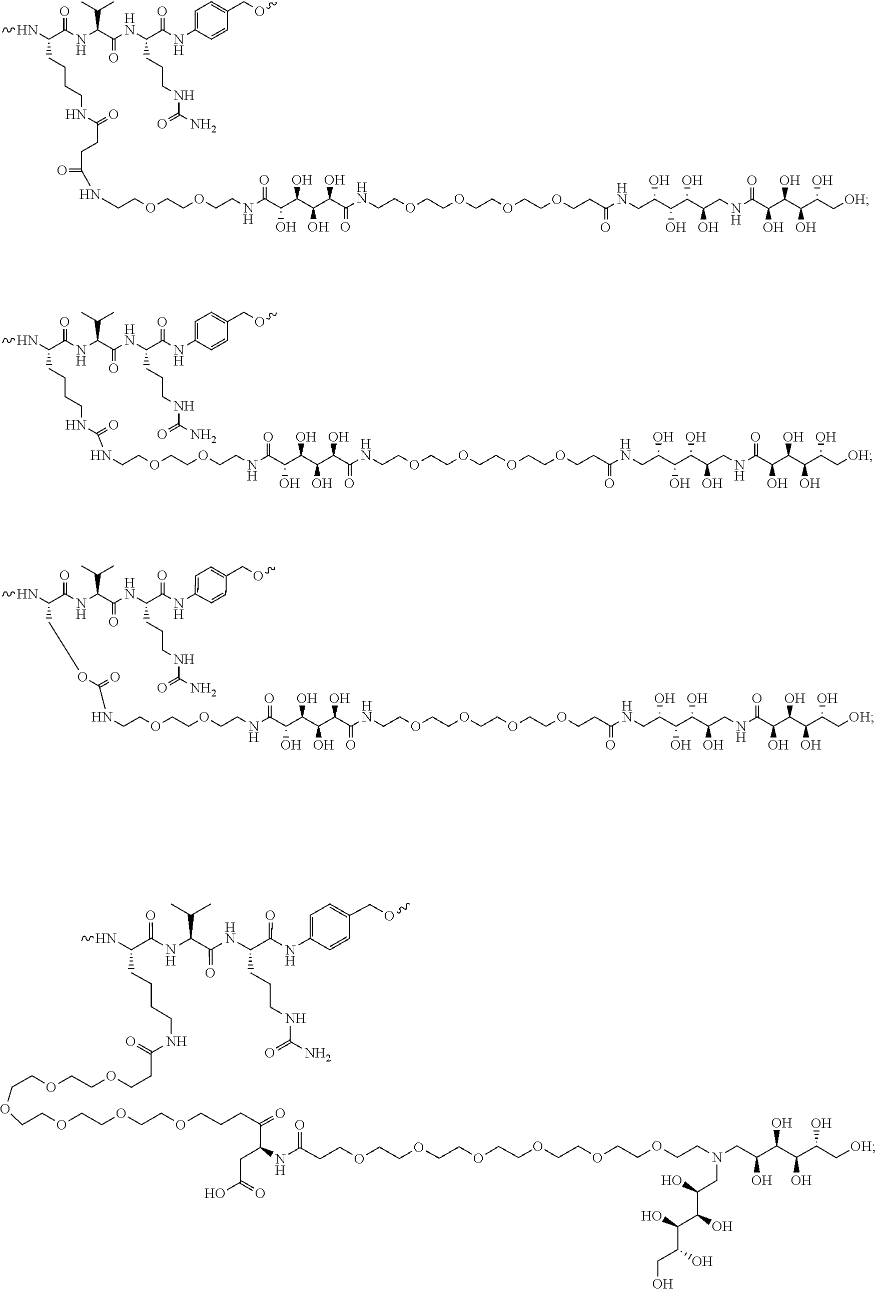 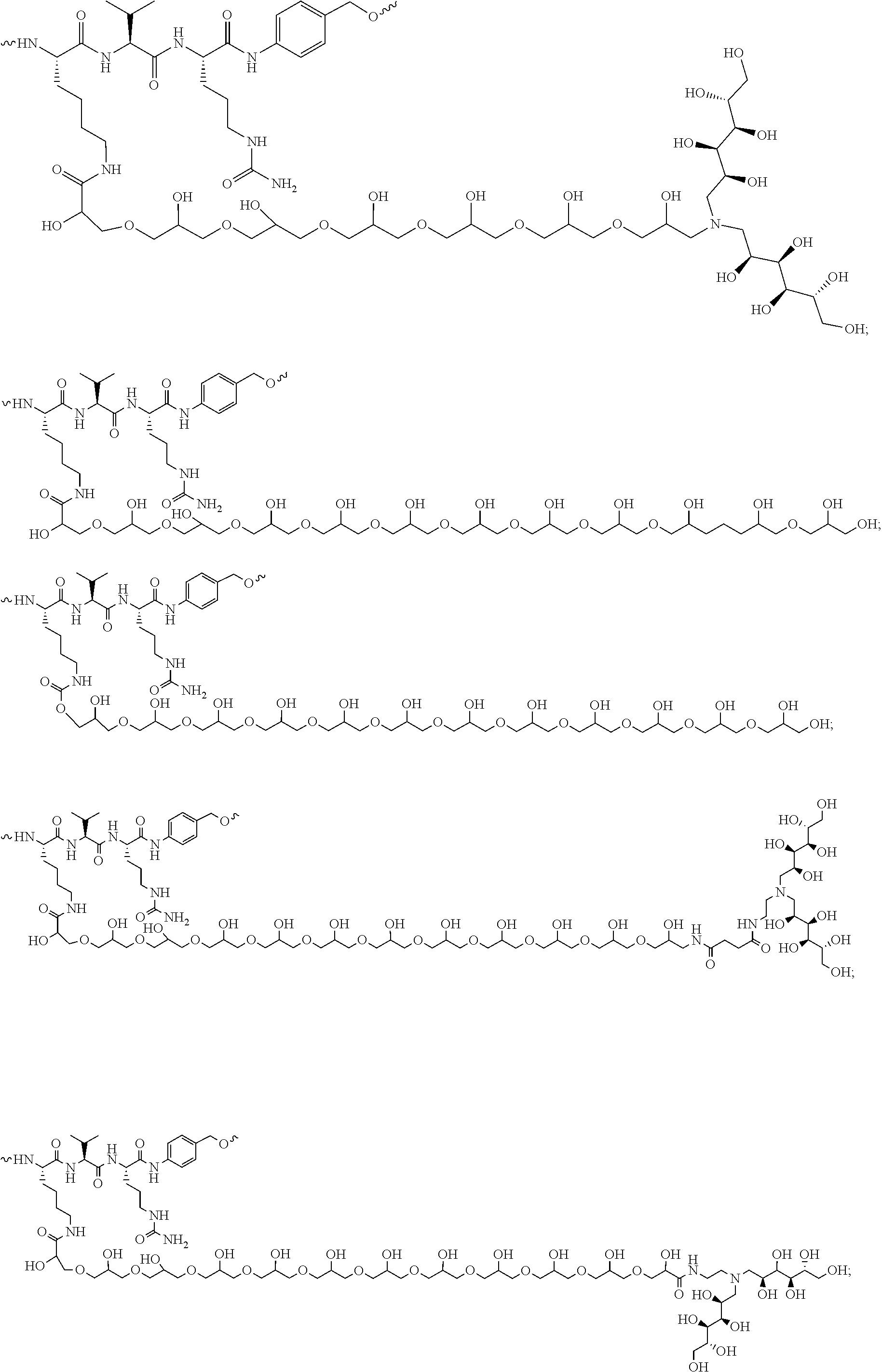 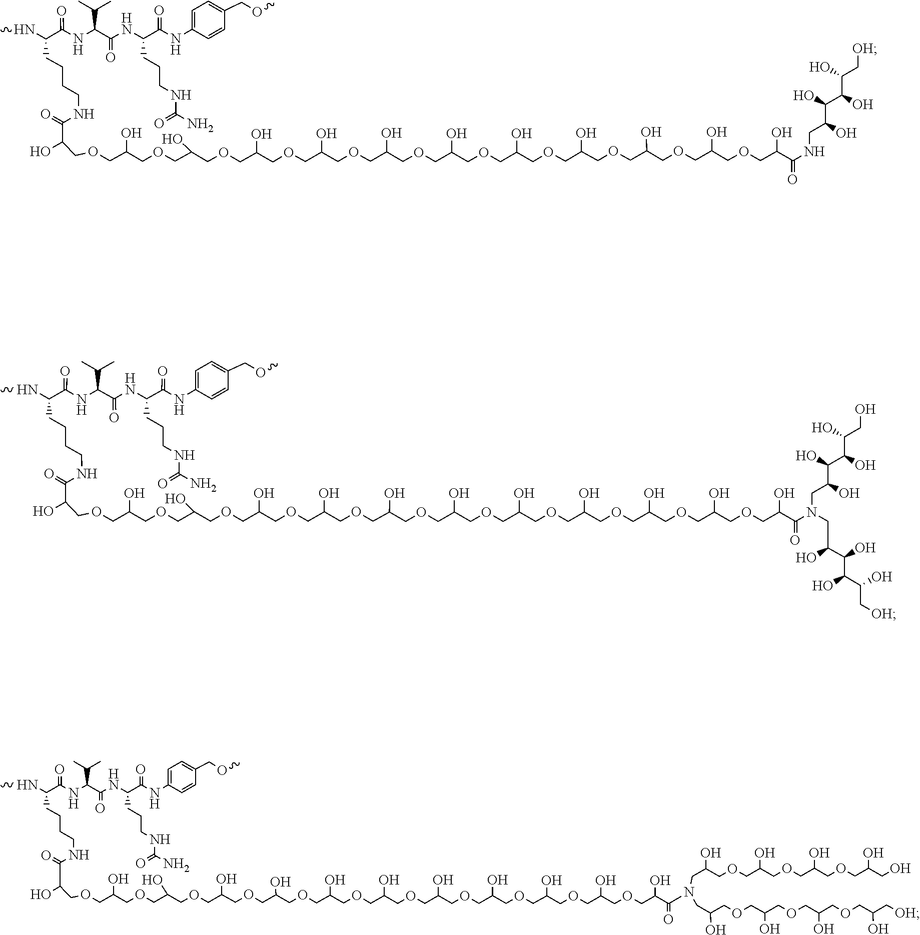 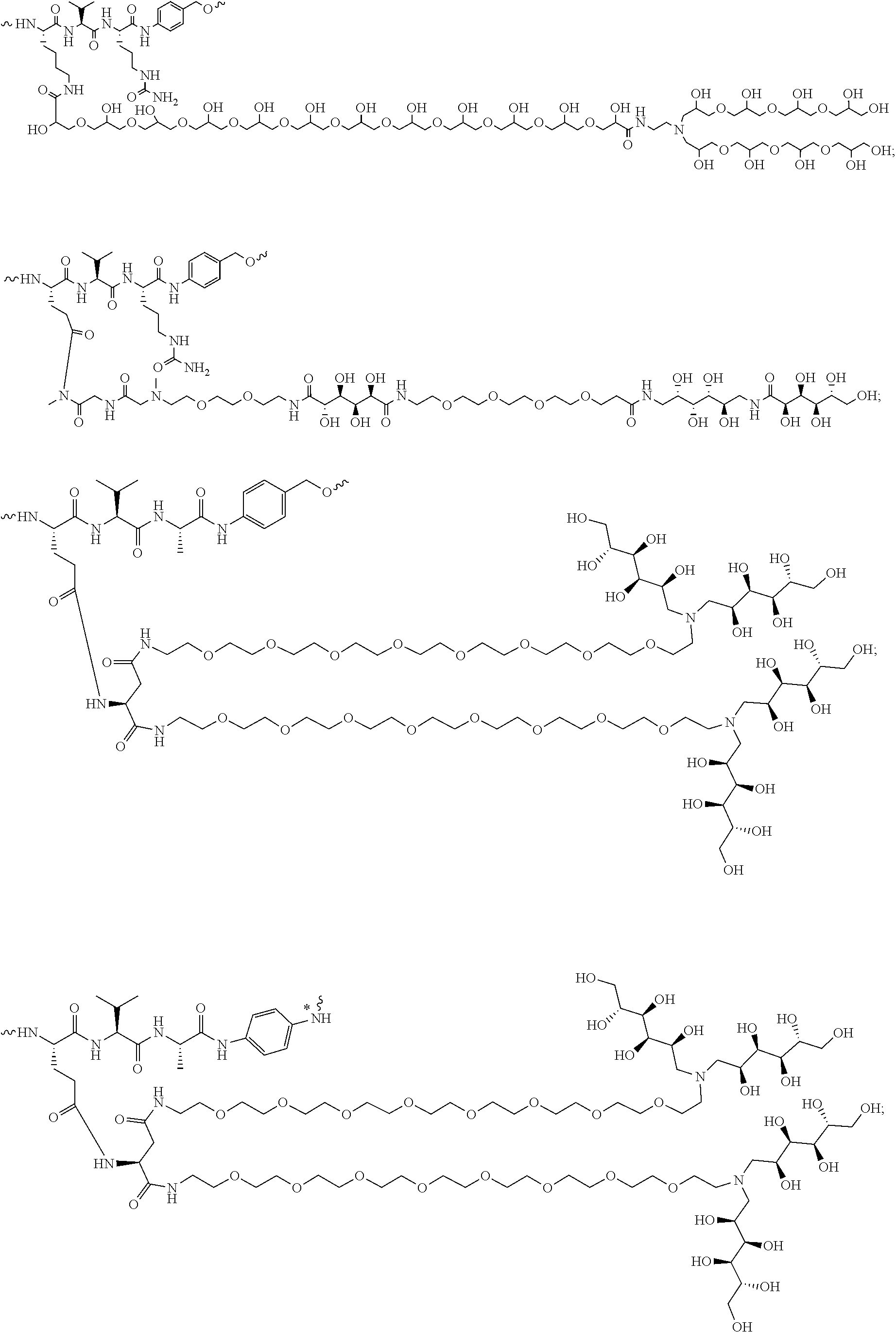 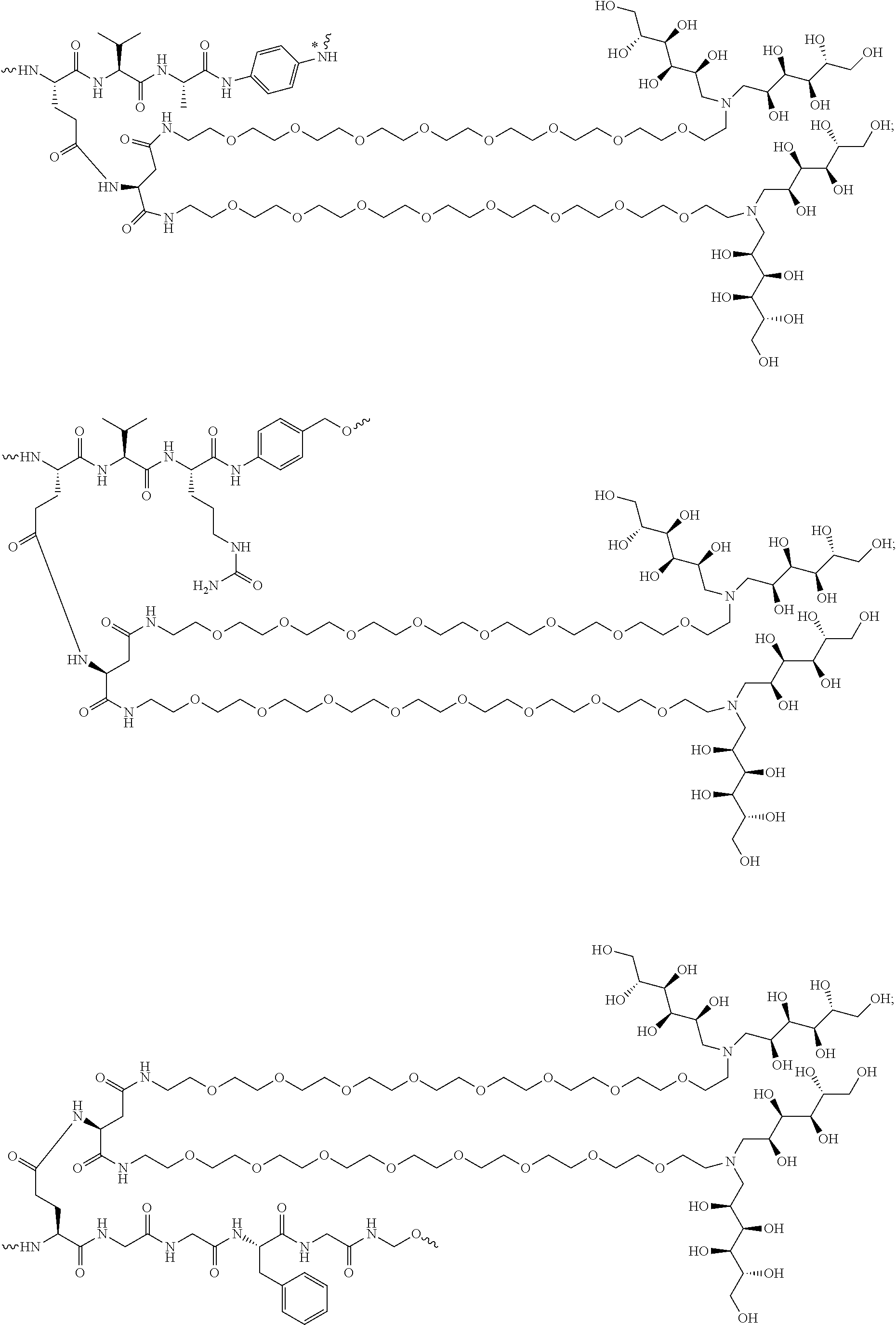 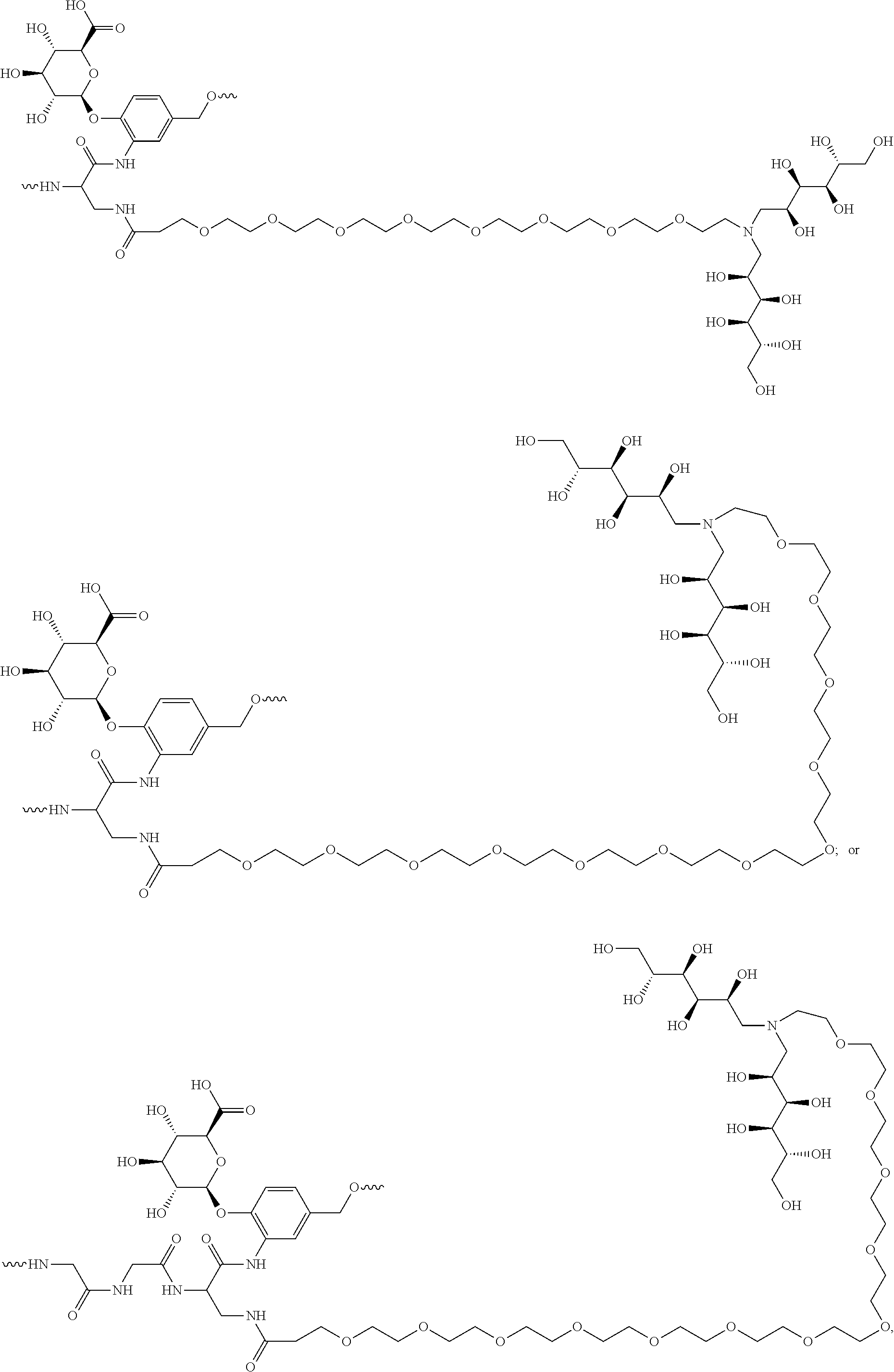
wherein the wavy line [symbol] on the oxygen group or the *-amino group indicates the attachment site to at least one of the Drug units or for a linking group attached to the at least one of the Drug units; and the wavy line [symbol] on the amino group indicates an attachment site for a Stretcher unit or an Amino Acid unit or, prior to attachment, indicates H.

95. The Linker compound of any one of claims 1-94, wherein the Linker unit further comprises a Stretcher unit having an attachment site for a Targeting unit and wherein the Stretcher unit is attached to the Amino Acid unit of the Linker compound.

96. The Linker compound of claim 95, wherein the Stretcher unit is selected from the following: 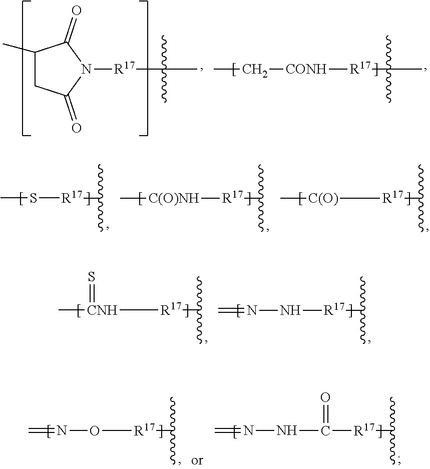
wherein each ( [symbol] ) indicates an attachment site of the Stretcher unit to an Amino Acid unit;
wherein R¹⁷ is —C₁-C₁₀ alkylene-, —C₁-C₁₀ heteroalkylene-, —C₃-C₈ carbocyclo-, —O—(C₁-C₈ alkylene)-, —(CH₂—O—CH₂)_{b}—C₁-C₈ alkylene- (where b is 1 to 26), —C₁-C₈ alkylene-(CH₂—O—CH₂)_{b}— (where b is 1 to 26), —C₁-C₈ alkylene-(CH₂—O—CH₂)_{b}—C₁-C₈alkylene- (where b is 1 to 26), -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₁-C₈ alkylene-(CH₂—O—CH₂)_{b}—C(═O)— (where b is 1 to 26), —(CH₂—O—CH₂)_{b}—C₁-C₈ alkylene-C(═O)— (where b is 1 to 26), —C₁-C₈ alkylene-(CH₂—O—CH₂)_{b}—C₁-C₈ alkylene-C(═O)— (where b is 1 to 26), —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₁-C₈ alkylene-(CH₂—O—CH₂)_{b}—NH— (where b is 1 to 26), —(CH₂—O—CH₂)_{b}—C₁-C₈ alkylene-NH— (where b is 1 to 26), —C₁-C₈ alkylene-(CH₂—O—CH₂)_{b}—C₁-C₈ alkylene-NH— (where b is 1 to 26), —C₁-C₈ alkylene-(C(═O))—NH—(CH₂—O—CH₂)_{b}—C(═O)— (where b is 1 to 26), —C₁—C₈ alkylene-(C(═O))—NH—(CH₂—O—CH₂)_{b}—C₁-C₈ alkylene-C(═O)— (where b is 1 to 26), —C₁-C₈ alkylene-NH—(C(═O))—(CH₂—O—CH₂)_{b}—NH— (where b is 1 to 26), —C₁-C₈ alkylene-NH—(C(═O))—(CH₂—O—CH₂)_{b}—C₁-C₈ alkylene-NH— (where b is 1 to 26), —C₃-C₈ carbocyclo-NH—, —O—(C₁-C₈ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkyl)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—; or
wherein the Stretcher unit comprises maleimido(C₁-C₁₀alkylene-C(O)—, maleimido(CH₂OCH₂)ₚ₂(C₁-C₁₀alkylene)C(O)—, maleimido(C₁-C₁₀alkylene)(CH₂OCH₂)ₚ₂C(O)—, or a ring open form thereof, wherein p2 is from 1 to 26.

97. The Linker compound of claim 95, wherein the Stretcher unit is selected from the following: 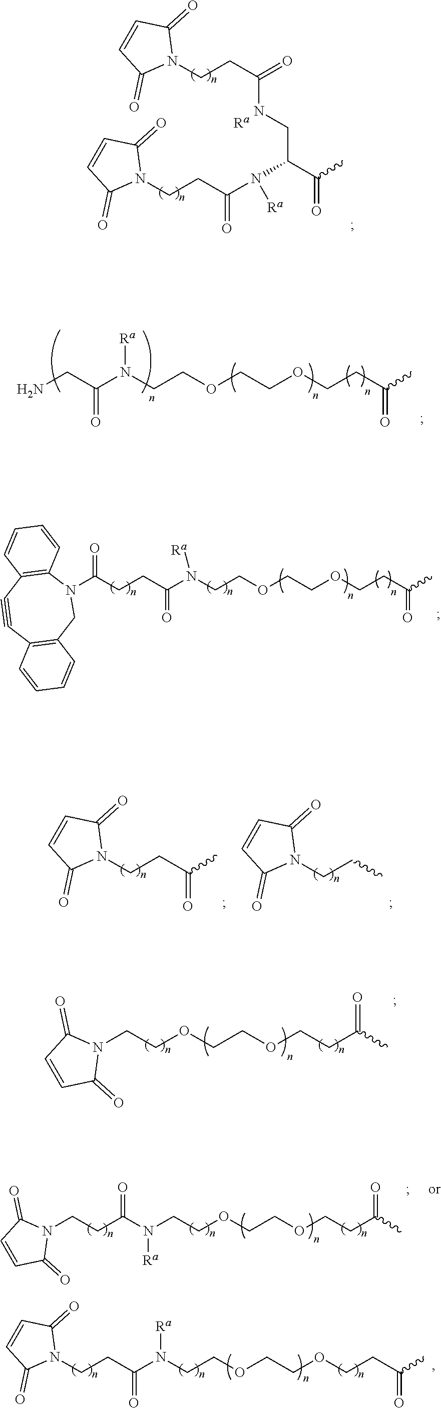
or a stereoisomer thereof wherein each R^{a} is independently H or C₁₋₆ alkyl, each n is independently 0-12, and the wavy line [symbol] indicates an attachment site of the Stretcher unit to the Amino Acid unit, and the attachment site for the Targeting unit is on a maleimide, primary amine or alkyne functional group.

98. The Linker compound or Linker of claim 95, wherein the Stretcher unit is selected from the following: 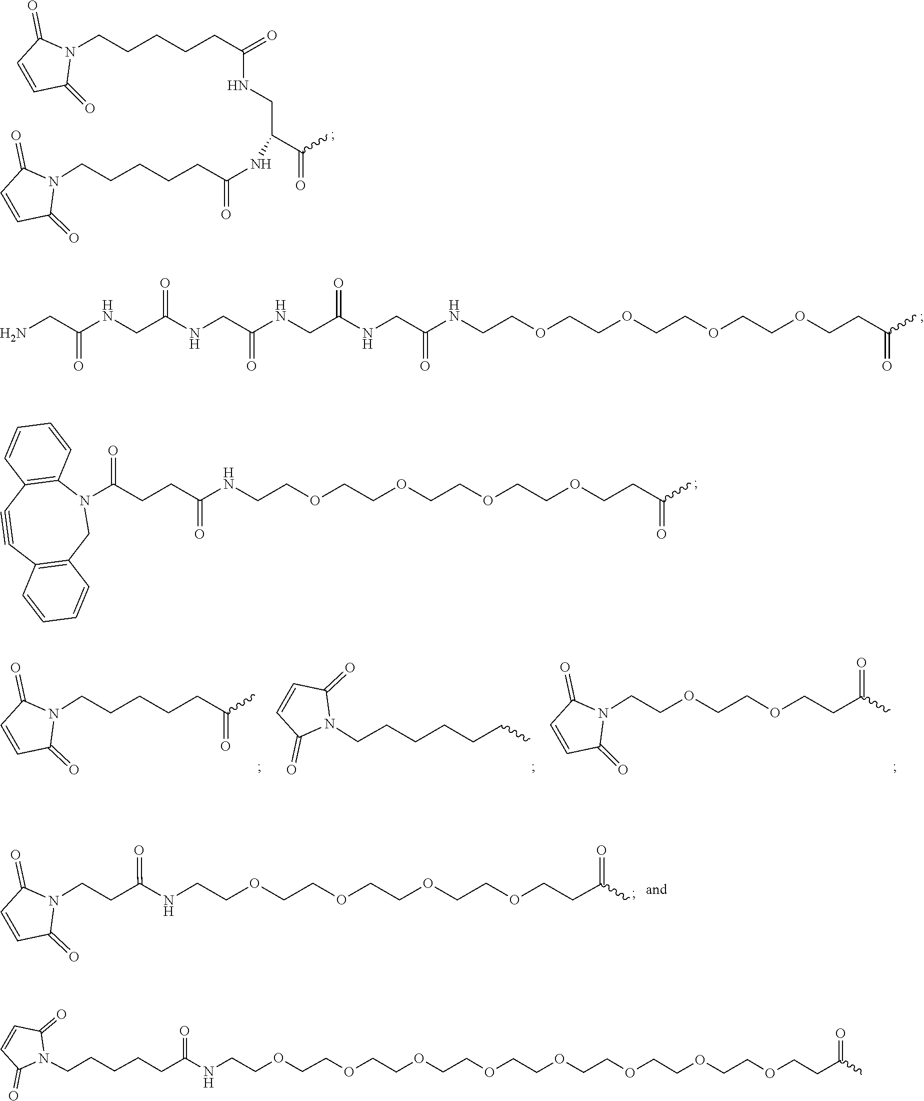
or a stereoisomer thereof, wherein the wavy line [symbol] indicates an attachment site of the Stretcher unit to an Amino Acid unit, and the attachment site for the Targeting unit is on a maleimide, primary amine or alkyne functional group.

99. The Linker compound of any one of claims 1-98, comprising one of the following structures: 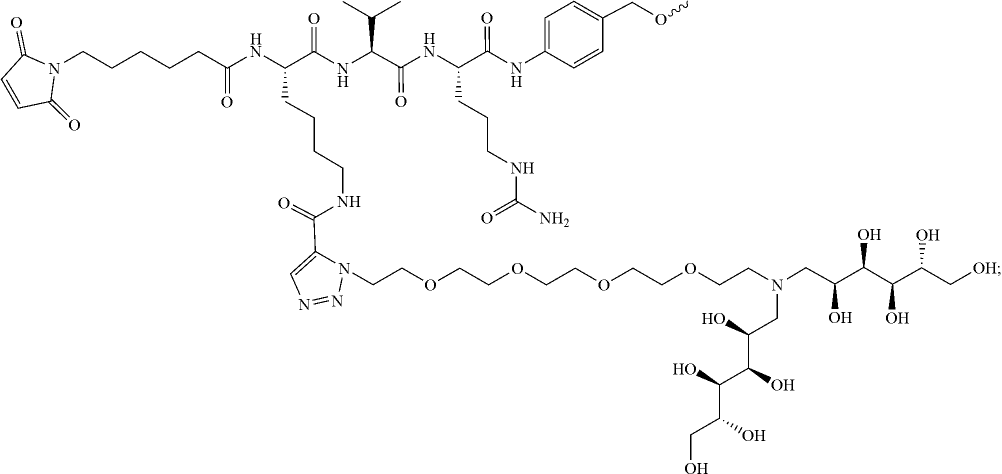 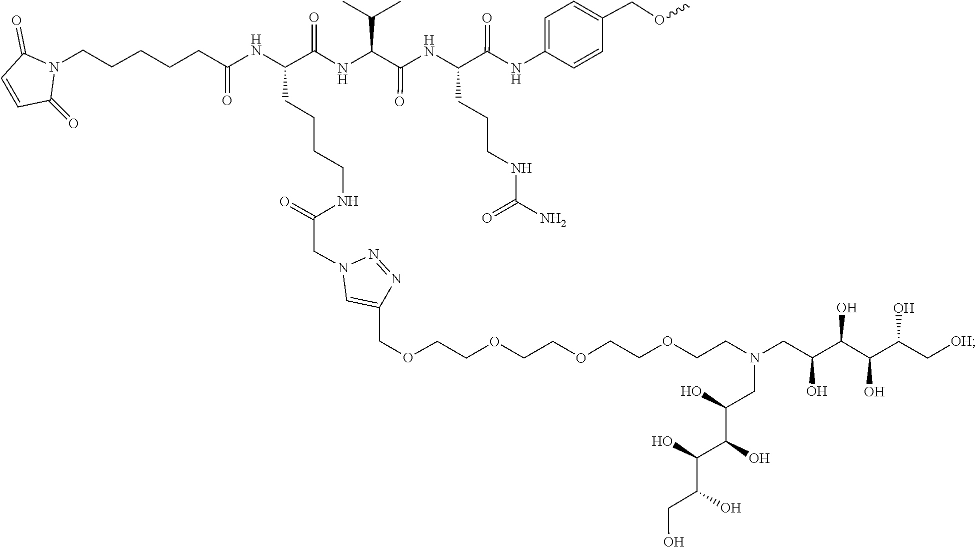 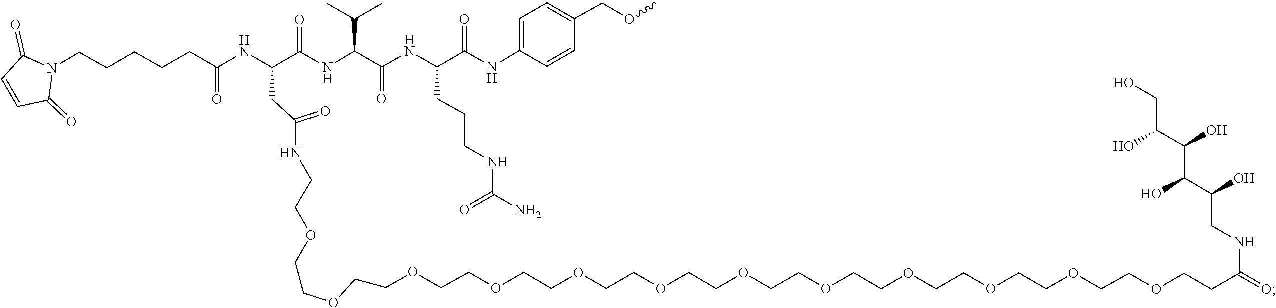 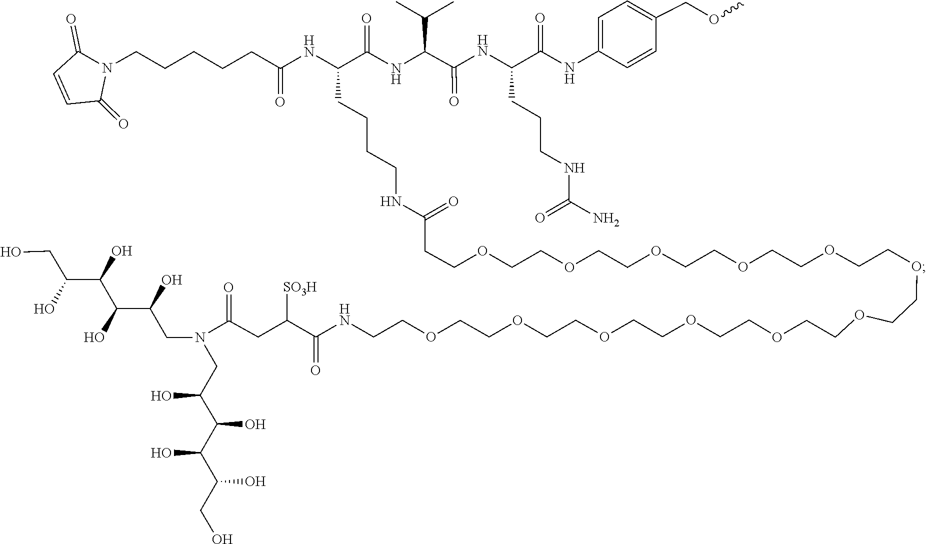 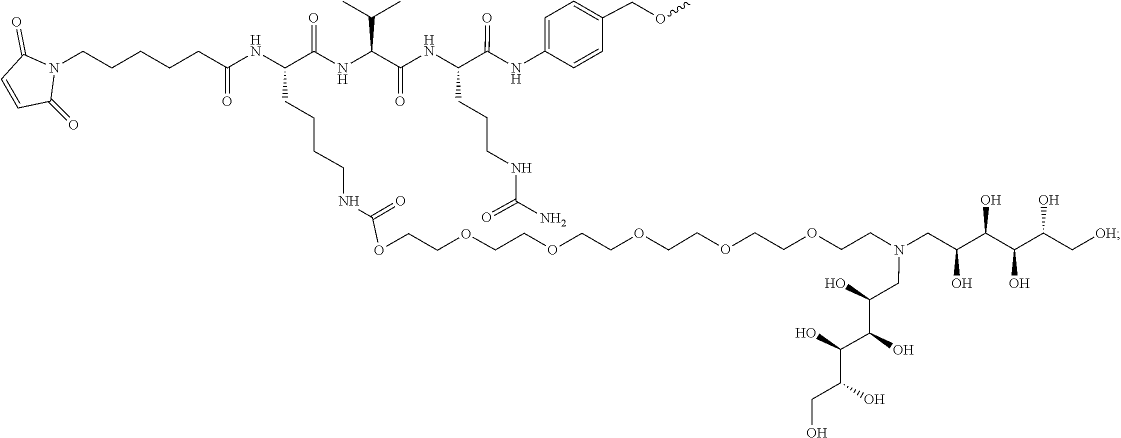 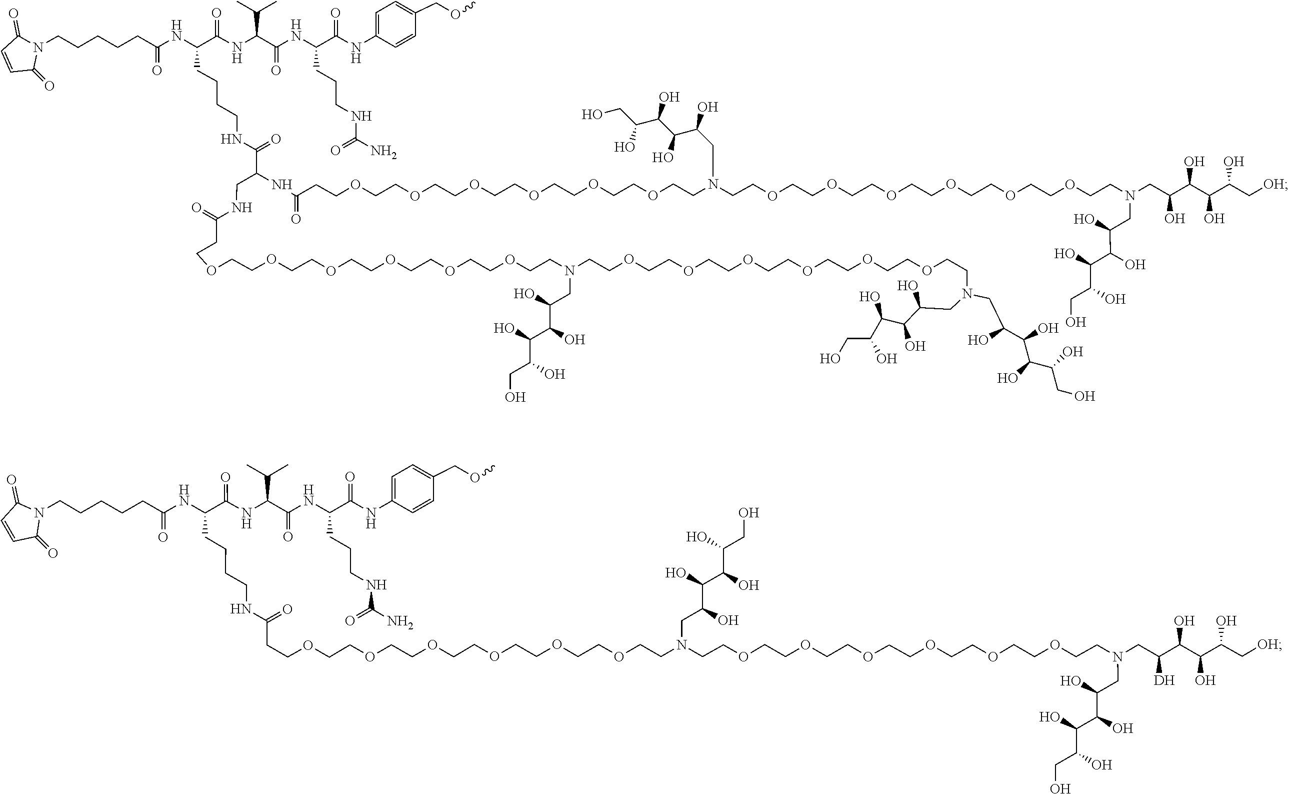 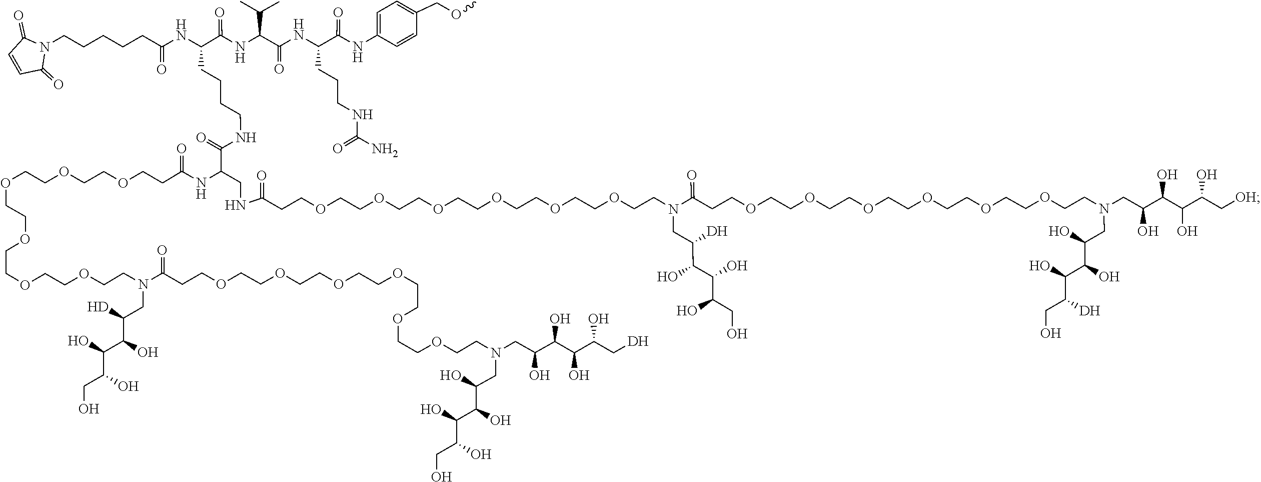 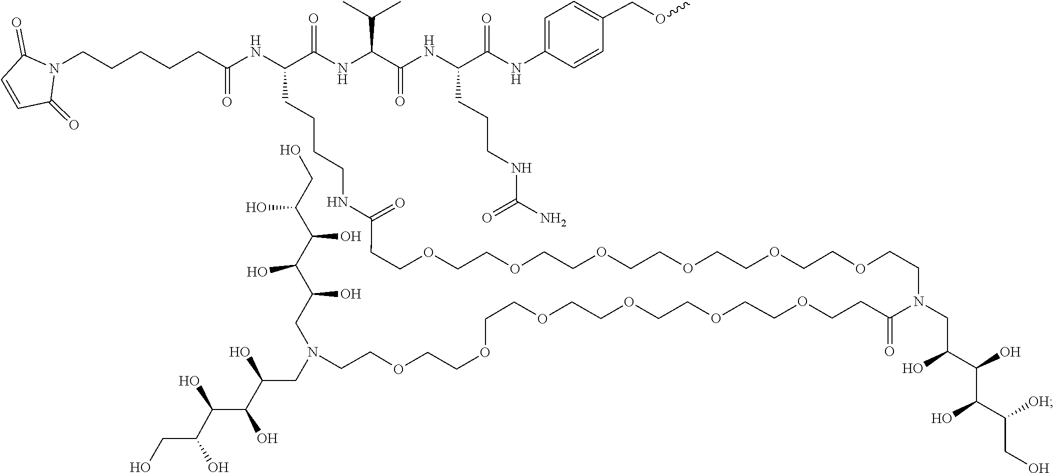 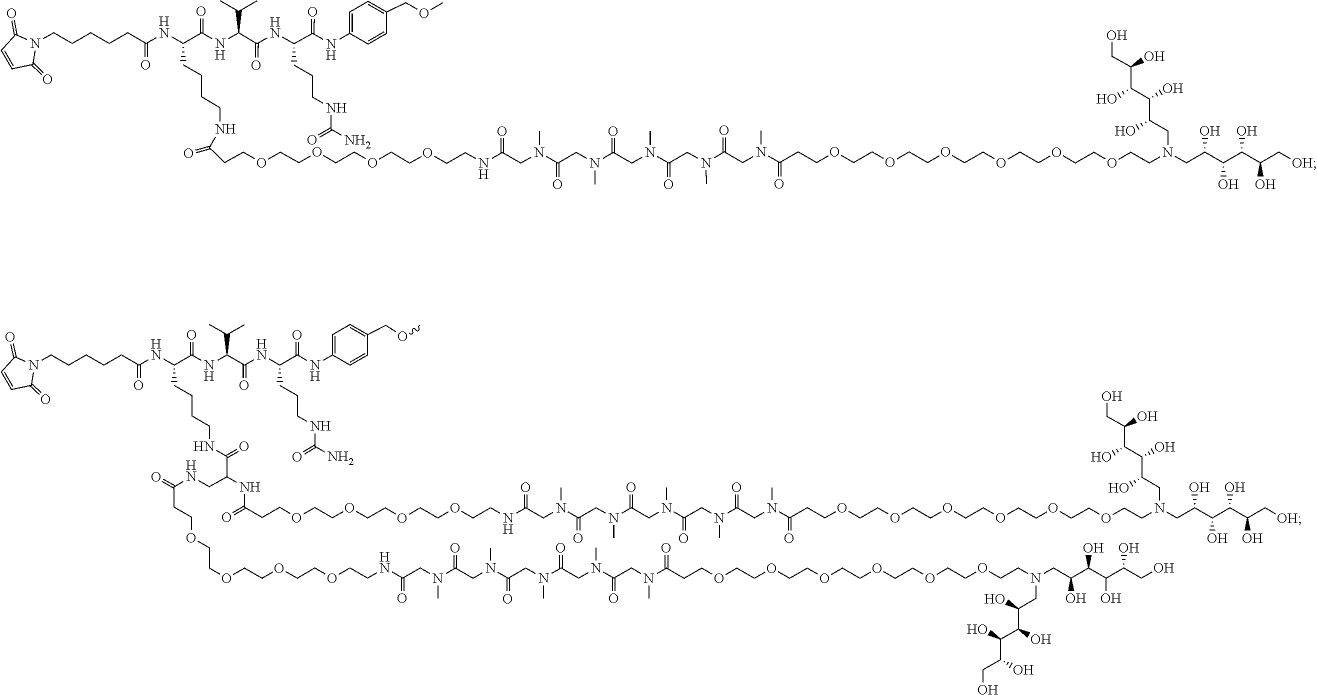 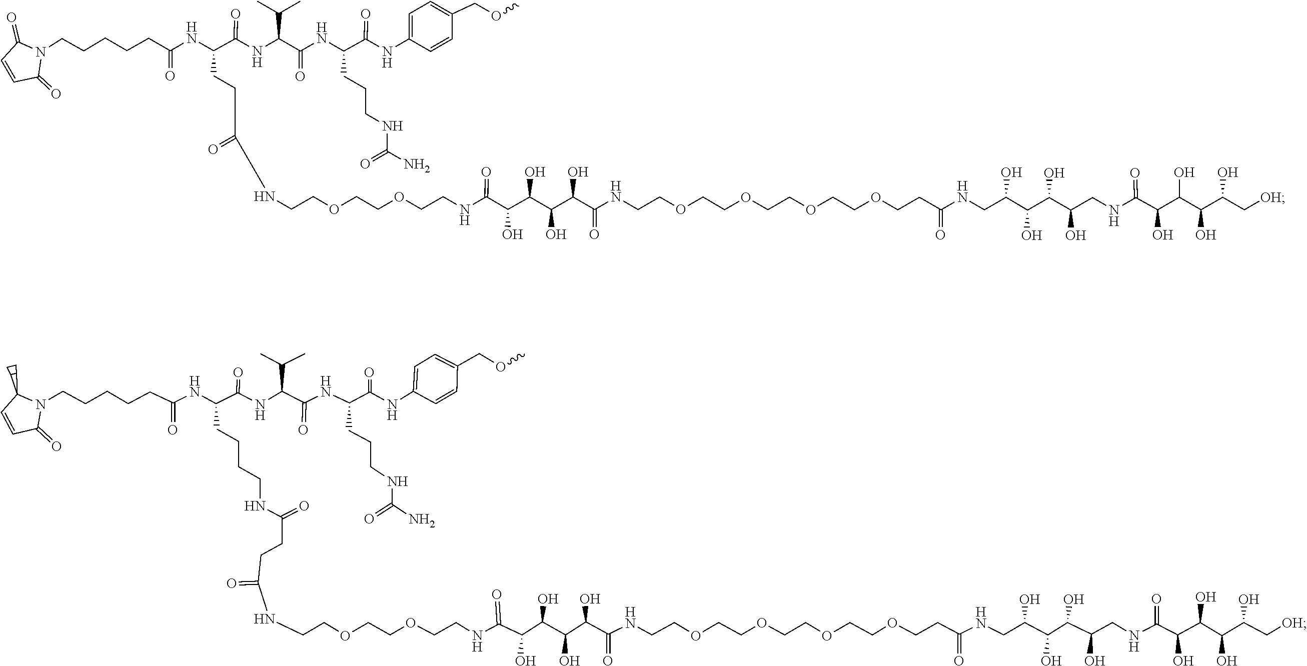 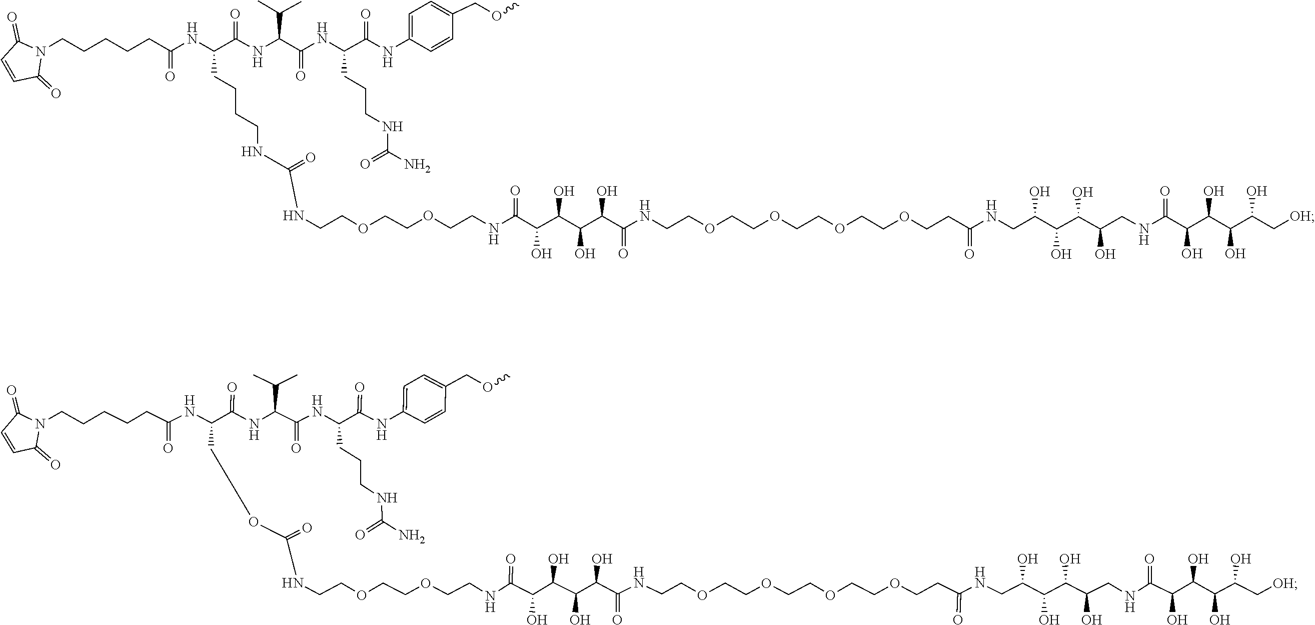 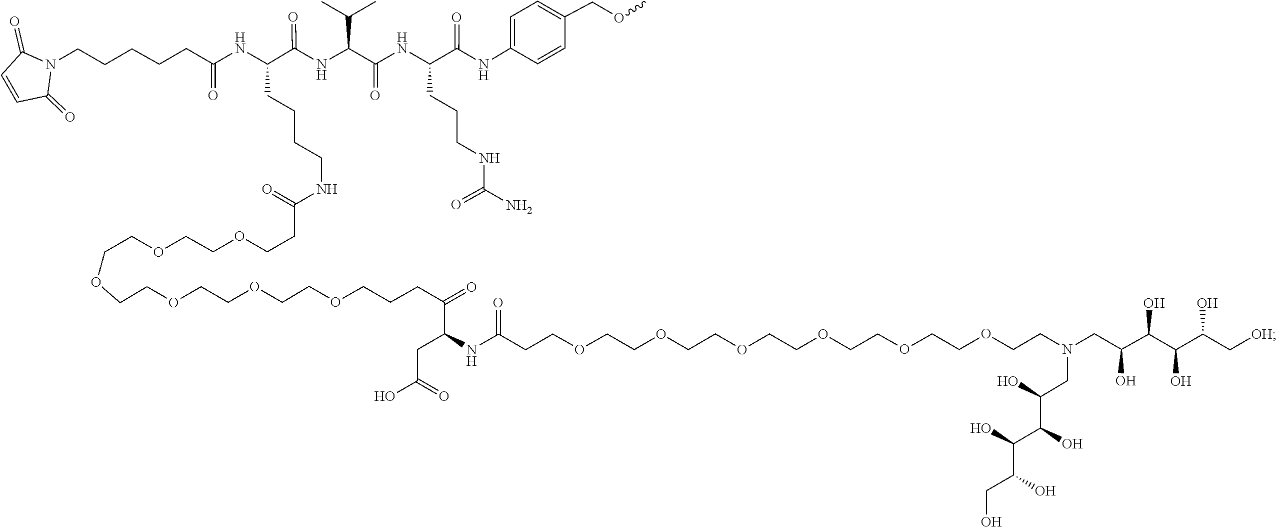 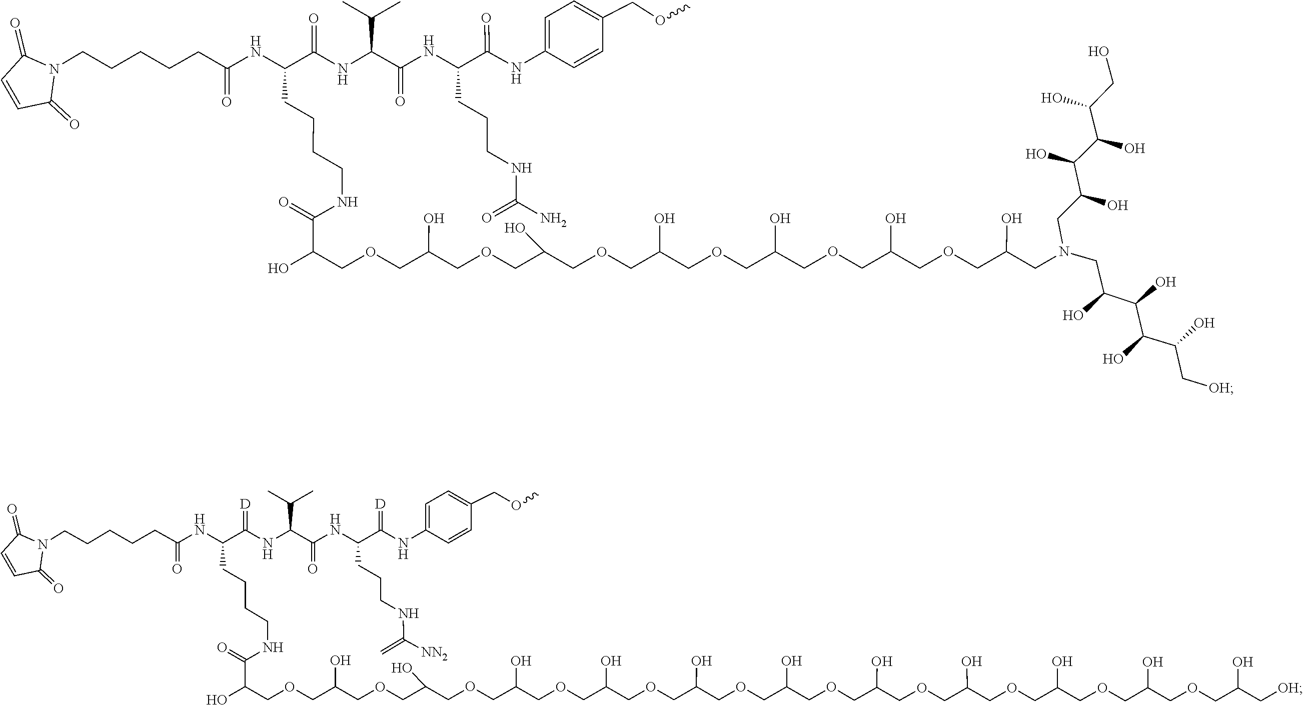 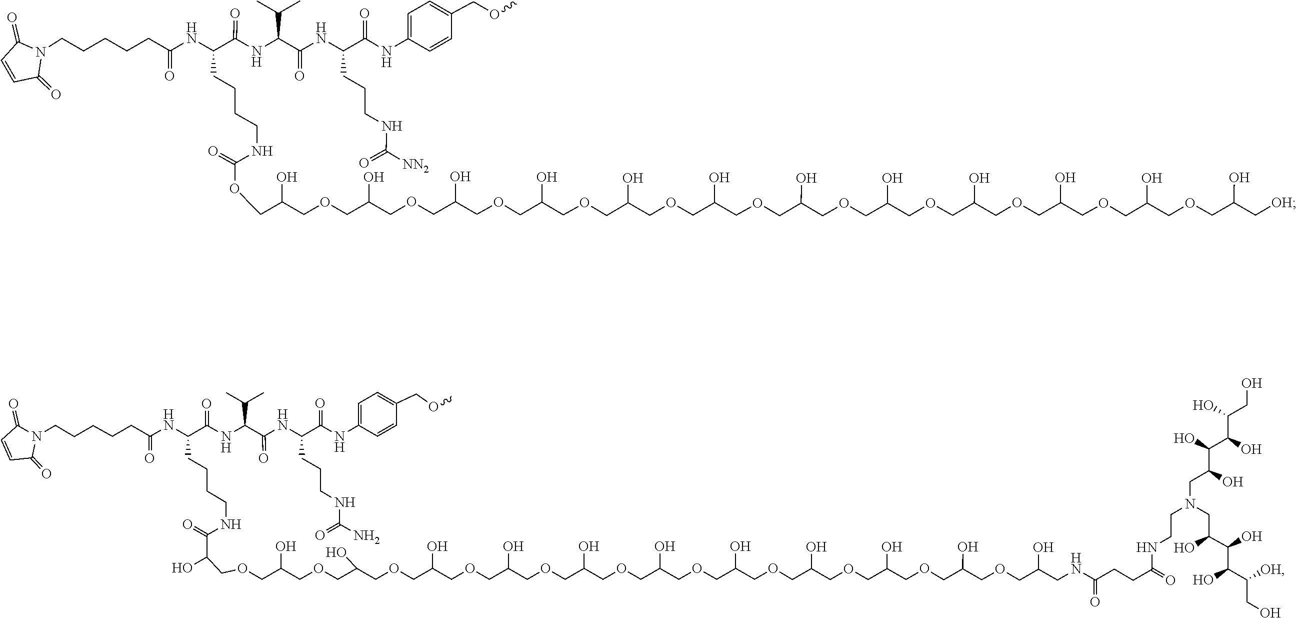 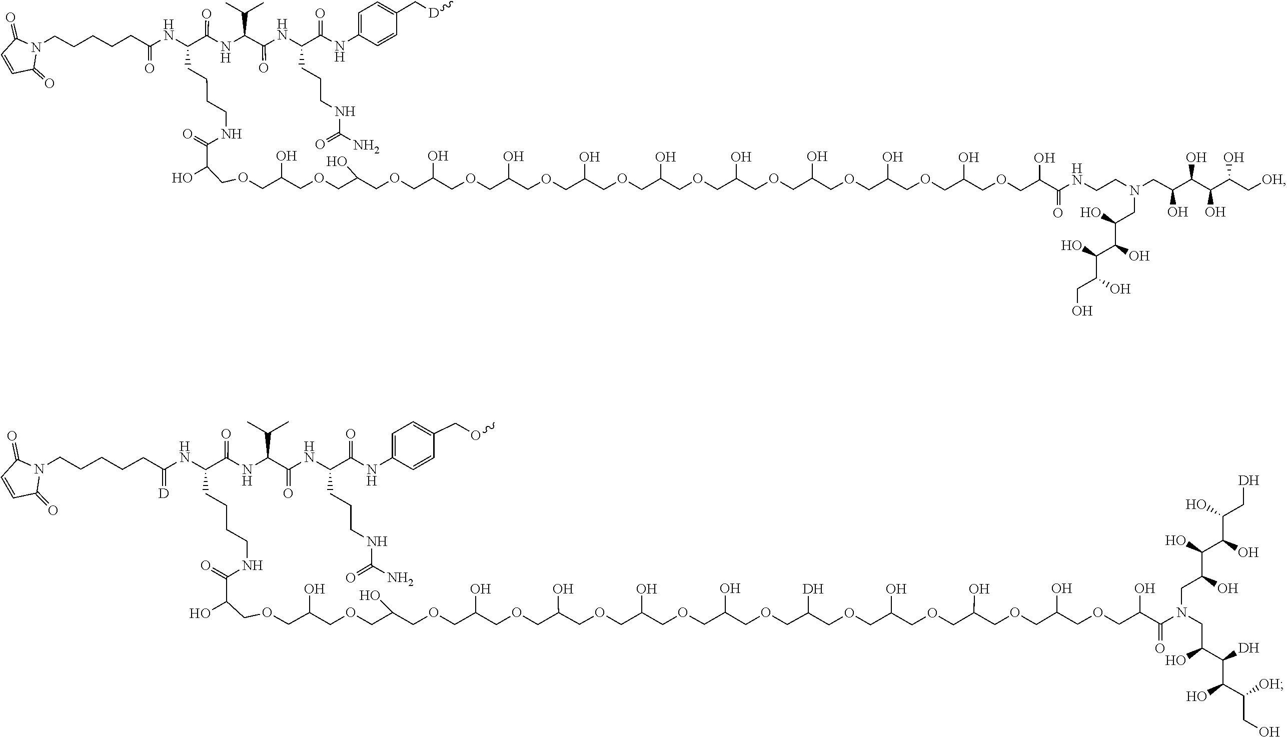 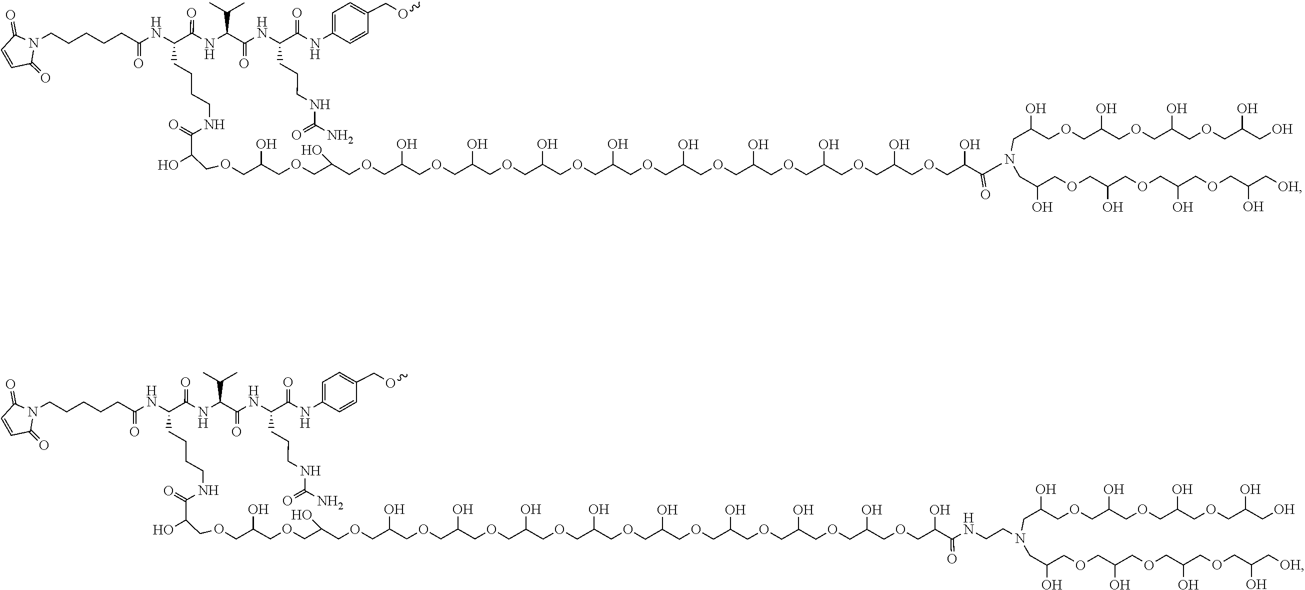 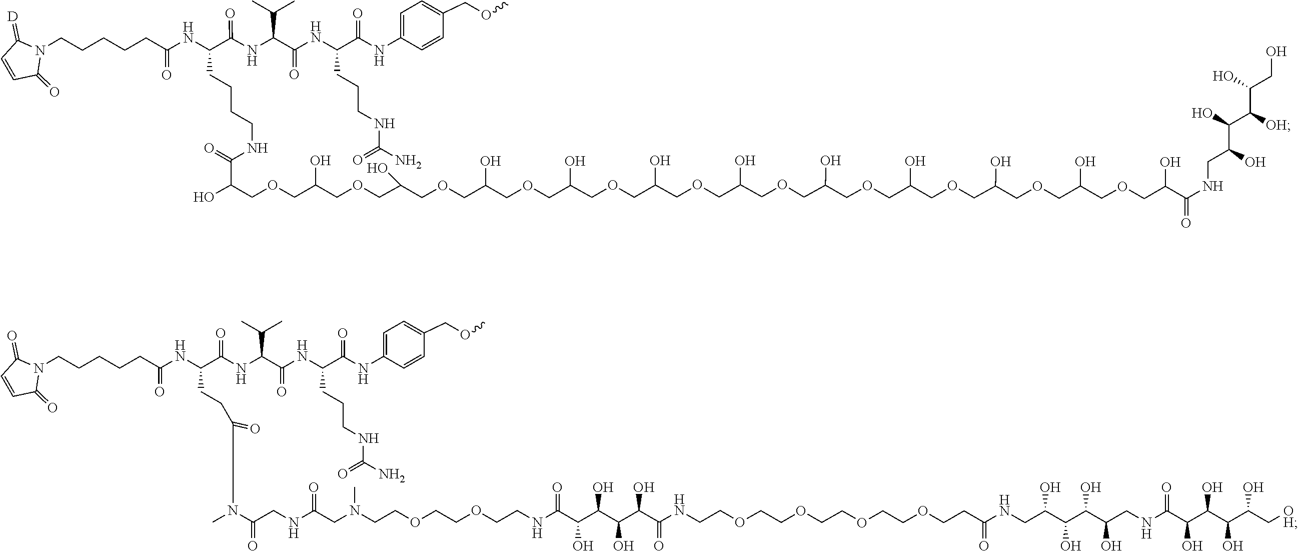 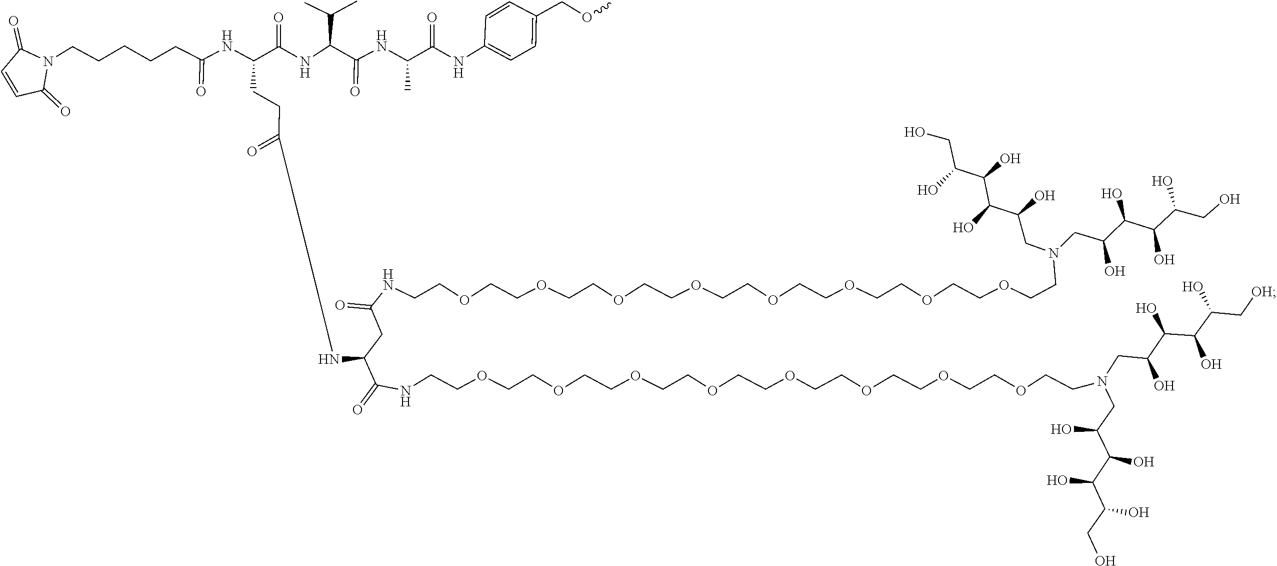 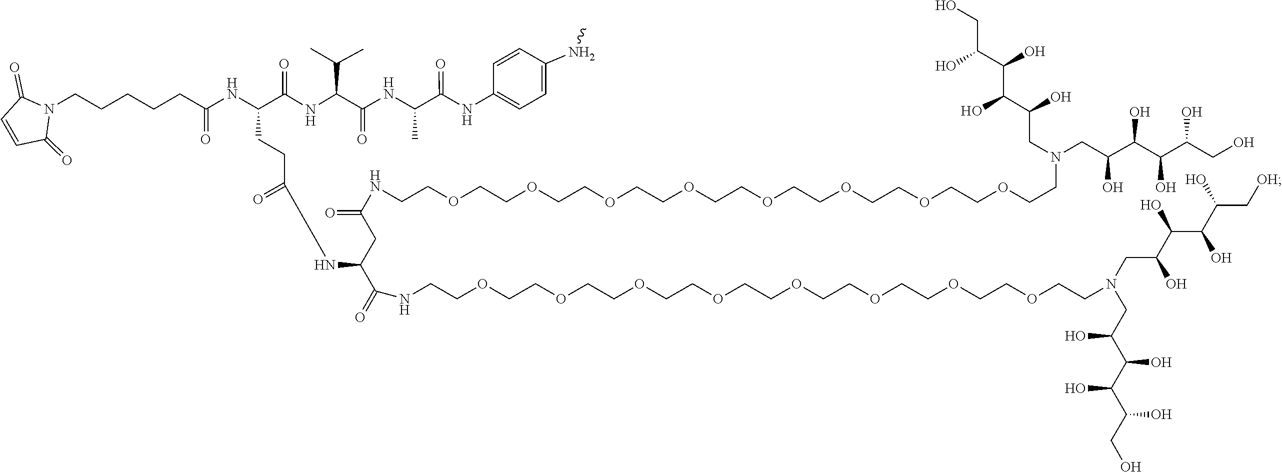 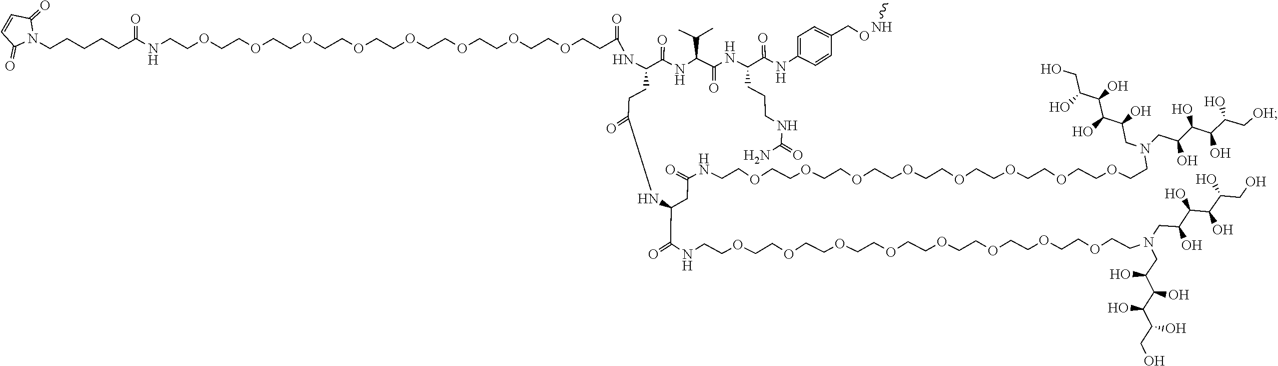 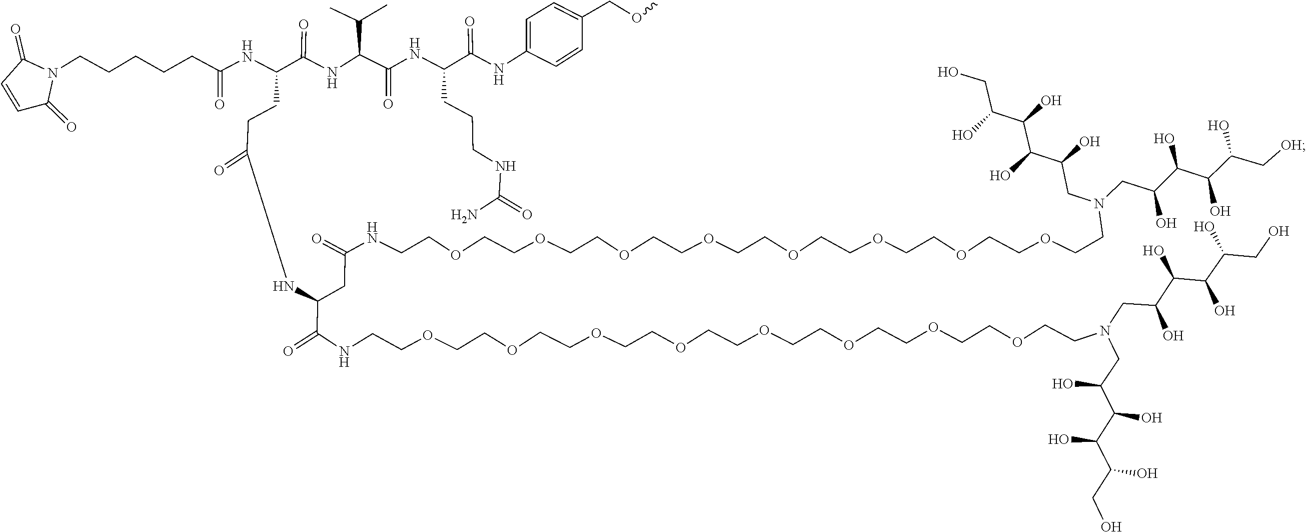 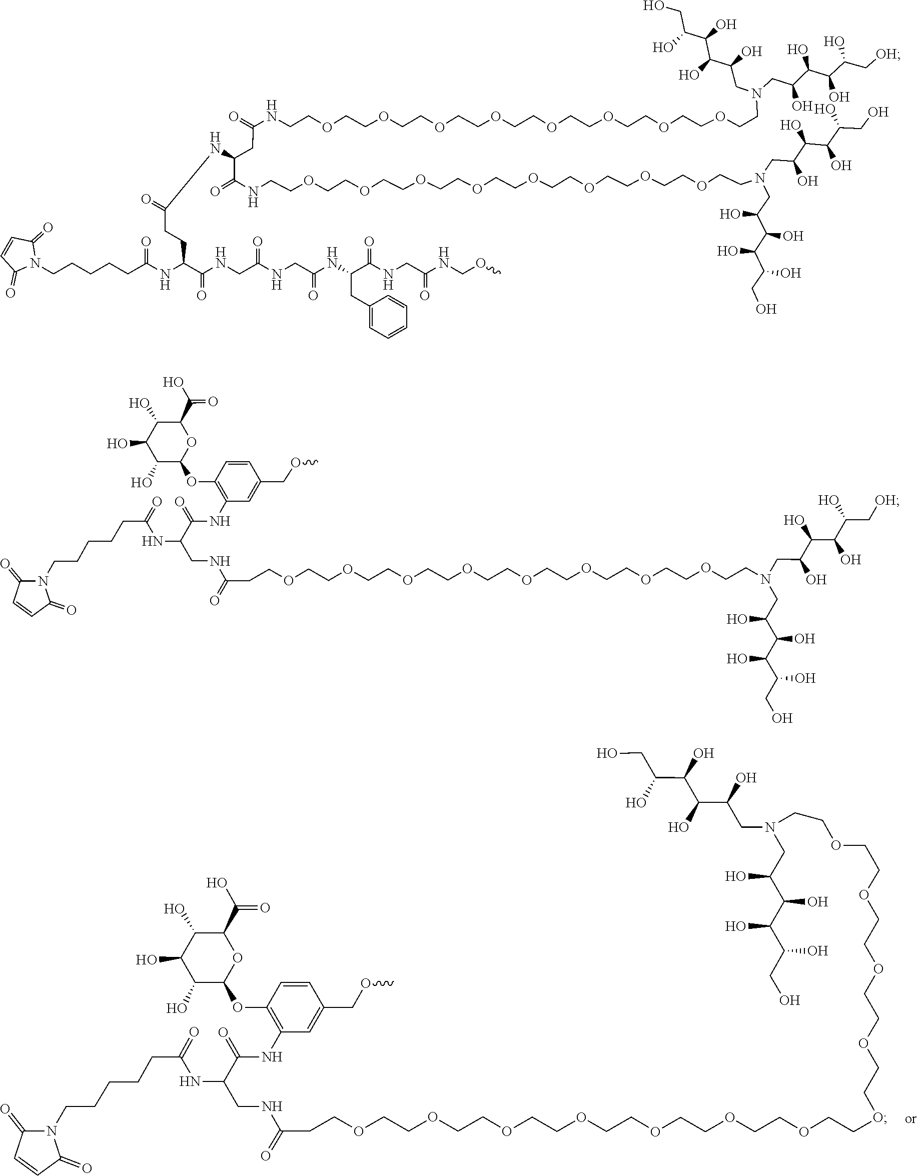 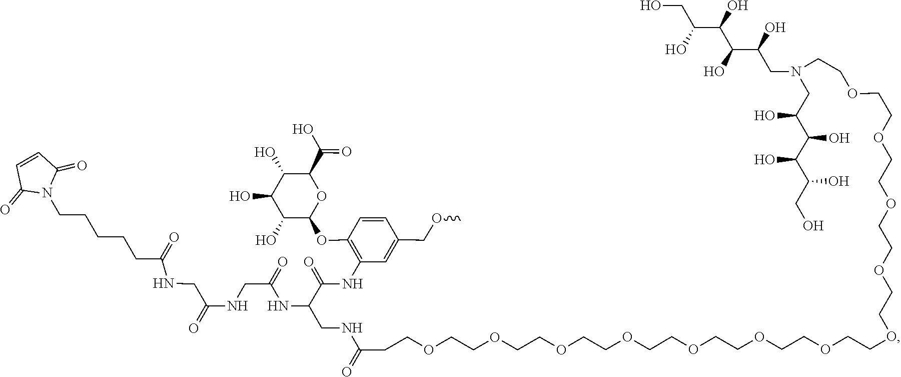
or a stereoisomer thereof, wherein the wavy line [symbol] indicates the attachment site to at least one of the Drug units or for a linking group attached to the at least one of the Drug units.

100. A Drug-Linker compound, comprising a Linker compound of any one of claims 1-99 conjugated to at least one Drug unit.

101. The Drug-Linker of claim 100, wherein the Drug unit is selected from a cytotoxic agent, an immune modulatory agent, a nucleic acid, a growth inhibitory agent, a PROTAC, a toxin, a radioactive isotope and a chelating ligand.

102. The Drug-Linker of claim 101, wherein the Drug unit is a cytotoxic agent.

103. The Drug-Linker of claim 102, wherein the cytotoxic agent is selected from the group consisting of an auristatin, a maytansinoid, a camptothecin, a duocarmycin, and a calicheamicin.

104. The Drug-Linker of claim 103, wherein the cytotoxic agent is an auristatin.

105. The Drug-Linker of claim 104, wherein the cytotoxic agent is MMAE or MMAF.

106. The Drug-Linker of claim 103, wherein the cytotoxic agent is a camptothecin.

107. The Drug-Linker of claim 106, wherein the cytotoxic agent is exatecan or SN-38.

108. The Drug-Linker of claim 107, wherein the cytotoxic agent is RS-exatecan or SS-exatecan.

109. The Drug-Linker of claim 103, wherein the cytotoxic agent is a calicheamicin.

110. The Drug-Linker of claim 103, wherein the cytotoxic agent is a maytansinoid.

111. The Drug-Linker of claim 110, wherein the maytansinoid is maytansine, maytansinol or ansamatocin-2.

112. The Drug-Linker of claim 101, wherein the Drug unit is an immune modulatory agent.

113. The Drug-Linker of claim 112, wherein the immune modulatory agent is selected from a TRL7 agonist, a TLR8 agonist, a STING agonist, or a RIG-I agonist.

114. The Drug-Linker of claim 113, wherein the immune modulatory agent is an TLR7 agonist.

115. The Drug-Linker of claim 114, wherein the TLR7 agonist is an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, or PolyG3.

116. The Drug-Linker of claim 113, wherein the immune modulatory agent is a TLR8 agonist.

117. The Drug-Linker of claim 116, wherein the TLR8 agonist is selected from an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA.

118. The Drug-Linker of claim 113, wherein the immune modulatory agent is a STING agonist.

119. The Drug-Linker of claim 113, wherein the immune modulatory agent is a RIG-I agonist.

120. The Drug-Linker of claim 119, wherein the RIG-I agonist is selected from KIN1148, SB-9200, KIN700, KIN600, KIN500, KIN100, KIN101, KIN400 and KIN2000.

121. The Drug-Linker of claim 101, wherein the Drug unit is a chelating ligand.

122. The Drug-Linker of claim 121, wherein the chelating ligand is selected from platinum (Pt), ruthenium (Ru), rhodium (Rh), gold (Au), silver (Ag), copper (Cu), molybdenum (Mo), titanium (Ti), or iridum (Ir); a radioisotope such as yttrium-88, yttrium-90, technetium-99, copper-67, rhenium-188, rhenium-186, gallium-66, gallium-67, indium-111, indium-114, indium-115, lutetium-177, strontium-89, sararium-153, and lead-212.

123. The Drug-Linker of claim 100, having one of the following structures: 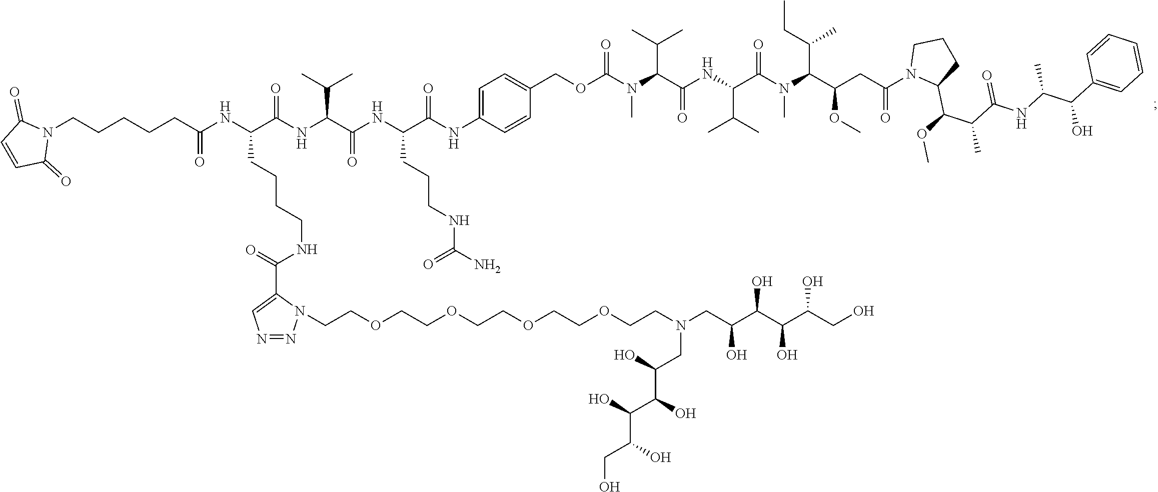 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCCNC(=O)c2cnnn2CCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN2C(=O)C=CC2=O)C(C)C)cc1)C(C)C] 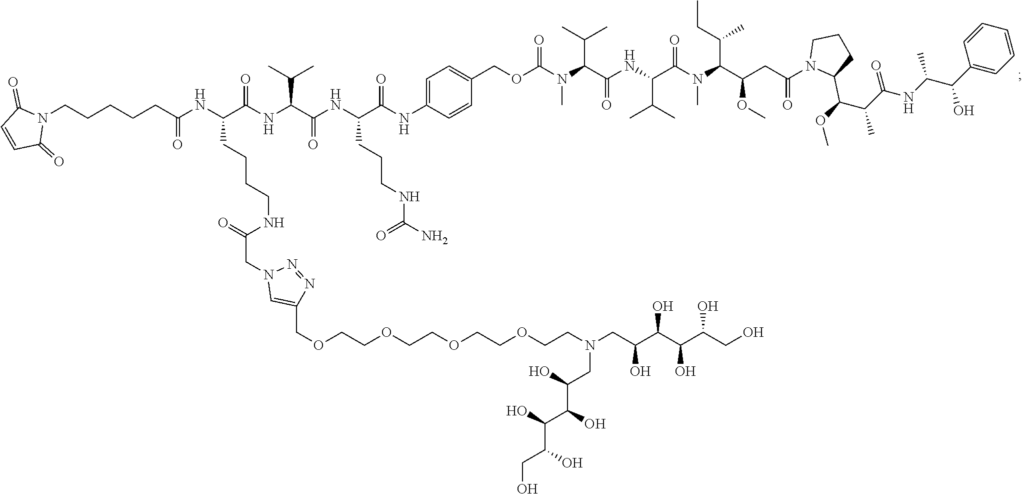 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCCNC(=O)Cn2cc(COCCOCCOCCOCCN(C[C@H](O)[C@@H](O)[C@H](O)[C@H](O)CO)C[C@H](O)[C@@H](O)[C@H](O)[C@H](O)CO)nn2)NC(=O)CCCCCN2C(=O)C=CC2=O)C(C)C)cc1)C(C)C] 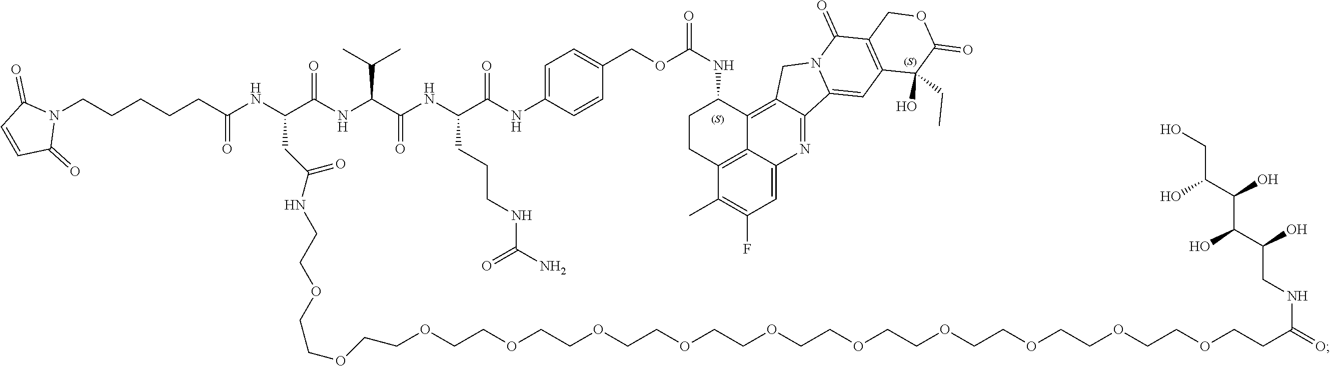 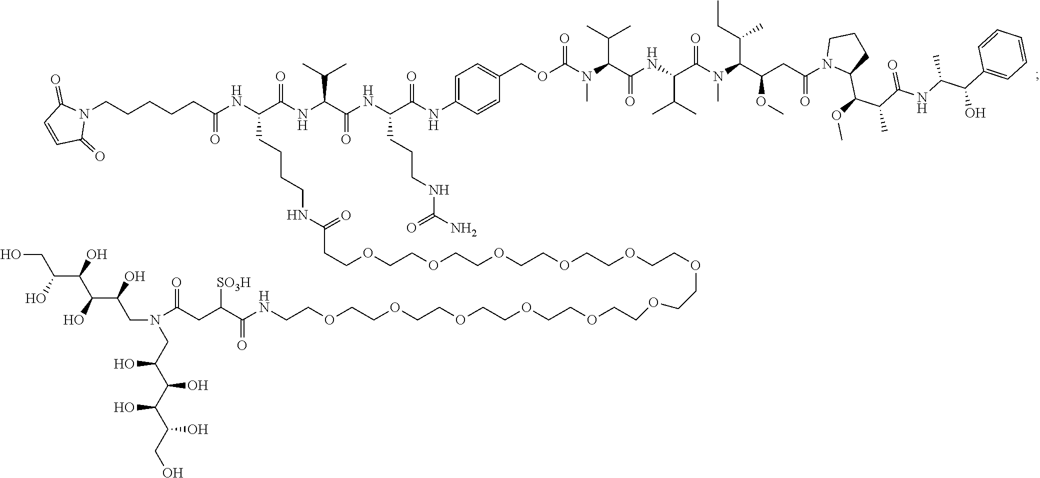 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOCCNC(=O)C(CC(=O)N(C[C@H](O)[C@@H](O)[C@H](O)[C@H](O)CO)C[C@H](O)[C@@H](O)[C@H](O)[C@H](O)CO)S(=O)(=O)O)NC(=O)CCCCCN2C(=O)C=CC2=O)C(C)C)cc1)C(C)C] 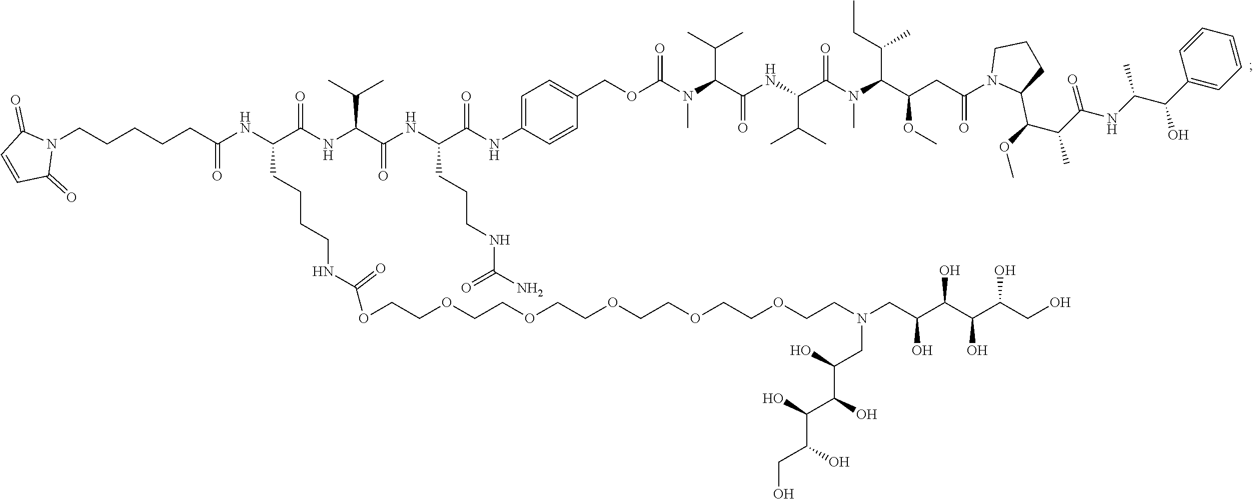 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCCNC(=O)OCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN2C(=O)C=CC2=O)C(C)C)cc1)C(C)C] 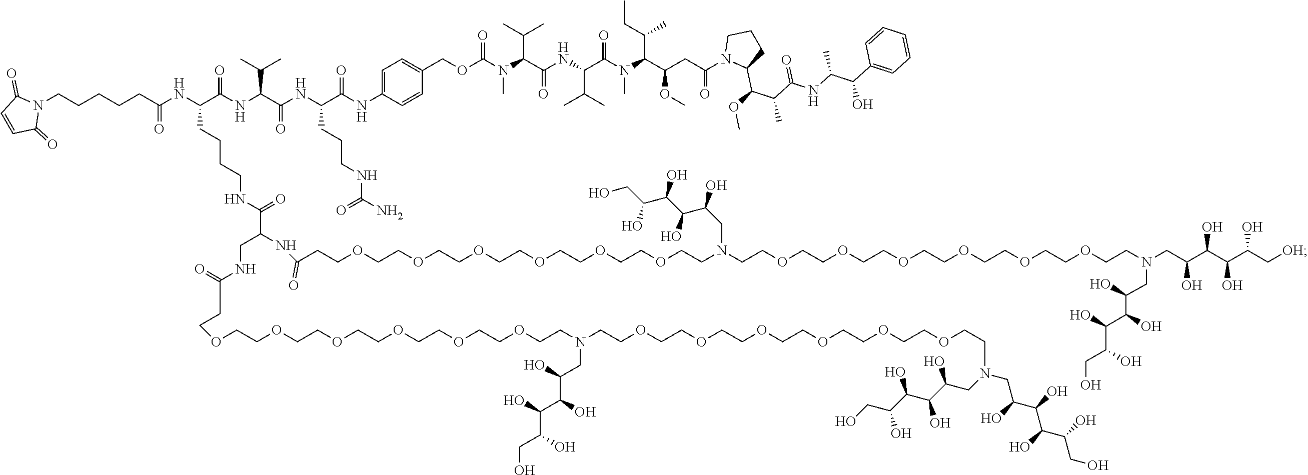 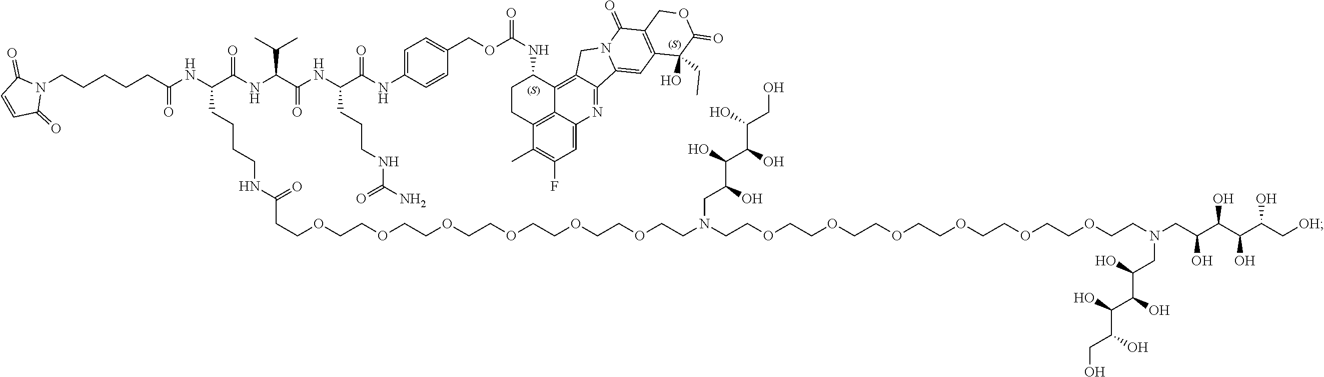 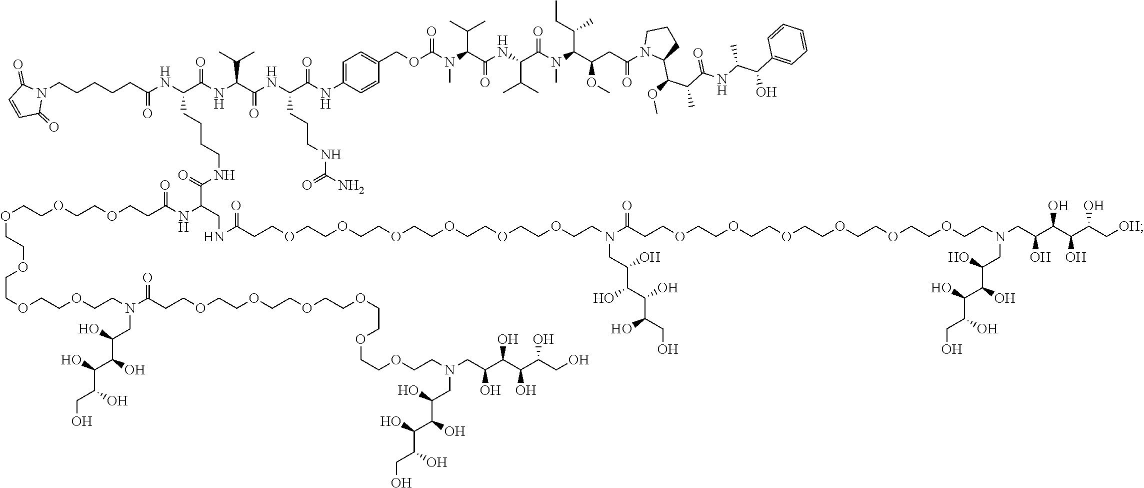 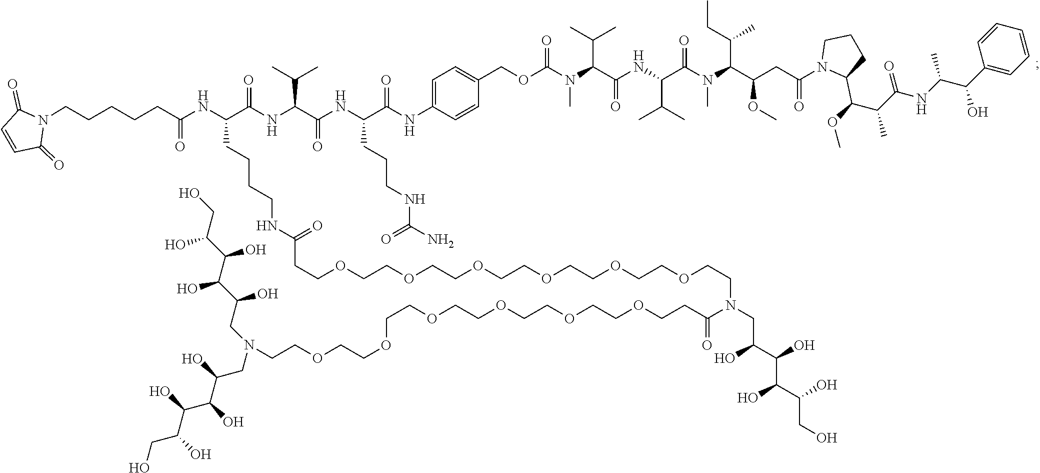 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCCNC(=O)CCOCCOCCOCCOCCOCCOCCN(C[C@H](O)[C@@H](O)[C@H](O)[C@H](O)CO)C(=O)CCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN2C(=O)C=CC2=O)C(C)C)cc1)C(C)C] 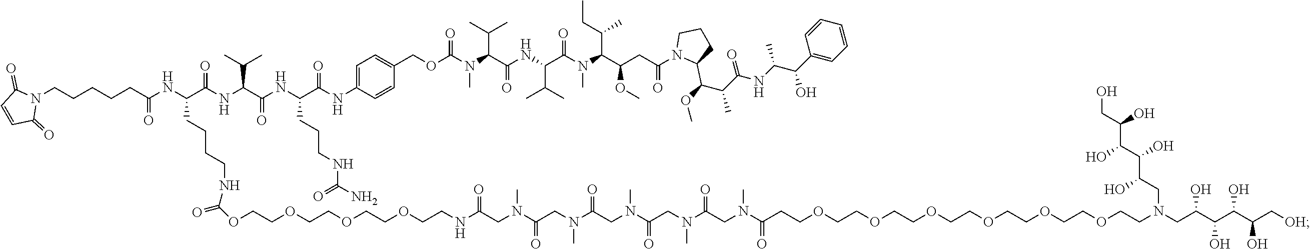 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCCNC(=O)OCCOCCOCCOCCNC(=O)CN(C)C(=O)CN(C)C(=O)CN(C)C(=O)CN(C)C(=O)CN(C)C(=O)CCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN2C(=O)C=CC2=O)C(C)C)cc1)C(C)C] 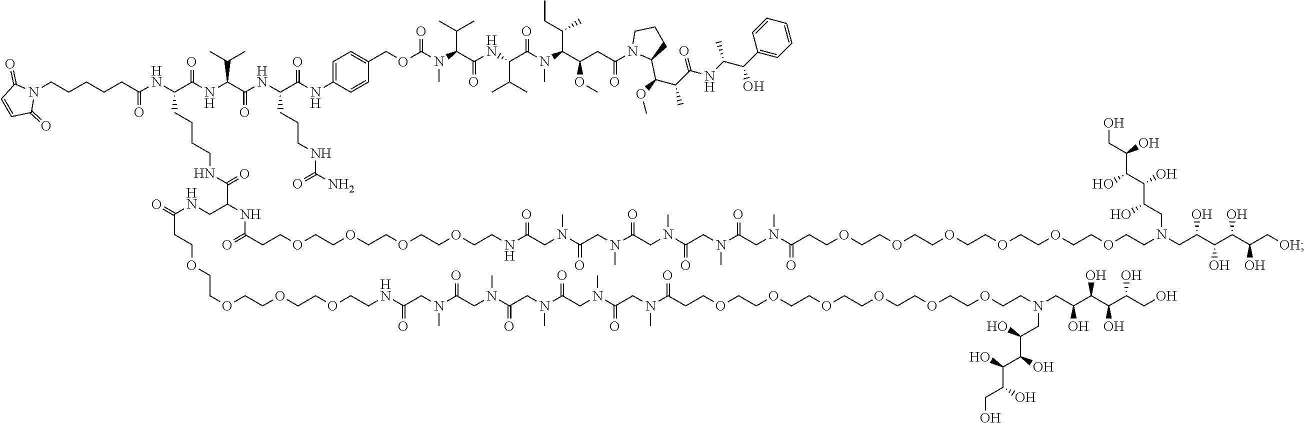 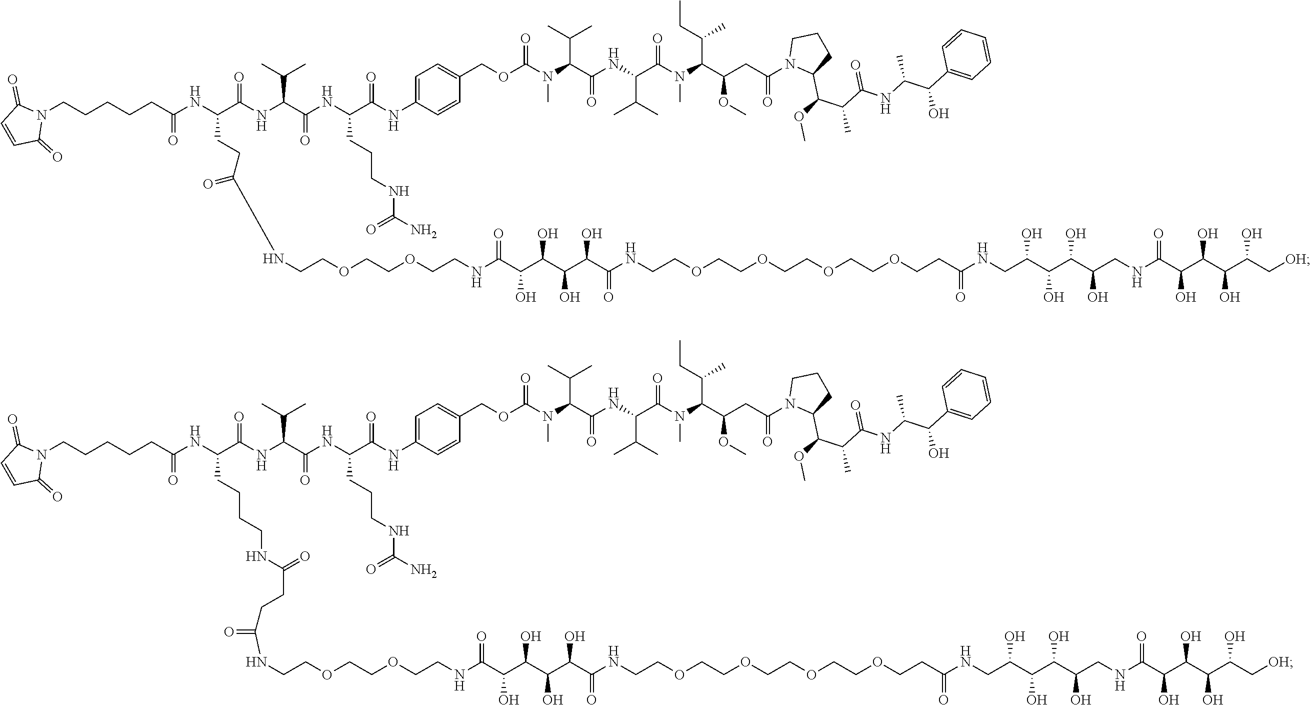 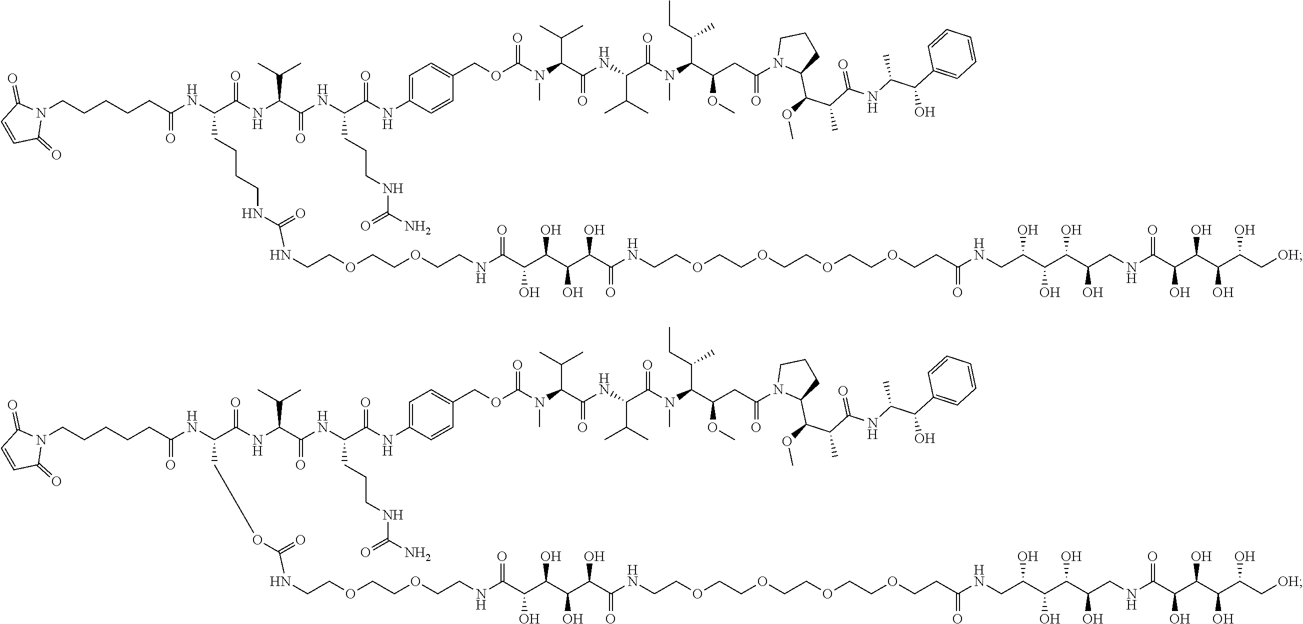 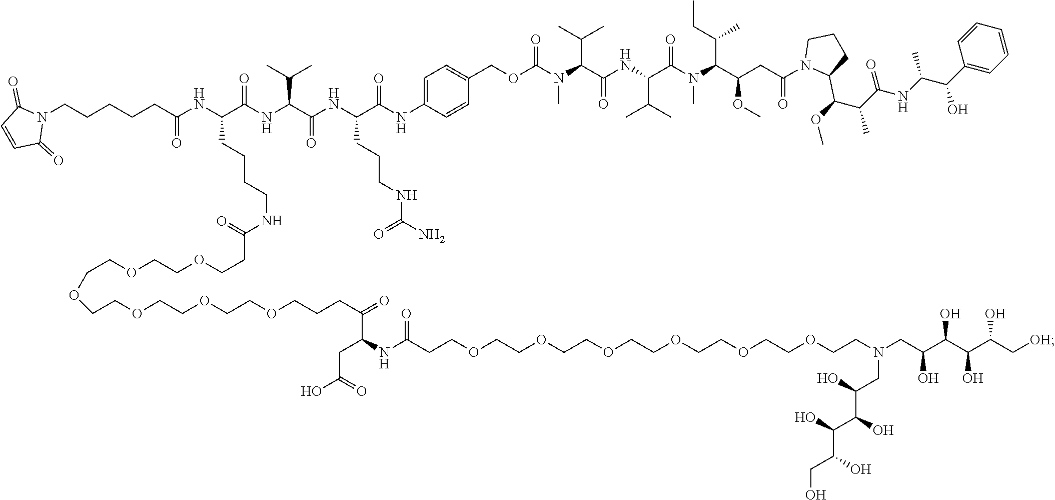 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCCNC(=O)CCOCCOCCOCCOCCOCCOCCCC(=O)[C@H](CC(=O)O)NC(=O)CCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN2C(=O)C=CC2=O)C(C)C)cc1)C(C)C] 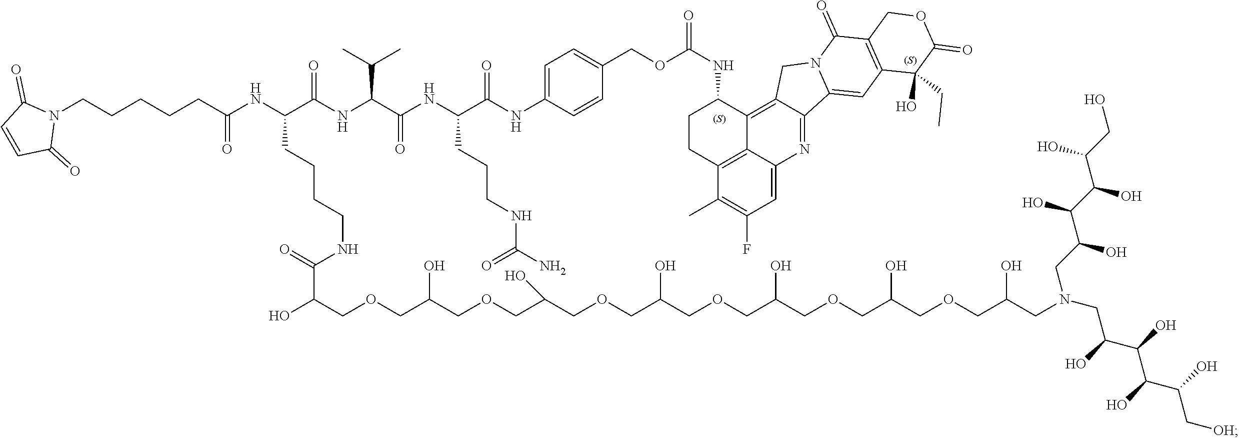 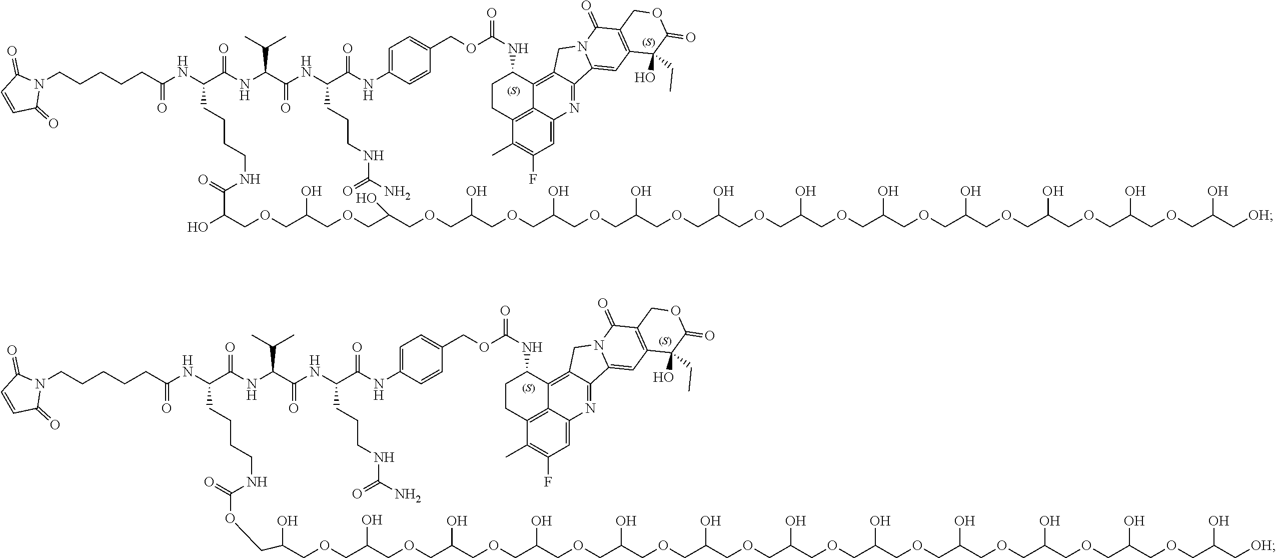 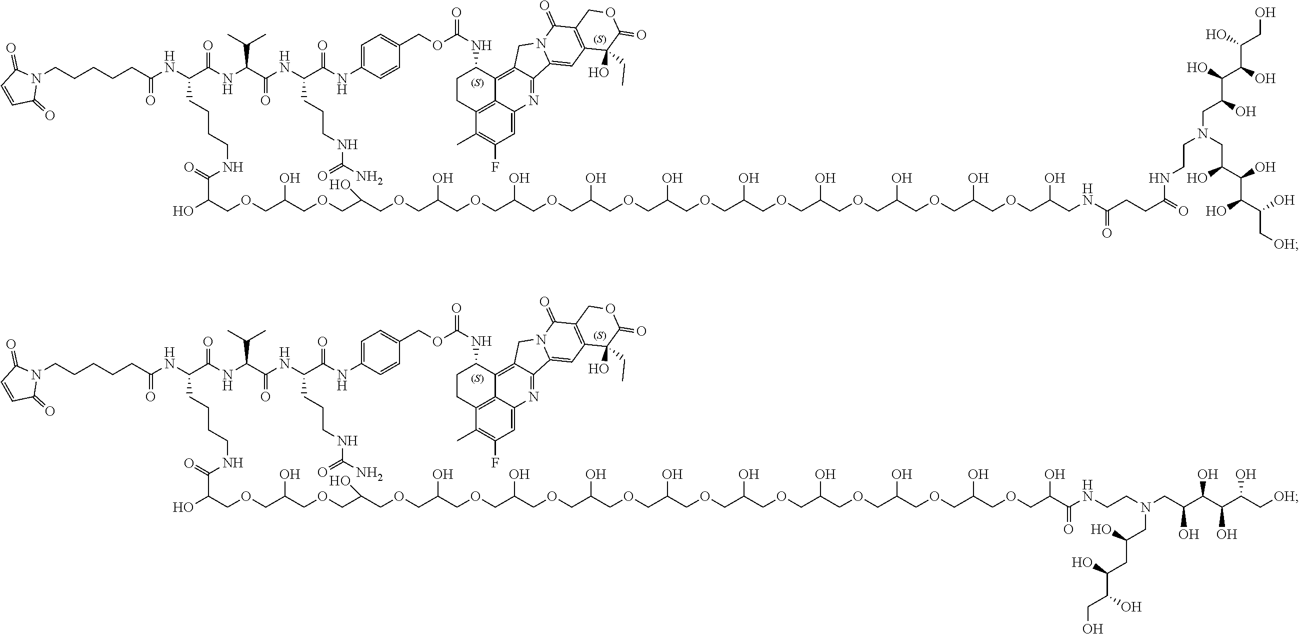 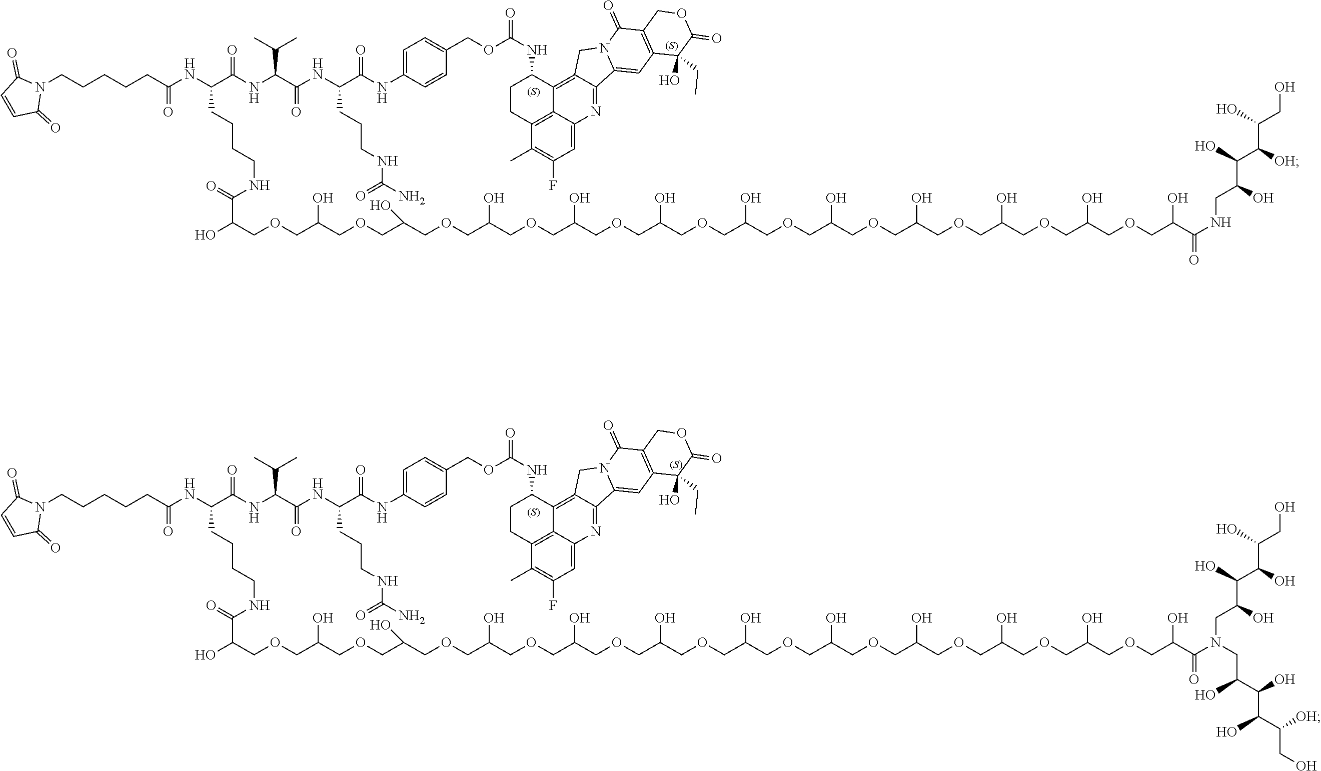 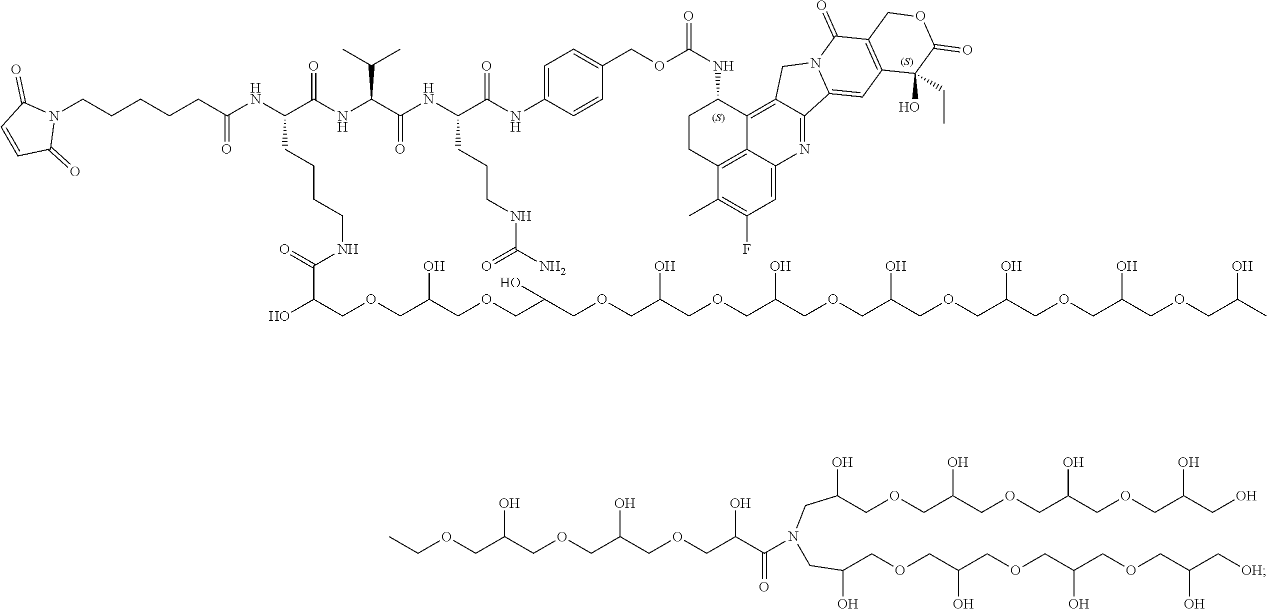 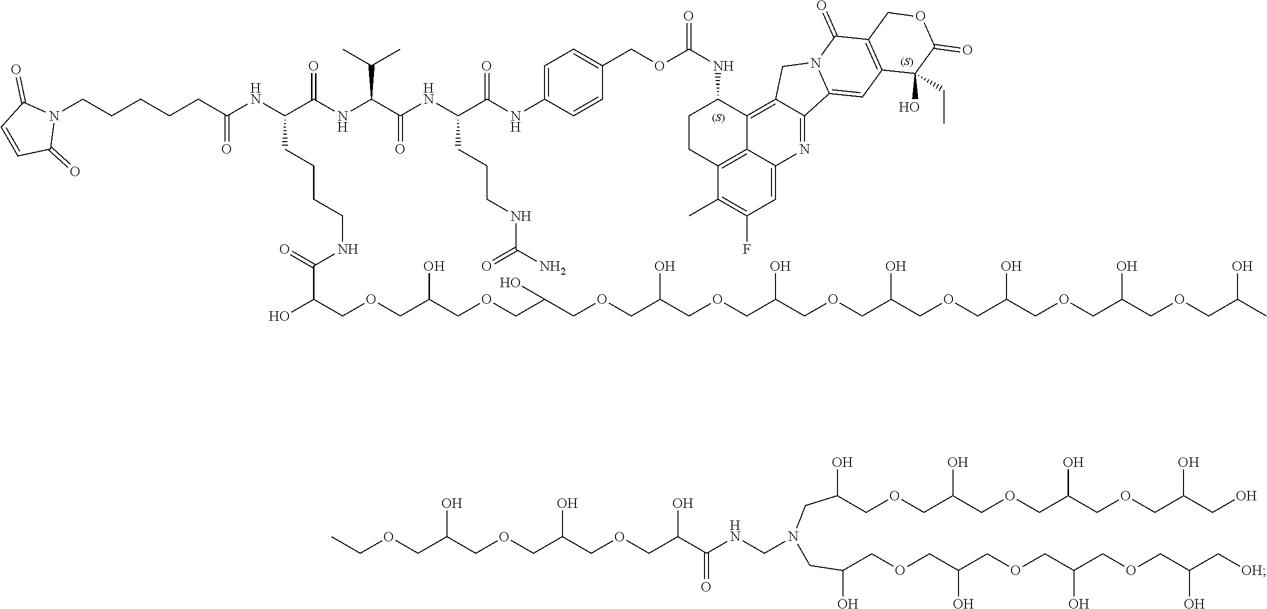 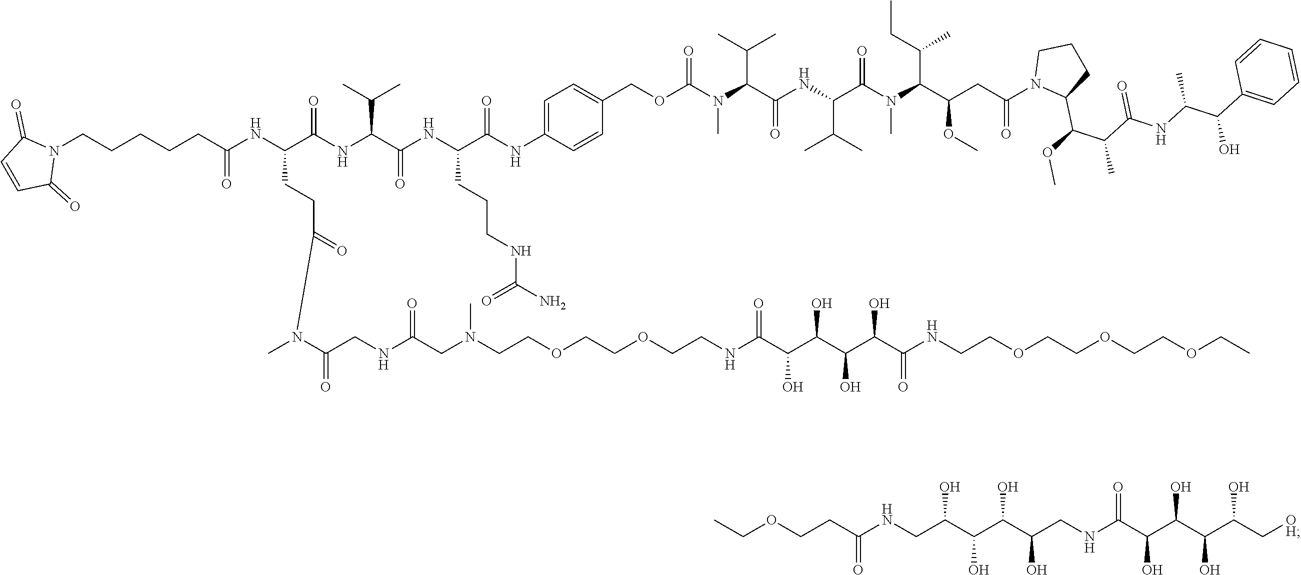 [SMILES: CCOCCOCCOCCNC(=O)[C@H](O)[C@@H](O)[C@H](O)[C@H](O)C(=O)NCCOCCOCCN(C)CC(=O)NCC(=O)N(C)C(=O)CC[C@H](NC(=O)CCCCCN1C(=O)C=CC1=O)C(=O)N[C@H](C(=O)N[C@@H](CCCNC(N)=O)C(=O)Nc1ccc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)[C@@H](O)c2ccccc2)OC)C(C)C)C(C)C)cc1)C(C)C ; CCOCCC(=O)NC[C@H](O)[C@@H](O)[C@H](O)[C@H](O)CNC(=O)[C@H](O)[C@@H](O)[C@H](O)[C@H](O)CO] 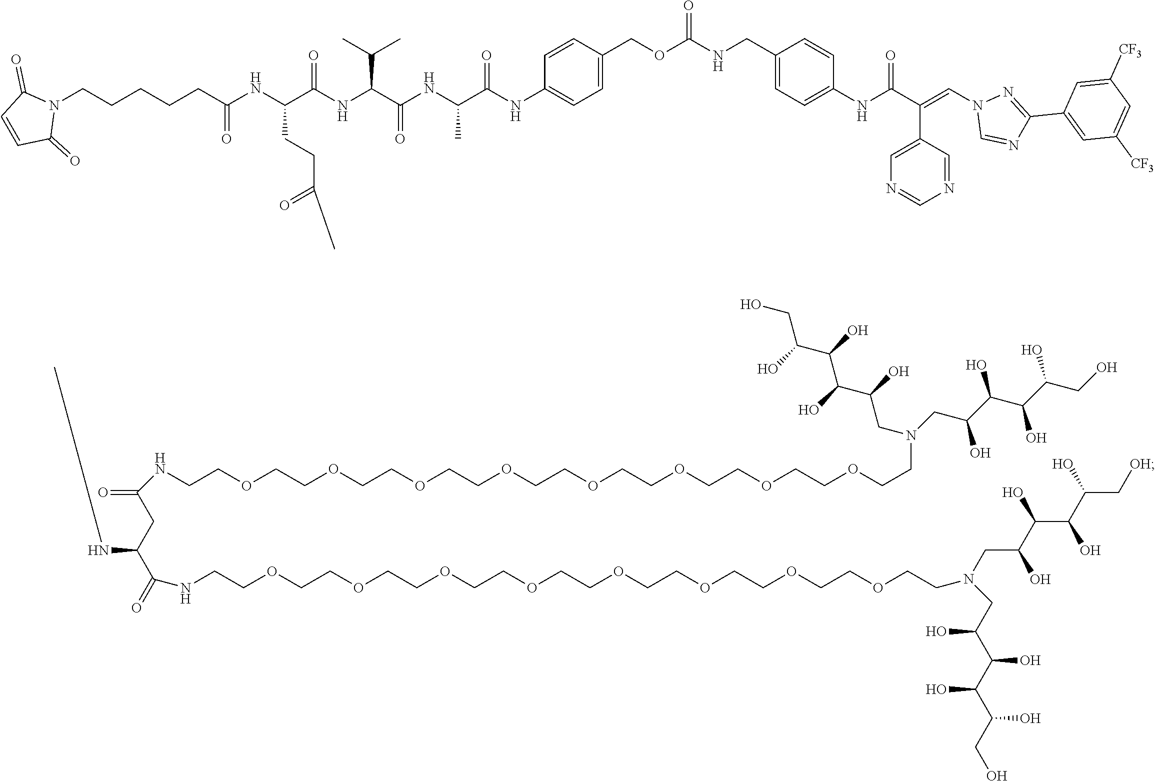 [SMILES: CC(=O)CC[C@H](NC(=O)CCCCCN1C(=O)C=CC1=O)C(=O)N[C@H](C(=O)N[C@@H](C)C(=O)Nc1ccc(COC(=O)NCc2ccc(NC(=O)/C(=C/n3cnc(-c4cc(C(F)(F)F)cc(C(F)(F)F)c4)n3)c3cncnc3)cc2)cc1)C(C)C ; CN[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO] 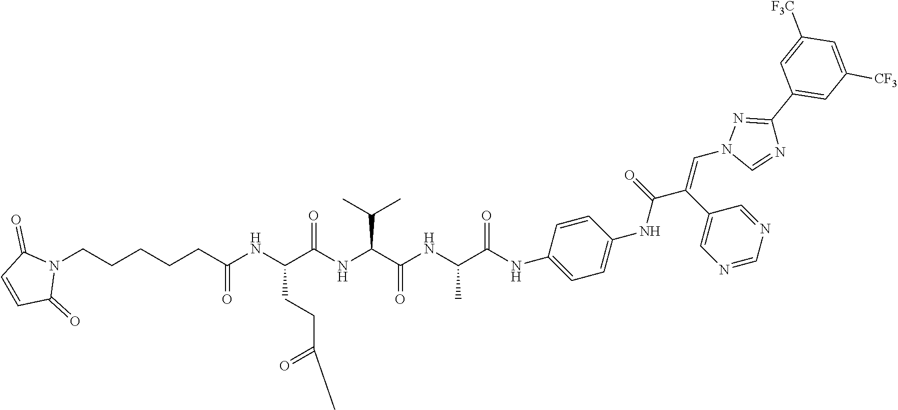 [SMILES: CC(=O)CC[C@H](NC(=O)CCCCCN1C(=O)C=CC1=O)C(=O)N[C@H](C(=O)N[C@@H](C)C(=O)Nc1ccc(NC(=O)/C(=C/n2cnc(-c3cc(C(F)(F)F)cc(C(F)(F)F)c3)n2)c2cncnc2)cc1)C(C)C] 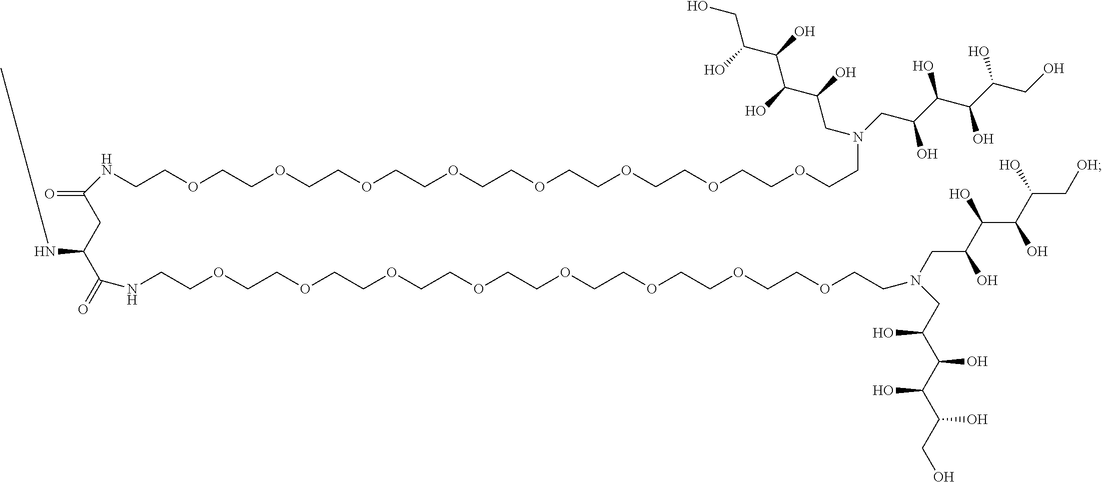 [SMILES: CN[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO] 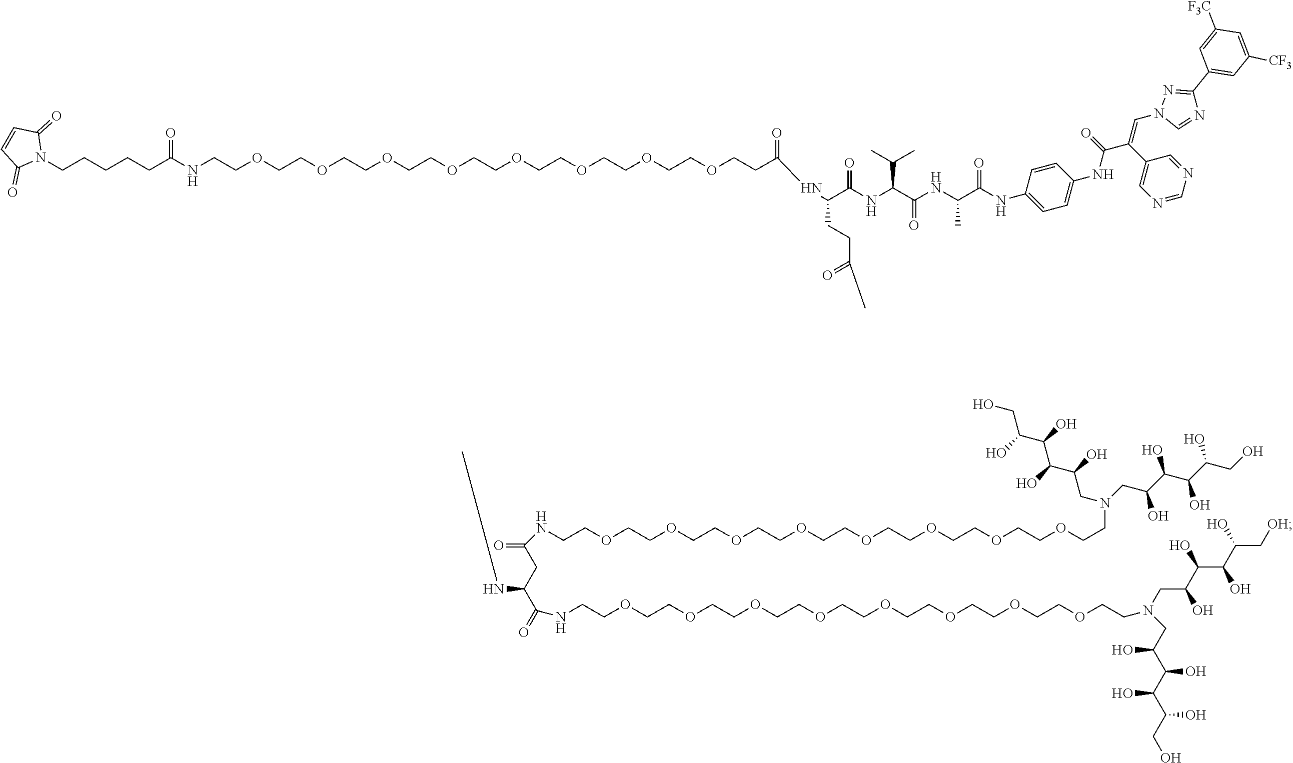 [SMILES: CC(=O)CC[C@H](NC(=O)CCOCCOCCOCCOCCOCCOCCOCCOCCNC(=O)CCCCCN1C(=O)C=CC1=O)C(=O)N[C@H](C(=O)N[C@@H](C)C(=O)Nc1ccc(NC(=O)/C(=C/n2cnc(-c3cc(C(F)(F)F)cc(C(F)(F)F)c3)n2)c2cncnc2)cc1)C(C)C ; CN[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO] 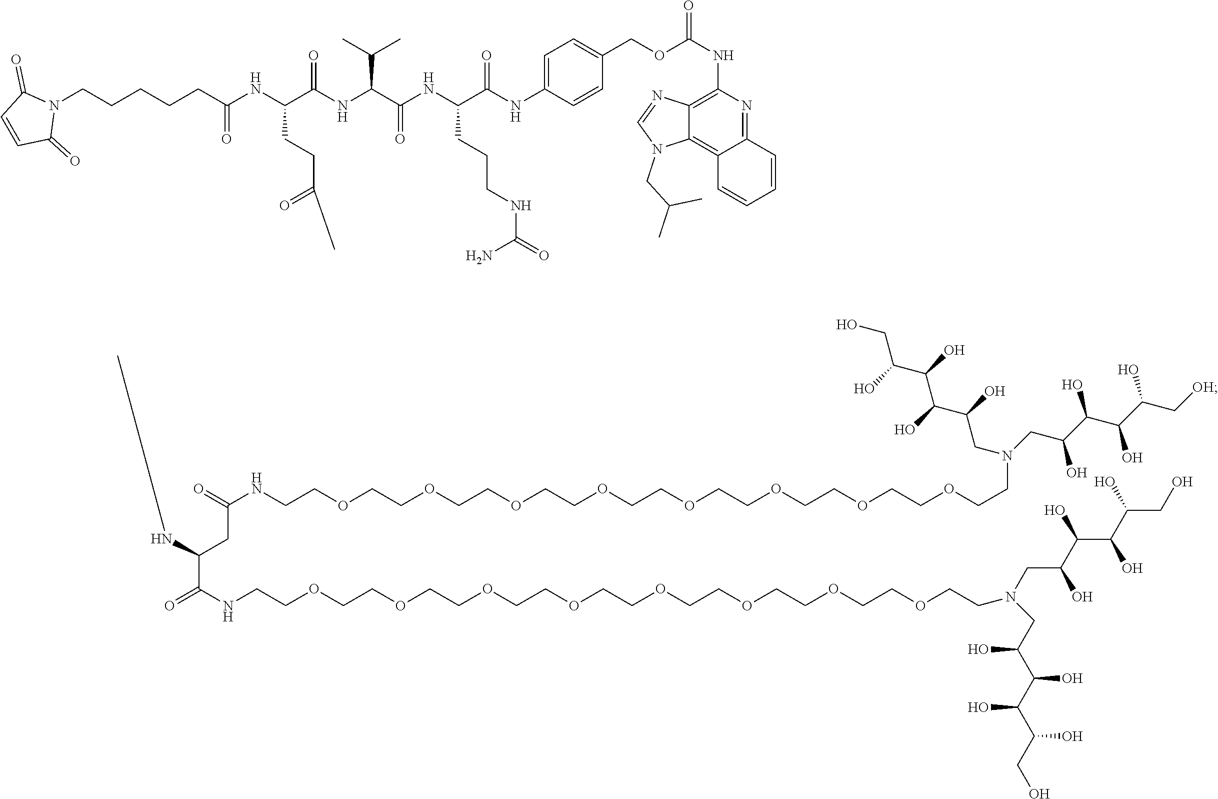 [SMILES: CC(=O)CC[C@H](NC(=O)CCCCCN1C(=O)C=CC1=O)C(=O)N[C@H](C(=O)N[C@@H](CCCNC(N)=O)C(=O)Nc1ccc(COC(=O)Nc2nc3ccccc3c3c2ncn3CC(C)C)cc1)C(C)C ; CN[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO] 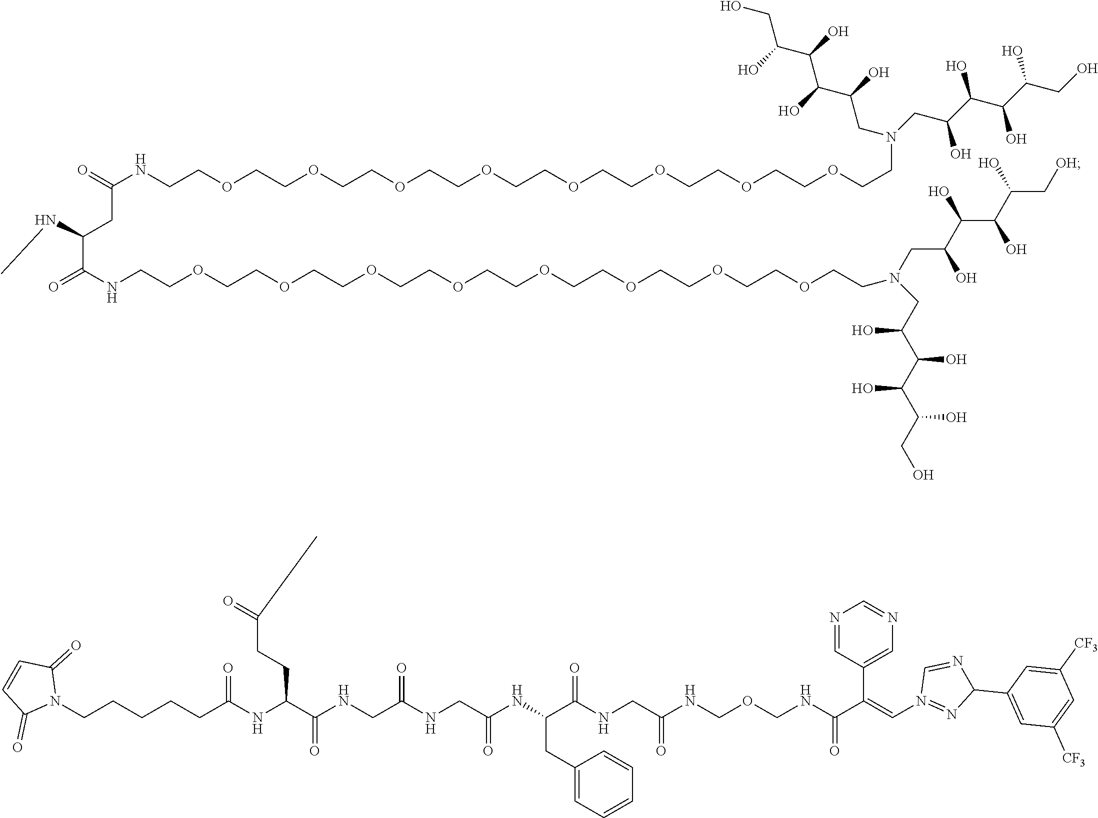 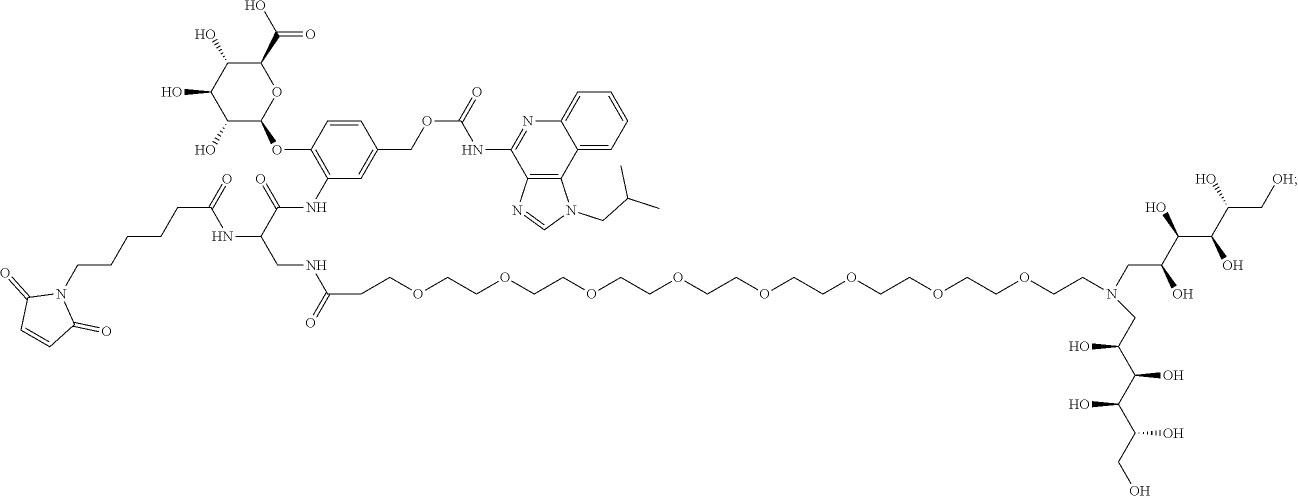 [SMILES: CC(C)Cn1cnc2c(NC(=O)OCc3ccc(O[C@@H]4O[C@H](C(=O)O)[C@@H](O)[C@H](O)[C@H]4O)c(NC(=O)C(CNC(=O)CCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN4C(=O)C=CC4=O)c3)nc3ccccc3c21] 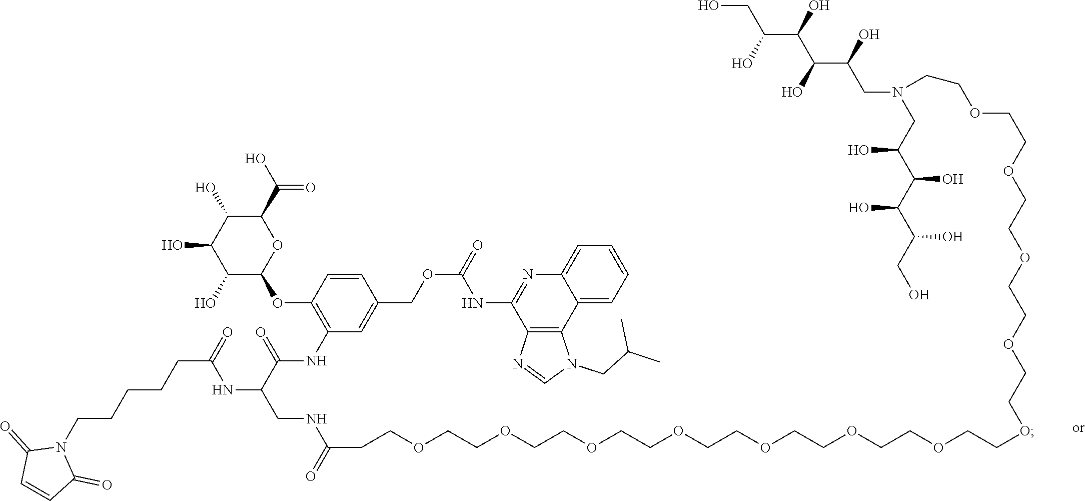 [SMILES: CC(C)Cn1cnc2c(NC(=O)OCc3ccc(O[C@@H]4O[C@H](C(=O)O)[C@@H](O)[C@H](O)[C@H]4O)c(NC(=O)C(CNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN4C(=O)C=CC4=O)c3)nc3ccccc3c21] 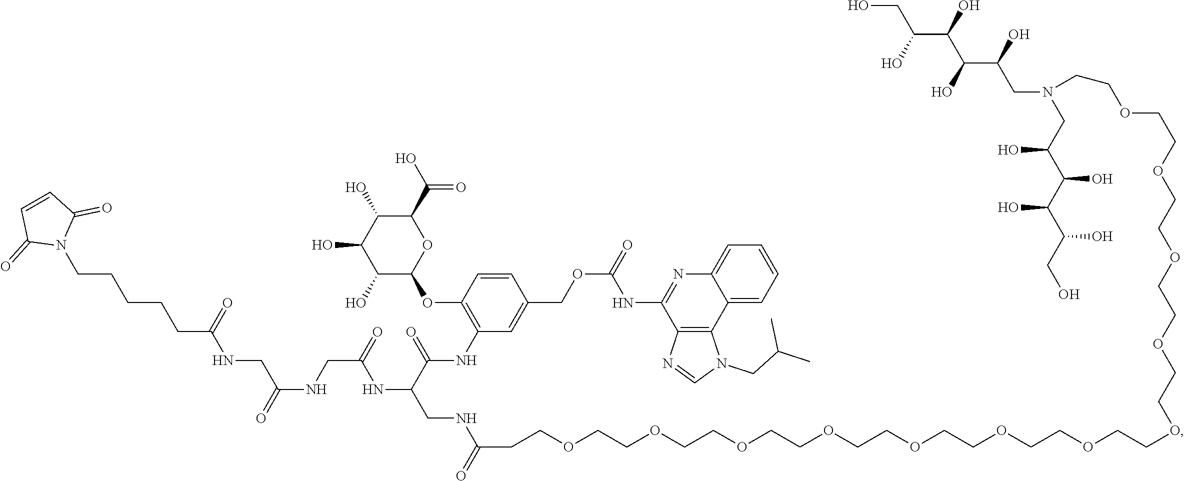 [SMILES: CC(C)Cn1cnc2c(NC(=O)OCc3ccc(O[C@@H]4O[C@H](C(=O)O)[C@@H](O)[C@H](O)[C@H]4O)c(NC(=O)C(CNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CNC(=O)CNC(=O)CCCCCN4C(=O)C=CC4=O)c3)nc3ccccc3c21]
or a stereoisomer thereof.

124. A conjugate comprising a Targeting unit attached to the Drug-linker of any one of claims 100 to 123, wherein the Targeting unit specifically binds to a target molecule.

125. The conjugate of claim 124, wherein the Targeting unit is selected from an antibody or an antigen-binding portion thereof.

126. The conjugate of claim 125, wherein the Targeting unit is a monoclonal antibody, a Fab, a Fab′, an F(ab′), an Fv, a disulfide linked Fc, a scFv, a single domain antibody, a diabody, a bi-specific antibody, or a multi-specific antibody.

127. The conjugate of any one of claim 124 or 125, wherein the Targeting unit is selected from: a scFv1-ScFv2, a ScFv12-Fc-scFv22, a IgG-scFv, a DVD-Ig, a triomab/quadroma, a two-in-one IgG, a scFv2-Fc, a TandAb, and an scFv-HSA-scFv.

128. The conjugate of claim 124, wherein the Targeting unit is a diabody, a DART, an anticalin, an affibody, an avimer, a DARPin, or an adnectin.

129. The conjugate of any one of claims 124-128, wherein the Targeting unit is mono-specific.

130. The conjugate of any one of claims 124-129, wherein the Targeting unit is bivalent.

131. The conjugate of any one of claims 124-129, wherein the Targeting unit is bispecific.

132. The conjugate of any one of claims 124-131, wherein the average drug loading (p^{load}) of the conjugate is from about 1 to about 8, about 2, about 4, about 6, about 8, about 10, about 12, about 14, about 16, about 3 to about 5, about 6 to about 8, or about 8 to about 16.

133. The conjugate of any one of claims 124-132, selected from the following: 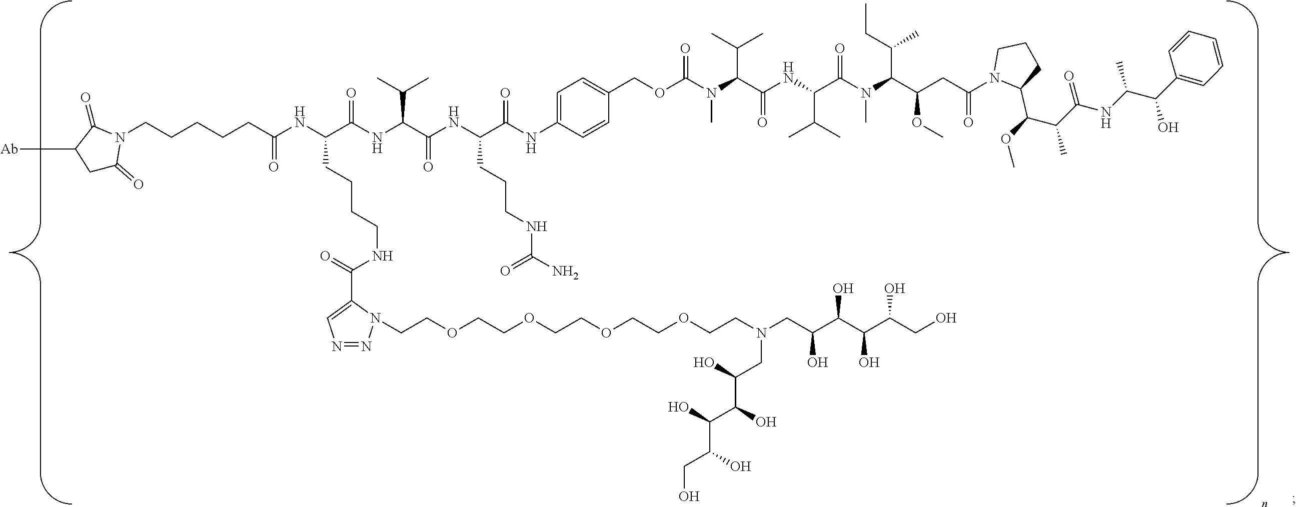 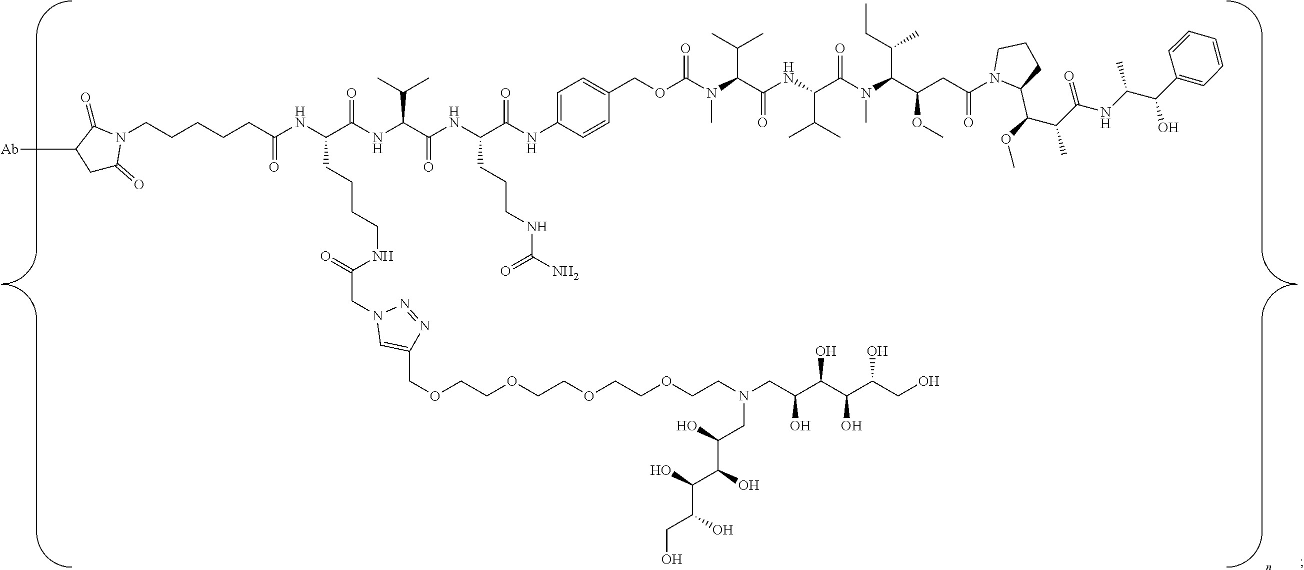 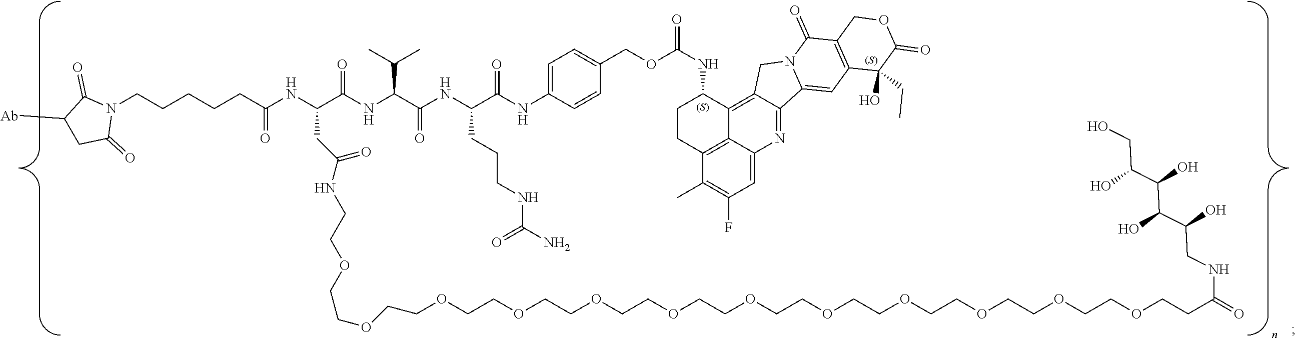 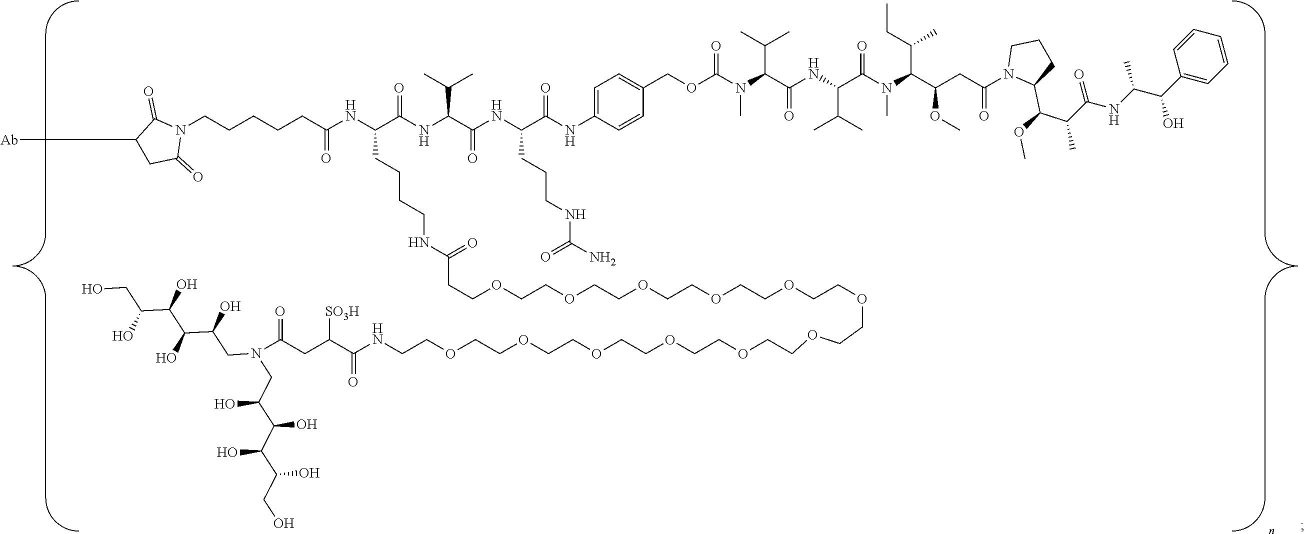 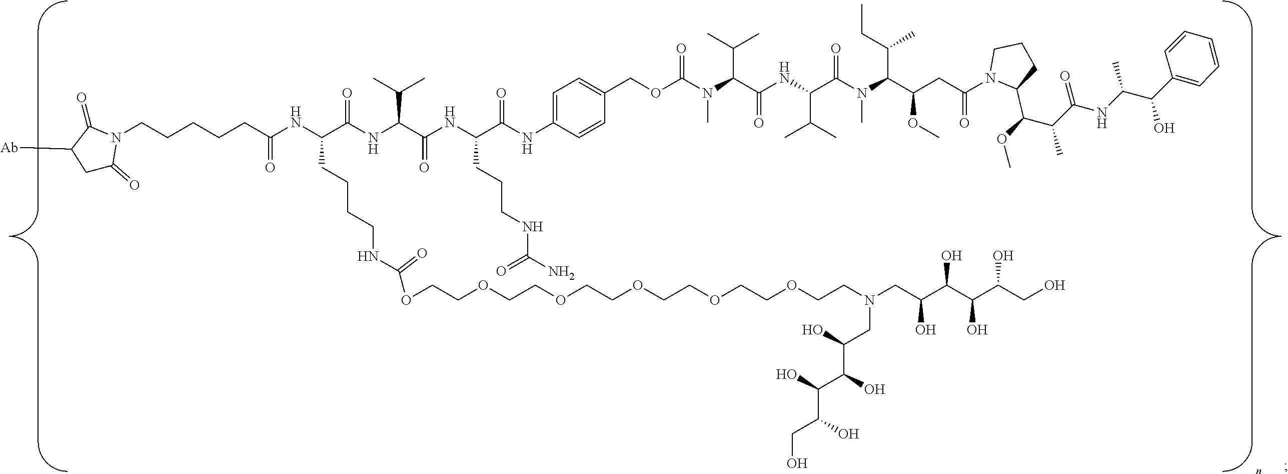 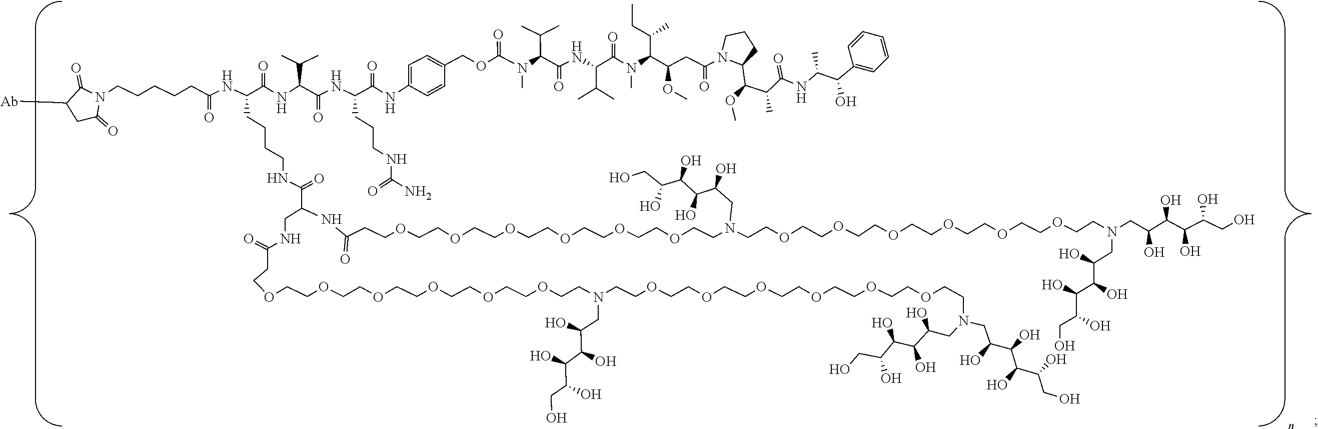 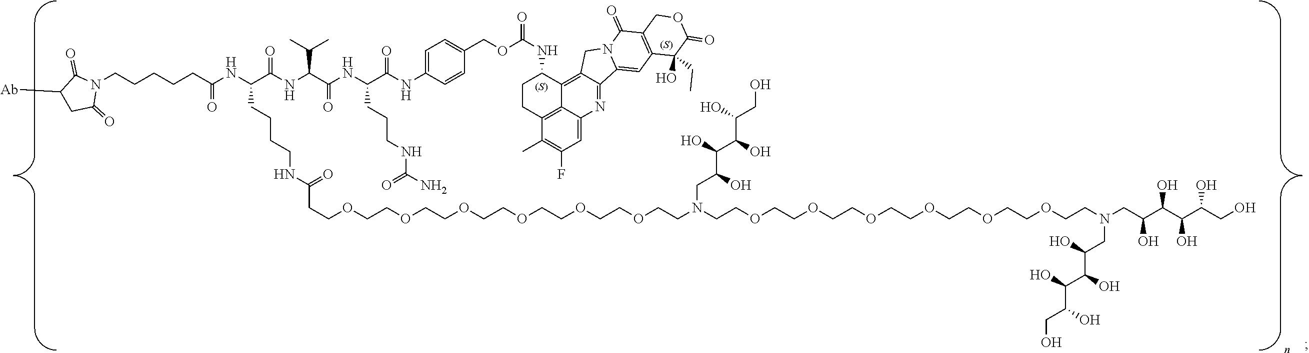 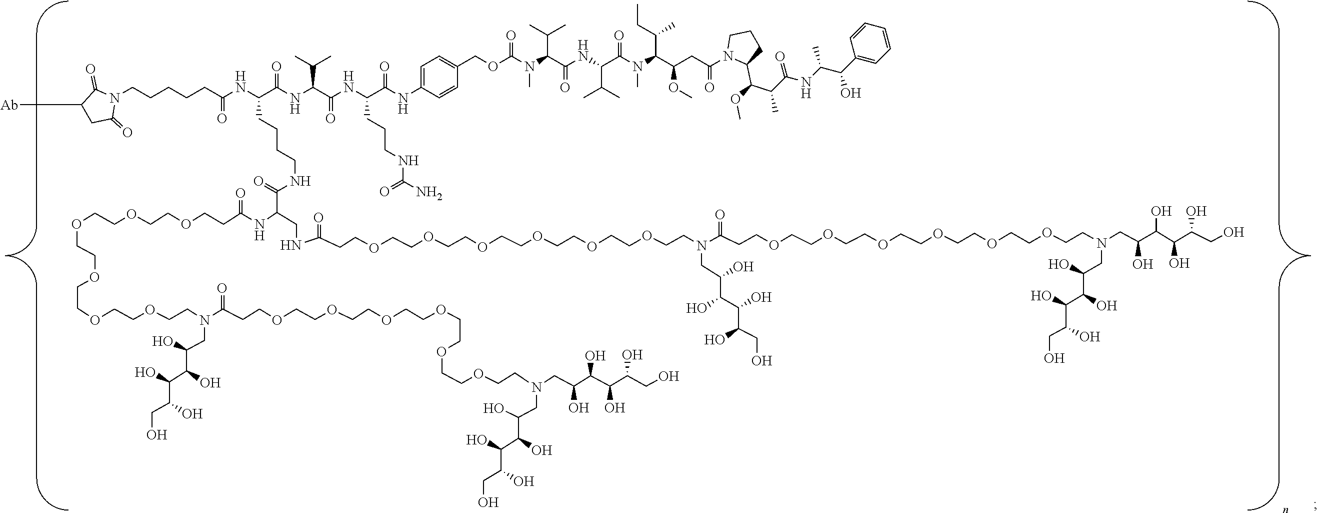 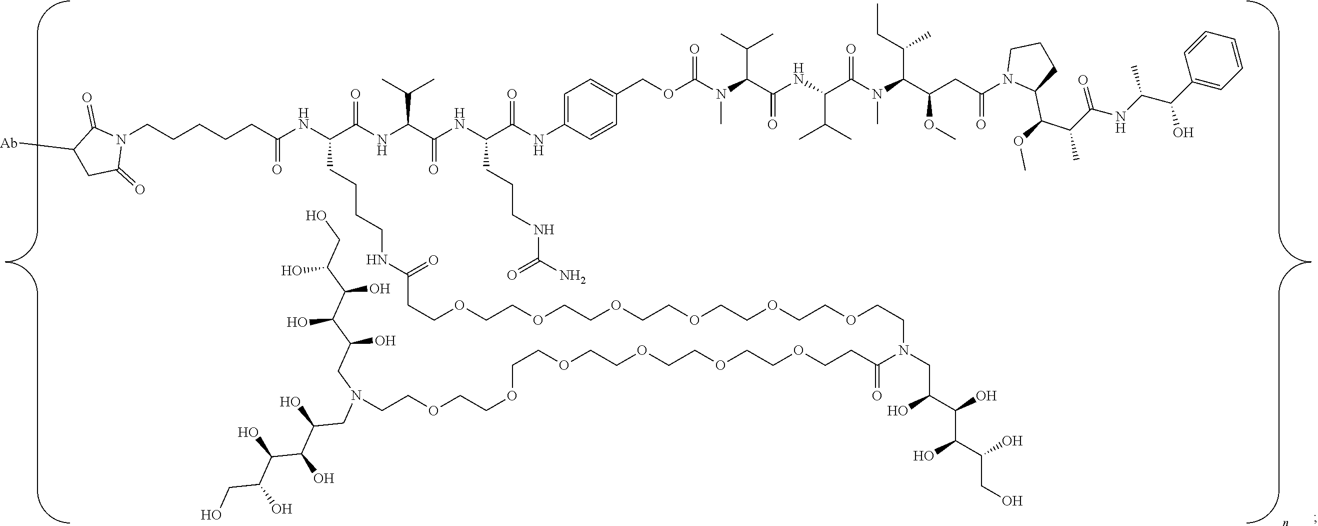 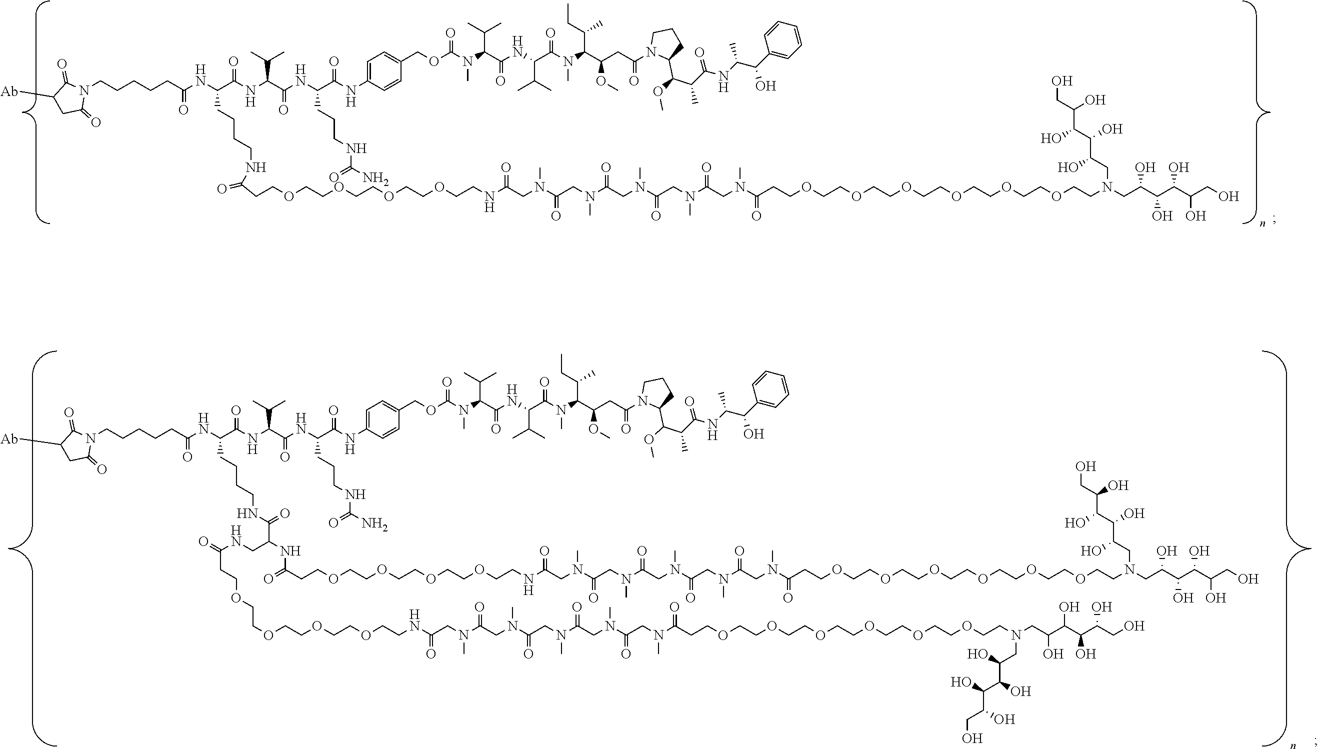 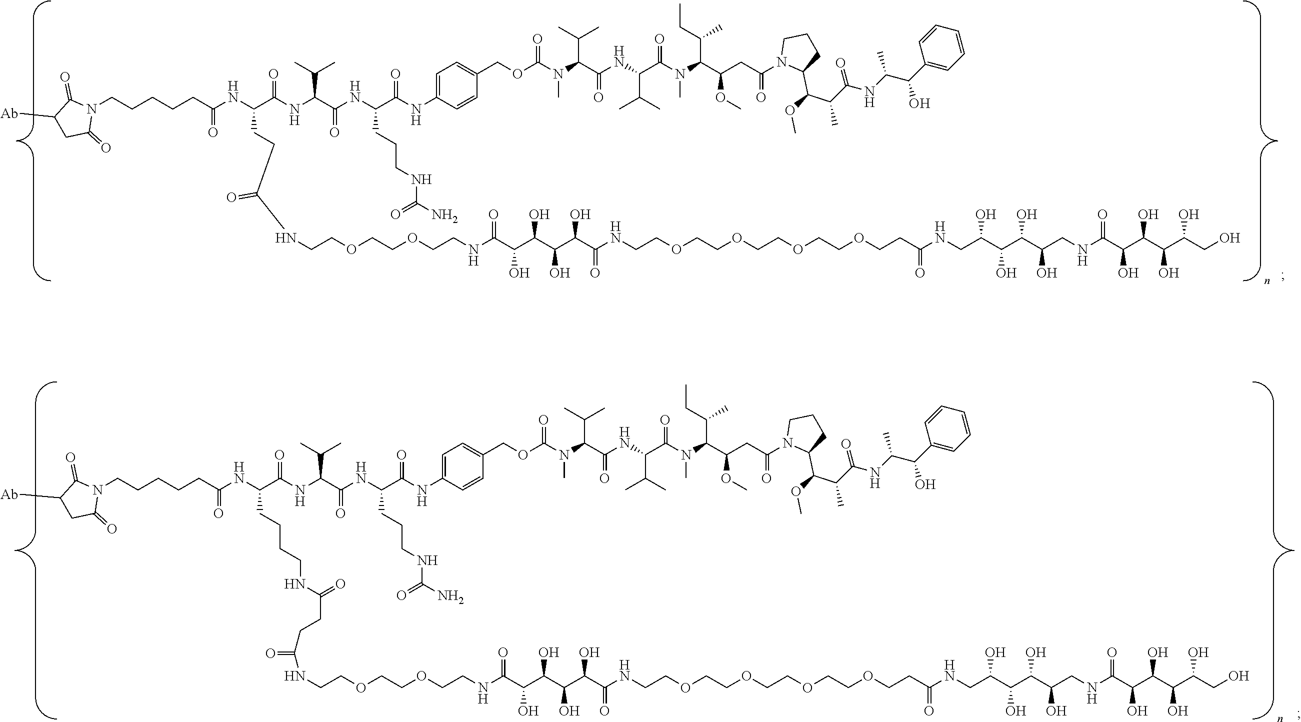 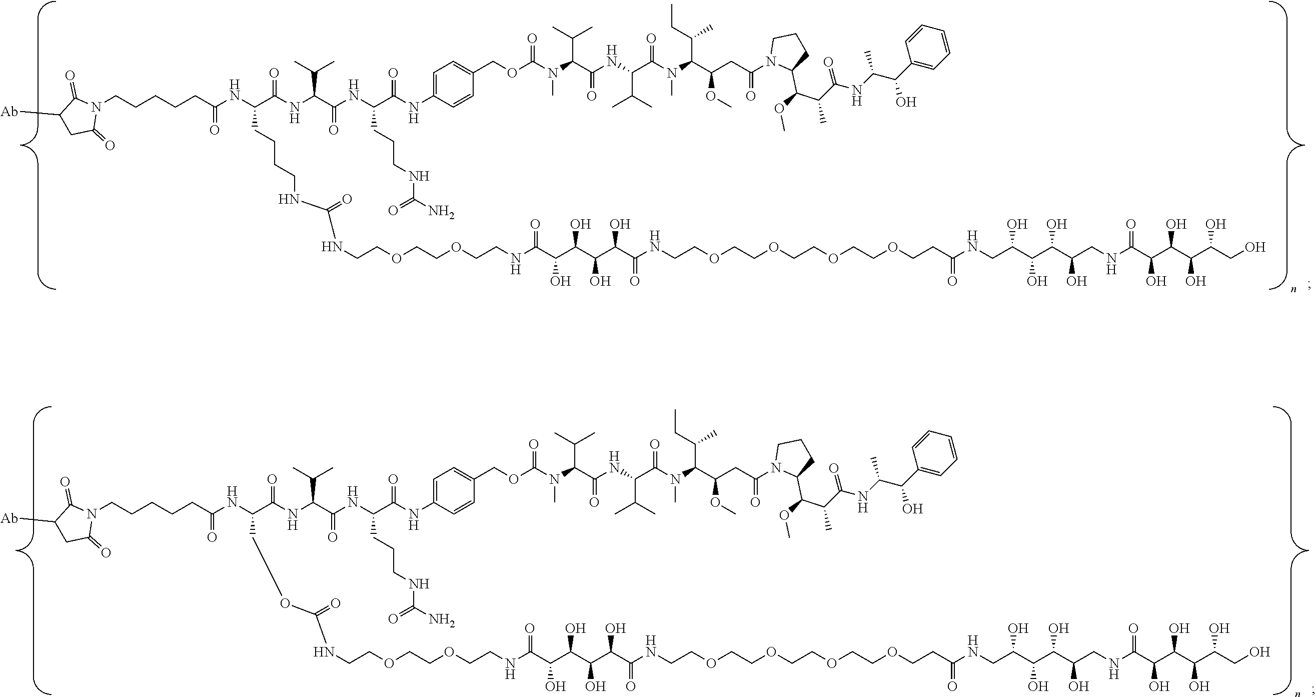 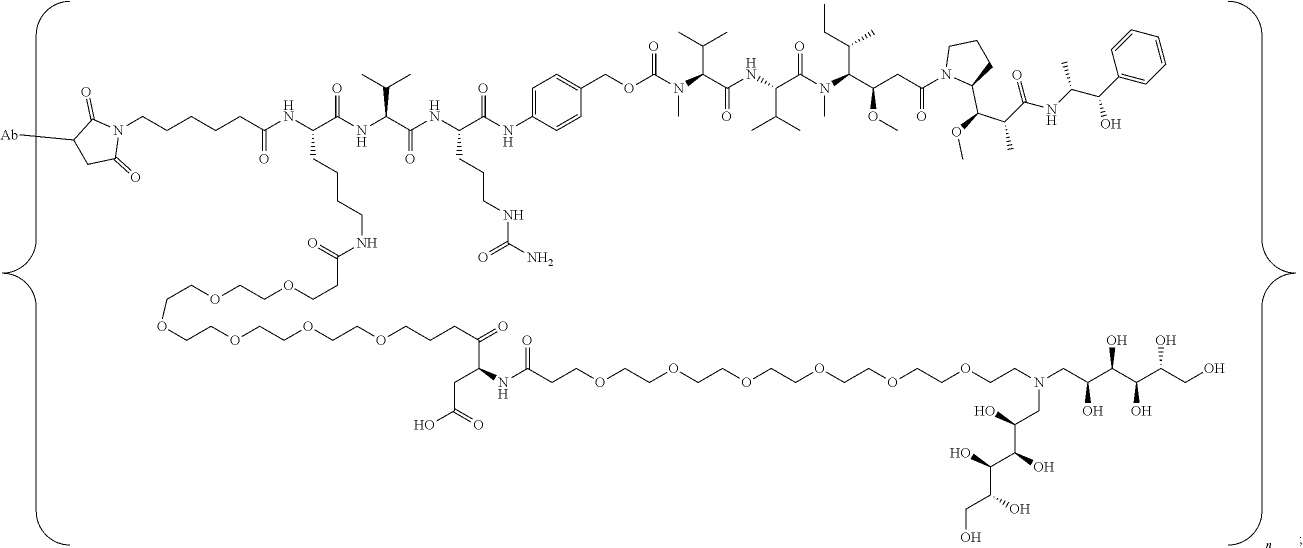 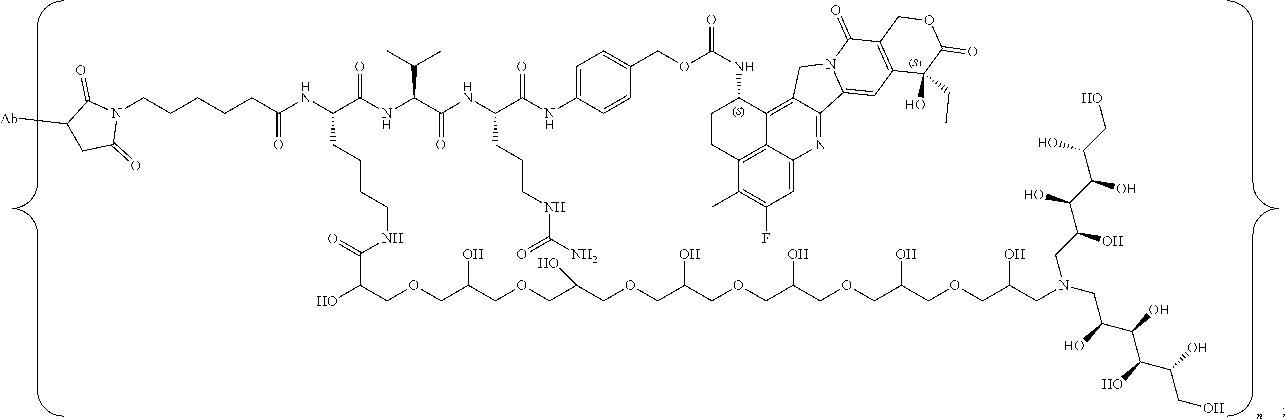 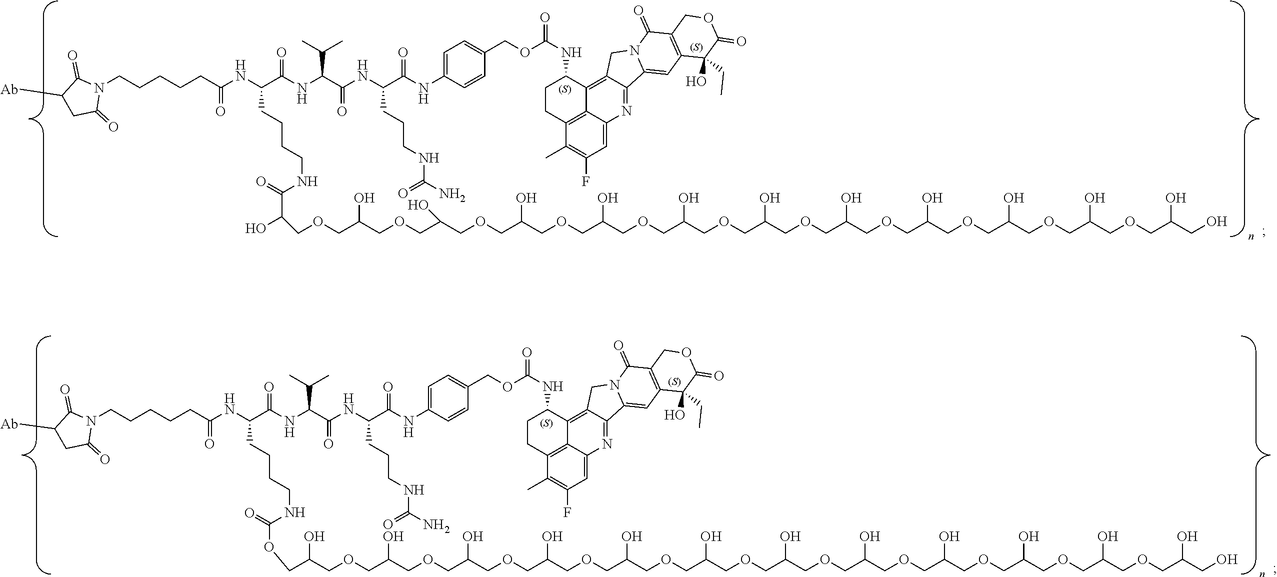 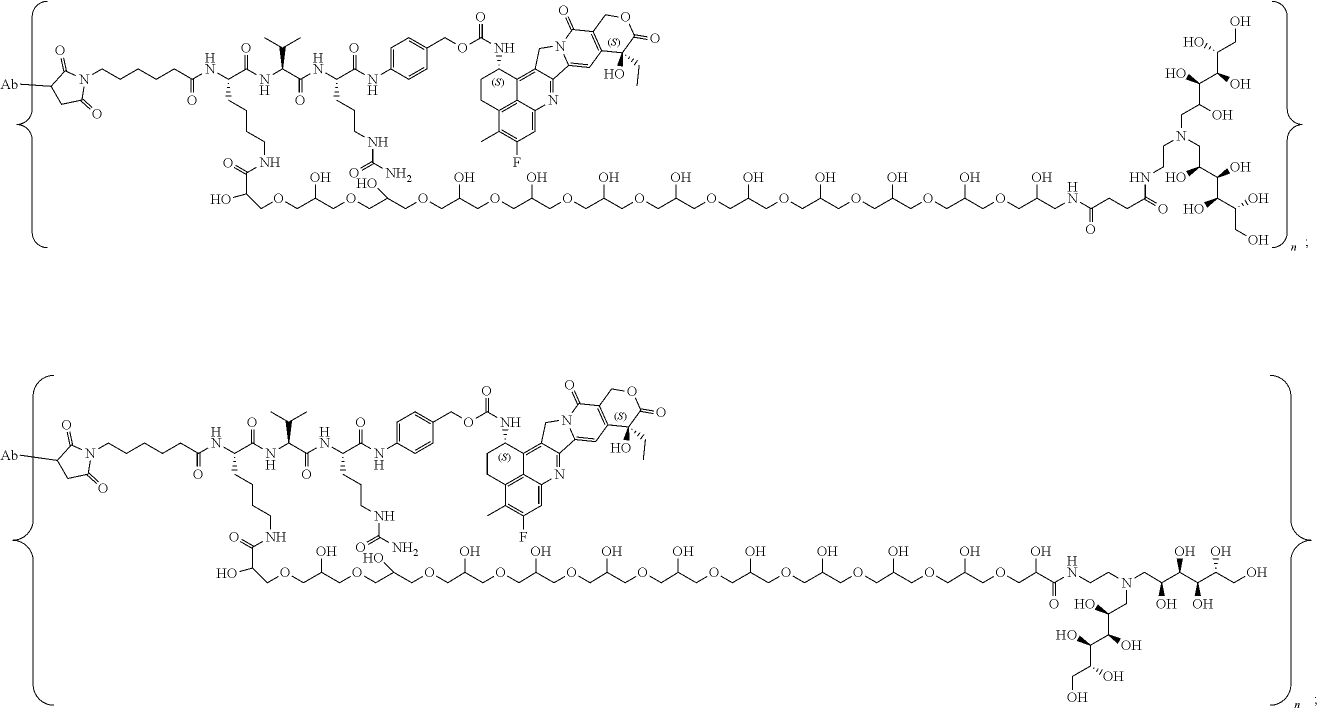 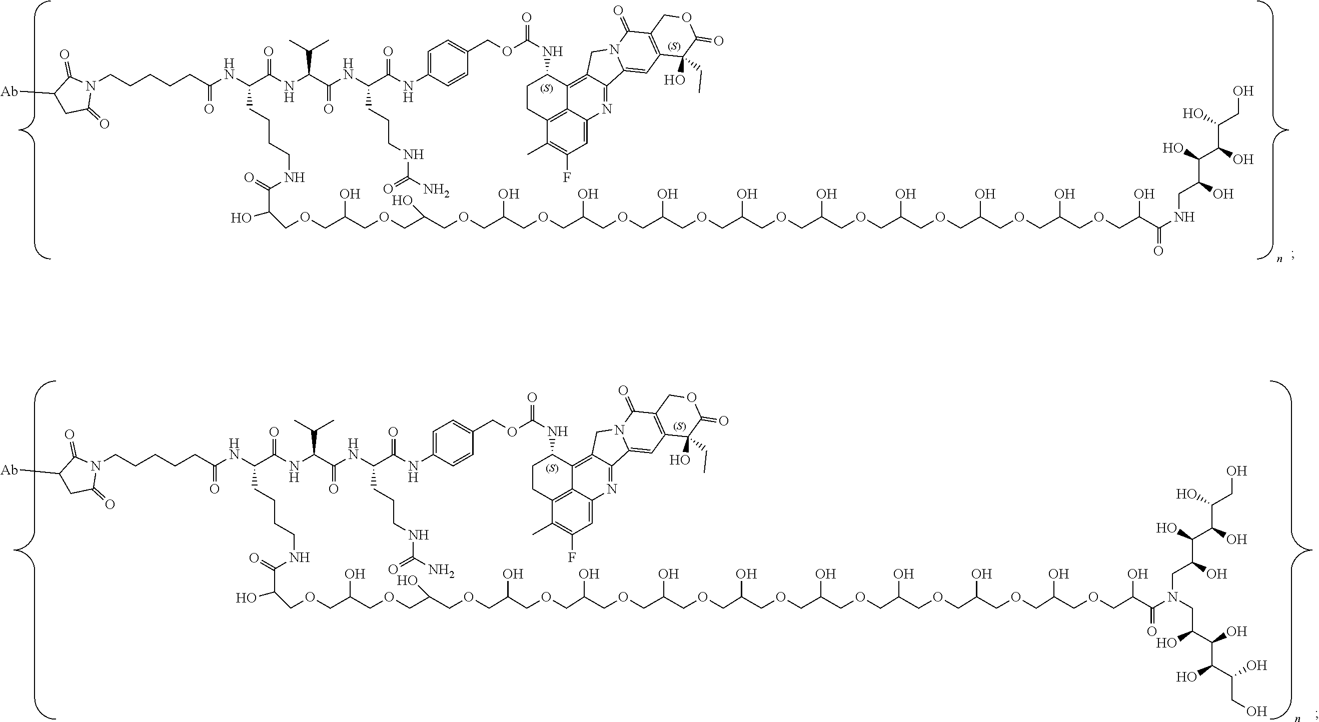 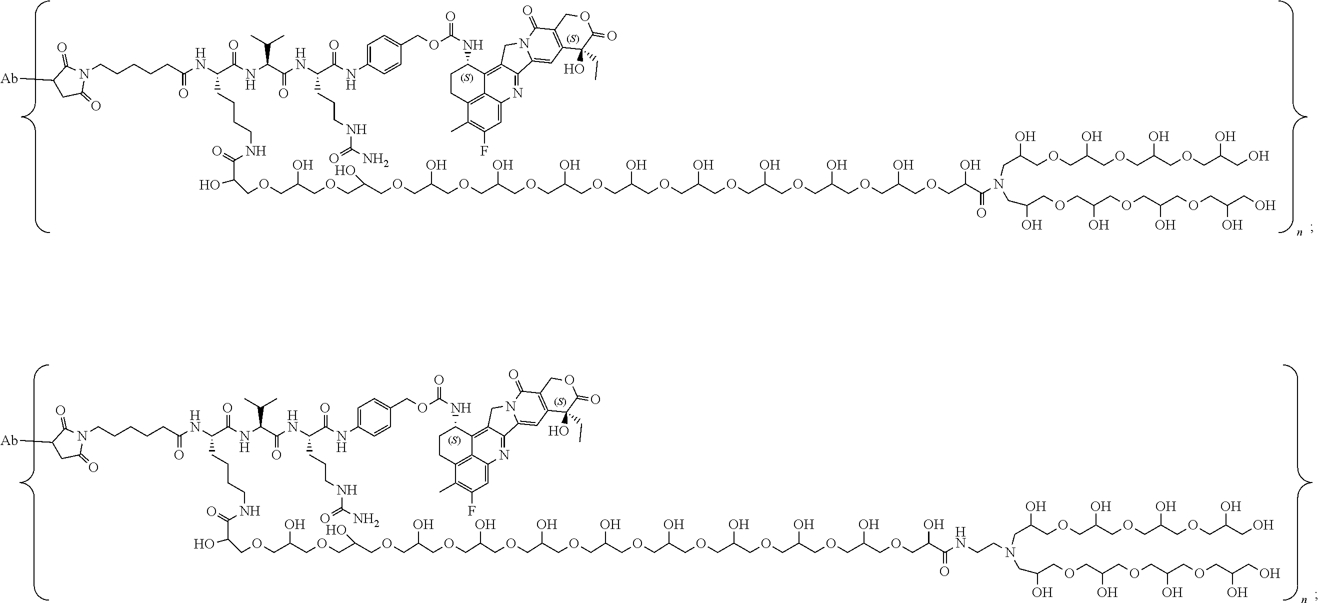 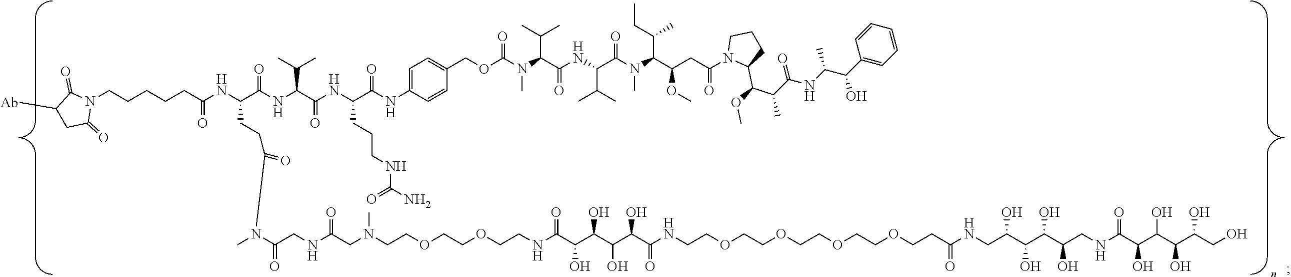 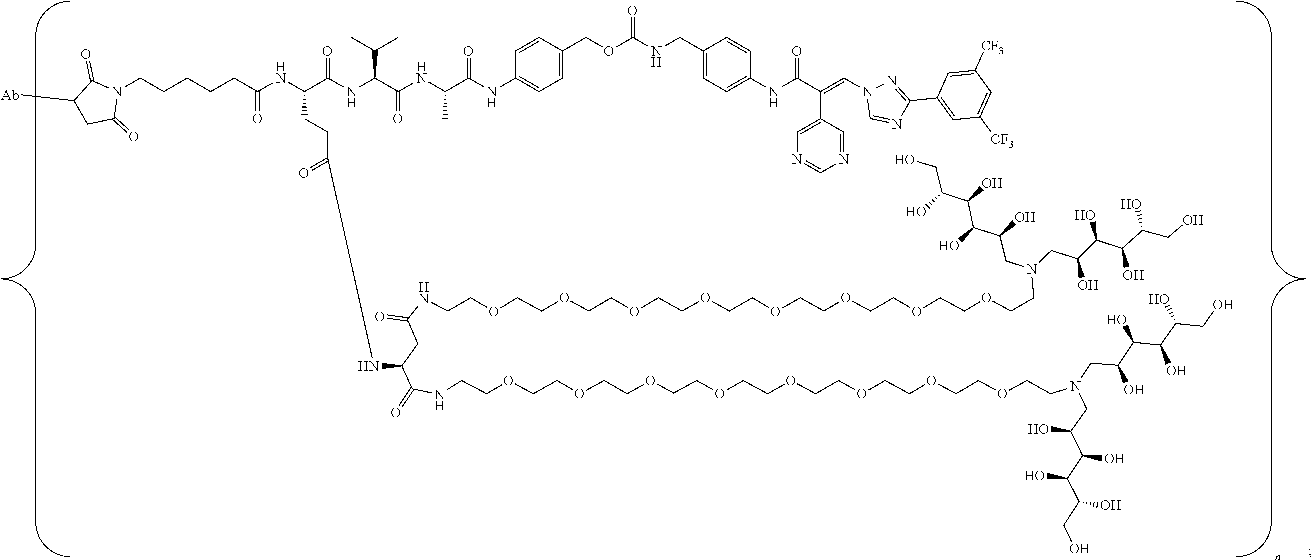 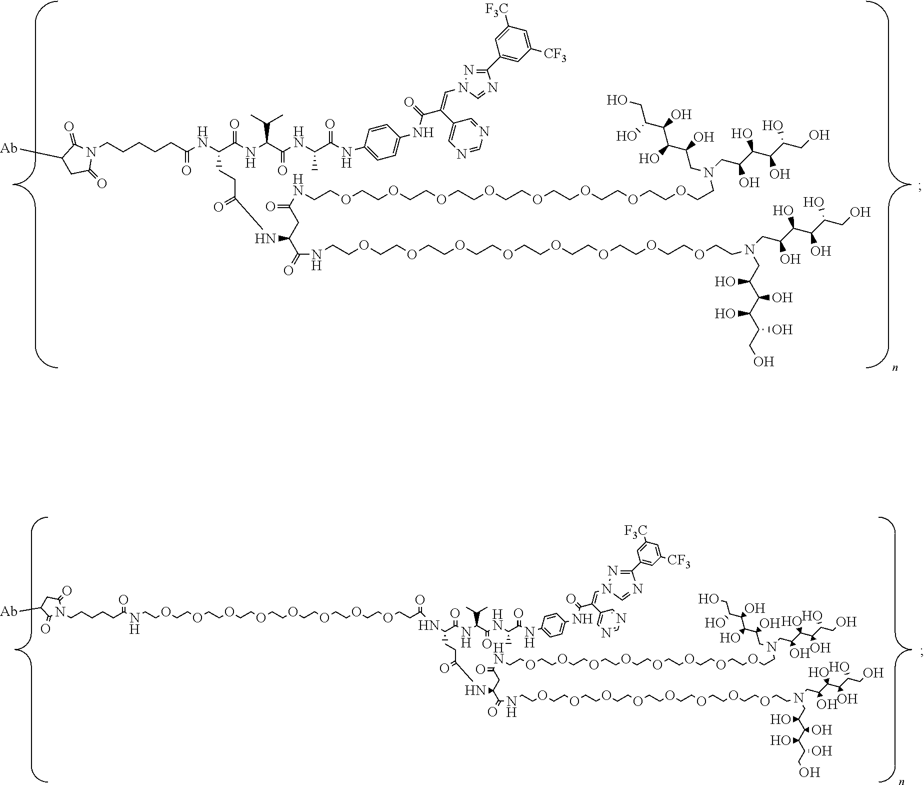 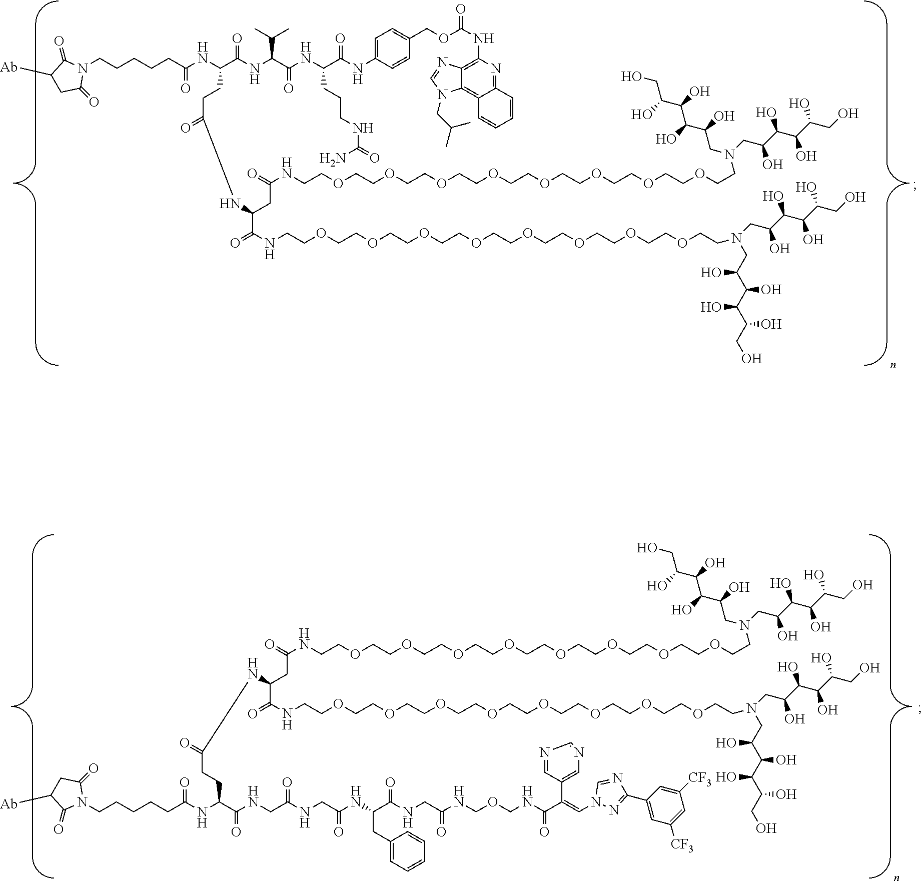 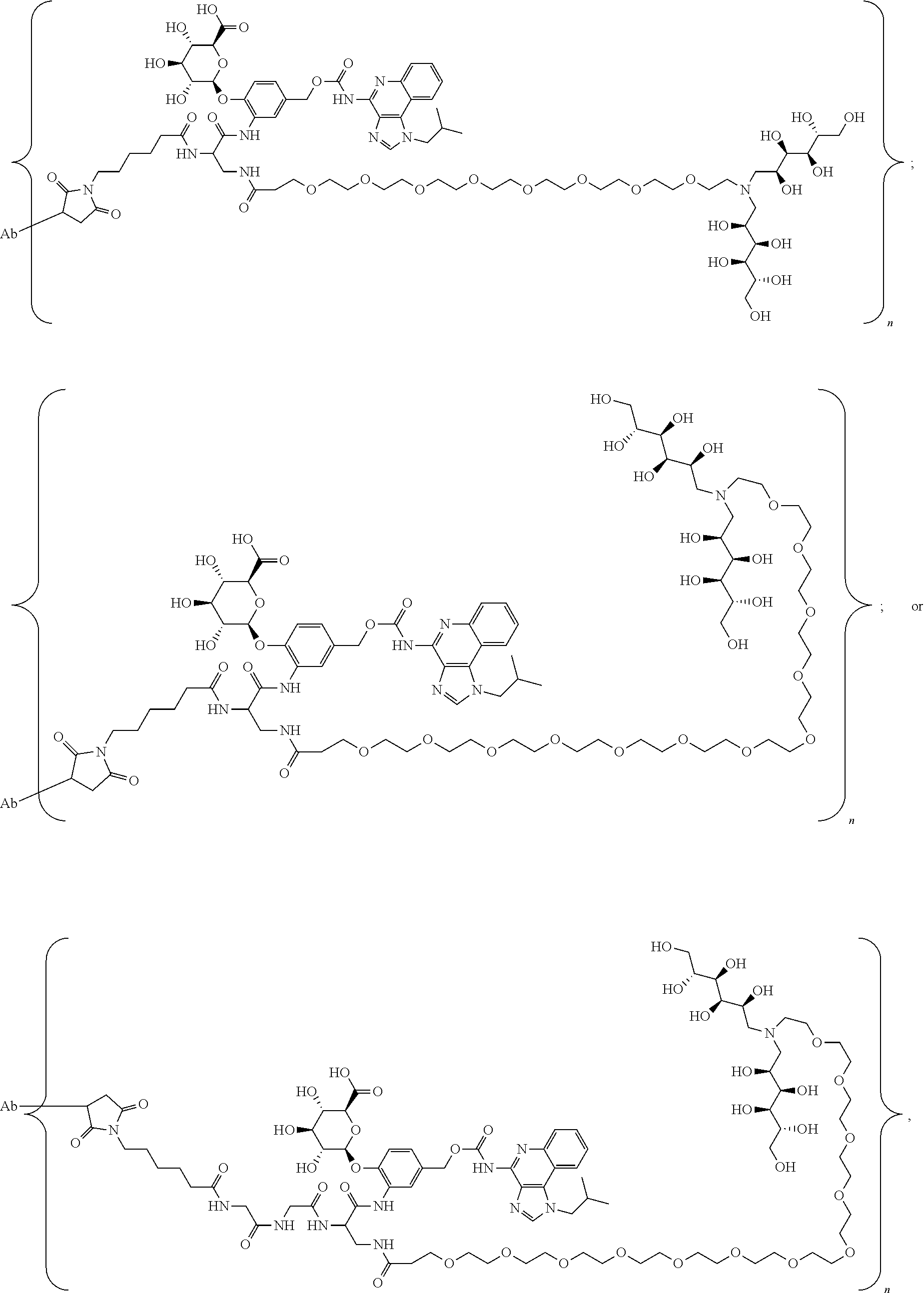
or a stereoisomer thereof.

134. The conjugate of claim 133, wherein the target molecule is CD19, CD20, CD30, CD33, CD70, LIV-1, HER2, or EGFRv3.

135. The conjugate of any one of claims 124-134, wherein the target molecule is a cancer associated antigen.

136. The conjugate of any one of claims 124-133, wherein the target molecule is CD19, CD20, CD30, CD33, CD38, CA125, MUC-1, prostate-specific membrane antigen (PSMA), CD44 surface adhesion molecule, mesothelin (MLSN), carcinoembryonic antigen (CEA), epidermal growth factor receptor (EGFR), EGFRvIII, vascular endothelial growth factor receptor-2 (VEGFR2), HER2, high molecular weight-melanoma associated antigen (HMW-MAA), MAGE-A1, IL-13R-a2, GD2, 1p19q, ABL1, AKT1, ALK, APC, AR, ATM, BRAF, BRCA1, BRCA2, cKIT, cMET, CSF1R, CTNNB1, FGFR1, FGFR2, FLT3, GNA11, GNAQ, GNAS, HRAS, IDH1, IDH2, JAK2, KDR (VEGFR2), KRAS, MGMT, MGMT-Me, MLH1, MPL, NOTCH1, NRAS, PDGFRA, Pgp, PIK3CA, PR, PTEN, RET, RRM1, SMO, SPARC, TLE3, TOP2A, TOPO1, TP53, TS, TUBB3, VHL, CDH1, ERBB4, FBXW7, HNF1A, JAK3, NPM1, PTPN11, RB1, SMAD4, SMARCB1, STK1, MLH1, MSH2, MSH6, PMS2, ROS1, ERCC1, 5T4 (TPBG), B7-H3, CCR7, CD105, CD22, CD46, CD47, CD56, CD70, CD71, CD79b, CDH6, CLDN6, CLDN18.2, CLEC12A, DLL3, DR5, ERBB3 (HER3), EPCAM, FOLR1, IGF1R, IL2RA (CD25), IL3RA, ITGB6, LIV-1, LRRC15, mesothelin (MSLN), NaPi2b (SLC34A2), nectin-4, PTK7, ROR1, SEZ6, SLC44A4, SLITRK6, Tissue Factor (TF), TROP2 or B7-H4.

137. The conjugate of any one of claims 124-126-130, 131, or 132, wherein the Targeting unit is an antibody, or fragment thereof, comprising rituximab (Rituxan®), trastuzumab (Herceptin®), pertuzumab (Perjeta®)), bevacizumab (Avastin®), ranibizumab (Lucentis®), cetuximab (Erbitux®), alemtuzumab (Campath®), panitumumab (Vectibix®), ibritumomab tiuxetan (Zevalin®), tositumomab (Bexxar®), ipilimumab, zalutumumab, dalotuzumab, figitumumab, ramucirumab, galiximab, farletuzumab, ocrelizumab, ofatumumab (Arzerra®), tositumumab, ibritumomab, the CD20 antibodies 2F2 (HuMax-CD20), 7D8, IgM2C6, IgG1 2C6, 11B8, B1, 2H7, LT20, IFS or AT80, daclizumab (Zenapax®), anti-SLITRK6 antibodies including hu 1H2-03, or anti-LHRH receptor antibodies including clone A9E4, F1G4, AT2G7, GNRH03, or GNRHR2.

138. A pharmaceutical composition comprising the conjugate of any one of claims 124-137 and a pharmaceutically acceptable carrier.

139. A method of treating a subject in need thereof, comprising administering to the subject a conjugate of any one of claims 124-137, or the pharmaceutical composition of claim 138, wherein the subject has cancer or an autoimmune disease and the conjugate binds to a target antigen associated with the cancer or autoimmune disease.

140. A Polar group represented by the formula: 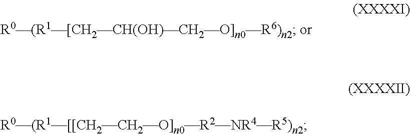
or
a salt of anyone thereof, wherein:
R⁰ has one of the following structures: 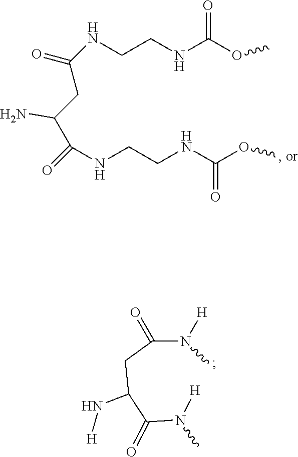
each R¹ is independently a bond, or C₁-C₃ alkylene group;
R² is independently selected from a C₁-C₃ alkylene group;
R⁴ and R⁵ are each independently selected from a H, or polyhydroxyl group, wherein R⁴ and R⁵ are not both H;
each R⁶ is independently C₁-C₆alkylene-C(OH)H—C₁-C₆alkylene-NR⁷R⁸, —C(O)N(R^{N})—C₁-C₆alkylene-NR⁴R⁵;
R^{N} is H or C₁-C₄alkyl;
R⁷ and R⁸ are each independently selected from a H, polyhydroxyl group, or —C(O)— polyhydroxyl group;
each n0 is independently 1 to 26; and
n2 is 1 or 2.

141. The Polar group of claim 140, represented by the formula: 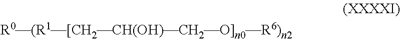
wherein
R⁰ has one of the following structures: 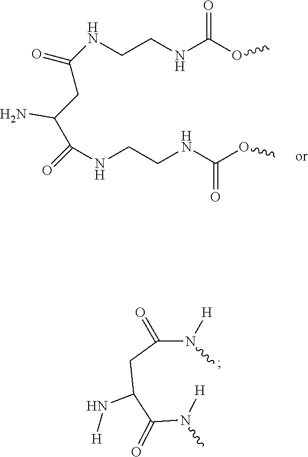
each R¹ is independently a bond, or C₁-C₃ alkylene group;
R⁴ and R⁵ are each independently selected from a H, or polyhydroxyl group, wherein R⁴ and R⁵ are not both H;
each R⁶ is independently C₁-C₆alkylene-C(OH)H—C₁-C₆alkylene-NR⁷R⁸, —C(O)N(R^{N})—C₁-C₆alkylene-NR⁴R⁵;
R^{N} is H or C₁-C₄alkyl;
R⁷ and R⁸ are each independently selected from a H, polyhydroxyl group, or —C(O)— polyhydroxyl group;
each n0 is independently 1 to 26; and
n2 is 1 or 2.

142. The Polar group of claim 140 or 141, wherein R⁰ is

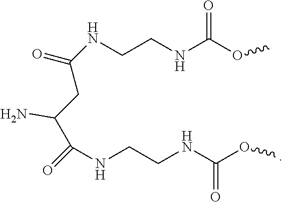

143. The Polar group of claim 140 or 141, wherein R⁰ is

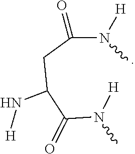

144. The Polar group of any one of claims 140 to 143, wherein R¹ is a bond.

145. The Polar group of any one of claims 140 to 144, wherein n0 is 4 to 10.

146. The Polar group of any one of claims 140 to 145, wherein n0 is 6.

147. The Polar group of any one of claims 140 to 146, wherein R⁶ is C₁-C₆alkylene-C(OH)H—C₁-C₆alkylene-NR⁷R⁸.

148. The Polar group of any one of claims 140 to 147, wherein R⁶ is C₁-alkylene-C(OH)H—C₁-alkylene-NR⁷R⁸.

149. The Polar group of any one of claims 140 to 148, wherein R⁷ is polyhydroxyl group.

150. The Polar group of any one of claims 140 to 149, wherein R⁸ is polyhydroxyl group.

151. The Polar group of any one of claims 140 to 145, wherein R⁶ is —C(O)N(R^{N})—C₁-C₆alkylene-NR⁴R⁵.

152. The Polar group of any one of claims 140 to 145, or 151, wherein R⁴ is polyhydroxyl group.

153. The Polar group of any one of claims 140 to 145, or 151, wherein R⁵ is polyhydroxyl group.

154. The Polar group of any one of claims 140 to 145, or 151 to 153, wherein R^{N} is H.

155. The Polar group of any one of claims 140 to 145, or 151 to 154, wherein n2 is 2.

156. The Polar group of claim 140, wherein the Polar group is selected from

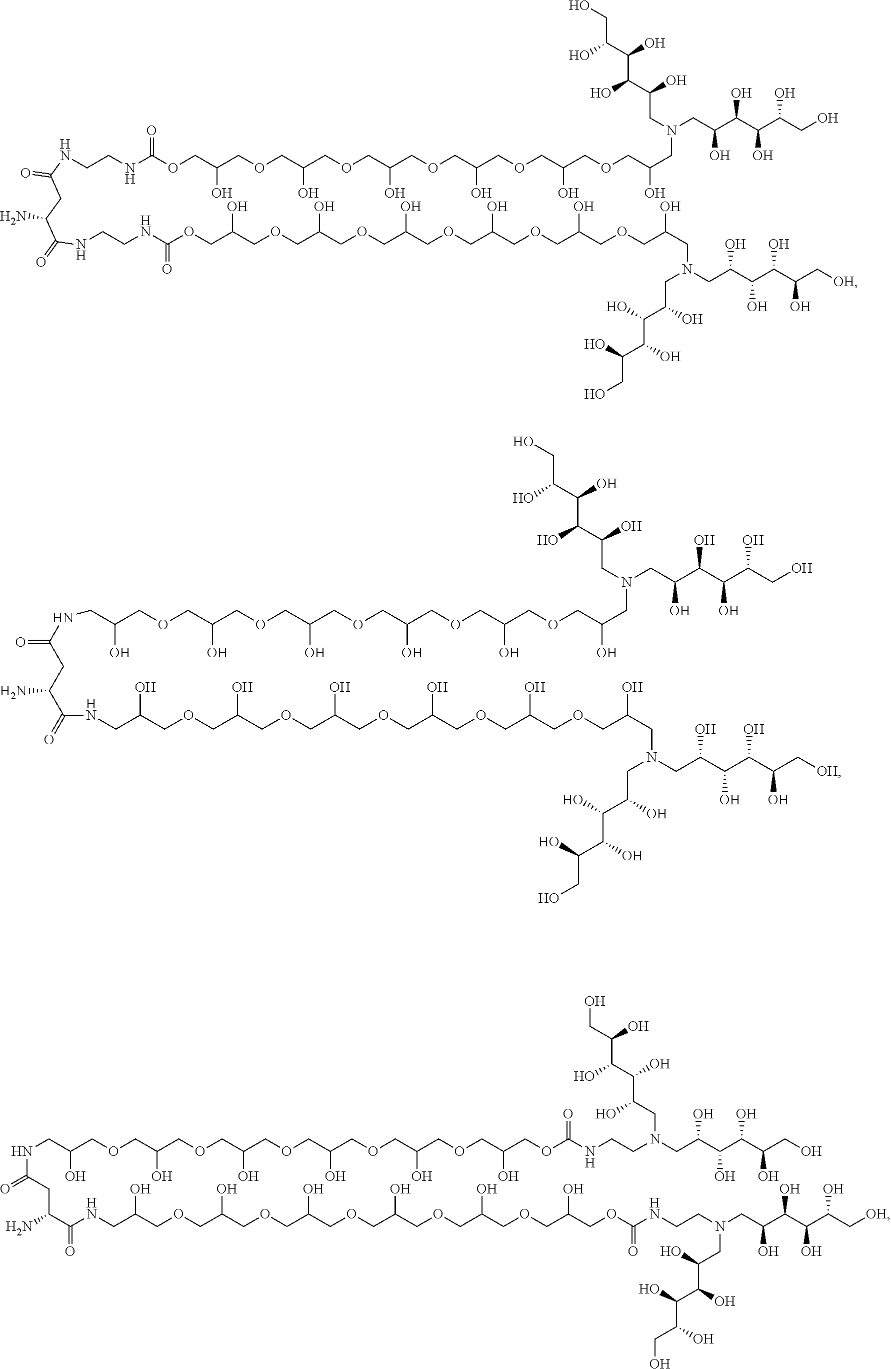

157. The Polar group of claim 140, wherein the Polar group is selected from

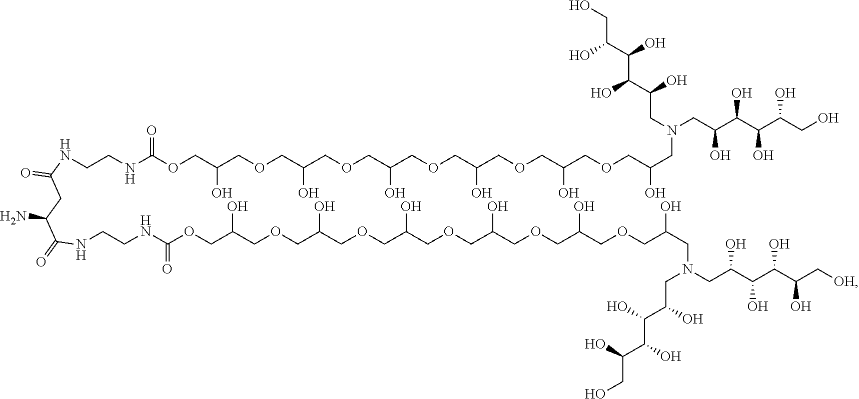 [SMILES: N[C@@H](CC(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

158. The Polar group of claim 1, represented by the formula:

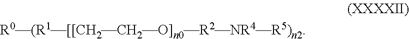

159. The Polar group of claim 158, wherein R⁰ is

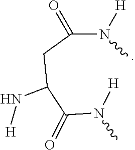

160. The Polar group of any one of claims 158 to 159, wherein R⁰ is a bond.

161. The Polar group of any one of claims 158 to 160, wherein n0 is 6 to 10.

162. The Polar group of any one of claims 158 to 161, wherein n0 is 8.

163. The Polar group of any one of claims 158 to 162, wherein R² is independently selected from a C₁-C₃ alkylene group.

164. The Polar group of any one of claims 158 to 163, wherein R⁴ and R⁵ are each a polyhydroxyl group.

165. The Polar group of any one of claims 158 to 164, wherein n2 is 2.

166. The Polar group of claim 140, wherein the Polar group is selected from

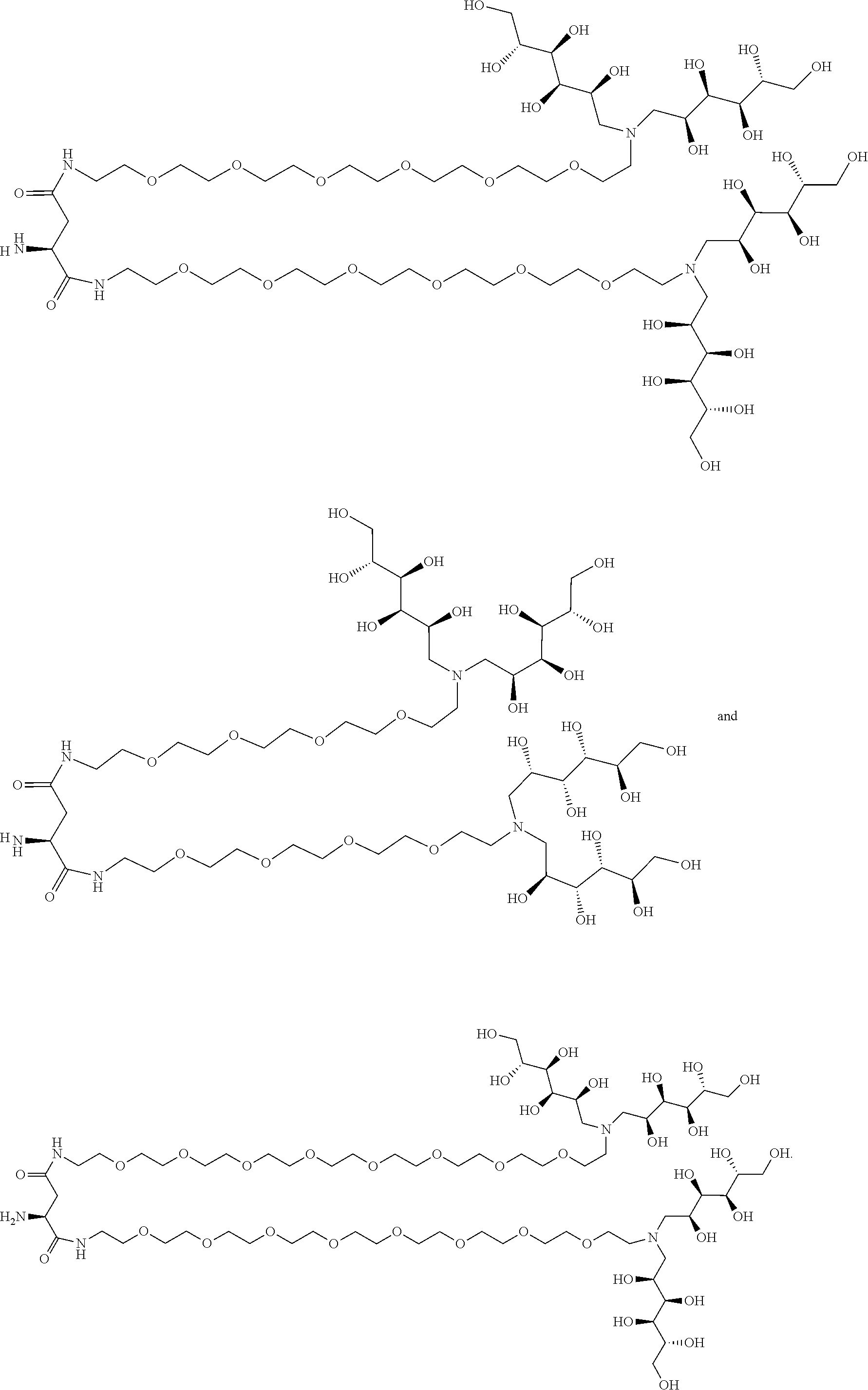

166. The Polar group of claim 140, wherein the Polar group is selected from 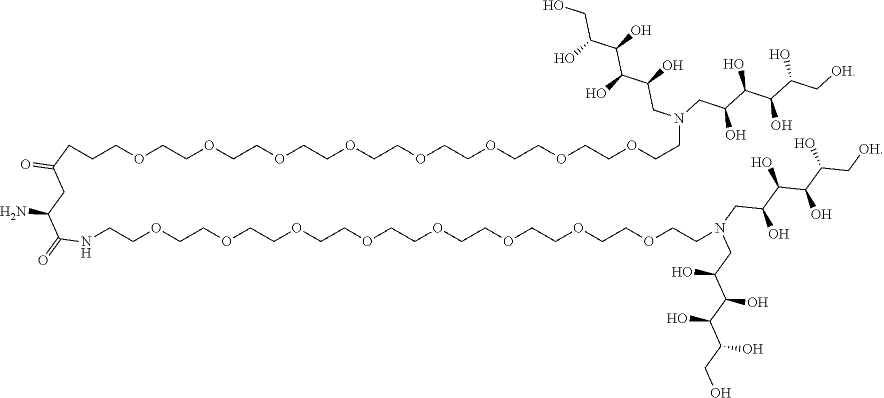 [SMILES: N[C@@H](CC(=O)CCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]
